(19) United States
(12) Patent Application Publication
(10) Pub. No.: US 2026/0327306 A1
(43) Pub. Date: Oct. 22, 2026

(54) ANTIBODY MOLECULES THAT BIND TO NKP30 AND USES THEREOF

(71) Applicant: Marengo Therapeutics, Inc., Cambridge, MA (US)

(72) Inventors: Madan Katragadda, Acton, MA (US); Andrew Bayliffe, London (GB)

(21) Appl. No.: 19/642,327

(22) Filed: Apr. 8, 2026

Related U.S. Application Data
(63) Continuation of application No. PCT/US24/50538, filed on Oct. 9, 2024.
(60) Provisional application No. 63/589,149, filed on Oct. 10, 2023.

(51) Int. Cl. C07K 16/28; C07K 14/54; C07K 14/55

(52) U.S. Cl. CPC C07K 16/2803; C07K 14/5443; C07K 14/55; C07K 16/2809; C07K 2317/24; C07K 2317/31; C07K 2317/565; C07K 2317/92; C07K 2319/00

(57) ABSTRACT

Provides herein are compositions comprising an agent that comprises an antigen binding domain that binds to NKp30, and methods of treating conditions or diseases in a subject and methods of expanding and/or activation NK cells using the same.

### CROSS REFERENCE

This application is a Continuation of U.S. International No. PCT/US2024/050538, filed on Oct. 9, 2024, which claims the benefit of U.S. provisional Application No. 63/589,149, filed on Oct. 10, 2023, the entire contents of which are incorporated herein by reference.

### SEQUENCE LISTING

The instant application contains a Sequence Listing which has been submitted electronically in XML format and is hereby incorporated by reference in its entirety. Said XML copy, created on Nov. 8, 2024, is named 53676-767_601_SL.xml and is 3,072,000 bytes in size.

### BACKGROUND

Natural Killer (NK) cells recognize and destroy tumors and virus-infected cells in an antibody-independent manner. The regulation of NK cells is mediated by activating and inhibiting receptors on the NK cell surface. One family of activating receptors is the natural cytotoxicity receptors (NCRs) which include NKp30, NKp44 and NKp46.

Given the importance of immune checkpoint pathways in regulating an immune response, the need exists for developing novel agents that modulate the activity of immunoinhibitory proteins, such as PD-1, thus leading to activation of immune system. Such agents can be used, e.g., for cancer immunotherapy and treatment of other conditions, such as chronic infection.

### SUMMARY

In an aspect, provided herein is, inter alia, a composition comprising an agent that comprises an antigen binding domain that binds to NKp30, wherein the antigen binding domain comprises a heavy chain variable region (VH) comprising:
(i) a heavy chain complementarity determining region 3 (VHCDR3) comprising the sequence of GDWHYFDY (SEQ ID NO: 504); and
(ii) a heavy chain complementarity determining region 1 (VHCDR1) comprising the sequence of GFSIIRVGYHWN (SEQ ID NO: 502), a heavy chain complementarity determining region 2 (VHCDR2) comprising the sequence of YIHRSGSTSYNPSLKS (SEQ ID NO: 503), or a combination thereof.

In some embodiments, the antigen binding domain comprises a VH comprising the VHCDR1 comprising the sequence of GFSIIRVGYHWN (SEQ ID NO: 502), the VHCDR2 comprising the sequence of YIHRSGSTSYNPSLKS (SEQ ID NO: 503), and the VHCDR3 comprising the sequence of GDWHYFDY (SEQ ID NO: 504).

In some embodiments, the antigen binding domain comprises a light chain variable region (VL) comprising a light chain complementarity determining region 1 (VLCDR1) comprising the sequence of SGEKLSDKYVH (SEQ ID NO: 26), a light chain complementarity determining region 2 (VLCDR2) comprising the sequence of ENDRRPS (SEQ ID NO: 28), a light chain complementarity determining region 3 (VLCDR3) comprising the sequences of QFWDSTNSAV (SEQ ID NO: 86), or any combination thereof.

In some embodiments, the antigen binding domain comprises a VL comprising a VLCDR1 comprising the sequence of SGEKLSDKYVH (SEQ ID NO: 26), a VLCDR2 comprising the sequence of ENDRRPS (SEQ ID NO: 28), and a VLCDR3 comprising the sequences of QFWDSTNSAV (SEQ ID NO: 86).

In some embodiments, the antigen binding domain comprises a VH comprising a sequence having at least 75% sequence identity to the sequence of SEQ ID NO: 505.

In some embodiments, the antigen binding domain comprises a VH comprising the sequence of SEQ ID NO: 505.

In some embodiments, the antigen binding domain comprises a VL comprising a sequence having at least 75% sequence identity to the sequence of SEQ ID NO: 13.

In some embodiments, the antigen binding domain comprises a VL comprising the sequence of SEQ ID NO: 13.

In some embodiments, the antigen binding domain comprises a VH comprising a sequence having at least 75% sequence identity to the sequence of SEQ ID NO: 505 and a VL comprising a sequence having at least 75% sequence identity to the sequence of SEQ ID NO: 13.

In some embodiments, the antigen binding domain comprises a VH comprising the sequence of SEQ ID NO: 505 and a VL comprising the sequence of SEQ ID NO: 13.

In some embodiments, the antigen binding domain is a full antibody or antigen-binding fragment thereof, a bivalent antibody, bispecific antibody, or a biparatopic antibody.

In some embodiments, the antigen binding domain is a Fab, F(ab′)2, Fv, an single chain Fv (scFv), a diabody, a single domain antibody, or a camelid antibody.

In some embodiments, the antigen binding domain is a Fab or an scFv.

In some embodiments, the antigen binding domain comprises one or more heavy chain constant regions selected from the group consisting of IgG1 heavy chain constant region or functional fragment thereof, IgG2 heavy chain constant region or functional fragment thereof, IgG3 heavy chain constant region or functional fragment thereof, IgGA1 heavy chain constant region or functional fragment thereof, IgGA2 heavy chain constant region or functional fragment thereof, IgG4 heavy chain constant region or functional fragment thereof, IgJ heavy chain constant region or functional fragment thereof, IgM heavy chain constant region or functional fragment thereof, IgD heavy chain constant region or functional fragment thereof, and IgE heavy chain constant region or functional fragment thereof.

In some embodiments, the one or more heavy chain constant regions comprises a sequence having at least 75% sequence identity to any one heavy chain constant region sequences listed in Tables 3, 7, 8, 16, 17, 39, 40, 42C, 43A, 43B, and 46-51.

In some embodiments, the one or more heavy chain constant regions comprises a sequence having at least 75% sequence identity to the sequence of SEQ ID NO: 73, the sequence of SEQ ID NO: 76, the sequence of SEQ ID NO: 74, the sequence of SEQ ID NO: 75, the sequence of SEQ ID NO: 76, the sequence of SEQ ID NO: 40, the sequence of SEQ ID NO: 41, the sequence of SEQ ID NO: 42, the sequence of SEQ ID NO: 3645, the sequence of SEQ ID NO: 3646, the sequence of SEQ ID NO: 3649, the sequence of SEQ ID NO: 3451, the sequence of SEQ ID NO: 506, the sequence of SEQ ID NO: 507, the sequence of SEQ ID NO: 3647, the sequence of SEQ ID NO: 3648, the sequence of SEQ ID NO: 508 or 8686, the sequence of SEQ ID NO: 513, the sequence of SEQ ID NO: 514, the sequence of SEQ ID NO: 515, the sequence of SEQ ID NO: 516, the sequence of SEQ ID NO: 3447, or the sequence of SEQ ID NO: 511.

In some embodiments, the antigen binding domain comprises a kappa light chain constant region or functional fragment thereof, a lambda light chain constant region or functional fragment thereof, or a combination thereof.

In some embodiments, the kappa light chain constant region comprises a sequence having at least 75% sequence identity to any one light chain constant region sequences listed in Tables 3, 7, 8, 16, 17, 39, 40, 42C, 43A, 43B, and 46-51.

In some embodiments, the kappa light chain constant region comprises a sequence having at least 75% sequence identity to the sequence of SEQ ID NO: 39 or the sequence of SEQ ID NO: 3644.

In some embodiments, the agent is a polypeptide molecule.

In some embodiments, the agent is a multifunctional molecule.

In some embodiments, the agent is a multispecific molecule.

In some embodiments, the agent comprises at least one cytokine molecule or a functional fragment or variant thereof or a functional fragment or variant thereof, an antigen binding domain that binds to T cell receptor beta chain constant domain (TRBC), a second antigen binding domain that binds to NKp30, an immune cell engager, a tumor-targeting moiety, or any combination thereof.

In some embodiments, the agent comprises at least two non-contiguous polypeptide chains comprising a first polypeptide chain and a second polypeptide chain;
wherein the first polypeptide chain comprises, from N-terminus to C-terminus, the following configuration:
A-[a first member of a dimerization module]-C,
And the second polypeptide chain comprises, from N-terminus to C-terminus, the following configuration:
B-[a second member of a dimerization module]-D;
wherein the first member of the dimerization module comprises a first immunoglobulin constant domain; and the second member of the dimerization module comprises a second immunoglobulin constant domain;
wherein at least one of A, B, C, and D is the antigen binding domain; and
wherein the first polypeptide chain and the second polypeptide chain dimerize via interaction between the first member of the dimerization module and the second member of the dimerization module.

In some embodiments, (i) A is the antigen binding domain, and B, C, and D are independently (a) absent, (b) at least one cytokine molecule or a functional fragment or variant thereof, (c) an antigen binding domain that binds to T cell receptor beta chain constant domain (TRBC), (d) a second antigen binding domain that binds to NKp30, (e) an immune cell engager, or (f) a tumor-targeting moiety;
(ii) B is the antigen binding domain, and A, C, and D are independently (a) absent, (b) at least one cytokine molecule or a functional fragment or variant thereof, (c) an antigen binding domain that binds to T cell receptor beta chain constant domain (TRBC), (d) a second antigen binding domain that binds to NKp30, (e) an immune cell engager, or (f) a tumor-targeting moiety;
(iii) C is the antigen binding domain, and A, B, and D are independently (a) absent, (b) at least one cytokine molecule or a functional fragment or variant thereof, (c) an antigen binding domain that binds to T cell receptor beta chain constant domain (TRBC), (d) a second antigen binding domain that binds to NKp30, (e) an immune cell engager, or (f) a tumor-targeting moiety;
(iv) D is the antigen binding domain, and A, B, and C are independently (a) absent, (b) at least one cytokine molecule or a functional fragment or variant thereof, (c) an antigen binding domain that binds to T cell receptor beta chain constant domain (TRBC), (d) a second antigen binding domain that binds to NKp30, (e) an immune cell engager, or (f) a tumor-targeting moiety.

In some embodiments, the agent comprises at least one cytokine molecule or a functional fragment or variant thereof.

In some embodiments, the at least one cytokine molecule or a functional fragment or variant thereof comprises IL-2 or a functional fragment or variant thereof, IL-7 or a functional fragment or variant thereof, IL-12 or a functional fragment or variant thereof, IL-15 or a functional fragment or variant thereof, IL-18 or a functional fragment or variant thereof, IL-21 or a functional fragment or variant thereof, interferon gamma or a functional fragment or variant thereof, or any combination thereof.

In some embodiments, the at least one cytokine molecule or a functional fragment or variant thereof comprises a sequence having at least 75% sequence identity to any one sequence selected from the group consisting of the cytokine sequences listed in Tables 40, 42C, 43A, 43B, and 46-51.

In some embodiments, the at least one cytokine molecule or a functional fragment or variant thereof comprises any one sequence selected from the group consisting of the cytokine sequences listed in Tables 40, 42C, 43A, 43B, and 46-51.

In some embodiments, the at least one cytokine molecule or a functional fragment or variant thereof comprises IL-2 or a functional fragment or variant thereof.

In some embodiments, the IL-2 or a functional fragment or variant thereof comprises a sequence having at least 75% sequence identity to the sequence of SEQ ID NO: 2191, the sequence of SEQ ID NO: 2270, or the sequence of SEQ ID NO: 2280.

In some embodiments, the IL-2 or a functional fragment or variant thereof comprises a sequence the sequence of SEQ ID NO: 2191, the sequence of SEQ ID NO: 2270, or the sequence of SEQ ID NO: 2280.

In some embodiments, the at least one cytokine molecule or a functional fragment or variant thereof IL-15 or a functional fragment or variant thereof.

In some embodiments, the IL-15 or a functional fragment or variant thereof comprises a sequence having at least 75% sequence identity to the sequence of SEQ ID NO: 2170, SEQ ID NO: 3670, or SEQ ID NO: 3673.

In some embodiments, the IL-15 or a functional fragment or variant thereof comprises the sequence of SEQ ID NO: 2170, the sequence of SEQ ID NO: 3670, or the sequence of SEQ ID NO: 3673.

In some embodiments, the at least one cytokine molecule or a functional fragment or variant thereof further comprises an IL15Ralpha dimerizing domain covalently linked the IL-15 or a functional fragment or variant thereof.

In some embodiments, the IL15Ralpha dimerizing domain comprises an IL-15 receptor alpha sushi domain or a functional fragment or variant thereof.

In some embodiments, the IL15Ralpha dimerizing domain comprises a sequence having at least 75% sequence identity to the sequence of SEQ ID NO: 3472.

In some embodiments, the IL15Ralpha dimerizing domain comprises the sequence of SEQ ID NO: 3472.

In some embodiments, the IL-15 or a functional fragment or variant thereof is covalently linked to the IL15Ralpha dimerizing domain via a linker.

In some embodiments, the IL-15 or a functional fragment or variant thereof is covalently linked to the IL15Ralpha dimerizing domain via a linker comprising the sequence of SEQ ID NO: 3473.

In some embodiments, the at least one cytokine molecule or a functional fragment or variant thereof comprises a sequence having at least 75% sequence identity to the sequence of SEQ ID NO: 3474, SEQ ID NO: 3671, or SEQ ID NO: 8689.

In some embodiments, the at least one cytokine molecule or a functional fragment or variant thereof comprises the sequence of SEQ ID NO: 3474, the sequence of SEQ ID NO: 3671, or the sequence of SEQ ID NO: 8689.

In some embodiments, the at least one cytokine molecule or a functional fragment or variant thereof comprises IL-7 or a functional fragment or variant thereof.

In some embodiments, the IL-7 or the functional fragment or variant thereof comprises a sequence having at least 75% sequence identity to the sequence of SEQ ID NO: 3540.

In some embodiments, the IL-7 or the functional fragment or variant thereof comprises the sequence of SEQ ID NO: 3540.

In some embodiments, the at least one cytokine molecule or a functional fragment or variant thereof comprises IL-12 or a functional fragment or variant or variant thereof.

In some embodiments, the IL-12 or the functional fragment or variant thereof comprises a sequence having at least 75% sequence identity to the sequence of SEQ ID NO: 3542, the sequence of SEQ ID NO: 3543, or the sequence of SEQ ID NO: 3545.

In some embodiments, the IL-12 or the functional fragment or variant thereof comprises the sequence of SEQ ID NO: 3542, the sequence of SEQ ID NO: 3543, or the sequence of SEQ ID NO: 3545.

In some embodiments, the at least one cytokine molecule or a functional fragment or variant thereof comprises IL-18 of a functional fragment or variant thereof.

In some embodiments, the IL-18 or the functional fragment or variant thereof comprises a sequence having at least 75% sequence identity to the sequence of SEQ ID NO: 2192.

In some embodiments, the IL-18 or the functional fragment or variant thereof comprises the sequence of SEQ ID NO: 2192.

In some embodiments, the at least one cytokine molecule or a functional fragment or variant thereof comprises IL-21 or a functional fragment or variant thereof.

In some embodiments, the IL-21 or the functional fragment or variant thereof comprises a sequence having at least 75% sequence identity to the sequence of SEQ ID NO: 3540.

In some embodiments, the IL-21 or the functional fragment or variant thereof comprises the sequence of SEQ ID NO: 3540.

In some embodiments, the agent comprises an antigen binding domain that binds to TRBC.

In some embodiments, the antigen binding domain that binds to TRBC binds to TRBC1 or TRBC2.

In some embodiments, the antigen binding domain that binds to TRBC binds to TRBC1, and wherein the antigen binding domain that binds to TRBC comprises a VH comprising:
(i) a VHCDR1 comprising a sequence having at least 75% sequence identity to any one sequence selected from the group consisting of the VHCDR1 sequences listed in Tables 2A, 2B, 3A, 3B, 5A, 5B, 7, 8, 16, 39, and 40;
(ii) a VHCDR2 comprising a sequence having at least 75% sequence identity any one sequence selected from the group consisting of the VHCDR2 sequences listed in Tables 2A, 2B, 3A, 3B, 5A, 5B, 7, 8, 16, 39, and 40;
(iii) a VHCDR3 comprising a sequence having at least 75% sequence identity to any one sequence selected from the group consisting of the VHCDR3 sequences listed in Tables 2A, 2B, 3A, 3B, 5A, 5B, 7, 8, 16, 39, and 40; or
(iv) any combination thereof.

In some embodiments, the antigen binding domain that binds to TRBC binds to TRBC1, and wherein the antigen binding domain that binds to TRBC comprises a VH comprising:
(i) a VHCDR1 comprising to any one sequence selected from the group consisting of the VHCDR1 sequences listed in Tables 2A, 2B, 3A, 3B, 5A, 5B, 7, 8, 16, 39, and 40;
(ii) a VHCDR2 comprising any one sequence selected from the group consisting of the VHCDR2 sequences listed in Tables 2A, 2B, 3A, 3B, 5A, 5B, 7, 8, 16, 39, and 40;
(iii) a VHCDR3 comprising any one sequence selected from the group consisting of the VHCDR3 sequences listed in Tables 2A, 2B, 3A, 3B, 5A, 5B, 7, 8, 16, 39, and 40; or
(iv) any combination thereof.

In some embodiments, the antigen binding domain that binds to TRBC binds to TRBC1, and wherein the antigen binding domain that binds to TRBC comprises a VH comprising a sequence having at least 75% sequence identity to any one sequence selected from the group consisting of the VH sequences listed in Tables 7, 8, 16, 39, and 40.

In some embodiments, the antigen binding domain that binds to TRBC binds to TRBC1, and wherein the antigen binding domain that binds to TRBC comprises a VH comprising any one sequence selected from the group consisting of the VH sequences listed in Tables 7, 8, 16, 39, and 40.

In some embodiments, the antigen binding domain that binds to TRBC binds to TRBC1, and wherein the antigen binding domain that binds to TRBC comprises a VL comprising:
(i) a VLCDR1 comprising a sequence having at least 75% sequence identity to any one sequence selected from the group consisting of the VLCDR1 sequences listed in Tables 4, 6, 7, 8, 16, 39, and 40;
(ii) a VLCDR2 comprising a sequence having at least 75% sequence identity any one sequence selected from the group consisting of the VLCDR2 sequences listed in Tables 4, 6, 7, 8, 16, 39, and 40;
(iii) a VLCDR3 comprising a sequence having at least 75% sequence identity to any one sequence selected from the group consisting of the VLCDR3 sequences listed in Tables 4, 6, 7, 8, 16, 39, and 40; or
(iv) any combination thereof.

In some embodiments, the antigen binding domain that binds to TRBC binds to TRBC1, and wherein the antigen binding domain that binds to TRBC comprises a VL comprising:
(i) a VLCDR1 comprising any one sequence selected from the group consisting of the VLCDR1 sequences listed in Tables 4, 6, 7, 8, 16, 39, and 40;
(ii) a VLCDR2 comprising any one sequence selected from the group consisting of the VLCDR2 sequences listed in Tables 4, 6, 7, 8, 16, 39, and 40;
(iii) a VLCDR3 comprising any one sequence selected from the group consisting of the VLCDR3 sequences listed in Tables 4, 6, 7, 8, 16, 39, and 40; or
(iv) any combination thereof.

In some embodiments, the antigen binding domain that binds to TRBC binds to TRBC1, and wherein the antigen binding domain that binds to TRBC comprises a VL comprising a sequence having at least 75% sequence identity to any one sequence selected from the group consisting of the VL sequences listed in Tables 7, 8, 16, 39, and 40.

In some embodiments, the antigen binding domain that binds to TRBC binds to TRBC1, and wherein the antigen binding domain that binds to TRBC comprises a VL comprising any one sequence selected from the group consisting of the VL sequences listed in Tables 7, 8, 16, 39, and 40.

In some embodiments, the antigen binding domain that binds to TRBC binds to TRBC2, and wherein the antigen binding domain that binds to TRBC comprises a VH comprising:
(i) a VHCDR1 comprising a sequence having at least 75% sequence identity to any one sequence selected from the group consisting of the VHCDR1 sequences listed in Tables 9A, 9B, 11, 12, 13, 14, 15, 17, 39, and 40;
(ii) a VHCDR2 comprising a sequence having at least 75% sequence identity any one sequence selected from the group consisting of the VHCDR2 sequences listed in Tables 9A, 9B, 12, 13, 14, 15, 17, 39, and 40;
(iii) a VHCDR3 comprising a sequence having at least 75% sequence identity to any one sequence selected from the group consisting of the VHCDR3 sequences listed in Tables 9A, 9B, 12, 13, 14, 15, 17, 39, and 40; or
(iv) any combination thereof.

In some embodiments, the antigen binding domain that binds to TRBC binds to TRBC2, and wherein the antigen binding domain that binds to TRBC comprises a VH comprising:
(i) a VHCDR1 comprising to any one sequence selected from the group consisting of the VHCDR1 sequences listed in Tables 9A, 9B, 12, 13, 14, 15, 17, 39, and 40;
(ii) a VHCDR2 comprising any one sequence selected from the group consisting of the VHCDR2 sequences listed in Tables 9A, 9B, 12, 13, 14, 15, 17, 39, and 40;
(iii) a VHCDR3 comprising any one sequence selected from the group consisting of the VHCDR3 sequences listed in Tables 9A, 9B, 12, 13, 14, 15, 17, 39, and 40; or
(iv) any combination thereof.

In some embodiments, the antigen binding domain that binds to TRBC binds to TRBC2, and wherein the antigen binding domain that binds to TRBC comprises a VH comprising a sequence having at least 75% sequence identity to any one sequence selected from the group consisting of the VH sequences listed in Tables 11, 15, 17, 39, and 40.

In some embodiments, the antigen binding domain that binds to TRBC binds to TRBC2, and wherein the antigen binding domain that binds to TRBC comprises a VH comprising any one sequence selected from the group consisting of the VH sequences listed in Tables 11, 15, 17, 39, and 40.

In some embodiments, the antigen binding domain that binds to TRBC binds to TRBC2, and wherein the antigen binding domain that binds to TRBC comprises a VL comprising:
(i) a VLCDR1 comprising a sequence having at least 75% sequence identity to any one sequence selected from the group consisting of the VLCDR1 sequences listed in Tables 10, 12, 13, 14, 15, 17, 39, and 40;
(ii) a VLCDR2 comprising a sequence having at least 75% sequence identity any one sequence selected from the group consisting of the VLCDR2 sequences listed in Tables 10, 12, 13, 14, 15, 17, 39, and 40;
(iii) a VLCDR3 comprising a sequence having at least 75% sequence identity to any one sequence selected from the group consisting of the VLCDR3 sequences listed in Tables 10, 12, 13, 14, 15, 17, 39, and 40; or
(iv) any combination thereof.

In some embodiments, the antigen binding domain that binds to TRBC binds to TRBC2, and wherein the antigen binding domain that binds to TRBC comprises a VL comprising:
(i) a VLCDR1 comprising any one sequence selected from the group consisting of the VLCDR1 sequences listed in Tables 10, 12, 13, 14, 15, 17, 39, and 40;
(ii) a VLCDR2 comprising any one sequence selected from the group consisting of the VLCDR2 sequences listed in Tables 10, 12, 13, 14, 15, 17, 39, and 40;
(iii) a VLCDR3 comprising any one sequence selected from the group consisting of the VLCDR3 sequences listed in Tables 10, 12, 13, 14, 15, 17, 39, and 40; or
(iv) any combination thereof.

In some embodiments, the antigen binding domain that binds to TRBC binds to TRBC2, and wherein the antigen binding domain that binds to TRBC comprises a VL comprising a sequence having at least 75% sequence identity to any one sequence selected from the group consisting of the VL sequences listed in Tables 11, 15, 17, 39, and 40.

In some embodiments, the antigen binding domain that binds to TRBC binds to TRBC2, and wherein the antigen binding domain that binds to TRBC comprises a VL comprising any one sequence selected from the group consisting of the VL sequences listed in Tables 11, 15, 17, 39, and 40.

In some embodiments, the antigen binding domain that binds to TRBC binds to TRBC2, and wherein the antigen binding domain that binds to TRBC comprises a sequence having at least 75% sequence identity to any one sequence selected from the group consisting of the scFv sequences listed in Tables 12, 15, 17, 39, and 40.

In some embodiments, the antigen binding domain that binds to TRBC binds to TRBC2, and wherein the antigen binding domain that binds to TRBC comprises a VL comprising any one sequence selected from the group consisting of the scFv sequences listed in Tables 12, 15, 17, 39, and 40.

In some embodiments, the agent comprises a second antigen binding domain that binds to NKp30.

In some embodiments, the antigen binding domain and the second antigen binding domain that binds to NKp30 are same.

In some embodiments, the antigen binding domain and the second antigen binding domain that binds to NKp30 are different.

In some embodiments, the second antigen binding domain that binds to NKp30 comprises a heavy chain variable region (VH) comprising:
(i) a heavy chain complementarity determining region 3 (VHCDR3) comprising the sequence of GDWHYFDY (SEQ ID NO: 504); and
(ii) a heavy chain complementarity determining region 1 (VHCDR1) comprising the sequence of GFSIIRVGYHWN (SEQ ID NO: 502), a heavy chain complementarity determining region 2 (VHCDR2) comprising the sequence of YIHRSGSTSYNPSLKS (SEQ ID NO: 503), or a combination thereof.

In some embodiments, the second antigen binding domain that binds to NKp30 comprises a VH comprising the VHCDR1 comprising the sequence of GFSIIRVGYHWN (SEQ ID NO: 502), the VHCDR2 comprising the sequence of YIHRSGSTSYNPSLKS (SEQ ID NO: 503), and the VHCDR3 comprising the sequence of GDWHYFDY (SEQ ID NO: 504).

In some embodiments, the second antigen binding domain that binds to NKp30 comprises a light chain variable region (VL) comprising a light chain complementarity determining region 1 (VLCDR1) comprising the sequence of SGEKLSDKYVH (SEQ ID NO: 26), a light chain complementarity determining region 2 (VLCDR2) comprising the sequence of ENDRRPS (SEQ ID NO: 28), a light chain complementarity determining region 3 (VLCDR3) comprising the sequences of QFWDSTNSAV (SEQ ID NO: 86), or any combination thereof.

In some embodiments, the second antigen binding domain that binds to NKp30 comprises a VL comprising a VLCDR1 comprising the sequence of SGEKLSDKYVH (SEQ ID NO: 26), a VLCDR2 comprising the sequence of ENDRRPS (SEQ ID NO: 28), and a VLCDR3 comprising the sequences of QFWDSTNSAV (SEQ ID NO: 86).

In some embodiments, the second antigen binding domain that binds to NKp30 comprises a VH comprising a sequence having at least 75% sequence identity to the sequence of SEQ ID NO: 505.

In some embodiments, the second antigen binding domain that binds to NKp30 comprises a VH comprising the sequence of SEQ ID NO: 505.

In some embodiments, the second antigen binding domain that binds to NKp30 comprises a VL comprising a sequence having at least 75% sequence identity to the sequence of SEQ ID NO: 13.

In some embodiments, the second antigen binding domain that binds to NKp30 comprises a VL comprising the sequence of SEQ ID NO: 13.

In some embodiments, the second antigen binding domain that binds to NKp30 comprises a VH comprising a sequence having at least 75% sequence identity to the sequence of SEQ ID NO: 505 and a VL comprising a sequence having at least 75% sequence identity to the sequence of SEQ ID NO: 13.

In some embodiments, the second antigen binding domain that binds to NKp30 comprises a VH comprising the sequence of SEQ ID NO: 505 and a VL comprising the sequence of SEQ ID NO: 13.

In some embodiments, the second antigen binding domain that binds to NKp30 comprises a heavy chain variable region (VH) comprising:
(i) a VHCDR1 comprising a sequence having at least 75% sequence identity to any one sequence selected from the group consisting of the VHCDR1 sequences listed in Tables 20A, 20B, 21A, 21B, 23A, 23B, 25, 26, 16, and 17;
(ii) a VHCDR2 comprising a sequence having at least 75% sequence identity any one sequence selected from the group consisting of the VHCDR2 sequences listed in Tables 20A, 20B, 21A, 21B, 23A, 23B, 25, 26, 16, and 17;
(iii) a VHCDR3 comprising a sequence having at least 75% sequence identity to any one sequence selected from the group consisting of the VHCDR3 sequences listed in Tables 20A, 20B, 21A, 21B, 23A, 23B, 25, 26, 16, and 17; or
(iv) any combination thereof.

In some embodiments, the second antigen binding domain that binds to NKp30 comprises a VH comprising:
(i) a VHCDR1 comprising to any one sequence selected from the group consisting of the VHCDR1 sequences listed in Tables 20A, 20B, 21A, 21B, 23A, 23B, 25, 26, 16, and 17;
(ii) a VHCDR2 comprising any one sequence selected from the group consisting of the VHCDR2 sequences listed in Tables 20A, 20B, 21A, 21B, 23A, 23B, 25, 26, 16, and 17;
(iii) a VHCDR3 comprising any one sequence selected from the group consisting of the VHCDR3 sequences listed in Tables 20A, 20B, 21A, 21B, 23A, 23B, 25, 26, 16, and 17; or
(iv) any combination thereof.

In some embodiments, the second antigen binding domain that binds to NKp30 comprises a VH comprising a sequence having at least 75% sequence identity to any one sequence selected from the group consisting of the VH sequences listed in Tables 25, 26, 16, and 17.

In some embodiments, the second antigen binding domain that binds to NKp30 comprises a VH comprising any one sequence selected from the group consisting of the VH sequences listed in Tables 25, 26, 16, and 17.

In some embodiments, the second antigen binding domain that binds to NKp30 comprises a VL comprising:
(i) a VLCDR1 comprising a sequence having at least 75% sequence identity to any one sequence selected from the group consisting of the VLCDR1 sequences listed in Tables 22, 24, 25, 26, 16, and 17;
(ii) a VLCDR2 comprising a sequence having at least 75% sequence identity any one sequence selected from the group consisting of the VLCDR2 sequences listed in Tables 22, 24, 25, 26, 16, and 17;
(iii) a VLCDR3 comprising a sequence having at least 75% sequence identity to any one sequence selected from the group consisting of the VLCDR3 sequences listed in Tables 22, 24, 25, 26, 16, and 17; or
(iv) any combination thereof.

In some embodiments, the second antigen binding domain that binds to NKp30 comprises a VL comprising:
(i) a VLCDR1 comprising any one sequence selected from the group consisting of the VLCDR1 sequences listed in Tables 22, 24, 25, 26, 16, and 17;
(ii) a VLCDR2 comprising any one sequence selected from the group consisting of the VLCDR2 sequences listed in Tables 22, 24, 25, 26, 16, and 17;
(iii) a VLCDR3 comprising any one sequence selected from the group consisting of the VLCDR3 sequences listed in Tables 22, 24, 25, 26, 16, and 17; or
(iv) any combination thereof.

In some embodiments, the second antigen binding domain that binds to NKp30 comprises a VL comprising a sequence having at least 75% sequence identity to any one sequence selected from the group consisting of the VL sequences listed in Tables 25, 26, 16, and 17.

In some embodiments, the second antigen binding domain that binds to NKp30 comprises a VL comprising any one sequence selected from the group consisting of the VL sequences listed in Tables 25, 26, 16, and 17.

In some embodiments, the agent comprises an immune cell engager.

In some embodiments, the immune cell engager is selected from the group consisting of a T cell engager, an NK cell engager, a B cell engager, a dendritic cell engager, and the macrophage cell engager.

In some embodiments, the agent comprises a T cell engager.

In some embodiments, the T cell engager comprises an antigen binding domain that binds to a T cell receptor beta chain region (TCRβV).

In some embodiments, the antigen binding domain that binds to a T cell receptor beta chain region (TCRβV) binds to a TCRβV of any one T cell receptor beta chain (TCRβ) subfamily member selected from the group consisting of TCRβ subfamily members listed in Tables 29 and 42A.

In some embodiments, the antigen binding domain that binds to a TCRβV comprises a heavy chain variable region (VH) comprising:
(i) a VHCDR1 comprising a sequence having at least 75% sequence identity to any one sequence selected from the group consisting of the VHCDR1 sequences listed in Tables 30, 32, 42B, 42C, 43A, 43B, and 43-51;
(ii) a VHCDR2 comprising a sequence having at least 75% sequence identity any one sequence selected from the group consisting of the VHCDR2 sequences listed in Tables 30, 32, 42B, 42C, 43A, 43B, and 43-51;
(iii) a VHCDR3 comprising a sequence having at least 75% sequence identity to any one sequence selected from the group consisting of the VHCDR3 sequences listed in Tables 30, 32, 42B, 42C, 43A, 43B, and 43-51; or
(iv) any combination thereof.

In some embodiments, the antigen binding domain that binds to a TCRβV comprises a VH comprising:
(i) a VHCDR1 comprising to any one sequence selected from the group consisting of the VHCDR1 sequences listed in Tables 30, 32, 42B, 42C, 43A, 43B, and 43-51;
(ii) a VHCDR2 comprising any one sequence selected from the group consisting of the VHCDR2 sequences listed in Tables 30, 32, 42B, 42C, 43A, 43B, and 43-51;
(iii) a VHCDR3 comprising any one sequence selected from the group consisting of the VHCDR3 sequences listed in Tables 30, 32, 42B, 42C, 43A, 43B, and 43-51; or
(iv) any combination thereof.

In some embodiments, the antigen binding domain that binds to a TCRβV comprises a VH comprising a sequence having at least 75% sequence identity to any one sequence selected from the group consisting of the VH sequences listed in Tables 30, 32, 42B, 42C, 43A, 43B, and 43-51.

In some embodiments, the antigen binding domain that binds to a TCRβV comprises a VH comprising any one sequence selected from the group consisting of the VH sequences listed in Tables 30, 32, 42B, 42C, 43A, 43B, and 43-51.

In some embodiments, the antigen binding domain that binds to a TCRβV comprises a VL comprising:
(i) a VLCDR1 comprising a sequence having at least 75% sequence identity to any one sequence selected from the group consisting of the VLCDR1 sequences listed in Tables 30, 32, 42B, 42C, 43A, 43B, and 43-51;
(ii) a VLCDR2 comprising a sequence having at least 75% sequence identity any one sequence selected from the group consisting of the VLCDR2 sequences listed in Tables 30, 32, 42B, 42C, 43A, 43B, and 43-51;
(iii) a VLCDR3 comprising a sequence having at least 75% sequence identity to any one sequence selected from the group consisting of the VLCDR3 sequences listed in Tables 30, 32, 42B, 42C, 43A, 43B, and 43-51; or
(iv) any combination thereof.

In some embodiments, the antigen binding domain that binds to a TCRβV comprises a VL comprising:
(i) a VLCDR1 comprising any one sequence selected from the group consisting of the VLCDR1 sequences listed in Tables 30, 32, 42B, 42C, 43A, 43B, and 43-51;
(ii) a VLCDR2 comprising any one sequence selected from the group consisting of the VLCDR2 sequences listed in Tables 30, 32, 42B, 42C, 43A, 43B, and 43-51;
(iii) a VLCDR3 comprising any one sequence selected from the group consisting of the VLCDR3 sequences listed in Tables 30, 32, 42B, 42C, 43A, 43B, and 43-51; or
(iv) any combination thereof.

In some embodiments, the antigen binding domain that binds to a TCRβV comprises a VL comprising a sequence having at least 75% sequence identity to any one sequence selected from the group consisting of the VL sequences listed in Tables 30, 32, 42B, 42C, 43A, 43B, and 43-51.

In some embodiments, the antigen binding domain that binds to a TCRβV comprises a VL comprising any one sequence selected from the group consisting of the VL sequences listed in Tables 30, 32, 42B, 42C, 43A, 43B, and 43-51.

In some embodiments, the agent comprises a NK cell engager.

In some embodiments, the NK cell engager does not bind to NKp30.

In some embodiments, the NK cell engager comprise a sequence having at least 75% sequence identity to any one sequence selected from the group consisting of the sequences listed in Table 27.

In some embodiments, the NK cell engager comprise any one sequence selected from the group consisting of the sequences listed in Table 27.

In some embodiments, the agent comprises a tumor-targeting moiety.

In some embodiments, the antigen binding domain that binds TRBC, the second antigen binding domain that binds to NKp30, the immune cell engager, the tumor-targeting moiety, or any combination thereof is a full antibody or antigen-binding fragment thereof, a bivalent antibody, bispecific antibody, or a biparatopic antibody.

In some embodiments, the antigen binding domain that binds TRBC, the second antigen binding domain that binds to NKp30, the immune cell engager, the tumor-targeting moiety, or any combination thereof is a Fab, F(ab′)2, Fv, an single chain Fv (scFv), a diabody, a single domain antibody, or a camelid antibody.

In some embodiments, the antigen binding domain that binds TRBC, the second antigen binding domain that binds to NKp30, the immune cell engager, the tumor-targeting moiety, or any combination thereof is a Fab or an scFv.

In some embodiments, the antigen binding domain that binds TRBC, the second antigen binding domain that binds to NKp30, the immune cell engager, the tumor-targeting moiety, or any combination thereof comprises one or more heavy chain constant regions selected from the group consisting of IgG1 heavy chain constant region or functional fragment thereof, IgG2 heavy chain constant region or functional fragment thereof, IgG3 heavy chain constant region or functional fragment thereof, IgGA1 heavy chain constant region or functional fragment thereof, IgGA2 heavy chain constant region or functional fragment thereof, IgG4 heavy chain constant region or functional fragment thereof, IgJ heavy chain constant region or functional fragment thereof, IgM heavy chain constant region or functional fragment thereof, IgD heavy chain constant region or functional fragment thereof, and IgE heavy chain constant region or functional fragment thereof.

In some embodiments, the one or more heavy chain constant regions comprises a sequence having at least 75% sequence identity to any one heavy chain constant region sequences listed in Tables 3, 7, 8, 16, 17, 39, 40, 42C, 43A, 43B, and 46-51.

In some embodiments, the one or more heavy chain constant regions comprises a sequence having at least 75% sequence identity to the sequence of SEQ ID NO: 73, the sequence of SEQ ID NO: 76, the sequence of SEQ ID NO: 74, the sequence of SEQ ID NO: 75, the sequence of SEQ ID NO: 76, the sequence of SEQ ID NO: 40, the sequence of SEQ ID NO: 41, the sequence of SEQ ID NO: 42, the sequence of SEQ ID NO: 3645, the sequence of SEQ ID NO: 3646, the sequence of SEQ ID NO: 3649, the sequence of SEQ ID NO: 3451, the sequence of SEQ ID NO: 506, the sequence of SEQ ID NO: 507, the sequence of SEQ ID NO: 3647, the sequence of SEQ ID NO: 3648, the sequence of SEQ ID NO: 508 or 8686, the sequence of SEQ ID NO: 513, the sequence of SEQ ID NO: 514, the sequence of SEQ ID NO: 515, the sequence of SEQ ID NO: 516, the sequence of SEQ ID NO: 3447, or the sequence of SEQ ID NO: 511.

In some embodiments, the antigen binding domain that binds TRBC, the second antigen binding domain that binds to NKp30, the immune cell engager, the tumor-targeting moiety, or any combination thereof comprises a kappa light chain constant region or functional fragment thereof, a lambda light chain constant region or functional fragment thereof, or a combination thereof.

In some embodiments, the kappa light chain constant region comprises a sequence having at least 75% sequence identity to any one light chain constant region sequences listed in Tables 3, 7, 8, 16, 17, 39, 40, 42C, 43A, 43B, and 46-51.

In some embodiments, the kappa light chain constant region comprises a sequence having at least 75% sequence identity to the sequence of SEQ ID NO: 39 or the sequence of SEQ ID NO: 3644.

In some embodiments, the first member of the dimerization module comprises a first Fc region and the second member of the dimerization module comprises a second Fc region.

In some embodiments, the first Fc region is selected from the group consisting of an IgG1 Fc region or a functional fragment thereof, an IgG2 Fc region or a functional fragment thereof, an IgG3 Fc region or a functional fragment thereof, an IgGA1 Fc region or a functional fragment thereof, an IgGA2 Fc region or a functional fragment thereof, an IgG4 Fc region or a functional fragment thereof, an IgJ Fc region or a functional fragment thereof, an IgM Fc region or a functional fragment thereof, an IgD Fc region or a functional fragment thereof, and an IgE Fc region or a functional fragment thereof.

In some embodiments, the second Fc region is selected from the group consisting of an IgG1 Fc region or a functional fragment thereof, an IgG2 Fc region or a functional fragment thereof, an IgG3 Fc region or a functional fragment thereof, an IgGA1 Fc region or a functional fragment thereof, an IgGA2 Fc region or a functional fragment thereof, an IgG4 Fc region or a functional fragment thereof, an IgJ Fc region or a functional fragment thereof, an IgM Fc region or a functional fragment thereof, an IgD Fc region or a functional fragment thereof, and an IgE Fc region or a functional fragment thereof.

In some embodiments, the first Fc region, the second Fc region, or a combination thereof comprises an Fe interface with one or more of: a paired cavity-protuberance, an electrostatic interaction, or a strand-exchange, wherein the dimerization of the first Fc region and the second Fc region is enhanced as indicated by a greater ratio of heteromultimer:homomultimer forms relative to a dimerization of Fc regions with a non-engineered interface.

In some embodiments,
(i) the first Fc region comprises:
(a) an Ala at position 297 in a heavy chain constant region according to EU Numbering
(b) a Cys at position 349 in a heavy chain constant region according to EU Numbering),
(c) a Ser at position 366 in a heavy chain constant region according to EU Numbering),
(d) an Ala at position 368 in a heavy chain constant region according to EU Numbering); and
(e) a Val at position 407 in a heavy chain constant region according to EU Numbering); and
(ii) the second Fc region comprises:
(a) an Ala at position 297 in a heavy chain constant region according to EU Numbering),
(b) a Cys at position 354 in a heavy chain constant region according to EU Numbering), and
(c) a Trp at position 366 in a heavy chain constant region according to EU Numbering).

In some embodiments,
(i) the first Fc region comprises:
(a) an Ala at position 297 in a heavy chain constant region according to EU Numbering),
(b) a Cys at position 354 in a heavy chain constant region according to EU Numbering), and
(c) a Trp at position 366 in a heavy chain constant region according to EU Numbering); and
(ii) the second Fc region comprises:
(a) an Ala at position 297 in a heavy chain constant region according to EU Numbering
(b) a Cys at position 349 in a heavy chain constant region according to EU Numbering),
(c) a Ser at position 366 in a heavy chain constant region according to EU Numbering),
(d) an Ala at position 368 in a heavy chain constant region according to EU Numbering); and
(e) a Val at position 407 in a heavy chain constant region according to EU Numbering).

In some embodiments, the first Fc region comprises a mutation that decreases Fc receptor binding to the agent relative to an agent without the mutation.

In some embodiments, the first Fc region comprises an Asn297Ala (N297A) mutation or a Leu234Ala/Leu235Ala (LALA) mutation, or a combination thereof according to EU Numbering.

In some embodiments, the second Fc region comprises a mutation that decreases Fc receptor binding to the agent relative to an agent without the mutation.

In some embodiments, the second Fc region comprises an Asn297Ala (N297A) mutation or a Leu234Ala/Leu235Ala (LALA) mutation, or a combination thereof according to EU Numbering.

In some embodiments, the first Fc region comprises a sequence having at least 75% sequence identity to any one sequence selected from the group consisting of the sequence of SEQ ID NO: 73, the sequence of SEQ ID NO: 76, the sequence of SEQ ID NO: 74, the sequence of SEQ ID NO: 75, the sequence of SEQ ID NO: 40, the sequence of SEQ ID NO: 41, the sequence of SEQ ID NO: 42, the sequence of SEQ ID NO: 3645, the sequence of SEQ ID NO: 3646, the sequence of SEQ ID NO: 3649, the sequence of SEQ ID NO: 8687, the sequence of SEQ ID NO: 506, the sequence of SEQ ID NO: 507, the sequence of SEQ ID NO: 3647, the sequence of SEQ ID NO: 3648, the sequence of SEQ ID NO: 508 or 8686, the sequence of SEQ ID NO: 513, the sequence of SEQ ID NO: 514, the sequence of SEQ ID NO: 515, the sequence of SEQ ID NO: 516, the sequence of SEQ ID NO: 3447, and the sequence of SEQ ID NO: 511.

In some embodiments, the second Fc region comprises a sequence having at least 75% sequence identity to any one sequence selected from the group consisting of the sequence of SEQ ID NO: 73, the sequence of SEQ ID NO: 76, the sequence of SEQ ID NO: 74, the sequence of SEQ ID NO: 75, the sequence of SEQ ID NO: 40, the sequence of SEQ ID NO: 41, the sequence of SEQ ID NO: 42, the sequence of SEQ ID NO: 3645, the sequence of SEQ ID NO: 3646, the sequence of SEQ ID NO: 3649, the sequence of SEQ ID NO: 8687, the sequence of SEQ ID NO: 506, the sequence of SEQ ID NO: 507, the sequence of SEQ ID NO: 3647, the sequence of SEQ ID NO: 3648, the sequence of SEQ ID NO: 508 or 8686, the sequence of SEQ ID NO: 513, the sequence of SEQ ID NO: 514, the sequence of SEQ ID NO: 515, the sequence of SEQ ID NO: 516, the sequence of SEQ ID NO: 3447, and the sequence of SEQ ID NO: 511.

In some embodiments, the first Fc region comprises any one sequence selected from the group consisting of the sequence of SEQ ID NO: 73, the sequence of SEQ ID NO: 76, the sequence of SEQ ID NO: 74, the sequence of SEQ ID NO: 75, the sequence of SEQ ID NO: 40, the sequence of SEQ ID NO: 41, the sequence of SEQ ID NO: 42, the sequence of SEQ ID NO: 3645, the sequence of SEQ ID NO: 3646, the sequence of SEQ ID NO: 3649, the sequence of SEQ ID NO: 8687, the sequence of SEQ ID NO: 506, the sequence of SEQ ID NO: 507, the sequence of SEQ ID NO: 3647, the sequence of SEQ ID NO: 3648, the sequence of SEQ ID NO: 508 or 8686, the sequence of SEQ ID NO: 513, the sequence of SEQ ID NO: 514, the sequence of SEQ ID NO: 515, the sequence of SEQ ID NO: 516, the sequence of SEQ ID NO: 3447, and the sequence of SEQ ID NO: 511.

In some embodiments, the second Fc region comprises any one sequence selected from the group consisting of the sequence of SEQ ID NO: 73, the sequence of SEQ ID NO: 76, the sequence of SEQ ID NO: 74, the sequence of SEQ ID NO: 75, the sequence of SEQ ID NO: 40, the sequence of SEQ ID NO: 41, the sequence of SEQ ID NO: 42, the sequence of SEQ ID NO: 3645, the sequence of SEQ ID NO: 3646, the sequence of SEQ ID NO: 3649, the sequence of SEQ ID NO: 8687, the sequence of SEQ ID NO: 506, the sequence of SEQ ID NO: 507, the sequence of SEQ ID NO: 3647, the sequence of SEQ ID NO: 3648, the sequence of SEQ ID NO: 508 or 8686, the sequence of SEQ ID NO: 513, the sequence of SEQ ID NO: 514, the sequence of SEQ ID NO: 515, the sequence of SEQ ID NO: 516, the sequence of SEQ ID NO: 3447, and the sequence of SEQ ID NO: 511.

In some embodiments, the first Fc region comprises a sequence having at least 75% sequence identity to any one sequence selected from the group consisting of the sequence of SEQ ID NO: 3648, the sequence of SEQ ID NO: 508 or 8686, the sequence of SEQ ID NO: 506, the sequence of SEQ ID NO: 507, the sequence of SEQ ID NO: 3477, the sequence of SEQ ID NO: 511, the sequence of SEQ ID NO: 515, and the sequence of SEQ ID NO: 516.

In some embodiments, the second Fc region comprises a sequence having at least 75% sequence identity to the sequence of SEQ ID NO: 3648, the sequence of SEQ ID NO: 508 or 8686, the sequence of SEQ ID NO: 506, the sequence of SEQ ID NO: 507, the sequence of SEQ ID NO: 3477, the sequence of SEQ ID NO: 511, the sequence of SEQ ID NO: 515, or the sequence of SEQ ID NO: 516.

In some embodiments, the first Fc region comprises any one sequence selected from the group consisting of the sequence of SEQ ID NO: 3648, SEQ ID NO: 508 or 8686, SEQ ID NO: 506, SEQ ID NO: 507, SEQ ID NO: 3477, SEQ ID NO: 511, SEQ ID NO: 515, or SEQ ID NO: 516.

In some embodiments, the second Fc region comprises any one sequence selected from the group consisting of the sequence of SEQ ID NO: 3648, SEQ ID NO: 508 or 8686, SEQ ID NO: 506, SEQ ID NO: 507, SEQ ID NO: 3477, SEQ ID NO: 511, SEQ ID NO: 515, or SEQ ID NO: 516.

In some embodiments,
(i) the first Fc region comprises the sequence of SEQ ID NO: 506 or the sequence of SEQ ID NO: 507, and the second Fc region comprises the sequence of SEQ ID NO: 3648 or the sequence of SEQ ID NO: 508 or 8686; or
(ii) the first Fc region comprises the sequence of SEQ ID NO: 3447 or the sequence of SEQ ID NO: 511, and the second Fc region comprises the sequence of SEQ ID NO: 515 or the sequence of SEQ ID NO: 516.

In some embodiments,
(i) the first Fc region comprises the sequence of SEQ ID NO: 3648 or the sequence of SEQ ID NO: 508 or 8686, and the second Fc region comprises the sequence of SEQ ID NO: 506 or the sequence of SEQ ID NO: 507; or
(ii) the first Fc region comprises the sequence of SEQ ID NO: 515 or the sequence of SEQ ID NO: 516, and the second Fc region comprises the sequence of SEQ ID NO: 3447 or the sequence of SEQ ID NO: 511.

In some embodiments, the agent comprises a first polypeptide, a second polypeptide, and at least one cytokine molecule or a functional fragment or variant thereof, wherein the first polypeptide and the second polypeptide are non-contiguous;
wherein:
(i) the first polypeptide comprises a first member of the dimerization module linked to:
(a) the antigen binding domain comprising a VH and a VL, or
(b) a first portion of the antigen binding domain comprising the VH, wherein the agent further comprises a third polypeptide comprising a second portion of the antigen binding domain comprising a VL, wherein the third polypeptide is non-contiguous with the first polypeptide and the second polypeptide; and
(ii) the second polypeptide comprises a second member of the dimerization module, and
wherein the at least one cytokine molecule or a functional fragment or variant thereof is covalently linked to the second polypeptide or the first polypeptide.

In some embodiments, the agent comprises a first polypeptide, a second polypeptide, and at least one cytokine molecule or a functional fragment or variant thereof, wherein the first polypeptide and the second polypeptide are non-contiguous;
wherein:
(i) the first polypeptide comprises a first member of a dimerization module linked to a first portion of the antigen binding domain comprising the VH, wherein the agent further comprises a third polypeptide comprising a second portion of the antigen binding domain comprising the VL, wherein the third polypeptide is non-contiguous with the first polypeptide and the second polypeptide; and
(ii) the second polypeptide comprises a second member of the dimerization module, and wherein the at least one cytokine molecule or a functional fragment or variant thereof is covalently linked to the second polypeptide.

In some embodiments, the at least one cytokine molecule or a functional fragment or variant thereof is covalently linked to the N-terminus of the second polypeptide, the C-terminus of the second polypeptide, or a combination thereof.

In some embodiments, the at least one cytokine molecule or a functional fragment or variant thereof is within a single contiguous polypeptide chain of the second polypeptide.

In some embodiments, the first polypeptide comprises:
(i) the antigen binding domain comprising the VH and the VL, wherein the antigen binding domain further comprises a heavy chain constant domain 1 (CH1) linked to the VH; or
(ii) the first portion of the first antigen binding domain comprising the VH, wherein the first portion of the antigen binding domain further comprises a CH1 linked to the VH.

In some embodiments, the CH1 is linked to the C-terminus of the VH.

In some embodiments, the agent comprises:
(i) the first polypeptide comprising the antigen binding domain that comprises the VH and the VL, wherein the antigen binding domain further comprises a light chain constant domain (CL) linked to the VL; or
(ii) the first polypeptide comprising the first portion of the antigen binding domain and the third polypeptide comprising the second portion of the antigen binding domain, wherein the second portion of the antigen binding domain further comprises a CL linked to the VL.

In some embodiments, the CL is linked to the C-terminus of the VL.

In some embodiments, the first member of the dimerization module is linked to the C-terminus of the antigen binding domain comprising the VH and the VL, or the C-terminus of the first portion of the first antigen binding domain comprising the VH.

In some embodiments, the second polypeptide comprises:
(i) an antigen binding domain that binds TRBC, an second antigen binding domain that binds to NKp30, an immune cell engager, or an tumor-targeting moiety comprising a VH and a VL, wherein the antigen binding domain that binds TRBC, the second antigen binding domain that binds to NKp30, the immune cell engager, or the tumor-targeting moiety further comprises a heavy chain constant domain 1 (CH1) linked to the VH; or
(ii) the first portion of the antigen binding domain that binds TRBC, the second antigen binding domain that binds to NKp30, the immune cell engager, or the tumor-targeting moiety comprising the VH, wherein the first portion of the antigen binding domain that binds TRBC, the second antigen binding domain that binds to NKp30, the immune cell engager, or the tumor-targeting moiety further comprises a CH1 linked to the VH.

In some embodiments, the CH1 is linked to the C-terminus of the VH.

In some embodiments, the agent comprises:
(i) the second polypeptide comprising an antigen binding domain that binds TRBC, an second antigen binding domain that binds to NKp30, an immune cell engager, or an tumor-targeting moiety that comprises a VH and a VL, wherein the antigen binding domain that binds TRBC, the second antigen binding domain that binds to NKp30, the immune cell engager, or the tumor-targeting moiety further comprises a light chain constant domain (CL) linked to the VL; or
(ii) the second polypeptide comprising the first portion of the antigen binding domain that binds TRBC, the second antigen binding domain that binds to NKp30, the immune cell engager, or the tumor-targeting moiety,
wherein the agent further comprises a fourth polypeptide comprising the second portion of the antigen binding domain that binds TRBC, the second antigen binding domain that binds to NKp30, the immune cell engager, or the tumor-targeting moiety, wherein the fourth polypeptide is non-contiguous with the first polypeptide, the second polypeptide, and third polypeptide, and
wherein the second portion of the antigen binding domain that binds TRBC, the second antigen binding domain that binds to NKp30, the immune cell engager, or the tumor-targeting moiety further comprises a CL linked to the VL.

In some embodiments, the CL is linked to the C-terminus of the VL.

In some embodiments, the second member of the dimerization module is linked to the C-terminus of the antigen binding domain that binds TRBC, the second antigen binding domain that binds to NKp30, the immune cell engager, or the tumor-targeting moiety comprising the VH and the VL, or the C-terminus of the first portion of the antigen binding domain that binds TRBC, the second antigen binding domain that binds to NKp30, the immune cell engager, or the tumor-targeting moiety comprising the VH.

In some embodiments, the agent further comprises:
(i) a linker between the first member of the dimerization module and the antigen binding domain comprising a VH and a VL, or the first portion of the antigen binding domain comprising a VH;
(ii) a linker between the second member of the dimerization module and an antigen binding domain that binds TRBC, an second antigen binding domain that binds to NKp30, an immune cell engager, or an tumor-targeting moiety comprising a VH and a VL, or the first portion of the antigen binding domain that binds TRBC, the second antigen binding domain that binds to NKp30, the immune cell engager, or the tumor-targeting moiety comprising a VH;
(iii) a linker between the VH and the VL of the antigen binding domain;
(iv) a linker between the CH1 and the VH of the antigen binding domain;
(v) a linker between the CL and the VL of the antigen binding domain;
(vi) a linker between the VH and the VL of the antigen binding domain that binds TRBC, the second antigen binding domain that binds to NKp30, the immune cell engager, or the tumor-targeting moiety;
(vii) a linker between the CH1 and the VH of the antigen binding domain that binds TRBC, the second antigen binding domain that binds to NKp30, the immune cell engager, or the tumor-targeting moiety;
(viii) a linker between the CL and the VL of the antigen binding domain that binds TRBC, the second antigen binding domain that binds to NKp30, the immune cell engager, or the tumor-targeting moiety;
(ix) a linker between the at least one cytokine molecule or a functional fragment or variant thereof and the first polypeptide, a linker between the at least one cytokine molecule or a functional fragment or variant thereof and the second polypeptide, or any combination thereof; or
(x) any combination thereof.

In some embodiments, the linker is selected from the group consisting of a cleavable linker, a non-cleavable linker, a peptide linker, a flexible linker, a rigid linker, a helical linker, and a non-helical linker.

In some embodiments, the linker is the peptide linker comprises the sequence of SEQ ID NO: 3473, SEQ ID NO: 3308, or SEQ ID NO: 8291.

In some embodiments, the at least one cytokine molecule or a functional fragment or variant thereof is linked to the N-terminus of the first polypeptide, the C-terminus of the first polypeptide, the N-terminus of the second polypeptide, the C-terminus of the second polypeptide, the N-terminus of the third polypeptide, the C-terminus of the third polypeptide, or any combination thereof.

In some embodiments, the at least one cytokine molecule or a functional fragment or variant thereof is within a single contiguous polypeptide chain of the first polypeptide, the second polypeptide, or the third cytokine polypeptide to which the at least one cytokine molecule or a functional fragment or variant thereof is linked.

In some embodiments, the agent comprises:
(i) the first polypeptide comprising the first portion of the dimerization module linked to the C-terminus of the first portion of the antigen binding domain;
(ii) the second polypeptide comprising the second portion of the dimerization module;
(iii) the third polypeptide comprising the second portion of the antigen binding domain; and
(iv) the at least one cytokine molecule or a functional fragment or variant thereof covalently linked to the N-terminus of the second polypeptide.

In some embodiments, the agent comprises:
(i) a first polypeptide chain comprising the sequence of SEQ ID NO: 505, and the sequence of SEQ ID NO: 3649 or SEQ ID NO: 3451;
(ii) a second polypeptide chain comprising the sequence of SEQ ID NO: 3472, the sequence of SEQ ID NO: 2170, and the sequence of SEQ ID NO: 3648 or SEQ ID NO: 508 or 8686; and
(iii) a third polypeptide comprising the sequence of SEQ ID NO: 13 and the sequence of SEQ ID NO: 3644.

In some embodiments, the agent comprises:
(i) a first polypeptide chain comprising, from the N-terminus to the C-terminus, the sequence of SEQ ID NO: 505 operatively linked to the sequence of SEQ ID NO: 3649 or SEQ ID NO: 3451;
(ii) a second polypeptide chain comprising, from the N-terminus to the C-terminus, the sequence of SEQ ID NO: 3472 operatively linked to the sequence of SEQ ID NO: 2170 operatively linked to the sequence of SEQ ID NO: 3648 or SEQ ID NO: 508 or 8686; and
(iii) a third polypeptide comprising, from the N-terminus to the C-terminus, the sequence of SEQ ID NO: 13 operatively linked to the sequence of SEQ ID NO: 3644.

In some embodiments, the second polypeptide chain comprises, from the N-terminus to the C-terminus, the sequence of SEQ ID NO: 3472 operatively linked to the sequence of SEQ ID NO: 3473 operatively linked to the sequence of SEQ ID NO: 2170 operatively linked to the sequence of SEQ ID NO: 3308 operatively linked to the sequence of SEQ ID NO: 3648 or SEQ ID NO: 508 or 8686.

In some embodiments, the agent comprises:
(i) a first polypeptide chain comprising the sequence of SEQ ID NO: 505, and the sequence of SEQ ID NO: 3649 or SEQ ID NO: 3451;
(ii) a second polypeptide chain comprising the sequence of SEQ ID NO: 3474, and the sequence of SEQ ID NO: 3648 or SEQ ID NO: 508 or 8686; and
(iii) a third polypeptide comprising the sequence of SEQ ID NO: 13 and the sequence of SEQ ID NO: 3644.

In some embodiments, the agent comprises:
(i) a first polypeptide chain comprising, from the N-terminus to the C-terminus, the sequence of SEQ ID NO: 505 operatively linked to the sequence of SEQ ID NO: 3649 or SEQ ID NO: 3451;
(ii) a second polypeptide chain comprising, from the N-terminus to the C-terminus, the sequence of SEQ ID NO: 3474 operatively linked to the sequence of SEQ ID NO: 3648 or SEQ ID NO: 508 or 8686; and
(iii) a third polypeptide comprising, from the N-terminus to the C-terminus, the sequence of SEQ ID NO: 13 operatively linked to the sequence of SEQ ID NO: 3644.

In some embodiments, the second polypeptide comprises, from the N-terminus to the C-terminus, the sequence of SEQ ID NO: 3474 operatively linked to the sequence of SEQ ID NO: 3308 operatively linked to the sequence of SEQ ID NO: 3648 or SEQ ID NO: 508 or 8686.

In some embodiments, the agent comprises:
(i) a first polypeptide chain comprising a sequence having at least 75% sequence identity to the sequence of SEQ ID NO: 500;
(ii) a second polypeptide chain comprising a sequence having at least 75% sequence identity to the sequence of SEQ ID NO: 3420; and
(iii) a third polypeptide comprising a sequence having at least 75% sequence identity to the sequence of SEQ ID NO: 501.

In some embodiments, the agent comprises:
(i) a first polypeptide chain comprising a sequence having at least 75% sequence identity to the sequence of SEQ ID NO: 4702;
(ii) a second polypeptide chain comprising a sequence having at least 75% sequence identity to the sequence of SEQ ID NO: 4703; and
(iii) a third polypeptide comprising a sequence having at least 75% sequence identity to the sequence of SEQ ID NO: 501.

In some embodiments, the agent comprises:
(i) a first polypeptide chain comprising the sequence of SEQ ID NO: 500;
(ii) a second polypeptide chain comprising the sequence of SEQ ID NO: 3420; and
(iii) a third polypeptide comprising the sequence of SEQ ID NO: 501.

In some embodiments, the agent comprises:
(i) a first polypeptide chain comprising the sequence of SEQ ID NO: 4702;
(ii) a second polypeptide chain comprising the sequence of SEQ ID NO: 3420; and
(iii) a third polypeptide comprising the sequence of SEQ ID NO: 4703. In some embodiments, the agent comprises:
(i) a first polypeptide chain comprising the sequence of SEQ ID NO: 505, and the sequence of SEQ ID NO: 3649 or SEQ ID NO: 3451;
(ii) a second polypeptide chain comprising the sequence of SEQ ID NO: 3472, the sequence of SEQ ID NO: 3670, and the sequence of SEQ ID NO: 3648 or SEQ ID NO: 508 or 8686; and
(iii) a third polypeptide comprising the sequence of SEQ ID NO: 13 and the sequence of SEQ ID NO: 3644.

In some embodiments, the agent comprises:
(i) a first polypeptide chain comprising, from the N-terminus to the C-terminus, the sequence of SEQ ID NO: 505 operatively linked to the sequence of SEQ ID NO: 3649 or SEQ ID NO: 3451;
(ii) a second polypeptide chain comprising, from the N-terminus to the C-terminus, the sequence of SEQ ID NO: 3472 operatively linked to the sequence of SEQ ID NO: 3670 operatively linked to the sequence of SEQ ID NO: 3648 or SEQ ID NO: 508 or 8686; and
(iii) a third polypeptide comprising, from the N-terminus to the C-terminus, the sequence of SEQ ID NO: 13 operatively linked to the sequence of SEQ ID NO: 3644.

In some embodiments, the second polypeptide comprises, from the N-terminus to the C-terminus, the sequence of SEQ ID NO: 3472 operatively linked to the sequence of SEQ ID NO: 3473 operatively linked to the sequence of SEQ ID NO: 3670 operatively linked to the sequence of SEQ ID NO: 3308 operatively linked to the sequence of SEQ ID NO: 3648 or SEQ ID NO: 508 or 8686.

In some embodiments, the agent comprises:
(i) a first polypeptide chain comprising the sequence of SEQ ID NO: 505, and the sequence of SEQ ID NO: 3649 or SEQ ID NO: 3451;
(ii) a second polypeptide chain comprising the sequence of SEQ ID NO: 3671, and the sequence of SEQ ID NO: 3648 or SEQ ID NO: 508 or 8686; and
(iii) a third polypeptide comprising the sequence of SEQ ID NO: 13 and the sequence of SEQ ID NO: 3644.

In some embodiments, the agent comprises:
(i) a first polypeptide chain comprising, from the N-terminus to the C-terminus the sequence of SEQ ID NO: 505 operatively linked to the sequence of SEQ ID NO: 3649 or SEQ ID NO: 3451;
(ii) a second polypeptide chain comprising, from the N-terminus to the C-terminus, the sequence of SEQ ID NO: 3671 operatively linked to the sequence of SEQ ID NO: 3648 or SEQ ID NO: 508 or 8686;
(iii) a third polypeptide comprising, from the N-terminus to the C-terminus, comprising the sequence of SEQ ID NO: 13, and the sequence of SEQ ID NO: 3644.

In some embodiments, the second polypeptide comprises, from the N-terminus to the C-terminus, the sequence of SEQ ID NO: 3671 operatively linked to the sequence of SEQ ID NO: 3308 operatively linked to the sequence of SEQ ID NO: 3648 or SEQ ID NO: 508 or 8686.

In some embodiments, the wherein the agent comprises:
(i) a first polypeptide chain comprising a sequence having at least 75% sequence identity to the sequence of SEQ ID NO: 500;
(ii) a second polypeptide chain comprising a sequence having at least 75% sequence identity to the sequence of SEQ ID NO: 3669; and
(iii) a third polypeptide comprising a sequence having at least 75% sequence identity to the sequence of SEQ ID NO: 501.

In some embodiments, the wherein the agent comprises:
(i) a first polypeptide chain comprising a sequence having at least 75% sequence identity to the sequence of SEQ ID NO: 4702;
(ii) a second polypeptide chain comprising a sequence having at least 75% sequence identity to the sequence of SEQ ID NO: 4708; and
(iii) a third polypeptide comprising a sequence having at least 75% sequence identity to the sequence of SEQ ID NO: 501.

In some embodiments, the wherein the agent comprises:
(i) a first polypeptide chain comprising the sequence of SEQ ID NO: 500;
(ii) a second polypeptide chain comprising the sequence of SEQ ID NO: 3669; and
(iii) a third polypeptide comprising the sequence of SEQ ID NO: 501.

In some embodiments, the wherein the agent comprises:
(i) a first polypeptide chain comprising the sequence of SEQ ID NO: 4702;
(ii) a second polypeptide chain comprising the sequence of SEQ ID NO: 4708; and
(iii) a third polypeptide comprising the sequence of SEQ ID NO: 501.

In some embodiments, the agent comprises:
(i) a first polypeptide chain comprising the sequence of SEQ ID NO: 505, and the sequence of SEQ ID NO: 3649 or SEQ ID NO: 3451;
(ii) a second polypeptide chain comprising the sequence of 2270, and the sequence of SEQ ID NO: 3648 or SEQ ID NO: 508 or 8686; and
(iii) a third polypeptide comprising the sequence of SEQ ID NO: 13 and the sequence of SEQ ID NO: 3644.

In some embodiments, the agent comprises:
(i) a first polypeptide chain comprising, from the N-terminus to the C-terminus, the sequence of SEQ ID NO: 505 operatively linked to the sequence of SEQ ID NO: 3649 or SEQ ID NO: 3451;
(ii) a second polypeptide chain comprising, from the N-terminus to the C-terminus, the sequence of 2270 operatively linked to the sequence of SEQ ID NO: 3648 or SEQ ID NO: 508 or 8686; and
(iii) a third polypeptide comprising the sequence of SEQ ID NO: 13 and the sequence of SEQ ID NO: 3644.

In some embodiments, the second polypeptide comprises from the N-terminus to the C-terminus, the sequence of SEQ ID NO: 2270 operatively linked to the sequence of SEQ ID NO: 3308 operatively linked to the sequence of SEQ ID NO: 3648 or SEQ ID NO: 508 or 8686.

In some embodiments, the agent comprises:
(i) a first polypeptide chain comprising a sequence having at least 75% sequence identity to the sequence of SEQ ID NO: 500;
(ii) a second polypeptide chain comprising a sequence having at least 75% sequence identity to the sequence of SEQ ID NO: 3340; and
(iii) a third polypeptide comprising a sequence having at least 75% sequence identity to the sequence of SEQ ID NO: 501.

In some embodiments, the agent comprises:
(i) a first polypeptide chain comprising a sequence having at least 75% sequence identity to the sequence of SEQ ID NO: 4702;
(ii) a second polypeptide chain comprising a sequence having at least 75% sequence identity to the sequence of SEQ ID NO: 4706; and
(iii) a third polypeptide comprising a sequence having at least 75% sequence identity to the sequence of SEQ ID NO: 501.

In some embodiments, the agent comprises:
(i) a first polypeptide chain comprising the sequence of SEQ ID NO: 500;
(ii) a second polypeptide chain comprising the sequence of SEQ ID NO: 3340; and
(iii) a third polypeptide comprising the sequence of SEQ ID NO: 501.

In some embodiments, the agent comprises:
(i) a first polypeptide chain comprising the sequence of SEQ ID NO: 4702;
(ii) a second polypeptide chain comprising the sequence of SEQ ID NO: 4706; and
(iii) a third polypeptide comprising the sequence of SEQ ID NO: 501.

In some embodiments, the agent comprises a first polypeptide and a second polypeptide, wherein the first polypeptide and the second polypeptide are non-contiguous, and wherein:
(i) the first polypeptide comprises a first member of the dimerization module linked to:
(a) the antigen binding domain comprising a VH of the antigen binding domain and a VL of the antigen binding domain, or
(b) a first portion of the antigen binding domain comprising the VH of the antigen binding domain, wherein the agent further comprises a third polypeptide comprising a second portion of the antigen binding domain comprising a VL of the antigen binding domain, wherein the third polypeptide is non-contiguous with the first polypeptide and the second polypeptide; and
(ii) the second polypeptide comprises a second member of the dimerization module linked to:
(a) an antigen binding domain that binds to T cell receptor beta chain constant domain (TRBC) comprising a VH and a VL, or
(b) a first portion of the antigen binding domain that binds to TRBC comprising the VH of the antigen binding domain that binds to TRBC, wherein the agent further comprises a fourth polypeptide comprising a second portion of the antigen binding domain that binds to TRBC comprising a VL of the antigen binding domain that binds to TRBC, wherein the fourth polypeptide is non-contiguous with the first polypeptide, the second polypeptide, and the third polypeptide.

In some embodiments, the agent comprises a first polypeptide and a second polypeptide, wherein the first polypeptide and the second polypeptide are non-contiguous, wherein:
(i) the first polypeptide comprises a first member of the dimerization module linked to a first portion of the antigen binding domain comprising the VH of the antigen binding domain, wherein the agent further comprises a third polypeptide comprising a second portion of the antigen binding domain comprising a VL of the antigen binding domain, wherein the third polypeptide is non-contiguous with the first polypeptide and the second polypeptide; and
(ii) the second polypeptide comprises a second member of the dimerization module linked to an antigen binding domain that binds to TRBC.

In some embodiments, the agent comprises:
(i) a first polypeptide chain comprising the sequence of SEQ ID NO: 505, the sequence of SEQ ID NO: 13 and the sequence of SEQ ID NO: 3447 or SEQ ID NO: 511;
(ii) a third polypeptide comprising the sequence of SEQ ID NO: 8308 and the sequence of SEQ ID NO: 3644; and
(iii) a second polypeptide chain comprising the sequence of SEQ ID NO: 517 and the sequence of SEQ ID NO: 513 or SEQ ID NO: 514; or a second polypeptide chain comprising the sequence of SEQ ID NO: 512 and the sequence of SEQ ID NO: 513 or SEQ ID NO: 514.

In some embodiments, the agent comprises:
(i) a first polypeptide chain comprising, from the N-terminus to the C-terminus, the sequence of SEQ ID NO: 505 operatively linked to the sequence of SEQ ID NO: 13 operatively linked to the sequence of SEQ ID NO: 3447 or SEQ ID NO: 511;
(ii) a third polypeptide comprising, from the N-terminus to the C-terminus, the sequence of SEQ ID NO: 8308 operatively linked to the sequence of SEQ ID NO: 3644; and
(iii) a second polypeptide chain comprising, from the N-terminus to the C-terminus, the sequence of SEQ ID NO: 517 operatively linked to the sequence of SEQ ID NO: 513 or SEQ ID NO: 514; or a second polypeptide chain comprising, from the N-terminus to the C-terminus, the sequence of SEQ ID NO: 512 operatively linked to the sequence of SEQ ID NO: 513 or SEQ ID NO: 514.

In some embodiments, the first polypeptide comprises, from the N-terminus to the C-terminus, the sequence of SEQ ID NO: 505 operatively linked to the sequence of SEQ ID NO: 8291 operatively linked to the sequence of SEQ ID NO: 13.

In some embodiments, the agent comprises:
(i) a first polypeptide chain comprising a sequence having at least 75% sequence identity to the sequence of SEQ ID NO: 509 or the sequence of SEQ ID NO: 4704;
(ii) a third polypeptide comprising a sequence having at least 75% sequence identity to the sequence of SEQ ID NO: 7380; and
(iii) a second polypeptide chain comprising a sequence having at least 75% sequence identity to the sequence of SEQ ID NO: 7382, the sequence of SEQ ID NO: 4677, the sequence of SEQ ID NO: 7379, or the sequence of 4675.

In some embodiments, the agent comprises:
(i) a first polypeptide chain comprising the sequence of SEQ ID NO: 509 or the sequence of SEQ ID NO: 4704;
(ii) a third polypeptide comprising the sequence of SEQ ID NO: 7380; and
(iii) a second polypeptide chain comprising the sequence of SEQ ID NO: 7382, the sequence of SEQ ID NO: 4677, the sequence of SEQ ID NO: 7379, or the sequence of SEQ ID NO: 4675.

In some embodiments, the agent comprises a first polypeptide chain comprising the sequence of SEQ ID NO: 505, the sequence of SEQ ID NO: 13 and the sequence of SEQ ID NO: 3447 or SEQ ID NO: 511.

In some embodiments, the agent comprises a first polypeptide chain comprising, from the N-terminus to the C-terminus, the sequence of SEQ ID NO: 505 operatively linked to the sequence of SEQ ID NO: 13 operatively linked to sequence of SEQ ID NO: 3447 or SEQ ID NO: 511.

In some embodiments, the first polypeptide comprises, from the N-terminus to the C-terminus, the sequence of SEQ ID NO: 505 operatively linked to the sequence of SEQ ID NO: 8291 operatively linked to the sequence of SEQ ID NO: 13.

In some embodiments, the agent comprises a first polypeptide chain comprising a sequence having at least 75% sequence identity to the sequence of SEQ ID NO: 509.

In some embodiments, the agent comprises a first polypeptide chain comprising the sequence of SEQ ID NO: 509.

In some embodiments, the agent comprises:
(i) a third polypeptide comprising the sequence of SEQ ID NO: 7419 and the sequence of SEQ ID NO: 3644; and
(ii) a second polypeptide chain comprising the sequence of SEQ ID NO: 7420 and the sequence of SEQ ID NO: 513 or SEQ ID NO: 514 or a second polypeptide chain comprising the sequence of SEQ ID NO: 7423 and the sequence of SEQ ID NO: 513 or SEQ ID NO: 514.

In some embodiments, the agent comprises:
(i) a third polypeptide comprising, from the N-terminus to the C-terminus, the sequence of SEQ ID NO: 7419 operatively linked to the sequence of SEQ ID NO: 3644; and
(ii) a second polypeptide chain comprising, from the N-terminus to the C-terminus, the sequence of SEQ ID NO: 7420 operatively linked to the sequence of SEQ ID NO: 513 or SEQ ID NO: 514 or a second polypeptide chain comprising the sequence of SEQ ID NO: 7423 operatively linked to the sequence of SEQ ID NO: 513 or SEQ ID NO: 514.

In some embodiments, the agent comprises:
(i) a third polypeptide comprising a sequence having at least 75% sequence identity to the sequence of SEQ ID NO: 7439; and
(ii) a second polypeptide chain comprising a sequence having at least 75% sequence identity to the sequence of SEQ ID NO: 4680, the sequence of SEQ ID NO: 7438, the sequence of SEQ ID NO: 4681, or the sequence of SEQ ID NO: 7440.

In some embodiments, the agent comprises:
(i) a third polypeptide comprising the sequence of SEQ ID NO: 7439; and
(ii) a second polypeptide chain comprising the sequence of SEQ ID NO: 4680, the sequence of SEQ ID NO: 7438, the sequence of SEQ ID NO: 4681, or the sequence of SEQ ID NO: 7440.

In some embodiments, the agent comprises:
(i) a third polypeptide comprising the sequence of SEQ ID NO: 8012 and the sequence of SEQ ID NO: 3644; and
(ii) a second polypeptide chain comprising the sequence of SEQ ID NO: 8011 and the sequence of SEQ ID NO: 513 or SEQ ID NO: 514.

In some embodiments, the agent comprises:
(i) a third polypeptide comprising, from the N-terminus to the C-terminus, the sequence of SEQ ID NO: 8012 operatively linked to the sequence of SEQ ID NO: 3644; and
(ii) a second polypeptide chain comprising, from the N-terminus to the C-terminus, the sequence of SEQ ID NO: 8011 operatively linked to the sequence of SEQ ID NO: 513 or SEQ ID NO: 514.

In some embodiments, the agent comprises:
(i) a third polypeptide comprising a sequence having at least 75% sequence identity to the sequence of SEQ ID NO: 8002; and
(ii) a second polypeptide chain comprising a sequence having at least 75% sequence identity to the sequence of SEQ ID NO: 8001 or 8697.

In some embodiments, the agent comprises:
(i) a third polypeptide comprising a sequence having at least 75% sequence identity to the sequence of SEQ ID NO: 8002; and
(ii) a second polypeptide chain comprising a sequence having at least 75% sequence identity to the sequence of SEQ ID NO: 4682.

In some embodiments, the agent comprises:
(i) a third polypeptide comprising the sequence of SEQ ID NO: 8002; and
(ii) a second polypeptide chain comprising the sequence of SEQ ID NO: 8001 or 8697.

In some embodiments, the agent comprises:
(i) a third polypeptide comprising the sequence of SEQ ID NO: 8002; and
(ii) a second polypeptide chain comprising the sequence of SEQ ID NO: 4682.

In some embodiments, the agent comprises:
(i) a third polypeptide comprising the sequence of SEQ ID NO: 8014 and the sequence of SEQ ID NO: 3644; and
(ii) a second polypeptide chain comprising the sequence of SEQ ID NO: 8013 and the sequence of SEQ ID NO: 513 or SEQ ID NO: 514, a second polypeptide chain comprising the sequence of SEQ ID NO: 8299 and the sequence of SEQ ID NO: 513 or SEQ ID NO: 514, or a second polypeptide chain comprising the sequence of SEQ ID NO: 8303 and the sequence of SEQ ID NO: 513 or SEQ ID NO: 514.

In some embodiments, the agent comprises:
(i) a third polypeptide comprising, from the N-terminus to the C-terminus, the sequence of SEQ ID NO: 8014 operatively linked to the sequence of SEQ ID NO: 3644; and
(ii) a second polypeptide chain comprising, from the N-terminus to the C-terminus, the sequence of SEQ ID NO: 8013 operatively linked to the sequence of SEQ ID NO: 513 or SEQ ID NO: 514, a second polypeptide chain comprising the sequence of SEQ ID NO: 8299 operatively linked to the sequence of SEQ ID NO: 513 or SEQ ID NO: 514, or a second polypeptide chain comprising the sequence of SEQ ID NO: 8303 operatively linked to the sequence of SEQ ID NO: 513 or SEQ ID NO: 514.

In some embodiments, the agent comprises:
(i) a third polypeptide comprising a sequence having at least 75% sequence identity to the sequence of SEQ ID NO: 8005; and
(ii) a second polypeptide chain comprising a sequence having at least 75% sequence identity to the sequence of SEQ ID NO: 8004, the sequence of SEQ ID NO: 4685, the sequence of SEQ ID NO: 8007, the sequence of SEQ ID NO: 4686, the sequence of SEQ ID NO: 4687, or the sequence of SEQ ID NO: 8009.

In some embodiments, the agent comprises:
(i) a third polypeptide comprising the sequence of SEQ ID NO: 8005; and
(ii) a second polypeptide chain comprising the sequence of SEQ ID NO: 8004, the sequence of SEQ ID NO: 4685, the sequence of SEQ ID NO: 8007, the sequence of SEQ ID NO: 4686, the sequence of SEQ ID NO: 4687, or the sequence of SEQ ID NO: 8009.

In some embodiments, the agent further comprises at least one cytokine molecule or a functional fragment or variant thereof, wherein the at least one cytokine molecule or a functional fragment or variant thereof is linked to the first member of the dimerization module, the second member of the dimerization module, the antigen binding domain, the antigen binding domain that binds to TRBC, or any combination thereof.

In some embodiments, the at least one cytokine molecule or a functional fragment or variant thereof comprises IL-2 or a functional fragment or variant thereof, IL-7 or a functional fragment or variant thereof, IL-12 or a functional fragment or variant thereof, IL-15 or a functional fragment or variant thereof, IL-18 or a functional fragment or variant thereof, IL-21 or a functional fragment or variant thereof, interferon gamma or a functional fragment or variant thereof, or any combination thereof.

In some embodiments, the at least one cytokine molecule or a functional fragment or variant thereof comprises a sequence having at least 75% sequence identity to any one sequence selected from the group consisting of the cytokine sequences listed in Tables 40, 42C, 43A, 43B, and 46-51.

In some embodiments, the at least one cytokine molecule or a functional fragment or variant thereof comprises any one sequence selected from the group consisting of the cytokine sequences listed in Tables 40, 42C, 43A, 43B, and 46-51.

In some embodiments, the at least one cytokine molecule or a functional fragment or variant thereof comprises IL-2 or a functional fragment or variant thereof.

In some embodiments, the IL-2 or a functional fragment or variant thereof comprises a sequence having at least 75% sequence identity to the sequence of SEQ ID NO: 2191, the sequence of SEQ ID NO: 2270, or the sequence of SEQ ID NO: 2280.

In some embodiments, the IL-2 or a functional fragment or variant thereof comprises a sequence the sequence of SEQ ID NO: 2191, the sequence of SEQ ID NO: 2270, or the sequence of SEQ ID NO: 2280.

In some embodiments, the at least one cytokine molecule or a functional fragment or variant thereof IL-15 or a functional fragment or variant thereof.

In some embodiments, the IL-15 or a functional fragment or variant thereof comprises a sequence having at least 75% sequence identity to the sequence of SEQ ID NO: 2170, SEQ ID NO: 3670, or SEQ ID NO: 3673.

In some embodiments, the IL-15 or a functional fragment or variant thereof comprises the sequence of SEQ ID NO: 2170, the sequence of SEQ ID NO: 3670, or the sequence of SEQ ID NO: 3673.

In some embodiments, the at least one cytokine molecule or a functional fragment or variant thereof further comprises an IL15Ralpha dimerizing domain covalently linked the IL-15 or a functional fragment or variant thereof.

In some embodiments, the IL15Ralpha dimerizing domain comprises an IL-15 receptor alpha sushi domain or a functional fragment or variant thereof.

In some embodiments, the IL15Ralpha dimerizing domain comprises a sequence having at least 75% sequence identity to the sequence of SEQ ID NO: 3472.

In some embodiments, the IL15Ralpha dimerizing domain comprises the sequence of SEQ ID NO: 3472.

In some embodiments, the IL-15 or a functional fragment or variant thereof is covalently linked to the IL15Ralpha dimerizing domain via a linker.

In some embodiments, the IL-15 or a functional fragment or variant thereof is covalently linked to the IL15Ralpha dimerizing domain via a linker comprising the sequence of SEQ ID NO: 3473.

In some embodiments, the at least one cytokine molecule or a functional fragment or variant thereof comprises a sequence having at least 75% sequence identity to the sequence of SEQ ID NO: 3474, SEQ ID NO: 3671, or SEQ ID NO: 8689.

In some embodiments, the at least one cytokine molecule or a functional fragment or variant thereof comprises the sequence of SEQ ID NO: 3474, the sequence of SEQ ID NO: 3671, or the sequence of SEQ ID NO: 8689.

In some embodiments, the at least one cytokine molecule or a functional fragment or variant thereof comprises IL-7 or a functional fragment or variant thereof.

In some embodiments, the IL-7 or the functional fragment or variant thereof comprises a sequence having at least 75% sequence identity to the sequence of SEQ ID NO: 3540.

In some embodiments, the IL-7 or the functional fragment or variant thereof comprises the sequence of SEQ ID NO: 3540.

In some embodiments, the at least one cytokine molecule or a functional fragment or variant thereof comprises IL-12 or a functional fragment or variant or variant thereof.

In some embodiments, the IL-12 or the functional fragment or variant thereof comprises a sequence having at least 75% sequence identity to the sequence of SEQ ID NO: 3542, the sequence of SEQ ID NO: 3543, or the sequence of SEQ ID NO: 3545.

In some embodiments, the IL-12 or the functional fragment or variant thereof comprises the sequence of SEQ ID NO: 3542, the sequence of SEQ ID NO: 3543, or the sequence of SEQ ID NO: 3545.

In some embodiments, the at least one cytokine molecule or a functional fragment or variant thereof comprises IL-18 of a functional fragment or variant thereof.

In some embodiments, the IL-18 or the functional fragment or variant thereof comprises a sequence having at least 75% sequence identity to the sequence of SEQ ID NO: 2192.

In some embodiments, the IL-18 or the functional fragment or variant thereof comprises the sequence of SEQ ID NO: 2192.

In some embodiments, the at least one cytokine molecule or a functional fragment or variant thereof comprises IL-21 or a functional fragment or variant thereof.

In some embodiments, the IL-21 or the functional fragment or variant thereof comprises a sequence having at least 75% sequence identity to the sequence of SEQ ID NO: 3540.

In some embodiments, the IL-21 or the functional fragment or variant thereof comprises the sequence of SEQ ID NO: 3540.

In another aspect, provided herein is a recombinant polynucleotide molecule comprising a polynucleotide sequence encoding the agent of the composition as provided herein.

In some embodiments, the recombinant polynucleotide molecule is an isolated recombinant polynucleotide molecule.

In another aspect, provided herein is a vector comprising the recombinant polynucleotide molecule as provided herein.

In another aspect, provided herein is a cell comprising the recombinant polynucleotide molecule as provided herein, or the vector as provided herein.

In another aspect, provided herein is a method of making the agent of the composition of any one as provided herein, comprising culturing the cell as provided herein under conditions suitable for expression of the agent.

In another aspect, provided herein is a pharmaceutical composition comprising the composition as provided herein, the recombinant polynucleotide molecule as provided herein, the vector as provided herein, or the cell as provided herein, and a pharmaceutically acceptable carrier, excipient, or diluent.

In another aspect, provided herein is a method of treating a condition or disease in a subject in need thereof comprising administering to the subject a therapeutically effective amount of the composition as provided herein, the recombinant polynucleotide molecule as provided herein, the vector as provided herein, the cell as provided herein, the pharmaceutical composition as provided herein, or any combination thereof, thereby treating the condition or disease in the subject.

In some embodiments, the condition or disease is cancer.

In some embodiments, the cancer is leukemia or lymphoma.

In some embodiments, the cancer is selected from the group consisting of acquired immune deficiency syndrome (AIDS)-associated lymphoma, Angioimmunoblastic T-cell lymphoma, Adult T-cell leukemia/lymphoma, Burkitt lymphoma, Central nervous system (CNS) lymphoma, Diffuse large B-cell lymphoma (DLBCL), Lymphoblastic lymphoma, Mantle cell lymphoma (MCL), Peripheral T-cell lymphoma (PTCL), Transformed follicular and transformed mucosa-associated lymphoid tissue (MALT) lymphomas, Cutaneous T-cell lymphoma (mycosis fungoides and Sézary syndrome), Follicular lymphoma, Lymphoplasmacytic lymphoma/Waldenström macroglobulinemia, Marginal zone B-cell lymphoma, Gastric mucosa-associated lymphoid tissue (MALT) lymphoma, Chronic lymphocytic leukemia/small-cell lymphocytic lymphoma (CLL/SLL), Extranodal T-/NK-cell lymphoma, and Anaplastic large-cell lymphoma.

In some embodiments, peripheral T-cell lymphoma (PTCL) is Hepatosplenic T-cell lymphoma (HSGDTCL), Subcutaneous paniculitis-like T-cell lymphoma, or Enteropathy-associated T-cell lymphoma.

In some embodiments, the cutaneous T-cell lymphoma is mycosis fungoides and Sdzary syndrome.

In some embodiments, the extranodal T-/NK-cell lymphoma is a nasal type.

In some embodiments, the anaplastic large-cell lymphoma is primary cutaneous anaplastic large-cell lymphoma or systemic anaplastic large-cell lymphoma.

In some embodiments, the cancer is a solid tumor.

In some embodiments, the solid tumor is selected from the group consisting of pancreatic cancer, breast cancer, colon cancer, colorectal cancer, lung cancer, skin cancer, ovarian cancer, liver cancer, esophageal cancer, endometrial cancer, gastric cancer, head and neck cancer, kidney cancer, and prostate cancer.

In some embodiments, the pancreatic cancer is pancreatic adenocarcinoma or pancreatic ductal adenocarcinoma.

In some embodiments, the lung cancer is small cell lung cancer (SCLC) or non-small cell lung cancer (NSCLC).

In another aspect, provided herein is a method of expanding and/or activating NK cells in a NK cell population comprising contacting the NK cell population with an effective amount of the composition as provided herein, the recombinant polynucleotide molecule as provided herein, the vector as provided herein, the cell as provided herein, the pharmaceutical composition as provided herein, or any combination thereof, thereby, expanding and/or activating the NK cells in the NK cell population.

In some embodiments, the expanding and/or activating NK cells is in vivo or ex vivo expansion and/or activation.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1D are schematic representations of exemplary formats and configurations of the agents as provided herein. In some embodiments, the agent comprises the antigen binding domain that bind to NKp30 (“NKp30”), and the antigen binding domain that binds to TRBC1 (“TRBC1”). In some embodiments, the antigen binding domain that bind NKp30 comprises an scFv. In some embodiments, the antigen binding domain that bind NKp30 comprises an Fab. In some embodiments, the antigen binding domain that binds to TRBC1 comprises an scFv. In some embodiments, the antigen binding domain that binds to TRBC1 comprises an Fab. FIG. 1A depicts an agent comprising an anti-TRBC1 Fab, an anti-NKp30 scFv, and an Fe dimer. The Fe dimer comprises two Fc chains. The C-terminus of the heavy chain of the anti-TRBC1 Fab is fused to the N-terminus of one Fc chain. The anti-NKp30 scFv is fused to the N-terminus of the other Fc chain. FIG. 1B depicts an agent comprising an anti-TRBC1 Fab, an anti-NKp30 Fab, and an Fe dimer. The Fe dimer comprises two Fc chains. The C-terminus of the heavy chain of the anti-TRBC1 Fab is fused to the N-terminus of one Fc chain. The C-terminus of the heavy chain of the anti-NKp30 Fab is fused to the N-terminus of the other Fc chain. FIG. 1C depicts an agent comprising an anti-TRBC1 scFv, an anti-NKp30 Fab and an Fe dimer. The Fe dimer comprises two Fc chains. The anti-TRBC1 scFv is fused to the N-terminus of the other Fc chain. The C-terminus of the heavy chain of the anti-NKp30 Fab is fused to the N-terminus of one Fc chain. FIG. 1D depicts an agent comprising an anti-NKp30 scFv, an anti-TRBC1 scFv, and an Fe dimer. The Fe dimer comprises two Fc chains. The anti-TRBC1 scFv is fused to the N-terminus of one Fc chain. The anti-NKp30 scFv is fused to the N-terminus of the other Fc chain. In some embodiments, the antigen binding domain that binds to NKp30 is linked to the C-terminus of the first member of the dimerization module or the C-terminus of the second member of the dimerization module. In some embodiments, the agent comprises more than one antigen binding domains that bind to NKp30, and the more than one antigen binding domains that bind to NKp30 are independently linked to the N-terminus of the first member of the dimerization module, the N-terminus of the second member of the dimerization module, the C-terminus of the first member of the dimerization module, the C-terminus of the second member of the dimerization module, or any combination thereof. In some embodiments, the antigen binding domain that binds to TRBC1 is linked to the C-terminus of the first member of the dimerization module or the C-terminus of the second member of the dimerization module. In some embodiments, the agent comprises more than one antigen binding domains that bind to TRBC1, and the more than one antigen binding domains that bind to TRBC1 are independently linked to the N-terminus of the first member of the dimerization module, the N-terminus of the second member of the dimerization module, the C-terminus of the first member of the dimerization module, the C-terminus of the second member of the dimerization module, or any combination thereof.

FIGS. 2A-2D are schematic representations of exemplary formats and configurations of the agents as provided herein. In some embodiments, the agent comprises the antigen binding domain that bind to NKp30 (“NKp30”), and the antigen binding domain that binds to TRBC2 (“TRBC2”). In some embodiments, the antigen binding domain that bind NKp30 comprises an scFv. In some embodiments, the antigen binding domain that bind NKp30 comprises an Fab. In some embodiments, the antigen binding domain that binds to TRBC2 comprises an scFv. In some embodiments, the antigen binding domain that binds to TRBC2 comprises an Fab. FIG. 2A depicts an agent comprising an anti-TRBC2 Fab, an anti-NKp30 scFv, and an Fe dimer. The Fe dimer comprises two Fc chains. The C-terminus of the heavy chain of the anti-TRBC2 Fab is fused to the N-terminus of one Fc chain. The anti-NKp30 scFv is fused to the N-terminus of the other Fc chain. FIG. 2B depicts an agent comprising an anti-TRBC2 Fab, an anti-NKp30 Fab, and an Fe dimer. The Fe dimer comprises two Fc chains. The C-terminus of the heavy chain of the anti-TRBC2 Fab is fused to the N-terminus of one Fc chain. The C-terminus of the heavy chain of the anti-NKp30 Fab is fused to the N-terminus of the other Fc chain. FIG. 2C depicts an agent comprising an anti-TRBC2 ScFv, an anti-NKp30 Fab and an Fe dimer. The Fe dimer comprises two Fc chains. The anti-TRBC2 scFv is fused to the N-terminus of the other Fc chain. The C-terminus of the heavy chain of the anti-NKp30 Fab is fused to the N-terminus of one Fc chain. FIG. 2D depicts an agent comprising an anti-NKp30 scFv, an anti-TRBC2 scFv, and an Fe dimer. The Fe dimer comprises two Fc chains. The anti-TRBC2 scFv is fused to the N-terminus of one Fc chain. The anti-NKp30 scFv is fused to the N-terminus of the other Fc chain. In some embodiments, the antigen binding domain that binds to NKp30 is linked to the C-terminus of the first member of the dimerization module or the C-terminus of the second member of the dimerization module. In some embodiments, the agent comprises more than one antigen binding domains that bind to NKp30, and the more than one antigen binding domains that bind to NKp30 are independently linked to the N-terminus of the first member of the dimerization module, the N-terminus of the second member of the dimerization module, the C-terminus of the first member of the dimerization module, the C-terminus of the second member of the dimerization module, or any combination thereof. In some embodiments, the antigen binding domain that binds to TRBC2 is linked to the C-terminus of the first member of the dimerization module or the C-terminus of the second member of the dimerization module. In some embodiments, the agent comprises more than one antigen binding domain that binds to TRBC2, and the more than one antigen binding domain that binds to TRBC2 are independently linked to the N-terminus of the first member of the dimerization module, the N-terminus of the second member of the dimerization module, the C-terminus of the first member of the dimerization module, the C-terminus of the second member of the dimerization module, or any combination thereof.

FIGS. 3A-3B are schematic representations of exemplary formats and configurations of the agents as provided herein. In some embodiments, the agent comprises the antigen binding domain that bind NKp30 (“Nkp30”) and at least one cytokine molecule or a functional fragment or variant thereof (“cytokine”). In some embodiments, the antigen binding domain that bind NKp30 comprises an scFv. In some embodiments, the antigen binding domain that bind NKp30 comprises an Fab. FIG. 3A depicts an agent comprising an anti-NKp30 scFv, a cytokine molecule, and an Fe dimer. The Fe dimer comprises two Fc chains. The anti-NKp30 scFv is fused to the N-terminus of one Fc chain. The cytokine molecule is fused to the N-terminus of the other Fc chain. FIG. 3B depicts an agent comprising an anti-NKp30 Fab, a cytokine molecule, and an Fe dimer. The Fe dimer comprises two Fc chains. The C-terminus of the heavy chain of the anti-NKp30 Fab is fused to the N-terminus of one Fc chain. The cytokine molecule is fused to the N-terminus of the other Fc chain. In some embodiments, the antigen binding domain that binds to NKp30 is linked to the C-terminus of the first member of the dimerization module or the C-terminus of the second member of the dimerization module. In some embodiments, the agent comprises more than one antigen binding domains that bind to NKp30, and the more than one antigen binding domains that bind to NKp30 are independently linked to the N-terminus of the first member of the dimerization module, the N-terminus of the second member of the dimerization module, the C-terminus of the first member of the dimerization module, the C-terminus of the second member of the dimerization module, or any combination thereof. In some embodiments, the at least one cytokine molecule or a functional fragment or variant thereof is linked to the C-terminus of the first member of the dimerization module or the C-terminus of the second member of the dimerization module. In some embodiments, the agent comprises more than one cytokine molecule or a functional fragment or variant thereof, and the more than one cytokine molecule or a functional fragment or variant thereof are independently linked to the N-terminus of the first member of the dimerization module, the N-terminus of the second member of the dimerization module, the C-terminus of the first member of the dimerization module, the C-terminus of the second member of the dimerization module, or any combination thereof.

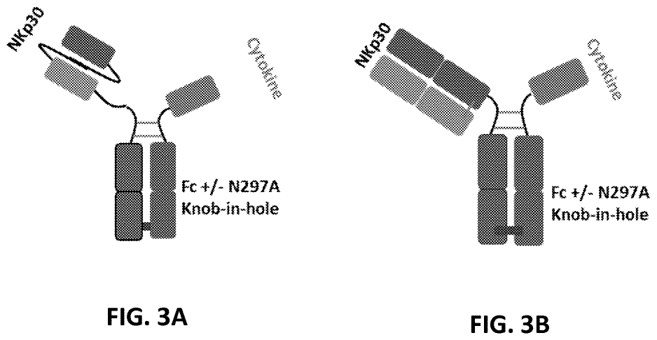

In some embodiments, the agent comprises the antigen binding domain that binds to NKp30, the antigen binding domain that binds to TRBC1, and the at least one cytokine molecule or a functional fragment or variant thereof. In some embodiments, the antigen binding domain that binds to NKp30, the antigen binding domain that binds to TRBC1, and the at least one cytokine molecule or a functional fragment or variant thereof are independently linked to the N-terminus of the first member of the dimerization module, the N-terminus of the second member of the dimerization module, the C-terminus of the first member of the dimerization module, the C-terminus of the second member of the dimerization module, or any combination thereof. In some embodiments, the agent comprises the antigen binding domain that binds to NKp30, the antigen binding domain that binds to TRBC2, and the at least one cytokine molecule or a functional fragment or variant thereof. In some embodiments, the antigen binding domain that binds to NKp30, the antigen binding domain that binds to TRBC2, and the at least one cytokine molecule or a functional fragment or variant thereof are independently linked to the N-terminus of the first member of the dimerization module, the N-terminus of the second member of the dimerization module, the C-terminus of the first member of the dimerization module, the C-terminus of the second member of the dimerization module, or any combination thereof. In some embodiments, the agent comprises the antigen binding domain that binds to NKp30, the antigen binding domain that binds to TRBC1 and/or the antigen binding domain that binds to TRBC2, and the at least one cytokine molecule or a functional fragment or variant thereof. In some embodiments, the antigen binding domain that binds to NKp30, the antigen binding domain that binds to TRBC1 and/or the antigen binding domain that binds to TRBC2, and the at least one cytokine molecule or a functional fragment or variant thereof are independently linked to the N-terminus of the first member of the dimerization module, the N-terminus of the second member of the dimerization module, the C-terminus of the first member of the dimerization module, the C-terminus of the second member of the dimerization module, or any combination thereof.

### DETAILED DESCRIPTION

### Definitions

In some embodiments, the multifunctional molecule includes an immune cell engager. “An immune cell engager” refers to one or more binding specificities that bind and/or activate an immune cell, e.g., a cell involved in an immune response. In embodiments, the immune cell is chosen from a T cell, an NK cell, a B cell, a dendritic cell, and/or the macrophage cell. The immune cell engager can be an antibody molecule, a receptor molecule (e.g., a full length receptor, receptor fragment, or fusion thereof (e.g., a receptor-Fc fusion)), or a ligand molecule (e.g., a full length ligand, ligand fragment, or fusion thereof (e.g., a ligand-Fc fusion)) that binds to the immune cell antigen (e.g., the T cell, the NK cell antigen, the B cell antigen, the dendritic cell antigen, and/or the macrophage cell antigen). In embodiments, the immune cell engager specifically binds to the target immune cell, e.g., binds preferentially to the target immune cell. For example, when the immune cell engager is an antibody molecule, it binds to an immune cell antigen (e.g., a T cell antigen, an NK cell antigen, a B cell antigen, a dendritic cell antigen, and/or a macrophage cell antigen) with a dissociation constant of less than about 10 nM.

In some embodiments, the multifunctional molecule includes a cytokine molecule. As used herein, a “cytokine molecule” refers to full length, a fragment or a variant of a cytokine; a cytokine further comprising a receptor domain, e.g., a cytokine receptor dimerizing domain; or an agonist of a cytokine receptor, e.g., an antibody molecule (e.g., an agonistic antibody) to a cytokine receptor, that elicits at least one activity of a naturally-occurring cytokine. In some embodiments the cytokine molecule is chosen from interleukin-2 (IL-2), interleukin-7 (IL-7), interleukin-12 (IL-12), interleukin-15 (IL-15), interleukin-18 (IL-18), interleukin-21 (IL-21), or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain. In other embodiments, the cytokine molecule is an agonist of a cytokine receptor, e.g., an antibody molecule (e.g., an agonistic antibody) to a cytokine receptor chosen from an IL-15Ra or IL-21R.

As used herein, the term “molecule” as used in, e.g., antibody molecule, cytokine molecule, receptor molecule, includes full-length, naturally-occurring molecules, as well as variants, e.g., functional variants (e.g., truncations, fragments, mutated (e.g., substantially similar sequences) or derivatized form thereof), so long as at least one function and/or activity of the unmodified (e.g., naturally-occurring) molecule remains.

In some embodiments, the multifunctional molecule includes a stromal modifying moiety. A “stromal modifying moiety,” as used herein refers to an agent, e.g., a protein (e.g., an enzyme), that is capable of altering, e.g., degrading a component of, the stroma. In embodiments, the component of the stroma is chosen from, e.g., an ECM component, e.g., a glycosaminoglycan, e.g., hyaluronan (also known as hyaluronic acid or HA), chondroitin sulfate, chondroitin, dermatan sulfate, heparin sulfate, heparin, entactin, tenascin, aggrecan and keratin sulfate; or an extracellular protein, e.g., collagen, laminin, elastin, fibrinogen, fibronectin, and vitronectin.

Certain terms are defined below.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values.

“Antibody molecule” as used herein refers to a protein, e.g., an immunoglobulin chain or fragment thereof, comprising at least one immunoglobulin variable domain sequence. An antibody molecule encompasses antibodies (e.g., full-length antibodies) and antibody fragments. In an embodiment, an antibody molecule comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab′, F(ab′)₂, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)₂ fragments, and single chain variable fragments (scFvs).

As used herein, an “immunoglobulin variable domain sequence” refers to an amino acid sequence which can form the structure of an immunoglobulin variable domain. For example, the sequence may include all or part of the amino acid sequence of a naturally occurring variable domain. For example, the sequence may or may not include one, two, or more N- or C-terminal amino acids, or may include other alterations that are compatible with formation of the protein structure.

In embodiments, an antibody molecule is monospecific, e.g., it comprises binding specificity for a single epitope. In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule is a bispecific antibody molecule. “Bispecific antibody molecule” as used herein refers to an antibody molecule that has specificity for more than one (e.g., two, three, four, or more) epitope and/or antigen.

“Antigen” (Ag) as used herein refers to a molecule that can provoke an immune response, e.g., involving activation of certain immune cells and/or antibody generation. Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full length nucleotide sequence of a gene, nor does an antigen need to be encoded by a gene at all. In embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response. As used, herein an “immune cell antigen” includes any molecule present on, or associated with, an immune cell that can provoke an immune response.

The “antigen-binding site,” or “binding portion” of an antibody molecule refers to the part of an antibody molecule, e.g., an immunoglobulin (Ig) molecule, that participates in antigen binding. In embodiments, the antigen binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions,” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, or by ImMunoGeneTics (IMGT) numbering system. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4.

As used herein, the term “NKp30”, also known as Natural cytotoxicity triggering receptor 3, CD337 (cluster of differentiation 337), NCR3, 1C7, LY117, MALS, natural cytotoxicity triggering receptor 3, NCR3, nkp30, or NKP30, as used interchangeably herein, refers to the type I transmembrane receptor belonging to the immunoglobulin (Ig) superfamily that is expressed on resting and active natural killer (NK) cells. In some embodiments, NKp30 can trigger cytotoxic response against tumor cells. The term “NKp30” includes isoforms, mammalian e.g., human NKp30, species homologs of human and analogs comprising at least one common epitope with NKp30. Human NKp30 comprises isoforms including, but not limited to: NKp30A, NKp30B, or NKp30C. Also, the term “NKp30” includes any of the recombinant or naturally-occurring forms of NKp30 or variants or homologs thereof that have or maintain NKp30 activity (e.g., at least 40% 50%, 60%, 70%, 80%, 90%, 95%, 96%, 97%, 98%, 99% or 100% activity). In some aspects, the variants or homologs have at least 40%, 45%, 50%, 55%, 60%, 65%, 70%, 75%, 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring NKp30. In some embodiments, NKp30 is substantially identical to the protein identified by the UniProt reference number 014931 or a variant or homolog having substantial identity thereto.

As used herein, the terms “T cell receptor beta variable chain,” “T cell receptor beta chain variable region” “TCRβV,” “TCRβ V,” “TCR βV,” “TCRβv,” “TCR βv,” “TCRβ v,” “T cell receptor variable beta chain,” “TCRβV,” “TCR Vβ,” “TCRV β,” “TCRβV,” “TCRv β,” “TCR vβ,” “TCRβV,” “TCRVB,” “TRBV,” or “TCR beta V” are used interchangeably herein and refer to an extracellular region of the T cell receptor beta chain which comprises the antigen recognition domain of the T cell receptor. The term TCRβV includes isoforms, mammalian, e.g., human TCRβV, species homologs of human and analogs comprising at least one common epitope with TCRβV. Human TCRβV comprises a gene family comprising subfamilies including, but not limited to: a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily. In some embodiments, the TCRβ V6 subfamily comprises: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, TCRβV comprises TCRβ V6-5*01. TCRβ V6-5*01 is also known as TRBV65; TCRβV 6S5; TCRβV 13S1, or TCRβV 13.1. The amino acid sequence of TCRβ V6-5*01, e.g., human TCRβ V6-5*01, is known in that art, e.g., as provided by IMGT ID L36092.

“Cancer” as used herein can encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer. The terms “cancer” and “tumor” can be used interchangeably. For example, both terms encompass solid and liquid tumors. As used herein, the term “cancer” or “tumor” includes premalignant, as well as malignant cancers and tumors.

As used herein, an “immune cell” refers to any of various cells that function in the immune system, e.g., to protect against agents of infection and foreign matter. In embodiments, this term includes leukocytes, e.g., neutrophils, eosinophils, basophils, lymphocytes, and monocytes. Innate leukocytes include phagocytes (e.g., macrophages, neutrophils, and dendritic cells), mast cells, eosinophils, basophils, and natural killer cells. Innate leukocytes identify and eliminate pathogens, either by attacking larger pathogens through contact or by engulfing and then killing microorganisms, and are mediators in the activation of an adaptive immune response. The cells of the adaptive immune system are special types of leukocytes, called lymphocytes. B cells and T cells are important types of lymphocytes and are derived from hematopoietic stem cells in the bone marrow. B cells are involved in the humoral immune response, whereas T cells are involved in cell-mediated immune response. The term “immune cell” includes immune effector cells.

“Immune effector cell,” as that term is used herein, refers to a cell that is involved in an immune response, e.g., in the promotion of an immune effector response. Examples of immune effector cells include, but are not limited to, T cells, e.g., alpha/beta T cells and gamma/delta T cells, B cells, natural killer (NK) cells, natural killer T (NK T) cells, and mast cells.

The term “effector function” or “effector response” refers to a specialized function of a cell. Effector function of a T cell, for example, may be cytolytic activity or helper activity including the secretion of cytokines.

The compositions and methods of the present invention encompass polypeptides and nucleic acids having the sequences specified, or sequences substantially identical or similar thereto, e.g., sequences at least 80%, 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 80%, 85%, 90%, 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 80%, 85%, 90%, 91%, 92%, 93%, 94%, 95%, 96%, 97%, 98% or 99% identity to a reference sequence, e.g., a sequence provided herein.

The term “variant” refers to a polypeptide that has a substantially identical amino acid sequence to a reference amino acid sequence, or is encoded by a substantially identical nucleotide sequence. In some embodiments, the variant is a functional variant.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

The terms “complementarity determining region” or “CDR,” are used interchangeably herein and refer to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme), ImMunoGeneTics (IMGT) numbering system, or any combination thereof. Under the Kabat numbering scheme, in some embodiments, the CDR amino acid residues in the heavy chain variable domain (V_{H}) are numbered 31-35 (HCDR1), 50-65 (HCDR2), and 95-102 (HCDR3); and the CDR amino acid residues in the light chain variable domain (V_{L}) are numbered 24-34 (LCDR1), 50-56 (LCDR2), and 89-97 (LCDR3). Under the Chothia numbering scheme, in some embodiments, the CDR amino acids in the V_{H} are numbered 26-32 (HCDR1), 52-56 (HCDR2), and 95-102 (HCDR3); and the CDR amino acid residues in the V_{L} are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a V_{H}, e.g., a mammalian V_{H}, e.g., a human V_{H}; and amino acid residues 24-34 (LCDR1), 50-56 (LCDR2), and 89-97 (LCDR3) in a V_{L}, e.g., a mammalian V_{L}, e.g., a human V_{L}.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody or antibody fragment, where the whole molecule is of human origin or consists of an amino acid sequence identical to a human form of the antibody or immunoglobulin.

The term “specifically binds,” refers to an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

As used herein, an “immune cell” refers to any of various cells that function in the immune system, e.g., to protect against agents of infection and foreign matter. In embodiments, this term includes leukocytes, e.g., neutrophils, eosinophils, basophils, lymphocytes, and monocytes. Innate leukocytes include phagocytes (e.g., macrophages, neutrophils, and dendritic cells), mast cells, eosinophils, basophils, and natural killer cells. Innate leukocytes identify and eliminate pathogens, either by attacking larger pathogens through contact or by engulfing and then killing microorganisms, and are mediators in the activation of an adaptive immune response. The cells of the adaptive immune system are special types of leukocytes, called lymphocytes. B cells and T cells are important types of lymphocytes and are derived from hematopoietic stem cells in the bone marrow. B cells are involved in the humoral immune response, whereas T cells are involved in cell-mediated immune response. The term “immune cell” includes immune effector cells.

As used herein the term “immune effector cell,” refers to a cell that is involved in an immune response, e.g., in the promotion of an immune effector response. Examples of immune effector cells include, but are not limited to, T cells (e.g., alpha/beta T cells, gamma/delta T cells CD4+ T cells, CD8+ T cells), B cells, natural killer (NK) cells, natural killer T (NK T) cells, monocytes, macrophages, neutrophils, basophils, dendritic cells and mast cells.

The terms “effector function” or “effector response” refer to a specialized function of a cell. Effector function of a T cell, for example, may be cytolytic activity (e.g., CD8+ T cells) or helper activity (e.g., CD4+ T cells) including the secretion of cytokines.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

The term, a “substantially purified cell” or “substantially purified cell population” refers to a cell or cell population that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

“Derived from” as that term is used herein, indicates a relationship between a first and a second molecule. It generally refers to structural similarity between the first molecule and a second molecule and does not connote or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connote or include a limitation to a particular process of producing the intracellular signaling domain, e.g., it does not mean that, to provide the intracellular signaling domain, one must start with a CD3zeta sequence and delete unwanted sequence, or impose mutations, to arrive at the intracellular signaling domain.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecule of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See ncbi.nlm.nih.gov.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally occurring amino acids. Exemplary amino acids include naturally occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Such conservative modifications include amino acid substitutions, additions and deletions. Modifications can be introduced into an antibody or antibody fragment by standard techniques known in the art, such as site-directed mutagenesis and PCR-mediated mutagenesis. Conservative substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR can be replaced with other amino acid residues from the same side chain family and the altered CAR can be tested using the functional assays described herein

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “vector” as used herein refers to any vehicle that can be used to deliver and/or express a nucleic acid molecule. It can be a transfer vector or an expression vector as described herein.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive promoter” refers to a nucleotide sequence which, when operably linked with a polynucleotide which encodes or specifies a gene product, causes the gene product to be produced in a cell under most or all physiological conditions of the cell.

The term “inducible promoter” refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific promoter” refers to a nucleotide sequence which, when operably linked with a polynucleotide encodes or specified by a gene, causes the gene product to be produced in a cell substantially only if the cell is a cell of the tissue type corresponding to the promoter.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “chimeric antigen receptor” or alternatively a “CAR” are used interchangeably herein and refer to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains. In one aspect, the stimulatory molecule of the CAR is the zeta chain associated with the T cell receptor complex. In one aspect, the cytoplasmic signaling domain comprises a primary signaling domain (e.g., a primary signaling domain of CD3-zeta). In one aspect, the cytoplasmic signaling domain further comprises one or more functional signaling domains derived from at least one costimulatory molecule as defined below. In one aspect, the costimulatory molecule is chosen from 4-1BB (i.e., CD137), CD27, ICOS, and/or CD28. In one aspect, the CAR comprises a chimeric fusion protein comprising an extracellular antigen recognition domain, a transmembrane domain and an intracellular signaling domain comprising a functional signaling domain derived from a stimulatory molecule. In one aspect, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In one aspect, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. In one aspect, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the antigen recognition domain (e.g., a scFv) during cellular processing and localization of the CAR to the cellular membrane.

The term “signaling domain” as used herein refers to the functional portion of a protein which acts by transmitting information within the cell to regulate cellular activity via defined signaling pathways by generating second messengers or functioning as effectors by responding to such messengers.

An “intracellular signaling domain,” as the term is used herein, refers to an intracellular portion of a molecule. The intracellular signaling domain can generate a signal that promotes an immune effector function of the CAR containing cell, e.g., a CART cell or CAR-expressing NK cell. Examples of immune effector function, e.g., in a CART cell or CAR-expressing NK cell, include cytolytic activity and helper activity, including the secretion of cytokines. In embodiments, the intracellular signal domain transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal. In some embodiment, the intracellular signaling domain comprises a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CAR-expressing immune effector cell, e.g., CART cell or CAR-expressing NK cell, a primary intracellular signaling domain can comprise a cytoplasmic sequence of a T cell receptor, and a costimulatory intracellular signaling domain can comprise cytoplasmic sequence from co-receptor or costimulatory molecule. A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CDS, CD22, CD79a, CD79b, CD278 (“ICOS”), FcεRI, CD66d, DAP10, and DAP12.

The term “zeta” or alternatively “zeta chain”, “CD3-zeta” or “TCR-zeta” is defined as the protein provided as GenBan Acc. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain that are sufficient to functionally transmit an initial signal necessary for T cell activation. In one aspect the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an a MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, V_{L}A1, CD49a, ITGA4, IA4, CD49D, ITGA6, V_{L}A-6, CD49f, ITGAD, CD1 Id, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Ly108), SLAM (SLAMFI, CD150, IPO-3), BLAME (SLAMF8), SELPLG (CD162), LTBR, LAT, GADS, SLP-76, PAG/Cbp, CD19a, and a ligand that specifically binds with CD83.

A “costimulatory intracellular signaling domain” refers to the intracellular portion of a costimulatory molecule. The intracellular signaling domain can comprise the entire intracellular portion, or the entire native intracellular signaling domain, of the molecule from which it is derived, or a functional fragment thereof.

The term “signal transduction pathway” as used herein refers to the biochemical relationship between a variety of signal transduction molecules that play a role in the transmission of a signal from one portion of a cell to another portion of a cell.

The term “cell surface receptor” as used herein includes molecules and complexes of molecules capable of receiving a signal and transmitting signal across the membrane of a cell.

The term “anti-tumor effect” or “anti-cancer effect,” used interchangeably herein refer to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies in prevention of the occurrence of tumor in the first place.

The terms “Cancer” or “tumor” as used interchangeably herein and encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer. For example, both terms encompass solid and liquid tumors. As used herein, the term cancer includes premalignant, as well as malignant cancers and tumors.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species.

The term “apheresis” as used herein refers to the art-recognized extracorporeal process by which the blood of a donor or patient is removed from the donor or patient and passed through an apparatus that separates out selected particular constituent(s) and returns the remainder to the circulation of the donor or patient, e.g., by re-transfusion. Thus, in the context of “an apheresis sample” refers to a sample obtained using apheresis.

The term “combination” refers to either a fixed combination in one dosage unit form, or a combined administration where a compound and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound and a combination partner, are both administered to a patient simultaneously in the form of a single entity or dosage. The term “non-fixed combination” means that the active ingredients, e.g. a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

The term “effective amount” or “therapeutically effective amount” are used interchangeably herein, and refer to an amount of a compound, formulation, material, or composition, as described herein effective to achieve a particular biological result.

As used herein, the terms “treat,” “treatment,” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (e.g., one or more therapeutic agents such as a CAR). In specific embodiments, the terms “treat,” “treatment,” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, e.g., stabilization of a discernible symptom, physiologically by, e.g., stabilization of a physical parameter, or both. In other embodiments the terms “treat,” “treatment,” and “treating” refer to the reduction or stabilization of tumor size or cancerous cell count.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

The term “subject” is intended to include living organisms in which an immune response can be elicited (e.g., mammals, human).

Various aspects of the invention are described in further detail below. Additional definitions are set out throughout the specification.

### Antibody Molecules

In one embodiment, the antibody molecule binds to a cancer antigen, e.g., a tumor antigen or a stromal antigen. In some embodiments, the cancer antigen is, e.g., a mammalian, e.g., a human, cancer antigen. In other embodiments, the antibody molecule binds to an immune cell antigen, e.g., a mammalian, e.g., a human, immune cell antigen. For example, the antibody molecule binds specifically to an epitope, e.g., linear or conformational epitope, on the cancer antigen or the immune cell antigen.

In an embodiment, an antibody molecule is a monospecific antibody molecule and binds a single epitope. E.g., a monospecific antibody molecule having a plurality of immunoglobulin variable domain sequences, each of which binds the same epitope.

In an embodiment an antibody molecule is a multispecific or multifunctional antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a multispecific antibody molecule comprises a third, fourth or fifth immunoglobulin variable domain. In an embodiment, a multispecific antibody molecule is a bispecific antibody molecule, a trispecific antibody molecule, or a tetraspecific antibody molecule.

In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv or a Fab, or fragment thereof, have binding specificity for a first epitope and a scFv or a Fab, or fragment thereof, have binding specificity for a second epitope.

In an embodiment, an antibody molecule comprises a diabody, and a single-chain molecule, as well as an antigen-binding fragment of an antibody (e.g., Fab, F(ab′)₂, and Fv). For example, an antibody molecule can include a heavy (H) chain variable domain sequence (abbreviated herein as VH), and a light (L) chain variable domain sequence (abbreviated herein as VL). In an embodiment an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. A preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

Antibody molecules include intact molecules as well as functional fragments thereof. Constant regions of the antibody molecules can be altered, e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of known schemes, including those described by Kabat et al. (1991), “Sequences of Proteins of Immunological Interest,” 5^{th} Ed. Public Health Service, National Institutes of Health, Bethesda, MD (“Kabat” numbering scheme), Al-Lazikani et al., (1997) JMB 273,927-948 (“Chothia” numbering scheme), or by ImMunoGeneTics (IMGT) numbering system. As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3).

Each VH and VL typically includes three CDRs and four FRs, arranged from amino-terminus to carboxy-terminus in the following order: FR1, CDR1, FR2, CDR2, FR3, CDR3, FR4.

The antibody molecule can be a polyclonal or a monoclonal antibody.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

The antibody can be recombinantly produced, e.g., produced by phage display or by combinatorial methods.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody molecule can be one in which the variable region, or a portion thereof, e.g., the CDRs, are generated in a non-human organism, e.g., a rat or mouse. Chimeric, CDR-grafted, and humanized antibodies are within the invention. Antibody molecules generated in a non-human organism, e.g., a rat or mouse, and then modified, e.g., in the variable framework or constant region, to decrease antigenicity in a human are within the invention.

An “effectively human” protein is a protein that does substantially not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding to the antigen. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody molecule can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibody molecules can be produced by CDR-grafting or CDR substitution, wherein one, two, or all CDRs of an immunoglobulin chain can be replaced. See, e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibody molecules in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFv) may be engineered (see, for example, Colcher, D. et al. (1999) Ann NY Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

### Multispecific or Multifunctional Antibody Molecules

As used herein, a “multifunctional” or a “multispecific” molecule refers to molecule, e.g., a polypeptide, that has two or more functionalities, e.g., two or more binding specificities. In some embodiments, the functionalities can include one or more immune cell engagers, one or more tumor binding molecules, one or more cytokine molecules, one or more stromal modifiers, and other moieties described herein. In some embodiments, the multispecific molecule is a multispecific antibody molecule, e.g., a bispecific antibody molecule.

Exemplary structures of multispecific and multifunctional molecules defined herein are described throughout. Exemplary structures are further described in: Weidle U et al. (2013) The Intriguing Options of Multispecific Antibody Formats for Treatment of Cancer. Cancer Genomics & Proteomics 10: 1-18 (2013); and Spiess C et al. (2015) Alternative molecular formats and therapeutic applications for bispecific antibodies. Molecular Immunology 67: 95-106; the full contents of each of which is incorporated by reference herein).

In embodiments, multispecific antibody molecules can comprise more than one antigen-binding site, where different sites are specific for different antigens. In embodiments, multispecific antibody molecules can bind more than one (e.g., two or more) epitopes on the same antigen. In embodiments, multispecific antibody molecules comprise an antigen-binding site specific for a target cell (e.g., cancer cell) and a different antigen-binding site specific for an immune effector cell. In one embodiment, the multispecific antibody molecule is a bispecific antibody molecule. Bispecific antibody molecules can be classified into five different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates.

BsIgG is a format that is monovalent for each antigen. Exemplary BsIgG formats include but are not limited to crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, κλ-body, orthogonal Fab. See Spiess et al. Mol. Immunol. 67(2015):95-106. Exemplary BsIgGs include catumaxomab (Fresenius Biotech, Trion Pharma, Neopharm), which contains an anti-CD3 arm and an anti-EpCAM arm; and ertumaxomab (Neovii Biotech, Fresenius Biotech), which targets CD3 and HER2. In some embodiments, BsIgG comprises heavy chains that are engineered for heterodimerization. For example, heavy chains can be engineered for heterodimerization using a “knobs-into-holes” strategy, a SEED platform, a common heavy chain (e.g., in κλ-bodies), and use of heterodimeric Fe regions. See Spiess et al. Mol. Immunol. 67(2015):95-106. Strategies that have been used to avoid heavy chain pairing of homodimers in BsIgG include knobs-in-holes, duobody, azymetric, charge pair, HA-TF, SEEDbody, and differential protein A affinity. See Id. BsIgG can be produced by separate expression of the component antibodies in different host cells and subsequent purification/assembly into a BsIgG. BsIgG can also be produced by expression of the component antibodies in a single host cell. BsIgG can be purified using affinity chromatography, e.g., using protein A and sequential pH elution.

IgG appended with an additional antigen-binding moiety is another format of bispecific antibody molecules. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). See Id. Examples of appended IgG formats include dual variable domain IgG (DVD-Ig), IgG(H)-scFv, scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67(2015):95-106. An example of an IgG-scFv is MM-141 (Merrimack Pharmaceuticals), which binds IGF-1R and HER3. Examples of DVD-Ig include ABT-981 (AbbVie), which binds IL-1α and IL-1β; and ABT-122 (AbbVie), which binds TNF and IL-17A.

Bispecific antibody fragments (BsAb) are a format of bispecific antibody molecules that lack some or all of the antibody constant domains. For example, some BsAb lack an Fc region. In embodiments, bispecific antibody fragments include heavy and light chain regions that are connected by a peptide linker that permits efficient expression of the BsAb in a single host cell. Exemplary bispecific antibody fragments include but are not limited to nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fe, Diabody-Fe, tandem scFv-Fc, and intrabody. See Id. For example, the BiTE format comprises tandem scFvs, where the component scFvs bind to CD3 on T cells and a surface antigen on cancer cells

Bispecific fusion proteins include antibody fragments linked to other proteins, e.g., to add additional specificity and/or functionality. An example of a bispecific fusion protein is an immTAC, which comprises an anti-CD3 scFv linked to an affinity-matured T-cell receptor that recognizes HLA-presented peptides. In embodiments, the dock-and-lock (DNL) method can be used to generate bispecific antibody molecules with higher valency. Also, fusions to albumin binding proteins or human serum albumin can be extend the serum half-life of antibody fragments. See Id.

In embodiments, chemical conjugation, e.g., chemical conjugation of antibodies and/or antibody fragments, can be used to create BsAb molecules. See Id. An exemplary bispecific antibody conjugate includes the CovX-body format, in which a low molecular weight drug is conjugated site-specifically to a single reactive lysine in each Fab arm or an antibody or fragment thereof. In embodiments, the conjugation improves the serum half-life of the low molecular weight drug. An exemplary CovX-body is CVX-241 (NCT01004822), which comprises an antibody conjugated to two short peptides inhibiting either VEGF or Ang2. See Id.

The antibody molecules can be produced by recombinant expression, e.g., of at least one or more component, in a host system. Exemplary host systems include eukaryotic cells (e.g., mammalian cells, e.g., CHO cells, or insect cells, e.g., SF9 or S2 cells) and prokaryotic cells (e.g., E. coli). Bispecific antibody molecules can be produced by separate expression of the components in different host cells and subsequent purification/assembly. Alternatively, the antibody molecules can be produced by expression of the components in a single host cell. Purification of bispecific antibody molecules can be performed by various methods such as affinity chromatography, e.g., using protein A and sequential pH elution. In other embodiments, affinity tags can be used for purification, e.g., histidine-containing tag, myc tag, or streptavidin tag.

### CDR-Grafted Scaffolds

In embodiments, the antibody molecule is a CDR-grafted scaffold domain. In embodiments, the scaffold domain is based on a fibronectin domain, e.g., fibronectin type III domain. The overall fold of the fibronectin type III (Fn3) domain is closely related to that of the smallest functional antibody fragment, the variable domain of the antibody heavy chain. There are three loops at the end of Fn3; the positions of BC, DE and FG loops approximately correspond to those of CDR1, 2 and 3 of the VH domain of an antibody. Fn3 does not have disulfide bonds; and therefore Fn3 is stable under reducing conditions, unlike antibodies and their fragments (see, e.g., WO 98/56915; WO 01/64942; WO 00/34784). An Fn3 domain can be modified (e.g., using CDRs or hypervariable loops described herein) or varied, e.g., to select domains that bind to an antigen/marker/cell described herein.

In embodiments, a scaffold domain, e.g., a folded domain, is based on an antibody, e.g., a “minibody” scaffold created by deleting three beta strands from a heavy chain variable domain of a monoclonal antibody (see, e.g., Tramontano et al., 1994, J Mol. Recognit. 7:9; and Martin et al., 1994, EMBO J. 13:5303-5309). The “minibody” can be used to present two hypervariable loops. In embodiments, the scaffold domain is a V-like domain (see, e.g., Coia et al. WO 99/45110) or a domain derived from tendamistatin, which is a 74 residue, six-strand beta sheet sandwich held together by two disulfide bonds (see, e.g., McConnell and Hoess, 1995, J Mol. Biol. 250:460). For example, the loops of tendamistatin can be modified (e.g., using CDRs or hypervariable loops) or varied, e.g., to select domains that bind to a marker/antigen/cell described herein. Another exemplary scaffold domain is a beta-sandwich structure derived from the extracellular domain of CTLA-4 (see, e.g., WO 00/60070).

Other exemplary scaffold domains include but are not limited to T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains). See, e.g., US 20040009530 and U.S. Pat. No. 7,501,121, incorporated herein by reference.

In embodiments, a scaffold domain is evaluated and chosen, e.g., by one or more of the following criteria: (1) amino acid sequence, (2) sequences of several homologous domains, (3) 3-dimensional structure, and/or (4) stability data over a range of pH, temperature, salinity, organic solvent, oxidant concentration. In embodiments, the scaffold domain is a small, stable protein domain, e.g., a protein of less than 100, 70, 50, 40 or 30 amino acids. The domain may include one or more disulfide bonds or may chelate a metal, e.g., zinc.

### Antibody-Based Fusions

A variety of formats can be generated which contain additional binding entities attached to the N or C terminus of antibodies. These fusions with single chain or disulfide stabilized Fvs or Fabs result in the generation of tetravalent molecules with bivalent binding specificity for each antigen. Combinations of scFvs and scFabs with IgGs enable the production of molecules which can recognize three or more different antigens.

### Antibody-Fab Fusion

Antibody-Fab fusions are bispecific antibodies comprising a traditional antibody to a first target and a Fab to a second target fused to the C terminus of the antibody heavy chain. Commonly the antibody and the Fab will have a common light chain. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Antibody-scFv Fusion

Antibody-scFv Fusions are bispecific antibodies comprising a traditional antibody and a scFv of unique specificity fused to the C terminus of the antibody heavy chain. The scFv can be fused to the C terminus through the Heavy Chain of the scFv either directly or through a linker peptide. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Variable Domain Immunoglobulin DVD

A related format is the dual variable domain immunoglobulin (DVD), which are composed of VH and VL domains of a second specificity place upon the N termini of the V domains by shorter linker sequences.

Other exemplary multispecific antibody formats include, e.g., those described in the following US20160114057A1, US20130243775A1, US20140051833, US20130022601, US20150017187A1, US20120201746A1, US20150133638A1, US20130266568A1, US20160145340A1, WO2015127158A1, US20150203591A1, US20140322221A1, US20130303396A1, US20110293613, US20130017200A1, US20160102135A1, WO2015197598A2, WO2015197582A1, U.S. Pat. No. 9,359,437, US20150018529, WO2016115274A1, WO2016087416A1, US20080069820A1, U.S. Pat. Nos. 9,145,588B, 7,919,257, and US20150232560A1. Exemplary multispecific molecules utilizing a full antibody-Fab/scFab format include those described in the following, U.S. Pat. No. 9,382,323B2, US20140072581A1, US20140308285A1, US20130165638A1, US20130267686A1, US20140377269A1, U.S. Pat. No. 7,741,446B2, and WO1995009917A1. Exemplary multispecific molecules utilizing a domain exchange format include those described in the following, US20150315296A1, WO2016087650A1, US20160075785A1, WO2016016299A1, US20160130347A1, US20150166670, U.S. Pat. No. 8,703,132B2, US20100316645, U.S. Pat. No. 8,227,577B2, US20130078249.

### Fc-Containing Entities (Mini-Antibodies)

Fc-containing entities, also known as mini-antibodies, can be generated by fusing scFv to the C-termini of constant heavy region domain 3 (CH3-scFv) and/or to the hinge region (scFv-hinge-Fc) of an antibody with a different specificity. Trivalent entities can also be made which have disulfide stabilized variable domains (without peptide linker) fused to the C-terminus of CH3 domains of IgGs.

### Fc-Containing Multispecific Molecules

In Some Embodiments, the Multispecific Molecules Disclosed Herein Includes an Immunoglobulin constant region (e.g., an Fc region). Exemplary Fc regions can be chosen from the heavy chain constant regions of IgG1, IgG2, IgG3 or IgG4; more particularly, the heavy chain constant region of human IgG1, IgG2, IgG3, or IgG4.

In some embodiments, the immunoglobulin chain constant region (e.g., the Fc region) is altered, e.g., mutated, to increase or decrease one or more of: Fc receptor binding, antibody glycosylation, the number of cysteine residues, effector cell function, or complement function.

In other embodiments, an interface of a first and second immunoglobulin chain constant regions (e.g., a first and a second Fc region) is altered, e.g., mutated, to increase or decrease dimerization, e.g., relative to a non-engineered interface, e.g., a naturally occurring interface. For example, dimerization of the immunoglobulin chain constant region (e.g., the Fc region) can be enhanced by providing an Fe interface of a first and a second Fc region with one or more of: a paired protuberance-cavity (“knob-in-a hole”), an electrostatic interaction, or a strand-exchange, such that a greater ratio of heteromultimer to homomultimer forms, e.g., relative to a non-engineered interface.

In some embodiments, the multispecific molecules include a paired amino acid substitution at a position chosen from one or more of 347, 349, 350, 351, 366, 368, 370, 392, 394, 395, 397, 398, 399, 405, 407, or 409, e.g., of the Fc region of human IgG1 For example, the immunoglobulin chain constant region (e.g., Fc region) can include a paired an amino acid substitution chosen from: T366S, L368A, or Y407V (e.g., corresponding to a cavity or hole), and T366W (e.g., corresponding to a protuberance or knob).

In other embodiments, the multifunctional molecule includes a half-life extender, e.g., a human serum albumin or an antibody molecule to human serum albumin.

### Heterodimerized Antibody Molecules & Methods of Making

Various methods of producing multispecific antibodies have been disclosed to address the problem of incorrect heavy chain pairing. Exemplary methods are described below. Exemplary multispecific antibody formats and methods of making said multispecific antibodies are also disclosed in e.g., Speiss et al. Molecular Immunology 67 (2015) 95-106; and Klein et al mAbs 4:6, 653-663; November/December 2012; the entire contents of each of which are incorporated by reference herein.

Heterodimerized bispecific antibodies are based on the natural IgG structure, wherein the two binding arms recognize different antigens. IgG derived formats that enable defined monovalent (and simultaneous) antigen binding are generated by forced heavy chain heterodimerization, combined with technologies that minimize light chain mispairing (e.g., common light chain). Forced heavy chain heterodimerization can be obtained using, e.g., knob-in-hole OR strand exchange engineered domains (SEED).

### Knob-in-Hole

Knob-in-Hole as described in U.S. Pat. Nos. 5,731,116, 7,476,724 and Ridgway, J. et al. (1996) Prot. Engineering 9(7): 617-621, broadly involves: (1) mutating the CH3 domain of one or both antibodies to promote heterodimerization; and (2) combining the mutated antibodies under conditions that promote heterodimerization. “Knobs” or “protuberances” are typically created by replacing a small amino acid in a parental antibody with a larger amino acid (e.g., T366Y or T366W); “Holes” or “cavities” are created by replacing a larger residue in a parental antibody with a smaller amino acid (e.g., Y407T, T366S, L368A and/or Y407V).

For bispecific antibodies including an Fc domain, introduction of specific mutations into the constant region of the heavy chains to promote the correct heterodimerization of the Fc portion can be utilized. Several such techniques are reviewed in Klein et al. (mAbs (2012) 4:6, 1-11), the contents of which are incorporated herein by reference in their entirety. These techniques include the “knobs-into-holes” (KiH) approach which involves the introduction of a bulky residue into one of the CH3 domains of one of the antibody heavy chains. This bulky residue fits into a complementary “hole” in the other CH3 domain of the paired heavy chain so as to promote correct pairing of heavy chains (see e.g., U.S. Pat. No. 7,642,228).

Exemplary KiH mutations include S354C, T366W in the “knob” heavy chain and Y349C, T366S, L368A, Y407V in the “hole” heavy chain. Other exemplary KiH mutations are provided in Table 1, with additional optional stabilizing Fc cysteine mutations.

Other Fe mutations are provided by Igawa and Tsunoda who identified 3 negatively charged residues in the CH3 domain of one chain that pair with three positively charged residues in the CH3 domain of the other chain. These specific charged residue pairs are: E356-K439, E357-K370, D399-K409 and vice versa. By introducing at least two of the following three mutations in chain A: E356K, E357K and D399K, as well as K370E, K409D, K439E in chain B, alone or in combination with newly identified disulfide bridges, they were able to favor very efficient heterodimerization while suppressing homodimerization at the same time (Martens T et al. A novel one-armed antic-Met antibody inhibits glioblastoma growth in vivo. Clin Cancer Res 2006; 12:6144-52; PMID:17062691). Xencor defined 41 variant pairs based on combining structural calculations and sequence information that were subsequently screened for maximal heterodimerization, defining the combination of S364H, F405A (HA) on chain A and Y349T, T394F on chain B (TF) (Moore G L et al. A novel bispecific antibody format enables simultaneous bivalent and monovalent co-engagement of distinct target antigens. MAbs 2011; 3:546-57; PMID: 22123055).

Other exemplary Fe mutations to promote heterodimerization of multispecific antibodies include those described in the following references, the contents of each of which is incorporated by reference herein, WO2016071377A1, US20140079689A1, US20160194389A1, US20160257763, WO2016071376A2, WO2015107026A1, WO2015107025A1, WO2015107015A1, US20150353636A1, US20140199294A1, U.S. Pat. No. 7,750,128B2, US20160229915A1, US20150344570A1, U.S. Pat. No. 8,003,774A1, US20150337049A1, US20150175707A1, US20140242075A1, US20130195849A1, US20120149876A1, US20140200331A1, U.S. Pat. No. 9,309,311B2, U.S. Pat. No. 8,586,713, US20140037621A1, US20130178605A1, US20140363426A1, US20140051835A1 and US20110054151A1.

Stabilizing cysteine mutations have also been used in combination with KiH and other Fe heterodimerization promoting variants, see e.g., U.S. Pat. No. 7,183,076. Other exemplary cysteine modifications include, e.g., those disclosed in US20140348839A1, U.S. Pat. Nos. 7,855,275B2, and 9,000,130B2.

### Strand Exchange Engineered Domains (SEED)

Heterodimeric Fc platform that support the design of bispecific and asymmetric fusion proteins by devising strand-exchange engineered domain (SEED) C(H)3 heterodimers are known. These derivatives of human IgG and IgA C(H)3 domains create complementary human SEED C(H)3 heterodimers that are composed of alternating segments of human IgA and IgG C(H)3 sequences. The resulting pair of SEED C(H)3 domains preferentially associates to form heterodimers when expressed in mammalian cells. SEEDbody (Sb) fusion proteins consist of [IgG1 hinge]-C(H)2-[SEED C(H)3], that may be genetically linked to one or more fusion partners (see e.g., Davis J H et al. SEEDbodies: fusion proteins based on strand exchange engineered domain (SEED) CH3 heterodimers in an Fe analogue platform for asymmetric binders or immunofusions and bispecific antibodies. Protein Eng Des Sel 2010; 23:195-202; PMID:20299542 and U.S. Pat. No. 8,871,912. The contents of each of which are incorporated by reference herein).

### Duobody

“Duobody” technology to produce bispecific antibodies with correct heavy chain pairing are known. The DuoBody technology involves three basic steps to generate stable bispecific human IgGlantibodies in a post-production exchange reaction. In a first step, two IgG1s, each containing single matched mutations in the third constant (CH3) domain, are produced separately using standard mammalian recombinant cell lines. Subsequently, these IgG1 antibodies are purified according to standard processes for recovery and purification. After production and purification (post-production), the two antibodies are recombined under tailored laboratory conditions resulting in a bispecific antibody product with a very high yield (typically >95%) (see e.g., Labrijn et al, PNAS 2013; 110(13):5145-5150 and Labrijn et al. Nature Protocols 2014; 9(10):2450-63, the contents of each of which are incorporated by reference herein).

### Electrostatic Interactions

Methods of making multispecific antibodies using CH3 amino acid changes with charged amino acids such that homodimer formation is electrostatically unfavorable are disclosed. EP1870459 and WO 2009089004 describe other strategies for favoring heterodimer formation upon co-expression of different antibody domains in a host cell. In these methods, one or more residues that make up the heavy chain constant domain 3 (CH3), CH3-CH3 interfaces in both CH3 domains are replaced with a charged amino acid such that homodimer formation is electrostatically unfavorable and heterodimerization is electrostatically favorable. Additional methods of making multispecific molecules using electrostatic interactions are described in the following references, the contents of each of which is incorporated by reference herein, include US20100015133, U.S. Pat. No. 8,592,562B2, U.S. Pat. No. 9,200,060B2, US20140154254A1, and U.S. Pat. No. 9,358,286A1.

### Common Light Chain

Light chain mispairing needs to be avoided to generate homogenous preparations of bispecific IgGs. One way to achieve this is through the use of the common light chain principle, i.e. combining two binders that share one light chain but still have separate specificities. An exemplary method of enhancing the formation of a desired bispecific antibody from a mixture of monomers is by providing a common variable light chain to interact with each of the heteromeric variable heavy chain regions of the bispecific antibody. Compositions and methods of producing bispecific antibodies with a common light chain as disclosed in, e.g., U.S. Pat. No. 7,183,076B2, US20110177073A1, EP2847231A1, WO2016079081A1, and EP3055329A1, the contents of each of which is incorporated by reference herein.

### Common Heavy Chain

An exemplary method of enhancing the formation of a desired bispecific antibody from a mixture of monomers is by providing a common variable heavy chain to interact with each of the heteromeric variable light chain regions of the bispecific antibody. Compositions and methods of producing bispecific antibodies with a common heavy chain are disclosed in, e.g., US20120184716, US20130317200, and US20160264685A1, the contents of each of which is incorporated by reference herein.

### CrossMab

Another option to reduce light chain mispairing is the CrossMab technology which avoids non-specific L chain mispairing by exchanging CH1 and CL domains in the Fab of one half of the bispecific antibody. Such crossover variants retain binding specificity and affinity, but make the two arms so different that L chain mispairing is prevented. The CrossMab technology (as reviewed in Klein et al. Supra) involves domain swapping between heavy and light chains so as to promote the formation of the correct pairings. Briefly, to construct a bispecific IgG-like CrossMab antibody that could bind to two antigens by using two distinct light chain-heavy chain pairs, a two-step modification process is applied. First, a dimerization interface is engineered into the C-terminus of each heavy chain using a heterodimerization approach, e.g., Knob-into-hole (KiH) technology, to ensure that only a heterodimer of two distinct heavy chains from one antibody (e.g., Antibody A) and a second antibody (e.g., Antibody B) is efficiently formed. Next, the constant heavy 1 (CH1) and constant light (CL) domains of one antibody are exchanged (Antibody A), keeping the variable heavy (VH) and variable light (VL) domains consistent. The exchange of the CH1 and CL domains ensured that the modified antibody (Antibody A) light chain would only efficiently dimerize with the modified antibody (antibody A) heavy chain, while the unmodified antibody (Antibody B) light chain would only efficiently dimerize with the unmodified antibody (Antibody B) heavy chain; and thus only the desired bispecific CrossMab would be efficiently formed (see e.g., Cain, C. SciBX 4(28); doi:10.1038/scibx.2011.783, the contents of which are incorporated by reference herein).

### Amino Acid Modifications

Alternative compositions and methods of producing multispecific antibodies with correct light chain pairing include various amino acid modifications. For example, Zymeworks describes heterodimers with one or more amino acid modifications in the CH1 and/or CL domains, one or more amino acid modifications in the VH and/or VL domains, or a combination thereof, which are part of the interface between the light chain and heavy chain and create preferential pairing between each heavy chain and a desired light chain such that when the two heavy chains and two light chains of the heterodimer pair are co-expressed in a cell, the heavy chain of the first heterodimer preferentially pairs with one of the light chains rather than the other (see e.g., WO2015181805). Other exemplary methods are described in WO2016026943 (Argen-X), US20150211001, US20140072581A1, US20160039947A1, and US20150368352.

### Lambda/Kappa Formats

Multispecific molecules (e.g., multispecific antibody molecules) that include the lambda light chain polypeptide and a kappa light chain polypeptide, can be used to allow for heterodimerization. Methods for generating bispecific antibody molecules comprising the lambda light chain polypeptide and a kappa light chain polypeptide are disclosed in PCT/US17/53053 filed on Sep. 22, 2017, incorporated herein by reference in its entirety.

In embodiments, the multispecific molecules includes a multispecific antibody molecule, e.g., an antibody molecule comprising two binding specificities, e.g., a bispecific antibody molecule. The multispecific antibody molecule includes:
a lambda light chain polypeptide 1 (LLCP1) specific for a first epitope;
a heavy chain polypeptide 1 (HCP1) specific for the first epitope;
a kappa light chain polypeptide 2 (KLCP2) specific for a second epitope; and
a heavy chain polypeptide 2 (HCP2) specific for the second epitope.

“Lambda light chain polypeptide 1 (LLCP1)”, as that term is used herein, refers to a polypeptide comprising sufficient light chain (LC) sequence, such that when combined with a cognate heavy chain variable region, can mediate specific binding to its epitope and complex with an HCP1. In an embodiment it comprises all or a fragment of a CH1 region. In an embodiment, an LLCP1 comprises LC-CDR1, LC-CDR2, LC-CDR3, FR1, FR2, FR3, FR4, and CH1, or sufficient sequence therefrom to mediate specific binding of its epitope and complex with an HCP1. LLCP1, together with its HCP1, provide specificity for a first epitope (while KLCP2, together with its HCP2, provide specificity for a second epitope). As described elsewhere herein, LLCP1 has a higher affinity for HCP1 than for HCP2.

“Kappa light chain polypeptide 2 (KLCP2)”, as that term is used herein, refers to a polypeptide comprising sufficient light chain (LC) sequence, such that when combined with a cognate heavy chain variable region, can mediate specific binding to its epitope and complex with an HCP2. In an embodiment, it comprises all or a fragment of a CH1 region. In an embodiment, a KLCP2 comprises LC-CDR1, LC-CDR2, LC-CDR3, FR1, FR2, FR3, FR4, and CH1, or sufficient sequence therefrom to mediate specific binding of its epitope and complex with an HCP2. KLCP2, together with its HCP2, provide specificity for a second epitope (while LLCP1, together with its HCP1, provide specificity for a first epitope).

“Heavy chain polypeptide 1 (HCP1)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In an embodiment, it comprises all or a fragment of a CH1 region. In an embodiment, it comprises all or a fragment of a CH2 and/or CH3 region. In an embodiment an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an LLCP1, (ii) to complex preferentially, as described herein to LLCP1 as opposed to KLCP2; and (iii) to complex preferentially, as described herein, to an HCP2, as opposed to another molecule of HCP1. HCP1, together with its LLCP1, provide specificity for a first epitope (while KLCP2, together with its HCP2, provide specificity for a second epitope).

“Heavy chain polypeptide 2 (HCP2)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In an embodiment, it comprises all or a fragment of a CH1 region. In an embodiment, it comprises all or a fragment of a CH2 and/or CH3 region. In an embodiment an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an KLCP2, (ii) to complex preferentially, as described herein to KLCP2 as opposed to LLCP1; and (iii) to complex preferentially, as described herein, to an HCP1, as opposed to another molecule of HCP2. HCP2, together with its KLCP2, provide specificity for a second epitope (while LLCP1, together with its HCP1, provide specificity for a first epitope).

In some embodiments of the multispecific antibody molecule disclosed herein:
LLCP1 has a higher affinity for HCP1 than for HCP2; and/or
KLCP2 has a higher affinity for HCP2 than for HCP1.

In embodiments, the affinity of LLCP1 for HCP1 is sufficiently greater than its affinity for HCP2, such that under preselected conditions, e.g., in aqueous buffer, e.g., at pH 7, in saline, e.g., at pH 7, or under physiological conditions, at least 75, 80, 90, 95, 98, 99, 99.5, or 99.9% of the multispecific antibody molecule molecules have a LLCP1 complexed, or interfaced with, a HCP1.

In some embodiments of the multispecific antibody molecule disclosed herein: the HCP1 has a greater affinity for HCP2, than for a second molecule of HCP1; and/or the HCP2 has a greater affinity for HCP1, than for a second molecule of HCP2.

In embodiments, the affinity of HCP1 for HCP2 is sufficiently greater than its affinity for a second molecule of HCP1, such that under preselected conditions, e.g., in aqueous buffer, e.g., at pH 7, in saline, e.g., at pH 7, or under physiological conditions, at least 75%, 80, 90, 95, 98, 99 99.5 or 99.9% of the multispecific antibody molecule molecules have a HCP1 complexed, or interfaced with, a HCP2.

In another aspect, disclosed herein is a method for making, or producing, a multispecific antibody molecule. The method includes:
(i) providing a first heavy chain polypeptide (e.g., a heavy chain polypeptide comprising one, two, three or all of a first heavy chain variable region (first VH), a first CH1, a first heavy chain constant region (e.g., a first CH2, a first CH3, or both));
(ii) providing a second heavy chain polypeptide (e.g., a heavy chain polypeptide comprising one, two, three or all of a second heavy chain variable region (second VH), a second CH1, a second heavy chain constant region (e.g., a second CH2, a second CH3, or both));
(iii) providing a lambda chain polypeptide (e.g., a lambda light variable region (VLλ), a lambda light constant chain (VLλ), or both) that preferentially associates with the first heavy chain polypeptide (e.g., the first VH); and
(iv) providing a kappa chain polypeptide (e.g., a kappa light variable region (VLκ), a kappa light constant chain (VLκ), or both) that preferentially associates with the second heavy chain polypeptide (e.g., the second VH), under conditions where (i)-(iv) associate.

In embodiments, the first and second heavy chain polypeptides form an Fe interface that enhances heterodimerization.

In embodiments, (i)-(iv) (e.g., nucleic acid encoding (i)-(iv)) are introduced in a single cell, e.g., a single mammalian cell, e.g., a CHO cell. In embodiments, (i)-(iv) are expressed in the cell.

In embodiments, (i)-(iv) (e.g., nucleic acid encoding (i)-(iv)) are introduced in different cells, e.g., different mammalian cells, e.g., two or more CHO cell. In embodiments, (i)-(iv) are expressed in the cells.

In one embodiment, the method further comprises purifying a cell-expressed antibody molecule, e.g., using a lambda- and/or- kappa-specific purification, e.g., affinity chromatography.

In embodiments, the method further comprises evaluating the cell-expressed multispecific antibody molecule. For example, the purified cell-expressed multispecific antibody molecule can be analyzed by techniques known in the art, include mass spectrometry. In one embodiment, the purified cell-expressed antibody molecule is cleaved, e.g., digested with papain to yield the Fab moieties and evaluated using mass spectrometry.

In embodiments, the method produces correctly paired kappa/lambda multispecific, e.g., bispecific, antibody molecules in a high yield, e.g., at least 75%, 80, 90, 95, 98, 99 99.5 or 99.9%.

In other embodiments, the multispecific, e.g., a bispecific, antibody molecule that includes: (i) a first heavy chain polypeptide (HCP1) (e.g., a heavy chain polypeptide comprising one, two, three or all of a first heavy chain variable region (first VH), a first CH1, a first heavy chain constant region (e.g., a first CH2, a first CH3, or both)), e.g., wherein the HCP1 binds to a first epitope;
(ii) a second heavy chain polypeptide (HCP2) (e.g., a heavy chain polypeptide comprising one, two, three or all of a second heavy chain variable region (second VH), a second CH1, a second heavy chain constant region (e.g., a second CH2, a second CH3, or both)), e.g., wherein the HCP2 binds to a second epitope;
(iii) a lambda light chain polypeptide (LLCP1) (e.g., a lambda light variable region (VLλ), a lambda light constant chain (VLλ), or both) that preferentially associates with the first heavy chain polypeptide (e.g., the first VH), e.g., wherein the LLCP1 binds to a first epitope; and
(iv) a kappa light chain polypeptide (KLCP2) (e.g., a kappa light variable region (VLκ), a kappa light constant chain (VLκ), or both) that preferentially associates with the second heavy chain polypeptide (e.g., the second VH), e.g., wherein the KLCP2 binds to a second epitope.

In embodiments, the first and second heavy chain polypeptides form an Fe interface that enhances heterodimerization. In embodiments, the multispecific antibody molecule has a first binding specificity that includes a hybrid VLλ-CLλ heterodimerized to a first heavy chain variable region connected to the Fc constant, CH2-CH3 domain (having a knob modification) and a second binding specificity that includes a hybrid VLκ-CLκ heterodimerized to a second heavy chain variable region connected to the Fc constant, CH2-CH3 domain (having a hole modification).

### TRBC1 and TRBC2 Antigen Binding Domains

The present disclosure provides, inter alia, antibody molecules, e.g., multispecific (e.g., bi-, tri-, tetra-specific) or multifunctional molecules, that include, e.g., are engineered to contain, one or more antigen binding domains that bind to a tumor antigen on a lymphoma cell (e.g., T cell). In some embodiments, the tumor antigen comprises a T cell receptor comprising TRBC1 or TRBC2. In some embodiments, the antigen binding domain preferentially binds to a T cell receptor comprising TRBC1 (e.g., relative to a T cell receptor comprising TRBC2). In some embodiments, the antigen binding domain preferentially binds to a T cell receptor comprising TRBC2 (e.g., relative to a T cell receptor comprising TRBC1). In some embodiments, the multifunctional molecules include, e.g., are engineered to contain, one or more antigen binding domains that selectively target lymphocytes expressing TRBC1 or TRBC2. In some embodiments, the antigen binding domain selectively targets lymphocytes expressing a T cell receptor comprising TRBC1 or a T cell receptor comprising TRBC2.

T cell receptors (TCRs) are receptors found on the surface of lymphocytes, specifically on T lymphocytes (T cells). TCRs are responsible for recognizing antigen fragments presented by major histocompatibility complex (MHC) molecules on other immune cells (e.g., B cells) by signaling through associated CD3 and activating the T cell. The vast majority of TCRs in humans are heterodimers comprising an alpha chain and a beta chain. Both alpha and beta chains of TCR comprise variable and constant regions. The variable regions of the alpha and beta chain are encoded by distinct DNA elements (V, D, and J elements for beta chain; V and J elements for the alpha chain). Recombination between these elements produces in large part the variation in antigen binding specificity of TCRs. The TCR beta chain constant region is selected from two different domains, beta constant domain 1 and beta constant domain 2. Without wishing to be bound by theory, it is thought that the majority of TCRs comprising a beta chain comprise a beta chain comprising beta constant domain 1 or beta constant domain 2, but not both constant domain 1 and constant domain 2.

In some embodiments, the multifunctional or multispecific molecules or antibody molecules of the present application comprise an antigen binding domain that binds to a tumor antigen on a lymphoma cell (e.g., a T cell), e.g., a T cell receptor comprising TRBC1, TRBC1, a T cell receptor comprising TRBC2, or TRBC2. In some embodiments, the multifunctional or multispecific molecules or antibody molecules of the present application comprise an antigen binding domain that selectively targets lymphocytes expressing a T cell receptor comprising TRBC1, TRBC1, a T cell receptor comprising TRBC2, or TRBC2. While it is most typical for a lymphocyte or lymphoma cell presenting a T cell receptor comprising TRBC1 or TRBC2 to be a T cell, cancer causes many disruptions in non-disease expression patterns. Thus, in some embodiments, the lymphoma cell or lymphocyte may not be a T cell. In some embodiments, the lymphoma cell or lymphocyte is a B cell. In some embodiments, the lymphoma cell or lymphocyte is a natural killer cell.

In some embodiments, the antigen binding domain (e.g., first antigen binding domain) comprises any CDR amino acid sequence, framework region (FWR) amino acid sequence, or variable region amino acid sequence of an anti-TRBC1 antibody known in the art. In some embodiments, CDR amino acid sequence, framework region (FWR) amino acid sequence, or variable region amino acid sequence are selected from JOVI.1.

### TRBC1 Antigen Binding Domains

In some embodiments, the antigen binding domain that binds to TRBC1 comprises one or more CDRs (e.g., VHCDR1, VHCDR2, VHCDR3, VLCDR1, VLCDR2, and/or VLCDR3) disclosed in Table 2A or Table 2B, Table 3A or Table 3B, or Table 4, or a sequence having at least 85%, 90%, 95%, or 99% identity thereto. In some embodiments, the antigen binding domain that binds to TRBC1 comprises one or more framework regions (e.g., VHFWR1, VHFWR2, VHFWR3, VHFWR4, VLFWR1, VLFWR2, VLFWR3, and/or VLFWR4) disclosed in Table 2A or Table 2B, Table 3A or Table 3B, or Table 4, or a sequence having at least 85%, 90%, 95%, or 99% identity thereto. In some embodiments, the antigen binding domain that binds to TRBC1 comprises a VH and/or a VL disclosed in Table 7, or a sequence having at least 85%, 90%, 95%, or 99% identity thereto. In some embodiments, the antigen binding domain that binds to TRBC1 comprises an amino acid sequence disclosed in Table 8, or a sequence having at least 85%, 90%, 95%, or 99% identity thereto.

In some embodiments, the antigen binding domain that binds to TRBC1 comprises one or more CDRs (e.g., VHCDR1, VHCDR2, VHCDR3, VLCDR1, VLCDR2, and/or VLCDR3) disclosed in Table 5A and/or 5B, or a sequence having at least 85%, 90%, 95%, or 99% identity thereto. In some embodiments, the antigen binding domain that binds to TRBC1 comprises one or more framework regions (e.g., VHFWR1, VHFWR2, VHFWR3, VHFWR4, VLFWR1, VLFWR2, VLFWR3, and/or VLFWR4) disclosed in Table 5A and/or 5B, or a sequence having at least 85%, 90%, 95%, or 99% identity thereto. In some embodiments, the antigen binding domain that binds to TRBC1 comprises a VH and/or a VL disclosed in Table 7, or a sequence having at least 85%, 90%, 95%, or 99% identity thereto.

In some embodiments, the antigen binding domain that binds to TRBC1 comprises a VH comprising a heavy chain complementarity determining region 1 (VHCDR1), a VHCDR2, and a VHCDR3, and a VL comprising a light chain complementarity determining region 1 (VLCDR1), a VLCDR2, and a VLCDR3.

In some embodiments, the VHCDR1, VHCDR2, and VHCDR3 comprise the amino acid sequences of SEQ ID NOs: 8643, 7355, and 202, respectively (or a sequence having at least 85%, 90%, 95%, or 99% identity thereto). In some embodiments, the VHCDR1, VHCDR2, and VHCDR3 comprise the amino acid sequences of SEQ ID NOs: 8643, 201, and 202, respectively (or a sequence having at least 85%, 90%, 95%, or 99% identity thereto). In some embodiments, the VHCDR1, VHCDR2, and VHCDR3 comprise the amino acid sequences of SEQ ID NOs: 7354, 201, and 202, respectively (or a sequence having at least 85%, 90%, 95%, or 99% identity thereto). In some embodiments, the VHCDR1, VHCDR2, and VHCDR3 comprise the amino acid sequences of SEQ ID NOs: 7354, 7355, and 202, respectively (or a sequence having at least 85%, 90%, 95%, or 99% identity thereto).

In some embodiments, the VLCDR1, VLCDR2, and VLCDR3 comprise the amino acid sequences of SEQ ID NOs: 223, 224, and 225 or 8680, respectively (or a sequence having at least 85%, 90%, 95%, or 99% identity thereto). In some embodiments, the VLCDR1, VLCDR2, and VLCDR3 comprise the amino acid sequences of SEQ ID NOs: 7367, 224, and 225 or 8680, respectively (or a sequence having at least 85%, 90%, 95%, or 99% identity thereto). In some embodiments, the VLCDR1, VLCDR2, and VLCDR3 comprise the amino acid sequences of SEQ ID NOs: 223, 7368, and 225 or 8680, respectively (or a sequence having at least 85%, 90%, 95%, or 99% identity thereto). In some embodiments, the VLCDR1, VLCDR2, and VLCDR3 comprise the amino acid sequences of SEQ ID NOs: 223, 224, and 7369, respectively (or a sequence having at least 85%, 90%, 95%, or 99% identity thereto). In some embodiments, the VLCDR1, VLCDR2, and VLCDR3 comprise the amino acid sequences of SEQ ID NOs: 7367, 7368, and 7369, respectively (or a sequence having at least 85%, 90%, 95%, or 99% identity thereto).

In some embodiments, the VHCDR1, VHCDR2, VHCDR3, VLCDR1, VLCDR2, and VLCDR3 comprise the amino acid sequences of SEQ ID NOs: 8643, 7355, 202, 223, 224, and 225 or 8680, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 8643, 201, 202, 223, 224, and 225 or 8680, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of: SEQ ID NOs: 8643, 7355, 202, 7367, 224, and 225 or 8680, respectively (or a sequence having at least 85%, 90%, 95%, or 99% identity thereto); SEQ ID NOs: 8643, 7355, 202, 223, 7368, and 225 or 8680, respectively (or a sequence having at least 85%, 90%, 95%, or 99% identity thereto); SEQ ID NOs: 8643, 7355, 202, 223, 224, and 7369, respectively (or a sequence having at least 85%, 90%, 95%, or 99% identity thereto); SEQ ID NOs: 8643, 7355, 202, 7367, 7368, and 7369, respectively (or a sequence having at least 85%, 90%, 95%, or 99% identity thereto); SEQ ID NOs: 8643, 201, 202, 7367, 224, and 225 or 8680, respectively (or a sequence having at least 85%, 90%, 95%, or 99% identity thereto); SEQ ID NOs: 8643, 201, 202, 223, 7368, and 225 or 8680, respectively (or a sequence having at least 85%, 90%, 95%, or 99% identity thereto); SEQ ID NOs: 8643, 201, 202, 223, 224, and 7369, respectively (or a sequence having at least 85%, 90%, 95%, or 99% identity thereto); SEQ ID NOs: 8643, 201, 202, 7367, 7368, and 7369, respectively (or a sequence having at least 85%, 90%, 95%, or 99% identity thereto); SEQ ID NOs: 7354, 201, 202, 223, 224, and 225 or 8680, respectively (or a sequence having at least 85%, 90%, 95%, or 99% identity thereto); SEQ ID NOs: 7354, 201, 202, 7367, 224, and 225 or 8680, respectively (or a sequence having at least 85%, 90%, 95%, or 99% identity thereto); SEQ ID NOs: 7354, 201, 202, 223, 7368, and 225 or 8680, respectively (or a sequence having at least 85%, 90%, 95%, or 99% identity thereto); SEQ ID NOs: 7354, 201, 202, 223, 224, and 7369, respectively (or a sequence having at least 85%, 90%, 95%, or 99% identity thereto); SEQ ID NOs: 7354, 201, 202, 7367, 7368, and 7369, respectively (or a sequence having at least 85%, 90%, 95%, or 99% identity thereto); SEQ ID NOs: 7354, 7355, 202, 223, 224, and 225 or 8680, respectively (or a sequence having at least 85%, 90%, 95%, or 99% identity thereto); SEQ ID NOs: 7354, 7355, 202, 7367, 224, and 225 or 8680, respectively (or a sequence having at least 85%, 90%, 95%, or 99% identity thereto); SEQ ID NOs: 7354, 7355, 202, 223, 7368, and 225 or 8680, respectively (or a sequence having at least 85%, 90%, 95%, or 99% identity thereto); SEQ ID NOs: 7354, 7355, 202, 223, 224, and 7369, respectively (or a sequence having at least 85%, 90%, 95%, or 99% identity thereto); or SEQ ID NOs: 7354, 7355, 202, 7367, 7368, and 7369, respectively (or a sequence having at least 85%, 90%, 95%, or 99% identity thereto).

In some embodiments, the VH comprises an amino acid sequence selected from the group consisting of SEQ ID NOs: 7351, 253, 250-252, 254, 7343, 7344, 7350, and 7352 (or a sequence having at least 85%, 90%, 95%, or 99% identity thereto) and/or the VL comprises an amino acid sequence selected from the group consisting of SEQ ID NOs: 258, 255-257, 259, 260, and 7357-7360 (or a sequence having at least 85%, 90%, 95%, or 99% identity thereto). In some embodiments, the VH and VL comprise the amino acid sequences of SEQ ID NOs: 7351 and 258, respectively (or a sequence having at least 85%, 90%, 95%, or 99% identity thereto). In some embodiments, the VH and VL comprise the amino acid sequences of SEQ ID NOs: 253 and 258, respectively (or a sequence having at least 85%, 90%, 95%, or 99% identity thereto).

In some embodiments, the antigen binding domain (e.g., first antigen binding domain) that binds to a tumor antigen on a lymphoma cell (e.g., a T cell), e.g., a T cell receptor comprising TRBC1, TRBC1, a T cell receptor comprising TRBC2, or TRBC2 comprises any CDR amino acid sequence, framework region (FWR) amino acid sequence, or variable region amino acid sequence Table 1, Table 2A or Table 2B, Table 3A or Table 3B, Table 4, Table 7, and Table 8. In some embodiments, the antigen binding domain (e.g., first antigen binding domain) that binds to a tumor antigen on a lymphoma cell (e.g., a T cell), e.g., a T cell receptor comprising TRBC1, TRBC1, a T cell receptor comprising TRBC2, or TRBC2 comprises heavy and/or light chain amino acid sequences of Table 8. In some embodiments, the antigen binding domain (e.g., first antigen binding domain) that selectively targets lymphocytes expressing a T cell receptor comprising TRBC1, TRBC1, a T cell receptor comprising TRBC2, or TRBC2 comprises any CDR amino acid sequence, framework region (FWR) amino acid sequence, or variable region amino acid sequence disclosed in Table 1, Table 2A or Table 2B, Table 3A or Table 3B, Table 4, Table 7, and Table 8. In some embodiments, the antigen binding domain (e.g., first antigen binding domain) that selectively targets lymphocytes expressing a T cell receptor comprising TRBC1, TRBC1, a T cell receptor comprising TRBC2, or TRBC2 comprises heavy and/or light chain amino acid sequences of Table 8. An antigen binding domain that binds to a tumor antigen comprising TRBC1 or selectively targets lymphocytes expressing TRBC1 may be said to target TRBC1 (i.e., a TRBC1-targeting antigen binding domain). An antigen binding domain that binds to a tumor antigen comprising TRBC2 or selectively targets lymphocytes expressing TRBC2 may be said to target TRBC2 (i.e., a TRBC2-targeting antigen binding domain).

In some embodiments, the antigen binding domain that targets TRBC1 comprises a VH comprising a heavy chain complementarity determining region 1 (VHCDR1) amino acid sequence of SEQ ID NO: 8643 (or a sequence with no more than 1, 2, 3, or 4 mutations, e.g., substitutions, additions, or deletions), a VHCDR2 amino acid sequence of SEQ ID NO: 201 (or a sequence with no more than 1, 2, 3, or 4 mutations, e.g., substitutions, additions, or deletions), and/or a VHCDR3 amino acid sequence of SEQ ID NO: 202 (or a sequence with no more than 1, 2, 3, or 4 mutations, e.g., substitutions, additions, or deletions). In some embodiments, the TRBC1 antigen binding domain comprises a VH comprising a VHCDR1 amino acid sequence of SEQ ID NO: 8643, a VHCDR2 amino acid sequence of SEQ ID NO: 201, and/or a VHCDR3 amino acid sequence of SEQ ID NO: 202.

In some embodiments, the antigen binding domain that targets TRBC1 comprises a VL comprising a light chain complementarity determining region 1 (VLCDR1) amino acid sequence of SEQ ID NO: 223 (or a sequence with no more than 1, 2, 3, or 4 mutations, e.g., substitutions, additions, or deletions), a VLCDR2 amino acid sequence of SEQ ID NO: 224 (or a sequence with no more than 1, 2, 3, or 4 mutations, e.g., substitutions, additions, or deletions), and/or a VLCDR3 amino acid sequence of SEQ ID NO: 225 or 8680 (or a sequence with no more than 1, 2, 3, or 4 mutations, e.g., substitutions, additions, or deletions). In some embodiments, the antigen binding domain that targets TRBC1 comprises a VL comprising a VLCDR1 amino acid sequence of SEQ ID NO: 223, a VLCDR2 amino acid sequence of SEQ ID NO: 224, and a VLCDR3 amino acid sequence of SEQ ID NO: 225 or 8680.

In some embodiments, the antigen binding domain that targets TRBC1 comprises a VH comprising a heavy chain framework region 1 (VHFWR1) amino acid sequence of SEQ ID NO: 203, a VHFWR2 amino acid sequence of SEQ ID NO: 204, a VHFWR3 amino acid sequence of SEQ ID NO: 8679, and/or a VHFWR4 amino acid sequence of SEQ ID NO: 206.

In some embodiments, the antigen binding domain that targets TRBC1 comprises a VL comprising a light chain framework region 1 (VLFWR1) amino acid sequence of SEQ ID NO: 226, a VLFWR2 amino acid sequence of SEQ ID NO: 227, a VLFWR3 amino acid sequence of SEQ ID NO: 228, and/or a VLFWR4 amino acid sequence of SEQ ID NO: 229.

In some embodiments, the antigen binding domain that targets TRBC1 comprises a VH comprising a VHFWR1 amino acid sequence of SEQ ID NO: 203 (or a sequence with no more than 1, 2, 3, 4, 5, or 6 mutations, e.g., substitutions, additions, or deletions, therefrom), a VHFWR2 amino acid sequence of SEQ ID NO: 204 (or a sequence with no more than 1, 2, 3, 4, 5, or 6 mutations, e.g., substitutions, additions, or deletions, therefrom), a VHFWR3 amino acid sequence of SEQ ID NO: 8679 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 206.

In some embodiments, the antigen binding domain that targets TRBC1 comprises a VL comprising a VLFWR1 amino acid sequence of SEQ ID NO: 226 (or a sequence with no more than 1, 2, or 3 mutations, e.g., substitutions, additions, or deletions), a VLFWR2 amino acid sequence of SEQ ID NO: 227 (or a sequence with no more than 1 mutation, e.g., substitution, addition, or deletion), a VLFWR3 amino acid sequence of SEQ ID NO: 228 (or a sequence with no more than 1 mutation, e.g., substitution, addition, or deletion), and/or a VLFWR4 amino acid sequence of SEQ ID NO: 229.

In some embodiments, the antigen binding domain that targets TRBC1 comprises a VH comprising a heavy chain framework region 1 (VHFWR1) amino acid sequence of SEQ ID NO: 207, a VHFWR2 amino acid sequence of SEQ ID NO: 208, a VHFWR3 amino acid sequence of SEQ ID NO: 209, and/or a VHFWR4 amino acid sequence of SEQ ID NO: 210.

In some embodiments, the antigen binding domain that targets TRBC1 comprises a VH comprising a VHFWR1 amino acid sequence of SEQ ID NO: 207 (or a sequence with no more than 1, 2, 3, 4, 5, or 6 mutations, e.g., substitutions, additions, or deletions, therefrom), a VHFWR2 amino acid sequence of SEQ ID NO: 208 (or a sequence with no more than 1, 2, 3, 4, 5, or 6 mutations, e.g., substitutions, additions, or deletions, therefrom), a VHFWR3 amino acid sequence of SEQ ID NO: 209 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 210.

In some embodiments, the antigen binding domain that targets TRBC1 comprises a VH comprising a heavy chain framework region 1 (VHFWR1) amino acid sequence of SEQ ID NO: 207, a VHFWR2 amino acid sequence of SEQ ID NO: 208, a VHFWR3 amino acid sequence of SEQ ID NO: 213, and/or a VHFWR4 amino acid sequence of SEQ ID NO: 210.

In some embodiments, the antigen binding domain that targets TRBC1 comprises a VH comprising a VHFWR1 amino acid sequence of SEQ ID NO: 207 (or a sequence with no more than 1, 2, 3, 4, 5, or 6 mutations, e.g., substitutions, additions, or deletions, therefrom), a VHFWR2 amino acid sequence of SEQ ID NO: 208 (or a sequence with no more than 1, 2, 3, 4, 5, or 6 mutations, e.g., substitutions, additions, or deletions, therefrom), a VHFWR3 amino acid sequence of SEQ ID NO: 213 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 210.

In some embodiments, the antigen binding domain that targets TRBC1 comprises a VH comprising a heavy chain framework region 1 (VHFWR1) amino acid sequence of SEQ ID NO: 7345, a VHFWR2 amino acid sequence of SEQ ID NO: 208, a VHFWR3 amino acid sequence of SEQ ID NO: 217, and/or a VHFWR4 amino acid sequence of SEQ ID NO: 210.

In some embodiments, the antigen binding domain that targets TRBC1 comprises a VH comprising a VHFWR1 amino acid sequence of SEQ ID NO: 7345 (or a sequence with no more than 1, 2, 3, 4, 5, or 6 mutations, e.g., substitutions, additions, or deletions, therefrom), a VHFWR2 amino acid sequence of SEQ ID NO: 208 (or a sequence with no more than 1, 2, 3, 4, 5, or 6 mutations, e.g., substitutions, additions, or deletions, therefrom), a VHFWR3 amino acid sequence of SEQ ID NO: 217 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 210.

In some embodiments, the antigen binding domain that targets TRBC1 comprises a VH comprising a heavy chain framework region 1 (VHFWR1) amino acid sequence of SEQ ID NO: 207, a VHFWR2 amino acid sequence of SEQ ID NO: 212, a VHFWR3 amino acid sequence of SEQ ID NO: 221, and/or a VHFWR4 amino acid sequence of SEQ ID NO: 210.

In some embodiments, the antigen binding domain that targets TRBC1 comprises a VH comprising a VHFWR1 amino acid sequence of SEQ ID NO: 207 (or a sequence with no more than 1, 2, 3, 4, 5, or 6 mutations, e.g., substitutions, additions, or deletions, therefrom), a VHFWR2 amino acid sequence of SEQ ID NO: 212 (or a sequence with no more than 1, 2, 3, 4, 5, or 6 mutations, e.g., substitutions, additions, or deletions, therefrom), a VHFWR3 amino acid sequence of SEQ ID NO: 221 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 210.

In some embodiments, the antigen binding domain that targets TRBC1 comprises a VL comprising a light chain framework region 1 (VLFWR1) amino acid sequence of SEQ ID NO: 230, a VLFWR2 amino acid sequence of SEQ ID NO: 231, a VLFWR3 amino acid sequence of SEQ ID NO: 232, and/or a VLFWR4 amino acid sequence of SEQ ID NO: 8685.

In some embodiments, the antigen binding domain that targets TRBC1 comprises a VL comprising a VLFWR1 amino acid sequence of SEQ ID NO: 230 (or a sequence with no more than 1, 2, or 3 mutations, e.g., substitutions, additions, or deletions), a VLFWR2 amino acid sequence of SEQ ID NO: 231 (or a sequence with no more than 1 mutation, e.g., substitution, addition, or deletion), a VLFWR3 amino acid sequence of SEQ ID NO: 232 (or a sequence with no more than 1 mutation, e.g., substitution, addition, or deletion), and/or a VLFWR4 amino acid sequence of SEQ ID NO: 8685.

In some embodiments, the antigen binding domain that targets TRBC1 comprises a VL comprising a light chain framework region 1 (VLFWR1) amino acid sequence of SEQ ID NO: 234, a VLFWR2 amino acid sequence of SEQ ID NO: 235, a VLFWR3 amino acid sequence of SEQ ID NO: 236, and/or a VLFWR4 amino acid sequence of SEQ ID NO: 237.

In some embodiments, the antigen binding domain that targets TRBC1 comprises a VL comprising a VLFWR1 amino acid sequence of SEQ ID NO: 234 (or a sequence with no more than 1, 2, or 3 mutations, e.g., substitutions, additions, or deletions), a VLFWR2 amino acid sequence of SEQ ID NO: 235 (or a sequence with no more than 1 mutation, e.g., substitution, addition, or deletion), a VLFWR3 amino acid sequence of SEQ ID NO: 236 (or a sequence with no more than 1 mutation, e.g., substitution, addition, or deletion), and/or a VLFWR4 amino acid sequence of SEQ ID NO: 237.

In some embodiments, the antigen binding domain that targets TRBC1 comprises a VL comprising a light chain framework region 1 (VLFWR1) amino acid sequence of SEQ ID NO: 238, a VLFWR2 amino acid sequence of SEQ ID NO: 239, a VLFWR3 amino acid sequence of SEQ ID NO: 240, and/or a VLFWR4 amino acid sequence of SEQ ID NO: 241.

In some embodiments, the antigen binding domain that targets TRBC1 comprises a VL comprising a VLFWR1 amino acid sequence of SEQ ID NO: 238 (or a sequence with no more than 1, 2, or 3 mutations, e.g., substitutions, additions, or deletions), a VLFWR2 amino acid sequence of SEQ ID NO: 239 (or a sequence with no more than 1 mutation, e.g., substitution, addition, or deletion), a VLFWR3 amino acid sequence of SEQ ID NO: 240 (or a sequence with no more than 1 mutation, e.g., substitution, addition, or deletion), and/or a VLFWR4 amino acid sequence of SEQ ID NO: 241.

In some embodiments, the antigen binding domain that targets TRBC1 comprises a VL comprising a light chain framework region 1 (VLFWR1) amino acid sequence of SEQ ID NO: 242, a VLFWR2 amino acid sequence of SEQ ID NO: 243, a VLFWR3 amino acid sequence of SEQ ID NO: 240, and/or a VLFWR4 amino acid sequence of SEQ ID NO: 245.

In some embodiments, the antigen binding domain that targets TRBC1 comprises a VL comprising a VLFWR1 amino acid sequence of SEQ ID NO: 242 (or a sequence with no more than 1, 2, or 3 mutations, e.g., substitutions, additions, or deletions), a VLFWR2 amino acid sequence of SEQ ID NO: 243 (or a sequence with no more than 1 mutation, e.g., substitution, addition, or deletion), a VLFWR3 amino acid sequence of SEQ ID NO: 240 (or a sequence with no more than 1 mutation, e.g., substitution, addition, or deletion), and/or a VLFWR4 amino acid sequence of SEQ ID NO: 245.

In some embodiments, the antigen binding domain that targets TRBC1 comprises a VL comprising a light chain framework region 1 (VLFWR1) amino acid sequence of SEQ ID NO: 246, a VLFWR2 amino acid sequence of SEQ ID NO: 247, a VLFWR3 amino acid sequence of SEQ ID NO: 240, and/or a VLFWR4 amino acid sequence of SEQ ID NO: 249.

In some embodiments, the antigen binding domain that targets TRBC1 comprises a VL comprising a VLFWR1 amino acid sequence of SEQ ID NO: 246 (or a sequence with no more than 1, 2, or 3 mutations, e.g., substitutions, additions, or deletions), a VLFWR2 amino acid sequence of SEQ ID NO: 247 (or a sequence with no more than 1 mutation, e.g., substitution, addition, or deletion), a VLFWR3 amino acid sequence of SEQ ID NO: 240 (or a sequence with no more than 1 mutation, e.g., substitution, addition, or deletion), and/or a VLFWR4 amino acid sequence of SEQ ID NO: 249.

In some embodiments, the antigen binding domain that targets TRBC1 comprises a VH comprising the amino acid sequence of SEQ ID NO: 250 (or an amino acid sequence having at least about 77%, 80%, 85%, 90%, 95%, or 99% sequence identity to SEQ ID NO: 250). In some embodiments, the antigen binding domain that targets TRBC1 comprises a VL comprising the amino acid sequence of SEQ ID NO: 255 (or an amino acid sequence having at least about 93%, 95%, or 99% sequence identity to SEQ ID NO: 255). In some embodiments, antigen binding domain that targets TRBC1 comprises a VH comprising the amino acid sequence of SEQ ID NO: 250. In some embodiments, the antigen binding domain that targets TRBC1 comprises a VL comprising the amino acid sequence of SEQ ID NO: 255.

In some embodiments, the antigen binding domain that targets TRBC1 comprises a VH comprising the amino acid sequence of SEQ ID NO: 250, and a VL comprising the amino acid sequence of SEQ ID NO: 255.

In some embodiments, the antigen binding domain that targets TRBC1 comprises a VH comprising the amino acid sequence of SEQ ID NO: 251 (or an amino acid sequence having at least about 77%, 80%, 85%, 90%, 95%, or 99% sequence identity to SEQ ID NO: 251). In some embodiments, antigen binding domain that targets TRBC1 comprises a VH comprising the amino acid sequence of SEQ ID NO: 251.

In some embodiments, the antigen binding domain that targets TRBC1 comprises a VH comprising the amino acid sequence of SEQ ID NO: 252 (or an amino acid sequence having at least about 77%, 80%, 85%, 90%, 95%, or 99% sequence identity to SEQ ID NO: 252). In some embodiments, antigen binding domain that targets TRBC1 comprises a VH comprising the amino acid sequence of SEQ ID NO: 252.

In some embodiments, the antigen binding domain that targets TRBC1 comprises a VH comprising the amino acid sequence of SEQ ID NO: 253 (or an amino acid sequence having at least about 77%, 80%, 85%, 90%, 95%, or 99% sequence identity to SEQ ID NO: 253). In some embodiments, antigen binding domain that targets TRBC1 comprises a VH comprising the amino acid sequence of SEQ ID NO: 253.

In some embodiments, the antigen binding domain that targets TRBC1 comprises a VH comprising the amino acid sequence of SEQ ID NO: 254 (or an amino acid sequence having at least about 77%, 80%, 85%, 90%, 95%, or 99% sequence identity to SEQ ID NO: 254). In some embodiments, antigen binding domain that targets TRBC1 comprises a VH comprising the amino acid sequence of SEQ ID NO: 254.

In some embodiments, the antigen binding domain that targets TRBC1 comprises a VL comprising the amino acid sequence of SEQ ID NO: 256 (or an amino acid sequence having at least about 93%, 95%, or 99% sequence identity to SEQ ID NO: 256). In some embodiments, the antigen binding domain that targets TRBC1 comprises a VL comprising the amino acid sequence of SEQ ID NO: 256.

In some embodiments, the antigen binding domain that targets TRBC1 comprises a VL comprising the amino acid sequence of SEQ ID NO: 257 (or an amino acid sequence having at least about 93%, 95%, or 99% sequence identity to SEQ ID NO: 257). In some embodiments, the antigen binding domain that targets TRBC1 comprises a VL comprising the amino acid sequence of SEQ ID NO: 257.

In some embodiments, the antigen binding domain that targets TRBC1 comprises a VL comprising the amino acid sequence of SEQ ID NO: 258 (or an amino acid sequence having at least about 93%, 95%, or 99% sequence identity to SEQ ID NO: 258). In some embodiments, the antigen binding domain that targets TRBC1 comprises a VL comprising the amino acid sequence of SEQ ID NO: 258.

In some embodiments, the antigen binding domain that targets TRBC1 comprises a VL comprising the amino acid sequence of SEQ ID NO: 259 (or an amino acid sequence having at least about 93%, 95%, or 99% sequence identity to SEQ ID NO: 259). In some embodiments, the antigen binding domain that targets TRBC1 comprises a VL comprising the amino acid sequence of SEQ ID NO: 259.

In some embodiments, the antigen binding domain that targets TRBC1 comprises a VL comprising the amino acid sequence of SEQ ID NO: 260 (or an amino acid sequence having at least about 93%, 95%, or 99% sequence identity to SEQ ID NO: 260). In some embodiments, the antigen binding domain that targets TRBC1 comprises a VL comprising the amino acid sequence of SEQ ID NO: 260.

In some embodiments, the antigen binding domain that targets TRBC1 comprises a heavy chain comprising the amino acid sequence of SEQ ID NO: 6154 (or an amino acid sequence having at least about 93%, 95%, or 99% sequence identity to SEQ ID NO: 6154). In some embodiments, the antigen binding domain that targets TRBC1 comprises a heavy chain comprising the amino acid sequence of SEQ ID NO: 6154.

In some embodiments, the antigen binding domain that targets TRBC1 comprises a heavy chain comprising the amino acid sequence of SEQ ID NO: 4671 (or an amino acid sequence having at least about 93%, 95%, or 99% sequence identity to SEQ ID NO: 4671). In some embodiments, the antigen binding domain that targets TRBC1 comprises a heavy chain comprising the amino acid sequence of SEQ ID NO: 4671.

In some embodiments, the antigen binding domain that targets TRBC1 comprises a heavy chain comprising the amino acid sequence of SEQ ID NO: 6155 (or an amino acid sequence having at least about 93%, 95%, or 99% sequence identity to SEQ ID NO: 6155). In some embodiments, the antigen binding domain that targets TRBC1 comprises a heavy chain comprising the amino acid sequence of SEQ ID NO: 6155.

In some embodiments, the antigen binding domain that targets TRBC1 comprises a heavy chain comprising the amino acid sequence of SEQ ID NO: 4672 (or an amino acid sequence having at least about 93%, 95%, or 99% sequence identity to SEQ ID NO: 4672). In some embodiments, the antigen binding domain that targets TRBC1 comprises a heavy chain comprising the amino acid sequence of SEQ ID NO: 4672.

In some embodiments, the antigen binding domain that targets TRBC1 comprises a light chain comprising the amino acid sequence of SEQ ID NO: 6156 (or an amino acid sequence having at least about 93%, 95%, or 99% sequence identity to SEQ ID NO: 6156). In some embodiments, the antigen binding domain that targets TRBC1 comprises a light chain comprising the amino acid sequence of SEQ ID NO: 6156.

In some embodiments, the antigen binding domain that targets TRBC1 comprises a heavy chain comprising the amino acid sequence of SEQ ID NO: 6167 (or an amino acid sequence having at least about 93%, 95%, or 99% sequence identity to SEQ ID NO: 6167). In some embodiments, the antigen binding domain that targets TRBC1 comprises a heavy chain comprising the amino acid sequence of SEQ ID NO: 6167.

In some embodiments, the antigen binding domain that targets TRBC1 comprises a heavy chain comprising the amino acid sequence of SEQ ID NO: 4673 (or an amino acid sequence having at least about 93%, 95%, or 99% sequence identity to SEQ ID NO: 4673). In some embodiments, the antigen binding domain that targets TRBC1 comprises a heavy chain comprising the amino acid sequence of SEQ ID NO: 4673.

In some embodiments, the antigen binding domain that targets TRBC1 comprises a heavy chain comprising the amino acid sequence of SEQ ID NO: 6168 (or an amino acid sequence having at least about 93%, 95%, or 99% sequence identity to SEQ ID NO: 6168). In some embodiments, the antigen binding domain that targets TRBC1 comprises a heavy chain comprising the amino acid sequence of SEQ ID NO: 6168. [0505]n some embodiments, the antigen binding domain that targets TRBC1 comprises a heavy chain comprising the amino acid sequence of SEQ ID NO: 4674 (or an amino acid sequence having at least about 93%, 95%, or 99% sequence identity to SEQ ID NO: 4674). In some embodiments, the antigen binding domain that targets TRBC1 comprises a heavy chain comprising the amino acid sequence of SEQ ID NO: 4674.

In some embodiments, the antigen binding domain that targets TRBC1 comprises a light chain comprising the amino acid sequence of SEQ ID NO: 6169 (or an amino acid sequence having at least about 93%, 95%, or 99% sequence identity to SEQ ID NO: 6169). In some embodiments, the antigen binding domain that targets TRBC1 comprises a light chain comprising the amino acid sequence of SEQ ID NO: 6169.

In some embodiments, the antigen binding domain that targets TRBC1 comprises a heavy chain comprising the amino acid sequence of SEQ ID NO: 6154 (or an amino acid sequence having at least about 93%, 95%, or 99% sequence identity to SEQ ID NO: 6154) and a light chain comprising the amino acid sequence of SEQ ID NO: 6156 (or an amino acid sequence having at least about 93%, 95%, or 99% sequence identity to SEQ ID NO: 6156). In some embodiments, the antigen binding domain that targets TRBC1 comprises a heavy chain comprising the amino acid sequence of SEQ ID NO: 6154 and a light chain comprising the amino acid sequence of SEQ ID NO: 6156.

In some embodiments, the antigen binding domain that targets TRBC1 comprises a heavy chain comprising the amino acid sequence of SEQ ID NO: 6155 (or an amino acid sequence having at least about 93%, 95%, or 99% sequence identity to SEQ ID NO: 6155) and a light chain comprising the amino acid sequence of SEQ ID NO: 6156 (or an amino acid sequence having at least about 93%, 95%, or 99% sequence identity to SEQ ID NO: 6156). In some embodiments, the antigen binding domain that targets TRBC1 comprises a heavy chain comprising the amino acid sequence of SEQ ID NO: 6155 and a light chain comprising the amino acid sequence of SEQ ID NO: 6156.

In some embodiments, the antigen binding domain that targets TRBC1 comprises a heavy chain comprising the amino acid sequence of SEQ ID NO: 6167 (or an amino acid sequence having at least about 93%, 95%, or 99% sequence identity to SEQ ID NO: 6167) and a light chain comprising the amino acid sequence of SEQ ID NO: 6169 (or an amino acid sequence having at least about 93%, 95%, or 99% sequence identity to SEQ ID NO: 6169). In some embodiments, the antigen binding domain that targets TRBC1 comprises a heavy chain comprising the amino acid sequence of SEQ ID NO: 6167 and a light chain comprising the amino acid sequence of SEQ ID NO: 6169.

In some embodiments, the antigen binding domain that targets TRBC1 comprises a heavy chain comprising the amino acid sequence of SEQ ID NO: 6168 (or an amino acid sequence having at least about 93%, 95%, or 99% sequence identity to SEQ ID NO: 6168) and a light chain comprising the amino acid sequence of SEQ ID NO: 6169 (or an amino acid sequence having at least about 93%, 95%, or 99% sequence identity to SEQ ID NO: 6169). In some embodiments, the antigen binding domain that targets TRBC1 comprises a heavy chain comprising the amino acid sequence of SEQ ID NO: 6168 and a light chain comprising the amino acid sequence of SEQ ID NO: 6169.

### TRBC2 Antigen Binding Domains

In some embodiments, the antigen binding domain that binds to TRBC2 comprises one or more CDRs (e.g., VHCDR1, VHCDR2, VHCDR3, VLCDR1, VLCDR2, and/or VLCDR3) disclosed in Table 9A, Table 9B, or Table 10, or a sequence having at least 85%, 90%, 95%, or 99% identity thereto. In some embodiments, the antigen binding domain that binds to TRBC2 comprises one or more framework regions (e.g., VHFWR1, VHFWR2, VHFWR3, VHFWR4, VLFWR1, VLFWR2, VLFWR3, and/or VLFWR4) disclosed in Table 9A, Table 9B, or Table 10, or a sequence having at least 85%, 90%, 95%, or 99% identity thereto. In some embodiments, the antigen binding domain that binds to TRBC2 comprises a VH and/or a VL disclosed in Table 11, or a sequence having at least 85%, 90%, 95%, or 99% identity thereto. In some embodiments, the antigen binding domain that binds to TRBC2 comprises an amino acid sequence disclosed in Table 12, or a sequence having at least 85%, 90%, 95%, or 99% identity thereto.

In some embodiments, the antigen binding domain that binds to TRBC2 comprises a VH comprising a heavy chain complementarity determining region 1 (VHCDR1), a VHCDR2, and a VHCDR3, and a VL comprising a light chain complementarity determining region 1 (VLCDR1), a VLCDR2, and a VLCDR3.

In some embodiments, the VHCDR1, VHCDR2, and VHCDR3 comprise the amino acid sequences of GX1X2MH, wherein X1 is Y or F, and X2 is P, H, V, Y, K, or A and SEQ ID NOs: 201, and 7442, respectively (or a sequence having at least 85%, 90%, 95%, or 99% identity thereto). In some embodiments, the VHCDR1, VHCDR2, and VHCDR3 comprise the amino acid sequences of SEQ ID NOs: 7422, 201, and 7403, respectively (or a sequence having at least 85%, 90%, 95%, or 99% identity thereto). In some embodiments, the VHCDR1, VHCDR2, and VHCDR3 comprise the amino acid sequences of SEQ ID NOs: 7401, 201, and 7403, respectively (or a sequence having at least 85%, 90%, 95%, or 99% identity thereto). In some embodiments, the VHCDR1, VHCDR2, and VHCDR3 comprise the amino acid sequences of SEQ ID NOs: 7394, 201, and 307, respectively (or a sequence having at least 85%, 90%, 95%, or 99% identity thereto). In some embodiments, the VHCDR1, VHCDR2, and VHCDR3 comprise the amino acid sequences of SEQ ID NOs: 8643, 201, and 8695, respectively (or a sequence having at least 85%, 90%, 95%, or 99% identity thereto). In some embodiments, the VHCDR1, VHCDR2, and VHCDR3 comprise the amino acid sequences of SEQ ID NOs: 8643, 201, and 7400, respectively (or a sequence having at least 85%, 90%, 95%, or 99% identity thereto). In some embodiments, the VHCDR1, VHCDR2, and VHCDR3 comprise the amino acid sequences of SEQ ID NOs: 7405, 201, and 7403, respectively (or a sequence having at least 85%, 90%, 95%, or 99% identity thereto). In some embodiments, the VHCDR1, VHCDR2, and VHCDR3 comprise the amino acid sequences of SEQ ID NOs: 7407, 201, and 7403, respectively (or a sequence having at least 85%, 90%, 95%, or 99% identity thereto). In some embodiments, the VHCDR1, VHCDR2, and VHCDR3 comprise the amino acid sequences of SEQ ID NOs: 7427, 201, and 7403, respectively (or a sequence having at least 85%, 90%, 95%, or 99% identity thereto). In some embodiments, the VHCDR1, VHCDR2, and VHCDR3 comprise the amino acid sequences of SEQ ID NOs: 7430, 201, and 7403, respectively (or a sequence having at least 85%, 90%, 95%, or 99% identity thereto).

In some embodiments, the VLCDR1, VLCDR2, and VLCDR3 comprise the amino acid sequences of SEQ ID NOs: 7443, 224, and 225 or 8680, respectively (or a sequence having at least 85%, 90%, 95%, or 99% identity thereto). In some embodiments, the VLCDR1, VLCDR2, and VLCDR3 comprise the amino acid sequences of SEQ ID NOs: 7410, 224, and 225 or 8680, respectively (or a sequence having at least 85%, 90%, 95%, or 99% identity thereto). In some embodiments, the VLCDR1, VLCDR2, and VLCDR3 comprise the amino acid sequences of SEQ ID NOs: 7409, 224, and 225 or 8680, respectively (or a sequence having at least 85%, 90%, 95%, or 99% identity thereto).

In some embodiments, the VHCDR1, VHCDR2, VHCDR3, VLCDR1, VLCDR2, and VLCDR3 comprise the amino acid sequences of GX1X2MH, wherein X1 is Y or F, and X2 is P, H, V, Y, K, or A and SEQ ID NOs: 201, 7442, 7443, 224, and 225 or 8680, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7422, 201, 7403, 7410, 224, and 225 or 8680, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7401, 201, 7403, 7410, 224, and 225 or 8680, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of: SEQ ID NOs: 7394, 201, 307, 7410, 224, and 225 or 8680, respectively (or a sequence having at least 85%, 90%, 95%, or 99% identity thereto); SEQ ID NOs: 8643, 201, 8695, 7410, 224, and 225 or 8680, respectively (or a sequence having at least 85%, 90%, 95%, or 99% identity thereto); SEQ ID NOs: 8643, 201, 7400, 7410, 224, and 225 or 8680, respectively (or a sequence having at least 85%, 90%, 95%, or 99% identity thereto); SEQ ID NOs: 7405, 201, 7403, 7410, 224, and 225 or 8680, respectively (or a sequence having at least 85%, 90%, 95%, or 99% identity thereto); SEQ ID NOs: 7407, 201, 7403, 7410, 224, and 225 or 8680, respectively (or a sequence having at least 85%, 90%, 95%, or 99% identity thereto); SEQ ID NOs: 7427, 201, 7403, 7410, 224, and 225 or 8680, respectively (or a sequence having at least 85%, 90%, 95%, or 99% identity thereto); SEQ ID NOs: 7430, 201, 7403, 7410, 224, and 225 or 8680, respectively (or a sequence having at least 85%, 90%, 95%, or 99% identity thereto); SEQ ID NOs: 7422, 201, 7403, 7409, 224, and 225 or 8680, respectively (or a sequence having at least 85%, 90%, 95%, or 99% identity thereto); SEQ ID NOs: 7401, 201, 7403, 7409, 224, and 225 or 8680, respectively (or a sequence having at least 85%, 90%, 95%, or 99% identity thereto); SEQ ID NOs: 7394, 201, 307, 7409, 224, and 225 or 8680, respectively (or a sequence having at least 85%, 90%, 95%, or 99% identity thereto); SEQ ID NOs: 8643, 201, 8695, 7409, 224, and 225 or 8680, respectively (or a sequence having at least 85%, 90%, 95%, or 99% identity thereto); SEQ ID NOs: 8643, 201, 7400, 7409, 224, and 225 or 8680, respectively (or a sequence having at least 85%, 90%, 95%, or 99% identity thereto); SEQ ID NOs: 7405, 201, 7403, 7409, 224, and 225 or 8680, respectively (or a sequence having at least 85%, 90%, 95%, or 99% identity thereto); SEQ ID NOs: 7407, 201, 7403, 7409, 224, and 225 or 8680, respectively (or a sequence having at least 85%, 90%, 95%, or 99% identity thereto); SEQ ID NOs: 7427, 201, 7403, 7409, 224, and 225 or 8680, respectively (or a sequence having at least 85%, 90%, 95%, or 99% identity thereto); or SEQ ID NOs: 7430, 201, 7403, 7409, 224, and 225 or 8680, respectively (or a sequence having at least 85%, 90%, 95%, or 99% identity thereto).

In some embodiments, the VH comprises an amino acid sequence selected from the group consisting of SEQ ID NOs: 7420, 7423, 7411, 7412, 7413, 7414, 7415, 7416, 7417, 7425, 7428, and 7431 (or a sequence having at least 85%, 90%, 95%, or 99% identity thereto) and/or the VL comprises an amino acid sequence selected from the group consisting of SEQ ID NOs: 7419 and 7418 (or a sequence having at least 85%, 90%, 95%, or 99% identity thereto). In some embodiments, the VH and VL comprise the amino acid sequences of SEQ ID NOs: 7420 and 7419, respectively (or a sequence having at least 85%, 90%, 95%, or 99% identity thereto). In some embodiments, the VH and VL comprise the amino acid sequences of SEQ ID NOs: 7423 and 7419, respectively (or a sequence having at least 85%, 90%, 95%, or 99% identity thereto). In some embodiments, the VH and VL comprise the amino acid sequences of SEQ ID NOs: 7411 and 7419, respectively (or a sequence having at least 85%, 90%, 95%, or 99% identity thereto). In some embodiments, the VH and VL comprise the amino acid sequences of SEQ ID NOs: 7412 and 7419, respectively (or a sequence having at least 85%, 90%, 95%, or 99% identity thereto). In some embodiments, the VH and VL comprise the amino acid sequences of SEQ ID NOs: 7413 and 7419, respectively (or a sequence having at least 85%, 90%, 95%, or 99% identity thereto). In some embodiments, the VH and VL comprise the amino acid sequences of SEQ ID NOs: 7414 and 7419, respectively (or a sequence having at least 85%, 90%, 95%, or 99% identity thereto). In some embodiments, the VH and VL comprise the amino acid sequences of SEQ ID NOs: 7415 and 7419, respectively (or a sequence having at least 85%, 90%, 95%, or 99% identity thereto). In some embodiments, the VH and VL comprise the amino acid sequences of SEQ ID NOs: 7416 and 7419, respectively (or a sequence having at least 85%, 90%, 95%, or 99% identity thereto). In some embodiments, the VH and VL comprise the amino acid sequences of SEQ ID NOs: 7417 and 7419, respectively (or a sequence having at least 85%, 90%, 95%, or 99% identity thereto). In some embodiments, the VH and VL comprise the amino acid sequences of SEQ ID NOs: 7425 and 7419, respectively (or a sequence having at least 85%, 90%, 95%, or 99% identity thereto). In some embodiments, the VH and VL comprise the amino acid sequences of SEQ ID NOs: 7428 and 7419, respectively (or a sequence having at least 85%, 90%, 95%, or 99% identity thereto). In some embodiments, the VH and VL comprise the amino acid sequences of SEQ ID NOs: 7431 and 7419, respectively (or a sequence having at least 85%, 90%, 95%, or 99% identity thereto). In some embodiments, the VH and VL comprise the amino acid sequences of SEQ ID NOs: 7420 and 7418, respectively (or a sequence having at least 85%, 90%, 95%, or 99% identity thereto). In some embodiments, the VH and VL comprise the amino acid sequences of SEQ ID NOs: 7423 and 7418, respectively (or a sequence having at least 85%, 90%, 95%, or 99% identity thereto). In some embodiments, the VH and VL comprise the amino acid sequences of SEQ ID NOs: 7411 and 7418, respectively (or a sequence having at least 85%, 90%, 95%, or 99% identity thereto). In some embodiments, the VH and VL comprise the amino acid sequences of SEQ ID NOs: 7412 and 7418, respectively (or a sequence having at least 85%, 90%, 95%, or 99% identity thereto). In some embodiments, the VH and VL comprise the amino acid sequences of SEQ ID NOs: 7413 and 7418, respectively (or a sequence having at least 85%, 90%, 95%, or 99% identity thereto). In some embodiments, the VH and VL comprise the amino acid sequences of SEQ ID NOs: 7414 and 7418, respectively (or a sequence having at least 85%, 90%, 95%, or 99% identity thereto). In some embodiments, the VH and VL comprise the amino acid sequences of SEQ ID NOs: 7415 and 7418, respectively (or a sequence having at least 85%, 90%, 95%, or 99% identity thereto). In some embodiments, the VH and VL comprise the amino acid sequences of SEQ ID NOs: 7416 and 7418, respectively (or a sequence having at least 85%, 90%, 95%, or 99% identity thereto). In some embodiments, the VH and VL comprise the amino acid sequences of SEQ ID NOs: 7417 and 7418, respectively (or a sequence having at least 85%, 90%, 95%, or 99% identity thereto). In some embodiments, the VH and VL comprise the amino acid sequences of SEQ ID NOs: 7425 and 7418, respectively (or a sequence having at least 85%, 90%, 95%, or 99% identity thereto). In some embodiments, the VH and VL comprise the amino acid sequences of SEQ ID NOs: 7428 and 7418, respectively (or a sequence having at least 85%, 90%, 95%, or 99% identity thereto). In some embodiments, the VH and VL comprise the amino acid sequences of SEQ ID NOs: 7431 and 7418, respectively (or a sequence having at least 85%, 90%, 95%, or 99% identity thereto).

In some embodiments, the antigen binding domain that binds to TRBC2 comprises an amino acid sequence selected from the group consisting of SEQ ID NOs: 7433, 7434, 7435, 7436, and 7437 (or a sequence having at least 85%, 90%, 95%, or 99% identity thereto).

In some embodiments, a bispecific antibody is contemplated herein to comprise a first antigen binding domain that binds to TRBC2. In some embodiments, an exemplary TRBC2 binding domain comprises a heavy chain and a light chain of a TRBC2 binding antibody (e.g., TRCBC2 binder-1, or TRCBC2 binder-2 or TRCBC2 binder 3, or TRCBC2 binder 4). In some embodiments, an exemplary TRBC2 binding domain comprises a heavy chain variable domain selected from SEQ ID NOs: 8011, 8013, 8020, or 8303, or a sequence that is at least 90% identical to SEQ ID NOs: 8011, 8013, 8020, or 8303; and a light chain variable domain selected from SEQ ID NOs: 8012, 8014, 8014, or 8014 or a sequence that is at least 90% identical to SEQ ID NOs: 1, 3, 31, or 33, including any combinations thereof.

In some embodiments, an exemplary TRBC2 binding domain comprises a heavy chain and a light chain of TRBC2 binding antibody e.g., TRCBC2 binder-1. In some embodiments, an exemplary TRBC2 binding domain comprises a sequence of amino acids in the heavy chain e.g., a heavy chain variable domain (VH) that is at least 90% identical to SEQ ID NO: 8011. In some embodiments, an exemplary TRBC2 binding domain comprises a heavy chain variable domain that is at least 91%, at least 92%, at least 93%, at least 94%, at least 95%, at least 96%, at least 97%, at least 98%, or at least 99% identical to SEQ ID NO: 8011. In some embodiments, the TRBC2 binding domain comprises a heavy chain comprising an amino acid sequence of SEQ ID NO: 8011.

In some embodiments, an exemplary TRBC2 binding domain comprises a sequence of amino acids in the light chain e.g., a light chain variable domain (VL) that is at least 95% identical to SEQ ID NO: 8012. In some embodiments, the light chain variable domain is at least at least 96%, at least 97%, at least 98%, or at least 99% identical to SEQ ID NO: 8012. In some embodiments, the TRBC2 binding domain comprises a light chain comprising an amino acid sequence of SEQ ID NO: 8012. In some embodiments, the bispecific antibody comprises a first antigen binding domain that binds to TRBC2 and comprises a heavy chain VH domain (SEQ ID NO: 8011) and a light chain VL domain (SEQ ID NO: 8012) of a TRBC2 binder, or a TRBC2 binder having one or more amino acid sequences described in Table 13 or Table 14, or a TRBC2 binder having any one of the domains described in Table 13 or Table 14, or one or more domains with one or more amino acid modifications relative to the amino acid sequences of domains described in Table 13 or Table 14.

In some embodiments, an exemplary TRBC2 binding domain comprises a heavy chain and a light chain of TRBC2 binding antibody e.g., TRCBC2 binder-2. In some embodiments, an exemplary TRBC2 binding domain comprises a heavy chain that is at least 95% identical to SEQ ID NO:8013. In some embodiments, an exemplary TRBC2 binding domain comprises a heavy chain that is at least 96%, at least 97%, at least 98%, or at least 99% identical to SEQ ID NO: 8013. In some embodiments, an exemplary TRBC2 binding domain comprises a light chain that is at least 95% identical to SEQ ID NO: 8014. In some embodiments, an exemplary TRBC2 binding domain comprises a light chain that is at least at least 96%, at least 97%, at least 98%, or at least 99% identical to SEQ ID NO: 8014. In some embodiments, the bispecific antibody comprises a first antigen binding domain that binds to TRBC2 that comprises a heavy chain VH (SEQ ID NO: 8013) and a light chain VL (SEQ ID NO: 8014) of a TRBC2 binder, or a TRBC2 binder having any one of the domains described in Table 13, or one or more domains with one or more amino acid modifications relative to the amino acid sequences of domains described in Table 13.

In some embodiments, an exemplary TRBC2 binding domain comprises a sequence of amino acids in the heavy chain e.g., a heavy chain variable domain (VH) that is at least 90% identical to SEQ ID NO: 8020. In some embodiments, an exemplary TRBC2 binding domain comprises a heavy chain variable domain that is at least 91%, at least 92%, at least 93%, at least 94%, at least 95%, at least 96%, at least 97%, at least 98%, or at least 99% identical to SEQ ID NO: 8020.

In some embodiments, an exemplary TRBC2 binding domain comprises a sequence of amino acids in the light chain e.g., a light chain variable domain (VL) that is at least 90% identical to SEQ ID NO: 8014. In some embodiments, an exemplary TRBC2 binding domain comprises a light chain variable domain that is at least 91%, at least 92%, at least 93%, at least 94%, at least 95%, at least 96%, at least 97%, at least 98%, or at least 99% identical to SEQ ID NO: 8014. In some embodiments, the bispecific antibody comprises a first antigen binding domain that binds to TRBC2 and comprises a heavy chain VH (SEQ ID NO: 8020) and a light chain VL (SEQ ID NO: 8014) of a TRBC2 binder, or a TRBC2 binder having any one of the domains described in Table 13, or one or more domains with one or more amino acid modifications relative to the amino acid sequences of domains described in Table 13 or Table 14.

In some embodiments, an exemplary TRBC2 binding domain comprises a sequence of amino acids in the heavy chain e.g., a heavy chain variable domain (VH) that is at least 90% identical to SEQ ID NO: 8303. In some embodiments, an exemplary TRBC2 binding domain comprises a heavy chain variable domain that is at least 91%, at least 92%, at least 93%, at least 94%, at least 95%, at least 96%, at least 97%, at least 98%, or at least 99% identical to SEQ ID NO: 8303.

In some embodiments, an exemplary TRBC2 binding domain comprises a sequence of amino acids in the light chain e.g., a light chain variable domain (VL) that is at least 90% identical to SEQ ID NO: 8014. In some embodiments, the bispecific antibody comprises a first antigen binding domain that binds to TRBC2 and comprises a heavy chain VH (SEQ ID NO: 8013) and a light chain VL (SEQ ID NO: 8024) of a TRBC2 binder, or a TRBC2 binder having any one of the domains described in Table 13, or one or more domains with one or more amino acid modifications relative to the amino acid sequences of domains described in Table 13.

For example, a TRBC2-binding Heavy Chain domain may comprise the sequence of SEQ ID NO:8011 QVQLVQSGAEVKKPGSSVKVSCKASPRGFYGYHMHWVRQAPGQGLEWMGFINPYTNDIQYNE RFRGRVTITSDESTTTAYMELSSLRSEDTAVYYCAMGNGKWGDGAYRFFDLWGQGTLVTVSS (SEQ ID NO: 8011), and a TRBC2-binding Light Chain domain may comprise the sequence of SEQ ID NO:8012

(SEQ ID NO: 8012)
DVVMTQSPLSLPVTLGQPASISCRSSENLVHSNGRTYLQWYQQRPGQ
SPRLLIYRVSNRFPGVPDRFSGSGSGTDFTLKISRVEAEDVGVYFCS
QSSLEPYTFGGGTKVEIK.

For example, a TRBC2-binding Heavy Chain domain may comprise the sequence of SEQ ID NO:8013 QVQLVQSGAEVKKPGSSVKVSCKASPRGFYGYHMHWVRQAPGQGLEWMGFINPYNNHIQYNE RFRGRVTITSDESTTTAYMELSSLRSEDTAVYYCALGNGKWGDGAYRFFDFWGQGTLVTVSS (SEQ ID NO: 8013), and a TRBC2-binding Light Chain domain may comprise the sequence of SEQ ID NO: 8014

(SEQ ID NO: 8014)
DVVMTQSPLSLPVTLGQPASISCRSSKNLVHSNGRTYLQWYQQRPGQSPR
LLIYRVSNRFPGVPDRFSGSGSGTDFTLKISRVEAEDVGVYFCSQSTREP
YTFGGGTKVEIK

A TRBC2-binding Heavy Chain domain may comprise the sequence of SEQ ID NO:8020 QVQLVQSGAEVKKPGSSVKVSCKASPRGFYGYHMHWVRQAPGQGLEWMGFINPYNNHIQYNE RFRGRVTITSDESTTTAYMELSSLRSEDTAVYYCALGEGKWGDGAYRFFDFWGQGTLVTVSS (SEQ ID NO: 8020), and a TRBC2-binding Light Chain domain may comprise the sequence of SEQ ID NO: 8014:

(SEQ ID NO: 8014)
DVVMTQSPLSLPVTLGQPASISCRSSKNLVHSNGRTYLQWYQQRPGQSPR
LLIYRVSNRFPGVPDRFSGSGSGTDFTLKISRVEAEDVGVYFCSQSTREP
YTFGGGTKVEIK.

A TRBC2-binding Heavy Chain domain may comprise the sequence of SEQ ID NO:8303 QVQLVQSGAEVKKPGSSVKVSCKASPRGFYGYHMHWVRQAPGQGLEWMGFINPYNNHIQYNE RFRGRVTITSDESTTTAYMELSSLRSEDTAVYYCALGAGKWGDGAYRFFDFWGQGTLVTVSS (SEQ ID NO: 8303), and a TRBC2-binding Light Chain domain may comprise the sequence of SEQ ID NO: 8014:

(SEQ ID NO: 8014)
DVVMTQSPLSLPVTLGQPASISCRSSKNLVHSNGRTYLQWYQQRPGQSPR
LLIYRVSNRFPGVPDRFSGSGSGTDFTLKISRVEAEDVGVYFCSQSTREP
YTFGGGTKVEIK.

In some embodiments, the TRBC2 antigen binding domain may comprise a mutation in the heavy chain variable domain, the mutation may be selected from a T28K mutation, a Y32F mutation and an A100N mutation in the VH domain compared to a VH domain of an antibody as described in SEQ ID NO: 8024 In some embodiments, the TRBC2 antigen binding domain comprises two or more mutations in the heavy chain variable domain, the mutation may be selected from a T28K mutation, a Y32F mutation and an A100N mutation in the VH domain compared to VH domain of the antibody as described in SEQ ID NO: 8024. In some embodiments, the VH domain may comprise a mutation that may be at position V2, or Y27, or G31 or R98, or Y102, or N103 or A107 with respect to SEQ ID NO: 8024. In some embodiments, the VH domain of the TRBC2 antigen binding domain may comprise a V2K or a V2R mutation compared to VH domain of the antibody as described in SEQ ID NO: 8024. In some embodiments, the VH domain of the TRBC2 antigen binding domain may comprise a Y27F, Y27M, Y27N or Y27W mutation compared to VH domain of the antibody as described in SEQ ID NO: 8024. In some embodiments, the VH domain of the TRBC2 antigen binding domain may comprise a G31K, a G31R, or a G31S mutation compared to VH domain of the antibody as described in SEQ ID NO: 8024. In some embodiments, the VH domain of the TRBC2 antigen binding domain may comprise a R98K mutation compared to VH domain of the antibody as described in SEQ ID NO: 8024. In some embodiments, the VH domain of the TRBC2 antigen binding domain may comprise a Y102F or Y102L mutation compared to VH domain of the antibody as described in SEQ ID NO: 8024. In some embodiments, the VH domain of the TRBC2 antigen binding domain may comprise a N193A, N193E, N103F, N103H, N103L, N103M, N103Q, N103S, N103W, or N103Y mutation compared to VH domain of the antibody as described in SEQ ID NO: 8024.

In some embodiments, the TRBC2 antigen binding domain may comprise a mutation in the light chain variable domain, the mutation may be at position N35, or at R55 with respect to the VL domain of an antibody as described in SEQ ID NO: 8025. In some embodiments, the VL domain of the TRBC2 antigen binding domain may comprise a N35M, N35F, N35Y, N35K or N35R mutation at position 35 with respect to the VL domain of an antibody as described in SEQ ID NO: 8025.

SEQ ID NO: 8024-Reference antibody VH domain:
(SEQ ID NO: 8024)
QVQLVQSGAEVKKPGASVKVSCKASGYTFTGYVMHWVRQAPGQGLEWMGF
INPYNDDIQSNERFRGRVTMTRDTSISTAYMELSRLRSDDTAVYYCARGA
GYNFDGAYRFFDFWGQGTMVTVSS.
SEQ ID NO: 8025-Reference antibody VL domain:
(SEQ ID NO: 8025)
DIVMTQSPLSLPVTPGEPASISCRSSQRLVHSNGNTYLHWYLQKPGQSPR
LLIYRVSNRFPGVPDRFSGSGSGTDFTLKISRVEAEDVGVYYCSQSTHVP
YTFGQGTKLEIK

In some embodiments, the bispecific antibody comprises a second antigen binding domain that binds to NKp30. In some embodiments, an exemplary NKp30 binding domain comprises a heavy chain and a light chain of NKp30 binder 1. In some embodiments, an exemplary NKp30 binding domain comprises a heavy chain having an amino acid sequence of SEQ ID NO: 8006 or 8698. In some embodiments, an exemplary NKp30 binding domain comprises a heavy chain having an amino acid sequence that is at least 90% identical to a sequence of SEQ ID NO: 8006 or 8698. In some embodiments, an exemplary NKp30 binding domain comprises a heavy chain having an amino acid sequence that is at least 91%, at least 92%, at least 93%, at least 94%, at least 95%, at least 96%, at least 97%, at least 98%, or at least 99% identical to a sequence of SEQ ID NO: 8006 or 8698.

In some embodiments, an exemplary NKp30 binding domain comprises a heavy chain and a light chain of NKp30 binder 1. In some embodiments, an exemplary NKp30 binding domain comprises a heavy chain having an amino acid sequence of SEQ ID NO: 4684 or 8690. In some embodiments, an exemplary NKp30 binding domain comprises a heavy chain having an amino acid sequence that is at least 90% identical to a sequence of SEQ ID NO: 4684 or 8690. In some embodiments, an exemplary NKp30 binding domain comprises a heavy chain having an amino acid sequence that is at least 91%, at least 92%, at least 93%, at least 94%, at least 95%, at least 96%, at least 97%, at least 98%, or at least 99% identical to a sequence of SEQ ID NO: 4684 or 8690.

In some embodiments, an exemplary NKp30 binding domain comprises a light chain of SEQ ID NO: 8003. In some embodiments, an exemplary NKp30 binding domain comprises a light chain of SEQ ID NO: 4683. In some embodiments, an exemplary NKp30 binding domain comprises a light chain having an amino acid sequence that is at least 90% identical to a sequence of SEQ ID NO: 8003. In some embodiments, an exemplary NKp30 binding domain comprises a light chain having an amino acid sequence that is at least 90% identical to a sequence of SEQ ID NO: 4683. In some embodiments, an exemplary NKp30 binding domain comprises a light chain having an amino acid sequence that is at least 91%, at least 92%, at least 93%, at least 94%, at least 95%, at least 96%, at least 97%, at least 98%, or at least 99% identical to a sequence of SEQ ID NO: 8003. In some embodiments, an exemplary NKp30 binding domain comprises a light chain having an amino acid sequence that is at least 91%, at least 92%, at least 93%, at least 94%, at least 95%, at least 96%, at least 97%, at least 98%, or at least 99% identical to a sequence of SEQ ID NO: 4683. In some embodiments, the bispecific antibody comprising a second antigen binding domain that binds to NKp30 comprises a heavy chain and a light chain of NKp30 binder 1, having amino acid sequences described in below.

An exemplary NKp30-binding, Heavy Chain domain may comprise the sequence of SEQ ID NO: 8030:

(SEQ ID NO: 8030)
EIQLLESGGGLVQPGGSLRLSCAVSGFSITTTGYHWNWVRQAPGKGLEWV
GYIYSSGSTSYNPSLKSRFTISRDTSKNTFYLQMNSLRAEDTAVYYCARG
DWHYFDYWGQGTMVTVSS.

An exemplary NKp30-binding, Light Chain domain may comprise the sequence of SEQ ID NO: 13:

(SEQ ID NO: 13)
DSVTTQSPLSLPVTLGQPASISCSGEKLSDKYVHWYQQRPGQSPRMLIYE
NDRRPSGVPDRFSGSNSGNDATLKISRVEAEDVGVYFCQFWDSTNSAVFG
GGTKVEIK

In some embodiments, an exemplary bispecific antibody comprises (i) a first antigen binding domain comprising a TRBC2 binding domain having a heavy chain sequence of SEQ ID NO: 8011, or a sequence that is at least 90% identical to SEQ ID NO: 8011; and a light chain sequence of SEQ ID NO: 8012, or a sequence that is at least 95% identical to SEQ ID NO: 8012; and (ii) a second antigen binding domain comprising a NKp30 binding domain having a heavy chain and a light chain sequence of SEQ ID NOs: 8030 and 13 respectively.

In some embodiments, an exemplary bispecific antibody comprises (i) a first antigen binding domain comprising a TRBC2 binding domain having a heavy chain sequence of SEQ ID NO: 8013, or a sequence that is at least 95% identical to SEQ ID NO: 8013; and a light chain sequence of SEQ ID NO: 8014, or a sequence that is at least 95% identical to SEQ ID NO: 8014; and (ii) a second antigen binding domain comprising a NKp30 binding domain having a heavy chain and a light chain sequence of SEQ ID NO: 8030 and 13 respectively.

In some embodiments, an exemplary bispecific antibody comprises (i) a first antigen binding domain comprising a TRBC2 binding domain having a heavy chain sequence of SEQ ID NO: 8011, or a sequence that is at least 90% identical to SEQ ID NO: 8011 and a light chain sequence of SEQ ID NO: 8012, or a sequence that is at least 95% identical to SEQ ID NO: 8012; and (ii) a second antigen binding domain comprising a NKp30 binding domain having a heavy chain and a light chain sequence at least 90% identical to SEQ ID NO: 8030 and at least 90% identical to SEQ ID NO: 13 respectively.

In some embodiments, an exemplary bispecific antibody comprises (i) a first antigen binding domain comprising a TRBC2 binding domain having a heavy chain sequence of SEQ ID NO: 8013, or a sequence that is at least 95% identical to SEQ ID NO: 8013 and a light chain sequence of SEQ ID NO: 8014, or a sequence that is at least 95% identical to SEQ ID NO: 8014; and (ii) a second antigen binding domain comprising a NKp30 binding domain having a heavy chain and a light chain sequence at least 90% identical to SEQ ID NO: 8030 and at least 90% identical to SEQ ID NO: 13 respectively.

In some embodiments, the bi-specific antibody comprising: (i) a first antigen binding domain comprising a TRBC2 binding domain that comprises a single chain variable fragment (scFv); wherein the scFv comprises a heavy chain and a light chain that are linked by a short linker, and (ii) a second antigen binding domain comprising a NKp30 binding domain that comprises a single chain variable fragment (scFv); wherein scFv comprises a heavy chain and a light chain that are linked by a short linker.

In some embodiments, the heavy chain of a TRBC2 binding domain comprises a HC CDR1, a CDR2 and a CDR3. In some embodiments, the light chain of a TRBC2 binding domain comprises a LC CDR1, a CDR2 and a CDR3. In one embodiment, the heavy chain of a TRBC2 binding domain comprises a HC CDR1 having an amino acid sequence comprising PRGFYGYHMH (SEQ ID NO: 8272) {or PRGFYGY (SEQ ID NO: 8041 according to Chothia numbering system)}, or PRGFYGYAMH (SEQ ID NO: 8701). In one embodiment, the heavy chain of a TRBC2 binding domain comprises a HC CDR1 having an amino acid sequence RSGFHGYAMH (SEQ ID NO: 8207) or PRGFHGYHMH (SEQ ID NO: 8211). In some embodiments, the heavy chain TRBC2 binding domain comprises a HC CDR1 comprising an amino acid sequence SRSGFHGYAMH (SEQ ID NO: 8215). In some embodiments, the heavy chain TRBC2 binding domain comprises a HC CDR1 comprising an amino acid sequence PRGFYGYHMH (SEQ ID NO: 8272). In some embodiments, the heavy chain TRBC2 binding domain comprises a HC CDR1 comprising an amino acid sequence RSSQNLVHSNGRTYLH (SEQ ID NO: 8226). In one embodiment, the heavy chain of a TRBC2 binding domain comprises a HC CDR2 having an amino acid sequence MGFINPYTNDIQYNERFRG (SEQ ID NO: 8042). In one embodiment, the heavy chain of a TRBC2 binding domain comprises a HC CDR3 having an amino acid sequence GNGKWGDGAYRFFDL (SEQ ID NO: 8043). In one embodiment, the heavy chain of a TRBC2 binding domain comprises a HC CDR2 comprising an amino acid sequence FINPYNNIHIQYNERFRC (SEQ ID NO: 8044) or FINPYNDDIQYNQKFQG (SEQ ID NO: 8208) or YINPYNRDIKYNQKFQG (SEQ ID NO: 8212). In some embodiments, the HC CDR2 has an anino acid sequence FINPYNHAIKYNQKFQG (SEQ ID NO: 8213). In some embodiments, the HC CDR2 has an amino acid sequence YINPYTGDIKYNERFRG (SEQ ID NO: 8217). In some embodiments, the HC CDR2 has an amino acid sequence FINPYNDDIQYNERFRG (SEQ ID NO. 8221). In some embodiments, the HC CDR2 has an amino acid sequence TINPYNAEIKYNQKFQG (SEQ ID NO: 8222). In some embodiments, the HC CDR2 has an amino acid sequence TINPYNRDIQYNQKFQG (SEQ ID NO: 8225). In some embodiments, the HC CDR2 has an amino acid sequence FINPYNRDIKYNERFRG (SEQ ID NO: 8228). In some embodiments, the HC CDR2 has an amino acid sequence AINPYTNDIKYNERFRG (SEQ ID NO: 8229). In some embodiments, the HC CDR2 has an amino acid sequence AINPYTNHIQYNERFRG (SEQ ID NO: 8230). In some embodiments, the HC CDR2 has an amino acid sequence AINPYTRAIKYNERFRG (SEQ ID NO: 8231). In some embodiments, the HC CDR2 has an amino acid sequence TINPYNGDIQYNERFRG (SEQ ID NO: 8232). In some embodiments, the HC CDR2 has an amino acid sequence AINPYNTDIKYNERFRG (SEQ ID NO: 8233). In some embodiments, the HC CDR2 has an amino acid sequence YINPYNGAIKYNQKFQG (SEQ ID NO: 8234). In some embodiments, the HC CDR2 has an amino acid sequence AINPYNDDIQSNERFRG (SEQ ID NO: 8235). In some embodiments, the HC CDR2 has an amino acid sequence FINPYNRAIQYNQKFQG (SEQ ID NO: 8236). In some embodiments, the HC CDR2 has an amino acid sequence FINPYTNEIQYNERFRG (SEQ ID NO: 8237). In sonme embodiments, the HC CDR2 has an amino acid sequence YINPYNHDIQYNQKFQG (SEQ ID NO: 8700). In some embodiments, the HC CDR2 has an amnio acid sequence SINPYTHDIQYNERFRG (SEQ ID NO: 8238). In some embodiments, the HC CDR2 has an amino acid sequence YINPYKNAIQYNQKFQG (SEQ ID NO: 8239). In some embodiments, the HC CDR2 has an amino acid sequence AINPYNTDIQYNERFRG (SEQ ID NO: 8240). In some embodiments, the HC CDR2 has an amino acid sequence SINPYNGDIQYNERFRG (SEQ ID NO: 8241). In some embodiments, the HC CDR2 has an amino acid sequence TINPYNHDAQYNERFRG (SEQ ID NO: 8242). In some embodiments, the HC CDR2 has an amino acid sequence AINPYNDDIKYNERFRG (SEQ ID NO: 8243). In some embodiments, the HC CDR2 has an amino acid sequence YINPYTHEIKYNERFRG (SEQ ID NO: 8245). In some embodiments, the HC CDR2 has an amino acid sequence FINPYKDDIKYNERFRG (SEQ ID NO: 8246). In some embodiments, the HC CDR2 has an amino acid sequence AINPYNDDIKYNQKFQG (SEQ ID NO: 8247). In some embodiments, the HC CDR2 has an amino acid sequence AINPYNRDIKYNERFRG (SEQ ID NO: 8248). In some embodiments, the HC CDR2 has an amino acid sequence AINPYNGDIKYNERFRG (SEQ ID NO: 8249). In some embodiments, the HC CDR2 has an amino acid sequence YINPYTRDIKYNERFRG (SEQ ID NO: 8250). In some embodiments, the HC CDR2 has an amino acid sequence TINPYNTDIKYNERFRG (SEQ ID NO: 8251). In some embodiments, the HC CDR2 has an amino acid sequence TINPYNNDIQYNERFRG (SEQ ID NO: 8252). In some embodiments, the HC CDR2 has an anino acid sequence YINPYNGNIQYNERFRG (SEQ ID NO: 8253). In some embodiments, the HC CDR2 has an amino acid sequence AINPYTNEIQYNERFRG (SEQ ID NO: 8254). In some embodiments, the HC CDR2 has an amino acid sequence SINPYNHDIKYNERFRG (SEQ ID NO: 8255). In some embodiments, the HC CDR2 has an amino acid sequence FINPYKNEIKYNERFRG (SEQ ID NO: 8256). In some embodiments, the HC CDR2 has an amino acid sequence YINPYNNEIQYNERFRGR (SEQ ID NO: 8257) or a sequence SJNPYNRHIQYNERFRG (SEQ ID NO: 8258) or a sequence SINPYTREIQYNERFRG (SEQ ID NO: 8259). In some embodiments, the HC CDR2 has an amino acid sequence FINPYTNDIQYNERFRG (SEQ ID NO: 8260). In some embodiments, the HC CDR2 has an amino acid sequence AINPYTNEIKYNERFRG (SEQ ID NO: 8261). In some embodiments, the HC CDR2 has an amino acid sequence AINPYNDDIQYNERFRG (SEQ ID NO: 8263). In some embodiments, the HC CDR2 has an amino acid sequence YINPYNNDIKYNQKFQG (SEQ ID NO: 8264) or an amino acid sequence TINPYTREIQYNQKFQG (SEQ ID NO: 8266) or YINPYNNEIQYNQKFQG (SEQ ID NO: 8267). In some embodiments, the HC CDR2 has an amino acid sequence AINPYNHEIQYNQKFQG (SEQ ID NO: 8268). In some embodiments, the LC CDR2 has an amino acid sequence TINPYKHHIKYNERFRG (SEQ ID NO: 8269). In some embodiments, the HC CDR2 has an amino acid sequence FINPYTRAIKYNERFRG (SEQ ID NO: 8270). In some embodiments, the HC CDR2 has an amino acid sequence SINPYTRHIQYNERFRG (SEQ ID NO: 8273).

In one embodiment, the heavy chain of a TRBC2 binding domain comprises a HC CDR3 having an amino acid sequence GNGKWGDGAYRFFDF (SEQ ID NO: 7403). In some embodiments the heavy chain of a TRBC2 binding domain comprises a HC CDR3 having an amino acid sequence GEGKWGDGAYRFFDF (SEQ ID NO: 8046) In some embodiments the heavy chain of a TRBC2 binding domain comprises a HC CDR3 having an amino acid sequence GAGKWGDGAYRFFDF (SEQ ID NO: 8047). In some embodiments the heavy chain of a TRBC2 binding domain comprises a HC CDR3 having an amino acid sequence GNGKWGDGAYRFFDF (SEQ ID NO. 7403). In some embodiments the heavy chain of a TRBC2 binding domain comprises a HC CDR3 having an amino acid sequence GNGKWGDGAYRFFDL (SEQ ID NO. 8227). In some embodiments the heavy chain of a TRBC2 binding domain comprises a HC CDR3 having an amino acid sequence LGNGKWGDGAYRFFDL (SEQ ID NO: 8224). In some embodiments the heavy chain of a TRBC2 binding domain comprises a HC CDR3 having an amino acid sequence MGNGKWGDGAYRFFDL (SEQ ID NO: 8220). In some embodiments the heavy chain of a TRBC2 binding domain comprises a HC CDR3 having an amino acid sequence GNGKWGDGAYRFFDF (SEQ ID NO: 7403).

In one embodiment, the light chain of a TRBC2 binding domain comprises a LC CDR1 having an amino acid sequence RSSENLVHSNGRTYLQ (SEQ ID NO: 8048) or RSSQNLVHSNGRTYLQ (SEQ ID NO: 7410). In one embodiment, the light chain of a TRBC2 binding domain comprises a LC CDR1 having an amino acid sequence RSSQNLVHSNARTYLQ (SEQ ID NO: 8276). In one embodiment, the light chain of a TRBC2 binding domain comprises a LC CDR2 having an amino acid sequence RVSNRFP (SEQ ID NO: 224).

In one embodiment, the light chain of a TRBC2 binding domain comprises a LC CDR3 having an amino acid sequence SQSSLEPYT (SEQ ID NO: 8050) or SQSSYVPFT (SEQ ID NO: 8214). In one embodiment, the light chain of a TRBC2 binding domain comprises a LC CDR3 having an amino acid sequence SQSTYEPFT (SEQ ID NO: 8223). In one embodiment, the light chain of a TRBC2 binding domain comprises a LC CDR1 having an amino acid sequence RSSKNLVHSNGRTYLQ (SEQ ID NO: 8051) In one embodiment, the light chain of a TRBC2 binding domain comprises a LC CDR1 having an amino acid sequence RSSKNLVHSNARTYLQ (SEQ ID NO: 8271). In one embodiment, the light chain of a TRBC2 binding domain comprises a LC CDR3 having an amino acid sequence SQSTREPYT (SEQ ID NO: 8052) or SQSTHVPYT (SEQ ID NO: 225). In one embodiment, the light chain of a TRBC2 binding domain comprises a LC CDR3 having an amino acid sequence SQSTHLPYT (SEQ ID NO: 8262). In one embodiment, the light chain of a TRBC2 binding domain comprises a LC CDR3 having an amino acid sequence SQSTQEPYT (SEQ ID NO: 8265). In one embodiment, the light chain of a TRBC2 binding domain comprises a LC CDR3 having an amino acid sequence SQSSLLPYTF (SEQ ID NO: 8274). In one embodiment, the light chain of a TRBC2 binding domain comprises a LC CDR3 having an amino acid sequence SQSTLEPFT (SEQ ID NO: 8277). In one embodiment, the light chain of a TRBC2 binding domain comprises a LC CDR3 having an amino acid sequence SQSSHIPYT (SEQ ID NO: 8279). In one embodiment, the light chain of a TRBC2 binding domain comprises a LC CDR3 having an amino acid sequence SQSSLVPYT (SEQ ID NO: 8280).

In some embodiments, the heavy chain of a NKp30 binding domain comprises a HC CDR1, a CDR2 and a CDR3. In some embodiments, the light chain of a NKp30 binding domain comprises a LC CDR1, a CDR2 and a CDR3. In some embodiments, the heavy chain of a NKp30 binding domain comprises a HC CDR1, having an amino acid sequence ITTTGYHWN (SEQ ID NO: 308) or comprises a sequence GYHWN (SEQ ID NO:6000, according to Kabat numbering system). In some embodiments, the heavy chain of a NKp30 binding domain comprises a HC CDR2, having an amino acid sequence YIYSSGSTSYNPSLKS (SEQ ID NO: 6001). In some embodiments, the heavy chain of a NKp30 binding domain comprises a HC CDR3, having an amino acid sequence GDWHYFDY (SEQ ID NO: 504).

In some embodiments, the light chain of a NKp30 binding domain comprises a LC CDR1, having an amino acid sequence SGEKLSDKYVH (SEQ ID NO: 26). In some embodiments, the light chain of a NKp30 binding domain comprises a LC CDR2, having an amino acid sequence ENDRRPS (SEQ ID NO: 28). In some embodiments, the light chain of a NKp30 binding domain comprises a LC CDR3, having an amino acid sequence QFWDSTNSAV (SEQ ID NO: 86).

Additional TRBC2 binding variable heavy chain (VH) and variable light chain (VL) sequences that are used for generating anti-TRBC2 scFv are listed in Table 15.

In some embodiments, the bispecific antibody is a humanized antibody.

In some embodiments, the first antigen binding domain has a higher affinity for a T cell receptor comprising TRBC2 than for a T cell receptor not comprising TRBC2, optionally wherein the KD for the binding between the first antigen binding domain and TRBC2 is no more than 40%, 30%, 20%, 10%, 1%, 0.1%, or 0.01% of the KD for the binding between the first antigen binding domain and a T cell receptor not comprising TRBC2. In some embodiments, the first antigen binding domain has a higher affinity for a T cell receptor comprising TRBC2 than for a T cell receptor comprising TCRBC1, optionally wherein the K_{D} for the binding between the first antigen binding domain and TRBC2 is no more than 40%, 30%, 20%, 10%, 1%, 0.1%, or 0.01% of the K_{D} for the binding between the first antigen binding domain and a T cell receptor comprising TCRBC1. In some embodiments, binding of the first antigen binding domain to TRBC2 on a lymphoma cell or lymphocyte, e.g., T cell, does not appreciably activate the lymphoma cell or lymphocyte, e.g., T cell, e.g., as measured by T cell proliferation, expression of a T cell activation marker (e.g., CD69 or CD25), and/or expression of a cytokine (e.g., TNFα and IFNγ). In some embodiments, the multifunctional molecule does not activate NK cells or does not substantially activate NK cells in the absence of a TRBC2-expressing cell.

In some embodiments, the antigen-binding domain may comprise a mutation or a combination of the following mutations:
T28K, Y32F, A100N, Y27N in the VH domain,
T28K, Y32F, A100N, G31K in the VH domain,
T28K, Y32F, A100N, Y27M in the VH domain,
T28K, Y32F, A100N, Y27W in the VH domain,
T28K, Y32F, A100N in the VH domain,
T28K, Y32F, A100N in the VH domain and R55K in the VL domain,
T28K, Y32F, A100N in the VH domain and N35K in the VL domain,
T28K, Y32F, A100N, N103H in the VH domain,
T28K, Y32F, A100N, N103A in the VH domain,
T28K, Y32F, A100N, N103Y in the VH domain,
T28K, Y32F, A100N in the VH domain and N35R in the VL domain,
T28K, Y32F, A100N, N103S in the VH domain and N35M in the VL domain,
T28K, Y32F, A100N, N103M, in the VH domain,
T28K, Y32F, A100N, N103W in the VH domain and N35R in the VL domain,
T28K, Y32F, A100N in the VH domain and N35F in the VL domain,
T28K, Y32F, A100N, N103S in the VH domain and N35K in the VL domain,
T28K, Y32F, A100N, R98K in the VH domain,
T28K, Y32F, A100N, N103S in the VH domain and N35R in the VL domain,
T28K, Y32F, A100N, N103L in the VH domain,
T28K, Y32F, A100N, N103S in the VH domain and N35F in the VL domain,
T28K, Y32F, A100N, N103S in the VH domain and N35Y in the VL domain,
T28K, Y32F, A100N, N103L in the VH domain and N35M in the VL domain,
T28K, Y32F, A100N, N103L in the VH domain and N35R in the VL domain,
T28K, Y32F, A100N, N103W in the VH domain and N35K in the VL domain,
T28K, Y32F, A100N, N103L in the VH domain and N35Y in the VL domain,
T28K, Y32F, A100N, N103F in the VH domain,
T28K, Y32F, A100N, N103W in the VH domain,
T28K, Y32F, A100N, N103L in the VH domain and N35K in the VL domain,
T28K, Y32F, A100N, N103L in the VH domain and N35F in the VL domain,
T28K, Y32F, A100N, N103W in the VH domain and N35M in the VL domain,
T28K, Y32F, A100N, N103F in the VH domain and N35Y in the VL domain,
T28K, Y32F, A100N, Y27F in the VH domain,
T28K, Y32F, A100N, N103Q in the VH domain,
T28K, Y32F, A100N, N103S in the VH domain,
T28K, Y32F, A100N, N103M in the VH domain and N35F in the VL domain,
T28K, Y32F, A100N, N103F in the VH domain and N35M in the VL domain,
T28K, Y32F, A100N, N103F in the VH domain and N35F in the VL domain,
T28K, Y32F, A100N, G31R in the VH domain,
T28K, Y32F, A100N, N103W in the VH domain and N35F in the VL domain,
T28K, Y32F, A100N, V2R in the VH domain,
T28K, Y32F, A100N, G31S in the VH domain,
T28K, Y32F, A100N, A107S in the VH domain,
T28K, Y32F, A100N, N103E in the VH domain and N35M in the VL domain,
T28K, Y32F, A100N, V2K in the VH domain,
T28K, Y32F, A100N, N103E in the VH domain,
T28K, Y32F, A100N, Y102F, N103M in the VH domain and N35K in the VL domain,
T28K, Y32F, A100N, Y102F, N103M in the VH domain and N35F in the VL domain,
T28K, Y32F, A100N, Y102F, N103M in the VH domain and N35R in the VL domain,
T28K, Y32F, A100N, Y102F in the VH domain and N35R in the VL domain,
T28K, Y32F, A100N, N103M in the VH domain and N35M in the VL domain,
T28K, Y32F, A100N, N103M in the VH domain and N35Y in the VL domain,
T28K, Y32F, A100N, N103M in the VH domain and N35R in the VL domain,
T28K, Y32F, A100N, N103F in the VH domain and N35K in the VL domain,
T28K, Y32F, A100N, Y102L, N103W in the VH domain and N35R in the VL domain,
T28K, Y32F, A100N, Y102L, N103W in the VH domain and N35K in the VL domain,
T28K, Y32F, A100N, Y102F in the VH domain, and
T28K, Y32F, A100N, Y102L, N103M in the VH domain and N35R in the VL domain.

The residues describing the mutations as list above are considered with respect to a TCRBC1 or a TRBC2 wild type sequence. In some embodiments, the TCRBC1 or a TRBC2 wild type sequences or reference sequences. In some embodiments, the reference VH and VL sequences as depicted in SEQ ID NO: 8024 and SEQ ID NO:8025 respectively.

In some embodiments, the bispecific antibody comprises: (i) a first antigen binding domain comprising an scFv that binds to TRBC2 domain, and may comprise a heavy chain amino acid sequence that is at least 85%, 90%, 95%, 96%, 97%, 98%, 99% identical to the amino acid sequence:

(SEQ ID NO: 8697)
QVQLVQSGAEVKKPGSSVKVSCKASPRGFYGYVMHWVRQAPGQGLEWMGF
INPYTNDIQYNERFRGRVTITSDKSTTTAYMELSSLRSEDTAVYYCARGN
GKWGDGAYRFFDLWGQGTLVTVSSASTKGPSVFPLAPSSKSTSGGTAALG
CLVKDYFPEPVTVSWNSGALTSGVHTFPAVLQSSGLYSLSSVVTVPSSSL
KPKDTLMISRTPEVTCVVVDVSHEDPEVKFNWYVDGVEVHNAKTKPREEQ
GTQTYICNVNHKPSNTKVDKRVEPKSCDKTHTCPPCPAPELLGGPSVFLF
PPYASTYRVVSVLTVLHQDWLNGKEYKCKVSNKALPAPIEKTISKAKGQP
REPQVYTLPPCREEMTKNQVSLWCLVKGFYPSDIAVEWESNGQPENNYKT
TPPVLDSDGSFFLYSKLTVDKSRWQQGNVFSCSVMHEALHNHYTQKSLSL
SPGK.

In some embodiments, the bispecific antibody comprises: (i) a first antigen binding domain comprising an scFv that binds to TRBC2 domain, and may comprise a light chain amino acid sequence that is at least 85%, 90%, 95%, 96%, 97%, 98%, 99% identical to the amino acid sequence:

(SEQ ID NO: 8002)
DVVMTQSPLSLPVTLGQPASISCRSSENLVHSNGRTYLQWYQQRPGQSPR
LLIYRVSNRFPGVPDRFSGSGSGTDFTLKISRVEAEDVGVYFCSQSSLEP
YTFGGGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC.

In some embodiments, the bispecific antibody comprises: (ii) second antigen binding domain that binds to NKp30, comprising an scFv that binds to NKp30 having a sequence that is at least 85%, 90%, 95%, 96%, 97%, 98%, 99% identical to the amino acid sequence:

(SEQ ID NO: 8003)
EIQLLESGGGLVQPGGSLRLSCAVSGFSITTTGYHWNWVRQAPGKGLEWV
GYIYSSGSTSYNPSLKSRFTISRDTSKNTFYLQMNSLRAEDTAVYYCARG
DWHYFDYWGQGTMVTVSSGGGGSGGGGSGGGGSGGGGSDSVTTQSPLSLP
VTLGQPASISCSGEKLSDKYVHWYQQRPGQSPRMLIYENDRRPSGVPDRF
SGSNSGNDATLKISRVEAEDVGVYFCQFWDSTNSAVFGGGTKVEIKDKTH
TCPPCPAPELLGGPSVFLFPPKPKDTLMISRTPEVTCVVVDVSHEDPEVK
FNWYVDGVEVHNAKTKPREEQYASTYRVVSVLTVLHQDWLNGKEYKCKVS
NKALPAPIEKTISKAKGQPREPQVCTLPPSREEMTKNQVSLSCAVKGFYP
SDIAVEWESNGQPENNYKTTPPVLDSDGSFFLVSKLTVDKSRWQQGNVFS
CSVMHEALHNHYTQKSLSLSPGK.

In some embodiments, the first antigen binding domain has a higher affinity for a T cell receptor comprising TRBC2 than for a T cell receptor not comprising TRBC2, optionally wherein the KD for the binding between the first antigen binding domain and TRBC2 is no more than 40%, 30%, 20%, 10%, 1%, 0.1%, or 0.01% of the KD for the binding between the first antigen binding domain and a T cell receptor not comprising TRBC2. In some embodiments, the first antigen binding domain has a higher affinity for a T cell receptor comprising TRBC2 than for a T cell receptor comprising TCRBC1, optionally wherein the K_{D} for the binding between the first antigen binding domain and TRBC2 is no more than 40%, 30%, 20%, 10%, 1%, 0.1%, or 0.01% of the K_{D} for the binding between the first antigen binding domain and a T cell receptor comprising TCRBC1. In some embodiments, binding of the first antigen binding domain to TRBC2 on a lymphoma cell or lymphocyte, e.g., T cell, does not appreciably activate the lymphoma cell or lymphocyte, e.g., T cell, e.g., as measured by T cell proliferation, expression of a T cell activation marker (e.g., CD69 or CD25), and/or expression of a cytokine (e.g., TNFα and IFNγ). In some embodiments, the multifunctional molecule does not activate NK cells or does not substantially activate NK cells in the absence of a TRBC2-expressing cell.

In some embodiments, the multifunctional molecule binds to TRBC2 monovalently.

Antibody Molecules that Bind to TRBC1/TRBC2 and NKp30

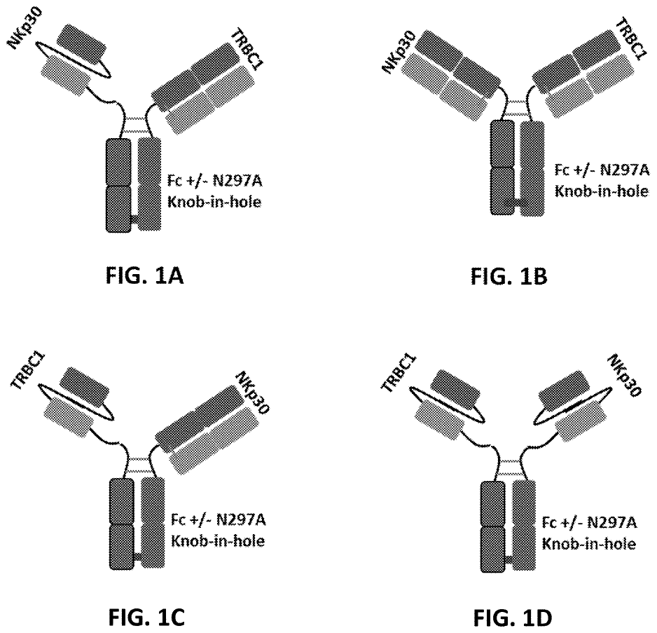

In some embodiments, the disclosure features a multifunctional antibody molecule that binds to TRBC1 and NKp30. In some embodiments, the multifunctional antibody molecule comprises a configuration shown in any of FIGS. 1A-1D. In some embodiments, the multifunctional antibody molecule comprises an anti-TRBC1 Fab. In some embodiments, the multifunctional antibody molecule comprises an anti-TRBC1 scFv. In some embodiments, the multifunctional antibody molecule comprises an anti-NKp30 Fab. In some embodiments, the multifunctional antibody molecule comprises an anti-NKp30 scFv. In some embodiments, the multifunctional antibody molecule comprises an anti-TRBC1 Fab and an anti-NKp30 scFv, e.g., comprises a configuration shown in FIG. 1A. In some embodiments, the multifunctional antibody molecule comprises an anti-TRBC1 Fab and an anti-NKp30 Fab, e.g., comprises a configuration shown in FIG. 1B. In some embodiments, the multifunctional antibody molecule comprises an anti-NKp30 Fab and an anti-TRBC1 scFv, e.g., comprises a configuration shown in FIG. 1C. In some embodiments, the multifunctional antibody molecule comprises an anti-TRBC1 scFv and an anti-NKp30 scFv, e.g., comprises a configuration shown in FIG. 1D. In some embodiments, the multifunctional antibody molecule comprises an anti-TRBC1 antigen binding domain disclosed herein, e.g., an anti-TRBC1 antigen binding domain disclosed in Table 1, Table 2A or Table 2B, Table 3A or Table 3B, Table 4, Table 7, Table 8. In some embodiments, the multifunctional antibody molecule comprises an anti-NKp30 antigen binding domain disclosed herein, e.g., an anti-NKp30 antigen binding domain disclosed in Table 16, Table 17, Table 20A or Table 20B, Table 21A or Table 21B, Table 22, Table 23A or Table 23B, Table 24, Table 25, Table 26.

In some embodiments, exemplary multifunctional antibody molecules that bind to TRBC1 and NKp30 are disclosed in Table 16.

In some embodiments, the multifunctional antibody molecule comprises an anti-TRBC1 VH of SEQ ID NO: 7351 (or a sequence having at least 85%, 90%, 95%, or 99% identity thereto), an anti-TRBC1 VL of SEQ ID NO: 258 (or a sequence having at least 85%, 90%, 95%, or 99% identity thereto), an anti-NKp30 VH of SEQ ID NO: 8030 (or a sequence having at least 85%, 90%, 95%, or 99% identity thereto), and an anti-NKp30 VL of SEQ ID NO: 13 (or a sequence having at least 85%, 90%, 95%, or 99% identity thereto). In some embodiments, the anti-TRBC1/NKp30 antibody molecule comprises an anti-TRBC1 VH of SEQ ID NO: 7351 (or a sequence having at least 85%, 90%, 95%, or 99% identity thereto), an anti-TRBC1 VL of SEQ ID NO: 258 (or a sequence having at least 85%, 90%, 95%, or 99% identity thereto), and an anti-NKp30 scFv of SEQ ID NO: 7311 (or a sequence having at least 85%, 90%, 95%, or 99% identity thereto). In some embodiments, the anti-TRBC1/NKp30 antibody molecule comprises SEQ ID NOs: 7382, 4677, 7380, 4678, and 7383 (or a sequence having at least 85%, 90%, 95%, or 99% identity thereto).

In some embodiments, the multifunctional antibody molecule comprises an anti-TRBC1 VH of SEQ ID NO: 253 (or a sequence having at least 85%, 90%, 95%, or 99% identity thereto), an anti-TRBC1 VL of SEQ ID NO: 258 (or a sequence having at least 85%, 90%, 95%, or 99% identity thereto), an anti-NKp30 VH of SEQ ID NO: 8030 (or a sequence having at least 85%, 90%, 95%, or 99% identity thereto), and an anti-NKp30 VL of SEQ ID NO: 13 (or a sequence having at least 85%, 90%, 95%, or 99% identity thereto). In some embodiments, the anti-TRBC1/NKp30 antibody molecule comprises an anti-TRBC1 VH of SEQ ID NO: 253 (or a sequence having at least 85%, 90%, 95%, or 99% identity thereto), an anti-TRBC1 VL of SEQ ID NO: 258 (or a sequence having at least 85%, 90%, 95%, or 99% identity thereto), and an anti-NKp30 scFv of SEQ ID NO: 7311 (or a sequence having at least 85%, 90%, 95%, or 99% identity thereto). In some embodiments, the anti-TRBC1/NKp30 antibody molecule comprises SEQ ID NOs: 7379, 4675, 7380, 4678, and 7383 (or a sequence having at least 85%, 90%, 95%, or 99% identity thereto).

In some embodiments, the multifunctional antibody molecule comprises an anti-TRBC1 VH of SEQ ID NO: 7351 (or a sequence having at least 85%, 90%, 95%, or 99% identity thereto), an anti-TRBC1 VL of SEQ ID NO: 258 (or a sequence having at least 85%, 90%, 95%, or 99% identity thereto), an anti-NKp30 VH of SEQ ID NO: 8030 (or a sequence having at least 85%, 90%, 95%, or 99% identity thereto), and an anti-NKp30 VL of SEQ ID NO: 7305 (or a sequence having at least 85%, 90%, 95%, or 99% identity thereto). In some embodiments, the anti-TRBC1/NKp30 antibody molecule comprises an anti-TRBC1 VH of SEQ ID NO: 7351 (or a sequence having at least 85%, 90%, 95%, or 99% identity thereto), an anti-TRBC1 VL of SEQ ID NO: 258 (or a sequence having at least 85%, 90%, 95%, or 99% identity thereto), and an anti-NKp30 scFv of SEQ ID NO: 7310 (or a sequence having at least 85%, 90%, 95%, or 99% identity thereto). In some embodiments, the anti-TRBC1/NKp30 antibody molecule comprises SEQ ID NOs: 7382, 4677, 7380, 4679, and 7384 (or a sequence having at least 85%, 90%, 95%, or 99% identity thereto).

In some embodiments, the multifunctional antibody molecule comprises an anti-TRBC1 VH of SEQ ID NO: 253 (or a sequence having at least 85%, 90%, 95%, or 99% identity thereto), an anti-TRBC1 VL of SEQ ID NO: 258 (or a sequence having at least 85%, 90%, 95%, or 99% identity thereto), an anti-NKp30 VH of SEQ ID NO: 8030 (or a sequence having at least 85%, 90%, 95%, or 99% identity thereto), and an anti-NKp30 VL of SEQ ID NO: 7305 (or a sequence having at least 85%, 90%, 95%, or 99% identity thereto). In some embodiments, the anti-TRBC1/NKp30 antibody molecule comprises an anti-TRBC1 VH of SEQ ID NO: 253 (or a sequence having at least 85%, 90%, 95%, or 99% identity thereto), an anti-TRBC1 VL of SEQ ID NO: 258 (or a sequence having at least 85%, 90%, 95%, or 99% identity thereto), and an anti-NKp30 scFv of SEQ ID NO: 7310 (or a sequence having at least 85%, 90%, 95%, or 99% identity thereto). In some embodiments, the anti-TRBC1/NKp30 antibody molecule comprises SEQ ID NOs: 7379, 4675, 7380, 4679, and 7384 (or a sequence having at least 85%, 90%, 95%, or 99% identity thereto).

In some embodiments provided herein is an antibody or a fragment thereof that binds to a TRBC1 molecule, wherein the antibody or fragment thereof that binds to the TRBC1 comprises a heavy chain comprising an HC-CDR1, having a sequence GYVMH (SEQ ID NO 8643); an HC-CDR2, having a sequence of FINPYNDDIQSNERFRG (SEQ ID NO: 201); and an HC-CDR3, having a sequence of GAGYNFDGAYRFFDF (SEQ ID NO: 202); and a light chain comprising an LC-CDR1 of RSSQRLVHSNGNTYLH (SEQ ID NO: 8646), an LC-CDR2 of RVSNRFP (SEQ ID NO: 224), an LC-CDR3 of SQSTHVPYT (SEQ ID NO: 225).

In some embodiments provided herein is an antibody or a fragment thereof that binds to a TRBC1 molecule, wherein the antibody or fragment thereof that binds to the TRBC1 comprises a heavy chain comprising an HC-CDR1, having a sequence GYVMH (SEQ ID NO 8643); an HC-CDR2, having a sequence FIIPIFGTANYAQKFQG (SEQ ID NO: 7355) and an HC-CDR3, having a sequence GAGYNFDGAYRFFDF (SEQ ID NO: 202); and a light chain comprising an LC-CDR1 having a sequence, RSSQRLVHSNGNTYLH (SEQ ID NO: 8651), an LC-CDR2 having sequence RVSNRFP (SEQ ID NO: 224), and an LC-CDR3 having a sequence SQSTHVPYT (SEQ ID NO: 225).

In some embodiments, provided herein is an antibody or a fragment thereof that binds to a TRBC1 molecule, wherein the antibody or fragment thereof that binds to the TRBC1 comprises a heavy chain comprising an HC-CDR1, having a sequence GYVMH (SEQ ID NO 8643); an HC-CDR2, having a sequence FINPYNDDIQSNERFRG (SEQ ID NO: 8654) and an HC-CDR3, having a sequence GAGYNFDGAYRFFDF (SEQ ID NO: 202); and a light chain comprising an LC-CDR1 having a sequence, RSSQRLVHSNGNTYLH (SEQ ID NO: 8656), an LC-CDR2 having sequence RVSNRFP (SEQ ID NO: 224), and an LC-CDR3 having a sequence SQSTHVPYT (SEQ ID NO: 225).

In some embodiments, provided herein is an antibody or a fragment thereof that binds to a TRBC1 molecule, wherein the antibody or fragment thereof that binds to the TRBC1 comprises a heavy chain comprising an HC-CDR1, having a sequence GYVMH (SEQ ID NO 8643); an HC-CDR2, having a sequence FIIPIFGTANYAQKFQG (SEQ ID NO: 7355) and an HC-CDR3, having a sequence GAGYNFDGAYRFFDF (SEQ ID NO: 202); and a light chain comprising an LC-CDR1 having a sequence, RSSQRLVHSNGNTYLH (SEQ ID NO: 8661), an LC-CDR2 having sequence RVSNRFP (SEQ ID NO: 224), and an LC-CDR3 having a sequence SQSTHVPYT (SEQ ID NO: 225).

In some embodiments, provided herein is an antibody or a fragment thereof that binds to a TRBC1 molecule, wherein the antibody or fragment thereof that binds to the TRBC1 comprises a heavy chain comprising an HC-CDR1, having a sequence GYVMH (SEQ ID NO 8643); an HC-CDR2, having a sequence FINPYNDDIQSNERFRG (SEQ ID NO: 8664) and an HC-CDR3, having a sequence GAGYNFDGAYRFFDF (SEQ ID NO: 202); and a light chain comprising an LC-CDR1 having a sequence, RSSQRLVHSNGNTYLH (SEQ ID NO: 8666), an LC-CDR2 having sequence RVSNRFP (SEQ ID NO: 224), and an LC-CDR3 having a sequence SQSTHVPYT (SEQ ID NO: 225).

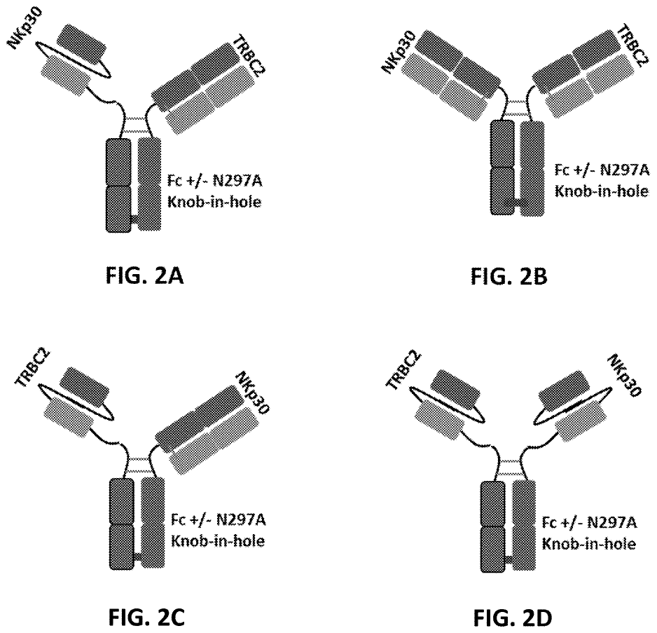

In some embodiments, the disclosure features a multifunctional antibody molecule that binds to both TRBC2 and NKp30. In some embodiments, the multifunctional antibody molecule comprises a configuration shown in any of FIGS. 2A-2D. In some embodiments, the multifunctional antibody molecule comprises an anti-TRBC2 Fab. In some embodiments, the multifunctional antibody molecule comprises an anti-TRBC2 scFv. In some embodiments, the multifunctional antibody molecule comprises an anti-NKp30 Fab. In some embodiments, the multifunctional antibody molecule comprises an anti-NKp30 scFv. In some embodiments, the multifunctional antibody molecule comprises an anti-TRBC2 Fab and an anti-NKp30 scFv, e.g., comprises a configuration shown in FIG. 2A. In some embodiments, the multifunctional antibody molecule comprises an anti-TRBC2 Fab and an anti-NKp30 Fab, e.g., comprises a configuration shown in FIG. 2B. In some embodiments, the multifunctional antibody molecule comprises an anti-NKp30 Fab and an anti-TRBC2 scFv, e.g., comprises a configuration shown in FIG. 2C. In some embodiments, the multifunctional antibody molecule comprises an anti-TRBC2 scFv and an anti-NKp30 scFv, e.g., comprises a configuration shown in FIG. 2D. In some embodiments, the multifunctional antibody molecule comprises an anti-TRBC2 antigen binding domain disclosed herein, e.g., an anti-TRBC2 antigen binding domain disclosed in Table 9A or Table 9B, Table 10, Table 11, Table 12, Table 13, Table 14, table 15, Table 17, Table 39. In some embodiments, the multifunctional antibody molecule comprises an anti-NKp30 antigen binding domain disclosed herein, e.g., an anti-NKp30 antigen binding domain disclosed in Table 20A or Table 20B, Table 22, Table 23A or Table 23B, Table 24, Table 25, Table 26, Table 21A or Table 21B, and Table 17.

In some embodiments, exemplary multifunctional antibody molecules that bind to TRBC2 and NKp30 are disclosed in Table 17.

In some embodiments, the multifunctional antibody molecule comprises an anti-TRBC2 VH of SEQ ID NO: 7420 (or a sequence having at least 85%, 90%, 95%, or 99% identity thereto), an anti-TRBC2 VL of SEQ ID NO: 7419 (or a sequence having at least 85%, 90%, 95%, or 99% identity thereto), an anti-NKp30 VH of SEQ ID NO: 8030 (or a sequence having at least 85%, 90%, 95%, or 99% identity thereto), and an anti-NKp30 VL of SEQ ID NO: 13 (or a sequence having at least 85%, 90%, 95%, or 99% identity thereto). In some embodiments, the anti-TRBC2/NKp30 antibody molecule comprises an anti-TRBC2 VH of SEQ ID NO: 7420 (or a sequence having at least 85%, 90%, 95%, or 99% identity thereto), an anti-TRBC2 VL of SEQ ID NO: 7419 (or a sequence having at least 85%, 90%, 95%, or 99% identity thereto), and an anti-NKp30 scFv of SEQ ID NO: 7311 (or a sequence having at least 85%, 90%, 95%, or 99% identity thereto). In some embodiments, the anti-TRBC2/NKp30 antibody molecule comprises SEQ ID NOs: 7438, 7439, 4678, and 7383 (or a sequence having at least 85%, 90%, 95%, or 99% identity thereto).

In some embodiments, the multifunctional antibody molecule comprises an anti-TRBC2 VH of SEQ ID NO: 7423 (or a sequence having at least 85%, 90%, 95%, or 99% identity thereto), an anti-TRBC2 VL of SEQ ID NO: 7419 (or a sequence having at least 85%, 90%, 95%, or 99% identity thereto), an anti-NKp30 VH of SEQ ID NO: 8030 (or a sequence having at least 85%, 90%, 95%, or 99% identity thereto), and an anti-NKp30 VL of SEQ ID NO: 13 (or a sequence having at least 85%, 90%, 95%, or 99% identity thereto). In some embodiments, the anti-TRBC2/NKp30 antibody molecule comprises an anti-TRBC2 VH of SEQ ID NO: 7423 (or a sequence having at least 85%, 90%, 95%, or 99% identity thereto), an anti-TRBC2 VL of SEQ ID NO: 7419 (or a sequence having at least 85%, 90%, 95%, or 99% identity thereto), and an anti-NKp30 scFv of SEQ ID NO: 7311 (or a sequence having at least 85%, 90%, 95%, or 99% identity thereto). In some embodiments, the anti-TRBC2/NKp30 antibody molecule comprises SEQ ID NOs: 4681, 7440, 7439, 4678, and 7383 (or a sequence having at least 85%, 90%, 95%, or 99% identity thereto).

In some embodiments, the multifunctional antibody molecule comprises an anti-TRBC2 VH of SEQ ID NO: 7420 (or a sequence having at least 85%, 90%, 95%, or 99% identity thereto), an anti-TRBC2 VL of SEQ ID NO: 7419 (or a sequence having at least 85%, 90%, 95%, or 99% identity thereto), an anti-NKp30 VH of SEQ ID NO: 8030 (or a sequence having at least 85%, 90%, 95%, or 99% identity thereto), and an anti-NKp30 VL of SEQ ID NO: 7305 (or a sequence having at least 85%, 90%, 95%, or 99% identity thereto). In some embodiments, the anti-TRBC2/NKp30 antibody molecule comprises an anti-TRBC2 VH of SEQ ID NO: 7420 (or a sequence having at least 85%, 90%, 95%, or 99% identity thereto), an anti-TRBC2 VL of SEQ ID NO: 7419 (or a sequence having at least 85%, 90%, 95%, or 99% identity thereto), and an anti-NKp30 scFv of SEQ ID NO: 7310 (or a sequence having at least 85%, 90%, 95%, or 99% identity thereto). In some embodiments, the anti-TRBC2/NKp30 antibody molecule comprises SEQ ID NOs: 7438, 7439, 4679, and 7384 (or a sequence having at least 85%, 90%, 95%, or 99% identity thereto).

In some embodiments, the multifunctional antibody molecule comprises an anti-TRBC2 VH of SEQ ID NO: 7423 (or a sequence having at least 85%, 90%, 95%, or 99% identity thereto), an anti-TRBC2 VL of SEQ ID NO: 7419 (or a sequence having at least 85%, 90%, 95%, or 99% identity thereto), an anti-NKp30 VH of SEQ ID NO: 8030 (or a sequence having at least 85%, 90%, 95%, or 99% identity thereto), and an anti-NKp30 VL of SEQ ID NO: 7305 (or a sequence having at least 85%, 90%, 95%, or 99% identity thereto). In some embodiments, the anti-TRBC2/NKp30 antibody molecule comprises an anti-TRBC2 VH of SEQ ID NO: 7423 (or a sequence having at least 85%, 90%, 95%, or 99% identity thereto), an anti-TRBC2 VL of SEQ ID NO: 7419 (or a sequence having at least 85%, 90%, 95%, or 99% identity thereto), and an anti-NKp30 scFv of SEQ ID NO: 7310 (or a sequence having at least 85%, 90%, 95%, or 99% identity thereto). In some embodiments, the anti-TRBC2/NKp30 antibody molecule comprises SEQ ID NOs: 4681, 7440, 7439, 4979, and 7384 (or a sequence having at least 85%, 90%, 95%, or 99% identity thereto).

### Multifunctional Antibody Effector Function and Fc Variants

In some embodiments, the multifunctional molecule (e.g., an anti-TRBC1/NKp30 antibody molecule, an anti-TRBC2/NKp30 antibody molecule, or an anti-TRBV/NKp30 antibody molecule) disclosed herein comprises an Fc region, e.g., as described herein. In some embodiments, the Fc region is a wildtype Fc region, e.g., a wildtype human Fc region. In some embodiments, the Fc region comprises a variant, e.g., an Fc region comprising an addition, substitution, or deletion of at least one amino acid residue in the Fc region which results in, e.g., reduced or ablated affinity for at least one Fc receptor.

The Fc region of an antibody interacts with a number of receptors or ligands including Fe Receptors (e.g., FcγRI, FcγRIIA, FcγRIIIA), the complement protein CIq, and other molecules such as proteins A and G. These interactions are essential for a variety of effector functions and downstream signaling events including: antibody dependent cell-mediated cytotoxicity (ADCC), Antibody-dependent cellular phagocytosis (ADCP) and complement dependent cytotoxicity (CDC).

In some embodiments, the multifunctional molecule (e.g., an anti-TRBC1/NKp30 antibody molecule, an anti-TRBC2/NKp30 antibody molecule, or an anti-TRBV/NKp30 antibody molecule) comprising a variant Fc region has reduced, e.g., ablated, affinity for an Fc receptor, e.g., an Fc receptor described herein. In some embodiments, the reduced affinity is compared to an otherwise similar antibody with a wildtype Fc region.

In some embodiments, the multifunctional molecule (e.g., an anti-TRBC1/NKp30 antibody molecule, an anti-TRBC2/NKp30 antibody molecule, or an anti-TRBV/NKp30 antibody molecule) comprising a variant Fc region has one or more of the following properties: (1) reduced effector function (e.g., reduced ADCC, ADCP and/or CDC); (2) reduced binding to one or more Fc receptors; and/or (3) reduced binding to C1q complement. In some embodiments, the reduction in any one, or all of properties (1)-(3) is compared to an otherwise similar antibody with a wildtype Fc region.

In some embodiments, the multifunctional molecule (e.g., an anti-TRBC1/NKp30 antibody molecule, an anti-TRBC2/NKp30 antibody molecule, or an anti-TRBV/NKp30 antibody molecule) comprising a variant Fc region has reduced affinity to a human Fc receptor, e.g., FcγR I, FcγR II and/or FcγR III. In some embodiments, the multifunctional molecule (e.g., an anti-TRBC1/NKp30 antibody molecule or an anti-TRBC2/NKp30 antibody molecule) comprising a variant Fc region comprises a human IgG1 region or a human IgG4 region.

Exemplary Fc region variants are provided in Table 18 and also disclosed in Saunders O, (2019) Frontiers in Immunology; vol 10, article1296, the entire contents of which is hereby incorporated by reference.

In some embodiments, the multifunctional molecule (e.g., an anti-TRBC1/NKp30 antibody molecule, an anti-TRBC2/NKp30 antibody molecule, or an anti-TRBV/NKp30 antibody molecule) disclosed herein has a light chain constant region chosen from, e.g., the light chain constant regions of kappa or lambda, preferably kappa (e.g., human kappa). In some embodiments, the constant region is altered, e.g., mutated, to modify the properties of the multifunctional molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217 or 218).

In some embodiments, the agent and/or multispecific molecules described herein comprises an Fc region comprising a first Fe subunit and a second Fe subunit. In some embodiments, the Fc region is a wild-type Fc region. In some embodiments, the Fc region comprises N297.

In some embodiments, the Fc region comprises one or more amino acid substitutions relative to a wild-type Fc region selected from the group consisting of heavy chain constant regions of human IgE, IgM, IgG1, IgG2, IgG3, IgG4, IgA1, and IgA2. In some embodiments, the Fc region comprises an Fc-silencing mutation, which Fc-silencing mutation decreases an antibody-directed cytotoxicity effector function. In some embodiments, the Fc region comprises a mutation to enhance an effector function.

In some embodiments, an Fc region of an agent, multispecific molecule, or any combination thereof described herein may comprise one or more amino acid substitutions relative to a wild-type Fc region. The wild-type Fc region can comprise an Fc region of a heavy chain constant region. In some embodiments, the heavy chain constant region can be a wild-type IgE, IgM, IgG1, IgG2, IgG3, IgG4, IgA1, or IgA2. In some embodiments, the Fc region comprises at least one amino acid substitution relative to a wild-type heavy chain constant region as set forth in any one of SEQ ID NOs: 42 and 4663-4670. In some embodiments, the Fc region comprises a Fc region with at least about 90%, at least about 91%, at least about 92%, at least about 93%, at least about 94%, at least about 95%, at least about 96%, at least about 97%, at least about 98%, at least about 98.5%, at least about 99%, at least about 99.5%, at least about 99.9%, or greater than about 99.9% sequence identity to an amino acid sequence as set forth in any one of SEQ ID NOs: 42 and 4663-4670. In some embodiments, the Fc region comprises an amino acid sequence as set forth in any one of SEQ ID NOs: 42 and 4663-4670. In some embodiments, the Fc region comprises a Fc region with at least about 90%, at least about 91%, at least about 92%, at least about 93%, at least about 94%, at least about 95%, at least about 96%, at least about 97%, at least about 98%, at least about 98.5%, at least about 99%, at least about 99.5%, at least about 99.9%, or greater than about 99.9% sequence identity to an amino acid sequence as set forth in Table 41. In some embodiments, the Fc region comprises an amino acid sequence as set forth in Table 41.

In some embodiments, a Fc region of an agent, multispecific molecule, or any combination thereof described herein may comprise one or more mutations selected from the group consisting of E233P, L234V, L235A, P329A, S267K, G237A, P238S, H268A, A330S, P331S, K322A, D265A, D265S, L235E, G236R, S239K, A327G, V273E, T299A, Y300S, where amino acid positioning is relative to an one of SEQ ID NO: 42, 3645, or 4663-4670.

TABLE 41
Immunoglobulin Fc Region Sequences.

| | | SEQ | |
|---|---|---|---|
| | UniProt | ID | |
| Description | ID | NO. | Sequence |
| IGG1_HUMAN | P0DOX5 | 4663 | QVQLVQSGGGVVQPGRSLRLSCAASGFTFSRYTIHWVRQAPGKGL |
| Immuno- | | | EWVAVMSYNGNNKHYADSVNGRFTISRNDSKNTLYLNMNSLRPE |
| globulin | | | DTAVYYCARIRDTAMFFAHWGQGTLVTVSSASTKGPSVFPLAPSS |
| gamma-1 | | | KSTSGGTAALGCLVKDYFPEPVTVSWNSGALTSGVHTFPAVLQSS |
| heavy chain | | | GLYSLSSVVTVPSSSLGTQTYICNVNHKPSNTKVDKKVEPKSCDKT |
| | | | HTCPPCPAPELLGGPSVFLFPPKPKDTLMISRTPEVTCVVVDVSHE |
| | | | DPEVKFNWYVDGVEVHNAKTKPREEQYNSTYRVVSVLTVLHQD |
| | | | WLNGKEYKCKVSNKALPAPIEKTISKAKGQPREPQVYTLPPSRDEL |
| | | | TKNQVSLTCLVKGFYPSDIAVEWESNGQPENNYKTTPPVLDSDGS |
| | | | FFLYSKLTVDKSRWQQGNVFSCSVMHEALHNHYTQKSLSLSPGK |
| IGHG2_HUMAN | P01859 | 4664 | ASTKGPSVFPLAPCSRSTSESTAALGCLVKDYFPEPVTVSWNSGAL |
| Immuno- | | | TSGVHTFPAVLQSSGLYSLSSVVTVPSSNFGTQTYTCNVDHKPSNT |
| globulin | | | KVDKTVERKCCVECPPCPAPPVAGPSVFLFPPKPKDTLMISRTPEV |
| heavy | | | TCVVVDVSHEDPEVQFNWYVDGVEVHNAKTKPREEQFNSTFRVV |
| constant | | | SVLTVVHQDWLNGKEYKCKVSNKGLPAPIEKTISKTKGQPREPQV |
| gamma | | | YTLPPSREEMTKNQVSLTCLVKGFYPSDISVEWESNGQPENNYKT |
| | | | TPPMLDSDGSFFLYSKLTVDKSRWQQGNVFSCSVMHEALHNHYT |
| | | | QKSLSLSPELQLEESCAEAQDGELDGLWTTITIFITLFLLSVCYSATI |
| | | | TFFKVKWIFSSVVDLKQTIVPDYRNMIRQGA |
| IGHG3_HUMAN | P01860 | 4665 | ASTKGPSVFPLAPCSRSTSGGTAALGCLVKDYFPEPVTVSWNSGA |
| Immuno- | | | LTSGVHTFPAVLQSSGLYSLSSVVTVPSSSLGTQTYTCNVNHKPSN |
| globulin | | | TKVDKRVELKTPLGDTTHTCPRCPEPKSCDTPPPCPRCPEPKSCDT |
| heavy | | | PPPCPRCPEPKSCDTPPPCPRCPAPELLGGPSVFLFPPKPKDTLMISR |
| constant | | | TPEVTCVVVDVSHEDPEVQFKWYVDGVEVHNAKTKPREEQYNST |
| gamma | | | FRVVSVLTVLHQDWLNGKEYKCKVSNKALPAPIEKTISKTKGQPR |
| | | | EPQVYTLPPSREEMTKNQVSLTCLVKGFYPSDIAVEWESSGQPEN |
| | | | NYNTTPPMLDSDGSFFLYSKLTVDKSRWQQGNIFSCSVMHEALHN |
| | | | RFTQKSLSLSPELQLEESCAEAQDGELDGLWTTITIFITLFLLSVCYS |
| | | | ATVTFFKVKWIFSSVVDLKQTIIPDYRNMIGQGA |
| IGHG4_HUMAN | P01861 | 4666 | ASTKGPSVFPLAPCSRSTSESTAALGCLVKDYFPEPVTVSWNSGAL |
| Immuno- | | | TSGVHTFPAVLQSSGLYSLSSVVTVPSSSLGTKTYTCNVDHKPSNT |
| globulin | | | KVDKRVESKYGPPCPSCPAPEFLGGPSVFLFPPKPKDTLMISRTPEV |
| heavy | | | TCVVVDVSQEDPEVQFNWYVDGVEVHNAKTKPREEQFNSTYRVV |
| constant | | | SVLTVLHQDWLNGKEYKCKVSNKGLPSSIEKTISKAKGQPREPQV |
| gamma | | | YTLPPSQEEMTKNQVSLTCLVKGFYPSDIAVEWESNGQPENNYKT |
| | | | TPPVLDSDGSFFLYSRLTVDKSRWQEGNVFSCSVMHEALHNHYTQ |
| | | | KSLSLSLELQLEESCAEAQDGELDGLWTTITIFITLFLLSVCYSATVT |
| | | | FFKVKWIFSSVVDLKQTIVPDYRNMIRQGA |
| IGHA1_HUMAN | P01876 | 4667 | ASPTSPKVFPLSLCSTQPDGNVVIACLVQGFFPQEPLSVTWSESGQ |
| Immuno- | | | GVTARNFPPSQDASGDLYTTSSQLTLPATQCLAGKSVTCHVKHYT |
| globulin | | | NPSQDVTVPCPVPSTPPTPSPSTPPTPSPSCCHPRLSLHRPALEDLLL |
| heavy | | | GSEANLTCTLTGLRDASGVTFTWTPSSGKSAVQGPPERDLCGCYS |
| constant | | | VSSVLPGCAEPWNHGKTFTCTAAYPESKTPLTATLSKSGNTFRPEV |
| alpha | | | HLLPPPSEELALNELVTLTCLARGFSPKDVLVRWLQGSQELPREKY |
| | | | LTWASRQEPSQGTTTFAVTSILRVAAEDWKKGDTFSCMVGHEALP |
| | | | LAFTQKTIDRLADWQMPPPYVVLDLPQETLEEETPGANLWPTTITF |
| | | | LTLFLLSLFYSTALTVTSVRGPSGNREGPQY |
| IGHA2_HUMAN | P01877 | 4668 | ASPTSPKVFPLSLDSTPQDGNVVVACLVQGFFPQEPLSVTWSESGQ |
| Immuno- | | | NVTARNFPPSQDASGDLYTTSSQLTLPATQCPDGKSVTCHVKHYT |
| globulin | | | NSSQDVTVPCRVPPPPPCCHPRLSLHRPALEDLLLGSEANLTCTLT |
| heavy | | | GLRDASGATFTWTPSSGKSAVQGPPERDLCGCYSVSSVLPGCAQP |
| constant | | | WNHGETFTCTAAHPELKTPLTANITKSGNTFRPEVHLLPPPSEELA |
| alpha | | | LNELVTLTCLARGFSPKDVLVRWLQGSQELPREKYLTWASRQEPS |
| | | | QGTTTYAVTSILRVAAEDWKKGETFSCMVGHEALPLAFTQKTIDR |
| | | | MAGSCCVADWQMPPPYVVLDLPQETLEEETPGANLWPTTITFLTL |
| | | | FLLSLFYSTALTVTSVRGPSGKREGPQY |
| IGHM_HUMAN | P01871 | 4669 | GSASAPTLFPLVSCENSPSDTSSVAVGCLAQDFLPDSITFSWKYKN |
| Immuno- | | | NSDISSTRGFPSVLRGGKYAATSQVLLPSKDVMQGTDEHVVCKVQ |
| globulin | | | HPNGNKEKNVPLPVIAELPPKVSVFVPPRDGFFGNPRKSKLICQAT |
| heavy | | | GFSPRQIQVSWLREGKQVGSGVTTDQVQAEAKESGPTTYKVTSTL |
| constant mu | | | TIKESDWLGQSMFTCRVDHRGLTFQQNASSMCVPDQDTAIRVFAI |
| | | | PPSFASIFLTKSTKLTCLVTDLTTYDSVTISWTRQNGEAVKTHTNIS |
| | | | ESHPNATFSAVGEASICEDDWNSGERFTCTVTHTDLPSPLKQTISRP |
| | | | KGVALHRPDVYLLPPAREQLNLRESATITCLVTGFSPADVFVQWM |
| | | | QRGQPLSPEKYVTSAPMPEPQAPGRYFAHSILTVSEEEWNTGETYT |
| | | | CVVAHEALPNRVTERTVDKSTEGEVSADEEGFENLWATASTFIVL |
| | | | FLLSLFYSTTVTLFKVKPADVFVQWMQRGEPLSPEKYVTSAPMPE |
| | | | PQAPGRYFAHSILTVSEEEWNTGQTYTCVVAHEALPNRVTERTVD |
| | | | KSTGKPTLYNVSLVMSDTAGTCY |
| IGHE_HUMAN | P01854 | 4670 | ASTQSPSVFPLTRCCKNIPSNATSVTLGCLATGYFPEPVMVTWDTG |
| Immuno- | | | SLNGTTMTLPATTLTLSGHYATISLLTVSGAWAKQMFTCRVAHTP |
| globulin | | | SSTDWVDNKTFSVCSRDFTPPTVKILQSSCDGGGHFPPTIQLLCLVS |
| heavy | | | GYTPGTINITWLEDGQVMDVDLSTASTTQEGELASTQSELTLSQKH |
| constant | | | WLSDRTYTCQVTYQGHTFEDSTKKCADSNPRGVSAYLSRPSPFDL |
| epsilon | | | FIRKSPTITCLVVDLAPSKGTVNLTWSRASGKPVNHSTRKEEKQRN |
| | | | GTLTVTSTLPVGTRDWIEGETYQCRVTHPHLPRALMRSTTKTSGPR |
| | | | AAPEVYAFATPEWPGSRDKRTLACLIQNFMPEDISVQWLHNEVQL |
| | | | PDARHSTTQPRKTKGSGFFVFSRLEVTRAEWEQKDEFICRAVHEA |
| | | | ASPSQTVQRAVSVNPGLAGGSAQSQRAPDRVLCHSGQQQGLPRA |
| | | | AGGSVPHPRCHCGAGRADWPGPPELDVCVEEAEGEAPWTWTGL |
| | | | CIFAALFLLSVSYSAAITLLMVQRFLSATRQGRPQTSLDYTNVLQP |
| | | | HA |

In some embodiments, an Fc region can comprise a LALA mutation (L234A/L235A), a LALAPG mutation (L234A/L235A/P329G), an aglycosyl mutation (N297A, N297G, N297H, N297Q, or N297S), a STR mutation (2345/235T/236 R), or any combination thereof, where numbering is according to EU numbering system and amino acid positions correspond to SEQ ID NO: 3645.

In some embodiments, a Fc region of an agent and/or multispecific molecule described herein may comprise (i) L234S/L235T/G236R, corresponding to positions of a IgG1 sequence provided herein, (ii) V234S/A235T/*236 R, corresponding to positions of a IgG2 sequence provided herein, (iii) F234S/L235T/G236R, corresponding to positions of a IgG4 sequence provided herein, or any combination thereof. In some embodiments, Fc regions that can provide low ADCP, ADCC, and/or CDC activity can be E233P/L234A/L235A/G236*/P329A, E233P/L234V/G236*/S267K, L234A/L235A/P329S, L234A/L235A/D265S, L235G/G236R, or any combination thereof. The symbol “*” can designate a deleted residue.

In some embodiments, the multifunctional or multispecific molecule described herein comprises a Fc region that does not comprise a mutation.

In some embodiments, a multispecific molecule described herein comprising a NK cell engager may comprise a Fe mutation. In some embodiments, the Fe mutation can be a Fc-silencing mutation. In some embodiments, the Fe mutation can be mutation that enhances an effector function. In some embodiments, a multispecific molecule described herein comprising a NK cell engager may not comprise a Fe mutation. In some embodiments, a multispecific molecule described herein comprising a T cell engager may comprise a Fe mutation. In some embodiments, the Fe mutation can be a Fc-silencing mutation. In some embodiments, the Fe mutation can be mutation that enhances an effector function. In some embodiments, a multispecific molecule described herein comprising a T cell engager may not comprise a Fe mutation. In some embodiments, a multispecific molecule described herein comprising a B cell engager may comprise a Fe mutation. In some embodiments, the Fe mutation can be a Fc-silencing mutation. In some embodiments, the Fe mutation can be mutation that enhances an effector function. In some embodiments, a multispecific molecule described herein comprising a B cell engager may not comprise a Fe mutation. In some embodiments, a multispecific molecule described herein comprising a B cell engager may comprise a Fe mutation. In some embodiments, the Fe mutation can be a Fc-silencing mutation. In some embodiments, the Fe mutation can be mutation that enhances an effector function. In some embodiments, a multispecific molecule described herein comprising a dendritic cell engager may not comprise a Fe mutation. In some embodiments, a multispecific molecule described herein comprising a B cell engager may comprise a Fe mutation. In some embodiments, the Fe mutation can be a Fc-silencing mutation. In some embodiments, the Fe mutation can be mutation that enhances an effector function. In some embodiments, a multispecific molecule described herein comprising a macrophage cell engager may not comprise a Fe mutation.

In some embodiments, the multifunctional molecule (e.g., an anti-TRBC1/NKp30 antibody molecule, an anti-TRBC2/NKp30 antibody molecule, or an anti-TRBV/NKp30 antibody molecule) comprises any one or all, or any combination of Fc region variants, e.g., mutations, disclosed in Table 18. In some embodiments, the multifunctional molecule (e e.g., an anti-TRBC1/NKp30 antibody molecule, an anti-TRBC2/NKp30 antibody molecule, or an anti-TRBV/NKp30 antibody molecule) comprises an Asn297Ala (N297A) mutation. In some embodiments, the multifunctional molecule (e.g., an anti-TRBC1/NKp30 antibody molecule, an anti-TRBC2/NKp30 antibody molecule, or an anti-TRBV/NKp30 antibody molecule) comprises a Leu234Ala/Leu235Ala (LALA) mutation.

### Antibody Molecules Targeting TRBC1

In another aspect, the present disclosure features an antibody molecule, e.g., a monoclonal antibody molecule, or fragment thereof that binds TRBC1.

In some embodiments, the antibody molecule, or fragment thereof, that binds to TRBC1 comprises one or more CDRs (e.g., VHCDR1, VHCDR2, VHCDR3, VLCDR1, VLCDR2, and/or VLCDR3) disclosed in Table 2A or Table 2B, Table 3A or Table 3B, or Table 4, or a sequence having at least 85%, 90%, 95%, or 99% identity thereto. In some embodiments, the antibody molecule, or fragment thereof, that binds to TRBC1 comprises one or more framework regions (e.g., VHFWR1, VHFWR2, VHFWR3, VHFWR4, VLFWR1, VLFWR2, VLFWR3, and/or VLFWR4) disclosed in Table 2A or Table 2B, Table 3A or Table 3B, or Table 4, or a sequence having at least 85%, 90%, 95%, or 99% identity thereto. In some embodiments, the antibody molecule, or fragment thereof, that binds to TRBC1 comprises a VH and/or a VL disclosed in Table 7, or a sequence having at least 85%, 90%, 95%, or 99% identity thereto. In some embodiments, the antibody molecule, or fragment thereof, that binds to TRBC1 comprises an amino acid sequence disclosed in Table 8, or a sequence having at least 85%, 90%, 95%, or 99% identity thereto.

In some embodiments, the antibody molecule, or fragment thereof, that binds to TRBC1 comprises a VH comprising a heavy chain complementarity determining region 1 (VHCDR1), a VHCDR2, and a VHCDR3, and a VL comprising a light chain complementarity determining region 1 (VLCDR1), a VLCDR2, and a VLCDR3.

In some embodiments, the VHCDR1, VHCDR2, and VHCDR3 comprise the amino acid sequences of SEQ ID NOs: 8643, 7355, and 202, respectively (or a sequence having at least 85%, 90%, 95%, or 99% identity thereto). In some embodiments, the VHCDR1, VHCDR2, and VHCDR3 comprise the amino acid sequences of SEQ ID NOs: 8643, 201, and 202, respectively (or a sequence having at least 85%, 90%, 95%, or 99% identity thereto). In some embodiments, the VHCDR1, VHCDR2, and VHCDR3 comprise the amino acid sequences of SEQ ID NOs: 7354, 201, and 202, respectively (or a sequence having at least 85%, 90%, 95%, or 99% identity thereto). In some embodiments, the VHCDR1, VHCDR2, and VHCDR3 comprise the amino acid sequences of SEQ ID NOs: 7354, 7355, and 202, respectively (or a sequence having at least 85%, 90%, 95%, or 99% identity thereto).

In some embodiments, the VLCDR1, VLCDR2, and VLCDR3 comprise the amino acid sequences of SEQ ID NOs: 223, 224, and 225 or 8680, respectively (or a sequence having at least 85%, 90%, 95%, or 99% identity thereto). In some embodiments, the VLCDR1, VLCDR2, and VLCDR3 comprise the amino acid sequences of SEQ ID NOs: 7367, 224, and 225 or 8680, respectively (or a sequence having at least 85%, 90%, 95%, or 99% identity thereto). In some embodiments, the VLCDR1, VLCDR2, and VLCDR3 comprise the amino acid sequences of SEQ ID NOs: 223, 7368, and 225 or 8680, respectively (or a sequence having at least 85%, 90%, 95%, or 99% identity thereto). In some embodiments, the VLCDR1, VLCDR2, and VLCDR3 comprise the amino acid sequences of SEQ ID NOs: 223, 224, and 7369, respectively (or a sequence having at least 85%, 90%, 95%, or 99% identity thereto). In some embodiments, the VLCDR1, VLCDR2, and VLCDR3 comprise the amino acid sequences of SEQ ID NOs: 7367, 7368, and 7369, respectively (or a sequence having at least 85%, 90%, 95%, or 99% identity thereto).

In some embodiments, the VHCDR1, VHCDR2, VHCDR3, VLCDR1, VLCDR2, and VLCDR3 comprise the amino acid sequences of SEQ ID NOs: 8643, 7355, 202, 223, 224, and 225 or 8680, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 8643, 201, 202, 223, 224, and 225 or 8680, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of: SEQ ID NOs: 8643, 7355, 202, 7367, 224, and 225 or 8680, respectively (or a sequence having at least 85%, 90%, 95%, or 99% identity thereto); SEQ ID NOs: 8643, 7355, 202, 223, 7368, and 225 or 8680, respectively (or a sequence having at least 85%, 90%, 95%, or 99% identity thereto); SEQ ID NOs: 8643, 7355, 202, 223, 224, and 7369, respectively (or a sequence having at least 85%, 90%, 95%, or 99% identity thereto); SEQ ID NOs: 8643, 7355, 202, 7367, 7368, and 7369, respectively (or a sequence having at least 85%, 90%, 95%, or 99% identity thereto); SEQ ID NOs: 8643, 201, 202, 7367, 224, and 225 or 8680, respectively (or a sequence having at least 85%, 90%, 95%, or 99% identity thereto); SEQ ID NOs: 8643, 201, 202, 223, 7368, and 225 or 8680, respectively (or a sequence having at least 85%, 90%, 95%, or 99% identity thereto); SEQ ID NOs: 8643, 201, 202, 223, 224, and 7369, respectively (or a sequence having at least 85%, 90%, 95%, or 99% identity thereto); SEQ ID NOs: 8643, 201, 202, 7367, 7368, and 7369, respectively (or a sequence having at least 85%, 90%, 95%, or 99% identity thereto); SEQ ID NOs: 7354, 201, 202, 223, 224, and 225 or 8680, respectively (or a sequence having at least 85%, 90%, 95%, or 99% identity thereto); SEQ ID NOs: 7354, 201, 202, 7367, 224, and 225 or 8680, respectively (or a sequence having at least 85%, 90%, 95%, or 99% identity thereto); SEQ ID NOs: 7354, 201, 202, 223, 7368, and 225 or 8680, respectively (or a sequence having at least 85%, 90%, 95%, or 99% identity thereto); SEQ ID NOs: 7354, 201, 202, 223, 224, and 7369, respectively (or a sequence having at least 85%, 90%, 95%, or 99% identity thereto); SEQ ID NOs: 7354, 201, 202, 7367, 7368, and 7369, respectively (or a sequence having at least 85%, 90%, 95%, or 99% identity thereto); SEQ ID NOs: 7354, 7355, 202, 223, 224, and 225 or 8680, respectively (or a sequence having at least 85%, 90%, 95%, or 99% identity thereto); SEQ ID NOs: 7354, 7355, 202, 7367, 224, and 225 or 8680, respectively (or a sequence having at least 85%, 90%, 95%, or 99% identity thereto); SEQ ID NOs: 7354, 7355, 202, 223, 7368, and 225 or 8680, respectively (or a sequence having at least 85%, 90%, 95%, or 99% identity thereto); SEQ ID NOs: 7354, 7355, 202, 223, 224, and 7369, respectively (or a sequence having at least 85%, 90%, 95%, or 99% identity thereto); or SEQ ID NOs: 7354, 7355, 202, 7367, 7368, and 7369, respectively (or a sequence having at least 85%, 90%, 95%, or 99% identity thereto).

In some embodiments, the VH comprises an amino acid sequence selected from the group consisting of SEQ ID NOs: 7351, 253, 250-252, 254, 7343, 7344, 7350, and 7352 (or a sequence having at least 85%, 90%, 95%, or 99% identity thereto) and/or the VL comprises an amino acid sequence selected from the group consisting of SEQ ID NOs: 258, 255-257, 259, 260, and 7357-7360 (or a sequence having at least 85%, 90%, 95%, or 99% identity thereto). In some embodiments, the VH and VL comprise the amino acid sequences of SEQ ID NOs: 7351 and 258, respectively (or a sequence having at least 85%, 90%, 95%, or 99% identity thereto). In some embodiments, the VH and VL comprise the amino acid sequences of SEQ ID NOs: 253 and 258, respectively (or a sequence having at least 85%, 90%, 95%, or 99% identity thereto).

In some embodiments, the antibody molecule or fragment thereof comprises:
a heavy chain variable region (VH) comprising a heavy chain framework region 1 (VHFWR1) amino acid sequence of SEQ ID NO: 7345 (or a sequence with no more than 1, 2, 3, 4, 5, or 6 mutations, e.g., substitutions, additions, or deletions, therefrom), a VHFWR2 amino acid sequence of SEQ ID NO: 208 (or a sequence with no more than 1, 2, 3, 4, 5, or 6 mutations, e.g., substitutions, additions, or deletions, therefrom), a VHFWR3 amino acid sequence of SEQ ID NO: 217 (or a sequence with no more than 1, 2, 3, 4, 5, or 6 mutations, e.g., substitutions, additions, or deletions, therefrom), and/or a VHFWR4 amino acid sequence of SEQ ID NO: 210 (or a sequence with no more than 1, 2, 3, 4, 5, or 6 mutations, e.g., substitutions, additions, or deletions, therefrom), and
a light chain variable region (VL) comprising a light chain framework region 1 (VLFWR1) amino acid sequence of SEQ ID NO: 238 (or a sequence with no more than 1, 2, 3, 4, 5, or 6 mutations, e.g., substitutions, additions, or deletions, therefrom), a VLFWR2 amino acid sequence of SEQ ID NO: 239 (or a sequence with no more than 1, 2, 3, 4, 5, or 6 mutations, e.g., substitutions, additions, or deletions, therefrom), a VLFWR3 amino acid sequence of SEQ ID NO: 240 (or a sequence with no more than 1, 2, 3, 4, 5, or 6 mutations, e.g., substitutions, additions, or deletions, therefrom), and/or a VLFWR4 amino acid sequence of SEQ ID NO: 241 (or a sequence with no more than 1, 2, 3, 4, 5, or 6 mutations, e.g., substitutions, additions, or deletions, therefrom).

In some embodiments, the antibody molecule or fragment thereof comprises a VH comprising a VHCDR1 amino acid sequence of SEQ ID NO: 8643, a VHCDR2 amino acid sequence of SEQ ID NO: 201, and/or a VHCDR3 amino acid sequence of SEQ ID NO: 202.

In some embodiments, the antibody molecule or fragment thereof comprises a VL comprising a VLCDR1 amino acid sequence of SEQ ID NO: 223, a VLCDR2 amino acid sequence of SEQ ID NO: 224, and a VLCDR3 amino acid sequence of SEQ ID NO: 225 or 8680.

In some embodiments, the antibody molecule or fragment thereof comprises a VH comprising the amino acid sequence of SEQ ID NO: 253 (or an amino acid sequence having at least about 75%, 80%, 85%, 90%, 95%, or 99% sequence identity thereto), and/or a VL comprising the amino acid sequence of SEQ ID NO: 258 (or an amino acid sequence having at least about 93%, 95%, or 99% sequence identity thereto). In some embodiments, the antibody molecule or fragment thereof comprises a VH and/or VL substantially homologous to SEQ ID NOs: 253 and/or 258.

### Antibody Molecules Targeting TRBC2

In another aspect, the present disclosure features an antibody molecule, e.g., a monoclonal antibody molecule, or fragment thereof that binds TRBC2.

In some embodiments, the antibody molecule, or fragment thereof, that binds to TRBC2 comprises one or more CDRs (e.g., VHCDR1, VHCDR2, VHCDR3, VLCDR1, VLCDR2, and/or VLCDR3) disclosed in Table 9A, Table 9B or Table 10, or a sequence having at least 85%, 90%, 95%, or 99% identity thereto. In some embodiments, the antibody molecule, or fragment thereof, that binds to TRBC2 comprises one or more framework regions (e.g., VHFWR1, VHFWR2, VHFWR3, VHFWR4, VLFWR1, VLFWR2, VLFWR3, and/or VLFWR4) disclosed in Table 9A, Table 9B or Table 10, or a sequence having at least 85%, 90%, 95%, or 99% identity thereto. In some embodiments, the antibody molecule, or fragment thereof, that binds to TRBC2 comprises a VH and/or a VL disclosed in Table 11, or a sequence having at least 85%, 90%, 95%, or 99% identity thereto. In some embodiments, the antibody molecule, or fragment thereof, that binds to TRBC2 comprises an amino acid sequence disclosed in Table 12, or a sequence having at least 85%, 90%, 95%, or 99% identity thereto.

In some embodiments, the antibody molecule, or fragment thereof, that binds to TRBC2 comprises a VH comprising a heavy chain complementarity determining region 1 (VHCDR1), a VHCDR2, and a VHCDR3, and a VL comprising a light chain complementarity determining region 1 (VLCDR1), a VLCDR2, and a VLCDR3.

In some embodiments, the VHCDR1, VHCDR2, and VHCDR3 comprise the amino acid sequences of GX1X2MH, wherein X1 is Y or F, and X2 is P, H, V, Y, K, or A and SEQ ID NOs: 201, and 7442, respectively (or a sequence having at least 85%, 90%, 95%, or 99% identity thereto). In some embodiments, the VHCDR1, VHCDR2, and VHCDR3 comprise the amino acid sequences of SEQ ID NOs: 7422, 201, and 7403, respectively (or a sequence having at least 85%, 90%, 95%, or 99% identity thereto). In some embodiments, the VHCDR1, VHCDR2, and VHCDR3 comprise the amino acid sequences of SEQ ID NOs: 7401, 201, and 7403, respectively (or a sequence having at least 85%, 90%, 95%, or 99% identity thereto). In some embodiments, the VHCDR1, VHCDR2, and VHCDR3 comprise the amino acid sequences of SEQ ID NOs: 7394, 201, and 307, respectively (or a sequence having at least 85%, 90%, 95%, or 99% identity thereto). In some embodiments, the VHCDR1, VHCDR2, and VHCDR3 comprise the amino acid sequences of SEQ ID NOs: 8643, 201, and 8695, respectively (or a sequence having at least 85%, 90%, 95%, or 99% identity thereto). In some embodiments, the VHCDR1, VHCDR2, and VHCDR3 comprise the amino acid sequences of SEQ ID NOs: 8643, 201, and 7400, respectively (or a sequence having at least 85%, 90%, 95%, or 99% identity thereto). In some embodiments, the VHCDR1, VHCDR2, and VHCDR3 comprise the amino acid sequences of SEQ ID NOs: 7405, 201, and 7403, respectively (or a sequence having at least 85%, 90%, 95%, or 99% identity thereto). In some embodiments, the VHCDR1, VHCDR2, and VHCDR3 comprise the amino acid sequences of SEQ ID NOs: 7407, 201, and 7403, respectively (or a sequence having at least 85%, 90%, 95%, or 99% identity thereto). In some embodiments, the VHCDR1, VHCDR2, and VHCDR3 comprise the amino acid sequences of SEQ ID NOs: 7427, 201, and 7403, respectively (or a sequence having at least 85%, 90%, 95%, or 99% identity thereto). In some embodiments, the VHCDR1, VHCDR2, and VHCDR3 comprise the amino acid sequences of SEQ ID NOs: 7430, 201, and 7403, respectively (or a sequence having at least 85%, 90%, 95%, or 99% identity thereto).

In some embodiments, the VLCDR1, VLCDR2, and VLCDR3 comprise the amino acid sequences of SEQ ID NOs: 7443, 224, and 225 or 8680, respectively (or a sequence having at least 85%, 90%, 95%, or 99% identity thereto). In some embodiments, the VLCDR1, VLCDR2, and VLCDR3 comprise the amino acid sequences of SEQ ID NOs: 7410, 224, and 225 or 8680, respectively (or a sequence having at least 85%, 90%, 95%, or 99% identity thereto). In some embodiments, the VLCDR1, VLCDR2, and VLCDR3 comprise the amino acid sequences of SEQ ID NOs: 7409, 224, and 225 or 8680, respectively (or a sequence having at least 85%, 90%, 95%, or 99% identity thereto).

In some embodiments, the VHCDR1, VHCDR2, VHCDR3, VLCDR1, VLCDR2, and VLCDR3 comprise the amino acid sequences of GX1X2MH, wherein X1 is Y or F, and X2 is P, H, V, Y, K, or A and SEQ ID NOs: 201, 7442, 7443, 224, and 225 or 8680, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7422, 201, 7403, 7410, 224, and 225 or 8680, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7401, 201, 7403, 7410, 224, and 225 or 8680, respectively (or a sequence having at least 85%, 90%, 95%, or 99% identity thereto). In some embodiments, the VHCDR1, VHCDR2, VHCDR3, VLCDR1, VLCDR2, and VLCDR3 comprise the amino acid sequences of: SEQ ID NOs: 7394, 201, 307, 7410, 224, and 225 or 8680, respectively (or a sequence having at least 85%, 90%, 95%, or 99% identity thereto); SEQ ID NOs: 8643, 201, 8695, 7410, 224, and 225 or 8680, respectively (or a sequence having at least 85%, 90%, 95%, or 99% identity thereto); SEQ ID NOs: 8643, 201, 7400, 7410, 224, and 225 or 8680, respectively (or a sequence having at least 85%, 90%, 95%, or 99% identity thereto); SEQ ID NOs: 7405, 201, 7403, 7410, 224, and 225 or 8680, respectively (or a sequence having at least 85%, 90%, 95%, or 99% identity thereto); SEQ ID NOs: 7407, 201, 7403, 7410, 224, and 225 or 8680, respectively (or a sequence having at least 85%, 90%, 95%, or 99% identity thereto); SEQ ID NOs: 7427, 201, 7403, 7410, 224, and 225 or 8680, respectively (or a sequence having at least 85%, 90%, 95%, or 99% identity thereto); SEQ ID NOs: 7430, 201, 7403, 7410, 224, and 225 or 8680, respectively (or a sequence having at least 85%, 90%, 95%, or 99% identity thereto); SEQ ID NOs: 7422, 201, 7403, 7409, 224, and 225 or 8680, respectively (or a sequence having at least 85%, 90%, 95%, or 99% identity thereto); SEQ ID NOs: 7401, 201, 7403, 7409, 224, and 225 or 8680, respectively (or a sequence having at least 85%, 90%, 95%, or 99% identity thereto); SEQ ID NOs: 7394, 201, 307, 7409, 224, and 225 or 8680, respectively (or a sequence having at least 85%, 90%, 95%, or 99% identity thereto); SEQ ID NOs: 8643, 201, 8695, 7409, 224, and 225 or 8680, respectively (or a sequence having at least 85%, 90%, 95%, or 99% identity thereto); SEQ ID NOs: 8643, 201, 7400, 7409, 224, and 225 or 8680, respectively (or a sequence having at least 85%, 90%, 95%, or 99% identity thereto); SEQ ID NOs: 7405, 201, 7403, 7409, 224, and 225 or 8680, respectively (or a sequence having at least 85%, 90%, 95%, or 99% identity thereto); SEQ ID NOs: 7407, 201, 7403, 7409, 224, and 225 or 8680, respectively (or a sequence having at least 85%, 90%, 95%, or 99% identity thereto); SEQ ID NOs: 7427, 201, 7403, 7409, 224, and 225 or 8680, respectively (or a sequence having at least 85%, 90%, 95%, or 99% identity thereto); or SEQ ID NOs: 7430, 201, 7403, 7409, 224, and 225 or 8680, respectively (or a sequence having at least 85%, 90%, 95%, or 99% identity thereto).

In some embodiments, the VH comprises an amino acid sequence selected from the group consisting of SEQ ID NOs: 7420, 7423, 7411, 7412, 7413, 7414, 7415, 7416, 7417, 7425, 7428, and 7431 (or a sequence having at least 85%, 90%, 95%, or 99% identity thereto) and/or the VL comprises an amino acid sequence selected from the group consisting of SEQ ID NOs: 7419 and 7418 (or a sequence having at least 85%, 90%, 95%, or 99% identity thereto). In some embodiments, the VH and VL comprise the amino acid sequences of SEQ ID NOs: 7420 and 7419, respectively (or a sequence having at least 85%, 90%, 95%, or 99% identity thereto). In some embodiments, the VH and VL comprise the amino acid sequences of SEQ ID NOs: 7423 and 7419, respectively (or a sequence having at least 85%, 90%, 95%, or 99% identity thereto). In some embodiments, the VH and VL comprise the amino acid sequences of SEQ ID NOs: 7411 and 7419, respectively (or a sequence having at least 85%, 90%, 95%, or 99% identity thereto). In some embodiments, the VH and VL comprise the amino acid sequences of SEQ ID NOs: 7412 and 7419, respectively (or a sequence having at least 85%, 90%, 95%, or 99% identity thereto). In some embodiments, the VH and VL comprise the amino acid sequences of SEQ ID NOs: 7413 and 7419, respectively (or a sequence having at least 85%, 90%, 95%, or 99% identity thereto). In some embodiments, the VH and VL comprise the amino acid sequences of SEQ ID NOs: 7414 and 7419, respectively (or a sequence having at least 85%, 90%, 95%, or 99% identity thereto). In some embodiments, the VH and VL comprise the amino acid sequences of SEQ ID NOs: 7415 and 7419, respectively (or a sequence having at least 85%, 90%, 95%, or 99% identity thereto). In some embodiments, the VH and VL comprise the amino acid sequences of SEQ ID NOs: 7416 and 7419, respectively (or a sequence having at least 85%, 90%, 95%, or 99% identity thereto). In some embodiments, the VH and VL comprise the amino acid sequences of SEQ ID NOs: 7417 and 7419, respectively (or a sequence having at least 85%, 90%, 95%, or 99% identity thereto). In some embodiments, the VH and VL comprise the amino acid sequences of SEQ ID NOs: 7425 and 7419, respectively (or a sequence having at least 85%, 90%, 95%, or 99% identity thereto). In some embodiments, the VH and VL comprise the amino acid sequences of SEQ ID NOs: 7428 and 7419, respectively (or a sequence having at least 85%, 90%, 95%, or 99% identity thereto). In some embodiments, the VH and VL comprise the amino acid sequences of SEQ ID NOs: 7431 and 7419, respectively (or a sequence having at least 85%, 90%, 95%, or 99% identity thereto). In some embodiments, the VH and VL comprise the amino acid sequences of SEQ ID NOs: 7420 and 7418, respectively (or a sequence having at least 85%, 90%, 95%, or 99% identity thereto). In some embodiments, the VH and VL comprise the amino acid sequences of SEQ ID NOs: 7423 and 7418, respectively (or a sequence having at least 85%, 90%, 95%, or 99% identity thereto). In some embodiments, the VH and VL comprise the amino acid sequences of SEQ ID NOs: 7411 and 7418, respectively (or a sequence having at least 85%, 90%, 95%, or 99% identity thereto). In some embodiments, the VH and VL comprise the amino acid sequences of SEQ ID NOs: 7412 and 7418, respectively (or a sequence having at least 85%, 90%, 95%, or 99% identity thereto). In some embodiments, the VH and VL comprise the amino acid sequences of SEQ ID NOs: 7413 and 7418, respectively (or a sequence having at least 85%, 90%, 95%, or 99% identity thereto). In some embodiments, the VH and VL comprise the amino acid sequences of SEQ ID NOs: 7414 and 7418, respectively (or a sequence having at least 85%, 90%, 95%, or 99% identity thereto). In some embodiments, the VH and VL comprise the amino acid sequences of SEQ ID NOs: 7415 and 7418, respectively (or a sequence having at least 85%, 90%, 95%, or 99% identity thereto). In some embodiments, the VH and VL comprise the amino acid sequences of SEQ ID NOs: 7416 and 7418, respectively (or a sequence having at least 85%, 90%, 95%, or 99% identity thereto). In some embodiments, the VH and VL comprise the amino acid sequences of SEQ ID NOs: 7417 and 7418, respectively (or a sequence having at least 85%, 90%, 95%, or 99% identity thereto). In some embodiments, the VH and VL comprise the amino acid sequences of SEQ ID NOs: 7425 and 7418, respectively (or a sequence having at least 85%, 90%, 95%, or 99% identity thereto). In some embodiments, the VH and VL comprise the amino acid sequences of SEQ ID NOs: 7428 and 7418, respectively (or a sequence having at least 85%, 90%, 95%, or 99% identity thereto). In some embodiments, the VH and VL comprise the amino acid sequences of SEQ ID NOs: 7431 and 7418, respectively (or a sequence having at least 85%, 90%, 95%, or 99% identity thereto).

In another aspect, the disclosure features an antibody molecule, e.g., an IgM antibody molecule comprising: (i) a first antigen binding domain that selectively binds to T cell receptor beta chain constant domain 1 (TRBC1) or T cell receptor beta chain constant domain 2 (TRBC2), and (ii) a complement activating domain that activates the complement pathway, e.g., by binding C1q. In some embodiments, an antibody molecule, e.g., IgM antibody molecule, comprises an antigen binding domain that targets TRBC1. In some embodiments, the antibody molecule is an IgM antibody molecule, e.g., that multimerizes into tetramers, pentamers, and/or hexamers and is capable of activating complement pathway(s). In some embodiments, the IgM antibody molecule comprises an antigen binding domain that targets TRBC1 comprising a heavy chain comprising the amino acid sequence of SEQ ID NO: 6173 (or an amino acid sequence having at least about 93%, 95%, or 99% sequence identity to SEQ ID NO: 6173).

(SEQ ID NO: 6173)
METDTLLLWVLLLWVPGSTGQVQLVQSGAEVKKPGSSVKVSCKASGYTFT
GYVMHWVRQAPGQGLEWMGFINPYNDDIQSNERFRGRVTITSDKSTTTAY
MELSSLRSEDTAVYYCARGAGYNFDGAYRFFDFWGQGTLVTVSSGSASAP
TLFPLVSCENSPSDTSSVAVGCLAQDFLPDSITFSWKYKNNSDISSTRGF
PSVLRGGKYAATSQVLLPSKDVMQGTDEHVVCKVQHPNGNKEKNVPLPVI
AELPPKVSVFVPPRDGFFGNPRKSKLICQATGFSPRQIQVSWLREGKQVG
SGVTTDQVQAEAKESGPTTYKVTSTLTIKESDWLGQSMFTCRVDHRGLTF
QQNASSMCVPDQDTAIRVFAIPPSFASIFLTKSTKLTCLVTDLTTYDSVT
ISWTRQNGEAVKTHTNISESHPNATFSAVGEASICEDDWNSGERFTCTVT
HTDLPSPLKQTISRPKGVALHRPDVYLLPPAREQLNLRESATITCLVTGF
SPADVFVQWMQRGQPLSPEKYVTSAPMPEPQAPGRYFAHSILTVSEEEWN
TGETYTCVVAHEALPNRVTERTVDKSTGKPTLYNVSLVMSDTAGTCY.

In some embodiments, the IgM antibody molecule comprises an antigen binding domain that targets TRBC1 comprising a light chain comprising the amino acid sequence of SEQ ID NO: 6174 (or an amino acid sequence having at least about 93%, 95%, or 99% sequence identity to SEQ ID NO: 6174).

(SEQ ID NO: 6174)
MKNHLLFWGVLAVFIKAVHVKAQEDERIVLVDNKCKCARITSRIIRSSED
PNEDIVERNIRIIVPLNNRENISDPTSPLRTRFVYHLSDLCKKCDPTEVE
LDNQIVTATQSNICDEDSATETCYTYDRNKCYTAVVPLVYGGETKMVETA
LTPDACYPD.

In some embodiments, the IgM antibody molecule comprises an antigen binding domain that targets TRBC1 comprising amino acid sequences of SEQ ID NO: 6173 and 6174 (or amino acid sequences having at least about 93%, 95%, or 99% sequence identity to SEQ ID NO: 6173 and 6174) and an amino acid sequence of a light chain sequence provided herein, e.g., in Table 4 or Table 7.

In some embodiments, the complement activating domain comprises a portion of an antibody molecule capable of binding or being bound by C1q, e.g., a portion of a IgG1, IgG2, IgG3, IgG4, IgM, IgAQ1, IgA2, IgD, or IgE. In some embodiments, a complement activating domain comprises a Ch2, Ch3, or Ch4 domain.

Without wishing to be bound by theory, it is thought that complement activation in proximity to a target cell (e.g., a TRBC1 or TRBC2 expressing cell, e.g., a lymphocyte expressing TRBC1 or TRBC2, e.g., a lymphoma cell expressing TRBC1 or TRBC2) may induce the death of the target cell. In some embodiments, use of an antibody molecule, e.g., IgM antibody molecule, or a multifunctional molecule in the methods described herein induces complement mediated cell death of the target cell.

In another aspect, the disclosure features a multispecific antibody molecule (e.g., a bispecific antibody molecule) that binds to TRBC1 and NKp30. In some embodiments, the multispecific antibody molecule comprises one or more moieties that bind to TRBC1, e.g., one or more Fabs that bind to TRBC1, e.g., one or two Fabs that bind to TRBC1. In some embodiments, the multispecific antibody molecule comprises one or more moieties that bind to NKp30, e.g., one or more scFvs that bind to NKp30, e.g., one or two scFvs that bind to NKp30. In some embodiments, the moiety that binds to TRBC1 comprises an anti-TRBC1 sequence disclosed herein, e.g., comprises a CDR, VH, VL, heavy chain, or light chain sequence disclosed in Table 1, Table 2A or Table 2B, Table 4, Table 7, Table 8, or a sequence having at least 70, 80, 90, 95, or 99% identity thereto. In some embodiments, the moiety that binds to NKp30 comprises an anti-NKp30 sequence disclosed herein, e.g., comprises a CDR, VH, VL, heavy chain, or light chain sequence disclosed in Table 20A or Table 20B, Table 22, Table 23A or Table 23B, Table 24, Table 25, Table 26, and Table 21A or Table 21B, or a sequence having at least 70, 80, 90, 95, or 99% identity thereto.

In some embodiments, the multispecific antibody molecule comprises an anti-TRBC1 antibody molecule and an anti-NKp30 antibody molecule, e.g., an anti-TRBC1 antibody molecule comprising two heavy chains and two light chains, and an anti-NKp30 scFv that is fused to the N-terminus of one of the heavy chains of the anti-TRBC1 antibody. In some embodiments, the two heavy chains of the anti-TRBC1 antibody form a heterodimer, e.g., via knob-and-hole mutations. In some embodiments, the two heavy chains of the anti-TRBC1 antibody comprise the N297A mutation. In some embodiments, the two heavy chains of the anti-TRBC1 antibody do not comprise the N297A mutation. In some embodiments, the multispecific antibody molecule comprises a first chain, a second chain, a third chain, and a fourth chain, wherein the first chain comprises an anti-TRBC1 light chain variable region (VL) and a light chain constant region (CL); the second chain comprises an anti-NKp30 scFv, an anti-TRBC1 heavy chain variable region (VH), a CH1, a CH2, and a CH3; the third chain comprises an anti-TRBC1 VH, a CH1, a CH2, and a CH3; and the fourth chain comprises an anti-TRBC1 VL and a CL.

In some embodiments, the multispecific antibody molecule comprises an anti-TRBC1 antibody molecule and an anti-NKp30 antibody molecule. In some embodiments, the multispecific antibody molecule comprises an anti-TRBC1 Fab, an anti-NKp30 scFv, and an Fe dimer comprising two Fc chains. In some embodiments, the C-terminus of the heavy chain of the anti-TRBC1 Fab is fused to the N-terminus of one Fc chain, and the anti-NKp30 scFv is fused to the N-terminus of the other Fc chain. In some embodiments, the two Fc chains form a heterodimer, e.g., via knob-and-hole mutations. In some embodiments, the two Fc chains comprise the N297A mutation. In some embodiments, the two Fc chains do not comprise the N297A mutation. In some embodiments, the multispecific antibody molecule comprises a first chain, a second chain, and a third chain, wherein the first chain comprises an anti-TRBC1 VL and a CL; the second chain comprises an anti-TRBC1 VH, a CH1, a CH2, and a CH3; and the third chain comprises an anti-NKp30 scFv, a CH2, and a CH3.

In some embodiments, the multispecific antibody molecule comprises an anti-TRBC1 antibody molecule and an anti-NKp30 antibody molecule, e.g., an anti-TRBC1 antibody molecule comprising two heavy chains and two light chains, and two anti-NKp30 scFvs that are fused to the C-terminus of the two light chains of the anti-TRBC1 antibody molecule, respectively. In some embodiments, the two heavy chains of the anti-TRBC1 antibody form a homodimer. In some embodiments, the two heavy chains of the anti-TRBC1 antibody comprise the N297A mutation. In some embodiments, the two heavy chains of the anti-TRBC1 antibody do not comprise the N297A mutation. In some embodiments, the multispecific antibody molecule comprises a first chain, a second chain, a third chain, and a fourth chain, wherein the first chain comprises an anti-TRBC1 VL, a CL, and an anti-NKp30 scFv; the second chain comprises an anti-TRBC1 VH, a CH1, a CH2, and a CH3; the third chain comprises an anti-TRBC1 VH, a CH1, a CH2, and a CH3; and the fourth chain comprises an anti-TRBC1 VL, a CL, and an anti-NKp30 scFv.

In some embodiments, the multispecific antibody molecule comprises an anti-TRBC1 antibody molecule and an anti-NKp30 antibody molecule, e.g., an anti-TRBC1 antibody molecule comprising two heavy chains and two light chains, and two anti-NKp30 scFvs that are fused to the N-terminus of the two heavy chains of the anti-TRBC1 antibody molecule, respectively. In some embodiments, the two heavy chains of the anti-TRBC1 antibody form a homodimer. In some embodiments, the two heavy chains of the anti-TRBC1 antibody comprise the N297A mutation. In some embodiments, the two heavy chains of the anti-TRBC1 antibody do not comprise the N297A mutation. In some embodiments, the multispecific antibody molecule comprises a first chain, a second chain, a third chain, and a fourth chain, wherein the first chain comprises an anti-TRBC1 VL and a CL; the second chain comprises an anti-NKp30 scFv, an anti-TRBC1 VH, a CH1, a CH2, and a CH3; the third chain comprises an anti-NKp30 scFv, an anti-TRBC1 VH, a CH1, a CH2, and a CH3; and the fourth chain comprises an anti-TRBC1 VL and a CL.

Uses of the antibody molecules disclosed herein include but are not limited to methods of treating cancer (e.g., a cancer expressing TRBC1) disclosed herein; methods of identifying, evaluating, or selecting a subject in need of treatment (e.g., determining whether a subject has cancer cells that express TRBC1) disclosed herein; and methods of laboratory or diagnostic analysis (e.g., immunological assays comprising detecting the presence and/or level of TRBC1 or TRBC1 expressing cells).

### Cytokine Molecules and Cytokine Inhibitor Molecules

In some embodiments, the multifunctional molecule includes a cytokine molecule. As used herein, a “cytokine molecule” or a “cytokine polypeptide” as interchangeably used herein, refers to full length, a fragment or a variant of a cytokine; a cytokine further comprising a receptor domain, e.g., a cytokine receptor dimerizing domain; or an agonist of a cytokine receptor, e.g., an antibody molecule (e.g., an agonistic antibody) to a cytokine receptor, that elicits at least one activity of a naturally-occurring cytokine. In some embodiments, a cytokine molecule refers to a cytokine polypeptide or a functional fragment or variant thereof. In some embodiments the cytokine molecule is chosen from interleukin-2 (IL-2), interleukin-7 (IL-7), interleukin-12 (IL-12), interleukin-10 (IL-10), interleukin-15 (IL-15), interleukin-18 (IL-18), interleukin-21 (IL-21), or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain. In other embodiments, the cytokine molecule is an agonist of a cytokine receptor, e.g., an antibody molecule (e.g., an agonistic antibody) to a cytokine receptor chosen from an IL-15Ra or IL-21R.

Cytokines are generally polypeptides that influence cellular activity, for example, through signal transduction pathways. Accordingly, a cytokine of the multispecific or multifunctional polypeptide is useful and can be associated with receptor-mediated signaling that transmits a signal from outside the cell membrane to modulate a response within the cell. Cytokines are proteinaceous signaling compounds that are mediators of the immune response. They control many different cellular functions including proliferation, differentiation and cell survival/apoptosis; cytokines are also involved in several pathophysiological processes including viral infections and autoimmune diseases. Cytokines are synthesized under various stimuli by a variety of cells of both the innate (monocytes, macrophages, dendritic cells) and adaptive (T- and B-cells) immune systems. Cytokines can be classified into two groups: pro- and anti-inflammatory. Pro-inflammatory cytokines, including IFNγ, IL-1, IL-6 and TNF-alpha, are predominantly derived from the innate immune cells and Th1 cells. Anti-inflammatory cytokines, including IL-10, IL-4, IL-13 and IL-5, are synthesized from Th2 immune cells.

Provided herein are, inter alia, multispecific (e.g., bi-, tri-, quad-specific) or multifunctional molecules, that include, e.g., are engineered to contain, one or more cytokine molecules, e.g., immunomodulatory (e.g., proinflammatory) cytokines and variants, e.g., functional variants, thereof. Accordingly, in some embodiments, the cytokine molecule is an interleukin or a variant, e.g., a functional variant thereof. In some embodiments the interleukin is a proinflammatory interleukin. In some embodiments the interleukin is chosen from interleukin-2 (IL-2), interleukin-12 (IL-12), interleukin-15 (IL-15), interleukin-18 (IL-18), interleukin-21 (IL-21), interleukin-7 (IL-7), or interferon gamma. In some embodiments, the cytokine molecule is a proinflammatory cytokine.

In certain embodiments, the cytokine is a single chain cytokine. In certain embodiments, the cytokine is a multichain cytokine (e.g., the cytokine comprises 2 or more (e.g., 2) polypeptide chains. An exemplary multichain cytokine is IL-12.

Examples of useful cytokines include, but are not limited to, GM-CSF, IL-1α, IL-1β, IL-2, IL-3, IL-4, IL-5, IL-6, IL-7, IL-8, IL-10, IL-12, IL-21, IFN-α, IFN-β, IFN-γ, MIP-1α, MIP-1β, TGF-β, TNF-α, and TNFβ. In some embodiments the cytokine of the multispecific or multifunctional polypeptide is a cytokine selected from the group of GM-CSF, IL-2, IL-7, IL-8, IL-10, IL-12, IL-15, IL-21, IFN-α, IFN-γ, MIP-1α, MIP-1β and TGF-β. In some embodiments the cytokine of the i the multispecific or multifunctional polypeptide is a cytokine selected from the group of IL-2, IL-7, IL-10, IL-12, IL-15, IFN-α, and IFN-γ. In certain embodiments the cytokine is mutated to remove N- and/or O-glycosylation sites. Elimination of glycosylation increases homogeneity of the product obtainable in recombinant production.

In some embodiments, the cytokine of the multispecific or multifunctional polypeptide is IL-2. In a specific embodiment, the IL-2 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity. In another particular embodiment the IL-2 cytokine is a mutant IL-2 cytokine having reduced binding affinity to the .alpha.-subunit of the IL-2 receptor. Together with the .beta.- and .gamma.-subunits (also known as CD122 and CD132, respectively), the .alpha.-subunit (also known as CD25) forms the heterotrimeric high-affinity IL-2 receptor, while the dimeric receptor consisting only of the β- and γ-subunits is termed the intermediate-affinity IL-2 receptor. As described in PCT patent application number PCT/EP2012/051991, which is incorporated herein by reference in its entirety, a mutant IL-2 polypeptide with reduced binding to the .alpha.-subunit of the IL-2 receptor has a reduced ability to induce IL-2 signaling in regulatory T cells, induces less activation-induced cell death (AICD) in T cells, and has a reduced toxicity profile in vivo, compared to a wild-type IL-2 polypeptide. The use of such a cytokine with reduced toxicity is particularly advantageous in a multispecific or multifunctional polypeptide according to the invention, having a long serum half-life due to the presence of an Fc domain. In some embodiments, the mutant IL-2 cytokine of the multispecific or multifunctional polypeptide according to the invention comprises at least one amino acid mutation that reduces or abolishes the affinity of the mutant IL-2 cytokine to the .alpha.-subunit of the IL-2 receptor (CD25) but preserves the affinity of the mutant IL-2 cytokine to the intermediate-affinity IL-2 receptor (consisting of the β and γ subunits of the IL-2 receptor), compared to the non-mutated IL-2 cytokine. In some embodiments the one or more amino acid mutations are amino acid substitutions. In a specific embodiment, the mutant IL-2 cytokine comprises one, two or three amino acid substitutions at one, two or three position(s) selected from the positions corresponding to residue 42, 45, and 72 of human IL-2. In a more specific embodiment, the mutant IL-2 cytokine comprises three amino acid substitutions at the positions corresponding to residue 42, 45 and 72 of human IL-2. In an even more specific embodiment, the mutant IL-2 cytokine is human IL-2 comprising the amino acid substitutions F42A, Y45A and L72G. In some embodiments the mutant IL-2 cytokine additionally comprises an amino acid mutation at a position corresponding to position 3 of human IL-2, which eliminates the O-glycosylation site of IL-2. Particularly, said additional amino acid mutation is an amino acid substitution replacing a threonine residue by an alanine residue. A particular mutant IL-2 cytokine useful in the invention comprises four amino acid substitutions at positions corresponding to residues 3, 42, 45 and 72 of human IL-2. Specific amino acid substitutions are T3A, F42A, Y45A and L72G. As demonstrated in PCT patent application number PCT/EP2012/051991 and in the appended Examples, said quadruple mutant IL-2 polypeptide (IL-2 qm) exhibits no detectable binding to CD25, reduced ability to induce apoptosis in T cells, reduced ability to induce IL-2 signaling in T.sub.reg cells, and a reduced toxicity profile in vivo. However, it retains ability to activate IL-2 signaling in effector cells, to induce proliferation of effector cells, and to generate IFN-γ as a secondary cytokine by NK cells.

The IL-2 or mutant IL-2 cytokine according to any of the above embodiments may comprise additional mutations that provide further advantages such as increased expression or stability. For example, the cysteine at position 125 may be replaced with a neutral amino acid such as alanine, to avoid the formation of disulfide-bridged IL-2 dimers. Thus, in certain embodiments the IL-2 or mutant IL-2 cytokine of the multispecific or multifunctional polypeptide according to the invention comprises an additional amino acid mutation at a position corresponding to residue 125 of human IL-2. In some embodiments said additional amino acid mutation is the amino acid substitution C125A.

In a specific embodiment the IL-2 cytokine of the multispecific or multifunctional polypeptide comprises the polypeptide sequence of SEQ ID NO: 2270 [APTSSSTKKTQLQLEHLLLDLQMILNGINNYKNPKLTRMLTFKFYMPKKATELKHLQCLEEELK PLEEVLNLAQSKNFHLRPRDLISNINVIVLELKGSETTFMCEYADETATIVEFLNRWITFAQSIISTL T].

In another specific embodiment the IL-2 cytokine of the multispecific or multifunctional polypeptide comprises the polypeptide sequence of SEQ ID NO: 2280 [APASSSTKKTQLQLEHLLLDLQMILNGINNYKNPKLTRMLTAKFAMPKKATELKHLQCLEEEL KPLEEVLNGAQSKNFHLRPRDLISNINVIVLELKGSETTFMCEYADETATIVEFLNRWITFAQSIIS TLT].

In another embodiment the cytokine of the multispecific or multifunctional polypeptide is IL-12. In a specific embodiment said IL-12 cytokine is a single chain IL-12 cytokine. In an even more specific embodiment the single chain IL-12 cytokine comprises the polypeptide sequence of SEQ ID NO: 2290 [IWELKKDVYVVELDWYPDAPGEMVVLTCDTPEEDGITWTLDQSSEVLGSGKTLTIQVKEFGDA GQYTCHKGGEVLSHSLLLLHKKEDGIWSTDILKDQKEPKNKTFLRCEAKNYSGRFTCWWLTTIS TDLTFSVKSSRGSSDPQGVTCGAATLSAERVRGDNKEYEYSVECQEDSACPAAEESLPIEVMVD AVHKLKYENYTSSFFIRDIIKPDPPKNLQLKPLKNSRQVEVSWEYPDTWSTPHSYFSLTFCVQVQ GKSKREKKDRVFTDKTSATVICRKNASISVRAQDRYYSSSWSEWASVPCSGGGGSGGGGSGGG GSRNLPVATPDPGMFPCLHHSQNLLRAVSNMLQKARQTLEFYPCTSEEIDHEDITKDKTSTVEAC LPLELTKNESCLNSRETSFITNGSCLASRKTSFMMALCLSSIYEDLKMYQVEFKTMNAKLLMDP KRQIFLDQNMLAVIDELMQALNFNSETVPQKSSLEEPDFYKTKIKLCILLHAFRIRAVTIDRVMSY LNAS]. In some embodiments, the IL-12 cytokine can elicit one or more of the cellular responses selected from the group consisting of: proliferation in a NK cell, differentiation in a NK cell, proliferation in a T cell, and differentiation in a T cell.

In another embodiment the cytokine of the multispecific or multifunctional polypeptide is IL-10. In a specific embodiment said IL-10 cytokine is a single chain IL-10 cytokine. In an even more specific embodiment the single chain IL-10 cytokine comprises the polypeptide sequence of SEQ ID NO: 2300 [SPGQGTQSENSCTHFPGNLPNMLRDLRDAFSRVKTFFQMKDQLDNLLLKESLLEDFKGYLGCQ ALSEMIQFYLEEVMPQAENQDPDIKAHVNSLGENLKTLRLRLRRCHRFLPCENKSKAVEQVKNA FNKLQEKGIYKAMSEFDIFINYIEAYMTMKIRNGGGGSGGGGSGGGGSGGGGSSPGQGTQSENS CTHFPGNLPNMLRDLRDAFSRVKTFFQMKDQLDNLLLKESLLEDFKGYLGCQALSEMIQFYLEE VMPQAENQDPDIKAHVNSLGENLKTLRLRLRRCHRFLPCENKSKAVEQVKNAFNKLQEKGIYK AMSEFDIFINYIEAYMTMKIRN].

In another specific embodiment the IL-10 cytokine is a monomeric IL-10 cytokine. In a more specific embodiment, the monomeric IL-10 cytokine comprises the polypeptide sequence of SEQ ID NO: 2310 [SPGQGTQSENSCTHFPGNLPNMLRDLRDAFSRVKTFFQMKDQLDNLLLKESLLEDFKGYLGCQ ALSEMIQFYLEEVMPQAENQDPDIKAHVNSLGENLKTLRLRLRRCHRFLPCENGGGSGGKSKAV EQVKNAFNKLQEKGIYKAMSEFDIFINYIEAYMTMKIRN]. In some embodiments, the IL-10 cytokine can elicit one or more of the cellular responses selected from the group consisting of: inhibition of cytokine secretion, inhibition of antigen presentation by antigen presenting cells, reduction of oxygen radical release, and inhibition of T cell proliferation. A multispecific or multifunctional polypeptide according to the invention wherein the cytokine is IL-10 is particularly useful for downregulation of inflammation, e.g. in the treatment of an inflammatory disorder.

In another embodiment, the cytokine of the multispecific or multifunctional polypeptide is IL-15. In a specific embodiment said IL-15 cytokine is a mutant IL-15 cytokine having reduced binding affinity to the α-subunit of the IL-15 receptor. Without wishing to be bound by theory, a mutant IL-15 polypeptide with reduced binding to the .alpha.-subunit of the IL-15 receptor has a reduced ability to bind to fibroblasts throughout the body, resulting in improved pharmacokinetics and toxicity profile, compared to a wild-type IL-15 polypeptide. The use of an cytokine with reduced toxicity, such as the described mutant IL-2 and mutant IL-15 effector moieties, is particularly advantageous in a multispecific or multifunctional polypeptide according to the invention, having a long serum half-life due to the presence of an Fc domain. In some embodiments the mutant IL-15 cytokine of the multispecific or multifunctional polypeptide according to the invention comprises at least one amino acid mutation that reduces or abolishes the affinity of the mutant IL-15 cytokine to the .alpha.-subunit of the IL-15 receptor but preserves the affinity of the mutant IL-15 cytokine to the intermediate-affinity IL-15/IL-2 receptor (consisting of the .beta.- and .gamma.-subunits of the IL-15/IL-2 receptor), compared to the non-mutated IL-15 cytokine. In some embodiments the amino acid mutation is an amino acid substitution. In a specific embodiment, the mutant IL-15 cytokine comprises an amino acid substitution at the position corresponding to residue 53 of human IL-15. In a more specific embodiment, the mutant IL-15 cytokine is human IL-15 comprising the amino acid substitution E53A. In some embodiments the mutant IL-15 cytokine additionally comprises an amino acid mutation at a position corresponding to position 79 of human IL-15, which eliminates the N-glycosylation site of IL-15. Particularly, said additional amino acid mutation is an amino acid substitution replacing an asparagine residue by an alanine residue. In an even more specific embodiment the IL-15 cytokine comprises the polypeptide sequence of SEQ ID NO: 2320 [NWVNVISDLKKIEDLIQSMHIDATLYTESDVHPSCKVTAMKCFLLELQVISLASGDASIHDTVEN LIILANNSLSSNGAVTESGCKECEELEEKNIKEFLQSFVHIVQMFINTS]. In some embodiments, the IL-15 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity.

Mutant cytokine molecules useful as effector moieties in the multispecific or multifunctional polypeptide can be prepared by deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing. Substitution or insertion may involve natural as well as non-natural amino acid residues. Amino acid modification includes well known methods of chemical modification such as the addition or removal of glycosylation sites or carbohydrate attachments, and the like.

In some embodiments, the cytokine, particularly a single-chain cytokine, of the multispecific or multifunctional polypeptide is GM-CSF. In a specific embodiment, the GM-CSF cytokine can elicit proliferation and/or differentiation in a granulocyte, a monocyte or a dendritic cell. In some embodiments, the cytokine, particularly a single-chain cytokine, of the multispecific or multifunctional polypeptide is IFN-α. In a specific embodiment, the IFN-α cytokine can elicit one or more of the cellular responses selected from the group consisting of: inhibiting viral replication in a virus-infected cell, and upregulating the expression of major histocompatibility complex I (MHC I). In another specific embodiment, the IFN-α cytokine can inhibit proliferation in a tumor cell. In some embodiments the cytokine, particularly a single-chain cytokine, of the multispecific or multifunctional polypeptide is IFNγ. In a specific embodiment, the IFN-γ cytokine can elicit one or more of the cellular responses selected from the group of: increased macrophage activity, increased expression of MHC molecules, and increased NK cell activity. In some embodiments the cytokine, particularly a single-chain cytokine, of the multispecific or multifunctional polypeptide is IL-7. In a specific embodiment, the IL-7 cytokine can elicit proliferation of T and/or B lymphocytes. In some embodiments, the cytokine, particularly a single-chain cytokine, of the multispecific or multifunctional polypeptide is IL-8. In a specific embodiment, the IL-8 cytokine can elicit chemotaxis in neutrophils. In some embodiments, the cytokine, particularly a single-chain cytokine, of the multispecific or multifunctional polypeptide, is MIP-1α. In a specific embodiment, the MIP-1α cytokine can elicit chemotaxis in monocytes and T lymphocyte cells. In some embodiments, the cytokine, particularly a single-chain cytokine, of the multispecific or multifunctional polypeptide is MIP-1β. In a specific embodiment, the MIP-1β cytokine can elicit chemotaxis in monocytes and T lymphocyte cells. In some embodiments, the cytokine, particularly a single-chain cytokine, of the multispecific or multifunctional polypeptide is TGF-β. In a specific embodiment, the TGF-β cytokine can elicit one or more of the cellular responses selected from the group consisting of: chemotaxis in monocytes, chemotaxis in macrophages, upregulation of IL-1 expression in activated macrophages, and upregulation of IgA expression in activated B cells.

In some embodiments, the multispecific or multifunctional polypeptide of the invention binds to a cytokine receptor with a dissociation constant (K_{D}) that is at least about 1, 1.5, 2, 2.5, 3, 3.5, 4, 4.5, 5, 5.5, 6, 6.5, 7, 7.5, 8, 8.5, 9, 9.5 or 10 times greater than that for a control cytokine. In another embodiment, the multispecific or multifunctional polypeptide binds to a cytokine receptor with a K_{D} that is at least 2, 3, 4, 5, 6, 7, 8, 9, or 10 times greater than that for a corresponding multispecific or multifunctional polypeptide comprising two or more effector moieties. In another embodiment, the multispecific or multifunctional polypeptide binds to an cytokine receptor with a dissociation constant K_{D} that is about 10 times greater than that for a corresponding the multispecific or multifunctional polypeptide comprising two or more cytokines.

In some embodiments, the multispecific molecules as described herein include a cytokine molecule. In embodiments, the cytokine molecule includes a full length, a fragment or a variant of a cytokine; a cytokine receptor domain, e.g., a cytokine receptor dimerizing domain; or an agonist of a cytokine receptor, e.g., an antibody molecule (e.g., an agonistic antibody) to a cytokine receptor.

In some embodiments the cytokine molecule is chosen from IL-2, IL-12, IL-15, IL-18, IL-7, IL-21, or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain.

In other embodiments, the cytokine molecule is an agonist of a cytokine receptor, e.g., an antibody molecule (e.g., an agonistic antibody) to a cytokine receptor chosen from an IL-15Ra or IL-21R.

In some embodiments, the cytokine molecule is IL-15, e.g., human IL-15 (e.g., comprising the amino acid sequence: NWVNVISDLKKIEDLIQSMHIDATLYTESDVHPSCKVTAMKCFLLELQVISLESGDASIHDTVEN LIILANNSLSSNGNVTESGCKECEELEEKNIKEFLQSFVHIVQMFINTS (SEQ ID NO: 2170), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2170.

In some embodiments, the cytokine molecule comprises a receptor dimerizing domain, e.g., an IL15Ralpha dimerizing domain. In some embodiments, the IL15Ralpha dimerizing domain comprises the amino acid sequence: MAPRRARGCRTLGLPALLLLLLLRPPATRGITCPPPMSVEHADIWVKSYSLYSRERYICNSGFKR KAGTSSLTECVL (SEQ ID NO: 2180), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2180. In some embodiments, the cytokine molecule (e.g., IL-15) and the receptor dimerizing domain (e.g., an IL15Ralpha dimerizing domain) of the multispecific molecule are covalently linked, e.g., via a linker (e.g., a Gly-Ser linker, e.g., a linker comprising the amino acid sequence SGGSGGGGSGGGSGGGGSLQ (SEQ ID NO: 2190). In other embodiments, the cytokine molecule (e.g., IL-15) and the receptor dimerizing domain (e.g., an IL15Ralpha dimerizing domain) of the multispecific molecule are not covalently linked, e.g., are non-covalently associated.

In other embodiments, the cytokine molecule is IL-2, e.g., human IL-2 (e.g., comprising the amino acid sequence: APTSSSTKKTQLQLEHLLLDLQMILNGINNYKNPKLTRMLTFKFYMPKKATELKHLQCLEEELK PLEEVLNLAQSKNFHLRPRDLISNINVIVLELKGSETTFMCEYADETATIVEFLNRWITFCQSIISTL T (SEQ ID NO: 2191), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO:2191).

In other embodiments, the cytokine molecule is IL-18, e.g., human IL-18 (e.g., comprising the amino acid sequence: YFGKLESKLSVIRNLNDQVLFIDQGNRPLFEDMTDSDCRDNAPRTIFIISMYKDSQPRGMAVTISV KCEKISTLSCENKIISFKEMNPPDNIKDTKSDIIFFQRSVPGHDNKMQFESSSYEGYFLACEKERDL FKLILKKEDELGDRSIMFTVQNED (SEQ ID NO: 2192), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2192).

In other embodiments, the cytokine molecule is IL-21, e.g., human IL-21 (e.g., comprising the amino acid sequence: QGQDRHMIRMRQLIDIVDQLKNYVNDLVPEFLPAPEDVETNCEWSAFSCFQKAQLKSANTGNN ERIINVSIKKLKRKPPSTNAGRRQKHRLTCPSCDSYEKKPPKEFLERFKSLLQKMIHQHLSSRTHG SEDS (SEQ ID NO: 2193), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2193).

In yet other embodiments, the cytokine molecule is interferon gamma, e.g., human interferon gamma (e.g., comprising the amino acid sequence: QDPYVKEAENLKKYFNAGHSDVADNGTLFLGILKNWKEESDRKIMQSQIVSFYFKLFKNFKDD QSIQKSVETIKEDMNVKFFNSNKKKRDDFEKLTNYSVTDLNVQRKAIHELIQVMAELSPAAKTG KRKRSQMLFRG (SEQ ID NO: 2194), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2194).

### Immune Cell Engagers

The immune cell engagers of the multispecific or multifunctional molecules disclosed herein can mediate binding to, and/or activation of, an immune cell, e.g., an immune effector cell. In some embodiments, the immune cell is chosen from a T cell, an NK cell, a B cell, a dendritic cell, or a macrophage cell engager, or any combination thereof. In some embodiments, the immune cell engager is chosen from one, two, three, or all of a T cell engager, NK cell engager, a B cell engager, a dendritic cell engager, or a macrophage cell engager, or any combination thereof. The immune cell engager can be an agonist of the immune system. In some embodiments, the immune cell engager can be an antibody molecule, a ligand molecule (e.g., a ligand that further comprises an immunoglobulin constant region, e.g., an Fc region), a small molecule, a nucleotide molecule.

### Natural Killer Cell Engagers

Natural Killer (NK) cells recognize and destroy tumors and virus-infected cells in an antibody-independent manner. The regulation of NK cells is mediated by activating and inhibiting receptors on the NK cell surface. One family of activating receptors is the natural cytotoxicity receptors (NCRs) which include NKp30, NKp44 and NKp46. The NCRs initiate tumor targeting by recognition of heparan sulfate on cancer cells. NKG2D is a receptor that provides both stimulatory and costimulatory innate immune responses on activated killer (NK) cells, leading to cytotoxic activity. DNAM1 is a receptor involved in intercellular adhesion, lymphocyte signaling, cytotoxicity and lymphokine secretion mediated by cytotoxic T-lymphocyte (CTL) and NK cell. DAP10 (also known as HCST) is a transmembrane adapter protein which associates with KLRK1 to form an activation receptor KLRK1-HCST in lymphoid and myeloid cells; this receptor plays a major role in triggering cytotoxicity against target cells expressing cell surface ligands such as MHC class I chain-related MICA and MICB, and U(optionally L1)6-binding proteins (ULBPs); it KLRK1-HCST receptor plays a role in immune surveillance against tumors and is required for cytolysis of tumors cells; indeed, melanoma cells that do not express KLRK1 ligands escape from immune surveillance mediated by NK cells. CD16 is a receptor for the Fe region of IgG, which binds complexed or aggregated IgG and also monomeric IgG and thereby mediates antibody-dependent cellular cytotoxicity (ADCC) and other antibody-dependent responses, such as phagocytosis.

The present disclosure provides, inter alia, multispecific (e.g., bi-, tri-, quad-specific) or multifunctional molecules, that are engineered to contain one or more NK cell engagers that mediate binding to and/or activation of an NK cell. Accordingly, in some embodiments, the NK cell engager is selected from an antigen binding domain or ligand that binds to (e.g., activates): NKp30, NKp40, NKp44, NKp46, NKG2D, DNAM1, DAP10, CD16 (e.g., CD16a, CD16b, or both), CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS1, KIR2DS3, KIR2DS5, KIR2DS1, CD94, NKG2C, NKG2E, or CD160.

In some embodiments, the NK cell engager is an antigen binding domain that binds to NKp30 (e.g., NKp30 present, e.g., expressed or displayed, on the surface of an NK cell) and comprises any CDR amino acid sequence, framework region (FWR) amino acid sequence, or variable region amino acid sequence disclosed in Table 17, Table 20A or Table 20B, Table 22, Table 23A or Table 23B, Table 24, Table 25, Table 26, Table 21A or Table 21B, and Table 40. In some embodiments, the NK cell engager is an antigen binding domain that binds to NKp30 (e.g., NKp30 present, e.g., expressed or displayed, on the surface of an NK cell) and comprises any CDR amino acid sequence, framework region (FWR) amino acid sequence, or variable region amino acid sequence disclosed in U.S. Pat. Nos. 6,979,546, 9,447,185, PCT Application No. WO2015121383A1, PCT Application No. WO2016110468A1, PCT Application No. WO2004056392A1, or U.S. Application Publication No. US20070231322A1, the sequences of which are hereby incorporated by reference. In some embodiments, binding of the NK cell engager, e.g., antigen binding domain that binds to NKp30, to the NK cell activates the NK cell. An antigen binding domain that binds to NKp30 (e.g., NKp30 present, e.g., expressed or displayed, on the surface of an NK cell) may be said to target NKp30, the NK cell, or both.

In some embodiments, the antigen binding domain that binds to NKp30 comprises one or more CDRs (e.g., VHCDR1, VHCDR2, VHCDR3, VLCDR1, VLCDR2, and/or VLCDR3) disclosed in Table 20A or Table 20B, Table 21A or Table 21B, Table 22, or Table 40 or a sequence having at least 75%, 85%, 90%, 95%, or 99% identity thereto. In some embodiments, the antigen binding domain that binds to NKp30 comprises one or more framework regions (e.g., VHFWR1, VHFWR2, VHFWR3, VHFWR4, VLFWR1, VLFWR2, VLFWR3, and/or VLFWR4) disclosed in Table 20A or Table 20B, Table 21A or Table 21B, Table 22, or Table 40 or a sequence having at least 75%, 85%, 90%, 95%, or 99% identity thereto. In some embodiments, the antigen binding domain that binds to NKp30 comprises a VH and/or a VL disclosed in Table 25, or a sequence having at least 75%, 85%, 90%, 95%, or 99% identity thereto. In some embodiments, the antigen binding domain that binds to NKp30 comprises an amino acid sequence disclosed in Table 26 or Table 40, or a sequence having at least 75%, 85%, 90%, 95%, or 99% identity thereto.

In some embodiments, the antigen binding domain that binds to NKP30 comprises one or more CDRs (e.g., VHCDR1, VHCDR2, VHCDR3, VLCDR1, VLCDR2, and/or VLCDR3) disclosed in Table 23A, Table 23B, Table 24, or Table 40, or a sequence having at least 75%, 85%, 90%, 95%, or 99% identity thereto. In some embodiments, the antigen binding domain that binds to NKP30 comprises one or more framework regions (e.g., VHFWR1, VHFWR2, VHFWR3, VHFWR4, VLFWR1, VLFWR2, VLFWR3, and/or VLFWR4) disclosed in Table 23A, Table 23B, Table 24, or Table 40, or a sequence having at least 75%, 85%, 90%, 95%, or 99% identity thereto. In some embodiments, the antigen binding domain that binds to NKP30 comprises a VH and/or a VL disclosed in Table 25 or Table 40, or a sequence having at least 75%, 85%, 90%, 95%, or 99% identity thereto.

In some embodiments, the antigen binding domain that binds to NKp30 comprises a VH comprising a heavy chain complementarity determining region 1 (VHCDR1), a VHCDR2, and a VHCDR3, and a VL comprising a light chain complementarity determining region 1 (VLCDR1), a VLCDR2, and a VLCDR3.

In some embodiments, the VHCDR1, VHCDR2, and VHCDR3 comprise the amino acid sequences of SEQ ID NOs: 7313, 6001, and 7315, respectively (or a sequence having at least 75%, 85%, 90%, 95%, or 99% identity thereto). In some embodiments, the VHCDR1, VHCDR2, and VHCDR3 comprise the amino acid sequences of SEQ ID NOs: 7313, 6001, and 6002, respectively (or a sequence having at least 75%, 85%, 90%, 95%, or 99% identity thereto). In some embodiments, the VHCDR1, VHCDR2, and VHCDR3 comprise the amino acid sequences of SEQ ID NOs: 7313, 6008, and 6009, respectively (or a sequence having at least 75%, 85%, 90%, 95%, or 99% identity thereto). In some embodiments, the VHCDR1, VHCDR2, and VHCDR3 comprise the amino acid sequences of SEQ ID NOs: 7313, 7385, and 7315, respectively (or a sequence having at least 75%, 85%, 90%, 95%, or 99% identity thereto). In some embodiments, the VHCDR1, VHCDR2, and VHCDR3 comprise the amino acid sequences of SEQ ID NOs: 7313, 7318, and 6009, respectively (or a sequence having at least 75%, 85%, 90%, 95%, or 99% identity thereto). In some embodiments, the VHCDR1, VHCDR2, and VHCDR3 comprise the amino acid sequences of SEQ ID NOs: 19, 21, and 23, respectively (or a sequence having at least 75%, 85%, 90%, 95%, or 99% identity thereto). In some embodiments, the VHCDR1, VHCDR2, and VHCDR3 comprise the amino acid sequences of SEQ ID NOs: 33, 35, and 37, respectively (or a sequence having at least 75%, 85%, 90%, 95%, or 99% identity thereto). In some embodiments, the VHCDR1, VHCDR2, and VHCDR3 comprise the amino acid sequences of SEQ ID NOs: 47, 49, and 51, respectively (or a sequence having at least 75%, 85%, 90%, 95%, or 99% identity thereto). In some embodiments, the VHCDR1, VHCDR2, and VHCDR3 comprise the amino acid sequences of SEQ ID NOs: 61, 63, and 65, respectively (or a sequence having at least 75%, 85%, 90%, 95%, or 99% identity thereto). In some embodiments, the VHCDR1, VHCDR2, and VHCDR3 comprise the amino acid sequences of SEQ ID NOs: 7397, 313, and 79, respectively (or a sequence having at least 75%, 85%, 90%, 95%, or 99% identity thereto). In some embodiments, the VHCDR1, VHCDR2, and VHCDR3 comprise the amino acid sequences of SEQ ID NOs: 89, 91, and 93, respectively (or a sequence having at least 75%, 85%, 90%, 95%, or 99% identity thereto). In some embodiments, the VHCDR1, VHCDR2, and VHCDR3 comprise the amino acid sequences of SEQ ID NOs: 103, 313, and 107, respectively (or a sequence having at least 75%, 85%, 90%, 95%, or 99% identity thereto). In some embodiments, the VHCDR1, VHCDR2, and VHCDR3 comprise the amino acid sequences of SEQ ID NOs: 116, 118, and 120, respectively (or a sequence having at least 75%, 85%, 90%, 95%, or 99% identity thereto). In some embodiments, the VHCDR1, VHCDR2, and VHCDR3 comprise the amino acid sequences of SEQ ID NOs: 502, 503, and 504, respectively (or a sequence having at least 75%, 85%, 90%, 95%, or 99% identity thereto).

In some embodiments, the VLCDR1, VLCDR2, and VLCDR3 comprise the amino acid sequences of SEQ ID NOs: 26, 28, and 86, respectively (or a sequence having at least 75%, 85%, 90%, 95%, or 99% identity thereto). In some embodiments, the VLCDR1, VLCDR2, and VLCDR3 comprise the amino acid sequences of SEQ ID NOs: 6063, 6064, and 7293, respectively (or a sequence having at least 75%, 85%, 90%, 95%, or 99% identity thereto). In some embodiments, the VLCDR1, VLCDR2, and VLCDR3 comprise the amino acid sequences of SEQ ID NOs: 6070, 6071, and 6072, respectively (or a sequence having at least 75%, 85%, 90%, 95%, or 99% identity thereto). In some embodiments, the VLCDR1, VLCDR2, and VLCDR3 comprise the amino acid sequences of SEQ ID NOs: 6070, 6064, and 7321, respectively (or a sequence having at least 75%, 85%, 90%, 95%, or 99% identity thereto).

In some embodiments, the VHCDR1, VHCDR2, VHCDR3, VLCDR1, VLCDR2, and VLCDR3 comprise the amino acid sequences of SEQ ID NOs: 7313, 6001, 7315, 26, 28, and 86, respectively (or a sequence having at least 75%, 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7313, 6001, 6002, 6063, 6064, and 7293, respectively (or a sequence having at least 75%, 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7313, 6008, 6009, 6070, 6071, and 6072, respectively (or a sequence having at least 75%, 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7313, 7385, 7315, 6070, 6064, and 7321, respectively (or a sequence having at least 75%, 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 7313, 7318, 6009, 6070, 6064, and 7321, respectively (or a sequence having at least 75%, 85%, 90%, 95%, or 99% identity thereto). In some embodiments, the VHCDR1, VHCDR2, VHCDR3, VLCDR1, VLCDR2, and VLCDR3 comprise the amino acid sequences of SEQ ID NOs: 19, 21, 23, 26, 28, and 30, respectively (or a sequence having at least 75%, 85%, 90%, 95%, or 99% identity thereto) In some embodiments, the VHCDR1, VHCDR2, VHCDR3, VLCDR1, VLCDR2, and VLCDR3 comprise the amino acid sequences of SEQ ID NOs: 33, 35, 37, 40, 42, and 44, respectively (or a sequence having at least 75%, 85%, 90%, 95%, or 99% identity thereto) In some embodiments, the VHCDR1, VHCDR2, VHCDR3, VLCDR1, VLCDR2, and VLCDR3 comprise the amino acid sequences of SEQ ID NOs: 47, 49, 51, 54, 56, and 8678, respectively (or a sequence having at least 75%, 85%, 90%, 95%, or 99% identity thereto) In some embodiments, the VHCDR1, VHCDR2, VHCDR3, VLCDR1, VLCDR2, and VLCDR3 comprise the amino acid sequences of SEQ ID NOs: 61, 63, 65, 26, 28, and 72, respectively (or a sequence having at least 75%, 85%, 90%, 95%, or 99% identity thereto) In some embodiments, the VHCDR1, VHCDR2, VHCDR3, VLCDR1, VLCDR2, and VLCDR3 comprise the amino acid sequences of SEQ ID NOs: 7397, 313, 79, 26, 28, and 86, respectively (or a sequence having at least 75%, 85%, 90%, 95%, or 99% identity thereto) In some embodiments, the VHCDR1, VHCDR2, VHCDR3, VLCDR1, VLCDR2, and VLCDR3 comprise the amino acid sequences of SEQ ID NOs: 89, 91, 93, 26, 28, and 86, respectively (or a sequence having at least 75%, 85%, 90%, 95%, or 99% identity thereto) In some embodiments, the VHCDR1, VHCDR2, VHCDR3, VLCDR1, VLCDR2, and VLCDR3 comprise the amino acid sequences of SEQ ID NOs: 103, 313, 107, 26, 28, and 113, respectively (or a sequence having at least 75%, 85%, 90%, 95%, or 99% identity thereto) In some embodiments, the VHCDR1, VHCDR2, VHCDR3, VLCDR1, VLCDR2, and VLCDR3 comprise the amino acid sequences of SEQ ID NOs: 116, 118, 120, 26, 28, and 127, respectively (or a sequence having at least 75%, 85%, 90%, 95%, or 99% identity thereto).

In some embodiments, the VLCDR1, VLCDR2, and VLCDR3 comprise the amino acid sequences of SEQ ID NOs: 26, 28, and 30, respectively (or a sequence having at least 75%, 85%, 90%, 95%, or 99% identity thereto). In some embodiments, the VLCDR1, VLCDR2, and VLCDR3 comprise the amino acid sequences of SEQ ID NOs: 26, 42, and 7395, respectively (or a sequence having at least 75%, 85%, 90%, 95%, or 99% identity thereto). In some embodiments, the VLCDR1, VLCDR2, and VLCDR3 comprise the amino acid sequences of SEQ ID NOs: 26, 28, and 8678, respectively (or a sequence having at least 75%, 85%, 90%, 95%, or 99% identity thereto). In some embodiments, the VLCDR1, VLCDR2, and VLCDR3 comprise the amino acid sequences of SEQ ID NOs: 26, 28, and 72, respectively (or a sequence having at least 75%, 85%, 90%, 95%, or 99% identity thereto). In some embodiments, the VLCDR1, VLCDR2, and VLCDR3 comprise the amino acid sequences of SEQ ID NOs: 26, 28, and 86, respectively (or a sequence having at least 75%, 85%, 90%, 95%, or 99% identity thereto). In some embodiments, the VLCDR1, VLCDR2, and VLCDR3 comprise the amino acid sequences of SEQ ID NOs: C096, C098, and C100, respectively (or a sequence having at least 75%, 85%, 90%, 95%, or 99% identity thereto). In some embodiments, the VLCDR1, VLCDR2, and VLCDR3 comprise the amino acid sequences of SEQ ID NOs: 26, 28, and 113, respectively (or a sequence having at least 75%, 85%, 90%, 95%, or 99% identity thereto). In some embodiments, the VLCDR1, VLCDR2, and VLCDR3 comprise the amino acid sequences of SEQ ID NOs: 123, 28, and 127, respectively (or a sequence having at least 75%, 85%, 90%, 95%, or 99% identity thereto).

In some embodiments, the VH comprises an amino acid sequence selected from the group consisting of SEQ ID NOs: 505, 7298 or 7300-7304 (or a sequence having at least 75%, 85%, 90%, 95%, or 99% identity thereto) and/or the VL comprises an amino acid sequence selected from the group consisting of SEQ ID NOs: 13, 7299 or 7305-7308 (or a sequence having at least 75%, 85%, 90%, 95%, or 99% identity thereto). In some embodiments, the VH and VL comprise the amino acid sequences of SEQ ID NOs: 8030 and 7305, respectively (or a sequence having at least 75%, 85%, 90%, 95%, or 99% identity thereto). In some embodiments, the VH and VL comprise the amino acid sequences of SEQ ID NOs: 8030 and 13, respectively (or a sequence having at least 75%, 85%, 90%, 95%, or 99% identity thereto). In some embodiments, the VH and VL comprise the amino acid sequences of SEQ ID NOs: 505 and 13, respectively (or a sequence having at least 75%, 85%, 90%, 95%, or 99% identity thereto).

In some embodiments, the VH comprises an amino acid sequence selected from the group consisting of SEQ ID NOs: 6121 or 6123-6128 (or a sequence having at least 75%, 85%, 90%, 95%, or 99% identity thereto) and/or the VL comprises an amino acid sequence selected from the group consisting of SEQ ID NOs: 7294 or 6137-6141 (or a sequence having at least 75%, 85%, 90%, 95%, or 99% identity thereto). In some embodiments, the VH comprises an amino acid sequence selected from the group consisting of SEQ ID NOs: 6122 or 6129-6134 (or a sequence having at least 75%, 85%, 90%, 95%, or 99% identity thereto) and/or the VL comprises an amino acid sequence selected from the group consisting of SEQ ID NOs: 6136 or 6142-6147 (or a sequence having at least 75%, 85%, 90%, 95%, or 99% identity thereto). In some embodiments, the VH and VL comprise the amino acid sequences of SEQ ID NOs: 7295 and 7296, respectively (or a sequence having at least 75%, 85%, 90%, 95%, or 99% identity thereto). In some embodiments, the VH and VL comprise the amino acid sequences of SEQ ID NOs: 7297 and 7296, respectively (or a sequence having at least 75%, 85%, 90%, 95%, or 99% identity thereto). In some embodiments, the VH and VL comprise the amino acid sequences of SEQ ID NOs: 6122 and 6136, respectively (or a sequence having at least 75%, 85%, 90%, 95%, or 99% identity thereto).

In some embodiments, the antigen binding domain that binds to NKp30 comprises the amino acid sequence of SEQ ID NO: 7310 (or a sequence having at least 75%, 85%, 90%, 95%, or 99% identity thereto). In some embodiments, the antigen binding domain that binds to NKp30 comprises the amino acid sequence of SEQ ID NO: 7311 (or a sequence having at least 75%, 85%, 90%, 95%, or 99% identity thereto). In some embodiments, the antigen binding domain that binds to NKp30 comprises the amino acid sequence of SEQ ID NO: 6187, 6188, 6189 or 6190 (or a sequence having at least 75%, 85%, 90%, 95%, or 99% identity thereto). In some embodiments, the antigen binding domain that binds to NKp30 comprises the amino acid sequence of SEQ ID NO: 6187, 6188, 6189 or 6190 (or a sequence having at least 75%, 85%, 90%, 95%, or 99% identity thereto). In some embodiments, the antigen binding domain that binds to NKp30 comprises the amino acid sequence of SEQ ID NO: 518 (or a sequence having at least 75%, 85%, 90%, 95%, or 99% identity thereto).

In some embodiments, the antigen binding domain that targets NKp30 comprises a VH comprising a heavy chain complementarity determining region 1 (VHCDR1) amino acid sequence of SEQ ID NO: 6000 (or a sequence with no more than 1, 2, 3, or 4 mutations, e.g., substitutions, additions, or deletions), a VHCDR2 amino acid sequence of SEQ ID NO: 6001 (or a sequence with no more than 1, 2, 3, or 4 mutations, e.g., substitutions, additions, or deletions), and/or a VHCDR3 amino acid sequence of SEQ ID NO: 6002 (or a sequence with no more than 1, 2, 3, or 4 mutations, e.g., substitutions, additions, or deletions). In some embodiments, the NKp30 antigen binding domain comprises a VH comprising a VHCDR1 amino acid sequence of SEQ ID NO: 6000, a VHCDR2 amino acid sequence of SEQ ID NO: 6001, and/or a VHCDR3 amino acid sequence of SEQ ID NO: 6002.

In some embodiments, the antigen binding domain that targets NKp30 comprises a VH comprising a heavy chain complementarity determining region 1 (VHCDR1) amino acid sequence of SEQ ID NO: 502 (or a sequence with no more than 1, 2, 3, or 4 mutations, e.g., substitutions, additions, or deletions), a VHCDR2 amino acid sequence of SEQ ID NO: 503 (or a sequence with no more than 1, 2, 3, or 4 mutations, e.g., substitutions, additions, or deletions), and/or a VHCDR3 amino acid sequence of SEQ ID NO: 504 (or a sequence with no more than 1, 2, 3, or 4 mutations, e.g., substitutions, additions, or deletions). In some embodiments, the NKp30 antigen binding domain comprises a VH comprising a VHCDR1 amino acid sequence of SEQ ID NO: 502, a VHCDR2 amino acid sequence of SEQ ID NO: 503, and/or a VHCDR3 amino acid sequence of SEQ ID NO: 504.

In some embodiments, the antigen binding domain that targets NKp30 comprises a VL comprising a light chain complementarity determining region 1 (VLCDR1) amino acid sequence of SEQ ID NO: 6063 (or a sequence with no more than 1, 2, 3, or 4 mutations, e.g., substitutions, additions, or deletions), a VLCDR2 amino acid sequence of SEQ ID NO: 6064 (or a sequence with no more than 1, 2, 3, or 4 mutations, e.g., substitutions, additions, or deletions), and/or a VLCDR3 amino acid sequence of SEQ ID NO: 7293 (or a sequence with no more than 1, 2, 3, or 4 mutations, e.g., substitutions, additions, or deletions). In some embodiments, the antigen binding domain that targets NKp30 comprises a VL comprising a VLCDR1 amino acid sequence of SEQ ID NO: 6063, a VLCDR2 amino acid sequence of SEQ ID NO: 6064, and a VLCDR3 amino acid sequence of SEQ ID NO: 7293.

In some embodiments, the antigen binding domain that targets NKp30 comprises a VL comprising a light chain complementarity determining region 1 (VLCDR1) amino acid sequence of SEQ ID NO: 26 (or a sequence with no more than 1, 2, 3, or 4 mutations, e.g., substitutions, additions, or deletions), a VLCDR2 amino acid sequence of SEQ ID NO: 28 (or a sequence with no more than 1, 2, 3, or 4 mutations, e.g., substitutions, additions, or deletions), and/or a VLCDR3 amino acid sequence of SEQ ID NO: 86 (or a sequence with no more than 1, 2, 3, or 4 mutations, e.g., substitutions, additions, or deletions). In some embodiments, the antigen binding domain that targets NKp30 comprises a VL comprising a VLCDR1 amino acid sequence of SEQ ID NO: 26, a VLCDR2 amino acid sequence of SEQ ID NO: 28, and a VLCDR3 amino acid sequence of SEQ ID NO: 86.

In some embodiments, the antigen binding domain that targets NKp30 comprises a VH comprising a heavy chain complementarity determining region 1 (VHCDR1) amino acid sequence of SEQ ID NO: 6000 (or a sequence with no more than 1, 2, 3, or 4 mutations, e.g., substitutions, additions, or deletions), a VHCDR2 amino acid sequence of SEQ ID NO: 6001 (or a sequence with no more than 1, 2, 3, or 4 mutations, e.g., substitutions, additions, or deletions), and/or a VHCDR3 amino acid sequence of SEQ ID NO: 6002 (or a sequence with no more than 1, 2, 3, or 4 mutations, e.g., substitutions, additions, or deletions), and a VL comprising a light chain complementarity determining region 1 (VLCDR1) amino acid sequence of SEQ ID NO: 6063 (or a sequence with no more than 1, 2, 3, or 4 mutations, e.g., substitutions, additions, or deletions), a VLCDR2 amino acid sequence of SEQ ID NO: 6064 (or a sequence with no more than 1, 2, 3, or 4 mutations, e.g., substitutions, additions, or deletions), and/or a VLCDR3 amino acid sequence of SEQ ID NO: 7293 (or a sequence with no more than 1, 2, 3, or 4 mutations, e.g., substitutions, additions, or deletions). In some embodiments, the NKp30 antigen binding domain comprises a VH comprising a VHCDR1 amino acid sequence of SEQ ID NO: 6000, a VHCDR2 amino acid sequence of SEQ ID NO: 6001, and/or a VHCDR3 amino acid sequence of SEQ ID NO: 6002, and a VL comprising a VLCDR1 amino acid sequence of SEQ ID NO: 6063, a VLCDR2 amino acid sequence of SEQ ID NO: 6064, and a VLCDR3 amino acid sequence of SEQ ID NO: 7293.

In some embodiments, the antigen binding domain that targets NKp30 comprises a VH comprising a heavy chain complementarity determining region 1 (VHCDR1) amino acid sequence of SEQ ID NO: 6007 (or a sequence with no more than 1, 2, 3, or 4 mutations, e.g., substitutions, additions, or deletions), a VHCDR2 amino acid sequence of SEQ ID NO: 6008 (or a sequence with no more than 1, 2, 3, or 4 mutations, e.g., substitutions, additions, or deletions), and/or a VHCDR3 amino acid sequence of SEQ ID NO: 6009 (or a sequence with no more than 1, 2, 3, or 4 mutations, e.g., substitutions, additions, or deletions). In some embodiments, the NKp30 antigen binding domain comprises a VH comprising a VHCDR1 amino acid sequence of SEQ ID NO: 6007, a VHCDR2 amino acid sequence of SEQ ID NO: 6008, and/or a VHCDR3 amino acid sequence of SEQ ID NO: 6009.

In some embodiments, the antigen binding domain that targets NKp30 comprises a VL comprising a light chain complementarity determining region 1 (VLCDR1) amino acid sequence of SEQ ID NO: 6070 (or a sequence with no more than 1, 2, 3, or 4 mutations, e.g., substitutions, additions, or deletions), a VLCDR2 amino acid sequence of SEQ ID NO: 6071 (or a sequence with no more than 1, 2, 3, or 4 mutations, e.g., substitutions, additions, or deletions), and/or a VLCDR3 amino acid sequence of SEQ ID NO: 6072 (or a sequence with no more than 1, 2, 3, or 4 mutations, e.g., substitutions, additions, or deletions). In some embodiments, the antigen binding domain that targets NKp30 comprises a VL comprising a VLCDR1 amino acid sequence of SEQ ID NO: 6070, a VLCDR2 amino acid sequence of SEQ ID NO: 6071, and a VLCDR3 amino acid sequence of SEQ ID NO: 6072.

In some embodiments, the antigen binding domain that targets NKp30 comprises a VH comprising a heavy chain complementarity determining region 1 (VHCDR1) amino acid sequence of SEQ ID NO: 6007 (or a sequence with no more than 1, 2, 3, or 4 mutations, e.g., substitutions, additions, or deletions), a VHCDR2 amino acid sequence of SEQ ID NO: 6008 (or a sequence with no more than 1, 2, 3, or 4 mutations, e.g., substitutions, additions, or deletions), and/or a VHCDR3 amino acid sequence of SEQ ID NO: 6009 (or a sequence with no more than 1, 2, 3, or 4 mutations, e.g., substitutions, additions, or deletions), and a VL comprising a light chain complementarity determining region 1 (VLCDR1) amino acid sequence of SEQ ID NO: 6070 (or a sequence with no more than 1, 2, 3, or 4 mutations, e.g., substitutions, additions, or deletions), a VLCDR2 amino acid sequence of SEQ ID NO: 6071 (or a sequence with no more than 1, 2, 3, or 4 mutations, e.g., substitutions, additions, or deletions), and/or a VLCDR3 amino acid sequence of SEQ ID NO: 6072 (or a sequence with no more than 1, 2, 3, or 4 mutations, e.g., substitutions, additions, or deletions). In some embodiments, the NKp30 antigen binding domain comprises a VH comprising a VHCDR1 amino acid sequence of SEQ ID NO: 6007, a VHCDR2 amino acid sequence of SEQ ID NO: 6008, and/or a VHCDR3 amino acid sequence of SEQ ID NO: 6009, and a VL comprising a VLCDR1 amino acid sequence of SEQ ID NO: 6070, a VLCDR2 amino acid sequence of SEQ ID NO: 6071, and a VLCDR3 amino acid sequence of SEQ ID NO: 6072.

In some embodiments, the antigen binding domain that targets NKp30 comprises a VH comprising a heavy chain framework region 1 (VHFWR1) amino acid sequence of SEQ ID NO: 6003, a VHFWR2 amino acid sequence of SEQ ID NO: 6004, a VHFWR3 amino acid sequence of SEQ ID NO: 6005, and/or a VHFWR4 amino acid sequence of SEQ ID NO: 304.

In some embodiments, the antigen binding domain that targets NKp30 comprises a VL comprising a light chain framework region 1 (VLFWR1) amino acid sequence of SEQ ID NO: 6066, a VLFWR2 amino acid sequence of SEQ ID NO: 6067, a VLFWR3 amino acid sequence of SEQ ID NO: 7292, and/or a VLFWR4 amino acid sequence of SEQ ID NO: 6069.

In some embodiments, the antigen binding domain that targets NKp30 comprises a VH comprising a heavy chain framework region 1 (VHFWR1) amino acid sequence of SEQ ID NO: 6003, a VHFWR2 amino acid sequence of SEQ ID NO: 6004, a VHFWR3 amino acid sequence of SEQ ID NO: 6005, and/or a VHFWR4 amino acid sequence of SEQ ID NO: 304, and a VL comprising a light chain framework region 1 (VLFWR1) amino acid sequence of SEQ ID NO: 6066, a VLFWR2 amino acid sequence of SEQ ID NO: 6067, a VLFWR3 amino acid sequence of SEQ ID NO: 7292, and/or a VLFWR4 amino acid sequence of SEQ ID NO: 6069.

In some embodiments, the antigen binding domain that targets NKp30 comprises a VH comprising a VHFWR1 amino acid sequence of SEQ ID NO: 6003 (or a sequence with no more than 1, 2, 3, 4, 5, or 6 mutations, e.g., substitutions, additions, or deletions, therefrom), a VHFWR2 amino acid sequence of SEQ ID NO: 6004 (or a sequence with no more than 1, 2, 3, 4, 5, or 6 mutations, e.g., substitutions, additions, or deletions, therefrom), a VHFWR3 amino acid sequence of SEQ ID NO: 6005 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 304.

In some embodiments, the antigen binding domain that targets NKp30 comprises a VL comprising a VLFWR1 amino acid sequence of SEQ ID NO: 6066 (or a sequence with no more than 1, 2, or 3 mutations, e.g., substitutions, additions, or deletions), a VLFWR2 amino acid sequence of SEQ ID NO: 6067 (or a sequence with no more than 1 mutation, e.g., substitution, addition, or deletion), a VLFWR3 amino acid sequence of SEQ ID NO: 7292 (or a sequence with no more than 1 mutation, e.g., substitution, addition, or deletion), and/or a VLFWR4 amino acid sequence of SEQ ID NO: 6069.

In some embodiments, the antigen binding domain that targets NKp30 comprises a VH comprising a VHFWR1 amino acid sequence of SEQ ID NO: 6003 (or a sequence with no more than 1, 2, 3, 4, 5, or 6 mutations, e.g., substitutions, additions, or deletions, therefrom), a VHFWR2 amino acid sequence of SEQ ID NO: 6004 (or a sequence with no more than 1, 2, 3, 4, 5, or 6 mutations, e.g., substitutions, additions, or deletions, therefrom), a VHFWR3 amino acid sequence of SEQ ID NO: 6005 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 304, and a VL comprising a VLFWR1 amino acid sequence of SEQ ID NO: 6066 (or a sequence with no more than 1, 2, or 3 mutations, e.g., substitutions, additions, or deletions), a VLFWR2 amino acid sequence of SEQ ID NO: 6067 (or a sequence with no more than 1 mutation, e.g., substitution, addition, or deletion), a VLFWR3 amino acid sequence of SEQ ID NO: 7292 (or a sequence with no more than 1 mutation, e.g., substitution, addition, or deletion), and/or a VLFWR4 amino acid sequence of SEQ ID NO: 6069.

In some embodiments, the antigen binding domain that targets NKp30 comprises a VH comprising a heavy chain framework region 1 (VHFWR1) amino acid sequence of SEQ ID NO: 6010, a VHFWR2 amino acid sequence of SEQ ID NO: 6011, a VHFWR3 amino acid sequence of SEQ ID NO: 6012, and/or a VHFWR4 amino acid sequence of SEQ ID NO: 6013.

In some embodiments, the antigen binding domain that targets NKp30 comprises a VL comprising a light chain framework region 1 (VLFWR1) amino acid sequence of SEQ ID NO: 6073, a VLFWR2 amino acid sequence of SEQ ID NO: 6074, a VLFWR3 amino acid sequence of SEQ ID NO: 6075, and/or a VLFWR4 amino acid sequence of SEQ ID NO: 6076.

In some embodiments, the antigen binding domain that targets NKp30 comprises a VH comprising a heavy chain framework region 1 (VHFWR1) amino acid sequence of SEQ ID NO: 6010, a VHFWR2 amino acid sequence of SEQ ID NO: 6011, a VHFWR3 amino acid sequence of SEQ ID NO: 6012, and/or a VHFWR4 amino acid sequence of SEQ ID NO: 6013, and a VL comprising a light chain framework region 1 (VLFWR1) amino acid sequence of SEQ ID NO: 6073, a VLFWR2 amino acid sequence of SEQ ID NO: 6074, a VLFWR3 amino acid sequence of SEQ ID NO: 6075, and/or a VLFWR4 amino acid sequence of SEQ ID NO: 6076.

In some embodiments, the antigen binding domain that targets NKp30 comprises a VH comprising a VHFWR1 amino acid sequence of SEQ ID NO: 6010 (or a sequence with no more than 1, 2, 3, 4, 5, or 6 mutations, e.g., substitutions, additions, or deletions, therefrom), a VHFWR2 amino acid sequence of SEQ ID NO: 6011 (or a sequence with no more than 1, 2, 3, 4, 5, or 6 mutations, e.g., substitutions, additions, or deletions, therefrom), a VHFWR3 amino acid sequence of SEQ ID NO: 6012 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6013.

In some embodiments, the antigen binding domain that targets NKp30 comprises a VL comprising a VLFWR1 amino acid sequence of SEQ ID NO: 6073 (or a sequence with no more than 1, 2, or 3 mutations, e.g., substitutions, additions, or deletions), a VLFWR2 amino acid sequence of SEQ ID NO: 6074 (or a sequence with no more than 1 mutation, e.g., substitution, addition, or deletion), a VLFWR3 amino acid sequence of SEQ ID NO: 6075 (or a sequence with no more than 1 mutation, e.g., substitution, addition, or deletion), and/or a VLFWR4 amino acid sequence of SEQ ID NO: 6076.

In some embodiments, the antigen binding domain that targets NKp30 comprises a VH comprising a VHFWR1 amino acid sequence of SEQ ID NO: 6010 (or a sequence with no more than 1, 2, 3, 4, 5, or 6 mutations, e.g., substitutions, additions, or deletions, therefrom), a VHFWR2 amino acid sequence of SEQ ID NO: 6011 (or a sequence with no more than 1, 2, 3, 4, 5, or 6 mutations, e.g., substitutions, additions, or deletions, therefrom), a VHFWR3 amino acid sequence of SEQ ID NO: 6012 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6013, and a VL comprising a VLFWR1 amino acid sequence of SEQ ID NO: 6073 (or a sequence with no more than 1, 2, or 3 mutations, e.g., substitutions, additions, or deletions), a VLFWR2 amino acid sequence of SEQ ID NO: 6074 (or a sequence with no more than 1 mutation, e.g., substitution, addition, or deletion), a VLFWR3 amino acid sequence of SEQ ID NO: 6075 (or a sequence with no more than 1 mutation, e.g., substitution, addition, or deletion), and/or a VLFWR4 amino acid sequence of SEQ ID NO: 6076.

In some embodiments, the antigen binding domain that targets NKp30 comprises a VH comprising a heavy chain framework region 1 (VHFWR1) amino acid sequence of SEQ ID NO: 6014, a VHFWR2 amino acid sequence of SEQ ID NO: 6015, a VHFWR3 amino acid sequence of SEQ ID NO: 6016, and/or a VHFWR4 amino acid sequence of SEQ ID NO: 304.

In some embodiments, the antigen binding domain that targets NKp30 comprises a VH comprising a VHFWR1 amino acid sequence of SEQ ID NO: 6014 (or a sequence with no more than 1, 2, 3, 4, 5, or 6 mutations, e.g., substitutions, additions, or deletions, therefrom), a VHFWR2 amino acid sequence of SEQ ID NO: 6015 (or a sequence with no more than 1, 2, 3, 4, 5, or 6 mutations, e.g., substitutions, additions, or deletions, therefrom), a VHFWR3 amino acid sequence of SEQ ID NO: 6016 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 304.

In some embodiments, the antigen binding domain that targets NKp30 comprises a VL comprising a light chain framework region 1 (VLFWR1) amino acid sequence of SEQ ID NO: 6077, a VLFWR2 amino acid sequence of SEQ ID NO: 6078, a VLFWR3 amino acid sequence of SEQ ID NO: 6079, and/or a VLFWR4 amino acid sequence of SEQ ID NO: 6080.

In some embodiments, the antigen binding domain that targets NKp30 comprises a VL comprising a VLFWR1 amino acid sequence of SEQ ID NO: 6077 (or a sequence with no more than 1, 2, or 3 mutations, e.g., substitutions, additions, or deletions), a VLFWR2 amino acid sequence of SEQ ID NO: 6078 (or a sequence with no more than 1 mutation, e.g., substitution, addition, or deletion), a VLFWR3 amino acid sequence of SEQ ID NO: 6079 (or a sequence with no more than 1 mutation, e.g., substitution, addition, or deletion), and/or a VLFWR4 amino acid sequence of SEQ ID NO: 6080.

In some embodiments, the antigen binding domain that targets NKp30 comprises a VH comprising a heavy chain framework region 1 (VHFWR1) amino acid sequence of SEQ ID NO: 6018, a VHFWR2 amino acid sequence of SEQ ID NO: 6019, a VHFWR3 amino acid sequence of SEQ ID NO: 6020, and/or a VHFWR4 amino acid sequence of SEQ ID NO: 304.

In some embodiments, the antigen binding domain that targets NKp30 comprises a VH comprising a VHFWR1 amino acid sequence of SEQ ID NO: 6018 (or a sequence with no more than 1, 2, 3, 4, 5, or 6 mutations, e.g., substitutions, additions, or deletions, therefrom), a VHFWR2 amino acid sequence of SEQ ID NO: 6019 (or a sequence with no more than 1, 2, 3, 4, 5, or 6 mutations, e.g., substitutions, additions, or deletions, therefrom), a VHFWR3 amino acid sequence of SEQ ID NO: 6020 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 304.

In some embodiments, the antigen binding domain that targets NKp30 comprises a VL comprising a light chain framework region 1 (VLFWR1) amino acid sequence of SEQ ID NO: 6081, a VLFWR2 amino acid sequence of SEQ ID NO: 6082, a VLFWR3 amino acid sequence of SEQ ID NO: 6083, and/or a VLFWR4 amino acid sequence of SEQ ID NO: 6080.

In some embodiments, the antigen binding domain that targets NKp30 comprises a VL comprising a VLFWR1 amino acid sequence of SEQ ID NO: 6081 (or a sequence with no more than 1, 2, or 3 mutations, e.g., substitutions, additions, or deletions), a VLFWR2 amino acid sequence of SEQ ID NO: 6082 (or a sequence with no more than 1 mutation, e.g., substitution, addition, or deletion), a VLFWR3 amino acid sequence of SEQ ID NO: 6083 (or a sequence with no more than 1 mutation, e.g., substitution, addition, or deletion), and/or a VLFWR4 amino acid sequence of SEQ ID NO: 6080.

In some embodiments, the antigen binding domain that targets NKp30 comprises a VH comprising a heavy chain framework region 1 (VHFWR1) amino acid sequence of SEQ ID NO: 6022, a VHFWR2 amino acid sequence of SEQ ID NO: 6023, a VHFWR3 amino acid sequence of SEQ ID NO: 316, and/or a VHFWR4 amino acid sequence of SEQ ID NO: 304.

In some embodiments, the antigen binding domain that targets NKp30 comprises a VH comprising a VHFWR1 amino acid sequence of SEQ ID NO: 6022 (or a sequence with no more than 1, 2, 3, 4, 5, or 6 mutations, e.g., substitutions, additions, or deletions, therefrom), a VHFWR2 amino acid sequence of SEQ ID NO: 6023 (or a sequence with no more than 1, 2, 3, 4, 5, or 6 mutations, e.g., substitutions, additions, or deletions, therefrom), a VHFWR3 amino acid sequence of SEQ ID NO: 316 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 304.

In some embodiments, the antigen binding domain that targets NKp30 comprises a VL comprising a light chain framework region 1 (VLFWR1) amino acid sequence of SEQ ID NO: 6085, a VLFWR2 amino acid sequence of SEQ ID NO: 6086, a VLFWR3 amino acid sequence of SEQ ID NO: 6087, and/or a VLFWR4 amino acid sequence of SEQ ID NO: 6080.

In some embodiments, the antigen binding domain that targets NKp30 comprises a VL comprising a VLFWR1 amino acid sequence of SEQ ID NO: 6085 (or a sequence with no more than 1, 2, or 3 mutations, e.g., substitutions, additions, or deletions), a VLFWR2 amino acid sequence of SEQ ID NO: 6086 (or a sequence with no more than 1 mutation, e.g., substitution, addition, or deletion), a VLFWR3 amino acid sequence of SEQ ID NO: 6087 (or a sequence with no more than 1 mutation, e.g., substitution, addition, or deletion), and/or a VLFWR4 amino acid sequence of SEQ ID NO: 6080.

In some embodiments, the antigen binding domain that targets NKp30 comprises a VH comprising a heavy chain framework region 1 (VHFWR1) amino acid sequence of SEQ ID NO: 6026, a VHFWR2 amino acid sequence of SEQ ID NO: 6027, a VHFWR3 amino acid sequence of SEQ ID NO: 6028, and/or a VHFWR4 amino acid sequence of SEQ ID NO: 304.

In some embodiments, the antigen binding domain that targets NKp30 comprises a VH comprising a VHFWR1 amino acid sequence of SEQ ID NO: 6026 (or a sequence with no more than 1, 2, 3, 4, 5, or 6 mutations, e.g., substitutions, additions, or deletions, therefrom), a VHFWR2 amino acid sequence of SEQ ID NO: 6027 (or a sequence with no more than 1, 2, 3, 4, 5, or 6 mutations, e.g., substitutions, additions, or deletions, therefrom), a VHFWR3 amino acid sequence of SEQ ID NO: 6028 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 304.

In some embodiments, the antigen binding domain that targets NKp30 comprises a VL comprising a light chain framework region 1 (VLFWR1) amino acid sequence of SEQ ID NO: 6089, a VLFWR2 amino acid sequence of SEQ ID NO: 6090, a VLFWR3 amino acid sequence of SEQ ID NO: 6091, and/or a VLFWR4 amino acid sequence of SEQ ID NO: 6080.

In some embodiments, the antigen binding domain that targets NKp30 comprises a VL comprising a VLFWR1 amino acid sequence of SEQ ID NO: 6089 (or a sequence with no more than 1, 2, or 3 mutations, e.g., substitutions, additions, or deletions), a VLFWR2 amino acid sequence of SEQ ID NO: 6090 (or a sequence with no more than 1 mutation, e.g., substitution, addition, or deletion), a VLFWR3 amino acid sequence of SEQ ID NO: 6091 (or a sequence with no more than 1 mutation, e.g., substitution, addition, or deletion), and/or a VLFWR4 amino acid sequence of SEQ ID NO: 6080.

In some embodiments, the antigen binding domain that targets NKp30 comprises a VH comprising a heavy chain framework region 1 (VHFWR1) amino acid sequence of SEQ ID NO: 6030, a VHFWR2 amino acid sequence of SEQ ID NO: 6032, a VHFWR3 amino acid sequence of SEQ ID NO: 6033, and/or a VHFWR4 amino acid sequence of SEQ ID NO: 304.

In some embodiments, the antigen binding domain that targets NKp30 comprises a VH comprising a VHFWR1 amino acid sequence of SEQ ID NO: 6030 (or a sequence with no more than 1, 2, 3, 4, 5, or 6 mutations, e.g., substitutions, additions, or deletions, therefrom), a VHFWR2 amino acid sequence of SEQ ID NO: 6032 (or a sequence with no more than 1, 2, 3, 4, 5, or 6 mutations, e.g., substitutions, additions, or deletions, therefrom), a VHFWR3 amino acid sequence of SEQ ID NO: 6033 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 304.

In some embodiments, the antigen binding domain that targets NKp30 comprises a VL comprising a light chain framework region 1 (VLFWR1) amino acid sequence of SEQ ID NO: 6093, a VLFWR2 amino acid sequence of SEQ ID NO: 6094, a VLFWR3 amino acid sequence of SEQ ID NO: 6095, and/or a VLFWR4 amino acid sequence of SEQ ID NO: 6096.

In some embodiments, the antigen binding domain that targets NKp30 comprises a VL comprising a VLFWR1 amino acid sequence of SEQ ID NO: 6093 (or a sequence with no more than 1, 2, or 3 mutations, e.g., substitutions, additions, or deletions), a VLFWR2 amino acid sequence of SEQ ID NO: 6094 (or a sequence with no more than 1 mutation, e.g., substitution, addition, or deletion), a VLFWR3 amino acid sequence of SEQ ID NO: 6095 (or a sequence with no more than 1 mutation, e.g., substitution, addition, or deletion), and/or a VLFWR4 amino acid sequence of SEQ ID NO: 6096.

In some embodiments, the antigen binding domain that targets NKp30 comprises a VH comprising a heavy chain framework region 1 (VHFWR1) amino acid sequence of SEQ ID NO: 6035, a VHFWR2 amino acid sequence of SEQ ID NO: 6036, a VHFWR3 amino acid sequence of SEQ ID NO: 6037, and/or a VHFWR4 amino acid sequence of SEQ ID NO: 304.

In some embodiments, the antigen binding domain that targets NKp30 comprises a VH comprising a VHFWR1 amino acid sequence of SEQ ID NO: 6035 (or a sequence with no more than 1, 2, 3, 4, 5, or 6 mutations, e.g., substitutions, additions, or deletions, therefrom), a VHFWR2 amino acid sequence of SEQ ID NO: 6036 (or a sequence with no more than 1, 2, 3, 4, 5, or 6 mutations, e.g., substitutions, additions, or deletions, therefrom), a VHFWR3 amino acid sequence of SEQ ID NO: 6037 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 304.

In some embodiments, the antigen binding domain that targets NKp30 comprises a VH comprising a heavy chain framework region 1 (VHFWR1) amino acid sequence of SEQ ID NO: 6039, a VHFWR2 amino acid sequence of SEQ ID NO: 6040, a VHFWR3 amino acid sequence of SEQ ID NO: 6041, and/or a VHFWR4 amino acid sequence of SEQ ID NO: 6042.

In some embodiments, the antigen binding domain that targets NKp30 comprises a VH comprising a VHFWR1 amino acid sequence of SEQ ID NO: 6039 (or a sequence with no more than 1, 2, 3, 4, 5, or 6 mutations, e.g., substitutions, additions, or deletions, therefrom), a VHFWR2 amino acid sequence of SEQ ID NO: 6040 (or a sequence with no more than 1, 2, 3, 4, 5, or 6 mutations, e.g., substitutions, additions, or deletions, therefrom), a VHFWR3 amino acid sequence of SEQ ID NO: 6041 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6042.

In some embodiments, the antigen binding domain that targets NKp30 comprises a VL comprising a light chain framework region 1 (VLFWR1) amino acid sequence of SEQ ID NO: 6097, a VLFWR2 amino acid sequence of SEQ ID NO: 6098, a VLFWR3 amino acid sequence of SEQ ID NO: 6099, and/or a VLFWR4 amino acid sequence of SEQ ID NO: 6100.

In some embodiments, the antigen binding domain that targets NKp30 comprises a VL comprising a VLFWR1 amino acid sequence of SEQ ID NO: 6097 (or a sequence with no more than 1, 2, or 3 mutations, e.g., substitutions, additions, or deletions), a VLFWR2 amino acid sequence of SEQ ID NO: 6098 (or a sequence with no more than 1 mutation, e.g., substitution, addition, or deletion), a VLFWR3 amino acid sequence of SEQ ID NO: 6099 (or a sequence with no more than 1 mutation, e.g., substitution, addition, or deletion), and/or a VLFWR4 amino acid sequence of SEQ ID NO: 6100.

In some embodiments, the antigen binding domain that targets NKp30 comprises a VH comprising a heavy chain framework region 1 (VHFWR1) amino acid sequence of SEQ ID NO: 6043, a VHFWR2 amino acid sequence of SEQ ID NO: 6044, a VHFWR3 amino acid sequence of SEQ ID NO: 6045, and/or a VHFWR4 amino acid sequence of SEQ ID NO: 6046.

In some embodiments, the antigen binding domain that targets NKp30 comprises a VH comprising a VHFWR1 amino acid sequence of SEQ ID NO: 6043 (or a sequence with no more than 1, 2, 3, 4, 5, or 6 mutations, e.g., substitutions, additions, or deletions, therefrom), a VHFWR2 amino acid sequence of SEQ ID NO: 6044 (or a sequence with no more than 1, 2, 3, 4, 5, or 6 mutations, e.g., substitutions, additions, or deletions, therefrom), a VHFWR3 amino acid sequence of SEQ ID NO: 6045 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6046.

In some embodiments, the antigen binding domain that targets NKp30 comprises a VL comprising a light chain framework region 1 (VLFWR1) amino acid sequence of SEQ ID NO: 6101, a VLFWR2 amino acid sequence of SEQ ID NO: 6102, a VLFWR3 amino acid sequence of SEQ ID NO: 6103, and/or a VLFWR4 amino acid sequence of SEQ ID NO: 6104.

In some embodiments, the antigen binding domain that targets NKp30 comprises a VL comprising a VLFWR1 amino acid sequence of SEQ ID NO: 6101 (or a sequence with no more than 1, 2, or 3 mutations, e.g., substitutions, additions, or deletions), a VLFWR2 amino acid sequence of SEQ ID NO: 6102 (or a sequence with no more than 1 mutation, e.g., substitution, addition, or deletion), a VLFWR3 amino acid sequence of SEQ ID NO: 6103 (or a sequence with no more than 1 mutation, e.g., substitution, addition, or deletion), and/or a VLFWR4 amino acid sequence of SEQ ID NO: 6104.

In some embodiments, the antigen binding domain that targets NKp30 comprises a VH comprising a heavy chain framework region 1 (VHFWR1) amino acid sequence of SEQ ID NO: 6047, a VHFWR2 amino acid sequence of SEQ ID NO: 6048, a VHFWR3 amino acid sequence of SEQ ID NO: 316, and/or a VHFWR4 amino acid sequence of SEQ ID NO: 6050.

In some embodiments, the antigen binding domain that targets NKp30 comprises a VH comprising a VHFWR1 amino acid sequence of SEQ ID NO: 6047 (or a sequence with no more than 1, 2, 3, 4, 5, or 6 mutations, e.g., substitutions, additions, or deletions, therefrom), a VHFWR2 amino acid sequence of SEQ ID NO: 6048 (or a sequence with no more than 1, 2, 3, 4, 5, or 6 mutations, e.g., substitutions, additions, or deletions, therefrom), a VHFWR3 amino acid sequence of SEQ ID NO: 316 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6050.

In some embodiments, the antigen binding domain that targets NKp30 comprises a VL comprising a light chain framework region 1 (VLFWR1) amino acid sequence of SEQ ID NO: 6105, a VLFWR2 amino acid sequence of SEQ ID NO: 6106, a VLFWR3 amino acid sequence of SEQ ID NO: 6107, and/or a VLFWR4 amino acid sequence of SEQ ID NO: 6108.

In some embodiments, the antigen binding domain that targets NKp30 comprises a VL comprising a VLFWR1 amino acid sequence of SEQ ID NO: 6105 (or a sequence with no more than 1, 2, or 3 mutations, e.g., substitutions, additions, or deletions), a VLFWR2 amino acid sequence of SEQ ID NO: 6106 (or a sequence with no more than 1 mutation, e.g., substitution, addition, or deletion), a VLFWR3 amino acid sequence of SEQ ID NO: 6107 (or a sequence with no more than 1 mutation, e.g., substitution, addition, or deletion), and/or a VLFWR4 amino acid sequence of SEQ ID NO: 6108.

In some embodiments, the antigen binding domain that targets NKp30 comprises a VH comprising a heavy chain framework region 1 (VHFWR1) amino acid sequence of SEQ ID NO: 6051, a VHFWR2 amino acid sequence of SEQ ID NO: 6052, a VHFWR3 amino acid sequence of SEQ ID NO: 6053, and/or a VHFWR4 amino acid sequence of SEQ ID NO: 6054.

In some embodiments, the antigen binding domain that targets NKp30 comprises a VH comprising a VHFWR1 amino acid sequence of SEQ ID NO: 6051 (or a sequence with no more than 1, 2, 3, 4, 5, or 6 mutations, e.g., substitutions, additions, or deletions, therefrom), a VHFWR2 amino acid sequence of SEQ ID NO: 6052 (or a sequence with no more than 1, 2, 3, 4, 5, or 6 mutations, e.g., substitutions, additions, or deletions, therefrom), a VHFWR3 amino acid sequence of SEQ ID NO: 6053 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6054.

In some embodiments, the antigen binding domain that targets NKp30 comprises a VL comprising a light chain framework region 1 (VLFWR1) amino acid sequence of SEQ ID NO: 6109, a VLFWR2 amino acid sequence of SEQ ID NO: 6110, a VLFWR3 amino acid sequence of SEQ ID NO: 6111, and/or a VLFWR4 amino acid sequence of SEQ ID NO: 6112.

In some embodiments, the antigen binding domain that targets NKp30 comprises a VL comprising a VLFWR1 amino acid sequence of SEQ ID NO: 6109 (or a sequence with no more than 1, 2, or 3 mutations, e.g., substitutions, additions, or deletions), a VLFWR2 amino acid sequence of SEQ ID NO: 6110 (or a sequence with no more than 1 mutation, e.g., substitution, addition, or deletion), a VLFWR3 amino acid sequence of SEQ ID NO: 6111 (or a sequence with no more than 1 mutation, e.g., substitution, addition, or deletion), and/or a VLFWR4 amino acid sequence of SEQ ID NO: 6112.

In some embodiments, the antigen binding domain that targets NKp30 comprises a VH comprising a heavy chain framework region 1 (VHFWR1) amino acid sequence of SEQ ID NO: 6055, a VHFWR2 amino acid sequence of SEQ ID NO: 6056, a VHFWR3 amino acid sequence of SEQ ID NO: 6057, and/or a VHFWR4 amino acid sequence of SEQ ID NO: 6058.

In some embodiments, the antigen binding domain that targets NKp30 comprises a VH comprising a VHFWR1 amino acid sequence of SEQ ID NO: 6055 (or a sequence with no more than 1, 2, 3, 4, 5, or 6 mutations, e.g., substitutions, additions, or deletions, therefrom), a VHFWR2 amino acid sequence of SEQ ID NO: 6056 (or a sequence with no more than 1, 2, 3, 4, 5, or 6 mutations, e.g., substitutions, additions, or deletions, therefrom), a VHFWR3 amino acid sequence of SEQ ID NO: 6057 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6058.

In some embodiments, the antigen binding domain that targets NKp30 comprises a VL comprising a light chain framework region 1 (VLFWR1) amino acid sequence of SEQ ID NO: 6113, a VLFWR2 amino acid sequence of SEQ ID NO: 6114, a VLFWR3 amino acid sequence of SEQ ID NO: 6115, and/or a VLFWR4 amino acid sequence of SEQ ID NO: 6116.

In some embodiments, the antigen binding domain that targets NKp30 comprises a VL comprising a VLFWR1 amino acid sequence of SEQ ID NO: 6113 (or a sequence with no more than 1, 2, or 3 mutations, e.g., substitutions, additions, or deletions), a VLFWR2 amino acid sequence of SEQ ID NO: 6114 (or a sequence with no more than 1 mutation, e.g., substitution, addition, or deletion), a VLFWR3 amino acid sequence of SEQ ID NO: 6115 (or a sequence with no more than 1 mutation, e.g., substitution, addition, or deletion), and/or a VLFWR4 amino acid sequence of SEQ ID NO: 6116.

In some embodiments, the antigen binding domain that targets NKp30 comprises a VH comprising a heavy chain framework region 1 (VHFWR1) amino acid sequence of SEQ ID NO: 6059, a VHFWR2 amino acid sequence of SEQ ID NO: 6060, a VHFWR3 amino acid sequence of SEQ ID NO: 6061, and/or a VHFWR4 amino acid sequence of SEQ ID NO: 6062.

In some embodiments, the antigen binding domain that targets NKp30 comprises a VH comprising a VHFWR1 amino acid sequence of SEQ ID NO: 6059 (or a sequence with no more than 1, 2, 3, 4, 5, or 6 mutations, e.g., substitutions, additions, or deletions, therefrom), a VHFWR2 amino acid sequence of SEQ ID NO: 6060 (or a sequence with no more than 1, 2, 3, 4, 5, or 6 mutations, e.g., substitutions, additions, or deletions, therefrom), a VHFWR3 amino acid sequence of SEQ ID NO: 6061 (or a sequence with no more than 1, 2, 3, 4, 5, 6, 7, 8, 9, 10, or 11 mutations, e.g., substitutions, additions, or deletions), and/or a VHFWR4 amino acid sequence of SEQ ID NO: 6062.

In some embodiments, the antigen binding domain that targets NKp30 comprises a VL comprising a light chain framework region 1 (VLFWR1) amino acid sequence of SEQ ID NO: 6117, a VLFWR2 amino acid sequence of SEQ ID NO: 6118, a VLFWR3 amino acid sequence of SEQ ID NO: 6119, and/or a VLFWR4 amino acid sequence of SEQ ID NO: 6120.

In some embodiments, the antigen binding domain that targets NKp30 comprises a VL comprising a VLFWR1 amino acid sequence of SEQ ID NO: 6117 (or a sequence with no more than 1, 2, or 3 mutations, e.g., substitutions, additions, or deletions), a VLFWR2 amino acid sequence of SEQ ID NO: 6118 (or a sequence with no more than 1 mutation, e.g., substitution, addition, or deletion), a VLFWR3 amino acid sequence of SEQ ID NO: 6119 (or a sequence with no more than 1 mutation, e.g., substitution, addition, or deletion), and/or a VLFWR4 amino acid sequence of SEQ ID NO: 6120.

In some embodiments, the antigen binding domain that targets NKp30 comprises a VH comprising the amino acid sequence of SEQ ID NO: 6148 (or an amino acid sequence having at least about 77%, 80%, 85%, 90%, 95%, or 99% sequence identity to SEQ ID NO: 6148). In some embodiments, the antigen binding domain that targets NKp30 comprises a VH comprising the amino acid sequence of SEQ ID NO: 4688 (or an amino acid sequence having at least about 77%, 80%, 85%, 90%, 95%, or 99% sequence identity to SEQ ID NO: 4688). In some embodiments, the antigen binding domain that targets NKp30 comprises a VH comprising the amino acid sequence of SEQ ID NO: 6149 (or an amino acid sequence having at least about 77%, 80%, 85%, 90%, 95%, or 99% sequence identity to SEQ ID NO: 6149). In some embodiments, the antigen binding domain that targets NKp30 comprises a VH comprising the amino acid sequence of SEQ ID NO: 4689 (or an amino acid sequence having at least about 77%, 80%, 85%, 90%, 95%, or 99% sequence identity to SEQ ID NO: 4689). In some embodiments, the antigen binding domain that targets NKp30 comprises a VL comprising the amino acid sequence of SEQ ID NO: 6150 (or an amino acid sequence having at least about 93%, 95%, or 99% sequence identity to SEQ ID NO: 6150). In some embodiments, antigen binding domain that targets NKp30 comprises a VH comprising the amino acid sequence of SEQ ID NO: 6148. In some embodiments, antigen binding domain that targets NKp30 comprises a VH comprising the amino acid sequence of SEQ ID NO: 4866. In some embodiments, antigen binding domain that targets NKp30 comprises a VH comprising the amino acid sequence of SEQ ID NO: 6149. In some embodiments, antigen binding domain that targets NKp30 comprises a VH comprising the amino acid sequence of SEQ ID NO: 4689. In some embodiments, the antigen binding domain that targets NKp30 comprises a VL comprising the amino acid sequence of SEQ ID NO: 6150. In some embodiments, antigen binding domain that targets NKp30 comprises a VH comprising the amino acid sequence of SEQ ID NO: 505. In some embodiments, the antigen binding domain that targets NKp30 comprises a VL comprising the amino acid sequence of SEQ ID NO: 13.

In some embodiments, the antigen binding domain that targets NKp30 comprises a VH comprising the amino acid sequence of SEQ ID NO: 6148, and a VL comprising the amino acid sequence of SEQ ID NO: 6150. In some embodiments, the antigen binding domain that targets NKp30 comprises a VH comprising the amino acid sequence of SEQ ID NO: 4688, and a VL comprising the amino acid sequence of SEQ ID NO: 6150. In some embodiments, the antigen binding domain that targets NKp30 comprises a VH comprising the amino acid sequence of SEQ ID NO: 6149, and a VL comprising the amino acid sequence of SEQ ID NO: 6150. In some embodiments, the antigen binding domain that targets NKp30 comprises a VH comprising the amino acid sequence of SEQ ID NO: 4689, and a VL comprising the amino acid sequence of SEQ ID NO: 6150. In some embodiments, the antigen binding domain that targets NKp30 comprises a VH comprising the amino acid sequence of SEQ ID NO: 505, and a VL comprising the amino acid sequence of SEQ ID NO: 13.

In some embodiments, the antigen binding domain that targets NKp30 comprises a VH comprising the amino acid sequence of SEQ ID NO: 6151 (or an amino acid sequence having at least about 77%, 80%, 85%, 90%, 95%, or 99% sequence identity to SEQ ID NO: 6151). In some embodiments, the antigen binding domain that targets NKp30 comprises a VH comprising the amino acid sequence of SEQ ID NO: 4690 (or an amino acid sequence having at least about 77%, 80%, 85%, 90%, 95%, or 99% sequence identity to SEQ ID NO: 4690). In some embodiments, the antigen binding domain that targets NKp30 comprises a VH comprising the amino acid sequence of SEQ ID NO: 6152 (or an amino acid sequence having at least about 77%, 80%, 85%, 90%, 95%, or 99% sequence identity to SEQ ID NO: 6152). In some embodiments, the antigen binding domain that targets NKp30 comprises a VH comprising the amino acid sequence of SEQ ID NO: 6152 (or an amino acid sequence having at least about 77%, 80%, 85%, 90%, 95%, or 99% sequence identity to SEQ ID NO: 4691). In some embodiments, the antigen binding domain that targets NKp30 comprises a VL comprising the amino acid sequence of SEQ ID NO: 6153 (or an amino acid sequence having at least about 93%, 95%, or 99% sequence identity to SEQ ID NO: 6153). In some embodiments, antigen binding domain that targets NKp30 comprises a VH comprising the amino acid sequence of SEQ ID NO: 6151. In some embodiments, antigen binding domain that targets NKp30 comprises a VH comprising the amino acid sequence of SEQ ID NO: 4690. In some embodiments, antigen binding domain that targets NKp30 comprises a VH comprising the amino acid sequence of SEQ ID NO: 6152. In some embodiments, antigen binding domain that targets NKp30 comprises a VH comprising the amino acid sequence of SEQ ID NO: 4691. In some embodiments, the antigen binding domain that targets NKp30 comprises a VL comprising the amino acid sequence of SEQ ID NO: 6153.

In some embodiments, the antigen binding domain that targets NKp30 comprises a VH comprising the amino acid sequence of SEQ ID NO: 505 (or an amino acid sequence having at least about 75%, 80%, 85%, 90%, 95%, or 99% sequence identity to SEQ ID NO: 505). In some embodiments, the antigen binding domain that targets NKp30 comprises a VH comprising the amino acid sequence of SEQ ID NO: 505 (or an amino acid sequence having at least about 75%, 80%, 85%, 90%, 95%, or 99% sequence identity to SEQ ID NO: 13).

In some embodiments, the antigen binding domain that targets NKp30 comprises a VH comprising the amino acid sequence of SEQ ID NO: 6151, and a VL comprising the amino acid sequence of SEQ ID NO: 6153. In some embodiments, the antigen binding domain that targets NKp30 comprises a VH comprising the amino acid sequence of SEQ ID NO: 4690, and a VL comprising the amino acid sequence of SEQ ID NO: 6153. In some embodiments, the antigen binding domain that targets NKp30 comprises a VH comprising the amino acid sequence of SEQ ID NO: 6152, and a VL comprising the amino acid sequence of SEQ ID NO: 6153. In some embodiments, the antigen binding domain that targets NKp30 comprises a VH comprising the amino acid sequence of SEQ ID NO: 4691, and a VL comprising the amino acid sequence of SEQ ID NO: 6153.

In some embodiments, the antigen binding domain that targets NKp30 comprises an scFv. In some embodiments, the scFv comprises an amino acid sequence selected from SEQ ID NOs: 518, 6187-6190, or an amino acid sequence having at least about 93%, 95%, or 99% sequence identity thereto.

In some embodiments, the NK cell engager is an antigen binding domain that binds to NKp46 (e.g., NKp46 present, e.g., expressed or displayed, on the surface of an NK cell) and comprises any CDR amino acid sequence, framework region (FWR) amino acid sequence, or variable region amino acid sequence disclosed in Table 27. In some embodiments, binding of the NK cell engager, e.g., antigen binding domain that binds to NKp46, to the NK cell activates the NK cell. An antigen binding domain that binds to NKp46 (e.g., NKp46 present, e.g., expressed or displayed, on the surface of an NK cell) may be said to target NKp46, the NK cell, or both.

In some embodiments, the NK cell engager is an antigen binding domain that binds to NKG2D (e.g., NKG2D present, e.g., expressed or displayed, on the surface of an NK cell) and comprises any CDR amino acid sequence, framework region (FWR) amino acid sequence, or variable region amino acid sequence disclosed in Table 27. In some embodiments, binding of the NK cell engager, e.g., antigen binding domain that binds to NKG2D, to the NK cell activates the NK cell. An antigen binding domain that binds to NKG2D (e.g., NKG2D present, e.g., expressed or displayed, on the surface of an NK cell) may be said to target NKG2D, the NK cell, or both.

In some embodiments, the NK cell engager is an antigen binding domain that binds to CD16 (e.g., CD16 present, e.g., expressed or displayed, on the surface of an NK cell) and comprises any CDR amino acid sequence, framework region (FWR) amino acid sequence, or variable region amino acid sequence disclosed in Table 27. In some embodiments, binding of the NK cell engager, e.g., antigen binding domain that binds to CD16, to the NK cell activates the NK cell. An antigen binding domain that binds to CD16 (e.g., CD16 present, e.g., expressed or displayed, on the surface of an NK cell) may be said to target CD16, the NK cell, or both.

In one embodiment, the NK cell engager is a ligand of NKp30, e.g., is a B7-6, e.g., comprises the amino acid sequence of:

DLKVEMMAGGTQITPLNDNVTIFCNIFYSQPLNITSMGITWFWKSLTFDKEVKVFEFFGD HQEAFRPGAIVSPWRLKSGDASLRLPGIQLEEAGEYRCEVVVTPLKAQGTVQLEVVASPASRLLL DQVGMKENEDKYMCESSGFYPEAINITWEKQTQKFPHPIEISEDVITGPTIKNMDGTFNVTSCLK LNSSQEDPGTVYQCVVRHASLHTPLRSNFTLTAARHSLSETEKTDNFS (SEQ ID NO: 7233), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7233.

In other embodiments, the NK cell engager is a ligand of NKp44 or NKp46, which is a viral HA. Viral hemagglutinins (HA) are glyco proteins which are on the surface of viruses. HA proteins allow viruses to bind to the membrane of cells via sialic acid sugar moieties which contributes to the fusion of viral membranes with the cell membranes (see e.g., Eur J Immunol. 2001 September; 31(9):2680-9 “Recognition of viral hemagglutinins by NKp44 but not by NKp30”; and Nature. 2001 Feb. 22; 409(6823):1055-60 “Recognition of haemagglutinins on virus-infected cells by NKp46 activates lysis by human NK cells” the contents of each of which are incorporated by reference herein).

In other embodiments, the NK cell engager is a ligand of NKG2D chosen from MICA, MICB, or ULBP1, e.g., wherein:
(i) MICA comprises the amino acid sequence: EPHSLRYNLTVLSWDGSVQSGFLTEVHLDGQPFLRCDRQKCRAKPQGQWAEDVLGNKTWDRE TRDLTGNGKDLRMTLAHIKDQKEGLHSLQEIRVCEIHEDNSTRSSQHFYYDGELFLSQNLETKE WTMPQSSRAQTLAMNVRNFLKEDAMKTKTHYHAMHADCLQELRRYLKSGVVLRRTVPPMVN VTRSEASEGNITVTCRASGFYPWNITLSWRQDGVSLSHDTQQWGDVLPDGNGTYQTWVATRIC QGEEQRFTCYMEHSGNHSTHPVPSGKVLVLQSHW (SEQ ID NO: 7234), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7234;
(ii) MICB comprises the amino acid sequence: AEPHSLRYNLMVLSQDESVQSGFLAEGHLDGQPFLRYDRQKRRAKPQGQWAEDVLGAKTWDT ETEDLTENGQDLRRTLTHIKDQKGGLHSLQEIRVCEIHEDSSTRGSRHFYYDGELFLSQNLETQES TVPQSSRAQTLAMNVTNFWKEDAMKTKTHYRAMQADCLQKLQRYLKSGVAIRRTVPPMVNV TCSEVSEGNITVTCRASSFYPRNITLTWRQDGVSLSHNTQQWGDVLPDGNGTYQTWVATRIRQG EEQRFTCYMEHSGNHGTHPVPSGKVLVLQSQRTD (SEQ ID NO: 7235), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7235; or (iii) ULBP1 comprises the amino acid sequence:
GWVDTHCLCYDFIITPKSRPEPQWCEVQGLVDERPFLHYDCVNHKAKAFASLGKKVNVTKTWE EQTETLRDVVDFLKGQLLDIQVENLIPIEPLTLQARMSCEHEAHGHGRGSWQFLFNGQKFLLFDS NNRKWTALHPGAKKMTEKWEKNRDVTMFFQKISLGDCKMWLEEFLMYWEQMLDPTKPPSLA PG (SEQ ID NO: 7236), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7236.

In other embodiments, the NK cell engager is a ligand of DNAM1 chosen from NECTIN2 or NECL5, e.g., wherein:
(i) NECTIN2 comprises the amino acid sequence: QDVRVQVLPEVRGQLGGTVELPCHLLPPVPGLYISLVTWQRPDAPANHQNVAAFHPKMGPSFPS PKPGSERLSFVSAKQSTGQDTEAELQDATLALHGLTVEDEGNYTCEFATFPKGSVRGMTWLRVI AKPKNQAEAQKVTFSQDPTTVALCISKEGRPPARISWLSSLDWEAKETQVSGTLAGTVTVTSRFT LVPSGRADGVTVTCKVEHESFEEPALIPVTLSVRYPPEVSISGYDDNWYLGRTDATLSCDVRSNP EPTGYDWSTTSGTFPTSAVAQGSQLVIHAVDSLFNTTFVCTVTNAVGMGRAEQVIFVRETPNTA GAGATGG (SEQ ID NO: 7237), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7237; or
(ii) NECL5 comprises the amino acid sequence: WPPPGTGDVVVQAPTQVPGFLGDSVTLPCYLQVPNMEVTHVSQLTWARHGESGSMAVFHQTQ GPSYSESKRLEFVAARLGAELRNASLRMFGLRVEDEGNYTCLFVTFPQGSRSVDIWLRVLAKPQ NTAEVQKVQLTGEPVPMARCVSTGGRPPAQITWHSDLGGMPNTSQVPGFLSGTVTVTSLWILVP SSQVDGKNVTCKVEHESFEKPQLLTVNLTVYYPPEVSISGYDNNWYLGQNEATLTCDARSNPEP TGYNWSTTMGPLPPFAVAQGAQLLIRPVDKPINTTLICNVTNALGARQAELTVQVKEGPPSEHS GISRN (SEQ ID NO: 7238), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7238.

In yet other embodiments, the NK cell engager is a ligand of DAP10, which is an adapter for NKG2D (see e.g., Proc Natl Acad Sci USA. 2005 May 24; 102(21): 7641-7646; and Blood, 15 Sep. 2011 Volume 118, Number 11, the full contents of each of which is incorporated by reference herein).

In other embodiments, the NK cell engager is a ligand of CD16, which is a CD16a/b ligand, e.g., a CD16a/b ligand further comprising an antibody Fc region (see e.g., Front Immunol. 2013; 4: 76 discusses how antibodies use the Fe to trigger NK cells through CD16, the full contents of which are incorporated herein).

In other embodiments, the NK cell engager is a ligand of CRTAM, which is NECL2, e.g., wherein NECL2 comprises the amino acid sequence: QNLFTKDVTVIEGEVATISCQVNKSDDSVIQLLNPNRQTIYFRDFRPLKDSRFQLLNFSSSELKVS LTNVSISDEGRYFCQLYTDPPQESYTTITVLVPPRNLMIDIQKDTAVEGEEIEVNCTAMASKPATT IRWFKGNTELKGKSEVEEWSDMYTVTSQLMLKVHKEDDGVPVICQVEHPAVTGNLQTQRYLE VQYKPQVHIQMTYPLQGLTREGDALELTCEAIGKPQPVMVTWVRVDDEMPQHAVLSGPNLFIN NLNKTDNGTYRCEASNIVGKAHSDYMLYVYDPPTTIPPPTTTTTTTTTTTTTILTIITDSRAGEEGS IRAVDH (SEQ ID NO: 7239), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7239.

In other embodiments, the NK cell engager is a ligand of CD27, which is CD70, e.g., wherein CD70 comprises the amino acid sequence: QRFAQAQQQLPLESLGWDVAELQLNHTGPQQDPRLYWQGGPALGRSFLHGPELDKGQLRIHRD GIYMVHIQVTLAICSSTTASRHHPTTLAVGICSPASRSISLLRLSFHQGCTIASQRLTPLARGDTLC TNLTGTLLPSRNTDETFFGVQWVRP (SEQ ID NO: 7240), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7240.

In other embodiments, the NK cell engager is a ligand of PSGL1, which is L-selectin (CD62L), e.g., wherein L-selectin comprises the amino acid sequence: WTYHYSEKPMNWQRARRFCRDNYTDLVAIQNKAEIEYLEKTLPFSRSYYWIGIRKIGGIWTWV GTNKSLTEEAENWGDGEPNNKKNKEDCVEIYIKRNKDAGKWNDDACHKLKAALCYTASCQP WSCSGHGECVEIINNYTCNCDVGYYGPQCQFVIQCEPLEAPELGTMDCTHPLGNFSFSSQCAFSC SEGTNLTGIEETTCGPFGNWSSPEPTCQVIQCEPLSAPDLGIMNCSHPLASFSFTSACTFICSEGTEL IGKKKTICESSGIWSNPSPICQKLDKSFSMIKEGDYN (SEQ ID NO: 7241), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7241.

In other embodiments, the NK cell engager is a ligand of CD96, which is NECL5, e.g., wherein NECL5 comprises the amino acid sequence: WPPPGTGDVVVQAPTQVPGFLGDSVTLPCYLQVPNMEVTHVSQLTWARHGESGSMAVFHQTQ GPSYSESKRLEFVAARLGAELRNASLRMFGLRVEDEGNYTCLFVTFPQGSRSVDIWLRVLAKPQ NTAEVQKVQLTGEPVPMARCVSTGGRPPAQITWHSDLGGMPNTSQVPGFLSGTVTVTSLWILVP SSQVDGKNVTCKVEHESFEKPQLLTVNLTVYYPPEVSISGYDNNWYLGQNEATLTCDARSNPEP TGYNWSTTMGPLPPFAVAQGAQLLIRPVDKPINTTLICNVTNALGARQAELTVQVKEGPPSEHS GISRN (SEQ ID NO: 7238), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7238.

In other embodiments, the NK cell engager is a ligand of CD100 (SEMA4D), which is CD72, e.g., wherein CD72 comprises the amino acid sequence: RYLQVSQQLQQTNRVLEVTNSSLRQQLRLKITQLGQSAEDLQGSRRELAQSQEALQVEQRAHQ AAEGQLQACQADRQKTKETLQSEEQQRRALEQKLSNMENRLKPFFTCGSADTCCPSGWIMHQK SCFYISLTSKNWQESQKQCETLSSKLATFSEIYPQSHSYYFLNSLLPNGGSGNSYWTGLSSNKDW KLTDDTQRTRTYAQSSKCNKVHKTWSWWTLESESCRSSLPYICEMTAFRFPD (SEQ ID NO: 7242), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7242.

In other embodiments, the NK cell engager is a ligand of NKp80, which is CLEC2B (AICL), e.g., wherein CLEC2B (AICL) comprises the amino acid sequence: KLTRDSQSLCPYDWIGFQNKCYYFSKEEGDWNSSKYNCSTQHADLTIIDNIEEMNFLRRYKCSS DHWIGLKMAKNRTGQWVDGATFTKSFGMRGSEGCAYLSDDGAATARCYTERKWICRKRIH (SEQ ID NO: 7243), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7243.

In other embodiments, the NK cell engager is a ligand of CD244, which is CD48, e.g., wherein CD48 comprises the amino acid sequence: QGHLVHMTVVSGSNVTLNISESLPENYKQLTWFYTFDQKIVEWDSRKSKYFESKFKGRVRLDPQ SGALYISKVQKEDNSTYIMRVLKKTGNEQEWKIKLQVLDPVPKPVIKIEKIEDMDDNCYLKLSC VIPGESVNYTWYGDKRPFPKELQNSVLETTLMPHNYSRCYTCQVSNSVSSKNGTVCLSPPCTLA RS (SEQ ID NO: 7244), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7244.

In some embodiments, the NK cell engager is a viral hemagglutinin (HA), HA is a glycoprotein found on the surface of influenza viruses. It is responsible for binding the virus to cells with sialic acid on the membranes, such as cells in the upper respiratory tract or erythrocytes. HA has at least 18 different antigens. These subtypes are named H1 through H18. NCRs can recognize viral proteins. NKp46 has been shown to be able to interact with the HA of influenza and the HA-NA of Paramyxovirus, including Sendai virus and Newcastle disease virus. Besides NKp46, NKp44 can also functionally interact with HA of different influenza subtypes.

### T Cell Engagers

The present disclosure provides, inter alia, multispecific (e.g., bi-, tri-, quad-specific) or multifunctional molecules, that are engineered to contain one or more T cell engager that mediate binding to and/or activation of a T cell. Accordingly, in some embodiments, the T cell engager is selected from an antigen binding domain or ligand that binds to (e.g., and in some embodiments activates) one or more of CD3, TCRα, TCRβ, TCRγ, TCRζ, ICOS, CD28, CD27, HVEM, LIGHT, CD40, 4-4BB, OX40, DR3, GITR, CD30, TIM1, SLAM, CD2, or CD226. In other embodiments, the T cell engager is selected from an antigen binding domain or ligand that binds to and does not activate one or more of CD3, TCRa, TCRβ, TCRγ, TCRζ, ICOS, CD28, CD27, HVEM, LIGHT, CD40, 4-4BB, OX40, DR3, GITR, CD30, TIM1, SLAM, CD2, or CD226.

### TCR Beta V Antigen Binding Domains

In some embodiments, the TCRβ V5 subfamily is chosen from: TCRβ V5-5*01, TCRβ V5-6*01, TCRβ V5-4*01, TCRβ V5-8*01, TCRβ V5-1*01, or a variant thereof.

In some embodiments, the TCRβ V7 subfamily comprises TCRβ V7-7*01, TCRβ V7-6*01, TCRβ V7-8*02, TCRβ V7-4*01, TCRβ V7-2*02, TCRβ V7-2*03, TCRβ V7-2*01, TCRβ V7-3*01, TCRβ V7-9*03, or TCRβ V7-9*01, or a variant thereof.

In some embodiments, the TCRβ V11 subfamily comprises: TCRβ V11-1*01, TCRβ V11-2*01 or TCRβ V11-3*01, or a variant thereof. In some embodiments, the TCRβ V14 subfamily comprises TCRβ V14*01, or a variant thereof. In some embodiments, the TCRβ V16 subfamily comprises TCRβ V16*01, or a variant thereof. In some embodiments, the TCRβ V18 subfamily comprises TCRβ V18*01, or a variant thereof. In some embodiments, the TCRβ V9 subfamily comprises TCRβ V9*01 or TCRβ V9*02, or a variant thereof. In some embodiments, the TCRβ V13 subfamily comprises TCRβ V13*01, or a variant thereof. In some embodiments, the TCRβ V4 subfamily comprises TCRβ V4-2*01, TCRβ V4-3*01, or TCRβ V4-1*01, or a variant thereof. In some embodiments, the TCRβ V3 subfamily comprises TCRβ V3-1*01, or a variant thereof. In some embodiments, the TCRβ V2 subfamily comprises TCRβ V2*01, or a variant thereof. In some embodiments, the TCRβ V15 subfamily comprises TCRβ V15*01, or a variant thereof. In some embodiments, the TCRβ V30 subfamily comprises TCRβ V30*01, or TCRβ V30*02, or a variant thereof. In some embodiments, the TCRβ V19 subfamily comprises TCRβ V19*01, or TCRβ V19*02, or a variant thereof. In some embodiments, the TCRβ V27 subfamily comprises TCRβ V27*01, or a variant thereof. In some embodiments, the TCRβ V28 subfamily comprises TCRβ V28*01, or a variant thereof. In some embodiments, the TCRβ V24 subfamily comprises TCRβ V24-1*01, or a variant thereof. In some embodiments, the TCRβ V20 subfamily comprises TCRβ V20-1*01, or TCRβ V20-1*02, or a variant thereof. In some embodiments, the TCRβ V25 subfamily comprises TCRβ V25-1*01, or a variant thereof. In some embodiments, the TCRβ V29 subfamily comprises TCRβ V29-1*01, or a variant thereof.

Exemplary amino acid sequences for TCRβV subfamily members can be found on the ImMunoGeneTics Information System website: http://www.imgt.org/, or in a similar resource.

### Anti-TCRβV Antibodies

Current bispecific constructs designed to redirect T cells to promote tumor cell lysis for cancer immunotherapy typically utilize antibody fragments (Fab, scFv, VH, single domain antibody, etc.) that are derived from monoclonal antibodies (mAb) directed against the CD3e subunit of the T cell receptor (TCR). However, there are limitations to this approach which may prevent the full realization of the therapeutic potential for such bispecific constructs. Previous studies have shown that even low “activating” doses of anti-CD3e mAb can cause long-term T cell dysfunction and exert immunosuppressive effects. In addition, anti-CD3e mAbs have been associated with side effects that result from massive T cell activation. The large number of activated T cells secrete substantial amounts of cytokines, the most important of which is Interferon gamma (IFNγ). This excess amount of IFNγ in turn activates macrophages which then overproduce proinflammatory cytokines such as IL-1beta, IL-6, IL-10 and TNF-alpha, causing a “cytokine storm” known as the cytokine release syndrome (CRS) (Shimabukuro-Vornhagen et al., J Immunother Cancer. 2018 Jun. 15; 6(1):56, herein incorporated by reference in its entirety). Thus, the need exists for developing antibodies that are capable of binding and activating only a subset of effector T cells, e.g., to re-duce the CRS and/or neurotoxicity (NT).

Described herein are molecules targeting the TCRβV chain of TCR and methods thereof. Without wishing to be bound by theory, such molecules are capable of binding, activating, and/or expanding only a subset of T cells, avoiding or reducing CRS and/or NT and minimizing potential immunosuppressive effects of anti-CD3 mAbs.

Described herein is a class of antibodies, i.e., anti-TCRβV antibody molecules as described herein, which despite having low sequence similarity (e.g., low sequence identity among the different antibody molecules that recognize different TCRβV subfamilies), recognize a structurally conserved, yet sequence-wise variable, region, e.g., domain, on the TCRβV protein and have a similar function (e.g., activation of T cells and a similar cytokine profile as described herein). Thus, the anti-TCRβV antibody molecules as described herein share a structure-function relationship.

Without wishing to be bound by theory, in some embodiments, the anti-TCRβV antibody molecules as described herein bind to an outward facing epitope of a TCRβV protein when it is in a complex with a TCRalpha protein. In some embodiments, the anti-TCRβV antibody molecules as described herein recognize (e.g., bind to), a domain (e.g., an epitope) on the TCRβV protein that is: (1) structurally conserved among different TCRβV subfamilies; and (2) has minimal sequence identity among the different TCRβV subfamilies. TCRβV proteins from the different TCRβV subfamilies share minimal sequence similarity. However, TCRβV proteins which have minimal sequence similarity, share a similar 3D conformation and structure.

The alignment of TCRβV amino acid sequences underscores the diversity of TCR sequences. In particular, the TRBV sequences from different subfamilies are considerably different from each other.

In some embodiments, the anti-TCRβV antibody molecules as described herein do not recognize, e.g., bind to, an interface of a TCRβV:TCRalpha complex. In some embodiments, the anti-TCRβV antibody molecules as described herein do not recognize, e.g., bind to, a constant region of a TCRβV protein. An exemplary antibody that binds to a constant region of a TCRβV region is JOVI.1 as de-scribed in Viney et al., (Hybridoma. 1992 December; 11(6):701-13). In some embodiments, the anti-TCRβV antibody molecules as described herein do not recognize, e.g., bind to, one or more (e.g., all) of a complementarity determining region (e.g., CDR1, CDR2 and/or CDR3) of a TCRβV protein.

Provided herein are, inter alia, antibody molecules directed to the variable chain of the beta subunit of TCR (TCRβV) which bind and, e.g., activate a subset of T cells. The anti-TCRβV antibody molecules as described herein result in lesser or no production of cytokines associated with CRS, e.g., IL-6, IL-1beta, IL-10 and TNF alpha; and enhanced and/or delayed production of IL-2 and IFNγ. In some embodiments, the anti-TCRβV antibodies as described herein have a cytokine profile, e.g., as described herein, which differs from a cytokine profile of a T cell engager that binds to a receptor or molecule other than a TCRβV region (“a non-TCRβV-binding T cell engager”). In some embodiments, the non-TCRβV-binding T cell engager comprises an antibody that binds to a CD3 molecule (e.g., CD3 epsilon (CD3e) molecule); or a TCR alpha (TCRα) molecule. In some embodiments, the non-TCRβV-binding T cell engager is an OKT3 antibody or an SP34-2 antibody.

In some embodiments, the anti-TCRβV antibodies as described herein result in expansion of TCRβV+ T cells, e.g., a subset of memory effector T cells known as TEMRA. Without wishing to be bound by theory, it is believed that in some embodiments, TEMRA cells can promote tumor cell lysis but not CRS. Accordingly, provided herein are methods of making said anti-TCRβV antibody molecules and uses thereof. Also described herein are multispecific molecules, e.g., bispecific molecules comprising said anti-TCRβV antibody molecules. In some embodiments, compositions comprising anti-TCRβV antibody molecules of the present disclosure, can be used, e.g., to: (1) activate and redirect T cells to promote tumor cell lysis for cancer immuno-therapy; and/or (2) expand TCRβV+ T cells. In some embodiments, compositions comprising anti-TCRβV antibody molecules as described herein limit the harmful side-effects of CRS and/or NT, e.g., CRS and/or NT associated with anti-CD3e targeting.

In some embodiments, the anti-TCRβV antibody molecule binds to one or more of TRBV2, TRBV3-1, TRBV4-1, TRBV4-2, TRBV4-3, TRBV5-1, TRBV5-4, TRBV5-5, TRBV5-6, TRBV5-8, TRBV6-1, TRBV6-2, TRBV6-3, TRBV6-4, TRBV6-5, TRBV6-6, TRBV6-8, TRBV6-9, TRBV7-2, TRBV7-3, TRBV7-4, TRBV7-6, TRBV7-7, TRBV7-8, TRBV7-9, TRBV9, TRBV10-1, TRBV10-2, TRBV10-3, TRBV11-1, TRBV11-2, TRBV11-3, TRBV12-3, TRBV12-4, TRBV12-5, TRBV13, TRBV14, TRBV15, TRBV16, TRBV18, TRBV19, TRBV20-1, TRBV24-1, TRBV25-1, TRBV27, TRBV28, TRBV29-1 and TRBV30. In some embodiments, the anti-TCRβV antibody molecule binds to one or more of TRBV6-1, TRBV6-2, TRBV6-3, TRBV6-4, TRBV6-5, TRBV6-6, TRBV6-8 and TRBV6-9. In some embodiments, the anti-TCRβV antibody molecule is an anti-TRBV2, anti-TRBV3-1, anti-TRBV4-1, anti-TRBV4-2, anti-TRBV4-3, anti-TRBV5-1, anti-TRBV5-4, anti-TRBV5-5, anti-TRBV5-6, anti-TRBV5-8, anti-TRBV6-1, anti-TRBV6-2, anti-TRBV6-3, anti-TRBV6-4, anti-TRBV6-5, anti-TRBV6-6, anti-TRBV6-8, anti-TRBV6-9, anti-TRBV7-2, anti-TRBV7-3, anti-TRBV7-4, anti-TRBV7-6, anti-TRBV7-7, anti-TRBV7-8, anti-TRBV7-9, anti-TRBV9, anti-TRBV10-1, anti-TRBV10-2, anti-TRBV10-3, anti-TRBV11-1, anti-TRBV11-2, anti-TRBV11-3, anti-TRBV12-3, anti-TRBV12-4, anti-TRBV12-5, anti-TRBV13, anti-TRBV14, anti-TRBV15, anti-TRBV16, anti-TRBV18, anti-TRBV19, anti-TRBV20-1, anti-TRBV24-1, anti-TRBV25-1, anti-TRBV27, anti-TRBV28, anti-TRBV29-1, or anti-TRBV30. Exemplary anti-TCRβV antibody molecules and the corresponding TCRβV subfamilies recognized by said anti-TCRβV antibody molecules are disclosed in Table 42A.

In some embodiments, the anti-TCRβV antibody molecule binds specifically to TRBV2, TRBV3-1, TRBV4-1, TRBV4-2, TRBV4-3, TRBV5-1, TRBV5-4, TRBV5-5, TRBV5-6, TRBV5-8, TRBV6-1, TRBV6-2, TRBV6-3, TRBV6-4, TRBV6-5, TRBV6-6, TRBV6-8, TRBV6-9, TRBV7-2, TRBV7-3, TRBV7-4, TRBV7-6, TRBV7-7, TRBV7-8, TRBV7-9, TRBV9, TRBV10-1, TRBV10-2, TRBV10-3, TRBV11-1, TRBV11-2, TRBV11-3, TRBV12-3, TRBV12-4, TRBV12-5, TRBV13, TRBV14, TRBV15, TRBV16, TRBV18, TRBV19, TRBV20-1, TRBV24-1, TRBV25-1, TRBV27, TRBV28, TRBV29-1 or TRBV30. In some embodiments, the anti-TCRβV antibody molecule binds specifically to TRBV6-1. In some embodiments, the anti-TCRβV antibody molecule binds specifically to TRBV6-2. In some embodiments, the anti-TCRβV antibody molecule binds specifically to TRBV6-3. In some embodiments, the anti-TCRβV antibody molecule binds specifically to TRBV6-4. In some embodiments, the anti-TCRβV antibody molecule binds specifically to TRBV6-5. In some embodiments, the anti-TCRβV antibody molecule binds specifically to TRBV6-6. In some embodiments, the anti-TCRβV antibody molecule binds specifically to TRBV6-8. In some embodiments, the anti-TCRβV antibody molecule binds specifically to TRBV6-9.

In some embodiments, the anti-TCRβV antibody molecule does not bind to TCRβ V12, or binds to TCRβ V12 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the 16G8 murine antibody or a humanized version thereof as de-scribed in U.S. Pat. No. 5,861,155.

In some embodiments, the anti-TCRβV antibody molecule binds to TCRβ V12 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155.

In some embodiments, the anti-TCRβV antibody molecule binds to a TCRβV region other than TCRβ V12 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the 16G8 murine antibody or a humanized version thereof as de-scribed in U.S. Pat. No. 5,861,155.

In some embodiments, the anti-TCRβV antibody molecule does not comprise the CDRs of the Antibody B murine antibody.

In some embodiments, the anti-TCRβV antibody molecule does not bind to TCRβ V5-5*01 or TCRβ V5-1*01, or binds to TCRβ V5-5*01 or TCRβ V5-1*01 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the TM23 murine antibody or a humanized version thereof as described in U.S. Pat. No. 5,861,155.

In some embodiments, the anti-TCRβV antibody molecule binds to TCRβ V5-5*01 or TCRβ V5-1*01 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the TM23 murine antibody or a humanized version thereof as de-scribed in U.S. Pat. No. 5,861,155.

In some embodiments, the anti-TCRβV antibody molecule binds to a TCRβV region other than TCRβ V5-5*01 or TCRβ V5-1*01 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the TM23 murine antibody or a humanized version thereof as described in U.S. Pat. No. 5,861,155.

In some embodiments, the anti-TCRβV antibody molecule does not comprise the CDRs of the TM23 murine antibody.

In some embodiments, the light or the heavy chain variable framework (e.g., the region encompassing at least FR1, FR2, FR3, and optionally FR4) of the anti-TCRβV antibody molecule, e.g., anti-TCRβ V6 (e.g., anti-TCRβ V6-5*01) antibody molecule can be chosen from: (a) a light or heavy chain variable framework including at least 80%, 85%, 87% 90%, 92%, 93%, 95%, 97%, 98%, or 100% of the amino acid residues from a human light or heavy chain variable framework, e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, or a human consensus sequence; (c) a non-human framework (e.g., a rodent framework); or (d) a non-human framework that has been modified, e.g., to remove antigenic or cytotoxic determinants, e.g., deimmunized, or partially humanized. In some embodiments, the light or heavy chain variable framework region (particularly FR1, FR2 and/or FR3) includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

In some embodiments, the anti-TCRβV antibody molecule, e.g., anti-TCRβ V6 (e.g., anti-TCRβ V6-5*01) antibody molecule, comprises a heavy chain variable domain having at least one, two, three, four, five, six, seven, ten, fifteen, twenty or more changes, e.g., amino acid substitutions or deletions, from an amino acid sequence of any one of A-H.1 to A-H.85, e.g., A-H.1, A-H.2 or A-H.68, e.g., the amino acid sequence of the FR region in the entire variable region, e.g., in SEQ ID NO: 9.

Alternatively, or in combination with the heavy chain substitutions described herein, the anti-TCRβV antibody molecule, e.g., anti-TCRβ V6 (e.g., anti-TCRβ V6-5*01) antibody molecule, comprises a light chain variable domain having at least one, two, three, four, five, six, seven, ten, fifteen, twenty or more amino acid changes, e.g., amino acid substitutions or deletions, from an amino acid sequence of any one of A-H.1 to A-H.85, e.g., A-H.1, A-H.2 or A-H.68, e.g., the amino acid sequence of the FR region in the entire variable region, e.g., SEQ ID NO: 10 or SEQ ID NO: 11.

In some embodiments, the anti-TCRβV antibody molecule, e.g., anti-TCRβ V6 (e.g., anti-TCRβ V6-5*01) antibody molecule, includes one, two, three, or four heavy chain framework regions or a sequence substantially identical thereto.

In some embodiments, the anti-TCRβV antibody molecule, e.g., anti-TCRβ V6 (e.g., anti-TCRβ V6-5*01) antibody molecule, includes one, two, three, or four light chain framework regions or a sequence substantially identical thereto.

In some embodiments, the anti-TCRβV antibody molecule, e.g., anti-TCRβ V6 (e.g., anti-TCRβ V6-5*01) antibody molecule, comprises the light chain framework region 1 of A-H.1 or A-H.2.

In some embodiments, the anti-TCRβV antibody molecule, e.g., anti-TCRβ V6 (e.g., anti-TCRβ V6-5*01) antibody molecule, comprises the light chain framework region 2 of A-H.1 or A-H.2.

In some embodiments, the anti-TCRβV antibody molecule, e.g., anti-TCRβ V6 (e.g., anti-TCRβ V6-5*01) antibody molecule, comprises the light chain framework region 3 of A-H.1 or A-H.2.

In some embodiments, the anti-TCRβV antibody molecule, e.g., anti-TCRβ V6 (e.g., anti-TCRβ V6-5*01) antibody molecule, comprises the light chain framework region 4 of A-H.1 or A-H.2.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 1 (FR1), comprising a change, e.g., a substitution (e.g., a conservative substitution) at position 10 according to Kabat numbering. In some embodiments, the FR1 comprises a Phenylalanine at position 10, e.g., a Serine to Phenylalanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 2 (FR2), comprising a change, e.g., a substitution (e.g., a conservative substitution) at a position as described herein according to Kabat numbering. In some embodiments, FR2 comprises a Histidine at position 36, e.g., a substitution at position 36 according to Kabat numbering, e.g., a Tyrosine to Histidine substitution. In some embodiments, FR2 comprises an Alanine at position 46, e.g., a substitution at position 46 according to Kabat numbering, e.g., an Arginine to Alanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising a framework region, e.g., framework region 3 (FR3), comprising a change, e.g., a substitution (e.g., a conservative substitution) at a position as described herein according to Kabat numbering. In some embodiments, FR3 comprises a Phenylalanine at position 87, e.g., a substitution at position 87 according to Kabat numbering, e.g., a Tyrosine to Phenylalanine substitution. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 1 (FR1) comprising a Phenylalanine at position 10, e.g., a substitution at position 10 according to Kabat numbering, e.g., a Serine to Phenylalanine substitution; (b) a framework region 2 (FR2) comprising a Histidine at position 36, e.g., a substitution at position 36 according to Kabat numbering, e.g., a Tyrosine to Histidine substitution, and a Alanine at position 46, e.g., a substitution at position 46 according to Kabat numbering, e.g., a Arginine to Alanine substitution; and (c) a framework region 3 (FR3) comprising a Phenylalanine at position 87, e.g., a substitution at position 87 according to Kabat numbering, e.g., a Tyrosine to Phenylalanine substitution, e.g., as shown in the amino acid sequence of SEQ ID NO: 10. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 2 (FR2) comprising a Histidine at position 36, e.g., a substitution at position 36 according to Kabat numbering, e.g., a Tyrosine to Histidine substitution, and a Alanine at position 46, e.g., a substitution at position 46 according to Kabat numbering, e.g., a Arginine to Alanine substitution; and (b) a framework region 3 (FR3) comprising a Phenylalanine at position 87, e.g., a substitution at position 87 according to Kabat numbering, e.g., a Tyrosine to Phenylalanine substitution, e.g., as shown in the amino acid sequence of SEQ ID NO: 11. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises a light chain variable domain comprising: (a) a framework region 1 (FR1) comprising a change, e.g., a substitution (e.g., a conservative substitution) at one or more (e.g., all) positions as described herein according to Kabat numbering,; (b) a framework region 2 (FR2) comprising a change, e.g., a substitution (e.g., a conservative substitution) at one or more (e.g., all) position as described herein according to Kabat numbering and (c) a framework region 3 (FR3) comprising a change, e.g., a substitution (e.g., a conservative substitution) at one or more (e.g., all) position as described herein according to Kabat numbering. In some embodiments, the substitution is relative to a human germline light chain framework region sequence.

In some embodiments, the anti-TCRβV antibody molecule, e.g., anti-TCRβ V6 (e.g., anti-TCRβ V6-5*01) antibody molecule, comprises the heavy chain framework region 1 of A-H.1 or A-H.2. In some embodiments, the anti-TCRβV antibody molecule, e.g., anti-TCRβ V6 (e.g., anti-TCRβ V6-5*01) antibody molecule, comprises the heavy chain framework region 2 of A-H.1 or A-H.2. In some embodiments, the anti-TCRβV antibody molecule, e.g., anti-TCRβ V6 (e.g., anti-TCRβ V6-5*01) antibody molecule, comprises the heavy chain framework region 3 of A-H.1 or A-H.2. In some embodiments, the anti-TCRβV antibody molecule, e.g., anti-TCRβ V6 (e.g., anti-TCRβ V6-5*01) antibody molecule, comprises the heavy chain framework region 4 of A-H.1 or A-H.2.

In some embodiments, the anti-TCRβV antibody molecule, e.g., anti-TCRβ V6 (e.g., anti-TCRβ V6-5*01) antibody molecule, comprises a heavy chain variable domain comprising a framework region, e.g., framework region 3 (FR3), comprising a change, e.g., a substitution (e.g., a conservative substitution) at a position as described herein according to Kabat numbering. In some embodiments, FR3 comprises a Threonine at position 73, e.g., a substitution at position 73 according to Kabat numbering, e.g., a Glutamic Acid to Threonine substitution. In some embodiments, FR3 comprises a Glycine at position 94, e.g., a substitution at position 94 according to Kabat numbering, e.g., an Arginine to Glycine substitution. In some embodiments, the substitution is relative to a human germline heavy chain framework region sequence.

In some embodiments, the anti-TCRβV antibody molecule, e.g., anti-TCRβ V6 (e.g., anti-TCRβ V6-5*01) antibody molecule, comprises a heavy chain variable domain comprising a framework region 3 (FR3) comprising a Threonine at position 73, e.g., a substitution at position 73 according to Kabat numbering, e.g., a Glutamic Acid to Threonine substitution, and a Glycine at position 94, e.g., a substitution at position 94 according to Kabat numbering, e.g., a Arginine to Glycine substitution, e.g., as shown in the amino acid sequence of SEQ ID NO: 10.

In some embodiments, the anti-TCRβV antibody molecule, e.g., anti-TCRβ V6 (e.g., anti-TCRβ V6-5*01) antibody molecule, comprises the heavy chain framework regions 1-4 of A-H.1 or A-H.2, e.g., SEQ ID NO: 9. In some embodiments, the anti-TCRβV antibody molecule, e.g., anti-TCRβ V6 (e.g., anti-TCRβ V6-5*01) antibody molecule, comprises the light chain framework regions 1-4 of A-H.1, e.g., SEQ ID NO: 10. In some embodiments, the anti-TCRβV antibody molecule, e.g., anti-TCRβ V6 (e.g., anti-TCRβ V6-5*01) antibody molecule, comprises the light chain framework regions 1-4 of A-H.2, e.g., SEQ ID NO: 11. In some embodiments, the anti-TCRβV antibody molecule, e.g., anti-TCRβ V6 (e.g., anti-TCRβ V6-5*01) antibody molecule, comprises the heavy chain framework regions 1-4 of A-H.1, e.g., SEQ ID NO: 9; and the light chain framework regions 1-4 of A-H.1, e.g., SEQ ID NO: 10. In some embodiments, the anti-TCRβV antibody molecule, e.g., anti-TCRβ V6 (e.g., anti-TCRβ V6-5*01) antibody molecule, comprises the heavy chain framework regions 1-4 of A-H.2, e.g., SEQ ID NO: 9; and the light chain framework regions 1-4 of A-H.2, e.g., SEQ ID NO: 11.

In some embodiments, the heavy or light chain variable domain, or both, of the anti-TCRβV antibody molecule, e.g., anti-TCRβ V6 (e.g., anti-TCRβ V6-5*01) antibody molecule, includes an amino acid sequence, which is substantially identical to an amino acid as described herein, e.g., at least 80%, 85%, 90%, 92%, 95%, 97%, 98%, 99% or higher identical to a variable region of an antibody described herein, e.g., an antibody chosen from any one of A-H.1 to A-H.85, e.g., A-H.1, A-H.2 or A-H.68, or as described in Table 30, or encoded by the nucleotide sequence in Table 30; or which differs at least 1 or 5 residues, but less than 40, 30, 20, or 10 residues, from a variable region of an antibody described herein.

In some embodiments, the anti-TCRβV antibody molecule, e.g., anti-TCRβ V6 (e.g., anti-TCRβ V6-5*01) antibody molecule, comprises at least one, two, three, or four antigen-binding regions, e.g., variable regions, having an amino acid sequence as set forth in Table 1, or a sequence substantially identical thereto (e.g., a sequence at least about 85%, 90%, 95%, 99% or more identical thereto, or which differs by no more than 1, 2, 5, 10, or 15 amino acid residues from the sequences shown in Table 1. In another embodiment, the anti-TCRβV antibody molecule, e.g., anti-TCRβ V6 (e.g., anti-TCRβ V6-5*01) antibody molecule includes a VH and/or VL domain encoded by a nucleic acid having a nucleotide sequence as set forth in Table 1, or a sequence substantially identical thereto (e.g., a sequence at least about 85%, 90%, 95%, 99% or more identical thereto, or which differs by no more than 3, 6, 15, 30, or 45 nucleotides from the sequences shown in Table 1.

In some embodiments, the anti-TCRβV antibody molecule, e.g., anti-TCRβ V6 (e.g., anti-TCRβ V6-5*01) antibody molecule, comprises: a VH domain comprising the amino acid sequence of SEQ ID NO: 9, an amino acid sequence at least about 85%, 90%, 95%, 99% or more identical to the amino acid sequence of SEQ ID NO: 9, or an amino acid sequence which differs by no more than 1, 2, 5, 10, or 15 amino acid residues from the amino acid sequence of SEQ ID NO: 9; and/or a VL domain comprising the amino acid sequence of SEQ ID NO: 10, an amino acid sequence at least about 85%, 90%, 95%, 99% or more identical to the amino acid sequence of SEQ ID NO: 10, or an amino acid sequence which differs by no more than 1, 2, 5, 10, or 15 amino acid residues from the amino acid sequence of SEQ ID NO: 10.

In some embodiments, the anti-TCRβV antibody molecule, e.g., anti-TCRβ V6 (e.g., anti-TCRβ V6-5*01) antibody molecule, comprises: a VH domain comprising the amino acid sequence of SEQ ID NO: 9, an amino acid sequence at least about 85%, 90%, 95%, 99% or more identical to the amino acid sequence of SEQ ID NO: 9, or an amino acid sequence which differs by no more than 1, 2, 5, 10, or 15 amino acid residues from the amino acid sequence of SEQ ID NO: 9; and/or a VL domain comprising the amino acid sequence of SEQ ID NO: 11, an amino acid sequence at least about 85%, 90%, 95%, 99% or more identical to the amino acid sequence of SEQ ID NO: 11, or an amino acid sequence which differs by no more than 1, 2, 5, 10, or 15 amino acid residues from the amino acid sequence of SEQ ID NO: 11.

In some embodiments, the anti-TCRβV antibody molecule, e.g., anti-TCRβ V6 (e.g., anti-TCRβ V6-5*01) antibody molecule is a full antibody or fragment thereof (e.g., a Fab, F(ab′)₂, Fv, single domain antibody, or a single chain Fv fragment (scFv)). In embodiments, the anti-TCRβV antibody molecule, e.g., anti-TCRβ V6 (e.g., anti-TCRβ V6-5*01) antibody molecule is a monoclonal antibody or an antibody with single specificity. In some embodiments, the anti-TCRβV antibody molecule, e.g., anti-TCRβ V6 (e.g., anti-TCRβ V6-5*01) antibody molecule, can also be a humanized, chimeric, camelid, shark, or an in vitro-generated antibody molecule. In some embodiments, the anti-TCRβV antibody molecule, e.g., anti-TCRβ V6 (e.g., anti-TCRβ V6-5*01) antibody molecule, is a humanized antibody molecule. The heavy and light chains of the anti-TCRβV antibody molecule, e.g., anti-TCRβ V6 (e.g., anti-TCRβ V6-5*01) antibody molecule, can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRβV antibody molecule, e.g., anti-TCRβ V6 (e.g., anti-TCRβ V6-5*01) antibody molecule, is in the form of a multispecific molecule, e.g., a bispecific molecule, e.g., as described herein.

In some embodiments, the anti-TCRβV antibody molecule, e.g., anti-TCRβ V6 (e.g., anti-TCRβ V6-5*01) antibody molecule, has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1. In some embodiments, the Fc region comprises a Fc region variant, e.g., as described herein.

In some embodiments, the anti-TCRβV antibody molecule, e.g., anti-TCRβ V6 (e.g., anti-TCRβ V6-5*01) antibody molecule, has a light chain constant region chosen from, e.g., the light chain constant regions of kappa or lambda, preferably kappa (e.g., human kappa). In some embodiments, the constant region is altered, e.g., mutated, to modify the properties of the anti-TCRβV antibody molecule, e.g., anti-TCRβ V6 (e.g., anti-TCRβ V6-5*01) antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) according to EU Numbering to alter Fc receptor binding (e.g., the mutated positions correspond to positions positions 135 (M to Y), 137 (S to T), 139 (T to E), 316 (H to K) and 317 (N to F) of SEQ ID NOs: 41 or 3647), e.g., relative to human IgG1.

Antibody A-H.1 comprises a heavy chain comprising the amino acid sequence of SEQ ID NO: 3278 and a light chain comprising the amino acid sequence of SEQ ID NO: 72. Antibody A-H.2 comprises a heavy chain comprising the amino acid sequence of SEQ ID NO: 3278 and a light chain comprising the amino acid sequence of SEQ ID NO: 3279. Antibody A-H.68 comprises the amino acid sequence of SEQ ID NO: 1337, or a sequence having at least 85%, 90%, 95%, 96%, 97%, 98%, 99%, 99.5%, 99.9%, or 100% sequence identity thereto. Antibody A-H.69 comprises the amino acid sequence of SEQ ID NO: 1500, or a sequence having at least 85%, 90%, 95%, 96%, 97%, 98%, 99%, 99.5%, 99.9%, or 100% sequence identity thereto.

Additional exemplary humanized anti-TCRB V6 antibodies are provided in Table 1. In some embodiments, the anti-TCRβ V6 is antibody A, e.g., humanized antibody A (antibody A-H), as provided in Table 1. In some embodiments, the anti-TCRβV antibody comprises one or more (e.g., all three) of a LC CDR1, LC CDR2, and LC CDR3 provided in Table 1; and/or one or more (e.g., all three) of a HC CDR1, HC CDR2, and HC CDR3 provided in Table 1, or a sequence with at least 85%, 90%, 95%, 96%, 97%, 98%, 99%, 99.5%, 99.9%, or 100% sequence identity thereto. In some embodiments, antibody A comprises a variable heavy chain (VH) and/or a variable light chain (VL) provided in Table 1, or a sequence with at least 85%, 90%, 95%, 96%, 97%, 98%, 99%, 99.5%, 99.9%, or 100% sequence identity thereto.

In some embodiments, the anti-TCRβV antibody molecule, e.g., anti-TCRβ V6 (e.g., anti-TCRβ V6-5*01) antibody molecule comprises a VH of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VL of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VH of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto; and a VL of A-H.1, A-H.2, A-H.3, A-H.4, A-H.5, A-H.6, A-H.7, A-H.8, A-H.9, A-H.10, A-H.11, A-H.12, A-H.13, A-H.14, A-H.15, A-H.16, A-H.17, A-H.18, A-H.19, A-H.20, A-H.21, A-H.22, A-H.23, A-H.24, A-H.25, A-H.26, A-H.27, A-H.28, A-H.29, A-H.30, A-H.31, A-H.32, A-H.33, A-H.34, A-H.35, A-H.36, A-H.37, A-H.38, A-H.39, A-H.40, A-H.1, A-H.42, A-H.43, A-H.44, A-H.45, A-H.46, A-H.47, A-H.48, A-H.49, A-H.50, A-H.51, A-H.52, A-H.53, A-H.54, A-H.55, A-H.56, A-H.57, A-H.58, A-H.59, A-H.60, A-H.61, A-H.62, A-H.63, A-H.64, A-H.65, A-H.66, A-H.67, A-H.68, A-H.69, A-H.70, A-H.71, A-H.72, A-H.73, A-H.74, A-H.75, A-H.76, A-H.77, A-H.78, A-H.79, A-H.80, A-H.81, A-H.82, A-H.83, A-H.84, or A-H.85, or a sequence with at least 80%, 85%, 90%, 95%, 96%, 97%, 98%, 99% or more identity thereto.

Exemplary anti-TCRβV antibody molecules and the corresponding TCRβV subfamilies recognized by said anti-TCRβV antibody molecules are disclosed in Table 42A.

The various TCRβV subfamilies and/or subfamily members can be expressed at different levels in individuals, e.g., healthy individuals, as disclosed in Kitaura K. et al (2016), BMC Immunology vol 17: 38, the entire contents of which are hereby incorporated by reference. For example, TCRβ V6-5 is represented in approximately 3-6% healthy donors.

The representation of various TCRβV subfamilies and/or subfamily members can also be different in cancer cells. For example, TCRβV is present in about 3-6% of tumor infiltrating T cells irrespective of tumor type (see Li B. et al., Nature Genetics, 2016, vol:48(7):725-32 the entire contents of which are hereby incorporated by references). Li et al., also disclose that TCRβ V6-5 is present at a high frequency in tumor cells.

### Anti-TCRβ V6 Antibodies

In one aspect, provided herein is an anti-TCRβV antibody molecule that binds to human TCRβ V6, e.g., a TCRβ V6 subfamily comprising: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments the TCRβ V6 subfamily comprises TCRβ V6-5*01 or a variant thereof. In some embodiments, TCRβ V6 comprises TCRβ V6-4*01, or a variant thereof. In some embodiments, TCRβ V6 comprises TCRβ V6-4*02, or a variant thereof. In some embodiments, TCRβ V6 comprises TCRβ V6-9*01, or a variant thereof. In some embodiments, TCRβ V6 comprises TCRβ V6-8*01, or a variant thereof. In some embodiments, TCRβ V6 comprises TCRβ V6-5*01, or a variant thereof. In some embodiments, TCRβ V6 comprises TCRβ V6-6*02, or a variant thereof. In some embodiments, TCRβ V6 comprises TCRβ V6-6*01, or a variant thereof. In some embodiments, TCRβ V6 comprises TCRβ V6-2*01, or a variant thereof. In some embodiments, TCRβ V6 comprises TCRβ V6-3*01, or a variant thereof. In some embodiments, TCRβ V6 comprises TCRβ V6-1*01, or a variant thereof.

In some embodiments, TCRβ V6-5*01 is encoded by the nucleic acid sequence of SEQ ID NO: 43, or a sequence having 85%, 90%, 95%, 99% or more identity thereof. In some embodiments, TCRβ V6-5*01 comprises the amino acid sequence of SEQ ID NO: 44, or an amino acid sequence having 85%, 90%, 95%, 99% or more identity thereof.

In some embodiments, the anti-TCRβV antibody molecule, e.g., anti-TCRβ V6 (e.g., anti-TCRβ V6-5*01) antibody molecule, is a non-murine antibody molecule, e.g., a human or humanized antibody molecule. In some embodiments, the anti-TCRβV antibody molecule, e.g., anti-TCRβ V6 (e.g., anti-TCRβ V6-5*01) antibody molecule is a human antibody molecule. In some embodiments, the anti-TCRβV antibody molecule, e.g., anti-TCRβ V6 (e.g., anti-TCRβ V6-5*01) antibody molecule is a humanized antibody molecule.

In some embodiments, the anti-TCRβV antibody molecule, e.g., anti-TCRβ V6 (e.g., anti-TCRβ V6-5*01) antibody molecule, is isolated or recombinant.

In some embodiments, the anti-TCRβV antibody molecule, e.g., anti-TCRβ V6 (e.g., anti-TCRβ V6-5*01) antibody molecule, comprises at least one antigen-binding region, e.g., a variable region or an antigen-binding fragment thereof, from an antibody described herein, e.g., an antibody chosen from any one of A-H.1 to A-H.85, e.g., A-H.1, A-H.2 or A-H.68, or an antibody described in Table 1, or encoded by a nucleotide sequence in Table 1, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises at least one, two, three or four variable regions from an antibody described herein, e.g., an antibody chosen from any one of A-H.1 to A-H.85, e.g., A-H.1, A-H.2 or A-H.68, or an antibody described in Table 1, Table 43, Table 23A or Table 23B, or encoded by a nucleotide sequence in Table 1, Table 16, Table 23A, or Table 23B, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises at least one or two heavy chain variable regions from an antibody described herein, e.g., an antibody chosen from any one of A-H.1 to A-H.85, e.g., A-H.1, A-H.2 or A-H.68, or an antibody molecule described in Table 30, Table 43A, or Table 43B, or encoded by a nucleotide sequence in Table 30, Table 16, Table 23A, or Table 23B, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule comprises a heavy chain variable region (VH) having a consensus sequence of SEQ ID NO: 8682 or 3290.

Consensus VH
SEQ ID NO: 8682
QVQLVQSGAEVKKPGSSVKVSCKASGH/T/G/YD/T/SFH/R/D/
K/TL/D/K/T/NW/F/T/I/Y/GYIHWVRQAPGQGLEWMGR/WV/
I/FF/S/YA/PGSGN/ST/V/Y/IK/RYNEKFKGRVTITADTSTS
TAYMELSSLRSEDTAVYYCAG/VSY/IYSY/AD/GVLDYWGQGTT
VTVSS

SEQ ID NO: 3290—Consensus VH QVQLVQSGAEVKKPGSSVKVSCKASGX₁X₂FX₃X₄X₅YIHWVRQAPGQGLEWMGX₆X₇X₈X₉GSGX ₁₀X₁₁X₁₂YNEKFKGRVTITADTSTSTAYMELSSLRSEDTAVYYCAX₁₃SX₁₄YSX₁₅X₁₆VLDYWGQGT TVTVSS, where-in: X1 is H or T or G or Y; X2 is D or T or S; X3 is H or R or D or K or T; X4 is L or D or K or T or N; X5 is W or F or T or I or Y or G; X6 is R or W; X7 is V or I or F; X8 is F or S or Y; X9 is A or P; X10 is N or S; X11 is T or V or Y or I; X12 is K or R; X13 is G or V; X14 is Y or I; X15 is Y or A; and X16 is D or G.

In some embodiments, the anti-TCRβV antibody molecule, e.g., anti-TCRβ V6 (e.g., anti-TCRβ V6-5*01) antibody molecule, comprises at least one or two light chain variable regions from an antibody described herein, e.g., an antibody chosen from any one of A-H.1 to A-H.85, e.g., A-H.1, A-H.2 or A-H.68, or an antibody described in Table 30, Table 43A, or Table 43B, or encoded by a nucleotide sequence in Table 30, Table 43A, or Table 43B, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule comprises a light chain variable region (VL) having a consensus sequence of SEQ ID NO: 8681 or 3289.

Consensus VL
SEQ ID NO: 8681
DIQMTQSPSFLSASVGDRVTITCKASQNVG/E/A/DN/DR/KVAW
Y/HQQKPGKAPKALIYSSSHRYK/SGVPSRFSGSGSGTEFTLTIS
SLQPEDFATYFCQQFKSYPLTFGQGTKLEIK

SEQ ID NO: 3289—Consensus VL DIQMTQSPSFLSASVGDRVTITCKASQNVX₁X₂X₃VAWX₄QQKPGKAPKALIYSSSHRYXsGVPSRF SGSGSGTEFTLTISSLQPEDFATYFCQQFKSYPLTFGQGTKLEIK, wherein X1 is G, E, A or D; X2 is N or D; X3 is R or K; X4 is Y or H; and X5 is K or S

In some embodiments, the anti-TCRβV antibody molecule, e.g., anti-TCRβ V6 (e.g., anti-TCRβ V6-5*01) antibody molecule, comprises a heavy chain constant region for an IgG4, e.g., a human IgG4. In still another embodiment, the anti-TCRβV antibody molecule, e.g., anti-TCRβ V6 (e.g., anti-TCRβ V6-5*01) antibody molecule includes a heavy chain constant region for an IgG1, e.g., a human IgG1. In some embodiments, the heavy chain constant region comprises an amino sequence set forth in Table 3, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V6 (e.g., anti-TCRβ V6-5*01) antibody molecule, includes a kappa light chain constant region, e.g., a human kappa light chain constant region. In some embodiments, the light chain constant region comprises an amino sequence set forth in Table 3, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V6 (e.g., anti-TCRβ V6-5*01) antibody molecule, includes at least one, two, or three complementarity determining regions (CDRs) from a heavy chain variable region (VH) of an antibody described herein, e.g., an antibody chosen from any one of A-H.1 to A-H.85, e.g., A-H.1, A-H.2 or A-H.68, or an antibody described in Table 30, Table 43A, or Table 43B, or encoded by a nucleotide sequence in Table 30, Table 43A, or Table 43B, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, includes at least one, two, or three CDRs (or collectively all of the CDRs) from a heavy chain variable region comprising an amino acid sequence shown in Table 30, Table 43A, or Table 43B, or encoded by a nucleotide sequence shown in Table 30, Table 43A, or Table 43B. In some embodiments, one or more of the CDRs (or collectively all of the CDRs) have one, two, three, four, five, six or more changes, e.g., amino acid substitutions or deletions, relative to the amino acid sequence shown in Table 30, Table 43A, or Table 43B, or encoded by a nucleotide sequence shown in Table 30, Table 43A, or Table 43B.

In some embodiments, the anti-TCRβV antibody molecule, e.g., anti-TCRβ V6 (e.g., anti-TCRβ V6-5*01) antibody molecule, includes at least one, two, or three complementarity determining regions (CDRs) from a light chain variable region of an antibody described herein, e.g., an antibody chosen from any one of A-H.1 to A-H.85, e.g., A-H.1, A-H.2 or A-H.68, or an antibody described in Table 30, Table 43A, or Table 43B, or encoded by a nucleotide sequence in Table 30, Table 43A, or Table 43B, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, includes at least one, two, or three CDRs (or collectively all of the CDRs) from a light chain variable region comprising an amino acid sequence shown in Table 30, Table 43A, or Table 43B, or encoded by a nucleotide sequence shown in Table 30, Table 43A, or Table 43B. In some embodiments, one or more of the CDRs (or collectively all of the CDRs) have one, two, three, four, five, six or more changes, e.g., amino acid substitutions or deletions, relative to the amino acid sequence shown in Table 30, Table 43A, or Table 43B, or encoded by a nucleotide sequence shown in Table 30, Table 43A, or Table 43B.

In some embodiments, the anti-TCRβV antibody molecule, e.g., anti-TCRβ V6 (e.g., anti-TCRβ V6-5*01) antibody molecule, includes at least one, two, three, four, five or six CDRs (or collectively all of the CDRs) from a heavy and light chain variable region comprising an amino acid sequence shown in Table 30, Table 43A, or Table 43B, or encoded by a nucleotide sequence shown in Table 30, Table 43A, or Table 43B. In some embodiments, one or more of the CDRs (or collectively all of the CDRs) have one, two, three, four, five, six or more changes, e.g., amino acid substitutions or deletions, relative to the amino acid sequence shown in Table 30, Table 43A, or Table 43B, or encoded by a nucleotide sequence shown in Table 30, Table 43A, or Table 43B.

In some embodiments, the anti-TCRβV antibody molecule, e.g., anti-TCRβ V6 (e.g., anti-TCRβ V6-5*01) antibody molecule, molecule includes all six CDRs from an antibody described herein, e.g., an antibody chosen from any one of A-H.1 to A-H.85, e.g., A-H.1, A-H.2 or A-H.68, or an antibody described in Table 30, Table 43A, or Table 43B, or encoded by a nucleotide sequence in Table 30, Table 43A, or Table 43B, or closely related CDRs, e.g., CDRs which are identical or which have at least one amino acid alteration, but not more than two, three or four alterations (e.g., substitutions, deletions, or insertions, e.g., conservative substitutions). In some embodiments, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule includes at least one, two, or three CDRs according to Kabat et al. (e.g., at least one, two, or three CDRs according to the Kabat definition as set out in Table 30, Table 43A, or Table 43B) from a heavy chain variable region of an antibody described herein, e.g., an antibody chosen from any one of A-H.1 to A-H.85, e.g., A-H.1, A-H.2 or A-H.68, or an antibody described in Table 30, Table 43A, or Table 43B, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 30, Table 43A, or Table 43B.

In some embodiments, the anti-TCRβV antibody molecule, e.g., anti-TCRβ V6 (e.g., anti-TCRβ V6-5*01) antibody molecule includes at least one, two, or three CDRs according to Kabat et al. (e.g., at least one, two, or three CDRs according to the Kabat definition as set out in Table 30, Table 43A, or Table 43B) from a light chain variable region of an antibody described herein, e.g., an antibody chosen from any one of A-H.1 to A-H.85, e.g., A-H.1, A-H.2 or A-H.68, or an antibody described in Table 30, Table 43A, or Table 43B, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 30, Table 43A, or Table 43B.

In some embodiments, the anti-TCRβV antibody molecule, e.g., anti-TCRβ V6 (e.g., anti-TCRβ V6-5*01) antibody molecule, includes at least one, two, three, four, five, or six CDRs according to Kabat et al. (e.g., at least one, two, three, four, five, or six CDRs according to the Kabat definition as set out in Table 30, Table 43A, or Table 43B) from the heavy and light chain variable regions of an antibody described herein, e.g., an antibody chosen from any one of A-H.1 to A-H.85, e.g., A-H.1, A-H.2 or A-H.68, or an antibody described in Table 30, Table 43A, or Table 43B, Table 23A or Table 23B, or encoded by a nucleotide sequence in Table 30, Table 43A, or Table 43B; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Table 30, Table 43A, or Table 43B.

In some embodiments, the anti-TCRβV antibody molecule, e.g., anti-TCRβ V6 (e.g., anti-TCRβ V6-5*01) antibody molecule, includes all six CDRs according to Kabat et al. (e.g., all six CDRs according to the Kabat definition as set out in Table 30, Table 43A, or Table 43B from the heavy and light chain variable regions of an antibody described herein, e.g., an antibody chosen from any one of A-H.1 to A-H.85, e.g., A-H.1, A-H.2 or A-H.68, or an antibody described in Table 30, Table 43A, or Table 43B, or encoded by a nucleotide sequence in Table 30, Table 43A, or Table 43B; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 30, Table 43A, or Table 43B. In some embodiments, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule, includes at least one, two, or three hypervariable loops that have the same canonical structures as the corresponding hypervariable loop of an antibody described herein, e.g., an antibody chosen from chosen from any one of A-H.1 to A-H.85, e.g., A-H.1, A-H.2 or A-H.68, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In some embodiments, the anti-TCRβV antibody molecule, e.g., anti-TCRβ V6 (e.g., anti-TCRβ V6-5*01) antibody molecule includes at least one, two, or three CDRs according to Chothia et al. (e.g., at least one, two, or three CDRs according to the Chothia definition as set out in Table 30, Table 43A, or Table 43B) from a heavy chain variable region of an antibody described herein, e.g., an antibody chosen from any one of A-H.1 to A-H.85, e.g., A-H.1, A-H.2 or A-H.68, or as described in Table 30, Table 43A, or Table 43B, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 30, Table 43A, or Table 43B.

In some embodiments, the anti-TCRβV antibody molecule, e.g., anti-TCRβ V6 (e.g., anti-TCRβ V6-5*01) antibody molecule includes at least one, two, or three CDRs according to Chothia et al. (e.g., at least one, two, or three CDRs according to the Chothia definition as set out in Table 30, Table 43A, or Table 43B) from a light chain variable region of an antibody described herein, e.g., an antibody chosen from any one of A-H.1 to A-H.85, e.g., A-H.1, A-H.2 or A-H.68, or an antibody described in Table 30, Table 43A, or Table 43B, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 30, Table 43A, or Table 43B.

In some embodiments, the anti-TCRβV antibody molecule, e.g., anti-TCRβ V6 (e.g., anti-TCRβ V6-5*01) antibody molecule, includes at least one, two, three, four, five, or six CDRs according to Chothia et al. (e.g., at least one, two, three, four, five, or six CDRs according to the Chothia definition as set out in Table 30, Table 43A, or Table 43B) from the heavy and light chain variable regions of an antibody described herein, e.g., an antibody chosen from any one of A-H.1 to A-H.85, e.g., A-H.1, A-H.2 or A-H.68, or an antibody described in Table 30, Table 43A, or Table 43B, or encoded by the nucleotide sequence in Table 30, Table 43A, or Table 43B; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Chothia et al. shown in Table 30, Table 43A, or Table 43B.

In some embodiments, the anti-TCRβV antibody molecule, e.g., anti-TCRβ V6 (e.g., anti-TCRβ V6-5*01) antibody molecule, includes all six CDRs according to Chothia et al. (e.g., all six CDRs according to the Chothia definition as set out in Table 30, Table 43A, or Table 43B) from the heavy and light chain variable regions of an antibody described herein, e.g., an antibody chosen from any one of A-H.1 to A-H.85, e.g., A-H.1, A-H.2 or A-H.68, or an antibody described in Table 30, Table 43A, or Table 43B, or encoded by a nucleotide sequence in Table 30, Table 43A, or Table 43B; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Chothia et al. shown in Table 30, Table 43A, or Table 43B. In some embodiments, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule, molecule includes a combination of CDRs or hypervariable loops defined according to Kabat et al., Chothia et al., ImMunoGeneTics (IMGT) numbering system, or as described in Table 30, Table 43A, or Table 43B.

In some embodiments, the anti-TCRβV antibody molecule, e.g., anti-TCRβ V6 (e.g., anti-TCRβ V6-5*01) antibody molecule, can contain any combination of CDRs or hypervariable loops according to the Kabat and Chothia definitions or by ImMunoGeneTics (IMGT) numbering system.

In some embodiments, a combined CDR as set out in Table 30, Table 43A, or Table 43B is a CDR that comprises a Kabat CDR and a Chothia CDR.

In some embodiments, the anti-TCRβV antibody molecule, e.g., anti-TCRβ V6 (e.g., anti-TCRβ V6-5*01) antibody molecule, molecule includes a combination of CDRs or hypervariable loops identified as combined CDRs in Table 30, Table 43A, or Table 43B. In some embodiments, the anti-TCRβV antibody molecule, e.g., anti-TCRβ V6 (e.g., anti-TCRβ V6-5*01) antibody molecule, can contain any combination of CDRs or hypervariable loops according the “combined” CDRs are described in Table 30, Table 43A, or Table 43B.

In some embodiments, e.g., an embodiment comprising a variable region, a CDR (e.g., a combined CDR, Chothia CDR or Kabat CDR, or a CDR by ImMunoGeneTics (IMGT) numbering system), or other sequence referred to herein, e.g., in Table 30, Table 43A, or Table 43B, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multispecific molecule, e.g., a bispecific molecule, e.g., as described herein.

In some embodiments, the anti-TCRβV antibody molecule, e.g., anti-TCRβ V6 (e.g., anti-TCRβ V6-5*01) antibody molecule includes: (i) one, two or all of a light chain complementarity determining region 1 (LC CDR1), a light chain complementarity determining region 2 (LC CDR2), and a light chain complementarity determining region 3 (LC CDR3) of SEQ ID NO: 2, SEQ ID NO: 10 or SEQ ID NO: 11, and/or (ii) one, two or all of a heavy chain complementarity determining region 1 (HC CDR1), heavy chain complementarity determining region 2 (HC CDR2), and a heavy chain complementarity determining region 3 (HC CDR3) of SEQ ID NO: 1 or SEQ ID NO: 9.

In some embodiments, the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 2, and a HC CDR1, HC CDR2, and HC CDR3 of SEQ ID NO: 1.

In some embodiments the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 10, and a HC CDR1, HC CDR2, and HC CDR3 of SEQ ID NO: 9.

In some embodiments, the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 11, and a HC CDR1, HC CDR2, and HC CDR3 of SEQ ID NO: 9.

In some embodiments, the anti-TCRβV antibody molecule, e.g., anti-TCRβ V6 (e.g., anti-TCRβ V6-5*01) antibody molecule comprises: (i) a LC CDR1 amino acid sequence of SEQ ID NO: 6, a LC CDR2 amino acid sequence of SEQ ID NO: 7, or a LC CDR3 amino acid sequence of SEQ ID NO: 8; and/or (ii) a HC CDR1 amino acid sequence of SEQ ID NO: 3, a HC CDR2 amino acid sequence of SEQ ID NO: 4, or a HC CDR3 amino acid sequence of SEQ ID NO: 5.

In some embodiments, the anti-TCRβV antibody molecule, e.g., anti-TCRβ V6 (e.g., anti-TCRβ V6-5*01) antibody molecule comprises: (i) a light chain variable region (VL) comprising a LC CDR1 amino acid sequence of SEQ ID NO: 6, a LC CDR2 amino acid sequence of SEQ ID NO: 7, or a LC CDR3 amino acid sequence of SEQ ID NO: 8; and/or (ii) a heavy chain variable region (VH) comprising a HC CDR1 amino acid sequence of SEQ ID NO: 3, a HC CDR2 amino acid sequence of SEQ ID NO: 4, or a HC CDR3 amino acid sequence of SEQ ID NO: 5.

In some embodiments, the anti-TCRβV antibody molecule, e.g., anti-TCRβ V6 (e.g., anti-TCRβ V6-5*01) antibody molecule comprises: (i) a LC CDR1 amino acid sequence of SEQ ID NO: 51, a LC CDR2 amino acid sequence of SEQ ID NO: 52, or a LC CDR3 amino acid sequence of SEQ ID NO: 53; and/or (ii) a HC CDR1 amino acid sequence of SEQ ID NO: 45, a HC CDR2 amino acid sequence of SEQ ID NO: 46, or a HC CDR3 amino acid sequence of SEQ ID NO: 47.

In some embodiments, the anti-TCRβV antibody molecule, e.g., anti-TCRβ V6 (e.g., anti-TCRβ V6-5*01) antibody molecule comprises: (i) a light chain variable region (VL) comprising a LC CDR1 amino acid sequence of SEQ ID NO: 51, a LC CDR2 amino acid sequence of SEQ ID NO: 52, or a LC CDR3 amino acid sequence of SEQ ID NO: 53; and/or (ii) a heavy chain variable region (VH) comprising a HC CDR1 amino acid sequence of SEQ ID NO: 45, a HC CDR2 amino acid sequence of SEQ ID NO: 46, or a HC CDR3 amino acid sequence of SEQ ID NO: 47.

In some embodiments, the anti-TCRβV antibody molecule, e.g., anti-TCRβ V6 (e.g., anti-TCRβ V6-5*01) antibody molecule comprises: (i) a LC CDR1 amino acid sequence of SEQ ID NO: 54, a LC CDR2 amino acid sequence of SEQ ID NO: 55, or a LC CDR3 amino acid sequence of SEQ ID NO: 56; and/or (ii) a HC CDR1 amino acid sequence of SEQ ID NO: 48, a HC CDR2 amino acid sequence of SEQ ID NO: 49, or a HC CDR3 amino acid sequence of SEQ ID NO: 50.

In some embodiments, the anti-TCRβV antibody molecule, e.g., anti-TCRβ V6 (e.g., anti-TCRβ V6-5*01) antibody molecule comprises: (i) a light chain variable region (VL) comprising a LC CDR1 amino acid sequence of SEQ ID NO: 54, a LC CDR2 amino acid sequence of SEQ ID NO: 55, or a LC CDR3 amino acid sequence of SEQ ID NO: 56; and/or (ii) a heavy chain variable region (VH) comprising a HC CDR1 amino acid sequence of SEQ ID NO: 48, a HC CDR2 amino acid sequence of SEQ ID NO: 49, or a HC CDR3 amino acid sequence of SEQ ID NO: 50.

In some embodiments, the anti-TCRβV antibody molecule, e.g., anti-TCRβ V6 (e.g., anti-TCRβ V6-5*01) antibody molecule comprises a VH and/or a VL of an antibody described in Table 30, Table 43A, or Table 43B, or a sequence with at least 80%, 85%, 90%, 95%, 96%, 97%, 98%, 99% or more identity thereto.

In some embodiments, the anti-TCRβV antibody molecule, e.g., anti-TCRβ V6 (e.g., anti-TCRβ V6-5*01) antibody molecule comprises a VH and a VL of an antibody described in Table 30, Table 43A, or Table 43B, or a sequence with at least 80%, 85%, 90%, 95%, 96%, 97%, 98%, 99% or more identity thereto.

In some embodiments, an anti-TCRVb antibody as described herein has an antigen binding domain having a VL having a consensus sequence of SEQ ID NO: 8681, wherein position 30 is G, E, A or D; position 31 is N or D; position 32 is R or K; position 36 is Y or H; and/or position 56 is K or S.

In some embodiments, an anti-TCRVb antibody as described herein has an antigen binding domain having a VH having a consensus sequence of SEQ ID NO: 8682, wherein: position 27 is H or T or G or Y; position 28 is D or T or S; position 30 is H or R or D or K or T; position 31 is L or D or K or T or N; position 32 is W or F or T or I or Y or G; position 49 is R or W; position 50 is V or I or F; position 51 is F or S or Y; position 52 is A or P; position 56 is N or S; position 57 is T or V or Y or I; position 58 is K or R; position 97 is G or V; position 99 is Y or I; position 102 is Y or A; and/or position 103 is D or G.

### Additional Anti-TCRVβ Antibodies

Additional exemplary anti-TCRβV antibodies are provided in Table 32, Table 42B, Table 42C, Table 43, Table 44, Table 45, Table 46, Table 47, Table 48, Table 49, Table 50, and Table 51. In some embodiments, the anti-TCRβV antibody is a humanized antibody, e.g., as provided in Table 32, Table 42B, Table 42C, Table 43, Table 44, Table 45, Table 46, Table 47, Table 48, Table 49, Table 50, and Table 51. In some embodiments, the anti-TCRβV antibody comprises one or more (e.g., all three) of a LC CDR1, LC CDR2, and LC CDR3 provided in Table 2, Table 42B, Table 42C, Table 43, Table 44, Table 45, Table 46, Table 47, Table 48, Table 49, Table 50, and Table 51; and/or one or more (e.g., all three) of a HC CDR1, HC CDR2, and HC CDR3 provided in Table 2, Table 42B, Table 42C, Table 43, Table 44, Table 45, Table 46, Table 47, Table 48, Table 49, Table 50, and Table 51, or a sequence with at least 95% sequence identity thereto. In some embodiments, the anti-TCRβV antibody comprises a variable heavy chain (VH) and/or a variable light chain (VL) provided in Table 45, or a sequence with at least 95% sequence identity thereto.

### B Cell, Macrophage & Dendritic Cell Engagers

Broadly, B cells, also known as B lymphocytes, are a type of white blood cell of the lymphocyte subtype. They function in the humoral immunity component of the adaptive immune system by secreting antibodies. Additionally, B cells present antigen (they are also classified as professional antigen-presenting cells (APCs)) and secrete cytokines. Macrophages are a type of white blood cell that engulfs and digests cellular debris, foreign substances, microbes, cancer cells via phagocytosis. Besides phagocytosis, they play important roles in nonspecific defense (innate immunity) and also help initiate specific defense mechanisms (adaptive immunity) by recruiting other immune cells such as lymphocytes. For example, they are important as antigen presenters to T cells. Beyond increasing inflammation and stimulating the immune system, macrophages also play an important anti-inflammatory role and can decrease immune reactions through the release of cytokines. Dendritic cells (DCs) are antigen-presenting cells that function in processing antigen material and present it on the cell surface to the T cells of the immune system.

The present disclosure provides, inter alia, multispecific (e.g., bi-, tri-, quad-specific) or multifunctional molecules, that include, e.g., are engineered to contain, one or more B cell, macrophage, and/or dendritic cell engager that mediate binding to and/or activation of a B cell, macrophage, and/or dendritic cell.

Accordingly, in some embodiments, the immune cell engager comprises a B cell, macrophage, and/or dendritic cell engager chosen from one or more of CD40 ligand (CD40L) or a CD70 ligand; an antibody molecule that binds to CD40 or CD70; an antibody molecule to OX40; an OX40 ligand (OX40L); an agonist of a Toll-like receptor (e.g., as described herein, e.g., a TLR4, e.g., a constitutively active TLR4 (caTLR4), or a TLR9 agonists); a 41BB; a CD2; a CD47; or a STING agonist, or any combination thereof.

In some embodiments, the B cell engager is a CD40L, an OX40L, or a CD70 ligand, or an antibody molecule that binds to OX40, CD40 or CD70.

In some embodiments, the macrophage engager is a CD2 agonist. In some embodiments, the macrophage engager is an antigen binding domain that binds to: CD40L or antigen binding domain or ligand that binds CD40, a Toll like receptor (TLR) agonist (e.g., as described herein), e.g., a TLR9 or TLR4 (e.g., caTLR4 (constitutively active TLR4), CD47, or a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In some embodiments, the dendritic cell engager is a CD2 agonist. In some embodiments, the dendritic cell engager is a ligand, a receptor agonist, or an antibody molecule that binds to one or more of: OX40L, 41BB, a TLR agonist (e.g., as described herein) (e.g., TLR9 agonist, TLR4 (e.g., caTLR4 (constitutively active TLR4)), CD47, or and a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In other embodiments, the immune cell engager mediates binding to, or activation of, one or more of a B cell, a macrophage, and/or a dendritic cell. Exemplary B cell, macrophage, and/or dendritic cell engagers can be chosen from one or more of CD40 ligand (CD40L) or a CD70 ligand; an antibody molecule that binds to CD40 or CD70; an antibody molecule to OX40; an OX40 ligand (OX40L); a Toll-like receptor agonist (e.g., a TLR4, e.g., a constitutively active TLR4 (caTLR4) or a TLR9 agonist); a 41BB agonist; a CD2; a CD47; or a STING agonist, or any combination thereof.

In some embodiments, the B cell engager is chosen from one or more of a CD40L, an OX40L, or a CD70 ligand, or an antibody molecule that binds to OX40, CD40 or CD70.

In other embodiments, the macrophage cell engager is chosen from one or more of a CD2 agonist; a CD40L; an OX40L; an antibody molecule that binds to OX40, CD40 or CD70; a Toll-like receptor agonist or a fragment thereof (e.g., a TLR4, e.g., a constitutively active TLR4 (caTLR4)); a CD47 agonist; or a STING agonist.

In other embodiments, the dendritic cell engager is chosen from one or more of a CD2 agonist, an OX40 antibody, an OX40L, 41BB agonist, a Toll-like receptor agonist or a fragment thereof (e.g., a TLR4, e.g., a constitutively active TLR4 (caTLR4)), CD47 agonist, or a STING agonist.

In one embodiment, the OX40L comprises the amino acid sequence:

QVSHRYPRIQSIKVQFTEYKKEKGFILTSQKEDEIMKVQNNSVIINCDGFYLISLKGYFSQE VNISLHYQKDEEPLFQLKKVRSVNSLMVASLTYKDKVYLNVTTDNTSLDDFHVNGGELILIHQN PGEFCVL (SEQ ID NO: 7245), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7245.

In another embodiment, the CD40L comprises the amino acid sequence: MQKGDQNPQIAAHVISEASSKT TSVLQWAEKGYYTMSNNLVTLENGKQLTVKRQGLYYIYAQ VTFCSNREASSQAPFIASLCLKSPGRFERILLRAANTHSSAKPCGQQSIHLGGVFELQPGASVFVN VTDPSQVSHGTGFTSFGLLKL (SEQ ID NO: 7246), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7246.

In yet other embodiments, the STING agonist comprises a cyclic dinucleotide, e.g., a cyclic di-GMP (cdGMP), a cyclic di-AMP (cdAMP), or any combination thereof, optionally with 2′,5′ or 3′,5′ phosphate linkages.

In one embodiment, the immune cell engager includes 41BB ligand, e.g., comprising the amino acid sequence: ACPWAVSGARASPGSAASPRLREGPELSPDDPAGLLDLRQGMFAQLVAQNVLLIDGPLSWYSDP GLAGVSLTGGLSYKEDTKELVVAKAGVYYVFFQLELRRVVAGEGSGSVSLALHLQPLRSAAGA AALALTVDLPPASSEARNSAFGFQGRLLHLSAGQRLGVHLHTEARARHAWQLTQGATVLGLFR VTPEIPAGLPSPRSE (SEQ ID NO: 7247), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7247.

### Toll-Like Receptors

Toll-Like Receptors (TLRs) are evolutionarily conserved receptors are homologues of the Drosophila Toll protein, and recognize highly conserved structural motifs known as pathogen-associated microbial patterns (PAMPs), which are exclusively expressed by microbial pathogens, or danger-associated molecular patterns (DAMPs) that are endogenous molecules released from necrotic or dying cells. PAMPs include various bacterial cell wall components such as lipopolysaccharide (LPS), peptidoglycan (PGN) and lipopeptides, as well as flagellin, bacterial DNA and viral double-stranded RNA. DAMPs include intracellular proteins such as heat shock proteins as well as protein fragments from the extracellular matrix. Stimulation of TLRs by the corresponding PAMPs or DAMPs initiates signaling cascades leading to the activation of transcription factors, such as AP-1, NF-xB and interferon regulatory factors (IRFs). Signaling by TLRs results in a variety of cellular responses, including the production of interferons (IFNs), pro-inflammatory cytokines and effector cytokines that direct the adaptive immune response. TLRs are implicated in a number of inflammatory and immune disorders and play a role in cancer (Rakoff-Nahoum S. & Medzhitov R., 2009. Toll-like receptors and cancer. Nat Revs Cancer 9:57-63.)

TLRs are type I transmembrane proteins characterized by an extracellular domain containing leucine-rich repeats (LRRs) and a cytoplasmic tail that contains a conserved region called the Toll/IL-1 receptor (TIR) domain. Ten human and twelve murine TLRs have been characterized, TLR1 to TLR10 in humans, and TLR1 to TLR9, TLR11, TLR12 and TLR13 in mice, the homolog of TLR10 being a pseudogene. TLR2 is essential for the recognition of a variety of PAMPs from Gram-positive bacteria, including bacterial lipoproteins, lipomannans and lipoteichoic acids. TLR3 is implicated in virus-derived double-stranded RNA. TLR4 is predominantly activated by lipopolysaccharide. TLR5 detects bacterial flagellin and TLR9 is required for response to unmethylated CpG DNA. Finally, TLR7 and TLR8 recognize small synthetic antiviral molecules, and single-stranded RNA was reported to be their natural ligand. TLR11 has been reported to recognize uropathogenic E. coli and a profilin-like protein from Toxoplasma gondii. The repertoire of specificities of the TLRs is apparently extended by the ability of TLRs to heterodimerize with one another. For example, dimers of TLR2 and TLR6 are required for responses to diacylated lipoproteins while TLR2 and TLR1 interact to recognize triacylated lipoproteins. Specificities of the TLRs are also influenced by various adapter and accessory molecules, such as MD-2 and CD14 that form a complex with TLR4 in response to LPS.

TLR signaling consists of at least two distinct pathways: a MyD88-dependent pathway that leads to the production of inflammatory cytokines, and a MyD88-independent pathway associated with the stimulation of IFN-β and the maturation of dendritic cells. The MyD88-dependent pathway is common to all TLRs, except TLR3 (Adachi O. et al., 1998. Targeted disruption of the MyD88 gene results in loss of IL-1- and IL-18-mediated function. Immunity. 9(1):143-50). Upon activation by PAMPs or DAMPs, TLRs hetero- or homodimerize inducing the recruitment of adaptor proteins via the cytoplasmic TIR domain. Individual TLRs induce different signaling responses by usage of the different adaptor molecules. TLR4 and TLR2 signaling requires the adaptor TIRAP/Mal, which is involved in the MyD88-dependent pathway. TLR3 triggers the production of IFN-β in response to double-stranded RNA, in a MyD88-independent manner, through the adaptor TRIF/TICAM-1. TRAM/TICAM-2 is another adaptor molecule involved in the MyD88-independent pathway which function is restricted to the TLR4 pathway.

TLR3, TLR7, TLR8 and TLR9 recognize viral nucleic acids and induce type I IFNs. The signaling mechanisms leading to the induction of type I IFNs differ depending on the TLR activated. They involve the interferon regulatory factors, IRFs, a family of transcription factors known to play a critical role in antiviral defense, cell growth and immune regulation. Three IRFs (IRF3, IRF5 and IRF7) function as direct transducers of virus-mediated TLR signaling. TLR3 and TLR4 activate IRF3 and IRF7, while TLR7 and TLR8 activate IRF5 and IRF7 (Doyle S. et al., 2002. IRF3 mediates a TLR3/TLR4-specific antiviral gene program. Immunity. 17(3):251-63). Furthermore, type I IFN production stimulated by TLR9 ligand CpG-A has been shown to be mediated by PI(3)K and mTOR (Costa-Mattioli M. & Sonenberg N. 2008. RAPping production of type I interferon in pDCs through mTOR. Nature Immunol. 9: 1097-1099).

### TLR-9

TLR9 recognizes unmethylated CpG sequences in DNA molecules. CpG sites are relatively rare (~1%) on vertebrate genomes in comparison to bacterial genomes or viral DNA. TLR9 is expressed by numerous cells of the immune system such as B lymphocytes, monocytes, natural killer (NK) cells, and plasmacytoid dendritic cells. TLR9 is expressed intracellularly, within the endosomal compartments and functions to alert the immune system of viral and bacterial infections by binding to DNA rich in CpG motifs. TLR9 signals leads to activation of the cells initiating pro-inflammatory reactions that result in the production of cytokines such as type-I interferon and IL-12.

### TLR Agonists

A TLR agonist can agonize one or more TLR, e.g., one or more of human TLR-1, 2, 3, 4, 5, 6, 7, 8, 9, or 10. In some embodiments, an adjunctive agent described herein is a TLR agonist. In some embodiments, the TLR agonist specifically agonizes human TLR-9. In some embodiments, the TLR-9 agonist is a CpG moiety. As used herein, a CpG moiety, is a linear dinucleotide having the sequence: 5′-C-phosphate-G-3′, that is, cytosine and guanine separated by only one phosphate.

In some embodiments, the CpG moiety comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more CpG dinucleotides. In some embodiments, the CpG moiety consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 CpG dinucleotides. In some embodiments, the CpG moiety has 1-5, 1-10, 1-20, 1-30, 1-40, 1-50, 5-10, 5-20, 5-30, 10-20, 10-30, 10-40, or 10-50 CpG dinucleotides.

In some embodiments, the TLR-9 agonist is a synthetic ODN (oligodeoxynucleotides). CpG ODNs are short synthetic single-stranded DNA molecules containing unmethylated CpG dinucleotides in particular sequence contexts (CpG motifs). CpG ODNs possess a partially or completely phosphorothioated (PS) backbone, as opposed to the natural phosphodiester (PO) backbone found in genomic bacterial DNA. There are three major classes of CpG ODNs: classes A, B and C, which differ in their immunostimulatory activities. CpG-A ODNs are characterized by a PO central CpG-containing palindromic motif and a PS-modified 3′ poly-G string. They induce high IFN-α production from pDCs but are weak stimulators of TLR9-dependent NF-xB signaling and pro-inflammatory cytokine (e.g. IL-6) production. CpG-B ODNs contain a full PS backbone with one or more CpG dinucleotides. They strongly activate B cells and TLR9-dependent NF-κB signaling but weakly stimulate IFN-α secretion. CpG-C ODNs combine features of both classes A and B. They contain a complete PS backbone and a CpG-containing palindromic motif. C-Class CpG ODNs induce strong IFN-α production from pDC as well as B cell stimulation.

### Stromal Modifying Moieties

Solid tumors have a distinct structure that mimics that of normal tissues and comprises two distinct but interdependent compartments: the parenchyma (neoplastic cells) and the stroma that the neoplastic cells induce and in which they are dispersed. All tumors have stroma and require stroma for nutritional support and for the removal of waste products. In the case of tumors which grow as cell suspensions (e.g., leukemias, ascites tumors), the blood plasma serves as stroma (Connolly J L et al. Tumor Structure and Tumor Stroma Generation. In: Kufe D W et al., editors. Holland-Frei Cancer Medicine. 6^{th} edition. Hamilton: BC Decker; 2003). The stroma includes a variety of cell types, including fibroblasts/myofibroblasts, glial, epithelial, fat, vascular, smooth muscle, and immune cells along with extracellular matrix (ECM) and extracellular molecules (Li Hanchen et al. Tumor Microenvironment: The Role of the Tumor Stroma in Cancer. J of Cellular Biochemistry 101: 805-815 (2007)).

Stromal modifying moieties described herein include moieties (e.g., proteins, e.g., enzymes) capable of degrading a component of the stroma, e.g., an ECM component, e.g., a glycosaminoglycan, e.g., hyaluronan (also known as hyaluronic acid or HA), chondroitin sulfate, chondroitin, dermatan sulfate, heparin sulfate, heparin, entactin, tenascin, aggrecan and keratin sulfate; or an extracellular protein, e.g., collagen, laminin, elastin, fibrinogen, fibronectin, and vitronectin.

### Stromal Modifying Enzymes

In some embodiments, the stromal modifying moiety is an enzyme. For example, the stromal modifying moiety can include, but is not limited to a hyaluronidase, a collagenase, a chondroitinase, a matrix metalloproteinase (e.g., macrophage metalloelastase).

### Hyaluronidases

Hyaluronidases are a group of neutral- and acid-active enzymes found throughout the animal kingdom. Hyaluronidases vary with respect to substrate specificity, and mechanism of action. There are three general classes of hyaluronidases: (1) Mammalian-type hyaluronidases, (EC 3.2.1.35) which are endo-beta-N-acetylhexosaminidases with tetrasaccharides and hexasaccharides as the major end products. They have both hydrolytic and transglycosidase activities, and can degrade hyaluronan and chondroitin sulfates; (2) Bacterial hyaluronidases (EC 4.2.99.1) degrade hyaluronan and, and to various extents, chondroitin sulfate and dermatan sulfate. They are endo-beta-N-acetylhexosaminidases that operate by a beta elimination reaction that yields primarily disaccharide end products; (3) Hyaluronidases (EC 3.2.1.36) from leeches, other parasites, and crustaceans are endo-beta-glucuronidases that generate tetrasaccharide and hexasaccharide end products through hydrolysis of the beta 1-3 linkage.

Mammalian hyaluronidases can be further divided into two groups: (1) neutral active and (2) acid active enzymes. There are six hyaluronidase-like genes in the human genome, HYAL1, HYAL2, HYAL3 HYAL4 HYALP1 and PH20/SPAM1. HYALP1 is a pseudogene, and HYAL3 has not been shown to possess enzyme activity toward any known substrates. HYAL4 is a chondroitinase and lacks activity towards hyaluronan. HYAL1 is the prototypical acid-active enzyme and PH20 is the prototypical neutral-active enzyme. Acid active hyaluronidases, such as HYAL1 and HYAL2 lack catalytic activity at neutral pH. For example, HYAL1 has no catalytic activity in vitro over pH 4.5 (Frost and Stern, “A Microtiter-Based Assay for Hyaluronidase Activity Not Requiring Specialized Reagents”, Analytical Biochemistry, vol. 251, pp. 263-269 (1997). HYAL2 is an acid active enzyme with a very low specific activity in vitro.

In some embodiments the hyaluronidase is a mammalian hyaluronidase. In some embodiments the hyaluronidase is a recombinant human hyaluronidase. In some embodiments, the hyaluronidase is a neutral active hyaluronidase. In some embodiments, the hyaluronidase is a neutral active soluble hyaluronidase. In some embodiments, the hyaluronidase is a recombinant PH20 neutral-active enzyme. In some embodiments, the hyaluronidase is a recombinant PH20 neutral-active soluble enzyme. In some embodiments the hyaluronidase is glycosylated. In some embodiments, the hyaluronidase possesses at least one N-linked glycan. A recombinant hyaluronidase can be produced using conventional methods known to those of skill in the art, e.g., U.S. Pat. No. 7,767,429, the entire contents of which are incorporated by reference herein.

In some embodiments the hyaluronidase is rHuPH20 (also referred to as Hylenex®; presently manufactured by Halozyme; approved by the FDA in 2005 (see e.g., Scodeller P (2014) Hyaluronidase and other Extracellular Matrix Degrading Enzymes for Cancer Therapy: New Uses and Nano-Formulations. J Carcinog Mutage 5:178; U.S. Pat. Nos. 7,767,429; 8,202,517; 7,431,380; 8,450,470; 8,772,246; 8,580,252, the entire contents of each of which is incorporated by reference herein). rHuPH20 is produced by genetically engineered CHO cells containing a DNA plasmid encoding for a soluble fragment of human hyaluronidase PH20. In some embodiments the hyaluronidase is glycosylated. In some embodiments, the hyaluronidase possesses at least one N-linked glycan. A recombinant hyaluronidase can be produced using conventional methods known to those of skill in the art, e.g., U.S. Pat. No. 7,767,429, the entire contents of which are incorporated by reference herein. In some embodiments, rHuPH20 has a sequence at least 95% (e.g., at least 96%, 97%, 98%, 99%, 100%) identical to the amino acid sequence of

(SEQ ID NO: 7248)
LNFRAPPVIPNVPFLWAWNAPSEFCLGKFDEPLDMSLFSFIGSPR
INATGQGVTIFYVDRLGYYPYIDSITGVTVNGGIPQKISLQDHLD
KAKKDITFYMPVDNLGMAVIDWEEWRPTWARNWKPKDVYKNRSIE
LVQQQNVQLSLTEATEKAKQEFEKAGKDFLVETIKLGKLLRPNHL
WGYYLFPDCYNHHYKKPGYNGSCFNVEIKRNDDLSWLWNESTALY
PSIYLNTQQSPVAATLYVRNRVREAIRVSKIPDAKSPLPVFAYTR
IVFTDQVLKFLSQDELVYTFGETVALGASGIVIWGTLSIMRSMKS
CLLLDNYMETILNPYIINVTLAAKMCSQVLCQEQGVCIRKNWNSS
DYLHLNPDNFAIQLEKGGKFTVRGKPTLEDLEQFSEKFYCSCYST
LSCKEKADVKDTDAVDVCIADGVCIDAFLKPPMETEEPQIFYNAS
PSTLS.

In any of the methods provided herein, the anti-hyaluronan agent can be an agent that degrades hyaluronan or can be an agent that inhibits the synthesis of hyaluronan. For example, the anti-hyaluronan agent can be a hyaluronan degrading enzyme. In another example, the anti-hyaluronan agent or is an agent that inhibits hyaluronan synthesis. For example, the anti-hyaluronan agent is an agent that inhibits hyaluronan synthesis such as a sense or antisense nucleic acid molecule against an HA synthase or is a small molecule drug. For example, an anti-hyaluronan agent is 4-methylumbelliferone (MU) or a derivative thereof, or leflunomide or a derivative thereof. Such derivatives include, for example, a derivative of 4-methylumbelliferone (MU) that is 6,7-dihydroxy-4-methyl coumarin or 5,7-dihydroxy-4-methyl coumarin.

In further examples of the methods provided herein, the hyaluronan degrading enzyme is a hyaluronidase. In some examples, the hyaluronan-degrading enzyme is a PH20 hyaluronidase or truncated form thereof to lacking a C-terminal glycosylphosphatidylinositol (GPI) attachment site or a portion of the GPI attachment site. In specific examples, the hyaluronidase is a PH20 selected from a human, monkey, bovine, ovine, rat, mouse or guinea pig PH20. For example, the hyaluronan-degrading enzyme is a human PH20 hyaluronidase that is neutral active and N-glycosylated and is selected from among (a) a hyaluronidase polypeptide that is a full-length PH20 or is a C-terminal truncated form of the PH20, wherein the truncated form includes at least amino acid residues 36-464 of SEQ ID NO: 7248, such as 36-481, 36-482, 36-483, where the full-length PH20 has the sequence of amino acids set forth in SEQ ID NO: 7248; or (b) a hyaluronidase polypeptide comprising a sequence of amino acids having at least 85%, 86%, 87%, 88%, 89%, 90%, 91%, 92%, 93%, 94%, 95%, 96%, 97%, 98%, 99% or more sequence identity with the polypeptide or truncated form of sequence of amino acids set forth in SEQ ID NO: 7248; or (c) a hyaluronidase polypeptide of (a) or (b) comprising amino acid substitutions, whereby the hyaluronidase polypeptide has a sequence of amino acids having at least 85%, 86%, 87%, 88%, 89%, 90%, 91%, 92%, 93%, 94%, 95%, 96%, 97%, 98%, 99% or more sequence identity with the polypeptide set forth in SEQ ID NO: 7248 or the with the corresponding truncated forms thereof. In exemplary examples, the hyaluronan-degrading enzyme is a PH20 that comprises a composition designated rHuPH20.

In other examples, the anti-hyaluronan agent is a hyaluronan degrading enzyme that is modified by conjugation to a polymer. The polymer can be a PEG and the anti-hyaluronan agent a PEGylated hyaluronan degrading enzyme. Hence, in some examples of the methods provided herein the hyaluronan-degrading enzyme is modified by conjugation to a polymer. For example, the hyaluronan-degrading enzyme is conjugated to a PEG, thus the hyaluronan degrading enzyme is PEGylated. In an exemplary example, the hyaluronan-degrading enzyme is a PEGylated PH20 enzyme (PEGPH20). In the methods provided herein, the corticosteroid can be a glucocorticoid that is selected from among cortisones, dexamethasones, hydrocortisones, methylprednisolones, prednisolones and prednisones.

### Chondroitinases

Chondroitinases are enzymes found throughout the animal kingdom which degrade glycosaminoglycans, specifically chondroitins and chondroitin sulfates, through an endoglycosidase reaction. In some embodiments the chondroitinase is a mammalian chondroitinase. In some embodiments the chondroitinase is a recombinant human chondroitinase. In some embodiments the chondroitinase is HYAL4. Other exemplary chondroitinases include chondroitinase ABC (derived from Proteus vulgaris; Japanese Patent Application Laid-open No 6-153947, T. Yamagata et al. J. Biol. Chem., 243, 1523 (1968), S. Suzuki et al, J. Biol. Chem., 243, 1543 (1968)), chondroitinase AC (derived from Flavobacterium heparinum; T. Yamagata et al., J. Biol. Chem., 243, 1523 (1968)), chondroitinase AC II (derived from Arthrobacter aurescens; K. Hiyama, and S. Okada, J. Biol. Chem., 250, 1824 (1975), K. Hiyama and S. Okada, J. Biochem. (Tokyo), 80, 1201 (1976)), Hyaluronidase ACIII (derived from Flavobacterium sp. Hp102; Hirofumi Miyazono et al., Seikagaku, 61, 1023 (1989)), chondroitinase B (derived from Flavobacterium heparinum; Y. M. Michelacci and C. P. Dietrich, Biochem. Biophys. Res. Commun., 56, 973 (1974), Y. M. Michelacci and C. P. Dietrich, Biochem. J., 151, 121 (1975), Kenichi Maeyama et al, Seikagaku, 57, 1189 (1985)), chondroitinase C (derived from Flavobacterium sp. Hp102; Hirofuimi Miyazono et al, Seikagaku, 61, 1023 (1939)), and the like.

### Matrix Metalloproteinases

Matrix metalloproteases (MMPs) are zinc-dependent endopeptidases that are the major proteases involved in extracellular matrix (ECM) degradation. MMPs are capable of degrading a wide range of extracellular molecules and a number of bioactive molecules. Twenty-four MMP genes have been identified in humans, which can be organized into six groups based on domain organization and substrate preference: Collagenases (MMP-1, -8 and -13), Gelatinases (MMP-2 and MMP-9), Stromelysins (MMP-3, -10 and -11), Matrilysin (MMP-7 and MMP-26), Membrane-type (MT)-MMPs (MMP-14, -15, -16, -17, -24 and -25) and others (MMP-12, -19, -20, -21, -23, -27 and -28). In some embodiments, the stromal modifying moiety is a human recombinant MMP (e.g., MMP-1, -2, -3, -4, -5, -6, -7, -8, -9, 10, -11, -12, -13, -14, 15, -15, -17, -18, -19, 20, -21, -22, -23, or -24).

### Collagenases

The three mammalian collagenases (MMP-1, -8, and -13) are the principal secreted endopeptidases capable of cleaving collagenous extracellular matrix. In addition to fibrillar collagens, collagenases can cleave several other matrix and non-matrix proteins including growth factors. Collagenases are synthesized as inactive pro-forms, and once activated, their activity is inhibited by specific tissue inhibitors of metalloproteinases, TIMPs, as well as by non-specific proteinase inhibitors (Ala-aho R et al. Biochimie. Collagenases in cancer. 2005 March-April; 87(3-4):273-86). In some embodiments, the stromal modifying moiety is a collagenase. In some embodiments, the collagenase is a human recombinant collagenase. In some embodiments, the collagenase is MMP-1. In some embodiments, the collagenase is MMP-8. In some embodiments, the collagenase is MMP-13.

### Macrophage Metalloelastase

Macrophage metalloelastase (MME), also known as MMP-12, is a member of the stromelysin subgroup of MMPs and catalyzes the hydrolysis of soluble and insoluble elastin and a broad selection of matrix and nonmatrix substrates including type IV collagen, fibronectin, laminin, vitronectin, entactin, heparan, and chondroitin sulfates (Erja Kerkelä et al. Journal of Investigative Dermatology (2000) 114, 1113-1119; doi:10.1046/j.1523-1747.2000.00993). In some embodiments, the stromal modifying moiety is a MME. In some embodiments, the MME is a human recombinant MME. In some embodiments, the MME is MMP-12.

### Additional Stromal Modifying Moieties

In some embodiments, the stromal modifying moiety causes one or more of: decreases the level or production of a stromal or extracellular matrix (ECM) component; decreases tumor fibrosis; increases interstitial tumor transport; improves tumor perfusion; expands the tumor microvasculature; decreases interstitial fluid pressure (IFP) in a tumor; or decreases or enhances penetration or diffusion of an agent, e.g., a cancer therapeutic or a cellular therapy, into a tumor or tumor vasculature.

In some embodiments, the stromal or ECM component decreased is chosen from a glycosaminoglycan or an extracellular protein, or a combination thereof. In some embodiments, the glycosaminoglycan is chosen from hyaluronan (also known as hyaluronic acid or HA), chondroitin sulfate, chondroitin, dermatan sulfate, heparin, heparin sulfate, entactin, tenascin, aggrecan and keratin sulfate. In some embodiments, the extracellular protein is chosen from collagen, laminin, elastin, fibrinogen, fibronectin, or vitronectin. In some embodiments, the stromal modifying moiety includes an enzyme molecule that degrades a tumor stroma or extracellular matrix (ECM). In some embodiments, the enzyme molecule is chosen from a hyaluronidase molecule, a collagenase molecule, a chondroitinase molecule, a matrix metalloproteinase molecule (e.g., macrophage metalloelastase), or a variant (e.g., a fragment) of any of the aforesaid. The term “enzyme molecule” includes a full length, a fragment or a variant of the enzyme, e.g., an enzyme variant that retains at least one functional property of the naturally occurring enzyme.

In some embodiments, the stromal modifying moiety decreases the level or production of hyaluronic acid. In other embodiments, the stromal modifying moiety comprises a hyaluronan degrading enzyme, an agent that inhibits hyaluronan synthesis, or an antibody molecule against hyaluronic acid.

In some embodiments, the hyaluronan degrading enzyme is a hyaluronidase molecule, e.g., a full length or a variant (e.g., fragment thereof) thereof. In some embodiments, the hyaluronan degrading enzyme is active in neutral or acidic pH, e.g., pH of about 4-5. In some embodiments, the hyaluronidase molecule is a mammalian hyaluronidase molecule, e.g., a recombinant human hyaluronidase molecule, e.g., a full length or a variant (e.g., fragment thereof, e.g., a truncated form) thereof. In some embodiments, the hyaluronidase molecule is chosen from HYAL1, HYAL2, or PH-20/SPAM1, or a variant thereof (e.g., a truncated form thereof). In some embodiments, the truncated form lacks a C-terminal glycosylphosphatidylinositol (GPI) attachment site or a portion of the GPI attachment site. In some embodiments, the hyaluronidase molecule is glycosylated, e.g., comprises at least one N-linked glycan.

In some embodiments, the hyaluronidase molecule comprises the amino acid sequence: LNFRAPPVIPNVPFLWAWNAPSEFCLGKFDEPLDMSLFSFIGSPRINATGQGVTIFYVDR LGYYPYIDSITGVTVNGGIPQKISLQDHLDKAKKDITFYMPVDNLGMAVIDWEEWRPTWARNW KPKDVYKNRSIELVQQQNVQLSLTEATEKAKQEFEKAGKDFLVETIKLGKLLRPNHLWGYYLFP DCYNHHYKKPGYNGSCFNVEIKRNDDLSWLWNESTALYPSIYLNTQQSPVAATLYVRNRVREA IRVSKIPDAKSPLPVFAYTRIVFTDQVLKFLSQDELVYTFGETVALGASGIVIWGTLSIMRSMKSC LLLDNYMETILNPYIINVTLAAKMCSQVLCQEQGVCIRKNWNSSDYLHLNPDNFAIQLEKGGKF TVRGKPTLEDLEQFSEKFYCSCYSTLSCKEKADVKDTDAVDVCIADGVCIDAFLKPPMETEEPQI FYNASPSTLS (SEQ ID NO: 7256), or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7256.

In some embodiments, the hyaluronidase molecule comprises:
(i) the amino acid sequence of 36-464 of SEQ ID NO: 7256;
(ii) the amino acid sequence of 36-481, 36-482, or 36-483 of PH20, wherein PH20 has the sequence of amino acids set forth in SEQ ID NO: 7256; or (iii) an amino acid sequence having at least 95% to 100% sequence identity to the polypeptide or truncated form of sequence of amino acids set forth in SEQ ID NO: 7256; or
(iv) an amino acid sequence having 30, 20, 10, 5 or fewer amino acid substitutions to the amino acid sequence set forth in SEQ ID NO: 7256. In some embodiments, the hyaluronidase molecule comprises an amino acid sequence at least 95% (e.g., at least 95%, 96%, 97%, 98%, 99%, 100%) identical to the amino acid sequence of SEQ ID NO: 7256. In some embodiments, the hyaluronidase molecule is encoded by a nucleotide sequence at least 95% (e.g., at least 96%, 97%, 98%, 99%, 100%) identical to the nucleotide sequence of SEQ ID NO: 7256.

In some embodiments, the hyaluronidase molecule is PH20, e.g., rHuPH20. In some embodiments, the hyaluronidase molecule is HYAL1 and comprises the amino acid sequence: FRGPLLPNRPFTTVWNANTQWCLERHGVDVDVSVFDVVANPGQTFRGPDMTIFYSSQGTYPYY TPTGEPVFGGLPQNASLIAHLARTFQDILAAIPAPDFSGLAVIDWEAWRPRWAFNWDTKDIYRQ RSRALVQAQHPDWPAPQVEAVAQDQFQGAARAWMAGTLQLGRALRPRGLWGFYGFPDCYNY DFLSPNYTGQCPSGIRAQNDQLGWLWGQSRALYPSIYMPAVLEGTGKSQMYVQHRVAEAFRV AVAAGDPNLPVLPYVQIFYDTTNHFLPLDELEHSLGESAAQGAAGVVLWVSWENTRTKESCQAI KEYMDTTLGPFILNVTSGALLCSQALCSGHGRCVRRTSHPKALLLLNPASFSIQLTPGGGPLSLR GALSLEDQAQMAVEFKCRCYPGWQAPWCERKSMW (SEQ ID NO: 7253), or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7253.

In some embodiments, the hyaluronan degrading enzyme, e.g., the hyaluronidase molecule, further comprises a polymer, e.g., is conjugated to a polymer, e.g., PEG. In some embodiments, the hyaluronan-degrading enzyme is a PEGylated PH20 enzyme (PEGPH20). In some embodiments, the hyaluronan degrading enzyme, e.g., the hyaluronidase molecule, further comprises an immunoglobulin chain constant region (e.g., Fc region) chosen from, e.g., the heavy chain constant regions of IgG1, IgG2, IgG3, and IgG4, more particularly, the heavy chain constant region of human IgG1, IgG2, IgG3, or IgG4. In some embodiments, the immunoglobulin constant region (e.g., the Fc region) is linked, e.g., covalently linked to, the hyaluronan degrading enzyme, e.g., the hyaluronidase molecule. In some embodiments, the immunoglobulin chain constant region (e.g., Fc region) is altered, e.g., mutated, to increase or decrease one or more of: Fc receptor binding, antibody glycosylation, the number of cysteine residues, effector cell function, or complement function. In some embodiments, the hyaluronan degrading enzyme, e.g., the hyaluronidase molecule forms a dimer.

In some embodiments, the stromal modifying moiety comprises an inhibitor of the synthesis of hyaluronan, e.g., an HA synthase. In some embodiments, the inhibitor comprises a sense or an antisense nucleic acid molecule against an HA synthase or is a small molecule drug. In some embodiments, the inhibitor is 4-methylumbelliferone (MU) or a derivative thereof (e.g., 6,7-dihydroxy-4-methyl coumarin or 5,7-dihydroxy-4-methyl coumarin), or leflunomide or a derivative thereof.

In some embodiments, the stromal modifying moiety comprises antibody molecule against hyaluronic acid.

In some embodiments, the stromal modifying moiety comprises a collagenase molecule, e.g., a mammalian collagenase molecule, or a variant (e.g., fragment) thereof. In some embodiments, the collagenase molecule is collagenase molecule IV, e.g., comprising the amino acid sequence of: YNFFPRKPKWDKNQITYRIIGYTPDLDPETVDDAFARAFQVWSDVTPLRFSRIHDGEADIMINFG RWEHGDGYPFDGKDGLLAHAFAPGTGVGGDSHFDDDELWTLGEGQVVRVKYGNADGEYCKF PFLFNGKEYNSCTDTGRSDGFLWCSTTYNFEKDGKYGFCPHEALFTMGGNAEGQPCKFPFRFQG TSYDSCTTEGRTDGYRWCGTTEDYDRDKKYGFCPETAMSTVGGNSEGAPCVFPFTFLGNKYES CTSAGRSDGKMWCATTANYDDDRKWGFCPDQGYSLFLVAAHEFGHAMGLEHSQDPGALMAP IYTYTKNFRLSQDDIKGIQELYGASPDIDLGTGPTPTLGPVTPEICKQDIVFDGIAQIRGEIFFFKDR FIWRTVTPRDKPMGPLLVATFWPELPEKIDAVYEAPQEEKAVFFAGNEYWIYSASTLERGYPKPL TSLGLPPDVQRVDAAFNWSKNKKTYIFAGDKFWRYNEVKKKMDPGFPKLIADAWNAIPDNLD AVVDLQGGGHSYFFKGAYYLKLENQSLKSVKFGSIKSDWLGC (SEQ ID NO: 7254), or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 7254.

### Linkers

The multispecific or multifunctional molecule disclosed herein can further include a linker, e.g., a linker between one or more of: the antigen binding domain and the cytokine molecule, the antigen binding domain and the immune cell engager, the antigen binding domain and the stromal modifying moiety, the cytokine molecule and the immune cell engager, the cytokine molecule and the stromal modifying moiety, the immune cell engager and the stromal modifying moiety, the antigen binding domain and the immunoglobulin chain constant region, the cytokine molecule and the immunoglobulin chain constant region, the immune cell engager and the immunoglobulin chain constant region, or the stromal modifying moiety and the immunoglobulin chain constant region. In embodiments, the linker is chosen from: a cleavable linker, a non-cleavable linker, a peptide linker, a flexible linker, a rigid linker, a helical linker, or a non-helical linker, or any combination thereof.

In one embodiment, the multispecific molecule can include one, two, three or four linkers, e.g., a peptide linker. In one embodiment, the peptide linker includes Gly and Ser. In some embodiments, the peptide linker is selected from GGGGS (SEQ ID NO: 7249); GGGGSGGGGS (SEQ ID NO: 7250); GGGGSGGGGSGGGGS (SEQ ID NO: 7251); and DVPSGPGGGGGSGGGGS (SEQ ID NO: 7252). In some embodiments, the peptide linker is a A(EAAAK)nA (SEQ ID NO: 7255) family of linkers (e.g., as described in Protein Eng. (2001) 14 (8): 529-532). These are stiff helical linkers with n ranging from 2
5. In some embodiments, the peptide linker is selected from AEAAAKEAAAKAAA (SEQ ID NO: 302); AEAAAKEAAAKEAAAKAAA (SEQ ID NO: 303); AEAAAKEAAAKEAAAKEAAAKAAA (SEQ ID NO: 8683); and AEAAAKEAAAKEAAAKEAAAKEAAAKAAA(SEQ ID NO: 8684).

### Nucleic Acids

Nucleic acids encoding the aforementioned multispecific or multifunctional molecules are also disclosed.

In certain embodiments, the invention features nucleic acids comprising nucleotide sequences that encode heavy and light chain variable regions and CDRs or hypervariable loops of the antibody molecules, as described herein. For example, the invention features a first and second nucleic acid encoding heavy and light chain variable regions, respectively, of an antibody molecule chosen from one or more of the antibody molecules disclosed herein. The nucleic acid can comprise a nucleotide sequence as set forth in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein.

In certain embodiments, the nucleic acid can comprise a nucleotide sequence encoding at least one, two, or three CDRs or hypervariable loops from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In other embodiments, the nucleic acid can comprise a nucleotide sequence encoding at least one, two, or three CDRs or hypervariable loops from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In yet another embodiment, the nucleic acid can comprise a nucleotide sequence encoding at least one, two, three, four, five, or six CDRs or hypervariable loops from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding at least one, two, or three CDRs or hypervariable loops from a heavy chain variable region having the nucleotide sequence as set forth in the tables herein, a sequence substantially homologous thereto (e.g., a sequence at least about 85%, 90%, 95%, 99% or more identical thereto, and/or capable of hybridizing under the stringency conditions described herein). In another embodiment, the nucleic acid can comprise a nucleotide sequence encoding at least one, two, or three CDRs or hypervariable loops from a light chain variable region having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein). In yet another embodiment, the nucleic acid can comprise a nucleotide sequence encoding at least one, two, three, four, five, or six CDRs or hypervariable loops from heavy and light chain variable regions having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein).

In certain embodiments, the nucleic acid can comprise a nucleotide sequence encoding a cytokine molecule, an immune cell engager, or a stromal modifying moiety disclosed herein.

In another aspect, the application features host cells and vectors containing the nucleic acids described herein. The nucleic acids may be present in a single vector or separate vectors present in the same host cell or separate host cell, as described in more detail hereinbelow.

### Vectors

Further provided herein are vectors comprising the nucleotide sequences encoding a multispecific or multifunctional molecule described herein. In one embodiment, the vectors comprise nucleotides encoding a multispecific or multifunctional molecule described herein. In one embodiment, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity. Methods and conditions for culturing the resulting transfected cells and for recovering the antibody molecule produced are known to those skilled in the art, and may be varied or optimized depending upon the specific expression vector and mammalian host cell employed, based upon the present description.

### Cells

In another aspect, the application features host cells and vectors containing the nucleic acids described herein. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell.

The invention also provides host cells comprising a nucleic acid encoding an antibody molecule as described herein.

In one embodiment, the host cells are genetically engineered to comprise nucleic acids encoding the antibody molecule.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The invention also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### Uses and Combination Therapies

Methods described herein include treating a cancer in a subject by using a multispecific molecule described herein, e.g., using a pharmaceutical composition described herein. Also provided are methods for reducing or ameliorating a symptom of a cancer in a subject, as well as methods for inhibiting the growth of a cancer and/or killing one or more cancer cells. In embodiments, the methods described herein decrease the size of a tumor and/or decrease the number of cancer cells in a subject administered with a described herein or a pharmaceutical composition described herein.

In embodiments, the cancer is a hematological cancer. In embodiments, the hematological cancer is a leukemia or a lymphoma. As used herein, a “hematologic cancer” refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AmoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

In embodiments, the cancer is a solid cancer. Exemplary solid cancers include, but are not limited to, ovarian cancer, rectal cancer, stomach cancer, testicular cancer, cancer of the anal region, uterine cancer, colon cancer, rectal cancer, renal-cell carcinoma, liver cancer, non-small cell carcinoma of the lung, cancer of the small intestine, cancer of the esophagus, melanoma, Kaposi's sarcoma, cancer of the endocrine system, cancer of the thyroid gland, cancer of the parathyroid gland, cancer of the adrenal gland, bone cancer, pancreatic cancer, skin cancer, cancer of the head or neck, cutaneous or intraocular malignant melanoma, uterine cancer, brain stem glioma, pituitary adenoma, epidermoid cancer, carcinoma of the cervix squamous cell cancer, carcinoma of the fallopian tubes, carcinoma of the endometrium, carcinoma of the vagina, sarcoma of soft tissue, cancer of the urethra, carcinoma of the vulva, cancer of the penis, cancer of the bladder, cancer of the kidney or ureter, carcinoma of the renal pelvis, spinal axis tumor, neoplasm of the central nervous system (CNS), primary CNS lymphoma, tumor angiogenesis, metastatic lesions of said cancers, or combinations thereof.

In certain embodiments, the cancer is an epithelial, mesenchymal or hematologic malignancy. In certain embodiments, the cancer treated is a solid tumor (e.g., carcinoid, carcinoma or sarcoma), a soft tissue tumor (e.g., a heme malignancy), and a metastatic lesion, e.g., a metastatic lesion of any of the cancers disclosed herein. In one embodiment, the cancer treated is a fibrotic or desmoplastic solid tumor, e.g., a tumor having one or more of: limited tumor perfusion, compressed blood vessels, fibrotic tumor interstitium, or increased interstitial fluid pressure. In one embodiment, the solid tumor is chosen from one or more of pancreatic (e.g., pancreatic adenocarcinoma or pancreatic ductal adenocarcinoma), breast, colon, colorectal, lung (e.g., small cell lung cancer (SCLC) or non-small cell lung cancer (NSCLC)), skin, ovarian, liver cancer, esophageal cancer, endometrial cancer, gastric cancer, head and neck cancer, kidney, or prostate cancer.

Examples of cancer include, but are not limited to, carcinoma, lymphoma, blastoma, sarcoma, and leukemia or lymphoid malignancies. More particular examples of such cancers are noted below and include: squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

In other embodiments, the multispecific molecule, as described above and herein, is used to treat a hyperproliferative disorder, e.g., a hyperproliferative connective tissue disorder (e.g., a hyperproliferative fibrotic disease). In one embodiment, the hyperproliferative fibrotic disease is multisystemic or organ-specific. Exemplary hyperproliferative fibrotic diseases include, but are not limited to, multisystemic (e.g., systemic sclerosis, multifocal fibrosclerosis, sclerodermatous graft-versus-host disease in bone marrow transplant recipients, nephrogenic systemic fibrosis, scleroderma), and organ-specific disorders (e.g., fibrosis of the eye, lung, liver, heart, kidney, pancreas, skin and other organs). In other embodiments, the disorder is chosen from liver cirrhosis or tuberculosis. In other embodiments, the disorder is leprosy.

In embodiments, the multispecific molecules (or pharmaceutical composition) are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the pharmaceutical composition (or multispecific molecules) to be administered can be determined by a physician with consideration of individual differences in tumor size, extent of infection or metastasis, age, weight, and condition of the subject. In embodiments, the pharmaceutical composition described herein can be administered at a dosage of 10⁴ to 10⁹ cells/kg body weight, e.g., 10⁵ to 10⁶ cells/kg body weight, including all integer values within those ranges. In embodiments, the pharmaceutical composition described herein can be administered multiple times at these dosages. In embodiments, the pharmaceutical composition described herein can be administered using infusion techniques described in immunotherapy (see, e.g., Rosenberg et al., New Eng. J. of Med. 319:1676, 1988).

In embodiments, the cancer is a myeloproliferative neoplasm, e.g., primary or idiopathic myelofibrosis (MF), essential thrombocytosis (ET), polycythemia vera (PV), or chronic myelogenous leukemia (CML). In embodiments, the cancer is myelofibrosis. In embodiments, the subject has myelofibrosis. In embodiments, the subject has a calreticulin mutation, e.g., a calreticulin mutation disclosed herein. In embodiments, the subject does not have the JAK2-V617F mutation. In embodiments, the subject has the JAK2-V617F mutation. In embodiments, the subject has a MPL mutation. In embodiments, the subject does not have a MPL mutation.

In embodiments, the cancer is a solid cancer. Exemplary solid cancers include, but are not limited to, ovarian cancer, rectal cancer, stomach cancer, testicular cancer, cancer of the anal region, uterine cancer, colon cancer, rectal cancer, renal-cell carcinoma, liver cancer, non-small cell carcinoma of the lung, cancer of the small intestine, cancer of the esophagus, melanoma, Kaposi's sarcoma, cancer of the endocrine system, cancer of the thyroid gland, cancer of the parathyroid gland, cancer of the adrenal gland, bone cancer, pancreatic cancer, skin cancer, cancer of the head or neck, cutaneous or intraocular malignant melanoma, uterine cancer, brain stem glioma, pituitary adenoma, epidermoid cancer, carcinoma of the cervix squamous cell cancer, carcinoma of the fallopian tubes, carcinoma of the endometrium, carcinoma of the vagina, sarcoma of soft tissue, cancer of the urethra, carcinoma of the vulva, cancer of the penis, cancer of the bladder, cancer of the kidney or ureter, carcinoma of the renal pelvis, spinal axis tumor, neoplasm of the central nervous system (CNS), primary CNS lymphoma, tumor angiogenesis, metastatic lesions of said cancers, or combinations thereof.

In embodiments, the multispecific molecules or pharmaceutical composition is administered to the subject parenterally. In embodiments, the cells are administered to the subject intravenously, subcutaneously, intratumorally, intranodally, intramuscularly, intradermally, or intraperitoneally. In embodiments, the cells are administered, e.g., injected, directly into a tumor or lymph node. In embodiments, the cells are administered as an infusion (e.g., as described in Rosenberg et al., New Eng. J. of Med. 319:1676, 1988) or an intravenous push. In embodiments, the cells are administered as an injectable depot formulation.

In embodiments, the subject is a mammal. In embodiments, the subject is a human, monkey, pig, dog, cat, cow, sheep, goat, rabbit, rat, or mouse. In embodiments, the subject is a human. In embodiments, the subject is a pediatric subject, e.g., less than 18 years of age, e.g., less than 17, 16, 15, 14, 13, 12, 11, 10, 9, 8, 7, 6, 5, 4, 3, 2, 1 or less years of age. In embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

### Combination Therapies

The multispecific or multifunctional molecules disclosed herein can be used in combination with a second therapeutic agent or procedure.

In embodiments, the multispecific or multifunctional molecule and the second therapeutic agent or procedure are administered/performed after a subject has been diagnosed with a cancer, e.g., before the cancer has been eliminated from the subject. In embodiments, the multispecific or multifunctional molecule and the second therapeutic agent or procedure are administered/performed simultaneously or concurrently. For example, the delivery of one treatment is still occurring when the delivery of the second commences, e.g., there is an overlap in administration of the treatments. In other embodiments, the multispecific or multifunctional molecule and the second therapeutic agent or procedure are administered/performed sequentially. For example, the delivery of one treatment ceases before the delivery of the other treatment begins.

In embodiments, combination therapy can lead to more effective treatment than monotherapy with either agent alone. In embodiments, the combination of the first and second treatment is more effective (e.g., leads to a greater reduction in symptoms and/or cancer cells) than the first or second treatment alone. In embodiments, the combination therapy permits use of a lower dose of the first or the second treatment compared to the dose of the first or second treatment normally required to achieve similar effects when administered as a monotherapy. In embodiments, the combination therapy has a partially additive effect, wholly additive effect, or greater than additive effect.

In one embodiment, the multispecific or multifunctional molecule is administered in combination with a therapy, e.g., a cancer therapy (e.g., one or more of anti-cancer agents, immunotherapy, photodynamic therapy (PDT), surgery and/or radiation). The terms “chemotherapeutic,” “chemotherapeutic agent,” and “anti-cancer agent” are used interchangeably herein. The administration of the multispecific or multifunctional molecule and the therapy, e.g., the cancer therapy, can be sequential (with or without overlap) or simultaneous. Administration of the multispecific or multifunctional molecule can be continuous or intermittent during the course of therapy (e.g., cancer therapy). Certain therapies described herein can be used to treat cancers and non-cancerous diseases. For example, PDT efficacy can be enhanced in cancerous and non-cancerous conditions (e.g., tuberculosis) using the methods and compositions described herein (reviewed in, e.g., Agostinis, P. et al. (2011) CA Cancer J. Clin. 61:250-281).

### Anti-Cancer Therapies

In other embodiments, the multispecific or multifunctional molecule is administered in combination with a low or small molecular weight chemotherapeutic agent. Exemplary low or small molecular weight chemotherapeutic agents include, but not limited to, 13-cis-retinoic acid (isotretinoin, ACCUTANE®), 2-CdA (2-chlorodeoxyadenosine, cladribine, LEUSTATIN™), 5-azacitidine (azacitidine, VIDAZA®), 5-fluorouracil (5-FU, fluorouracil, ADRUCIL®), 6-mercaptopurine (6-MP, mercaptopurine, PURINETHOL®), 6-TG (6-thioguanine, thioguanine, THIOGUANINE TABLOID®), abraxane (paclitaxel protein-bound), actinomycin-D (dactinomycin, COSMEGEN®), alitretinoin (PANRETIN®), all-transretinoic acid (ATRA, tretinoin, VESANOID®), altretamine (hexamethylmelamine, HMM, HEXALEN®), amethopterin (methotrexate, methotrexate sodium, MTX, TREXALL™, RHEUMATREX®), amifostine (ETHYOL®), arabinosylcytosine (Ara-C, cytarabine, CYTOSAR-U®), arsenic trioxide (TRISENOX®), asparaginase (Erwinia L-asparaginase, L-asparaginase, ELSPAR®, KIDROLASE®), BCNU (carmustine, BiCNU®), bendamustine (TREANDA®), bexarotene (TARGRETIN®), bleomycin (BLENOXANE®), busulfan (BUSULFEX®, MYLERAN®), calcium leucovorin (Citrovorum Factor, folinic acid, leucovorin), camptothecin-11 (CPT-11, irinotecan, CAMPTOSAR®), capecitabine (XELODA®), carboplatin (PARAPLATIN®), carmustine wafer (prolifeprospan 20 with carmustine implant, GLIADEL® wafer), CCI-779 (temsirolimus, TORISEL®), CCNU (lomustine, CeeNU), CDDP (cisplatin, PLATINOL®, PLATINOL-AQ®), chlorambucil (leukeran), cyclophosphamide (CYTOXAN®, NEOSAR®), dacarbazine (DIC, DTIC, imidazole carboxamide, DTIC-DOME®), daunomycin (daunorubicin, daunorubicin hydrochloride, rubidomycin hydrochloride, CERUBIDINE®), decitabine (DACOGEN®), dexrazoxane (ZINECARD®), DHAD (mitoxantrone, NOVANTRONE®), docetaxel (TAXOTERE®), doxorubicin (ADRIAMYCIN®, RUBEX®), epirubicin (ELLENCE™), estramustine (EMCYT®), etoposide (VP-16, etoposide phosphate, TOPOSAR®, VEPESID®, ETOPOPHOS®), floxuridine (FUDR®), fludarabine (FLUDARA®), fluorouracil (cream) (CARAC™, EFUDEX®, FLUOROPLEX®), gemcitabine (GEMZAR®), hydroxyurea (HYDREA®, DROXIA™, MYLOCEL™), idarubicin (IDAMYCIN®), ifosfamide (IFEX®), ixabepilone (IXEMPRA™), LCR (leurocristine, vincristine, VCR, ONCOVIN®, VINCASAR PFS®), L-PAM (L-sarcolysin, melphalan, phenylalanine mustard, ALKERAN®), mechlorethamine (mechlorethamine hydrochloride, mustine, nitrogen mustard, MUSTARGEN®), mesna (MESNEX™), mitomycin (mitomycin-C, MTC, MUTAMYCIN®), nelarabine (ARRANON®), oxaliplatin (ELOXATIN™), paclitaxel (TAXOL®, ONXAL™), pegaspargase (PEG-L-asparaginase, ONCOSPAR®), PEMETREXED (ALIMTA®), pentostatin (NIPENT®), procarbazine (MATULANE®), streptozocin (ZANOSAR®), temozolomide (TEMODAR®), teniposide (VM-26, VUMON®), TESPA (thiophosphoamide, thiotepa, TSPA, THIOPLEX®), topotecan (HYCAMTIN®), vinblastine (vinblastine sulfate, vincaleukoblastine, VLB, ALKABAN-AQ®, VELBAN®), vinorelbine (vinorelbine tartrate, NAVELBINE®), and vorinostat (ZOLINZA®).

In another embodiment, the multispecific or multifunctional molecule is administered in conjunction with a biologic. Biologics useful in the treatment of cancers are known in the art and a binding molecule of the invention may be administered, for example, in conjunction with such known biologics. For example, the FDA has approved the following biologics for the treatment of breast cancer: HERCEPTIN® (trastuzumab, Genentech Inc., South San Francisco, Calif.; a humanized monoclonal antibody that has anti-tumor activity in HER2-positive breast cancer); FASLODEX® (fulvestrant, AstraZeneca Pharmaceuticals, LP, Wilmington, Del.; an estrogen-receptor antagonist used to treat breast cancer); ARIMIDEX® (anastrozole, AstraZeneca Pharmaceuticals, LP; a nonsteroidal aromatase inhibitor which blocks aromatase, an enzyme needed to make estrogen); Aromasin® (exemestane, Pfizer Inc., New York, N.Y.; an irreversible, steroidal aromatase inactivator used in the treatment of breast cancer); FEMARA® (letrozole, Novartis Pharmaceuticals, East Hanover, N.J.; a nonsteroidal aromatase inhibitor approved by the FDA to treat breast cancer); and NOLVADEX® (tamoxifen, AstraZeneca Pharmaceuticals, LP; a nonsteroidal antiestrogen approved by the FDA to treat breast cancer). Other biologics with which the binding molecules of the invention may be combined include: AVASTIN® (bevacizumab, Genentech Inc.; the first FDA-approved therapy designed to inhibit angiogenesis); and ZEVALIN® (ibritumomab tiuxetan, Biogen Idec, Cambridge, Mass.; a radiolabeled monoclonal antibody currently approved for the treatment of B-cell lymphomas).

In addition, the FDA has approved the following biologics for the treatment of colorectal cancer: AVASTIN®; ERBITUX® (cetuximab, ImClone Systems Inc., New York, N.Y., and Bristol-Myers Squibb, New York, N.Y.; is a monoclonal antibody directed against the epidermal growth factor receptor (EGFR)); GLEEVEC® (imatinib mesylate; a protein kinase inhibitor); and ERGAMISOL® (levamisole hydrochloride, Janssen Pharmaceutica Products, LP, Titusville, N.J.; an immunomodulator approved by the FDA in 1990 as an adjuvant treatment in combination with 5-fluorouracil after surgical resection in patients with Dukes' Stage C colon cancer).

For the treatment of lung cancer, exemplary biologics include TARCEVA® (erlotinib HCL, OSI Pharmaceuticals Inc., Melville, N.Y.; a small molecule designed to target the human epidermal growth factor receptor 1 (HER1) pathway).

For the treatment of multiple myeloma, exemplary biologics include VELCADE® Velcade (bortezomib, Millennium Pharmaceuticals, Cambridge Mass.; a proteasome inhibitor). Additional biologics include THALIDOMID® (thalidomide, Clegene Corporation, Warren, N.J.; an immunomodulatory agent and appears to have multiple actions, including the ability to inhibit the growth and survival of myeloma cells and anti-angiogenesis).

Additional exemplary cancer therapeutic antibodies include, but are not limited to, 3F8, abagovomab, adecatumumab, afutuzumab, alacizumab pegol, alemtuzumab (CAMPATH®, MABCAMPATH®), altumomab pentetate (HYBRI-CEAKER®), anatumomab mafenatox, anrukinzumab (IMA-638), apolizumab, arcitumomab (CEA-SCAN®), bavituximab, bectumomab (LYMPHOSCAN®), belimumab (BENLYSTA®, LYMPHOSTAT-B®), besilesomab (SCINTIMUN®), bevacizumab (AVASTIN®), bivatuzumab mertansine, blinatumomab, brentuximab vedotin, cantuzumab mertansine, capromab pendetide (PROSTASCINTT), catumaxomab (REMOVAB®), CC49, cetuximab (C225, ERBITUX®), citatuzumab bogatox, cixutumumab, clivatuzumab tetraxetan, conatumumab, dacetuzumab, denosumab (PROLIA®), detumomab, ecromeximab, edrecolomab (PANOREX®), elotuzumab, epitumomab cituxetan, epratuzumab, ertumaxomab (REXOMUN®), etaracizumab, farletuzumab, figitumumab, fresolimumab, galiximab, gemtuzumab ozogamicin (MYLOTARG®), girentuximab, glembatumumab vedotin, ibritumomab (ibritumomab tiuxetan, ZEVALIN®), igovomab (INDIMACIS-125®), intetumumab, inotuzumab ozogamicin, ipilimumab, iratumumab, labetuzumab (CEA-CIDE®), lexatumumab, lintuzumab, lucatumumab, lumiliximab, mapatumumab, matuzumab, milatuzumab, minretumomab, mitumomab, nacolomab tafenatox, naptumomab estafenatox, necitumumab, nimotuzumab (THERACIM®, THERALOC®), nofetumomab merpentan (VERLUMA®), ofatumumab (ARZERRA®), olaratumab, oportuzumab monatox, oregovomab (OVAREX®), panitumumab (VECTIBIX®), pemtumomab (THERAGYN®), pertuzumab (OMNITARG®), pintumomab, pritumumab, ramucirumab, ranibizumab (LUCENTIS®), rilotumumab, rituximab (MABTHERA®, RITUXAN®), robatumumab, satumomab pendetide, sibrotuzumab, siltuximab, sontuzumab, tacatuzumab tetraxetan (AFP-CIDE®), taplitumomab paptox, tenatumomab, TGN1412, ticilimumab (tremelimumab), tigatuzumab, TNX-650, tositumomab (BEXXAR®), trastuzumab (HERCEPTIN®), tremelimumab, tucotuzumab celmoleukin, veltuzumab, volociximab, votumumab (HUMASPECT®), zalutumumab (HUMAX-EGFR®), and zanolimumab (HUMAX-CD4®).

In other embodiments, the multispecific or multifunctional molecule is administered in combination with a viral cancer therapeutic agent. Exemplary viral cancer therapeutic agents include, but not limited to, vaccinia virus (vvDD-CDSR), carcinoembryonic antigen-expressing measles virus, recombinant vaccinia virus (TK-deletion plus GM-CSF), Seneca Valley virus-001, Newcastle virus, coxsackie virus A21, GL-ONC1, EBNA1 C-terminal/LMP2 chimeric protein-expressing recombinant modified vaccinia Ankara vaccine, carcinoembryonic antigen-expressing measles virus, G207 oncolytic virus, modified vaccinia virus Ankara vaccine expressing p53, OncoVEX GM-CSF modified herpes-simplex 1 virus, fowlpox virus vaccine vector, recombinant vaccinia prostate-specific antigen vaccine, human papillomavirus 16/18 L1 virus-like particle/AS04 vaccine, MVA-EBNA1/LMP2 Inj. Vaccine, quadrivalent HPV vaccine, quadrivalent human papillomavirus (types 6, 11, 16, 18) recombinant vaccine (GARDASIL®), recombinant fowlpox-CEA(6D)/TRICOM vaccine; recombinant vaccinia-CEA(6D)-TRICOM vaccine, recombinant modified vaccinia Ankara-5T4 vaccine, recombinant fowlpox-TRICOM vaccine, oncolytic herpes virus NV1020, HPV L1 VLP vaccine V504, human papillomavirus bivalent (types 16 and 18) vaccine (CERVARIX®), herpes simplex virus HF10, Ad5CMV-p53 gene, recombinant vaccinia DF3/MUC1 vaccine, recombinant vaccinia-MUC-1 vaccine, recombinant vaccinia-TRICOM vaccine, ALVAC MART-1 vaccine, replication-defective herpes simplex virus type I (HSV-1) vector expressing human Preproenkephalin (NP2), wild-type reovirus, reovirus type 3 Dearing (REOLYSIN®), oncolytic virus HSV1716, recombinant modified vaccinia Ankara (MVA)-based vaccine encoding Epstein-Barr virus target antigens, recombinant fowlpox-prostate specific antigen vaccine, recombinant vaccinia prostate-specific antigen vaccine, recombinant vaccinia-B7.1 vaccine, rAd-p53 gene, Ad5-delta24RGD, HPV vaccine 580299, JX-594 (thymidine kinase-deleted vaccinia virus plus GM-CSF), HPV-16/18 L1/AS04, fowlpox virus vaccine vector, vaccinia-tyrosinase vaccine, MEDI-517 HPV-16/18 VLP AS04 vaccine, adenoviral vector containing the thymidine kinase of herpes simplex virus TK99UN, HspE7, FP253/Fludarabine, ALVAC(2) melanoma multi-antigen therapeutic vaccine, ALVAC-hB7.1, canarypox-hIL-12 melanoma vaccine, Ad-REIC/Dkk-3, rAd-IFN SCH 721015, TIL-Ad-INFg, Ad-ISF35, and coxsackievirus A21 (CVA21, CAVATAK®).

In other embodiments, the multispecific or multifunctional molecule is administered in combination with a nanopharmaceutical. Exemplary cancer nanopharmaceuticals include, but not limited to, ABRAXANE® (paclitaxel bound albumin nanoparticles), CRLX101 (CPT conjugated to a linear cyclodextrin-based polymer), CRLX288 (conjugating docetaxel to the biodegradable polymer poly (lactic-co-glycolic acid)), cytarabine liposomal (liposomal Ara-C, DEPOCYT™), daunorubicin liposomal (DAUNOXOME®), doxorubicin liposomal (DOXIL®, CAELYX®), encapsulated-daunorubicin citrate liposome (DAUNOXOME®), and PEG anti-VEGF aptamer (MACUGEN®).

In some embodiments, the multispecific or multifunctional molecule is administered in combination with paclitaxel or a paclitaxel formulation, e.g., TAXOL®, protein-bound paclitaxel (e.g., ABRAXANE®). Exemplary paclitaxel formulations include, but are not limited to, nanoparticle albumin-bound paclitaxel (ABRAXANE®, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., Bioorganic & Medicinal Chemistry Letters (2007) 17:617-620).

Exemplary RNAi and antisense RNA agents for treating cancer include, but not limited to, CALAA-01, siG12D LODER (Local Drug EluteR), and ALN-VSP02.

Other cancer therapeutic agents include, but not limited to, cytokines (e.g., aldesleukin (IL-2, Interleukin-2, PROLEUKIN®), alpha Interferon (IFN-alpha, Interferon alfa, INTRON® A (Interferon alfa-2b), ROFERON-A® (Interferon alfa-2a)), Epoetin alfa (PROCRIT®), filgrastim (G-CSF, Granulocyte—Colony Stimulating Factor, NEUPOGEN®), GM-CSF (Granulocyte Macrophage Colony Stimulating Factor, sargramostim, LEUKINE™), IL-II (Interleukin-11, oprelvekin, NEUMEGA®), Interferon alfa-2b (PEG conjugate) (PEG interferon, PEG-INTRON™), and pegfilgrastim (NEULASTA™)), hormone therapy agents (e.g., aminoglutethimide (CYTADREN®), anastrozole (ARIMIDEX®), bicalutamide (CASODEX®), exemestane (AROMASIN®), fluoxymesterone (HALOTESTIN®), flutamide (EULEXIN®), fulvestrant (FASLODEX®), goserelin (ZOLADEX®), letrozole (FEMARA®), leuprolide (ELIGARD™, LUPRON®, LUPRON DEPOT®, VIADUR™), megestrol (megestrol acetate, MEGACE®), nilutamide (ANANDRON®, NILANDRON®), octreotide (octreotide acetate, SANDOSTATIN®, SANDOSTATIN LAR®), raloxifene (EVISTA®), romiplostim (NPLATE®), tamoxifen (NOVALDEX®), and toremifene (FARESTON®)), phospholipase A2 inhibitors (e.g., anagrelide (AGRYLIN®)), biologic response modifiers (e.g., BCG (THERACYS®, TICE®), and Darbepoetin alfa (ARANESP®)), target therapy agents (e.g., bortezomib (VELCADE®), dasatinib (SPRYCEL™), denileukin diftitox (ONTAK®), erlotinib (TARCEVA®), everolimus (AFINITOR®), gefitinib (IRESSA®), imatinib mesylate (STI-571, GLEEVEC™), lapatinib (TYKERB®), sorafenib (NEXAVAR®), and SU11248 (sunitinib, SUTENT®)), immunomodulatory and antiangiogenic agents (e.g., CC-5013 (lenalidomide, REVLIMID®), and thalidomide (THALOMID®)), glucocorticosteroids (e.g., cortisone (hydrocortisone, hydrocortisone sodium phosphate, hydrocortisone sodium succinate, ALA-CORT®, HYDROCORT ACETATE®, hydrocortone phosphate LANACORT®, SOLU-CORTEF®), decadron (dexamethasone, dexamethasone acetate, dexamethasone sodium phosphate, DEXASONE®, DIODEX®, HEXADROL®, MAXIDEX®), methylprednisolone (6-methylprednisolone, methylprednisolone acetate, methylprednisolone sodium succinate, DURALONE®, MEDRALONE®, MEDROL®, M-PREDNISOL®, SOLU-MEDROL®), prednisolone (DELTA-CORTEF®, ORAPRED®, PEDIAPRED®, PRELONE®), and prednisone (DELTASONE®, LIQUID PRED®, METICORTEN®, ORASONE®)), and bisphosphonates (e.g., pamidronate (AREDIA®), and zoledronic acid (ZOMETA®))

In some embodiments, the multispecific or multifunctional molecule is used in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., an antibody against VEGF, a VEGF trap, a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-ß inhibitor)), a RAF-1 inhibitor, a KIT inhibitor and a RET inhibitor. In some embodiments, the anti-cancer agent used in combination with the AHCM agent is selected from the group consisting of: axitinib (AGO13736), bosutinib (SKI-606), cediranib (RECENTIN™ AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951(tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010), MGCD-265, motesanib diphosphate (AMG-706), MP-470, OSI-930, Pazopanib Hydrochloride, PD173074, Sorafenib Tosylate (Bay 43-9006), SU 5402, TSU-68(SU6668), vatalanib, XL880 (GSK1363089, EXEL-2880). Selected tyrosine kinase inhibitors are chosen from sunitinib, erlotinib, gefitinib, or sorafenib. In one embodiment, the tyrosine kinase inhibitor is sunitinib.

In one embodiment, the multispecific or multifunctional molecule is administered in combination with one of more of: an anti-angiogenic agent, or a vascular targeting agent or a vascular disrupting agent. Exemplary anti-angiogenic agents include, but are not limited to, VEGF inhibitors (e.g., anti-VEGF antibodies (e.g., bevacizumab); VEGF receptor inhibitors (e.g., itraconazole); inhibitors of cell proliferatin and/or migration of endothelial cells (e.g., carboxyamidotriazole, TNP-470); inhibitors of angiogenesis stimulators (e.g., suramin), among others. A vascular-targeting agent (VTA) or vascular disrupting agent (VDA) is designed to damage the vasculature (blood vessels) of cancer tumors causing central necrosis (reviewed in, e.g., Thorpe, P. E. (2004) Clin. Cancer Res. Vol. 10:415-427). VTAs can be small-molecule. Exemplary small-molecule VTAs include, but are not limited to, microtubule destabilizing drugs (e.g., combretastatin A-4 disodium phosphate (CA4P), ZD6126, AVE8062, Oxi 4503); and vadimezan (ASA404).

### Immune Checkpoint Inhibitors

In other embodiments, methods described herein comprise use of an immune checkpoint inhibitor in combination with the agent, or the multispecific or multifunctional molecule as described herein. The methods can be used in a therapeutic protocol in vivo.

In embodiments, an immune checkpoint inhibitor inhibits a checkpoint molecule. Exemplary checkpoint molecules include but are not limited to CTLA4, PD1, PD-L1, PD-L2, TIM3, LAG3, CD160, 2B4, CD80, CD86, B7-H3 (CD276), B7-H4 (VTCN1), HVEM (TNFRSFi4 or CD270), BTLA, KIR, MHC class I, MHC class II, GAL9, VISTA, BTLA, TIGIT, LAIRI, and A2aR. See, e.g., Pardoll. Nat. Rev. Cancer 12.4(2012):252-64, incorporated herein by reference.

In embodiments, the immune checkpoint inhibitor is a PD-1 inhibitor, e.g., an anti-PD-1 antibody such as Nivolumab, Pembrolizumab or Pidilizumab. Nivolumab (also called MDX-1106, MDX-1106-04, ONO-4538, or BMS-936558) is a fully human IgG4 monoclonal antibody that specifically inhibits PD1. See, e.g., U.S. Pat. No. 8,008,449 and WO2006/121168. Pembrolizumab (also called Lambrolizumab, MK-3475, MK03475, SCH-900475 or KEYTRUDA®; Merck) is a humanized IgG4 monoclonal antibody that binds to PD-1. See, e.g., Hamid, O. et al. (2013) New England Journal of Medicine 369 (2): 134-44, U.S. Pat. No. 8,354,509 and WO2009/114335. Pidilizumab (also called CT-011 or Cure Tech) is a humanized IgG1k monoclonal antibody that binds to PD1. See, e.g., WO2009/101611. In one embodiment, the inhibitor of PD-1 is an antibody molecule having a sequence substantially identical or similar thereto, e.g., a sequence at least 85%, 90%, 95% identical or higher to the sequence of Nivolumab, Pembrolizumab or Pidilizumab. Additional anti-PD1 antibodies, e.g., AMP 514 (Amplimmune), are described, e.g., in U.S. Pat. No. 8,609,089, US 2010028330, and/or US 20120114649.

In some embodiments, the PD-1 inhibitor is an immunoadhesin, e.g., an immunoadhesin comprising an extracellular/PD-1 binding portion of a PD-1 ligand (e.g., PD-L1 or PD-L2) that is fused to a constant region (e.g., an Fc region of an immunoglobulin). In embodiments, the PD-1 inhibitor is AMP-224 (B7-DCIg, e.g., described in WO2011/066342 and WO2010/027827), a PD-L2 Fc fusion soluble receptor that blocks the interaction between B7-H1 and PD-1.

In embodiments, the immune checkpoint inhibitor is a PD-L1 inhibitor, e.g., an antibody molecule. In some embodiments, the PD-L1 inhibitor is YW243.55.570, MPDL3280A, MEDI-4736, MSB-0010718C, or MDX-1105. In some embodiments, the anti-PD-L1 antibody is MSB0010718C (also called A09-246-2; Merck Serono), which is a monoclonal antibody that binds to PD-L1. Exemplary humanized anti-PD-L1 antibodies are described, e.g., in WO2013/079174. In one embodiment, the PD-L1 inhibitor is an anti-PD-L1 antibody, e.g., YW243.55.570. The YW243.55.570 antibody is described, e.g., in WO 2010/077634. In one embodiment, the PD-L1 inhibitor is MDX-1105 (also called BMS-936559), which is described, e.g., in WO2007/005874. In one embodiment, the PD-L1 inhibitor is MDPL3280A (Genentech/Roche), which is a human Fc-optimized IgG1 monoclonal antibody against PD-L1. See, e.g., U.S. Pat. No. 7,943,743 and U.S Publication No.: 20120039906. In one embodiment, the inhibitor of PD-L1 is an antibody molecule having a sequence substantially identical or similar thereto, e.g., a sequence at least 85%, 90%, 95% identical or higher to the sequence of YW243.55.570, MPDL3280A, MEDI-4736, MSB-0010718C, or MDX-1105.

In embodiments, the immune checkpoint inhibitor is a PD-L2 inhibitor, e.g., AMP-224 (which is a PD-L2 Fc fusion soluble receptor that blocks the interaction between PD1 and B7-H1. See, e.g., WO2010/027827 and WO2011/066342.

In one embodiment, the immune checkpoint inhibitor is a LAG-3 inhibitor, e.g., an anti-LAG-3 antibody molecule. In embodiments, the anti-LAG-3 antibody is BMS-986016 (also called BMS986016; Bristol-Myers Squibb). BMS-986016 and other humanized anti-LAG-3 antibodies are described, e.g., in US 2011/0150892, WO2010/019570, and WO2014/008218.

In embodiments, the immune checkpoint inhibitor is a TIM-3 inhibitor, e.g., anti-TIM3 antibody molecule, e.g., described in U.S. Pat. No. 8,552,156, WO 2011/155607, EP 2581113 and U.S Publication No.: 2014/044728.

In embodiments, the immune checkpoint inhibitor is a CTLA-4 inhibitor, e.g., anti-CTLA-4 antibody molecule. Exemplary anti-CTLA4 antibodies include Tremelimumab (IgG2 monoclonal antibody from Pfizer, formerly known as ticilimumab, CP-675,206); and Ipilimumab (also called MDX-010, CAS No. 477202-00-9). Other exemplary anti-CTLA-4 antibodies are described, e.g., in U.S. Pat. No. 5,811,097.

### Exemplary Embodiments of Agents

In an aspect, provided herein is, inter alia, a composition comprising an agent that comprises an antigen binding domain that binds to NKp30, wherein the antigen binding domain comprises a heavy chain variable region (VH) comprising:
(i) a heavy chain complementarity determining region 3 (VHCDR3) comprising the sequence of GDWHYFDY (SEQ ID NO: 504); and
(ii) a heavy chain complementarity determining region 1 (VHCDR1) comprising the sequence of GFSIIRVGYHWN (SEQ ID NO: 502), a heavy chain complementarity determining region 2 (VHCDR2) comprising the sequence of YIHRSGSTSYNPSLKS (SEQ ID NO: 503), or a combination thereof.

In an aspect, provided herein is, inter alia, a composition comprising an agent that comprises an antigen binding domain that binds to NKp30, wherein the antigen binding domain comprises a heavy chain variable region (VH) comprising:
(i) a heavy chain complementarity determining region 3 (VHCDR3) comprising the sequence of GDWHYFDY (SEQ ID NO: 504) or a sequence thereof with no more than 1, 2, 3, or 4 mutations, such as substitutions, additions, or deletions; and
(ii) a heavy chain complementarity determining region 1 (VHCDR1) comprising the sequence of GFSIIRVGYHWN (SEQ ID NO: 502) or a sequence thereof with no more than 1, 2, 3, or 4 mutations, such as substitutions, additions, or deletions, a heavy chain complementarity determining region 2 (VHCDR2) comprising the sequence of YIHRSGSTSYNPSLKS (SEQ ID NO: 503) or a sequence thereof with no more than 1, 2, 3, or 4 mutations, such as substitutions, additions, or deletions, or any combination thereof.

In some embodiments, the antigen binding domain comprises a VH comprising the VHCDR1 comprising the sequence of GFSIIRVGYHWN (SEQ ID NO: 502), the VHCDR2 comprising the sequence of YIHRSGSTSYNPSLKS (SEQ ID NO: 503), and the VHCDR3 comprising the sequence of GDWHYFDY (SEQ ID NO: 504).

In some embodiments, the antigen binding domain comprises a VH comprising the VHCDR1 comprising the sequence of GFSIIRVGYHWN (SEQ ID NO: 502) or a sequence thereof with no more than 1, 2, 3, or 4 mutations, such as substitutions, additions, or deletions, the VHCDR2 comprising the sequence of YIHRSGSTSYNPSLKS (SEQ ID NO: 503) or a sequence thereof with no more than 1, 2, 3, or 4 mutations, such as substitutions, additions, or deletions, and the VHCDR3 comprising the sequence of GDWHYFDY (SEQ ID NO: 504) or a sequence thereof with no more than 1, 2, 3, or 4 mutations, such as substitutions, additions, or deletions.

In some embodiments, the antigen binding domain comprises a light chain variable region (VL) comprising a light chain complementarity determining region 1 (VLCDR1) comprising the sequence of SGEKLSDKYVH (SEQ ID NO: 26), a light chain complementarity determining region 2 (VLCDR2) comprising the sequence of ENDRRPS (SEQ ID NO: 28), a light chain complementarity determining region 3 (VLCDR3) comprising the sequences of QFWDSTNSAV (SEQ ID NO: 86), or any combination thereof.

In some embodiments, the antigen binding domain comprises a light chain variable region (VL) comprising a light chain complementarity determining region 1 (VLCDR1) comprising the sequence of SGEKLSDKYVH (SEQ ID NO: 26) or a sequence thereof with no more than 1, 2, 3, or 4 mutations, such as substitutions, additions, or deletions, a light chain complementarity determining region 2 (VLCDR2) comprising the sequence of ENDRRPS (SEQ ID NO: 28) or a sequence thereof with no more than 1, 2, 3, or 4 mutations, such as substitutions, additions, or deletions, a light chain complementarity determining region 3 (VLCDR3) comprising the sequences of QFWDSTNSAV (SEQ ID NO: 86) or a sequence thereof with no more than 1, 2, 3, or 4 mutations, such as substitutions, additions, or deletions, or any combination thereof.

In some embodiments, the antigen binding domain comprises a VL comprising a VLCDR1 comprising the sequence of SGEKLSDKYVH (SEQ ID NO: 26), a VLCDR2 comprising the sequence of ENDRRPS (SEQ ID NO: 28), and a VLCDR3 comprising the sequences of QFWDSTNSAV (SEQ ID NO: 86).

In some embodiments, the antigen binding domain comprises a VL comprising a VLCDR1 comprising the sequence of SGEKLSDKYVH (SEQ ID NO: 26) or a sequence thereof with no more than 1, 2, 3, or 4 mutations, such as substitutions, additions, or deletions, a VLCDR2 comprising the sequence of ENDRRPS (SEQ ID NO: 28) or a sequence thereof with no more than 1, 2, 3, or 4 mutations, such as substitutions, additions, or deletions, and a VLCDR3 comprising the sequences of QFWDSTNSAV (SEQ ID NO: 86) or a sequence thereof with no more than 1, 2, 3, or 4 mutations, such as substitutions, additions, or deletions.

In some embodiments, the antigen binding domain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 505.

In some embodiments, the antigen binding domain comprises a VH comprising the sequence of SEQ ID NO: 505.

In some embodiments, the antigen binding domain comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 13.

In some embodiments, the antigen binding domain comprises a VL comprising the sequence of SEQ ID NO: 13.

In some embodiments, the antigen binding domain comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 505 and a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 13.

In some embodiments, the antigen binding domain comprises a VH comprising the sequence of SEQ ID NO: 505 and a VL comprising the sequence of SEQ ID NO: 13.

In some embodiments, the antigen binding domain is a full antibody or antigen-binding fragment thereof, a bivalent antibody, bispecific antibody, or a biparatopic antibody.

In some embodiments, the antigen binding domain is a Fab, F(ab′)2, Fv, an single chain Fv (scFv), a diabody, a single domain antibody, or a camelid antibody.

In some embodiments, the antigen binding domain is a Fab or an scFv.

In some embodiments, the antigen binding domain comprises one or more heavy chain constant regions selected from the group consisting of IgG1 heavy chain constant region or functional fragment thereof, IgG2 heavy chain constant region or functional fragment thereof, IgG3 heavy chain constant region or functional fragment thereof, IgGA1 heavy chain constant region or functional fragment thereof, IgGA2 heavy chain constant region or functional fragment thereof, IgG4 heavy chain constant region or functional fragment thereof, IgJ heavy chain constant region or functional fragment thereof, IgM heavy chain constant region or functional fragment thereof, IgD heavy chain constant region or functional fragment thereof, and IgE heavy chain constant region or functional fragment thereof.

In some embodiments, the one or more heavy chain constant regions comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one heavy chain constant region sequences listed in Tables 3, 7, 8, 16, 17, 39, 40, 42C, 43A, 43B, and 46-51.

In some embodiments, the one or more heavy chain constant regions comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 73, the sequence of SEQ ID NO: 76, the sequence of SEQ ID NO: 74, the sequence of SEQ ID NO: 75, the sequence of SEQ ID NO: 76, the sequence of SEQ ID NO: 40, the sequence of SEQ ID NO: 41, the sequence of SEQ ID NO: 42, the sequence of SEQ ID NO: 3645, the sequence of SEQ ID NO: 3646, the sequence of SEQ ID NO: 3649, the sequence of SEQ ID NO: 3451, the sequence of SEQ ID NO: 506, the sequence of SEQ ID NO: 507, the sequence of SEQ ID NO: 3647, the sequence of SEQ ID NO: 3648, the sequence of SEQ ID NO: 508 or 8686, the sequence of SEQ ID NO: 513, the sequence of SEQ ID NO: 514, the sequence of SEQ ID NO: 515, the sequence of SEQ ID NO: 516, the sequence of SEQ ID NO: 3447, or the sequence of SEQ ID NO: 511.

In some embodiments, the antigen binding domain comprises a kappa light chain constant region or functional fragment thereof, a lambda light chain constant region or functional fragment thereof, or a combination thereof.

In some embodiments, the kappa light chain constant region comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one light chain constant region sequences listed in Tables 3, 7, 8, 16, 17, 39, 40, 42C, 43A, 43B, and 46-51.

In some embodiments, the kappa light chain constant region comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 39 or the sequence of SEQ ID NO: 3644.

In some embodiments, the agent is a polypeptide molecule.

In some embodiments, the agent is a multifunctional molecule.

In some embodiments, the agent is a multispecific molecule.

In some embodiments, the agent comprises at least one cytokine molecule or a functional fragment or variant thereof or a functional fragment or variant thereof, an antigen binding domain that binds to T cell receptor beta chain constant domain (TRBC), a second antigen binding domain that binds to NKp30, an immune cell engager, a tumor-targeting moiety, or any combination thereof.

In some embodiments, the agent comprises at least two non-contiguous polypeptide chains comprising a first polypeptide chain and a second polypeptide chain; wherein the first polypeptide chain comprises, from N-terminus to C-terminus, the following configuration: A-[a first member of a dimerization module]-C, And the second polypeptide chain comprises, from N-terminus to C-terminus, the following configuration: B-[a second member of a dimerization module]-D; wherein the first member of the dimerization module comprises a first immunoglobulin constant domain; and the second member of the dimerization module comprises a second immunoglobulin constant domain; wherein at least one of A, B, C, and D is the antigen binding domain; and wherein the first polypeptide chain and the second polypeptide chain dimerize via interaction between the first member of the dimerization module and the second member of the dimerization module.

In some embodiments, (i) A is the antigen binding domain, and B, C, and D are independently (a) absent, (b) at least one cytokine molecule or a functional fragment or variant thereof, (c) an antigen binding domain that binds to T cell receptor beta chain constant domain (TRBC), (d) a second antigen binding domain that binds to NKp30, (e) an immune cell engager, or (f) a tumor-targeting moiety; (ii) B is the antigen binding domain, and A, C, and D are independently (a) absent, (b) at least one cytokine molecule or a functional fragment or variant thereof, (c) an antigen binding domain that binds to T cell receptor beta chain constant domain (TRBC), (d) a second antigen binding domain that binds to NKp30, (e) an immune cell engager, or (f) a tumor-targeting moiety; (iii) C is the antigen binding domain, and A, B, and D are independently (a) absent, (b) at least one cytokine molecule or a functional fragment or variant thereof, (c) an antigen binding domain that binds to T cell receptor beta chain constant domain (TRBC), (d) a second antigen binding domain that binds to NKp30, (e) an immune cell engager, or (f) a tumor-targeting moiety; (iv) D is the antigen binding domain, and A, B, and C are independently (a) absent, (b) at least one cytokine molecule or a functional fragment or variant thereof, (c) an antigen binding domain that binds to T cell receptor beta chain constant domain (TRBC), (d) a second antigen binding domain that binds to NKp30, (e) an immune cell engager, or (f) a tumor-targeting moiety.

In some embodiments, the agent comprises at least one cytokine molecule or a functional fragment or variant thereof.

In some embodiments, the at least one cytokine molecule or a functional fragment or variant thereof comprises IL-2 or a functional fragment or variant thereof, IL-7 or a functional fragment or variant thereof, IL-12 or a functional fragment or variant thereof, IL-15 or a functional fragment or variant thereof, IL-18 or a functional fragment or variant thereof, IL-21 or a functional fragment or variant thereof, interferon gamma or a functional fragment or variant thereof, or any combination thereof.

In some embodiments, the at least one cytokine molecule or a functional fragment or variant thereof comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the cytokine sequences listed in Tables 40, 42C, 43A, 43B, and 46-51.

In some embodiments, the at least one cytokine molecule or a functional fragment or variant thereof comprises any one sequence selected from the group consisting of the cytokine sequences listed in Tables 40, 42C, 43A, 43B, and 46-51.

In some embodiments, the at least one cytokine molecule or a functional fragment or variant thereof comprises IL-2 or a functional fragment or variant thereof.

In some embodiments, the IL-2 or a functional fragment or variant thereof comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 2191, the sequence of SEQ ID NO: 2270, or the sequence of SEQ ID NO: 2280.

In some embodiments, the IL-2 or a functional fragment or variant thereof comprises a sequence the sequence of SEQ ID NO: 2191, the sequence of SEQ ID NO: 2270, or the sequence of SEQ ID NO: 2280.

In some embodiments, the at least one cytokine molecule or a functional fragment or variant thereof IL-15 or a functional fragment or variant thereof.

In some embodiments, the IL-15 or a functional fragment or variant thereof comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 2170, SEQ ID NO: 3670, or SEQ ID NO: 3673.

In some embodiments, the IL-15 or a functional fragment or variant thereof comprises the sequence of SEQ ID NO: 2170, the sequence of SEQ ID NO: 3670, or the sequence of SEQ ID NO: 3673.

In some embodiments, the at least one cytokine molecule or a functional fragment or variant thereof further comprises an IL15Ralpha dimerizing domain covalently linked the IL-15 or a functional fragment or variant thereof.

In some embodiments, the IL15Ralpha dimerizing domain comprises an IL-15 receptor alpha sushi domain or a functional fragment or variant thereof.

In some embodiments, the IL15Ralpha dimerizing domain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 3472.

In some embodiments, the IL15Ralpha dimerizing domain comprises the sequence of SEQ ID NO: 3472.

In some embodiments, the IL-15 or a functional fragment or variant thereof is covalently linked to the IL15Ralpha dimerizing domain via a linker.

In some embodiments, the IL-15 or a functional fragment or variant thereof is covalently linked to the IL15Ralpha dimerizing domain via a linker comprising the sequence of SEQ ID NO: 3473.

In some embodiments, the at least one cytokine molecule or a functional fragment or variant thereof comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 3474, SEQ ID NO: 3671, or SEQ ID NO: 8689.

In some embodiments, the at least one cytokine molecule or a functional fragment or variant thereof comprises the sequence of SEQ ID NO: 3474, the sequence of SEQ ID NO: 3671, or the sequence of SEQ ID NO: 8689.

In some embodiments, the at least one cytokine molecule or a functional fragment or variant thereof comprises IL-7 or a functional fragment or variant thereof.

In some embodiments, the IL-7 or the functional fragment or variant thereof comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 3540.

In some embodiments, the IL-7 or the functional fragment or variant thereof comprises the sequence of SEQ ID NO: 3540.

In some embodiments, the at least one cytokine molecule or a functional fragment or variant thereof comprises IL-12 or a functional fragment or variant or variant thereof.

In some embodiments, the IL-12 or the functional fragment or variant thereof comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 3542, the sequence of SEQ ID NO: 3543, or the sequence of SEQ ID NO: 3545.

In some embodiments, the IL-12 or the functional fragment or variant thereof comprises the sequence of SEQ ID NO: 3542, the sequence of SEQ ID NO: 3543, or the sequence of SEQ ID NO: 3545.

In some embodiments, the at least one cytokine molecule or a functional fragment or variant thereof comprises IL-18 of a functional fragment or variant thereof.

In some embodiments, the IL-18 or the functional fragment or variant thereof comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 2192.

In some embodiments, the IL-18 or the functional fragment or variant thereof comprises the sequence of SEQ ID NO: 2192.

In some embodiments, the at least one cytokine molecule or a functional fragment or variant thereof comprises IL-21 or a functional fragment or variant thereof.

In some embodiments, the IL-21 or the functional fragment or variant thereof comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 3540.

In some embodiments, the IL-21 or the functional fragment or variant thereof comprises the sequence of SEQ ID NO: 3540.

In some embodiments, the agent comprises an antigen binding domain that binds to TRBC.

In some embodiments, the antigen binding domain that binds to TRBC binds to TRBC1 or TRBC2.

In some embodiments, the antigen binding domain that binds to TRBC binds to TRBC1, and wherein the antigen binding domain that binds to TRBC comprises a VH comprising:
(i) a VHCDR1 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VHCDR1 sequences listed in Tables 2A, 2B, 3A, 3B, 5A, 5B, 7, 8, 16, 39, and 40;
(ii) a VHCDR2 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity any one sequence selected from the group consisting of the VHCDR2 sequences listed in Tables 2A, 2B, 3A, 3B, 5A, 5B, 7, 8, 16, 39, and 40;
(iii) a VHCDR3 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VHCDR3 sequences listed in Tables 2A, 2B, 3A, 3B, 5A, 5B, 7, 8, 16, 39, and 40; or
(iv) any combination thereof.

In some embodiments, the antigen binding domain that binds to TRBC binds to TRBC1, and wherein the antigen binding domain that binds to TRBC comprises a VH comprising:
(i) a VHCDR1 comprising to any one sequence selected from the group consisting of the VHCDR1 sequences listed in Tables 2A, 2B, 3A, 3B, 5A, 5B, 7, 8, 16, 39, and 40;
(ii) a VHCDR2 comprising any one sequence selected from the group consisting of the VHCDR2 sequences listed in Tables 2A, 2B, 3A, 3B, 5A, 5B, 7, 8, 16, 39, and 40;
(iii) a VHCDR3 comprising any one sequence selected from the group consisting of the VHCDR3 sequences listed in Tables 2A, 2B, 3A, 3B, 5A, 5B, 7, 8, 16, 39, and 40; or
(iv) any combination thereof.

In some embodiments, the antigen binding domain that binds to TRBC binds to TRBC1, and wherein the antigen binding domain that binds to TRBC comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VH sequences listed in Tables 7, 8, 16, 39, and 40.

In some embodiments, the antigen binding domain that binds to TRBC binds to TRBC1, and wherein the antigen binding domain that binds to TRBC comprises a VH comprising any one sequence selected from the group consisting of the VH sequences listed in Tables 7, 8, 16, 39, and 40.

In some embodiments, the antigen binding domain that binds to TRBC binds to TRBC1, and wherein the antigen binding domain that binds to TRBC comprises a VL comprising:
(i) a VLCDR1 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VLCDR1 sequences listed in Tables 4, 6, 7, 8, 16, 39, and 40;
(ii) a VLCDR2 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity any one sequence selected from the group consisting of the VLCDR2 sequences listed in Tables 4, 6, 7, 8, 16, 39, and 40;
(iii) a VLCDR3 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VLCDR3 sequences listed in Tables 4, 6, 7, 8, 16, 39, and 40; or
(iv) any combination thereof.

In some embodiments, the antigen binding domain that binds to TRBC binds to TRBC1, and wherein the antigen binding domain that binds to TRBC comprises a VL comprising:
(i) a VLCDR1 comprising any one sequence selected from the group consisting of the VLCDR1 sequences listed in Tables 4, 6, 7, 8, 16, 39, and 40;
(ii) a VLCDR2 comprising any one sequence selected from the group consisting of the VLCDR2 sequences listed in Tables 4, 6, 7, 8, 16, 39, and 40;
(iii) a VLCDR3 comprising any one sequence selected from the group consisting of the VLCDR3 sequences listed in Tables 4, 6, 7, 8, 16, 39, and 40; or
(iv) any combination thereof.

In some embodiments, the antigen binding domain that binds to TRBC binds to TRBC1, and wherein the antigen binding domain that binds to TRBC comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VL sequences listed in Tables 7, 8, 16, 39, and 40.

In some embodiments, the antigen binding domain that binds to TRBC binds to TRBC1, and wherein the antigen binding domain that binds to TRBC comprises a VL comprising any one sequence selected from the group consisting of the VL sequences listed in Tables 7, 8, 16, 39, and 40.

In some embodiments, the antigen binding domain that binds to TRBC binds to TRBC2, and wherein the antigen binding domain that binds to TRBC comprises a VH comprising:
(i) a VHCDR1 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VHCDR1 sequences listed in Tables 9A, 9B, 11, 12, 14, 15, 17, 39, and 40;
(ii) a VHCDR2 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity any one sequence selected from the group consisting of the VHCDR2 sequences listed in Tables 9A, 9B, 12, 14, 15, 17, 39, and 40;
(iii) a VHCDR3 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VHCDR3 sequences listed in Tables 9A, 9B, 12, 14, 14, 15, 17, 39, and 40; or
(iv) any combination thereof.

In some embodiments, the antigen binding domain that binds to TRBC binds to TRBC2, and wherein the antigen binding domain that binds to TRBC comprises a VH comprising:
(i) a VHCDR1 comprising to any one sequence selected from the group consisting of the VHCDR1 sequences listed in Tables 9A, 9B, 12, 14, 15, 17, 39, and 40;
(ii) a VHCDR2 comprising any one sequence selected from the group consisting of the VHCDR2 sequences listed in Tables 9A, 9B, 12, 14, 15, 17, 39, and 40;
(iii) a VHCDR3 comprising any one sequence selected from the group consisting of the VHCDR3 sequences listed in Tables 9A, 9B, 12, 14, 15, 17, 39, and 40; or (iv) any combination thereof.

In some embodiments, the antigen binding domain that binds to TRBC binds to TRBC2, and wherein the antigen binding domain that binds to TRBC comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VH sequences listed in Tables 11, 15, 17, 39, and 40.

In some embodiments, the antigen binding domain that binds to TRBC binds to TRBC2, and wherein the antigen binding domain that binds to TRBC comprises a VH comprising any one sequence selected from the group consisting of the VH sequences listed in Tables 11, 15, 17, 39, and 40.

In some embodiments, the antigen binding domain that binds to TRBC binds to TRBC2, and wherein the antigen binding domain that binds to TRBC comprises a VL comprising:
(i) a VLCDR1 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VLCDR1 sequences listed in Tables 10, 12, 14, 15, 17, 39, and 40;
(ii) a VLCDR2 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity any one sequence selected from the group consisting of the VLCDR2 sequences listed in Tables 10, 12, 14, 15, 17, 39, and 40;
(iii) a VLCDR3 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VLCDR3 sequences listed in Tables 10, 12, 14, 15, 17, 39, and 40; or
(iv) any combination thereof.

In some embodiments, the antigen binding domain that binds to TRBC binds to TRBC2, and wherein the antigen binding domain that binds to TRBC comprises a VL comprising:
(i) a VLCDR1 comprising any one sequence selected from the group consisting of the VLCDR1 sequences listed in Tables 10, 12, 14, 15, 17, 39, and 40;
(ii) a VLCDR2 comprising any one sequence selected from the group consisting of the VLCDR2 sequences listed in Tables 10, 12, 14, 15, 17, 39, and 40;
(iii) a VLCDR3 comprising any one sequence selected from the group consisting of the VLCDR3 sequences listed in Tables 10, 12, 14, 15, 17, 39, and 40; or
(iv) any combination thereof.

In some embodiments, the antigen binding domain that binds to TRBC binds to TRBC2, and wherein the antigen binding domain that binds to TRBC comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VL sequences listed in Tables 11, 15, 17, 39, and 40.

In some embodiments, the antigen binding domain that binds to TRBC binds to TRBC2, and wherein the antigen binding domain that binds to TRBC comprises a VL comprising any one sequence selected from the group consisting of the VL sequences listed in Tables 11, 15, 17, 39, and 40.

In some embodiments, the antigen binding domain that binds to TRBC binds to TRBC2, and wherein the antigen binding domain that binds to TRBC comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the scFv sequences listed in Tables 12, 15, 17, 39, and 40.

In some embodiments, the antigen binding domain that binds to TRBC binds to TRBC2, and wherein the antigen binding domain that binds to TRBC comprises a VL comprising any one sequence selected from the group consisting of the scFv sequences listed in Tables 12, 15, 17, 39, and 40.

In some embodiments, the agent comprises a second antigen binding domain that binds to NKp30.

In some embodiments, the antigen binding domain and the second antigen binding domain that binds to NKp30 are same.

In some embodiments, the antigen binding domain and the second antigen binding domain that binds to NKp30 are different.

In some embodiments, the second antigen binding domain that binds to NKp30 comprises a heavy chain variable region (VH) comprising:
(i) a heavy chain complementarity determining region 3 (VHCDR3) comprising the sequence of GDWHYFDY (SEQ ID NO: 504); and
(ii) a heavy chain complementarity determining region 1 (VHCDR1) comprising the sequence of GFSIIRVGYHWN (SEQ ID NO: 502), a heavy chain complementarity determining region 2 (VHCDR2) comprising the sequence of YIHRSGSTSYNPSLKS (SEQ ID NO: 503), or a combination thereof.

In some embodiments, the second antigen binding domain that binds to NKp30 comprises a VH comprising the VHCDR1 comprising the sequence of GFSIIRVGYHWN (SEQ ID NO: 502), the VHCDR2 comprising the sequence of YIHRSGSTSYNPSLKS (SEQ ID NO: 503), and the VHCDR3 comprising the sequence of GDWHYFDY (SEQ ID NO: 504).

In some embodiments, the second antigen binding domain that binds to NKp30 comprises a light chain variable region (VL) comprising a light chain complementarity determining region 1 (VLCDR1) comprising the sequence of SGEKLSDKYVH (SEQ ID NO: 26), a light chain complementarity determining region 2 (VLCDR2) comprising the sequence of ENDRRPS (SEQ ID NO: 28), a light chain complementarity determining region 3 (VLCDR3) comprising the sequences of QFWDSTNSAV (SEQ ID NO: 86), or any combination thereof.

In some embodiments, the second antigen binding domain that binds to NKp30 comprises a VL comprising a VLCDR1 comprising the sequence of SGEKLSDKYVH (SEQ ID NO: 26), a VLCDR2 comprising the sequence of ENDRRPS (SEQ ID NO: 28), and a VLCDR3 comprising the sequences of QFWDSTNSAV (SEQ ID NO: 86).

In some embodiments, the second antigen binding domain that binds to NKp30 comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 505.

In some embodiments, the second antigen binding domain that binds to NKp30 comprises a VH comprising the sequence of SEQ ID NO: 505.

In some embodiments, the second antigen binding domain that binds to NKp30 comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 13.

In some embodiments, the second antigen binding domain that binds to NKp30 comprises a VL comprising the sequence of SEQ ID NO: 13.

In some embodiments, the second antigen binding domain that binds to NKp30 comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 505 and a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 13.

In some embodiments, the second antigen binding domain that binds to NKp30 comprises a VH comprising the sequence of SEQ ID NO: 505 and a VL comprising the sequence of SEQ ID NO: 13.

In some embodiments, the second antigen binding domain that binds to NKp30 comprises a heavy chain variable region (VH) comprising:
(i) a VHCDR1 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VHCDR1 sequences listed in Tables 20A, 20B, 21A, 21B, 23A, 23B, 25, 26, 16, and 17;
(ii) a VHCDR2 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity any one sequence selected from the group consisting of the VHCDR2 sequences listed in Tables 20A, 20B, 21A, 21B, 23A, 23B, 25, 26, 16, and 17;
(iii) a VHCDR3 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VHCDR3 sequences listed in Tables 20A, 20B, 21A, 21B, 23A, 23B, 25, 26, 16, and 17; or
(iv) any combination thereof.

In some embodiments, the second antigen binding domain that binds to NKp30 comprises a VH comprising:
(i) a VHCDR1 comprising to any one sequence selected from the group consisting of the VHCDR1 sequences listed in Tables 20A, 20B, 21A, 21B, 23A, 23B, 25, 26, 16, and 17;
(ii) a VHCDR2 comprising any one sequence selected from the group consisting of the VHCDR2 sequences listed in Tables 20A, 20B, 21A, 21B, 23A, 23B, 25, 26, 16, and 17;
(iii) a VHCDR3 comprising any one sequence selected from the group consisting of the VHCDR3 sequences listed in Tables 20A, 20B, 21A, 21B, 23A, 23B, 25, 26, 16, and 17; or
(iv) any combination thereof.

In some embodiments, the second antigen binding domain that binds to NKp30 comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VH sequences listed in Tables 25, 26, 16, and 17.

In some embodiments, the second antigen binding domain that binds to NKp30 comprises a VH comprising any one sequence selected from the group consisting of the VH sequences listed in Tables 25, 26, 16, and 17.

In some embodiments, the second antigen binding domain that binds to NKp30 comprises a VL comprising:
(i) a VLCDR1 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VLCDR1 sequences listed in Tables 22, 24, 25, 26, 16, and 17;
(ii) a VLCDR2 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity any one sequence selected from the group consisting of the VLCDR2 sequences listed in Tables 22, 24, 25, 26, 16, and 17;
(iii) a VLCDR3 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VLCDR3 sequences listed in Tables 22, 24, 25, 26, 16, and 17; or
(iv) any combination thereof.

In some embodiments, the second antigen binding domain that binds to NKp30 comprises a VL comprising:
(i) a VLCDR1 comprising any one sequence selected from the group consisting of the VLCDR1 sequences listed in Tables 22, 24, 25, 26, 16, and 17;
(ii) a VLCDR2 comprising any one sequence selected from the group consisting of the VLCDR2 sequences listed in Tables 22, 24, 25, 26, 16, and 17;
(iii) a VLCDR3 comprising any one sequence selected from the group consisting of the VLCDR3 sequences listed in Tables 22, 24, 25, 26, 16, and 17; or
(iv) any combination thereof.

In some embodiments, the second antigen binding domain that binds to NKp30 comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VL sequences listed in Tables 25, 26, 16, and 17.

In some embodiments, the second antigen binding domain that binds to NKp30 comprises a VL comprising any one sequence selected from the group consisting of the VL sequences listed in Tables 25, 26, 16, and 17.

In some embodiments, the agent comprises an immune cell engager.

In some embodiments, the immune cell engager is selected from the group consisting of a T cell engager, an NK cell engager, a B cell engager, a dendritic cell engager, and the macrophage cell engager.

In some embodiments, the agent comprises a T cell engager.

In some embodiments, the T cell engager comprises an antigen binding domain that binds to a T cell receptor beta chain region (TCRβV).

In some embodiments, the antigen binding domain that binds to a T cell receptor beta chain region (TCRβV) binds to a TCRβV of any one T cell receptor beta chain (TCRβ) subfamily member selected from the group consisting of TCRβ subfamily members listed in Tables 29 and 42A.

In some embodiments, the antigen binding domain that binds to a TCRβV comprises a heavy chain variable region (VH) comprising:
(i) a VHCDR1 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VHCDR1 sequences listed in Tables 30, 32, 42B, 42C, 43A, 43B, and 43-51;
(ii) a VHCDR2 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity any one sequence selected from the group consisting of the VHCDR2 sequences listed in Tables 30, 32, 42B, 42C, 43A, 43B, and 43-51;
(iii) a VHCDR3 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VHCDR3 sequences listed in Tables 30, 32, 42B, 42C, 43A, 43B, and 43-51; or
(iv) any combination thereof.

In some embodiments, the antigen binding domain that binds to a TCRβV comprises a VH comprising:
(i) a VHCDR1 comprising to any one sequence selected from the group consisting of the VHCDR1 sequences listed in Tables 30, 32, 42B, 42C, 43A, 43B, and 43-51; (ii) a VHCDR2 comprising any one sequence selected from the group consisting of the VHCDR2 sequences listed in Tables 30, 32, 42B, 42C, 43A, 43B, and 43-51;
(iii) a VHCDR3 comprising any one sequence selected from the group consisting of the VHCDR3 sequences listed in Tables 30, 32, 42B, 42C, 43A, 43B, and 43-51; or
(iv) any combination thereof.

In some embodiments, the antigen binding domain that binds to a TCRβV comprises a VH comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VH sequences listed in Tables 30, 32, 42B, 42C, 43A, 43B, and 43-51.

In some embodiments, the antigen binding domain that binds to a TCRβV comprises a VH comprising any one sequence selected from the group consisting of the VH sequences listed in Tables 30, 32, 42B, 42C, 43A, 43B, and 43-51.

In some embodiments, the antigen binding domain that binds to a TCRβV comprises a VL comprising:
(i) a VLCDR1 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VLCDR1 sequences listed in Tables 30, 32, 42B, 42C, 43A, 43B, and 43-51;
(ii) a VLCDR2 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity any one sequence selected from the group consisting of the VLCDR2 sequences listed in Tables 30, 32, 42B, 42C, 43A, 43B, and 43-51;
(iii) a VLCDR3 comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VLCDR3 sequences listed in Tables 30, 32, 42B, 42C, 43A, 43B, and 43-51; or
(iv) any combination thereof.

In some embodiments, the antigen binding domain that binds to a TCRβV comprises a VL comprising:
(i) a VLCDR1 comprising any one sequence selected from the group consisting of the VLCDR1 sequences listed in Tables 30, 32, 42B, 42C, 43A, 43B, and 43-51;
(ii) a VLCDR2 comprising any one sequence selected from the group consisting of the VLCDR2 sequences listed in Tables 30, 32, 42B, 42C, 43A, 43B, and 43-51;
(iii) a VLCDR3 comprising any one sequence selected from the group consisting of the VLCDR3 sequences listed in Tables 30, 32, 42B, 42C, 43A, 43B, and 43-51; or
(iv) any combination thereof.

In some embodiments, the antigen binding domain that binds to a TCRβV comprises a VL comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the VL sequences listed in Tables 30, 32, 42B, 42C, 43A, 43B, and 43-51.

In some embodiments, the antigen binding domain that binds to a TCRβV comprises a VL comprising any one sequence selected from the group consisting of the VL sequences listed in Tables 30, 32, 42B, 42C, 43A, 43B, and 43-51.

In some embodiments, the agent comprises a NK cell engager.

In some embodiments, the NK cell engager does not bind to NKp30.

In some embodiments, the NK cell engager comprise a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the sequences listed in Table 27.

In some embodiments, the NK cell engager comprise any one sequence selected from the group consisting of the sequences listed in Table 27.

In some embodiments, the agent comprises a tumor-targeting moiety.

In some embodiments, the tumor-targeting moiety, e.g., cancer antigen, is selected from the group consisting of BCMA, FcRH5, CD19, CD20, CD22, CD30, CD33, CD38, CD47, CD99, CD123, DLL3, CLDN18.2, FcRH5, CLEC12, CD179A, SLAMF7, or NY-ESO1, PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, a actinin-4, TRP1/gp75, TRP2, gp100, Melan-A/MART1, gangliosides, WT1, EphA3, Epidermal growth factor receptor (EGFR), MART-2, MART-1, MUC1, MUC2, MUM1, MUM2, MUM3, NA88-1, NPM, OAl, OGT, RCC, RUl1, RUl2, SAGE, TRG, TRP1, TSTA, Folate receptor alpha, L1-CAM, CAIX, EGFRvIII, gpA33, GD3, GM2, VEGFR, Intergrins (Integrin alphaVbeta3, Integrin alpha5Beta1), Carbohydrates (Le), IGF1R, EPHA3, TRAILR1, TRAILR2, RANKL, FAP, TGF-beta, hyaluronic acid, collagen, e.g., collagen IV, tenascin C, and tenascin W.

In some embodiments, the tumor-targeting moiety, e.g., cancer antigen, is chosen from: CD19, CD123, DLL3, CLDN18.2, CD22, CD30, CD171, CS-1, C-type lectin-like molecule-1, CD33, epidermal growth factor receptor variant III (EGFRvIII), ganglioside G2 (GD2), ganglioside GD3, TNF receptor family member B cell maturation (BCMA), Tn antigen ((Tn Ag) or (GalNAcα-Ser/Thr)), prostate-specific membrane antigen (PSMA), Receptor tyrosine kinase-like orphan receptor 1 (RORI), Fms-Like Tyrosine Kinase 3 (FLT3), Tumor-associated glycoprotein 72 (TAG72), CD38, CD44v6, Carcinoembryonic antigen (CEA), Epithelial cell adhesion molecule (EPCAM), B7H3 (CD276), KIT (CD117), Interleukin-13 receptor subunit alpha-2, mesothelin, Interleukin 11 receptor alpha (IL-11Ra), prostate stem cell antigen (PSCA), Protease Serine 21, vascular endothelial growth factor receptor 2 (VEGFR2), Lewis(Y) antigen, CD24, Platelet-derived growth factor receptor beta (PDGFR-beta), Stage-specific embryonic antigen-4 (SSEA-4), CD20, Folate receptor alpha, Receptor tyrosine-protein kinase ERBB2 (Her2/neu), Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-I receptor), carbonic anhydrase IX (CAIX), Proteasome (Prosome, Macropain) Subunit, Beta Type, 9 (LMP2), glycoprotein 100 (gp100), oncogene fusion protein consisting of breakpoint cluster region (BCR) and Abelson murine leukemia viral oncogene homolog 1 (Abl) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2), Folate receptor beta, tumor endothelial marker 1 (TEM1/CD248), tumor endothelial marker 7-related (TEM7R), claudin 6 (CLDN6), thyroid stimulating hormone receptor (TSHR), G protein-coupled receptor class C group 5, member D (GPRC5D), chromosome X open reading frame 61 (CXORF61), CD97, CD179a, anaplastic lymphoma kinase (ALK), Polysialic acid, placenta-specific 1 (PLAC1), hexasaccharide portion of globoH glycoceramide (GloboH), mammary gland differentiation antigen (NY-BR-1), uroplakin 2 (UPK2), Hepatitis A virus cellular receptor 1 (HAVCRI), adrenoceptor beta 3 (ADRB3), pannexin 3 (PANX3), G protein-coupled receptor 20 (GPR20), lymphocyte antigen 6 complex, locus K 9 (LY6K), Olfactory receptor 51E2 (OR51E2), TCR Gamma Alternate Reading Frame Protein (TARP), Wilms tumor protein (WT1), Cancer/testis antigen 1 (NY-ESO-1), Cancer/testis antigen 2 (LAGE-la), Melanoma-associated antigen 1 (MAGE-A1), ETS translocation-variant gene 6, located on chromosome 12p (ETV6-AML), sperm protein 17 (SPA17), X Antigen Family, Member 1A (XAGE1), angiopoietin-binding cell surface receptor 2 (Tie 2), melanoma cancer testis antigen-1 (MAD-CT-1), melanoma cancer testis antigen-2 (MAD-CT-2), Fos-related antigen 1, tumor protein p53 (p53), p53 mutant, prostein, surviving, telomerase, prostate carcinoma tumor antigen-1, melanoma antigen recognized by T cells 1, Rat sarcoma (Ras) mutant, human Telomerase reverse transcriptase (hTERT), sarcoma translocation breakpoints, melanoma inhibitor of apoptosis (ML-IAP), ERG (transmembrane protease, serine 2 (TMPRSS2) ETS fusion gene), N-Acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), Tyrosinase-related protein 2 (TRP-2), Cytochrome P450 1B1 (CYP1B1), CCCTC-Binding Factor (Zinc Finger Protein)-Like, Squamous Cell Carcinoma Antigen Recognized By T Cells 3 (SART3), Paired box protein Pax-5 (PAX5), proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), renal ubiquitous 2 (RU2), legumain, human papilloma virus E6 (HPV E6), human papilloma virus E7 (HPV E7), intestinal carboxyl esterase, heat shock protein 70-2 mutated (mut hsp70-2), CD79a, CD79b, CD72, Leukocyte-associated immunoglobulin-like receptor 1 (LAIRI), Fc fragment of IgA receptor (FCAR or CD89), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), or immunoglobulin lambda-like polypeptide 1 (IGLL1).

In some embodiments, the antigen binding domain that binds TRBC, the second antigen binding domain that binds to NKp30, the immune cell engager, the tumor-targeting moiety, or any combination thereof is a full antibody or antigen-binding fragment thereof, a bivalent antibody, bispecific antibody, or a biparatopic antibody.

In some embodiments, the antigen binding domain that binds TRBC, the second antigen binding domain that binds to NKp30, the immune cell engager, the tumor-targeting moiety, or any combination thereof is a Fab, F(ab′)2, Fv, an single chain Fv (scFv), a diabody, a single domain antibody, or a camelid antibody.

In some embodiments, the antigen binding domain that binds TRBC, the second antigen binding domain that binds to NKp30, the immune cell engager, the tumor-targeting moiety, or any combination thereof is a Fab or an scFv.

In some embodiments, the antigen binding domain that binds TRBC, the second antigen binding domain that binds to NKp30, the immune cell engager, the tumor-targeting moiety, or any combination thereof comprises one or more heavy chain constant regions selected from the group consisting of IgG1 heavy chain constant region or functional fragment thereof, IgG2 heavy chain constant region or functional fragment thereof, IgG3 heavy chain constant region or functional fragment thereof, IgGA1 heavy chain constant region or functional fragment thereof, IgGA2 heavy chain constant region or functional fragment thereof, IgG4 heavy chain constant region or functional fragment thereof, IgJ heavy chain constant region or functional fragment thereof, IgM heavy chain constant region or functional fragment thereof, IgD heavy chain constant region or functional fragment thereof, and IgE heavy chain constant region or functional fragment thereof.

In some embodiments, the one or more heavy chain constant regions comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one heavy chain constant region sequences listed in Tables 3, 7, 8, 16, 17, 39, 40, 42C, 43A, 43B, and 46-51.

In some embodiments, the one or more heavy chain constant regions comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 73, the sequence of SEQ ID NO: 76, the sequence of SEQ ID NO: 74, the sequence of SEQ ID NO: 75, the sequence of SEQ ID NO: 76, the sequence of SEQ ID NO: 40, the sequence of SEQ ID NO: 41, the sequence of SEQ ID NO: 42, the sequence of SEQ ID NO: 3645, the sequence of SEQ ID NO: 3646, the sequence of SEQ ID NO: 3649, the sequence of SEQ ID NO: 3451, the sequence of SEQ ID NO: 506, the sequence of SEQ ID NO: 507, the sequence of SEQ ID NO: 3647, the sequence of SEQ ID NO: 3648, the sequence of SEQ ID NO: 508 or 8686, the sequence of SEQ ID NO: 513, the sequence of SEQ ID NO: 514, the sequence of SEQ ID NO: 515, the sequence of SEQ ID NO: 516, the sequence of SEQ ID NO: 3447, or the sequence of SEQ ID NO: 511.

In some embodiments, the antigen binding domain that binds TRBC, the second antigen binding domain that binds to NKp30, the immune cell engager, the tumor-targeting moiety, or any combination thereof comprises a kappa light chain constant region or functional fragment thereof, a lambda light chain constant region or functional fragment thereof, or a combination thereof.

In some embodiments, the kappa light chain constant region comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one light chain constant region sequences listed in Tables 3, 7, 8, 16, 17, 39, 40, 42C, 43A, 43B, and 46-51.

In some embodiments, the kappa light chain constant region comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 39 or the sequence of SEQ ID NO: 3644.

In some embodiments, the first member of the dimerization module comprises a first Fc region and the second member of the dimerization module comprises a second Fc region.

In some embodiments, the first Fc region is selected from the group consisting of an IgG1 Fc region or a functional fragment thereof, an IgG2 Fc region or a functional fragment thereof, an IgG3 Fc region or a functional fragment thereof, an IgGA1 Fc region or a functional fragment thereof, an IgGA2 Fc region or a functional fragment thereof, an IgG4 Fc region or a functional fragment thereof, an IgJ Fc region or a functional fragment thereof, an IgM Fc region or a functional fragment thereof, an IgD Fc region or a functional fragment thereof, and an IgE Fc region or a functional fragment thereof.

In some embodiments, the second Fc region is selected from the group consisting of an IgG1 Fc region or a functional fragment thereof, an IgG2 Fc region or a functional fragment thereof, an IgG3 Fc region or a functional fragment thereof, an IgGA1 Fc region or a functional fragment thereof, an IgGA2 Fc region or a functional fragment thereof, an IgG4 Fc region or a functional fragment thereof, an IgJ Fc region or a functional fragment thereof, an IgM Fc region or a functional fragment thereof, an IgD Fc region or a functional fragment thereof, and an IgE Fc region or a functional fragment thereof.

In some embodiments, the first Fc region, the second Fc region, or a combination thereof comprises an Fe interface with one or more of: a paired cavity-protuberance, an electrostatic interaction, or a strand-exchange, wherein the dimerization of the first Fc region and the second Fc region is enhanced as indicated by a greater ratio of heteromultimer:homomultimer forms relative to a dimerization of Fc regions with a non-engineered interface.

In some embodiments,
(i) the first Fc region comprises:
(a) an Ala at position 297 in a heavy chain constant region according to EU Numbering
(b) a Cys at position 349 in a heavy chain constant region according to EU Numbering),
(c) a Ser at position 366 in a heavy chain constant region according to EU Numbering),
(d) an Ala at position 368 in a heavy chain constant region according to EU Numbering); and
(e) a Val at position 407 in a heavy chain constant region according to EU Numbering); and
(ii) the second Fc region comprises:
(a) an Ala at position 297 in a heavy chain constant region according to EU Numbering),
(b) a Cys at position 354 in a heavy chain constant region according to EU Numbering), and
(c) a Trp at position 366 in a heavy chain constant region according to EU Numbering).

In some embodiments,
(i) the first Fc region comprises:
(a) an Ala at position 297 in a heavy chain constant region according to EU Numbering),
(b) a Cys at position 354 in a heavy chain constant region according to EU Numbering), and
(c) a Trp at position 366 in a heavy chain constant region according to EU Numbering); and
(ii) the second Fc region comprises:
(a) an Ala at position 297 in a heavy chain constant region according to EU Numbering
(b) a Cys at position 349 in a heavy chain constant region according to EU Numbering),
(c) a Ser at position 366 in a heavy chain constant region according to EU Numbering),
(d) an Ala at position 368 in a heavy chain constant region according to EU Numbering); and
(e) a Val at position 407 in a heavy chain constant region according to EU Numbering).

In some embodiments, the first Fc region comprises a mutation that decreases Fc receptor binding to the agent relative to an agent without the mutation.

In some embodiments, the first Fc region comprises an Asn297Ala (N297A) mutation or a Leu234Ala/Leu235Ala (LALA) mutation, or a combination thereof according to EU Numbering.

In some embodiments, the second Fc region comprises a mutation that decreases Fc receptor binding to the agent relative to an agent without the mutation.

In some embodiments, the second Fc region comprises an Asn297Ala (N297A) mutation or a Leu234Ala/Leu235Ala (LALA) mutation, or a combination thereof according to EU Numbering.

In some embodiments, the first Fc region comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the sequence of SEQ ID NO: 73, the sequence of SEQ ID NO: 76, the sequence of SEQ ID NO: 74, the sequence of SEQ ID NO: 75, the sequence of SEQ ID NO: 40, the sequence of SEQ ID NO: 41, the sequence of SEQ ID NO: 42, the sequence of SEQ ID NO: 3645, the sequence of SEQ ID NO: 3646, the sequence of SEQ ID NO: 3649, the sequence of SEQ ID NO: 8687, the sequence of SEQ ID NO: 506, the sequence of SEQ ID NO: 507, the sequence of SEQ ID NO: 3647, the sequence of SEQ ID NO: 3648, the sequence of SEQ ID NO: 508 or 8686, the sequence of SEQ ID NO: 513, the sequence of SEQ ID NO: 514, the sequence of SEQ ID NO: 515, the sequence of SEQ ID NO: 516, the sequence of SEQ ID NO: 3447, and the sequence of SEQ ID NO: 511.

In some embodiments, the second Fc region comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the sequence of SEQ ID NO: 73, the sequence of SEQ ID NO: 76, the sequence of SEQ ID NO: 74, the sequence of SEQ ID NO: 75, the sequence of SEQ ID NO: 40, the sequence of SEQ ID NO: 41, the sequence of SEQ ID NO: 42, the sequence of SEQ ID NO: 3645, the sequence of SEQ ID NO: 3646, the sequence of SEQ ID NO: 3649, the sequence of SEQ ID NO: 8687, the sequence of SEQ ID NO: 506, the sequence of SEQ ID NO: 507, the sequence of SEQ ID NO: 3647, the sequence of SEQ ID NO: 3648, the sequence of SEQ ID NO: 508 or 8686, the sequence of SEQ ID NO: 513, the sequence of SEQ ID NO: 514, the sequence of SEQ ID NO: 515, the sequence of SEQ ID NO: 516, the sequence of SEQ ID NO: 3447, and the sequence of SEQ ID NO: 511.

In some embodiments, the first Fc region comprises any one sequence selected from the group consisting of the sequence of SEQ ID NO: 73, the sequence of SEQ ID NO: 76, the sequence of SEQ ID NO: 74, the sequence of SEQ ID NO: 75, the sequence of SEQ ID NO: 40, the sequence of SEQ ID NO: 41, the sequence of SEQ ID NO: 42, the sequence of SEQ ID NO: 3645, the sequence of SEQ ID NO: 3646, the sequence of SEQ ID NO: 3649, the sequence of SEQ ID NO: 8687, the sequence of SEQ ID NO: 506, the sequence of SEQ ID NO: 507, the sequence of SEQ ID NO: 3647, the sequence of SEQ ID NO: 3648, the sequence of SEQ ID NO: 508 or 8686, the sequence of SEQ ID NO: 513, the sequence of SEQ ID NO: 514, the sequence of SEQ ID NO: 515, the sequence of SEQ ID NO: 516, the sequence of SEQ ID NO: 3447, and the sequence of SEQ ID NO: 511.

In some embodiments, the second Fc region comprises any one sequence selected from the group consisting of the sequence of SEQ ID NO: 73, the sequence of SEQ ID NO: 76, the sequence of SEQ ID NO: 74, the sequence of SEQ ID NO: 75, the sequence of SEQ ID NO: 40, the sequence of SEQ ID NO: 41, the sequence of SEQ ID NO: 42, the sequence of SEQ ID NO: 3645, the sequence of SEQ ID NO: 3646, the sequence of SEQ ID NO: 3649, the sequence of SEQ ID NO: 8687, the sequence of SEQ ID NO: 506, the sequence of SEQ ID NO: 507, the sequence of SEQ ID NO: 3647, the sequence of SEQ ID NO: 3648, the sequence of SEQ ID NO: 508 or 8686, the sequence of SEQ ID NO: 513, the sequence of SEQ ID NO: 514, the sequence of SEQ ID NO: 515, the sequence of SEQ ID NO: 516, the sequence of SEQ ID NO: 3447, and the sequence of SEQ ID NO: 511.

In some embodiments, the first Fc region comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the sequence of SEQ ID NO: 3648, the sequence of SEQ ID NO: 508 or 8686, the sequence of SEQ ID NO: 506, the sequence of SEQ ID NO: 507, the sequence of SEQ ID NO: 3477, the sequence of SEQ ID NO: 511, the sequence of SEQ ID NO: 515, and the sequence of SEQ ID NO: 516.

In some embodiments, the second Fc region comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 3648, the sequence of SEQ ID NO: 508 or 8686, the sequence of SEQ ID NO: 506, the sequence of SEQ ID NO: 507, the sequence of SEQ ID NO: 3477, the sequence of SEQ ID NO: 511, the sequence of SEQ ID NO: 515, or the sequence of SEQ ID NO: 516.

In some embodiments, the first Fc region comprises any one sequence selected from the group consisting of the sequence of SEQ ID NO: 3648, SEQ ID NO: 508 or 8686, SEQ ID NO: 506, SEQ ID NO: 507, SEQ ID NO: 3477, SEQ ID NO: 511, SEQ ID NO: 515, or SEQ ID NO: 516.

In some embodiments, the second Fc region comprises any one sequence selected from the group consisting of the sequence of SEQ ID NO: 3648, SEQ ID NO: 508 or 8686, SEQ ID NO: 506, SEQ ID NO: 507, SEQ ID NO: 3477, SEQ ID NO: 511, SEQ ID NO: 515, or SEQ ID NO: 516.

In some embodiments, (i) the first Fc region comprises the sequence of SEQ ID NO: 506 or the sequence of SEQ ID NO: 507, and the second Fc region comprises the sequence of SEQ ID NO: 3648 or the sequence of SEQ ID NO: 508 or 8686; or (ii) the first Fc region comprises the sequence of SEQ ID NO: 3447 or the sequence of SEQ ID NO: 511, and the second Fc region comprises the sequence of SEQ ID NO: 515 or the sequence of SEQ ID NO: 516.

In some embodiments,
(i) the first Fc region comprises the sequence of SEQ ID NO: 3648 or the sequence of SEQ ID NO: 508 or 8686, and the second Fc region comprises the sequence of SEQ ID NO: 506 or the sequence of SEQ ID NO: 507; or
(ii) the first Fc region comprises the sequence of SEQ ID NO: 515 or the sequence of SEQ ID NO: 516, and the second Fc region comprises the sequence of SEQ ID NO: 3447 or the sequence of SEQ ID NO: 511.

In some embodiments, the first Fc region, the second Fc region, or a combination thereof comprises one or more mutations and/or one or more modifications listed in Table 1 and Table 18.

In some embodiments, the agent comprises a first polypeptide, a second polypeptide, and at least one cytokine molecule or a functional fragment or variant thereof, wherein the first polypeptide and the second polypeptide are non-contiguous; wherein:
(i) the first polypeptide comprises a first member of the dimerization module linked to:
(a) the antigen binding domain comprising a VH and a VL, or
(b) a first portion of the antigen binding domain comprising the VH, wherein the agent further comprises a third polypeptide comprising a second portion of the antigen binding domain comprising a VL, wherein the third polypeptide is non-contiguous with the first polypeptide and the second polypeptide; and
(ii) the second polypeptide comprises a second member of the dimerization module, and
wherein the at least one cytokine molecule or a functional fragment or variant thereof is covalently linked to the second polypeptide or the first polypeptide.

In some embodiments, the agent comprises a first polypeptide, a second polypeptide, and at least one cytokine molecule or a functional fragment or variant thereof, wherein the first polypeptide and the second polypeptide are non-contiguous; wherein:
(i) the first polypeptide comprises a first member of a dimerization module linked to a first portion of the antigen binding domain comprising the VH, wherein the agent further comprises a third polypeptide comprising a second portion of the antigen binding domain comprising the VL, wherein the third polypeptide is non-contiguous with the first polypeptide and the second polypeptide; and
(ii) the second polypeptide comprises a second member of the dimerization module, and wherein the at least one cytokine molecule or a functional fragment or variant thereof is covalently linked to the second polypeptide.

In some embodiments, the at least one cytokine molecule or a functional fragment or variant thereof is covalently linked to the N-terminus of the second polypeptide, the C-terminus of the second polypeptide, or a combination thereof.

In some embodiments, the at least one cytokine molecule or a functional fragment or variant thereof is within a single contiguous polypeptide chain of the second polypeptide.

In some embodiments, the first polypeptide comprises:
(i) the antigen binding domain comprising the VH and the VL, wherein the antigen binding domain further comprises a heavy chain constant domain 1 (CH1) linked to the VH; or
(ii) the first portion of the first antigen binding domain comprising the VH, wherein the first portion of the antigen binding domain further comprises a CH1 linked to the VH.

In some embodiments, the CH1 is linked to the C-terminus of the VH.

In some embodiments, the agent comprises:
(i) the first polypeptide comprising the antigen binding domain that comprises the VH and the VL, wherein the antigen binding domain further comprises a light chain constant domain (CL) linked to the VL; or
(ii) the first polypeptide comprising the first portion of the antigen binding domain and the third polypeptide comprising the second portion of the antigen binding domain, wherein the second portion of the antigen binding domain further comprises a CL linked to the VL.

In some embodiments, the CL is linked to the C-terminus of the VL.

In some embodiments, the first member of the dimerization module is linked to the C-terminus of the antigen binding domain comprising the VH and the VL, or the C-terminus of the first portion of the first antigen binding domain comprising the VH.

In some embodiments, the second polypeptide comprises:
(i) an antigen binding domain that binds TRBC, an second antigen binding domain that binds to NKp30, an immune cell engager, or an tumor-targeting moiety comprising a VH and a VL, wherein the antigen binding domain that binds TRBC, the second antigen binding domain that binds to NKp30, the immune cell engager, or the tumor-targeting moiety further comprises a heavy chain constant domain 1 (CH1) linked to the VH; or
(ii) the first portion of the antigen binding domain that binds TRBC, the second antigen binding domain that binds to NKp30, the immune cell engager, or the tumor-targeting moiety comprising the VH, wherein the first portion of the antigen binding domain that binds TRBC, the second antigen binding domain that binds to NKp30, the immune cell engager, or the tumor-targeting moiety further comprises a CH1 linked to the VH.

In some embodiments, the CH1 is linked to the C-terminus of the VH.

In some embodiments, the agent comprises:
(i) the second polypeptide comprising an antigen binding domain that binds TRBC, an second antigen binding domain that binds to NKp30, an immune cell engager, or an tumor-targeting moiety that comprises a VH and a VL, wherein the antigen binding domain that binds TRBC, the second antigen binding domain that binds to NKp30, the immune cell engager, or the tumor-targeting moiety further comprises a light chain constant domain (CL) linked to the VL; or
(ii) the second polypeptide comprising the first portion of the antigen binding domain that binds TRBC, the second antigen binding domain that binds to NKp30, the immune cell engager, or the tumor-targeting moiety,
wherein the agent further comprises a fourth polypeptide comprising the second portion of the antigen binding domain that binds TRBC, the second antigen binding domain that binds to NKp30, the immune cell engager, or the tumor-targeting moiety, wherein the fourth polypeptide is non-contiguous with the first polypeptide, the second polypeptide, and third polypeptide, and
wherein the second portion of the antigen binding domain that binds TRBC, the second antigen binding domain that binds to NKp30, the immune cell engager, or the tumor-targeting moiety further comprises a CL linked to the VL.

In some embodiments, the CL is linked to the C-terminus of the VL.

In some embodiments, the second member of the dimerization module is linked to the C-terminus of the antigen binding domain that binds TRBC, the second antigen binding domain that binds to NKp30, the immune cell engager, or the tumor-targeting moiety comprising the VH and the VL, or the C-terminus of the first portion of the antigen binding domain that binds TRBC, the second antigen binding domain that binds to NKp30, the immune cell engager, or the tumor-targeting moiety comprising the VH.

In some embodiments, the agent further comprises:
(i) a linker between the first member of the dimerization module and the antigen binding domain comprising a VH and a VL, or the first portion of the antigen binding domain comprising a VH;
(ii) a linker between the second member of the dimerization module and an antigen binding domain that binds TRBC, an second antigen binding domain that binds to NKp30, an immune cell engager, or an tumor-targeting moiety comprising a VH and a VL, or the first portion of the antigen binding domain that binds TRBC, the second antigen binding domain that binds to NKp30, the immune cell engager, or the tumor-targeting moiety comprising a VH;
(iii) a linker between the VH and the VL of the antigen binding domain;
(iv) a linker between the CH1 and the VH of the antigen binding domain;
(v) a linker between the CL and the VL of the antigen binding domain;
(vi) a linker between the VH and the VL of the antigen binding domain that binds TRBC, the second antigen binding domain that binds to NKp30, the immune cell engager, or the tumor-targeting moiety;
(vii) a linker between the CH1 and the VH of the antigen binding domain that binds TRBC, the second antigen binding domain that binds to NKp30, the immune cell engager, or the tumor-targeting moiety;
(viii) a linker between the CL and the VL of the antigen binding domain that binds TRBC, the second antigen binding domain that binds to NKp30, the immune cell engager, or the tumor-targeting moiety;
(ix) a linker between the at least one cytokine molecule or a functional fragment or variant thereof and the first polypeptide, a linker between the at least one cytokine molecule or a functional fragment or variant thereof and the second polypeptide, or any combination thereof; or
(x) any combination thereof.

In some embodiments, the linker is selected from the group consisting of a cleavable linker, a non-cleavable linker, a peptide linker, a flexible linker, a rigid linker, a helical linker, and a non-helical linker.

In some embodiments, the linker is the peptide linker comprises the sequence of SEQ ID NO: 3473, SEQ ID NO: 3308, or SEQ ID NO: 8291.

In some embodiments, the at least one cytokine molecule or a functional fragment or variant thereof is linked to the N-terminus of the first polypeptide, the C-terminus of the first polypeptide, the N-terminus of the second polypeptide, the C-terminus of the second polypeptide, the N-terminus of the third polypeptide, the C-terminus of the third polypeptide, or any combination thereof.

In some embodiments, the at least one cytokine molecule or a functional fragment or variant thereof is within a single contiguous polypeptide chain of the first polypeptide, the second polypeptide, or the third cytokine polypeptide to which the at least one cytokine molecule or a functional fragment or variant thereof is linked.

In some embodiments, the agent comprises:
(i) the first polypeptide comprising the first portion of the dimerization module linked to the C-terminus of the first portion of the antigen binding domain;
(ii) the second polypeptide comprising the second portion of the dimerization module;
(iii) the third polypeptide comprising the second portion of the antigen binding domain; and
(iv) the at least one cytokine molecule or a functional fragment or variant thereof covalently linked to the N-terminus of the second polypeptide.

In some embodiments, the agent comprises:
(i) a first polypeptide chain comprising the sequence of SEQ ID NO: 505, and the sequence of SEQ ID NO: 3649 or SEQ ID NO: 3451;
(ii) a second polypeptide chain comprising the sequence of SEQ ID NO: 3472, the sequence of SEQ ID NO: 2170, and the sequence of SEQ ID NO: 3648 or SEQ ID NO: 508 or 8686; and
(iii) a third polypeptide comprising the sequence of SEQ ID NO: 13 and the sequence of SEQ ID NO: 3644.

In some embodiments, the agent comprises:
(i) a first polypeptide chain comprising, from the N-terminus to the C-terminus, the sequence of SEQ ID NO: 505 operatively linked to the sequence of SEQ ID NO: 3649 or SEQ ID NO: 3451;
(ii) a second polypeptide chain comprising, from the N-terminus to the C-terminus, the sequence of SEQ ID NO: 3472 operatively linked to the sequence of SEQ ID NO: 2170 operatively linked to the sequence of SEQ ID NO: 3648 or SEQ ID NO: 508 or 8686; and
(iii) a third polypeptide comprising, from the N-terminus to the C-terminus, the sequence of SEQ ID NO: 13 operatively linked to the sequence of SEQ ID NO: 3644.

In some embodiments, the second polypeptide chain comprises, from the N-terminus to the C-terminus, the sequence of SEQ ID NO: 3472 operatively linked to the sequence of SEQ ID NO: 3473 operatively linked to the sequence of SEQ ID NO: 2170 operatively linked to the sequence of SEQ ID NO: 3308 operatively linked to the sequence of SEQ ID NO: 3648 or SEQ ID NO: 508 or 8686.

In some embodiments, the agent comprises:
(i) a first polypeptide chain comprising the sequence of SEQ ID NO: 505, and the sequence of SEQ ID NO: 3649 or SEQ ID NO: 3451;
(ii) a second polypeptide chain comprising the sequence of SEQ ID NO: 3474, and the sequence of SEQ ID NO: 3648 or SEQ ID NO: 508 or 8686; and
(iii) a third polypeptide comprising the sequence of SEQ ID NO: 13 and the sequence of SEQ ID NO: 3644.

In some embodiments, the agent comprises:
(i) a first polypeptide chain comprising, from the N-terminus to the C-terminus, the sequence of SEQ ID NO: 505 operatively linked to the sequence of SEQ ID NO: 3649 or SEQ ID NO: 3451;
(ii) a second polypeptide chain comprising, from the N-terminus to the C-terminus, the sequence of SEQ ID NO: 3474 operatively linked to the sequence of SEQ ID NO: 3648 or SEQ ID NO: 508 or 8686; and
(iii) a third polypeptide comprising, from the N-terminus to the C-terminus, the sequence of SEQ ID NO: 13 operatively linked to the sequence of SEQ ID NO: 3644.

In some embodiments, the second polypeptide comprises, from the N-terminus to the C-terminus, the sequence of SEQ ID NO: 3474 operatively linked to the sequence of SEQ ID NO: 3308 operatively linked to the sequence of SEQ ID NO: 3648 or SEQ ID NO: 508 or 8686.

In some embodiments, the agent comprises:
(i) a first polypeptide chain comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 500;
(ii) a second polypeptide chain comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 3420; and
(iii) a third polypeptide comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 501.

In some embodiments, the agent comprises:
(i) a first polypeptide chain comprising the sequence of SEQ ID NO: 500;
(ii) a second polypeptide chain comprising the sequence of SEQ ID NO: 3420; and
(iii) a third polypeptide comprising the sequence of SEQ ID NO: 501.

In some embodiments, the agent comprises:
(i) a first polypeptide chain comprising the sequence of SEQ ID NO: 505, and the sequence of SEQ ID NO: 3649 or SEQ ID NO: 3451;
(ii) a second polypeptide chain comprising the sequence of SEQ ID NO: 3472, the sequence of SEQ ID NO: 3670, and the sequence of SEQ ID NO: 3648 or SEQ ID NO: 508 or 8686; and
(iii) a third polypeptide comprising the sequence of SEQ ID NO: 13 and the sequence of SEQ ID NO: 3644.

In some embodiments, the agent comprises:
(i) a first polypeptide chain comprising, from the N-terminus to the C-terminus, the sequence of SEQ ID NO: 505 operatively linked to the sequence of SEQ ID NO: 3649 or SEQ ID NO: 3451;
(ii) a second polypeptide chain comprising, from the N-terminus to the C-terminus, the sequence of SEQ ID NO: 3472 operatively linked to the sequence of SEQ ID NO: 3670 operatively linked to the sequence of SEQ ID NO: 3648 or SEQ ID NO: 508 or 8686; and
(iii) a third polypeptide comprising, from the N-terminus to the C-terminus, the sequence of SEQ ID NO: 13 operatively linked to the sequence of SEQ ID NO: 3644.

In some embodiments, the second polypeptide comprises, from the N-terminus to the C-terminus, the sequence of SEQ ID NO: 3472 operatively linked to the sequence of SEQ ID NO: 3473 operatively linked to the sequence of SEQ ID NO: 3670 operatively linked to the sequence of SEQ ID NO: 3308 operatively linked to the sequence of SEQ ID NO: 3648 or SEQ ID NO: 508 or 8686.

In some embodiments, the agent comprises:
(i) a first polypeptide chain comprising the sequence of SEQ ID NO: 505, and the sequence of SEQ ID NO: 3649 or SEQ ID NO: 3451;
(ii) a second polypeptide chain comprising the sequence of SEQ ID NO: 3671, and the sequence of SEQ ID NO: 3648 or SEQ ID NO: 508 or 8686; and
(iii) a third polypeptide comprising the sequence of SEQ ID NO: 13 and the sequence of SEQ ID NO: 3644.

In some embodiments, the agent comprises:
(i) a first polypeptide chain comprising, from the N-terminus to the C-terminus the sequence of SEQ ID NO: 505 operatively linked to the sequence of SEQ ID NO: 3649 or SEQ ID NO: 3451;
(ii) a second polypeptide chain comprising, from the N-terminus to the C-terminus, the sequence of SEQ ID NO: 3671 operatively linked to the sequence of SEQ ID NO: 3648 or SEQ ID NO: 508 or 8686;
(iii) a third polypeptide comprising, from the N-terminus to the C-terminus, comprising the sequence of SEQ ID NO: 13, and the sequence of SEQ ID NO: 3644.

In some embodiments, the second polypeptide comprises, from the N-terminus to the C-terminus, the sequence of SEQ ID NO: 3671 operatively linked to the sequence of SEQ ID NO: 3308 operatively linked to the sequence of SEQ ID NO: 3648 or SEQ ID NO: 508 or 8686.

In some embodiments, the wherein the agent comprises:
(i) a first polypeptide chain comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 500;
(ii) a second polypeptide chain comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 3669; and
(iii) a third polypeptide comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 501.

In some embodiments, the wherein the agent comprises:
(i) a first polypeptide chain comprising the sequence of SEQ ID NO: 500;
(ii) a second polypeptide chain comprising the sequence of SEQ ID NO: 3669; and
(iii) a third polypeptide comprising the sequence of SEQ ID NO: 501.

In some embodiments, the agent comprises:
(i) a first polypeptide chain comprising the sequence of SEQ ID NO: 505, and the sequence of SEQ ID NO: 3649 or SEQ ID NO: 3451;
(ii) a second polypeptide chain comprising the sequence of 2270, and the sequence of SEQ ID NO: 3648 or SEQ ID NO: 508 or 8686; and
(iii) a third polypeptide comprising the sequence of SEQ ID NO: 13 and the sequence of SEQ ID NO: 3644.

In some embodiments, the agent comprises:
(i) a first polypeptide chain comprising, from the N-terminus to the C-terminus, the sequence of SEQ ID NO: 505 operatively linked to the sequence of SEQ ID NO: 3649 or SEQ ID NO: 3451;
(ii) a second polypeptide chain comprising, from the N-terminus to the C-terminus, the sequence of 2270 operatively linked to the sequence of SEQ ID NO: 3648 or SEQ ID NO: 508 or 8686; and
(iii) a third polypeptide comprising the sequence of SEQ ID NO: 13 and the sequence of SEQ ID NO: 3644.

In some embodiments, the second polypeptide comprises from the N-terminus to the C-terminus, the sequence of SEQ ID NO: 2270 operatively linked to the sequence of SEQ ID NO: 3308 operatively linked to the sequence of SEQ ID NO: 3648 or SEQ ID NO: 508 or 8686.

In some embodiments, the agent comprises:
(i) a first polypeptide chain comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 500;
(ii) a second polypeptide chain comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 3340; and
(iii) a third polypeptide comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 501.

In some embodiments, the agent comprises:
(i) a first polypeptide chain comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 4702;
(ii) a second polypeptide chain comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 4706; and
(iii) a third polypeptide comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 501.

In some embodiments, the agent comprises:
(i) a first polypeptide chain comprising the sequence of SEQ ID NO: 500;
(ii) a second polypeptide chain comprising the sequence of SEQ ID NO: 3340; and
(iii) a third polypeptide comprising the sequence of SEQ ID NO: 501.

In some embodiments, the agent comprises:
(i) a first polypeptide chain comprising the sequence of SEQ ID NO: 4702;
(ii) a second polypeptide chain comprising the sequence of SEQ ID NO: 3340; and
(iii) a third polypeptide comprising the sequence of SEQ ID NO: 4706.

In some embodiments, the agent comprises a first polypeptide and a second polypeptide, wherein the first polypeptide and the second polypeptide are non-contiguous, and wherein:
(i) the first polypeptide comprises a first member of the dimerization module linked to:
(a) the antigen binding domain comprising a VH of the antigen binding domain and a VL of the antigen binding domain, or
(b) a first portion of the antigen binding domain comprising the VH of the antigen binding domain, wherein the agent further comprises a third polypeptide comprising a second portion of the antigen binding domain comprising a VL of the antigen binding domain, wherein the third polypeptide is non-contiguous with the first polypeptide and the second polypeptide; and
(ii) the second polypeptide comprises a second member of the dimerization module linked to:
(a) an antigen binding domain that binds to T cell receptor beta chain constant domain (TRBC) comprising a VH and a VL, or
(b) a first portion of the antigen binding domain that binds to TRBC comprising the VH of the antigen binding domain that binds to TRBC, wherein the agent further comprises a fourth polypeptide comprising a second portion of the antigen binding domain that binds to TRBC comprising a VL of the antigen binding domain that binds to TRBC, wherein the fourth polypeptide is non-contiguous with the first polypeptide, the second polypeptide, and the third polypeptide.

In some embodiments, the agent comprises a first polypeptide and a second polypeptide, wherein the first polypeptide and the second polypeptide are non-contiguous, wherein:
(i) the first polypeptide comprises a first member of the dimerization module linked to a first portion of the antigen binding domain comprising the VH of the antigen binding domain, wherein the agent further comprises a third polypeptide comprising a second portion of the antigen binding domain comprising a VL of the antigen binding domain, wherein the third polypeptide is non-contiguous with the first polypeptide and the second polypeptide; and
(ii) the second polypeptide comprises a second member of the dimerization module linked to an antigen binding domain that binds to TRBC.

In some embodiments, the second antigen domain that binds to TRBC1 comprises a VH comprising:
(a) a VHCDR1 comprising the sequence of SEQ ID NO: 8643;
(b) a VHCDR2 comprising the sequence of SEQ ID NO: 7355 or SEQ ID NO: 201; and
(c) a VHCDR3 comprising the sequence of SEQ ID NO: 202.

In some embodiments, the second antigen domain that binds to TRBC2 comprises a VH comprising:
(a) a VHCDR1 comprising the sequence of SEQ ID NO: 8543, SEQ ID NO: 7401, or SEQ ID NO: 8272;
(b) a VHCDR2 comprising the sequence of SEQ ID NO: 201, SEQ ID NO: 8260, or SEQ ID NO: 8044; and
(c) a VHCDR3 comprising the sequence of SEQ ID NO: 7403, SEQ ID NO: 8227, SEQ ID NO: 8046, or SEQ ID NO: 8047.

In some embodiments, the second antigen binding domain that binds to TRBC1 comprises a VL comprising:
(a) a VLCDR1 comprising the sequence of SEQ ID NO: 223;
(b) a VLCDR2 comprising the sequence of SEQ ID NO: 224; and
(c) a VLCDR3 comprising the sequence of SEQ ID NO: 225 or 8680.

In some embodiments, the second antigen domain that binds to TRBC2 comprises a VL comprising:
(a) a VLCDR1 comprising the sequence of SEQ ID NO: 7410, SEQ ID NO: 8048, SEQ ID NO: 8051;
(b) a VLCDR2 comprising the sequence of SEQ ID NO: 224; and
(c) a VLCDR3 comprising the sequence of SEQ ID NO: 225 or 8680 or SEQ ID NO: 8052.

In some embodiments, the second antigen binding domain that binds to TRBC1 comprises:
(a) a VH comprising VHCDR1-3 that comprise the sequences of:
SEQ ID NOs: 8643, 7355, 202, respectively; or
SEQ ID NOs: 8643, 201, 202, respectively;
(b) a VL comprising VLCDR1-3 that comprise the sequences of:
SEQ ID NOs: 223, 224, 225 or 8680, respectively; or
(c) any combination thereof.

In some embodiments, the second antigen domain that binds to TRBC2 comprises:
(a) a VH comprising VHCDR1-3 that comprise the sequences of:
SEQ ID NOs: 8543, 201, 7403, respectively;
SEQ ID NOs: 7401, 201, 7403, respectively;
SEQ ID NOs: 8272, 8260, 8227, respectively;
SEQ ID NOs: 8272, 8044, 7403, respectively;
SEQ ID NOs: 8272, 8044, 8046, respectively; or
SEQ ID NOs: 8272, 8044, 8047, respectively;
(b) a VL comprising VLCDR1-3 that comprise the sequences of:
SEQ ID NOs: 7410, 224, 225 or 8680, respectively;
SEQ ID NOs: 8048, 224, 8050, respectively; or
SEQ ID NOs: 8051, 224, 8052, respectively; or
(c) any combination thereof.

In some embodiments, the second antigen domain that binds to TRBC1 comprises a VH comprising a sequence having at least 75% sequence identity to the sequence of SEQ ID NO: 517 or SEQ ID NO: 512.

In some embodiments, the second antigen domain that binds to TRBC2 comprises a VH comprising a sequence having at least 75% sequence identity to the sequence of SEQ ID NO: 7420, SEQ ID NO: 7423, SEQ ID NO: 8011, SEQ ID NO: 8013, SEQ ID NO: 8299, or SEQ ID NO: 8023.

In some embodiments, the second antigen domain that binds to TRBC1 comprises a VH comprising the sequence of SEQ ID NO: 517 or SEQ ID NO: 512.

In some embodiments, the second antigen domain that binds to TRBC2 comprises a VH comprising the sequence of SEQ ID NO: 7420, SEQ ID NO: 7423, SEQ ID NO: 8011, SEQ ID NO: 8013, SEQ ID NO: 8299, or SEQ ID NO: 8023.

In some embodiments, the second antigen domain that binds to TRBC1 comprises a VL comprising a sequence having at least 75% sequence identity to the sequence of SEQ ID NO: 8308.

In some embodiments, the second antigen domain that binds to TRBC2 comprises a VL comprising a sequence having at least 75% sequence identity to the sequence of SEQ ID NO: 7419, 8012, or 8014.

In some embodiments, the second antigen domain that binds to TRBC1 comprises a VL comprising the sequence of SEQ ID NO: 8308.

In some embodiments, the second antigen domain that binds to TRBC2 comprises a VL the sequence of SEQ ID NO: 7419, 8012, or 8014.

In some embodiments, the second antigen domain that binds to TRBC1 comprises: (a) a VH comprising a sequence having at least 75% sequence identity to the sequence of SEQ ID NO: 517 and a VL comprising a sequence having at least 75% sequence identity to the sequence SEQ ID NO: 8308; or (b) a VH comprising a sequence having at least 75% sequence identity to the sequence of SEQ ID NO: 512 and a VL comprising a sequence having at least 75% sequence identity to the sequence SEQ ID NO: 8308.

In some embodiments, the second antigen domain that binds to TRBC2 comprises:
(a) a VH comprising a sequence having at least 75% sequence identity to the sequence of SEQ ID NO: 7420 and a VL comprising a sequence having at least 75% sequence identity to the sequence of SEQ ID NO: 7419;
(b) a VH comprising a sequence having at least 75% sequence identity to the sequence of SEQ ID NO: 7423 and a VL comprising a sequence having at least 75% sequence identity to the sequence of SEQ ID NO: 7419;
(c) a VH comprising a sequence having at least 75% sequence identity to the sequence of SEQ ID NO: 8011 and a VL comprising a sequence having at least 75% sequence identity to the sequence of SEQ ID NO: 8012;
(d) a VH comprising a sequence having at least 75% sequence identity to the sequence of SEQ ID NO: 8013 and a VL comprising a sequence having at least 75% sequence identity to the sequence of SEQ ID NO: 8014;
(e) a VH comprising a sequence having at least 75% sequence identity to the sequence of SEQ ID NO: 8299 and a VL comprising a sequence having at least 75% sequence identity to the sequence of SEQ ID NO: 8014; or
(f) a VH comprising a sequence having at least 75% sequence identity to the sequence of SEQ ID NO: 8023 and a VL comprising a sequence having at least 75% sequence identity to the sequence of SEQ ID NO: 8014.

In some embodiments, the second antigen domain that binds to TRBC1 comprises:
(a) a VH comprising the sequence of SEQ ID NO: 517 and a VL comprising the sequence SEQ ID NO: 8308; or
(b) a VH comprising the sequence of SEQ ID NO: 512 and a VL comprising the sequence SEQ ID NO: 8308.

In some embodiments, the second antigen domain that binds to TRBC2 comprises:
(a) a VH comprising the sequence of SEQ ID NO: 7420 and a VL comprising the sequence of SEQ ID NO: 7419;
(b) a VH comprising the sequence of SEQ ID NO: 7423 and a VL comprising the sequence of SEQ ID NO: 7419;
(c) a VH comprising the sequence of SEQ ID NO: 8011 and a VL comprising the sequence of SEQ ID NO: 8012;
(d) a VH comprising the sequence of SEQ ID NO: 8013 and a VL comprising the sequence of SEQ ID NO: 8014;
(e) a VH comprising the sequence of SEQ ID NO: 8299 and a VL comprising the sequence of SEQ ID NO: 8014; or
(f) a VH comprising the sequence of SEQ ID NO: 8023 and a VL comprising the sequence of SEQ ID NO: 8014.

In some embodiments, the agent comprises:
(i) a first polypeptide chain comprising the sequence of SEQ ID NO: 505, the sequence of SEQ ID NO: 13 and the sequence of SEQ ID NO: 3447 or SEQ ID NO: 511;
(ii) a third polypeptide comprising the sequence of SEQ ID NO: 8308 and the sequence of SEQ ID NO: 3644; and
(iii) a second polypeptide chain comprising the sequence of SEQ ID NO: 517 and the sequence of SEQ ID NO: 513 or SEQ ID NO: 514; or a second polypeptide chain comprising the sequence of SEQ ID NO: 512 and the sequence of SEQ ID NO: 513 or SEQ ID NO: 514.

In some embodiments, the agent comprises:
(i) a first polypeptide chain comprising, from the N-terminus to the C-terminus, the sequence of SEQ ID NO: 505 operatively linked to the sequence of SEQ ID NO: 13 operatively linked to the sequence of SEQ ID NO: 3447 or SEQ ID NO: 511;
(ii) a third polypeptide comprising, from the N-terminus to the C-terminus, the sequence of SEQ ID NO: 8308 operatively linked to the sequence of SEQ ID NO: 3644; and
(iii) a second polypeptide chain comprising, from the N-terminus to the C-terminus, the sequence of SEQ ID NO: 517 operatively linked to the sequence of SEQ ID NO: 513 or SEQ ID NO: 514; or a second polypeptide chain comprising, from the N-terminus to the C-terminus, the sequence of SEQ ID NO: 512 operatively linked to the sequence of SEQ ID NO: 513 or SEQ ID NO: 514.

In some embodiments, the first polypeptide comprises, from the N-terminus to the C-terminus, the sequence of SEQ ID NO: 505 operatively linked to the sequence of SEQ ID NO: 8291 operatively linked to the sequence of SEQ ID NO: 13.

In some embodiments, the agent comprises:
(i) a first polypeptide chain comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 509 or the sequence SEQ ID NO: 4704;
(ii) a third polypeptide comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 7380; and
(iii) a second polypeptide chain comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 7382,the sequence of SEQ ID NO: 4677, the sequence of SEQ ID NO: 7379, or the sequence of SEQ ID NO: 4675.

In some embodiments, the agent comprises:
(i) a first polypeptide chain comprising the sequence of SEQ ID NO: 509 or the sequence SEQ ID NO: 4704;
(ii) a third polypeptide comprising the sequence of SEQ ID NO: 7380; and
(iii) a second polypeptide chain comprising the sequence of SEQ ID NO: 7382,the sequence of SEQ ID NO: 4677, the sequence of SEQ ID NO: 7379, or the sequence of SEQ ID NO: 4675.

In some embodiments, the agent comprises a first polypeptide chain comprising the sequence of SEQ ID NO: 505, the sequence of SEQ ID NO: 13 and the sequence of SEQ ID NO: 3447 or SEQ ID NO: 511.

In some embodiments, the agent comprises a first polypeptide chain comprising, from the N-terminus to the C-terminus, the sequence of SEQ ID NO: 505 operatively linked to the sequence of SEQ ID NO: 13 operatively linked to sequence of SEQ ID NO: 3447 or SEQ ID NO: 511.

In some embodiments, the first polypeptide comprises, from the N-terminus to the C-terminus, the sequence of SEQ ID NO: 505 operatively linked to the sequence of SEQ ID NO: 8291 operatively linked to the sequence of SEQ ID NO: 13.

In some embodiments, the agent comprises a first polypeptide chain comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 509.

In some embodiments, the agent comprises a first polypeptide chain comprising the sequence of SEQ ID NO: 509.

In some embodiments, the agent comprises a first polypeptide chain comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 4704.

In some embodiments, the agent comprises a first polypeptide chain comprising the sequence of SEQ ID NO: 4704.

In some embodiments, the agent comprises:
(i) a third polypeptide comprising the sequence of SEQ ID NO: 7419 and the sequence of SEQ ID NO: 3644; and
(ii) a second polypeptide chain comprising the sequence of SEQ ID NO: 7420 and the sequence of SEQ ID NO: 513 or SEQ ID NO: 514 or a second polypeptide chain comprising the sequence of SEQ ID NO: 7423 and the sequence of SEQ ID NO: 513 or SEQ ID NO: 514.

In some embodiments, the agent comprises:
(i) a third polypeptide comprising, from the N-terminus to the C-terminus, the sequence of SEQ ID NO: 7419 operatively linked to the sequence of SEQ ID NO: 3644; and
(ii) a second polypeptide chain comprising, from the N-terminus to the C-terminus, the sequence of SEQ ID NO: 7420 operatively linked to the sequence of SEQ ID NO: 513 or SEQ ID NO: 514 or a second polypeptide chain comprising the sequence of SEQ ID NO: 7423 operatively linked to the sequence of SEQ ID NO: 513 or SEQ ID NO: 514.

In some embodiments, the agent comprises:
(i) a third polypeptide comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 7439; and
(ii) a second polypeptide chain comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 4680, the sequence of SEQ ID NO: 7438, the sequence of SEQ ID NO: 4681, or the sequence of SEQ ID NO: 7440.

In some embodiments, the agent comprises:
(i) a third polypeptide comprising the sequence of SEQ ID NO: 7439; and
(ii) a second polypeptide chain comprising the sequence of SEQ ID NO: 4680, the sequence of SEQ ID NO: 7438, the sequence of SEQ ID NO: 4681, or the sequence of SEQ ID NO: 7440.

In some embodiments, the agent comprises:
(i) a third polypeptide comprising the sequence of SEQ ID NO: 8012 and the sequence of SEQ ID NO: 3644; and
(ii) a second polypeptide chain comprising the sequence of SEQ ID NO: 8011 and the sequence of SEQ ID NO: 513 or SEQ ID NO: 514.

In some embodiments, the agent comprises:
(i) a third polypeptide comprising, from the N-terminus to the C-terminus, the sequence of SEQ ID NO: 8012 operatively linked to the sequence of SEQ ID NO: 3644; and
(ii) a second polypeptide chain comprising, from the N-terminus to the C-terminus, the sequence of SEQ ID NO: 8011 operatively linked to the sequence of SEQ ID NO: 513 or SEQ ID NO: 514.

In some embodiments, the agent comprises:
(i) a third polypeptide comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 8002; and
(ii) a second polypeptide chain comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 8001 or 8697.

In some embodiments, the agent comprises:
(i) a third polypeptide comprising the sequence of SEQ ID NO: 8002; and
(ii) a second polypeptide chain comprising the sequence of SEQ ID NO: 8001 or 8697.

In some embodiments, the agent comprises:
(i) a third polypeptide comprising the sequence of SEQ ID NO: 8014 and the sequence of SEQ ID NO: 3644; and
(ii) a second polypeptide chain comprising the sequence of SEQ ID NO: 8013 and the sequence of SEQ ID NO: 513 or SEQ ID NO: 514, a second polypeptide chain comprising the sequence of SEQ ID NO: 8299 and the sequence of SEQ ID NO: 513 or SEQ ID NO: 514, or a second polypeptide chain comprising the sequence of SEQ ID NO: 8303 and the sequence of SEQ ID NO: 513 or SEQ ID NO: 514.

In some embodiments, the agent comprises:
(i) a third polypeptide comprising, from the N-terminus to the C-terminus, the sequence of SEQ ID NO: 8014 operatively linked to the sequence of SEQ ID NO: 3644; and
(ii) a second polypeptide chain comprising, from the N-terminus to the C-terminus, the sequence of SEQ ID NO: 8013 operatively linked to the sequence of SEQ ID NO: 513 or SEQ ID NO: 514, a second polypeptide chain comprising the sequence of SEQ ID NO: 8299 operatively linked to the sequence of SEQ ID NO: 513 or SEQ ID NO: 514, or a second polypeptide chain comprising the sequence of SEQ ID NO: 8303 operatively linked to the sequence of SEQ ID NO: 513 or SEQ ID NO: 514.

In some embodiments, the agent comprises:
(i) a third polypeptide comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 8005; and
(ii) a second polypeptide chain comprising a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 8004, the sequence of SEQ ID NO: 4685, the sequence of SEQ ID NO: 8007, the sequence of SEQ ID NO: 4686, the sequence of SEQ ID NO: 4687, or the sequence of SEQ ID NO: 8009.

In some embodiments, the agent comprises:
(i) a third polypeptide comprising the sequence of SEQ ID NO: 8005; and
(ii) a second polypeptide chain comprising the sequence of SEQ ID NO: 8004, the sequence of SEQ ID NO: 4685, the sequence of SEQ ID NO: 8007, the sequence of SEQ ID NO: 4686, the sequence of SEQ ID NO: 4687, or the sequence of SEQ ID NO: 8009.

In some embodiments, the agent further comprises at least one cytokine molecule or a functional fragment or variant thereof, wherein the at least one cytokine molecule or a functional fragment or variant thereof is linked to the first member of the dimerization module, the second member of the dimerization module, the antigen binding domain, the antigen binding domain that binds to TRBC, or any combination thereof.

In some embodiments, the at least one cytokine molecule or a functional fragment or variant thereof comprises IL-2 or a functional fragment or variant thereof, IL-7 or a functional fragment or variant thereof, IL-12 or a functional fragment or variant thereof, IL-15 or a functional fragment or variant thereof, IL-18 or a functional fragment or variant thereof, IL-21 or a functional fragment or variant thereof, interferon gamma or a functional fragment or variant thereof, or any combination thereof.

In some embodiments, the at least one cytokine molecule or a functional fragment or variant thereof comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one sequence selected from the group consisting of the cytokine sequences listed in Tables 40, 42C, 43A, 43B, and 46-51.

In some embodiments, the at least one cytokine molecule or a functional fragment or variant thereof comprises any one sequence selected from the group consisting of the cytokine sequences listed in Tables 40, 42C, 43A, 43B, and 46-51.

In some embodiments, the at least one cytokine molecule or a functional fragment or variant thereof comprises IL-2 or a functional fragment or variant thereof.

In some embodiments, the IL-2 or a functional fragment or variant thereof comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 2191, the sequence of SEQ ID NO: 2270, or the sequence of SEQ ID NO: 2280.

In some embodiments, the IL-2 or a functional fragment or variant thereof comprises a sequence the sequence of SEQ ID NO: 2191, the sequence of SEQ ID NO: 2270, or the sequence of SEQ ID NO: 2280.

In some embodiments, the at least one cytokine molecule or a functional fragment or variant thereof IL-15 or a functional fragment or variant thereof.

In some embodiments, the IL-15 or a functional fragment or variant thereof comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 2170, SEQ ID NO: 3670, or SEQ ID NO: 3673.

In some embodiments, the IL-15 or a functional fragment or variant thereof comprises the sequence of SEQ ID NO: 2170, the sequence of SEQ ID NO: 3670, or the sequence of SEQ ID NO: 3673.

In some embodiments, the at least one cytokine molecule or a functional fragment or variant thereof further comprises an IL15Ralpha dimerizing domain covalently linked the IL-15 or a functional fragment or variant thereof.

In some embodiments, the IL15Ralpha dimerizing domain comprises an IL-15 receptor alpha sushi domain or a functional fragment or variant thereof.

In some embodiments, the IL15Ralpha dimerizing domain comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 3472.

In some embodiments, the IL15Ralpha dimerizing domain comprises the sequence of SEQ ID NO: 3472.

In some embodiments, the IL-15 or a functional fragment or variant thereof is covalently linked to the IL15Ralpha dimerizing domain via a linker.

In some embodiments, the IL-15 or a functional fragment or variant thereof is covalently linked to the IL15Ralpha dimerizing domain via a linker comprising the sequence of SEQ ID NO: 3473.

In some embodiments, the at least one cytokine molecule or a functional fragment or variant thereof comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 3474, SEQ ID NO: 3671, or SEQ ID NO: 8689.

In some embodiments, the at least one cytokine molecule or a functional fragment or variant thereof comprises the sequence of SEQ ID NO: 3474, the sequence of SEQ ID NO: 3671, or the sequence of SEQ ID NO: 8689.

In some embodiments, the at least one cytokine molecule or a functional fragment or variant thereof comprises IL-7 or a functional fragment or variant thereof.

In some embodiments, the IL-7 or the functional fragment or variant thereof comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 3540.

In some embodiments, the IL-7 or the functional fragment or variant thereof comprises the sequence of SEQ ID NO: 3540.

In some embodiments, the at least one cytokine molecule or a functional fragment or variant thereof comprises IL-12 or a functional fragment or variant or variant thereof.

In some embodiments, the IL-12 or the functional fragment or variant thereof comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 3542, the sequence of SEQ ID NO: 3543, or the sequence of SEQ ID NO: 3545.

In some embodiments, the IL-12 or the functional fragment or variant thereof comprises the sequence of SEQ ID NO: 3542, the sequence of SEQ ID NO: 3543, or the sequence of SEQ ID NO: 3545.

In some embodiments, the at least one cytokine molecule or a functional fragment or variant thereof comprises IL-18 of a functional fragment or variant thereof.

In some embodiments, the IL-18 or the functional fragment or variant thereof comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 2192.

In some embodiments, the IL-18 or the functional fragment or variant thereof comprises the sequence of SEQ ID NO: 2192.

In some embodiments, the at least one cytokine molecule or a functional fragment or variant thereof comprises IL-21 or a functional fragment or variant thereof.

In some embodiments, the IL-21 or the functional fragment or variant thereof comprises a sequence having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to the sequence of SEQ ID NO: 3540.

In some embodiments, the IL-21 or the functional fragment or variant thereof comprises the sequence of SEQ ID NO: 3540.

In another aspect, provided herein is a recombinant polynucleotide molecule comprising a polynucleotide sequence encoding the agent of the composition as provided herein.

In some embodiments, the recombinant polynucleotide molecule is an isolated recombinant polynucleotide molecule.

In another aspect, provided herein is a vector comprising the recombinant polynucleotide molecule as provided herein.

In another aspect, provided herein is a cell comprising the recombinant polynucleotide molecule as provided herein, or the vector as provided herein.

In another aspect, provided herein is a method of making the agent of the composition of any one as provided herein, comprising culturing the cell as provided herein under conditions suitable for expression of the agent.

In another aspect, provided herein is a pharmaceutical composition comprising the composition as provided herein, the recombinant polynucleotide molecule as provided herein, the vector as provided herein, or the cell as provided herein, and a pharmaceutically acceptable carrier, excipient, or diluent.

In another aspect, provided herein is a method of treating a condition or disease in a subject in need thereof comprising administering to the subject a therapeutically effective amount of the composition as provided herein, the recombinant polynucleotide molecule as provided herein, the vector as provided herein, the cell as provided herein, the pharmaceutical composition as provided herein, or any combination thereof, thereby treating the condition or disease in the subject.

In another aspect, provided herein is the use of the composition as provided herein, the recombinant polynucleotide molecule as provided herein, the vector as provided herein, the cell as provided herein, the pharmaceutical composition as provided herein, or any combination thereof for a method of treating a condition or disease in a subject in need thereof, wherein the method comprises administering to the subject a therapeutically effective amount of the composition, the recombinant polynucleotide molecule, the vector, the cell as provided herein, the pharmaceutical composition as provided herein, or any combination thereof, thereby treating the condition or disease in the subject.

In another aspect, provided herein is the use of the composition as provided herein, the recombinant polynucleotide molecule as provided herein, the vector as provided herein, the cell as provided herein, the pharmaceutical composition as provided herein, or any combination thereof for treating a condition or disease in a subject in need thereof, wherein the composition, the recombinant polynucleotide molecule, the vector, the cell as provided herein, the pharmaceutical composition as provided herein, or any combination thereof is formulated for administration to the subject in a therapeutically effective amount.

In some embodiments, the therapeutically effective amount is an amount sufficient to treat the condition or disease in the subject.

In another aspect, provided herein is the composition as provided herein, the recombinant polynucleotide molecule as provided herein, the vector as provided herein, the cell as provided herein, the pharmaceutical composition as provided herein, or any combination thereof for use as a medicament.

In another aspect, provided herein is the composition as provided herein, the recombinant polynucleotide molecule as provided herein, the vector as provided herein, the cell as provided herein, the pharmaceutical composition as provided herein, or any combination thereof for use in the manufacture of a medicament for treating a condition or disease in a subject in need thereof.

In some embodiments, the composition as provided herein, the recombinant polynucleotide molecule as provided herein, the vector as provided herein, the cell as provided herein, the pharmaceutical composition as provided herein, or any combination thereof is formulated for administration to the subject in a therapeutically effective amount.

In some embodiments, the therapeutically effective amount is an amount sufficient to treat the condition or disease in the subject.

In some embodiments, the condition or disease is cancer.

In some embodiments, the cancer is leukemia or lymphoma.

In some embodiments, the cancer is selected from the group consisting of acquired immune deficiency syndrome (AIDS)-associated lymphoma, Angioimmunoblastic T-cell lymphoma, Adult T-cell leukemia/lymphoma, Burkitt lymphoma, Central nervous system (CNS) lymphoma, Diffuse large B-cell lymphoma (DLBCL), Lymphoblastic lymphoma, Mantle cell lymphoma (MCL), Peripheral T-cell lymphoma (PTCL), Transformed follicular and transformed mucosa-associated lymphoid tissue (MALT) lymphomas, Cutaneous T-cell lymphoma (mycosis fungoides and Sézary syndrome), Follicular lymphoma, Lymphoplasmacytic lymphoma/Waldenström macroglobulinemia, Marginal zone B-cell lymphoma, Gastric mucosa-associated lymphoid tissue (MALT) lymphoma, Chronic lymphocytic leukemia/small-cell lymphocytic lymphoma (CLL/SLL), Extranodal T-/NK-cell lymphoma, and Anaplastic large-cell lymphoma.

In some embodiments, peripheral T-cell lymphoma (PTCL) is Hepatosplenic T-cell lymphoma (HSGDTCL), Subcutaneous paniculitis-like T-cell lymphoma, or Enteropathy-associated T-cell lymphoma.

In some embodiments, the cutaneous T-cell lymphoma is mycosis fungoides and Sézary syndrome.

In some embodiments, the extranodal T-/NK-cell lymphoma is a nasal type.

In some embodiments, the anaplastic large-cell lymphoma is primary cutaneous anaplastic large-cell lymphoma or systemic anaplastic large-cell lymphoma.

In some embodiments, the cancer is a solid tumor.

In some embodiments, the solid tumor is selected from the group consisting of pancreatic cancer, breast cancer, colon cancer, colorectal cancer, lung cancer, skin cancer, ovarian cancer, liver cancer, esophageal cancer, endometrial cancer, gastric cancer, head and neck cancer, kidney cancer, and prostate cancer.

In some embodiments, the pancreatic cancer is pancreatic adenocarcinoma or pancreatic ductal adenocarcinoma.

In some embodiments, the lung cancer is small cell lung cancer (SCLC) or non-small cell lung cancer (NSCLC).

In another aspect, provided herein is a method of expanding and/or activating NK cells in a NK cell population comprising contacting the NK cell population with an effective amount of the composition as provided herein, the recombinant polynucleotide molecule as provided herein, the vector as provided herein, the cell as provided herein, the pharmaceutical composition as provided herein, or any combination thereof, thereby, expanding and/or activating the NK cells in the NK cell population.

In another aspect, provided herein is the use of the composition as provided herein, the recombinant polynucleotide molecule as provided herein, the vector as provided herein, the cell as provided herein, the pharmaceutical composition as provided herein, or any combination thereof for a method of expanding and/or activating NK cells in a NK cell population, wherein the method comprises contacting the NK cell population with an effective amount of the composition, the recombinant polynucleotide molecule, the vector, the cell as provided herein, the pharmaceutical composition as provided herein, or any combination thereof, thereby expanding and/or activating the NK cells in the NK cell population.

In another aspect, provided herein is the use of the composition as provided herein, the recombinant polynucleotide molecule as provided herein, the vector as provided herein, the cell as provided herein, the pharmaceutical composition as provided herein, or any combination thereof for expanding and/or activating NK cells in a NK cell population, wherein the composition, the recombinant polynucleotide molecule, the vector, the cell as provided herein, the pharmaceutical composition as provided herein, or any combination thereof is formulated for contacting the NK cell population in an effective amount.

In another aspect, provided herein is the composition as provided herein, the recombinant polynucleotide molecule as provided herein, the vector as provided herein, the cell as provided herein, the pharmaceutical composition as provided herein, or any combination thereof for use in the manufacture of a medicament for expanding and/or activating NK cells in a NK cell population.

In some embodiments, the composition as provided herein, the recombinant polynucleotide molecule as provided herein, the vector as provided herein, the cell as provided herein, the pharmaceutical composition as provided herein, or any combination thereof is formulated for expanding and/or activating NK cells in a NK cell population in an effective amount.

In some embodiments, the expanding and/or activating NK cells is in vivo or ex vivo expansion and/or activation.

In some embodiments, the expanded and/or activated NK cells are administered to a subject in need thereof.

In some embodiments, the expanded and/or activated NK cells are autogenic to the subject.

In some embodiments, the expanded and/or activated NK cells are allogenic to the subject.

In some embodiments, the first polypeptide or the first polypeptide chain comprises one or more sequences having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one of sequences listed in the tables as provided herein.

In some embodiments, the second polypeptide or the second polypeptide chain comprises one or more sequences having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one of sequences listed in the tables as provided herein.

In some embodiments, the third polypeptide comprises one or more sequences having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one of sequences listed in the tables as provided herein.

In some embodiments, the fourth polypeptide comprises one or more sequences having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one of sequences listed in the tables as provided herein.

In some embodiments, the first polypeptide or the first polypeptide chain comprises one or more sequences listed in the tables as provided herein.

In some embodiments, the second polypeptide or the second polypeptide chain comprises one or more sequences listed in the tables as provided herein.

In some embodiments, the third polypeptide comprises one or more sequences listed in the tables as provided herein.

In some embodiments, the fourth polypeptide comprises one or more sequences listed in the tables as provided herein.

In some embodiments, the first polypeptide or the first polypeptide chain comprises one or more sequences having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one of sequences listed in Tables 1, 2A, 2B, 3A, 3B, 3, 4, 5A, 5B, 6-8, 9A, 9B, 10-18, 20A, 20B, 21A, 21B, 22, 23A, 23B, 24-27, 30, 32, 39, 40, 42A, 42B, 42C, 43A, 43B, 43-51.

In some embodiments, the second polypeptide or the second polypeptide chain comprises one or more sequences having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one of sequences listed in Tables 1, 2A, 2B, 3A, 3B, 3, 4, 5A, 5B, 6-8, 9A, 9B, 10-18, 20A, 20B, 21A, 21B, 22, 23A, 23B, 24-27, 30, 32, 39, 40, 42A, 42B, 42C, 43A, 43B, 43-51.

In some embodiments, the third polypeptide comprises one or more sequences having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one of sequences listed in Tables 1, 2A, 2B, 3A, 3B, 3, 4, 5A, 5B, 6-8, 9A, 9B, 10-18, 20A, 20B, 21A, 21B, 22, 23A, 23B, 24-27, 30, 32, 39, 40, 42A, 42B, 42C, 43A, 43B, 43-51.

In some embodiments, the fourth polypeptide comprises one or more sequences having at least 30%, 35%, 40%, 45%, 50%, 55%, 60%, 65%, 70%, 75%, 80%, 85%, 90%, 95%, 96%, 97%, 98%, 99%, 99.5%, 99.6%, 99.7%, 99.8%, 99.9%, or 100% sequence identity to any one of sequences listed in Tables 1, 2A, 2B, 3A, 3B, 3, 4, 5A, 5B, 6-8, 9A, 9B, 10-18, 20A, 20B, 21A, 21B, 22, 23A, 23B, 24-27, 30, 32, 39, 40, 42A, 42B, 42C, 43A, 43B, 43-51.

In some embodiments, the first polypeptide or the first polypeptide chain comprises one or more sequences listed in Tables 1, 2A, 2B, 3A, 3B, 3, 4, 5A, 5B, 6-8, 9A, 9B, 10-18, 20A, 20B, 21A, 21B, 22, 23A, 23B, 24-27, 30, 32, 39, 40, 42A, 42B, 42C, 43A, 43B, 43-51.

In some embodiments, the second polypeptide or the second polypeptide chain comprises one or more sequences listed in Tables 1, 2A, 2B, 3A, 3B, 3, 4, 5A, 5B, 6-8, 9A, 9B, 10-18, 20A, 20B, 21A, 21B, 22, 23A, 23B, 24-27, 30, 32, 39, 40, 42A, 42B, 42C, 43A, 43B, 43-51.

In some embodiments, the third polypeptide comprises one or more sequences listed in Tables 1, 2A, 2B, 3A, 3B, 3, 4, 5A, 5B, 6-8, 9A, 9B, 10-18, 20A, 20B, 21A, 21B, 22, 23A, 23B, 24-27, 30, 32, 39, 40, 42A, 42B, 42C, 43A, 43B, 43-51.

In some embodiments, the fourth polypeptide comprises one or more sequences listed in Tables 1, 2A, 2B, 3A, 3B, 3, 4, 5A, 5B, 6-8, 9A, 9B, 10-18, 20A, 20B, 21A, 21B, 22, 23A, 23B, 24-27, 30, 32, 39, 40, 42A, 42B, 42C, 43A, 43B, 43-51.

### Methods of Treatment

Described herein, in certain embodiments, is a method of treating a condition or disease in a subject in need therefor comprising administering to the subject a therapeutically effective amount of the antibody molecule, multifunctional molecule, or multispecific molecule as described herein, the nucleic acid molecules as described herein, the vector as described herein, the cell as described herein, the pharmaceutical composition as described herein, or a combination thereof, wherein the administering is effective to treat the condition or disease in the subject. Any condition or disease that is related to TCRαV and/or TCRβV may be subject of the methods of treatment disclosed herein. For example, a condition or disease wherein autoreactive T cells of a subject that expresses a TCRαV and/or a TCRβV can be treated with a CAR-T cell containing a CAR with an anti-TCRαV and/or anti-TCRβV binding domain that binds to the TCRαV and/or TCRβV expressed by autoreactive T cells. Examples of TCRαV- and TCRβV-related diseases include, but are not limited to, those listed in Table 52.

In some embodiments, the condition or disease is an autoimmune disease. In some embodiments, the autoimmune disease is selected from the group consisting of the autoimmune disease is selected from amyotrophic lateral sclerosis (ALS), coeliac disease (CD), ankylosing spondylitis (AS), Covid-induced multisystem inflammatory syndrome in children (MIS-C), primary Sjogren's syndrome (PSS), Churg-Strauss syndrome, sarcoidosis, systemic lupus erythematosus (SLE), type 1 diabetes, autoimmune hepatitis (e.g., type 1 or type 2), primary sclerosing cholangitis, primary biliary cirrhosis, multiple sclerosis, Guillain-Barre syndrome and the AMAN (axonal & neuronal neuropathy), chronic inflammatory demyelinating polyneuropathy (CIDP), transverse myelitis, Tolosa-Hunt syndrome (THS), Devic's disease (neuromyelitis optica), paraneoplastic cerebellardegeneration (PCD), Lambert-Eaton syndrome, psoriasis, scleroderma, CREST (calcinosis,Raynaud phenomenon, esophageal dysmotility, sclerodactyly, and telangiectasia) syndrome, dermatitis herpetiformis, dermatomyositis, bullous pemphigoid, cicatricial pemphigoid/benign mucosal pemphigoid, pemphigoid gestationis, rheumatoid arthritis (RA), psoriatic arthritis, relapsing polychondritis, chronic recurrent multifocal osteomyelitis (CRMO), vasculitis, Kawasaki disease, granulomatosis with polyangiitis (GPA), Behcet's disease (vasculitis),Takayasu's arteritis, polyarteritis nodosa, microscopic polyangiitis (MPA), leukocytoclasticvasculitis, Cogan's syndrome, uveitis, peripheral uveitis (Pars planitis), scleritis, autoimmune inner ear disease (AIED), Crohn's, ulcerative colitis (UC), Dressler's syndrome, Rheumatic fever, Evans syndrome, paroxysmal nocturnal hemoglobinuria (PNH), hemolytic anemia, thrombocytopenic purpura (TTP), polymyositis, juvenile myositis (JM), including Juvenile Dermatomyositis (JDM) and Juvenile Polymyositis (JPM), ocular cicatricial pemphigoid, or Hashimoto's thyroiditis.

In some embodiments, the molecules or the composition disclosed herein (e.g., antibody molecules or multispecific molecules (or pharmaceutical composition) provided herein, the nucleic acid molecules as described herein, the vector as described herein, the cell as described herein, the pharmaceutical composition as described herein, or a combination thereof), are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the molecules or the composition disclosed herein to be administered can be determined by a physician with consideration of individual differences in severity/character of immune disorder, extent of infection or metastasis, age, weight, and condition of the subject.

In some embodiments, binding of the molecules or the composition disclosed herein results in cell killing, e.g., target cell killing, e.g. autoreactive T cell killing. In some embodiments, the autoreactive T cell is a CD4+ T cell. In some embodiments, the autoreactive T cell is a CD8+ T cell. In some embodiments, binding of the molecules or the composition disclosed herein results in cell killing in vitro or in vivo.

In embodiments, the molecules or the composition disclosed herein is administered to the subject parenterally. In embodiments, the cells disclosed herein are administered to the subject intravenously, subcutaneously, intranodally, intramuscularly, intradermally, or intraperitoneally. In embodiments, the cells are administered as an infusion (e.g., as described in Rosenberg et al., New Eng. J. of Med. 319:1676, 1988) or an intravenous push. In embodiments, the cells are administered as an injectable depot formulation.

In embodiments, the subject is a mammal. In embodiments, the subject is a human, monkey, pig, dog, cat, cow, sheep, goat, rabbit, rat, or mouse. In embodiments, the subject is a human. In embodiments, the subject is a pediatric subject, e.g., less than 18 years of age, e.g., less than 17, 16, 15, 14, 13, 12, 11, 10, 9, 8, 7, 6, 5, 4, 3, 2, 1 or less years of age. In embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

Without wishing to be bound by any particular theory, the immune response elicited by the anti-TCRVα and/or anti-TCRβV TCR targeting molecules may be an active or a passive immune response. In one embodiment, the anti-TCRVα and/or anti-TCRβV TCR targeting molecules of the present disclosure may be a type of vaccine for ex vivo immunization and/or in vivo therapy in a mammal. With respect to ex vivo immunization, at least one of the following occurs in vitro prior to administering the cell into a mammal: i) expansion of the cells, ii) introducing a nucleic acid encoding an anti-TCRVα and/or anti-TCRβV TCR targeting molecules to the cells or iii) cryopreservation of the cells.

Ex vivo procedures are well known in the art and are discussed more fully below. Briefly, cells are isolated from a mammal (e.g., a human) and genetically modified (i.e., transduced or transfected in vitro) with a vector expressing an anti-TCRVα and/or anti-TCRβV TCR targeting molecule disclosed herein.

Methods described herein include treating an autoimmune disease in a subject by using the molecules or the composition disclosed herein, e.g., using a pharmaceutical composition described herein. Also provided are methods for reducing or ameliorating a symptom of an autoimmune disease in a subject. In some embodiments, the methods described herein re-balance a T cell repertoire in a subject (e.g. reduce or deplete autoreactive T cells and preserve healthy T cells to establish a T cell repertoire more similar to a person without an autoimmune disease).

Disclosed herein are methods of reducing a symptom of an autoimmune disease in a subject in need thereof, comprising administering to the subject a therapeutically effective amount of an agent comprising a moiety that binds to a TCR variable region (e.g. a TCRVα and/or TCRβV region) to the subject, thereby inhibiting or preventing the activation or expansion of autoreactive T cells in the subject. In some embodiments, the agent comprises an anti-TCRαV antibody comprising a variant Fc region. In some embodiments, the agent comprises an anti-TCRβV antibody comprising a variant Fc region. In some embodiments, the variant Fc region has increased binding to C1q complement. In some embodiments, the variant Fc region has increased effector function (e.g., increased antibody dependent cell-mediated cytotoxicity (ADCC), antibody-dependent cellular phagocytosis (ADCP) and/or complement dependent cytotoxicity (CDC).

Disclosed herein are methods of treating a disease or condition in a subject in need thereof, comprising administering to the subject a therapeutically effective amount of an agent comprising a moiety that binds to a TCR variable region (e.g. a TCRVα and/or TCRβV region) to the subject, thereby inhibiting or preventing the activation or expansion of autoreactive T cells in the subject. In some embodiments, the agent comprises an anti-TCRαV antibody comprising a variant Fc region. In some embodiments, the agent comprises an anti-TCRβV antibody comprising a variant Fc region. In some embodiments, the variant Fc region has increased binding to C1q complement. In some embodiments, the variant Fc region has increased effector function (e.g., increased antibody dependent cell-mediated cytotoxicity (ADCC), antibody-dependent cellular phagocytosis (ADCP) and/or complement dependent cytotoxicity (CDC). In some embodiments, the disease or condition is an autoimmune disease.

Disclosed herein are methods of reducing a symptom of an autoimmune disease in a subject in need thereof, comprising administering to the subject a therapeutically effective amount of an agent comprising a moiety that binds to a TCR variable region (e.g. a TCRVα and/or TCRβV region) to the subject. In some embodiments, the agent comprises an antibody-drug conjugate. In some embodiments, the antibody-drug conjugate comprises a TCRαV antigen binding domain. In some embodiments, the antibody-drug conjugate comprises a TCRβV antigen binding domain. In some embodiments, the antibody-drug conjugate has no mutation to an Fc region. In some embodiments, the antibody-drug conjugate is functionally linked to a cytotoxic agent. In some embodiments, the cytotoxic agent is an inhibitor of tubulin polymerization. In some embodiments, the cytotoxic agent is selected from the group consisting of maytansine, dolastatin, auristatin drug analogs, duocarmycin derivatives (e.g. CC-1065 analogs and duocarmycin), esperamicin, calicheamicin, and pyrrolobenzodiazepine (PBD).

Disclosed herein are methods of treating a disease or condition in a subject in need thereof, comprising administering to the subject a therapeutically effective amount of an agent comprising a moiety that binds to a TCR variable region (e.g. a TCRVα and/or TCRβV region) to the subject. In some embodiments, the agent comprises an antibody-drug conjugate. In some embodiments, the antibody-drug conjugate comprises a TCRαV antigen binding domain. In some embodiments, the antibody-drug conjugate comprises a TCRβV antigen binding domain. In some embodiments, the antibody-drug conjugate has no mutation to an Fc region. In some embodiments, the antibody-drug conjugate is functionally linked to a cytotoxic agent. In some embodiments, the cytotoxic agent is an inhibitor of tubulin polymerization. In some embodiments, the cytotoxic agent is selected from the group consisting of maytansine, dolastatin, auristatin drug analogs, duocarmycin derivatives (e.g. CC-1065 analogs and duocarmycin), esperamicin, calicheamicin, and pyrrolobenzodiazepine (PBD). In some embodiments, the disease or condition is an autoimmune disease.

Disclosed herein are methods of reducing a symptom of an autoimmune disease in a subject in need thereof, comprising administering to the subject a therapeutically effective amount of an agent comprising a moiety that binds to a TCR variable region (e.g. a TCRVα and/or TCRβV region) to the subject. In some embodiments, the agent is a CAR T cell comprising a CAR with an anti-TCRαV binding domain that binds to the TCRαV expressed by the autoreactive T cells. In some embodiments, the agent is a CAR T cell comprising a CAR with an anti-TCRβV binding domain that binds to the TCRβV expressed by the autoreactive T cells. In some embodiments, the CAR comprises a transmembrane domain as provided herein. In some embodiments, the CAR comprises an intracellular domain as provided herein.

Disclosed herein are methods of treating a disease or condition in a subject in need thereof, comprising administering to the subject a therapeutically effective amount of an agent comprising a moiety that binds to a TCR variable region (e.g. a TCRVα and/or TCRβV region) to the subject. In some embodiments, the agent is a CAR T cell comprising a CAR with an anti-TCRαV binding domain that binds to the TCRαV expressed by the autoreactive T cells. In some embodiments, the agent is a CAR T cell comprising a CAR with an anti-TCRβV binding domain that binds to the TCRβV expressed by the autoreactive T cells. In some embodiments, the CAR comprises a transmembrane domain as provided herein. In some embodiments, the CAR comprises an intracellular domain as provided herein. In some embodiments, the disease or condition is an autoimmune disease.

Disclosed herein are methods of reducing a symptom of an autoimmune disease in a subject in need thereof, comprising administering to the subject a therapeutically effective amount of an agent comprising a moiety that binds to a TCR variable region (e.g. a TCRVα and/or TCRβV region) to the subject. In some embodiments, the subject is administered a multispecific molecule comprising a TCRVα binding region and a NK cell engager. In some embodiments, the NK cell engager is selected from an antigen binding domain or ligand that binds to (e.g., activates): NKp30, NKp40, NKp44, NKp46, NKG2D, DNAM1, DAP10, CD16 (e.g., CD16a, CD16b, or both), CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS1, KIR2DS3, KIR2DS5, KIR2DS1, CD94, NKG2C, NKG2E, or CD160. In some embodiments, the NK cell engager is a ligand of CD16, which is a CD16a/b ligand, e.g., a CD16a/b ligand further comprising an antibody Fc region. In some embodiments, the NK cell engager is an agonist for a NK activating receptor. In some embodiments, the NK cell engager is an antagonist for a NK inhibitory receptor. In some embodiments, the multispecific molecule has no Fc region mutation.

Disclosed herein are methods of treating a disease or condition in a subject in need thereof, comprising administering to the subject a therapeutically effective amount of an agent comprising a moiety that binds to a TCR variable region (e.g. a TCRVα and/or TCRβV region) to the subject. In some embodiments, the subject is administered a multispecific molecule comprising a TCRVα binding region and a NK cell engager. In some embodiments, the NK cell engager is selected from an antigen binding domain or ligand that binds to (e.g., activates): NKp30, NKp40, NKp44, NKp46, NKG2D, DNAM1, DAP10, CD16 (e.g., CD16a, CD16b, or both), CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS1, KIR2DS3, KIR2DS5, KIR2DS1, CD94, NKG2C, NKG2E, or CD160. In some embodiments, the NK cell engager is a ligand of CD16, which is a CD16a/b ligand, e.g., a CD16a/b ligand further comprising an antibody Fc region. In some embodiments, the NK cell engager is an agonist for a NK activating receptor. In some embodiments, the NK cell engager is an antagonist for a NK inhibitory receptor. In some embodiments, the multispecific molecule has no Fc region mutation. In some embodiments, the subject has an autoimmune disease.

Disclosed herein are methods of reducing a symptom of an autoimmune disease in a subject in need thereof, comprising administering to the subject a therapeutically effective amount of an agent comprising a moiety that binds to a TCR variable region (e.g. a TCRVα and/or TCRβV region) to the subject. In some embodiments, the subject is administered an agent comprising a multispecific molecule comprising an antigen binding domain that binds to TCRVα and a T cell engager. In some embodiments, the subject is administered an agent comprising a multispecific molecule comprising an antigen binding domain that binds to TCRVβ and a T cell engager. In some embodiments, the T cell engager is selected from an antigen binding domain or ligand that binds to (e.g., and in some embodiments activates) one or more of CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, or CD226. In some embodiments, the T cell engager binds to (e.g. activates) a CD4+ and/or a CD8+ T cell. In some embodiments, the T cell engager binds to (e.g. activates) a CD4+ T cell. In some embodiments, the T cell engager binds to (e.g. activates) a CD8+ T cell. In some embodiments, the disease or condition is an autoimmune disease.

In some aspects, the present disclosure provides a method of treating a disease or condition. The method provided herein may also comprise reducing a risk of developing the disease or condition in a subject in need thereof. The method may comprise administering to the subject a composition. The composition can comprise an agent. The agent can comprise a moiety that binds to a TCR variable region. In some embodiments, the moiety can bind to a T cell receptor alpha variable region (TCRαV) or a T cell receptor variable beta region (TCRβV). In some embodiments, the disease or condition can be multiple sclerosis, and the moiety that binds to the TCR variable region may bind to a TCRβ V6 subfamily; the disease or condition can be acute anterior uveitis (UAA), and the moiety that binds to the TCR variable region may bind to a TCRβ V9 subfamily; the disease or condition can be psoriatic arthritis (PsA), and the moiety that binds to the TCR variable region may bind to a TCRβ V9 subfamily; the disease or condition can be type I narcolepsy, and the moiety that binds to the TCR variable region may bind to a TCRβ V4 subfamily; the disease or condition can be amyotrophic lateral sclerosis (ALS), and the moiety that binds to the TCR variable region may bind to a TCRα V13 subfamily; or the disease or condition can be UAA, and the moiety that binds to the TCR variable region may bind to a TCRα V21 subfamily.

As an example, the present disclosure provides a method treating a disease or condition or reducing a risk of developing the disease or condition in a subject in need thereof, comprising administering to the subject a composition, wherein the composition comprises an agent comprising a moiety that binds to a TCR variable region, wherein the TCR variable region is a T cell receptor alpha variable region (TCRαV) or a T cell receptor variable beta region (TCRβV), and wherein: the disease or condition is multiple sclerosis, and the moiety that binds to the TCR variable region binds to a TCRβ V6 subfamily; the disease or condition is acute anterior uveitis (UAA), and the moiety that binds to the TCR variable region binds to a TCRβ V9 subfamily; the disease or condition is psoriatic arthritis (PsA), and the moiety that binds to the TCR variable region binds to a TCRβ V9 subfamily; the disease or condition is type I narcolepsy, and the moiety that binds to the TCR variable region binds to a TCRβ V4 subfamily; the disease or condition is amyotrophic lateral sclerosis (ALS), and the moiety that binds to the TCR variable region binds to a TCRα V13 subfamily; or the disease or condition is UAA, and the moiety that binds to the TCR variable region binds to a TCRα V21 subfamily.

In some embodiments, the agent may be present in the composition at a concentration. In some embodiments, the agent may be present in the composition described herein at a concentration of at least about 0.0001 mg/ml, at least about 0.001 mg/ml, at least about 0.005 mg/ml, at least about 0.01 mg/ml, at least about 0.05 mg/ml, at least about 0.1 mg/ml, at least about 0.5 mg/ml, at least about 1.0 mg/ml, at least about 2.0 mg/ml, at least about 3.0 mg/ml, at least about 4.0 mg/ml, at least about 5.0 mg/ml, at least about 10.0 mg/ml, or greater than about 10.0 mg/ml. In some embodiments, the agent may be present in the composition described herein at a concentration of at most about 10.0 mg/ml, at most about 5.0 mg/ml, at most about 4.0 mg/ml, at most about 3.0 mg/ml, at most about 2.0 mg/ml, at most about 1.0 mg/ml, at most about 0.5 mg/ml, at most about 0.1 mg/ml, at most about 0.05 mg/ml, at most about 0.01 mg/ml, at most about 0.005 mg/ml, at most about 0.001 mg/ml, at most about 0.0001 mg/ml, or less than about 0.0001 mg/ml. In some embodiments, the agent may be present in the composition described herein at a concentration between about 0.0001 mg/ml to about 5 mg/ml. In some embodiments, the agent may be present in the composition described herein at a concentration between about 0.0001 mg/ml to about 0.001 mg/ml, about 0.0001 mg/ml to about 0.005 mg/ml, about 0.0001 mg/ml to about 0.01 mg/ml, about 0.0001 mg/ml to about 0.05 mg/ml, about 0.0001 mg/ml to about 0.1 mg/ml, about 0.0001 mg/ml to about 0.5 mg/ml, about 0.0001 mg/ml to about 1 mg/ml, about 0.0001 mg/ml to about 2 mg/ml, about 0.0001 mg/ml to about 3 mg/ml, about 0.0001 mg/ml to about 4 mg/ml, about 0.0001 mg/ml to about 5 mg/ml, about 0.001 mg/ml to about 0.005 mg/ml, about 0.001 mg/ml to about 0.01 mg/ml, about 0.001 mg/ml to about 0.05 mg/ml, about 0.001 mg/ml to about 0.1 mg/ml, about 0.001 mg/ml to about 0.5 mg/ml, about 0.001 mg/ml to about 1 mg/ml, about 0.001 mg/ml to about 2 mg/ml, about 0.001 mg/ml to about 3 mg/ml, about 0.001 mg/ml to about 4 mg/ml, about 0.001 mg/ml to about 5 mg/ml, about 0.005 mg/ml to about 0.01 mg/ml, about 0.005 mg/ml to about 0.05 mg/ml, about 0.005 mg/ml to about 0.1 mg/ml, about 0.005 mg/ml to about 0.5 mg/ml, about 0.005 mg/ml to about 1 mg/ml, about 0.005 mg/ml to about 2 mg/ml, about 0.005 mg/ml to about 3 mg/ml, about 0.005 mg/ml to about 4 mg/ml, about 0.005 mg/ml to about 5 mg/ml, about 0.01 mg/ml to about 0.05 mg/ml, about 0.01 mg/ml to about 0.1 mg/ml, about 0.01 mg/ml to about 0.5 mg/ml, about 0.01 mg/ml to about 1 mg/ml, about 0.01 mg/ml to about 2 mg/ml, about 0.01 mg/ml to about 3 mg/ml, about 0.01 mg/ml to about 4 mg/ml, about 0.01 mg/ml to about 5 mg/ml, about 0.05 mg/ml to about 0.1 mg/ml, about 0.05 mg/ml to about 0.5 mg/ml, about 0.05 mg/ml to about 1 mg/ml, about 0.05 mg/ml to about 2 mg/ml, about 0.05 mg/ml to about 3 mg/ml, about 0.05 mg/ml to about 4 mg/ml, about 0.05 mg/ml to about 5 mg/ml, about 0.1 mg/ml to about 0.5 mg/ml, about 0.1 mg/ml to about 1 mg/ml, about 0.1 mg/ml to about 2 mg/ml, about 0.1 mg/ml to about 3 mg/ml, about 0.1 mg/ml to about 4 mg/ml, about 0.1 mg/ml to about 5 mg/ml, about 0.5 mg/ml to about 1 mg/ml, about 0.5 mg/ml to about 2 mg/ml, about 0.5 mg/ml to about 3 mg/ml, about 0.5 mg/ml to about 4 mg/ml, about 0.5 mg/ml to about 5 mg/ml, about 1 mg/ml to about 2 mg/ml, about 1 mg/ml to about 3 mg/ml, about 1 mg/ml to about 4 mg/ml, about 1 mg/ml to about 5 mg/ml, about 2 mg/ml to about 3 mg/ml, about 2 mg/ml to about 4 mg/ml, about 2 mg/ml to about 5 mg/ml, about 3 mg/ml to about 4 mg/ml, about 3 mg/ml to about 5 mg/ml, or about 4 mg/ml to about 5 mg/ml.

In some embodiments, the moiety of the agent may bind to the TCR variable region with a dissociation constant (K_{D}). In some embodiments, the moiety can bind to the TCR variable region with a K_{D} of at most about 5000 nM, at most about 1000 nM, at most about 750 nM, at most about 500 nM, at most about 400 nM, at most about 300 nM, at most about 200 nM, at most about 100 nM, at most about 50 nM, at most about 40 nM, at most about 30 nM, at most about 20 nM, at most about 10 nM, at most about 5 nM, at most about 4 nM, at most about 3 nM, at most about 2 nM, at most about 1 nM, at most about 0.1 nM, at most about 0.01 nM, or less than about 0.01 nM. In some embodiments, the moiety can bind to the TCR variable region with a K_{D} of at least about 0.01 nM, at least about 0.1 nM, at least about 1 nM, at least about 2 nM, at least about 3 nM, at least about 4 nM, at least about 5 nM, at least about 10 nM, at least about 20 nM, at least about 30 nM, at least about 40 nM, at least about 50 nM, at least about 100 nM, at least about 200 nM, at least about 300 nM, at least about 400 nM, at least about 500 nM, at least about 750 nM, at least about 1000 nM, at least about 5000 nM, or greater than about 5000 nM. In some embodiments, the moiety can bind to the TCR variable region with a K_{D} between about 0.01 nM to about 500 nM. In some embodiments, the moiety can bind to the TCR variable region with a K_{D} between about 0.01 nM to about 0.1 nM, about 0.01 nM to about 1 nM, about 0.01 nM to about 2 nM, about 0.01 nM to about 3 nM, about 0.01 nM to about 4 nM, about 0.01 nM to about 5 nM, about 0.01 nM to about 10 nM, about 0.01 nM to about 25 nM, about 0.01 nM to about 50 nM, about 0.01 nM to about 100 nM, about 0.01 nM to about 500 nM, about 0.1 nM to about 1 nM, about 0.1 nM to about 2 nM, about 0.1 nM to about 3 nM, about 0.1 nM to about 4 nM, about 0.1 nM to about 5 nM, about 0.1 nM to about 10 nM, about 0.1 nM to about 25 nM, about 0.1 nM to about 50 nM, about 0.1 nM to about 100 nM, about 0.1 nM to about 500 nM, about 1 nM to about 2 nM, about 1 nM to about 3 nM, about 1 nM to about 4 nM, about 1 nM to about 5 nM, about 1 nM to about 10 nM, about 1 nM to about 25 nM, about 1 nM to about 50 nM, about 1 nM to about 100 nM, about 1 nM to about 500 nM, about 2 nM to about 3 nM, about 2 nM to about 4 nM, about 2 nM to about 5 nM, about 2 nM to about 10 nM, about 2 nM to about 25 nM, about 2 nM to about 50 nM, about 2 nM to about 100 nM, about 2 nM to about 500 nM, about 3 nM to about 4 nM, about 3 nM to about 5 nM, about 3 nM to about 10 nM, about 3 nM to about 25 nM, about 3 nM to about 50 nM, about 3 nM to about 100 nM, about 3 nM to about 500 nM, about 4 nM to about 5 nM, about 4 nM to about 10 nM, about 4 nM to about 25 nM, about 4 nM to about 50 nM, about 4 nM to about 100 nM, about 4 nM to about 500 nM, about 5 nM to about 10 nM, about 5 nM to about 25 nM, about 5 nM to about 50 nM, about 5 nM to about 100 nM, about 5 nM to about 500 nM, about 10 nM to about 25 nM, about 10 nM to about 50 nM, about 10 nM to about 100 nM, about 10 nM to about 500 nM, about 25 nM to about 50 nM, about 25 nM to about 100 nM, about 25 nM to about 500 nM, about 50 nM to about 100 nM, about 50 nM to about 500 nM, or about 100 nM to about 500 nM.

Also described herein are methods of treating a subject having an autoimmune disease, comprising: acquiring a status of one or more TCRαV and/or TCRβV molecules in a sample from the subject, and determining the one or more TCRαV and/or TCRβV molecules is expressed in the autoreactive T cell population, in a sample from the subject, wherein responsive to said determination, administering to the subject an effective amount of an anti-TCRαV antibody molecule and/or anti-TCRβV antibody molecule, e.g., an agonistic anti-TCRαV antibody and/or an agonistic anti-TCβaV antibody molecule, e.g., as described herein.

In some embodiments, acquiring a value for the status, e.g., presence, level and/or activity, of one or more TCRαV and/or TCRβV molecules comprises acquiring a measure of the T cell receptor (TCR) repertoire of a sample. In some embodiments, the value comprises a measure of the clonotype of a population of T cells in the sample.

In some embodiments, a value for the status of one or more TCRαV and/or TCRβV molecules is obtained, e.g., measured, using an assay described in Wang C Y, et al., Int J Oncol. (2016) 48(6):2247-56, the entire contents of which are hereby incorporated by reference.

In some embodiments, a value for the status of one or more TCRαV and/or TCRβV molecules is obtained, e.g., measured, using flow cytometry.

In some embodiments, the subject is administered the multifunctional polypeptide molecule as described herein comprising an anti-TCRαV and/or anti-TCRβV binding moiety.

In some embodiments, provided herein is a method of treating a subject that suffers from amyotrophic lateral sclerosis (ALS) by administering to the subject a TCR targeting molecule disclosed herein that specifically binds to a TCRβV7 subfamily molecule, a nucleic acid molecule encoding the TCR targeting molecule, or a cell that expresses the TCR targeting molecule (e.g., a CAR-T cell disclosed herein). In some embodiments, the TCRβV7 subfamily molecule comprises a TCRβV7-1 molecule. In some embodiments, provided herein is a method of treating a subject that suffers from amyotrophic lateral sclerosis (ALS) by administering to the subject a TCR targeting molecule disclosed herein that specifically binds to a TCRβV23 subfamily molecule, a nucleic acid molecule encoding the TCR targeting molecule, or a cell that expresses the TCR targeting molecule (e.g., a CAR-T cell disclosed herein).

In some embodiments, provided herein is a method of treating a subject that suffers from Celiac disease by administering to the subject a TCR targeting molecule disclosed herein that specifically binds to a TCRβV4 subfamily molecule, a nucleic acid molecule encoding the TCR targeting molecule, or a cell that expresses the TCR targeting molecule (e.g., a CAR-T cell disclosed herein). In some embodiments, provided herein is a method of treating a subject that suffers from Celiac disease by administering to the subject a TCR targeting molecule disclosed herein that specifically binds to a TCRβV7 subfamily molecule, a nucleic acid molecule encoding the TCR targeting molecule, or a cell that expresses the TCR targeting molecule (e.g., a CAR-T cell disclosed herein). In some embodiments, provided herein is a method of treating a subject that suffers from Celiac disease by administering to the subject a TCR targeting molecule disclosed herein that specifically binds to a TCRαV4 subfamily molecule, a nucleic acid molecule encoding the TCR targeting molecule, or a cell that expresses the TCR targeting molecule (e.g., a CAR-T cell disclosed herein). In some embodiments, provided herein is a method of treating a subject that suffers from Celiac disease by administering to the subject a TCR targeting molecule disclosed herein that specifically binds to a TCRαV26 subfamily molecule, a nucleic acid molecule encoding the TCR targeting molecule, or a cell that expresses the TCR targeting molecule (e.g., a CAR-T cell disclosed herein).

In some embodiments, provided herein is a method of treating a subject that suffers from ankylosing spondylitis by administering to the subject a TCR targeting molecule disclosed herein that specifically binds to a TCRβV9 subfamily molecule, a nucleic acid molecule encoding the TCR targeting molecule, or a cell that expresses the TCR targeting molecule (e.g., a CAR-T cell disclosed herein). In some embodiments, provided herein is a method of treating a subject that suffers from ankylosing spondylitis by administering to the subject a TCR targeting molecule disclosed herein that specifically binds to a TCRαV21 subfamily molecule, a nucleic acid molecule encoding the TCR targeting molecule, or a cell that expresses the TCR targeting molecule (e.g., a CAR-T cell disclosed herein). In some embodiments, provided herein is a method of treating a subject that suffers from Covid-induced multisystem inflammatory syndrome in children (MIS-C) by administering to the subject a TCR targeting molecule disclosed herein that specifically binds to a TCRβV11 subfamily molecule, a nucleic acid molecule encoding the TCR targeting molecule, or a cell that expresses the TCR targeting molecule (e.g., a CAR-T cell disclosed herein). In some embodiments, the TCRβV11 subfamily molecule comprises a TCRβV11-2 molecule. In some embodiments, provided herein is a method of treating a subject that suffers from primary Sjogren's syndrome by administering to the subject a TCR targeting molecule disclosed herein that specifically binds to a TCRβV6 subfamily molecule, a nucleic acid molecule encoding the TCR targeting molecule, or a cell that expresses the TCR targeting molecule (e.g., a CAR-T cell disclosed herein). In some embodiments, the TCRβV6 subfamily molecule comprises a TCRβV6-1 molecule, a TCRβV6-2/3 molecule, or a TCRβV6-5 molecule.

In some embodiments, provided herein is a method of treating a subject that suffers from multiple sclerosis by administering to the subject a TCR targeting molecule disclosed herein that specifically binds to a TCRβV20 subfamily molecule, a nucleic acid molecule encoding the TCR targeting molecule, or a cell that expresses the TCR targeting molecule (e.g., a CAR-T cell disclosed herein). In some embodiments, the TCRβV20 subfamily molecule is a TCRβV20-1 molecule. In some embodiments, provided herein is a method of treating a subject that suffers from multiple sclerosis by administering to the subject a TCR targeting molecule disclosed herein that specifically binds to a TCRβV29 subfamily molecule, a nucleic acid molecule encoding the TCR targeting molecule, or a cell that expresses the TCR targeting molecule (e.g., a CAR-T cell disclosed herein). In some embodiments, the TCRβV20 subfamily molecule is a TCRβV29-1 molecule. In some embodiments, provided herein is a method of treating a subject that suffers from multiple sclerosis by administering to the subject a TCR targeting molecule disclosed herein that specifically binds to a TCRβV7 subfamily molecule, a nucleic acid molecule encoding the TCR targeting molecule, or a cell that expresses the TCR targeting molecule (e.g., a CAR-T cell disclosed herein). In some embodiments, the TCRβV7 subfamily molecule is a TCRβV7-8/9 molecule. In some embodiments, provided herein is a method of treating a subject that suffers from multiple sclerosis by administering to the subject a TCR targeting molecule disclosed herein that specifically binds to a TCRβV10 subfamily molecule, a nucleic acid molecule encoding the TCR targeting molecule, or a cell that expresses the TCR targeting molecule (e.g., a CAR-T cell disclosed herein). In some embodiments, the TCRβV10 subfamily molecule is a TCRβV10-3 molecule. In some embodiments, provided herein is a method of treating a subject that suffers from multiple sclerosis by administering to the subject a TCR targeting molecule disclosed herein that specifically binds to a TCRβV6 subfamily molecule, a nucleic acid molecule encoding the TCR targeting molecule, or a cell that expresses the TCR targeting molecule (e.g., a CAR-T cell disclosed herein).

In some embodiments, provided herein is a method of treating a subject that suffers from type I diabetes by administering to the subject a TCR targeting molecule disclosed herein that specifically binds to a TCRβV12 subfamily molecule, a nucleic acid molecule encoding the TCR targeting molecule, or a cell that expresses the TCR targeting molecule (e.g., a CAR-T cell disclosed herein). In some embodiments, the TCRβV12 subfamily molecule comprises a TCRβV12-3/4 molecule. In some embodiments, provided herein is a method of treating a subject that suffers from type I diabetes by administering to the subject a TCR targeting molecule disclosed herein that specifically binds to a TCRαV12 subfamily molecule, a nucleic acid molecule encoding the TCR targeting molecule, or a cell that expresses the TCR targeting molecule (e.g., a CAR-T cell disclosed herein). In some embodiments, the TCRαV12 subfamily molecule comprises a TCRαV12-3 molecule.

In some embodiments, a subject has amyotrophic lateral sclerosis. In some embodiments, the subject having amyotrophic lateral sclerosis has a population of autoreactive T cells that express a TCRβV7 subfamily comprising TCRβV7-1. In some embodiments, the subject is administered the agent, antibody molecule, multispecific molecule, and/or multifunctional molecule as described herein comprising an anti-TCRβV molecule that binds to one or more members of the TCRβV7 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV7 subfamily. In some embodiments, administration of the agent, antibody molecule, multispecific molecule, and/or multifunctional molecule as described herein results in reducing or depleting the population of autoreactive T cells expressing one or more members of the TCRβV7 subfamily. In some embodiments, the subject having amyotrophic lateral sclerosis has a population of autoreactive T cells that express a TCRβV23 subfamily. In some embodiments, the subject is administered the agent, antibody molecule, multispecific molecule, and/or multifunctional molecule as described herein comprising an anti-TCRβV molecule that binds to one or more members of the TCRβV23 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV23 subfamily. In some embodiments, administration of the agent, antibody molecule, multispecific molecule, and/or multifunctional molecule as described herein results in reducing or depleting the population of autoreactive T cells expressing one or more members of the TCRβV23 subfamily. In some embodiments, the subject having amyotrophic lateral sclerosis has a population of autoreactive T cells that express a TCRβV13 subfamily. In some embodiments, the subject is administered the agent, antibody molecule, multispecific molecule, and/or multifunctional molecule as described herein comprising an anti-TCRβV molecule that binds to one or more members of the TCRβV13 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV13 subfamily. In some embodiments, administration of the agent, antibody molecule, multispecific molecule, and/or multifunctional molecule as described herein results in reducing or depleting the population of autoreactive T cells expressing one or more members of the TCRβV13 subfamily

In some embodiments, a subject has Celiac disease. In some embodiments, the subject having Celiac disease has a population of autoreactive T cells that express a TCRβV4 subfamily. In some embodiments, the subject is administered the agent, antibody molecule, multispecific molecule, and/or multifunctional molecule as described herein comprising an anti-TCRβV molecule that binds to one or more members of the TCRβV4 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV4 subfamily. In some embodiments, administration of the agent, antibody molecule, multispecific molecule, and/or multifunctional molecule as described herein results in reducing or depleting the population of autoreactive T cells expressing one or more members of the TCRβV4 subfamily. In some embodiments, the subject having Celiac disease has a population of autoreactive T cells that express a TCRβV7 subfamily. In some embodiments, the subject is administered the agent, antibody molecule, multispecific molecule, and/or multifunctional molecule as described herein comprising an anti-TCRαV molecule that binds to one or more members of the TCRβV7 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV7 subfamily. In some embodiments, administration of the agent, antibody molecule, multispecific molecule, and/or multifunctional molecule as described herein results in reducing or depleting the population of autoreactive T cells expressing one or more members of the TCRβV7 subfamily. In some embodiments, the subject having Celiac disease has a population of autoreactive T cells that express a TCRαV4 subfamily. In some embodiments, the subject is administered the agent, antibody molecule, multispecific molecule, and/or multifunctional molecule as described herein comprising an anti-TCRαV molecule that binds to one or more members of the TCRαV4 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRαV4 subfamily. In some embodiments, administration of the agent, antibody molecule, multispecific molecule, and/or multifunctional molecule as described herein results in reducing or depleting the population of autoreactive T cells expressing one or more members of the TCRαV4 subfamily. In some embodiments, the subject having Celiac disease has a population of autoreactive T cells that express a TCRαV26 subfamily. In some embodiments, the subject is administered the agent, antibody molecule, multispecific molecule, and/or multifunctional molecule as described herein comprising an anti-TCRαV molecule that binds to one or more members of the TCRαV26 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRαV26 subfamily. In some embodiments, administration of the agent, antibody molecule, multispecific molecule, and/or multifunctional molecule as described herein results in reducing or depleting the population of autoreactive T cells expressing one or more members of the TCRαV26 subfamily.

In some embodiments, a subject has ankylosing spondylitis. In some embodiments, the subject having ankylosing spondylitis has a population of autoreactive T cells that express a TCRβV9 subfamily. In some embodiments, the subject is administered the agent, antibody molecule, multispecific molecule, and/or multifunctional molecule as described herein comprising an anti-TCRβV molecule that binds to one or more members of the TCRβV9 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV9 subfamily. In some embodiments, administration of the agent, antibody molecule, multispecific molecule, and/or multifunctional molecule as described herein results in reducing or depleting the population of autoreactive T cells expressing one or more members of the TCRβV9 subfamily. In some embodiments, the subject having ankylosing spondylitis has a population of autoreactive T cells that express a TCRαV21 subfamily. In some embodiments, the subject is administered the agent, antibody molecule, multispecific molecule, and/or multifunctional molecule as described herein comprising an anti-TCRαV molecule that binds to one or more members of the TCRαV21 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRαV21 subfamily. In some embodiments, administration of the agent, antibody molecule, multispecific molecule, and/or multifunctional molecule as described herein results in reducing or depleting the population of autoreactive T cells expressing one or more members of the TCRαV21 subfamily.

In some embodiments, a subject has Covid-induced multisystem inflammatory syndrome in children (MIS-C). In some embodiments, the subject having MIS-C has a population of autoreactive T cells that express a TCRβV11 subfamily comprising TCRβV11-2. In some embodiments, the subject is administered the agent, antibody molecule, multispecific molecule, and/or multifunctional molecule as described herein comprising an anti-TCRβV molecule that binds to one or more members of the TCRβV11 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV11 subfamily. In some embodiments, administration of the agent, antibody molecule, multispecific molecule, and/or multifunctional molecule as described herein results in reducing or depleting the population of autoreactive T cells expressing one or more members of the TCRβV11 subfamily.

In some embodiments, a subject has primary Sjogren's syndrome. In some embodiments, the subject having primary Sjogren's syndrome has a population of autoreactive T cells that express a TCRβV6 subfamily comprising TCRβV6-1, TCRβV6-2/3, or TCRβV6-5. In some embodiments, the subject is administered the agent, antibody molecule, multispecific molecule, and/or multifunctional molecule as described herein comprising an anti-TCRβV molecule that binds to one or more members of the TCRβV6 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV6 subfamily. In some embodiments, administration of the agent, antibody molecule, multispecific molecule, and/or multifunctional molecule as described herein results in reducing or depleting the population of autoreactive T cells expressing one or more members of the TCRβV6 subfamily.

In some embodiments, a subject has multiple sclerosis. In some embodiments, the subject having multiple sclerosis has a population of autoreactive T cells that express a TCRβV20 subfamily comprising TCRβV20-1. In some embodiments, the subject is administered the agent, antibody molecule, multispecific molecule, and/or multifunctional molecule as described herein comprising an anti-TCRβV molecule that binds to one or more members of the TCRβV20 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV20 subfamily. In some embodiments, administration of the agent, antibody molecule, multispecific molecule, and/or multifunctional molecule as described herein results in reducing or depleting the population of autoreactive T cells expressing one or more members of the TCRβV20 subfamily. In some embodiments, the subject having multiple sclerosis has a population of autoreactive T cells that express a TCRβV29 subfamily comprising TCRβV29-1. In some embodiments, the subject is administered the agent, antibody molecule, multispecific molecule, and/or multifunctional molecule as described herein comprising an anti-TCRβV molecule that binds to one or more members of the TCRβV29 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV29 subfamily. In some embodiments, administration of the agent, antibody molecule, multispecific molecule, and/or multifunctional molecule as described herein results in reducing or depleting the population of autoreactive T cells expressing one or more members of the TCRβV29 subfamily. In some embodiments, the subject having multiple sclerosis has a population of autoreactive T cells that express a TCRβV7 subfamily comprising TCRβV7-8/9. In some embodiments, the subject is administered the agent, antibody molecule, multispecific molecule, and/or multifunctional molecule as described herein comprising an anti-TCRβV molecule that binds to one or more members of the TCRβV7 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV7 subfamily. In some embodiments, administration of the agent, antibody molecule, multispecific molecule, and/or multifunctional molecule as described herein results in reducing or depleting the population of autoreactive T cells expressing one or more members of the TCRβV7 subfamily. In some embodiments, the subject having multiple sclerosis has a population of autoreactive T cells that express a TCRβV10 subfamily comprising TCRβV10-3. In some embodiments, the subject is administered the agent, antibody molecule, multispecific molecule, and/or multifunctional molecule as described herein comprising an anti-TCRβV molecule that binds to one or more members of the TCRβV10 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV10 subfamily. In some embodiments, administration of the agent, antibody molecule, multispecific molecule, and/or multifunctional molecule as described herein results in reducing or depleting the population of autoreactive T cells expressing one or more members of the TCRβV10 subfamily. In some embodiments, the subject having multiple sclerosis has a population of autoreactive T cells that express a TCRβV6 subfamily. In some embodiments, the subject is administered the agent, antibody molecule, multispecific molecule, and/or multifunctional molecule as described herein comprising an anti-TCRβV molecule that binds to one or more members of the TCRβV6 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV6 subfamily. In some embodiments, administration of the agent, antibody molecule, multispecific molecule, and/or multifunctional molecule as described herein results in reducing or depleting the population of autoreactive T cells expressing one or more members of the TCRβV6 subfamily.

In some embodiments, a subject has type I diabetes. In some embodiments, the subject having type I diabetes has a population of autoreactive T cells that express a TCRβV12 subfamily comprising TCRβV12-3/4. In some embodiments, the subject is administered the agent, antibody molecule, multispecific molecule, and/or multifunctional molecule as described herein comprising an anti-TCRβV molecule that binds to one or more members of the TCRβV12 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV12 subfamily. In some embodiments, administration of the agent, antibody molecule, multispecific molecule, and/or multifunctional molecule as described herein results in reducing or depleting the population of autoreactive T cells expressing one or more members of the TCRβV12 subfamily. In some embodiments, the subject having type I diabetes has a population of autoreactive T cells that express a TCRαV12 subfamily comprising TCRαV12-3. In some embodiments, the subject is administered the agent, antibody molecule, multispecific molecule, and/or multifunctional molecule as described herein comprising an anti-TCRαV molecule that binds to one or more members of the TCRαV12 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRαV12 subfamily. In some embodiments, administration of the agent, antibody molecule, multispecific molecule, and/or multifunctional molecule as described herein results in reducing or depleting the population of autoreactive T cells expressing one or more members of the TCRαV12 subfamily.

In some embodiments, a subject has acute anterior uveitis (UAA). In some embodiments, the subject having acute anterior uveitis has a population of autoreactive T cells that express a TCRβV9 subfamily. In some embodiments, the subject is administered the agent, antibody molecule, multispecific molecule, and/or multifunctional molecule as described herein comprising an anti-TCRβV molecule that binds to one or more members of the TCRβV9 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV9 subfamily. In some embodiments, administration of the agent, antibody molecule, multispecific molecule, and/or multifunctional molecule as described herein results in reducing or depleting the population of autoreactive T cells expressing one or more members of the TCRβV9 subfamily. In some embodiments, the subject having acute anterior uveitis has a population of autoreactive T cells that express a TCRαV21 subfamily. In some embodiments, the subject is administered the agent, antibody molecule, multispecific molecule, and/or multifunctional molecule as described herein comprising an anti-TCRαV molecule that binds to one or more members of the TCRαV21 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRαV21 subfamily. In some embodiments, administration of the agent, antibody molecule, multispecific molecule, and/or multifunctional molecule as described herein results in reducing or depleting the population of autoreactive T cells expressing one or more members of the TCRαV21 subfamily.

In some embodiments, provided herein is a method of treating a subject that suffers from acute anterior uveitis (UAA) by administering to the subject a TCR targeting molecule disclosed herein that specifically binds to a TCRβV9 subfamily molecule, a nucleic acid molecule encoding the TCR targeting molecule, or a cell that expresses the TCR targeting molecule (e.g., a CAR-T cell disclosed herein). In some embodiments, provided herein is a method of treating a subject that suffers from acute anterior uveitis (UAA) by administering to the subject a TCR targeting molecule disclosed herein that specifically binds to a TCRαV21 subfamily molecule, a nucleic acid molecule encoding the TCR targeting molecule, or a cell that expresses the TCR targeting molecule (e.g., a CAR-T cell disclosed herein).

In some embodiments, a subject has psoriatic arthritis (PsA). In some embodiments, the subject having psoriatic arthritis (PsA) has a population of autoreactive T cells that express a TCRβV9 subfamily. In some embodiments, the subject is administered the agent, antibody molecule, multispecific molecule, and/or multifunctional molecule as described herein comprising an anti-TCRβV molecule that binds to one or more members of the TCRβV9 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV9 subfamily. In some embodiments, administration of the agent, antibody molecule, multispecific molecule, and/or multifunctional molecule as described herein results in reducing or depleting the population of autoreactive T cells expressing one or more members of the TCRβV9 subfamily.

In some embodiments, provided herein is a method of treating a subject that suffers from psoriatic arthritis (PsA) by administering to the subject a TCR targeting molecule disclosed herein that specifically binds to a TCRβV9 subfamily molecule, a nucleic acid molecule encoding the TCR targeting molecule, or a cell that expresses the TCR targeting molecule (e.g., a CAR-T cell disclosed herein).

In some embodiments, a subject has type 1 narcolepsy. In some embodiments, the subject having type 1 narcolepsy has a population of autoreactive T cells that express a TCRβV4 subfamily. In some embodiments, the subject having type 1 narcolepsy has a population of autoreactive T cells that express a TCRβV4 subfamily comprising TCRβV4-2. In some embodiments, the subject is administered the agent, antibody molecule, multispecific molecule, and/or multifunctional molecule as described herein comprising an anti-TCRβV molecule that binds to one or more members of the TCRβV4 subfamily. In some embodiments, administration of the agent, antibody molecule, multispecific molecule, and/or multifunctional molecule as described herein results in inhibiting or preventing the activation and/or expansion of the population of autoreactive T cells expressing one or more members of the TCRβV4 subfamily. In some embodiments, administration of the agent, antibody molecule, multispecific molecule, and/or multifunctional molecule as described herein results in reducing or depleting the population of autoreactive T cells expressing one or more members of the TCRβV4 subfamily (e.g., the TCRβV4 subfamily comprising TCRβV4-2).

In some embodiments, provided herein is a method of treating a subject that suffers from type 1 narcolepsy by administering to the subject a TCR targeting molecule disclosed herein that specifically binds to a TCRβV4 subfamily molecule (e.g., a TCRβV4-2 subfamily molecule), a nucleic acid molecule encoding the TCR targeting molecule, or a cell that expresses the TCR targeting molecule (e.g., a CAR-T cell disclosed herein).

In some aspects, the present disclosure provides methods for depleting or reducing T cells in a subject in need thereof. In some embodiments, the T cells are autoreactive T cells. In some embodiments, the autoreactive T cells target autologous T cells of the subject. In some embodiments, the autoreactive T cells express TCRαV, TCRβV, or any combination thereof. In some embodiments, the subject suffers from an autoimmune disease. In some embodiments, the methods comprise administering to the subject an agent comprising a moiety. In some embodiments, the moiety binds to a TCR variable region.

In some embodiments, administering the agent depletes a T cell population in a subject by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or greater than about 99% compared to a baseline measure of the T cell population (e.g., a measure of T cell population prior to administering of the agent). In some embodiments, administering the agent depletes a T cell population in a subject by at most about 99%, at most about 98%, at most about 97%, at most about 96%, at most about 95%, at most about 94%, at most about 93%, at most about 92%, at most about 91%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, or less than about 20% compared to a baseline measure of the T cell population (e.g., a measure of T cell population prior to administering of the agent).

In some embodiments, administering the agent depletes a T cell population in a subject from about 20% to about 99% compared to a baseline measure of the T cell population (e.g., a measure of T cell population prior to administering of the agent). In some embodiments, administering the agent depletes a T cell population in a subject from about 20% to about 30%, about 20% to about 40%, about 20% to about 50%, about 20% to about 60%, about 20% to about 70%, about 20% to about 75%, about 20% to about 80%, about 20% to about 85%, about 20% to about 90%, about 20% to about 95%, about 20% to about 99%, about 30% to about 40%, about 30% to about 50%, about 30% to about 60%, about 30% to about 70%, about 30% to about 75%, about 30% to about 80%, about 30% to about 85%, about 30% to about 90%, about 30% to about 95%, about 30% to about 99%, about 40% to about 50%, about 40% to about 60%, about 40% to about 70%, about 40% to about 75%, about 40% to about 80%, about 40% to about 85%, about 40% to about 90%, about 40% to about 95%, about 40% to about 99%, about 50% to about 60%, about 50% to about 70%, about 50% to about 75%, about 50% to about 80%, about 50% to about 85%, about 50% to about 90%, about 50% to about 95%, about 50% to about 99%, about 60% to about 70%, about 60% to about 75%, about 60% to about 80%, about 60% to about 85%, about 60% to about 90%, about 60% to about 95%, about 60% to about 99%, about 70% to about 75%, about 70% to about 80%, about 70% to about 85%, about 70% to about 90%, about 70% to about 95%, about 70% to about 99%, about 75% to about 80%, about 75% to about 85%, about 75% to about 90%, about 75% to about 95%, about 75% to about 99%, about 80% to about 85%, about 80% to about 90%, about 80% to about 95%, about 80% to about 99%, about 85% to about 90%, about 85% to about 95%, about 85% to about 99%, about 90% to about 95%, about 90% to about 99%, or about 95% to about 99% compared to a baseline measure of the T cell population (e.g., a measure of T cell population prior to administering of the agent).

In some embodiments, administering the agent to a subject in need thereof provides sustained reducing and/or depletion of a targeted T cell or T cell population. In some embodiments, administering the agent to a subject in need thereof can deplete T cells for at least about 30 minutes, at least about 1 hour, at least about 2 hours, at least about 3 hours, at least about 4 hours, at least about 5 hours, at least about 6 hours, at least about 12 hours, at least about 1 day, at least about 2 days, at least about 3 days, at least about 4 days, at least about 5 days, at least about 6 days, at least about 7 days, at least about 2 weeks, at least about 3 weeks, at least about 4 weeks, at least about 2 months, at least about 3 months, at least about 4 months, at least about 5 months, at least about 6 months, or greater than about 6 months.

The methods herein may also provide identifying a subject based on a TCR clonotype. Identifying a subject may comprise genotyping a subject who suffers from a disease or condition. The disease may be an autoimmune disease. The autoimmune disease may be selected from the group consisting of amyotrophic lateral sclerosis (ALS), coeliac disease (CD), ankylosing spondylitis (AS), Covid-induced multisystem inflammatory syndrome in children (MIS-C), primary Sjogren's syndrome (PSS), Churg-Strauss syndrome, sarcoidosis, systemic lupus erythematosus (SLE), type 1 diabetes, autoimmune hepatitis (e.g., type 1 or type 2), primary sclerosing cholangitis, primary biliary cirrhosis, multiple sclerosis, Guillain-Barre syndrome and the AMAN (axonal & neuronal neuropathy), chronic inflammatory demyelinating polyneuropathy (CIDP), transverse myelitis, Tolosa-Hunt syndrome (THS), Devic's disease (neuromyelitis optica), paraneoplastic cerebellardegeneration (PCD), Lambert-Eaton syndrome, psoriasis, scleroderma, CREST (calcinosis,Raynaud phenomenon, esophageal dysmotility, sclerodactyly, and telangiectasia) syndrome, dermatitis herpetiformis, dermatomyositis, bullous pemphigoid, cicatricial pemphigoid/benign mucosal pemphigoid, pemphigoid gestationis, rheumatoid arthritis (RA), psoriatic arthritis, relapsing polychondritis, chronic recurrent multifocal osteomyelitis (CRMO), vasculitis, Kawasaki disease, granulomatosis with polyangiitis (GPA), Behcet's disease (vasculitis),Takayasu's arteritis, polyarteritis nodosa, microscopic polyangiitis (MPA), joint implant failure, leukocytoclasticvasculitis, Cogan's syndrome, uveitis, peripheral uveitis (Pars planitis), scleritis, autoimmune inner ear disease (AIED), Crohn's, ulcerative colitis (UC), Dressler's syndrome, Rheumatic fever, Evans syndrome, paroxysmal nocturnal hemoglobinuria (PNH), hemolytic anemia, COVID-induced multisystem inflammatory syndrome thrombocytopenic purpura (TTP), polymyositis, juvenile myositis (JM), including Juvenile Dermatomyositis (JDM) and Juvenile Polymyositis (JPM), ocular cicatricial pemphigoid, or Hashimoto's thyroiditis.

A sample may be obtained from a subject and sequenced to determine a TCR clonotype associated with the autoimmune disease. The TCR clonotype can comprise a T cell receptor alpha variable region (TCRαV) or a T cell receptor variable beta region (TCRβV). In some embodiments, the genotype may be determined. In some embodiments, commercial methodologies available for genotyping, for example, SNP genotyping, can be used, but are not limited to, TaqMan genotyping assays (Applied Biosystems), SNPlex platforms (Applied Biosystems), gel electrophoresis, capillary electrophoresis, size exclusion chromatography, mass spectrometry, for example, MassARRAY system (Sequenom), minisequencing methods, real-time Polymerase Chain Reaction (PCR), Bio-Plex system (BioRad), CEQ and SNPstream systems (Beckman), array hybridization technology, for example, Affymetrix GeneChip (Perlegen), BeadArray Technologies, for example, Illumina GoldenGate and Infinium assays, array tag technology, Multiplex Ligation-dependent Probe Amplification (MLPA), and endonuclease-based fluorescence hybridization technology (Invader assay, either using unamplified or amplified genomic DNA, or unamplified total RNA, or unamplified or amplified cDNA; Third Wave/Hologic).

### EXAMPLES

The following examples are included for illustrative purposes only and are not intended to limit the scope of the invention.

### Example 1: Immunization of Armenian Hamster to Generate Anti-NKp30 Antibodies

Briefly, armenian hamsters were immunized with the extracellular domain of human NKp30 protein in complete Freund's adjuvant and boosted twice on day 14 and day 28 with NKp30 in incomplete Freund's adjuvant (IFA). On day 56 one more boost in IFA was given and the animals harvested three days later. Spleens were collected and fused with P3X63Ag8.653 murine myeloma cell line. 0.9×10\vec{ }5 cells/well in 125 μl were seated in 96 well plate and fed with 125 μl of I-20+2ME+HAT (IMDM (4 g/L glucose) supplemented with 20% fetal bovine serum, 4 mM L-glutamine, 1 mM sodium pyruvate, 50 U penicillin, 50 g streptomycin and 50 M 2-ME in the absence or presence of HAT or HT for selection, and Hybridoma Cloning Factor (1% final) on days 7, 11 and thereafter as needed. At approximately 2 weeks after fusion (cells are about 50% confluent), supernatant was collected and assayed for binding.

### Example 2: Hybridoma Screen for NKp30 mAbs

Expi293 cells were transfected with BG160 (hNKp30 cell antigen) 18 hours prior to screening. The day of screening, transfected cells were diluted to 0.05×10^{\hat{ }6}/mL and anti-Armenian hamster Fc Alexa Fluor 488 added to a final concentration of 0.4 ug/mL. 50 uL (2,500 cells) of this mixture was added to each well of a 384 well plate. The same density of untransfected 293 cells with secondary were used as a negative control. 5 μL of hybridoma supernatant was added to the cell mixture and the plate incubated for 1 hour at 37° C. The plates were then imaged on Mirrorball. Positive clones were identified and subcloned by serial dilution to obtain clonal selected hybridoma. After reconfirmation using the same protocols the hybridoma cells were harvested and the corresponding heavy and light chain sequences recovered. The DNA was subcloned into pcDNA3.4 for subsequent expression of the corresponding antibodies and further validation.

### Example 3: Binding of NKp30 Antibodies to NK92 Cells

NK-92 cells were washed with PBS containing 0.5% BSA and 0.1% sodium azide (staining buffer) and added to 96-well V-bottom plates with 200,000 cells/well. Hamster NKp30 antibodies were added to the cells in 2.0 fold serial dilutions and incubated for 1 hour at room temperature. The plates were washed twice with staining buffer. The secondary antibody against hamster Fc conjugated to AF647 (Jackson, 127-605-160) was added at 1:100 dilution (1.4 mg/ml stock) and incubated with the cells for 30 minutes at 4° C. followed by washing with staining buffer. Cells were subsequently fixed for 10 minutes with 4% paraformaldehyde at room temperature. The plates were read on CytoFLEX LS (Beckman Coulter).

### Example 4: Bioassay to Measure Activity of NKp30 Antibodies Using NK92 Cell Line

NKp30 antibodies were three-fold serially diluted in PBS and incubated at 2-8° C. overnight in flat bottom 96 well plates. Plates were washed twice in PBS and 40,000 NK-92 cells were added in growth medium containing IL-2. Plates were incubated at 37° C., 5% CO2, humidified incubator for 16-24 hours before supernatants were collected. IFNγ levels in supernatants was measured following MSD assay instructions. Supernatant collected from cells incubated with hamster isotype IgG was used as negative control and supernatants from cells incubated with NKp30 monoclonal antibody (R&D, clone 210847) was utilized as a positive control. Data were generated using hamster anti-NKp30 mABs.

### Example 5: Generation and Characterization of Humanized Anti-NKp30 Antibodies

A series of hamster anti-NKp30 antibodies were selected. These antibodies were shown to bind to human NKp30 and cynomolgus NKp30 and induce IFNγ production from NK-90 cells (data not shown). The VH and VL sequences of exemplary hamster anti-NKp30 antibodies 15E1, 9G1, 15H6, 9D9, 3A12, and 12D10 are disclosed in Table 25. The VH and VL sequences of exemplary humanized anti-NKp30 antibodies based on 15E1, 9G1, and 15H6 are also disclosed in Table 25 and Table 40. The Kabat CDRs of these antibodies are disclosed in Table 21A and Table 23A.

Two humanized constructs based on 15E1 were selected. The first construct BJM0407 is a Fab comprising a heavy chain variable region comprising the amino acid sequence of SEQ ID NO: 7302 and a lambda light chain variable region comprising the amino acid sequence of SEQ ID NO: 7305. Its corresponding scFv construct BJM0859 comprises the amino acid sequence of SEQ ID NO: 7310. The second construct BJM0411 is a Fab comprising a heavy chain variable region comprising the amino acid sequence of SEQ ID NO: 7302 and a kappa light chain variable region comprising the amino acid sequence of SEQ ID NO: 13. Its corresponding scFv construct BJM0860 comprises the amino acid sequence of SEQ ID NO: 7311. BJM0407 and BJM0411 showed comparable biophysical characteristics, e.g., binding affinity to NKp30 and thermal stability. The scFv constructs BJM0859 and BJM0860 also showed comparable biophysical properties.

### Example 6: Biacore Analysis of Exemplary Anti-NKp30 Antibody Molecules

In this example, a series of exemplary anti-NKp30 antibody molecules were analyzed for their binding affinity for human and cynomolgus NKp30. Briefly, surface plasmon resonance (SPR) measurements were performed by using the BIAcore T200. Human NKp30 (BKM0179) was immobilized on a CM5 chip via anti-mouse Fe antibody to a response of 50 RU. Cynomolgus NKp30 (BKM0179) was immobilized on a CM5 chip via anti-mouse Fe antibody to a response of 50 RU. Each exemplary antibody construct were injected at concentrations of 3.9, 7.8, 15.6, 31.2, 62.5, and 125 nM, and at a flow rate of 20 μl/min, over the surface on which the human NKp30 or cynomolgus NKp30 was immobilized. The data was fit using a 1:1 binding model.

As shown in Table 34, most of the exemplary antibodies showed preserved affinity to human NKp30 compared to the parental antibody.

TABLE 34
Exemplary binding affinities of exemplary molecule comprising
an antigen binding domain that binds to TRBC1 and Nkp30

| | | Human Nkp30 |
|---|---|---|
| Construct | Description | (BKM0179) (K_{D}) |
| BJM1078 | BJM0407 Parental | 1.48 nM |
| BJM1079 | BJM0411 Parental | 1.26 nM |
| BKM0138 | BJM0411 VL-N95A | 3.2 nM |
| BKM0139 | BJM0411 VL-D92A | 3.2 nM |
| BKM0140 | BJM0407 VL-D92A | 3.3 nM |
| BKM0141 | BJM0407 VL-N95A | 3.0 nM |
| BKM0142 | BJM0411 VH-N60A | 1.28 nM |
| BKM0143 | BJM0407 VH-N60A | 1.45 nM |
| BKM0144 | BJM0411 VH-N60A- | 6.4 nM |
| | VL-D92A-N95A | |
| BKM0145 | BJM0407 VH-N60A- | 4.2 nM |
| | VL-D92A-N95A | |

Table 35 shows the binding affinity of BLM0230 to human NKp30 and cynomolgus NKp30. Molecule BLM0230 comprises a VHCDR1 comprising the sequence of SEQ ID NO: 502, a VHCDR2 of the sequence of SEQ ID NO: 503, a VHCDR3 comprising the sequence of SEQ ID NO: 504, a VLCDR1 comprising the sequence of SEQ ID NO: 26, a VLCDR2 comprising the sequence of SEQ ID NO: 28, and a VLCDR3 comprising the sequence of SEQ ID NO: 86.

Molecule BLM0230 comprises a VH of SEQ ID NO: 505 and a VL of SEQ ID NO: 13.

TABLE 35
Exemplary binding affinities of exemplary molecule comprising
an antigen binding domain that binds to Nkp30

| Construct | Human Nkp30 (K_{D}) | Cyno Nkp30 (K_{D}) |
|---|---|---|
| BLM0230 | 22 nM | 17 nM |

### Example 7: Biacore Analysis of Exemplary Anti-TRBC1 Antibody Molecules

In this example, a series of exemplary anti-TRBC1 antibody molecules were analyzed for their binding affinity for TRBC1. Briefly, surface plasmon resonance (SPR) measurements were performed by using the BIAcore T200. Each of the exemplary anti-TRBC1 antibody constructs was immobilized on a CM5 chip via anti-human Fe antibody to a response of 50 RU. Human TRBC1 (BIM0443) was injected at concentrations of 15.6, 31.2, 62.5, 125, 250, and 500 nM, at a flow rate of 20 μl/min, over the surface on which each antibody construct was immobilized. The data was fit using a 1:1 binding model.

As shown in Table 33, the exemplary antibodies showed preserved affinity to human TRCB1 compared to the parental antibody.

TABLE 33
Exemplary binding affinities of exemplary molecule comprising an
antigen binding domain that binds to TRBC1 and Nkp30

| | | BIM0443 | BIM0445 |
|---|---|---|---|
| Construct | Description | (hTRBC1) (K_{D}) | (hTRBC2) (K_{D}) |
| BIM0460 (bivalent) | Parental | 10 nM | No binding |
| BJM0578 (bivalent) | Parental | 75 nM | No binding |
| BKM0191 (single arm) | G29A | 75 nM | No binding |
| BKM0192 (single arm) | T31A | 138 nM | No binding |
| BKM0193 (single arm) | G29A_T31A | 360 nM | No binding |
| BKM0194 (single arm) | N28G | 180 nM | No binding |
| BKM0195 (single arm) | N30S | 317 nM | No binding |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

TABLE 1
Exemplary Fc KiH mutations
and optional Cysteine mutations

| Position | Knob Mutation | Hole Mutation |
|---|---|---|
| T366 | T366W | T366S |
| L368 | — | L368A |
| Y407 | — | Y407V |

Additional Cysteine Mutations to
form a stabilizing disulfide bridge

| Position | Knob CH3 | Hole CH3 |
|---|---|---|
| S354 | S354C | — |
| Y349 | — | Y349C |

TABLE 2A
Exemplary heavy chain CDRs and FWRs of TRBC1-targeting
antigen binding domains derived from JOVI.1
(according to Kabat numbering convention)

| AB ID | VHFWR1 | VHCDR1 | VHFWR2 | VHCDR2 | VHFWR3 | VHCDR3 | VHFWR4 |
|---|---|---|---|---|---|---|---|
| mJOVI.1- | EVRLQQS | GYVMH | WVKQR | FINPYN | KATLTSD | GAGYNFD | WGQGTTLT |
| H | GPDLIKP | (SEQ ID | PGQGLE | DDIQSN | KSSTTAY | GAYRFFDF | VSS (SEQ |
| | GASVKM | NO: | WIG | ERFRG | MELSSLTS | (SEQ ID | ID NO: 206) |
| | SCKASGY | 8643) | (SEQ ID | (SEQ ID | EDSAVYY | NO: 202) | |
| | TFT (SEQ | | NO: 204) | NO: 201) | CAR (SEQ | | |
| | ID NO: | | | | ID NO: | | |
| | 203) | | | | 8679) | | |
| h1JOVI.1- | QVQLVQ | GYVMH | WVRQA | FINPYN | RVTMTSD | GAGYNFD | WGQGTLV |
| H | SGAEVK | (SEQ ID | PGQGLE | DDIQSN | KSTTTAY | GAYRFFDF | TVSS (SEQ |
| | KPGASVK | NO: | WMG | ERFRG | MELSSLRS | (SEQ ID | ID NO: 210) |
| | VSCKASG | 8643) | (SEQ ID | (SEQ ID | EDTAVYY | NO: 202) | |
| | YTFT | | NO: 208) | NO: 201) | CAR (SEQ | | |
| | (SEQ ID | | | | ID NO: | | |
| | NO: 207) | | | | 209) | | |
| h2JOVI.1- | QVQLVQ | GYVMH | WVRQA | FINPYN | WVTMTSD | GAGYNFD | WGQGTLV |
| H | SGAEVK | (SEQ ID | PGQGLE | DDIQSN | KSITTAY | GAYRFFDF | TVSS (SEQ |
| | KPGASVK | NO: | WMG | ERFRG | MELSRLR | (SEQ ID | ID NO: 210) |
| | VSCKASG | 8643) | (SEQ ID | (SEQ ID | SDDTAVY | NO: 202) | |
| | YTFT | | NO: 208) | NO: 201) | YCAR | | |
| | (SEQ ID | | | | (SEQ ID | | |
| | NO: 207) | | | | NO: 213) | | |
| h3JOVI.1- | QVQLVQ | GYVMH | WVRQA | FINPYN | RVTITSDK | GAGYNFD | WGQGTLV |
| H | SGAEVK | (SEQ ID | PGQGLE | DDIQSN | STTTAYM | GAYRFFDF | TVSS (SEQ |
| | KPGSSVK | NO: | WMG | ERFRG | ELSSLRSE | (SEQ ID | ID NO: 210) |
| | VSCKASG | 8643) | (SEQ ID | (SEQ ID | DTAVYYC | NO: 202) | |
| | YTFT | | NO: 208) | NO: 201) | AR (SEQ | | |
| | (SEQ ID | | | | ID NO: | | |
| | NO: 7345) | | | | 217) | | |
| h4JOVI.1- | QVQLVQ | GYVMH | WVRQA | FINPYN | RVTITSDK | GAGYNFD | WGQGTLV |
| H | SGAEVK | (SEQ ID | PGQRLE | DDIQSN | SATTAYM | GAYRFFDF | TVSS (SEQ |
| | KPGASVK | NO: | WMG | ERFRG | ELSSLRSE | (SEQ ID | ID NO: 210) |
| | VSCKASG | 8643) | (SEQ ID | (SEQ ID | DTAVYYC | NO: 202) | |
| | YTFT | | NO: 212) | NO: 201) | AR (SEQ | | |
| | (SEQ ID | | | | ID NO: | | |
| | NO: 207) | | | | 221) | | |

TABLE 2B
Exemplary heavy chain CDRs and FWRs of TRBC1-targeting
antigen binding domains derived from JOVI.1 shown in Table 2A
(according to ABM numbering convention)

| AB ID | VHFWR1 | VHCDR1 | VHFWR2 | VHCDR2 | VHFWR3 | VHCDR3 | VHFWR4 |
|---|---|---|---|---|---|---|---|
| mJOVI.1- | EVRLQQS | GYTFTG | WVKQRP | FINPYN | KATLTSD | GAGYNF | WGQGTT |
| H | GPDLIKP | YVMH | GQGLEWI | DDIQSN | KSSTTAY | DGAYRFF | LTVSS |
| | GASVKM | (SEQ ID | G (SEQ ID | ERFRG | MELSSLTS | DF (SEQ | (SEQ ID |
| | SCKAS | NO: | NO: 204) | (SEQ ID | EDSAVYY | ID NO: | NO: 206) |
| | (SEQ ID | 8401) | | NO: 201) | CAR (SEQ | 202) | |
| | NO: 8400) | | | | ID NO: | | |
| | | | | | 8679) | | |
| h1JOVI.1- | QVQLVQ | GYTFTG | WVRQAP | FINPYN | RVTMTSD | GAGYNF | WGQGTL |
| H | SGAEVK | YVMH | GQGLEW | DDIQSN | KSTTTAY | DGAYRFF | VTVSS |
| | KPGASVK | (SEQ ID | MG (SEQ | ERFRG | MELSSLRS | DF (SEQ | (SEQ ID |
| | VSCKAS | NO: | ID NO: | (SEQ ID | EDTAVYY | ID NO: | NO: 210) |
| | (SEQ ID | 8401) | 208) | NO: 201) | CAR (SEQ | 202) | |
| | NO: 8402) | | | | ID NO: | | |
| | | | | | 209) | | |
| h2JOVI.1- | QVQLVQ | GYTFTG | WVRQAP | FINPYN | WVTMTSD | GAGYNF | WGQGTL |
| H | SGAEVK | YVMH | GQGLEW | DDIQSN | KSITTAYM | DGAYRFF | VTVSS |
| | KPGASVK | (SEQ ID | MG (SEQ | ERFRG | ELSRLRSD | DF (SEQ | (SEQ ID |
| | VSCKAS | NO: | ID NO: | (SEQ ID | DTAVYYC | ID NO: | NO: 210) |
| | (SEQ ID | 8401) | 208) | NO: 201) | AR (SEQ | 202) | |
| | NO: 8403) | | | | ID NO: | | |
| | | | | | 213) | | |
| h3JOVI.1- | QVQLVQ | GYTFTG | WVRQAP | FINPYN | RVTITSDK | GAGYNF | WGQGTL |
| H | SGAEVK | YVMH | GQGLEW | DDIQSN | STTTAYM | DGAYRFF | VTVSS |
| | KPGSSVK | (SEQ ID | MG (SEQ | ERFRG | ELSSLRSE | DF (SEQ | (SEQ ID |
| | VSCKAS | NO: | ID NO: | (SEQ ID | DTAVYYC | ID NO: | NO: 210) |
| | (SEQ ID | 8401) | 208) | NO: 201) | AR (SEQ | 202) | |
| | NO: 8404) | | | | ID NO: | | |
| | | | | | 217) | | |
| h4JOVI.1- | QVQLVQ | GYTFTG | WVRQAP | FINPYN | RVTITSDK | GAGYNF | WGQGTL |
| H | SGAEVK | YVMH | GQRLEW | DDIQSN | SATTAYM | DGAYRFF | VTVSS |
| | KPGASVK | (SEQ ID | MG (SEQ | ERFRG | ELSSLRSE | DF (SEQ | (SEQ ID |
| | VSCKAS | NO: | ID NO: | (SEQ ID | DTAVYYC | ID NO: | NO: 210) |
| | (SEQ ID | 8401) | 212) | NO: 201) | AR (SEQ | 202) | |
| | NO: 8405) | | | | ID NO: | | |
| | | | | | 221) | | |

TABLE 3
Constant region amino acid sequences of human
IgG heavy chains and human kappa light chain.

| | | |
|---|---|---|
| Human kappa | LC | RTVAAPSVFI FPPSDEQLKS GTASVVCLLN NFYPREAKVQ WKVDNALQSG |
| constant region | | NSQESVTEQD SKDSTYSLSS TLTLSKADYE KHKVYACEVT HQGLSSPVTK |
| SEQ ID NO: 39 | | SFNRGEC |
| Human | LC | RTVAAPSVFIFPPSDEQLKSGTASVVCLLNNFYPREAKVQWKVDNALQSGNSQE |
| Immunoglobulin | | SVTEQDSKDSTYSLSSTLTLSKADYEKHKVYACEVTHQGLSSPVTKSFNRGEC |
| kappa constant C | | |
| region | | |
| SEQ ID NO: 3644 | | |
| IgG4 (S228P) | HC | ASTKGPSVFPLAPCSRSTSESTAALGCLVKDYFPEPVTVSWNSGALTSGVHTFP |
| mutant constant | | AVLQSSGLYSLSSVVTVPSSSLGTKTYTCNVDHKPSNTKVDKRVESKYGPPCPP |
| region (EU | | CPAPEFLGGPSVFLFPPKPKDTLMISRTPEVTCVVVDVSQEDPEVQFNWYVDGV |
| Numbering) | | EVHNAKTKPREEQFNSTYRVVSVLTVLHQDWLNGKEYKCKVSNKGLPSSIEKTI |
| SEQ ID NO: 40 | | SKAKGQPREPQVYTLPPSQEEMTKNQVSLTCLVKGFYPSDIAVEWESNGQPENN |
| | | YKTTPPVLDSDGSFFLYSRLTVDKSRWQEGNVFSCSVMHEALHNHYTQKSLSLS |
| | | LG |
| IgG1 wild type | HC | ASTKGPSVFPLAPSSKSTSGGTAALGCLVKDYFPEPVTVSWNSGALTSGVHTFP |
| SEQ ID NO: 41 | | AVLQSSGLYSLSSVVTVPSSSLGTQTYICNVNHKPSNTKVDKRVEPKSCDKTHT |
| | | CPPCPAPELLGGPSVFLFPPKPKDTLMISRTPEVTCVVVDVSHEDPEVKFNWYV |
| | | DGVEVHNAKTKPREEQYNSTYRVVSVLTVLHQDWLNGKEYKCKVSNKALPAPIE |
| | | KTISKAKGQPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALHNHYTQKSL |
| | | SLSPGK |
| IgG1 (N297A) | HC | ASTKGPSVFPLAPSSKSTSGGTAALGCLVKDYFPEPVTVSWNSGALTSGVHTFP |
| mutant constant | | AVLQSSGLYSLSSVVTVPSSSLGTQTYICNVNHKPSNTKVDKRVEPKSCDKTHT |
| region (EU | | CPPCPAPELLGGPSVFLFPPKPKDTLMISRTPEVTCVVVDVSHEDPEVKFNWYV |
| Numbering) | | DGVEVHNAKTKPREEQYASTYRVVSVLTVLHQDWLNGKEYKCKVSNKALPAPIE |
| SEQ ID NO: 42 | | KTISKAKGQPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALHNHYTQKSL |
| | | SLSPGK |
| IgM constant | HC | GSASAPTLFPLVSCENSPSDTSSVAVGCLAQDFLPDSITFSWKYKNNSDISSTR |
| delta CDC | | GFPSVLRGGKYAATSQVLLPSKDVMQGTDEHVVCKVQHPNGNKEKNVPLPVIAE |
| (P311A, P313S) | | LPPKVSVFVPPRDGFFGNPRKSKLICQATGFSPRQIQVSWLREGKQVGSGVTTD |
| SEQ ID NO: 73 | | QVQAEAKESGPTTYKVTSTLTIKESDWLGQSMFTCRVDHRGLTFQQNASSMCVP |
| | | DQDTAIRVFAIPPSFASIFLTKSTKLTCLVTDLTTYDSVTISWTRQNGEAVKTH |
| | | TNISESHPNATFSAVGEASICEDDWNSGERFTCTVTHTDLASSLKQTISRPKGV |
| | | ALHRPDVYLLPPAREQLNLRESATITCLVTGFSPADVFVQWMQRGQPLSPEKYV |
| | | TSAPMPEPQAPGRYFAHSILTVSEEEWNTGETYTCVVAHEALPNRVTERTVDKS |
| | | TGKPTLYNVSLVMSDTAGTCY |
| IgGA1 | HC | ASPTSPKVFPLSLCSTQPDGNVVIACLVQGFFPQEPLSVTWSESGQGVTARNFP |
| SEQ ID NO: 74 | | PSQDASGDLYTTSSQLTLPATQCLAGKSVTCHVKHYTNPSQDVTVPCPVPSTPP |
| | | TPSPSTPPTPSPSCCHPRLSLHRPALEDLLLGSEANLTCTLTGLRDASGVTFTW |
| | | TPSSGKSAVQGPPERDLCGCYSVSSVLPGCAEPWNHGKTFTCTAAYPESKTPLT |
| | | ATLSKSGNTFRPEVHLLPPPSEELALNELVTLTCLARGFSPKDVLVRWLQGSQE |
| | | LPREKYLTWASRQEPSQGTTTFAVTSILRVAAEDWKKGDTFSCMVGHEALPLAF |
| | | TQKTIDRLAGKPTHVNVSVVMAEVDGTCY |
| IgGA2 | HC | ASPTSPKVFPLSLDSTPQDGNVVVACLVQGFFPQEPLSVTWSESGQNVTARNFP |
| SEQ ID NO: 75 | | PSQDASGDLYTTSSQLTLPATQCPDGKSVTCHVKHYTNSSQDVTVPCRVPPPPP |
| | | CCHPRLSLHRPALEDLLLGSEANLTCTLTGLRDASGATFTWTPSSGKSAVQGPP |
| | | ERDLCGCYSVSSVLPGCAQPWNHGETFTCTAAHPELKTPLTANITKSGNTFRPE |
| | | VHLLPPPSEELALNELVTLTCLARGFSPKDVLVRWLQGSQELPREKYLTWASRQ |
| | | EPSQGTTTYAVTSILRVAAEDWKKGETFSCMVGHEALPLAFTQKTIDRMAGKPT |
| | | HINVSVVMAEADGTCY |
| Human Ig_J chain | HC | MKNHLLFWGVLAVFIKAVHVKAQEDERIVLVDNKCKCARITSRIIRSSEDPNED |
| SEQ ID NO: 76 | | IVERNIRIIVPLNNRENISDPTSPLRTRFVYHLSDLCKKCDPTEVELDNQIVTA |
| | | TQSNICDEDSATETCYTYDRNKCYTAVVPLVYGGETKMVETALTPDACYPD |
| Human | HC | ASTKGPSVFPLAPSSKSTSGGTAALGCLVKDYFPEPVTVSWNSGALTSGVHTFP |
| Immunoglobulin | | AVLQSSGLYSLSSVVTVPSSSLGTQTYICNVNHKPSNTKVDKKVEPKSCDKTHT |
| heavy chain | | CPPCPAPELLGGPSVFLFPPKPKDTLMISRTPEVTCVVVDVSHEDPEVKFNWYV |
| SEQ ID NO: 3645 | | DGVEVHNAKTKPREEQYASTYRVVSVLTVLHQDWLNGKEYKCKVSNKALPAPIE |
| | | KTISKAKGQPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALHNHYTQKSL |
| | | SLSPGK |
| Human | HC | ASTKGPSVFPLAPSSKSTSGGTAALGCLVKDYFPEPVTVSWNSGALTSGVHTFP |
| Immunoglobulin | | AVLQSSGLYSLSSVVTVPSSSLGTQTYICNVNHKPSNTKVDKKVEPKSCDKTHT |
| heavy chain | | CPPCPAPELLGGPSVFLFPPKPKDTLMISRTPEVTCVVVDVSHEDPEVKFNWYV |
| SEQ ID NO: 3646 | | DGVEVHNAKTKPREEQYASTYRVVSVLTVLHQDWLNGKEYKCKVSNKALPAPIE |
| | | KTISKAKGQPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALHNHYTQKSL |
| | | SLSPGKEPEA |
| Human | HC | ASTKGPSVFPLAPSSKSTSGGTAALGCLVKDYFPEPVTVSWNSGALTSGVHTFP |
| Immunoglobulin | | AVLQSSGLYSLSSVVTVPSSSLGTQTYICNVNHKPSNTKVDKRVEPKSCDKTHT |
| heavy chain | | CPPCPAPELLGGPSVFLFPPKPKDTLMISRTPEVTCVVVDVSHEDPEVKFNWYV |
| SEQ ID NO: 3647 | | DGVEVHNAKTKPREEQYNSTYRVVSVLTVLHQDWLNGKEYKCKVSNKALPAPIE |
| | | KTISKAKGQPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALHNHYTQKSL |
| | | SLSPGK |
| Human Fc Knob | HC | DKTHTCPPCPAPELLGGPSVFLFPPKPKDTLMISRTPEVTCVVVDVSHEDPEVK |
| cys N297A | | FNWYVDGVEVHNAKTKPREEQYASTYRVVSVLTVLHQDWINGKEYKCKVSNKAL |
| SEQ ID NO: 3648 | | PAPIEKTISKAKGQPREPQVYTLPPCREEMTKNQVSLWCLVKGFYPSDIAVEWE |
| | | SNGQPENNYKTTPPVLDSDGSFFLYSKLTVDKSRWQQGNVFSCSVMHEALHNHY |
| | | TQKSLSLSPGK |
| Human IgG1 hole | HC | ASTKGPSVFPLAPSSKSTSGGTAALGCLVKDYFPEPVTVSWNSGALTSGVHTFP |
| cys N297A | | AVLQSSGLYSLSSVVTVPSSSLGTQTYICNVNHKPSNTKVDKRVEPKSCDKTHT |
| SEQ ID NO: 3649 | | CPPCPAPELLGGPSVFLFPPKPKDTLMISRTPEVTCVVVDVSHEDPEVKFNWYV |
| | | DGVEVHNAKTKPREEQYASTYRVVSVLTVLHQDWLNGKEYKCKVSNKALPAPIE |
| | | KTISKAKGQPREPQVCTLPPSREEMTKNQVSLSCAVKGFYPSDIAVEWESNGQP |
| | | ENNYKTTPPVLDSDGSFFLVSKLTVDKSRWQQGNVFSCSVMHEALHNHYTQKSL |
| | | SLSPGK |

TABLE 3A
Exemplary heavy chain CDRs and FWRs of TRBC1-targeting
antigen binding domains derived from JOVI.1
(according to the Kabat numbering scheme)

| Ab ID | VHFWR1 | VHCDR1 | VHFWR2 | VHCDR2 | VHFWR3 | VHCDR3 | VHFWR4 |
|---|---|---|---|---|---|---|---|
| mJOVI.1- | EVRLQQS | GYVMH | WVKQR | FINPYND | KATLTSD | GAGYNF | WGQGTT |
| H | GPDLIKP | (SEQ ID | PGQGLE | DIQSNER | KSSTTAY | DGAYRFF | LTVSS |
| | GASVKM | NO: | WIG | FRG (SEQ | MELSSLTS | DF (SEQ | (SEQ ID |
| | SCKASGY | 8643) | (SEQ ID | ID NO: | EDSAVYY | ID NO: | NO: 206) |
| | TFT (SEQ | | NO: 204) | 201) | CAR (SEQ | 202) | |
| | ID NO: | | | | ID NO: | | |
| | 7370) | | | | 8679) | | |
| h1JOVI.1- | QVQLVQ | GYVMH | WVRQA | FINPYND | RVTMTSD | GAGYNF | WGQGTL |
| H | SGAEVK | (SEQ ID | PGQGLE | DIQSNER | KSTTTAY | DGAYRFF | VTVSS |
| | KPGASVK | NO: | WMG | FRG (SEQ | MELSSLRS | DF (SEQ | (SEQ ID |
| | VSCKASG | 8643) | (SEQ ID | ID NO: | EDTAVYY | ID NO: | NO: 210) |
| | YTFT | | NO: 208) | 201) | CAR (SEQ | 202) | |
| | (SEQ ID | | | | ID NO: | | |
| | NO: 207) | | | | 209) | | |
| h2JOVI.1- | QVQLVQ | GYVMH | WVRQA | FINPYND | WVTMTSD | GAGYNF | WGQGTL |
| H | SGAEVK | (SEQ ID | PGQGLE | DIQSNER | KSITTAYM | DGAYRFF | VTVSS |
| | KPGASVK | NO: | WMG | FRG (SEQ | ELSRLRSD | DF (SEQ | (SEQ ID |
| | VSCKASG | 8643) | (SEQ ID | ID NO: | DTAVYYC | ID NO: | NO: 210) |
| | YTFT | | NO: 208) | 201) | AR (SEQ | 202) | |
| | (SEQ ID | | | | ID NO: | | |
| | NO: 207) | | | | 213) | | |
| h3JOVI.1- | QVQLVQ | GYVMH | WVRQA | FINPYND | RVTITSDK | GAGYNF | WGQGTL |
| H | SGAEVK | (SEQ ID | PGQGLE | DIQSNER | STTTAYM | DGAYRFF | VTVSS |
| | KPGSSVK | NO: | WMG | FRG (SEQ | ELSSLRSE | DF (SEQ | (SEQ ID |
| | VSCKASG | 8643) | (SEQ ID | ID NO: | DTAVYYC | ID NO: | NO: 210) |
| | YTFT | | NO: 208) | 201) | AR (SEQ | 202) | |
| | (SEQ ID | | | | ID NO: | | |
| | NO: 7345) | | | | 217) | | |
| h4JOVI.1- | QVQLVQ | GYVMH | WVRQA | FINPYND | RVTITSDK | GAGYNF | WGQGTL |
| H | SGAEVK | (SEQ ID | PGQRLE | DIQSNER | SATTAYM | DGAYRFF | VTVSS |
| | KPGASVK | NO: | WMG | FRG (SEQ | ELSSLRSE | DF (SEQ | (SEQ ID |
| | VSCKASG | 8643) | (SEQ ID | ID NO: | DTAVYYC | ID NO: | NO: 210) |
| | YTFT | | NO: 212) | 201) | AR (SEQ | 202) | |
| | (SEQ ID | | | | ID NO: | | |
| | NO: 207) | | | | 221) | | |
| h5JOVI.1- | QVQLVQ | GYVMH | WVRQA | FINPYND | RVTITSDK | GAGYNF | WGQGTT |
| H | SGAEVK | (SEQ ID | PGQGLE | DIQSNER | STTTAYM | DGAYRFF | VTVSS |
| | KPGSSVK | NO: | WMG | FRG (SEQ | ELSSLRSE | DF (SEQ | (SEQ ID |
| | VSCKASG | 8643) | (SEQ ID | ID NO: | DTAVYYC | ID NO: | NO: 7347) |
| | YTFT | | NO: 208) | 201) | AR (SEQ | 202) | |
| | (SEQ ID | | | | ID NO: | | |
| | NO: 7345) | | | | 217) | | |
| h6JOVI.1- | QVQLVQ | GYVMH | WVRQA | FINPYND | RVTMTSD | GAGYNF | WGQGTT |
| H | SGAEVK | (SEQ ID | PGQGLE | DIQSNER | KSITTAYM | DGAYRFF | VTVSS |
| | KPGASVK | NO: | WMG | FRG (SEQ | ELSRLRSD | DF (SEQ | (SEQ ID |
| | VSCKASG | 8643) | (SEQ ID | ID NO: | DTAVYYC | ID NO: | NO: 7347) |
| | YTFT | | NO: 208) | 201) | AR (SEQ | 202) | |
| | (SEQ ID | | | | ID NO: | | |
| | NO: 207) | | | | 7349) | | |
| H1 | QVQLVQ | GYAIS | WVRQA | FINPYND | RVTITSDK | GAGYNF | WGQGTL |
| germlined- | SGAEVK | (SEQ ID | PGQGLE | DIQSNER | STTTAYM | DGAYRFF | VTVSS |
| VH | KPGSSVK | NO: | WMG | FRG (SEQ | ELSSLRSE | DF (SEQ | (SEQ ID |
| | VSCKASG | 7354) | (SEQ ID | ID NO: | DTAVYYC | ID NO: | NO: 210) |
| | YTFS | | NO: 208) | 201) | AR (SEQ | 202) | |
| | (SEQ ID | | | | ID NO: | | |
| | NO: 7353) | | | | 217) | | |
| H2 | QVQLVQ | GYVMH | WVRQA | FIIPIFGTA | RVTITSDK | GAGYNF | WGQGTL |
| germlined- | SGAEVK | (SEQ ID | PGQGLE | NYAQKF | STTTAYM | DGAYRFF | VTVSS |
| VH | KPGSSVK | NO: | WMG | QG (SEQ | ELSSLRSE | DF (SEQ | (SEQ ID |
| | VSCKASG | 8643) | (SEQ ID | ID NO: | DTAVYYC | ID NO: | NO: 210) |
| | YTFT | | NO: 208) | 7355) | AR (SEQ | 202) | |
| | (SEQ ID | | | | ID NO: | | |
| | NO: 7345) | | | | 217) | | |
| H1/H2 | QVQLVQ | GYAIS | WVRQA | FIIPIFGTA | RVTITSDK | GAGYNF | WGQGTL |
| germlined- | SGAEVK | (SEQ ID | PGQGLE | NYAQKF | STTTAYM | DGAYRFF | VTVSS |
| VH | KPGSSVK | NO: | WMG | QG (SEQ | ELSSLRSE | DF (SEQ | (SEQ ID |
| | VSCKASG | 7354) | (SEQ ID | ID NO: | DTAVYYC | ID NO: | NO: 210) |
| | YTFS | | NO: 208) | 7355) | AR (SEQ | 202) | |
| | (SEQ ID | | | | ID NO: | | |
| | NO: 7353) | | | | 217) | | |

TABLE 3B
Exemplary heavy chain CDRs and FWRs of TRBC1-targeting
antigen binding domains derived from JOVI.1 of Table 3A
(according to the ABM numbering scheme)

| Ab ID | VHFWR1 | VHCDR1 | VHFWR2 | VHCDR2 | VHFWR3 | VHCDR3 | VHFWR4 |
|---|---|---|---|---|---|---|---|
| mJOVI.1- | EVRLQQS | GYTFTG | WVKQR | FINPYND | KATLTSD | GAGYNF | WGQGTT |
| H | GPDLIKP | YVMH | PGQGLE | DIQSNER | KSSTTAY | DGAYRFF | LTVSS |
| | GASVKM | (SEQ ID | WIG | FRG (SEQ | MELSSLTS | DF (SEQ | (SEQ ID |
| | SCKAS | NO: | (SEQ ID | ID NO: | EDSAVYY | ID NO: | NO: 206) |
| | (SEQ ID | 8507) | NO: 204) | 201) | CAR (SEQ | 202) | |
| | NO: 8506) | | | | ID NO: | | |
| | | | | | 8679) | | |
| h1JOVI.1- | QVQLVQ | GYTFTG | WVRQA | FINPYND | RVTMTSD | GAGYNF | WGQGTL |
| H | SGAEVK | YVMH | PGQGLE | DIQSNER | KSTTTAY | DGAYRFF | VTVSS |
| | KPGASVK | (SEQ ID | WMG | FRG (SEQ | MELSSLRS | DF (SEQ | (SEQ ID |
| | VSCKAS | NO: | (SEQ ID | ID NO: | EDTAVYY | ID NO: | NO: 210) |
| | (SEQ ID | 8507) | NO: 208) | 201) | CAR (SEQ | 202) | |
| | NO: 8508) | | | | ID NO: | | |
| | | | | | 209) | | |
| h2JOVI.1- | QVQLVQ | GYTFTG | WVRQA | FINPYND | WVTMTSD | GAGYNF | WGQGTL |
| H | SGAEVK | YVMH | PGQGLE | DIQSNER | KSITTAYM | DGAYRFF | VTVSS |
| | KPGASVK | (SEQ ID | WMG | FRG (SEQ | ELSRLRSD | DF (SEQ | (SEQ ID |
| | VSCKAS | NO: | (SEQ ID | ID NO: | DTAVYYC | ID NO: | NO: 210) |
| | (SEQ ID | 8507) | NO: 208) | 201) | AR (SEQ | 202) | |
| | NO: 8508) | | | | ID NO: | | |
| | | | | | 213) | | |
| h3JOVI.1- | QVQLVQ | GYTFT | WVRQA | FINPYND | RVTITSDK | GAGYNF | WGQGTL |
| H | SGAEVK | GYVMH | PGQGLE | DIQSNER | STTTAYM | DGAYRFF | VTVSS |
| | KPGSSVK | (SEQ ID | WMG | FRG (SEQ | ELSSLRSE | DF (SEQ | (SEQ ID |
| | VSCKAS | NO: | (SEQ ID | ID NO: | DTAVYYC | ID NO: | NO: 210) |
| | (SEQ ID | 8507) | NO: 208) | 201) | AR (SEQ | 202) | |
| | NO: 8509) | | | | ID NO: | | |
| | | | | | 217) | | |
| h4JOVI.1- | QVQLVQ | GYTFTG | WVRQA | FINPYND | RVTITSDK | GAGYNF | WGQGTL |
| H | SGAEVK | YVMH | PGQRLE | DIQSNER | SATTAYM | DGAYRFF | VTVSS |
| | KPGASVK | (SEQ ID | WMG | FRG (SEQ | ELSSLRSE | DF (SEQ | (SEQ ID |
| | VSCKAS | NO: | (SEQ ID | ID NO: | DTAVYYC | ID NO: | NO: 210) |
| | (SEQ ID | 8507) | NO: 212) | 201) | AR (SEQ | 202) | |
| | NO: 8508) | | | | ID NO: | | |
| | | | | | 221) | | |
| h5JOVI.1- | QVQLVQ | GYTFTG | WVRQA | FINPYND | RVTITSDK | GAGYNF | WGQGTT |
| H | SGAEVK | YVMH | PGQGLE | DIQSNER | STTTAYM | DGAYRFF | VTVSS |
| | KPGSSVK | (SEQ ID | WMG | FRG (SEQ | ELSSLRSE | DF (SEQ | (SEQ ID |
| | VSCKAS | NO: | (SEQ ID | ID NO: | DTAVYYC | ID NO: | NO: 7347) |
| | (SEQ ID | 8507) | NO: 208) | 201) | AR (SEQ | 202) | |
| | NO: 8510) | | | | ID NO: | | |
| | | | | | 217) | | |
| h6JOVI.1- | QVQLVQ | GYTFTG | WVRQA | FINPYND | RVTMTSD | GAGYNF | WGQGTT |
| H | SGAEVK | YVMH | PGQGLE | DIQSNER | KSITTAYM | DGAYRFF | VTVSS |
| | KPGASVK | (SEQ ID | WMG | FRG (SEQ | ELSRLRSD | DF (SEQ | (SEQ ID |
| | VSCKAS | NO: | (SEQ ID | ID NO: | DTAVYYC | ID NO: | NO: 7347) |
| | (SEQ ID | 8507) | NO: 208) | 201) | AR (SEQ | 202) | |
| | NO: 8511) | | | | ID NO: | | |
| | | | | | 7349) | | |
| H1 | QVQLVQ | GYTFSG | WVRQA | FINPYND | RVTITSDK | GAGYNF | WGQGTL |
| germlined- | SGAEVK | YAIS | PGQGLE | DIQSNER | STTTAYM | DGAYRFF | VTVSS |
| VH | KPGSSVK | (SEQ ID | WMG | FRG (SEQ | ELSSLRSE | DF (SEQ | (SEQ ID |
| | VSCKAS | NO: | (SEQ ID | ID NO: | DTAVYYC | ID NO: | NO: 210) |
| | (SEQ ID | 8513) | NO: 208) | 201) | AR (SEQ | 202) | |
| | NO: 8512) | | | | ID NO: | | |
| | | | | | 217) | | |
| H2 | QVQLVQ | GYTFTG | WVRQA | FIIPIFGTA | RVTITSDK | GAGYNF | WGQGTL |
| germlined- | SGAEVK | YVMH | PGQGLE | NYAQKF | STTTAYM | DGAYRFF | VTVSS |
| VH | KPGSSVK | (SEQ ID | WMG | QG (SEQ | ELSSLRSE | DF (SEQ | (SEQ ID |
| | VSCKAS | NO: | (SEQ ID | ID NO: | DTAVYYC | ID NO: | NO: 210) |
| | (SEQ ID | 8507) | NO: 208) | 7355) | AR (SEQ | 202) | |
| | NO: 8514) | | | | ID NO: | | |
| | | | | | 217) | | |
| H1/H2 | QVQLVQ | GYTFSG | WVRQA | FIIPIFGTA | RVTITSDK | GAGYNF | WGQGTL |
| germlined- | SGAEVK | YAIS | PGQGLE | NYAQKF | STTTAYM | DGAYRFF | VTVSS |
| VH | KPGSSVK | (SEQ ID | WMG | QG (SEQ | ELSSLRSE | DF (SEQ | (SEQ ID |
| | VSCKAS | NO: | (SEQ ID | ID NO: | DTAVYYC | ID NO: | NO: 210) |
| | (SEQ ID | 8513) | NO: 208) | 7355) | AR (SEQ | 202) | |
| | NO: 8515) | | | | ID NO: | | |
| | | | | | 217) | | |

TABLE 4
Exemplary light chain CDRs and FWRs of TRBC1-targeting
antigen binding domains derived from JOVI.1
(According to Kabat numbering scheme)

| Ab ID | VLFWR1 | VLCDR1 | VLFWR2 | VLCDR2 | VLFWR3 | VLCDR3 | VLFWR4 |
|---|---|---|---|---|---|---|---|
| mJOVI.1- | DVVMTQ | RSSQRL | WYLQKP | RVSNRFP | GVPDRFS | SQSTHVP | FGGGTKL |
| L | SPLSLPVS | VHSNGN | GQSPKL | (SEQ ID | GSGSGTDF | YT (SEQ | EIK (SEQ |
| | LGDQASI | TYLH | LIY (SEQ | NO: 224) | TLKISRVE | ID NO: | ID NO: |
| | SC (SEQ | (SEQ ID | ID NO: | | AEDLGIYF | 225) | 229) |
| | ID NO: | NO: 223) | 227) | | C (SEQ ID | | |
| | 226) | | | | NO: 228) | | |
| h1JOVI.1- | DVVMTQ | RSSQRL | WYLQKP | RVSNRFP | GVPDRFS | SQSTHVP | FGGGTK |
| L | SPLSLPV | VHSNGN | GQSPQL | (SEQ ID | GSGSGTDF | YT (SEQ | VEIK |
| | TPGEPASI | TYLH | LIY (SEQ | NO: 224) | TLKISRVE | ID NO: | (SEQ ID |
| | SC (SEQ | (SEQ ID | ID NO: | | AEDVGVY | 225) | NO: 8685) |
| | ID NO: | NO: 223) | 231) | | FC (SEQ ID | | |
| | 230) | | | | NO: 240) | | |
| h2JOVI.1- | EVVMTQ | RSSQRL | WYQQK | RVSNRFP | GIPDRFSG | SQSTHVP | FGGGTK |
| L | SPGTLSL | VHSNGN | PGQAPR | (SEQ ID | SGSGTDFT | YT (SEQ | VEIK |
| | SPGERAT | TYLH | LLIY | NO: 224) | LTISRLEPE | ID NO: | (SEQ ID |
| | LSC (SEQ | (SEQ ID | (SEQ ID | | DFAVYFC | 225) | NO: 237) |
| | ID NO: | NO: 223) | NO: 235) | | (SEQ ID | | |
| | 234) | | | | NO: 236) | | |
| h3JOVI.1- | DVVMTQ | RSSQRL | WYQQR | RVSNRFP | GVPDRFS | SQSTHVP | FGGGTK |
| L | SPLSLPV | VHSNGN | PGQSPR | (SEQ ID | GSGSGTDF | YT (SEQ | VEIK |
| | TLGQPAS | TYLH | LLIY | NO: 224) | TLKISRVE | ID NO: | (SEQ ID |
| | ISC (SEQ | (SEQ ID | (SEQ ID | | AEDVGVY | 225) | NO: 241) |
| | ID NO: | NO: 223) | NO: 239) | | FC (SEQ ID | | |
| | 238) | | | | NO: 240) | | |
| h4JOVI.1- | DVVMTQ | RSSQRL | WYLQKP | RVSNRFP | GVPDRFS | SQSTHVP | FGGGTK |
| L | TPLSLPV | VHSNGN | GQSPQL | (SEQ ID | GSGSGTDF | YT (SEQ | VEIK |
| | TPGEPASI | TYLH | LIY (SEQ | NO: 224) | TLKISRVE | ID NO: | (SEQ ID |
| | SC (SEQ | (SEQ ID | ID NO: | | AEDVGVY | 225) | NO: 245) |
| | ID NO: | NO: 223) | 243) | | FC (SEQ ID | | |
| | 242) | | | | NO: 240) | | |
| h5JOVI.1- | DVVMTQ | RSSQRL | WYLQKP | RVSNRFP | GVPDRFS | SQSTHVP | FGGGTK |
| L | TPLSLSV | VHSNGN | GQSPQL | (SEQ ID | GSGSGTDF | YT (SEQ | VEIK |
| | TPGQPAS | TYLH | LIY (SEQ | NO: 224) | TLKISRVE | ID NO: | (SEQ ID |
| | ISC (SEQ | (SEQ ID | ID NO: | | AEDVGVY | 225) | NO: 249) |
| | ID NO: | NO: 223) | 247) | | FC (SEQ ID | | |
| | 246) | | | | NO: 240) | | |
| L1 | DVVMTQ | RSSQSL | WYQQR | RVSNRFP | GVPDRFS | SQSTHVP | FGGGTK |
| germlined- | SPLSLPV | VYSDGN | PGQSPR | (SEQ ID | GSGSGTDF | YT (SEQ | VEIK |
| VL | TLGQPAS | TYH | LLIY | NO: 224) | TLKISRVE | ID NO: | (SEQ ID |
| | ISC (SEQ | (SEQ ID | (SEQ ID | | AEDVGVY | 225) | NO: 8685) |
| | ID NO: | NO: | NO: 239) | | FC (SEQ ID | | |
| | 238) | 7367) | | | NO: 240) | | |
| L2 | DVVMTQ | RSSQRL | WYQQR | KVSNRDS | GVPDRFS | SQSTHVP | FGGGTK |
| germlined- | SPLSLPV | VHSNGN | PGQSPR | (SEQ ID | GSGSGTDF | YT (SEQ | VEIK |
| VL | TLGQPAS | TYLH | LLIY | NO: 7368) | TLKISRVE | ID NO: | (SEQ ID |
| | ISC (SEQ | (SEQ ID | (SEQ ID | | AEDVGVY | 225) | NO: 8685) |
| | ID NO: | NO: 223) | NO: 239) | | FC (SEQ ID | | |
| | 238) | | | | NO: 240) | | |
| L3 | DVVMTQ | RSSQRL | WYQQR | RVSNRFP | GVPDRFS | MQSTHW | FGGGTK |
| germlined- | SPLSLPV | VHSNGN | PGQSPR | (SEQ ID | GSGSGTDF | PYT (SEQ | VEIK |
| VL | TLGQPAS | TYLH | LLIY | NO: 224) | TLKISRVE | ID NO: | (SEQ ID |
| | ISC (SEQ | (SEQ ID | (SEQ ID | | AEDVGVY | 7369) | NO: 8685) |
| | ID NO: | NO: 223) | NO: 239) | | FC (SEQ ID | | |
| | 238) | | | | NO: 240) | | |
| L1/L2/L3 | DVVMTQ | RSSQSL | WYQQR | KVSNRDS | GVPDRFS | MQSTHW | FGGGTK |
| germlined- | SPLSLPV | VYSDGN | PGQSPR | (SEQ ID | GSGSGTDF | PYT (SEQ | VEIK |
| VL | TLGQPAS | TYH | LLIY | NO: 7368) | TLKISRVE | ID NO: | (SEQ ID |
| | ISC (SEQ | (SEQ ID | (SEQ ID | | AEDVGVY | 7369) | NO: 8685) |
| | ID NO: | NO: | NO: 239) | | FC (SEQ ID | | |
| | 238) | 7367) | | | NO: 240) | | |

TABLE 5A
Exemplary heavy chain CDRs and FWRs of
TRBC1-targeting antigen binding domains
(According to Kabat numbering scheme)

| Ab ID | VHFWR1 | VHCDR1 | VHFWR2 | VHCDR2 | VHFWR3 | VHCDR3 | VHFWR4 |
|---|---|---|---|---|---|---|---|
| BKM0191 | QVQLVQ | GYVMH | WVRQA | FIIPIFGTA | RVTITSDK | GAGYNFD | WGQGT |
| | SGAEVK | (SEQ ID | PGQGLE | NYAQKF | STTTAYM | GAYRFFD | LVTVSS |
| | KPGSSVK | NO: | WMG | QG (SEQ | ELSSLRSE | F (SEQ ID | (SEQ ID |
| | VSCKASG | 8643) | (SEQ ID | ID NO: | DTAVYYC | NO: 202) | NO: 210) |
| | YTFT | | NO: 208) | 7355) | AR (SEQ | | |
| | (SEQ ID | | | | ID NO: | | |
| | NO: 7345) | | | | 217) | | |
| BKM0192 | QVQLVQ | GYVMH | WVRQA | FIIPIFGTA | RVTITSDK | GAGYNFD | WGQGT |
| | SGAEVK | (SEQ ID | PGQGLE | NYAQKF | STTTAYM | GAYRFFD | LVTVSS |
| | KPGSSVK | NO: | WMG | QG (SEQ | ELSSLRSE | F (SEQ ID | (SEQ ID |
| | VSCKASG | 8643) | (SEQ ID | ID NO: | DTAVYYC | NO: 202) | NO: 210) |
| | YTFT | | NO: 208) | 7355) | AR (SEQ | | |
| | (SEQ ID | | | | ID NO: | | |
| | NO: 7345) | | | | 217) | | |
| BKM0193 | QVQLVQ | GYVMH | WVRQA | FIIPIFGTA | RVTITSDK | GAGYNFD | WGQGT |
| | SGAEVK | (SEQ ID | PGQGLE | NYAQKF | STTTAYM | GAYRFFD | LVTVSS |
| | KPGSSVK | NO: | WMG | QG (SEQ | ELSSLRSE | F (SEQ ID | (SEQ ID |
| | VSCKASG | 8643) | (SEQ ID | ID NO: | DTAVYYC | NO: 202) | NO: 210) |
| | YTFT | | NO: 208) | 7355) | AR (SEQ | | |
| | (SEQ ID | | | | ID NO: | | |
| | NO: 7345) | | | | 217) | | |
| BKM0194 | QVQLVQ | GYVMH | WVRQA | FIIPIFGTA | RVTITSDK | GAGYNFD | WGQGT |
| | SGAEVK | (SEQ ID | PGQGLE | NYAQKF | STTTAYM | GAYRFFD | LVTVSS |
| | KPGSSVK | NO: | WMG | QG (SEQ | ELSSLRSE | F (SEQ ID | (SEQ ID |
| | VSCKASG | 8643) | (SEQ ID | ID NO: | DTAVYYC | NO: 202) | NO: 210) |
| | YTFT | | NO: 208) | 7355) | AR (SEQ | | |
| | (SEQ ID | | | | ID NO: | | |
| | NO: 7345) | | | | 217) | | |
| BKM0195 | QVQLVQ | GYVMH | WVRQA | FIIPIFGTA | RVTITSDK | GAGYNFD | WGQGT |
| | SGAEVK | (SEQ ID | PGQGLE | NYAQKF | STTTAYM | GAYRFFD | LVTVSS |
| | KPGSSVK | NO: | WMG | QG (SEQ | ELSSLRSE | F (SEQ ID | (SEQ ID |
| | VSCKASG | 8643) | (SEQ ID | ID NO: | DTAVYYC | NO: 202) | NO: 210) |
| | YTFT | | NO: 208) | 7355) | AR (SEQ | | |
| | (SEQ ID | | | | ID NO: | | |
| | NO: 7345) | | | | 217) | | |
| BKM0196 | QVQLVQ | GYVMH | WVRQA | FIIPIFGTA | RVTITSDK | GAGYNFD | WGQGT |
| | SGAEVK | (SEQ ID | PGQGLE | NYAQKF | STTTAYM | GAYRFFD | LVTVSS |
| | KPGSSVK | NO: | WMG | QG (SEQ | ELSSLRSE | F (SEQ ID | (SEQ ID |
| | VSCKASG | 8643) | (SEQ ID | ID NO: | DTAVYYC | NO: 202) | NO: 210) |
| | YTFT | | NO: 208) | 7355) | AR (SEQ | | |
| | (SEQ ID | | | | ID NO: | | |
| | NO: 7345) | | | | 217) | | |

TABLE 5B
Exemplary heavy chain CDRs and FWRs of TRBC1-
targeting antigen binding domains of Table 5A
(According to ABM numbering scheme)

| Ab ID | VHFWR1 | VHCDR1 | VHFWR2 | VHCDR2 | VHFWR3 | VHCDR3 | VHFWR4 |
|---|---|---|---|---|---|---|---|
| BKM0191 | QVQLVQ | GYTFTG | WVRQA | FIIPIFGTA | RVTITSDK | GAGYNFD | WGQGT |
| | SGAEVK | YVMH | PGQGLE | NYAQKF | STTTAYM | GAYRFFD | LVTVSS |
| | KPGSSVK | (SEQ ID | WMG | QG (SEQ | ELSSLRSE | F (SEQ ID | (SEQ ID |
| | VSCKAS | NO: | (SEQ ID | ID NO: | DTAVYYC | NO: 202) | NO: 210) |
| | (SEQ ID | 8517) | NO: 208) | 7355) | AR (SEQ | | |
| | NO: 8516) | | | | ID NO: | | |
| | | | | | 217) | | |
| BKM0192 | QVQLVQ | GYTFTG | WVRQA | FIIPIFGTA | RVTITSDK | GAGYNFD | WGQGT |
| | SGAEVK | YVMH | PGQGLE | NYAQKF | STTTAYM | GAYRFFD | LVTVSS |
| | KPGSSVK | (SEQ ID | WMG | QG (SEQ | ELSSLRSE | F (SEQ ID | (SEQ ID |
| | VSCKAS | NO: | (SEQ ID | ID NO: | DTAVYYC | NO: 202) | NO: 210) |
| | (SEQ ID | 8519) | NO: 208) | 7355) | AR (SEQ | | |
| | NO: 8518) | | | | ID NO: | | |
| | | | | | 217) | | |
| BKM0193 | QVQLVQ | GYTFTG | WVRQA | FIIPIFGTA | RVTITSDK | GAGYNFD | WGQGT |
| | SGAEVK | YVMH | PGQGLE | NYAQKF | STTTAYM | GAYRFFD | LVTVSS |
| | KPGSSVK | (SEQ ID | WMG | QG (SEQ | ELSSLRSE | F (SEQ ID | (SEQ ID |
| | VSCKAS | NO: | (SEQ ID | ID NO: | DTAVYYC | NO: 202) | NO: 210) |
| | (SEQ ID | 8521) | NO: 208) | 7355) | AR (SEQ | | |
| | NO: 8520) | | | | ID NO: | | |
| | | | | | 217) | | |
| BKM0194 | QVQLVQ | GYTFTG | WVRQA | FIIPIFGTA | RVTITSDK | GAGYNFD | WGQGT |
| | SGAEVK | YVMH | PGQGLE | NYAQKF | STTTAYM | GAYRFFD | LVTVSS |
| | KPGSSVK | (SEQ ID | WMG | QG (SEQ | ELSSLRSE | F (SEQ ID | (SEQ ID |
| | VSCKAS | NO: | (SEQ ID | ID NO: | DTAVYYC | NO: 202) | NO: 210) |
| | (SEQ ID | 8523) | NO: 208) | 7355) | AR (SEQ | | |
| | NO: 8522) | | | | ID NO: | | |
| | | | | | 217) | | |
| BKM0195 | QVQLVQ | GYTFTG | WVRQA | FIIPIFGTA | RVTITSDK | GAGYNFD | WGQGT |
| | SGAEVK | YVMH | PGQGLE | NYAQKF | STTTAYM | GAYRFFD | LVTVSS |
| | KPGSSVK | (SEQ ID | WMG | QG (SEQ | ELSSLRSE | F (SEQ ID | (SEQ ID |
| | VSCKAS | NO: | (SEQ ID | ID NO: | DTAVYYC | NO: 202) | NO: 210) |
| | (SEQ ID | 8525) | NO: 208) | 7355) | AR (SEQ | | |
| | NO: 8524) | | | | ID NO: | | |
| | | | | | 217) | | |
| BKM0196 | QVQLVQ | GYTFTG | WVRQA | FIIPIFGTA | RVTITSDK | GAGYNFD | WGQGT |
| | SGAEVK | YVMH | PGQGLE | NYAQKF | STTTAYM | GAYRFFD | LVTVSS |
| | KPGSSVK | (SEQ ID | WMG | QG (SEQ | ELSSLRSE | F (SEQ ID | (SEQ ID |
| | VSCKAS | NO: | (SEQ ID | ID NO: | DTAVYYC | NO: 202) | NO: 210) |
| | (SEQ ID | 8527) | NO: 208) | 7355) | AR (SEQ | | |
| | NO: 8526) | | | | ID NO: | | |
| | | | | | 217) | | |

TABLE 6
Exemplary light chain CDRs and FWRs
of TRBC1-targeting antigen binding domains
(According to Kabat numbering scheme)

| Ab ID | VLFWR1 | VLCDR1 | VLFWR2 | VLCDR2 | VLFWR3 | VLCDR3 | VLFWR4 |
|---|---|---|---|---|---|---|---|
| BKM0191 | DVVMTQ | RSSQRL | WYQQR | RVSNRFP | GVPDRFS | SQSTHVPY | GGGTKV |
| | SPLSLPV | VHSNAN | PGQSPR | (SEQ ID | GSGSGTDF | TF (SEQ ID | EIK (SEQ |
| | TLGQPAS | TYLH | LLIY | NO: 224) | TLKISRVE | NO: 8680) | ID NO: |
| | ISC (SEQ | (SEQ ID | (SEQ ID | | AEDVGVY | | 373) |
| | ID NO: | NO: 367) | NO: 239) | | FC (SEQ ID | | |
| | 238) | | | | NO: 240) | | |
| BKM0192 | DVVMTQ | RSSQRL | WYQQR | RVSNRFP | GVPDRFS | SQSTHVPY | GGGTKV |
| | SPLSLPV | VHSNGN | PGQSPR | (SEQ ID | GSGSGTDF | TF (SEQ ID | EIK (SEQ |
| | TLGQPAS | AYLH | LLIY | NO: 224) | TLKISRVE | NO: 8680) | ID NO: |
| | ISC (SEQ | (SEQ ID | (SEQ ID | | AEDVGVY | | 373) |
| | ID NO: | NO: 368) | NO: 239) | | FC (SEQ ID | | |
| | 238) | | | | NO: 240) | | |
| BKM0193 | DVVMTQ | RSSQRL | WYQQR | RVSNRFP | GVPDRFS | SQSTHVPY | FGGGTK |
| | SPLSLPV | VHSNAN | PGQSPR | (SEQ ID | GSGSGTDF | T (SEQ ID | VEIK |
| | TLGQPAS | AYLH | LLIY | NO: 224) | TLKISRVE | NO: 225) | (SEQ ID |
| | ISC (SEQ | (SEQ ID | (SEQ ID | | AEDVGVY | | NO: |
| | ID NO: | NO: 369) | NO: 239) | | FC (SEQ ID | | 8685) |
| | 238) | | | | NO: 240) | | |
| BKM0194 | DVVMTQ | RSSQRL | WYQQR | RVSNRFP | GVPDRFS | SQSTHVPY | FGGGTK |
| | SPLSLPV | VHSGGN | PGQSPR | (SEQ ID | GSGSGTDF | T (SEQ ID | VEIK |
| | TLGQPAS | TYLH | LLIY | NO: 224) | TLKISRVE | NO: 225) | (SEQ ID |
| | ISC (SEQ | (SEQ ID | (SEQ ID | | AEDVGVY | | NO: |
| | ID NO: | NO: 370) | NO: 239) | | FC (SEQ ID | | 8685) |
| | 238) | | | | NO: 240) | | |
| BKM0195 | DVVMTQ | RSSQRL | WYQQR | RVSNRFP | GVPDRFS | SQSTHVPY | FGGGTK |
| | SPLSLPV | VHSNGS | PGQSPR | (SEQ ID | GSGSGTDF | T (SEQ ID | VEIK |
| | TLGQPAS | TYLH | LLIY | NO: 224) | TLKISRVE | NO: 225) | (SEQ ID |
| | ISC (SEQ | (SEQ ID | (SEQ ID | | AEDVGVY | | NO: |
| | ID NO: | NO: 371) | NO: 239) | | FC (SEQ ID | | 8685) |
| | 238) | | | | NO: 240) | | |
| BKM0196 | DVVMTQ | RSSQRL | WYQQR | RVSNRFP | GVPDRFS | SQSTHVPY | FGGGTK |
| | SPLSLPV | VHSGGS | PGQSPR | (SEQ ID | GSGSGTDF | T (SEQ ID | VEIK |
| | TLGQPAS | TYLH | LLIY | NO: 224) | TLKISRVE | NO: 225) | (SEQ ID |
| | ISC (SEQ | (SEQ ID | (SEQ ID | | AEDVGVY | | NO: |
| | ID NO: | NO: 372) | NO: 239) | | FC (SEQ ID | | 8685) |
| | 238) | | | | NO: 240) | | |

TABLE 7
Exemplary variable regions of TRBC1-targeting antigen binding domains

| SEQ ID | | | |
|---|---|---|---|
| NO | Ab ID | Description | Sequence |
| SEQ ID | mJOVI.1- | JOVI.1 heavy | EVRLQQSGPDLIKPGASVKMSCKASGYTFTGYVMHWVKQ |
| NO: 250 | H | chain variable | RPGQGLEWIGFINPYNDDIQSNERFRGKATLTSDKSSTT |
| | | region | AYMELSSLTSEDSAVYYCARGAGYNFDGAYRFFDFWGQG |
| | | | TTLTVSS |
| SEQ ID | h1JOVI.1- | JOVI.1 heavy | QVQLVQSGAEVKKPGASVKVSCKASGYTFTGYVMHWVRQ |
| NO: 251 | H | chain variable | APGQGLEWMGFINPYNDDIQSNERFRGRVTMTSDKSTTT |
| | | region | AYMELSSLRSEDTAVYYCARGAGYNFDGAYRFFDFWGQG |
| | | humanized | TLVTVSS |
| | | variant 1 | |
| SEQ ID | h2JOVI.1- | JOVI.1 heavy | QVQLVQSGAEVKKPGASVKVSCKASGYTFTGYVMHWVRQ |
| NO: 252 | H | chain variable | APGQGLEWMGFINPYNDDIQSNERFRGWVTMTSDKSITT |
| | | region | AYMELSRLRSDDTAVYYCARGAGYNFDGAYRFFDFWGQG |
| | | humanized | TLVTVSS |
| | | variant 2 | |
| SEQ ID | h3JOVI.1- | JOVI.1 heavy | QVQLVQSGAEVKKPGSSVKVSCKASGYTFTGYVMHWVRQ |
| NO: 253 | H | chain variable | APGQGLEWMGFINPYNDDIQSNERFRGRVTITSDKSTTT |
| | | region | AYMELSSLRSEDTAVYYCARGAGYNFDGAYRFFDFWGQG |
| | | humanized | TLVTVSS |
| | | variant 3 | |
| SEQ ID | h4JOVI.1- | JOVI.1 heavy | QVQLVQSGAEVKKPGASVKVSCKASGYTFTGYVMHWVRQ |
| NO: 254 | H | chain variable | APGQRLEWMGFINPYNDDIQSNERFRGRVTITSDKSATT |
| | | region | AYMELSSLRSEDTAVYYCARGAGYNFDGAYRFFDFWGQG |
| | | humanized | TLVTVSS |
| | | variant 4 | |
| SEQ ID | h5JOVI.1- | JOVI.1 heavy | QVQLVQSGAEVKKPGSSVKVSCKASGYTFTGYVMHWVRQ |
| NO: | H | chain variable | APGQGLEWMGFINPYNDDIQSNERFRGRVTITSDKSTTT |
| 7343 | | region | AYMELSSLRSEDTAVYYCARGAGYNFDGAYRFFDFWGQG |
| | | humanized | TTVTVSS |
| | | variant 5 | |
| SEQ ID | h6JOVI.1- | JOVI.1 heavy | QVQLVQSGAEVKKPGASVKVSCKASGYTFTGYVMHWVRQ |
| NO: | H | chain variable | APGQGLEWMGFINPYNDDIQSNERFRGRVTMTSDKSITT |
| 7344 | | region | AYMELSRLRSDDTAVYYCARGAGYNFDGAYRFFDFWGQG |
| | | humanized | TTVTVSS |
| | | variant 6 | |
| SEQ ID | H1 | JOVI.1 heavy | QVQLVQSGAEVKKPGSSVKVSCKASGYTFSGYAISWVRQ |
| NO: | germlined- | chain variable | APGQGLEWMGFINPYNDDIQSNERFRGRVTITSDKSTTT |
| 7350 | VH | region | AYMELSSLRSEDTAVYYCARGAGYNFDGAYRFFDFWGQG |
| | | humanized H1 | TLVTVSS |
| SEQ ID | H2 | JOVI.1 heavy | QVQLVQSGAEVKKPGSSVKVSCKASGYTFTGYVMHWVRQ |
| NO: | germlined- | chain variable | APGQGLEWMGFIIPIFGTANYAQKFQGRVTITSDKSTTT |
| 7351 | VH | region | AYMELSSLRSEDTAVYYCARGAGYNFDGAYRFFDFWGQG |
| | | humanized H2 | TLVTVSS |
| SEQ ID | H1/H2 | JOVI.1 heavy | QVQLVQSGAEVKKPGSSVKVSCKASGYTFSGYAISWVRQ |
| NO: | germlined- | chain variable | APGQGLEWMGFIIPIFGTANYAQKFQGRVTITSDKSTTT |
| 7352 | VH | region | AYMELSSLRSEDTAVYYCARGAGYNFDGAYRFFDFWGQG |
| | | humanized | TLVTVSS |
| | | H1/H2 | |
| SEQ ID | mJOVI.1- | JOVI.1 light | DVVMTQSPLSLPVSLGDQASISCRSSQRLVHSNGNTYLH |
| NO: 255 | L | chain variable | WYLQKPGQSPKLLIYRVSNRFPGVPDRFSGSGSGTDFTL |
| | | region | KISRVEAEDLGIYFCSQSTHVPYTFGGGTKLEIK |
| SEQ ID | BKM0191 | BKM0191 | QVQLVQSGAEVKKPGSSVKVSCKASGYTFTGYVMHWVRQ |
| NO: 517 | VH | G29A heavy | APGQGLEWMGFIIPIFGTANYAQKFQGRVTITSDKSTTT |
| | | chain variable | AYMELSSLRSEDTAVYYCARGAGYNFDGAYRFFDFWGQG |
| | | region | TLVTVSS |
| SEQ ID | BKM0192 | BKM0192 | QVQLVQSGAEVKKPGSSVKVSCKASGYTFTGYVMHWVRQ |
| NO: 517 | VH | T31A heavy | APGQGLEWMGFIIPIFGTANYAQKFQGRVTITSDKSTTT |
| | | chain variable | AYMELSSLRSEDTAVYYCARGAGYNFDGAYRFFDFWGQG |
| | | region | TLVTVSS |
| SEQ ID | BKM0193 | BKM0193 | QVQLVQSGAEVKKPGSSVKVSCKASGYTFTGYVMHWVRQ |
| NO: 517 | VH | G29a T31A | APGQGLEWMGFIIPIFGTANYAQKFQGRVTITSDKSTTT |
| | | heavy chain | AYMELSSLRSEDTAVYYCARGAGYNFDGAYRFFDFWGQG |
| | | variable region | TLVTVSS |
| SEQ ID | BKM0194 | BKM0194 | QVQLVQSGAEVKKPGSSVKVSCKASGYTFTGYVMHWVRQ |
| NO: 517 | VH | N28G heavy | APGQGLEWMGFIIPIFGTANYAQKFQGRVTITSDKSTTT |
| | | chain variable | AYMELSSLRSEDTAVYYCARGAGYNFDGAYRFFDFWGQG |
| | | region | TLVTVSS |
| SEQ ID | BKM0195 | BKM0195 | QVQLVQSGAEVKKPGSSVKVSCKASGYTFTGYVMHWVRQ |
| NO: 517 | VH | N30S heavy | APGQGLEWMGFIIPIFGTANYAQKFQGRVTITSDKSTTT |
| | | chain variable | AYMELSSLRSEDTAVYYCARGAGYNFDGAYRFFDFWGQG |
| | | region | TLVTVSS |
| SEQ ID | BKM096 | BKM0196 | QVQLVQSGAEVKKPGSSVKVSCKASGYTFTGYVMHWVRQ |
| NO: 517 | VH | N28G N30S | APGQGLEWMGFIIPIFGTANYAQKFQGRVTITSDKSTTT |
| | | heavy chain | AYMELSSLRSEDTAVYYCARGAGYNFDGAYRFFDFWGQG |
| | | variable region | TLVTVSS |
| SEQ ID | h1JOVI.1- | JOVI.1 light | DVVMTQSPLSLPVTPGEPASISCRSSQRLVHSNGNTYLH |
| NO: 256 | L | chain variable | WYLQKPGQSPQLLIYRVSNRFPGVPDRFSGSGSGTDFTL |
| | | region | KISRVEAEDVGVYFCSQSTHVPYTFGGGTKVEIK |
| | | humanized | |
| | | variant 1 | |
| SEQ ID | h2JOVI.1- | JOVI.1 light | EVVMTQSPGTLSLSPGERATLSCRSSQRLVHSNGNTYLH |
| NO: 257 | L | chain variable | WYQQKPGQAPRLLIYRVSNRFPGIPDRFSGSGSGTDFTL |
| | | region | TISRLEPEDFAVYFCSQSTHVPYTFGGGTKVEIK |
| | | humanized | |
| | | variant 2 | |
| SEQ ID | h3JOVI.1- | JOVI.1 light | DVVMTQSPLSLPVTLGQPASISCRSSQRLVHSNGNTYLH |
| NO: 258 | L | chain variable | WYQQRPGQSPRLLIYRVSNRFPGVPDRFSGSGSGTDFTL |
| | | region | KISRVEAEDVGVYFCSQSTHVPYTFGGGTKVEIK |
| | | humanized | |
| | | variant 3 | |
| SEQ ID | h4JOVI.1- | JOVI.1 light | DVVMTQTPLSLPVTPGEPASISCRSSQRLVHSNGNTYLH |
| NO: 259 | L | chain variable | WYLQKPGQSPQLLIYRVSNRFPGVPDRFSGSGSGTDFTL |
| | | region | KISRVEAEDVGVYFCSQSTHVPYTFGGGTKVEIK |
| | | humanized | |
| | | variant 4 | |
| SEQ ID | h5JOVI.1- | JOVI.1 light | DVVMTQTPLSLSVTPGQPASISCRSSQRLVHSNGNTYLH |
| NO: 260 | L | chain variable | WYLQKPGQSPQLLIYRVSNRFPGVPDRFSGSGSGTDFTL |
| | | region | KISRVEAEDVGVYFCSQSTHVPYTFGGGTKVEIK |
| | | humanized | |
| | | variant 5 | |
| SEQ ID | L1 | JOVI.1 light | DVVMTQSPLSLPVTLGQPASISCRSSQSLVYSDGNTYHW |
| NO: | germlined- | chain variable | YQQRPGQSPRLLIYRVSNRFPGVPDRFSGSGSGTDFTLK |
| 7357 | VL | region | ISRVEAEDVGVYFCSQSTHVPYTFGGGTKVEIK |
| | | humanized L1 | |
| SEQ ID | L2 | JOVI.1 light | DVVMTQSPLSLPVTLGQPASISCRSSQRLVHSNGNTYLH |
| NO: | germlined- | chain variable | WYQQRPGQSPRLLIYKVSNRDSGVPDRFSGSGSGTDFTL |
| 7358 | VL | region | KISRVEAEDVGVYFCSQSTHVPYTFGGGTKVEIK |
| | | humanized L2 | |
| SEQ ID | L3 | JOVI.1 light | DVVMTQSPLSLPVTLGQPASISCRSSQRLVHSNGNTYLH |
| NO: | germlined- | chain variable | WYQQRPGQSPRLLIYRVSNRFPGVPDRFSGSGSGTDFTL |
| 7359 | VL | region | KISRVEAEDVGVYFCMQSTHWPYTFGGGTKVEIK |
| | | humanized L3 | |
| SEQ ID | L1/L2/L3 | JOVI.1 light | DVVMTQSPLSLPVTLGQPASISCRSSQSLVYSDGNTYHW |
| NO: | germlined- | chain variable | YQQRPGQSPRLLIYKVSNRDSGVPDRFSGSGSGTDFTLK |
| 7360 | VL | region | ISRVEAEDVGVYFCMQSTHWPYTFGGGTKVEIK |
| | | humanized | |
| | | L1/L2/L3 | |
| SEQ ID | BKM0191 | BKM0191 | DVVMTQSPLSLPVTLGQPASISCRSSQRLVHSNANTYLH |
| NO: 374 | VL | G29A light | WYQQRPGQSPRLLIYRVSNRFPGVPDRFSGSGSGTDFTL |
| | | chain variable | KISRVEAEDVGVYFCSQSTHVPYTFGGGTKVEIK |
| | | region | |
| SEQ ID | BKM0192 | BKM0192 | DVVMTQSPLSLPVTLGQPASISCRSSQRLVHSNGNAYLH |
| NO: 375 | VL | T31A light | WYQQRPGQSPRLLIYRVSNRFPGVPDRFSGSGSGTDFTL |
| | | chain variable | KISRVEAEDVGVYFCSQSTHVPYTFGGGTKVEIK |
| | | region | |
| SEQ ID | BKM0193 | BKM0193 | DVVMTQSPLSLPVTLGQPASISCRSSQRLVHSNANAYLH |
| NO: 376 | VL | G29a T31A | WYQQRPGQSPRLLIYRVSNRFPGVPDRFSGSGSGTDFTL |
| | | light chain | KISRVEAEDVGVYFCSQSTHVPYTFGGGTKVEIK |
| | | variable region | |
| SEQ ID | BKM0194 | BKM0194 | DVVMTQSPLSLPVTLGQPASISCRSSQRLVHSGGNTYLH |
| NO: 377 | VL | N28G light | WYQQRPGQSPRLLIYRVSNRFPGVPDRFSGSGSGTDFTL |
| | | chain variable | KISRVEAEDVGVYFCSQSTHVPYTFGGGTKVEIK |
| | | region | |
| SEQ ID | BKM0195 | BKM0195 | DVVMTQSPLSLPVTLGQPASISCRSSQRLVHSNGSTYLH |
| NO: 378 | VL | N30S light | WYQQRPGQSPRLLIYRVSNRFPGVPDRFSGSGSGTDFTL |
| | | chain variable | KISRVEAEDVGVYFCSQSTHVPYTFGGGTKVEIK |
| | | region | |
| SEQ ID | BKM0196 | BKM0196 | DVVMTQSPLSLPVTLGQPASISCRSSQRLVHSGGSTYLH |
| NO: 379 | VL | N28G N30S | WYQQRPGQSPRLLIYRVSNRFPGVPDRFSGSGSGTDFTL |
| | | light chain | KISRVEAEDVGVYFCSQSTHVPYTFGGGTKVEIK |
| | | variable region | |

TABLE 8
Exemplary TRBC1-targeting antigen binding domains/antibody molecules

| SEQ ID | | | |
|---|---|---|---|
| NO | Ab ID | Description | Sequence |
| SEQ ID | Ch(anti- | Anti-TRBC1 | EVRLQQSGPDLIKPGASVKMSCKASGYTFTGYVMHWVKQR |
| NO: | TRBC1) | heavy chain | PGQGLEWIGFINPYNDDIQSNERFRGKATLTSDKSSTTAYME |
| 6154 | HC | | LSSLTSEDSAVYYCARGAGYNFDGAYRFFDFWGQGTTLTV |
| | N297A | | SSASTKGPSVFPLAPSSKSTSGGTAALGCLVKDYFPEPVTVS |
| | | | WNSGALTSGVHTFPAVLQSSGLYSLSSVVTVPSSSLGTQTYI |
| | | | CNVNHKPSNTKVDKRVEPKSCDKTHTCPPCPAPELLGGPSV |
| | | | FLFPPKPKDTLMISRTPEVTCVVVDVSHEDPEVKFNWYVDG |
| | | | VEVHNAKTKPREEQYASTYRVVSVLTVLHQDWLNGKEYK |
| | | | CKVSNKALPAPIEKTISKAKGQPREPQVYTLPPCREEMTKNQ |
| | | | VSLWCLVKGFYPSDIAVEWESNGQPENNYKTTPPVLDSDGS |
| | | | FFLYSKLTVDKSRWQQGNVFSCSVMHEALHNHYTQKSLSL |
| | | | SPGK |
| SEQ ID | Ch(anti- | Anti-TRBC1 | EVRLQQSGPDLIKPGASVKMSCKASGYTFTGYVMHWVKQR |
| NO: | TRBC1) | heavy chain | PGQGLEWIGFINPYNDDIQSNERFRGKATLTSDKSSTTAYME |
| 4671 | HC | | LSSLTSEDSAVYYCARGAGYNFDGAYRFFDFWGQGTTLTV |
| | N297A | | SSASTKGPSVFPLAPSSKSTSGGTAALGCLVKDYFPEPVTVS |
| | | | WNSGALTSGVHTFPAVLQSSGLYSLSSVVTVPSSSLGTQTYI |
| | | | CNVNHKPSNTKVDKRVEPKSCDKTHTCPPCPAPELLGGPSV |
| | | | FLFPPKPKDTLMISRTPEVTCVVVDVSHEDPEVKFNWYVDG |
| | | | VEVHNAKTKPREEQYASTYRVVSVLTVLHQDWLNGKEYK |
| | | | CKVSNKALPAPIEKTISKAKGQPREPQVYTLPPCREEMTKNQ |
| | | | VSLWCLVKGFYPSDIAVEWESNGQPENNYKTTPPVLDSDGS |
| | | | FFLYSKLTVDKSRWQQGNVFSCSVMHEALHNHYTQKSLSL |
| | | | SP |
| SEQ ID | Ch(anti- | Anti-TRBC1 | EVRLQQSGPDLIKPGASVKMSCKASGYTFTGYVMHWVKQR |
| NO: | TRBC1) | heavy chain | PGQGLEWIGFINPYNDDIQSNERFRGKATLTSDKSSTTAYME |
| 6155 | HC | | LSSLTSEDSAVYYCARGAGYNFDGAYRFFDFWGQGTTLTV |
| | | | SSASTKGPSVFPLAPSSKSTSGGTAALGCLVKDYFPEPVTVS |
| | | | WNSGALTSGVHTFPAVLQSSGLYSLSSVVTVPSSSLGTQTYI |
| | | | CNVNHKPSNTKVDKRVEPKSCDKTHTCPPCPAPELLGGPSV |
| | | | FLFPPKPKDTLMISRTPEVTCVVVDVSHEDPEVKFNWYVDG |
| | | | VEVHNAKTKPREEQYNSTYRVVSVLTVLHQDWLNGKEYK |
| | | | CKVSNKALPAPIEKTISKAKGQPREPQVYTLPPCREEMTKNQ |
| | | | VSLWCLVKGFYPSDIAVEWESNGQPENNYKTTPPVLDSDGS |
| | | | FFLYSKLTVDKSRWQQGNVFSCSVMHEALHNHYTQKSLSL |
| | | | SPGK |
| SEQ ID | Ch(anti- | Anti-TRBC1 | EVRLQQSGPDLIKPGASVKMSCKASGYTFTGYVMHWVKQR |
| NO: | TRBC1) | heavy chain | PGQGLEWIGFINPYNDDIQSNERFRGKATLTSDKSSTTAYME |
| 4672 | HC | | LSSLTSEDSAVYYCARGAGYNFDGAYRFFDFWGQGTTLTV |
| | | | SSASTKGPSVFPLAPSSKSTSGGTAALGCLVKDYFPEPVTVS |
| | | | WNSGALTSGVHTFPAVLQSSGLYSLSSVVTVPSSSLGTQTYI |
| | | | CNVNHKPSNTKVDKRVEPKSCDKTHTCPPCPAPELLGGPSV |
| | | | FLFPPKPKDTLMISRTPEVTCVVVDVSHEDPEVKFNWYVDG |
| | | | VEVHNAKTKPREEQYNSTYRVVSVLTVLHQDWLNGKEYK |
| | | | CKVSNKALPAPIEKTISKAKGQPREPQVYTLPPCREEMTKNQ |
| | | | VSLWCLVKGFYPSDIAVEWESNGQPENNYKTTPPVLDSDGS |
| | | | FFLYSKLTVDKSRWQQGNVFSCSVMHEALHNHYTQKSLSL |
| | | | SP |
| SEQ ID | Ch(anti- | Anti-TRBC1 | DVVMTQSPLSLPVSLGDQASISCRSSQRLVHSNGNTYLHWY |
| NO: | TRBC1) | light chain, | LQKPGQSPKLLIYRVSNRFPGVPDRFSGSGSGTDFTLKISRVE |
| 6156 | LC | e.g., a LC Fab | AEDLGIYFCSQSTHVPYTFGGGTKLEIKRTVAAPSVFIFPPSD |
| | | | EQLKSGTASVVCLLNNFYPREAKVQWKVDNALQSGNSQES |
| | | | VTEQDSKDSTYSLSSTLTLSKADYEKHKVYACEVTHQGLSS |
| | | | PVTKSFNRGEC |
| SEQ ID | Ch(anti- | Anti-TRBC1 | EVRLQQSGPDLIKPGASVKMSCKASGYTFTGYVMHWVKQR |
| NO: | TRBC1) | heavy chain, | PGQGLEWIGFINPYNDDIQSNERFRGKATLTSDKSSTTAYME |
| 6191 | HC | e.g., a HC Fab | LSSLTSEDSAVYYCARGAGYNFDGAYRFFDFWGQGTTLTV |
| | | | SSASTKGPSVFPLAPSSKSTSGGTAALGCLVKDYFPEPVTVS |
| | | | WNSGALTSGVHTFPAVLQSSGLYSLSSVVTVPSSSLGTQTYI |
| | | | CNVNHKPSNTKVDKRVEPKSC |
| SEQ ID | a_hTRBC1_ | Anti-TRBC1 | METDTLLLWVLLLWVPGSTGQVQLVQSGAEVKKPGSSVKV |
| NO: | Jovi1_ | heavy chain | SCKASGYTFTGYVMHWVRQAPGQGLEWMGFINPYNDDIQS |
| 6167 | Hum5_ | | NERFRGRVTITSDKSTTTAYMELSSLRSEDTAVYYCARGAG |
| | VH- | | YNFDGAYRFFDFWGQGTLVTVSSASTKGPSVFPLAPSSKSTS |
| | hCHIg_ | | GGTAALGCLVKDYFPEPVTVSWNSGALTSGVHTFPAVLQSS |
| | Hole_ | | GLYSLSSVVTVPSSSLGTQTYICNVNHKPSNTKVDKRVEPKS |
| | Cys- | | CDKTHTCPPCPAPELLGGPSVFLFPPKPKDTLMISRTPEVTCV |
| | Blank | | VVDVSHEDPEVKFNWYVDGVEVHNAKTKPREEQYNSTYR |
| | | | VVSVLTVLHQDWLNGKEYKCKVSNKALPAPIEKTISKAKG |
| | | | QPREPQVCTLPPSREEMTKNQVSLSCAVKGFYPSDIAVEWE |
| | | | SNGQPENNYKTTPPVLDSDGSFFLVSKLTVDKSRWQQGNVF |
| | | | SCSVMHEALHNHYTQKSLSLSPGK |
| SEQ ID | a_hTRBC1_ | Anti-TRBC1 | METDTLLLWVLLLWVPGSTGQVQLVQSGAEVKKPGSSVKV |
| NO: | Jovi1_ | heavy chain | SCKASGYTFTGYVMHWVRQAPGQGLEWMGFINPYNDDIQS |
| 4673 | Hum5_ | | NERFRGRVTITSDKSTTTAYMELSSLRSEDTAVYYCARGAG |
| | VH- | | YNFDGAYRFFDFWGQGTLVTVSSASTKGPSVFPLAPSSKSTS |
| | hCHIg_ | | GGTAALGCLVKDYFPEPVTVSWNSGALTSGVHTFPAVLQSS |
| | Hole_ | | GLYSLSSVVTVPSSSLGTQTYICNVNHKPSNTKVDKRVEPKS |
| | Cys- | | CDKTHTCPPCPAPELLGGPSVFLFPPKPKDTLMISRTPEVTCV |
| | Blank | | VVDVSHEDPEVKFNWYVDGVEVHNAKTKPREEQYNSTYR |
| | | | VVSVLTVLHQDWLNGKEYKCKVSNKALPAPIEKTISKAKG |
| | | | QPREPQVCTLPPSREEMTKNQVSLSCAVKGFYPSDIAVEWE |
| | | | SNGQPENNYKTTPPVLDSDGSFFLVSKLTVDKSRWQQGNVF |
| | | | SCSVMHEALHNHYTQKSLSLSP |
| SEQ ID | a_hTRBC1_ | Anti-TRBC1 | METDTLLLWVLLLWVPGSTGQVQLVQSGAEVKKPGSSVKV |
| NO: | Jovi1_ | heavy chain | SCKASGYTFTGYVMHWVRQAPGQGLEWMGFINPYNDDIQS |
| 6168 | Hum5_ | | NERFRGRVTITSDKSTTTAYMELSSLRSEDTAVYYCARGAG |
| | VH- | | YNFDGAYRFFDFWGQGTLVTVSSASTKGPSVFPLAPSSKSTS |
| | hCHIg- | | GGTAALGCLVKDYFPEPVTVSWNSGALTSGVHTFPAVLQSS |
| | Blank | | GLYSLSSVVTVPSSSLGTQTYICNVNHKPSNTKVDKRVEPKS |
| | | | CDKTHTCPPCPAPELLGGPSVFLFPPKPKDTLMISRTPEVTCV |
| | | | VVDVSHEDPEVKFNWYVDGVEVHNAKTKPREEQYNSTYR |
| | | | VVSVLTVLHQDWLNGKEYKCKVSNKALPAPIEKTISKAKG |
| | | | QPREPQVYTLPPSREEMTKNQVSLTCLVKGFYPSDIAVEWE |
| | | | SNGQPENNYKTTPPVLDSDGSFFLYSKLTVDKSRWQQGNVF |
| | | | SCSVMHEALHNHYTQKSLSLSPGK |
| SEQ ID | a_hTRBC1_ | Anti-TRBC1 | METDTLLLWVLLLWVPGSTGQVQLVQSGAEVKKPGSSVKV |
| NO: | Jovi1_ | heavy chain | SCKASGYTFTGYVMHWVRQAPGQGLEWMGFINPYNDDIQS |
| 4674 | Hum5_ | | NERFRGRVTITSDKSTTTAYMELSSLRSEDTAVYYCARGAG |
| | VH- | | YNFDGAYRFFDFWGQGTLVTVSSASTKGPSVFPLAPSSKSTS |
| | hCHIg- | | GGTAALGCLVKDYFPEPVTVSWNSGALTSGVHTFPAVLQSS |
| | Blank | | GLYSLSSVVTVPSSSLGTQTYICNVNHKPSNTKVDKRVEPKS |
| | | | CDKTHTCPPCPAPELLGGPSVFLFPPKPKDTLMISRTPEVTCV |
| | | | VVDVSHEDPEVKFNWYVDGVEVHNAKTKPREEQYNSTYR |
| | | | VVSVLTVLHQDWLNGKEYKCKVSNKALPAPIEKTISKAKG |
| | | | QPREPQVYTLPPSREEMTKNQVSLTCLVKGFYPSDIAVEWE |
| | | | SNGQPENNYKTTPPVLDSDGSFFLYSKLTVDKSRWQQGNVF |
| | | | SCSVMHEALHNHYTQKSLSLSP |
| SEQ ID | a_hTRBC1_ | Anti-TRBC1 | METDTLLLWVLLLWVPGSTGDVVMTQSPLSLPVTLGQPASI |
| NO: | Jovi1_ | light chain | SCRSSQRLVHSNGNTYLHWYQQRPGQSPRLLIYRVSNRFPG |
| 6169 | Hum3_ | | VPDRFSGSGSGTDFTLKISRVEAEDVGVYFCSQSTHVPYTFG |
| | VL-hCLIg_ | | GGTKVEIKRTVAAPSVFIFPPSDEQLKSGTASVVCLLNNFYP |
| | vk-Blank | | REAKVQWKVDNALQSGNSQESVTEQDSKDSTYSLSSTLTLS |
| | | | KADYEKHKVYACEVTHQGLSSPVTKSFNRGEC |

TABLE 9A
Exemplary heavy chain CDRs and FWRs of
TRBC2-targeting antigen binding domains
(According to Kabat numbering scheme)

| Ab ID | VHFWR1 | VHCDR1 | VHFWR2 | VHCDR2 | VHFWR3 | VHCDR3 | VHFWR4 |
|---|---|---|---|---|---|---|---|
| JVD3- | QVQLVQS | GFVMH | WVRQAP | FINPYND | RVTMTS | GNGMWF | WGQGT |
| VH | GAEVKKP | (SEQ ID | GQGLEW | DIQSNER | DKSTTTA | DGAYRFF | LVTVSS |
| | GASVKVS | NO: | MG (SEQ | FRG (SEQ | YMELSSL | DF (SEQ | (SEQ ID |
| | CKASGYT | 7394) | ID NO: | ID NO: | RSEDTAV | ID NO: | NO: 210) |
| | FP (SEQ ID | | 208) | 201) | YYCAR | 307) | |
| | NO: 7393) | | | | (SEQ ID | | |
| | | | | | NO: 209) | | |
| JVD4- | QVQLVQS | GYVMH | WVRQAP | FINPYND | RVTMTS | GNGKNF | WGQGT |
| VH | GAEVKKP | (SEQ ID | GQGLEW | DIQSNER | DKSTTTA | DGAYRFF | LVTVSS |
| | GASVKVS | NO: | MG (SEQ | FRG (SEQ | YMELSSL | DF (SEQ | (SEQ ID |
| | CKASGYP | 8643) | ID NO: | ID NO: | RSEDTAV | ID NO: | NO: 210) |
| | FH (SEQ | | 208) | 201) | YYCAR | 8695) | |
| | ID NO: | | | | (SEQ ID | | |
| | 7397) | | | | NO: 209) | | |
| JVD5- | QVQLVQS | GYVMH | WVRQAP | FINPYND | RVTMTS | GNGKWF | WGQGT |
| VH | GAEVKKP | (SEQ ID | GQGLEW | DIQSNER | DKSTTTA | DGAYRFF | LVTVSS |
| | GASVKVS | NO: | MG (SEQ | FRG (SEQ | YMELSSL | DF (SEQ | (SEQ ID |
| | CKASGYT | 8643) | ID NO: | ID NO: | RSEDTAV | ID NO: | NO: 210) |
| | YP (SEQ | | 208) | 201) | YYCAR | 7400) | |
| | ID NO: | | | | (SEQ ID | | |
| | 7399) | | | | NO: 209) | | |
| JVD6- | QVQLVQS | GYHMH | WVRQAP | FINPYND | RVTMTS | GNGKWG | WGQGT |
| VH | GAEVKKP | (SEQ ID | GQGLEW | DIQSNER | DKSTTTA | DGAYRFF | LVTVSS |
| | GASVKVS | NO: | MG (SEQ | FRG (SEQ | YMELSSL | DF (SEQ | (SEQ ID |
| | CKASNQN | 7401) | ID NO: | ID NO: | RSEDTAV | ID NO: | NO: 210) |
| | FH (SEQ | | 208) | 201) | YYCAR | 7403) | |
| | ID NO: | | | | (SEQ ID | | |
| | 8696) | | | | NO: 209) | | |
| JVD7- | QVQLVQS | GFYMH | WVRQAP | FINPYND | RVTMTS | GNGKWG | WGQGT |
| VH | GAEVKKP | (SEQ ID | GQGLEW | DIQSNER | DKSTTTA | DGAYRFF | LVTVSS |
| | GASVKVS | NO: | MG (SEQ | FRG (SEQ | YMELSSL | DF (SEQ | (SEQ ID |
| | CKASSQN | 7405) | ID NO: | ID NO: | RSEDTAV | ID NO: | NO: 210) |
| | FH (SEQ | | 208) | 201) | YYCAR | 7403) | |
| | ID NO: | | | | (SEQ ID | | |
| | 7404) | | | | NO: 209) | | |
| JVD8- | QVQLVQS | GYKMH | WVRQAP | FINPYND | RVTMTS | GNGKWG | WGQGT |
| VH | GAEVKKP | (SEQ ID | GQGLEW | DIQSNER | DKSTTTA | DGAYRFF | LVTVSS |
| | GASVKVS | NO: | MG (SEQ | FRG (SEQ | YMELSSL | DF (SEQ | (SEQ ID |
| | CKASYQD | 7407) | ID NO: | ID NO: | RSEDTAV | ID NO: | NO: 210) |
| | FH (SEQ | | 208) | 201) | YYCAR | 7403) | |
| | ID NO: | | | | (SEQ ID | | |
| | 7406) | | | | NO: 209) | | |
| JVD9- | QVQLVQS | GFYMH | WVRQAP | FINPYND | RVTMTS | GNGKWG | WGQGT |
| VH | GAEVKKP | (SEQ ID | GQGLEW | DIQSNER | DKSTTTA | DGAYRFF | LVTVSS |
| | GASVKVS | NO: | MG (SEQ | FRG (SEQ | YMELSSL | DF (SEQ | (SEQ ID |
| | CKASGYN | 7405) | ID NO: | ID NO: | RSEDTAV | ID NO: | NO: 210) |
| | FH (SEQ | | 208) | 201) | YYCAR | 7403) | |
| | ID NO: | | | | (SEQ ID | | |
| | 7408) | | | | NO: 209) | | |
| BKM0097 | QVQLVQS | GYPMH | WVRQAP | FINPYND | RVTMTS | GNGKWG | WGQGT |
| anti- | GAEVKKP | (SEQ ID | GQGLEW | DIQSNER | DKSTTTA | DGAYRFF | LVTVSS |
| TRBC2 | GASVKVS | NO: | MG (SEQ | FRG (SEQ | YMELSSL | DF (SEQ | (SEQ ID |
| VH; | CKASTSGF | 7422) | ID NO: | ID NO: | RSEDTAV | ID NO: | NO: 210) |
| BJM1184 | H (SEQ ID | | 208) | 201) | YYCAR | 7403) | |
| VH | NO: 7421) | | | | (SEQ ID | | |
| | | | | | NO: 209) | | |
| BKM0098 | QVQLVQS | GYHMH | WVRQAP | FINPYND | RVTMTS | GNGKWG | WGQGT |
| anti- | GAEVKKP | (SEQ ID | GQGLEW | DIQSNER | DKSTTTA | DGAYRFF | LVTVSS |
| TRBC2 | GASVKVS | NO: | MG (SEQ | FRG (SEQ | YMELSSL | DF (SEQ | (SEQ ID |
| VH; | CKASPRGF | 7401) | ID NO: | ID NO: | RSEDTAV | ID NO: | NO: 210) |
| BJM1185 | H (SEQ ID | | 208) | 201) | YYCAR | 7403) | |
| VH | NO: 7424) | | | | (SEQ ID | | |
| | | | | | NO: 209) | | |
| BJM1186 | QVQLVQS | GYAMH | WVRQAP | FINPYND | RVTMTS | GNGKWG | WGQGT |
| VH | GAEVKKP | (SEQ ID | GQGLEW | DIQSNER | DKSTTTA | DGAYRFF | LVTVSS |
| | GASVKVS | NO: | MG (SEQ | FRG (SEQ | YMELSSL | DF (SEQ | (SEQ ID |
| | CKASFQD | 7427) | ID NO: | ID NO: | RSEDTAV | ID NO: | NO: 210) |
| | FH (SEQ | | 208) | 201) | YYCAR | 7403) | |
| | ID NO: | | | | (SEQ ID | | |
| | 7426) | | | | NO: 209) | | |
| BJM1187 | QVQLVQS | GFAMH | WVRQAP | FINPYND | RVTMTS | GNGKWG | WGQGT |
| VH | GAEVKKP | (SEQ ID | GQGLEW | DIQSNER | DKSTTTA | DGAYRFF | LVTVSS |
| | GASVKVS | NO: | MG (SEQ | FRG (SEQ | YMELSSL | DF (SEQ | (SEQ ID |
| | CKASSKD | 7430) | ID NO: | ID NO: | RSEDTAV | ID NO: | NO: 210) |
| | FH (SEQ | | 208) | 201) | YYCAR | 7403) | |
| | ID NO: | | | | (SEQ ID | | |
| | 7429) | | | | NO: 209) | | |
| BC2_YR3_ | QVQLVQS | GYHMH | WVRQAP | FINPYND | RVTMTS | GNGKWG | WGQGT |
| B11-scFv | GAEVKKP | (SEQ ID | GQGLEW | DIQSNER | DKSTTTA | DGAYRFF | LVTVSS |
| VH | GASVKVS | NO: | MG (SEQ | FRG (SEQ | YMELSSL | DF (SEQ | (SEQ ID |
| | CKASPKG | 7401) | ID NO: | ID NO: | RSEDTAV | ID NO: | NO: 210) |
| | FH (SEQ | | 208) | 201) | YYCAR | 7403) | |
| | ID NO: | | | | (SEQ ID | | |
| | 7432) | | | | NO: 209) | | |
| Consensus | | GX1X2MH, | | FINPYND | | GNGX1X2 | |
| VHCDR | | wherein | | DIQSNER | | X3DGAYR | |
| | | X1 is Y | | FRG (SEQ | | FFDF | |
| | | or F, and | | ID NO: | | wherein | |
| | | X2 is P, | | 201) | | X1 is K | |
| | | H, V, Y, | | | | or M, X2 | |
| | | K, or A | | | | is W or N, | |
| | | | | | | and X3 is | |
| | | | | | | G or F | |
| | | | | | | (SEQ ID | |
| | | | | | | NO: 7442) | |

TABLE 9B
Exemplary heavy chain CDRs and FWRs of TRBC2-targeting antigen binding domains
of Table 9A (According to ABM numbering scheme)

| Ab ID | VHFWR1 | VHCDR1 | VHFWR2 | VHCDR2 | VHFWR3 | VHCDR3 | VHFWR4 |
|---|---|---|---|---|---|---|---|
| JVD3- | QVQLVQSG | GYTFP | WVRQAP | FINPYND | RVTMTSD | GNGMWF | WGQGT |
| VH | AEVKKPGA | GFVMH | GQGLEW | DIQSNER | KSTTTAY | DGAYRFF | LVTVSS |
| | SVKVSCKA | (SEQ ID | MG (SEQ | FRG (SEQ | MELSSLRS | DF (SEQ | (SEQ ID |
| | S (SEQ ID | NO: | ID NO: | ID NO: | EDTAVYY | ID NO: | NO: 210) |
| | NO: 8528) | 8529) | 208) | 201) | CAR (SEQ | 307) | |
| | | | | | ID NO: | | |
| | | | | | 209) | | |
| JVD4- | QVQLVQSG | GYPFH | WVRQAP | FINPYND | RVTMTSD | GNGKNF | WGQGT |
| VH | AEVKKPGA | GYVMH | GQGLEW | DIQSNER | KSTTTAY | DGAYRFF | LVTVSS |
| | SVKVSCKA | (SEQ ID | MG (SEQ | FRG (SEQ | MELSSLRS | DF (SEQ | (SEQ ID |
| | S (SEQ ID | NO: | ID NO: | ID NO: | EDTAVYY | ID NO: | NO: 210) |
| | NO: 8530) | 8531) | 208) | 201) | CAR (SEQ | 8695) | |
| | | | | | ID NO: | | |
| | | | | | 209) | | |
| JVD5- | QVQLVQSG | GYTYP | WVRQAP | FINPYND | RVTMTSD | GNGKWF | WGQGT |
| VH | AEVKKPGA | GYVMH | GQGLEW | DIQSNER | KSTTTAY | DGAYRFF | LVTVSS |
| | SVKVSCKA | (SEQ ID | MG (SEQ | FRG (SEQ | MELSSLRS | DF (SEQ | (SEQ ID |
| | S (SEQ ID | NO: | ID NO: | ID NO: | EDTAVYY | ID NO: | NO: 210) |
| | NO: 8532) | 8533) | 208) | 201) | CAR (SEQ | 7400) | |
| | | | | | ID NO: | | |
| | | | | | 209) | | |
| JVD6- | QVQLVQSG | NQNFH | WVRQAP | FINPYND | RVTMTSD | GNGKWG | WGQGT |
| VH | AEVKKPGA | GYHMH | GQGLEW | DIQSNER | KSTTTAY | DGAYRFF | LVTVSS |
| | SVKVSCKA | (SEQ ID | MG (SEQ | FRG (SEQ | MELSSLRS | DF (SEQ | (SEQ ID |
| | S (SEQ ID | NO: | ID NO: | ID NO: | EDTAVYY | ID NO: | NO: 210) |
| | NO: 8534) | 8535) | 208) | 201) | CAR (SEQ | 7403) | |
| | | | | | ID NO: | | |
| | | | | | 209) | | |
| JVD7- | QVQLVQSG | SQNFH | WVRQAP | FINPYND | RVTMTSD | GNGK WG | WGQGT |
| VH | AEVKKPGA | GFYMH | GQGLEW | DIQSNER | KSTTTAY | DGAYRFF | LVTVSS |
| | SVKVSCKA | (SEQ ID | MG (SEQ | FRG (SEQ | MELSSLRS | DF (SEQ | (SEQ ID |
| | S (SEQ ID | NO: | ID NO: | ID NO: | EDTAVYY | ID NO: | NO: 210) |
| | NO: 8536) | 8537) | 208) | 201) | CAR (SEQ | 7403) | |
| | | | | | ID NO: | | |
| | | | | | 209) | | |
| JVD8- | QVQLVQSG | YQDFH | WVRQAP | FINPYND | RVTMTSD | GNGKWG | WGQGT |
| VH | AEVKKPGA | GYKMH | GQGLEW | DIQSNER | KSTTTAY | DGAYRFF | LVTVSS |
| | SVKVSCKA | (SEQ ID | MG (SEQ | FRG (SEQ | MELSSLRS | DF (SEQ | (SEQ ID |
| | S (SEQ ID | NO: | ID NO: | ID NO: | EDTAVYY | ID NO: | NO: 210) |
| | NO: 8538) | 8539) | 208) | 201) | CAR (SEQ | 7403) | |
| | | | | | ID NO: | | |
| | | | | | 209) | | |
| JVD9- | QVQLVQSG | GYNFH | WVRQAP | FINPYND | RVTMTSD | GNGKWG | WGQGT |
| VH | AEVKKPGA | GFYMH | GQGLEW | DIQSNER | KSTTTAY | DGAYRFF | LVTVSS |
| | SVKVSCKA | (SEQ ID | MG (SEQ | FRG (SEQ | MELSSLRS | DF (SEQ | (SEQ ID |
| | S (SEQ ID | NO: | ID NO: | ID NO: | EDTAVYY | ID NO: | NO: 210) |
| | NO: 8540) | 8541) | 208) | 201) | CAR (SEQ | 7403) | |
| | | | | | ID NO: | | |
| | | | | | 209) | | |
| BKM0097 | QVQLVQSG | TSGFH | WVRQAP | FINPYND | RVTMTSD | GNGKWG | WGQGT |
| anti- | AEVKKPGA | GYPMH | GQGLEW | DIQSNER | KSTTTAY | DGAYRFF | LVTVSS |
| TRBC2 | SVKVSCKA | (SEQ ID | MG (SEQ | FRG (SEQ | MELSSLRS | DF (SEQ | (SEQ ID |
| VH; | S (SEQ ID | NO: | ID NO: | ID NO: | EDTAVYY | ID NO: | NO: 210) |
| BJM1184 | NO: 8542) | 8543) | 208) | 201) | CAR (SEQ | 7403) | |
| VH | | | | | ID NO: | | |
| | | | | | 209) | | |
| BKM0098 | QVQLVQSG | PRGFH | WVRQAP | FINPYND | RVTMTSD | GNGKWG | WGQGT |
| anti- | AEVKKPGA | GYHMH | GQGLEW | DIQSNER | KSTTTAY | DGAYRFF | LVTVSS |
| TRBC2 | SVKVSCKA | (SEQ ID | MG (SEQ | FRG (SEQ | MELSSLRS | DF (SEQ | (SEQ ID |
| VH; | S (SEQ ID | NO: | ID NO: | ID NO: | EDTAVYY | ID NO: | NO: 210) |
| BJM1185 | NO: 8544) | 8545) | 208) | 201) | CAR (SEQ | 7403) | |
| VH | | | | | ID NO: | | |
| | | | | | 209) | | |
| BJM1186 | QVQLVQSG | GYAMH | WVRQAP | FINPYND | RVTMTSD | GNGKWG | WGQGT |
| VH | AEVKKPGA | (SEQ ID | GQGLEW | DIQSNER | KSTTTAY | DGAYRFF | LVTVSS |
| | SVKVSCKA | NO: | MG (SEQ | FRG (SEQ | MELSSLRS | DF (SEQ | (SEQ ID |
| | SFQDFH | 8547) | ID NO: | ID NO: | EDTAVYY | ID NO: | NO: 210) |
| | (SEQ ID | | 208) | 201) | CAR (SEQ | 7403) | |
| | NO: 8546) | | | | ID NO: | | |
| | | | | | 209) | | |
| BJM1187 | QVQLVQSG | SKDFH | WVRQAP | FINPYND | RVTMTSD | GNGKWG | WGQGT |
| VH | AEVKKPGA | GFAMH | GQGLEW | DIQSNER | KSTTTAY | DGAYRFF | LVTVSS |
| | SVKVSCKA | (SEQ ID | MG (SEQ | FRG (SEQ | MELSSLRS | DF (SEQ | (SEQ ID |
| | S (SEQ ID | NO: | ID NO: | ID NO: | EDTAVYY | ID NO: | NO: 210) |
| | NO: 8548) | 8549) | 208) | 201) | CAR (SEQ | 7403) | |
| | | | | | ID NO: | | |
| | | | | | 209) | | |
| BC2_ | QVQLVQSG | PKGFH | WVRQAP | FINPYND | RVTMTSD | GNGKWG | WGQGT |
| YR3_B11- | AEVKKPGA | GYHMH | GQGLEW | DIQSNER | KSTTTAY | DGAYRFF | LVTVSS |
| scFv | SVKVSCKA | (SEQ ID | MG (SEQ | FRG (SEQ | MELSSLRS | DF (SEQ | (SEQ ID |
| VH | S (SEQ ID | NO: | ID NO: | ID NO: | EDTAVYY | ID NO: | NO: 210) |
| | NO: 8550) | 8551) | 208) | 201) | CAR (SEQ | 7403) | |
| | | | | | ID NO: | | |
| | | | | | 209) | | |
| Consensus | | GX1X2 | | FINPYND | | GNGX1X2 | |
| VHCDR | | MH, | | DIQSNER | | X3DGAY | |
| | | wherein | | FRG (SEQ | | RFFDF, | |
| | | X1 is Y | | ID NO: | | wherein X1 is | |
| | | or F, and | | 201) | | K or M, X2 | |
| | | X2 is P, | | | | is W or N, | |
| | | H, V, Y, | | | | and X3 is | |
| | | K, or A | | | | G or F | |
| | | | | | | (SEQ ID | |
| | | | | | | NO: 7442) | |

TABLE 10
Exemplary light chain CDRs and FWRs of TRBC2-targeting antigen binding domains

| Ab ID | VLFWR1 | VLCDR1 | VLFWR2 | VLCDR2 | VLFWR3 | VLCDR3 | VLFWR4 |
|---|---|---|---|---|---|---|---|
| JVD2- | DVVMTQS | RSSQNL | WYLQKP | RVSNRFP | GVPDRFS | SQSTHVP | FGGGTK |
| VL | PLSLPVTP | VHSNGR | GQSPQLL | (SEQ ID | GSGSGTDF | YT (SEQ | VEIK |
| | GEPASISC | TYLH | IY (SEQ | NO: 224) | TLKISRVE | ID NO: | (SEQ ID |
| | (SEQ ID | (SEQ ID | ID NO: | | AEDVGVY | 225) | NO: |
| | NO: 230) | NO: | 231) | | FC (SEQ ID | | 8685) |
| | | 7409) | | | NO: 240) | | |
| JVD34- | DVVMTQS | RSSQNL | WYLQKP | RVSNRFP | GVPDRFS | SQSTHVP | FGGGTK |
| VL | PLSLPVTP | VHSNGR | GQSPQLL | (SEQ ID | GSGSGTDF | YT (SEQ | VEIK |
| | GEPASISC | TYLQ | IY (SEQ | NO: 224) | TLKISRVE | ID NO: | (SEQ ID |
| | (SEQ ID | SEQ ID | ID NO: | | AEDVGVY | 225) | NO: |
| | NO: 230) | NO: | 231) | | FC (SEQ ID | | 8685) |
| | | 7410) | | | NO: 240) | | |
| Consensus | | RSSQNL | | RVSNRFP | | SQSTHVP | |
| LHCDR | | VHSNGR | | (SEQ ID | | YT (SEQ | |
| | | TYLX, | | NO: 224) | | ID NO: | |
| | | wherein X | | | | 225) | |
| | | is Q or H | | | | | |
| | | (SEQ ID | | | | | |
| | | NO: | | | | | |
| | | 7443) | | | | | |

TABLE 11
Exemplary variable regions of TRBC2-targeting antigen binding domains

| SEQ ID NO | Description | Sequence |
|---|---|---|
| SEQ ID | JVD3-VH | QVQLVQSGAEVKKPGASVKVSCKASGYTFPGFVMHWVRQAPGQGLEW |
| NO: 7411 | | MGFINPYNDDIQSNERFRGRVTMTSDKSTTTAYMELSSLRSEDTAVYYC |
| | | ARGNGMWFDGAYRFFDFWGQGTLVTVSS |
| SEQ ID | JVD4-VH | QVQLVQSGAEVKKPGASVKVSCKASGYPFHGYVMHWVRQAPGQGLE |
| NO: 7412 | | WMGFINPYNDDIQSNERFRGRVTMTSDKSTTTAYMELSSLRSEDTAVYY |
| | | CARGNGKNFDGAYRFFDFWGQGTLVTVSS |
| SEQ ID | JVD5-VH | QVQLVQSGAEVKKPGASVKVSCKASGYTYPGYVMHWVRQAPGQGLE |
| NO: 7413 | | WMGFINPYNDDIQSNERFRGRVTMTSDKSTTTAYMELSSLRSEDTAVYY |
| | | CARGNGKWFDGAYRFFDFWGQGTLVTVSS |
| SEQ ID | JVD6-VH | QVQLVQSGAEVKKPGASVKVSCKASNQNFHGYHMHWVRQAPGQGLE |
| NO: 7414 | | WMGFINPYNDDIQSNERFRGRVTMTSDKSTTTAYMELSSLRSEDTAVYY |
| | | CARGNGKWGDGAYRFFDFWGQGTLVTVSS |
| SEQ ID | JVD7-VH | QVQLVQSGAEVKKPGASVKVSCKASSQNFHGFYMHWVRQAPGQGLE |
| NO: 7415 | | WMGFINPYNDDIQSNERFRGRVTMTSDKSTTTAYMELSSLRSEDTAVYY |
| | | CARGNGKWGDGAYRFFDFWGQGTLVTVSS |
| SEQ ID | JVD8-VH | QVQLVQSGAEVKKPGASVKVSCKASYQDFHGYKMHWVRQAPGQGLE |
| NO: 7416 | | WMGFINPYNDDIQSNERFRGRVTMTSDKSTTTAYMELSSLRSEDTAVYY |
| | | CARGNGKWGDGAYRFFDFWGQGTLVTVSS |
| SEQ ID | JVD9-VH | QVQLVQSGAEVKKPGASVKVSCKASGYNFHGFYMHWVRQAPGQGLE |
| NO: 7417 | | WMGFINPYNDDIQSNERFRGRVTMTSDKSTTTAYMELSSLRSEDTAVYY |
| | | CARGNGKWGDGAYRFFDFWGQGTLVTVSS |
| SEQ ID | BKM0097 | QVQLVQSGAEVKKPGASVKVSCKASTSGFHGYPMHWVRQAPGQGLEW |
| NO: 7420 | anti-TRBC2 | MGFINPYNDDIQSNERFRGRVTMTSDKSTTTAYMELSSLRSEDTAVYYC |
| | VH; | ARGNGKWGDGAYRFFDFWGQGTLVTVSS |
| | BJM1184 | |
| | VH | |
| SEQ ID | BKM0098 | QVQLVQSGAEVKKPGASVKVSCKASPRGFHGYHMHWVRQAPGQGLE |
| NO: 7423 | anti-TRBC2 | WMGFINPYNDDIQSNERFRGRVTMTSDKSTTTAYMELSSLRSEDTAVYY |
| | VH; | CARGNGKWGDGAYRFFDFWGQGTLVTVSS |
| | BJM1185 | |
| | VH | |
| SEQ ID | BJM1186 | QVQLVQSGAEVKKPGASVKVSCKASFQDFHGYAMHWVRQAPGQGLE |
| NO: 7425 | VH | WMGFINPYNDDIQSNERFRGRVTMTSDKSTTTAYMELSSLRSEDTAVYY |
| | | CARGNGKWGDGAYRFFDFWGQGTLVTVSS |
| SEQ ID | BJM1187 | QVQLVQSGAEVKKPGASVKVSCKASSKDFHGFAMHWVRQAPGQGLE |
| NO: 7428 | VH | WMGFINPYNDDIQSNERFRGRVTMTSDKSTTTAYMELSSLRSEDTAVYY |
| | | CARGNGKWGDGAYRFFDFWGQGTLVTVSS |
| SEQ ID | BC2 YR3_ | QVQLVQSGAEVKKPGASVKVSCKASPKGFHGYHMHWVRQAPGQGLE |
| NO: 7431 | B11-scFv VH | WMGFINPYNDDIQSNERFRGRVTMTSDKSTTTAYMELSSLRSEDTAVYY |
| | | CARGNGKWGDGAYRFFDFWGQGTLVTVSS |
| SEQ ID | JVD2-VL | DVVMTQSPLSLPVTPGEPASISCRSSQNLVHSNGRTYLHWYLQKPGQSP |
| NO: 7418 | | QLLIYRVSNRFPGVPDRFSGSGSGTDFTLKISRVEAEDVGVYFCSQSTHV |
| | | PYTFGGGTKVEIK |
| SEQ ID | JVD34-VL | DVVMTQSPLSLPVTPGEPASISCRSSQNLVHSNGRTYLQWYLQKPGQSP |
| NO: 7419 | | QLLIYRVSNRFPGVPDRFSGSGSGTDFTLKISRVEAEDVGVYFCSQSTHV |
| | | PYTFGGGTKVEIK |

TABLE 12
Exemplary TRBC2-targeting antigen binding domains/antibody molecules

| SEQ ID NO | Description | Sequence |
|---|---|---|
| SEQ ID | BC2_YR3- | QVQLVQSGAEVKKPGASVKVSCKASTSGFHGYPMHWVRQAPGQGLEW |
| NO: 7433 | A12-scFv | MGFINPYNDDIQSNERFRGRVTMTSDKSTTTAYMELSSLRSEDTAVYYC |
| | (BJM1184) | ARGNGKWGDGAYRFFDFWGQGTLVTVSSGGGGSGGGGSGGGGSGGG |
| | | GSDVVMTQSPLSLPVTPGEPASISCRSSQNLVHSNGRTYLQWYLQKPGQ |
| | | SPQLLIYRVSNRFPGVPDRFSGSGSGTDFTLKISRVEAEDVGVYFCSQSTH |
| | | VPYTFGGGTKVEIK |
| SEQ ID | BC2_YR3- | QVQLVQSGAEVKKPGASVKVSCKASPRGFHGYHMHWVRQAPGQGLE |
| NO: 7434 | A5-scFv | WMGFINPYNDDIQSNERFRGRVTMTSDKSTTTAYMELSSLRSEDTAVYY |
| | (BJM1185) | CARGNGKWGDGAYRFFDFWGQGTLVTVSSGGGGSGGGGSGGGGSGG |
| | | GGSDVVMTQSPLSLPVTPGEPASISCRSSQNLVHSNGRTYLQWYLQKPG |
| | | QSPQLLIYRVSNRFPGVPDRFSGSGSGTDFTLKISRVEAEDVGVYFCSQST |
| | | HVPYTFGGGTKVEIK |
| SEQ ID | BC2_YR3- | QVQLVQSGAEVKKPGASVKVSCKASFQDFHGYAMHWVRQAPGQGLE |
| NO: 7435 | B3-scFv | WMGFINPYNDDIQSNERFRGRVTMTSDKSTTTAYMELSSLRSEDTAVYY |
| | (BJM1186) | CARGNGKWGDGAYRFFDFWGQGTLVTVSSGGGGSGGGGSGGGGSGG |
| | | GGSDVVMTQSPLSLPVTPGEPASISCRSSQNLVHSNGRTYLQWYLQKPG |
| | | QSPQLLIYRVSNRFPGVPDRFSGSGSGTDFTLKISRVEAEDVGVYFCSQST |
| | | HVPYTFGGGTKVEIK |
| SEQ ID | BC2_YR3_ | QVQLVQSGAEVKKPGASVKVSCKASSKDFHGFAMHWVRQAPGQGLE |
| NO: 7436 | B4-scFv | WMGFINPYNDDIQSNERFRGRVTMTSDKSTTTAYMELSSLRSEDTAVYY |
| | (BJM1187) | CARGNGKWGDGAYRFFDFWGQGTLVTVSSGGGGSGGGGSGGGGSGG |
| | | GGSDVVMTQSPLSLPVTPGEPASISCRSSQNLVHSNGRTYLQWYLQKPG |
| | | QSPQLLIYRVSNRFPGVPDRFSGSGSGTDFTLKISRVEAEDVGVYFCSQST |
| | | HVPYTFGGGTKVEIK |
| SEQ ID | BC2_YR3_ | QVQLVQSGAEVKKPGASVKVSCKASPKGFHGYHMHWVRQAPGQGLE |
| NO: 7437 | B11-scFv | WMGFINPYNDDIQSNERFRGRVTMTSDKSTTTAYMELSSLRSEDTAVYY |
| | | CARGNGKWGDGAYRFFDFWGQGTLVTVSSGGGGSGGGGSGGGGSGG |
| | | GGSDVVMTQSPLSLPVTPGEPASISCRSSQNLVHSNGRTYLQWYLQKPG |
| | | QSPQLLIYRVSNRFPGVPDRFSGSGSGTDFTLKISRVEAEDVGVYFCSQST |
| | | HVPYTFGGGTKVEIK |

TABLE 13
TRBC2 binding domains

| | Description | Sequence |
|---|---|---|
| TRCBC2 binder | TRBC2 -binding, | QVQLVQSGAEVKKPGSSVKVSCKASPRGFYGYHMHWVRQA |
| BKM0238 VH-1 - | Heavy Chain | PGQGLEWMGFINPYTNDIQYNERFRGRVTITSDESTTTAYME |
| SEQ ID NO: 8011 | Variable Region | LSSLRSEDTAVYYCAMGNGKWGDGAYRFFDLWGQGTLVT |
| | | VSS (SEQ ID NO: 8011) |
| TRCBC2 binder | TRBC2 -binding, | DVVMTQSPLSLPVTLGQPASISCRSSENLVHSNGRTYLQWYQ |
| BKM0238 VL-1 | Light Chain | QRPGQSPRLLIYRVSNRFPGVPDRFSGSGSGTDFTLKISRVEA |
| SEQ ID NO: 8012 | Variable Region | EDVGVYFCSQSSLEPYTFGGGTKVEIK (SEQ ID NO: 8012) |
| TRCBC2 binder | TRBC2 -binding, | QVQLVQSGAEVKKPGSSVKVSCKASPRGFYGYHMHWVRQA |
| BKM240 VH-2 - | Heavy Chain | PGQGLEWMGFINPYNNHIQYNERFRGRVTITSDESTTTAYME |
| SEQ ID NO: 8013 | | LSSLRSEDTAVYYCALGNGKWGDGAYRFFDFWGQGTLVTV |
| | | SS (SEQ ID NO: 8013) |
| TRCBC2 binder | TRBC2 -binding, | DVVMTQSPLSLPVTLGQPASISCRSSKNLVHSNGRTYLQWY |
| BKM240 VL-2 - | Light Chain | QQRPGQSPRLLIYRVSNRFPGVPDRFSGSGSGTDFTLKISRVE |
| SEQ ID NO: 8014 | | AEDVGVYFCSQSTREPYTFGGGTKVEIK (SEQ ID NO: 8014) |
| TRCBC2 binder | TRBC2 -binding, | QVQLVQSGAEVKKPGSSVKVSCKASPRGFYGYHMHWVRQA |
| BKM0311 VH-3 - | Heavy Chain | PGQGLEWMGFINPYNNHIQYNERFRGRVTITSDESTTTAYME |
| SEQ ID NO: 8020 | | LSSLRSEDTAVYYCALGEGKWGDGAYRFFDFWGQGTLVTV |
| | | SS (SEQ ID NO: 8020) |
| TRCBC2 binder | TRBC2 -binding, | DVVMTQSPLSLPVTLGQPASISCRSSKNLVHSNGRTYLQWY |
| BKM0311 VL-3 - | Light Chain | QQRPGQSPRLLIYRVSNRFPGVPDRFSGSGSGTDFTLKISRVE |
| SEQ ID NO: 8014 | | AEDVGVYFCSQSTREPYTFGGGTKVEIK (SEQ ID NO: 8014) |
| TRCBC2 binder | TRBC2 -binding, | QVQLVQSGAEVKKPGSSVKVSCKASPRGFYGYHMHWVRQA |
| BKM0312 VH-4 - | Heavy Chain | PGQGLEWMGFINPYNNHIQYNERFRGRVTITSDESTTTAYME |
| SEQ ID NO: 8303 | | LSSLRSEDTAVYYCALGAGKWGDGAYRFFDFWGQGTLVTV |
| | | SS (SEQ ID NO: 8303) |
| TRCBC2 binder | TRBC2 -binding, | DVVMTQSPLSLPVTLGQPASISCRSSKNLVHSNGRTYLQWY |
| BKM0312 VL-4 - | Light Chain | QQRPGQSPRLLIYRVSNRFPGVPDRFSGSGSGTDFTLKISRVE |
| SEQ ID NO: 8014 | | AEDVGVYFCSQSTREPYTFGGGTKVEIK (SEQ ID NO: 8014) |

TABLE 14
Heavy Chain (HC) and Light Chain (LC) CDR sequences of TRBC2 from domains listed
in Table 13.

| HC CDR1 | HC CDR2 | HC CDR3 |
|---|---|---|
| PRGFYGYHMH (SEQ ID NO: | FINPYTNDIQYNERFRG (SEQ | GNGKWGDGAYRFFDL (SEQ |
| 8272) per ABM numbering system / | ID NO: 8260) | ID NO: 8227) |
| or, {GYHMH, SEQ ID NO: 7401 | | |
| per Kabat numbering system} | | |
| PRGFYGYHMH (SEQ ID NO: | FINPYNNHIQYNERFRG (SEQ | GNGKWGDGAYRFFDF |
| 8272){GYHMH, SEQ ID NO: | ID NO: 8044) | (SEQ ID NO: 7403) |
| 7401 per Kabat numbering system} | | |
| PRGFYGYHMH (SEQ ID NO: | FINPYNNHIQYNERFRG (SEQ | GEGKWGDGAYRFFDF |
| 8272){GYHMH, SEQ ID NO: | ID NO: 8044) | (SEQ ID NO: 8046) |
| 7401 per Kabat numbering system} | | |
| PRGFYGYHMH (SEQ ID NO: | FINPYNNHIQYNERFRG (SEQ | GAGKWGDGAYRFFDF |
| 8272){or, GYHMH, SEQ ID NO: | ID NO: 8044) | (SEQ ID NO: 8047) |
| 7401 per Kabat numbering system} | | |
| LC CDR1 | LC CDR2 | LC CDR3 |
| RSSENLVHSNGRTYLQ (SEQ | RVSNRFP (SEQ ID NO: 224) | SQSSLEPYT (SEQ ID NO: 8050) |
| ID NO: 8048) | | |
| RSSKNLVHSNGRTYLQ (SEQ | RVSNRFP (SEQ ID NO: 224) | SQSTREPYT (SEQ ID NO: 8052) |
| ID NO: 8051) | | |

TABLE 18
Exemplary Fc modifications

| Modification | Altered |
|---|---|
| or mutation | effector function |
| Leu235Glu | ADCC; |
| Leu234Ala/Leu235Ala | ADCC; ADCP; |
| (LALA) | CDC |
| Ser228Pro/Leu235Glu | |
| Leu234Ala/Leu235Ala/ | ADCP |
| Pro329Gly | |
| Pro331Ser/Leu234Glu/ | CDC |
| Leu235Phe | |
| Asp265 Ala | ADCC, ADCP |
| Gly237Ala | ADCP |
| Glu318Ala | ADCP |
| Glu233Pro | |
| Gly236Arg/Leu328Arg | ADCC |
| His268Gln/Val309Leu/ | ADCC; ADCP; |
| Ala330Ser/Pro331Ser | CDC |
| Val234Ala/Gly237Ala/ | ADCC; ADCP; |
| Pro238Ser/His268Ala/ | CDC |
| Val309Leu/Ala330Ser/ | |
| Pro331Ser | |
| Leu234Ala/L235Ala/ | ADCC; CDC |
| Gly237Ala/P238Ser/ | |
| His268Ala/Ala330Ser/ | |
| Pro331Ser | |
| Ala330Leu | CDC |
| Asp270Ala | CDC |
| Lys322Ala | CDC |
| Pro329Ala | CDC |
| Pro331Ala | CDC |
| Val264Ala | CDC |
| High mannose | CDC |
| glycosylation | |
| Phe241 Ala | CDC |
| Asn297Ala or Gly or | ADCC; ADCP; |
| Gln | CDC |
| S228P/Phe234Ala/ | ADCC; CDC |
| Leu235Ala | |

TABLE 15
TRBC2 binding heavy chain and light chain pairs for scFv. In each sequence the CDR1 is
marked in bold font underlined (according to combined Kabat and Chothia [ABM] numbering
system), the CDR2 is marked in italicized font (Kabat numbering system), and the CDR3 (Kabat
numbering system) is marked in bold and italicized font that is underlined.

| Name | Sequence |
|---|---|
| L1L2P3- | QVQLVQSGAEVKKPGASVKVSCKASRSGFHGYAMHWVRQAPGQGLEWMGFINPY |
| A10 | NDDIQYNQKFQGRVTITADESTSTAYMELSSLRSEDTAVYYCARGNGKWGDGAYRF |
| VH | FDFWGQGTLVTVSS (SEQ ID NO: 8059) |
| L1L2P3- | DVVMTQSPLSLPVTPGEPASISCRSSQNLVHSNGRTYLQWYLQKPGQSPQLLIYRVS |
| A10 VL | NRFPGVPDRFSGSGSGTDFTLKISRVEAEDVGVYFCSQSTHVPYTFGGGTKVEIK |
| | (SEQ ID NO: 8060) |
| L1L2P3- | QVQLVQSGAEVKKPGSSVKVSCKASPRGFYGYHMHWVRQAPGQGLEWMGYINPY |
| A12 VH | NRDIKYNQKFQGRVTITADKSTTTAYMELSSLRSEDTAVYYCARGNGKWGDGAYRF |
| | FDFWGQGTLVTVSS (SEQ ID NO: 8061) |
| L1L2P3- | DVVMTQSPLSLPVTPGEPASISCRSSQNLVHSNGRTYLQWYLQKPGQSPQLLIYRVS |
| A12 VL | NRFPGVPDRFSGSGSGTDFTLKISRVEAEDVGVYFCSQSTHVPYTFGGGTKVEIK |
| | (SEQ ID NO: 7419) |
| L1L2P3- | QVQLVQSGAEVKKPGSSVKVSCKASPRGFHGYHMHWVRQAPGQGLEWMGYINPY |
| A2 VH | TGDIKYNERFRGRVTITSDESTTTAYMELSSLRSEDTAVYYCARGNGKWGDGAYRFF |
| | DLWGQGTLVTVSS (SEQ ID NO: 8063) |
| L1L2P3- | DVVMTQSPLSLPVTLGQPASISCRSSENLVHSNGRTYLQWYQQRPGQSPRLLIYRVS |
| A2 VL | NRFPGVPDRFSGSGSGTDFTLKISRVEAEDVGVYFCSQSTLEPFTFGGGTKVEIK |
| | (SEQ ID NO: 8064) |
| L1L2P3- | QVQLVQSGAEVKKPGSSVKVSCKASPRGFYGYHMHWVRQAPGQGLEWMGFINPY |
| A5 VH | NDDIQYNERFRGRVTITSDESTTTAYMELSSLRSEDTAVYYCALGNGKWGDGAYRFF |
| | DLWGQGTLVTVSS (SEQ ID NO: 8065) |
| L1L2P3- | DVVMTQSPLSLPVTLGQPASISCRSSQNLVHSNGRTYLQWYQQRPGQSPRLLIYRVS |
| A5 VL | NRFPGVPDRFSGSGSGTDFTLKISRVEAEDVGVYFCSQSSLLPYTFGGGTKVEIK |
| | (SEQ ID NO: 8066) |
| L1L2P3- | QVQLVQSGAEVKKPGSSVKVSCKASPRGFYGYHMHWVRQAPGQGLEWMGFINPY |
| A7 VH | NDDIQYNQKFQGRVTITADKSTTTAYMELSSLRSEDTAVYYCARGNGKWGDGAYRF |
| | FDFWGQGTLVTVSS (SEQ ID NO: 8067) |
| L1L2P3- | DVVMTQSPLSLPVTPGEPASISCRSSQNLVHSNGRTYLQWYLQKPGQSPQLLIYRVS |
| A7 VL | NRFPGVPDRFSGSGSGTDFTLKISRVEAEDVGVYFCSQSTHVPYTFGGGTKVEIK |
| | (SEQ ID NO: 8068) |
| L1L2P3- | QVQLVQSGAEVKKPGSSVKVSCKASPRGFYGYHMHWVRQAPGQGLEWMGFINPY |
| A5 VH | NDDIQYNERFRGRVTITSDESTTTAYMELSSLRSEDTAVYYCALGNGKWGDGAYRFF |
| | DLWGQGTLVTVSS (SEQ ID NO: 8069) |
| L1L2P3- | DVVMTQSPLSLPVTLGQPASISCRSSQNLVHSNGRTYLQWYQQRPGQSPRLLIYRVS |
| A5 VL | NRFPGVPDRFSGSGSGTDFTLKISRVEAEDVGVYFCSQSSLLPYTFGGGTKVEIK |
| | (SEQ ID NO: 8070) |
| L1L2P3- | QVQLVQSGAEVKKPGSSVKVSCKASPRGFYGYHMHWVRQAPGQGLEWMGFINPY |
| A7 VH | NDDIQYNQKFQGRVTITADKSTTTAYMELSSLRSEDTAVYYCARGNGKWGDGAYRF |
| | FDFWGQGTLVTVSS (SEQ ID NO: 8071) |
| L1L2P3- | DVVMTQSPLSLPVTPGEPASISCRSSQNLVHSNGRTYLQWYLQKPGQSPQLLIYRVS |
| A7 VL | NRFPGVPDRFSGSGSGTDFTLKISRVEAEDVGVYFCSQSTHVPYTFGGGTKVEIK |
| | (SEQ ID NO: 7419) |
| L1L2P3- | QVQLVQSGAEVKKPGASVKVSCKASRSGFHGYAMHWVRQAPGQGLEWMGFINPY |
| A9 VH | NDDIQYNQKFQGRVTITADESTSTAYMELSSLRSEDTAVYYCARGNGKWGDGAYRF |
| | FDLWGQGTLVTVSS (SEQ ID NO: 8073) |
| L1L2P3- | DVVMTQSPLSLPVTLGQPASISCRSSQNLVHSNARTYLQWYQQRPGQSPRLLIYRVS |
| A9 VL | NRFPGVPDRFSGSGSGTDFTLKISRVEAEDVGVYFCSQSTHVPYTFGGGTKVEIK |
| | (SEQ ID NO: 8074) |
| L1L2P3- | QVQLVQSGAEVKKPGSSVKVSCKASPRGFHGYHMHWVRQAPGQGLEWMGSINPY |
| B1 VH | TRHIQYNERFRGRVTITSDESTTTAYMELSSLRSEDTAVYYCARGNGKWGDGAYRFF |
| | DFWGQGTLVTVSS (SEQ ID NO: 8075) |
| L1L2P3- | DVVMTQSPLSLPVTLGQPASISCRSSQNLVHSNGRTYLQWYQQRPGQSPRLLIYRVS |
| B1 VL | NRFPGVPDRFSGSGSGTDFTLKISRVEAEDVGVYFCSQSSYVPFTFGGGTKVEIK |
| | (SEQ ID NO: 8076) |
| L1L2P3- | QVQLVQSGAEVKKPGSSVKVSCKASPRGFYGYHMHWVRQAPGQGLEWMGTINPY |
| B11 VH | NRDIQYNQKFQGRVTITADKSTSTAYMELSSLRSEDTAVYYCARGNGKWGDGAYRF |
| | FDLWGQGTLVTVSS (SEQ ID NO: 8077) |
| L1L2P3- | DVVMTQSPLSLPVTPGEPASISCRSSQNLVHSNGRTYLQWYLQKPGQSPQLLIYRVS |
| B11 VL | NRFPGVPDRFSGSGSGTDFTLKISRVEAEDVGVYFCSQSTHVPYTFGGGTKVEIK |
| | (SEQ ID NO: 8078) |
| L1L2P3- | QVQLVQSGAEVKKPGSSVKVSCKASPRGFYGYHMHWVRQAPGQGLEWMGTINPY |
| B12VH | NRDIQYNQKFQGRVTITADKSTSTAYMELSSLRSEDTAVYYCARGNGKWGDGAYRF |
| | FDFWGQGTLVTVSS (SEQ ID NO: 8079) |
| L1L2P3- | DVVMTQSPLSLPVTPGEPASISCRSSQNLVHSNGRTYLHWYLQKPGQSPQLLIYRVS |
| B12 | NRFPGVPDRFSGSGSGTDFTLKISRVEAEDVGVYFCSQSTHVPYTFGGGTKVEIK |
| VL | (SEQ ID NO: 8080) |
| L1L2P3- | QVQLVQSGAEVKKPGSSVKVSCKASPRGFYGYHMHWVRQAPGQGLEWMGYINPY |
| B2 | TNEIKYNERFRGRVTITSDESTTTAYMELSSLRSEDTAVYYCARGNGKWGDGAYRFF |
| | DLWGQGTLVTVSS (SEQ ID NO: 8081) |
| VL | DVVMTQSPLSLPVTLGQPASISCRSSKNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8699) |
| L1L2P3- | QVQLVQSGAEVKKPGSSVKVSCKASPRGFYGYHMHWVRQAPGQGLEWMGFINPY |
| B3 VH | NDDIQYNQKFQGRVTITADESTSTAYMELSSLRSEDTAVYYCARGNGKWGDGAYRF |
| | FDFWGQGTLVTVSS (SEQ ID NO: 8082) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK (SEQ |
| | ID NO: 8083) |
| L1L2P3- | QVQLVQSGAEVKKPGSSVKVSCKASPRGFYGYHMHWVRQAPGQGLEWMGTINPY |
| B4 VH | NAEIKYNQKFQGRVTITADKSTSTAYMELSSLRSEDTAVYYCARGNGKWGDGAYRFF |
| | DFWGQGTLVTVSS (SEQ ID NO: 8084) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYYCSQSTYEPFTFGGGTKVEIK |
| | (SEQ ID NO: 8085) |
| L1L2P3- | QVQLVQSGAEVKKPGSSVKVSCKASPRGFYGYHMHWVRQAPGQGLEWMGFINPY |
| B5VH | NRDIKYNERFRGRVTITSDKSTTTAYMELSSLRSEDTAVYYCAMGNGKWGDGAYRFF |
| | DLWGQGTLVTVSS (SEQ ID NO: 8086) |
| VL | DVVMTQSPLSLPVTLGQPASISCRSSKNLVHSNGRTYLQWYQQRPGQSPRLLIYRVS |
| | NRFPGVPDRFSGSGSGTDFTLKISRVEAEDVGVYFCSQSSHIPYTFGGGTKVEIK (SEQ |
| | ID NO: 8087) |
| L1L2P3- | QVQLVQSGAEVKKPGSSVKVSCKASPRGFYGYHMHWVRQAPGQGLEWMGAINPY |
| B7VH | TNDIKYNERFRGRVTITSDKSTTTAYMELSSLRSEDTAVYYCARGNGKWGDGAYRFF |
| | DLWGQGTLVTVSS (SEQ ID NO: 8088) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8089) |
| L1L2P3- | QVQLVHSGAEVKKPGSSVKVSCKASPRGFYGYHMHWVRQAPGQGLEWMGTINPY |
| B8VH | NRHIKYNQKFQGRVTITADKSTTTAYMELSSLRSEDTAVYYCARGNGKWGDGAYRF |
| | FDFWGQGTLVTVSS (SEQ ID NO: 8090) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 7419) |
| L1L2P3- | QVQLVQSGAEVKKPGASVKVSCKASPRGFHGYHMHWVRQAPGQGLEWMGSINPY |
| C1VH | TDDIQYNERFRGRVTITSDESTTTAYMELSSLRSEDTAVYYCARGNGKWGDGAYRFF |
| | DFWGQGTLVTVSS (SEQ ID NO: 8092) |
| VL | DVVMTQSPLSLPVTLGQPASISCRSSENLVHSNGRTYLQWYQQRPGQSPRLLIYRVSN |
| | RFPGVPDRFSGSGSGTDFTLKISRVEAEDVGVYFCSQSTHVPYTFGGGTKVEIK (SEQ |
| | ID NO: 8093) |
| L1L2P3- | QVQLVQSGAEVKKPGSSVKVSCKASPRGFYGYHMHWVRQAPGQGLEWMGAINPY |
| C10VH | TNHIQYNERFRGRVTITSDESTTTAYMELSSLRSEDTAVYYCARGNGKWGDGAYRFF |
| | DLWGQGTLVTVSS (SEQ ID NO: 8094) |
| VL | DVVMTQSPLSLPVTLGQPASISCRSSQNLVHSNGRTYLQWYQQRPGQSPRLLIYRVS |
| | NRFPGVPDRFSGSGSGTDFTLKISRVEAEDVGVYFCSQSTHVPYTFGGGTKVEIK |
| | (SEQ ID NO: 8095) |
| L1L2P3- | QVQLVQSGAEVKKPGSSVKVSCKASPRGFYGYHMHWVRQAPGQGLEWMGFINPY |
| C4 VH | NDDIQYNQKFQGRVTITADKSTSTAYMELSSLRSEDTAVYYCARGNGKWGDGAYRF |
| | FDFWGQGTLVTVSS (SEQ ID NO: 8096) |
| VL | DVVMTQSPLSLPVTLGQPASISCRSSQNLVHSNARTYLQWYQQRPGQSPRLLIYRVSN |
| | RFPGVPDRFSGSGSGTDFTLKISRVEAEDVGVYYCSQSTYIPYTFGGGTKVEIK (SEQ |
| | ID NO: 8097) |
| L1L2P3- | QVQLVQSGAEVKKPGSSVKVSCKASPRGFHGYHMHWVRQAPGQGLEWMGFINPY |
| C5 | TRAIKYNERFRGRVTITSDKSTTTAYMELSSLRSEDTAVYYCARGNGKWGDGAYRFF |
| VH | DLWGQGTLVTVSS (SEQ ID NO: 8098) |
| VL | DVVMTQSPLSLPVTLGQPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8099) |
| L1L2P3- | QVQLVQSGAEVKKPGSSVKVSCKASPRGFYGYHMHWVRQAPGQGLEWMGAINPY |
| C7 | TRAIKYNERFRGRVTITSDESTTTAYMELSSLRSEDTAVYYCARGNGKWGDGAYRFF |
| VH | DLWGQGTLVTVSS (SEQ ID NO: 8100) |
| VL | DVVMTQSPLSLPVTLGQPASISCRSSQNLVHSNGRTYLQWYQQRPGQSPRLLIYRVS |
| | NRFPGVPDRFSGSGSGTDFTLKISRVEAEDVGVYFCSQSTYEPYTFGGGTKVEIK |
| | (SEQ ID NO: 8101) |
| L1L2P3- | QVQLVQSGAEVKKPGSSVKVSCKASPRGFYGYHMHWVRQAPGQGLEWMGTINPY |
| C9 | NGDIQYNERFRGRVTMTSDKSTTTAYMELSSLRSEDTAVYYCARGNGKWGDGAYRF |
| VH | FDFWGQGTLVTVSS (SEQ ID NO: 8102) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 7419) |
| L1L2P3- | QVQLVQSGAEVKKPGSSVKVSCKASPRGFYGYHMHWVRQAPGQGLEWMGAINPY |
| D10 | NTDIKYNERFRGRVTITSDESTTTAYMELSSLRSEDTAVYYCARGNGKWGDGAYRFF |
| VH | DLWGQGTLVTVSS (SEQ ID NO: 8104) |
| VL | DVVMTQSPLSLPVTLGQPASISCRSSQNLVHSNGRTYLQWYQQRPGQSPRLLIYRVS |
| | NRFPGVPDRFSGSGSGTDFTLKISRVEAEDVGVYFCSQSTHVPYTFGGGTKVEIK |
| | (SEQ ID NO: 8105) |
| L1L2P3- | QVQLVQSGAEVKKPGSSVKVSCKASPRGFYGYHMHWVRQAPGQGLEWMGYINPY |
| D12 | NGAIKYNQKFQGRVTITADESTSTAYMELSSLRSEDTAVYYCARGNGKWGDGAYRFF |
| VH | DLWGQGTLVTVSS (SEQ ID NO: 8106) |
| VL | DVVMTQSPLSLPVTLGQPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8107) |
| L1L2P3- | QVQLVQSGAEVKKPGSSVKVSCKASPRGFYGYHMHWVRQAPGQGLEWMGAINPY |
| E10 | NDDIQSNERFRGRVTITSDKSTTTAYMELSSLRSEDTAVYYCARGNGKWGDGAYRFF |
| VH | DLWGQGTLVTVSS (SEQ ID NO: 8108) |
| VL | DVVMTQSPLSLPVTLGQPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8109) |
| L1L2P3- | QVQLVQSGAEVKKPGSSVKVSCKASPRGFYGYHMHWVRQAPGQGLEWMGFINPY |
| E12 | NRAIQYNQKFQGRVTITADKSTTTAYMELSSLRSEDTAVYYCARGNGKWGDGAYRF |
| VH | FDFWGQGTLVTVSS (SEQ ID NO: 8110) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 7419) |
| L1L2P3- | QVQLVQSGAEVKKPGASVKVSCKASPRGFHGYHMHWVRQAPGQGLEWMGFINPY |
| E6 | TNEIQYNERFRGRVTITSDESTTTAYMELSSLRSEDTAVYYCARGNGKWGDGAYRFF |
| VH | DLWGQGTLVTVSS (SEQ ID NO: 8113) |
| VL | DVVMTQSPLSLPVTLGQPASISCRSSQNLVHSNGRTYLQWYQQRPGQSPRLLIYRVS |
| | NRFPGVPDRFSGSGSGTDFTLKISRVEAEDVGVYFCSQSTHVPYTFGGGTKVEIK |
| | (SEQ ID NO: 8114) |
| L1L2P3- | QVQLVQSGAEVKKPGSSVKVSCKASPRGFYGYHMHWVRQAPGQGLEWMGYINPY |
| E9 | NHDIQYNQKFQGRVTITADKSTSTAYMELSSLRSEDTAVYYCARGNGKWGDGAYRF |
| VH | FDFWGQGTLVTVSS (SEQ ID NO: 8115) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 7419) |
| L1L2P3- | QVQLVQSGAEVKKPGSSVKVSCKASPRGFYGYHMHWVRQAPGQGLEWMGSINPY |
| F1 | THDIQYNERFRGRVTITSDESTTTAYMELSSLRSEDTAVYYCARGNGKWGDGAYRFF |
| aTRBC2e | DLWGQGTLVTVSS (SEQ ID NO: 8117) |
| 31 VH | |
| VL | DVVMTQSPLSLPVTLGQPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8118) |
| L1L2P3- | QVQLVQSGAEVKKPGSSVKVSCKASPRGFYGYHMHWVRQAPGQGLEWMGYINPY |
| F11 | KNAIQYNQKFQGRVTITADKSTTTAYMELSSLRSEDTAVYYCARGNGKWGDGAYRF |
| VH | FDFWGQGTLVTVSS (SEQ ID NO: 8119) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 7419) |
| L1L2P3- | QVQLVQSGAEVKKPGSSVKVSCKASPRGFYGYHMHWVRQAPGQGLEWMGAINPY |
| F2 | NTDIQYNERFRGRVTITSDKSTTTAYMELSSLRSEDTAVYYCARGNGKWGDGAYRFF |
| VH | DFWGQGTLVTVSS (SEQ ID NO: 8121) |
| VL | DVVMTQSPLSLPVTLGQPASISCRSSENLVHSNGRTYLQWYQQRPGQSPRLLIYRVSN |
| | RFPGVPDRFSGSGSGTDFTLKISRVEAEDVGVYFCSQSSHIPYTFGGGTKVEIK (SEQ |
| | ID NO: 8122) |
| L1L2P3- | QVQLVQSGAEVKKPGSSVKVSCKASPRGFYGYHMHWVRQAPGQGLEWMGSINPY |
| F6 | NGDIQYNERFRGRVTITSDKSTTTAYMELSSLRSEDTAVYYCARGNGKWGDGAYRFF |
| VH | DFWGQGTLVTVSS (SEQ ID NO: 8123) |
| VL | DVVMTQSPLSLPVTLGQPASISCRSSQNLVHSNGRTYLQWYQQRPGQSPRLLIYRVS |
| | NRFPGVPDRFSGSGSGTDFTLKISRVEAEDVGVYFCSQSTHLPYTFGGGTKVEIK |
| | (SEQ ID NO: 8124) |
| L1L2P3- | QVQLVQSGAEVKKPGSSVKVSCKASPRGFYGYHMHWVRQAPGQGLEWMGTINPY |
| G10 | NHDAQYNERFRGRVTITSDKSTTTAYMELSSLRSEDTAVYYCARGNGKWGDGAYRF |
| VH | FDFWGQGTLVTVSS (SEQ ID NO: 8125) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 7419) |
| L1L2P3- | QVQLVQSGAEVKKPGSSVKVSCKASPRGFYGYHMHWVRQAPGQGLEWMGFINPY |
| G11 | NHAIKYNQKFQGRVTITADKSTSTAYMELSSLRSEDTAVYYCARGNGKWGDGAYRF |
| VH | FDFWGQGTLVTVSS (SEQ ID NO: 8127) |
| VL | DVVMTQSPLSLPVTPGEPASISCRSSQNLVHSNGRTYLHWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8128) |
| L1L2P3- | QVQLVQSGAEVKKPGSSVKVSCKASPRGFYGYHMHWVRQAPGQGLEWMGYINPY |
| G3 | NNAIQYNQKFQGRVTMTSDKSTTTAYMELSSLRSEDTAVYYCARGNGKWGDGAYR |
| VH | FFDFWGQGTLVTVSS (SEQ ID NO: 8129) |
| | DVVMTQSPLSLPVTLGQPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8130) |
| L1L2P3- | QVQLVQSGAEVKKPGSSVKVSCKASPRGFYGYHMHWVRQAPGQGLEWMGFINPY |
| G6 | TNDIQYNERFRGRVTITSDKSTTTAYMELSSLRSEDTAVYYCAMGNGKWGDGAYRF |
| VH | FDLWGQGTLVTVSS (SEQ ID NO: 8131) |
| VL | DVVMTQSPLSLPVTLGQPASISCRSSQNLVHSNGRTYLQWYQQRPGQSPRLLIYRVS |
| | NRFPGVPDRFSGSGSGTDFTLKISRVEAEDVGVYFCSQSTHVPYTFGGGTKVEIK |
| | (SEQ ID NO: 8132) |
| L1L2P3- | QVQLVQSGAEVKKPGSSVKVSCKASPRGFYGYHMHWVRQAPGQGLEWMGAINPY |
| G9 | TNEIKYNERFRGRVTITSDESTTTAYMELSSLRSEDTAVYYCARGNGKWGDGAYRFF |
| VH | DFWGQGTLVTVSS (SEQ ID NO: 8133) |
| VL | DVVMTQSPLSLPVTLGQPASISCRSSQNLVHSNGRTYLQWYQQRPGQSPRLLIYRVS |
| | NRFPGVPDRFSGSGSGTDFTLKISRVEAEDVGVYFCSQSTHLPYTFGGGTKVEIK |
| | (SEQ ID NO: 8134) |
| L1L2P3- | QVQLVQSGAEVKKPGSSVKVSCKASPRGFYGYHMHWVRQAPGQGLEWMGFINPY |
| H11 | NDDIQYNQKFQGRVTITADKSTSTAYMELSSLRSEDTAVYYCARGNGKWGDGAYRF |
| VH | FDFWGQGTLVTVSS (SEQ ID NO: 8135) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8136) |
| L1L2P3- | QVQLVQSGAEVKKPGSSVKVSCKASPRGFYGYHMHWVRQAPGQGLEWMGAINPY |
| H2 | NDDIQYNERFRGRVTITSDESTTTAYMELSSLRSEDTAVYYCALGNGKWGDGAYRFF |
| VH | DFWGQGTLVTVSS (SEQ ID NO: 8137) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8138) |
| L1L2P3- | QVQLVQSGAEVKKPGASVKVSCKASPRGFHGYHMHWVRQAPGQGLEWMGYINPY |
| H3 | NNDIKYNQKFQGRVTITADESTSTAYMELSSLRSEDTAVYYCARGNGKWGDGAYRFF |
| VH | DFWGQGTLVTVSS (SEQ ID NO: 8139) |
| VL | DVVMTQSPLSLPVTLGQPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8140) |
| L1L2P3- | QVQLVQSGAEVKKPGSSVKVSCKASPRGFHGYHMHWVRQAPGQGLEWMGFINPY |
| H7 | TNDIQYNERFRGRVTITSDESTTTAYMELSSLRSEDTAVYYCALGNGKWGDGAYRFF |
| | DLWGQGTLVTVSS (SEQ ID NO: 8141) |
| VL | DVVMTQSPLSLPVTLGQPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8142) |
| L1L2P3- | QVQLVQSGAEVKKPGSSVKVSCKASPRGFYGYHMHWVRQAPGQGLEWMGTINPY |
| H9 | KHHIKYNERFRGRVTITSDESTTTAYMELSSLRSEDTAVYYCARGNGKWGDGAYRFF |
| VH | DLWGQGTLVTVSS (SEQ ID NO: 8143) |
| VL | DVVMTQSPLSLPVTLGQPASISCRSSENLVHSNGRTYLQWYQQRPGQSPRLLIYRVSN |
| | RFPGVPDRFSGSGSGTDFTLKISRVEAEDVGVYFCSQSTQEPYTFGGGTKVEIK (SEQ |
| | ID NO: 8144) |
| L1P1-A2 | QVQLVQSGAEVKKPGSSVKVSCKASPRGFYGYHMHWVRQAPGQGLEWMGTINPY |
| VH | NGDIQYNERFRGRVTITSDESTTTAYMELSSLRSEDTAVYYCARGNGKWGDGAYRFF |
| | DFWGQGTLVTVSS (SEQ ID NO: 8145) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8146) |
| L1P1-A3 | QVQLVQSGAEVKKPGSSVKVSCKASPRGFYGYHMHWVRQAPGQGLEWMGAINPY |
| VH | NDDIKYNERFRGRVTITSDKSTTTAYMELSSLRSEDTAVYYCALGNGKWGDGAYRFF |
| | DFWGQGTLVTVSS (SEQ ID NO: 8147) |
| VL | DVVMTQSPLSLPVTLGQPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8148) |
| L1P1-A4 | QVQLVQSGAEVKKPGSSVKVSCKASPRGFYGYHMHWVRQAPGQGLEWMGYINPY |
| VH | THEIKYNERFRGRVTITSDESTTTAYMELSSLRSEDTAVYYCARGNGKWGDGAYRFF |
| | DFWGQGTLVTVSS (SEQ ID NO: 8149) |
| VL | DVVMTQSPLSLPVTLGQPASISCRSSKNLVHSNARTYLQWYQQRPGQSPRLLIYRVS |
| | NRFPGVPDRFSGSGSGTDFTLKISRVEAEDVGVYFCSQSTHVPYTFGGGTKVEIK |
| | (SEQ ID NO: 8150) |
| L1P1-A5 | QVQLVQSGAEVKKPGSSVKVSCKASPRGFYGYHMHWVRQAPGQGLEWMGFINPY |
| VH | KDDIKYNERFRGRVTITSDKSTTTAYMELSSLRSEDTAVYYCALGNGKWGDGAYRFF |
| | DFWGQGTLVTVSS (SEQ ID NO: 8151) |
| VL | DVVMTQSPLSLPVTLGQPASISCRSSKNLVHSNGRTYLQWYQQRPGQSPRLLIYRVS |
| | NRFPGVPDRFSGSGSGTDFTLKISRVEAEDVGVYFCSQSSLVPYTFGGGTKVEIK |
| | (SEQ ID NO: 8152) |
| L1P1-A6 | QVQLVQSGAEVKKPGSSVKVSCKASPRGFYGYHMHWVRQAPGQGLEWMGAINPY |
| VH | NDDIQSNERFRGRVTITSDKSTTTAYMELSSLRSEDTAVYYCARGNGKWGDGAYRFF |
| | DFWGQGTLVTVSS (SEQ ID NO: 8153) |
| VL | DVVMTQSPLSLPVTPGEPASISCRSSQNLVHSNGRTYLHWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8154) |
| L1P1-A8 | QVQLVQSGAEVKKPGSSVKVSCKASPRGFYGYHMHWVRQAPGQGLEWMGAINPY |
| VH | NDDIKYNQKFQGRVTITADESTTTAYMELSSLRSEDTAVYYCARGNGKWGDGAYRF |
| | FDFWGQGTLVTVSS (SEQ ID NO: 8155) |
| VL | DVVMTQSPLSLPVTPGEPASISCRSSQNLVHSNGRTYLHWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8156) |
| L1P1-B1 | QVQLVQSGAEVKKPGSSLKVSCKASPRGFHGYHMHWVRQAPGQGLEWMGAINPYN |
| VH | RDIKYNERFRGRVTITSDESTTTAYMELSSLRSEDTAVYYCARGNGKWGDGAYRFFD |
| | LWGQGTLVTVSS (SEQ ID NO: 8157) |
| VL | DVVMTQSPLSLPVTLGQPASISCRSSKNLVHSNGRTYLQWYQQRPGQSPRLLIYRVS |
| | NRFPGVPDRFSGSGSGTDFTLKISRVEAEDVGVYFCSQSTHVPYTFGGGTKVEIK |
| | (SEQ ID NO: 8158) |
| L1P1-B6 | QVQLVQSGAEVKKPGSSVKVSCKASPRGFYGYHMHWVRQAPGQGLEWMGAINPY |
| VH | NGDIKYNERFRGRVTITSDKSTTTAYMELSSLRSEDTAVYYCARGNGKWGDGAYRFF |
| | DFWGQGTLVTVSS (SEQ ID NO: 8159) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8160) |
| L1P1-B8 | QVQLVQSGAEVKKPGSSVKVSCKASPRGFYGYHMHWVRQAPGQGLEWMGFINPY |
| VH | NNHIQYNERFRGRVTITSDESTTTAYMELSSLRSEDTAVYYCALGNGKWGDGAYRFF |
| | DFWGQGTLVTVSS (SEQ ID NO: 8161) |
| VL | DVVMTQSPLSLPVTLGQPASISCRSSKNLVHSNGRTYLQWYQQRPGQSPRLLIYRVS |
| | NRFPGVPDRFSGSGSGTDFTLKISRVEAEDVGVYFCSQSTREPYTFGGGTKVEIK |
| | (SEQ ID NO: 8162) |
| L1P1-D1 | QVQLVQSGAEVKKPGSSVKVSCKASPRGFYGYHMHWVRQAPGQGLEWMGYINPY |
| VH | TRDIKYNERFRGRVTITSDESTTTAYMELSSLRSEDTAVYYCARGNGKWGDGAYRFF |
| | DFWGQGTLVTVSS (SEQ ID NO: 8163) |
| VL | DVVMTQSPLSLPVTLGQPASISCRSSQNLVHSNGRTYLQWYQQRPGQSPRLLIYRVS |
| | NRFPGVPDRFSGSGSGTDFTLKISRVEAEDVGVYFCSQSTQMPYTFGGGTKVEIK |
| | (SEQ ID NO: 8164) |
| L1P1-D3 | QVQLVQSGAEVKKPGSSVKVSCKASPRGFYGYHMHWVRQAPGQGLEWMGTINPY |
| VH | NTDIKYNERFRGRVTITSDKSTTTAYMELSSLRSEDTAVYYCARGNGKWGDGAYRFF |
| | DLWGQGTLVTVSS (SEQ ID NO: 8165) |
| VL | DVVMTQSPLSLPVTLGQPASISCRSSQNLVHSNARTYLQWYQQRPGQSPRLLIYRVS |
| | NRFPGVPDRFSGSGSGTDFTLKISRVEAEDVGVYFCSQSTYEPYTFGGGTKVEIK |
| | (SEQ ID NO: 8166) |
| L1P1-D6 | QVQLVQSGAEVKKPGSSVKVSCKASPRGFYGYHMHWVRQAPGQGLEWMGTINPY |
| VH | NNDIQYNERFRGRVTITSDKSTTTAYMELSSLRSEDTAVYYCARGNGKWGDGAYRFF |
| | DFWGQGTLVTVSS (SEQ ID NO: 8167) |
| VL | DVVMTQSPLSLPVTLGQPASISCRSSQNLVHSNGRTYLHWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8168) |
| L1P1-E1 | QVQLVQSGAEVKKPGSSVKVSCKASPRGFYGYHMHWVRQAPGQGLEWMGYINPY |
| VH | NGNIQYNERFRGRVTITSDESTTTAYMELSSLRSEDTAVYYCARGNGKWGDGAYRFF |
| | DFWGQGTLVTVSS (SEQ ID NO: 8169) |
| VL | DVVMTQSPLSLPVTLGQPASISCRSSKNLVHSNGRTYLQWYQQRPGQSPRLLIYRVS |
| | NRFPGVPDRFSGSGSGTDFTLKISRVEAEDVGVYFCSQSSQEPYTFGGGTKVEIK |
| | (SEQ ID NO: 8170) |
| L1P1-E2 | QVQLVQSGAEVKKPGSSVKVSCKASPRGFYGYHMHWVRQAPGQGLEWMGAINPY |
| VH | TNEIQYNERFRGRVTITSDESTTTAYMELSSLRSEDTAVYYCARGNGKWGDGAYRFF |
| | DFWGQGTLVTVSS (SEQ ID NO: 8171) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8172) |
| L1P1- | QVQLVQSGAEVKKPGASVKVSCKASPRGFYGYHMHWVRQAPGQGLEWMGSINPY |
| E6VH | NHDIKYNERFRGRVTITSDKSTTTAYMELSSLRSEDTAVYYCARGNGKWGDGAYRFF |
| | DFWGQGTLVTVSS (SEQ ID NO: 8173) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8174) |
| L1P1-F1 | QVQLVQSGAEVKKPGSSVKVSCKASPRGFYGYHMHWVRQAPGQGLEWMGFINPY |
| VH | KNEIKYNERFRGRVTITSDESTTTAYMELSSLRSEDTAVYYCARGNGKWGDGAYRFF |
| | DFWGQGTLVTVSS (SEQ ID NO: 8175) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8176) |
| L1P1-F4 | QVQLVQSGAEVKKPGSSVKVSCKASPRGFYGYHMHWVRQAPGQGLEWMGYINPY |
| VH | NNEIQYNERFRGRVTITSDESTTTAYMELSSLRSEDTAVYYCARGNGKWGDGAYRFF |
| | DFWGQGTLVTVSS (SEQ ID NO: 8177) |
| VL | DVVMTQSPLSLPVTPGEPASISCRSSQNLVHSNGRTYLHWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8178) |
| L1P1-F6 | QVQLVQSGAEVKKPGSSVKVSCKASPRGFYGYHMHWVRQAPGQGLEWMGSINPY |
| VH | NRHIQYNERFRGRVTITSDKSTTTAYMELSSLRSEDTAVYYCARGNGKWGDGAYRFF |
| | DFWGQGTLVTVSS (SEQ ID NO: 8179) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8180) |
| L1P1-F7 | QVQLVQSGAEVKKPGSSVKVSCKASPRGFYGYHMHWVRQAPGQGLEWMGSINPY |
| VH | TREIQYNERFRGRVTITSDKSTTTAYMELSSLRSEDTAVYYCARGNGKWGDGAYRFF |
| | DLWGQGTLVTVSS (SEQ ID NO: 8181) |
| VL | DVVMTQSPLSLPVTLGQPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8182) |
| L1P1-F9 | QVQLVQSGAEVKKPGSSVKVSCKASPRGFYGYHMHWVRQAPGQGLEWMGFINPY |
| VH | TNDIQYNERFRGRVTITSDESTTTAYMELSSLRSEDTAVYYCAMGNGKWGDGAYRFF |
| | DLWGQGTLVTVSS (SEQ ID NO: 8183) |
| VL | DVVMTQSPLSLPVTLGQPASISCRSSENLVHSNGRTYLQWYQQRPGQSPRLLIYRVS |
| | NRFPGVPDRFSGSGSGTDFTLKISRVEAEDVGVYFCSQSSLEPYTFGGGTKVEIK |
| | (SEQ ID NO: 8184) |
| L1P1-G9 | QVQLVQSGAEVKKPGSSVKVSCKASPRGFYGYHMHWVRQAPGQGLEWMGFINPY |
| VH | NDDIQSNERFQGRVTITADESTSTAYMELSSLRSEDTAVYYCAMGNGKWGDGAYRF |
| | FDLWGQGTLVTVSS (SEQ ID NO: 8185) |
| VL | DVVMTQSPLSLPVTLGQPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8186) |
| L1P1-H4 | QVQLVQSGAEVKKPGSSVKVSCKASPRGFYGYHMHWVRQAPGQGLEWMGSINPY |
| VH | THNIKYNERFRGRVTITSDESTTTAYMELSSLRSEDTAVYYCARGNGKWGDGAYRFF |
| | DFWGQGTLVTVSS (SEQ ID NO: 8187) |
| VL | DVVMTQSPLSLPVTLGQPASISCRSSQNLVHSNGRTYLQWYQQRPGQSPRLLIYRVS |
| | NRFPGVPDRFSGSGSGTDFTLKISRVEAEDVGVYFCSQSTHVPYTFGGGTKVEIK |
| | (SEQ ID NO: 8188) |
| L2P1-A7 | QVQLVQSGAEVKKPGSSVKVSCKASPRGFYGYHMHWVRQAPGQGLEWMGYINPY |
| VH | NHEIKYNQKFQGRVTITADKSTTTAYMELSSLRSEDTAVYYCARGNGKWGDGAYRF |
| | FDFWGQGTLVTVSS (SEQ ID NO: 8189) |
| VL | DVVMTQSPLSLPVTPGEPASISCRSSQNLVHSNGRTYLHWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8190) |
| L2P1-B12 | QVQLVQSGAEVKKPGSSVKVSCKASPRGFYGYHMHWVRQAPGQGLEWMGSINPY |
| VH | TRNIQYNQKFRGRVTMTSDKSTTTAYMELSSLRSEDTAVYYCARGNGKWGDGAYR |
| | FFDFWGQGTLVTVSS (SEQ ID NO: 8191) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8192) |
| L2P1-D7 | QVQLVQSGAEVKKPGSSVKVSCKASPRGFYGYHMHWVRQAPGQGLEWMGYINPY |
| VH | NGAIQYNQKFQGRVTITADESTSTAYMELSSLRSEDTAVYYCARGNGKWGDGAYRF |
| | FDLWGQGTLVTVSS (SEQ ID NO: 8193) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8194) |
| L2P1-D8 | QVQLVQSGAEVKKPGSSVKVSCKASPRGFYGYHMHWVRQAPGQGLEWMGTINPY |
| VH | TREIQYNQKFQGRVTITADKSTTTAYMELSSLRSEDTAVYYCARGNGKWGDGAYRFF |
| | DFWGQGTLVTVSS (SEQ ID NO: 8195) |
| VL | DVVMTQSPLSLPVTPGEPASISCRSSQNLVHSNGRTYLHWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8196) |
| L2P1-E9 | QVQLVQSGAEVKKPGSSVKVSCKASPRGFYGYAMHWVRQAPGQGLEWMGYINPY |
| VH | NNEIQYNQKFQGRVTITADKSTTTAYMELSSLRSEDTAVYYCARGNGKWGDGAYRF |
| | FDFWGQGTLVTVSS (SEQ ID NO: 8197) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8198) |
| L2P1-F12 | QVQLVQSGAEVKKPGSSVKVSCKASPRGFYGYHMHWVRQAPGQGLEWMGAINPY |
| VH | NHEIQYNQKFQGRVTITADKSTTTAYMELSSLRSEDTAVYYCARGNGKWGDGAYRF |
| | FDFWGQGTLVTVSS (SEQ ID NO: 8199) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYYCSQSTHVPYTFGGGTKVEIK |
| | (SEQ ID NO: 8200) |
| L2P1-F3 | QVQLVQSGAEVKKPGSSVKVSCKASPRGFHGYHMHWVRQAPGQGLEWMGFINPY |
| VH | NDDIQYNQKFQGRVTITADESTSTAYMELSSLRSEDTAVYYCARGNGKWGDGAYRF |
| | FDFWGQGTLVTVSS (SEQ ID NO: 8201) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8202) |
| L2P1-F5 | QVQLVQSGAEVKKPGSSVKVSCKASPRGFYGYHMHWVRQAPGQGLEWMGFINPY |
| VH | NDDIQYNQKFQGRVTITADESTSTAYMELSSLRSEDTAVYYCAMGNGKWGDGAYRF |
| | FDLWGQGTLVTVSS (SEQ ID NO: 8203) |
| aTRBC2e | DVVMTQSPLSLPVTLGQPASISCRSSQNLVHSNGRTYLQWYLQKPGQSPQLLIYRVS |
| 75 VL | NRFPGVPDRFSGSGSGTDFTLKISRVEAEDVGVYFCSQSTHVPYTFGGGTKVEIK |
| | (SEQ ID NO: 8204) |
| L2P1-H8 | QVQLVQSGAEVKKPGSSVKVSCKASPRGFYGYHMHWVRQAPGQGLEWMGFINPY |
| VH | NHAIKYNQKFQGRVTITADESTSTAYMELSSLRSEDTAVYYCARGNGKWGDGAYRFF |
| | DFWGQGTLVTVSS (SEQ ID NO: 8205) |
| VL | DVVMTQSPLSLPVTPGEPASISCRSSQNLVHSNGRTYLQWYLQKPGQSPQLLIYRVS |
| | NRFPGVPDRFSGSGSGTDFTLKISRVEAEDVGVYFCSQSTHVPYTFGGGTKVEIK |
| | (SEQ ID NO: 8206) |

TABLE 16
Exemplary antibody molecules that bind to TRBC1 AND/OR NKp30

| | | |
|---|---|---|
| SEQ | | |
| ID NO | Description | Sequence |

Anti-TRBC1-NKp30-BJM0772

| | | |
|---|---|---|
| SEQ | anti-TRBC1 | QVQLVQSGAEVKKPGSSVKVSCKASGYTFTGYVMHWVRQAPGQGLEW |
| ID | HC | MGFINPYNDDIQSNERFRGRVTITSDKSTTTAYMELSSLRSEDTAVYYCA |
| NO: | | RGAGYNFDGAYRFFDFWGQGTLVTVSSASTKGPSVFPLAPSSKSTSGGT |
| 7379 | | AALGCLVKDYFPEPVTVSWNSGALTSGVHTFPAVLQSSGLYSLSSVVTVP |
| | | SSSLGTQTYICNVNHKPSNTKVDKRVEPKSCDKTHTCPPCPAPELLGGPSV |
| | | FLFPPKPKDTLMISRTPEVTCVVVDVSHEDPEVKFNWYVDGVEVHNAKT |
| | | KPREEQYASTYRVVSVLTVLHQDWLNGKEYKCKVSNKALPAPIEKTISKA |
| | | KGQPREPQVYTLPPCREEMTKNQVSLWCLVKGFYPSDIAVEWESNGQPE |
| | | NNYKTTPPVLDSDGSFFLYSKLTVDKSRWQQGNVFSCSVMHEALHNHYT |
| | | QKSLSLSPGK |
| SEQ | anti-TRBC1 | QVQLVQSGAEVKKPGSSVKVSCKASGYTFTGYVMHWVRQAPGQGLEW |
| ID | HC | MGFINPYNDDIQSNERFRGRVTITSDKSTTTAYMELSSLRSEDTAVYYCA |
| NO: | | RGAGYNFDGAYRFFDFWGQGTLVTVSSASTKGPSVFPLAPSSKSTSGGT |
| 4675 | | AALGCLVKDYFPEPVTVSWNSGALTSGVHTFPAVLQSSGLYSLSSVVTVP |
| | | SSSLGTQTYICNVNHKPSNTKVDKRVEPKSCDKTHTCPPCPAPELLGGPSV |
| | | FLFPPKPKDTLMISRTPEVTCVVVDVSHEDPEVKFNWYVDGVEVHNAKT |
| | | KPREEQYASTYRVVSVLTVLHQDWLNGKEYKCKVSNKALPAPIEKTISKA |
| | | KGQPREPQVYTLPPCREEMTKNQVSLWCLVKGFYPSDIAVEWESNGQPE |
| | | NNYKTTPPVLDSDGSFFLYSKLTVDKSRWQQGNVFSCSVMHEALHNHYT |
| | | QKSLSLSP |
| SEQ | anti-TRBC1 | DVVMTQSPLSLPVTLGQPASISCRSSQRLVHSNGNTYLHWYQQRPGQSP |
| ID | LC | RLLIYRVSNRFPGVPDRFSGSGSGTDFTLKISRVEAEDVGVYFCSQSTHVP |
| NO: | | YTFGGGTKVEIKRTVAAPSVFIFPPSDEQLKSGTASVVCLLNNFYPREAKV |
| 7380 | | QWKVDNALQSGNSQESVTEQDSKDSTYSLSSTLTLSKADYEKHKVYACE |
| | | VTHQGLSSPVTKSFNRGEC |
| SEQ | anti-NKp30 | QIQLQESGPGLVKPSQSLSLSCSVTGFSITTTGYHWNWIRQFPGKKLEWM |
| ID | 15E1 scFv- | GYIYSSGSTSYNPSLKSRFSITRDTSKNQFFLQLNSVTTEDTATYYCARGD |
| NO: | Fc | WHYFDYWGPGTMVTVSSGGGGSGGGGSGGGGSGGGGSSFTLTQPPLVS |
| 7381 | | VAVGQVATITCSGEKLSDKYVHWYQQKPGRAPVMVIYENDRRPSGIPDQ |
| | | FSGSNSGNIASLTISKAQAGDEADYFCQFWDSTNSAVFGGGTQLTVLDKT |
| | | HTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCK |
| | | VSNKALPAPIEKTISKAKGQPREPQVCTLPPSREEMTKNQVSLSCAVKGFY |
| | | PSDIAVEWESNGQPENNYKTTPPVLDSDGSFFLVSKLTVDKSRWQQGNVF |
| | | SCSVMHEALHNRFTQKSLSLSPGK |
| SEQ | anti-NKp30 | QIQLQESGPGLVKPSQSLSLSCSVTGFSITTTGYHWNWIRQFPGKKLEWM |
| ID | 15E1 scFv- | GYIYSSGSTSYNPSLKSRFSITRDTSKNQFFLQLNSVTTEDTATYYCARGD |
| NO: | Fc | WHYFDYWGPGTMVTVSSGGGGSGGGGSGGGGSGGGGSSFTLTQPPLVS |
| 4676 | | VAVGQVATITCSGEKLSDKYVHWYQQKPGRAPVMVIYENDRRPSGIPDQ |
| | | FSGSNSGNIASLTISKAQAGDEADYFCQFWDSTNSAVFGGGTQLTVLDKT |
| | | HTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCK |
| | | VSNKALPAPIEKTISKAKGQPREPQVCTLPPSREEMTKNQVSLSCAVKGFY |
| | | PSDIAVEWESNGQPENNYKTTPPVLDSDGSFFLVSKLTVDKSRWQQGNVF |
| | | SCSVMHEALHNRFTQKSLSLSP |

Anti-TRBC1-NKp30-BJM1042

| | | |
|---|---|---|
| SEQ | anti-TRBC1 | QVQLVQSGAEVKKPGSSVKVSCKASGYTFTGYVMHWVRQAPGQGLEW |
| ID | HC | MGFIIPIFGTANYAQKFQGRVTITSDKSTTTAYMELSSLRSEDTAVYYCA |
| NO: | | RGAGYNFDGAYRFFDFWGQGTLVTVSSASTKGPSVFPLAPSSKSTSGGT |
| 7382 | | AALGCLVKDYFPEPVTVSWNSGALTSGVHTFPAVLQSSGLYSLSSVVTVP |
| | | SSSLGTQTYICNVNHKPSNTKVDKRVEPKSCDKTHTCPPCPAPELLGGPSV |
| | | FLFPPKPKDTLMISRTPEVTCVVVDVSHEDPEVKFNWYVDGVEVHNAKT |
| | | KPREEQYASTYRVVSVLTVLHQDWLNGKEYKCKVSNKALPAPIEKTISKA |
| | | KGQPREPQVYTLPPCREEMTKNQVSLWCLVKGFYPSDIAVEWESNGQPE |
| | | NNYKTTPPVLDSDGSFFLYSKLTVDKSRWQQGNVFSCSVMHEALHNHYT |
| | | QKSLSLSPGK |
| SEQ | anti-TRBC1 | QVQLVQSGAEVKKPGSSVKVSCKASGYTFTGYVMHWVRQAPGQGLEW |
| ID | HC | MGFIIPIFGTANYAQKFQGRVTITSDKSTTTAYMELSSLRSEDTAVYYCA |
| NO: | | RGAGYNFDGAYRFFDFWGQGTLVTVSSASTKGPSVFPLAPSSKSTSGGT |
| 4677 | | AALGCLVKDYFPEPVTVSWNSGALTSGVHTFPAVLQSSGLYSLSSVVTVP |
| | | SSSLGTQTYICNVNHKPSNTKVDKRVEPKSCDKTHTCPPCPAPELLGGPSV |
| | | FLFPPKPKDTLMISRTPEVTCVVVDVSHEDPEVKFNWYVDGVEVHNAKT |
| | | KPREEQYASTYRVVSVLTVLHQDWLNGKEYKCKVSNKALPAPIEKTISKA |
| | | KGQPREPQVYTLPPCREEMTKNQVSLWCLVKGFYPSDIAVEWESNGQPE |
| | | NNYKTTPPVLDSDGSFFLYSKLTVDKSRWQQGNVFSCSVMHEALHNHYT |
| | | QKSLSLSP |
| SEQ | anti-TRBC1 | DVVMTQSPLSLPVTLGQPASISCRSSQRLVHSNGNTYLHWYQQRPGQSP |
| ID | LC | RLLIYRVSNRFPGVPDRFSGSGSGTDFTLKISRVEAEDVGVYFCSQSTHVP |
| NO: | | YTFGGGTKVEIKRTVAAPSVFIFPPSDEQLKSGTASVVCLLNNFYPREAKV |
| 7380 | | QWKVDNALQSGNSQESVTEQDSKDSTYSLSSTLTLSKADYEKHKVYACE |
| | | VTHQGLSSPVTKSFNRGEC |
| SEQ | anti-NKp30 | EIQLLESGGGLVQPGGSLRLSCAVSGFSITTTGYHWNWVRQAPGKGLEW |
| ID | humanized | VGYIYSSGSTSYNPSLKSRFTISRDTSKNTFYLQMNSLRAEDTAVYYCARG |
| NO: | 15E1 scFv- | DWHYFDYWGQGTMVTVSSGGGGSGGGGSGGGGSGGGGSDSVTTQSPLS |
| 7383 | Fc | LPVTLGQPASISCSGEKLSDKYVHWYQQRPGQSPRMLIYENDRRPSGVPD |
| | | RFSGSNSGNDATLKISRVEAEDVGVYFCQFWDSTNSAVFGGGTKVEIKDK |
| | | THTCPPCPAPELLGGPSVFLFPPKPKDTLMISRTPEVTCVVVDVSHEDPEV |
| | | KFNWYVDGVEVHNAKTKPREEQYASTYRVVSVLTVLHQDWLNGKEYK |
| | | CKVSNKALPAPIEKTISKAKGQPREPQVCTLPPSREEMTKNQVSLSCAVKG |
| | | FYPSDIAVEWESNGQPENNYKTTPPVLDSDGSFFLVSKLTVDKSRWQQGN |
| | | VFSCSVMHEALHNHYTQKSLSLSPGK |
| SEQ | anti-NKp30 | EIQLLESGGGLVQPGGSLRLSCAVSGFSITTTGYHWNWVRQAPGKGLEW |
| ID | humanized | VGYIYSSGSTSYNPSLKSRFTISRDTSKNTFYLQMNSLRAEDTAVYYCARG |
| NO: | 15E1 scFv- | DWHYFDYWGQGTMVTVSSGGGGSGGGGSGGGGSGGGGSDSVTTQSPLS |
| 4678 | Fc | LPVTLGQPASISCSGEKLSDKYVHWYQQRPGQSPRMLIYENDRRPSGVPD |
| | | RFSGSNSGNDATLKISRVEAEDVGVYFCQFWDSTNSAVFGGGTKVEIKDK |
| | | THTCPPCPAPELLGGPSVFLFPPKPKDTLMISRTPEVTCVVVDVSHEDPEV |
| | | KFNWYVDGVEVHNAKTKPREEQYASTYRVVSVLTVLHQDWLNGKEYK |
| | | CKVSNKALPAPIEKTISKAKGQPREPQVCTLPPSREEMTKNQVSLSCAVKG |
| | | FYPSDIAVEWESNGQPENNYKTTPPVLDSDGSFFLVSKLTVDKSRWQQGN |
| | | VFSCSVMHEALHNHYTQKSLSLSP |

Anti-TRBC1-NKp30-BJM1052

| | | |
|---|---|---|
| SEQ | anti-TRBC1 | QVQLVQSGAEVKKPGSSVKVSCKASGYTFTGYVMHWVRQAPGQGLEW |
| ID | HC | MGFINPYNDDIQSNERFRGRVTITSDKSTTTAYMELSSLRSEDTAVYYCA |
| NO: | | RGAGYNFDGAYRFFDFWGQGTLVTVSSASTKGPSVFPLAPSSKSTSGGT |
| 7379 | | AALGCLVKDYFPEPVTVSWNSGALTSGVHTFPAVLQSSGLYSLSSVVTVP |
| | | SSSLGTQTYICNVNHKPSNTKVDKRVEPKSCDKTHTCPPCPAPELLGGPSV |
| | | FLFPPKPKDTLMISRTPEVTCVVVDVSHEDPEVKFNWYVDGVEVHNAKT |
| | | KPREEQYASTYRVVSVLTVLHQDWLNGKEYKCKVSNKALPAPIEKTISKA |
| | | KGQPREPQVYTLPPCREEMTKNQVSLWCLVKGFYPSDIAVEWESNGQPE |
| | | NNYKTTPPVLDSDGSFFLYSKLTVDKSRWQQGNVFSCSVMHEALHNHYT |
| | | QKSLSLSPGK |
| SEQ | anti-TRBC1 | QVQLVQSGAEVKKPGSSVKVSCKASGYTFTGYVMHWVRQAPGQGLEW |
| ID | HC | MGFINPYNDDIQSNERFRGRVTITSDKSTTTAYMELSSLRSEDTAVYYCA |
| NO: | | RGAGYNFDGAYRFFDFWGQGTLVTVSSASTKGPSVFPLAPSSKSTSGGT |
| 4675 | | AALGCLVKDYFPEPVTVSWNSGALTSGVHTFPAVLQSSGLYSLSSVVTVP |
| | | SSSLGTQTYICNVNHKPSNTKVDKRVEPKSCDKTHTCPPCPAPELLGGPSV |
| | | FLFPPKPKDTLMISRTPEVTCVVVDVSHEDPEVKFNWYVDGVEVHNAKT |
| | | KPREEQYASTYRVVSVLTVLHQDWLNGKEYKCKVSNKALPAPIEKTISKA |
| | | KGQPREPQVYTLPPCREEMTKNQVSLWCLVKGFYPSDIAVEWESNGQPE |
| | | NNYKTTPPVLDSDGSFFLYSKLTVDKSRWQQGNVFSCSVMHEALHNHYT |
| | | QKSLSLSP |
| SEQ | anti-TRBC1 | DVVMTQSPLSLPVTLGQPASISCRSSQRLVHSNGNTYLHWYQQRPGQSP |
| ID | LC | RLLIYRVSNRFPGVPDRFSGSGSGTDFTLKISRVEAEDVGVYFCSQSTHVP |
| NO: | | YTFGGGTKVEIKRTVAAPSVFIFPPSDEQLKSGTASVVCLLNNFYPREAKV |
| 7380 | | QWKVDNALQSGNSQESVTEQDSKDSTYSLSSTLTLSKADYEKHKVYACE |
| | | VTHQGLSSPVTKSFNRGEC |
| SEQ | anti-NKp30 | EIQLLESGGGLVQPGGSLRLSCAVSGFSITTTGYHWNWVRQAPGKGLEW |
| ID | humanized | VGYIYSSGSTSYNPSLKSRFTISRDTSKNTFYLQMNSLRAEDTAVYYCARG |
| NO | 15E1 scFv- | DWHYFDYWGQGTMVTVSSGGGGSGGGGSGGGGSGGGGSDSVTTQSPLS |
| 7383 | Fc | LPVTLGQPASISCSGEKLSDKYVHWYQQRPGQSPRMLIYENDRRPSGVPD |
| | | RFSGSNSGNDATLKISRVEAEDVGVYFCQFWDSTNSAVFGGGTKVEIKDK |
| | | THTCPPCPAPELLGGPSVFLFPPKPKDTLMISRTPEVTCVVVDVSHEDPEV |
| | | KFNWYVDGVEVHNAKTKPREEQYASTYRVVSVLTVLHQDWLNGKEYK |
| | | CKVSNKALPAPIEKTISKAKGQPREPQVCTLPPSREEMTKNQVSLSCAVKG |
| | | FYPSDIAVEWESNGQPENNYKTTPPVLDSDGSFFLVSKLTVDKSRWQQGN |
| | | VFSCSVMHEALHNHYTQKSLSLSPGK |
| SEQ | anti-NKp30 | EIQLLESGGGLVQPGGSLRLSCAVSGFSITTTGYHWNWVRQAPGKGLEW |
| ID | humanized | VGYIYSSGSTSYNPSLKSRFTISRDTSKNTFYLQMNSLRAEDTAVYYCARG |
| NO: | 15E1 scFv- | DWHYFDYWGQGTMVTVSSGGGGSGGGGSGGGGSGGGGSDSVTTQSPLS |
| 4678 | Fc | LPVTLGQPASISCSGEKLSDKYVHWYQQRPGQSPRMLIYENDRRPSGVPD |
| | | RFSGSNSGNDATLKISRVEAEDVGVYFCQFWDSTNSAVFGGGTKVEIKDK |
| | | THTCPPCPAPELLGGPSVFLFPPKPKDTLMISRTPEVTCVVVDVSHEDPEV |
| | | KFNWYVDGVEVHNAKTKPREEQYASTYRVVSVLTVLHQDWLNGKEYK |
| | | CKVSNKALPAPIEKTISKAKGQPREPQVCTLPPSREEMTKNQVSLSCAVKG |
| | | FYPSDIAVEWESNGQPENNYKTTPPVLDSDGSFFLVSKLTVDKSRWQQGN |
| | | VFSCSVMHEALHNHYTQKSLSLSP |

Anti-TRBC1-NKp30-BJM1038

| | | |
|---|---|---|
| SEQ | anti-TRBC1 | QVQLVQSGAEVKKPGSSVKVSCKASGYTFTGYVMHWVRQAPGQGLEW |
| ID | HC | MGFIIPIFGTANYAQKFQGRVTITSDKSTTTAYMELSSLRSEDTAVYYCA |
| NO: | | RGAGYNFDGAYRFFDFWGQGTLVTVSSASTKGPSVFPLAPSSKSTSGGT |
| 7382 | | AALGCLVKDYFPEPVTVSWNSGALTSGVHTFPAVLQSSGLYSLSSVVTVP |
| | | SSSLGTQTYICNVNHKPSNTKVDKRVEPKSCDKTHTCPPCPAPELLGGPSV |
| | | FLFPPKPKDTLMISRTPEVTCVVVDVSHEDPEVKFNWYVDGVEVHNAKT |
| | | KPREEQYASTYRVVSVLTVLHQDWLNGKEYKCKVSNKALPAPIEKTISKA |
| | | KGQPREPQVYTLPPCREEMTKNQVSLWCLVKGFYPSDIAVEWESNGQPE |
| | | NNYKTTPPVLDSDGSFFLYSKLTVDKSRWQQGNVFSCSVMHEALHNHYT |
| | | QKSLSLSPGK |
| SEQ | anti-TRBC1 | QVQLVQSGAEVKKPGSSVKVSCKASGYTFTGYVMHWVRQAPGQGLEW |
| ID | HC | MGFIIPIFGTANYAQKFQGRVTITSDKSTTTAYMELSSLRSEDTAVYYCA |
| NO: | | RGAGYNFDGAYRFFDFWGQGTLVTVSSASTKGPSVFPLAPSSKSTSGGT |
| 4677 | | AALGCLVKDYFPEPVTVSWNSGALTSGVHTFPAVLQSSGLYSLSSVVTVP |
| | | SSSLGTQTYICNVNHKPSNTKVDKRVEPKSCDKTHTCPPCPAPELLGGPSV |
| | | FLFPPKPKDTLMISRTPEVTCVVVDVSHEDPEVKFNWYVDGVEVHNAKT |
| | | KPREEQYASTYRVVSVLTVLHQDWLNGKEYKCKVSNKALPAPIEKTISKA |
| | | KGQPREPQVYTLPPCREEMTKNQVSLWCLVKGFYPSDIAVEWESNGQPE |
| | | NNYKTTPPVLDSDGSFFLYSKLTVDKSRWQQGNVFSCSVMHEALHNHYT |
| | | QKSLSLSP |
| SEQ | anti-TRBC1 | DVVMTQSPLSLPVTLGQPASISCRSSQRLVHSNGNTYLHWYQQRPGQSP |
| ID | LC | RLLIYRVSNRFPGVPDRFSGSGSGTDFTLKISRVEAEDVGVYFCSQSTHVP |
| NO: | | YTFGGGTKVEIKRTVAAPSVFIFPPSDEQLKSGTASVVCLLNNFYPREAKV |
| 7380 | | QWKVDNALQSGNSQESVTEQDSKDSTYSLSSTLTLSKADYEKHKVYACE |
| | | VTHQGLSSPVTKSFNRGEC |
| SEQ | anti-NKp30 | EIQLLESGGGLVQPGGSLRLSCAVSGFSITTTGYHWNWVRQAPGKGLEW |
| ID | humanized | VGYIYSSGSTSYNPSLKSRFTISRDTSKNTFYLQMNSLRAEDTAVYYCARG |
| NO: | 15E1 scFv- | DWHYFDYWGQGTMVTVSSGGGGSGGGGSGGGGSGGGGSSSETTQPPSV |
| 7384 | Fc | SVSPGQTASITCSGEKLSDKYVHWYQQKPGQSPVMVIYENDRRPSGIPERF |
| | | SGSNSGNTATLTISGTQAMDEADYFCQFWDSTNSAVFGGGTQLTVLDKT |
| | | HTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCK |
| | | VSNKALPAPIEKTISKAKGQPREPQVCTLPPSREEMTKNQVSLSCAVKGFY |
| | | PSDIAVEWESNGQPENNYKTTPPVLDSDGSFFLVSKLTVDKSRWQQGNVF |
| | | SCSVMHEALHNHYTQKSLSLSPGK |
| SEQ | anti-NKp30 | EIQLLESGGGLVQPGGSLRLSCAVSGFSITTTGYHWNWVRQAPGKGLEW |
| ID | humanized | VGYIYSSGSTSYNPSLKSRFTISRDTSKNTFYLQMNSLRAEDTAVYYCARG |
| NO: | 15E1 scFv- | DWHYFDYWGQGTMVTVSSGGGGSGGGGSGGGGSGGGGSSSETTQPPSV |
| 4679 | Fc | SVSPGQTASITCSGEKLSDKYVHWYQQKPGQSPVMVIYENDRRPSGIPERF |
| | | SGSNSGNTATLTISGTQAMDEADYFCQFWDSTNSAVFGGGTQLTVLDKT |
| | | HTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCK |
| | | VSNKALPAPIEKTISKAKGQPREPQVCTLPPSREEMTKNQVSLSCAVKGFY |
| | | PSDIAVEWESNGQPENNYKTTPPVLDSDGSFFLVSKLTVDKSRWQQGNVF |
| | | SCSVMHEALHNHYTQKSLSLSP |

Anti-TRBC1-NKp30-BJM1048

| | | |
|---|---|---|
| SEQ | anti-TRBC1 | QVQLVQSGAEVKKPGSSVKVSCKASGYTFTGYVMHWVRQAPGQGLEW |
| ID | HC | MGFINPYNDDIQSNERFRGRVTITSDKSTTTAYMELSSLRSEDTAVYYCA |
| NO: | | RGAGYNFDGAYRFFDFWGQGTLVTVSSASTKGPSVFPLAPSSKSTSGGT |
| 7379 | | AALGCLVKDYFPEPVTVSWNSGALTSGVHTFPAVLQSSGLYSLSSVVTVP |
| | | SSSLGTQTYICNVNHKPSNTKVDKRVEPKSCDKTHTCPPCPAPELLGGPSV |
| | | FLFPPKPKDTLMISRTPEVTCVVVDVSHEDPEVKFNWYVDGVEVHNAKT |
| | | KPREEQYASTYRVVSVLTVLHQDWLNGKEYKCKVSNKALPAPIEKTISKA |
| | | KGQPREPQVYTLPPCREEMTKNQVSLWCLVKGFYPSDIAVEWESNGQPE |
| | | NNYKTTPPVLDSDGSFFLYSKLTVDKSRWQQGNVFSCSVMHEALHNHYT |
| | | QKSLSLSPGK |
| SEQ | anti-TRBC1 | QVQLVQSGAEVKKPGSSVKVSCKASGYTFTGYVMHWVRQAPGQGLEW |
| ID | HC | MGFINPYNDDIQSNERFRGRVTITSDKSTTTAYMELSSLRSEDTAVYYCA |
| NO: | | RGAGYNFDGAYRFFDFWGQGTLVTVSSASTKGPSVFPLAPSSKSTSGGT |
| 4675 | | AALGCLVKDYFPEPVTVSWNSGALTSGVHTFPAVLQSSGLYSLSSVVTVP |
| | | SSSLGTQTYICNVNHKPSNTKVDKRVEPKSCDKTHTCPPCPAPELLGGPSV |
| | | FLFPPKPKDTLMISRTPEVTCVVVDVSHEDPEVKFNWYVDGVEVHNAKT |
| | | KPREEQYASTYRVVSVLTVLHQDWLNGKEYKCKVSNKALPAPIEKTISKA |
| | | KGQPREPQVYTLPPCREEMTKNQVSLWCLVKGFYPSDIAVEWESNGQPE |
| | | NNYKTTPPVLDSDGSFFLYSKLTVDKSRWQQGNVFSCSVMHEALHNHYT |
| | | QKSLSLSP |
| SEQ | anti-TRBC1 | DVVMTQSPLSLPVTLGQPASISCRSSQRLVHSNGNTYLHWYQQRPGQSP |
| ID | LC | RLLIYRVSNRFPGVPDRFSGSGSGTDFTLKISRVEAEDVGVYFCSQSTHVP |
| NO: | | YTFGGGTKVEIKRTVAAPSVFIFPPSDEQLKSGTASVVCLLNNFYPREAKV |
| 7380 | | QWKVDNALQSGNSQESVTEQDSKDSTYSLSSTLTLSKADYEKHKVYACE |
| | | VTHQGLSSPVTKSFNRGEC |
| SEQ | anti-NKp30 | EIQLLESGGGLVQPGGSLRLSCAVSGFSITTTGYHWNWVRQAPGKGLEW |
| ID | humanized | VGYIYSSGSTSYNPSLKSRFTISRDTSKNTFYLQMNSLRAEDTAVYYCARG |
| NO: | 15E1 scFv- | DWHYFDYWGQGTMVTVSSGGGGSGGGGSGGGGSGGGGSSSETTQPPSV |
| 7384 | Fc | SVSPGQTASITCSGEKLSDKYVHWYQQKPGQSPVMVIYENDRRPSGIPERF |
| | | SGSNSGNTATLTISGTQAMDEADYFCQFWDSTNSAVFGGGTQLTVLDKT |
| | | HTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCK |
| | | VSNKALPAPIEKTISKAKGQPREPQVCTLPPSREEMTKNQVSLSCAVKGFY |
| | | PSDIAVEWESNGQPENNYKTTPPVLDSDGSFFLVSKLTVDKSRWQQGNVF |
| | | SCSVMHEALHNHYTQKSLSLSPGK |
| SEQ | anti-NKp30 | EIQLLESGGGLVQPGGSLRLSCAVSGFSITTTGYHWNWVRQAPGKGLEW |
| ID | humanized | VGYIYSSGSTSYNPSLKSRFTISRDTSKNTFYLQMNSLRAEDTAVYYCARG |
| NO: | 15E1 scFv- | DWHYFDYWGQGTMVTVSSGGGGSGGGGSGGGGSGGGGSSSETTQPPSV |
| 4679 | Fc | SVSPGQTASITCSGEKLSDKYVHWYQQKPGQSPVMVIYENDRRPSGIPERF |
| | | SGSNSGNTATLTISGTQAMDEADYFCQFWDSTNSAVFGGGTQLTVLDKT |
| | | HTCPPCPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYKCK |
| | | VSNKALPAPIEKTISKAKGQPREPQVCTLPPSREEMTKNQVSLSCAVKGFY |
| | | PSDIAVEWESNGQPENNYKTTPPVLDSDGSFFLVSKLTVDKSRWQQGNVF |
| | | SCSVMHEALHNHYTQKSLSLSP |

TABLE 17
Exemplary multispecific antibody molecules or parts thereof that bind to TRBC2
AND/OR NKp30

| | | |
|---|---|---|
| SEQ | | |
| ID NO | Description | Sequence |

Anti-TRBC2-NKp30-BKM0097

| | | |
|---|---|---|
| SEQ | Anti- | QVQLVQSGAEVKKPGASVKVSCKASTSGFHGYPMHWVRQAPGQGLEWMGFI |
| ID | TRBC | NPYNDDIQSNERFRGRVTMTSDKSTTTAYMELSSLRSEDTAVYYCARGNGKW |
| NO: | 2 HC | GDGAYRFFDFWGQGTLVTVSSASTKGPSVFPLAPSSKSTSGGTAALGCLVKDY |
| 7438 | | FPEPVTVSWNSGALTSGVHTFPAVLQSSGLYSLSSVVTVPSSSLGTQTYICNVNH |
| | | KPSNTKVDKRVEPKSCDKTHTCPPCPAPELLGGPSVFLFPPKPKDTLMISRTPEV |
| | | TCVVVDVSHEDPEVKFNWYVDGVEVHNAKTKPREEQYASTYRVVSVLTVLH |
| | | QDWLNGKEYKCKVSNKALPAPIEKTISKAKGQPREPQVYTLPPCREEMTKNQV |
| | | SLWCLVKGFYPSDIAVEWESNGQPENNYKTTPPVLDSDGSFFLYSKLTVDKSR |
| | | WQQGNVFSCSVMHEALHNHYTQKSLSLSPGK |
| SEQ | Anti- | QVQLVQSGAEVKKPGASVKVSCKASTSGFHGYPMHWVRQAPGQGLEWMGFI |
| ID | TRBC | NPYNDDIQSNERFRGRVTMTSDKSTTTAYMELSSLRSEDTAVYYCARGNGKW |
| NO: | 2 HC | GDGAYRFFDFWGQGTLVTVSSASTKGPSVFPLAPSSKSTSGGTAALGCLVKDY |
| 4680 | | FPEPVTVSWNSGALTSGVHTFPAVLQSSGLYSLSSVVTVPSSSLGTQTYICNVNH |
| | | KPSNTKVDKRVEPKSCDKTHTCPPCPAPELLGGPSVFLFPPKPKDTLMISRTPEV |
| | | TCVVVDVSHEDPEVKFNWYVDGVEVHNAKTKPREEQYASTYRVVSVLTVLH |
| | | QDWLNGKEYKCKVSNKALPAPIEKTISKAKGQPREPQVYTLPPCREEMTKNQV |
| | | SLWCLVKGFYPSDIAVEWESNGQPENNYKTTPPVLDSDGSFFLYSKLTVDKSR |
| | | WQQGNVFSCSVMHEALHNHYTQKSLSLSP |
| SEQ | Anti- | DVVMTQSPLSLPVTPGEPASISCRSSQNLVHSNGRTYLQWYLQKPGQSPQLLIY |
| ID | TRBC | RVSNRFPGVPDRFSGSGSGTDFTLKISRVEAEDVGVYFCSQSTHVPYTFGGGTK |
| NO: | 2 LC | VEIKRTVAAPSVFIFPPSDEQLKSGTASVVCLLNNFYPREAKVQWKVDNALQSG |
| 7439 | | NSQESVTEQDSKDSTYSLSSTLTLSKADYEKHKVYACEVTHQGLSSPVTKSFNR |
| | | GEC |
| SEQ | anti- | EIQLLESGGGLVQPGGSLRLSCAVSGFSITTTGYHWNWVRQAPGKGLEWVGYI |
| ID | NKp30 | YSSGSTSYNPSLKSRFTISRDTSKNTFYLQMNSLRAEDTAVYYCARGDWHYFD |
| NO: | human | YWGQGTMVTVSSGGGGSGGGGSGGGGSGGGGSDSVTTQSPLSLPVTLGQPASI |
| 7383 | ized | SCSGEKLSDKYVHWYQQRPGQSPRMLIYENDRRPSGVPDRFSGSNSGNDATLK |
| | 15E1 | ISRVEAEDVGVYFCQFWDSTNSAVFGGGTKVEIKDKTHTCPPCPAPELLGGPSV |
| | scFv- | FLFPPKPKDTLMISRTPEVTCVVVDVSHEDPEVKFNWYVDGVEVHNAKTKPRE |
| | Fc | EQYASTYRVVSVLTVLHQDWLNGKEYKCKVSNKALPAPIEKTISKAKGQPREP |
| | | QVCTLPPSREEMTKNQVSLSCAVKGFYPSDIAVEWESNGQPENNYKTTPPVLDS |
| | | DGSFFLVSKLTVDKSRWQQGNVFSCSVMHEALHNHYTQKSLSLSPGK |
| SEQ | anti- | EIQLLESGGGLVQPGGSLRLSCAVSGFSITTTGYHWNWVRQAPGKGLEWVGYI |
| ID | NKp30 | YSSGSTSYNPSLKSRFTISRDTSKNTFYLQMNSLRAEDTAVYYCARGDWHYFD |
| NO: | human | YWGQGTMVTVSSGGGGSGGGGSGGGGSGGGGSDSVTTQSPLSLPVTLGQPASI |
| 4678 | ized | SCSGEKLSDKYVHWYQQRPGQSPRMLIYENDRRPSGVPDRFSGSNSGNDATLK |
| | 15E1 | ISRVEAEDVGVYFCQFWDSTNSAVFGGGTKVEIKDKTHTCPPCPAPELLGGPSV |
| | scFv- | FLFPPKPKDTLMISRTPEVTCVVVDVSHEDPEVKFNWYVDGVEVHNAKTKPRE |
| | Fc | EQYASTYRVVSVLTVLHQDWLNGKEYKCKVSNKALPAPIEKTISKAKGQPREP |
| | | QVCTLPPSREEMTKNQVSLSCAVKGFYPSDIAVEWESNGQPENNYKTTPPVLDS |
| | | DGSFFLVSKLTVDKSRWQQGNVFSCSVMHEALHNHYTQKSLSLSP |

Anti-TRBC2-NKp30-BKM0098

| | | |
|---|---|---|
| SEQ | Anti- | QVQLVQSGAEVKKPGASVKVSCKASPRGFHGYHMHWVRQAPGQGLEWMGFI |
| ID | TRBC | NPYNDDIQSNERFRGRVTMTSDKSTTTAYMELSSLRSEDTAVYYCARGNGKW |
| NO: | 2 HC | GDGAYRFFDFWGQGTLVTVSSASTKGPSVFPLAPSSKSTSGGTAALGCLVKDY |
| 7440 | | FPEPVTVSWNSGALTSGVHTFPAVLQSSGLYSLSSVVTVPSSSLGTQTYICNVNH |
| | | KPSNTKVDKRVEPKSCDKTHTCPPCPAPELLGGPSVFLFPPKPKDTLMISRTPEV |
| | | TCVVVDVSHEDPEVKFNWYVDGVEVHNAKTKPREEQYASTYRVVSVLTVLH |
| | | QDWLNGKEYKCKVSNKALPAPIEKTISKAKGQPREPQVYTLPPCREEMTKNQV |
| | | SLWCLVKGFYPSDIAVEWESNGQPENNYKTTPPVLDSDGSFFLYSKLTVDKSR |
| | | WQQGNVFSCSVMHEALHNHYTQKSLSLSPGK |
| SEQ | Anti- | QVQLVQSGAEVKKPGASVKVSCKASPRGFHGYHMHWVRQAPGQGLEWMGFI |
| ID | TRBC | NPYNDDIQSNERFRGRVTMTSDKSTTTAYMELSSLRSEDTAVYYCARGNGKW |
| NO: | 2 HC | GDGAYRFFDFWGQGTLVTVSSASTKGPSVFPLAPSSKSTSGGTAALGCLVKDY |
| 4681 | | FPEPVTVSWNSGALTSGVHTFPAVLQSSGLYSLSSVVTVPSSSLGTQTYICNVNH |
| | | KPSNTKVDKRVEPKSCDKTHTCPPCPAPELLGGPSVFLFPPKPKDTLMISRTPEV |
| | | TCVVVDVSHEDPEVKFNWYVDGVEVHNAKTKPREEQYASTYRVVSVLTVLH |
| | | QDWLNGKEYKCKVSNKALPAPIEKTISKAKGQPREPQVYTLPPCREEMTKNQV |
| | | SLWCLVKGFYPSDIAVEWESNGQPENNYKTTPPVLDSDGSFFLYSKLTVDKSR |
| | | WQQGNVFSCSVMHEALHNHYTQKSLSLSP |
| SEQ | Anti- | DVVMTQSPLSLPVTPGEPASISCRSSQNLVHSNGRTYLQWYLQKPGQSPQLLIY |
| ID | TRBC | RVSNRFPGVPDRFSGSGSGTDFTLKISRVEAEDVGVYFCSQSTHVPYTFGGGTK |
| NO: | 2 LC | VEIKRTVAAPSVFIFPPSDEQLKSGTASVVCLLNNFYPREAKVQWKVDNALQSG |
| 7439 | | NSQESVTEQDSKDSTYSLSSTLTLSKADYEKHKVYACEVTHQGLSSPVTKSFNR |
| | | GEC |
| SEQ | anti- | EIQLLESGGGLVQPGGSLRLSCAVSGFSITTTGYHWNWVRQAPGKGLEWVGYI |
| ID | NKp30 | YSSGSTSYNPSLKSRFTISRDTSKNTFYLQMNSLRAEDTAVYYCARGDWHYFD |
| NO: | human | YWGQGTMVTVSSGGGGSGGGGSGGGGSGGGGSDSVTTQSPLSLPVTLGQPASI |
| 7383 | ized | SCSGEKLSDKYVHWYQQRPGQSPRMLIYENDRRPSGVPDRFSGSNSGNDATLK |
| | 15E1 | ISRVEAEDVGVYFCQFWDSTNSAVFGGGTKVEIKDKTHTCPPCPAPELLGGPSV |
| | scFv- | FLFPPKPKDTLMISRTPEVTCVVVDVSHEDPEVKFNWYVDGVEVHNAKTKPRE |
| | Fc | EQYASTYRVVSVLTVLHQDWLNGKEYKCKVSNKALPAPIEKTISKAKGQPREP |
| | | QVCTLPPSREEMTKNQVSLSCAVKGFYPSDIAVEWESNGQPENNYKTTPPVLDS |
| | | DGSFFLVSKLTVDKSRWQQGNVFSCSVMHEALHNHYTQKSLSLSPGK |
| SEQ | anti- | EIQLLESGGGLVQPGGSLRLSCAVSGFSITTTGYHWNWVRQAPGKGLEWVGYI |
| ID | NKp30 | YSSGSTSYNPSLKSRFTISRDTSKNTFYLQMNSLRAEDTAVYYCARGDWHYFD |
| NO: | human | YWGQGTMVTVSSGGGGSGGGGSGGGGSGGGGSDSVTTQSPLSLPVTLGQPASI |
| 4678 | ized | SCSGEKLSDKYVHWYQQRPGQSPRMLIYENDRRPSGVPDRFSGSNSGNDATLK |
| | 15E1 | ISRVEAEDVGVYFCQFWDSTNSAVFGGGTKVEIKDKTHTCPPCPAPELLGGPSV |
| | scFv- | FLFPPKPKDTLMISRTPEVTCVVVDVSHEDPEVKFNWYVDGVEVHNAKTKPRE |
| | Fc | EQYASTYRVVSVLTVLHQDWLNGKEYKCKVSNKALPAPIEKTISKAKGQPREP |
| | | QVCTLPPSREEMTKNQVSLSCAVKGFYPSDIAVEWESNGQPENNYKTTPPVLDS |
| | | DGSFFLVSKLTVDKSRWQQGNVFSCSVMHEALHNHYTQKSLSLSP |

anti-TRBC2-NKp30 - BIS-2020-1

| | | |
|---|---|---|
| SEQ | Anti- | QVQLVQSGAEVKKPGSSVKVSCKASPRGFYGYHMHWVRQAPGQGLEWMGFI |
| ID | TRBC | NPYTNDIQYNERFRGRVTITSDESTTTAYMELSSLRSEDTAVYYCAMGNGKWG |
| NO: | 2 - | DGAYRFFDLWGQGTLVTVSSASTKGPSVFPLAPSSKSTSGGTAALGCLVKDYFP |
| 8001 | BKM0238- | EPVTVSWNSGALTSGVHTFPAVLQSSGLYSLSSVVTVPSSSLGTQTYICNVNHK |
| | HC 1 | PSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYASTYRVVSVLTVLHQ |
| | | DWLNGKEYKCKVSNKALPAPIEKTISKAKGQPREPQVYTLPPCREEMTKNQVS |
| | | LWCLVKGFYPSDIAVEWESNGQPENNYKTTPPVLDSDGSFFLYSKLTVDKSRW |
| | | QQGNVFSCSVMHEALHNHYTQKSLSLSPGK |
| SEQ | Anti- | QVQLVQSGAEVKKPGSSVKVSCKASPRGFYGYHMHWVRQAPGQGLEWMGFI |
| ID | TRBC | NPYTNDIQYNERFRGRVTITSDESTTTAYMELSSLRSEDTAVYYCAMGNGKWG |
| NO: | 2 - | DGAYRFFDLWGQGTLVTVSSASTKGPSVFPLAPSSKSTSGGTAALGCLVKDYFP |
| 4682 | BKM0238- | EPVTVSWNSGALTSGVHTFPAVLQSSGLYSLSSVVTVPSSSLGTQTYICNVNHK |
| | HC 1 | PSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYASTYRVVSVLTVLHQ |
| | | DWLNGKEYKCKVSNKALPAPIEKTISKAKGQPREPQVYTLPPCREEMTKNQVS |
| | | LWCLVKGFYPSDIAVEWESNGQPENNYKTTPPVLDSDGSFFLYSKLTVDKSRW |
| | | QQGNVFSCSVMHEALHNHYTQKSLSLSP |
| SEQ | Anti- | DVVMTQSPLSLPVTLGQPASISCRSSENLVHSNGRTYLQWYQQRPGQSPRLLIY |
| ID | TRBC | RVSNRFPGVPDRFSGSGSGTDFTLKISRVEAEDVGVYFCSQSSLEPYTFGGGTK |
| NO: | 2- | VEIKRTVAAPSVFIFPPSDEQLKSGTASVVCLLNNFYPREAKVQWKVDNALQSG |
| 8002 | BKM0238- | NSQESVTEQDSKDSTYSLSSTLTLSKADYEKHKVYACEVTHQGLSSPVTKSFNR |
| | LC | GEC |
| SEQ | Nk- | EIQLLESGGGLVQPGGSLRLSCAVSGFSITTTGYHWNWVRQAPGKGLEWVGYI |
| ID | p30 | YSSGSTSYNPSLKSRFTISRDTSKNTFYLQMNSLRAEDTAVYYCARGDWHYFD |
| NO: | scFv | YWGQGTMVTVSSGGGGSGGGGSGGGGSGGGGSDSVTTQSPLSLPVTLGQPASI |
| 8693/ | | SCSGEKLSDKYVHWYQQRPGQSPRMLIYENDRRPSGVPDRFSGSNSGNDATLK |
| SEQ | | ISRVEAEDVGVYFCQFWDSTNSAVFGGGTKVEIKDKTHTCPPCPAPELLGGPSV |
| ID | | FLFPPKPKDTLMISRTPEVTCVVVDVSHEDPEVKFNWYVDGVEVHNAKTKPRE |
| NO: | | EQYASTYRVVSVLTVLHQDWLNGKEYKCKVSNKALPAPIEKTISKAKGQPREP |
| 8003 | | QVCTLPPSREEMTKNQVSLSCAVKGFYPSDIAVEWESNGQPENNYKTTPPVLDS |
| | | DGSFFLVSKLTVDKSRWQQGNVFSCSVMHEALHNHYTQKSLSLSPGK |
| SEQ | Nk- | EIQLLESGGGLVQPGGSLRLSCAVSGFSITTTGYHWNWVRQAPGKGLEWVGYI |
| ID | p30 | YSSGSTSYNPSLKSRFTISRDTSKNTFYLQMNSLRAEDTAVYYCARGDWHYFD |
| NO: | scFv | YWGQGTMVTVSSGGGGSGGGGSGGGGSGGGGSDSVTTQSPLSLPVTLGQPASI |
| 4683 | | SCSGEKLSDKYVHWYQQRPGQSPRMLIYENDRRPSGVPDRFSGSNSGNDATLK |
| | | ISRVEAEDVGVYFCQFWDSTNSAVFGGGTKVEIKDKTHTCPPCPAPELLGGPSV |
| | | FLFPPKPKDTLMISRTPEVTCVVVDVSHEDPEVKFNWYVDGVEVHNAKTKPRE |
| | | EQYASTYRVVSVLTVLHQDWLNGKEYKCKVSNKALPAPIEKTISKAKGQPREP |
| | | QVCTLPPSREEMTKNQVSLSCAVKGFYPSDIAVEWESNGQPENNYKTTPPVLDS |
| | | DGSFFLVSKLTVDKSRWQQGNVFSCSVMHEALHNHYTQKSLSLSP |

anti-TRBC2-NKp30 - BIS-2020-2

| | | |
|---|---|---|
| SEQ | Anti- | QVQLVQSGAEVKKPGSSVKVSCKASPRGFYGYHMHWVRQAPGQGLEWMGFI |
| ID | TRBC | NPYTNDIQYNERFRGRVTITSDESTTTAYMELSSLRSEDTAVYYCAMGNGKWG |
| NO: | 2- | DGAYRFFDLWGQGTLVTVSSASTKGPSVFPLAPSSKSTSGGTAALGCLVKDYFP |
| 8001 | BKM0238- | EPVTVSWNSGALTSGVHTFPAVLQSSGLYSLSSVVTVPSSSLGTQTYICNVNHK |
| | HC 1 | PSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYASTYRVVSVLTVLHQ |
| | | DWLNGKEYKCKVSNKALPAPIEKTISKAKGQPREPQVYTLPPCREEMTKNQVS |
| | | LWCLVKGFYPSDIAVEWESNGQPENNYKTTPPVLDSDGSFFLYSKLTVDKSRW |
| | | QQGNVFSCSVMHEALHNHYTQKSLSLSPGK |
| SEQ | Anti- | QVQLVQSGAEVKKPGSSVKVSCKASPRGFYGYHMHWVRQAPGQGLEWMGFI |
| ID | TRBC | NPYTNDIQYNERFRGRVTITSDESTTTAYMELSSLRSEDTAVYYCAMGNGKWG |
| NO: | 2- | DGAYRFFDLWGQGTLVTVSSASTKGPSVFPLAPSSKSTSGGTAALGCLVKDYFP |
| 4682 | BKM0238- | EPVTVSWNSGALTSGVHTFPAVLQSSGLYSLSSVVTVPSSSLGTQTYICNVNHK |
| | HC 1 | PSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYASTYRVVSVLTVLHQ |
| | | DWLNGKEYKCKVSNKALPAPIEKTISKAKGQPREPQVYTLPPCREEMTKNQVS |
| | | LWCLVKGFYPSDIAVEWESNGQPENNYKTTPPVLDSDGSFFLYSKLTVDKSRW |
| | | QQGNVFSCSVMHEALHNHYTQKSLSLSP |
| SEQ | Anti- | DVVMTQSPLSLPVTLGQPASISCRSSENLVHSNGRTYLQWYQQRPGQSPRLLIY |
| ID | TRBC | RVSNRFPGVPDRFSGSGSGTDFTLKISRVEAEDVGVYFCSQSSLEPYTFGGGTK |
| NO: | 2 - | VEIKRTVAAPSVFIFPPSDEQLKSGTASVVCLLNNFYPREAKVQWKVDNALQSG |
| 8002 | BKM0238- | NSQESVTEQDSKDSTYSLSSTLTLSKADYEKHKVYACEVTHQGLSSPVTKSFNR |
| | LC | GEC |
| SEQ | Nk- | EIQLLESGGGLVQPGGSLRLSCAVSGFSITTTGYHWNWVRQAPGKGLEWVGYI |
| ID | p30 | YSSGSTSYNPSLKSRFTISRDTSKNTFYLQMNSLRAEDTAVYYCARGDWHYFD |
| NO: | scFv | YWGQGTMVTVSSGGGGSGGGGSGGGGSGGGGSDSVTTQSPLSLPVTLGQPASI |
| 7383/ | | SCSGEKLSDKYVHWYQQRPGQSPRMLIYENDRRPSGVPDRFSGSNSGNDATLK |
| SEQ ID | | ISRVEAEDVGVYFCQFWDSTNSAVFGGGTKVEIKDKTHTCPPCPAPELLGGPSV |
| NO: | | FLFPPKPKDTLMISRTPEVTCVVVDVSHEDPEVKFNWYVDGVEVHNAKTKPRE |
| 8006 | | EQYASTYRVVSVLTVLHQDWLNGKEYKCKVSNKALPAPIEKTISKAKGQPREP |
| | | QVCTLPPSREEMTKNQVSLSCAVKGFYPSDIAVEWESNGQPENNYKTTPPVLDS |
| | | DGSFFLVSKLTVDKSRWQQGNVFSCSVMHEALHNHYTQKSLSLSPGK |
| SEQ | Nk- | EIQLLESGGGLVQPGGSLRLSCAVSGFSITTTGYHWNWVRQAPGKGLEWVGYI |
| ID | p30 | YSSGSTSYNPSLKSRFTISRDTSKNTFYLQMNSLRAEDTAVYYCARGDWHYFD |
| NO: | scFv | YWGQGTMVTVSSGGGGSGGGGSGGGGSGGGGSDSVTTQSPLSLPVTLGQPASI |
| 4684 | | SCSGEKLSDKYVHWYQQRPGQSPRMLIYENDRRPSGVPDRFSGSNSGNDATLK |
| | | ISRVEAEDVGVYFCQFWDSTNSAVFGGGTKVEIKDKTHTCPPCPAPELLGGPSV |
| | | FLFPPKPKDTLMISRTPEVTCVVVDVSHEDPEVKFNWYVDGVEVHNAKTKPRE |
| | | EQYASTYRVVSVLTVLHQDWLNGKEYKCKVSNKALPAPIEKTISKAKGQPREP |
| | | QVCTLPPSREEMTKNQVSLSCAVKGFYPSDIAVEWESNGQPENNYKTTPPVLDS |
| | | DGSFFLVSKLTVDKSRWQQGNVFSCSVMHEALHNHYTQKSLSLSP |

anti-TRBC2-NKp30 - BIS-2020-3

| | | |
|---|---|---|
| SEQ | Anti- | QVQLVQSGAEVKKPGSSVKVSCKASPRGFYGYHMHWVRQAPGQGLEWMGFI |
| ID | TRBC | NPYNNHIQYNERFRGRVTITSDESTTTAYMELSSLRSEDTAVYYCALGNGKWG |
| NO: | 2 - | DGAYRFFDFWGQGTLVTVSSASTKGPSVFPLAPSSKSTSGGTAALGCLVKDYFP |
| 8004 | BKM0240- | EPVTVSWNSGALTSGVHTFPAVLQSSGLYSLSSVVTVPSSSLGTQTYICNVNHK |
| | HC | PSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYASTYRVVSVLTVLHQ |
| | | DWLNGKEYKCKVSNKALPAPIEKTISKAKGQPREPQVYTLPPCREEMTKNQVS |
| | | LWCLVKGFYPSDIAVEWESNGQPENNYKTTPPVLDSDGSFFLYSKLTVDKSRW |
| | | QQGNVFSCSVMHEALHNHYTQKSLSLSPGK |
| SEQ | Anti- | QVQLVQSGAEVKKPGSSVKVSCKASPRGFYGYHMHWVRQAPGQGLEWMGFI |
| ID | TRBC | NPYNNHIQYNERFRGRVTITSDESTTTAYMELSSLRSEDTAVYYCALGNGKWG |
| NO: | 2 - | DGAYRFFDFWGQGTLVTVSSASTKGPSVFPLAPSSKSTSGGTAALGCLVKDYFP |
| 4685 | BKM0240- | EPVTVSWNSGALTSGVHTFPAVLQSSGLYSLSSVVTVPSSSLGTQTYICNVNHK |
| | HC | PSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYASTYRVVSVLTVLHQ |
| | | DWLNGKEYKCKVSNKALPAPIEKTISKAKGQPREPQVYTLPPCREEMTKNQVS |
| | | LWCLVKGFYPSDIAVEWESNGQPENNYKTTPPVLDSDGSFFLYSKLTVDKSRW |
| | | QQGNVFSCSVMHEALHNHYTQKSLSLSP |
| SEQ | Anti- | DVVMTQSPLSLPVTLGQPASISCRSSKNLVHSNGRTYLQWYQQRPGQSPRLLIY |
| ID | TRBC | RVSNRFPGVPDRFSGSGSGTDFTLKISRVEAEDVGVYFCSQSTREPYTFGGGTK |
| NO: | 2 -- | VEIKRTVAAPSVFIFPPSDEQLKSGTASVVCLLNNFYPREAKVQWKVDNALQSG |
| 8005 | BKM0240- | NSQESVTEQDSKDSTYSLSSTLTLSKADYEKHKVYACEVTHQGLSSPVTKSFNR |
| | LC | GEC |
| SEQ | Nk- | EIQLLESGGGLVQPGGSLRLSCAVSGFSITTTGYHWNWVRQAPGKGLEWVGYI |
| ID | p30 | YSSGSTSYNPSLKSRFTISRDTSKNTFYLQMNSLRAEDTAVYYCARGDWHYFD |
| NO: | scFv | YWGQGTMVTVSSGGGGSGGGGSGGGGSGGGGSDSVTTQSPLSLPVTLGQPASI |
| 8693/ | | SCSGEKLSDKYVHWYQQRPGQSPRMLIYENDRRPSGVPDRFSGSNSGNDATLK |
| SEQ | | ISRVEAEDVGVYFCQFWDSTNSAVFGGGTKVEIKDKTHTCPPCPAPELLGGPSV |
| ID | | FLFPPKPKDTLMISRTPEVTCVVVDVSHEDPEVKFNWYVDGVEVHNAKTKPRE |
| NO: | | EQYASTYRVVSVLTVLHQDWLNGKEYKCKVSNKALPAPIEKTISKAKGQPREP |
| 8003 | | QVCTLPPSREEMTKNQVSLSCAVKGFYPSDIAVEWESNGQPENNYKTTPPVLDS |
| | | DGSFFLVSKLTVDKSRWQQGNVFSCSVMHEALHNHYTQKSLSLSPGK |
| SEQ | Nk- | EIQLLESGGGLVQPGGSLRLSCAVSGFSITTTGYHWNWVRQAPGKGLEWVGYI |
| ID | p30 | YSSGSTSYNPSLKSRFTISRDTSKNTFYLQMNSLRAEDTAVYYCARGDWHYFD |
| NO: | scFv | YWGQGTMVTVSSGGGGSGGGGSGGGGSGGGGSDSVTTQSPLSLPVTLGQPASI |
| 4683 | | SCSGEKLSDKYVHWYQQRPGQSPRMLIYENDRRPSGVPDRFSGSNSGNDATLK |
| | | ISRVEAEDVGVYFCQFWDSTNSAVFGGGTKVEIKDKTHTCPPCPAPELLGGPSV |
| | | FLFPPKPKDTLMISRTPEVTCVVVDVSHEDPEVKFNWYVDGVEVHNAKTKPRE |
| | | EQYASTYRVVSVLTVLHQDWLNGKEYKCKVSNKALPAPIEKTISKAKGQPREP |
| | | QVCTLPPSREEMTKNQVSLSCAVKGFYPSDIAVEWESNGQPENNYKTTPPVLDS |
| | | DGSFFLVSKLTVDKSRWQQGNVFSCSVMHEALHNHYTQKSLSLSP |

anti-TRBC2-NKp30 - BIS-2020-4

| | | |
|---|---|---|
| SEQ | Anti- | QVQLVQSGAEVKKPGSSVKVSCKASPRGFYGYHMHWVRQAPGQGLEWMGFI |
| ID | TRBC | NPYNNHIQYNERFRGRVTITSDESTTTAYMELSSLRSEDTAVYYCALGNGKWG |
| NO: | 2 -- | DGAYRFFDFWGQGTLVTVSSASTKGPSVFPLAPSSKSTSGGTAALGCLVKDYFP |
| 8004 | BKM0240- | EPVTVSWNSGALTSGVHTFPAVLQSSGLYSLSSVVTVPSSSLGTQTYICNVNHK |
| | HC | PSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYASTYRVVSVLTVLHQ |
| | | DWLNGKEYKCKVSNKALPAPIEKTISKAKGQPREPQVYTLPPCREEMTKNQVS |
| | | LWCLVKGFYPSDIAVEWESNGQPENNYKTTPPVLDSDGSFFLYSKLTVDKSRW |
| | | QQGNVFSCSVMHEALHNHYTQKSLSLSPGK |
| SEQ | Anti- | QVQLVQSGAEVKKPGSSVKVSCKASPRGFYGYHMHWVRQAPGQGLEWMGFI |
| ID | TRBC | NPYNNHIQYNERFRGRVTITSDESTTTAYMELSSLRSEDTAVYYCALGNGKWG |
| NO: | 2 -- | DGAYRFFDFWGQGTLVTVSSASTKGPSVFPLAPSSKSTSGGTAALGCLVKDYFP |
| 4685 | BKM0240- | EPVTVSWNSGALTSGVHTFPAVLQSSGLYSLSSVVTVPSSSLGTQTYICNVNHK |
| | HC | PSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYASTYRVVSVLTVLHQ |
| | | DWLNGKEYKCKVSNKALPAPIEKTISKAKGQPREPQVYTLPPCREEMTKNQVS |
| | | LWCLVKGFYPSDIAVEWESNGQPENNYKTTPPVLDSDGSFFLYSKLTVDKSRW |
| | | QQGNVFSCSVMHEALHNHYTQKSLSLSP |
| SEQ | Anti- | DVVMTQSPLSLPVTLGQPASISCRSSKNLVHSNGRTYLQWYQQRPGQSPRLLIY |
| ID | TRBC | RVSNRFPGVPDRFSGSGSGTDFTLKISRVEAEDVGVYFCSQSTREPYTFGGGTK |
| NO: | 2- | VEIKRTVAAPSVFIFPPSDEQLKSGTASVVCLLNNFYPREAKVQWKVDNALQSG |
| 8005 | BKM0240- | NSQESVTEQDSKDSTYSLSSTLTLSKADYEKHKVYACEVTHQGLSSPVTKSFNR |
| | LC | GEC |
| SEQ | Nk- | EIQLLESGGGLVQPGGSLRLSCAVSGFSITTTGYHWNWVRQAPGKGLEWVGYI |
| ID | p30 | YSSGSTSYNPSLKSRFTISRDTSKNTFYLQMNSLRAEDTAVYYCARGDWHYFD |
| NO: | scFv | YWGQGTMVTVSSGGGGSGGGGSGGGGSGGGGSDSVTTQSPLSLPVTLGQPASI |
| 7383/ | | SCSGEKLSDKYVHWYQQRPGQSPRMLIYENDRRPSGVPDRFSGSNSGNDATLK |
| SEQ ID | | ISRVEAEDVGVYFCQFWDSTNSAVFGGGTKVEIKDKTHTCPPCPAPELLGGPSV |
| NO: | | FLFPPKPKDTLMISRTPEVTCVVVDVSHEDPEVKFNWYVDGVEVHNAKTKPRE |
| 8006 | | EQYASTYRVVSVLTVLHQDWLNGKEYKCKVSNKALPAPIEKTISKAKGQPREP |
| | | QVCTLPPSREEMTKNQVSLSCAVKGFYPSDIAVEWESNGQPENNYKTTPPVLDS |
| | | DGSFFLVSKLTVDKSRWQQGNVFSCSVMHEALHNHYTQKSLSLSPGK |
| SEQ | Nk- | EIQLLESGGGLVQPGGSLRLSCAVSGFSITTTGYHWNWVRQAPGKGLEWVGYI |
| ID | p30 | YSSGSTSYNPSLKSRFTISRDTSKNTFYLQMNSLRAEDTAVYYCARGDWHYFD |
| NO: | scFv | YWGQGTMVTVSSGGGGSGGGGSGGGGSGGGGSDSVTTQSPLSLPVTLGQPASI |
| 4684 | | SCSGEKLSDKYVHWYQQRPGQSPRMLIYENDRRPSGVPDRFSGSNSGNDATLK |
| | | ISRVEAEDVGVYFCQFWDSTNSAVFGGGTKVEIKDKTHTCPPCPAPELLGGPSV |
| | | FLFPPKPKDTLMISRTPEVTCVVVDVSHEDPEVKFNWYVDGVEVHNAKTKPRE |
| | | EQYASTYRVVSVLTVLHQDWLNGKEYKCKVSNKALPAPIEKTISKAKGQPREP |
| | | QVCTLPPSREEMTKNQVSLSCAVKGFYPSDIAVEWESNGQPENNYKTTPPVLDS |
| | | DGSFFLVSKLTVDKSRWQQGNVFSCSVMHEALHNHYTQKSLSLSP |

anti-TRBC2-NKp30 - BIS-2020-5

| | | |
|---|---|---|
| SEQ | Anti- | QVQLVQSGAEVKKPGSSVKVSCKASPRGFYGYHMHWVRQAPGQGLEWMGFI |
| ID | TRBC | NPYNNHIQYNERFRGRVTITSDESTTTAYMELSSLRSEDTAVYYCALGEGKWG |
| NO: | 2- | DGAYRFFDFWGQGTLVTVSSASTKGPSVFPLAPSSKSTSGGTAALGCLVKDYFP |
| 8007 | 0311- | EPVTVSWNSGALTSGVHTFPAVLQSSGLYSLSSVVTVPSSSLGTQTYICNVNHK |
| | HC | PSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYASTYRVVSVLTVLHQ |
| | | DWLNGKEYKCKVSNKALPAPIEKTISKAKGQPREPQVYTLPPCREEMTKNQVS |
| | | LWCLVKGFYPSDIAVEWESNGQPENNYKTTPPVLDSDGSFFLYSKLTVDKSRW |
| | | QQGNVFSCSVMHEALHNHYTQKSLSLSPGK |
| SEQ | Anti- | QVQLVQSGAEVKKPGSSVKVSCKASPRGFYGYHMHWVRQAPGQGLEWMGFI |
| ID | TRBC | NPYNNHIQYNERFRGRVTITSDESTTTAYMELSSLRSEDTAVYYCALGEGKWG |
| NO: | 2- | DGAYRFFDFWGQGTLVTVSSASTKGPSVFPLAPSSKSTSGGTAALGCLVKDYFP |
| 4686 | 0311- | EPVTVSWNSGALTSGVHTFPAVLQSSGLYSLSSVVTVPSSSLGTQTYICNVNHK |
| | HC | PSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYASTYRVVSVLTVLHQ |
| | | DWLNGKEYKCKVSNKALPAPIEKTISKAKGQPREPQVYTLPPCREEMTKNQVS |
| | | LWCLVKGFYPSDIAVEWESNGQPENNYKTTPPVLDSDGSFFLYSKLTVDKSRW |
| | | QQGNVFSCSVMHEALHNHYTQKSLSLSP |
| SEQ | Anti- | DVVMTQSPLSLPVTLGQPASISCRSSKNLVHSNGRTYLQWYQQRPGQSPRLLIY |
| ID | TRBC | RVSNRFPGVPDRFSGSGSGTDFTLKISRVEAEDVGVYFCSQSTREPYTFGGGTK |
| NO: | 2- | VEIKRTVAAPSVFIFPPSDEQLKSGTASVVCLLNNFYPREAKVQWKVDNALQSG |
| 8005 | 0311- | NSQESVTEODSKDSTYSLSSTLTLSKADYEKHKVYACEVTHQGLSSPVTKSFNR |
| | LC | GEC |
| SEQ | Nk- | EIQLLESGGGLVQPGGSLRLSCAVSGFSITTTGYHWNWVRQAPGKGLEWVGYI |
| ID | p30 | YSSGSTSYNPSLKSRFTISRDTSKNTFYLQMNSLRAEDTAVYYCARGDWHYFD |
| NO: | scFv | YWGQGTMVTVSSGGGGSGGGGSGGGGSGGGGSSSETTQPPSVSVSPGQTASIT |
| 7384/ | | CSGEKLSDKYVHWYQQKPGQSPVMVIYENDRRPSGIPERFSGSNSGNTATLTIS |
| SEQ | | GTQAMDEADYFCQFWDSTNSAVFGGGTQLTVLDKTHTCPPCPAPELLGGPSVF |
| ID | | LFPPKPKDTLMISRTPEVTCVVVDVSHEDPEVKFNWYVDGVEVHNAKTKPREE |
| NO: | | QYASTYRVVSVLTVLHQDWLNGKEYKCKVSNKALPAPIEKTISKAKGQPREPQ |
| 8698 | | VCTLPPSREEMTKNQVSLSCAVKGFYPSDIAVEWESNGQPENNYKTTPPVLDSD |
| | | GSFFLVSKLTVDKSRWQQGNVFSCSVMHEALHNHYTQKSLSLSPGK |
| SEQ | Nk- | EIQLLESGGGLVQPGGSLRLSCAVSGFSITTTGYHWNWVRQAPGKGLEWVGYI |
| ID | p30 | YSSGSTSYNPSLKSRFTISRDTSKNTFYLQMNSLRAEDTAVYYCARGDWHYFD |
| NO: | scFv | YWGQGTMVTVSSGGGGSGGGGSGGGGSGGGGSSSETTQPPSVSVSPGQTASIT |
| 8690 | | CSGEKLSDKYVHWYQQKPGQSPVMVIYENDRRPSGIPERFSGSNSGNTATLTIS |
| | | GTQAMDEADYFCQFWDSTNSAVFGGGTQLTVLDKTHTCPPCPAPELLGGPSVF |
| | | LFPPKPKDTLMISRTPEVTCVVVDVSHEDPEVKFNWYVDGVEVHNAKTKPREE |
| | | QYASTYRVVSVLTVLHQDWLNGKEYKCKVSNKALPAPIEKTISKAKGQPREPQ |
| | | VCTLPPSREEMTKNQVSLSCAVKGFYPSDIAVEWESNGQPENNYKTTPPVLDSD |
| | | GSFFLVSKLTVDKSRWQQGNVFSCSVMHEALHNHYTQKSLSLSP |

anti-TRBC2-NKp30 - BIS-2020-6

| | | |
|---|---|---|
| SEQ | Anti- | QVQLVQSGAEVKKPGSSVKVSCKASPRGFYGYHMHWVRQAPGQGLEWMGFI |
| ID | TRBC | NPYNNHIQYNERFRGRVTITSDESTTTAYMELSSLRSEDTAVYYCALGEGKWG |
| NO: | 2- | DGAYRFFDFWGQGTLVTVSSASTKGPSVFPLAPSSKSTSGGTAALGCLVKDYFP |
| 8007 | 0311- | EPVTVSWNSGALTSGVHTFPAVLQSSGLYSLSSVVTVPSSSLGTQTYICNVNHK |
| | HC | PSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYASTYRVVSVLTVLHQ |
| | | DWLNGKEYKCKVSNKALPAPIEKTISKAKGQPREPQVYTLPPCREEMTKNQVS |
| | | LWCLVKGFYPSDIAVEWESNGQPENNYKTTPPVLDSDGSFFLYSKLTVDKSRW |
| | | QQGNVFSCSVMHEALHNHYTQKSLSLSPGK |
| SEQ | Anti- | QVQLVQSGAEVKKPGSSVKVSCKASPRGFYGYHMHWVRQAPGQGLEWMGFI |
| ID | TRBC | NPYNNHIQYNERFRGRVTITSDESTTTAYMELSSLRSEDTAVYYCALGEGKWG |
| NO: | 2- | DGAYRFFDFWGQGTLVTVSSASTKGPSVFPLAPSSKSTSGGTAALGCLVKDYFP |
| 4686 | 0311- | EPVTVSWNSGALTSGVHTFPAVLQSSGLYSLSSVVTVPSSSLGTQTYICNVNHK |
| | HC | PSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYASTYRVVSVLTVLHQ |
| | | DWLNGKEYKCKVSNKALPAPIEKTISKAKGQPREPQVYTLPPCREEMTKNQVS |
| | | LWCLVKGFYPSDIAVEWESNGQPENNYKTTPPVLDSDGSFFLYSKLTVDKSRW |
| | | QQGNVFSCSVMHEALHNHYTQKSLSLSP |
| SEQ | Anti- | DVVMTQSPLSLPVTLGQPASISCRSSKNLVHSNGRTYLQWYQQRPGQSPRLLIY |
| ID | TRBC | RVSNRFPGVPDRFSGSGSGTDFTLKISRVEAEDVGVYFCSQSTREPYTFGGGTK |
| NO: | 2- | VEIKRTVAAPSVFIFPPSDEQLKSGTASVVCLLNNFYPREAKVQWKVDNALQSG |
| 8005 | 0311- | NSQESVTEQDSKDSTYSLSSTLTLSKADYEKHKVYACEVTHQGLSSPVTKSFNR |
| | LC | GEC |
| SEQ | Nk- | EIQLLESGGGLVQPGGSLRLSCAVSGFSITTTGYHWNWVRQAPGKGLEWVGYI |
| ID | p30 | YSSGSTSYNPSLKSRFTISRDTSKNTFYLQMNSLRAEDTAVYYCARGDWHYFD |
| NO: | scFv | YWGQGTMVTVSSGGGGSGGGGSGGGGSGGGGSDSVTTQSPLSLPVTLGQPASI |
| 7383/ | | SCSGEKLSDKYVHWYQQRPGQSPRMLIYENDRRPSGVPDRFSGSNSGNDATLK |
| SEQ ID | | ISRVEAEDVGVYFCQFWDSTNSAVFGGGTKVEIKDKTHTCPPCPAPELLGGPSV |
| NO: | | FLFPPKPKDTLMISRTPEVTCVVVDVSHEDPEVKFNWYVDGVEVHNAKTKPRE |
| 8006 | | EQYASTYRVVSVLTVLHQDWLNGKEYKCKVSNKALPAPIEKTISKAKGQPREP |
| | | QVCTLPPSREEMTKNQVSLSCAVKGFYPSDIAVEWESNGQPENNYKTTPPVLDS |
| | | DGSFFLVSKLTVDKSRWQQGNVFSCSVMHEALHNHYTQKSLSLSPGK |
| SEQ | Nk- | EIQLLESGGGLVQPGGSLRLSCAVSGFSITTTGYHWNWVRQAPGKGLEWVGYI |
| ID | p30 | YSSGSTSYNPSLKSRFTISRDTSKNTFYLQMNSLRAEDTAVYYCARGDWHYFD |
| NO: | scFv | YWGQGTMVTVSSGGGGSGGGGSGGGGSGGGGSDSVTTQSPLSLPVTLGQPASI |
| 4684 | | SCSGEKLSDKYVHWYQQRPGQSPRMLIYENDRRPSGVPDRFSGSNSGNDATLK |
| | | ISRVEAEDVGVYFCQFWDSTNSAVFGGGTKVEIKDKTHTCPPCPAPELLGGPSV |
| | | FLFPPKPKDTLMISRTPEVTCVVVDVSHEDPEVKFNWYVDGVEVHNAKTKPRE |
| | | EQYASTYRVVSVLTVLHQDWLNGKEYKCKVSNKALPAPIEKTISKAKGQPREP |
| | | QVCTLPPSREEMTKNQVSLSCAVKGFYPSDIAVEWESNGQPENNYKTTPPVLDS |
| | | DGSFFLVSKLTVDKSRWQQGNVFSCSVMHEALHNHYTQKSLSLSP |

anti-TRBC2-NKp30 - BIS-2020-7

| | | |
|---|---|---|
| SEQ | Anti- | QVQLVQSGAEVKKPGSSVKVSCKASPRGFYGYHMHWVRQAPGQGLEWMGFI |
| ID | TRBC | NPYNNHIQYNERFRGRVTITSDESTTTAYMELSSLRSEDTAVYYCALGAGKWG |
| NO: | 2- | DGAYRFFDFWGQGTLVTVSSASTKGPSVFPLAPSSKSTSGGTAALGCLVKDYFP |
| 8009 | 0314- | EPVTVSWNSGALTSGVHTFPAVLQSSGLYSLSSVVTVPSSSLGTQTYICNVNHK |
| | HC | PSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYASTYRVVSVLTVLHQ |
| | | DWLNGKEYKCKVSNKALPAPIEKTISKAKGQPREPQVYTLPPCREEMTKNQVS |
| | | LWCLVKGFYPSDIAVEWESNGQPENNYKTTPPVLDSDGSFFLYSKLTVDKSRW |
| | | QQGNVFSCSVMHEALHNHYTQKSLSLSPGK |
| SEQ | Anti- | QVQLVQSGAEVKKPGSSVKVSCKASPRGFYGYHMHWVRQAPGQGLEWMGFI |
| ID | TRBC | NPYNNHIQYNERFRGRVTITSDESTTTAYMELSSLRSEDTAVYYCALGAGKWG |
| NO: | 2- | DGAYRFFDFWGQGTLVTVSSASTKGPSVFPLAPSSKSTSGGTAALGCLVKDYFP |
| 4687 | 0314- | EPVTVSWNSGALTSGVHTFPAVLQSSGLYSLSSVVTVPSSSLGTQTYICNVNHK |
| | HC | PSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYASTYRVVSVLTVLHQ |
| | | DWLNGKEYKCKVSNKALPAPIEKTISKAKGQPREPQVYTLPPCREEMTKNQVS |
| | | LWCLVKGFYPSDIAVEWESNGQPENNYKTTPPVLDSDGSFFLYSKLTVDKSRW |
| | | QQGNVFSCSVMHEALHNHYTQKSLSLSP |
| SEQ | Anti- | DVVMTQSPLSLPVTLGQPASISCRSSKNLVHSNGRTYLQWYQQRPGQSPRLLIY |
| ID | TRBC | RVSNRFPGVPDRFSGSGSGTDFTLKISRVEAEDVGVYFCSQSTREPYTFGGGTK |
| NO: | 2- | VEIKRTVAAPSVFIFPPSDEQLKSGTASVVCLLNNFYPREAKVQWKVDNALQSG |
| 8005 | 0314- | NSQESVTEQDSKDSTYSLSSTLTLSKADYEKHKVYACEVTHQGLSSPVTKSFNR |
| | LC | GEC |
| SEQ | Nk- | EIQLLESGGGLVQPGGSLRLSCAVSGFSITTTGYHWNWVRQAPGKGLEWVGYI |
| ID | p30 | YSSGSTSYNPSLKSRFTISRDTSKNTFYLQMNSLRAEDTAVYYCARGDWHYFD |
| NO: | scFv | YWGQGTMVTVSSGGGGSGGGGSGGGGSGGGGSSSETTQPPSVSVSPGQTASIT |
| 7384/ | | CSGEKLSDKYVHWYQQKPGQSPVMVIYENDRRPSGIPERFSGSNSGNTATLTIS |
| SEQ | | GTQAMDEADYFCQFWDSTNSAVFGGGTQLTVLDKTHTCPPCPAPELLGGPSVF |
| ID | | LFPPKPKDTLMISRTPEVTCVVVDVSHEDPEVKFNWYVDGVEVHNAKTKPREE |
| NO: | | QYASTYRVVSVLTVLHQDWLNGKEYKCKVSNKALPAPIEKTISKAKGQPREPQ |
| 8698 | | VCTLPPSREEMTKNQVSLSCAVKGFYPSDIAVEWESNGQPENNYKTTPPVLDSD |
| | | GSFFLVSKLTVDKSRWQQGNVFSCSVMHEALHNHYTQKSLSLSPGK |
| SEQ | Nk- | EIQLLESGGGLVQPGGSLRLSCAVSGFSITTTGYHWNWVRQAPGKGLEWVGYI |
| ID | p30 | YSSGSTSYNPSLKSRFTISRDTSKNTFYLQMNSLRAEDTAVYYCARGDWHYFD |
| NO: | scFv | YWGQGTMVTVSSGGGGSGGGGSGGGGSGGGGSSSETTQPPSVSVSPGQTASIT |
| 8690 | | CSGEKLSDKYVHWYQQKPGQSPVMVIYENDRRPSGIPERFSGSNSGNTATLTIS |
| | | GTQAMDEADYFCQFWDSTNSAVFGGGTQLTVLDKTHTCPPCPAPELLGGPSVF |
| | | LFPPKPKDTLMISRTPEVTCVVVDVSHEDPEVKFNWYVDGVEVHNAKTKPREE |
| | | QYASTYRVVSVLTVLHQDWLNGKEYKCKVSNKALPAPIEKTISKAKGQPREPQ |
| | | VCTLPPSREEMTKNQVSLSCAVKGFYPSDIAVEWESNGQPENNYKTTPPVLDSD |
| | | GSFFLVSKLTVDKSRWQQGNVFSCSVMHEALHNHYTQKSLSLSP |

anti-TRBC2-NKp30 - BIS-2020-8

| | | |
|---|---|---|
| SEQ | Anti- | QVQLVQSGAEVKKPGSSVKVSCKASPRGFYGYHMHWVRQAPGQGLEWMGFI |
| ID | TRBC | NPYNNHIQYNERFRGRVTITSDESTTTAYMELSSLRSEDTAVYYCALGAGKWG |
| NO: | 2- | DGAYRFFDFWGQGTLVTVSSASTKGPSVFPLAPSSKSTSGGTAALGCLVKDYFP |
| 8009 | 0314- | EPVTVSWNSGALTSGVHTFPAVLQSSGLYSLSSVVTVPSSSLGTQTYICNVNHK |
| | HC | PSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYASTYRVVSVLTVLHQ |
| | | DWLNGKEYKCKVSNKALPAPIEKTISKAKGQPREPQVYTLPPCREEMTKNQVS |
| | | LWCLVKGFYPSDIAVEWESNGQPENNYKTTPPVLDSDGSFFLYSKLTVDKSRW |
| | | QQGNVFSCSVMHEALHNHYTQKSLSLSPGK |
| SEQ | Anti- | QVQLVQSGAEVKKPGSSVKVSCKASPRGFYGYHMHWVRQAPGQGLEWMGFI |
| ID | TRBC | NPYNNHIQYNERFRGRVTITSDESTTTAYMELSSLRSEDTAVYYCALGAGKWG |
| NO: | 2- | DGAYRFFDFWGQGTLVTVSSASTKGPSVFPLAPSSKSTSGGTAALGCLVKDYFP |
| 4687 | 0314- | EPVTVSWNSGALTSGVHTFPAVLQSSGLYSLSSVVTVPSSSLGTQTYICNVNHK |
| | HC | PSNTKVDKRVEPKSCDKTHTCPPCPAPELLGGPSVFLFPPKPKDTLMISRTPEVT |
| | | CVVVDVSHEDPEVKFNWYVDGVEVHNAKTKPREEQYASTYRVVSVLTVLHQ |
| | | DWLNGKEYKCKVSNKALPAPIEKTISKAKGQPREPQVYTLPPCREEMTKNQVS |
| | | LWCLVKGFYPSDIAVEWESNGQPENNYKTTPPVLDSDGSFFLYSKLTVDKSRW |
| | | QQGNVFSCSVMHEALHNHYTQKSLSLSP |
| SEQ | Anti- | DVVMTQSPLSLPVTLGQPASISCRSSKNLVHSNGRTYLQWYQQRPGQSPRLLIY |
| ID | TRBC | RVSNRFPGVPDRFSGSGSGTDFTLKISRVEAEDVGVYFCSQSTREPYTFGGGTK |
| NO: | 2- | VEIKRTVAAPSVFIFPPSDEQLKSGTASVVCLLNNFYPREAKVQWKVDNALQSG |
| 8005 | 0314- | NSQESVTEQDSKDSTYSLSSTLTLSKADYEKHKVYACEVTHQGLSSPVTKSFNR |
| | LC | GEC |
| SEQ | Nk- | EIQLLESGGGLVQPGGSLRLSCAVSGFSITTTGYHWNWVRQAPGKGLEWVGYI |
| ID | p30 | YSSGSTSYNPSLKSRFTISRDTSKNTFYLQMNSLRAEDTAVYYCARGDWHYFD |
| NO: | scFv | YWGQGTMVTVSSGGGGSGGGGSGGGGSGGGGSDSVTTQSPLSLPVTLGQPASI |
| 7383/ | | SCSGEKLSDKYVHWYQQRPGQSPRMLIYENDRRPSGVPDRFSGSNSGNDATLK |
| SEQ ID | | ISRVEAEDVGVYFCQFWDSTNSAVFGGGTKVEIKDKTHTCPPCPAPELLGGPSV |
| NO: | | FLFPPKPKDTLMISRTPEVTCVVVDVSHEDPEVKFNWYVDGVEVHNAKTKPRE |
| 8006 | | EQYASTYRVVSVLTVLHQDWLNGKEYKCKVSNKALPAPIEKTISKAKGQPREP |
| | | QVCTLPPSREEMTKNQVSLSCAVKGFYPSDIAVEWESNGQPENNYKTTPPVLDS |
| | | DGSFFLVSKLTVDKSRWQQGNVFSCSVMHEALHNHYTQKSLSLSPGK |
| SEQ | Nk- | EIQLLESGGGLVQPGGSLRLSCAVSGFSITTTGYHWNWVRQAPGKGLEWVGYI |
| ID | p30 | YSSGSTSYNPSLKSRFTISRDTSKNTFYLQMNSLRAEDTAVYYCARGDWHYFD |
| NO: | scFv | YWGQGTMVTVSSGGGGSGGGGSGGGGSGGGGSDSVTTQSPLSLPVTLGQPASI |
| 4684 | | SCSGEKLSDKYVHWYQQRPGQSPRMLIYENDRRPSGVPDRFSGSNSGNDATLK |
| | | ISRVEAEDVGVYFCQFWDSTNSAVFGGGTKVEIKDKTHTCPPCPAPELLGGPSV |
| | | FLFPPKPKDTLMISRTPEVTCVVVDVSHEDPEVKFNWYVDGVEVHNAKTKPRE |
| | | EQYASTYRVVSVLTVLHQDWLNGKEYKCKVSNKALPAPIEKTISKAKGQPREP |
| | | QVCTLPPSREEMTKNQVSLSCAVKGFYPSDIAVEWESNGQPENNYKTTPPVLDS |
| | | DGSFFLVSKLTVDKSRWQQGNVFSCSVMHEALHNHYTQKSLSLSP |

TABLE 20A
Exemplary heavy chain CDRs and FWRs of NKp30-targeting antigen
binding domains (According to Kabat numbering scheme)

| Ab ID | VHFWR1 | VHCDR1 | VHFWR2 | VHCDR2 | VHFWR3 | VHCDR3 | VHFWR4 |
|---|---|---|---|---|---|---|---|
| 9G1- | QIQLQESG | GYHWN | WIRQFP | YIYSSGS | RISITRDT | GNWHYF | WGQGTM |
| HC | PGLVKPSQ | (SEQ | GKKLEW | TSYNPSL | SKNQFFLQ | DF(SEQ | VTVSS |
| | SLSLTCSV | ID NO: | MG | KS(SEQ | LNSVTTED | ID NO: | (SEQ ID |
| | TGFSINTG | 6000) | (SEQ | ID NO: | TATYYCAR | 6002) | NO: 304) |
| | (SEQ ID | | ID NO: | 6001) | (SEQ ID | | |
| | NO: 6003) | | 6004) | | NO: 6005) | | |
| 15H6- | QIQLQESG | GYHWN | WIRQFP | YIYSSGT | RISITRDT | GNWHYF | WGQGTL |
| HC | PGLVKPSQ | (SEQ | GKKLEW | TRYNPSL | SKNQFFLQ | DY(SEQ | VAVSS |
| | SLSLTCSV | ID NO: | MG | KS(SEQ | LNSVTPED | ID NO: | (SEQ ID |
| | TGFSINTG | 6007) | (SEQ | ID NO: | TATYYCTR | 6009) | NO: |
| | (SEQ ID | | ID NO: | 6008) | (SEQ ID | | 6013) |
| | NO: 6010) | | 6011) | | NO: 6012) | | |
| 9G1- | QIQLQESG | GYHWN | WIRQPA | YIYSSGS | RVTMSRDT | GNWHYF | WGQGTM |
| HC_1 | PGLVKPSE | (SEQ | GKGLEW | TSYNPSL | SKNQFSLK | DF(SEQ | VTVSS |
| | TLSLTCTV | ID NO: | IG | KS(SEQ | LSSVTAAD | ID NO: | (SEQ ID |
| | SGFSINTG | 6000) | (SEQ | ID NO: | TAVYYCAR | 6002) | NO: 304) |
| | (SEQ ID | | ID NO: | 6001) | (SEQ ID | | |
| | NO: 6014) | | 6015) | | NO: 6016) | | |
| 9G1- | QIQLQESG | GYHWN | WIRQHP | YIYSSGS | LVTISRDT | GNWHYF | WGQGTM |
| HC_2 | PGLVKPSQ | (SEQ | GKGLEW | TSYNPSL | SKNQFSLK | DF(SEQ | VTVSS |
| | TLSLTCTV | ID NO: | IG | KS(SEQ | LSSVTAAD | ID NO: | (SEQ ID |
| | SGFSINTG | 6000) | (SEQ | ID NO: | TAVYYCAR | 6002) | NO: 304) |
| | (SEQ ID | | ID NO: | 6001) | (SEQ ID | | |
| | NO: 6018) | | 6019) | | NO: 6020) | | |
| 9G1- | EIQLLESG | GYHWN | WVRQAP | YIYSSGS | RFTISRDT | GNWHYF | WGQGTM |
| HC_3 | GGLVQPGG | (SEQ | GKGLEW | TSYNPSL | SKNTFYLQ | DF(SEQ | VTVSS |
| | SLRLSCAV | ID NO: | VG | KS(SEQ | MNSLRAED | ID NO: | (SEQ ID |
| | SGFSINTG | 6000) | (SEQ | ID NO: | TAVYYCAR | 6002) | NO: 304) |
| | (SEQ ID | | ID NO: | 6001) | (SEQ ID | | |
| | NO: 6022) | | 6023) | | NO: 316) | | |
| 9G1- | QIQLVQSG | GYHWN | WVRQAP | YIYSSGS | RVTITRDT | GNWHYF | WGQGTM |
| HC_4 | AEVKKPGS | (SEQ | GQGLEW | TSYNPSL | STNTFYME | DF(SEQ | VTVSS |
| | SVKVSCKV | ID NO: | MG | KS(SEQ | LSSLRSED | ID NO: | (SEQ ID |
| | SGFSINTG | 6000) | (SEQ | ID NO: | TAVYYCAR | 6002) | NO: 304) |
| | (SEQ ID | | ID NO: | 6001) | (SEQ ID | | |
| | NO: 6026) | | 6027) | | NO: 6028) | | |
| 9G1- | EIQLVESG | GYHWN | WVRQAP | YIYSSGS | RFTISRDT | GNWHYF | WGQGTM |
| HC_5 | GGLVQPGG | (SEQ | GKGLEW | TSYNPSL | AKNSFYLQ | DF(SEQ | VTVSS |
| | SLRLSCAV | ID NO: | VG | KS(SEQ | MNSLRAED | ID NO: | (SEQ ID |
| | SGFSINTG | 6000) | (SEQ | ID NO: | TAVYYCAR | 6002) | NO: 304) |
| | (SEQ ID | | ID NO: | 6001) | (SEQ ID | | |
| | NO: 6030) | | 6032) | | NO: 6033) | | |
| 9G1- | QIQLVQSG | GYHWN | WVRQAP | YIYSSGS | RVTMTRDT | GNWHYF | WGQGTM |
| HC_6 | AEVKKPGA | (SEQ | GQGLEW | TSYNPSL | STNTFYME | DF(SEQ | VTVSS |
| | SVKVSCKV | ID NO: | MG | KS(SEQ | LSSLRSED | ID NO: | (SEQ ID |
| | SGFSINTG | 6000) | (SEQ | ID NO: | TAVYYCAR | 6002) | NO: 304) |
| | (SEQ ID | | ID NO: | 6001) | (SEQ ID | | |
| | NO: 6035) | | 6036) | | NO: 6037) | | |
| 15H6- | QIQLQESG | GYHWN | WIRQHP | YIYSSGT | LVTISRDT | GNWHYF | WGQGTL |
| HC_1 | PGLVKPSQ | (SEQ | GKGLEW | TRYNPSL | SKNQFSLK | DY(SEQ | VTVSS |
| | TLSLTCTV | ID NO: | IG | KS(SEQ | LSSVTAAD | ID NO: | (SEQ ID |
| | SGFSINTG | 6007) | (SEQ | ID NO: | TAVYYCAR | 6009) | NO: |
| | (SEQ ID | | ID NO: | 6008) | (SEQ ID | | 6042) |
| | NO: 6039) | | 6040) | | NO: 6041) | | |
| 15H6- | QIQLQESG | GYHWN | WIRQPA | YIYSSGT | RVTMSRDT | GNWHYF | WGQGTL |
| HC_2 | PGLVKPSE | (SEQ | GKGLEW | TRYNPSL | SKNQFSLK | DY(SEQ | VTVSS |
| | TLSLTCTV | ID NO: | IG | KS(SEQ | LSSVTAAD | ID NO: | (SEQ ID |
| | SGFSINTG | 6007) | (SEQ | ID NO: | TAVYYCAR | 6009) | NO: |
| | (SEQ ID | | ID NO: | 6008) | (SEQ ID | | 6046) |
| | NO: 6043) | | 6044) | | NO: 6045) | | |
| 15H6- | EIQLLESG | GYHWN | WVRQAP | YIYSSGT | RFTISRDT | GNWHYF | WGQGTL |
| HC_3 | GGLVQPGG | (SEQ | GKGLEW | TRYNPSL | SKNTFYLQ | DY(SEQ | VTVSS |
| | SLRLSCAV | ID NO: | VG | KS(SEQ | MNSLRAED | ID NO: | (SEQ ID |
| | SGFSINTG | 6007) | (SEQ | ID NO: | TAVYYCAR | 6009) | NO: |
| | (SEQ ID | | ID NO: | 6008) | (SEQ ID | | 6050) |
| | NO: 6047) | | 6048) | | NO: 316) | | |
| 15H6- | QIQLVESG | GYHWN | WIRQAP | YIYSSGT | RFTISRDT | GNWHYF | WGQGTL |
| HC_4 | GGLVKPGG | (SEQ | GKGLEW | TRYNPSL | AKNSFYLQ | DY(SEQ | VTVSS |
| | SLRLSCAV | ID NO: | VG | KS(SEQ | MNSLRAED | ID NO: | (SEQ ID |
| | SGFSINTG | 6007) | (SEQ | ID NO: | TAVYYCAR | 6009) | NO: |
| | (SEQ ID | | ID NO: | 6008) | (SEQ ID | | 6054) |
| | NO: 6051) | | 6052) | | NO: 6053) | | |
| 15H6- | QIQLVQSG | GYHWN | WVRQAP | YIYSSGT | RVTMTRDT | GNWHYF | WGQGTL |
| HC_5 | AEVKKPGA | (SEQ | GQGLEW | TRYNPSL | STNTFYME | DY(SEQ | VTVSS |
| | SVKVSCKV | ID NO: | MG | KS(SEQ | LSSLRSED | ID NO: | (SEQ ID |
| | SGFSINTG | 6007) | (SEQ | ID NO: | TAVYYCAR | 6009) | NO: |
| | (SEQ ID | | ID NO: | 6008) | (SEQ ID | | 6058) |
| | NO: 6055) | | 6056) | | NO: 6057) | | |
| 15H6- | EIQLVQSG | GYHWN | WVQQAP | YIYSSGT | RVTITRDT | GNWHYF | WGQGTL |
| HC_6 | AEVKKPGA | (SEQ | GKGLEW | TRYNPSL | STNTFYME | DY(SEQ | VTVSS |
| | TVKISCKV | ID NO: | MG | KS(SEQ | LSSLRSED | ID NO: | (SEQ ID |
| | SGFSINTG | 6007) | (SEQ | ID NO: | TAVYYCAR | 6009) | NO: |
| | (SEQ ID | | ID NO: | 6008) | (SEQ ID | | 6062) |
| | NO: 6059) | | 6060) | | NO: 6061) | | |

TABLE 20B
Exemplary heavy chain CDRs and FWRs of NKp30-targeting antigen
binding domains of Table 20A (According to ABM numbering scheme)

| Ab ID | VHFWR1 | VHCDR1 | VHFWR2 | VHCDR2 | VHFWR3 | VHCDR3 | VHFWR4 |
|---|---|---|---|---|---|---|---|
| 9G1- | QIQLQESGP | GFSINT | WIRQFP | YIYSSGS | RISITRDT | GNWHYF | WGQGTM |
| HC | GLVKPSQSL | GGYHWN | GKKLEW | TSYNPSL | SKNQFFLQ | DF(SEQ | VTVSS |
| | SLTCSVT | (SEQ | MG | KS(SEQ | LNSVTTED | ID NO: | (SEQ ID |
| | (SEQ ID | ID NO: | (SEQ | ID NO: | TATYYCAR | 6002) | NO: 304) |
| | NO: 8553) | 8554) | ID NO: | 6001) | (SEQ ID | | |
| | | | 6004) | | NO: 6005) | | |
| 15H6- | QIQLQESGP | GFSINT | WIRQFP | YIYSSGT | RISITRDT | GNWHYF | WGQGTL |
| HC | GLVKPSQSL | GGYHWN | GKKLEW | TRYNPSL | SKNQFFLQ | DY(SEQ | VAVSS |
| | SLTCSVT | (SEQ | MG | KS(SEQ | LNSVTPED | ID NO: | (SEQ ID |
| | (SEQ ID | ID NO: | (SEQ | ID NO: | TATYYCTR | 6009) | NO: |
| | NO: 8555) | 8556) | ID NO: | 6008) | (SEQ ID | | 6013) |
| | | | 6011) | | NO: 6012) | | |
| 9G1- | QIQLQESGP | GFSINT | WIRQPA | YIYSSGS | RVTMSRDT | GNWHYF | WGQGTM |
| HC_1 | GLVKPSETL | GGYHWN | GKGLEW | TSYNPSL | SKNQFSLK | DF(SEQ | VTVSS |
| | SLTCTVS | (SEQ | IG | KS(SEQ | LSSVTAAD | ID NO: | (SEQ ID |
| | (SEQ ID | ID NO: | (SEQ | ID NO: | TAVYYCAR | 6002) | NO: 304) |
| | NO: 8557) | 8558) | ID NO: | 6001) | (SEQ ID | | |
| | | | 6015) | | NO: 6016) | | |
| 9G1- | QIQLQESGP | GFSINT | WIRQHP | YIYSSGS | LVTISRDT | GNWHYF | WGQGTM |
| HC_2 | GLVKPSQTL | GGYHWN | GKGLEW | TSYNPSL | SKNQFSLK | DF(SEQ | VTVSS |
| | SLTCTVS | (SEQ | IG | KS(SEQ | LSSVTAAD | ID NO: | (SEQ ID |
| | (SEQ ID | ID NO: | (SEQ | ID NO: | TAVYYCAR | 6002) | NO: 304) |
| | NO: 8559) | 8560) | ID NO: | 6001) | (SEQ ID | | |
| | | | 6019) | | NO: 6020) | | |
| 9G1- | EIQLLESGG | GFSINT | WVRQAP | YIYSSGS | RFTISRDT | GNWHYF | WGQGTM |
| HC_3 | GLVQPGGSL | GGYHWN | GKGLEW | TSYNPSL | SKNTFYLQ | DF(SEQ | VTVSS |
| | RLSCAVS | (SEQ | VG | KS(SEQ | MNSLRAED | ID NO: | (SEQ ID |
| | (SEQ ID | ID NO: | (SEQ | ID NO: | TAVYYCAR | 6002) | NO: 304) |
| | NO: 8561) | 8562) | ID NO: | 6001) | (SEQ ID | | |
| | | | 6023) | | NO: 316) | | |
| 9G1- | QIQLVQSGA | GFSINT | WVRQAP | YIYSSGS | RVTITRDT | GNWHYF | WGQGTM |
| HC_4 | EVKKPGSSV | GGYHWN | GQGLEW | TSYNPSL | STNTFYME | DF(SEQ | VTVSS |
| | KVSCKVS | (SEQ | MG | KS(SEQ | LSSLRSED | ID NO: | (SEQ ID |
| | (SEQ ID | ID NO: | (SEQ | ID NO: | TAVYYCAR | 6002) | NO: 304) |
| | NO: 8563) | 8564) | ID NO: | 6001) | (SEQ ID | | |
| | | | 6027) | | NO: 6028) | | |
| 9G1- | EIQLVESGG | GFSINT | WVRQAP | YIYSSGS | RFTISRDT | GNWHYF | WGQGTM |
| HC_5 | GLVQPGGSL | GGYHWN | GKGLEW | TSYNPSL | AKNSFYLQ | DF(SEQ | VTVSS |
| | RLSCAVS | (SEQ | VG | KS(SEQ | MNSLRAED | ID NO: | (SEQ ID |
| | (SEQ ID | ID NO: | (SEQ | ID NO: | ATVYYCAR | 6002) | NO: 304) |
| | NO: 8565) | 8566) | ID NO: | 6001) | (SEQ ID | | |
| | | | 6032) | | NO: 6033) | | |
| 9G1- | QIQLVQSGA | GFSINT | WVRQAP | YIYSSGS | RVTMTRDT | GNWHYF | WGQGTM |
| HC_6 | EVKKPGASV | GGYHWN | GQGLEW | TSYNPSL | STNTFYME | DF(SEQ | VTVSS |
| | KVSCKVS | (SEQ | MG | KS(SEQ | LSSLRSED | ID NO: | (SEQ ID |
| | (SEQ ID | ID NO: | (SEQ | ID NO: | TAVYYCAR | 6002) | NO: 304) |
| | NO: 8567) | 8568) | ID NO: | 6001) | (SEQ ID | | |
| | | | 6036) | | NO: 6037) | | |
| 15H6- | QIQLQESGP | GFSINT | WIRQHP | YIYSSGT | LVTISRDT | GNWHYF | WGQGTL |
| HC_1 | GLVKPSQTL | GGYHWN | GKGLEW | TRYNPSL | SKNQFSLK | DY(SEQ | VTVSS |
| | SLTCTVS | (SEQ | IG | KS(SEQ | LSSVTAAD | ID NO: | (SEQ ID |
| | (SEQ ID | ID NO: | (SEQ | ID NO: | TAVYYCAR | 6009) | NO: |
| | NO: 8569) | 8570) | ID NO: | 6008) | (SEQ ID | | 6042) |
| | | | 6040) | | NO: 6041) | | |
| 15H6- | QIQLQESGP | GFSINT | WIRQPA | YIYSSGT | RVTMSRDT | GNWHYF | WGQGTL |
| HC_2 | GLVKPSETL | GGYHWN | GKGLEW | TRYNPSL | SKNQFSLK | DY(SEQ | VTVSS |
| | SLTCTVS | (SEQ | IG | KS(SEQ | LSSVTAAD | ID NO: | (SEQ ID |
| | (SEQ ID | ID NO: | (SEQ | ID NO: | TAVYYCAR | 6009) | NO: |
| | NO: 8571) | 8572) | ID NO: | 6008) | (SEQ ID | | 6046) |
| | | | 6044) | | NO: 6045) | | |
| 15H6- | EIQLLESGG | GFSINT | WVRQAP | YIYSSGT | RFTISRDT | GNWHYF | WGQGTL |
| HC_3 | GLVQPGGSL | GGYHWN | GKGLEW | TRYNPSL | SKNTFYLQ | DY(SEQ | VTVSS |
| | RLSCAVS | (SEQ | VG | KS(SEQ | MNSLRAED | ID NO: | (SEQ ID |
| | (SEQ ID | ID NO: | (SEQ | ID NO: | TAVYYCAR | 6009) | NO: |
| | NO: 8573) | 8574) | ID NO: | 6008) | (SEQ ID | | 6050) |
| | | | 6048) | | NO: 316) | | |
| 15H6- | QIQLVESGG | GFSINT | WIRQAP | YIYSSGT | RFTISRDT | GNWHYF | WGQGTL |
| HC_4 | GLVKPGGSL | GGYHWN | GKGLEW | TRYNPSL | AKNSFYLQ | DY(SEQ | VTVSS |
| | RLSCAVS | (SEQ | VG | KS(SEQ | MNSLRAED | ID NO: | (SEQ ID |
| | (SEQ ID | ID NO: | (SEQ | ID NO: | TAVYYCAR | 6009) | NO: |
| | NO: 8575) | 8576) | ID NO: | 6008) | (SEQ ID | | 6054) |
| | | | 6052) | | NO: 6053) | | |
| 15H6- | QIQLVQSGA | GFSINT | WVRQAP | YIYSSGT | RVTMTRDT | GNWHYF | WGQGTL |
| HC_5 | EVKKPGASV | GGYHWN | GQGLEW | TRYNPSL | STNTFYME | DY(SEQ | VTVSS |
| | KVSCKVS | (SEQ | MG | KS(SEQ | LSSLRSED | ID NO: | (SEQ ID |
| | (SEQ ID | ID NO: | (SEQ | ID NO: | TAVYYCAR | 6009) | NO: |
| | NO: 8577) | 8578) | ID NO: | 6008) | (SEQ ID | | 6058) |
| | | | 6056) | | NO: 6057) | | |
| 15H6- | EIQLVQSGA | GFSINT | WVQQAP | YIYSSGT | RVTITRDT | GNWHYF | WGQGTL |
| HC_6 | EVKKPGATV | GGYHWN | GKGLEW | TRYNPSL | STNTFYME | DY(SEQ | VTVSS |
| | KISCKVS | (SEQ | MG | KS(SEQ | LSSLRSED | ID NO: | (SEQ ID |
| | (SEQ ID | ID NO: | (SEQ | ID NO: | TAVYYCAR | 6009) | NO: |
| | NO: 8579) | 8580) | ID NO: | 6008) | (SEQ ID | | 6062) |
| | | | 6060) | | NO: 6061) | | |

TABLE 21A
Exemplary heavy chain CDRs and FWRs of NKp30-targeting antigen
binding domains (according to the Kabat numbering scheme)

| Ab ID | VHFWR1 | VHCDR1 | VHFWR2 | VHCDR2 | VHFWR3 | VHCDR3 | VHFWR4 |
|---|---|---|---|---|---|---|---|
| 9G1- | QIQLQESGP | GYHWN | WIRQFPG | YIYSSGS | RISITRDT | GNWHYF | WGQGTM |
| HC | GLVKPSQSL | (SEQ | KKLEWMG | TSYNPSL | SKNQFFLQ | DF(SEQ | VTVSS |
| | SLTCSVTGF | ID NO: | (SEQ ID | KS | LNSVTTED | ID NO: | (SEQ ID |
| | SINTG | 7313) | NO: | (SEQ ID | ATTYYCAR | 6002) | NO: 304) |
| | (SEQ ID | | 6004) | NO: | (SEQ ID | | |
| | NO: 7317) | | | 6001) | NO: 6005) | | |
| 15H6- | QIQLQESGP | GYHWN | WIRQFPG | YIYSSGT | RISITRDT | GNWHYF | WGQGTL |
| HC | GLVKPSQSL | (SEQ | KKLEWMG | TRYNPSL | SKNQFFLQ | DY(SEQ | VAVSS |
| | SLTCSVTGF | ID NO: | (SEQ ID | KS | LNSVTPED | ID NO: | (SEQ ID |
| | SINTG | 7313) | NO: | (SEQ ID | TATYYCTR | 6009) | NO: |
| | (SEQ ID | | 6011) | NO: | (SEQ ID | | 6013) |
| | NO: 7317) | | | 6008) | NO: 6012) | | |
| 9G1- | QIQLQESGP | GYHWN | WIRQPAG | YIYSSGS | RVTMSRDT | GNWHYF | WGQGTM |
| HC_1 | GLVKPSETL | (SEQ | KGLEWIG | TSYNPSL | SKNQFSLK | DF(SEQ | VTVSS |
| | SLTCTVSGF | ID NO: | (SEQ ID | KS | LSSVTAAD | ID NO: | (SEQ ID |
| | SINTG | 7313) | NO: | (SEQ ID | TAVYYCAR | 6002) | NO: 304) |
| | (SEQ ID | | 6015) | NO: | (SEQ ID | | |
| | NO: 7371) | | | 6001) | NO: 6016) | | |
| 9G1- | QIQLQESGP | GYHWN | WIRQHPG | YIYSSGS | LVTISRDT | GNWHYF | WGQGTM |
| HC_2 | GLVKPSQTL | (SEQ | KGLEWIG | TSYNPSL | SKNQFSLK | DF(SEQ | VTVSS |
| | SLTCTVSGF | ID NO: | (SEQ ID | KS | LSSVTAAD | ID NO: | (SEQ ID |
| | SINTG | 7313) | NO: | (SEQ ID | ATVYYCAR | 6002) | NO: 304) |
| | (SEQ ID | | 6019) | NO: | (SEQ ID | | |
| | NO: 7372) | | | 6001) | NO: 6020) | | |
| 9G1- | EIQLLESGG | GYHWN | WVRQAPG | YIYSSGS | RFTISRDT | GNWHYF | WGQGTM |
| HC_3 | GLVQPGGSL | (SEQ | KGLEWVG | TSYNPSL | SKNTFYLQ | DF(SEQ | VTVSS |
| | RLSCAVSGF | ID NO: | (SEQ ID | KS | MNSLRAED | ID NO: | (SEQ ID |
| | SINTG | 7313) | NO: | (SEQ ID | TAVYYCAR | 6002) | NO: 304) |
| | (SEQ ID | | 6023) | NO: | (SEQ ID | | |
| | NO: 7373) | | | 6001) | NO: 316) | | |
| 9G1- | QIQLVQSGA | GYHWN | WVRQAPG | YIYSSGS | RVTITRDT | GNWHYF | WGQGTM |
| HC_4 | EVKKPGSSV | (SEQ | QGLEWMG | TSYNPSL | STNTFYME | DF(SEQ | VTVSS |
| | KVSCKVSGF | ID NO: | (SEQ ID | KS | LSSLRSED | ID NO: | (SEQ ID |
| | SINTG | 7313) | NO: | (SEQ ID | TAVYYCAR | 6002) | NO: 304) |
| | (SEQ ID | | 6027) | NO: | (SEQ ID | | |
| | NO: 7374) | | | 6001) | NO: 6028) | | |
| 9G1- | EIQLVESGG | GYHWN | WVRQAPG | YIYSSGS | RFTISRDT | GNWHYF | WGQGTM |
| HC_5 | GLVQPGGSL | (SEQ | KGLEWVG | TSYNPSL | AKNSFYLQ | DF(SEQ | VTVSS |
| | RLSCAVSGF | ID NO: | (SEQ ID | KS | MNSLRAED | ID NO: | (SEQ ID |
| | SINTG | 7313) | NO: | (SEQ ID | TAVYYCAR | 6002) | NO: 304) |
| | (SEQ ID | | 6032) | NO: | (SEQ ID | | |
| | NO: 7375) | | | 6001) | NO: 6033) | | |
| 9G1- | QIQLVQSGA | GYHWN | WVRQAPG | YIYSSGS | RVTMTRDT | GNWHYF | WGQGTM |
| HC_6 | EVKKPGASV | (SEQ | QGLEWMG | TSYNPSL | STNTFYME | DF(SEQ | VTVSS |
| | KVSCKVSGF | ID NO: | (SEQ ID | KS | LSSLRSED | ID NO: | (SEQ ID |
| | SINTG | 7313) | NO: | (SEQ ID | TAVYYCAR | 6002) | NO: 304) |
| | (SEQ ID | | 6036) | NO: | (SEQ ID | | |
| | NO: 7376) | | | 6001) | NO: 6037) | | |
| 15H6- | QIQLQESGP | GYHWN | WIRQHPG | YIYSSGT | LVTISRDT | GNWHYF | WGQGTL |
| HC_1 | GLVKPSQTL | (SEQ | KGLEWIG | TRYNPSL | SKNQFSLK | DY(SEQ | VTVSS |
| | SLTCTVSGF | ID NO: | (SEQ ID | KS | LSSVTAAD | ID NO: | (SEQ ID |
| | SINTG | 7313) | NO: | (SEQ ID | TAVYYCAR | 6009) | NO: |
| | (SEQ ID | | 6040) | NO: | (SEQ ID | | 6042) |
| | NO: 7372) | | | 6008) | NO: 6041) | | |
| 15H6- | QIQLQESGP | GYHWN | WIRQPAG | YIYSSGT | RVTMSRDT | GNWHYF | WGQGTL |
| HC_2 | GLVKPSETL | (SEQ | KGLEWIG | TRYNPSL | SKNQFSLK | DY(SEQ | VTVSS |
| | SLTCTVSGF | ID NO: | (SEQ ID | KS | LSSVTAAD | ID NO: | (SEQ ID |
| | SINTG | 7313) | NO: | (SEQ ID | TAVYYCAR | 6009) | NO: |
| | (SEQ ID | | 6044) | NO: | (SEQ ID | | 6046) |
| | NO: 7371) | | | 6008) | NO: 6045) | | |
| 15H6- | EIQLLESGG | GYHWN | WVRQAPG | YIYSSGT | RFTISRDT | GNWHYF | WGQGTL |
| HC_3 | GLVQPGGSL | (SEQ | KGLEWVG | TRYNPSL | SKNTFYLQ | DY(SEQ | VTVSS |
| | RLSCAVSGF | ID NO: | (SEQ ID | KS | MNSLRAED | ID NO: | (SEQ ID |
| | SINTG | 7313) | NO: | (SEQ ID | TAVYYCAR | 6009) | NO: |
| | (SEQ ID | | 6048) | NO: | (SEQ ID | | 6050) |
| | NO: 7373) | | | 6008) | NO: 316) | | |
| 15H6- | QIQLVESGG | GYHWN | WIRQAPG | YIYSSGT | RFTISRDT | GNWHYF | WGQGTL |
| HC_4 | GLVKPGGSL | (SEQ | KGLEWVG | TRYNPSL | AKNSFYLQ | DY(SEQ | VTVSS |
| | RLSCAVSGF | ID NO: | (SEQ ID | KS | MNSLRAED | ID NO: | (SEQ ID |
| | SINTG | 7313) | NO: | (SEQ ID | TAVYYCAR | 6009) | NO: |
| | (SEQ ID | | 6052) | NO: | (SEQ ID | | 6054) |
| | NO: 7377) | | | 6008) | NO: 6053) | | |
| 15H6- | QIQLVQSGA | GYHWN | WVRQAPG | YIYSSGT | RVTMTRDT | GNWHYF | WGQGTL |
| HC_5 | EVKKPGASV | (SEQ | QGLEWMG | TRYNPSL | STNTFYME | DY(SEQ | VTVSS |
| | KVSCKVSGF | ID NO: | (SEQ ID | KS | LSSLRSED | ID NO: | (SEQ ID |
| | SINTG | 7313) | NO: | (SEQ ID | TAVYYCAR | 6009) | NO: |
| | (SEQ ID | | 6056) | NO: | (SEQ ID | | 6058) |
| | NO: 7376) | | | 6008) | NO: 6057) | | |
| 15H6- | EIQLVQSGA | GYHWN | WVQQAPG | YIYSSGT | RVTITRDT | GNWHYF | WGQGTL |
| HC_6 | EVKKPGATV | (SEQ | KGLEWMG | TRYNPSL | STNTFYME | DY(SEQ | VTVSS |
| | KISCKVSGF | ID NO: | (SEQ ID | KS | LSSLRSED | ID NO: | (SEQ ID |
| | SINTG | 7313) | NO: | (SEQ ID | TAVYYCAR | 6009) | NO: |
| | (SEQ ID | | 6060) | NO: | (SEQ ID | | 6062) |
| | NO: 7378) | | | 6008) | NO: 6061) | | |
| 9D9- | QIQLQESGP | GYHWN | WIRQFPG | YIYSSGT | RISITRDT | GDWHYF | WGQGTM |
| HC | GLVKPSQSL | (SEQ | KKVEWMG | TKYNPSL | SKNQFFLQ | DY(SEQ | VAVSS |
| | SLSCSVTGF | ID NO: | (SEQ ID | KS | LNSVTTED | ID NO: | (SEQ ID |
| | SINTG | 7313) | NO: | (SEQ ID | TATYYCAR | 504) | NO: |
| | (SEQ ID | | 7314) | NO: | (SEQ ID | | 7316) |
| | NO: 7312) | | | 7385) | NO: 6005) | | |
| 3A12- | QIQLQESGP | GYHWN | WIRQFPG | YIYSSGS | RFSITRDT | GNWHYF | WGQGTL |
| HC | GLVKPSQSL | (SEQ | KKLEWMG | TRYNPSL | SKNQFFLQ | DY(SEQ | VAVSS |
| | SLTCSVTGF | ID NO: | (SEQ ID | KS | LNSVTTED | ID NO: | (SEQ ID |
| | SINTG | 7313) | NO: | (SEQ ID | TATYYCTR | 6009) | NO: |
| | (SEQ ID | | 6004) | NO: | (SEQ ID | | 6013) |
| | NO: 7317) | | | 7318) | NO: 7319) | | |
| 12D10- | QIQLQESGP | GYHWN | WIRQFPG | YIYSSGT | RISITRDT | GNWHYF | WGQGTL |
| HC | GLVKPSQSL | (SEQ | KKLEWMG | TRYNPSL | SKNQFFLQ | DY(SEQ | VAVSS |
| | SLTCSVTGF | ID NO: | (SEQ ID | KS | LNSVTPED | ID NO: | (SEQ ID |
| | SINTG | 7313) | NO: | (SEQ ID | TATYYCTR | 6009) | NO: |
| | (SEQ ID | | 6004) | NO: | (SEQ ID | | 6013) |
| | NO: 7317) | | | 6008) | NO: 6012) | | |
| 15E1- | QIQLQESGP | GYHWN | WIRQFPG | YIYSSGS | RFSITRDT | GDWHYF | WGPGTM |
| HC | GLVKPSQSL | (SEQ | KKLEWMG | TSYNPSL | SKNQFFLQ | DY(SEQ | VTVSS |
| | SLSCSVTGF | ID NO: | (SEQ ID | KS | LNSVTTED | ID NO: | (SEQ ID |
| | SITTT | 7313) | NO: | (SEQ ID | TATYYCAR | 504) | NO: |
| | (SEQ ID | | 6004) | NO: | (SEQ ID | | 7324) |
| | NO: 7322) | | | 6001) | NO: 7323) | | |
| 15E1_ | QIQLQESGP | GYHWN | WIRQHPG | YIYSSGS | LVTISRDT | GDWHYF | WGQGTM |
| Human- | GLVKPSQTL | (SEQ | KGLEWIG | TSYNPSL | SKNQFSLK | DY(SEQ | VTVSS |
| ized | SLTCTVSGF | ID NO: | (SEQ ID | KS | LSSVTAAD | ID NO: | (SEQ ID |
| variant_ | SITTT | 7313) | NO: | (SEQ ID | TAVYYCAR | 504) | NO: 304) |
| VH1 | (SEQ ID | | 6019) | NO: | (SEQ ID | | |
| | NO: 7330) | | | 6001) | NO: 6020) | | |
| 15E1_ | QIQLVESGG | GYHWN | WIRQAPG | YIYSSGS | RFTISRDT | GDWHYF | WGQGTM |
| Human- | GLVKPGGSL | (SEQ | KGLEWVG | TSYNPSL | AKNSFYLQ | DY(SEQ | VTVSS |
| ized | RLSCAVSG | ID NO: | (SEQ ID | KS | MNSLRAED | ID NO: | (SEQ ID |
| variant_ | FSITTT | 7313) | NO: | (SEQ ID | TAVYYCAR | 504) | NO: 304) |
| VH2 | (SEQ ID | | 6052) | NO: | (SEQ ID | | |
| | NO: 7331) | | | 6001) | NO: 6033) | | |
| 15E1_ | EIQLLESGG | GYHWN | WVRQAPG | YIYSSGS | RFTISRDT | GDWHYF | WGQGTM |
| Human- | GLVQPGGSL | (SEQ | KGLEWVG | TSYNPSL | SKNTFYLQ | DY(SEQ | VTVSS |
| ized | RLSCAVSGF | ID NO: | (SEQ ID | KS | MNSLRAED | ID NO: | (SEQ ID |
| variant_ | SITTT | 7313) | NO: | (SEQ ID | TAVYYCAR | 504) | NO: 304) |
| VH3 | (SEQ ID | | 6023) | NO: | (SEQ ID | | |
| | NO: 7332) | | | 6001) | NO: 316) | | |
| 15E1_ | EIQLVESGG | GYHWN | WVRQAPG | YIYSSGS | RFTISRDT | GDWHYF | WGQGTM |
| Human- | GLVQPGGSL | (SEQ | KGLEWVG | TSYNPSL | AKNSFYLQ | DY(SEQ | VTVSS |
| ized | RLSCAVSGF | ID NO: | (SEQ ID | KS | MNSLRAED | ID NO: | (SEQ ID |
| variant_ | SITTT | 7313) | NO: | (SEQ ID | TAVYYCAR | 504) | NO: 304) |
| VH4 | (SEQ ID | | 6023) | NO: | (SEQ ID | | |
| | NO: 7333) | | | 6001) | NO: 6033) | | |
| 15E1_ | QIQLVQSGA | GYHWN | WVRQAPG | YIYSSGS | RVTMTRDT | GDWHYF | WGQGTM |
| Human- | EVKKPGASV | (SEQ | QGLEWMG | TSYNPSL | STNTFYME | DY(SEQ | VTVSS |
| ized | KVSCKVSGF | ID NO: | (SEQ ID | KS | LSSLRSED | ID NO: | (SEQ ID |
| variant_ | SITTT | 7313) | NO: | (SEQ ID | TAVYYCAR | 504) | NO: 304) |
| VH5 | (SEQ ID | | 6027) | NO: | (SEQ ID | | |
| | NO: 7334) | | | 6001) | NO: 6037) | | |

TABLE 21B
Exemplary heavy chain CDRs and FWRs of NKp30-targeting
antigen binding domains of Table 21A (According to the
ABM numbering scheme)

| Ab ID | VHFWR1 | VHCDR1 | VHFWR2 | VHCDR2 | VHFWR3 | VHCDR3 | VHFWR4 |
|---|---|---|---|---|---|---|---|
| 9G1- | QIQLQESGP | GFSINT | WIRQFPG | YIYSSGS | RISITRDTS | GNWHYF | WGQGTM |
| HC | GLVKPSQSL | GGYHWN | KKLEWMG | TSYNPSL | KNQFFLQLN | DF | VTVSS |
| | SLTCSVT | (SEQ | (SEQ ID | KS | SVTTEDTAT | (SEQ | (SEQ ID |
| | (SEQ ID | ID NO: | NO: | (SEQ ID | YYCAR | ID NO: | NO: 304) |
| | NO: 8581) | 8582) | 6004) | NO: | (SEQ ID | 6002) | |
| | | | | 6001) | NO: 6005) | | |
| 15H6- | QIQLQESGP | GFSINT | WIRQFPG | YIYSSGT | RISITRDTS | GNWHYF | WGQGTL |
| HC | GLVKPSQSL | GGYHWN | KKLEWMG | TRYNPSL | KNQFFLQLN | DY | VAVSS |
| | SLTCSVT | (SEQ | (SEQ ID | KS | SVTPEDTAT | (SEQ | (SEQ ID |
| | (SEQ ID | ID NO: | NO: | (SEQ ID | YYCTR | ID NO: | NO: |
| | NO: 8583) | 8584) | 6011) | NO: | (SEQ ID | 6009) | 6013) |
| | | | | 6008) | NO: 6012) | | |
| 9G1- | QIQLQESGP | GFSINT | WIRQPAG | YIYSSGS | RVTMSRDTS | GNWHYF | WGQGTM |
| HC_1 | GLVKPSETL | GGYHWN | KGLEWIG | TSYNPSL | KNQFSLKLS | DF | VTVSS |
| | SLTCTVS | (SEQ | (SEQ ID | KS | SVTAADTAV | (SEQ | (SEQ ID |
| | (SEQ ID | ID NO: | NO: | (SEQ ID | YYCAR | ID NO: | NO: 304) |
| | NO: 8585) | 8586) | 6015) | NO: | (SEQ ID | 6002) | |
| | | | | 6001) | NO: 6016) | | |
| 9G1- | QIQLQESGP | GFSINT | WIRQHPG | YIYSSGS | LVTISRDTS | GNWHYF | WGQGTM |
| HC_2 | GLVKPSQTL | GGYHWN | KGLEWIG | TSYNPSL | KNQFSLKLS | DF | VTVSS |
| | SLTCTVS | (SEQ | (SEQ ID | KS | SVTAADTAV | (SEQ | (SEQ ID |
| | (SEQ ID | ID NO: | NO: | (SEQ ID | YYCAR | ID NO: | NO: 304) |
| | NO: 8587) | 8588) | 6019) | NO: | (SEQ ID | 6002) | |
| | | | | 6001) | NO: 6020) | | |
| 9G1- | EIQLLESGG | GFSINT | WVRQAPG | YIYSSGS | RFTISRDTS | GNWHYF | WGQGTM |
| HC_3 | GLVQPGGSL | GGYHWN | KGLEWVG | TSYNPSL | KNTFYLQMN | DF | VTVSS |
| | RLSCAVS | (SEQ | (SEQ ID | KS | SLRAEDTAV | (SEQ | (SEQ ID |
| | (SEQ ID | ID NO: | NO: | (SEQ ID | YYCAR | ID NO: | NO: 304) |
| | NO: 8589) | 8590) | 6023) | NO: | (SEQ ID | 6002) | |
| | | | | 6001) | NO: 316) | | |
| 9G1- | QIQLVQSGA | GFSINT | WVRQAPG | YIYSSGS | RVTITRDTS | GNWHYF | WGQGTM |
| HC_4 | EVKKPGSSV | GGYHWN | QGLEWMG | TSYNPSL | TNTFYMELS | DF | VTVSS |
| | KVSCKVS | (SEQ | (SEQ ID | KS | SLRSEDTAV | (SEQ | (SEQ ID |
| | (SEQ ID | ID NO: | NO: | (SEQ ID | YYCAR | ID NO: | NO: 304) |
| | NO: 8591) | 8592) | 6027) | NO: | (SEQ ID | 6002) | |
| | | | | 6001) | NO: 6028) | | |
| 9G1- | EIQLVESGG | GFSINT | WVRQAPG | YIYSSGS | RFTISRDTA | GNWHYF | WGQGTM |
| HC_5 | GLVQPGGSL | GGYHWN | KGLEWVG | TSYNPSL | KNSFYLQMN | DF | VTVSS |
| | RLSCAVS | (SEQ | (SEQ ID | KS | SLRAEDTAV | (SEQ | (SEQ ID |
| | (SEQ ID | ID NO: | NO: | (SEQ ID | YYCAR | ID NO: | NO: 304) |
| | NO: 8593) | 8594) | 6032) | NO: | (SEQ ID | 6002) | |
| | | | | 6001) | NO: 6033) | | |
| 9G1- | QIQLVQSGA | GFSINT | WVRQAPG | YIYSSGS | RVTMTRDTS | GNWHYF | WGQGTM |
| HC_6 | EVKKPGASV | GGYHWN | QGLEWMG | TSYNPSL | TNTFYMELS | DF | VTVSS |
| | KVSCKVS | (SEQ | (SEQ ID | KS | SLRSEDTAV | (SEQ | (SEQ ID |
| | (SEQ ID | ID NO: | NO: | (SEQ ID | YYCAR | ID NO: | NO: 304) |
| | NO: 8595) | 8596) | 6036) | NO: | (SEQ ID | 6002) | |
| | | | | 6001) | NO: 6037) | | |
| 15H6- | QIQLQESGP | GFSINT | WIRQHPG | YIYSSGT | LVTISRDTS | GNWHYF | WGQGTL |
| HC_1 | GLVKPSQTL | GGYHWN | KGLEWIG | TRYNPSL | KNQFSLKLS | DY | VTVSS |
| | SLTCTVS | (SEQ | (SEQ ID | KS | SVTAADTAV | (SEQ | (SEQ ID |
| | (SEQ ID | ID NO: | NO: | (SEQ ID | YYCAR | ID NO: | NO: |
| | NO: 8597) | 8598) | 6040) | NO: | (SEQ ID | 6009) | 6042) |
| | | | | 6008) | NO: 6041) | | |
| 15H6- | QIQLQESGP | GFSINT | WIRQPAG | YIYSSGT | RVTMSRDTS | GNWHYF | WGQGTL |
| HC_2 | GLVKPSETL | GGYHWN | KGLEWIG | TRYNPSL | KNQFSLKLS | DY | VTVSS |
| | SLTCTVS | (SEQ | (SEQ ID | KS | SVTAADTAV | (SEQ | (SEQ ID |
| | (SEQ ID | ID NO: | NO: | (SEQ ID | YYCAR | ID NO: | NO: |
| | NO: 8599) | 8600) | 6044) | NO: | (SEQ ID | 6009) | 6046) |
| | | | | 6008) | NO: 6045) | | |
| 15H6- | EIQLLESGG | GFSINT | WVRQAPG | YIYSSGT | RFTISRDTS | GNWHYF | WGQGTL |
| HC_3 | GLVQPGGSL | GGYHWN | KGLEWVG | TRYNPSL | KNTFYLQMN | DY | VTVSS |
| | RLSCAVS | (SEQ | (SEQ ID | KS | SLRAEDTAV | (SEQ | (SEQ ID |
| | (SEQ ID | ID NO: | NO: | (SEQ ID | YYCAR | ID NO: | NO: |
| | NO: 8601) | 8602) | 6048) | NO: | (SEQ ID | 6009) | 6050) |
| | | | | 6008) | NO: 316) | | |
| 15H6- | QIQLVESGG | GFSINT | WIRQAPG | YIYSSGT | RFTISRDTA | GNWHYF | WGQGTL |
| HC_4 | GLVKPGGSL | GGYHWN | KGLEWVG | TRYNPSL | KNSFYLQMN | DY | VTVSS |
| | RLSCAVS | (SEQ | (SEQ ID | KS | SLRAEDTAV | (SEQ | (SEQ ID |
| | (SEQ ID | ID NO: | NO: | (SEQ ID | YYCAR | ID NO: | NO: |
| | NO: 8603) | 8604) | 6052) | NO: | (SEQ ID | 6009) | 6054) |
| | | | | 6008) | NO: 6053) | | |
| 15H6- | QIQLVQSGA | GFSINT | WVRQAPG | YIYSSGT | RVTMTRDTS | GNWHYF | WGQGTL |
| HC_5 | EVKKPGASV | GGYHWN | QGLEWMG | TRYNPSL | TNTFYMELS | DY | VTVSS |
| | KVSCKVS | (SEQ | (SEQ ID | KS | SLRSEDTAV | (SEQ | (SEQ ID |
| | (SEQ ID | ID NO: | NO: | (SEQ ID | YYCAR | ID NO: | NO: |
| | NO: 8605) | 8606) | 6056) | NO: | (SEQ ID | 6009) | 6058) |
| | | | | 6008) | NO: 6057) | | |
| 15H6- | EIQLVQSGA | GFSINT | WVQQAPG | YIYSSGT | RVTITRDTS | GNWHYF | WGQGTL |
| HC_6 | EVKKPGATV | GGYHWN | KGLEWMG | TRYNPSL | TNTFYMELS | DY | VTVSS |
| | KISCKVS | (SEQ | (SEQ ID | KS | SLRSEDTAV | (SEQ | (SEQ ID |
| | (SEQ ID | ID NO: | NO: | (SEQ ID | YYCAR | ID NO: | NO: |
| | NO: 8607) | 8608) | 6060) | NO: | (SEQ ID | 6009) | 6062) |
| | | | | 6008) | NO: 6061) | | |
| 9D9- | QIQLQESGP | GFSINT | WIRQFPG | YIYSSGT | RISITRDTS | GDWHYF | WGQGTM |
| HC | GLVKPSQSL | GGYHWN | KKVEWMG | TKYNPSL | KNQFFLQLN | DY | VAVSS |
| | SLSCSVT | (SEQ | (SEQ ID | KS | SVTTEDTAT | (SEQ | (SEQ ID |
| | (SEQ ID | ID NO: | NO: | (SEQ ID | YYCAR | ID NO: | NO: |
| | NO: 8609) | 8610) | 7314) | NO: | (SEQ ID | 504) | 7316) |
| | | | | 7385) | NO: 6005) | | |
| 3A12- | QIQLQESGP | GFSINT | WIRQFPG | YIYSSGS | RFSITRDTS | GNWHYF | WGQGTL |
| HC | GLVKPSQSL | GGYHWN | KKLEWMG | TRYNPSL | KNQFFLQLN | DY | VAVSS |
| | SLTCSVT | (SEQ | (SEQ ID | KS | SVTTEDTAT | (SEQ | (SEQ ID |
| | (SEQ ID | ID NO: | NO: | (SEQ ID | YYCTR | ID NO: | NO: |
| | NO: 8611) | 8612) | 6004) | NO: | (SEQ ID | 6009) | 6013) |
| | | | | 7318) | NO: 7319) | | |
| 12D10- | QIQLQESGP | GFSINT | WIRQFPG | YIYSSGT | RISITRDTS | GNWHYF | WGQGTL |
| HC | GLVKPSQSL | GGYHWN | KKLEWMG | TRYNPSL | KNQFFLQLN | DY | VAVSS |
| | SLTCSVT | (SEQ | (SEQ ID | KS | SVTPEDTAT | (SEQ | (SEQ ID |
| | (SEQ ID | ID NO: | NO: | (SEQ ID | YYCTR | ID NO: | NO: |
| | NO: 8613) | 8614) | 6004) | NO: | (SEQ ID | 6009) | 6013) |
| | | | | 6008) | NO: 6012) | | |
| 15E1- | QIQLQESGP | GFSITT | WIRQFPG | YIYSSGS | RFSITRDTS | GDWHYF | WGPGTM |
| HC | GLVKPSQSL | TGYHWN | KKLEWMG | TSYNPSL | KNQFFLQLN | DY | VTVSS |
| | SLSCSVT | (SEQ | (SEQ ID | KS | SVTTEDTAT | (SEQ | (SEQ ID |
| | (SEQ ID | ID NO: | NO: | (SEQ ID | YYCAR | ID NO: | NO: |
| | NO: 8615) | 8616) | 6004) | NO: | (SEQ ID | 504) | 7324) |
| | | | | 6001) | NO: 7323) | | |
| 15E1_ | QIQLQESGP | GFSITT | WIRQHPG | YIYSSGS | LVTISRDTS | GDWHYF | WGQGTM |
| Human- | GLVKPSQTL | TGYHWN | KGLEWIG | TSYNPSL | KNQFSLKLS | DY | VTVSS |
| ized | SLTCTVS | (SEQ | (SEQ ID | KS | SVTAADTAV | (SEQ | (SEQ ID |
| variant_ | (SEQ ID | ID NO: | NO: | (SEQ ID | YYCAR | ID NO: | NO: 304) |
| VH1 | NO: 8617) | 8618) | 6019) | NO: | (SEQ ID | 504) | |
| | | | | 6001) | NO: 6020) | | |
| 15E1_ | QIQLVESGG | GFSITT | WIRQAPG | YIYSSGS | RFTISRDTA | GDWHYF | WGQGTM |
| Human- | GLVKPGGSL | TGYHWN | KGLEWVG | TSYNPSL | KNSFYLQMN | DY | VTVSS |
| ized | RLSCAVS | (SEQ | (SEQ ID | KS | SLRAEDTAV | (SEQ | (SEQ ID |
| variant_ | (SEQ ID | ID NO: | NO: | (SEQ ID | YYCAR | ID NO: | NO: 304) |
| VH2 | NO: 8619) | 8620) | 6052) | NO: | (SEQ ID | 504) | |
| | | | | 6001) | NO: 6033) | | |
| 15E1_ | EIQLLESGG | GFSITT | WVRQAPG | YIYSSGS | RFTISRDTS | GDWHYF | WGQGTM |
| Human- | GLVQPGGSL | TGYHWN | KGLEWVG | TSYNPSL | KNTFYLQMN | DY | VTVSS |
| ized | RLSCAVS | (SEQ | (SEQ ID | KS | SLRAEDTAV | (SEQ | (SEQ ID |
| variant_ | (SEQ ID | ID NO: | NO: | (SEQ ID | YYCAR | ID NO: | NO: 304) |
| VH3 | NO: 8621) | 8622) | 6023) | NO: | (SEQ ID | 504) | |
| | | | | 6001) | NO: 316) | | |
| 15E1_ | EIQLVESGG | GFSITT | WVRQAPG | YIYSSGS | RFTISRDTA | GDWHYF | WGQGTM |
| Human- | GLVQPGGSL | TGYHWN | KGLEWVG | TSYNPSL | KNSFYLQMN | DY | VTVSS |
| ized | RLSCAVS | (SEQ | (SEQ ID | KS | SLRAEDTAV | (SEQ | (SEQ ID |
| variant_ | (SEQ ID | ID NO: | NO: | (SEQ ID | YYCAR | ID NO: | NO: 304) |
| VH4 | NO: 8623) | 8624) | 6023) | NO: | (SEQ ID | 504) | |
| | | | | 6001) | NO: 6033) | | |
| 15E1_ | QIQLVQSGA | GFSITT | WVRQAPG | YIYSSGS | RVTMTRDTS | GDWHYF | WGQGTM |
| Human- | EVKKPGASV | TGYHWN | QGLEWMG | TSYNPSL | TNTFYMELS | DY | VTVSS |
| ized | KVSCKVS | (SEQ | (SEQ ID | KS | SLRSEDTAV | (SEQ | (SEQ ID |
| variant_ | (SEQ ID | ID NO: | NO: | (SEQ ID | YYCAR | ID NO: | NO: 304) |
| VH5 | NO: 8625) | 8626) | 6027) | NO: | (SEQ ID | 504) | |
| | | | | 6001) | NO: 6037) | | |

TABLE 22
Exemplary light chain CDRs and FWRs of NKp30-targeting
antigen binding domains

| Ab ID | VLFWR1 | VLCDR1 | VLFWR2 | VLCDR2 | VLFWR3 | VLCDR3 | VLFWR4 |
|---|---|---|---|---|---|---|---|
| 9G1- | SYTLTQPPL | SGERLS | WYQQKP | ENDKRP | GIPDQFSG | QSWDST | FGSGTQL |
| LC | LSVALGHKA | DKYVH | GRAPVM | S | SNSGNIAT | NSAV | TVL |
| | TITC | (SEQ | VIY | (SEQ | LTISKAQA | (SEQ | (SEQ |
| | (SEQ | ID NO: | (SEQ | ID NO: | GYEADYYC | ID NO: | ID NO: |
| | ID NO: | 6063) | ID NO: | 6064) | (SEQ ID | 7293) | 6069) |
| | 6066) | | 6067) | | NO: 7292) | | |
| 15H6- | SYTLTQPPS | SGENLS | WYQQKP | ENEKRP | GIPDQFSG | HYWESI | FGSGTHL |
| LC | LSVAPGQKA | DKYVH | GRAPVM | S | SNSGNIAT | NSVV | TVL |
| | TIIC | (SEQ | VIY | (SEQ | LTISKAQP | (SEQ | (SEQ |
| | (SEQ | ID NO: | (SEQ | ID NO: | GSEADYYC | ID NO: | ID NO: |
| | ID NO: | 6070) | ID NO: | 6071) | (SEQ ID | 6072) | 6076) |
| | 6073) | | 6074) | | NO: 6075) | | |
| 9G1- | QSVTTQPPS | SGERLS | WYQQLP | ENDKRP | GVPDRFSG | QSWDST | FGGGTQL |
| LC_1 | VSGAPGQRV | DKYVH | GTAPKM | S | SNSGNSAS | NSAV | TVL |
| | TISC | (SEQ | LIY | (SEQ | LAITGLQA | (SEQ | (SEQ |
| | (SEQ | ID NO: | (SEQ | ID NO: | EDEADYYC | ID NO: | ID NO: |
| | ID NO: | 6063) | ID NO: | 6064) | (SEQ ID | 7293) | 6080) |
| | 6077) | | 6078) | | NO: 6079) | | |
| 9G1- | QSVTTQPPS | SGERLS | WYQQLP | ENDKRP | GVPDRFSG | QSWDST | FGGGTQL |
| LC_2 | ASGTPGQRV | DKYVH | GTAPKM | S | SNSGNSAS | NSAV | TVL |
| | TISC | (SEQ | LIY | (SEQ | LAISGLQS | (SEQ | (SEQ |
| | (SEQ | ID NO: | (SEQ | ID NO: | EDEADYYC | ID NO: | ID NO: |
| | ID NO: | 6063) | ID NO: | 6064) | (SEQ ID | 7293) | 6080) |
| | 6081) | | 6082) | | NO: 6083) | | |
| 9G1- | QSVTTQPPS | SGERLS | WYQQLP | ENDKRP | GVPDRFSG | QSWDST | FGGGTQL |
| LC_3 | ASGTPGQRV | DKYVH | GTAPKM | S | SNSGNSAS | NSAV | TVL |
| | TISC | (SEQ | LIY | (SEQ | LAISGLRS | (SEQ | (SEQ |
| | (SEQ | ID NO: | (SEQ | ID NO: | EDEADYYC | ID NO: | ID NO: |
| | ID NO: | 6063) | ID NO: | 6064) | (SEQ ID | 7293) | 6080) |
| | 6085) | | 6086) | | NO: 6087) | | |
| 9G1- | SSETTQPHS | SGERLS | WYQQKP | ENDKRP | GIPERFSG | QSWDST | FGGGTQL |
| LC_4 | VSVATAQM | DKYVH | GQDPVM | S | SNPGNTAT | NSAV | TVL |
| | ARITC | (SEQ | VIY | (SEQ | LTISRIEA | (SEQ | (SEQ |
| | (SEQ | ID NO: | (SEQ | ID NO: | GDEADYYC | ID NO: | ID NO: |
| | ID NO: | 6063) | ID NO: | 6064) | (SEQ ID | 7293) | 6080) |
| | 6089) | | 6090) | | NO: 6091) | | |
| 9G1- | DIQMTQSPS | SGERLS | WYQQKP | ENDKRP | GVPSRFSG | QSWDST | FGQGTKV |
| LC_5 | TLSASVGDR | DKYVH | GKAPKM | S | SNSGNEAT | NSAV | EIK |
| | VTITC | (SEQ | LIY | (SEQ | LTISSLQP | (SEQ | (SEQ |
| | (SEQ | ID NO: | (SEQ | ID NO: | DDFATYYC | ID NO: | ID NO: |
| | ID NO: | 6063) | ID NO: | 6064) | (SEQ ID | 7293) | 6096) |
| | 6093) | | 6094) | | NO: 6095) | | |
| 15H6- | QYVLTQPPS | SGENLS | WYQQLP | ENEKRP | GVPDRFSG | HYWESI | FGEGTEL |
| LC_1 | ASGTPGQRV | DKYVH | GTAPKM | S | SNSGNSAS | NSVV | TVL |
| | TISC | (SEQ | LIY | (SEQ | LAISGLQS | (SEQ | (SEQ |
| | (SEQ | ID NO: | (SEQ | ID NO: | EDEADYYC | ID NO: | ID NO: |
| | ID NO: | 6070) | ID NO: | 6071) | (SEQ ID | 6072) | 6100) |
| | 6097) | | 6098) | | NO: 6099) | | |
| 15H6- | QYVLTQPPS | SGENLS | WYQQLP | ENEKRP | GVPDRFSG | HYWESI | FGEGTEL |
| LC_2 | ASGTPGQRV | DKYVH | GTAPKM | S | SNSGNSAS | NSVV | TVL |
| | TISC | (SEQ | LIY | (SEQ | LAISGLRS | (SEQ | (SEQ |
| | (SEQ | ID NO: | (SEQ | ID NO: | EDEADYYC | ID NO: | ID NO: |
| | ID NO: | 6070) | ID NO: | 6071) | (SEQ ID | 6072) | 6104) |
| | 6101) | | 6102) | | NO: 6103) | | |
| 15H6- | SYELTQPPS | SGENLS | WYQQKP | ENEKRP | GIPERFSG | HYWESI | FGEGTEL |
| LC_3 | VSVSPGQTA | DKYVH | GQSPVM | S | SNSGNTAT | NSVV | TVL |
| | SITC | (SEQ | VIY | (SEQ | LTISGTQA | (SEQ | (SEQ |
| | (SEQ | ID NO: | (SEQ | ID NO: | MDEADYYC | ID NO: | ID NO: |
| | ID NO: | 6070) | ID NO: | 6071) | (SEQ ID | 6072) | 6108) |
| | 6105) | | 6106) | | NO: 6107) | | |
| 15H6- | DYVLTQSPL | SGENLS | WYLQKP | ENEKRP | GVPDRFSG | HYWESI | FGQGTKV |
| LC_4 | SLPVTPGEP | DKYVH | GQSPQM | S | SNSGNDAT | NSVV | EIK |
| | ASISC | (SEQ | LIY | (SEQ | LKISRVEA | (SEQ | (SEQ |
| | (SEQ | ID NO: | (SEQ | ID NO: | EDVGVYYC | ID NO: | ID NO: |
| | ID NO: | 6070) | ID NO: | 6071) | (SEQ ID | 6072) | 6112) |
| | 6109) | | 6110) | | NO: 6111) | | |
| 15H6- | AYQLTQSPS | SGENLS | WYQQKP | ENEKRP | GVPSRFSG | HYWESI | FGQGTKV |
| LC_5 | SLSASVGDR | DKYVH | GKAPKM | S | SNSGNDAT | NSVV | EIK |
| | VTITC | (SEQ | LIY | (SEQ | LTISSLQP | (SEQ | (SEQ |
| | (SEQ | ID NO: | (SEQ | ID NO: | EDFATYYC | ID NO: | ID NO: |
| | ID NO: | 6070) | ID NO: | 6071) | (SEQ ID | 6072) | 6116) |
| | 6113) | | 6114) | | NO: 6115) | | |
| 15H6- | EYVLTQSPA | SGENLS | WYQQKP | ENEKRP | GIPARFSG | HYWESI | FGQGTKV |
| LC_6 | TLSVSPGER | DKYVH | GQAPRM | S | SNSGNEAT | SVV | EIK |
| | ATLSC | (SEQ | LIY | (SEQ | LTISSLQS | (SEQ | (SEQ |
| | (SEQ | ID NO: | (SEQ | ID NO: | EDFAVYYC | ID NO: | ID NO: |
| | ID NO: | 6070) | ID NO: | 6071) | (SEQ ID | 6072) | 6120) |
| | 6117) | | 6118) | | NO: 6119) | | |
| 9D9- | SYTLTQPPL | SGENLS | WYQQKP | ENDKRP | GIPDQFSG | HCWDST | FGSGTHL |
| LC | VSVALGQKA | DKYVH | GRAPVM | S | SNSGNIAT | NSAV | TVL |
| | TIIC | (SEQ | VIY | (SEQ | LTISKAQA | (SEQ | (SEQ |
| | (SEQ | ID NO: | (SEQ | ID NO: | GYEADYYC | ID NO: | ID NO: |
| | ID NO: | 6070) | ID NO: | 6064) | (SEQ ID | 7321) | 6076) |
| | 7320) | | 6067) | | NO: 7292) | | |
| 3A12- | SYTLTQPPL | SGENLS | WYQQKP | ENDKRP | GIPDQFSG | HCWDST | FGSGTHL |
| LC | VSVALGQKA | DKYVH | GRAPVM | S | SNSGNIAT | NSAV | TVL |
| | TIIC | (SEQ | VIY | (SEQ | LTISKAQA | (SEQ | (SEQ |
| | (SEQ | ID NO: | (SEQ | ID NO: | GYEADYYC | ID NO: | ID NO: |
| | ID NO: | 6070) | ID NO: | 6064) | (SEQ ID | 7321) | 6076) |
| | 7320) | | 6067) | | NO: 7292) | | |
| 12D10- | SYTLTQPPS | SGENLS | WYQQKP | ENEKRP | GIPDQFSG | HYWESI | FGSGTHL |
| LC | LSVAPGQKA | DKYVH | GRAPVM | S | SNSGNIAT | NSVV | TVL |
| | TIIC | (SEQ | VIY | (SEQ | LTISKAQP | (SEQ | (SEQ |
| | (SEQ | ID NO: | (SEQ | ID NO: | GSEADYYC | ID NO: | ID NO: |
| | ID NO: | 6070) | ID NO: | 6071) | (SEQ ID | 6072) | 6076) |
| | 6073) | | 6074) | | NO: 6075) | | |
| 15E1- | SFTLTQPPL | SGEKLS | WYQQKP | ENDRRP | GIPDQFSG | QFWDST | FGGGTQL |
| LC | VSVAVGQVA | DKYVH | GRAPVM | S | SNSGNIAS | NSAV | TVL |
| | TITC | (SEQ | VIY | (SEQ | LTISKAQA | (SEQ | (SEQ |
| | (SEQ | ID NO: | (SEQ | ID NO: | GDEADYFC | ID NO: | ID NO: |
| | ID NO: | 26) | ID NO: | 28) | (SEQ ID | 86) | 6080) |
| | 7325) | | 6067) | | NO: 7328) | | |
| 15E1_ | SSETTQPPS | SGEKLS | WYQQKP | ENDRRP | GIPERFSG | QFWDST | FGGGTQL |
| Human- | VSVSPGQTA | DKYVH | GQSPVM | S | SNSGNTAT | NSAV | TVL |
| ized | SITC | (SEQ | VIY | (SEQ | LTISGTQA | (SEQ | (SEQ |
| variant_ | (SEQ | ID NO: | (SEQ | ID NO: | MDEADYFC | ID NO: | ID NO: |
| VL1 | ID NO: | 26) | ID NO: | 28) | (SEQ ID | 86) | 6080) |
| | 7335 | | 6106) | | NO: 7336) | | |
| 15E1_ | SSETTQPHS | SGEKLS | WYQQKP | ENDRRP | GIPERFSG | QFWDST | FGGGTQL |
| Human- | VSVATAQMA | DKYVH | GQDPVM | S | SNPGNTAT | NSAV | TVL |
| ized | RITC | (SEQ | VIY | (SEQ | LTISRIEA | (SEQ | (SEQ |
| variant_ | (SEQ | ID NO: | (SEQ | ID NO: | GDEADYFC | ID NO: | ID NO: |
| VL2 | ID NO: | 26) | ID NO: | 28) | (SEQ ID | 86) | 6080) |
| | 6089) | | 6090) | | NO: 7337) | | |
| 15E1_ | QSVTTQPPS | SGEKLS | WYQQLP | ENDRRP | GVPDRFSG | QFWDST | FGGGTQL |
| Human- | ASGTPGQRV | DKYVH | GTAPKM | S | SNSGNSAS | NSAV | TVL |
| ized | TISC | (SEQ | LIY | (SEQ | LAISGLRS | (SEQ | (SEQ |
| variant_ | (SEQ | ID NO: | (SEQ | ID NO: | EDEADYFC | ID NO: | ID NO: |
| VL3 | ID NO: | 26) | ID NO: | 28) | (SEQ ID | 86) | 6080) |
| | 6081) | | 6078) | | NO: 7338) | | |
| 15E1_ | QSVTTQPPS | SGEKLS | WYQQLP | ENDRRP | GVPDRFSG | QFWDST | FGGGTQL |
| Human- | VSGAPGQRV | DKYVH | GTAPKM | S | SNSGNSAS | NSAV | TVL |
| ized | TISC | (SEQ | LIY | (SEQ | LAITGLQA | (SEQ | (SEQ |
| variant_ | (SEQ | ID NO: | (SEQ | ID NO: | EDEADYFC | ID NO: | ID NO: |
| VL4 | ID NO: | 26) | ID NO: | 28) | (SEQ ID | 86) | 6080) |
| | 6077) | | 6078) | | NO: 7339) | | |
| 15E1_ | DSVTTQSPL | SGEKLS | WYQQRP | ENDRRP | GVPDRFSG | QFWDST | FGGGTKV |
| Human- | SLPVTLGQP | DKYVH | GQSPRM | S | SNSGNDAT | NSAV | EIK |
| ized | ASISC | (SEQ | LIY | (SEQ | LKISRVEA | (SEQ | (SEQ |
| variant_ | (SEQ | ID NO: | (SEQ | ID NO: | EDVGVYFC | ID NO: | ID NO: |
| VL5 | ID NO: | 26) | ID NO: | 28) | (SEQ ID | 86) | 8685) |
| | 7340) | | 7341) | | NO: 7342) | | |

TABLE 23A
Exemplary heavy chain CDRs and FWRs of NKp30-targeting
antigen binding domains

| Ab ID | VHFWR1 | VHCDR1 | VHFWR2 | VHCDR2 | VHFWR3 | VHCDR3 | VHFWR4 |
|---|---|---|---|---|---|---|---|
| BKM0138 | EIQLLES | ITTTGYH | WVRQAP | YIYSSGS | RFTISRDT | GDWHYF | WGQGTM |
| | GGGLVQP | WN (SEQ | GKGLEW | TSYNPSL | SKNTFYLQ | DY | VTVSS |
| | GGSLRLS | ID NO: | VG | KS (SEQ | MNSLRAED | (SEQ | (SEQ ID |
| | CAVSGFS | 308) | (SEQ | ID NO: | TAVYYCAR | ID NO: | NO: 24) |
| | (SEQ ID | | ID NO: | 6001) | (SEQ ID | 504) | |
| | NO: 301) | | 8667) | | NO: 8668) | | |
| BKM0139 | EIQLLES | ITTTGYH | WVRQAP | YIYSSGS | RFTISRDT | GDWHYF | WGQGTM |
| | GGGLVQP | WN (SEQ | GKGLEW | TSYNPSL | SKNTFYLQ | DY | VTVSS |
| | GGSLRLS | ID NO: | VG | KS (SEQ | MNSLRAED | (SEQ | (SEQ ID |
| | CAVSGFS | 308) | (SEQ | ID NO: | TAVYYCAR | ID NO: | NO: 304) |
| | (SEQ ID | | ID NO: | 6001) | (SEQ ID | 504) | |
| | NO: 301) | | 34) | | NO: 36) | | |
| BKM0140 | EIQLLES | ITTTGYH | WVRQAP | YIYSSGS | RFTISRDT | GDWHYF | WGQGTM |
| | GGGLVQP | WN (SEQ | GKGLEW | TSYNPSL | SKNTFYLQ | DY | VTVSS |
| | GGSLRLS | ID NO: | VG | KS (SEQ | MNSLRAED | (SEQ | (SEQ ID |
| | CAVSGFS | 308) | (SEQ | ID NO: | TAVYYCAR | ID NO: | NO: 304) |
| | (SEQ ID | | ID NO: | 6001) | (SEQ ID | 504) | |
| | NO: 301) | | 8676) | | NO: 8677) | | |
| BKM0141 | EIQLLES | ITTTGYH | WVRQAP | YIYSSGS | RFTISRDT | GDWHYF | WGQGTM |
| | GGGLVQP | WN (SEQ | GKGLEW | TSYNPSL | SKNTFYLQ | DY | VTVSS |
| | GGSLRLS | ID NO: | VG | KS (SEQ | MNSLRAED | (SEQ | (SEQ ID |
| | CAVSGFS | 308) | (SEQ | ID NO: | TAVYYCAR | ID NO: | NO: 304) |
| | (SEQ ID | | ID NO: | 6001) | (SEQ ID | 504) | |
| | NO: 301) | | 310) | | NO: 316) | | |
| BKM0142 | EIQLLES | ITTTGYH | WVRQAP | YIYSSGS | RFTISRDT | GDWHYF | WGQGTM |
| | GGGLVQP | WN (SEQ | GKGLEW | TSYAPSL | SKNTFYL | DY | VTVSS |
| | GGSLRLS | ID NO: | VG | KS (SEQ | QMNSLRA | (SEQ | (SEQ ID |
| | CAVSGFS | 308) | (SEQ | ID NO: | EDTAVYY | ID NO: | NO: 304) |
| | (SEQ ID | | ID NO: | 313) | CAR | 504) | |
| | NO: 301) | | 7398) | | (SEQ ID | | |
| | | | | | NO: 316) | | |
| BKM0143 | EIQLLES | ITTTGYH | WVRQAP | YIYSSGS | RFTISRDT | GDWHYF | WGQGTM |
| | GGGLVQP | WN (SEQ | GKGLEW | TSYAPSL | SKNTFYLQ | DY | VTVSS |
| | GGSLRLS | ID NO: | VG | KS (SEQ | MNSLRAED | (SEQ | (SEQ ID |
| | CAVSGFS | 308) | (SEQ | ID NO: | TAVYYCAR | ID NO: | NO: 304) |
| | (SEQ ID | | ID NO: | 313) | (SEQ ID | 504) | |
| | NO: 301) | | 310) | | NO: 8702) | | |
| BKM0144 | EIQLLES | ITTTGYH | WVRQAP | YIYSSGS | RFTISRDT | GDWHYF | WGQGTM |
| | GGGLVQP | WN (SEQ | GKGLEW | TSYAPSL | SKNTFYLQ | DY | VTVSS |
| | GGSLRLS | ID NO: | VG | KS (SEQ | MNSLRAED | (SEQ | (SEQ ID |
| | CAVSGFS | 308) | (SEQ | ID NO: | TAVYYCAR | ID NO: | NO: 304) |
| | (SEQ ID | | ID NO: | 313) | (SEQ ID | 504) | |
| | NO: 301) | | 310) | | NO: 316) | | |
| BKM0145 | EIQLLES | ITTTGYH | WVRQAP | YIYSSGS | RFTISRDT | GDWHYF | WGQGTM |
| | GGGLVQP | WN (SEQ | GKGLEW | TSYAPSL | SKNTFYLQ | DY | VTVSS |
| | GGSLRLS | ID NO: | VG | KS (SEQ | MNSLRAED | (SEQ | (SEQ ID |
| | CAVSGFS | 308) | (SEQ | ID NO: | TAVYYCAR | ID NO: | NO: 304) |
| | (SEQ ID | | ID NO: | 313) | (SEQ ID | 504) | |
| | NO: 301) | | 310) | | NO: 316) | | |

TABLE 23B
Exemplary heavy chain CDRs and FWRs of NKp30-targeting
antigen binding domains of Table 23A (According to the
ABM numbering scheme)

| Ab ID | VHFWR1 | VHCDR1 | VHFWR2 | VHCDR2 | VHFWR3 | VHCDR3 | VHFWR4 |
|---|---|---|---|---|---|---|---|
| BKM0 | EIQLLESG | GFSITTTG | WVRQAPG | YIYSSGS | RFTISRDT | GDWHY | WGQGTM |
| 138 | GGLVQPGG | YHWN | KGLEWVG | TSYNPSL | SKNTFYLQ | FDY | VTVSS |
| | SLRLSCAV | (SEQ ID | (SEQ ID | KS | MNSLRAED | (SEQ ID | (SEQ ID |
| | S (SEQ | NO: 8628) | NO: 310) | (SEQ ID | TAVYYCAR | NO: 504) | NO: 304) |
| | ID NO: | | | NO: | (SEQ ID | | |
| | 8627) | | | 6001) | NO: 316) | | |
| BKM0 | EIQLLESG | GFSITTTG | WVRQAPG | YIYSSGS | RFTISRDT | GDWHY | WGQGTM |
| 139 | GGLVQPGG | YHWN | KGLEWVG | TSYNPSL | SKNTFYLQ | FDY | VTVSS |
| | SLRLSCAV | (SEQ ID | (SEQ ID | KS | MNSLRAED | (SEQ ID | (SEQ ID |
| | S (SEQ | NO: 8630) | NO: 310) | (SEQ ID | TAVYYCAR | NO: 504) | NO: 304) |
| | ID NO: | | | NO: | (SEQ ID | | |
| | 8629) | | | 6001) | NO: 316) | | |
| BKM0 | EIQLLESG | GFSITTTG | WVRQAPG | YIYSSGS | RFTISRDT | GDWHY | WGQGTM |
| 140 | GGLVQPGG | YHWN | KGLEWVG | TSYNPSL | SKNTFYLQ | FDY | VTVSS |
| | SLRLSCAV | (SEQ ID | (SEQ ID | KS | MNSLRAED | (SEQ ID | (SEQ ID |
| | S (SEQ | NO: 8632) | NO: 310) | (SEQ ID | TAVYYCAR | NO: 504) | NO: 304) |
| | ID NO: | | | NO: | (SEQ ID | | |
| | 8631) | | | 6001) | NO: 316) | | |
| BKM0 | EIQLLESG | GFSITTTG | WVRQAPG | YIYSSGS | RFTISRDT | GDWHY | WGQGTM |
| 141 | GGLVQPGG | YHWN | KGLEWVG | TSYNPSL | SKNTFYLQ | FDY | VTVSS |
| | SLRLSCAV | (SEQ ID | (SEQ ID | KS | MNSLRAED | (SEQ ID | (SEQ ID |
| | S (SEQ | NO: 8634) | NO: 310) | (SEQ ID | TAVYYCAR | NO: 504) | NO: 304) |
| | ID NO: | | | NO: | (SEQ ID | | |
| | 8633) | | | 6001) | NO: 316) | | |
| BKM0 | EIQLLESG | GFSITTTG | WVRQAPG | YIYSSGS | RFTISRDT | GDWHY | WGQGTM |
| 142 | GGLVQPGG | YHWN | KGLEWVG | TSYAPSL | SKNTFYLQ | FDY | VTVSS |
| | SLRLSCAV | (SEQ ID | (SEQ ID | KS | MNSLRAED | (SEQ ID | (SEQ ID |
| | S (SEQ | NO: 8636) | NO: 310) | (SEQ ID | TAVYYCAR | NO: 504) | NO: 304) |
| | ID NO: | | | NO: 313) | (SEQ ID | | |
| | 8635) | | | | NO: 316) | | |
| BKM0 | EIQLLESG | GFSITTTG | WVRQAPG | YIYSSGS | RFTISRDT | GDWHY | WGQGTM |
| 143 | GGLVQPGG | YHWN | KGLEWVG | TSYAPSL | SKNTFYLQ | FDY | VTVSS |
| | SLRLSCAV | (SEQ ID | (SEQ ID | KS | MNSLRAED | (SEQ ID | (SEQ ID |
| | S (SEQ | NO: 8638) | NO: 310) | SEQ ID | TAVYYCAR | NO: 504) | NO: 304) |
| | ID NO: | | | NO: 313) | (SEQ ID | | |
| | 8637) | | | | NO: 316) | | |
| BKM0 | EIQLLESG | GFSITTTG | WVRQAPG | YIYSSGS | RFTISRDT | GDWHY | WGQGTM |
| 144 | GGLVQPGG | YHWN | KGLEWVG | TSYAPSL | SKNTFYLQ | FDY | VTVSS |
| | SLRLSCAV | (SEQ ID | (SEQ ID | KS | MNSLRAED | (SEQ ID | (SEQ ID |
| | S (SEQ | NO: 8640) | NO: 310) | (SEQ ID | TAVYYCAR | NO: 504) | NO: 304) |
| | ID NO: | | | NO: 313) | (SEQ ID | | |
| | 8639) | | | | NO: 316) | | |
| BKM0 | EIQLLESG | GFSITTTG | WVRQAPG | YIYSSGS | RFTISRDT | GDWHY | WGQGTM |
| 145 | GGLVQPGG | YHWN | KGLEWVG | TSYAPSL | SKNTFYLQ | FDY | VTVSS |
| | SLRLSCAV | (SEQ ID | SEQ ID | KS | MNSLRAED | (SEQ ID | (SEQ ID |
| | S (SEQ | NO: 8642) | NO: 310) | (SEQ ID | TAVYYCAR | NO: 504) | NO: 304) |
| | ID NO: | | | NO: 313) | (SEQ ID | | |
| | 8641) | | | | NO: 316) | | |

TABLE 24
Exemplary light chain CDRs and FWRs of NKp30-targeting
antigen binding domains

| Ab ID | VLFWR1 | VLCDR1 | VLFWR2 | VLCDR2 | VLFWR3 | VLCDR3 | VLFWR4 |
|---|---|---|---|---|---|---|---|
| BKM0138 | DSVTTQS | SGEKLSD | WYQQRP | ENDRRPS | GVPDRFS | QFWDST | FGGGTK |
| | PLSLPVT | KYVH | GQSPRM | (SEQ ID | GSNSGND | ASAV | VEIK |
| | LGQPASI | (SEQ ID | LIY | NO: 28) | ATLKISR | (SEQ ID | (SEQ |
| | SC (SEQ | NO: 26) | (SEQ | | VEAEDVG | NO: 30) | ID NO: |
| | ID NO: | | ID NO: | | VYFC | | 8685) |
| | 7340) | | 8670) | | (SEQ ID | | |
| | | | | | NO: | | |
| | | | | | 8672) | | |
| BKM0139 | DSVTTQS | SGEKLSD | WYQQRP | ENDRRPS | GVPDRFS | QFWAST | FGGGTK |
| | PLSLPVT | KYVH | GQSPRM | (SEQ ID | GSNSGND | NSAV | VEIK |
| | LGQPASI | (SEQ ID | LIY | NO: 28) | ATLKISR | (SEQ ID | (SEQ |
| | SC (SEQ | NO: 26) | (SEQ | | VEAEDVG | NO: | ID NO: |
| | ID NO: | | ID NO: | | VYFC | 7395) | 8685) |
| | 7340) | | 300) | | (SEQ ID | | |
| | | | | | NO: | | |
| | | | | | 8694) | | |
| BKM0140 | SSETTQP | SGEKLSD | WYQQKP | ENDRRPS | GIPERFS | QFWAST | FGGGTQ |
| | PSVSVSP | KYVH | GQSPVM | (SEQ ID | GSNSGNT | NSAV | LTVL |
| | GQTASIT | (SEQ ID | VIY | NO: 28) | ATLTISG | (SEQ ID | (SEQ |
| | C (SEQ | NO: 26) | (SEQ | | TQAMDEA | NO: | ID NO: |
| | ID NO: | | ID NO: | | DYFC | 8678) | 6080) |
| | 7335) | | 6106) | | (SEQ ID | | |
| | | | | | NO: | | |
| | | | | | 8692) | | |
| BKM0141 | SSETTQP | SGEKLSD | WYQQKP | ENDRRPS | GIPERFS | QFWDST | FGGGTQ |
| | PSVSVSP | KYVH | GQSPVM | (SEQ ID | GSNSGNT | ASAV | LTVL |
| | GQTASIT | (SEQ ID | VIY | NO: 28) | ATLTISG | (SEQ ID | (SEQ |
| | C (SEQ | NO: 26) | (SEQ | | TQAMDEA | NO: 30) | ID NO: |
| | ID NO: | | ID NO: | | DYFC | | 6080) |
| | 7335) | | 6106) | | (SEQ ID | | |
| | | | | | NO: | | |
| | | | | | 8692) | | |
| BKM0142 | DSVTTQS | SGEKLSD | WYQQRP | ENDRRPS | GVPDRFS | QFWDST | FGGGTK |
| | PLSLPVT | KYVH | GQSPRM | (SEQ ID | GSNSGND | NSAV | VEIK |
| | LGQPASI | (SEQ ID | LIY | NO: 28) | ATLKISR | (SEQ ID | (SEQ |
| | SC (SEQ | NO: 26) | (SEQ | | VEAEDVG | NO: 86) | ID NO: |
| | ID NO: | | ID NO: | | VYFC | | 8685) |
| | 7340) | | 300) | | (SEQ ID | | |
| | | | | | NO: | | |
| | | | | | 305) | | |
| BKM0143 | SSETTQP | SGEKLSD | WYQQKP | ENDRRPS | GIPERFS | QFWDST | FGGGTQ |
| | PSVSVSP | KYVH | GQSPVM | (SEQ ID | GSNSGNT | NSAV | LTVL |
| | GQTASIT | (SEQ ID | VIY | NO: 28) | ATLTISG | (SEQ ID | (SEQ |
| | C (SEQ | NO: 26) | (SEQ | | TQAMDEA | NO: 86) | ID NO: |
| | ID NO: | | ID NO: | | DYFC | | 6080) |
| | 7335) | | 6106) | | (SEQ ID | | |
| | | | | | NO: | | |
| | | | | | 8692) | | |
| BKM0144 | DSVTTQS | SGEKLSD | WYQQRP | ENDRRPS | GVPDRFS | QFWAST | FGGGTK |
| | PLSLPVT | KYVH | GQSPRM | (SEQ ID | GSNSGND | ASAV | VEIK |
| | LGQPASI | (SEQ ID | LIY | NO: 28) | ATLKISR | (SEQ ID | (SEQ |
| | SC (SEQ | NO: 26) | (SEQ | | VEAEDVG | NO: | ID NO: |
| | ID NO: | | ID NO: | | VYFC | 330) | 8685) |
| | 7340) | | 300) | | (SEQ ID | | |
| | | | | | NO: | | |
| | | | | | 305) | | |
| BKM0145 | SSETTQP | SGEKLSD | WYQQKP | ENDRRPS | GIPERFS | QFWAST | FGGGTQ |
| | PSVSVSP | KYVH | GQSPVM | (SEQ ID | GSNSGNT | ASAV | LTVL |
| | GQTASIT | (SEQ ID | VIY | NO: 28) | ATLTISG | (SEQ ID | (SEQ |
| | C (SEQ | NO: 26) | (SEQ | | TQAMDEA | NO: | ID NO: |
| | ID NO: | | ID NO: | | DYFC | 330) | 6080) |
| | 7335) | | 6106) | | (SEQ ID | | |
| | | | | | NO: | | |
| | | | | | 8692) | | |

TABLE 25
ExemplaryVariable regions of NKp30-targeting antigen binding domains

| SEQ ID | | | |
|---|---|---|---|
| NO | Ab ID | Description | Sequence |
| SEQ ID | 9G1-HC | 9G1 heavy chain | QIQLQESGPGLVKPSQSLSLTCSVTGFSINTGGYHWN |
| NO: 6121 | | variable region | WIRQFPGKKLEWMGYIYSSGSTSYNPSLKSRISITRD |
| | | | TSKNQFFLQLNSVTTEDTATYYCARGNWHYFDFWGQG |
| | | | TMVTVSS |
| SEQ ID | 15H6-HC | 15H6 heavy chain | QIQLQESGPGLVKPSQSLSLTCSVTGFSINTGGYHWN |
| NO: 6122 | | variable region | WIRQFPGKKLEWMGYIYSSGTTRYNPSLKSRISITRD |
| | | | TSKNQFFLQLNSVTPEDTATYYCTRGNWHYFDYWGQG |
| | | | TLVAVSS |
| SEQ ID | 9G1-HC_1 | 9G1 heavy chain | QIQLQESGPGLVKPSETLSLTCTVSGFSINTGGYHWN |
| NO: 6123 | | variable region | WIRQPAGKGLEWIGYIYSSGSTSYNPSLKSRVTMSRD |
| | | humanized Variant 1 | TSKNQFSLKLSSVTAADTAVYYCARGNWHYFDFWGQG |
| | | | TMVTVSS |
| SEQ ID | 9G1-HC_2 | 9G1 heavy chain | QIQLQESGPGLVKPSQTLSLTCTVSGFSINTGGYHWN |
| NO: 6124 | | variable region | WIRQHPGKGLEWIGYIYSSGSTSYNPSLKSLVTISRD |
| | | humanized Variant 2 | TSKNQFSLKLSSVTAADTAVYYCARGNWHYFDFWGQG |
| | | | TMVTVSS |
| SEQ ID | 9G1-HC_3 | 9G1 heavy chain | EIQLLESGGGLVQPGGSLRLSCAVSGFSINTGGYHWN |
| NO: 6125 | | variable region | WVRQAPGKGLEWVGYIYSSGSTSYNPSLKSRFTISRD |
| | | humanized Variant 3 | TSKNTFYLQMNSLRAEDTAVYYCARGNWHYFDFWGQG |
| | | | TMVTVSS |
| SEQ ID | 9G1-HC_4 | 9G1 heavy chain | QIQLVQSGAEVKKPGSSVKVSCKVSGFSINTGGYHWN |
| NO: 6126 | | variable region | WVRQAPGQGLEWMGYIYSSGSTSYNPSLKSRVTITRD |
| | | humanized Variant 4 | TSTNTFYMELSSLRSEDTAVYYCARGNWHYFDFWGQG |
| | | | TMVTVSS |
| SEQ ID | 9G1-HC_5 | 9G1 heavy chain | EIQLVESGGGLVQPGGSLRLSCAVSGFSINTGGYHWN |
| NO: 6127 | | variable region | WVRQAPGKGLEWVGYIYSSGSTSYNPSLKSRFTISRD |
| | | humanized Variant 5 | TAKNSFYLQMNSLRAEDTAVYYCARGNWHYFDFWGQG |
| | | | TMVTVSS |
| SEQ ID | 9G1-HC_6 | 9G1 heavy chain | QIQLVQSGAEVKKPGASVKVSCKVSGFSINTGGYHWN |
| NO: 6128 | | variable region | WVRQAPGQGLEWMGYIYSSGSTSYNPSLKSRVTMTRD |
| | | humanized Variant 6 | TSTNTFYMELSSLRSEDTAVYYCARGNWHYFDFWGQG |
| | | | TMVTVSS |
| SEQ ID | 15H6-HC_1 | 15H6 heavy chain | QIQLQESGPGLVKPSQTLSLTCTVSGFSINTGGYHWN |
| NO: 6129 | | variable region | WIRQHPGKGLEWIGYIYSSGTTRYNPSLKSLVTISRD |
| | | humanized Variant 1 | TSKNQFSLKLSSVTAADTAVYYCARGNWHYFDYWGQG |
| | | | TLVTVSS |
| SEQ ID | 15H6-HC_2 | 15H6 heavy chain | QIQLQESGPGLVKPSETLSLTCTVSGFSINTGGYHWN |
| NO: 6130 | | variable region | WIRQPAGKGLEWIGYIYSSGTTRYNPSLKSRVTMSRD |
| | | humanized Variant 2 | TSKNQFSLKLSSVTAADTAVYYCARGNWHYFDYWGQG |
| | | | TLVTVSS |
| SEQ ID | 15H6-HC_3 | 15H6 heavy chain | EIQLLESGGGLVQPGGSLRLSCAVSGFSINTGGYHWN |
| NO: 6131 | | variable region | WVRQAPGKGLEWVGYIYSSGTTRYNPSLKSRFTISRD |
| | | humanized Variant 3 | TSKNTFYLQMNSLRAEDTAVYYCARGNWHYFDYWGQG |
| | | | TLVTVSS |
| SEQ ID | 15H6-HC 4 | 15H6 heavy chain | QIQLVESGGGLVKPGGSLRLSCAVSGFSINTGGYHWN |
| NO: 6132 | | variable region | WIRQAPGKGLEWVGYIYSSGTTRYNPSLKSRFTISRD |
| | | humanized Variant 4 | TAKNSFYLQMNSLRAEDTAVYYCARGNWHYFDYWGQG |
| | | | TLVTVSS |
| SEQ ID | 15H6-HC_5 | 15H6 heavy chain | QIQLVQSGAEVKKPGASVKVSCKVSGFSINTGGYHWN |
| NO: 6133 | | variable region | WVRQAPGQGLEWMGYIYSSGTTRYNPSLKSRVTMTRD |
| | | humanized Variant 5 | TSTNTFYMELSSLRSEDTAVYYCARGNWHYFDYWGQG |
| | | | TLVTVSS |
| SEQ ID | 15H6-HC_6 | 15H6 heavy chain | EIQLVQSGAEVKKPGATVKISCKVSGFSINTGGYHWN |
| NO: 6134 | | variable region | WVQQAPGKGLEWMGYIYSSGTTRYNPSLKSRVTITRD |
| | | humanized Variant 6 | TSTNTFYMELSSLRSEDTAVYYCARGNWHYFDYWGQG |
| | | | TLVTVSS |
| SEQ ID | 9G1-LC | 9G1 light chain | SYTLTQPPLLSVALGHKATITCSGERLSDKYVHWYQQ |
| NO: 7294 | | variable region | KPGRAPVMVIYENDKRPSGIPDQFSGSNSGNIATLTI |
| | | | SKAQAGYEADYYCQSWDSTNSAVFGSGTQLTVL |
| SEQ ID | 15H6-LC | 15H6 light chain | SYTLTQPPSLSVAPGQKATIICSGENLSDKYVHWYQQ |
| NO: 6136 | | variable region | KPGRAPVMVIYENEKRPSGIPDQFSGSNSGNIATLTI |
| | | | SKAQPGSEADYYCHYWESINSVVFGSGTHLTVL |
| SEQ ID | 9G1-LC_1 | 9G1 light chain | QSVTTQPPSVSGAPGQRVTISCSGERLSDKYVHWYQQ |
| NO: 6137 | | variable region | LPGTAPKMLIYENDKRPSGVPDRFSGSNSGNSASLAI |
| | | humanized Variant 1 | TGLQAEDEADYYCQSWDSTNSAVFGGGTQLTVL |
| SEQ ID | 9G1-LC_2 | 9G1 light chain | QSVTTQPPSASGTPGQRVTISCSGERLSDKYVHWYQQ |
| NO: 6138 | | variable region | LPGTAPKMLIYENDKRPSGVPDRFSGSNSGNSASLAI |
| | | humanized Variant 2 | SGLQSEDEADYYCQSWDSTNSAVFGGGTQLTVL |
| SEQ ID | 9G1-LC_3 | 9G1 light chain | QSVTTQPPSASGTPGQRVTISCSGERLSDKYVHWYQQ |
| NO: 6139 | | variable region | LPGTAPKMLIYENDKRPSGVPDRFSGSNSGNSASLAI |
| | | humanized Variant 3 | SGLRSEDEADYYCQSWDSTNSAVFGGGTQLTVL |
| SEQ ID | 9G1-LC_4 | 9G1 light chain | SSETTQPHSVSVATAQMARITCSGERLSDKYVHWYQQ |
| NO: 6140 | | variable region | KPGQDPVMVIYENDKRPSGIPERFSGSNPGNTATLTI |
| | | humanized Variant 4 | SRIEAGDEADYYCQSWDSTNSAVFGGGTQLTVL |
| SEQ ID | 9G1-LC_5 | 9G1 light chain | DIQMTQSPSTLSASVGDRVTITCSGERLSDKYVHWYQ |
| NO: 6141 | | variable region | QKPGKAPKMLIYENDKRPSGVPSRFSGSNSGNEATLT |
| | | humanized Variant 5 | ISSLQPDDFATYYCQSWDSTNSAVFGQGTKVEIK |
| SEQ ID | 15H6-LC_1 | 15H6 light chain | QYVLTQPPSASGTPGQRVTISCSGENLSDKYVHWYQQ |
| NO: 6142 | | variable region | LPGTAPKMLIYENEKRPSGVPDRFSGSNSGNSASLAI |
| | | humanized Variant 1 | SGLQSEDEADYYCHYWESINSVVFGEGTELTVL |
| SEQ ID | 15H6-LC_2 | 15H6 light chain | QYVLTQPPSASGTPGQRVTISCSGENLSDKYVHWYQQ |
| NO: 6143 | | variable region | LPGTAPKMLIYENEKRPSGVPDRFSGSNSGNSASLAI |
| | | humanized Variant 2 | SGLRSEDEADYYCHYWESINSVVFGEGTELTVL |
| SEQ ID | 15H6-LC_3 | 15H6 light chain | SYELTQPPSVSVSPGQTASITCSGENLSDKYVHWYQQ |
| NO: 6144 | | variable region | KPGQSPVMVIYENEKRPSGIPERFSGSNSGNTATLTI |
| | | humanized Variant 3 | SGTQAMDEADYYCHYWESINSVVFGEGTELTVL |
| SEQ ID | 15H6-LC_4 | 15H6 light chain | DYVLTQSPLSLPVTPGEPASISCSGENLSDKYVHWYL |
| NO: 6145 | | variable region | QKPGQSPQMLIYENEKRPSGVPDRFSGSNSGNDATLK |
| | | humanized Variant 4 | ISRVEAEDVGVYYCHYWESINSVVFGQGTKVEIK |
| SEQ ID | 15H6-LC_5 | 15H6 light chain | AYQLTQSPSSLSASVGDRVTITCSGENLSDKYVHWYQ |
| NO: 6146 | | variable region | QKPGKAPKMLIYENEKRPSGVPSRFSGSNSGNDATLT |
| | | humanized Variant 5 | ISSLQPEDFATYYCHYWESINSVVFGQGTKVEIK |
| SEQ ID | 15H6-LC_6 | 15H6 light chain | EYVLTQSPATLSVSPGERATLSCSGENLSDKYVHWYQ |
| NO: 6147 | | variable region | QKPGQAPRMLIYENEKRPSGIPARFSGSNSGNEATLT |
| | | humanized Variant 6 | ISSLQSEDFAVYYCHYWESINSVVFGQGTKVEIK |
| SEQ ID | 9D9-HC | 9D9 heavy chain | QIQLQESGPGLVKPSQSLSLSCSVTGFSINTGGYHWN |
| NO: 7295 | | variable region | WIRQFPGKKVEWMGYIYSSGTTKYNPSLKSRISITRD |
| | | | TSKNQFFLQLNSVTTEDTATYYCARGDWHYFDYWGQG |
| | | | TMVAVSS |
| SEQ ID | 9D9-LC | 9D9 light chain | SYTLTQPPLVSVALGQKATIICSGENLSDKYVHWYQQ |
| NO: 7296 | | variable region | KPGRAPVMVIYENDKRPSGIPDQFSGSNSGNIATLTI |
| | | | SKAQAGYEADYYCHCWDSTNSAVFGSGTHLTVL |
| SEQ ID | 3A12-HC | 3A12 heavy chain | QIQLQESGPGLVKPSQSLSLTCSVTGFSINTGGYHWN |
| NO: 7297 | | variable region | WIRQFPGKKLEWMGYIYSSGSTRYNPSLKSRFSITRD |
| | | | TSKNQFFLQLNSVTTEDTATYYCTRGNWHYFDYWGQG |
| | | | TLVAVSS |
| SEQ ID | 3A12-LC | 3A12 light chain | SYTLTQPPLVSVALGQKATIICSGENLSDKYVHWYQQ |
| NO: 7296 | | variable region | KPGRAPVMVIYENDKRPSGIPDQFSGSNSGNIATLTI |
| | | | SKAQAGYEADYYCHCWDSTNSAVFGSGTHLTVL |
| SEQ ID | 12D10-HC | 12D10 heavy chain | QIQLQESGPGLVKPSQSLSLTCSVTGFSINTGGYHWN |
| NO: 6122 | | variable region | WIRQFPGKKLEWMGYIYSSGTTRYNPSLKSRISITRD |
| | | | TSKNQFFLQLNSVTPEDTATYYCTRGNWHYFDYWGQG |
| | | | TLVAVSS |
| SEQ ID | 12D10-LC | 12D10 light chain | SYTLTQPPSLSVAPGQKATIICSGENLSDKYVHWYQQ |
| NO: 6136 | | variable region | KPGRAPVMVIYENEKRPSGIPDQFSGSNSGNIATLTI |
| | | | SKAQPGSEADYYCHYWESINSVVFGSGTHLTVL |
| SEQ ID | 15E1-HC | 15E1 heavy chain | QIQLQESGPGLVKPSQSLSLSCSVTGFSITTTGYHWN |
| NO: 7298 | | variable region | WIRQFPGKKLEWMGYIYSSGSTSYNPSLKSRFSITRD |
| | | | TSKNQFFLQLNSVTTEDTATYYCARGDWHYFDYWGPG |
| | | | TMVTVSS |
| SEQ ID | 15E1-LC | 15E1 light chain | SFTLTQPPLVSVAVGQVATITCSGEKLSDKYVHWYQQ |
| NO: 7299 | | variable region | KPGRAPVMVIYENDRRPSGIPDQFSGSNSGNIASLTI |
| | | | SKAQAGDEADYFCQFWDSTNSAVFGGGTQLTVL |
| SEQ ID | 15E1 Human- | 15E1 heavy chain | QIQLQESGPGLVKPSQTLSLTCTVSGFSITTTGYHWN |
| NO: 7300 | ized | variable region | WIRQHPGKGLEWIGYIYSSGSTSYNPSLKSLVTISRD |
| | variant_VH1 | humanized Variant 1 | TSKNQFSLKLSSVTAADTAVYYCARGDWHYFDYWGQG |
| | | | TMVTVSS |
| SEQ ID | 15E1 Human- | 15E1 heavy chain | QIQLVESGGGLVKPGGSLRLSCAVSGFSITTTGYHWN |
| NO: 7301 | ized | variable region | WIRQAPGKGLEWVGYIYSSGSTSYNPSLKSRFTISRD |
| | variantVH2 | humanized Variant 2 | TAKNSFYLQMNSLRAEDTAVYYCARGDWHYFDYWGQG |
| | | | TMVTVSS |
| SEQ ID | 15E1 Human- | 15E1 heavy chain | EIQLLESGGGLVQPGGSLRLSCAVSGFSITTTGYHWN |
| NO: 8030 | ized | variable region | WVRQAPGKGLEWVGYIYSSGSTSYNPSLKSRFTISRD |
| | variant_VH3 | humanized Variant 3 | TSKNTFYLQMNSLRAEDTAVYYCARGDWHYFDYWGQG |
| | (BJM0407 | | TMVTVSS |
| | VH and | | |
| | BJM0411 | | |
| | VH) | | |
| SEQ ID | 15E1 Human- | 15E1 heavy chain | EIQLVESGGGLVQPGGSLRLSCAVSGFSITTTGYHWN |
| NO: 7303 | ized | variable region | WVRQAPGKGLEWVGYIYSSGSTSYNPSLKSRFTISRD |
| | variantVH4 | humanized Variant 4 | TAKNSFYLQMNSLRAEDTAVYYCARGDWHYFDYWGQG |
| | | | TMVTVSS |
| SEQ ID | 15E1 Human- | 15E1 heavy chain | QIQLVQSGAEVKKPGASVKVSCKVSGFSITTTGYHWN |
| NO: 7304 | ized | variable region | WVRQAPGQGLEWMGYIYSSGSTSYNPSLKSRVTMTRD |
| | variant_VH5 | humanized Variant 5 | TSTNTFYMELSSLRSEDTAVYYCARGDWHYFDYWGQG |
| | | | TMVTVSS |
| SEQ ID | 15E1 Human- | 15E1 light chain | SSETTQPPSVSVSPGQTASITCSGEKLSDKYVHWYQQ |
| NO: 7305 | ized | variable region | KPGQSPVMVIYENDRRPSGIPERFSGSNSGNTATLTI |
| | variantVL1 | humanized Variant 1 | SGTQAMDEADYFCQFWDSTNSAVFGGGTQLTVL |
| | (BJM0407 | | |
| | VL) | | |
| SEQ ID | 15E1 Human- | 15E1 light chain | SSETTQPHSVSVATAQMARITCSGEKLSDKYVHWYQQ |
| NO: 7306 | ized | variable region | KPGQDPVMVIYENDRRPSGIPERFSGSNPGNTATLTI |
| | variant_VL2 | humanized Variant 2 | SRIEAGDEADYFCQFWDSTNSAVFGGGTQLTVL |
| SEQ ID | 15E1 Human- | 15E1 light chain | QSVTTQPPSASGTPGQRVTISCSGEKLSDKYVHWYQQ |
| NO: 7307 | ized | variable region | LPGTAPKMLIYENDRRPSGVPDRFSGSNSGNSASLAI |
| | variantVL3 | humanized Variant 3 | SGLRSEDEADYFCQFWDSTNSAVFGGGTQLTVL |
| SEQ ID | 15E1 Human- | 15E1 light chain | QSVTTQPPSVSGAPGQRVTISCSGEKLSDKYVHWYQQ |
| NO: 7308 | ized | variable region | LPGTAPKMLIYENDRRPSGVPDRFSGSNSGNSASLAI |
| | variantVL4 | humanized Variant 4 | TGLQAEDEADYFCQFWDSTNSAVFGGGTQLTVL |
| SEQ ID | 15E1 Human- | 15E1 light chain | DSVTTQSPLSLPVTLGQPASISCSGEKLSDKYVHWYQ |
| NO: 13 | ized | variable region | QRPGQSPRMLIYENDRRPSGVPDRFSGSNSGNDATLK |
| | variantVL5 | humanized Variant 5 | ISRVEAEDVGVYFCQFWDSTNSAVFGGGTKVEIK |
| | (BJM0411 | | |
| | VL) | | |
| SEQ ID | BKM0138 | BKM0138 heavy | EIQLLESGGGLVQPGGSLRLSCAVSGFSITTTGYHWN |
| NO: 8030 | VH | chain Variable | WVRQAPGKGLEWVGYIYSSGSTSYNPSLKSRFTISRD |
| | | region | TSKNTFYLQMNSLRAEDTAVYYCARGDWHYFDYWGQG |
| | | | TMVTVSS |
| SEQ ID | BKM0139 | BKM0139 heavy | EIQLLESGGGLVQPGGSLRLSCAVSGFSITTTGYHWN |
| NO: 8030 | VH | chain Variable | WVRQAPGKGLEWVGYIYSSGSTSYNPSLKSRFTISRD |
| | | region | TSKNTFYLQMNSLRAEDTAVYYCARGDWHYFDYWGQG |
| | | | TMVTVSS |
| SEQ ID | BKM0140 | BKM0140 heavy | EIQLLESGGGLVQPGGSLRLSCAVSGFSITTTGYHWN |
| NO: 8030 | VH | chain Variable | WVRQAPGKGLEWVGYIYSSGSTSYNPSLKSRFTISRD |
| | | region | TSKNTFYLQMNSLRAEDTAVYYCARGDWHYFDYWGQG |
| | | | TMVTVSS |
| SEQ ID | BKM0141 | BKM0141 heavy | EIQLLESGGGLVQPGGSLRLSCAVSGFSITTTGYHWN |
| NO: 8030 | VH | chain Variable | WVRQAPGKGLEWVGYIYSSGSTSYNPSLKSRFTISRD |
| | | region | TSKNTFYLQMNSLRAEDTAVYYCARGDWHYFDYWGQG |
| | | | TMVTVSS |
| SEQ ID | BKM0142 | BKM0142 heavy | EIQLLESGGGLVQPGGSLRLSCAVSGFSITTTGYHWN |
| NO: 7302 | VH | chain Variable | WVRQAPGKGLEWVGYIYSSGSTSYAPSLKSRFTISRD |
| | | region | TSKNTFYLQMNSLRAEDTAVYYCARGDWHYFDYWGQG |
| | | | TMVTVSS |
| SEQ ID | BKM0143 | BKM0143 heavy | EIQLLESGGGLVQPGGSLRLSCAVSGFSITTTGYHWN |
| NO: 7302 | VH | chain Variable | WVRQAPGKGLEWVGYIYSSGSTSYAPSLKSRFTISRD |
| | | region | TSKNTFYLQMNSLRAEDTAVYYCARGDWHYFDYWGQG |
| | | | TMVTVSS |
| SEQ ID | BKM0144 | BKM0144 heavy | EIQLLESGGGLVQPGGSLRLSCAVSGFSITTTGYHWN |
| NO: 7302 | VH | chain Variable | WVRQAPGKGLEWVGYIYSSGSTSYAPSLKSRFTISRD |
| | | region | TSKNTFYLQMNSLRAEDTAVYYCARGDWHYFDYWGQG |
| | | | TMVTVSS |
| SEQ ID | BKM0145 | BKM0145 heavy | EIQLLESGGGLVQPGGSLRLSCAVSGFSITTTGYHWN |
| NO: 7302 | VH | chain Variable | WVRQAPGKGLEWVGYIYSSGSTSYAPSLKSRFTISRD |
| | | region | TSKNTFYLQMNSLRAEDTAVYYCARGDWHYFDYWGQG |
| | | | TMVTVSS |
| SEQ ID | BKM0138 | BKM0138 light | DSVTTQSPLSLPVTLGQPASISCSGEKLSDKYVHWYQ |
| NO: 8691 | VL | chain Variable | QRPGQSPRMLIYENDRRPSGVPDRFSGSNSGNDATLK |
| | | region | VEAEDVGVYFCQFWDSTASAVFGGGTKVEIKISR |
| SEQ ID | BKM0139 | BKM0139 light | DSVTTQSPLSLPVTLGQPASISCSGEKLSDKYVHWYQ |
| NO: 380 | VL | chain Variable | QRPGQSPRMLIYENDRRPSGVPDRFSGSNSGNDATLK |
| | | region | ISRVEAEDVGVYFCQFWASTNSAVFGGGTKVEIK |
| SEQ ID | BKM0140 | BKM0140 light | SSETTQPPSVSVSPGQTASITCSGEKLSDKYVHWYQQ |
| NO: 381 | VL | chain Variable | KPGQSPVMVIYENDRRPSGIPERFSGSNSGNTATLTI |
| | | region | SGTQAMDEADYFCQFWASTNSAVFGGGTQLTVL |
| SEQ ID | BKM0141 | BKM0141 light | SSETTQPPSVSVSPGQTASITCSGEKLSDKYVHWYQQ |
| NO: 382 | VL | chain Variable | KPGQSPVMVIYENDRRPSGIPERFSGSNSGNTATLTI |
| | | region | SGTQAMDEADYFCQFWDSTASAVFGGGTQLTVL |
| SEQ ID | BKM0142 | BKM0142 light | DSVTTQSPLSLPVTLGQPASISCSGEKLSDKYVHWYQ |
| NO: 13 | VL | chain Variable | QRPGQSPRMLIYENDRRPSGVPDRFSGSNSGNDATLK |
| | | region | ISRVEAEDVGVYFCQFWDSTNSAVFGGGTKVEIK |
| SEQ ID | BKM0143 | BKM0143 light | SSETTQPPSVSVSPGQTASITCSGEKLSDKYVHWYQQ |
| NO: 7305 | VL | chain Variable | KPGQSPVMVIYENDRRPSGIPERFSGSNSGNTATLTI |
| | | region | SGTQAMDEADYFCQFWDSTNSAVFGGGTQLTVL |
| SEQ ID | BKM0144 | BKM0144 light | DSVTTQSPLSLPVTLGQPASISCSGEKLSDKYVHWYQ |
| NO: 384 | VL | chain Variable | QRPGQSPRMLIYENDRRPSGVPDRFSGSNSGNDATLK |
| | | region | ISRVEAEDVGVYFCQFWASTASAVFGGGTKVEIK |
| SEQ ID | BKM0145 | BKM0145 light | SSETTQPPSVSVSPGQTASITCSGEKLSDKYVHWYQQ |
| NO: 385 | VL | chain Variable | KPGQSPVMVIYENDRRPSGIPERFSGSNSGNTATLTI |
| | | region | SGTQAMDEADYFCQFWASTASAVFGGGTQLTVL |

TABLE 26
Exemplary NKp30-targeting antigen binding domains/antibody molecules

| SEQ ID | | | |
|---|---|---|---|
| NO | Ab ID | Description | Sequence |
| SEQ ID | Ch(anti- | 9G1 heavy | QIQLQESGPGLVKPSQSLSLTCSVTGFSINTGGYHWNWIRQF |
| NO: | NKp30 | chain | PGKKLEWMGYIYSSGSTSYNPSLKSRISITRDTSKNQFFLQL |
| 6148 | 9G1)HC | | NSVTTEDTATYYCARGNWHYFDFWGQGTMVTVSSASTKGPSV |
| | N297A | | FPLAPSSKSTSGGTAALGCLVKDYFPEPVTVSWNSGALTSGV |
| | | | HTFPAVLQSSGLYSLSSVVTVPSSSLGTQTYICNVNHKPSNT |
| | | | KVDKRVEPKSCDKTHTCPPCPAPELLGGPSVFLFPPKPKDTL |
| | | | MISRTPEVTCVVVDVSHEDPEVKFNWYVDGVEVHNAKTKPRE |
| | | | EQYASTYRVVSVLTVLHQDWLNGKEYKCKVSNKALPAPIEKT |
| | | | ISKAKGQPREPQVCTLPPSREEMTKNQVSLSCAVKGFYPSDI |
| | | | AVEWESNGQPENNYKTTPPVLDSDGSFFLVSKLTVDKSRWQQ |
| | | | GNVFSCSVMHEALHNHYTQKSLSLSPGK |
| SEQ ID | Ch(anti- | 9G1 heavy | QIQLQESGPGLVKPSQSLSLTCSVTGFSINTGGYHWNWIRQF |
| NO: | NKp30 | chain | PGKKLEWMGYIYSSGSTSYNPSLKSRISITRDTSKNQFFLQL |
| 4688 | 9G1)HC | | NSVTTEDTATYYCARGNWHYFDFWGQGTMVTVSSASTKGPSV |
| | N297A | | FPLAPSSKSTSGGTAALGCLVKDYFPEPVTVSWNSGALTSGV |
| | | | HTFPAVLQSSGLYSLSSVVTVPSSSLGTQTYICNVNHKPSNT |
| | | | KVDKRVEPKSCDKTHTCPPCPAPELLGGPSVFLFPPKPKDTL |
| | | | MISRTPEVTCVVVDVSHEDPEVKFNWYVDGVEVHNAKTKPRE |
| | | | EQYASTYRVVSVLTVLHQDWLNGKEYKCKVSNKALPAPIEKT |
| | | | ISKAKGQPREPQVCTLPPSREEMTKNQVSLSCAVKGFYPSDI |
| | | | AVEWESNGQPENNYKTTPPVLDSDGSFFLVSKLTVDKSRWQQ |
| | | | GNVFSCSVMHEALHNHYTQKSLSLSP |
| SEQ ID | Ch(anti- | 9G1 heavy | QIQLQESGPGLVKPSQSLSLTCSVTGFSINTGGYHWNWIRQF |
| NO: | NKp30 | chain | PGKKLEWMGYIYSSGSTSYNPSLKSRISITRDTSKNQFFLQL |
| 6149 | 9G1)HC | | NSVTTEDTATYYCARGNWHYFDFWGQGTMVTVSSASTKGPSV |
| | | | FPLAPSSKSTSGGTAALGCLVKDYFPEPVTVSWNSGALTSGV |
| | | | HTFPAVLQSSGLYSLSSVVTVPSSSLGTQTYICNVNHKPSNT |
| | | | KVDKRVEPKSCDKTHTCPPCPAPELLGGPSVFLFPPKPKDTL |
| | | | MISRTPEVTCVVVDVSHEDPEVKFNWYVDGVEVHNAKTKPRE |
| | | | EQYNSTYRVVSVLTVLHQDWLNGKEYKCKVSNKALPAPIEKT |
| | | | ISKAKGQPREPQVCTLPPSREEMTKNQVSLSCAVKGFYPSDI |
| | | | AVEWESNGQPENNYKTTPPVLDSDGSFFLVSKLTVDKSRWQQ |
| | | | GNVFSCSVMHEALHNHYTQKSLSLSPGK |
| SEQ ID | Ch(anti- | 9G1 heavy | QIQLQESGPGLVKPSQSLSLTCSVTGFSINTGGYHWNWIRQF |
| NO: | NKp30 | chain | PGKKLEWMGYIYSSGSTSYNPSLKSRISITRDTSKNQFFLQL |
| 4689 | 9G1)HC | | NSVTTEDTATYYCARGNWHYFDFWGQGTMVTVSSASTKGPSV |
| | | | FPLAPSSKSTSGGTAALGCLVKDYFPEPVTVSWNSGALTSGV |
| | | | HTFPAVLQSSGLYSLSSVVTVPSSSLGTQTYICNVNHKPSNT |
| | | | KVDKRVEPKSCDKTHTCPPCPAPELLGGPSVFLFPPKPKDTL |
| | | | MISRTPEVTCVVVDVSHEDPEVKFNWYVDGVEVHNAKTKPRE |
| | | | EQYNSTYRVVSVLTVLHQDWLNGKEYKCKVSNKALPAPIEKT |
| | | | ISKAKGQPREPQVCTLPPSREEMTKNQVSLSCAVKGFYPSDI |
| | | | AVEWESNGQPENNYKTTPPVLDSDGSFFLVSKLTVDKSRWQQ |
| | | | GNVFSCSVMHEALHNHYTQKSLSLSP |
| SEQ ID | Ch(anti- | 9G1 light | SYTLTQPPLLSVALGHKATITCSGERLSDKYVHWYQQKPGRA |
| NO: | NKp30 | chain | PVMVIYENDKRPSGIPDQFSGSNSGNIATLTISKAQAGYEAD |
| 6150 | 9G1)LC | | YYCQSWDSTNSAVFGSGTQLTVLGQPKANPTVTLFPPSSEEL |
| | | | QANKATLVCLISDFYPGAVTVAWKADGSPVKAGVETTKPSKQ |
| | | | SNNKYAASSYLSLTPEQWKSHRSYSCQVTHEGSTVEKTVAPT |
| | | | ECS |
| SEQ ID | Ch(anti- | 15H6 | QIQLQESGPGLVKPSQSLSLTCSVTGFSINTGGYHWNWIRQF |
| NO: | NKp30 | heavy | PGKKLEWMGYIYSSGTTRYNPSLKSRISITRDTSKNQFFLQL |
| 6151 | 15H6)HC | chain | NSVTPEDTATYYCTRGNWHYFDYWGQGTLVAVSSASTKGPSV |
| | N297A | | FPLAPSSKSTSGGTAALGCLVKDYFPEPVTVSWNSGALTSGV |
| | | | HTFPAVLQSSGLYSLSSVVTVPSSSLGTQTYICNVNHKPSNT |
| | | | KVDKRVEPKSCDKTHTCPPCPAPELLGGPSVFLFPPKPKDTL |
| | | | MISRTPEVTCVVVDVSHEDPEVKFNWYVDGVEVHNAKTKPRE |
| | | | EQYASTYRVVSVLTVLHQDWLNGKEYKCKVSNKALPAPIEKT |
| | | | ISKAKGQPREPQVCTLPPSREEMTKNQVSLSCAVKGFYPSDI |
| | | | AVEWESNGQPENNYKTTPPVLDSDGSFFLVSKLTVDKSRWQQ |
| | | | GNVFSCSVMHEALHNHYTQKSLSLSPGK |
| SEQ ID | Ch(anti- | 15H6 | QIQLQESGPGLVKPSQSLSLTCSVTGFSINTGGYHWNWIRQF |
| NO: | NKp30 | heavy | PGKKLEWMGYIYSSGTTRYNPSLKSRISITRDTSKNQFFLQL |
| 4690 | 15H6)HC | chain | NSVTPEDTATYYCTRGNWHYFDYWGQGTLVAVSSASTKGPSV |
| | N297A | | FPLAPSSKSTSGGTAALGCLVKDYFPEPVTVSWNSGALTSGV |
| | | | HTFPAVLQSSGLYSLSSVVTVPSSSLGTQTYICNVNHKPSNT |
| | | | KVDKRVEPKSCDKTHTCPPCPAPELLGGPSVFLFPPKPKDTL |
| | | | MISRTPEVTCVVVDVSHEDPEVKFNWYVDGVEVHNAKTKPRE |
| | | | EQYASTYRVVSVLTVLHQDWLNGKEYKCKVSNKALPAPIEKT |
| | | | ISKAKGQPREPQVCTLPPSREEMTKNQVSLSCAVKGFYPSDI |
| | | | AVEWESNGQPENNYKTTPPVLDSDGSFFLVSKLTVDKSRWQQ |
| | | | GNVFSCSVMHEALHNHYTQKSLSLSP |
| SEQ ID | Ch(anti- | 15H6 | QIQLQESGPGLVKPSQSLSLTCSVTGFSINTGGYHWNWIRQF |
| NO: | NKp30 | heavy | PGKKLEWMGYIYSSGTTRYNPSLKSRISITRDTSKNQFFLQL |
| 6152 | 15H6)HC | chain | NSVTPEDTATYYCTRGNWHYFDYWGQGTLVAVSSASTKGPSV |
| | (hole) | | FPLAPSSKSTSGGTAALGCLVKDYFPEPVTVSWNSGALTSGV |
| | | | HTFPAVLQSSGLYSLSSVVTVPSSSLGTQTYICNVNHKPSNT |
| | | | KVDKRVEPKSCDKTHTCPPCPAPELLGGPSVFLFPPKPKDTL |
| | | | MISRTPEVTCVVVDVSHEDPEVKFNWYVDGVEVHNAKTKPRE |
| | | | EQYNSTYRVVSVLTVLHQDWLNGKEYKCKVSNKALPAPIEKT |
| | | | ISKAKGQPREPQVCTLPPSREEMTKNQVSLSCAVKGFYPSDI |
| | | | AVEWESNGQPENNYKTTPPVLDSDGSFFLVSKLTVDKSRWQQ |
| | | | GNVFSCSVMHEALHNHYTQKSLSLSPGK |
| SEQ ID | Ch(anti- | 15H6 | QIQLQESGPGLVKPSQSLSLTCSVTGFSINTGGYHWNWIRQF |
| NO: | NKp30 | heavy | PGKKLEWMGYIYSSGTTRYNPSLKSRISITRDTSKNQFFLQL |
| 4691 | 15H6)HC | chain | NSVTPEDTATYYCTRGNWHYFDYWGQGTLVAVSSASTKGPSV |
| | (hole) | | FPLAPSSKSTSGGTAALGCLVKDYFPEPVTVSWNSGALTSGV |
| | | | HTFPAVLQSSGLYSLSSVVTVPSSSLGTQTYICNVNHKPSNT |
| | | | KVDKRVEPKSCDKTHTCPPCPAPELLGGPSVFLFPPKPKDTL |
| | | | MISRTPEVTCVVVDVSHEDPEVKFNWYVDGVEVHNAKTKPRE |
| | | | EQYNSTYRVVSVLTVLHQDWLNGKEYKCKVSNKALPAPIEKT |
| | | | ISKAKGQPREPQVCTLPPSREEMTKNQVSLSCAVKGFYPSDI |
| | | | AVEWESNGQPENNYKTTPPVLDSDGSFFLVSKLTVDKSRWQQ |
| | | | GNVFSCSVMHEALHNHYTQKSLSLSP |
| SEQ ID | Ch(anti- | 15H6 light | SYTLTQPPSLSVAPGQKATIICSGENLSDKYVHWYQQKPGRA |
| NO: | NKp30 | chain | PVMVIYENEKRPSGIPDQFSGSNSGNIATLTISKAQPGSEAD |
| 6153 | 15H6)LC | | YYCHYWESINSVVFGSGTHLTVLGQPKANPTVTLFPPSSEEL |
| | | | QANKATLVCLISDFYPGAVTVAWKADGSPVKAGVETTKPSKQ |
| | | | SNNKYAASSYLSLTPEQWKSHRSYSCQVTHEGSTVEKTVAPT |
| | | | ECS |
| SEQ ID | anti- | Hamster | QIQLQESGPGLVKPSQSLSLTCSVTGFSINTGGYHWNWIRQF |
| NO: | NKp30 | anti- | PGKKLEWMGYIYSSGSTSYNPSLKSRISITRDTSKNQFFLQL |
| 6187 | 9G1 scFv | NKp30 | NSVTTEDTATYYCARGNWHYFDFWGQGTMVTVSSGGGGSGGG |
| | (VH-VL) | scFv of | GSGGGGSGGGGSSYTLTQPPLLSVALGHKATITCSGERLSDK |
| | | 9G1 in VH | YVHWYQQKPGRAPVMVIYENDKRPSGIPDQFSGSNSGNIATL |
| | | toVL | TISKAQAGYEADYYCQSWDSTNSAVFGSGTQLTVL |
| | | orientation | |
| SEQ ID | anti- | Hamster | SYTLTQPPLLSVALGHKATITCSGERLSDKYVHWYQQKPGRA |
| NO: | NKp30 | anti- | PVMVIYENDKRPSGIPDQFSGSNSGNIATLTISKAQAGYEAD |
| 6188 | 9G1 scFv | NKp30 | YYCQSWDSTNSAVFGSGTQLTVLGGGGSGGGGSGGGGSGGGG |
| | (VL-VH) | scFv of | SQIQLQESGPGLVKPSQSLSLTCSVTGFSINTGGYHWNWIRQ |
| | | 9G1 in VL | FPGKKLEWMGYIYSSGSTSYNPSLKSRISITRDTSKNQFFLQ |
| | | toVH | LNSVTTEDTATYYCARGNWHYFDFWGQGTMVTVSS |
| | | orientation | |
| SEQ ID | anti- | Hamster | QIQLQESGPGLVKPSQSLSLTCSVTGFSINTGGYHWNWIRQF |
| NO: | NKp30 | anti- | PGKKLEWMGYIYSSGTTRYNPSLKSRISITRDTSKNQFFLQL |
| 6189 | 15H6 scFv | NKp30 | NSVTPEDTATYYCTRGNWHYFDYWGQGTLVAVSSGGGGSGGG |
| | (VH-VL) | scFv of | GSGGGGSGGGGSSYTLTQPPSLSVAPGQKATIICSGENLSDK |
| | | 15H6 in | YVHWYQQKPGRAPVMVIYENEKRPSGIPDQFSGSNSGNIATL |
| | | VH to VL | TISKAQPGSEADYYCHYWESINSVVFGSGTHLTVL |
| | | orientation | |
| SEQ ID | anti- | Hamster | SYTLTQPPSLSVAPGQKATIICSGENLSDKYVHWYQQKPGRA |
| NO: | NKp30 | anti- | PVMVIYENEKRPSGIPDQFSGSNSGNIATLTISKAQPGSEAD |
| 6190 | 15H6 scFv | NKp30 | YYCHYWESINSVVFGSGTHLTVLGGGGSGGGGSGGGGSGGGG |
| | (VL-VH) | scFv of | SQIQLQESGPGLVKPSQSLSLTCSVTGFSINTGGYHWNWIRQ |
| | | 15H6 in | FPGKKLEWMGYIYSSGTTRYNPSLKSRISITRDTSKNQFFLQ |
| | | VL to VH | LNSVTPEDTATYYCTRGNWHYFDYWGQGTLVAVSS |
| | | orientation | |
| SEQ ID | BJM0859 | | EIQLLESGGGLVQPGGSLRLSCAVSGFSITTTGYHWNWVRQA |
| NO: | lambda | | PGKGLEWVGYIYSSGSTSYNPSLKSRFTISRDTSKNTFYLQM |
| 7310 | scFv | | NSLRAEDTAVYYCARGDWHYFDYWGQGTMVTVSSGGGGSGGG |
| | | | GSGGGGSGGGGSSSETTQPPSVSVSPGQTASITCSGEKLSDK |
| | | | YVHWYQQKPGQSPVMVIYENDRRPSGIPERFSGSNSGNTATL |
| | | | TISGTQAMDEADYFCQFWDSTNSAVFGGGTQLTVL |
| SEQ ID | BJM0860 | | EIQLLESGGGLVQPGGSLRLSCAVSGFSITTTGYHWNWVRQA |
| NO: | kappa scFv | | PGKGLEWVGYIYSSGSTSYNPSLKSRFTISRDTSKNTFYLQM |
| 7311 | | | NSLRAEDTAVYYCARGDWHYFDYWGQGTMVTVSSGGGGSGGG |
| | | | GSGGGGSGGGGSDSVTTQSPLSLPVTLGQPASISCSGEKLSD |
| | | | KYVHWYQQRPGQSPRMLIYENDRRPSGVPDRFSGSNSGNDAT |
| | | | LKISRVEAEDVGVYFCQFWDSTNSAVFGGGTKVEIK |
| SEQ ID | BKM0138 | | EIQLLESGGGLVQPGGSLRLSCAVSGFSITTTGYHWNWVRQA |
| NO: | scFv | | PGKGLEWVGYIYSSGSTSYNPSLKSRFTISRDTSKNTFYLQM |
| 386 | | | NSLRAEDTAVYYCARGDWHYFDYWGQGTMVTVSSGGGGSGGG |
| | | | GSGGGGSGGGGSDSVTTQSPLSLPVTLGQPASISCSGEKLSD |
| | | | KYVHWYQQRPGQSPRMLIYENDRRPSGVPDRFSGSNSGNDAT |
| | | | LKISRVEAEDVGVYFCQFWDSTASAVFGGGTKVEIK |
| SEQ ID | BKM0139 | | EIQLLESGGGLVQPGGSLRLSCAVSGFSITTTGYHWNWVRQA |
| NO: | scFv | | PGKGLEWVGYIYSSGSTSYNPSLKSRFTISRDTSKNTFYLQM |
| 387 | | | NSLRAEDTAVYYCARGDWHYFDYWGQGTMVTVSSGGGGSGGG |
| | | | GSGGGGSGGGGSDSVTTQSPLSLPVTLGQPASISCSGEKLSD |
| | | | KYVHWYQQRPGQSPRMLIYENDRRPSGVPDRFSGSNSGNDAT |
| | | | LKISRVEAEDVGVYFCQFWASTNSAVFGGGTKVEIK |
| SEQ ID | BKM0140 | | EIQLLESGGGLVQPGGSLRLSCAVSGFSITTTGYHWNWVRQA |
| NO: | scFv | | PGKGLEWVGYIYSSGSTSYNPSLKSRFTISRDTSKNTFYLQM |
| 388 | | | NSLRAEDTAVYYCARGDWHYFDYWGQGTMVTVSSGGGGSGGG |
| | | | GSGGGGSGGGGSSSETTQPPSVSVSPGQTASITCSGEKLSDK |
| | | | YVHWYQQKPGQSPVMVIYENDRRPSGIPERFSGSNSGNTATL |
| | | | TISGTQAMDEADYFCQFWASTNSAVFGGGTQLTVL |
| SEQ ID | BKM0141 | | EIQLLESGGGLVQPGGSLRLSCAVSGFSITTTGYHWNWVRQA |
| NO: | scFv | | PGKGLEWVGYIYSSGSTSYNPSLKSRFTISRDTSKNTFYLQM |
| 389 | | | NSLRAEDTAVYYCARGDWHYFDYWGQGTMVTVSSGGGGSGGG |
| | | | GSGGGGSGGGGSSSETTQPPSVSVSPGQTASITCSGEKLSDK |
| | | | YVHWYQQKPGQSPVMVIYENDRRPSGIPERFSGSNSGNTATL |
| | | | TISGTQAMDEADYFCQFWDSTASAVFGGGTQLTVL |
| SEQ ID | BKM0142 | | EIQLLESGGGLVQPGGSLRLSCAVSGFSITTTGYHWNWVRQA |
| NO: | scFv | | PGKGLEWVGYIYSSGSTSYAPSLKSRFTISRDTSKNTFYLQM |
| 390 | | | NSLRAEDTAVYYCARGDWHYFDYWGQGTMVTVSSGGGGSGGG |
| | | | GSGGGGSGGGGSDSVTTQSPLSLPVTLGQPASISCSGEKLSD |
| | | | KYVHWYQQRPGQSPRMLIYENDRRPSGVPDRFSGSNSGNDAT |
| | | | LKISRVEAEDVGVYFCQFWDSTNSAVFGGGTKVEIK |
| SEQ ID | BKM0143 | | EIQLLESGGGLVQPGGSLRLSCAVSGFSITTTGYHWNWVRQA |
| NO: | scFv | | PGKGLEWVGYIYSSGSTSYAPSLKSRFTISRDTSKNTFYLQM |
| 391 | | | NSLRAEDTAVYYCARGDWHYFDYWGQGTMVTVSSGGGGSGGG |
| | | | GSGGGGSGGGGSSSETTQPPSVSVSPGQTASITCSGEKLSDK |
| | | | YVHWYQQKPGQSPVMVIYENDRRPSGIPERFSGSNSGNTATL |
| | | | TISGTQAMDEADYFCQFWDSTNSAVFGGGTQLTVL |
| SEQ ID | BKM0144 | | EIQLLESGGGLVQPGGSLRLSCAVSGFSITTTGYHWNWVRQA |
| NO: | scFv | | PGKGLEWVGYIYSSGSTSYAPSLKSRFTISRDTSKNTFYLQM |
| 392 | | | NSLRAEDTAVYYCARGDWHYFDYWGQGTMVTVSSGGGGSGGG |
| | | | GSGGGGSGGGGSDSVTTQSPLSLPVTLGQPASISCSGEKLSD |
| | | | KYVHWYQQRPGQSPRMLIYENDRRPSGVPDRFSGSNSGNDAT |
| | | | LKISRVEAEDVGVYFCQFWASTASAVFGGGTKVEIK |
| SEQ ID | BKM0145 | | EIQLLESGGGLVQPGGSLRLSCAVSGFSITTTGYHWNWVRQA |
| NO: | scFv | | PGKGLEWVGYIYSSGSTSYAPSLKSRFTISRDTSKNTFYLQM |
| 393 | | | NSLRAEDTAVYYCARGDWHYFDYWGQGTMVTVSSGGGGSGGG |
| | | | GSGGGGSGGGGSSSETTQPPSVSVSPGQTASITCSGEKLSDK |
| | | | YVHWYQQKPGQSPVMVIYENDRRPSGIPERFSGSNSGNTATL |
| | | | TISGTQAMDEADYFCQFWASTASAVFGGGTQLTVL |

TABLE 27
Exemplary variable regions of NKp46, NKG2D, or CD16-targeting antigen
binding domains

| SEQ ID NO | Ab ID | Description | Sequence |
|---|---|---|---|
| SEQ ID | NKG2D_1scFV | scFv that | QVHLQESGPGLVKPSETLSLTCTVSDDSISSYYWSWIRQPPG |
| NO: 6175 | | binds | KGLEWIGHISYSGSANYNPSLKSRVTISVDTSKNQFSLKLSSV |
| | | NKG2D | TAADTAVYYCANWDDAFNIWGQGTMVTVSSGGGGSGGGG |
| | | | SGGGGSGGGGSEIVLTQSPGTLSLSPGERATLSCRASQSVSSS |
| | | | YLAWYQQKPGQAPRLLIYGASSRATGIPDRFSGSGSGTDFTL |
| | | | TISRLEPEDFAVYYCQQYGSSPWTFGQGTKVEIK |
| SEQ ID | NKG2D_1VH | VH that binds | QVHLQESGPGLVKPSETLSLTCTVSDDSISSYYWSWIRQPPG |
| NO: 6176 | | NKG2D | KGLEWIGHISYSGSANYNPSLKSRVTISVDTSKNQFSLKLSSV |
| | | | TAADTAVYYCANWDDAFNIWGQGTMVTVSS |
| SEQ ID | NKG2D_1VL | VL that binds | EIVLTQSPGTLSLSPGERATLSCRASQSVSSSYLAWYQQKPG |
| NO: 6177 | | NKG2D | QAPRLLIYGASSRATGIPDRFSGSGSGTDFTLTISRLEPEDFAV |
| | | | YYCQQYGSSPWTFGQGTKVEIK |
| SEQ ID | NKG2D_2scFV | scFv that | EVQLVQSGAEVKEPGESLKISCKNSGYSFTNYWVGWVRQM |
| NO: 6178 | | binds | PGKGLEWMGIIYPGDSDTRYSPSFQGQVTISADKSINTAYLQ |
| | | NKG2D | WSSLKASDTAMYYCGRLTMFRGIIIGYFDYWGQGTLVTVSS |
| | | | GGGGSGGGGSGGGGGGGGSEIVLTQSPATLSLSPGERATLS |
| | | | CRASQSVSSYLAWYQQKPGQAPRLLIYDASNRATGIPARFSG |
| | | | SGSGTDFTLTISSLEPEDFAVYYCQQRSNWPWTFGQGTKVEIK |
| SEQ ID | NKG2D_2VH | VH that binds | EVQLVQSGAEVKEPGESLKISCKNSGYSFTNYWVGWVRQM |
| NO: 6179 | | NKG2D | PGKGLEWMGIIYPGDSDTRYSPSFQGQVTISADKSINTAYLQ |
| | | | WSSLKASDTAMYYCGRLTMFRGIIIGYFDYWGQGTLVTVSS |
| SEQ ID | NKG2D_2VL | VL that binds | EIVLTQSPATLSLSPGERATLSCRASQSVSSYLAWYQQKPGQ |
| NO: 6180 | | NKG2D | APRLLIYDASNRATGIPARFSGSGSGTDFTLTISSLEPEDFAVY |
| | | | YCQQRSNWPWTFGQGTKVEIK |
| SEQ ID | NKp46scFV | scFv that | QVQLQQSGPELVKPGASVKMSCKASGYTFTDYVINWGKQR |
| NO: 6181 | | binds NKp46 | SGQGLEWIGEIYPGSGTNYYNEKFKAKATLTADKSSNIAYM |
| | | | QLSSLTSEDSAVYFCARRGRYGLYAMDYWGQGTSVTVSSG |
| | | | GGGSGGGGSGGGGSGGGGSDIQMTQTTSSLSASLGDRVTISC |
| | | | RASQDISNYLNWYQQKPDGTVKLLIYYTSRLHSGVPSRFSGS |
| | | | GSGTDYSLTINNLEQEDIATYFCQQGNTRPWTFGGGTKLEIK |
| SEQ ID | NKp46VH | VH that binds | QVQLQQSGPELVKPGASVKMSCKASGYTFTDYVINWGKQR |
| NO: 6182 | | NKp46 | SGQGLEWIGEIYPGSGTNYYNEKFKAKATLTADKSSNIAYM |
| | | | QLSSLTSEDSAVYFCARRGRYGLYAMDYWGQGTSVTVSS |
| SEQ ID | NKp46VL | VL that binds | DIQMTQTTSSLSASLGDRVTISCRASQDISNYLNWYQQKPDG |
| NO: 6183 | | NKp46 | TVKLLIYYTSRLHSGVPSRFSGSGSGTDYSLTINNLEQEDIAT |
| | | | YFCQQGNTRPWTFGGGTKLEIK |
| SEQ ID | CD16scFV | scFv that | EVQLVESGGGVVRPGGSLRLSCAASGFTFDDYGMSWVRQA |
| NO: 6184 | | binds CD16 | PGKGLEWVSGINWNGGSTGYADSVKGRFTISRDNAKNSLYL |
| | | | QMNSLRAEDTAVYYCARGRSLLFDYWGQGTLVTVSRGGGG |
| | | | SGGGGSGGGGSSELTQDPAVSVALGQTVRITCQGDSLRSYY |
| | | | ASWYQQKPGQAPVLVIYGKNNRPSGIPDRFSGSSSGNTASLT |
| | | | ITGAQAEDEADYYCNSRDSSGNHVVFGGGTKLTVL |
| SEQ ID | CD16VH | VH that binds | EVQLVESGGGVVRPGGSLRLSCAASGFTFDDYGMSWVRQA |
| NO: 6185 | | CD16 | PGKGLEWVSGINWNGGSTGYADSVKGRFTISRDNAKNSLYL |
| | | | QMNSLRAEDTAVYYCARGRSLLFDYWGQGTLVTVSR |
| SEQ ID | CD16VL | VL that binds | SSELTQDPAVSVALGQTVRITCQGDSLRSYYASWYQQKPGQ |
| NO: 6186 | | CD16 | APVLVIYGKNNRPSGIPDRFSGSSSGNTASLTITGAQAEDEAD |
| | | | YYCNSRDSSGNHVVFGGGTKLTVL |

TABLE 29
List of TCRβV subfamilies and subfamily members

| Subfamily | Subfamily members |
|---|---|
| TCRβ V6 | TCRβ V6-4*01, TCRβ V6-4*02, TCRβ V6-9*01, |
| Also referred | TCRβ V6-8*01, TCRβ V6-5*01, TCRβ V6-6*02, |
| to as: | TCRβ V6-6*01, TCRβ V6-2*01, TCRβ V6-3*01 |
| TCR VB 13.1 | or TCRβ V6-1*01. |
| TCRβ V10 | TCRβ V10-1*01, TCRβ V10-1*02, |
| Also referred | TCRβ V10-3*01 or TCRβV10-2*01 |
| to as: | |
| TCRβ V12 | |
| TCRβ V12 | TCRβ V12-4*01, TCRβ V12-3*01, or |
| Also referred | TCRβ V12-5*01 |
| to as: | |
| TCRβ V8.1 | |
| TCRβ V5 | TCRβ V5-5*01, TCRβ V5-6*01, TCRβ V5-4*01, |
| | TCRβ V5-8*01, TCRβ V5-1*01 |
| TCRβ V7 | TCRβ V7-7*01, TCRβ V7-6*01, TCRβ V7-8*02, |
| | TCRβ V7-4*01, TCRβ V7-2*02, TCRβ V7-2*03, |
| | TCRβ V7-2*01, TCRβ V7-3*01, TCRβ V7-9*03, |
| | or TCRβ V7-9*01 |
| TCRβ V11 | TCRβ V11-1*01, TCRβ V11-2*01 or |
| | TCRβ V11-3*01 |
| TCRβ V14 | TCRβ V14*01 |
| TCRβ V16 | TCRβ V16*01 |
| TCRβ V18 | TCRβ V18*01 |
| TCRβ V9 | TCRβ V9*01 or TCRβ V9*02 |
| TCRβ V13 | TCRβ V13*01 |
| TCRβ V4 | TCRβ V4-2*01, TCRβ V4-3*01, or |
| | TCRβ V4-1*01 |
| TCRβ V3 | TCRβ V3-1*01 |
| TCRβ V2 | TCRβ V2*01 |
| TCRβ V15 | TCRβ V15*01 |
| TCRβ V30 | TCRβ V30*01, or TCRβ V30*02 |
| TCRβ V19 | TCRβ V19*01, or TCRβ V19*02 |
| TCRβ V27 | TCRβ V27*01. |
| TCRβ V28 | TCRβ V28*01. |
| TCRβ V24 | TCRβ V24-1*01 |
| TCRβ V20 | TCRβ V20-1*01, or TCRβ V20-1*02 |
| TCRβ V25 | TCRβ V25-1*01 |
| TCRβ V29 | TCRβ V29-1*01 |

TABLE 30
Amino acid and nucleotide sequences for murine, chimeric and humanized antibody
molecules which bind to TCRVB 6, e.g., TCRVB 6-5. The antibody molecules include murine
Antibody A, and humanized mAb Antibody A-H Clones A-H.1 to A-H.85. The amino acid the
mAb heavy and light chain CDRs, and the amino acid and nucleotide sequences of the heavy
and light chain variable regions, and the heavy and light chains are shown.
also referred to as H131, TCRVB 6-5 binder
Antibody A (murine), also referred to as H131, TCRVB 6-5 binder

| | | |
|---|---|---|
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 45 | HC CDR1 (Kabat) | TYYIH |
| SEQ ID NO: 46 | HC CDR2 (Kabat) | WFFPGSGNIKYNEKFKG |
| SEQ ID NO: 47 | HC CDR3 (Kabat) | SYYSYDVLDY |
| SEQ ID NO: 48 | HC CDR1 (Chothia) | GYSFTTY |
| SEQ ID NO: 49 | HC CDR2 (Chothia) | FPGSGN |
| SEQ ID NO: 50 | HC CDR3 (Chothia) | SYYSYDVLDY |
| SEQ ID NO: 1 | VH | QVQLQQSGPELVKPGTSVKISCKASGYSFTTYYIHWVKQRPG |
| | | QGLEWIGWFFPGSGNIKYNEKFKGKATLTADTSSSTAYMQLS |
| | | SLTSEESAVYFCAGSYYSYDVLDYWGHGTTLTVSS |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 51 | LC CDR1 (Kabat) | KASQNVGINVV |
| SEQ ID NO: 52 | LC CDR2 (Kabat) | SSSHRYS |
| SEQ ID NO: 53 | LC CDR3 (Kabat) | QQFKSYPLT |
| SEQ ID NO: 54 | LC CDR1 (Chothia) | KASQNVGINVV |
| SEQ ID NO: 55 | LC CDR2 (Chothia) | SSSHRYS |
| SEQ ID NO: 56 | LC CDR3 (Chothia) | QQFKSYPLT |
| SEQ ID NO: 2 | VL | DILMTQSQKFMSTSLGDRVSVSCKASQNVGINVVWHQQKPG |
| | | QSPKALIYSSSHRYSGVPDRFTGSGSGTDFTLTINNVQSEDLA |
| | | EYFCQQFKSYPLTFGAGTKLELK |

Antibody A humanized (A-H antibody), TCRVB 6-5 binder
A-H.1 antibody (also referred to as BHM1709)

| | | |
|---|---|---|
| SEQ ID NO: 3 | HC CDR1 (Combined) | GYSFTTYYIH |
| SEQ ID NO: 4 | HC CDR2 (Combined) | WFFPGSGNIK YNEKFKG |
| SEQ ID NO: 5 | HC CDR3 (Combined) | SYYSYDVLDY |
| SEQ ID NO: 9 | VH | QVQLVQSGAEVKKPGSSVKVSCKASGYSFTTYYIHWVRQAP |
| | | GQGLEWMGWFFPGSGNIKYNEKFKGRVTITADTSTSTAYME |
| | | LSSLRSEDTAVYYCAGSYYSYDVLDYWGQGTTVTVSS |
| SEQ ID NO: 12 | DNA VH | CAGGTGCAGCTGGTTCAGTCTGGCGCCGAAGTGAAGAAAC |
| | | CTGGCTCCTCCGTGAAGGTGTCCTGCAAGGCTTCCGGCTAC |
| | | TCCTTCACCACCTACTACATCCACTGGGTCCGACAGGCCCC |
| | | TGGACAAGGATTGGAATGGATGGGCTGGTTCTTCCCCGGC |
| | | TCCGGCAACATCAAGTACAACGAGAAGTTCAAGGGCCGCG |
| | | TGACCATCACCGCCGACACCTCTACCTCTACCGCCTACATG |
| | | GAACTGTCCAGCCTGAGATCTGAGGACACCGCCGTGTACT |
| | | ACTGCGCCGGCTCCTACTACTCTTACGACGTGCTGGATTAC |
| | | TGGGGCCAGGGCACCACAGTGACAGTGTCCTCT |
| SEQ ID NO: 69 | VH-IgM constant delta | METDTLLLWVLLLWVPGSTGQVQLVQSGAEVKKPGSSVKVS |
| | CDC | CKASGYSFTTYYIHWVRQAPGQGLEWMGWFFPGSGNIKYNE |
| | | KFKGRVTITADTSTSTAYMELSSLRSEDTAVYYCAGSYYSYD |
| | | VLDYWGQGTTVTVSSGSASAPTLFPLVSCENSPSDTSSVAVG |
| | | CLAQDFLPDSITFSWKYKNNSDISSTRGFPSVLRGGKYAATSQ |
| | | VLLPSKDVMQGTDEHVVCKVQHPNGNKEKNVPLPVIAELPP |
| | | KVSVFVPPRDGFFGNPRKSKLICQATGFSPRQIQVSWLREGKQ |
| | | VGSGVTTDQVQAEAKESGPTTYKVTSTLTIKESDWLGQSMFT |
| | | CRVDHRGLTFQQNASSMCVPDQDTAIRVFAIPPSFASIFLTKS |
| | | TKLTCLVTDLTTYDSVTISWTRQNGEAVKTHTNISESHPNATF |
| | | SAVGEASICEDDWNSGERFTCTVTHTDLASSLKQTISRPKGVA |
| | | LHRPDVYLLPPAREQLNLRESATITCLVTGFSPADVFVQWMQ |
| | | RGQPLSPEKYVTSAPMPEPQAPGRYFAHSILTVSEEEWNTGET |
| | | YTCVVAHEALPNRVTERTVDKSTGKPTLYNVSLVMSDTAGT |
| | | CY |
| SEQ ID NO: 70 | VH-IgGA1 | METDTLLLWVLLLWVPGSTGQVQLVQSGAEVKKPGSSVKVS |
| | | CKASGYSFTTYYIHWVRQAPGQGLEWMGWFFPGSGNIK YNE |
| | | KFKGRVTITADTSTSTAYMELSSLRSEDTAVYYCAGSYYSYD |
| | | VLDYWGQGTTVTVSSASPTSPKVFPLSLCSTQPDGNVVIACL |
| | | VQGFFPQEPLSVTWSESGQGVTARNFPPSQDASGDLYTTSSQ |
| | | LTLPATQCLAGKSVTCHVKHYTNPSQDVTVPCPVPSTPPTPSP |
| | | STPPTPSPSCCHPRLSLHRPALEDLLLGSEANLTCTLTGLRDAS |
| | | GVTFTWTPSSGKSAVQGPPERDLCGCYSVSSVLPGCAEPWNH |
| | | GKTFTCTAAYPESKTPLTATLSKSGNTFRPEVHLLPPPSEELAL |
| | | NELVTLTCLARGFSPKDVLVRWLQGSQELPREKYLTWASRQ |
| | | EPSQGTTTFAVTSILRVAAEDWKKGDTFSCMVGHEALPLAFT |
| | | QKTIDRLAGKPTHVNVSVVMAEVDGTCY |
| SEQ ID NO: 71 | VH-IgGA2 | METDTLLLWVLLLWVPGSTGQVQLVQSGAEVKKPGSSVKVS |
| | | CKASGYSFTTYYIHWVRQAPGQGLEWMGWFFPGSGNIKYNE |
| | | KFKGRVTITADTSTSTAYMELSSLRSEDTAVYYCAGSYYSYD |
| | | VLDYWGQGTTVTVSSASPTSPKVFPLSLDSTPQDGNVVVACL |
| | | VQGFFPQEPLSVTWSESGQNVTARNFPPSQDASGDLYTTSSQ |
| | | LTLPATQCPDGKSVTCHVKHYTNSSQDVTVPCRVPPPPPCCH |
| | | PRLSLHRPALEDLLLGSEANLTCTLTGLRDASGATFTWTPSSG |
| | | KSAVQGPPERDLCGCYSVSSVLPGCAQPWNHGETFTCTAAH |
| | | PELKTPLTANITKSGNTFRPEVHLLPPPSEELALNELVTLTCLA |
| | | RGFSPKDVLVRWLQGSQELPREKYLTWASRQEPSQGTTTYA |
| | | VTSILRVAAEDWKKGETFSCMVGHEALPLAFTQKTIDRMAG |
| | | KPTHINVSVVMAEADGTCY |
| SEQ ID NO: | Heavy chain | METDTLLLWVLLLWVPGSTGQVQLVQSGAEVKKPGSSVKVS |
| 3278 | | CKASGYSFTTYYIHWVRQAPGQGLEWMGWFFPGSGNIKYNE |
| | | KFKGRVTITADTSTSTAYMELSSLRSEDTAVYYCAGSYYSYD |
| | | VLDYWGQGTTVTVSSASTKGPSVFPLAPSSKSTSGGTAALGC |
| | | LVKDYFPEPVTVSWNSGALTSGVHTFPAVLQSSGLYSLSSVV |
| | | TVPSSSLGTQTYICNVNHKPSNTKVDKRVEPKSCDKTHTCPP |
| | | CPAPELLGGPSVFLFPPKPKDTLMISRTPEVTCVVVDVSHEDP |
| | | EVKFNWYVDGVEVHNAKTKPREEQYNSTYRVVSVLTVLHQ |
| | | DWLNGKEYKCKVSNKALPAPIEKTISKAKGQPREPQVYTLPP |
| | | SREEMTKNQVSLTCLVKGFYPSDIAVEWESNGQPENNYKTTP |
| | | PVLDSDGSFFLYSKLTVDKSRWQQGNVFSCSVMHEALHNHY |
| | | TQKSLSLSPGK |
| SEQ ID NO: | Heavy chain | METDTLLLWVLLLWVPGSTGQVQLVQSGAEVKKPGSSVKVS |
| 4692 | | CKASGYSFTTYYIHWVRQAPGQGLEWMGWFFPGSGNIKYNE |
| | | KFKGRVTITADTSTSTAYMELSSLRSEDTAVYYCAGSYYSYD |
| | | VLDYWGQGTTVTVSSASTKGPSVFPLAPSSKSTSGGTAALGC |
| | | LVKDYFPEPVTVSWNSGALTSGVHTFPAVLQSSGLYSLSSVV |
| | | TVPSSSLGTQTYICNVNHKPSNTKVDKRVEPKSCDKTHTCPP |
| | | CPAPELLGGPSVFLFPPKPKDTLMISRTPEVTCVVVDVSHEDP |
| | | EVKFNWYVDGVEVHNAKTKPREEQYNSTYRVVSVLTVLHQ |
| | | DWLNGKEYKCKVSNKALPAPIEKTISKAKGQPREPQVYTLPP |
| | | SREEMTKNQVSLTCLVKGFYPSDIAVEWESNGQPENNYKTTP |
| | | PVLDSDGSFFLYSKLTVDKSRWQQGNVFSCSVMHEALHNHY |
| | | TQKSLSLSP |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 10 | VL | DIQMTQSPSFLSASVGDRVTITCKASQNVGINVVWHQQKPGK |
| | | APKALIYSSSHRYSGVPSRFSGSGSGTEFTLTISSLQPEDFATYF |
| | | CQQFKSYPLTFGQGTKLEIK |
| SEQ ID NO: | DNA VL | GACATCCAGATGACCCAGTCTCCATCCTTCCTGTCCGCCTC |
| 383 | | TGTGGGCGACAGAGTGACCATCACATGCAAGGCCTCTCAG |
| | | AACGTGGGCATCAACGTCGTGTGGCACCAGCAGAAGCCTG |
| | | GCAAGGCTCCTAAGGCTCTGATCTACTCCTCCAGCCACCG |
| | | GTACTCTGGCGTGCCCTCTAGATTTTCCGGCTCTGGCTCTG |
| | | GCACCGAGTTTACCCTGACAATCTCCAGCCTGCAGCCTGA |
| | | GGACTTCGCCACCTACTTTTGCCAGCAGTTCAAGAGCTACC |
| | | CTCTGACCTTTGGCCAGGGCACCAAGCTGGAAATCAAG |
| SEQ ID NO: 72 | VL and kappa constant | METDTLLLWVLLLWVPGSTGDIQMTQSPSFLSASVGDRVTIT |
| | region/light chain | CKASQNVGINVVWHQQKPGKAPKALIYSSSHRYSGVPSRFSG |
| | | SGSGTEFTLTISSLQPEDFATYFCQQFKSYPLTFGQGTKLEIKR |
| | | TVAAPSVFIFPPSDEQLKSGTASVVCLLNNFYPREAKVQWKV |
| | | DNALQSGNSQESVTEQDSKDSTYSLSSTLTLSKADYEKHKVY |
| | | ACEVTHQGLSSPVTKSFNRGEC |

A-H.2 antibody (also referred to as BHM1710)

| | | |
|---|---|---|
| SEQ ID NO: 3 | HC CDR1 (Combined) | GYSFTTYYIH |
| SEQ ID NO: 4 | HC CDR2 (Combined) | WFFPGSGNIKYNEKFKG |
| SEQ ID NO: 5 | HC CDR3 (Combined) | SYYSYDVLDY |
| SEQ ID NO: 9 | VH | QVQLVQSGAEVKKPGSSVKVSCKASGYSFTTYYIHWVRQAP |
| | | GQGLEWMGWFFPGSGNIKYNEKFKGRVTITADTSTSTAYME |
| | | LSSLRSEDTAVYYCAGSYYSYDVLDYWGQGTTVTVSS |
| SEQ ID NO: 12 | DNA VH | CAGGTGCAGCTGGTTCAGTCTGGCGCCGAAGTGAAGAAAC |
| | | CTGGCTCCTCCGTGAAGGTGTCCTGCAAGGCTTCCGGCTAC |
| | | TCCTTCACCACCTACTACATCCACTGGGTCCGACAGGCCCC |
| | | TGGACAAGGATTGGAATGGATGGGCTGGTTCTTCCCCGGC |
| | | TCCGGCAACATCAAGTACAACGAGAAGTTCAAGGGCCGCG |
| | | TGACCATCACCGCCGACACCTCTACCTCTACCGCCTACATG |
| | | GAACTGTCCAGCCTGAGATCTGAGGACACCGCCGTGTACT |
| | | ACTGCGCCGGCTCCTACTACTCTTACGACGTGCTGGATTAC |
| | | TGGGGCCAGGGCACCACAGTGACAGTGTCCTCT |
| SEQ ID NO: | Heavy chain | METDTLLLWVLLLWVPGSTGQVQLVQSGAEVKKPGSSVKVS |
| 3278 | | CKASGYSFTTYYIHWVRQAPGQGLEWMGWFFPGSGNIKYNE |
| | | KFKGRVTITADTSTSTAYMELSSLRSEDTAVYYCAGSYYSYD |
| | | VLDYWGQGTTVTVSSASTKGPSVFPLAPSSKSTSGGTAALGC |
| | | LVKDYFPEPVTVSWNSGALTSGVHTFPAVLQSSGLYSLSSVV |
| | | TVPSSSLGTQTYICNVNHKPSNTKVDKRVEPKSCDKTHTCPP |
| | | CPAPELLGGPSVFLFPPKPKDTLMISRTPEVTCVVVDVSHEDP |
| | | EVKFNWYVDGVEVHNAKTKPREEQYNSTYRVVSVLTVLHQ |
| | | DWLNGKEYKCKVSNKALPAPIEKTISKAKGQPREPQVYTLPP |
| | | SREEMTKNQVSLTCLVKGFYPSDIAVEWESNGQPENNYKTTP |
| | | PVLDSDGSFFLYSKLTVDKSRWQQGNVFSCSVMHEALHNHY |
| | | TQKSLSLSPGK |
| SEQ ID NO: | Heavy chain | METDTLLLWVLLLWVPGSTGQVQLVQSGAEVKKPGSSVKVS |
| 4692 | | CKASGYSFTTYYIHWVRQAPGQGLEWMGWFFPGSGNIKYNE |
| | | KFKGRVTITADTSTSTAYMELSSLRSEDTAVYYCAGSYYSYD |
| | | VLDYWGQGTTVTVSSASTKGPSVFPLAPSSKSTSGGTAALGC |
| | | LVKDYFPEPVTVSWNSGALTSGVHTFPAVLQSSGLYSLSSVV |
| | | TVPSSSLGTQTYICNVNHKPSNTKVDKRVEPKSCDKTHTCPP |
| | | CPAPELLGGPSVFLFPPKPKDTLMISRTPEVTCVVVDVSHEDP |
| | | EVKFNWYVDGVEVHNAKTKPREEQYNSTYRVVSVLTVLHQ |
| | | DWLNGKEYKCKVSNKALPAPIEKTISKAKGQPREPQVYTLPP |
| | | SREEMTKNQVSLTCLVKGFYPSDIAVEWESNGQPENNYKTTP |
| | | PVLDSDGSFFLYSKLTVDKSRWQQGNVFSCSVMHEALHNHY |
| | | TQKSLSLSP |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 11 | VL | DIQMTQSPSSLSASVGDRVTITCKASQNVGINVVWHQQKPGK |
| | | VPKALIYSSSHRYSGVPSRFSGSGSGTDFTLTISSLQPEDVATY |
| | | FCQQFKSYPLTFGQGTKLEIK |
| SEQ ID NO: 14 | DNA VL | GACATCCAGATGACCCAGTCTCCATCCTCTCTGTCCGCCTC |
| | | TGTGGGCGACAGAGTGACCATCACATGCAAGGCCTCTCAG |
| | | AACGTGGGCATCAACGTCGTGTGGCACCAGCAGAAACCTG |
| | | GCAAGGTGCCCAAGGCTCTGATCTACTCCTCCAGCCACAG |
| | | ATACTCCGGCGTGCCCTCTAGATTCTCCGGCTCTGGCTCTG |
| | | GCACCGACTTTACCCTGACAATCTCCAGCCTGCAGCCTGA |
| | | GGACGTGGCCACCTACTTTTGCCAGCAGTTCAAGAGCTAC |
| | | CCTCTGACCTTTGGCCAGGGCACCAAGCTGGAAATCAAG |
| SEQ ID NO: | Light chain | METDTLLLWVLLLWVPGSTGDIQMTQSPSSLSASVGDRVTIT |
| 3279 | | CKASQNVGINVVWHQQKPGKVPKALIYSSSHRYSGVPSRFSG |
| | | SGSGTDFTLTISSLQPEDVATYFCQQFKSYPLTFGQGTKLEIKR |
| | | TVAAPSVFIFPPSDEQLKSGTASVVCLLNNFYPREAKVQWKV |
| | | DNALQSGNSQESVTEQDSKDSTYSLSSTLTLSKADYEKHKVY |
| | | ACEVTHQGLSSPVTKSFNRGEC |

A-H.3 antibody

| | | |
|---|---|---|
| SEQ ID NO: 80 | VH + VL | QVQLVQSGAEVKKPGSSVKVSCKASGTDFKLTYIHWVRQAP |
| | | GQGLEWMGRVSPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: 81 | VL | DIQMTQSPSFLSASVGDRVTITCKASQNVEDRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 82 | VH | QVQLVQSGAEVKKPGSSVKVSCKASGTDFKLTYIHWVRQAP |
| | | GQGLEWMGRVSPGSGNTKYNEKFKGRVTITADTSTSTAYME |
| | | LSSLRSEDTAVYYCAGSYYSYDVLDYWGQGTTVTVSS |

A-H.4

| | | |
|---|---|---|
| SEQ ID NO: 83 | VH + VL | QVQLVQSGAEVKKPGSSVKVSCKASGTDFDKIYIHWVRQAP |
| | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: 84 | VL | DIQMTQSPSFLSASVGDRVTITCKASQNVEDRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 85 | VH | QVQLVQSGAEVKKPGSSVKVSCKASGTDFDKIYIHWVRQAP |
| | | GQGLEWMGRISAGSGNVKYNEKFKGRVTITADTSTSTAYME |
| | | LSSLRSEDTAVYYCAGSYYSYDVLDYWGQGTTVTVSS |

A-H.5

| | | |
|---|---|---|
| SEQ ID NO: | VH + VL | QVQLVQSGAEVKKPGSSVKVSCKASGHDFRDFYIHWVRQAP |
| 6006 | | GQGLEWMGRVYPGSGSYRYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: 87 | VL | DIQMTQSPSFLSASVGDRVTITCKASQNVDDRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 88 | VH | QVQLVQSGAEVKKPGSSVKVSCKASGHDFRDFYIHWVRQAP |
| | | GQGLEWMGRVYPGSGSYRYNEKFKGRVTITADTSTSTAYME |
| | | LSSLRSEDTAVYYCAGSYYSYDVLDYWGQGTTVTVSS |

A-H.6

| | | |
|---|---|---|
| SEQ ID NO: 89 | VH + VL | QVQLVQSGAEVKKPGSSVKVSCKASGHDFKLTYIHWVRQAP |
| | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: 90 | VL | DIQMTQSPSFLSASVGDRVTITCKASQNVDNRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 91 | VH | QVQLVQSGAEVKKPGSSVKVSCKASGHDFKLTYIHWVRQAP |
| | | GQGLEWMGRISAGSGNVKYNEKFKGRVTITADTSTSTAYME |
| | | LSSLRSEDTAVYYCAGSYYSYDVLDYWGQGTTVTVSS |

A-H.7

| | | |
|---|---|---|
| SEQ ID NO: 92 | VH + VL | QVQLVQSGAEVKKPGSSVKVSCKASGTDFKLTYIHWVRQAP |
| | | GQGLEWMGRIFPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVENKVAWHQQKPGKAPKALIYSSSHRYKGVPSRFSGSGS |
| | | GTEFTLTISSLQPEDFATYFCQQFKSYPLTFGQGTKLEIK |
| SEQ ID NO: 93 | VL | DIQMTQSPSFLSASVGDRVTITCKASQNVENKVAWHQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 94 | VH | QVQLVQSGAEVKKPGSSVKVSCKASGTDFKLTYIHWVRQAP |
| | | GQGLEWMGRIFPGSGNVKYNEKFKGRVTITADTSTSTAYME |
| | | LSSLRSEDTAVYYCAGSYYSYDVLDYWGQGTTVTVSS |

A-H.8

| | | |
|---|---|---|
| SEQ ID NO: 95 | VH + VL | QVQLVQSGAEVKKPGSSVKVSCKASGTDFDKIYIHWVRQAP |
| | | GQGLEWMGRIFAGSGNTKYNEKFKGRVTITADTSTSTAYME |
| | | LSSLRSEDTAVYYCAGSYYSYDVLDYWGQGTTVTVSSGGGG |
| | | SGGGGSGGGGGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: 96 | VL | DIQMTQSPSFLSASVGDRVTITCKASQNVDDRVAWYQQKPG |
| | | KAPKALIYSSSHRYKGVPSRFSGSGSGTEFTLTISSLQPEDFAT |
| | | YFCQQFKSYPLTFGQGTKLEIK |
| SEQ ID NO: 97 | VH | QVQLVQSGAEVKKPGSSVKVSCKASGTDFDKIYIHWVRQAP |
| | | GQGLEWMGRIFAGSGNTKYNEKFKGRVTITADTSTSTAYME |
| | | LSSLRSEDTAVYYCAGSYYSYDVLDYWGQGTTVTVSS |

A-H.9

| | | |
|---|---|---|
| SEQ ID NO: 98 | VH + VL | QVQLVQSGAEVKKPGSSVKVSCKASGHDFDKFYIHWVRQAP |
| | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGNRVAWYQQKPGKAPKALIYSSSHRYSGVPSRFSGSGS |
| | | GTEFTLTISSLQPEDFATYFCQQFKSYPLTFGQGTKLEIK |
| SEQ ID NO: 99 | VL | DIQMTQSPSFLSASVGDRVTITCKASQNVGNRVAWYQQKPG |
| | | KAPKALIYSSSHRYSGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKFYIHWVRQAP |
| 100 | | GQGLEWMGRVSAGSGNVKYNEKFKGRVTITADTSTSTAYME |
| | | LSSLRSEDTAVYYCAGSYYSYDVLDYWGQGTTVTVSS |

A-H.10

| | | |
|---|---|---|
| SEQ ID NO: | VH + VL | QVQLVQSGAEVKKPGSSVKVSCKASGHDFDKFYIHWVRQAP |
| 101 | | GQGLEWMGRIFAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGDRVAWYQQKPG |
| 102 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKFYIHWVRQAP |
| 103 | | GQGLEWMGRIFAGSGNVKYNEKFKGRVTITADTSTSTAYME |
| | | LSSLRSEDTAVYYCAGSYYSYDVLDYWGQGTTVTVSS |

A-H.11

| | | |
|---|---|---|
| SEQ ID NO: | VH + VL | QVQLVQSGAEVKKPGSSVKVSCKASGTDFKLTYIHWVRQAP |
| 104 | | GQGLEWMGRVSPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGDRVAWYQQKPG |
| 105 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 106 | | GQGLEWMGRVSPGSGNVKYNEKFKGRVTITADTSTSTAYME |
| | | LSSLRSEDTAVYYCAGSYYSYDVLDYWGQGTTVTVSS |

A-H.12

| | | |
|---|---|---|
| SEQ ID NO: | VH + VL | QVQLVQSGAEVKKPGSSVKVSCKASGTDFDKIYIHWVRQAP |
| 107 | | GQGLEWMGRVSAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGNRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGNRVAWYQQKPG |
| 108 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 109 | | GQGLEWMGRVSAGSGNTKYNEKFKGRVTITADTSTSTAYME |
| | | LSSLRSEDTAVYYCAGSYYSYDVLDYWGQGTTVTVSS |

A-H.13, also referred to as A-H.69

| | | |
|---|---|---|
| SEQ ID NO: | VH + VL | QVQLVQSGAEVKKPGSSVKVSCKASGTDFKLTYIHWVRQAP |
| 110 | | GQGLEWMGRIFPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDNRVAWYQQKPG |
| 111 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 112 | | GQGLEWMGRIFPGSGNVKYNEKFKGRVTITADTSTSTAYME |
| | | LSSLRSEDTAVYYCAGSYYSYDVLDYWGQGTTVTVSS |

A-H.14

| | | |
|---|---|---|
| SEQ ID NO: | VH + VL | QVQLVQSGAEVKKPGSSVKVSCKASGTDFDKIYIHWVRQAP |
| 113 | | GQGLEWMGRISAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 114 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 115 | | GQGLEWMGRISAGSGNTKYNEKFKGRVTITADTSTSTAYME |
| | | LSSLRSEDTAVYYCAGSYYSYDVLDYWGQGTTVTVSS |

A-H.15

| | | |
|---|---|---|
| SEQ ID NO: | VH + VL | QVQLVQSGAEVKKPGSSVKVSCKASGTDFRLTYIHWVRQAP |
| 116 | | GQGLEWMGRVSPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNKVAWHQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDNKVAWHQQKPG |
| 117 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFRLTYIHWVRQAP |
| 118 | | GQGLEWMGRVSPGSGNTKYNEKFKGRVTITADTSTSTAYME |
| | | LSSLRSEDTAVYYCAGSYYSYDVLDYWGQGTTVTVSS |

A-H.16

| | | |
|---|---|---|
| SEQ ID NO: | VH + VL | QVQLVQSGAEVKKPGSSVKVSCKASGGTFRLTYIHWVRQAP |
| 119 | | GQGLEWMGRVYPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 120 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGGTFRLTYIHWVRQAP |
| 121 | | GQGLEWMGRVYPGSGNTKYNEKFKGRVTITADTSTSTAYME |
| | | LSSLRSEDTAVYYCAGSYYSYDVLDYWGQGTTVTVSS |

A-H.17

| | | |
|---|---|---|
| SEQ ID NO: | VH + VL | QVQLVQSGAEVKKPGSSVKVSCKASGTDFRLTYIHWVRQAP |
| 122 | | GQGLEWMGRIFPGSGNTKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVDDRVAWYQQKPGKAPKALIYSSSHRYKGVPSRFSGSGSG |
| | | TEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 123 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFRLTYIHWVRQAP |
| 124 | | GQGLEWMGRIFPGSGNTKYNEKFKGRVTITADTSTSTAYMEL |
| | | SSLRSEDTAVYYCAGSYYSYDVLDYWGQGTTVTVSS |

A-H.18

| | | |
|---|---|---|
| SEQ ID NO: | VH + VL | QVQLVQSGAEVKKPGSSVKVSCKASGTDFKLTYIHWVRQAP |
| 125 | | GQGLEWMGRIFPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVEDRVAWYQQKPG |
| 126 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 127 | | GQGLEWMGRIFPGSGNVKYNEKFKGRVTITADTSTSTAYME |
| | | LSSLRSEDTAVYYCAGSYYSYDVLDYWGQGTTVTVSS |

A-H.19

| | | |
|---|---|---|
| SEQ ID NO: | VH + VL | QVQLVQSGAEVKKPGSSVKVSCKASGGTFRLTYIHWVRQAP |
| 128 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGDRVAWYQQKPG |
| 129 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGGTFRLTYIHWVRQAP |
| 130 | | GQGLEWMGRISAGSGNVKYNEKFKGRVTITADTSTSTAYME |
| | | LSSLRSEDTAVYYCAGSYYSYDVLDYWGQGTTVTVSS |

A-H.20

| | | |
|---|---|---|
| SEQ ID NO: | VH + VL | QVQLVQSGAEVKKPGSSVKVSCKASGGTFDKTYIHWVRQAP |
| 131 | | GQGLEWMGRISAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 132 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGGTFDKTYIHWVRQAP |
| 133 | | GQGLEWMGRISAGSGNTKYNEKFKGRVTITADTSTSTAYME |
| | | LSSLRSEDTAVYYCAGSYYSYDVLDYWGQGTTVTVSS |

A-H.21

| | | |
|---|---|---|
| SEQ ID NO: | VH + VL | QVQLVQSGAEVKKPGSSVKVSCKASGHDFDKFYIHWVRQAP |
| 134 | | GQGLEWMGRISAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 135 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKFYIHWVRQAP |
| 136 | | GQGLEWMGRISAGSGNTKYNEKFKGRVTITADTSTSTAYME |
| | | LSSLRSEDTAVYYCAGSYYSYDVLDYWGQGTTVTVSS |

A-H.22

| | | |
|---|---|---|
| SEQ ID NO: | VH + VL | QVQLVQSGAEVKKPGSSVKVSCKASGTDFKLTYIHWVRQAP |
| 137 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNKVAWHQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDNKVAWHQQKPG |
| 138 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 139 | | GQGLEWMGRISAGSGNVKYNEKFKGRVTITADTSTSTAYME |
| | | LSSLRSEDTAVYYCAGSYYSYDVLDYWGQGTTVTVSS |

A-H.23

| | | |
|---|---|---|
| SEQ ID NO: | VH + VL | QVQLVQSGAEVKKPGSSVKVSCKASGHDFRLTYIHWVRQAP |
| 140 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVADRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVADRVAWYQQKPG |
| 141 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFRLTYIHWVRQAP |
| 142 | | GQGLEWMGRISAGSGNVKYNEKFKGRVTITADTSTSTAYME |
| | | LSSLRSEDTAVYYCAGSYYSYDVLDYWGQGTTVTVSS |

A-H.24

| | | |
|---|---|---|
| SEQ ID NO: | VH + VL | QVQLVQSGAEVKKPGSSVKVSCKASGHDFHLWYIHWVRQA |
| 143 | | PGQGLEWMGRVSAGSGNVKYNEKFKGRVTITADTSTSTAYM |
| | | ELSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVDNKVAWHQQKPGKAPKALIYSSSHRYKGVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVDNKVAWHQQKPG |
| 144 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFHLWYIHWVRQA |
| 145 | | PGQGLEWMGRVSAGSGNVKYNEKFKGRVTITADTSTSTAYM |
| | | ELSSLRSEDTAVYYCAGSYYSYDVLDYWGQGTTVTVSS |

A-H.25

| | | |
|---|---|---|
| SEQ ID NO: | VH + VL | QVQLVQSGAEVKKPGSSVKVSCKASGHDFHLWYIHWVRQA |
| 146 | | PGQGLEWMGRVFAGSGNTKYNEKFKGRVTITADTSTSTAYM |
| | | ELSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVEDKVAWYQQKPGKAPKALIYSSSHRYKGVPSRFSGSG |
| | | SGTEFTLTISSLQPEDFATYFCQQFKSYPLTFGQGTKLEIK |
| SEQ ID NO: | VL | DIQMTQSPSFLSASVGDRVTITCKASQNVEDKVAWYQQKPG |
| 147 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFHL WYIHWVRQA |
| 148 | | PGQGLEWMGRVFAGSGNTKYNEKFKGRVTITADTSTSTAYM |
| | | ELSSLRSEDTAVYYCAGSYYSYDVLDYWGQGTTVTVSS |

A-H.26

| | | |
|---|---|---|
| SEQ ID NO: | VH + VL | QVQLVQSGAEVKKPGSSVKVSCKASGTDFKLTYIHWVRQAP |
| 149 | | GQGLEWMGRIFPGSGNTKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVDDRVAWYQQKPGKAPKALIYSSSHRYKGVPSRFSGSGSG |
| | | TEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 150 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 151 | | GQGLEWMGRIFPGSGNTKYNEKFKGRVTITADTSTSTAYMEL |
| | | SSLRSEDTAVYYCAGSYYSYDVLDYWGQGTTVTVSS |

A-H.27

| | | |
|---|---|---|
| SEQ ID NO: | VH + VL | QVQLVQSGAEVKKPGSSVKVSCKASGTDFKLTYIHWVRQAP |
| 153 | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGNRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGNRVAWYQQKPG |
| 154 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 155 | | GQGLEWMGRVSAGSGNVKYNEKFKGRVTITADTSTSTAYME |
| | | LSSLRSEDTAVYYCAGSYYSYDVLDYWGQGTTVTVSS |

A-H.28

| | | |
|---|---|---|
| SEQ ID NO: | VH + VL | QVQLVQSGAEVKKPGSSVKVSCKASGTDFKLTYIHWVRQAP |
| 156 | | GQGLEWMGRISPGSGNTKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVGDRVAWYQQKPGKAPKALIYSSSHRYKGVPSRFSGSGSG |
| | | TEFTLTISSLQPEDFATYFCQQFKSYPLTFGQGTKLEIK |
| SEQ ID NO: | VL | DIQMTQSPSFLSASVGDRVTITCKASQNVGDRVAWYQQKPG |
| 157 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 158 | | GQGLEWMGRISPGSGNTKYNEKFKGRVTITADTSTSTAYMEL |
| | | SSLRSEDTAVYYCAGSYYSYDVLDYWGQGTTVTVSS |

A-H.29

| | | |
|---|---|---|
| SEQ ID NO: | VH + VL | QVQLVQSGAEVKKPGSSVKVSCKASGHDFHLWYIHWVRQA |
| 159 | | PGQGLEWMGRISPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVAWHQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGDRVAWHQQKPG |
| 160 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFHLWYIHWVRQA |
| 161 | | PGQGLEWMGRISPGSGNVKYNEKFKGRVTITADTSTSTAYME |
| | | LSSLRSEDTAVYYCAGSYYSYDVLDYWGQGTTVTVSS |

A-H.31

| | | |
|---|---|---|
| SEQ ID NO: | VH + VL | QVQLVQSGAEVKKPGSSVKVSCKASGHDFKLTYIHWVRQAP |
| 162 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 163 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFKLTYIHWVRQAP |
| 164 | | GQGLEWMGRISAGSGNVKYNEKFKGRVTITADTSTSTAYME |
| | | LSSLRSEDTAVYYCAGSYYSYDVLDYWGQGTTVTVSS |

A-H.31

| | | |
|---|---|---|
| SEQ ID NO: | VH + VL | QVQLVQSGAEVKKPGSSVKVSCKASGTDFHLWYIHWVRQAP |
| 165 | | GQGLEWMGRVFAGSGSYRYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 166 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFHLWYIHWVRQAP |
| 167 | | GQGLEWMGRVFAGSGSYRYNEKFKGRVTITADTSTSTAYME |
| | | LSSLRSEDTAVYYCAGSYYSYDVLDYWGQGTTVTVSS |

A-H.32

| | | |
|---|---|---|
| SEQ ID NO: | VH + VL | QVQLVQSGAEVKKPGSSVKVSCKASGTDFDKIYIHWVRQAP |
| 168 | | GQGLEWMGRISAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVADRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVADRVAWYQQKPG |
| 169 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 170 | | GQGLEWMGRISAGSGNTKYNEKFKGRVTITADTSTSTAYME |
| | | LSSLRSEDTAVYYCAGSYYSYDVLDYWGQGTTVTVSS |

A-H.33

| | | |
|---|---|---|
| SEQ ID NO: | VH + VL | QVQLVQSGAEVKKPGSSVKVSCKASGTDFKLTYIHWVRQAP |
| 171 | | GQGLEWMGRISAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVEDRVAWYQQKPG |
| 172 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 173 | | GQGLEWMGRISAGSGNTKYNEKFKGRVTITADTSTSTAYME |
| | | LSSLRSEDTAVYYCAGSYYSYDVLDYWGQGTTVTVSS |

A-H.34

| | | |
|---|---|---|
| SEQ ID NO: | VH + VL | QVQLVQSGAEVKKPGSSVKVSCKASGTDFRLTYIHWVRQAP |
| 174 | | GQGLEWMGRISPGSGNTKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVGNRVAWYQQKPGKAPKALIYSSSHRYKGVPSRFSGSGSG |
| | | TEFTLTISSLQPEDFATYFCQQFKSYPLTFGQGTKLEIK |
| SEQ ID NO: | VL | DIQMTQSPSFLSASVGDRVTITCKASQNVGNRVAWYQQKPG |
| 175 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFRLTYIHWVRQAP |
| 176 | | GQGLEWMGRISPGSGNTKYNEKFKGRVTITADTSTSTAYMEL |
| | | SSLRSEDTAVYYCAGSYYSYDVLDYWGQGTTVTVSS |

A-H.35

| | | |
|---|---|---|
| SEQ ID NO: | VH + VL | QVQLVQSGAEVKKPGSSVKVSCKASGHDFDKTYIHWVRQAP |
| 177 | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVEDRVAWYQQKPG |
| 178 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKTYIHWVRQAP |
| 179 | | GQGLEWMGRVSAGSGNVKYNEKFKGRVTITADTSTSTAYME |
| | | LSSLRSEDTAVYYCAGSYYSYDVLDYWGQGTTVTVSS |

A-H.36

| | | |
|---|---|---|
| SEQ ID NO: | VH + VL | QVQLVQSGAEVKKPGSSVKVSCKASGHDFKLTYIHWVRQAP |
| 180 | | GQGLEWMGRVSPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVEDRVAWHQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVEDRVAWHQQKPG |
| 181 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFKLTYIHWVRQAP |
| 182 | | GQGLEWMGRVSPGSGNTKYNEKFKGRVTITADTSTSTAYME |
| | | LSSLRSEDTAVYYCAGSYYSYDVLDYWGQGTTVTVSS |

A-H.37

| | | |
|---|---|---|
| SEQ ID NO: | VH + VL | QVQLVQSGAEVKKPGSSVKVSCKASGHDFDKTYIHWVRQAP |
| 183 | | GQGLEWMGRIYPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVADRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVADRVAWYQQKPG |
| 184 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKTYIHWVRQAP |
| 185 | | GQGLEWMGRIYPGSGNVKYNEKFKGRVTITADTSTSTAYME |
| | | LSSLRSEDTAVYYCAGSYYSYDVLDYWGQGTTVTVSS |

A-H.38

| | | |
|---|---|---|
| SEQ ID NO: | VH + VL | QVQLVQSGAEVKKPGSSVKVSCKASGTDFDKTYIHWVRQAP |
| 186 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 187 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKTYIHWVRQAP |
| 188 | | GQGLEWMGRISAGSGNVKYNEKFKGRVTITADTSTSTAYME |
| | | LSSLRSEDTAVYYCAGSYYSYDVLDYWGQGTTVTVSS |

A-H.39

| | | |
|---|---|---|
| SEQ ID NO: | VH + VL | QVQLVQSGAEVKKPGSSVKVSCKASGTDFDKIYIHWVRQAP |
| 189 | | GQGLEWMGRISAGSGNIKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVDDRVAWYQQKPGKAPKALIYSSSHRYKGVPSRFSGSGSG |
| | | TEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 190 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 191 | | GQGLEWMGRISAGSGNIKYNEKFKGRVTITADTSTSTAYMEL |
| | | SSLRSEDTAVYYCAGSYYSYDVLDYWGQGTTVTVSS |

A-H.40

| | | |
|---|---|---|
| SEQ ID NO: | VH+VL | QVQLVQSGAEVKKPGSSVKVSCKASGTDFDKIYIHWVRQAP |
| 192 | | GQGLEWMGRISAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVGDRVAWYQQKPG |
| 193 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFDKIYIHWVRQAP |
| 194 | | GQGLEWMGRISAGSGNVKYNEKFKGRVTITADTSTSTAYME |
| | | LSSLRSEDTAVYYCAGSYYSYDVLDYWGQGTTVTVSS |

A-H.41

| | | |
|---|---|---|
| SEQ ID NO: | VH+VL | QVQLVQSGAEVKKPGSSVKVSCKASGGTFKLTYIHWVRQAP |
| 195 | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDDRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDDRVAWYQQKPG |
| 196 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGGTFKLTYIHWVRQAP |
| 197 | | GQGLEWMGRVSAGSGNVKYNEKFKGRVTITADTSTSTAYME |
| | | LSSLRSEDTAVYYCAGSYYSYDVLDYWGQGTTVTVSS |

A-H.42

| | | |
|---|---|---|
| SEQ ID NO: | VH+VL | QVQLVQSGAEVKKPGSSVKVSCKASGTDFKLTYIHWVRQAP |
| 198 | | GQGLEWMGRISPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNRVAWHQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDNRVAWHQQKPG |
| 199 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGTDFKLTYIHWVRQAP |
| 200 | | GQGLEWMGRISPGSGNVKYNEKFKGRVTITADTSTSTAYME |
| | | LSSLRSEDTAVYYCAGSYYSYDVLDYWGQGTTVTVSS |

A-H.43

| | | |
|---|---|---|
| SEQ ID NO: | VH+VL | QVQLVQSGAEVKKPGSSVKVSCKASGHDFDKFYIHWVRQAP |
| 348 | | GQGLEWMGRVSAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNRVAWYQQKPGKAPKALIYSSSHRYKGVPSRFSGSGS |
| | | GTEFTLTISSLQPEDFATYFCQQFKSYPLTFGQGTKLEIK |
| SEQ ID NO: | VL | DIQMTQSPSFLSASVGDRVTITCKASQNVDNRVAWYQQKPG |
| 349 | | KAPKALIYSSSHRYKGVPSRFSGSGSGTEFTLTISSLQPEDFAT |
| | | YFCQQFKSYPLTFGQGTKLEIK |
| SEQ ID NO: | VH | QVQLVQSGAEVKKPGSSVKVSCKASGHDFDKFYIHWVRQAP |
| 350 | | GQGLEWMGRVSAGSGNTKYNEKFKGRVTITADTSTSTAYME |
| | | LSSLRSEDTAVYYCAGSYYSYDVLDYWGQGTTVTVSS |

A-H.44

| | | |
|---|---|---|
| SEQ ID NO: | VH + VL | QVQLVQSGAEVKKPGSSVKVSCKASGTDFDKFYIHWVRQAP |
| 351 | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGDRVVWYQQKPGKAPKALIYSSSHRYKGVPSRFSGSGS |
| | | GTEFTLTISSLQPEDFATYFCQQFKSYPLTFGQGTKLEIK |
| SEQ ID NO: | VH | QVQLVQSGAEVKKPGSSVKVSCKASGTDFDKFYIHWVRQAP |
| 205 | | GQGLEWMGRVSAGSGNVKYNEKFKGRVTITADTSTSTAYME |
| | | LSSLRSEDTAVYYCAGSYYSYDVLDYWGQGTTVTVSS |

A-H.45

| | | |
|---|---|---|
| SEQ ID NO: | VH + VL | QVQLVQSGAEVKKPGSSVKVSCKASGYSFTTYYIHWVRQAP |
| 352 | | GQGLEWMGWFSAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 353 | | GQGLEWMGWFSAGSGNTKYNEKFKGRVTITADTSTSTAYME |
| | | LSSLRSEDTAVYYCAVSYYSYDVLDYWGQGTTVTVSS |

A-H.46

| | | |
|---|---|---|
| SEQ ID NO: | VH + VL | QVQLVQSGAEVKKPGSSVKVSCKASGYSFTTYYIHWVRQAP |
| 354 | | GQGLEWMGWFSAGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 355 | | GQGLEWMGWFSAGSGNTKYNEKFKGRVTITADTSTSTAYME |
| | | LSSLRSEDTAVYYCAGSYYSYDVLDYWGQGTTVTVSS |

A-H.47

| | | |
|---|---|---|
| SEQ ID NO: | VH + VL | QVQLVQSGAEVKKPGSSVKVSCKASGYSFTTYYIHWVRQAP |
| 356 | | GQGLEWMGWFFPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 357 | | GQGLEWMGWFFPGSGNTKYNEKFKGRVTITADTSTSTAYME |
| | | LSSLRSEDTAVYYCAGSYYSYDVLDYWGQGTTVTVSS |

A-H.48

| | | |
|---|---|---|
| SEQ ID NO: | VH + VL | QVQLVQSGAEVKKPGSSVKVSCKASGYSFTTYYIHWVRQAP |
| 358 | | GQGLEWMGWFSPGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 359 | | GQGLEWMGWFSPGSGNTKYNEKFKGRVTITADTSTSTAYME |
| | | LSSLRSEDTAVYYCAVSYYSYDVLDYWGQGTTVTVSS |

A-H.49

| | | |
|---|---|---|
| SEQ ID NO: | VH + VL | QVQLVQSGAEVKKPGSSVKVSCKASGYSFTTYYIHWVRQAP |
| 360 | | GQGLEWMGWFSPGSGNTKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 215 | | GQGLEWMGWFSPGSGNTKYNEKFKGRVTITADTSTSTAYME |
| | | LSSLRSEDTAVYYCAGSYYSYDVLDYWGQGTTVTVSS |

A-H.50

| | | |
|---|---|---|
| SEQ ID NO: | VH + VL | QVQLVQSGAEVKKPGSSVKVSCKASGYSFTTYYIHWVRQAP |
| 216 | | GQGLEWMGRIFPGSGNIKYNEKFKGRVTITADTSTSTAYMEL |
| | | SSLRSEDTAVYYCAGSYYSYDVLDYWGQGTTVTVSSGGGGS |
| | | GGGGSGGGGSGGGGSDIQMTQSPSFLSASVGDRVTITCKASQ |
| | | NVGINVVWHQQKPGKAPKALIYSSSHRYSGVPSRFSGSGSGT |
| | | EFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 361 | | GQGLEWMGRIFPGSGNIKYNEKFKGRVTITADTSTSTAYMEL |
| | | SSLRSEDTAVYYCAGSYYSYDVLDYWGQGTTVTVSS |

A-H.51

| | | |
|---|---|---|
| SEQ ID NO: | VH + VL | QVQLVQSGAEVKKPGSSVKVSCKASGYSFTTYYIHWVRQAP |
| 362 | | GQGLEWMGWFFPGSGNIKYNEKFKGRVTITADTSTSTAYME |
| | | LSSLRSEDTAVYYCAGSIYSAGVLDYWGQGTTVTVSSGGGGS |
| | | GGGGSGGGGSGGGGSDIQMTQSPSFLSASVGDRVTITCKASQ |
| | | NVGINVVWHQQKPGKAPKALIYSSSHRYSGVPSRFSGSGSGT |
| | | EFTLTISSLQPEDFATYFCQQFKSYPLTFGQGTKLEIK |
| SEQ ID NO: | VH | QVQLVQSGAEVKKPGSSVKVSCKASGYSFTTYYIHWVRQAP |
| 219 | | GQGLEWMGWFFPGSGNIKYNEKFKGRVTITADTSTSTAYME |
| | | LSSLRSEDTAVYYCAGSIYSAGVLDYWGQGTTVTVSS |

A-H.52

| | | |
|---|---|---|
| SEQ ID NO: | VH + VL | QVQLVQSGAEVKKPGSSVKVSCKASGYSFTLGYIHWVRQAP |
| 220 | | GQGLEWMGWFFPGSGNIKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFTLGYIHWVRQAP |
| 363 | | GQGLEWMGWFFPGSGNIKYNEKFKGRVTITADTSTSTAYME |
| | | LSSLRSEDTAVYYCAGSYYSYDVLDYWGQGTTVTVSS |

A-H.53

| | | |
|---|---|---|
| SEQ ID NO: | VH + VL | QVQLVQSGAEVKKPGSSVKVSCKASGYSFRLTYIHWVRQAP |
| 222 | | GQGLEWMGWFFPGSGNIKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGINVVWHQQKPGKAPKALIYSSSHRYSGVPSRFSGSGSG |
| | | TEFTLTISSLQPEDFATYFCQQFKSYPLTFGQGTKLEIK |
| SEQ ID NO: | VH | QVQLVQSGAEVKKPGSSVKVSCKASGYSFRLTYIHWVRQAP |
| 364 | | GQGLEWMGWFFPGSGNIKYNEKFKGRVTITADTSTSTAYME |
| | | LSSLRSEDTAVYYCAGSYYSYDVLDYWGQGTTVTVSS |

A-H.54

| | | |
|---|---|---|
| SEQ ID NO: | VH + VL | QVQLVQSGAEVKKPGSSVKVSCKASGYSFHNWYIHWVRQA |
| 365 | | PGQGLEWMGWFFPGSGNIKYNEKFKGRVTITADTSTSTAYM |
| | | ELSSLRSEDTAVYYCAGSYYSYDVLDYWGQGTTVTVSSGGG |
| | | GSGGGGSGGGGSGGGGSDIQMTQSPSFLSASVGDRVTITCKA |
| | | SQNVGINVVWHQQKPGKAPKALIYSSSHRYSGVPSRFSGSGS |
| | | GTEFTLTISSLQPEDFATYFCQQFKSYPLTFGQGTKLEIK |
| SEQ ID NO: | VH | QVQLVQSGAEVKKPGSSVKVSCKASGYSFHNWYIHWVRQA |
| 366 | | PGQGLEWMGWFFPGSGNIKYNEKFKGRVTITADTSTSTAYM |
| | | ELSSLRSEDTAVYYCAGSYYSYDVLDYWGQGTTVTVSS |

A-H.55 antibody

| | | |
|---|---|---|
| SEQ ID NO: 3 | HC CDR1 (Combined) | GYSFTTYYIH |
| SEQ ID NO: 4 | HC CDR2 (Combined) | WFFPGSGNIK YNEKFKG |
| SEQ ID NO: 5 | HC CDR3 (Combined) | SYYSYDVLDY |
| SEQ ID NO: 45 | HC CDR1 (Kabat) | TYYIH |
| SEQ ID NO: 46 | HC CDR2 (Kabat) | WFFPGSGNIK YNEKFKG |
| SEQ ID NO: 47 | HC CDR3 (Kabat) | SYYSYDVLDY |
| SEQ ID NO: 48 | HC CDR1 (Chothia) | GYSFTTY |
| SEQ ID NO: 49 | HC CDR2 (Chothia) | FPGSGN |
| SEQ ID NO: 50 | HC CDR3 (Chothia) | SYYSYDVLDY |
| SEQ ID NO: | VH | QVQLVQSGAEVKKPGSSVKVSCKASGYSFTTYYIHWVRQAP |
| 1100 | | GQGLEWMGWFFPGSGNIKYNEKFKGRVTITADTSTSTAYME |
| | | LSSLRSEDTAVYYCAGSYYSYDVLDYWGQGTTVTVSS |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 51 | LC CDR1 (Kabat) | KASQNVGINVV |
| SEQ ID NO: 52 | LC CDR2 (Kabat) | SSSHRYS |
| SEQ ID NO: 53 | LC CDR3 (Kabat) | QQFKSYPLT |
| SEQ ID NO: 54 | LC CDR1 (Chothia) | KASQNVGINVV |
| SEQ ID NO: 55 | LC CDR2 (chothia) | SSSHRYS |
| SEQ ID NO: 56 | LC CDR3 (chothia) | QQFKSYPLT |
| SEQ ID NO: | VL | QSVLTQPPSVSEAPRQRVTISCKASQNVGINVVWHQQLPGKA |
| 1101 | | PKALIYSSSHRYSGVSDRFSGSGSGTSFSLAISGLQSEDEADYF |
| | | CQQFKSYPLTFGTGTKVTVL |

A-H.56

| | | |
|---|---|---|
| SEQ ID NO: | VH + VL (ScFv) | QVQLVQSGAEVKKPGSSVKVSCKASGHDFDKFYIHWVRQAP |
| 1309 | | GQGLEWMGRVSAGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVGNRVAWYQQKPGKAPKALIYSSSHRYKGVPSRFSGSGS |
| | | GTEFTLTISSLQPEDFATYFCQQFKSYPLTFGQGTKLEIK |

A-H.57

| | | |
|---|---|---|
| SEQ ID NO: | VH + VL (ScFv) | QVQLVQSGAEVKKPGSSVKVSCKASGHDFRLTYIHWVRQAP |
| 1326 | | GQGLEWMGRVSAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGDRVVWHQQKPGKAPKALIYSSSHRYKGVPSRFSGSGS |
| | | GTEFTLTISSLQPEDFATYFCQQFKSYPLTFGQGTKLEIK |

A-H.58

| | | |
|---|---|---|
| SEQ ID NO: | VH + VL (ScFv) | QVQLVQSGAEVKKPGSSVKVSCKASGHDFRLTYIHWVRQAP |
| 1327 | | GQGLEWMGRVSAGSGNVKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGNRVVWHQQKPGKAPKALIYSSSHRYKGVPSRFSGSGS |
| | | GTEFTLTISSLQPEDFATYFCQQFKSYPLTFGQGTKLEIK |

A-H.59

| | | |
|---|---|---|
| SEQ ID NO: | VH + VL (ScFv) | QVQLVQSGAEVKKPGSSVKVSCKASGHDFRLTYIHWVRQAP |
| 1328 | | GQGLEWMGRIYAGSGNVKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVADRVVWHQQKPGKAPKALIYSSSHRYKGVPSRFSGSGS |
| | | GTEFTLTISSLQPEDFATYFCQQFKSYPLTFGQGTKLEIK |

A-H.60

| | | |
|---|---|---|
| SEQ ID NO: | VH + VL (ScFv) | QVQLVQSGAEVKKPGSSVKVSCKASGHDFKLTYIHWVRQAP |
| 1329 | | GQGLEWMGRVSAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGDRVAWHQQKPGKAPKALIYSSSHRYKGVPSRFSGSGS |
| | | GTEFTLTISSLQPEDFATYFCQQFKSYPLTFGQGTKLEIK |

A-H.61

| | | |
|---|---|---|
| SEQ ID NO: | VH + VL (ScFv) | QVQLVQSGAEVKKPGSSVKVSCKASGHDFKLTYIHWVRQAP |
| 1330 | | GQGLEWMGRVSAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVDNRVAWHQQKPGKAPKALIYSSSHRYKGVPSRFSGSGS |
| | | GTEFTLTISSLQPEDFATYFCQQFKSYPLTFGQGTKLEIK |

A-H.62

| | | |
|---|---|---|
| SEQ ID NO: | VH + VL (ScFv) | QVQLVQSGAEVKKPGSSVKVSCKASGHDFRLTYIHWVRQAP |
| 1331 | | GQGLEWMGRVSAGSGNVKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVADRVVWHQQKPGKAPKALIYSSSHRYKGVPSRFSGSGS |
| | | GTEFTLTISSLQPEDFATYFCQQFKSYPLTFGQGTKLEIK |

A-H.63

| | | |
|---|---|---|
| SEQ ID NO: | VH + VL (ScFv) | QVQLVQSGAEVKKPGSSVKVSCKASGHDFRLTYIHWVRQAP |
| 1332 | | GQGLEWMGRVYAGSGNTKYNEKFKGRVTITADTSTSTAYM |
| | | ELSSLRSEDTAVYYCAVSYYSYDVLDYWGQGTTVTVSSGGG |
| | | GSGGGGSGGGGSGGGGSDIQMTQSPSFLSASVGDRVTITCKA |
| | | SQNVEDRVVWHQQKPGKAPKALIYSSSHRYKGVPSRFSGSGS |
| | | GTEFTLTISSLQPEDFATYFCQQFKSYPLTFGQGTKLEIK |

A-H.64

| | | |
|---|---|---|
| SEQ ID NO: | VH + VL (ScFv) | QVQLVQSGAEVKKPGSSVKVSCKASGHDFKLTYIHWVRQAP |
| 1333 | | GQGLEWMGRVSAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVADRVVWHQQKPGKAPKALIYSSSHRYKGVPSRFSGSGS |
| | | GTEFTLTISSLQPEDFATYFCQQFKSYPLTFGQGTKLEIK |

A-H.65

| | | |
|---|---|---|
| SEQ ID NO: | VH + VL (ScFv) | QVQLVQSGAEVKKPGSSVKVSCKASGHDFKLTYIHWVRQAP |
| 1334 | | GQGLEWMGRISAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGDRVVWHQQKPGKAPKALIYSSSHRYKGVPSRFSGSGS |
| | | GTEFTLTISSLQPEDFATYFCQQFKSYPLTFGQGTKLEIK |

A-H.66

| | | |
|---|---|---|
| SEQ ID NO: | VH + VL (ScFv) | QVQLVQSGAEVKKPGSSVKVSCKASGHDFKLTYIHWVRQAP |
| 1335 | | GQGLEWMGRIYAGSGNTKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVGDRVVWHQQKPGKAPKALIYSSSHRYKGVPSRFSGSGS |
| | | GTEFTLTISSLQPEDFATYFCQQFKSYPLTFGQGTKLEIK |

A-H.67

| | | |
|---|---|---|
| SEQ ID NO: | VH + VL (ScFv) | QVQLVQSGAEVKKPGSSVKVSCKASGTDFKLTYIHWVRQAP |
| 1336 | | GQGLEWMGRIFPGSGNVKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVDNRVAWYQQKPGKAPKALIYSSSHRYKGVPSRFSGSGS |
| | | GTEFTLTISSLQPEDFATYFCQQFKSYPLTFGQGTKLEIK |

A-H.68

| | | |
|---|---|---|
| SEQ ID NO: | VH+ VL (ScFv) | QVQLVQSGAEVKKPGSSVKVSCKASGHDFRLTYIHWVRQAP |
| 1337 | | GQGLEWMGRISAGSGNVKYNEKFKGRVTITADTSTSTAYME |
| | | LSSLRSEDTAVYYCAVSYYSYDVLDYWGQGTTVTVSSGGGG |
| | | SGGGGSGGGGSGGGGSDIQMTQSPSFLSASVGDRVTITCKAS |
| | | QNVADRVAWYQQKPGKAPKALIYSSSHRYKGVPSRFSGSGS |
| | | GTEFTLTISSLQPEDFATYFCQQFKSYPLTFGQGTKLEIK |

A-H.69 (also referred to as A-H.13)

| | | |
|---|---|---|
| SEQ ID NO: | VH + VL (ScFv) | QVQLVQSGAEVKKPGSSVKVSCKASGTDFKLTYIHWVRQAP |
| 110 | | GQGLEWMGRIFPGSGNVKYNEKFKGRVTITADTSTSTAYME |
| | | LSSLRSEDTAVYYCAGSYYSYDVLDYWGQGTTVTVSSGGGG |
| | | SGGGGSGGGGSGGGGSDIQMTQSPSFLSASVGDRVTITCKAS |
| | | QNVDNRVAWYQQKPGKAPKALIYSSSHRYKGVPSRFSGSGS |
| | | GTEFTLTISSLQPEDFATYFCQQFKSYPLTFGQGTKLEIK |

A-H humanized-matured VH

| | | |
|---|---|---|
| SEQ ID NO: | VH-humanized matured 1 | QVQLVQSGAEVKKPGSSVKVSCKASGTDFKLTYIHWVRQAP |
| 1310 | | GQGLEWMGRIFPGSGNVKYNEKFKGRVTITADTSTSTAYME |
| | | LSSLRSEDTAVYYCAGSYYSYDVLDYWGQGTTVTVSS |
| SEQ ID NO: | VH-humanized matured 2 | QVQLVQSGAEVKKPGSSVKVSCKASGTDFKLTYIHWVRQAP |
| 1311 | | GQGLEWMGRIFPGSGNVKYNEKFKGRVTITADTSTSTAYME |
| | | LSSLRSEDTAVYYCAVSYYSYDVLDYWGQGTTVTVSS |
| SEQ ID NO: | VH-humanized matured 3 | QVQLVQSGAEVKKPGSSVKVSCKASGHDFRLTYIHWVRQAP |
| 1312 | | GQGLEWMGRISAGSGNVKYNEKFKGRVTITADTSTSTAYME |
| | | LSSLRSEDTAVYYCAVSYYSYDVLDYWGQGTTVTVSS |

A-H humanized-matured VL

| | | |
|---|---|---|
| SEQ ID NO: | VL-humanized matured 1 | DIQMTQSPSFLSASVGDRVTITCKASQNVDNRVAWYQQKPG |
| 1313 | | KAPKALIYSSSHRYKGVPSRFSGSGSGTEFTLTISSLQPEDFAT |
| | | YFCQQFKSYPLTFGQGTKLEIK |
| SEQ ID NO: | VL-humanized matured 2 | DIQMTQSPSFLSASVGDRVTITCKASQNVADRVAWYQQKPG |
| 1314 | | KAPKALIYSSSHRYKGVPSRFSGSGSGTEFTLTISSLQPEDFAT |
| | | YFCQQFKSYPLTFGQGTKLEIK |

A-H.70

| | | |
|---|---|---|
| SEQ ID NO: | HC CDR1 | GHDFRLTYIH |
| 3650 | | |
| SEQ ID NO: | HC CDR2 | RVSAGSGNVKYNEKFKG |
| 3651 | | |
| SEQ ID NO: 5 | HC CDR3 | SYYSYDVLDY |
| SEQ ID NO: | LC CDR1 | KASQNVGNRVV |
| 3652 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWVRQAP |
| 1346 | (CDRs underlined) | GQGLEWMGRVSAGSGNVKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVGNRVVWHQQKPG |
| 1347 | (CDRs underlined) | KAPKALIYSSSHRYKGVPSRFSGSGSGTEFTLTISSLQPEDFAT |
| | | YFCQQFKSYPLTFGQGTKLEIK |

A-H.71

| | | |
|---|---|---|
| SEQ ID NO: | HC CDR1 | GHDFRLTYIH |
| 3650 | | |
| SEQ ID NO: | HC CDR2 | RIYAGSGNVKYNEKFKG |
| 3654 | | |
| SEQ ID NO: 5 | HC CDR3 | SYYSYDVLDY |
| SEQ ID NO: | LC CDR1 | KASQNVADRVV |
| 3655 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWVRQAP |
| 1348 | (CDRs underlined) | GQGLEWMGRIYAGSGNVKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVADRVVWHQQKPG |
| 1349 | (CDRs underlined) | KAPKALIYSSSHRYKGVPSRFSGSGSGTEFTLTISSLQPEDFAT |
| | | YFCQQFKSYPLTFGQGTKLEIK |

A-H.72

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVGDRVA |
| 3658 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1350 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVGDRVAWHQQKPG |
| 1351 | (CDRs underlined) | KAPKALIYSSSHRYKGVPSRFSGSGSGTEFTLTISSLQPEDFAT |
| | | YFCQQFKSYPLTFGQGTKLEIK |

A-H.73

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1350 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1353 | (CDRs underlined) | KAPKALIYSSSHRYKGVPSRFSGSGSGTEFTLTISSLQPEDFAT |
| | | YFCQQFKSYPLTFGQGTKLEIK |

A-H.74

| | | |
|---|---|---|
| SEQ ID NO: | HC CDR1 | GHDFRLTYIH |
| 3650 | | |
| SEQ ID NO: | HC CDR2 | RVSAGSGNVKYNEKFKG |
| 3651 | | |
| SEQ ID NO: 5 | HC CDR3 | SYYSYDVLDY |
| SEQ ID NO: | LC CDR1 | KASQNVADRVV |
| 3655 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWVRQAP |
| 1346 | (CDRs underlined) | GQGLEWMGRVSAGSGNVKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVADRVVWHQQKPG |
| 1349 | (CDRs underlined) | KAPKALIYSSSHRYKGVPSRFSGSGSGTEFTLTISSLQPEDFAT |
| | | YFCQQFKSYPLTFGQGTKLEIK |

A-H.75

| | | |
|---|---|---|
| SEQ ID NO: | HC CDR1 | GHDFRLTYIH |
| 3650 | | |
| SEQ ID NO: | HC CDR2 | RVYAGSGNTKYNEKFKG |
| 3659 | | |
| SEQ ID NO: 5 | HC CDR3 | SYYSYDVLDY |
| SEQ ID NO: | LC CDR1 | KASQNVEDRVV |
| 3660 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFRLTYIHWVRQAP |
| 1356 | (CDRs underlined) | GQGLEWMGRVYAGSGNTKYNEKFKGRVTITADTSTSTAYM |
| | | ELSSLRSEDTAVYYCAVSYYSYDVLDYWGQGTTVTVSS |
| SEQ ID NO: | VL | DIQMTQSPSFLSASVGDRVTITCKASQNVEDRVVWHQQKPG |
| 1357 | (CDRs underlined) | KAPKALIYSSSHRYKGVPSRFSGSGSGTEFTLTISSLQPEDFAT |
| | | YFCQQFKSYPLTFGQGTKLEIK |

A-H.76

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVADRVV |
| 3655 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1350 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVADRVVWHQQKPG |
| 1349 | (CDRs underlined) | KAPKALIYSSSHRYKGVPSRFSGSGSGTEFTLTISSLQPEDFAT |
| | | YFCQQFKSYPLTFGQGTKLEIK |

A-H.77

| | | |
|---|---|---|
| SEQ ID NO: | HC CDR1 | GHDFKLTYIH |
| 3656 | | |
| SEQ ID NO: | HC CDR2 | RISAGSGNTKYNEKFKG |
| 3661 | | |
| SEQ ID NO: 5 | HC CDR3 | SYYSYDVLDY |
| SEQ ID NO: | LC CDR1 | KASQNVGDRVV |
| 3662 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1360 | (CDRs underlined) | GQGLEWMGRISAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVGDRVVWHQQKPG |
| 1361 | (CDRs underlined) | KAPKALIYSSSHRYKGVPSRFSGSGSGTEFTLTISSLQPEDFAT |
| | | YFCQQFKSYPLTFGQGTKLEIK |

A-H.78

| | | |
|---|---|---|
| SEQ ID NO: | HC CDR1 | GHDFKLTYIH |
| 3656 | | |
| SEQ ID NO: | HC CDR2 | RIYAGSGNTK YNEKFKG |
| 3663 | | |
| SEQ ID NO: 5 | HC CDR3 | SYYSYDVLDY |
| SEQ ID NO: | LC CDR1 | KASQNVGDRVV |
| 3662 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1362 | (CDRs underlined) | GQGLEWMGRIYAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVGDRVVWHQQKPG |
| 1361 | (CDRs underlined) | KAPKALIYSSSHRYKGVPSRFSGSGSGTEFTLTISSLQPEDFAT |
| | | YFCQQFKSYPLTFGQGTKLEIK |

A-H.79

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | RASQNVDNRLG |
| 3666 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1350 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCRASQNVDNRLGWHQQKPG |
| 1365 | (CDRs underlined) | KAPKALIYSSSHRYKGVPSRFSGSGSGTEFTLTISSLQPEDFAT |
| | | YFCQQFKSYPLTFGQGTKLEIK |

A-H.80

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | AASSLQK |
| 3668 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1350 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1367 | (CDRs underlined) | KAPKALIYAASSLQKGVPSRFSGSGSGTEFTLTISSLQPEDFAT |
| | | YFCQQFKSYPLTFGQGTKLEIK |

A-H.81

| | | |
|---|---|---|
| SEQ ID NO: | HC CDR1 | GHDFKLTYIH |
| 3656 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | SSSHRYK |
| 3653 | | |
| SEQ ID NO: | LC CDR3 | LQHNSYPLT |
| 3664 | | |
| SEQ ID NO: | VH | QVQLVQSGAEVKKPGSSVKVSCKASGHDFKLTYIHWVRQAP |
| 1350 | (CDRs underlined) | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1369 | (CDRs underlined) | KAPKALIYSSSHRYKGVPSRFSGSGSGTEFTLTISSLQPEDFAT |
| | | YFCLQHNSYPLTFGQGTKLEIK |

A-H.82

| | | |
|---|---|---|
| SEQ ID NO: | HC CDR1 | GHDFKLTYIH |
| 3656 | | |
| SEQ ID NO: | HC CDR2 | RVSAGSGNVNYAQKFQG |
| 3665 | | |
| SEQ ID NO: 5 | HC CDR3 | SYYSYDVLDY |
| SEQ ID NO: | LC CDR1 | RASQNVDNRLG |
| 3666 | | |
| SEQ ID NO: | LC CDR2 | SSSHRYK |
| 3653 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1370 | (CDRs underlined) | GQGLEWMGRVSAGSGNVNYAQKFQGRVTITADTSTSTAYM |
| | | ELSSLRSEDTAVYYCAVSYYSYDVLDYWGQGTTVTVSS |
| SEQ ID NO: | VL | DIQMTQSPSFLSASVGDRVTITCRASQNVDNRLGWHQQKPG |
| 1365 | (CDRs underlined) | KAPKALIYSSSHRYKGVPSRFSGSGSGTEFTLTISSLQPEDFAT |
| | | YFCQQFKSYPLTFGQGTKLEIK |

A-H.83

| | | |
|---|---|---|
| SEQ ID NO: | HC CDR1 | GHDFKLTYIH |
| 3656 | | |
| SEQ ID NO: | HC CDR2 | RVSAGSGNVNYAQKFQG |
| 3665 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | AASSLQK |
| 3668 | | |
| SEQ ID NO: 8 | LC CDR3 | QQFKSYPLT |
| SEQ ID NO: | VH | QVQLVQSGAEVKKPGSSVKVSCKASGHDFKLTYIHWVRQAP |
| 1370 | (CDRs underlined) | GQGLEWMGRVSAGSGNVNYAQKFQGRVTITADTSTSTAYM |
| | | ELSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1367 | (CDRs underlined) | KAPKALIYAASSLQKGVPSRFSGSGSGTEFTLTISSLQPEDFAT |
| | | YFCQQFKSYPLTFGQGTKLEIK |

A-H.84

| | | |
|---|---|---|
| SEQ ID NO: | HC CDR1 | GHDFKLTYIH |
| 3656 | | |
| SEQ ID NO: | HC CDR2 | RVSAGSGNVNYAQKFQG |
| 3665 | | |
| SEQ ID NO: 5 | HC CDR3 | SYYSYDVLDY |
| SEQ ID NO: | LC CDR1 | KASQNVDNRVA |
| 3667 | | |
| SEQ ID NO: | LC CDR2 | SSSHRYK |
| 3653 | | |
| SEQ ID NO: | LC CDR3 | LQHNSYPLT |
| 3664 | | |
| SEQ ID NO: | VH | QVQLVQSGAEVKKPGSSVKVSCKASGHDFKLTYIHWVRQAP |
| 1370 | (CDRs underlined) | GQGLEWMGRVSAGSGNVNYAQKFQGRVTITADTSTSTAYM |
| | | ELSSLRSEDTAVYYCAVSYYSYDVLDYWGQGTTVTVSS |
| SEQ ID NO: | VL | DIQMTQSPSFLSASVGDRVTITCKASQNVDNRVAWHQQKPG |
| 1369 | (CDRs underlined) | KAPKALIYSSSHRYKGVPSRFSGSGSGTEFTLTISSLQPEDFAT |
| | | YFCLQHNSYPLTFGQGTKLEIK |

A-H.85

| | | |
|---|---|---|
| SEQ ID NO: | HC CDR1 | GHDFRLTYIH |
| 3650 | | |
| SEQ ID NO: | HC CDR2 | RVSAGSGNTKYNEKFKG |
| 3657 | | |
| SEQ ID NO: 5 | HC CDR3 | SYYSYDVLDY |
| SEQ ID NO: | LC CDR1 | KASQNVGDRVV |
| 3662 | | |
| SEQ ID NO: | LC CDR2 | SSSHRYK |
| 3653 | | |
| SEQ ID NO: 8 | LC CDR3 | QQFKSYPLT |
| SEQ ID NO: | VH (CDRs underlined) | QVQLVQSGAEVKKPGSSVKVSCKASGHDFRLTYIHWVRQAP |
| 1344 | | GQGLEWMGRVSAGSGNTKYNEKFKGRVTITADTSTSTAYME |
| | | LSSLRSEDTAVYYCAVSYYSYDVLDYWGQGTTVTVSS |
| SEQ ID NO: | VL | DIQMTQSPSFLSASVGDRVTITCKASQNVGDRVVWHQQKPG |
| 1361 | (CDRs underlined) | KAPKALIYSSSHRYKGVPSRFSGSGSGTEFTLTISSLQPEDFAT |
| | | YFCQQFKSYPLTFGQGTKLEIK |

TABLE 32
Amino acid and nucleotide sequences for murine and humanized antibody molecules
which bind to TCRVB 12, e.g., TCRVB 12-3 or TCRVB 12-4. The antibody molecules include
murine mAb Antibody B and humanized mAb Antibody B-H.1 to B-H.6. The amino acid the heavy
and light chain CDRs, and the amino acid and nucleotide sequences of the heavy and
light chain variable regions, and the heavy and light chains are shown.
also referred to as 16G8
Antibody B (murine), also referred to as 16G8

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 57 | HC CDR1 (Kabat) | NFGMH |
| SEQ ID NO: 58 | HC CDR2 (Kabat) | YISSGSSTIYYADTLKG |
| SEQ ID NO: 59 | HC CDR3 (Kabat) | RGEGAMDY |
| SEQ ID NO: 60 | HC CDR1 (Chothia) | GFTFSNF |
| SEQ ID NO: 61 | HC CDR2 (Chothia) | SSGSST |
| SEQ ID NO: 62 | HC CDR3 (Chothia) | RGEGAMDY |
| SEQ ID NO: 15 | VH | DVQLVESGGGLVQPGGSRKLSCAASGFTFSNFGMHWVRQA |
| | | PDKGLEWVAYISSGSSTIYYADTLKGRFTISRDNPKNTLFL |
| | | QMTSLRSEDTAMYYCARRGEGAMDYWGQGTSVTVSS |
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3(Combined) | QQFTSSPFT |
| SEQ ID NO: 63 | LC CDR1 (Kabat) | RASSSVNYIY |
| SEQ ID NO: 64 | LC CDR2 (Kabat) | YTSNLAP |
| SEQ ID NO: 65 | LC CDR3 (Kabat) | QQFTSSPFT |
| SEQ ID NO: 66 | LC CDR1 (Chothia) | RASSSVNYIY |
| SEQ ID NO: 67 | LC CDR2 (Chothia) | YTSNLAP |
| SEQ ID NO: 68 | LC CDR3 (Chothia) | QQFTSSPFT |
| SEQ ID NO: 16 | VL | ENVLTQSPAIMSASLGEKVTMSCRASSSVNYIYWYQQKSD |
| | | ASPKLWIYYTSNLAPGVPTRFSGSGSGNSYSLTISSMEGE |
| | | DAATYYCQQFTSSPFTFGSGTKLEIK |

Antibody B humanized (B-H)
Antibody B-H.1A HC-1

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: | VH | EVQLVESGGGLVQPGGSLRLSCAASGFTFSNFGMHWVRQA |
| 3438 | | PGKGLEWVSYISSGSSTIYYADTLKGRFTISRDNAKNSLY |
| | | LQMNSLRAEDTAVYYCARRGEGAMDYWGQGTTVTVSS |
| SEQ ID NO: 31 | DNA VH | GAGGTGCAGCTGGTTGAATCTGGCGGAGGATTGGTTCAG |
| | | CCTGGCGGCTCTCTGAGACTGTCTTGTGCCGCTTCTGGCT |
| | | TCACCTTCTCCAACTTCGGCATGCACTGGGTCCGACAGG |
| | | CCCCTGGAAAAGGACTGGAATGGGTGTCCTACATCTCCT |
| | | CCGGCTCCTCCACCATCTACTACGCTGACACCCTGAAGG |
| | | GCAGATTCACCATCTCTCGGGACAACGCCAAGAACTCCC |
| | | TGTACCTGCAGATGAACAGCCTGAGAGCCGAGGACACC |
| | | GCCGTGTACTACTGTGCTAGAAGAGGCGAGGGCGCCATG |
| | | GATTATTGGGGCCAGGGAACCACAGTGACCGTGTCTAGC |

Antibody B-H.1B HC-2

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 25 | VH | EVQLVESGGGLVQPGGSLRLSCAASGFTFSNFGMHWVRQA |
| | | PGKGLEWVSYISSGSSTIYYADTLKGRFTISRDNSKNTLY |
| | | LQMNSLRAEDTAVYYCARRGEGAMDYWGQGTTVTVSS |
| SEQ ID NO: 32 | DNA VH | GAGGTGCAGCTGGTTGAATCTGGCGGAGGATTGGTTCAG |
| | | CCTGGCGGCTCTCTGAGACTGTCTTGTGCCGCTTCTGGCT |
| | | TCACCTTCTCCAACTTCGGCATGCACTGGGTCCGACAGG |
| | | CCCCTGGAAAAGGACTGGAATGGGTGTCCTACATCTCCT |
| | | CCGGCTCCTCCACCATCTACTACGCTGACACCCTGAAGG |
| | | GCAGATTCACCATCAGCCGGGACAACTCCAAGAACACCC |
| | | TGTACCTGCAGATGAACTCCCTGAGAGCCGAGGACACCG |
| | | CCGTGTACTACTGTGCTAGAAGAGGCGAGGGCGCCATGG |
| | | ATTATTGGGGCCAGGGAACCACAGTGACCGTGTCTAGC |

Antibody B-H.1C HC-3

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 23 | VH | QVQLVESGGGVVQPGRSLRLSCAASGFTFSNFGMHWVRQ |
| | | APGKGLEWVAYISSGSSTIYYADTLKGRFTISRDNSKNT |
| | | LYLQMNSLRAEDTAVYYCARRGEGAMDYWGQGTTVTVSS |
| SEQ ID NO: 33 | DNA VH | CAGGTGCAGCTGGTGGAATCTGGTGGCGGAGTTGTGCAG |
| | | CCTGGCAGATCCCTGAGACTGTCTTGTGCCGCCTCTGGCT |
| | | TCACCTTCTCCAACTTCGGCATGCACTGGGTCCGACAGG |
| | | CCCCTGGAAAAGGATTGGAGTGGGTCGCCTACATCTCCT |
| | | CCGGCTCCTCCACCATCTACTACGCTGACACCCTGAAGG |
| | | GCAGATTCACCATCAGCCGGGACAACTCCAAGAACACCC |
| | | TGTACCTGCAGATGAACTCCCTGAGAGCCGAGGACACCG |
| | | CCGTGTACTACTGTGCTAGAAGAGGCGAGGGCGCCATGG |
| | | ATTATTGGGGCCAGGGAACCACAGTGACCGTGTCTAGC |

Antibody B-H.1D LC-1

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3(Combined) | QQFTSSPFT |
| SEQ ID NO: | VL | DNQLTQSPSFLSASVGDRVTITCRASSSVNYIYWYQQKPGK |
| 8669 | | APKLLIYYTSNLAPGVPSRFSGSGSGNEYTLTISSLQPEDF |
| | | ATYYCQQFTSSPFTFGQGTKLEIK |
| SEQ ID NO: | DNA VL | GATAACCAGCTGACCCAGTCTCCTAGCTTCCTGTCTGCCT |
| 8674 | | CTGTGGGCGACAGAGTGACAATTACCTGCCGGGCCTCCT |
| | | CCTCCGTGAACTACATCTACTGGTATCAGCAGAAGCCCG |
| | | GCAAGGCCCCTAAGCTGCTGATCTACTACACCTCCAATC |
| | | TGGCCCCTGGCGTGCCCTCTAGATTTTCCGGATCTGGCTC |
| | | CGGCAACGAGTATACCCTGACAATCTCCAGCCTGCAGCC |
| | | TGAGGACTTCGCCACCTACTACTGCCAGCAGTTCACCTC |
| | | CTCTCCATTCACCTTTGGCCAGGGCACCAAGCTGGAAAT |
| | | CAAA |

Antibody B-H.1E LC-2

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3(Combined) | QQFTSSPFT |
| SEQ ID NO: 27 | VL | DNQLTQSPSSLSASVGDRVTITCRASSSVNYIYWYQQKPGK |
| | | APKLLIYYTSNLAPGVPSRFSGSGSGNDYTLTISSLQPEDF |
| | | ATYYCQQFTSSPFTFGQGTKLEIK |
| SEQ ID NO: 35 | DNA VL | ATAACCAGCTGACCCAGTCTCCTTCCAGCCTGTCTGCTTC |
| | | TGTGGGCGACAGAGTGACAATTACCTGCCGGGCCTCCTC |
| | | CTCCGTGAACTACATCTACTGGTATCAGCAGAAGCCCGG |
| | | CAAGGCCCCTAAGCTGCTGATCTACTACACCTCCAATCT |
| | | GGCCCCTGGCGTGCCCTCTAGATTTTCCGGATCTGGCTCC |
| | | GGCAACGACTATACCCTGACAATCTCCAGCCTGCAGCCT |
| | | GAGGACTTCGCCACCTACTACTGCCAGCAGTTCACCTCC |
| | | TCTCCATTCACCTTTGGCCAGGGCACCAAGCTGGAAATC |
| | | AAA |

Antibody B-H.1F LC-3

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3(Combined) | QQFTSSPFT |
| SEQ ID NO: | VL | ENVLTQSPATLSVSPGERATLSCRASSSVNYIYWYQQKPGQ |
| 8671 | | APRLLIYYTSNLAPGIPARFSGSGSGNEYTLTISSLQSEDF |
| | | AVYYCQQFTSSPFTFGQGTKLEIK |
| SEQ ID NO: | DNA VL | GAGAATGTGCTGACCCAGTCTCCTGCCACACTGTCTGTT |
| 8675 | | AGCCCTGGCGAGAGAGCTACCCTGAGCTGCAGAGCCTCT |
| | | TCCTCCGTGAACTACATCTACTGGTATCAGCAGAAGCCC |
| | | GGCCAGGCTCCTAGACTGCTGATCTACTACACCTCCAAT |
| | | CTGGCCCCTGGCATCCCTGCCAGATTTTCCGGATCTGGCT |
| | | CCGGCAACGAGTATACCCTGACCATCTCCAGCCTGCAGT |
| | | CCGAGGACTTTGCTGTGTACTATTGCCAGCAGTTCACAA |
| | | GCAGCCCTTTCACCTTTGGCCAGGGCACCAAGCTGGAAA |
| | | TCAAA |

Antibody B-H.1G LC-4

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3(Combined) | QQFTSSPFT |
| SEQ ID NO: 29 | VL | QNVLTQPPSASGTPGQRVTISCRASSSVNYIYWYQQLPGTA |
| | | PKLLIYYTSNLAPGVPDRFSGSGSGNSYSLAISGLRSEDEA |
| | | DYYCQQFTSSPFTFGTGTKVTVL |
| SEQ ID NO: 37 | DNA VL | CAGAATGTGCTGACCCAACCTCCTTCCGCCTCTGGCACA |
| | | CCTGGACAGAGAGTGACAATCTCCTGCCGGGCCTCCTCC |
| | | TCCGTGAACTACATCTACTGGTATCAGCAGCTGCCCGGC |
| | | ACCGCTCCTAAACTGCTGATCTACTACACCTCCAATCTG |
| | | GCCCCTGGCGTGCCCGATAGATTTTCCGGATCTGGCTCC |
| | | GGCAACTCCTACAGCCTGGCTATCTCTGGCCTGAGATCT |
| | | GAGGACGAGGCCGACTACTACTGCCAGCAGTTCACCTCC |
| | | TCTCCATTCACCTTTGGCACCGGCACCAAAGTGACAGTT |
| | | CTT |

Antibody B-H.1H LC-5

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3(Combined) | QQFTSSPFT |
| SEQ ID NO: | VL | SNELTQPPSVSVSPGQTARITCRASSSVNYIYWYQQKSGQA |
| 8673 | | PVLVIYYTSNLAPGIPERFSGSGSGNMYTLTISGAQVEDEA |
| | | DYYCQQFTSSPFTFGTGTKVTVL |
| SEQ ID NO: 38 | DNA VL | TCTAATGAGCTGACCCAGCCTCCTTCCGTGTCCGTGTCTC |
| | | CTGGACAGACCGCCAGAATTACCTGCCGGGCCTCCTCCT |
| | | CCGTGAACTACATCTACTGGTATCAGCAGAAGTCCGGCC |
| | | AGGCTCCTGTGCTCGTGATCTACTACACCTCCAATCTGGC |
| | | CCCTGGCATCCCTGAGAGATTCTCCGGATCTGGCTCCGG |
| | | CAACATGTACACCCTGACCATCTCTGGCGCCCAGGTGGA |
| | | AGATGAGGCCGACTACTACTGCCAGCAGTTCACCTCCTC |
| | | TCCATTCACCTTTGGCACCGGCACCAAAGTGACAGTTCT |
| | | T |

Antibody B-H.1

| | | |
|---|---|---|
| SEQ ID NO: | Chain 1: Fc only | METDTLLLWVLLLWVPGSTGDKTHTCPPCPAPELLGGPSV |
| 3280 | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPCREEMTK |
| | | NQVSLWCLVKGFYPSDIAVEWESNGQPENNYKTTPPVLDS |
| | | DGSFFLYSKLTVDKSRWQQGNVFSCSVMHEALHNRFTQKS |
| | | LSLSPGK |
| SEQ ID NO: | Chain 1: Fc only | METDTLLLWVLLLWVPGSTGDKTHTCPPCPAPELLGGPSV |
| 4693 | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPCREEMTK |
| | | NQVSLWCLVKGFYPSDIAVEWESNGQPENNYKTTPPVLDS |
| | | DGSFFLYSKLTVDKSRWQQGNVFSCSVMHEALHNRFTQKS |
| | | LSLSP |
| SEQ ID NO: | Chain 2: humanized B- | METDTLLLWVLLLWVPGSTGEVQLVESGGGLVQPGGSLRL |
| 3281 | H scFv | SCAASGFTFSNFGMHWVRQAPGKGLEWVSYISSGSSTIYYA |
| | | DTLKGRFTISRDNSKNTLYLQMNSLRAEDTAVYYCARRGE |
| | | GAMDYWGQGTTVTVSSGGGGSGGGGSGGGGSGGGGSDN |
| | | QLTQSPSFLSASVGDRVTITCRASSSVNYIYWYQQKPGKAP |
| | | KLLIYYTSNLAPGVPSRFSGSGSGNEYTLTISSLQPEDFATY |
| | | YCQQFTSSPFTFGQGTKLEIKGGGGSDKTHTCPPCPAPELLG |
| | | GPSVFLFPPKPKDTLMISRTPEVTCVVVDVSHEDPEVKFNW |
| | | YVDGVEVHNAKTKPREEQYNSTYRVVSVLTVLHQDWLNG |
| | | KEYKCKVSNKALPAPIEKTISKAKGQPREPQVCTLPPSREE |
| | | MTKNQVSLSCAVKGFYPSDIAVEWESNGQPENNYKTTPPV |
| | | LDSDGSFFLVSKLTVDKSRWQQGNVFSCSVMHEALHNHYT |
| | | QKSLSLSPGKGGGGSGGGGSGLNDIFEAQKIEWHE |
| SEQ ID NO: | scFv | EVQLVESGGGLVQPGGSLRLSCAASGFTFSNFGMHWVRQA |
| 3642 | | PGKGLEWVSYISSGSSTIYYADTLKGRFTISRDNSKNTLYLQ |
| | | MNSLRAEDTAVYYCARRGEGAMDYWGQGTTVTVSSGGG |
| | | GSGGGGSGGGGSGGGGSDNQLTQSPSFLSASVGDRVTITCR |
| | | ASSSVNYIYWYQQKPGKAPKLLIYYTSNLAPGVPSRFSGSG |
| | | SGNEYTLTISSLQPEDFATYYCQQFTSSPFTFGQGTKLEIK |

Antibody B-H.2

| | | |
|---|---|---|
| SEQ ID NO: | scFv | EVQLVESGGGLVQPGGSLRLSCAASGFTFSNFGMHWVRQA |
| 1338 | | PGKGLEWVSYISSGSSTIYYADTLKGRFTISRDNSKNTLYLQ |
| | | MNSLRAEDTAVYYCARRGEGAMDYWGQGTTVTVSSGGG |
| | | GSGGGGSGGGGSGGGGSDNQLTQSPSSLSASVGDRVTITCR |
| | | ASSSVNYIYWYQQKPGKAPKLLIYYTSNLAPGVPSRFSGSG |
| | | SGNDYTLTISSLQPEDFATYYCQQFTSSPFTFGQGTKLEIK |

Antibody B-H.3

| | | |
|---|---|---|
| SEQ ID NO: | scFv | EVQLVESGGGLVQPGGSLRLSCAASGFTFSNFGMHWVRQA |
| 1339 | | PGKGLEWVSYISSGSSTIYYADTLKGRFTISRDNSKNTLYLQ |
| | | MNSLRAEDTAVYYCARRGEGAMDYWGQGTTVTVSSGGG |
| | | GSGGGGSGGGGSGGGGSSNELTQPPSVSVSPGQTARITCRA |
| | | SSSVNYIYWYQQKSGQAPVLVIYYTSNLAPGIPERFSGSGSG |
| | | NMYTLTISGAQVEDEADYYCQQFTSSPFTFGTGTKVTVL |

Antibody B-H.4

| | | |
|---|---|---|
| SEQ ID NO: | scFv | QVQLVESGGGVVQPGRSLRLSCAASGFTFSNFGMHWVRQ |
| 1340 | | APGKGLEWVAYISSGSSTIYYADTLKGRFTISRDNSKNTLY |
| | | LQMNSLRAEDTAVYYCARRGEGAMDYWGQGTTVTVSSG |
| | | GGGSGGGGSGGGGSGGGGSDNQLTQSPSFLSASVGDRVTI |
| | | TCRASSSVNYIYWYQQKPGKAPKLLIYYTSNLAPGVPSRFS |
| | | GSGSGNEYTLTISSLQPEDFATYYCQQFTSSPFTFGQGTKLEI |
| | | K |

Antibody B-H.5

| | | |
|---|---|---|
| SEQ ID NO: | scFv | QVQLVESGGGVVQPGRSLRLSCAASGFTFSNFGMHWVRQ |
| 1341 | | APGKGLEWVAYISSGSSTIYYADTLKGRFTISRDNSKNTLY |
| | | LQMNSLRAEDTAVYYCARRGEGAMDYWGQGTTVTVSSG |
| | | GGGSGGGGSGGGGSGGGGSDNQLTQSPSSLSASVGDRVTI |
| | | TCRASSSVNYIYWYQQKPGKAPKLLIYYTSNLAPGVPSRFS |
| | | GSGSGNDYTLTISSLQPEDFATYYCQQFTSSPFTFGQGTKLE |
| | | IK |

Antibody B-H.6

| | | |
|---|---|---|
| SEQ ID NO: | scFv | QVQLVESGGGVVQPGRSLRLSCAASGFTFSNFGMHWVRQ |
| 1342 | | APGKGLEWVAYISSGSSTIYYADTLKGRFTISRDNSKNTLY |
| | | LQMNSLRAEDTAVYYCARRGEGAMDYWGQGTTVTVSSG |
| | | GGGSGGGGSGGGGSGGGGSSNELTQPPSVSVSPGQTARITC |
| | | RASSSVNYIYWYQQKSGQAPVLVIYYTSNLAPGIPERFSGS |
| | | GSGNMYTLTISGAQVEDEADYYCQQFTSSPFTFGTGTKVTV |
| | | L |

TABLE 39
Sequences of Knob-into-Hole designs of mono- and bi-specific antibodies (CDR sequences
in the amino acid chains are underlined)
1. Anti-TRBC2-NKp30-BIS-9 BKM0313 (TRBC2/NKp30 bispecific)

| | |
|---|---|
| Knob | Light Chain (LC) |
| chain | DVVMTQSPLSLPVTLGQPASISCRSSKNLVHSNGRTYLQWYQQRPGQSPRLLIYRVSNR |
| (TRBC2 | FPGVPDRFSGSGSGTDFTLKISRVEAEDVGVYFCSQSTREPYTFGGGTKVEIKRTVAAPS |
| Fab-Fc) | VFIFPPSDEQLKSGTASVVCLLNNFYPREAKVQWKVDNALQSGNSQESVTEQDSKDST |
| | YSLSSTLTLSKADYEKHKVYACEVTHQGLSSPVTKSFNRGEC (SEQ ID NO: 8281) |
| | LC Variable region: |
| | DVVMTQSPLSLPVTLGQPASISCCDRRSSKNLVHSNGRTYLQWYQQRPGQSPRLLIYRV |
| | SNRFPGVPDRFSGSGSGTDFTLKISRVEAEDVGVYFCSQSTREPYTFGGGTKVEIK (SEQ |
| | ID NO: 8282) |
| | LC-CDR1 sequence: |
| | RSSKNLVHSNGRTYLQ (SEQ ID NO: 8051) |
| | LC-CDR2 sequence: |
| | RVSNRFP (SEQ ID NO: 224) |
| | LC-CDR3 sequence: |
| | SQSTREPYT (SEQ ID NO: 8052) |
| | Heavy chain (HC): |
| | QVQLVQSGAEVKKPGSSVKVSCKASPRGFYGYHMHWVRQAPGQGLEWMGFINPYNN |
| | HIQYNERFRGRVTITSDESTTTAYMELSSLRSEDTAVYYCALGIGKWGDGAYRFFDFW |
| | GQGTLVTVSSASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHT |
| | CPPCPAPELLGGPSVFLFPPKPKDTLMISRTPEVTCVVVDVSHEDPEVKFNWYVDGVEV |
| | HNAKTKPREEQYASTYRVVSVLTVLHQDWLNGKEYKCKVSNKALPAPIEKTISKAKG |
| | QPREPQVYTLPPCREEMTKNQVSLWCLVKGFYPSDIAVEWESNGQPENNYKTTPPVLD |
| | SDGSFFLYSKLTVDKSRWQQGNVFSCSVMHEALHNHYTQKSLSLSPGK (SEQ ID NO: |
| | 8283) |
| | Heavy chain (HC): |
| | QVQLVQSGAEVKKPGSSVKVSCKASPRGFYGYHMHWVRQAPGQGLEWMGFINPYNN |
| | HIQYNERFRGRVTITSDESTTTAYMELSSLRSEDTAVYYCALGIGKWGDGAYRFFDFW |
| | GQGTLVTVSSASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHT |
| | CPPCPAPELLGGPSVFLFPPKPKDTLMISRTPEVTCVVVDVSHEDPEVKFNWYVDGVEV |
| | HNAKTKPREEQYASTYRVVSVLTVLHQDWLNGKEYKCKVSNKALPAPIEKTISKAKG |
| | QPREPQVYTLPPCREEMTKNQVSLWCLVKGFYPSDIAVEWESNGQPENNYKTTPPVLD |
| | SDGSFFLYSKLTVDK SR WQQGNVFSCSVMHEALHNHYTQKSLSLSP (SEQ ID NO: |
| | 4694) |
| | HC Variable region |
| | QVQLVQSGAEVKKPGSSVKVSCKASPRGFYGYHMHWVRQAPGQGLEWMGFINPYNN |
| | HIQYNERFRGRVTITSDESTTTAYMELSSLRSEDTAVYYCALGIGKWGDGAYRFFDFW |
| | GQGTLVTVSS (SEQ ID NO: 8284) |
| | HC-CDR1 |
| | PRGFYGYHMH (SEQ ID NO: 8272) |
| | HC-CDR2 |
| | FINPYNNHIQYNERFRG (SEQ ID NO: 8044) |
| | HC-CDR3 |
| | GIGKWGDGAYRFFDF (SEQ ID NO: 8285) |
| Hole | Full chain sequence |
| chain | EIQLLESGGGLVQPGGSLRLSCAVSGFSITTTGYHWNWVRQAPGKGLEWVGYIYSSGS |
| (NKp30 | TSYNPSLKSRFTISRDTSKNTFYLQMNSLRAEDTAVYYCARGDWHYFDYWGQGTMVT |
| scFv-Fc) | VSSGGGGSGGGGSGGGGSGGGGSDSVTTQSPLSLPVTLGQPASISCSGEKLSDKYVHW |
| | YQQRPGQSPRMLIYENDRRPSGVPDRFSGSNSGNDATLKISRVEAEDVGVYFCQFWDS |
| | TNSAVFGGGTKVEIKDKTHTCPPCPAPELLGGPSVFLFPPKPKDTLMISRTPEVTCVVVD |
| | VSHEDPEVKFNWYVDGVEVHNAKTKPREEQYASTYRVVSVLTVLHQDWLNGKEYKC |
| | KVSNKALPAPIEKTISKAKGQPREPQVCTLPPSREEMTKNQVSLSCAVKGFYPSDIAVE |
| | WESNGQPENNYKTTPPVLDSDGSFFLVSKLTVDKSRWQQGNVFSCSVMHEALHNHYT |
| | QKSLSLSPGK (SEQ ID NO: 8286) |
| | Full chain sequence |
| | EIQLLESGGGLVQPGGSLRLSCAVSGFSITTTGYHWNWVRQAPGKGLEWVGYIYSSGS |
| | TSYNPSLKSRFTISRDTSKNTFYLQMNSLRAEDTAVYYCARGDWHYFDYWGQGTMVT |
| | VSSGGGGSGGGGSGGGGSGGGGSDSVTTQSPLSLPVTLGQPASISCSGEKLSDKYVHW |
| | YQQRPGQSPRMLIYENDRRPSGVPDRFSGSNSGNDATLKISRVEAEDVGVYFCQFWDS |
| | TNSAVFGGGTKVEIKDKTHTCPPCPAPELLGGPSVFLFPPKPKDTLMISRTPEVTCVVVD |
| | VSHEDPEVKFNWYVDGVEVHNAKTKPREEQYASTYRVVSVLTVLHQDWLNGKEYKC |
| | KVSNKALPAPIEKTISKAKGQPREPQVCTLPPSREEMTKNQVSLSCAVKGFYPSDIAVE |
| | WESNGQPENNYKTTPPVLDSDGSFFLVSKLTVDKSRWQQGNVFSCSVMHEALHNHYT |
| | QKSLSLSP (SEQ ID NO: 4695) |
| | NKp30 scFv |
| | EIQLLESGGGLVQPGGSLRLSCAVSGFSITTTGYHWNWVRQAPGKGLEWVGYIYSSGS |
| | TSYNPSLKSRFTISRDTSKNTFYLQMNSLRAEDTAVYYCARGDWHYFDYWGQGTMVT |
| | VSSGGGGSGGGGSGGGGSGGGGSDSVTTQSPLSLPVTLGQPASISCSGEKLSDKYVHW |
| | YQQRPGQSPRMLIYENDRRPSGVPDRFSGSNSGNDATLKISRVEAEDVGVYFCQFWDS |
| | TNSAVFGGGTKVEIK (SEQ ID NO: 8287) |
| | Heavy chain sequence |
| | EIQLLESGGGLVQPGGSLRLSCAVSGFSITTTGYHWNWVRQAPGKGLEWVGYIYSSGS |
| | TSYNPSLKSRFTISRDTSKNTFYLQMNSLRAEDTAVYYCARGDWHYFDYWGQGTMVT |
| | VSS (SEQ ID NO: 8030) |
| | HC-CDR1: GFSITTTGYHWN (SEQ IS NO: 8288) |
| | HC-CDR2: YIYSSGSTSYNPSLKS (SEQ IS NO: 6001) |
| | HC-CDR3: GDWHYFDY (SEQ IS NO: 504) |
| | Light Chain sequence |
| | DSVTTQSPLSLPVTLGQPASISCSGEKLSDKYVHWYQQRPGQSPRMLIYENDRRPSGVP |
| | DRFSGSNSGNDATLKISRVEAEDVGVYFCQFWDSTNSAVFGGGTKVEIK (SEQ ID NO: |
| | 13) |
| | LC-CDR1: SGEKLSDKYVH (SEQ ID NO: 26) |
| | LC-CDR2: ENDRRPS (SEQ ID NO: 28) |
| | LC-CDR3: QFWDSTNSAV (SEQ ID NO: 86) |
| | Linker sequence: |
| | GGGGSGGGGSGGGGSGGGGS (SEQ ID NO: 8291) |

2. Anti-TRBC2-NKp30-BIS-10 BKM0314 (TRBC2/NKp30 bispecific)

| | |
|---|---|
| Knob | Light Chain (LC) |
| chain | DVVMTQSPLSLPVTLGQPASISCRSSKNLVHSNGRTYLQWYQQRPGQSPRLLIYRVSNR |
| (TRBC2 | FPGVPDRFSGSGSGTDFTLKISRVEAEDVGVYFCSQSTREPYTFGGGTKVEIKRTVAAPS |
| Fab-Fc) | VFIFPPSDEQLKSGTASVVCLLNNFYPREAKVQWKVDNALQSGNSQESVTEQDSKDST |
| | YSLSSTLTLSKADYEKHKVYACEVTHQGLSSPVTKSFNRGEC (SEQ ID NO: 8292) |
| | LC Variable region |
| | DVVMTQSPLSLPVTLGQPASISCRSSKNLVHSNGRTYLQWYQQRPGQSPRLLIYRVSNR |
| | FPGVPDRFSGSGSGTDFTLKISRVEAEDVGVYFCSQSTREPYTFGGGTKVEIK (SEQ ID |
| | NO: 8014) |
| | LC-CDR1 |
| | RSSKNLVHSNGRTYLQ (SEQ ID NO: 8051) |
| | LC-CDR2 |
| | RVSNRFP (SEQ ID NO: 224) |
| | LC-CDR3 |
| | SQSTREPYT (SEQ ID NO: 8052) |
| | Heavy chain (HC): |
| | QVQLVQSGAEVKKPGSSVKVSCKASPRGFYGYHMHWVRQAPGQGLEWMGFINPYNN |
| | HIQYNERFRGRVTITSDESTTTAYMELSSLRSEDTAVYYCALGIGKWGDGAYRFFDFW |
| | GQGTLVTVSSASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHT |
| | CPPCPAPELLGGPSVFLFPPKPKDTLMISRTPEVTCVVVDVSHEDPEVKFNWYVDGVEV |
| | HNAKTKPREEQYASTYRVVSVLTVLHQDWLNGKEYKCKVSNKALPAPIEKTISKAKG |
| | QPREPQVYTLPPCREEMTKNQVSLWCLVKGFYPSDIAVEWESNGQPENNYKTTPPVLD |
| | SDGSFFLYSKLTVDKSRWQQGNVFSCSVMHEALHNHYTQKSLSLSPGK (SEQ ID NO: |
| | 8294) |
| | Heavy chain (HC): |
| | QVQLVQSGAEVKKPGSSVKVSCKASPRGFYGYHMHWVRQAPGQGLEWMGFINPYNN |
| | HIQYNERFRGRVTITSDESTTTAYMELSSLRSEDTAVYYCALGIGKWGDGAYRFFDFW |
| | GQGTLVTVSSASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHT |
| | CPPCPAPELLGGPSVFLFPPKPKDTLMISRTPEVTCVVVDVSHEDPEVKFNWYVDGVEV |
| | HNAKTKPREEQYASTYRVVSVLTVLHQDWLNGKEYKCKVSNKALPAPIEKTISKAKG |
| | QPREPQVYTLPPCREEMTKNQVSLWCLVKGFYPSDIAVEWESNGQPENNYKTTPPVLD |
| | SDGSFFLYSKLTVDKSRWQQGNVFSCSVMHEALHNHYTQKSLSLSP (SEQ ID NO: |
| | 4696) |
| | HC-Variable region |
| | QVQLVQSGAEVKKPGSSVKVSCKASPRGFYGYHMHWVRQAPGQGLEWMGFINPYNN |
| | HIQYNERFRGRVTITSDESTTTAYMELSSLRSEDTAVYYCALGIGKWGDGAYRFFDFW |
| | GQGTLVTVSS (SEQ ID NO: 8295) |
| | HC-CDR1 |
| | PRGFYGYHMH (SEQ ID NO: 8272) |
| | HC-CDR2 |
| | FINPYNNHIQYNERFRG (SEQ ID NO: 8044) |
| | HC-CDR3 |
| | GVGKWGDGAYRFFDF (SEQ ID NO: 8296) |
| Hole | EIQLLESGGGLVQPGGSLRLSCAVSGFSITTTGYHWNWVRQAPGKGLEWVGYIYSSGS |
| chain | TSYNPSLKSRFTISRDTSKNTFYLQMNSLRAEDTAVYYCARGDWHYFDYWGQGTMVT |
| (Nkp30 | VSSGGGGSGGGGSGGGGSGGGGSDSVTTQSPLSLPVTLGQPASISCSGEKLSDKYVH |
| scFv-Fc) | WYQQRPGQSPRMLIYENDRRPSGVPDRFSGSNSGNDATLKISRVEAEDVGVYFCQFWD |
| | STNSAVFGGGTKVEIKDKTHTCPPCPAPELLGGPSVFLFPPKPKDTLMISRTPEVTCVVV |
| | DVSHEDPEVKFNWYVDGVEVHNAKTKPREEQYASTYRVVSVLTVLHQDWLNGKEYK |
| | CKVSNKALPAPIEKTISKAKGQPREPQVCTLPPSREEMTKNQVSLSCAVKGFYPSDIAVE |
| | WESNGQPENNYKTTPPVLDSDGSFFLVSKLTVDKSRWQQGNVFSCSVMHEALHNHYT |
| | QKSLSLSPGK (SEQ ID NO: 8286) |
| | EIQLLESGGGLVQPGGSLRLSCAVSGFSITTTGYHWNWVRQAPGKGLEWVGYIYSSGS |
| | TSYNPSLKSRFTISRDTSKNTFYLQMNSLRAEDTAVYYCARGDWHYFDYWGQGTMVT |
| | VSSGGGGSGGGGSGGGGSGGGGSDSVTTQSPLSLPVTLGQPASISCSGEKLSDKYVH |
| | WYQQRPGQSPRMLIYENDRRPSGVPDRFSGSNSGNDATLKISRVEAEDVGVYFCQFWD |
| | STNSAVFGGGTKVEIKDKTHTCPPCPAPELLGGPSVFLFPPKPKDTLMISRTPEVTCVVV |
| | DVSHEDPEVKFNWYVDGVEVHNAKTKPREEQYASTYRVVSVLTVLHQDWLNGKEYK |
| | CKVSNKALPAPIEKTISKAKGQPREPQVCTLPPSREEMTKNQVSLSCAVKGFYPSDIAVE |
| | WESNGQPENNYKTTPPVLDSDGSFFLVSKLTVDKSRWQQGNVFSCSVMHEALHNHYT |
| | QKSLSLSP (SEQ ID NO: 4698) |
| | NKp30 ScFv |
| | EIQLLESGGGLVQPGGSLRLSCAVSGFSITTTGYHWNWVRQAPGKGLEWVGYIYSSGS |
| | TSYNPSLKSRFTISRDTSKNTFYLQMNSLRAEDTAVYYCARGDWHYFDYWGQGTMVT |
| | VSSGGGGSGGGGSGGGGSGGGGSDSVTTQSPLSLPVTLGQPASISCSGEKLSDKYVH |
| | WYQQRPGQSPRMLIYENDRRPSGVPDRFSGSNSGNDATLKISRVEAEDVGVYFCQFWDS |
| | TNSAVFGGGTKVEIK (SEQ ID NO: 8287) |
| | Heavy chain sequence |
| | EIQLLESGGGLVQPGGSLRLSCAVSGFSITTTGYHWNWVRQAPGKGLEWVGYIYSSGS |
| | TSYNPSLKSRFTISRDTSKNTFYLQMNSLRAEDTAVYYCARGDWHYFDYWGQGTMVT |
| | VSS (SEQ ID NO: 8030) |
| | Light chain sequence |
| | DSVTTQSPLSLPVTLGQPASISCSGEKLSDKYVHWYQQRPGQSPRMLIYENDRRPSGVP |
| | DRFSGSNSGNDATLKISRVEAEDVGVYFCQFWDSTNSAVFGGGTKVEIK (SEQ ID NO: |
| | 13) |
| | Linker sequence |
| | GGGGSGGGGSGGGGSGGGGS (SEQ ID NO: 8291) |

3. Anti-TRBC2-M11 BKM0343 (TRBC2 monovalent mAb)

| | |
|---|---|
| Knob | Light chain sequence |
| chain | DVVMTQSPLSLPVTLGQPASISCRSSKNLVHSNGRTYLQWYQQRPGQSPRLLIYRVSNR |
| (TRBC2 | FPGVPDRFSGSGSGTDFTLKISRVEAEDVGVYFCSQSTREPYTFGGGTKVEIKRTVAAPS |
| Fab-Fc) | VFIFPPSDEQLKSGTASVVCLLNNFYPREAKVQWKVDNALQSGNSQESVTEQDSKDST |
| | YSLSSTLTLSKADYEKHKVYACEVTHQGLSSPVTKSFNRGEC (SEQ ID NO: 8297) |
| | Variable region |
| | DVVMTQSPLSLPVTLGQPASISCRSSKNLVHSNGRTYLQWYQQRPGQSPRLLIYRVSNR |
| | FPGVPDRFSGSGSGTDFTLKISRVEAEDVGVYFCSQSTREPYTFGGGTKVEIK (SEQ ID |
| | NO: 8014) |
| | LC-CDR1 |
| | RSSKNLVHSNGRTYLQ (SEQ ID NO: 8051) |
| | LC-CDR2 |
| | RVSNRFP (SEQ ID NO: 224) |
| | LC-CDR3 |
| | SQSTREPYT (SEQ ID NO: 8052) |
| | Heavy chain sequence |
| | QVQLVQSGAEVKKPGSSVKVSCKASPRGFYGYHMHWVRQAPGQGLEWMGFINPYNN |
| | HIQYNERFRGRVTITSDESTTTAYMELSSLRSEDTAVYYCALGIGKWGDGAYRFFDFW |
| | GQGTLVTVSSASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHT |
| | CPPCPAPELLGGPSVFLFPPKPKDTLMISRTPEVTCVVVDVSHEDPEVKFNWYVDGVEV |
| | HNAKTKPREEQYASTYRVVSVLTVLHQDWLNGKEYKCKVSNKALPAPIEKTISKAKG |
| | QPREPQVYTLPPCREEMTKNQVSLWCLVKGFYPSDIAVEWESNGQPENNYKTTPPVLD |
| | SDGSFFLYSKLTVDKSRWQQGNVFSCSVMHEALHNHYTQKSLSLSPGK (SEQ ID NO: |
| | 8298) |
| | Heavy chain sequence |
| | QVQLVQSGAEVKKPGSSVKVSCKASPRGFYGYHMHWVRQAPGQGLEWMGFINPYNN |
| | HIQYNERFRGRVTITSDESTTTAYMELSSLRSEDTAVYYCALGIGKWGDGAYRFFDFW |
| | GQGTLVTVSSASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHT |
| | CPPCPAPELLGGPSVFLFPPKPKDTLMISRTPEVTCVVVDVSHEDPEVKFNWYVDGVEV |
| | HNAKTKPREEQYASTYRVVSVLTVLHQDWLNGKEYKCKVSNKALPAPIEKTISKAKG |
| | QPREPQVYTLPPCREEMTKNQVSLWCLVKGFYPSDIAVEWESNGQPENNYKTTPPVLD |
| | SDGSFFLYSKLTVDKSRWQQGNVFSCSVMHEALHNHYTQKSLSLSP (SEQ ID NO: |
| | 4699) |
| | Variable region |
| | QVQLVQSGAEVKKPGSSVKVSCKASPRGFYGYHMHWVRQAPGQGLEWMGFINPYNN |
| | HIQYNERFRGRVTITSDESTTTAYMELSSLRSEDTAVYYCALGIGKWGDGAYRFFDFW |
| | GQGTLVTVSS (SEQ ID NO: 8299) |
| | HC-CDR1 |
| | PRGFYGYHMH (SEQ ID NO: 8272) |
| | HC-CDR2 |
| | FINPYNNHIQYNERFRG (SEQ ID NO: 8044) |
| | HC-CDR3 |
| | GEGKWGDGAYRFFDE (SEQ ID NO: 8046) |
| Hole | DKTHTCPPCPAPELLGGPSVFLFPPKPKDTLMISRTPEVTCVVVDVSHEDPEVKFNWYV |
| chain | DGVEVHNAKTKPREEQYASTYRVVSVLTVLHQDWLNGKEYKCKVSNKALPAPIEKTI |
| (Fc) | SKAKGQPREPQVCTLPPSREEMTKNQVSLSCAVKGFYPSDIAVEWESNGQPENNYKTT |
| | PPVLDSDGSFFLVSKLTVDKSRWQQGNVFSCSVMHEALHNHYTQKSLSLSPGK (SEQ |
| | ID NO: 3477) |

4. Anti-TRBC2-M12 BKM0344 (TRBC2 monovalent mAb)

| | |
|---|---|
| Knob | Light Chain |
| chain | DVVMTQSPLSLPVTLGQPASISCRSSKNLVHSNGRTYLQWYQQRPGQSPRLLIYRVSNR |
| (TRBC2 | FPGVPDRFSGSGSGTDFTLKISRVEAEDVGVYFCSQSTREPYTFGGGTKVEIKRTVAAPS |
| Fab-Fc) | VFIFPPSDEQLKSGTASVVCLLNNFYPREAKVQWKVDNALQSGNSQESVTEQDSKDST |
| | YSLSSTLTLSKADYEKHKVYACEVTHQGLSSPVTKSFNRGEC (SEQ ID NO: 8301) |
| | Light chain variable region |
| | DVVMTQSPLSLPVTLGQPASISCRSSKNLVHSNGRTYLQWYQQRPGQSPRLLIYRVSNR |
| | FPGVPDRFSGSGSGTDFTLKISRVEAEDVGVYFCSQSTREPYTFGGGTKVEIK (SEQ ID |
| | NO: 8014) |
| | LC-CDR1 |
| | RSSKNLVHSNGRTYLQ (SEQ ID NO: 8051) |
| | LC-CDR2 |
| | RVSNRFP (SEQ ID NO: 224) |
| | LC-CDR3 |
| | SQSTREPYT (SEQ ID NO: 8052) |
| | Heavy Chain |
| | QVQLVQSGAEVKKPGSSVKVSCKASPRGFYGYHMHWVRQAPGQGLEWMGFINPYNN |
| | HIQYNERFRGRVTITSDESTTTAYMELSSLRSEDTAVYYCALGIGKWGDGAYRFFDFW |
| | GQGTLVTVSSASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHT |
| | CPPCPAPELLGGPSVFLFPPKPKDTLMISRTPEVTCVVVDVSHEDPEVKFNWYVDGVEV |
| | HNAKTKPREEQYASTYRVVSVLTVLHQDWLNGKEYKCKVSNKALPAPIEKTISKAKG |
| | QPREPQVYTLPPCREEMTKNQVSLWCLVKGFYPSDIAVEWESNGQPENNYKTTPPVLD |
| | SDGSFFLYSKLTVDKSRWQQGNVFSCSVMHEALHNHYTQKSLSLSPGK (SEQ ID NO: |
| | 8302) |
| | Heavy Chain |
| | QVQLVQSGAEVKKPGSSVKVSCKASPRGFYGYHMHWVRQAPGQGLEWMGFINPYNN |
| | HIQYNERFRGRVTITSDESTTTAYMELSSLRSEDTAVYYCALGIGKWGDGAYRFFDFW |
| | GQGTLVTVSSASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHT |
| | CPPCPAPELLGGPSVFLFPPKPKDTLMISRTPEVTCVVVDVSHEDPEVKFNWYVDGVEV |
| | HNAKTKPREEQYASTYRVVSVLTVLHQDWLNGKEYKCKVSNKALPAPIEKTISKAKG |
| | QPREPQVYTLPPCREEMTKNQVSLWCLVKGFYPSDIAVEWESNGQPENNYKTTPPVLD |
| | SDGSFFLYSKLTVDKSR WQQGNVFSCSVMHEALHNHYTQKSLSLSP (SEQ ID NO: |
| | 4700) |
| | Variable region |
| | QVQLVQSGAEVKKPGSSVKVSCKASPRGFYGYHMHWVRQAPGQGLEWMGFINPYNN |
| | HIQYNERFRGRVTITSDESTTTAYMELSSLRSEDTAVYYCALGIGKWGDGAYRFFDFW |
| | GQGTLVTVSS (SEQ ID NO: 8303) |
| | HC-CDR1 |
| | PRGFYGYHMH (SEQ ID NO: 8272) |
| | HC-CDR2 |
| | FINPYNNHIQYNERFRG (SEQ ID NO: 8044) |
| | HC-CDR3 |
| | GAGKWGDGAYRFFDF (SEQ ID NO: 8047) |
| Hole | DKTHTCPPCPAPELLGGPSVFLFPPKPKDTLMISRTPEVTCVVVDVSHEDPEVKFNWYV |
| chain | DGVEVHNAKTKPREEQYASTYRVVSVLTVLHQDWLNGKEYKCKVSNKALPAPIEKTI |
| (Fc) | SKAKGQPREPQVCTLPPSREEMTKNQVSLSCAVKGFYPSDIAVEWESNGQPENNYKTT |
| | PPVLDSDGSFFLVSKLTVDKSRWQQGNVFSCSVMHEALHNHYTQKSLSLSPGK (SEQ |
| | ID NO: 3477) |

5. Anti-TRBC1-NKp30-BIS-13 BJM1042 (TRBC1/NKp30 bispecific)

| | |
|---|---|
| Knob | Light Chain |
| chain | DVVMTQSPLSLPVTLGQPASISCRSSQRLVHSNGNTYLHWYQQRPGQSPRLLIYRVSNR |
| (TRBC1 | FPGVPDRFSGSGSGTDFTLKISRVEAEDVGVYFCSQSTHVPYTFGGGTKVEIKRTVAAP |
| Fab-Fc) | SVFIFPPSDEQLKSGTASVVCLLNNFYPREAKVQWKVDNALQSGNSQESVTEQDSKDS |
| | TYSLSSTLTLSKADYEKHKVYACEVTHQGLSSPVTKSFNRGEC (SEQ ID NO: 7380) |
| | Light chain variable region |
| | DVVMTQSPLSLPVTLGQPASISCRSSQRLVHSNGNTYLHWYQQRPGQSPRLLIYRVSNR |
| | FPGVPDRFSGSGSGTDFTLKISRVEAEDVGVYFCSQSTHVPYTFGGGTKVEIK (SEQ ID |
| | NO: 258) |
| | LC-CDR1 |
| | RSSQRLVHSNGNTYLH (SEQ ID NO: 223) |
| | LC-CDR2 |
| | RVSNRFP (SEQ ID NO: 224) |
| | LC-CDR3 |
| | SQSTHVPYT (SEQ ID NO: 225) |
| | Heavy chain |
| | QVQLVQSGAEVKKPGSSVKVSCKASGYTFTGYVMHWVRQAPGQGLEWMGFIIPIFGT |
| | ANYAQKFQGRVTITSDKSTTTAYMELSSLRSEDTAVYYCARGAGYNFDGAYRFFDFW |
| | GQGTLVTVSSASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHT |
| | CPPCPAPELLGGPSVFLFPPKPKDTLMISRTPEVTCVVVDVSHEDPEVKFNWYVDGVEV |
| | HNAKTKPREEQYASTYRVVSVLTVLHQDWLNGKEYKCKVSNKALPAPIEKTISKAKG |
| | QPREPQVYTLPPCREEMTKNQVSLWCLVKGFYPSDIAVEWESNGQPENNYKTTPPVLD |
| | SDGSFFLYSKLTVDKSRWQQGNVFSCSVMHEALHNHYTQKSLSLSPGK (SEQ ID NO: |
| | 7382) |
| | Heavy chain |
| | QVQLVQSGAEVKKPGSSVKVSCKASGYTFTGYVMHWVRQAPGQGLEWMGFIIPIFGT |
| | ANYAQKFQGRVTITSDKSTTTAYMELSSLRSEDTAVYYCARGAGYNFDGAYRFFDFW |
| | GQGTLVTVSSASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHT |
| | CPPCPAPELLGGPSVFLFPPKPKDTLMISRTPEVTCVVVDVSHEDPEVKFNWYVDGVEV |
| | HNAKTKPREEQYASTYRVVSVLTVLHQDWLNGKEYKCKVSNKALPAPIEKTISKAKG |
| | QPREPQVYTLPPCREEMTKNQVSLWCLVKGFYPSDIAVEWESNGQPENNYKTTPPVLD |
| | SDGSFFLYSKLTVDKSR WQQGNVFSCSVMHEALHNHYTQKSLSLSP (SEQ ID NO: |
| | 4677) |
| | HC-variable region |
| | QVQLVQSGAEVKKPGSSVKVSCKASGYTFTGYVMHWVRQAPGQGLEWMGFIIPIFGT |
| | ANYAQKFQGRVTITSDKSTTTAYMELSSLRSEDTAVYYCARGAGYNFDGAYRFFDFW |
| | GQGTLVTVSS (SEQ ID NO: 8304) |
| | HC-CDR1 |
| | GYTFTGYVMH (SEQ ID NO: 8305) |
| | HC-CDR2 |
| | FIIPIFGTANYAQKFQG (SEQ ID NO: 7355), |
| | HC-CDR3 |
| | GAGYNFDGAYRFFDF (SEQ ID NO: 202) |
| Hole | EIQLLESGGGLVQPGGSLRLSCAVSGFSITTTGYHWNWVRQAPGKGLEWVGYIYSSGS |
| chain | TSYNPSLKSRFTISRDTSKNTFYLQMNSLRAEDTAVYYCARGDWHYFDYWGQGTMVT |
| (NKp30 | VSSGGGGGGGGSGGGGSGGGGSDSVTTQSPLSLPVTLGQPASISCSGEKLSDKYVHW |
| scFv-Fc) | YQQRPGQSPRMLIYENDRRPSGVPDRFSGSNSGNDATLKISRVEAEDVGVYFCQFWDS |
| | TNSAVFGGGTKVEIKDKTHTCPPCPAPELLGGPSVFLFPPKPKDTLMISRTPEVTCVVVD |
| | VSHEDPEVKFNWYVDGVEVHNAKTKPREEQYASTYRVVSVLTVLHQDWLNGKEYKC |
| | KVSNKALPAPIEKTISKAKGQPREPQVCTLPPSREEMTKNQVSLSCAVKGFYPSDIAVE |
| | WESNGQPENNYKTTPPVLDSDGSFFLVSKLTVDKSRWQQGNVFSCSVMHEALHNHYT |
| | QKSLSLSPGK (SEQ ID NO: 8286) |
| | EIQLLESGGGLVQPGGSLRLSCAVSGFSITTTGYHWNWVRQAPGKGLEWVGYIYSSGS |
| | TSYNPSLKSRFTISRDTSKNTFYLQMNSLRAEDTAVYYCARGDWHYFDYWGQGTMVT |
| | VSSGGGGSGGGGSGGGGSGGGGSDSVTTQSPLSLPVTLGQPASISCSGEKLSDKYVHW |
| | YQQRPGQSPRMLIYENDRRPSGVPDRFSGSNSGNDATLKISRVEAEDVGVYFCQFWDS |
| | TNSAVFGGGTKVEIKDKTHTCPPCPAPELLGGPSVFLFPPKPKDTLMISRTPEVTCVVVD |
| | VSHEDPEVKFNWYVDGVEVHNAKTKPREEQYASTYRVVSVLTVLHQDWLNGKEYKC |
| | KVSNKALPAPIEKTISKAKGQPREPQVCTLPPSREEMTKNQVSLSCAVKGFYPSDIAVE |
| | WESNGQPENNYKTTPPVLDSDGSFFLVSKLTVDKSRWQQGNVFSCSVMHEALHNHYT |
| | QKSLSLSP (SEQ ID NO: 4695) |
| | Variable region |
| | EIQLLESGGGLVQPGGSLRLSCAVSGFSITTTGYHWNWVRQAPGKGLEWVGYIYSSGS |
| | TSYNPSLKSRFTISRDTSKNTFYLQMNSLRAEDTAVYYCARGDWHYFDYWGQGTMVT |
| | VSSGGGGSGGGGSGGGGSGGGGSDSVTTQSPLSLPVTLGQPASISCSGEKLSDKYVHW |
| | YQQRPGQSPRMLIYENDRRPSGVPDRFSGSNSGNDATLKISRVEAEDVGVYFCQFWDS |
| | TNSAVFGGGTKVEIK (SEQ ID NO: 8287) |
| | Heavy chain sequence |
| | EIQLLESGGGLVQPGGSLRLSCAVSGFSITTTGYHWNWVRQAPGKGLEWVGYIYSSGS |
| | TSYNPSLKSRFTISRDTSKNTFYLQMNSLRAEDTAVYYCARGDWHYFDYWGQGTMVT |
| | VSS (SEQ ID NO: 8030) |
| | Light chain sequence |
| | DSVTTQSPLSLPVTLGQPASISCSGEKLSDKYVHWYQQRPGQSPRMLIYENDRRPSGVP |
| | DRFSGSNSGNDATLKISRVEAEDVGVYFCQFWDSTNSAVFGGGTKVEIK (SEQ ID NO: |
| | 13) |
| | Linker |
| | GGGGSGGGGSGGGGSGGGGS (SEQ ID NO: 8291) |

6. Anti-NKp30-M-14 BJM1077 (monovalent NKp30 scFv-Fc)
(monovalent control NKp30 binder only)

| | |
|---|---|
| Knob | DKTHTCPPCPAPELLGGPSVFLFPPKPKDTLMISRTPEVTCVVVDVSHEDPEVKFNWYV |
| chain | DGVEVHNAKTKPREEQYASTYRVVSVLTVLHQDWLNGKEYKCKVSNKALPAPIEKTI |
| (Fc) | SKAKGQPREPQVYTLPPCREEMTKNQVSLWCLVKGFYPSDIAVEWESNGQPENNYKT |
| | TPPVLDSDGSFFLYSKLTVDKSRWQQGNVFSCSVMHEALHNHYTQKSLSLSPGK (SEQ |
| | ID NO: 8306) |
| Hole | EIQLLESGGGLVQPGGSLRLSCAVSGFSITTTGYHWNWVRQAPGKGLEWVGYIYSSGS |
| Chain | TSYNPSLKSRFTISRDTSKNTFYLQMNSLRAEDTAVYYCARGDWHYFDYWGQGTMVT |
| (NKp30 | VSSGGGGSGGGGSGGGGSGGGGSDSVTTQSPLSLPVTLGQPASISCSGEKLSDKYVHW |
| scFv-Fc) | YQQRPGQSPRMLIYENDRRPSGVPDRFSGSNSGNDATLKISRVEAEDVGVYFCQFWDS |
| | TNSAVFGGGTKVEIKDKTHTCPPCPAPELLGGPSVFLFPPKPKDTLMISRTPEVTCVVVD |
| | VSHEDPEVKFNWYVDGVEVHNAKTKPREEQYASTYRVVSVLTVLHQDWLNGKEYKC |
| | KVSNKALPAPIEKTISKAKGQPREPQVCTLPPSREEMTKNQVSLSCAVKGFYPSDIAVE |
| | WESNGQPENNYKTTPPVLDSDGSFFLVSKLTVDKSRWQQGNVFSCSVMHEALHNHYT |
| | QKSLSLSPGK (SEQ ID NO: 8286) |
| | EIQLLESGGGLVQPGGSLRLSCAVSGFSITTTGYHWNWVRQAPGKGLEWVGYIYSSGS |
| | TSYNPSLKSRFTISRDTSKNTFYLQMNSLRAEDTAVYYCARGDWHYFDYWGQGTMVT |
| | VSSGGGGSGGGGSGGGGSGGGGSDSVTTQSPLSLPVTLGQPASISCSGEKLSDKYVHW |
| | YQQRPGQSPRMLIYENDRRPSGVPDRFSGSNSGNDATLKISRVEAEDVGVYFCQFWDS |
| | TNSAVFGGGTKVEIKDKTHTCPPCPAPELLGGPSVFLFPPKPKDTLMISRTPEVTCVVVD |
| | VSHEDPEVKFNWYVDGVEVHNAKTKPREEQYASTYRVVSVLTVLHQDWLNGKEYKC |
| | KVSNKALPAPIEKTISKAKGQPREPQVCTLPPSREEMTKNQVSLSCAVKGFYPSDIAVE |
| | WESNGQPENNYKTTPPVLDSDGSFFLVSKLTVDKSRWQQGNVFSCSVMHEALHNHYT |
| | QKSLSLSP (SEQ ID NO: 4695) |
| | NKp30 ScFv |
| | EIQLLESGGGLVQPGGSLRLSCAVSGFSITTTGYHWNWVRQAPGKGLEWVGYIYSSGS |
| | TSYNPSLKSRFTISRDTSKNTFYLQMNSLRAEDTAVYYCARGDWHYFDYWGQGTMVT |
| | VSSGGGGSGGGGSGGGGSGGGGSDSVTTQSPLSLPVTLGQPASISCSGEKLSDKYVHW |
| | YQQRPGQSPRMLIYENDRRPSGVPDRFSGSNSGNDATLKISRVEAEDVGVYFCQFWDS |
| | TNSAVFGGGTKVEIK (SEQ ID NO: 8287) |
| | Heavy chain sequence |
| | EIQLLESGGGLVQPGGSLRLSCAVSGFSITTTGYHWNWVRQAPGKGLEWVGYIYSSGS |
| | TSYNPSLKSRFTISRDTSKNTFYLQMNSLRAEDTAVYYCARGDWHYFDYWGQGTMVT |
| | VSS (SEQ ID NO: 8030) |
| | Light chain sequence |
| | DSVTTQSPLSLPVTLGQPASISCSGEKLSDKYVHWYQQRPGQSPRMLIYENDRRPSGVP |
| | DRFSGSNSGNDATLKISRVEAEDVGVYFCQFWDSTNSAVFGGGTKVEIK (SEQ ID NO: |
| | 13) |
| | GGGGGGGGSGGGGSGGGGS (SEQ ID NO: 8291) |

7. Anti-TRBC1-M15 BJM1083 (TRBC1 monovalent mAb)

| | |
|---|---|
| Knob | Light Chain (LC) |
| chain | DVVMTQSPLSLPVTLGQPASISCRSSQRLVHSNGNTYLHWYQQRPGQSPRLLIYRVSNR |
| (TRBC1 | FPGVPDRFSGSGSGTDFTLKISRVEAEDVGVYFCSQSTHVPYTFGGGTKVEIKRTVAAP |
| Fab-Fc) | SVFIFPPSDEQLKSGTASVVCLLNNFYPREAKVQWKVDNALQSGNSQESVTEQDSKDS |
| | TYSLSSTLTLSKADYEKHKVYACEVTHQGLSSPVTKSFNRGEC (SEQ ID NO: 8307) |
| | LC-Variable region |
| | DVVMTQSPLSLPVTLGQPASISCRSSQRLVHSNGNTYLHWYQQRPGQSPRLLIYRVSNR |
| | FPGVPDRFSGSGSGTDFTLKISRVEAEDVGVYFCSQSTHVPYTFGGGTKVEIK (SEQ ID |
| | NO: 8308) |
| | LC-CDR1 |
| | RSSQRLVHSNGNTYLH (SEQ ID NO: 223) |
| | LC-CDR2 |
| | RVSNRFP (SEQ ID NO: 224) |
| | LC-CDR3 |
| | SQSTHVPYT (SEQ ID NO: 225) |
| | Heavy Chain (HC) |
| | QVQLVQSGAEVKKPGSSVKVSCKASGYTFTGYVMHWVRQAPGQGLEWMGFIIPIFGT |
| | ANYAQKFQGRVTITSDKSTTTAYMELSSLRSEDTAVYYCARGAGYNFDGAYRFFDFW |
| | GQGTLVTVSSASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHT |
| | CPPCPAPELLGGPSVFLFPPKPKDTLMISRTPEVTCVVVDVSHEDPEVKFNWYVDGVEV |
| | HNAKTKPREEQYASTYRVVSVLTVLHQDWLNGKEYKCKVSNKALPAPIEKTISKAKG |
| | QPREPQVYTLPPCREEMTKNQVSLWCLVKGFYPSDIAVEWESNGQPENNYKTTPPVLD |
| | SDGSFFLYSKLTVDKSRWQQGNVFSCSVMHEALHNHYTQKSLSLSPGK (SEQ ID NO: |
| | 8309) |
| | Heavy Chain (HC) |
| | QVQLVQSGAEVKKPGSSVKVSCKASGYTFTGYVMHWVRQAPGQGLEWMGFIIPIFGT |
| | ANYAQKFQGRVTITSDKSTTTAYMELSSLRSEDTAVYYCARGAGYNFDGAYRFFDFW |
| | GQGTLVTVSSASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHT |
| | CPPCPAPELLGGPSVFLFPPKPKDTLMISRTPEVTCVVVDVSHEDPEVKFNWYVDGVEV |
| | HNAKTKPREEQYASTYRVVSVLTVLHQDWLNGKEYKCKVSNKALPAPIEKTISKAKG |
| | QPREPQVYTLPPCREEMTKNQVSLWCLVKGFYPSDIAVEWESNGQPENNYKTTPPVLD |
| | SDGSFFLYSKLTVDKSRWQQGNVFSCSVMHEALHNHYTQKSLSLSP (SEQ ID NO: 4701) |
| | Variable region |
| | QVQLVQSGAEVKKPGSSVKVSCKASGYTFTGYVMHWVRQAPGQGLEWMGFIIPIFGT |
| | ANYAQKFQGRVTITSDKSTTTAYMELSSLRSEDTAVYYCARGAGYNFDGAYRFFDFW |
| | GQGTLVTVSS (SEQ ID NO: 8310) |
| | HC-CDR1 |
| | GYTFTGYVMH (SEQ ID NO: 8305) |
| | HC-CDR2 |
| | FIIPIFGTANYAQKFQG (SEQ ID NO: 7355) |
| | HC-CDR3 |
| | GAGYNFDGAYRFFDF (SEQ ID NO: 202) |
| Hole | DKTHTCPPCPAPELLGGPSVFLFPPKPKDTLMISRTPEVTCVVVDVSHEDPEVKFNWYV |
| chain | DGVEVHNAKTKPREEQYASTYRVVSVLTVLHQDWLNGKEYKCKVSNKALPAPIEKTI |
| (Fc) | SKAKGQPREPQVCTLPPSREEMTKNQVSLSCAVKGFYPSDIAVEWESNGQPENNYKTT |
| | PPVLDSDGSFFLVSKLTVDKSRWQQGNVFSCSVMHEALHNHYTQKSLSLSPGK (SEQ |
| | ID NO: 3477) |

TABLE 40
Exemplary molecule constructs
Exemplary molecule 1

| | | |
|---|---|---|
| SEQ ID | Heavy chain | EIQLLESGGGLVQPGGSLRLSCAVSGFSIIRVGYHWNWVRQAPGKGLEWVG |
| NO: 500 | (VH-CH)- | YIHRSGSTSYNPSLKSRFTISRDTSKNTFYLQMNSLRAEDTAVYYCARGDWH |
| | Hole | YFDYWGQGTMVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNHK |
| | | PSNTKVDKRVEPKSCDKTHTCPPCPAPELLGGPSVFLFPPKPKDTLMISRTPE |
| | | VTCVVVDVSHEDPEVKFNWYVDGVEVHNAKTKPREEQYASTYRVVSVLTV |
| | | LHQDWLNGKEYKCKVSNKALPAPIEKTISKAKGQPREPQVCTLPPSREEMTK |
| | | NQVSLSCAVKGFYPSDIAVEWESNGQPENNYKTTPPVLDSDGSFFLVSKLTV |
| | | DKSRWQQGNVFSCSVMHEALHNHYTQKSLSLSPGK |
| SEQ ID | Heavy chain | EIQLLESGGGLVQPGGSLRLSCAVSGFSIIRVGYHWNWVRQAPGKGLEWVG |
| NO: 4702 | (VH-CH)- | YIHRSGSTSYNPSLKSRFTISRDTSKNTFYLQMNSLRAEDTAVYYCARGDWH |
| | Hole | YFDYWGQGTMVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNHK |
| | | PSNTKVDKRVEPKSCDKTHTCPPCPAPELLGGPSVFLFPPKPKDTLMISRTPE |
| | | VTCVVVDVSHEDPEVKFNWYVDGVEVHNAKTKPREEQYASTYRVVSVLTV |
| | | LHQDWLNGKEYKCKVSNKALPAPIEKTISKAKGQPREPQVCTLPPSREEMTK |
| | | NQVSLSCAVKGFYPSDIAVEWESNGQPENNYKTTPPVLDSDGSFFLVSKLTV |
| | | DKSRWQQGNVFSCSVMHEALHNHYTQKSLSLSP |
| SEQ ID | Light chain | DSVTTQSPLSLPVTLGQPASISCSGEKLSDKYVHWYQQRPGQSPRMLIYEND |
| NO: 501 | (VL-CL) | RRPSGVPDRFSGSNSGNDATLKISRVEAEDVGVYFCQFWDSTNSAVFGGGT |
| | | KVEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPVT |
| | | KSFNRGEC |
| SEQ ID | IL- | ITCPPPMSVEHADIWVKSYSLYSRERYICNSGFKRKAGTSSLTECVLNKATN |
| NO: 3420 | 15Rαsushi- | VAHWTTPSLKCIRDPALVHQRPAPPSGGSGGGGSGGGSGGGGSGGGNWVN |
| | IL-15 Heavy | VISDLKKIEDLIQSMHIDATLYTESDVHPSCKVTAMKCFLLELQVISLESGDA |
| | Chain | SIHDTVENLIILANNSLSSNGNVTESGCKECEELEEKNIKEFLQSFVHIVQMFI |
| | Constant | NTSGGGGSGGGGSDKTHTCPPCPAPELLGGPSVFLFPPKPKDTLMISRTPEVT |
| | Domain (Fc | CVVVDVSHEDPEVKFNWYVDGVEVHNAKTKPREEQYASTYRVVSVLTVLH |
| | Region) | QDWLNGKEYKCKVSNKALPAPIEKTISKAKGQPREPQVYTLPPCREEMTKN |
| | -Knob | QVSLWCLVKGFYPSDIAVEWESNGQPENNYKTTPPVLDSDGSFFLYSKLTV |
| | | DKSRWQQGNVFSCSVMHEALHNHYTQKSLSLSPGK |
| SEQ ID | IL- | ITCPPPMSVEHADIWVKSYSLYSRERYICNSGFKRKAGTSSLTECVLNKATN |
| NO: 4703 | 15Rαsushi- | VAHWTTPSLKCIRDPALVHQRPAPPSGGSGGGGSGGGSGGGGSGGGNWVN |
| | IL-15 Heavy | VISDLKKIEDLIQSMHIDATLYTESDVHPSCKVTAMKCFLLELQVISLESGDA |
| | Chain | SIHDTVENLIILANNSLSSNGNVTESGCKECEELEEKNIKEFLQSFVHIVQMFI |
| | Constant | NTSGGGGSGGGGSDKTHTCPPCPAPELLGGPSVFLFPPKPKDTLMISRTPEVT |
| | Domain (Fc | CVVVDVSHEDPEVKFNWYVDGVEVHNAKTKPREEQYASTYRVVSVLTVLH |
| | Region) | QDWLNGKEYKCKVSNKALPAPIEKTISKAKGQPREPQVYTLPPCREEMTKN |
| | -Knob | QVSLWCLVKGFYPSDIAVEWESNGQPENNYKTTPPVLDSDGSFFLYSKLTV |
| | | DKSRWQQGNVFSCSVMHEALHNHYTQKSLSLSP |
| SEQ ID | VHCDR1 | GFSIIRVGYHWN |
| NO: 502 | | |
| SEQ ID | VHCDR2 | YIHRSGSTSYNPSLKS |
| NO: 503 | | |
| SEQ ID | VHCDR3 | GDWHYFDY |
| NO: 504 | | |
| SEQ ID | LC CDR1 | SGEKLSDKYVH |
| NO: 26 | | |
| SEQ ID | LC CDR2 | ENDRRPS |
| NO: 28 | | |
| SEQ ID | LC CDR3 | QFWDSTNSAV |
| NO: 86 | | |
| SEQ ID | VH (BM130) | EIQLLESGGGLVQPGGSLRLSCAVSGFSIIRVGYHWNWVRQAPGKGLEWVGY |
| NO: 505 | | IHRSGSTSYNPSLKSRFTISRDTSKNTFYLQMNSLRAEDTAVYYCARGDWHYF |
| | | DYWGQGTMVTVSS |
| SEQ ID | VL (BM130) | DSVTTQSPLSLPVTLGQPASISCSGEKLSDKYVHWYQQRPGQSPRMLIYENDR |
| NO: 13 | | RPSGVPDRFSGSNSGNDATLKISRVEAEDVGVYFCQFWDSTNSAVFGGGTKV |
| | | EIK |
| SEQ ID | Heavy chain | ASTKGPSVFPLAPSSKSTSGGTAALGCLVKDYFPEPVTVSWNSGALTSGVHTF |
| NO: 3649 | constant | PAVLQSSGLYSLSSVVTVPSSSLGTQTYICNVNHKPSNTKVDKRVEPKSCDKT |
| | region (CH1- | HTCPPCPAPELLGGPSVFLFPPKPKDTLMISRTPEVTCVVVDVSHEDPEVKFN |
| | CH2-CH3) | WYVDGVEVHNAKTKPREEQYASTYRVVSVLTVLHQDWLNGKEYKCKVSN |
| | Hole | KALPAPIEKTISKAKGQPREPQVCTLPPSREEMTKNQVSLSCAVKGFYPSDIAV |
| | | EWESNGQPENNYKTTPPVLDSDGSFFLVSKLTVDKSRWQQGNVFSCSVMHE |
| | | ALHNHYTQKSLSLSPGK |
| SEQ ID | Heavy chain | ASTKGPSVFPLAPSSKSTSGGTAALGCLVKDYFPEPVTVSWNSGALTSGVHTF |
| NO: 3451 | constant | PAVLQSSGLYSLSSVVTVPSSSLGTQTYICNVNHKPSNTKVDKRVEPKSCDKT |
| | region (CH1- | HTCPPCPAPELLGGPSVFLFPPKPKDTLMISRTPEVTCVVVDVSHEDPEVKFN |
| | CH2-CH3) | WYVDGVEVHNAKTKPREEQYASTYRVVSVLTVLHQDWLNGKEYKCKVSN |
| | Hole | KALPAPIEKTISKAKGQPREPQVCTLPPSREEMTKNQVSLSCAVKGFYPSDIAV |
| | | EWESNGQPENNYKTTPPVLDSDGSFFLVSKLTVDKSRWQQGNVFSCSVMHE |
| | | ALHNHYTQKSLSLSP |
| SEQ ID | Heavy chain | EPKSCDKTHTCPPCPAPELLGGPSVFLFPPKPKDTLMISRTPEVTCVVVDVSHE |
| NO: 506 | hinge-Fc | DPEVKFNWYVDGVEVHNAKTKPREEQYASTYRVVSVLTVLHQDWLNGKEY |
| | region (CH2- | KCKVSNKALPAPIEKTISKAKGQPREPQVCTLPPSREEMTKNQVSLSCAVKGF |
| | CH3) Hole | YPSDIAVEWESNGQPENNYKTTPPVLDSDGSFFLVSKLTVDKSRWQQGNVFS |
| | | CSVMHEALHNHYTQKSLSLSPGK |
| SEQ ID | Heavy chain | EPKSCDKTHTCPPCPAPELLGGPSVFLFPPKPKDTLMISRTPEVTCVVVDVSHE |
| NO: 507 | hinge-Fc | DPEVKFNWYVDGVEVHNAKTKPREEQYASTYRVVSVLTVLHQDWLNGKEY |
| | region (CH2- | KCKVSNKALPAPIEKTISKAKGQPREPQVCTLPPSREEMTKNQVSLSCAVKGF |
| | CH3) Hole | YPSDIAVEWESNGQPENNYKTTPPVLDSDGSFFLVSKLTVDKSRWQQGNVFS |
| | | CSVMHEALHNHYTQKSLSLSP |
| SEQ ID | Light chain | RTVAAPSVFIFPPSDEQLKSGTASVVCLLNNFYPREAKVQWKVDNALQSGNS |
| NO: 3644 | constant | QESVTEQDSKDSTYSLSSTLTLSKADYEKHKVYACEVTHQGLSSPVTKSFNR |
| | region | GEC |
| SEQ ID | IL-15Rαsushi | ITCPPPMSVEHADIWVKSYSLYSRERYICNSGFKRKAGTSSLTECVLNKATNV |
| NO: 3472 | (AA 31-108 | AHWTTPSLKCIRDPALVHQRPAPPS |
| | of IL- | |
| | 15Ralpha) | |
| SEQ ID | Linker | GGSGGGGSGGGSGGGGSGGG |
| NO: 3473 | | |
| SEQ ID | AA 49-162 of | NWVNVISDLKKIEDLIQSMHIDATLYTESDVHPSCKVTAMKCFLLELQVISLE |
| NO: 2170 | IL-15 | SGDASIHDTVENLIILANNSLSSNGNVTESGCKECEELEEKNIKEFLQSFVHIVQ |
| | | MFINTS |
| SEQ ID | IL- | ITCPPPMSVEHADIWVKSYSLYSRERYICNSGFKRKAGTSSLTECVLNKATNV |
| NO: 3474 | 15Rαsushi- | AHWTTPSLKCIRDPALVHQRPAPPSGGSGGGGSGGGSGGGGSGGGNWVNVI |
| | IL-15 (AA | SDLKKIEDLIQSMHIDATLYTESDVHPSCKVTAMKCFLLELQVISLESGDASIH |
| | 31-108 of IL- | DTVENLIILANNSLSSNGNVTESGCKECEELEEKNIKEFLQSFVHIVQMFINTS |
| | 15Ralpha- | |
| | Linker-AA | |
| | 31-108 of IL- | |
| | 15Ralpha) | |
| SEQ ID | Linker | GGGGSGGGGS |
| NO: 3308 | | |
| SEQ ID | Heavy chain | DKTHTCPPCPAPELLGGPSVFLFPPKPKDTLMISRTPEVTCVVVDVSHEDPEV |
| NO: 3648 | hinge-Fc | KFNWYVDGVEVHNAKTKPREEQYASTYRVVSVLTVLHQDWLNGKEYKCK |
| | region (CH2- | VSNKALPAPIEKTISKAKGQPREPQVYTLPPCREEMTKNQVSLWCLVKGFYPS |
| | CH3) knob | DIAVEWESNGQPENNYKTTPPVLDSDGSFFLYSKLTVDKSRWQQGNVFSCSV |
| | | MHEALHNHYTQKSLSLSPGK |
| SEQ ID | Heavy chain | DKTHTCPPCPAPELLGGPSVFLFPPKPKDTLMISRTPEVTCVVVDVSHEDPEV |
| NO: 508 | hinge-Fc | KFNWYVDGVEVHNAKTKPREEQYASTYRVVSVLTVLHQDWLNGKEYKCK |
| | region (CH2- | VSNKALPAPIEKTISKAKGQPREPQVYTLPPCREEMTKNQVSLWCLVKGFYPS |
| | CH3) knob | DIAVEWESNGQPENNYKTTPPVLDSDGSFFLYSKLTVDKSRWQQGNVFSCSV |
| | | MHEALHNHYTQKSLSLSP |

Exemplary molecule 2

| | | |
|---|---|---|
| SEQ ID | Heavy chain | EIQLLESGGGLVQPGGSLRLSCAVSGFSIIRVGYHWNWVRQAPGKGLEWVG |
| NO: 500 | (VH-CH)- | YIHRSGSTSYNPSLKSRFTISRDTSKNTFYLQMNSLRAEDTAVYYCARGDWH |
| | Hole | YFDYWGQGTMVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNHK |
| | | PSNTKVDKRVEPKSCDKTHTCPPCPAPELLGGPSVFLFPPKPKDTLMISRTPE |
| | | VTCVVVDVSHEDPEVKFNWYVDGVEVHNAKTKPREEQYASTYRVVSVLTV |
| | | LHQDWLNGKEYKCKVSNKALPAPIEKTISKAKGQPREPQVCTLPPSREEMTK |
| | | NQVSLSCAVKGFYPSDIAVEWESNGQPENNYKTTPPVLDSDGSFFLVSKLTV |
| | | DKSRWQQGNVFSCSVMHEALHNHYTQKSLSLSPGK |
| SEQ ID | Heavy chain | EIQLLESGGGLVQPGGSLRLSCAVSGFSIIRVGYHWNWVRQAPGKGLEWVG |
| NO: 4702 | (VH-CH)- | YIHRSGSTSYNPSLKSRFTISRDTSKNTFYLQMNSLRAEDTAVYYCARGDWH |
| | Hole | YFDYWGQGTMVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNHK |
| | | PSNTKVDKRVEPKSCDKTHTCPPCPAPELLGGPSVFLFPPKPKDTLMISRTPE |
| | | VTCVVVDVSHEDPEVKFNWYVDGVEVHNAKTKPREEQYASTYRVVSVLTV |
| | | LHQDWLNGKEYKCKVSNKALPAPIEKTISKAKGQPREPQVCTLPPSREEMTK |
| | | NQVSLSCAVKGFYPSDIAVEWESNGQPENNYKTTPPVLDSDGSFFLVSKLTV |
| | | DKSRWQQGNVFSCSVMHEALHNHYTQKSLSLSP |
| SEQ ID | Light chain | DSVTTQSPLSLPVTLGQPASISCSGEKLSDKYVHWYQQRPGQSPRMLIYEND |
| NO: 501 | (VL-CL) | RRPSGVPDRFSGSNSGNDATLKISRVEAEDVGVYFCQFWDSTNSAVFGGGT |
| | | KVEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPVT |
| | | KSFNRGEC |
| SEQ ID | IL- | ITCPPPMSVEHADIWVKSYSLYSRERYICNSGFKRKAGTSSLTECVLNKATN |
| NO: 3669 | 15Rαsushi- | VAHWTTPSLKCIRDPALVHQRPAPPSGGSGGGGSGGGSGGGGSGGGNWVN |
| | IL-15 Heavy | VISDLKKIEDLIQSMHIDATLYTESDVHPSCKVTAMKCFLLELQVISLESGDA |
| | Chain | SIHDTVENLIILANNSLSSNGNVTESGCKECEELEEKNIKEFLQSFVHIVAMFI |
| | Constant | NTSGGGGSGGGGSDKTHTCPPCPAPELLGGPSVFLFPPKPKDTLMISRTPEVT |
| | Domain (Fc | CVVVDVSHEDPEVKFNWYVDGVEVHNAKTKPREEQYASTYRVVSVLTVLH |
| | Region) | QDWLNGKEYKCKVSNKALPAPIEKTISKAKGQPREPQVYTLPPCREEMTKN |
| | -Knob | QVSLWCLVKGFYPSDIAVEWESNGQPENNYKTTPPVLDSDGSFFLYSKLTV |
| | | DKSRWQQGNVFSCSVMHEALHNHYTQKSLSLSPGK |
| SEQ ID | IL- | ITCPPPMSVEHADIWVKSYSLYSRERYICNSGFKRKAGTSSLTECVLNKATN |
| NO: 4708 | 15Rαsushi- | VAHWTTPSLKCIRDPALVHQRPAPPSGGSGGGGSGGGSGGGGSGGGNWVN |
| | IL-15 Heavy | VISDLKKIEDLIQSMHIDATLYTESDVHPSCKVTAMKCFLLELQVISLESGDA |
| | Chain | SIHDTVENLIILANNSLSSNGNVTESGCKECEELEEKNIKEFLQSFVHIVAMFI |
| | Constant | NTSGGGGSGGGGSDKTHTCPPCPAPELLGGPSVFLFPPKPKDTLMISRTPEVT |
| | Domain (Fc | CVVVDVSHEDPEVKFNWYVDGVEVHNAKTKPREEQYASTYRVVSVLTVLH |
| | Region) | QDWLNGKEYKCKVSNKALPAPIEKTISKAKGQPREPQVYTLPPCREEMTKN |
| | -Knob | QVSLWCLVKGFYPSDIAVEWESNGQPENNYKTTPPVLDSDGSFFLYSKLTV |
| | | DKSRWQQGNVFSCSVMHEALHNHYTQKSLSLSP |
| SEQ ID | VHCDR1 | GFSIIRVGYHWN |
| NO: 502 | | |
| SEQ ID | VHCDR2 | YIHRSGSTSYNPSLKS |
| NO: 503 | | |
| SEQ ID | VHCDR3 | GDWHYFDY |
| NO: 504 | | |
| SEQ ID | LC CDR1 | SGEKLSDKYVH |
| NO: 26 | | |
| SEQ ID | LC CDR2 | ENDRRPS |
| NO: 28 | | |
| SEQ ID | LC CDR3 | QFWDSTNSAV |
| NO: 86 | | |
| SEQ ID | VH (BM130) | EIQLLESGGGLVQPGGSLRLSCAVSGFSIIRVGYHWNWVRQAPGKGLEWVGY |
| NO: 505 | | IHRSGSTSYNPSLKSRFTISRDTSKNTFYLQMNSLRAEDTAVYYCARGDWHYF |
| | | DYWGQGTMVTVSS |
| SEQ ID | VL (BM130) | DSVTTQSPLSLPVTLGQPASISCSGEKLSDKYVHWYQQRPGQSPRMLIYENDR |
| NO: 13 | | RPSGVPDRFSGSNSGNDATLKISRVEAEDVGVYFCQFWDSTNSAVFGGGTKV |
| | | EIK |
| SEQ ID | Heavy chain | ASTKGPSVFPLAPSSKSTSGGTAALGCLVKDYFPEPVTVSWNSGALTSGVHTF |
| NO: 3649 | constant | PAVLQSSGLYSLSSVVTVPSSSLGTQTYICNVNHKPSNTKVDKRVEPKSCDKT |
| | region (CH1- | HTCPPCPAPELLGGPSVFLFPPKPKDTLMISRTPEVTCVVVDVSHEDPEVKFN |
| | CH2-CH3) | WYVDGVEVHNAKTKPREEQYASTYRVVSVLTVLHQDWLNGKEYKCKVSN |
| | Hole | KALPAPIEKTISKAKGQPREPQVCTLPPSREEMTKNQVSLSCAVKGFYPSDIAV |
| | | EWESNGQPENNYKTTPPVLDSDGSFFLVSKLTVDKSRWQQGNVFSCSVMHE |
| | | ALHNHYTQKSLSLSPGK |
| SEQ ID | Heavy chain | ASTKGPSVFPLAPSSKSTSGGTAALGCLVKDYFPEPVTVSWNSGALTSGVHTF |
| NO: 3451 | constant | PAVLQSSGLYSLSSVVTVPSSSLGTQTYICNVNHKPSNTKVDKRVEPKSCDKT |
| | region (CH1- | HTCPPCPAPELLGGPSVFLFPPKPKDTLMISRTPEVTCVVVDVSHEDPEVKFN |
| | CH2-CH3) | WYVDGVEVHNAKTKPREEQYASTYRVVSVLTVLHQDWLNGKEYKCKVSN |
| | Hole | KALPAPIEKTISKAKGQPREPQVCTLPPSREEMTKNQVSLSCAVKGFYPSDIAV |
| | | EWESNGQPENNYKTTPPVLDSDGSFFLVSKLTVDKSRWQQGNVFSCSVMHE |
| | | ALHNHYTQKSLSLSP |
| SEQ ID | Heavy chain | EPKSCDKTHTCPPCPAPELLGGPSVFLFPPKPKDTLMISRTPEVTCVVVDVSHE |
| NO: 506 | hinge-Fc | DPEVKFNWYVDGVEVHNAKTKPREEQYASTYRVVSVLTVLHQDWLNGKEY |
| | region (CH2- | KCKVSNKALPAPIEKTISKAKGQPREPQVCTLPPSREEMTKNQVSLSCAVKGF |
| | CH3) Hole | YPSDIAVEWESNGQPENNYKTTPPVLDSDGSFFLVSKLTVDKSRWQQGNVFS |
| | | CSVMHEALHNHYTQKSLSLSPGK |
| SEQ ID | Heavy chain | EPKSCDKTHTCPPCPAPELLGGPSVFLFPPKPKDTLMISRTPEVTCVVVDVSHE |
| NO: 507 | hinge-Fc | DPEVKFNWYVDGVEVHNAKTKPREEQYASTYRVVSVLTVLHQDWLNGKEY |
| | region (CH2- | KCKVSNKALPAPIEKTISKAKGQPREPQVCTLPPSREEMTKNQVSLSCAVKGF |
| | CH3) Hole | YPSDIAVEWESNGQPENNYKTTPPVLDSDGSFFLVSKLTVDKSRWQQGNVFS |
| | | CSVMHEALHNHYTQKSLSLSP |
| SEQ ID | Light chain | RTVAAPSVFIFPPSDEQLKSGTASVVCLLNNFYPREAKVQWKVDNALQSGNS |
| NO: 3644 | constant | QESVTEQDSKDSTYSLSSTLTLSKADYEKHKVYACEVTHQGLSSPVTKSFNR |
| | region | GEC |
| SEQ ID | IL-15Rαsushi | ITCPPPMSVEHADIWVKSYSLYSRERYICNSGFKRKAGTSSLTECVLNKATNV |
| NO: 3472 | (AA 31-108 | AHWTTPSLKCIRDPALVHQRPAPPS |
| | of IL- | |
| | 15Ralpha) | |
| SEQ ID | Linker | GGSGGGGSGGGSGGGGSGGG |
| NO: 3473 | | |
| SEQ ID | AA 49-162 of | NWVNVISDLKKIEDLIQSMHIDATLYTESDVHPSCKVTAMKCFLLELQVISLE |
| NO: 3670 | IL-15 | SGDASIHDTVENLIILANNSLSSNGNVTESGCKECEELEEKNIKEFLQSFVH |
| | | IVAMFINTS |
| SEQ ID | IL- | ITCPPPMSVEHADIWVKSYSLYSRERYICNSGFKRKAGTSSLTECVLNKATNV |
| NO: 3671 | 15Rαsushi- | AHWTTPSLKCIRDPALVHQRPAPPSGGSGGGGSGGGSGGGGSGGGNWVNVI |
| | IL-15 (AA | SDLKKIEDLIQSMHIDATLYTESDVHPSCKVTAMKCFLLELQVISLESGDASIH |
| | 31-108 of IL- | DTVENLIILANNSLSSNGNVTESGCKECEELEEKNIKEFLQSFVHIVAMFINTS |
| | 15Ralpha- | |
| | Linker-AA | |
| | 31-108 of IL- | |
| | 15Ralpha) | |
| SEQ ID | Linker | GGGGSGGGGS |
| NO: 3308 | | |
| SEQ ID | Heavy chain | DKTHTCPPCPAPELLGGPSVFLFPPKPKDTLMISRTPEVTCVVVDVSHEDPEV |
| NO: 3648 | hinge-Fc | KFNWYVDGVEVHNAKTKPREEQYASTYRVVSVLTVLHQDWLNGKEYKCK |
| | region (CH2- | VSNKALPAPIEKTISKAKGQPREPQVYTLPPCREEMTKNQVSLWCLVKGFYPS |
| | CH3) knob | DIAVEWESNGQPENNYKTTPPVLDSDGSFFLYSKLTVDKSRWQQGNVFSCSV |
| | | MHEALHNHYTQKSLSLSPGK |
| SEQ ID | Heavy chain | KTHTCPPCPAPELLGGPSVFLFPPKPKDTLMISRTPEVTCVVVDVSHEDPEVKF |
| NO: 8686 | hinge-Fc | NWYVDGVEVHNAKTKPREEQYASTYRVVSVLTVLHQDWLNGKEYKCKVS |
| | region (CH2- | NKALPAPIEKTISKAKGQPREPQVYTLPPCREEMTKNQVSLWCLVKGFYPSDI |
| | CH3) knob | AVEWESNGQPENNYKTTPPVLDSDGSFFLYSKLTVDKSRWQQGNVFSCSVM |
| | | HEALHNHYTQKSLSLSP |

Exemplary molecule 3

| | | |
|---|---|---|
| SEQ ID | Heavy chain | EIQLLESGGGLVQPGGSLRLSCAVSGFSIIRVGYHWNWVRQAPGKGLEWVG |
| NO: 500 | (VH-CH)- | YIHRSGSTSYNPSLKSRFTISRDTSKNTFYLQMNSLRAEDTAVYYCARGDWH |
| | Hole | YFDYWGQGTMVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNHK |
| | | PSNTKVDKRVEPKSCDKTHTCPPCPAPELLGGPSVFLFPPKPKDTLMISRTPE |
| | | VTCVVVDVSHEDPEVKFNWYVDGVEVHNAKTKPREEQYASTYRVVSVLTV |
| | | LHQDWLNGKEYKCKVSNKALPAPIEKTISKAKGQPREPQVCTLPPSREEMTK |
| | | NQVSLSCAVKGFYPSDIAVEWESNGQPENNYKTTPPVLDSDGSFFLVSKLTV |
| | | DKSRWQQGNVFSCSVMHEALHNHYTQKSLSLSPGK |
| SEQ ID | Heavy chain | EIQLLESGGGLVQPGGSLRLSCAVSGFSIIRVGYHWNWVRQAPGKGLEWVG |
| NO: 4702 | (VH-CH)- | YIHRSGSTSYNPSLKSRFTISRDTSKNTFYLQMNSLRAEDTAVYYCARGDWH |
| | Hole | YFDYWGQGTMVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNHK |
| | | PSNTKVDKRVEPKSCDKTHTCPPCPAPELLGGPSVFLFPPKPKDTLMISRTPE |
| | | VTCVVVDVSHEDPEVKFNWYVDGVEVHNAKTKPREEQYASTYRVVSVLTV |
| | | LHQDWLNGKEYKCKVSNKALPAPIEKTISKAKGQPREPQVCTLPPSREEMTK |
| | | NQVSLSCAVKGFYPSDIAVEWESNGQPENNYKTTPPVLDSDGSFFLVSKLTV |
| | | DKSRWQQGNVFSCSVMHEALHNHYTQKSLSLSP |
| SEQ ID | Light chain | DSVTTQSPLSLPVTLGQPASISCSGEKLSDKYVHWYQQRPGQSPRMLIYEND |
| NO: 501 | (VL-CL) | RRPSGVPDRFSGSNSGNDATLKISRVEAEDVGVYFCQFWDSTNSAVFGGGT |
| | | KVEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPVT |
| | | KSFNRGEC |
| SEQ ID | IL-2 C125A- | APTSSSTKKTQLQLEHLLLDLQMILNGINNYKNPKLTRMLTFKFYMPKKATE |
| NO: 3340 | Heavy Chain | LKHLQCLEEELKPLEEVLNLAQSKNFHLRPRDLISNINVIVLELKGSETTFMC |
| | Constant | EYADETATIVEFLNRWITFAQSIISTLTGGGGSGGGGSDKTHTCPPCPAPELL |
| | Domain (Fc | GGPSVFLFPPKPKDTLMISRTPEVTCVVVDVSHEDPEVKFNWYVDGVEVHN |
| | Region) | AKTKPREEQYASTYRVVSVLTVLHQDWLNGKEYKCKVSNKALPAPIEKTIS |
| | | KAKGQPREPQVYTLPPCREEMTKNQVSLWCLVKGFYPSDIAVEWESNGQPE |
| | | NNYKTTPPVLDSDGSFFLYSKLTVDKSRWQQGNVFSCSVMHEALHNHYTQ |
| | | KSLSLSPGK |
| SEQ ID | IL-2 C125A- | APTSSSTKKTQLQLEHLLLDLQMILNGINNYKNPKLTRMLTFKFYMPKKATE |
| NO: 4706 | Heavy Chain | LKHLQCLEEELKPLEEVLNLAQSKNFHLRPRDLISNINVIVLELKGSETTFMC |
| | Constant | EYADETATIVEFLNRWITFAQSIISTLTGGGGSGGGGSDKTHTCPPCPAPELL |
| | Domain (Fc | GGPSVFLFPPKPKDTLMISRTPEVTCVVVDVSHEDPEVKFNWYVDGVEVHN |
| | Region) | AKTKPREEQYASTYRVVSVLTVLHQDWLNGKEYKCKVSNKALPAPIEKTIS |
| | | KAKGQPREPQVYTLPPCREEMTKNQVSLWCLVKGFYPSDIAVEWESNGQPE |
| | | NNYKTTPPVLDSDGSFFLYSKLTVDKSRWQQGNVFSCSVMHEALHNHYTQ |
| | | KSLSLSP |
| SEQ ID | VHCDR1 | GFSIIRVGYHWN |
| NO: 502 | | |
| SEQ ID | VHCDR2 | YIHRSGSTSYNPSLKS |
| NO: 503 | | |
| SEQ ID | VHCDR3 | GDWHYFDY |
| NO: 504 | | |
| SEQ ID | LC CDR1 | SGEKLSDKYVH |
| NO: 26 | | |
| SEQ ID | LC CDR2 | ENDRRPS |
| NO: 28 | | |
| SEQ ID | LC CDR3 | QFWDSTNSAV |
| NO: 86 | | |
| SEQ ID | VH (BM130) | EIQLLESGGGLVQPGGSLRLSCAVSGFSIIRVGYHWNWVRQAPGKGLEWVGY |
| NO: 505 | | IHRSGSTSYNPSLKSRFTISRDTSKNTFYLQMNSLRAEDTAVYYCARGDWHYF |
| | | DYWGQGTMVTVSS |
| SEQ ID | VL (BM130) | DSVTTQSPLSLPVTLGQPASISCSGEKLSDKYVHWYQQRPGQSPRMLIYENDR |
| NO: 13 | | RPSGVPDRFSGSNSGNDATLKISRVEAEDVGVYFCQFWDSTNSAVFGGGTKV |
| | | EIK |
| SEQ ID | Heavy chain | ASTKGPSVFPLAPSSKSTSGGTAALGCLVKDYFPEPVTVSWNSGALTSGVHTF |
| NO: 3649 | constant | PAVLQSSGLYSLSSVVTVPSSSLGTQTYICNVNHKPSNTKVDKRVEPKSCDKT |
| | region (CH1- | HTCPPCPAPELLGGPSVFLFPPKPKDTLMISRTPEVTCVVVDVSHEDPEVKFN |
| | CH2-CH3) | WYVDGVEVHNAKTKPREEQYASTYRVVSVLTVLHQDWLNGKEYKCKVSN |
| | Hole | KALPAPIEKTISKAKGQPREPQVCTLPPSREEMTKNQVSLSCAVKGFYPSDIAV |
| | | EWESNGQPENNYKTTPPVLDSDGSFFLVSKLTVDKSRWQQGNVFSCSVMHE |
| | | ALHNHYTQKSLSLSPGK |
| SEQ ID | Heavy chain | ASTKGPSVFPLAPSSKSTSGGTAALGCLVKDYFPEPVTVSWNSGALTSGVHTF |
| NO: 3451 | constant | PAVLQSSGLYSLSSVVTVPSSSLGTQTYICNVNHKPSNTKVDKRVEPKSCDKT |
| | region (CH1- | HTCPPCPAPELLGGPSVFLFPPKPKDTLMISRTPEVTCVVVDVSHEDPEVKFN |
| | CH2-CH3) | WYVDGVEVHNAKTKPREEQYASTYRVVSVLTVLHQDWLNGKEYKCKVSN |
| | Hole | KALPAPIEKTISKAKGQPREPQVCTLPPSREEMTKNQVSLSCAVKGFYPSDIAV |
| | | EWESNGQPENNYKTTPPVLDSDGSFFLVSKLTVDKSRWQQGNVFSCSVMHE |
| | | ALHNHYTQKSLSLSP |
| SEQ ID | Heavy chain | EPKSCDKTHTCPPCPAPELLGGPSVFLFPPKPKDTLMISRTPEVTCVVVDVSHE |
| NO: 506 | hinge-Fc | DPEVKFNWYVDGVEVHNAKTKPREEQYASTYRVVSVLTVLHQDWLNGKEY |
| | region (CH2- | KCKVSNKALPAPIEKTISKAKGQPREPQVCTLPPSREEMTKNQVSLSCAVKGF |
| | CH3) Hole | YPSDIAVEWESNGQPENNYKTTPPVLDSDGSFFLVSKLTVDKSRWQQGNVFS |
| | | CSVMHEALHNHYTQKSLSLSPGK |
| SEQ ID | Heavy chain | EPKSCDKTHTCPPCPAPELLGGPSVFLFPPKPKDTLMISRTPEVTCVVVDVSHE |
| NO: 507 | hinge-Fc | DPEVKFNWYVDGVEVHNAKTKPREEQYASTYRVVSVLTVLHQDWLNGKEY |
| | region (CH2- | KCKVSNKALPAPIEKTISKAKGQPREPQVCTLPPSREEMTKNQVSLSCAVKGF |
| | CH3) Hole | YPSDIAVEWESNGQPENNYKTTPPVLDSDGSFFLVSKLTVDKSRWQQGNVFS |
| | | CSVMHEALHNHYTQKSLSLSP |
| SEQ ID | Light chain | RTVAAPSVFIFPPSDEQLKSGTASVVCLLNNFYPREAKVQWKVDNALQSGNS |
| NO: 3644 | constant | QESVTEQDSKDSTYSLSSTLTLSKADYEKHKVYACEVTHQGLSSPVTKSFNR |
| | region | GEC |
| SEQ ID | IL-2 C125A | APTSSSTKKTQLQLEHLLLDLQMILNGINNYKNPKLTRMLTFKFYMPKKATE |
| NO: 2270 | | LKHLQCLEEELKPLEEVLNLAQSKNFHLRPRDLISNINVIVLELKGSETTFMCE |
| | | YADETATIVEFLNRWITFAQSIISTLT |
| SEQ ID | Linker | GGGGSGGGGS |
| NO: 3308 | | |
| SEQ ID | Heavy chain | DKTHTCPPCPAPELLGGPSVFLFPPKPKDTLMISRTPEVTCVVVDVSHEDPEV |
| NO: 3648 | hinge-Fc | KFNWYVDGVEVHNAKTKPREEQYASTYRVVSVLTVLHQDWLNGKEYKCK |
| | region (CH2- | VSNKALPAPIEKTISKAKGQPREPQVYTLPPCREEMTKNQVSLWCLVKGFYPS |
| | CH3) knob | DIAVEWESNGQPENNYKTTPPVLDSDGSFFLYSKLTVDKSRWQQGNVFSCSV |
| | | MHEALHNHYTQKSLSLSPGK |
| SEQ ID | Heavy chain | DKTHTCPPCPAPELLGGPSVFLFPPKPKDTLMISRTPEVTCVVVDVSHEDPEV |
| NO: 508 | hinge-Fc | KFNWYVDGVEVHNAKTKPREEQYASTYRVVSVLTVLHQDWLNGKEYKCK |
| | region (CH2- | VSNKALPAPIEKTISKAKGQPREPQVYTLPPCREEMTKNQVSLWCLVKGFYPS |
| | CH3) knob | DIAVEWESNGQPENNYKTTPPVLDSDGSFFLYSKLTVDKSRWQQGNVFSCSV |
| | | MHEALHNHYTQKSLSLSP |

Exemplary molecule 4 Anti-TRBC1-NKp30

| | | |
|---|---|---|
| SEQ ID | NKp30 scFv- | EIQLLESGGGLVQPGGSLRLSCAVSGFSIIRVGYHWNWVRQAPGKGLEWVGY |
| NO: 509 | CH-Hole | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | ATLKISRVEAEDVGVYFCQFWDSTNSAVFGGGTKVEIKDKTHTCPPCPAPELL |
| | | GGPSVFLFPPKPKDTLMISRTPEVTCVVVDVSHEDPEVKFNWYVDGVEVHNA |
| | | KTKPREEQYASTYRVVSVLTVLHQDWLNGKEYKCKVSNKALPAPIEKTISKA |
| | | KGQPREPQVCTLPPSREEMTKNQVSLSCAVKGFYPSDIAVEWESNGQPENNY |
| | | KTTPPVLDSDGSFFLVSKLTVDKSRWQQGNVFSCSVMHEALHNHYTQKSLSL |
| | | SPGK |
| SEQ ID | NKp30 scFv- | EIQLLESGGGLVQPGGSLRLSCAVSGFSIIRVGYHWNWVRQAPGKGLEWVGY |
| NO: 4704 | CH-Hole | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | ATLKISRVEAEDVGVYFCQFWDSTNSAVFGGGTKVEIKDKTHTCPPCPAPELL |
| | | GGPSVFLFPPKPKDTLMISRTPEVTCVVVDVSHEDPEVKFNWYVDGVEVHNA |
| | | KTKPREEQYASTYRVVSVLTVLHQDWLNGKEYKCKVSNKALPAPIEKTISKA |
| | | KGQPREPQVCTLPPSREEMTKNQVSLSCAVKGFYPSDIAVEWESNGQPENNY |
| | | KTTPPVLDSDGSFFLVSKLTVDKSRWQQGNVFSCSVMHEALHNHYTQKSLSL |
| | | SP |
| SEQ ID | Light chain | DVVMTQSPLSLPVTLGQPASISCRSSQRLVHSNGNTYLHWYQQRPGQSPRLLI |
| NO: 7380 | (VL-CL) | YRVSNRFPGVPDRFSGSGSGTDFTLKISRVEAEDVGVYFCSQSTHVPYTFGGG |
| | | TKVEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSPVT |
| | | KSFNRGEC |
| SEQ ID | Heavy chain | QVQLVQSGAEVKKPGSSVKVSCKASGYTFTGYVMHWVRQAPGQGLEWMG |
| NO: 7382 | (TRBC1 VH- | FIIPIFGTANYAQKFQGRVTITSDKSTTTAYMELSSLRSEDTAVYYCARGAGY |
| | CH)-knob | NFDGAYRFFDFWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYASTYRVVS |
| | | VLTVLHQDWLNGKEYKCKVSNKALPAPIEKTISKAKGQPREPQVYTLPPCRE |
| | | EMTKNQVSLWCLVKGFYPSDIAVEWESNGQPENNYKTTPPVLDSDGSFFLYS |
| | | KLTVDKSRWQQGNVFSCSVMHEALHNHYTQKSLSLSPGK |
| SEQ ID | Heavy chain | QVQLVQSGAEVKKPGSSVKVSCKASGYTFTGYVMHWVRQAPGQGLEWMG |
| NO: 4677 | (TRBC1 VH- | FIIPIFGTANYAQKFQGRVTITSDKSTTTAYMELSSLRSEDTAVYYCARGAGY |
| | CH)-knob | NFDGAYRFFDFWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYASTYRVVS |
| | | VLTVLHQDWLNGKEYKCKVSNKALPAPIEKTISKAKGQPREPQVYTLPPCRE |
| | | EMTKNQVSLWCLVKGFYPSDIAVEWESNGQPENNYKTTPPVLDSDGSFFLYS |
| | | KLTVDKSRWQQGNVFSCSVMHEALHNHYTQKSLSLSP |
| SEQ ID | NKp30 | GFSIIRVGYHWN |
| NO: 502 | VHCDR1 | |
| SEQ ID | NKp30 | YIHRSGSTSYNPSLKS |
| NO: 503 | VHCDR2 | |
| SEQ ID | NKp30 | GDWHYFDY |
| NO: 504 | VHCDR3 | |
| SEQ ID | NKp30 | SGEKLSDKYVH |
| NO: 26 | VLCDR1 | |
| SEQ ID | NKp30 | ENDRRPS |
| NO: 28 | VLCDR2 | |
| SEQ ID | NKp30 | QFWDSTNSAV |
| NO: 86 | VLCDR3 | |
| SEQ ID | NKp30 VH | EIQLLESGGGLVQPGGSLRLSCAVSGFSIIRVGYHWNWVRQAPGKGLEWVGY |
| NO: 505 | (BM130) | IHRSGSTSYNPSLKSRFTISRDTSKNTFYLQMNSLRAEDTAVYYCARGDWHYF |
| | | DYWGQGTMVTVSS |
| SEQ ID | NKp30 VL | DSVTTQSPLSLPVTLGQPASISCSGEKLSDKYVHWYQQRPGQSPRMLIYENDR |
| NO: 13 | (BM130) | RPSGVPDRFSGSNSGNDATLKISRVEAEDVGVYFCQFWDSTNSAVFGGGTKV |
| | | EIK |
| SEQ ID | Linker | GGGGSGGGGSGGGGSGGGGS |
| NO: 8291 | | |
| SEQ ID | Nkp30 scFv | EIQLLESGGGLVQPGGSLRLSCAVSGFSIIRVGYHWNWVRQAPGKGLEWVGY |
| NO: 518 | | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | ATLKISRVEAEDVGVYFCQFWDSTNSAVFGGGTKVEIK |
| SEQ ID | Heavy chain | DKTHTCPPCPAPELLGGPSVFLFPPKPKDTLMISRTPEVTCVVVDVSHEDPEV |
| NO: 3447 | hinge-Fc | KFNWYVDGVEVHNAKTKPREEQYASTYRVVSVLTVLHQDWLNGKEYKCK |
| | region (CH2- | VSNKALPAPIEKTISKAKGQPREPQVCTLPPSREEMTKNQVSLSCAVKGFYPS |
| | CH3) Hole | DIAVEWESNGQPENNYKTTPPVLDSDGSFFLVSKLTVDKSRWQQGNVFSCSV |
| | | MHEALHNHYTQKSLSLSPGK |
| SEQ ID | Heavy chain | DKTHTCPPCPAPELLGGPSVFLFPPKPKDTLMISRTPEVTCVVVDVSHEDPEV |
| NO: 511 | hinge-Fc | KFNWYVDGVEVHNAKTKPREEQYASTYRVVSVLTVLHQDWLNGKEYKCK |
| | region (CH2- | VSNKALPAPIEKTISKAKGQPREPQVCTLPPSREEMTKNQVSLSCAVKGFYPS |
| | CH3) Hole | DIAVEWESNGQPENNYKTTPPVLDSDGSFFLVSKLTVDKSRWQQGNVFSCSV |
| | | MHEALHNHYTQKSLSLSP |
| SEQ ID | TRBC 1 | GYVMH |
| NO: | VHCDR1 | |
| 8643 | | |
| SEQ ID | TRBC 1 | FIIPIFGTANYAQKFQG |
| NO: 7355 | VHCDR2 | |
| SEQ ID | TRBC 1 | GAGYNFDGAYRFFDF |
| NO: 202 | VHCDR3 | |
| SEQ ID | TRBC 1 | RSSQRLVHSNGNTYLH |
| NO: 223 | VLCDR1 | |
| SEQ ID | TRBC 1 | RVSNRFP |
| NO: 224 | VLCDR2 | |
| SEQ ID | TRBC 1 | SQSTHVPYT |
| NO: 225 | VLCDR3 | |
| SEQ ID | TRBC1 VH | QVQLVQSGAEVKKPGSSVKVSCKASGYTFTGYVMHWVRQAPGQGLEWMG |
| NO: 517 | | FIIPIFGTANYAQKFQGRVTITSDKSTTTAYMELSSLRSEDTAVYYCARGAGY |
| | | NFDGAYRFFDFWGQGTLVTVSS |
| SEQ ID | TRBC1 VL | DVVMTQSPLSLPVTLGQPASISCRSSQRLVHSNGNTYLHWYQQRPGQSPRLLI |
| NO: 8308 | | YRVSNRFPGVPDRFSGSGSGTDFTLKISRVEAEDVGVYFCSQSTHVPYTFGGG |
| | | TKVEIK |
| SEQ ID | Light chain | RTVAAPSVFIFPPSDEQLKSGTASVVCLLNNFYPREAKVQWKVDNALQSGNS |
| NO: 3644 | constant | QESVTEQDSKDSTYSLSSTLTLSKADYEKHKVYACEVTHQGLSSPVTKSFNR |
| | region | GEC |
| SEQ ID | Heavy chain | ASTKGPSVFPLAPSSKSTSGGTAALGCLVKDYFPEPVTVSWNSGALTSGVHTF |
| NO: 513 | constant | PAVLQSSGLYSLSSVVTVPSSSLGTQTYICNVNHKPSNTKVDKRVEPKSCDKT |
| | region (CH1- | HTCPPCPAPELLGGPSVFLFPPKPKDTLMISRTPEVTCVVVDVSHEDPEVKFN |
| | CH2-CH3) | WYVDGVEVHNAKTKPREEQYASTYRVVSVLTVLHQDWLNGKEYKCKVSN |
| | Knob | KALPAPIEKTISKAKGQPREPQVYTLPPCREEMTKNQVSLWCLVKGFYPSDIA |
| | | VEWESNGQPENNYKTTPPVLDSDGSFFLYSKLTVDKSRWQQGNVFSCSVMH |
| | | EALHNHYTQKSLSLSPGK |
| SEQ ID | Heavy chain | ASTKGPSVFPLAPSSKSTSGGTAALGCLVKDYFPEPVTVSWNSGALTSGVHTF |
| NO: 514 | constant | PAVLQSSGLYSLSSVVTVPSSSLGTQTYICNVNHKPSNTKVDKRVEPKSCDKT |
| | region (CH1- | HTCPPCPAPELLGGPSVFLFPPKPKDTLMISRTPEVTCVVVDVSHEDPEVKFN |
| | CH2-CH3) | WYVDGVEVHNAKTKPREEQYASTYRVVSVLTVLHQDWLNGKEYKCKVSN |
| | Knob | KALPAPIEKTISKAKGQPREPQVYTLPPCREEMTKNQVSLWCLVKGFYPSDIA |
| | | VEWESNGQPENNYKTTPPVLDSDGSFFLYSKLTVDKSRWQQGNVFSCSVMH |
| | | EALHNHYTQKSLSLSP |
| SEQ ID | Heavy chain | EPKSCDKTHTCPPCPAPELLGGPSVFLFPPKPKDTLMISRTPEVTCVVVDVSHE |
| NO: 515 | hinge-Fc | DPEVKFNWYVDGVEVHNAKTKPREEQYASTYRVVSVLTVLHQDWLNGKEY |
| | region (CH2- | KCKVSNKALPAPIEKTISKAKGQPREPQVYTLPPCREEMTKNQVSLWCLVKG |
| | CH3) Knob | FYPSDIAVEWESNGQPENNYKTTPPVLDSDGSFFLYSKLTVDKSRWQQGNVF |
| | | SCSVMHEALHNHYTQKSLSLSPGK |
| SEQ ID | Heavy chain | EPKSCDKTHTCPPCPAPELLGGPSVFLFPPKPKDTLMISRTPEVTCVVVDVSHE |
| NO: 516 | hinge-Fc | DPEVKFNWYVDGVEVHNAKTKPREEQYASTYRVVSVLTVLHQDWLNGKEY |
| | region (CH2- | KCKVSNKALPAPIEKTISKAKGQPREPQVYTLPPCREEMTKNQVSLWCLVKG |
| | CH3) Knob | FYPSDIAVEWESNGQPENNYKTTPPVLDSDGSFFLYSKLTVDKSRWQQGNVF |
| | | SCSVMHEALHNHYTQKSLSLSP |

Exemplary molecule 5 Anti-TRBC1-NKp30

| | | |
|---|---|---|
| SEQ ID | NKp30 scFv- | EIQLLESGGGLVQPGGSLRLSCAVSGFSIIRVGYHWNWVRQAPGKGLEWVGY |
| NO: 509 | CH-Hole | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | ATLKISRVEAEDVGVYFCQFWDSTNSAVFGGGTKVEIKDKTHTCPPCPAPELL |
| | | GGPSVFLFPPKPKDTLMISRTPEVTCVVVDVSHEDPEVKFNWYVDGVEVHNA |
| | | KTKPREEQYASTYRVVSVLTVLHQDWLNGKEYKCKVSNKALPAPIEKTISKA |
| | | KGQPREPQVCTLPPSREEMTKNQVSLSCAVKGFYPSDIAVEWESNGQPENNY |
| | | KTTPPVLDSDGSFFLVSKLTVDKSRWQQGNVFSCSVMHEALHNHYTQKSLSL |
| | | SPGK |
| SEQ ID | NKp30 scFv- | EIQLLESGGGLVQPGGSLRLSCAVSGFSIIRVGYHWNWVRQAPGKGLEWVGY |
| NO: 4704 | CH-Hole | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | ATLKISRVEAEDVGVYFCQFWDSTNSAVFGGGTKVEIKDKTHTCPPCPAPELL |
| | | GGPSVFLFPPKPKDTLMISRTPEVTCVVVDVSHEDPEVKFNWYVDGVEVHNA |
| | | KTKPREEQYASTYRVVSVLTVLHQDWLNGKEYKCKVSNKALPAPIEKTISKA |
| | | KGQPREPQVCTLPPSREEMTKNQVSLSCAVKGFYPSDIAVEWESNGQPENNY |
| | | KTTPPVLDSDGSFFLVSKLTVDKSRWQQGNVFSCSVMHEALHNHYTQKSLSL |
| | | SP |
| SEQ ID | Light chain | DVVMTQSPLSLPVTLGQPASISCRSSQRLVHSNGNTYLHWYQQRPGQSPRLLI |
| NO: 7380 | (VL-CL) | YRVSNRFPGVPDRFSGSGSGTDFTLKISRVEAEDVGVYFCSQSTHVPYTFGGG |
| | | TKVEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSPVT |
| | | KSFNRGEC |
| SEQ ID | Heavy chain | QVQLVQSGAEVKKPGSSVKVSCKASGYTFTGYVMHWVRQAPGQGLEWMG |
| NO: 7379 | (TRBC1 VH- | FINPYNDDIQSNERFRGRVTITSDKSTTTAYMELSSLRSEDTAVYYCARGAGY |
| | CH)-knob | NFDGAYRFFDFWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYASTYRVVS |
| | | VLTVLHQDWLNGKEYKCKVSNKALPAPIEKTISKAKGQPREPQVYTLPPCRE |
| | | EMTKNQVSLWCLVKGFYPSDIAVEWESNGQPENNYKTTPPVLDSDGSFFLYS |
| | | KLTVDKSRWQQGNVFSCSVMHEALHNHYTQKSLSLSPGK |
| SEQ ID | Heavy chain | QVQLVQSGAEVKKPGSSVKVSCKASGYTFTGYVMHWVRQAPGQGLEWMG |
| NO: 4675 | (TRBC1 VH- | FINPYNDDIQSNERFRGRVTITSDKSTTTAYMELSSLRSEDTAVYYCARGAGY |
| | CH)-knob | NFDGAYRFFDFWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYASTYRVVS |
| | | VLTVLHQDWLNGKEYKCKVSNKALPAPIEKTISKAKGQPREPQVYTLPPCRE |
| | | EMTKNQVSLWCLVKGFYPSDIAVEWESNGQPENNYKTTPPVLDSDGSFFLYS |
| | | KLTVDKSRWQQGNVFSCSVMHEALHNHYTQKSLSLSP |
| SEQ ID | NKp30 | GFSIIRVGYHWN |
| NO: 502 | VHCDR1 | |
| SEQ ID | NKp30 | YIHRSGSTSYNPSLKS |
| NO: 503 | VHCDR2 | |
| SEQ ID | NKp30 | GDWHYFDY |
| NO: 504 | VHCDR3 | |
| SEQ ID | NKp30 | SGEKLSDKYVH |
| NO: 26 | VLCDR1 | |
| SEQ ID | NKp30 | ENDRRPS |
| NO: 28 | VLCDR2 | |
| SEQ ID | NKp30 | QFWDSTNSAV |
| NO: 86 | VLCDR3 | |
| SEQ ID | NKp30 VH | EIQLLESGGGLVQPGGSLRLSCAVSGFSIIRVGYHWNWVRQAPGKGLEWVGY |
| NO: 505 | (BM130) | IHRSGSTSYNPSLKSRFTISRDTSKNTFYLQMNSLRAEDTAVYYCARGDWHYF |
| | | DYWGQGTMVTVSS |
| SEQ ID | NKp30 VL | DSVTTQSPLSLPVTLGQPASISCSGEKLSDKYVHWYQQRPGQSPRMLIYENDR |
| NO: 13 | (BM130) | RPSGVPDRFSGSNSGNDATLKISRVEAEDVGVYFCQFWDSTNSAVFGGGTKV |
| | | EIK |
| SEQ ID | Linker | GGGGGGGGSGGGGSGGGGS |
| NO: 8291 | | |
| SEQ ID | Nkp30 scFv | EIQLLESGGGLVQPGGSLRLSCAVSGFSIIRVGYHWNWVRQAPGKGLEWVGY |
| NO: 518 | | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | ATLKISRVEAEDVGVYFCQFWDSTNSAVFGGGTKVEIK |
| SEQ ID | Heavy chain | DKTHTCPPCPAPELLGGPSVFLFPPKPKDTLMISRTPEVTCVVVDVSHEDPEV |
| NO: 3447 | hinge-Fc | KFNWYVDGVEVHNAKTKPREEQYASTYRVVSVLTVLHQDWLNGKEYKCK |
| | region (CH2- | VSNKALPAPIEKTISKAKGQPREPQVCTLPPSREEMTKNQVSLSCAVKGFYPS |
| | CH3) Hole | DIAVEWESNGQPENNYKTTPPVLDSDGSFFLVSKLTVDKSRWQQGNVFSCSV |
| | | MHEALHNHYTQKSLSLSPGK |
| SEQ ID | Heavy chain | DKTHTCPPCPAPELLGGPSVFLFPPKPKDTLMISRTPEVTCVVVDVSHEDPEV |
| NO: 511 | hinge-Fc | KFNWYVDGVEVHNAKTKPREEQYASTYRVVSVLTVLHQDWLNGKEYKCK |
| | region (CH2- | VSNKALPAPIEKTISKAKGQPREPQVCTLPPSREEMTKNQVSLSCAVKGFYPS |
| | CH3) Hole | DIAVEWESNGQPENNYKTTPPVLDSDGSFFLVSKLTVDKSRWQQGNVFSCSV |
| | | MHEALHNHYTQKSLSLSP |
| SEQ ID | TRBC 1 | GYVMH |
| NO: 8643 | VHCDR1 | |
| SEQ ID | TRBC 1 | FINPYNDDIQSNERFRG |
| NO: 201 | VHCDR2 | |
| SEQ ID | TRBC 1 | GAGYNFDGAYRFFDF |
| NO: 202 | VHCDR3 | |
| SEQ ID | TRBC 1 | RSSQRLVHSNGNTYLH |
| NO: 223 | VLCDR1 | |
| SEQ ID | TRBC 1 | RVSNRFP |
| NO: 224 | VLCDR2 | |
| SEQ ID | TRBC 1 | SQSTHVPYT |
| NO: 225 | VLCDR3 | |
| SEQ ID | TRBC1 VH | QVQLVQSGAEVKKPGSSVKVSCKASGYTFTGYVMHWVRQAPGQGLEWMG |
| NO: 512 | | FINPYNDDIQSNERFRGRVTITSDKSTTTAYMELSSLRSEDTAVYYCARGAGY |
| | | NFDGAYRFFDFWGQGTLVTVSS |
| SEQ ID | TRBC1 VL | DVVMTQSPLSLPVTLGQPASISCRSSQRLVHSNGNTYLHWYQQRPGQSPRLLI |
| NO: 8308 | | YRVSNRFPGVPDRFSGSGSGTDFTLKISRVEAEDVGVYFCSQSTHVPYTFGGG |
| | | TKVEIK |
| SEQ ID | Light chain | RTVAAPSVFIFPPSDEQLKSGTASVVCLLNNFYPREAKVQWKVDNALQSGNS |
| NO: 3644 | constant | QESVTEQDSKDSTYSLSSTLTLSKADYEKHKVYACEVTHQGLSSPVTKSFNR |
| | region | GEC |
| SEQ ID | Heavy chain | ASTKGPSVFPLAPSSKSTSGGTAALGCLVKDYFPEPVTVSWNSGALTSGVHTF |
| NO: 513 | constant | PAVLQSSGLYSLSSVVTVPSSSLGTQTYICNVNHKPSNTKVDKRVEPKSCDKT |
| | region (CH1- | HTCPPCPAPELLGGPSVFLFPPKPKDTLMISRTPEVTCVVVDVSHEDPEVKFN |
| | CH2-CH3) | WYVDGVEVHNAKTKPREEQYASTYRVVSVLTVLHQDWLNGKEYKCKVSN |
| | Knob | KALPAPIEKTISKAKGQPREPQVYTLPPCREEMTKNQVSLWCLVKGFYPSDIA |
| | | VEWESNGQPENNYKTTPPVLDSDGSFFLYSKLTVDKSRWQQGNVFSCSVMH |
| | | EALHNHYTQKSLSLSPGK |
| SEQ ID | Heavy chain | ASTKGPSVFPLAPSSKSTSGGTAALGCLVKDYFPEPVTVSWNSGALTSGVHTF |
| NO: 514 | constant | PAVLQSSGLYSLSSVVTVPSSSLGTQTYICNVNHKPSNTKVDKRVEPKSCDKT |
| | region (CH1- | HTCPPCPAPELLGGPSVFLFPPKPKDTLMISRTPEVTCVVVDVSHEDPEVKFN |
| | CH2-CH3) | WYVDGVEVHNAKTKPREEQYASTYRVVSVLTVLHQDWLNGKEYKCKVSN |
| | Knob | KALPAPIEKTISKAKGQPREPQVYTLPPCREEMTKNQVSLWCLVKGFYPSDIA |
| | | VEWESNGQPENNYKTTPPVLDSDGSFFLYSKLTVDKSRWQQGNVFSCSVMH |
| | | EALHNHYTQKSLSLSP |
| SEQ ID | Heavy chain | EPKSCDKTHTCPPCPAPELLGGPSVFLFPPKPKDTLMISRTPEVTCVVVDVSHE |
| NO: 515 | hinge-Fc | DPEVKFNWYVDGVEVHNAKTKPREEQYASTYRVVSVLTVLHQDWLNGKEY |
| | region (CH2- | KCKVSNKALPAPIEKTISKAKGQPREPQVYTLPPCREEMTKNQVSLWCLVKG |
| | CH3) Knob | FYPSDIAVEWESNGQPENNYKTTPPVLDSDGSFFLYSKLTVDKSRWQQGNVF |
| | | SCSVMHEALHNHYTQKSLSLSPGK |
| SEQ ID | Heavy chain | EPKSCDKTHTCPPCPAPELLGGPSVFLFPPKPKDTLMISRTPEVTCVVVDVSHE |
| NO: 516 | hinge-Fc | DPEVKFNWYVDGVEVHNAKTKPREEQYASTYRVVSVLTVLHQDWLNGKEY |
| | region (CH2- | KCKVSNKALPAPIEKTISKAKGQPREPQVYTLPPCREEMTKNQVSLWCLVKG |
| | CH3) Knob | FYPSDIAVEWESNGQPENNYKTTPPVLDSDGSFFLYSKLTVDKSRWQQGNVF |
| | | SCSVMHEALHNHYTQKSLSLSP |

Exemplary molecule 6 Anti-TRBC2-NKp30

| | | |
|---|---|---|
| SEQ ID | NKp30 scFv- | EIQLLESGGGLVQPGGSLRLSCAVSGFSIIRVGYHWNWVRQAPGKGLEWVGY |
| NO: 509 | CH-Hole | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | NDATLKISRVEAEDVGVYFCQFWDSTNSAVFGGGTKVEIKDKTHTCPPCPAP |
| | | ELLGGPSVFLFPPKPKDTLMISRTPEVTCVVVDVSHEDPEVKFNWYVDGVEV |
| | | HNAKTKPREEQYASTYRVVSVLTVLHQDWLNGKEYKCKVSNKALPAPIEK |
| | | TISKAKGQPREPQVCTLPPSREEMTKNQVSLSCAVKGFYPSDIAVEWESNGQ |
| | | PENNYKTTPPVLDSDGSFFLVSKLTVDKSRWQQGNVFSCSVMHEALHNHYT |
| | | QKSLSLSPGK |
| SEQ ID | NKp30 scFv- | EIQLLESGGGLVQPGGSLRLSCAVSGFSIIRVGYHWNWVRQAPGKGLEWVGY |
| NO: 4704 | CH-Hole | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | NDATLKISRVEAEDVGVYFCQFWDSTNSAVFGGGTKVEIKDKTHTCPPCPAP |
| | | ELLGGPSVFLFPPKPKDTLMISRTPEVTCVVVDVSHEDPEVKFNWYVDGVEV |
| | | HNAKTKPREEQYASTYRVVSVLTVLHQDWLNGKEYKCKVSNKALPAPIEK |
| | | TISKAKGQPREPQVCTLPPSREEMTKNQVSLSCAVKGFYPSDIAVEWESNGQ |
| | | PENNYKTTPPVLDSDGSFFLVSKLTVDKSRWQQGNVFSCSVMHEALHNHYT |
| | | QKSLSLSP |
| SEQ ID | Light chain | DVVMTQSPLSLPVTPGEPASISCRSSQNLVHSNGRTYLQWYLQKPGQSPQLLI |
| NO: 7439 | (VL-CL) | YRVSNRFPGVPDRFSGSGSGTDFTLKISRVEAEDVGVYFCSQSTHVPYTFGGG |
| | | TKVEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSPVT |
| | | KSFNRGEC |
| SEQ ID | Heavy chain | QVQLVQSGAEVKKPGASVKVSCKASTSGFHGYPMHWVRQAPGQGLEWMGF |
| NO: 7438 | (TRBC2 VH- | INPYNDDIQSNFRFRGRVTMTSDKSTTTAYMELSSLRSEDTAVYYCARGNGK |
| | CH)-knob | WGDGAYRFFDFWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYASTYRVVS |
| | | VLTVLHQDWLNGKEYKCKVSNKALPAPIEKTISKAKGQPREPQVYTLPPCRE |
| | | EMTKNQVSLWCLVKGFYPSDIAVEWESNGQPENNYKTTPPVLDSDGSFFLYS |
| | | KLTVDKSRWQQGNVFSCSVMHEALHNHYTQKSLSLSPGK |
| SEQ ID | Heavy chain | QVQLVQSGAEVKKPGASVKVSCKASTSGFHGYPMHWVRQAPGQGLEWMGF |
| NO: 4680 | (TRBC2 VH- | INPYNDDIQSNFRFRGRVTMTSDKSTTTAYMELSSLRSEDTAVYYCARGNGK |
| | CH)-knob | WGDGAYRFFDFWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYASTYRVVS |
| | | VLTVLHQDWLNGKEYKCKVSNKALPAPIEKTISKAKGQPREPQVYTLPPCRE |
| | | EMTKNQVSLWCLVKGFYPSDIAVEWESNGQPENNYKTTPPVLDSDGSFFLYS |
| | | KLTVDKSRWQQGNVFSCSVMHEALHNHYTQKSLSLSP |
| SEQ ID | NKp30 | GFSIIRVGYHWN |
| NO: 502 | VHCDR1 | |
| SEQ ID | NKp30 | YIHRSGSTSYNPSLKS |
| NO: 503 | VHCDR2 | |
| SEQ ID | NKp30 | GDWHYFDY |
| NO: 504 | VHCDR3 | |
| SEQ ID | NKp30 | SGEKLSDKYVH |
| NO: 26 | VLCDR1 | |
| SEQ ID | NKp30 | ENDRRPS |
| NO: 28 | VLCDR2 | |
| SEQ ID | NKp30 | QFWDSTNSAV |
| NO: 86 | VLCDR3 | |
| SEQ ID | NKp30 VH | EIQLLESGGGLVQPGGSLRLSCAVSGFSIIRVGYHWNWVRQAPGKGLEWVGY |
| NO: 505 | (BM130) | IHRSGSTSYNPSLKSRFTISRDTSKNTFYLQMNSLRAEDTAVYYCARGDWHYF |
| | | DYWGQGTMVTVSS |
| SEQ ID | NKp30 VL | DSVTTQSPLSLPVTLGQPASISCSGEKLSDKYVHWYQQRPGQSPRMLIYENDR |
| NO: 13 | (BM130) | RPSGVPDRFSGSNSGNDATLKISRVEAEDVGVYFCQFWDSTNSAVFGGGTKV |
| | | EIK |
| SEQ ID | Linker | GGGGSGGGGSGGGGSGGGGS |
| NO: 8291 | | |
| SEQ ID | Nkp30 scFv | EIQLLESGGGLVQPGGSLRLSCAVSGFSIIRVGYHWNWVRQAPGKGLEWVGY |
| NO: 518 | | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | ATLKISRVEAEDVGVYFCQFWDSTNSAVFGGGTKVEIK |
| SEQ ID | Heavy chain | DKTHTCPPCPAPELLGGPSVFLFPPKPKDTLMISRTPEVTCVVVDVSHEDPEV |
| NO: 3447 | hinge-Fc | KFNWYVDGVEVHNAKTKPREEQYASTYRVVSVLTVLHQDWLNGKEYKCK |
| | region (CH2- | VSNKALPAPIEKTISKAKGQPREPQVCTLPPSREEMTKNQVSLSCAVKGFYPS |
| | CH3) Hole | DIAVEWESNGQPENNYKTTPPVLDSDGSFFLVSKLTVDKSRWQQGNVFSCSV |
| | | MHEALHNHYTQKSLSLSPGK |
| SEQ ID | Heavy chain | DKTHTCPPCPAPELLGGPSVFLFPPKPKDTLMISRTPEVTCVVVDVSHEDPEV |
| NO: 511 | hinge-Fc | KFNWYVDGVEVHNAKTKPREEQYASTYRVVSVLTVLHQDWLNGKEYKCK |
| | region (CH2- | VSNKALPAPIEKTISKAKGQPREPQVCTLPPSREEMTKNQVSLSCAVKGFYPS |
| | CH3) Hole | DIAVEWESNGQPENNYKTTPPVLDSDGSFFLVSKLTVDKSRWQQGNVFSCSV |
| | | MHEALHNHYTQKSLSLSP |
| SEQ ID | TRBC 2 | TSGFHGYPMH |
| NO: 8543 | VHCDR1 | |
| SEQ ID | TRBC 2 | FINPYNDDIQSNERFRG |
| NO: 201 | VHCDR2 | |
| SEQ ID | TRBC 2 | GNGKWGDGAYRFFDF |
| NO: 7403 | VHCDR3 | |
| SEQ ID | TRBC 2 | RSSQNLVHSNGRTYLQ |
| NO: 7410 | VLCDR1 | |
| SEQ ID | TRBC 2 | RVSNRFP |
| NO: 224 | VLCDR2 | |
| SEQ ID | TRBC 2 | SQSTHVPYT |
| NO: 225 | VLCDR3 | |
| SEQ ID | TRBC2 VH | QVQLVQSGAEVKKPGASVKVSCKASTSGFHGYPMHWVRQAPGQGLEWMGF |
| NO: 7420 | | INPYNDDIQSNFRFRGRVTMTSDKSTTTAYMELSSLRSEDTAVYYCARGNGK |
| | | WGDGAYRFFDFWGQGTLVTVSS |
| SEQ ID | TRBC2 VL | DVVMTQSPLSLPVTPGEPASISCRSSQNLVHSNGRTYLQWYLQKPGQSPQLLI |
| NO: 7419 | | YRVSNRFPGVPDRFSGSGSGTDFTLKISRVEAEDVGVYFCSQSTHVPYTFGGG |
| | | TKVEIK |
| SEQ ID | Light chain | RTVAAPSVFIFPPSDEQLKSGTASVVCLLNNFYPREAKVQWKVDNALQSGNS |
| NO: 3644 | constant | QESVTEQDSKDSTYSLSSTLTLSKADYEKHKVYACEVTHQGLSSPVTKSFNR |
| | region | GEC |
| SEQ ID | Heavy chain | ASTKGPSVFPLAPSSKSTSGGTAALGCLVKDYFPEPVTVSWNSGALTSGVHTF |
| NO: 513 | constant | PAVLQSSGLYSLSSVVTVPSSSLGTQTYICNVNHKPSNTKVDKRVEPKSCDKT |
| | region (CH1- | HTCPPCPAPELLGGPSVFLFPPKPKDTLMISRTPEVTCVVVDVSHEDPEVKFN |
| | CH2-CH3) | WYVDGVEVHNAKTKPREEQYASTYRVVSVLTVLHQDWLNGKEYKCKVSN |
| | Knob | KALPAPIEKTISKAKGQPREPQVYTLPPCREEMTKNQVSLWCLVKGFYPSDIA |
| | | VEWESNGQPENNYKTTPPVLDSDGSFFLYSKLTVDKSRWQQGNVFSCSVMH |
| | | EALHNHYTQKSLSLSPGK |
| SEQ ID | Heavy chain | ASTKGPSVFPLAPSSKSTSGGTAALGCLVKDYFPEPVTVSWNSGALTSGVHTF |
| NO: 514 | constant | PAVLQSSGLYSLSSVVTVPSSSLGTQTYICNVNHKPSNTKVDKRVEPKSCDKT |
| | region (CH1- | HTCPPCPAPELLGGPSVFLFPPKPKDTLMISRTPEVTCVVVDVSHEDPEVKFN |
| | CH2-CH3) | WYVDGVEVHNAKTKPREEQYASTYRVVSVLTVLHQDWLNGKEYKCKVSN |
| | Knob | KALPAPIEKTISKAKGQPREPQVYTLPPCREEMTKNQVSLWCLVKGFYPSDIA |
| | | VEWESNGQPENNYKTTPPVLDSDGSFFLYSKLTVDKSRWQQGNVFSCSVMH |
| | | EALHNHYTQKSLSLSP |
| SEQ ID | Heavy chain | EPKSCDKTHTCPPCPAPELLGGPSVFLFPPKPKDTLMISRTPEVTCVVVDVSHE |
| NO: 515 | hinge-Fc | DPEVKFNWYVDGVEVHNAKTKPREEQYASTYRVVSVLTVLHQDWLNGKEY |
| | region (CH2- | KCKVSNKALPAPIEKTISKAKGQPREPQVYTLPPCREEMTKNQVSLWCLVKG |
| | CH3) Knob | FYPSDIAVEWESNGQPENNYKTTPPVLDSDGSFFLYSKLTVDKSRWQQGNVF |
| | | SCSVMHEALHNHYTQKSLSLSPGK |
| SEQ ID | Heavy chain | EPKSCDKTHTCPPCPAPELLGGPSVFLFPPKPKDTLMISRTPEVTCVVVDVSHE |
| NO: 516 | hinge-Fc | DPEVKFNWYVDGVEVHNAKTKPREEQYASTYRVVSVLTVLHQDWLNGKEY |
| | region (CH2- | KCKVSNKALPAPIEKTISKAKGQPREPQVYTLPPCREEMTKNQVSLWCLVKG |
| | CH3) Knob | FYPSDIAVEWESNGQPENNYKTTPPVLDSDGSFFLYSKLTVDKSRWQQGNVF |
| | | SCSVMHEALHNHYTQKSLSLSP |

Exemplary molecule 7 Anti-TRBC2-NKp30

| | | |
|---|---|---|
| SEQ ID | NKp30 scFv- | EIQLLESGGGLVQPGGSLRLSCAVSGFSIIRVGYHWNWVRQAPGKGLEWVGY |
| NO: 509 | CH-Hole | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | ATLKISRVEAEDVGVYFCQFWDSTNSAVFGGGTKVEIKDKTHTCPPCPAPELL |
| | | GGPSVFLFPPKPKDTLMISRTPEVTCVVVDVSHEDPEVKFNWYVDGVEVHNA |
| | | KTKPREEQYASTYRVVSVLTVLHQDWLNGKEYKCKVSNKALPAPIEKTISKA |
| | | KGQPREPQVCTLPPSREEMTKNQVSLSCAVKGFYPSDIAVEWESNGQPENNY |
| | | KTTPPVLDSDGSFFLVSKLTVDKSRWQQGNVFSCSVMHEALHNHYTQKSLSL |
| | | SPGK |
| SEQ ID | NKp30 scFv- | EIQLLESGGGLVQPGGSLRLSCAVSGFSIIRVGYHWNWVRQAPGKGLEWVGY |
| NO: 4704 | CH-Hole | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | ATLKISRVEAEDVGVYFCQFWDSTNSAVFGGGTKVEIKDKTHTCPPCPAPELL |
| | | GGPSVFLFPPKPKDTLMISRTPEVTCVVVDVSHEDPEVKFNWYVDGVEVHNA |
| | | KTKPREEQYASTYRVVSVLTVLHQDWLNGKEYKCKVSNKALPAPIEKTISKA |
| | | KGQPREPQVCTLPPSREEMTKNQVSLSCAVKGFYPSDIAVEWESNGQPENNY |
| | | KTTPPVLDSDGSFFLVSKLTVDKSRWQQGNVFSCSVMHEALHNHYTQKSLSL |
| | | SP |
| SEQ ID | Light chain | DVVMTQSPLSLPVTPGEPASISCRSSQNLVHSNGRTYLQWYLQKPGQSPQLLI |
| NO: 7439 | (VL-CL) | YRVSNRFPGVPDRFSGSGSGTDFTLKISRVEAEDVGVYFCSQSTHVPYTFGGG |
| | | TKVEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSPVT |
| | | KSFNRGEC |
| SEQ ID | Heavy chain | QVQLVQSGAEVKKPGASVKVSCKASPRGFHGYHMHWVRQAPGQGLEWMG |
| NO: 7440 | (TRBC2 VH- | FINPYNDDIQSNERFRGRVTMTSDKSTTTAYMELSSLRSEDTAVYYCARGNG |
| | CH)-knob | KWGDGAYRFFDFWGQGTLVTVSSASTKGPSVFPLAPSSKSTSGGTAALGCLV |
| | | KDYFPEPVTVSWNSGALTSGVHTFPAVLQSSGLYSLSSVVTVPSSSLGTQTYI |
| | | CNVNHKPSNTKVDKRVEPKSCDKTHTCPPCPAPELLGGPSVFLFPPKPKDTLM |
| | | ISRTPEVTCVVVDVSHEDPEVKFNWYVDGVEVHNAKTKPREEQYASTYRVV |
| | | SVLTVLHQDWLNGKEYKCKVSNKALPAPIEKTISKAKGQPREPQVYTLPPCR |
| | | EEMTKNQVSLWCLVKGFYPSDIAVEWESNGQPENNYKTTPPVLDSDGSFFLY |
| | | SKLTVDKSRWQQGNVFSCSVMHEALHNHYTQKSLSLSPGK |
| SEQ ID | Heavy chain | QVQLVQSGAEVKKPGASVKVSCKASPRGFHGYHMHWVRQAPGQGLEWMG |
| NO: 4681 | (TRBC2 VH- | FINPYNDDIQSNERFRGRVTMTSDKSTTTAYMELSSLRSEDTAVYYCARGNG |
| | CH)-knob | KWGDGAYRFFDFWGQGTLVTVSSASTKGPSVFPLAPSSKSTSGGTAALGCLV |
| | | KDYFPEPVTVSWNSGALTSGVHTFPAVLQSSGLYSLSSVVTVPSSSLGTQTYI |
| | | CNVNHKPSNTKVDKRVEPKSCDKTHTCPPCPAPELLGGPSVFLFPPKPKDTLM |
| | | ISRTPEVTCVVVDVSHEDPEVKFNWYVDGVEVHNAKTKPREEQYASTYRVV |
| | | SVLTVLHQDWLNGKEYKCKVSNKALPAPIEKTISKAKGQPREPQVYTLPPCR |
| | | EEMTKNQVSLWCLVKGFYPSDIAVEWESNGQPENNYKTTPPVLDSDGSFFLY |
| | | SKLTVDKSRWQQGNVFSCSVMHEALHNHYTQKSLSLSP |
| SEQ ID | NKp30 | GFSIIRVGYHWN |
| NO: 502 | VHCDR1 | |
| SEQ ID | NKp30 | YIHRSGSTSYNPSLKS |
| NO: 503 | VHCDR2 | |
| SEQ ID | NKp30 | GDWHYFDY |
| NO: 504 | VHCDR3 | |
| SEQ ID | NKp30 | SGEKLSDKYVH |
| NO: 26 | VLCDR1 | |
| SEQ ID | NKp30 | ENDRRPS |
| NO: 28 | VLCDR2 | |
| SEQ ID | NKp30 | QFWDSTNSAV |
| NO: 86 | VLCDR3 | |
| SEQ ID | NKp30 VH | EIQLLESGGGLVQPGGSLRLSCAVSGFSIIRVGYHWNWVRQAPGKGLEWVGY |
| NO: 505 | (BM130) | IHRSGSTSYNPSLKSRFTISRDTSKNTFYLQMNSLRAEDTAVYYCARGDWHYF |
| | | DYWGQGTMVTVSS |
| SEQ ID | NKp30 VL | DSVTTQSPLSLPVTLGQPASISCSGEKLSDKYVHWYQQRPGQSPRMLIYENDR |
| NO: 13 | (BM130) | RPSGVPDRFSGSNSGNDATLKISRVEAEDVGVYFCQFWDSTNSAVFGGGTKV |
| | | EIK |
| SEQ ID | Linker | GGGGSGGGGSGGGGSGGGGS |
| NO: 8291 | | |
| SEQ ID | Nkp30 scFv | EIQLLESGGGLVQPGGSLRLSCAVSGFSIIRVGYHWNWVRQAPGKGLEWVGY |
| NO: 518 | | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | ATLKISRVEAEDVGVYFCQFWDSTNSAVFGGGTKVEIK |
| SEQ ID | Heavy chain | DKTHTCPPCPAPELLGGPSVFLFPPKPKDTLMISRTPEVTCVVVDVSHEDPEV |
| NO: 3447 | hinge-Fc | KFNWYVDGVEVHNAKTKPREEQYASTYRVVSVLTVLHQDWLNGKEYKCK |
| | region (CH2- | VSNKALPAPIEKTISKAKGQPREPQVCTLPPSREEMTKNQVSLSCAVKGFYPS |
| | CH3) Hole | DIAVEWESNGQPENNYKTTPPVLDSDGSFFLVSKLTVDKSRWQQGNVFSCSV |
| | | MHEALHNHYTQKSLSLSPGK |
| SEQ ID | Heavy chain | DKTHTCPPCPAPELLGGPSVFLFPPKPKDTLMISRTPEVTCVVVDVSHEDPEV |
| NO: 511 | hinge-Fc | KFNWYVDGVEVHNAKTKPREEQYASTYRVVSVLTVLHQDWLNGKEYKCK |
| | region (CH2- | VSNKALPAPIEKTISKAKGQPREPQVCTLPPSREEMTKNQVSLSCAVKGFYPS |
| | CH3) Hole | DIAVEWESNGQPENNYKTTPPVLDSDGSFFLVSKLTVDKSRWQQGNVFSCSV |
| | | MHEALHNHYTQKSLSLSP |
| SEQ ID | TRBC 2 | GYHMH |
| NO: 7401 | VHCDR1 | |
| SEQ ID | TRBC 2 | FINPYNDDIQSNERFRG |
| NO: 201 | VHCDR2 | |
| SEQ ID | TRBC 2 | GNGKWGDGAYRFFDF |
| NO: 7403 | VHCDR3 | |
| SEQ ID | TRBC 2 | RSSQNLVHSNGRTYLQ |
| NO: 7410 | VLCDR1 | |
| SEQ ID | TRBC 2 | RVSNRFP |
| NO: 224 | VLCDR2 | |
| SEQ ID | TRBC 2 | SQSTHVPYT |
| NO: 225 | VLCDR3 | |
| SEQ ID | TRBC2 VH | QVQLVQSGAEVKKPGASVKVSCKASPRGFHGYHMHWVRQAPGQGLEWMG |
| NO: 7423 | | FINPYNDDIQSNERFRGRVTMTSDKSTTTAYMELSSLRSEDTAVYYCARGNG |
| | | KWGDGAYRFFDFWGQGTLVTVSS |
| SEQ ID | TRBC2 VL | DVVMTQSPLSLPVTPGEPASISCRSSQNLVHSNGRTYLQWYLQKPGQSPQLLI |
| NO: 7419 | | YRVSNRFPGVPDRFSGSGSGTDFTLKISRVEAEDVGVYFCSQSTHVPYTFGGG |
| | | TKVEIK |
| SEQ ID | Light chain | RTVAAPSVFIFPPSDEQLKSGTASVVCLLNNFYPREAKVQWKVDNALQSGNS |
| NO: 3644 | constant | QESVTEQDSKDSTYSLSSTLTLSKADYEKHKVYACEVTHQGLSSPVTKSFNR |
| | region | GEC |
| SEQ ID | Heavy chain | ASTKGPSVFPLAPSSKSTSGGTAALGCLVKDYFPEPVTVSWNSGALTSGVHTF |
| NO: 513 | constant | PAVLQSSGLYSLSSVVTVPSSSLGTQTYICNVNHKPSNTKVDKRVEPKSCDKT |
| | region (CH1- | HTCPPCPAPELLGGPSVFLFPPKPKDTLMISRTPEVTCVVVDVSHEDPEVKFN |
| | CH2-CH3) | WYVDGVEVHNAKTKPREEQYASTYRVVSVLTVLHQDWLNGKEYKCKVSN |
| | Knob | KALPAPIEKTISKAKGQPREPQVYTLPPCREEMTKNQVSLWCLVKGFYPSDIA |
| | | VEWESNGQPENNYKTTPPVLDSDGSFFLYSKLTVDKSRWQQGNVFSCSVMH |
| | | EALHNHYTQKSLSLSPGK |
| SEQ ID | Heavy chain | ASTKGPSVFPLAPSSKSTSGGTAALGCLVKDYFPEPVTVSWNSGALTSGVHTF |
| NO: 514 | constant | PAVLQSSGLYSLSSVVTVPSSSLGTQTYICNVNHKPSNTKVDKRVEPKSCDKT |
| | region (CH1- | HTCPPCPAPELLGGPSVFLFPPKPKDTLMISRTPEVTCVVVDVSHEDPEVKFN |
| | CH2-CH3) | WYVDGVEVHNAKTKPREEQYASTYRVVSVLTVLHQDWLNGKEYKCKVSN |
| | Knob | KALPAPIEKTISKAKGQPREPQVYTLPPCREEMTKNQVSLWCLVKGFYPSDIA |
| | | VEWESNGQPENNYKTTPPVLDSDGSFFLYSKLTVDKSRWQQGNVFSCSVMH |
| | | EALHNHYTQKSLSLSP |
| SEQ ID | Heavy chain | EPKSCDKTHTCPPCPAPELLGGPSVFLFPPKPKDTLMISRTPEVTCVVVDVSHE |
| NO: 515 | hinge-Fc | DPEVKFNWYVDGVEVHNAKTKPREEQYASTYRVVSVLTVLHQDWLNGKEY |
| | region (CH2- | KCKVSNKALPAPIEKTISKAKGQPREPQVYTLPPCREEMTKNQVSLWCLVKG |
| | CH3) Knob | FYPSDIAVEWESNGQPENNYKTTPPVLDSDGSFFLYSKLTVDKSRWQQGNVF |
| | | SCSVMHEALHNHYTQKSLSLSPGK |
| SEQ ID | Heavy chain | EPKSCDKTHTCPPCPAPELLGGPSVFLFPPKPKDTLMISRTPEVTCVVVDVSHE |
| NO: 516 | hinge-Fc | DPEVKFNWYVDGVEVHNAKTKPREEQYASTYRVVSVLTVLHQDWLNGKEY |
| | region (CH2- | KCKVSNKALPAPIEKTISKAKGQPREPQVYTLPPCREEMTKNQVSLWCLVKG |
| | CH3) Knob | FYPSDIAVEWESNGQPENNYKTTPPVLDSDGSFFLYSKLTVDKSRWQQGNVF |
| | | SCSVMHEALHNHYTQKSLSLSP |

Exemplary molecule 8 Anti-TRBC2-NKp30

| | | |
|---|---|---|
| SEQ ID | NKp30 scFv- | EIQLLESGGGLVQPGGSLRLSCAVSGFSIIRVGYHWNWVRQAPGKGLEWVGY |
| NO: 509 | CH-Hole | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | ATLKISRVEAEDVGVYFCQFWDSTNSAVFGGGTKVEIKDKTHTCPPCPAPELL |
| | | GGPSVFLFPPKPKDTLMISRTPEVTCVVVDVSHEDPEVKFNWYVDGVEVHNA |
| | | KTKPREEQYASTYRVVSVLTVLHQDWLNGKEYKCKVSNKALPAPIEKTISKA |
| | | KGQPREPQVCTLPPSREEMTKNQVSLSCAVKGFYPSDIAVEWESNGQPENNY |
| | | KTTPPVLDSDGSFFLVSKLTVDKSRWQQGNVFSCSVMHEALHNHYTQKSLSL |
| | | SPGK |
| SEQ ID | NKp30 scFv- | EIQLLESGGGLVQPGGSLRLSCAVSGFSIIRVGYHWNWVRQAPGKGLEWVGY |
| NO: 4704 | CH-Hole | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | ATLKISRVEAEDVGVYFCQFWDSTNSAVFGGGTKVEIKDKTHTCPPCPAPELL |
| | | GGPSVFLFPPKPKDTLMISRTPEVTCVVVDVSHEDPEVKFNWYVDGVEVHNA |
| | | KTKPREEQYASTYRVVSVLTVLHQDWLNGKEYKCKVSNKALPAPIEKTISKA |
| | | KGQPREPQVCTLPPSREEMTKNQVSLSCAVKGFYPSDIAVEWESNGQPENNY |
| | | KTTPPVLDSDGSFFLVSKLTVDKSRWQQGNVFSCSVMHEALHNHYTQKSLSL |
| | | SP |
| SEQ ID | Light chain | DVVMTQSPLSLPVTLGQPASISCRSSENLVHSNGRTYLQWYQQRPGQSPRLLI |
| NO: 8002 | (VL-CL) | YRVSNRFPGVPDRFSGSGSGTDFTLKISRVEAEDVGVYFCSQSSLEPYTFGGG |
| | | TKVEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSPVT |
| | | KSFNRGEC |
| SEQ ID | Heavy chain | QVQLVQSGAEVKKPGSSVKVSCKASPRGFYGYHMHWVRQAPGQGLEWMG |
| NO: 8001 | (TRBC2 VH- | FINPYTNDIQYNERFRGRVTITSDESTTTAYMELSSLRSEDTAVYYCAMGNGK |
| | CH)-knob | WGDGAYRFFDLWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYASTYRVVS |
| | | VLTVLHQDWLNGKEYKCKVSNKALPAPIEKTISKAKGQPREPQVYTLPPCRE |
| | | EMTKNQVSLWCLVKGFYPSDIAVEWESNGQPENNYKTTPPVLDSDGSFFLYS |
| | | KLTVDKSRWQQGNVFSCSVMHEALHNHYTQKSLSLSPGK |
| SEQ ID | Heavy chain | QVQLVQSGAEVKKPGSSVKVSCKASPRGFYGYHMHWVRQAPGQGLEWMG |
| NO: 4682 | (TRBC2 VH- | FINPYTNDIQYNERFRGRVTITSDESTTTAYMELSSLRSEDTAVYYCAMGNGK |
| | CH)-knob | WGDGAYRFFDLWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYASTYRVVS |
| | | VLTVLHQDWLNGKEYKCKVSNKALPAPIEKTISKAKGQPREPQVYTLPPCRE |
| | | EMTKNQVSLWCLVKGFYPSDIAVEWESNGQPENNYKTTPPVLDSDGSFFLYS |
| | | KLTVDKSRWQQGNVFSCSVMHEALHNHYTQKSLSLSP |
| SEQ ID | NKp30 | GFSIIRVGYHWN |
| NO: 502 | VHCDR1 | |
| SEQ ID | NKp30 | YIHRSGSTSYNPSLKS |
| NO: 503 | VHCDR2 | |
| SEQ ID | NKp30 | GDWHYFDY |
| NO: 504 | VHCDR3 | |
| SEQ ID | NKp30 | SGEKLSDKYVH |
| NO: 26 | VLCDR1 | |
| SEQ ID | NKp30 | ENDRRPS |
| NO: 28 | VLCDR2 | |
| SEQ ID | NKp30 | QFWDSTNSAV |
| NO: 86 | VLCDR3 | |
| SEQ ID | NKp30 VH | EIQLLESGGGLVQPGGSLRLSCAVSGFSIIRVGYHWNWVRQAPGKGLEWVGY |
| NO: 505 | (BM130) | IHRSGSTSYNPSLKSRFTISRDTSKNTFYLQMNSLRAEDTAVYYCARGDWHYF |
| | | DYWGQGTMVTVSS |
| SEQ ID | NKp30 VL | DSVTTQSPLSLPVTLGQPASISCSGEKLSDKYVHWYQQRPGQSPRMLIYENDR |
| NO: 13 | (BM130) | RPSGVPDRFSGSNSGNDATLKISRVEAEDVGVYFCQFWDSTNSAVFGGGTKV |
| | | EIK |
| SEQ ID | Linker | GGGGSGGGGSGGGGSGGGGS |
| NO: 8291 | | |
| SEQ ID | Nkp30 scFv | EIQLLESGGGLVQPGGSLRLSCAVSGFSIIRVGYHWNWVRQAPGKGLEWVGY |
| NO: 518 | | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | ATLKISRVEAEDVGVYFCQFWDSTNSAVFGGGTKVEIK |
| SEQ ID | Heavy chain | DKTHTCPPCPAPELLGGPSVFLFPPKPKDTLMISRTPEVTCVVVDVSHEDPEV |
| NO: 3447 | hinge-Fc | KFNWYVDGVEVHNAKTKPREEQYASTYRVVSVLTVLHQDWLNGKEYKCK |
| | region (CH2- | VSNKALPAPIEKTISKAKGQPREPQVCTLPPSREEMTKNQVSLSCAVKGFYPS |
| | CH3) Hole | DIAVEWESNGQPENNYKTTPPVLDSDGSFFLVSKLTVDKSRWQQGNVFSCSV |
| | | MHEALHNHYTQKSLSLSPGK |
| SEQ ID | Heavy chain | DKTHTCPPCPAPELLGGPSVFLFPPKPKDTLMISRTPEVTCVVVDVSHEDPEV |
| NO: 511 | hinge-Fc | KFNWYVDGVEVHNAKTKPREEQYASTYRVVSVLTVLHQDWLNGKEYKCK |
| | region (CH2- | VSNKALPAPIEKTISKAKGQPREPQVCTLPPSREEMTKNQVSLSCAVKGFYPS |
| | CH3) Hole | DIAVEWESNGQPENNYKTTPPVLDSDGSFFLVSKLTVDKSRWQQGNVFSCSV |
| | | MHEALHNHYTQKSLSLSP |
| SEQ ID | TRBC 2 | PRGFYGYHMH |
| NO: | VHCDR1 | |
| 8272 | | |
| SEQ ID | TRBC 2 | FINPYTNDIQYNERFRG |
| NO: 8260 | VHCDR2 | |
| SEQ ID | TRBC 2 | GNGKWGDGAYRFFDL |
| NO: 8227 | VHCDR3 | |
| SEQ ID | TRBC 2 | RSSENLVHSNGRTYLQ |
| NO: 8048 | VLCDR1 | |
| SEQ ID | TRBC 2 | RVSNRFP |
| NO: 224 | VLCDR2 | |
| SEQ ID | TRBC 2 | SQSSLEPYT |
| NO: 8050 | VLCDR3 | |
| SEQ ID | TRBC2 VH | QVQLVQSGAEVKKPGSSVKVSCKASPRGFYGYHMHWVRQAPGQGLEWMG |
| NO: 8011 | | FINPYTNDIQYNERFRGRVTITSDESTTTAYMELSSLRSEDTAVYYCAMGNGK |
| | | WGDGAYRFFDLWGQGTLVTVSS |
| SEQ ID | TRBC2 VL | DVVMTQSPLSLPVTLGQPASISCRSSENLVHSNGRTYLQWYQQRPGQSPRLLI |
| NO: 8012 | | YRVSNRFPGVPDRFSGSGSGTDFTLKISRVEAEDVGVYFCSQSSLEPYTFGGG |
| | | TKVEIK |
| SEQ ID | Light chain | RTVAAPSVFIFPPSDEQLKSGTASVVCLLNNFYPREAKVQWKVDNALQSGNS |
| NO: 3644 | constant | QESVTEQDSKDSTYSLSSTLTLSKADYEKHKVYACEVTHQGLSSPVTKSFNR |
| | region | GEC |
| SEQ ID | Heavy chain | ASTKGPSVFPLAPSSKSTSGGTAALGCLVKDYFPEPVTVSWNSGALTSGVHTF |
| NO: 513 | constant | PAVLQSSGLYSLSSVVTVPSSSLGTQTYICNVNHKPSNTKVDKRVEPKSCDKT |
| | region (CH1- | HTCPPCPAPELLGGPSVFLFPPKPKDTLMISRTPEVTCVVVDVSHEDPEVKFN |
| | CH2-CH3) | WYVDGVEVHNAKTKPREEQYASTYRVVSVLTVLHQDWLNGKEYKCKVSN |
| | Knob | KALPAPIEKTISKAKGQPREPQVYTLPPCREEMTKNQVSLWCLVKGFYPSDIA |
| | | VEWESNGQPENNYKTTPPVLDSDGSFFLYSKLTVDKSRWQQGNVFSCSVMH |
| | | EALHNHYTQKSLSLSPGK |
| SEQ ID | Heavy chain | ASTKGPSVFPLAPSSKSTSGGTAALGCLVKDYFPEPVTVSWNSGALTSGVHTF |
| NO: 514 | constant | PAVLQSSGLYSLSSVVTVPSSSLGTQTYICNVNHKPSNTKVDKRVEPKSCDKT |
| | region (CH1- | HTCPPCPAPELLGGPSVFLFPPKPKDTLMISRTPEVTCVVVDVSHEDPEVKFN |
| | CH2-CH3) | WYVDGVEVHNAKTKPREEQYASTYRVVSVLTVLHQDWLNGKEYKCKVSN |
| | Knob | KALPAPIEKTISKAKGQPREPQVYTLPPCREEMTKNQVSLWCLVKGFYPSDIA |
| | | VEWESNGQPENNYKTTPPVLDSDGSFFLYSKLTVDKSRWQQGNVFSCSVMH |
| | | EALHNHYTQKSLSLSP |
| SEQ ID | Heavy chain | EPKSCDKTHTCPPCPAPELLGGPSVFLFPPKPKDTLMISRTPEVTCVVVDVSHE |
| NO: 515 | hinge-Fc | DPEVKFNWYVDGVEVHNAKTKPREEQYASTYRVVSVLTVLHQDWLNGKEY |
| | region (CH2- | KCKVSNKALPAPIEKTISKAKGQPREPQVYTLPPCREEMTKNQVSLWCLVKG |
| | CH3) Knob | FYPSDIAVEWESNGQPENNYKTTPPVLDSDGSFFLYSKLTVDKSRWQQGNVF |
| | | SCSVMHEALHNHYTQKSLSLSPGK |
| SEQ ID | Heavy chain | EPKSCDKTHTCPPCPAPELLGGPSVFLFPPKPKDTLMISRTPEVTCVVVDVSHE |
| NO: 516 | hinge-Fc | DPEVKFNWYVDGVEVHNAKTKPREEQYASTYRVVSVLTVLHQDWLNGKEY |
| | region (CH2- | KCKVSNKALPAPIEKTISKAKGQPREPQVYTLPPCREEMTKNQVSLWCLVKG |
| | CH3) Knob | FYPSDIAVEWESNGQPENNYKTTPPVLDSDGSFFLYSKLTVDKSRWQQGNVF |
| | | SCSVMHEALHNHYTQKSLSLSP |

Exemplary Molecule 9 Anti-TRBC2-NKp30

| | | |
|---|---|---|
| SEQ ID | NKp30 scFv- | EIQLLESGGGLVQPGGSLRLSCAVSGFSIIRVGYHWNWVRQAPGKGLEWVGY |
| NO: | CH-Hole | IHRSGSTSYNPSLKSRFTISRDTSKNTFYLQMNSLRAEDTAVYYCARGDWHYF |
| 509 | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | ATLKISRVEAEDVGVYFCQFWDSTNSAVFGGGTKVEIKDKTHTCPPCPAPELL |
| | | GGPSVFLFPPKPKDTLMISRTPEVTCVVVDVSHEDPEVKFNWYVDGVEVHNA |
| | | KTKPREEQYASTYRVVSVLTVLHQDWLNGKEYKCKVSNKALPAPIEKTISKA |
| | | KGQPREPQVCTLPPSREEMTKNQVSLSCAVKGFYPSDIAVEWESNGQPENNY |
| | | KTTPPVLDSDGSFFLVSKLTVDKSRWQQGNVFSCSVMHEALHNHYTQKSLSL |
| | | SPGK |
| SEQ ID | NKp30 scFv- | EIQLLESGGGLVQPGGSLRLSCAVSGFSIIRVGYHWNWVRQAPGKGLEWVGY |
| NO: 4704 | CH-Hole | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | ATLKISRVEAEDVGVYFCQFWDSTNSAVFGGGTKVEIKDKTHTCPPCPAPELL |
| | | GGPSVFLFPPKPKDTLMISRTPEVTCVVVDVSHEDPEVKFNWYVDGVEVHNA |
| | | KTKPREEQYASTYRVVSVLTVLHQDWLNGKEYKCKVSNKALPAPIEKTISKA |
| | | KGQPREPQVCTLPPSREEMTKNQVSLSCAVKGFYPSDIAVEWESNGQPENNY |
| | | KTTPPVLDSDGSFFLVSKLTVDKSRWQQGNVFSCSVMHEALHNHYTQKSLSL |
| | | SP |
| SEQ ID | Light chain | DVVMTQSPLSLPVTLGQPASISCRSSKNLVHSNGRTYLQWYQQRPGQSPRLLI |
| NO: 8005 | (VL-CL) | YRVSNRFPGVPDRFSGSGSGTDFTLKISRVEAEDVGVYFCSQSTREPYTFGGG |
| | | TKVEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSPVT |
| | | KSFNRGEC |
| SEQ ID | Heavy chain | QVQLVQSGAEVKKPGSSVKVSCKASPRGFYGYHMHWVRQAPGQGLEWMG |
| NO: 8004 | (TRBC2 VH- | FINPYNNHIQYNERFRGRVTITSDESTTTAYMELSSLRSEDTAVYYCALGNGK |
| | CH)-knob | WGDGAYRFFDFWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYASTYRVVS |
| | | VLTVLHQDWLNGKEYKCKVSNKALPAPIEKTISKAKGQPREPQVYTLPPCRE |
| | | EMTKNQVSLWCLVKGFYPSDIAVEWESNGQPENNYKTTPPVLDSDGSFFLYS |
| | | KLTVDKSRWQQGNVFSCSVMHEALHNHYTQKSLSLSPGK |
| SEQ ID | Heavy chain | QVQLVQSGAEVKKPGSSVKVSCKASPRGFYGYHMHWVRQAPGQGLEWMG |
| NO: 4685 | (TRBC2 VH- | FINPYNNHIQYNERFRGRVTITSDESTTTAYMELSSLRSEDTAVYYCALGNGK |
| | CH)-knob | WGDGAYRFFDFWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYASTYRVVS |
| | | VLTVLHQDWLNGKEYKCKVSNKALPAPIEKTISKAKGQPREPQVYTLPPCRE |
| | | EMTKNQVSLWCLVKGFYPSDIAVEWESNGQPENNYKTTPPVLDSDGSFFLYS |
| | | KLTVDKSRWQQGNVFSCSVMHEALHNHYTQKSLSLSP |
| SEQ ID | NKp30 | GFSIIRVGYHWN |
| NO: 502 | VHCDR1 | |
| SEQ ID | NKp30 | YIHRSGSTSYNPSLKS |
| NO: 503 | VHCDR2 | |
| SEQ ID | NKp30 | GDWHYFDY |
| NO: 504 | VHCDR3 | |
| SEQ ID | NKp30 | SGEKLSDKYVH |
| NO: 26 | VLCDR1 | |
| SEQ ID | NKp30 | ENDRRPS |
| NO: 28 | VLCDR2 | |
| SEQ ID | NKp30 | QFWDSTNSAV |
| NO: 86 | VLCDR3 | |
| SEQ ID | NKp30 VH | EIQLLESGGGLVQPGGSLRLSCAVSGFSIIRVGYHWNWVRQAPGKGLEWVGY |
| NO: 505 | (BM130) | IHRSGSTSYNPSLKSRFTISRDTSKNTFYLQMNSLRAEDTAVYYCARGDWHYF |
| | | DYWGQGTMVTVSS |
| SEQ ID | NKp30 VL | DSVTTQSPLSLPVTLGQPASISCSGEKLSDKYVHWYQQRPGQSPRMLIYENDR |
| NO: 13 | (BM130) | RPSGVPDRFSGSNSGNDATLKISRVEAEDVGVYFCQFWDSTNSAVFGGGTKV |
| | | EIK |
| SEQ ID | Linker | GGGGSGGGGSGGGGSGGGGS |
| NO: 8291 | | |
| SEQ ID | Nkp30 scFv | EIQLLESGGGLVQPGGSLRLSCAVSGFSIIRVGYHWNWVRQAPGKGLEWVGY |
| NO: 518 | | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | ATLKISRVEAEDVGVYFCQFWDSTNSAVFGGGTKVEIK |
| SEQ ID | Heavy chain | DKTHTCPPCPAPELLGGPSVFLFPPKPKDTLMISRTPEVTCVVVDVSHEDPEV |
| NO: 3447 | hinge-Fc | KFNWYVDGVEVHNAKTKPREEQYASTYRVVSVLTVLHQDWLNGKEYKCK |
| | region (CH2- | VSNKALPAPIEKTISKAKGQPREPQVCTLPPSREEMTKNQVSLSCAVKGFYPS |
| | CH3) Hole | DIAVEWESNGQPENNYKTTPPVLDSDGSFFLVSKLTVDKSRWQQGNVFSCSV |
| | | MHEALHNHYTQKSLSLSPGK |
| SEQ ID | Heavy chain | DKTHTCPPCPAPELLGGPSVFLFPPKPKDTLMISRTPEVTCVVVDVSHEDPEV |
| NO: 511 | hinge-Fc | KFNWYVDGVEVHNAKTKPREEQYASTYRVVSVLTVLHQDWLNGKEYKCK |
| | region (CH2- | VSNKALPAPIEKTISKAKGQPREPQVCTLPPSREEMTKNQVSLSCAVKGFYPS |
| | CH3) Hole | DIAVEWESNGQPENNYKTTPPVLDSDGSFFLVSKLTVDKSRWQQGNVFSCSV |
| | | MHEALHNHYTQKSLSLSP |
| SEQ ID | TRBC 2 | PRGFYGYHMH |
| NO: 8272 | VHCDR1 | |
| SEQ ID | TRBC 2 | FINPYNNHIQYNERFRG |
| NO: 8044 | VHCDR2 | |
| SEQ ID | TRBC 2 | GNGKWGDGAYRFFDF |
| NO: 7403 | VHCDR3 | |
| SEQ ID | TRBC 2 | RSSKNLVHSNGRTYLQ |
| NO: 8051 | VLCDR1 | |
| SEQ ID | TRBC 2 | RVSNRFP |
| NO: 224 | VLCDR2 | |
| SEQ ID | TRBC 2 | SQSTREPYT |
| NO: 8052 | VLCDR3 | |
| SEQ ID | TRBC2 VH | QVQLVQSGAEVKKPGSSVKVSCKASPRGFYGYHMHWVRQAPGQGLEWMG |
| NO: 8013 | | FINPYNNHIQYNERFRGRVTITSDESTTTAYMELSSLRSEDTAVYYCALGNGK |
| | | WGDGAYRFFDFWGQGTLVTVSS |
| SEQ ID | TRBC2 VL | DVVMTQSPLSLPVTLGQPASISCRSSKNLVHSNGRTYLQWYQQRPGQSPRLLI |
| NO: 8014 | | YRVSNRFPGVPDRFSGSGSGTDFTLKISRVEAEDVGVYFCSQSTREPYTFGGG |
| | | TKVEIK |
| SEQ ID | Light chain | RTVAAPSVFIFPPSDEQLKSGTASVVCLLNNFYPREAKVQWKVDNALQSGNS |
| NO: 3644 | constant | QESVTEQDSKDSTYSLSSTLTLSKADYEKHKVYACEVTHQGLSSPVTKSFNR |
| | region | GEC |
| SEQ ID | Heavy chain | ASTKGPSVFPLAPSSKSTSGGTAALGCLVKDYFPEPVTVSWNSGALTSGVHTF |
| NO: 513 | constant | PAVLQSSGLYSLSSVVTVPSSSLGTQTYICNVNHKPSNTKVDKRVEPKSCDKT |
| | region (CH1- | HTCPPCPAPELLGGPSVFLFPPKPKDTLMISRTPEVTCVVVDVSHEDPEVKFN |
| | CH2-CH3) | WYVDGVEVHNAKTKPREEQYASTYRVVSVLTVLHQDWLNGKEYKCKVSN |
| | Knob | KALPAPIEKTISKAKGQPREPQVYTLPPCREEMTKNQVSLWCLVKGFYPSDIA |
| | | VEWESNGQPENNYKTTPPVLDSDGSFFLYSKLTVDKSRWQQGNVFSCSVMH |
| | | EALHNHYTQKSLSLSPGK |
| SEQ ID | Heavy chain | ASTKGPSVFPLAPSSKSTSGGTAALGCLVKDYFPEPVTVSWNSGALTSGVHTF |
| NO: 514 | constant | PAVLQSSGLYSLSSVVTVPSSSLGTQTYICNVNHKPSNTKVDKRVEPKSCDKT |
| | region (CH1- | HTCPPCPAPELLGGPSVFLFPPKPKDTLMISRTPEVTCVVVDVSHEDPEVKFN |
| | CH2-CH3) | WYVDGVEVHNAKTKPREEQYASTYRVVSVLTVLHQDWLNGKEYKCKVSN |
| | Knob | KALPAPIEKTISKAKGQPREPQVYTLPPCREEMTKNQVSLWCLVKGFYPSDIA |
| | | VEWESNGQPENNYKTTPPVLDSDGSFFLYSKLTVDKSRWQQGNVFSCSVMH |
| | | EALHNHYTQKSLSLSP |
| SEQ ID | Heavy chain | EPKSCDKTHTCPPCPAPELLGGPSVFLFPPKPKDTLMISRTPEVTCVVVDVSHE |
| NO: 515 | hinge-Fc | DPEVKFNWYVDGVEVHNAKTKPREEQYASTYRVVSVLTVLHQDWLNGKEY |
| | region (CH2- | KCKVSNKALPAPIEKTISKAKGQPREPQVYTLPPCREEMTKNQVSLWCLVKG |
| | CH3) Knob | FYPSDIAVEWESNGQPENNYKTTPPVLDSDGSFFLYSKLTVDKSRWQQGNVF |
| | | SCSVMHEALHNHYTQKSLSLSPGK |
| SEQ ID | Heavy chain | EPKSCDKTHTCPPCPAPELLGGPSVFLFPPKPKDTLMISRTPEVTCVVVDVSHE |
| NO: 516 | hinge-Fc | DPEVKFNWYVDGVEVHNAKTKPREEQYASTYRVVSVLTVLHQDWLNGKEY |
| | region (CH2- | KCKVSNKALPAPIEKTISKAKGQPREPQVYTLPPCREEMTKNQVSLWCLVKG |
| | CH3) Knob | FYPSDIAVEWESNGQPENNYKTTPPVLDSDGSFFLYSKLTVDKSRWQQGNVF |
| | | SCSVMHEALHNHYTQKSLSLSP |

Exemplary molecule 11 Anti-TRBC2-NKp30

| | | |
|---|---|---|
| SEQ ID | NKp30 scFv- | EIQLLESGGGLVQPGGSLRLSCAVSGFSIIRVGYHWNWVRQAPGKGLEWVGY |
| NO: 509 | CH-Hole | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | ATLKISRVEAEDVGVYFCQFWDSTNSAVFGGGTKVEIKDKTHTCPPCPAPELL |
| | | GGPSVFLFPPKPKDTLMISRTPEVTCVVVDVSHEDPEVKFNWYVDGVEVHNA |
| | | KTKPREEQYASTYRVVSVLTVLHQDWLNGKEYKCKVSNKALPAPIEKTISKA |
| | | KGQPREPQVCTLPPSREEMTKNQVSLSCAVKGFYPSDIAVEWESNGQPENNY |
| | | KTTPPVLDSDGSFFLVSKLTVDKSRWQQGNVFSCSVMHEALHNHYTQKSLSL |
| | | SPGK |
| SEQ ID | NKp30 scFv- | EIQLLESGGGLVQPGGSLRLSCAVSGFSIIRVGYHWNWVRQAPGKGLEWVGY |
| NO: 4704 | CH-Hole | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | ATLKISRVEAEDVGVYFCQFWDSTNSAVFGGGTKVEIKDKTHTCPPCPAPELL |
| | | GGPSVFLFPPKPKDTLMISRTPEVTCVVVDVSHEDPEVKFNWYVDGVEVHNA |
| | | KTKPREEQYASTYRVVSVLTVLHQDWLNGKEYKCKVSNKALPAPIEKTISKA |
| | | KGQPREPQVCTLPPSREEMTKNQVSLSCAVKGFYPSDIAVEWESNGQPENNY |
| | | KTTPPVLDSDGSFFLVSKLTVDKSRWQQGNVFSCSVMHEALHNHYTQKSLSL |
| | | SP |
| SEQ ID | Light chain | DVVMTQSPLSLPVTLGQPASISCRSSKNLVHSNGRTYLQWYQQRPGQSPRLLI |
| NO: 8005 | (VL-CL) | YRVSNRFPGVPDRFSGSGSGTDFTLKISRVEAEDVGVYFCSQSTREPYTFGGG |
| | | TKVEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSPVT |
| | | KSFNRGEC |
| SEQ ID | Heavy chain | QVQLVQSGAEVKKPGSSVKVSCKASPRGFYGYHMHWVRQAPGQGLEWMG |
| NO: 8007 | (TRBC2 VH- | FINPYNNHIQYNERFRGRVTITSDESTTTAYMELSSLRSEDTAVYYCALGEGK |
| | CH)-knob | WGDGAYRFFDFWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYASTYRVVS |
| | | VLTVLHQDWLNGKEYKCKVSNKALPAPIEKTISKAKGQPREPQVYTLPPCRE |
| | | EMTKNQVSLWCLVKGFYPSDIAVEWESNGQPENNYKTTPPVLDSDGSFFLYS |
| | | KLTVDKSRWQQGNVFSCSVMHEALHNHYTQKSLSLSPGK |
| SEQ ID | Heavy chain | QVQLVQSGAEVKKPGSSVKVSCKASPRGFYGYHMHWVRQAPGQGLEWMG |
| NO: 4686 | (TRBC2 VH- | FINPYNNHIQYNERFRGRVTITSDESTTTAYMELSSLRSEDTAVYYCALGEGK |
| | CH)-knob | WGDGAYRFFDFWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYASTYRVVS |
| | | VLTVLHQDWLNGKEYKCKVSNKALPAPIEKTISKAKGQPREPQVYTLPPCRE |
| | | EMTKNQVSLWCLVKGFYPSDIAVEWESNGQPENNYKTTPPVLDSDGSFFLYS |
| | | KLTVDKSRWQQGNVFSCSVMHEALHNHYTQKSLSLSP |
| SEQ ID | NKp30 | GFSIIRVGYHWN |
| NO: 502 | VHCDR1 | |
| SEQ ID | NKp30 | YIHRSGSTSYNPSLKS |
| NO: 503 | VHCDR2 | |
| SEQ ID | NKp30 | GDWHYFDY |
| NO: 504 | VHCDR3 | |
| SEQ ID | NKp30 | SGEKLSDKYVH |
| NO: 26 | VLCDR1 | |
| SEQ ID | NKp30 | ENDRRPS |
| NO: 28 | VLCDR2 | |
| SEQ ID | NKp30 | QFWDSTNSAV |
| NO: 86 | VLCDR3 | |
| SEQ ID | NKp30 VH | EIQLLESGGGLVQPGGSLRLSCAVSGFSIIRVGYHWNWVRQAPGKGLEWVGY |
| NO: 505 | (BM130) | IHRSGSTSYNPSLKSRFTISRDTSKNTFYLQMNSLRAEDTAVYYCARGDWHYF |
| | | DYWGQGTMVTVSS |
| SEQ ID | NKp30 VL | DSVTTQSPLSLPVTLGQPASISCSGEKLSDKYVHWYQQRPGQSPRMLIYENDR |
| NO: 13 | (BM130) | RPSGVPDRFSGSNSGNDATLKISRVEAEDVGVYFCQFWDSTNSAVFGGGTKV |
| | | EIK |
| SEQ ID | Linker | GGGGSGGGGSGGGGSGGGGS |
| NO: 8291 | | |
| SEQ ID | Nkp30 scFv | EIQLLESGGGLVQPGGSLRLSCAVSGFSIIRVGYHWNWVRQAPGKGLEWVGY |
| NO: 518 | | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | ATLKISRVEAEDVGVYFCQFWDSTNSAVFGGGTKVEIK |
| SEQ ID | Heavy chain | DKTHTCPPCPAPELLGGPSVFLFPPKPKDTLMISRTPEVTCVVVDVSHEDPEV |
| NO: 3447 | hinge-Fc | KFNWYVDGVEVHNAKTKPREEQYASTYRVVSVLTVLHQDWLNGKEYKCK |
| | region (CH2- | VSNKALPAPIEKTISKAKGQPREPQVCTLPPSREEMTKNQVSLSCAVKGFYPS |
| | CH3) Hole | DIAVEWESNGQPENNYKTTPPVLDSDGSFFLVSKLTVDKSRWQQGNVFSCSV |
| | | MHEALHNHYTQKSLSLSPGK |
| SEQ ID | Heavy chain | DKTHTCPPCPAPELLGGPSVFLFPPKPKDTLMISRTPEVTCVVVDVSHEDPEV |
| NO: 511 | hinge-Fc | KFNWYVDGVEVHNAKTKPREEQYASTYRVVSVLTVLHQDWLNGKEYKCK |
| | region (CH2- | VSNKALPAPIEKTISKAKGQPREPQVCTLPPSREEMTKNQVSLSCAVKGFYPS |
| | CH3) Hole | DIAVEWESNGQPENNYKTTPPVLDSDGSFFLVSKLTVDKSRWQQGNVFSCSV |
| | | MHEALHNHYTQKSLSLSP |
| SEQ ID | TRBC 2 | PRGFYGYHMH |
| NO: 8272 | VHCDR1 | |
| SEQ ID | TRBC 2 | FINPYNNHIQYNERFRG |
| NO: 8044 | VHCDR2 | |
| SEQ ID | TRBC 2 | GEGKWGDGAYRFFDF |
| NO: 8046 | VHCDR3 | |
| SEQ ID | TRBC 2 | RSSKNLVHSNGRTYLQ |
| NO: 8051 | VLCDR1 | |
| SEQ ID | TRBC 2 | RVSNRFP |
| NO: 224 | VLCDR2 | |
| SEQ ID | TRBC 2 | SQSTREPYT |
| NO: 8052 | VLCDR3 | |
| SEQ ID | TRBC2 VH | QVQLVQSGAEVKKPGSSVKVSCKASPRGFYGYHMHWVRQAPGQGLEWMG |
| NO: 8299 | | FINPYNNHIQYNERFRGRVTITSDESTTTAYMELSSLRSEDTAVYYCALGEGK |
| | | WGDGAYRFFDFWGQGTLVTVSS |
| SEQ ID | TRBC2 VL | DVVMTQSPLSLPVTLGQPASISCRSSKNLVHSNGRTYLQWYQQRPGQSPRLLI |
| NO: 8014 | | YRVSNRFPGVPDRFSGSGSGTDFTLKISRVEAEDVGVYFCSQSTREPYTFGGG |
| | | TKVEIK |
| SEQ ID | Light chain | RTVAAPSVFIFPPSDEQLKSGTASVVCLLNNFYPREAKVQWKVDNALQSGNS |
| NO: 3644 | constant | QESVTEQDSKDSTYSLSSTLTLSKADYEKHKVYACEVTHQGLSSPVTKSFNR |
| | region | GEC |
| SEQ ID | Heavy chain | ASTKGPSVFPLAPSSKSTSGGTAALGCLVKDYFPEPVTVSWNSGALTSGVHTF |
| NO: 513 | constant | PAVLQSSGLYSLSSVVTVPSSSLGTQTYICNVNHKPSNTKVDKRVEPKSCDKT |
| | region (CH1- | HTCPPCPAPELLGGPSVFLFPPKPKDTLMISRTPEVTCVVVDVSHEDPEVKFN |
| | CH2-CH3) | WYVDGVEVHNAKTKPREEQYASTYRVVSVLTVLHQDWLNGKEYKCKVSN |
| | Knob | KALPAPIEKTISKAKGQPREPQVYTLPPCREEMTKNQVSLWCLVKGFYPSDIA |
| | | VEWESNGQPENNYKTTPPVLDSDGSFFLYSKLTVDKSRWQQGNVFSCSVMH |
| | | EALHNHYTQKSLSLSPGK |
| SEQ ID | Heavy chain | ASTKGPSVFPLAPSSKSTSGGTAALGCLVKDYFPEPVTVSWNSGALTSGVHTF |
| NO: 514 | constant | PAVLQSSGLYSLSSVVTVPSSSLGTQTYICNVNHKPSNTKVDKRVEPKSCDKT |
| | region (CH1- | HTCPPCPAPELLGGPSVFLFPPKPKDTLMISRTPEVTCVVVDVSHEDPEVKFN |
| | CH2-CH3) | WYVDGVEVHNAKTKPREEQYASTYRVVSVLTVLHQDWLNGKEYKCKVSN |
| | Knob | KALPAPIEKTISKAKGQPREPQVYTLPPCREEMTKNQVSLWCLVKGFYPSDIA |
| | | VEWESNGQPENNYKTTPPVLDSDGSFFLYSKLTVDKSRWQQGNVFSCSVMH |
| | | EALHNHYTQKSLSLSP |
| SEQ ID | Heavy chain | EPKSCDKTHTCPPCPAPELLGGPSVFLFPPKPKDTLMISRTPEVTCVVVDVSHE |
| NO: 515 | hinge-Fc | DPEVKFNWYVDGVEVHNAKTKPREEQYASTYRVVSVLTVLHQDWLNGKEY |
| | region (CH2- | KCKVSNKALPAPIEKTISKAKGQPREPQVYTLPPCREEMTKNQVSLWCLVKG |
| | CH3) Knob | FYPSDIAVEWESNGQPENNYKTTPPVLDSDGSFFLYSKLTVDKSRWQQGNVF |
| | | SCSVMHEALHNHYTQKSLSLSPGK |
| SEQ ID | Heavy chain | EPKSCDKTHTCPPCPAPELLGGPSVFLFPPKPKDTLMISRTPEVTCVVVDVSHE |
| NO: 516 | hinge-Fc | DPEVKFNWYVDGVEVHNAKTKPREEQYASTYRVVSVLTVLHQDWLNGKEY |
| | region (CH2- | KCKVSNKALPAPIEKTISKAKGQPREPQVYTLPPCREEMTKNQVSLWCLVKG |
| | CH3) Knob | FYPSDIAVEWESNGQPENNYKTTPPVLDSDGSFFLYSKLTVDKSRWQQGNVF |
| | | SCSVMHEALHNHYTQKSLSLSP |

Exemplary molecule 12 Anti-TRBC2-NKp30

| | | |
|---|---|---|
| SEQ ID | NKp30 scFv- | EIQLLESGGGLVQPGGSLRLSCAVSGFSIIRVGYHWNWVRQAPGKGLEWVGY |
| NO: 509 | CH-Hole | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | ATLKISRVEAEDVGVYFCQFWDSTNSAVFGGGTKVEIKDKTHTCPPCPAPELL |
| | | GGPSVFLFPPKPKDTLMISRTPEVTCVVVDVSHEDPEVKFNWYVDGVEVHNA |
| | | KTKPREEQYASTYRVVSVLTVLHQDWLNGKEYKCKVSNKALPAPIEKTISKA |
| | | KGQPREPQVCTLPPSREEMTKNQVSLSCAVKGFYPSDIAVEWESNGQPENNY |
| | | KTTPPVLDSDGSFFLVSKLTVDKSRWQQGNVFSCSVMHEALHNHYTQKSLSL |
| | | SPGK |
| SEQ ID | NKp30 scFv- | EIQLLESGGGLVQPGGSLRLSCAVSGFSIIRVGYHWNWVRQAPGKGLEWVGY |
| NO: 4704 | CH-Hole | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | ATLKISRVEAEDVGVYFCQFWDSTNSAVFGGGTKVEIKDKTHTCPPCPAPELL |
| | | GGPSVFLFPPKPKDTLMISRTPEVTCVVVDVSHEDPEVKFNWYVDGVEVHNA |
| | | KTKPREEQYASTYRVVSVLTVLHQDWLNGKEYKCKVSNKALPAPIEKTISKA |
| | | KGQPREPQVCTLPPSREEMTKNQVSLSCAVKGFYPSDIAVEWESNGQPENNY |
| | | KTTPPVLDSDGSFFLVSKLTVDKSRWQQGNVFSCSVMHEALHNHYTQKSLSL |
| | | SP |
| SEQ ID | Light chain | DVVMTQSPLSLPVTLGQPASISCRSSKNLVHSNGRTYLQWYQQRPGQSPRLLI |
| NO: 8005 | (VL-CL) | YRVSNRFPGVPDRFSGSGSGTDFTLKISRVEAEDVGVYFCSQSTREPYTFGGG |
| | | TKVEIKRTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQGLSSPVT |
| | | KSFNRGEC |
| SEQ ID | Heavy chain | QVQLVQSGAEVKKPGSSVKVSCKASPRGFYGYHMHWVRQAPGQGLEWMG |
| NO: 8009 | (TRBC2 VH- | FINPYNNHIQYNERFRGRVTITSDESTTTAYMELSSLRSEDTAVYYCALGAGK |
| | CH)-knob | WGDGAYRFFDFWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYASTYRVVS |
| | | VLTVLHQDWLNGKEYKCKVSNKALPAPIEKTISKAKGQPREPQVYTLPPCRE |
| | | EMTKNQVSLWCLVKGFYPSDIAVEWESNGQPENNYKTTPPVLDSDGSFFLYS |
| | | KLTVDKSRWQQGNVFSCSVMHEALHNHYTQKSLSLSPGK |
| SEQ ID | Heavy chain | QVQLVQSGAEVKKPGSSVKVSCKASPRGFYGYHMHWVRQAPGQGLEWMG |
| NO: 4687 | (TRBC2 VH- | FINPYNNHIQYNERFRGRVTITSDESTTTAYMELSSLRSEDTAVYYCALGAGK |
| | CH)-knob | WGDGAYRFFDFWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKPKDTLMI |
| | | SRTPEVTCVVVDVSHEDPEVKFNWYVDGVEVHNAKTKPREEQYASTYRVVS |
| | | VLTVLHQDWLNGKEYKCKVSNKALPAPIEKTISKAKGQPREPQVYTLPPCRE |
| | | EMTKNQVSLWCLVKGFYPSDIAVEWESNGQPENNYKTTPPVLDSDGSFFLYS |
| | | KLTVDKSRWQQGNVFSCSVMHEALHNHYTQKSLSLSP |
| SEQ ID | NKp30 | GFSIIRVGYHWN |
| NO: 502 | VHCDR1 | |
| SEQ ID | NKp30 | YIHRSGSTSYNPSLKS |
| NO: 503 | VHCDR2 | |
| SEQ ID | NKp30 | GDWHYFDY |
| NO: 504 | VHCDR3 | |
| SEQ ID | NKp30 | SGEKLSDKYVH |
| NO: 26 | VLCDR1 | |
| SEQ ID | NKp30 | ENDRRPS |
| NO: 28 | VLCDR2 | |
| SEQ ID | NKp30 | QFWDSTNSAV |
| NO: 86 | VLCDR3 | |
| SEQ ID | NKp30 VH | EIQLLESGGGLVQPGGSLRLSCAVSGFSIIRVGYHWNWVRQAPGKGLEWVGY |
| NO: 505 | (BM130) | IHRSGSTSYNPSLKSRFTISRDTSKNTFYLQMNSLRAEDTAVYYCARGDWHYF |
| | | DYWGQGTMVTVSS |
| SEQ ID | NKp30 VL | DSVTTQSPLSLPVTLGQPASISCSGEKLSDKYVHWYQQRPGQSPRMLIYENDR |
| NO: 13 | (BM130) | RPSGVPDRFSGSNSGNDATLKISRVEAEDVGVYFCQFWDSTNSAVFGGGTKV |
| | | EIK |
| SEQ ID | Linker | GGGGSGGGGSGGGGSGGGGS |
| NO: 8291 | | |
| SEQ ID | Nkp30 scFv | EIQLLESGGGLVQPGGSLRLSCAVSGFSIIRVGYHWNWVRQAPGKGLEWVGY |
| NO: 518 | | IHRSGSTSYNPSLKSRFTISRDTSKNTFYLQMNSLRAEDTAVYYCARGDWHYF |
| | | DYWGQGTMVTVSSGGGGSGGGGSGGGGSGGGGSDSVTTQSPLSLPVTLGQP |
| | | ASISCSGEKLSDKYVHWYQQRPGQSPRMLIYENDRRPSGVPDRFSGSNSGND |
| | | ATLKISRVEAEDVGVYFCQFWDSTNSAVFGGGTKVEIK |
| SEQ ID | Heavy chain | DKTHTCPPCPAPELLGGPSVFLFPPKPKDTLMISRTPEVTCVVVDVSHEDPEV |
| NO: 3447 | hinge-Fc | KFNWYVDGVEVHNAKTKPREEQYASTYRVVSVLTVLHQDWLNGKEYKCK |
| | region (CH2- | VSNKALPAPIEKTISKAKGQPREPQVCTLPPSREEMTKNQVSLSCAVKGFYPS |
| | CH3) Hole | DIAVEWESNGQPENNYKTTPPVLDSDGSFFLVSKLTVDKSRWQQGNVFSCSV |
| | | MHEALHNHYTQKSLSLSPGK |
| SEQ ID | Heavy chain | DKTHTCPPCPAPELLGGPSVFLFPPKPKDTLMISRTPEVTCVVVDVSHEDPEV |
| NO: 511 | hinge-Fc | KFNWYVDGVEVHNAKTKPREEQYASTYRVVSVLTVLHQDWLNGKEYKCK |
| | region (CH2- | VSNKALPAPIEKTISKAKGQPREPQVCTLPPSREEMTKNQVSLSCAVKGFYPS |
| | CH3) Hole | DIAVEWESNGQPENNYKTTPPVLDSDGSFFLVSKLTVDKSRWQQGNVFSCSV |
| | | MHEALHNHYTQKSLSLSP |
| SEQ ID | TRBC 2 | PRGFYGYHMH |
| NO: 8272 | VHCDR1 | |
| SEQ ID | TRBC 2 | FINPYNNHIQYNERFRG |
| NO: 8044 | VHCDR2 | |
| SEQ ID | TRBC 2 | GAGKWGDGAYRFFDF |
| NO: 8047 | VHCDR3 | |
| SEQ ID | TRBC 2 | RSSKNLVHSNGRTYLQ |
| NO: 8051 | VLCDR1 | |
| SEQ ID | TRBC 2 | RVSNRFP |
| NO: 224 | VLCDR2 | |
| SEQ ID | TRBC 2 | SQSTREPYT |
| NO: 8052 | VLCDR3 | |
| SEQ ID | TRBC2 VH | QVQLVQSGAEVKKPGSSVKVSCKASPRGFYGYHMHWVRQAPGQGLEWMG |
| NO: 8303 | | FINPYNNHIQYNERFRGRVTITSDESTTTAYMELSSLRSEDTAVYYCALGAGK |
| | | WGDGAYRFFDFWGQGTLVTVSS |
| SEQ ID | TRBC2 VL | DVVMTQSPLSLPVTLGQPASISCRSSKNLVHSNGRTYLQWYQQRPGQSPRLLI |
| NO: 8014 | | YRVSNRFPGVPDRFSGSGSGTDFTLKISRVEAEDVGVYFCSQSTREPYTFGGG |
| | | TKVEIK |
| SEQ ID | Light chain | RTVAAPSVFIFPPSDEQLKSGTASVVCLLNNFYPREAKVQWKVDNALQSGNS |
| NO: 3644 | constant | QESVTEQDSKDSTYSLSSTLTLSKADYEKHKVYACEVTHQGLSSPVTKSFNR |
| | region | GEC |
| SEQ ID | Heavy chain | ASTKGPSVFPLAPSSKSTSGGTAALGCLVKDYFPEPVTVSWNSGALTSGVHTF |
| NO: 513 | constant | PAVLQSSGLYSLSSVVTVPSSSLGTQTYICNVNHKPSNTKVDKRVEPKSCDKT |
| | region (CH1- | HTCPPCPAPELLGGPSVFLFPPKPKDTLMISRTPEVTCVVVDVSHEDPEVKFN |
| | CH2-CH3) | WYVDGVEVHNAKTKPREEQYASTYRVVSVLTVLHQDWLNGKEYKCKVSN |
| | Knob | KALPAPIEKTISKAKGQPREPQVYTLPPCREEMTKNQVSLWCLVKGFYPSDIA |
| | | VEWESNGQPENNYKTTPPVLDSDGSFFLYSKLTVDKSRWQQGNVFSCSVMH |
| | | EALHNHYTQKSLSLSPGK |
| SEQ ID | Heavy chain | ASTKGPSVFPLAPSSKSTSGGTAALGCLVKDYFPEPVTVSWNSGALTSGVHTF |
| NO: 514 | constant | PAVLQSSGLYSLSSVVTVPSSSLGTQTYICNVNHKPSNTKVDKRVEPKSCDKT |
| | region (CH1- | HTCPPCPAPELLGGPSVFLFPPKPKDTLMISRTPEVTCVVVDVSHEDPEVKFN |
| | CH2-CH3) | WYVDGVEVHNAKTKPREEQYASTYRVVSVLTVLHQDWLNGKEYKCKVSN |
| | Knob | KALPAPIEKTISKAKGQPREPQVYTLPPCREEMTKNQVSLWCLVKGFYPSDIA |
| | | VEWESNGQPENNYKTTPPVLDSDGSFFLYSKLTVDKSRWQQGNVFSCSVMH |
| | | EALHNHYTQKSLSLSP |
| SEQ ID | Heavy chain | EPKSCDKTHTCPPCPAPELLGGPSVFLFPPKPKDTLMISRTPEVTCVVVDVSHE |
| NO: 515 | hinge-Fc | DPEVKFNWYVDGVEVHNAKTKPREEQYASTYRVVSVLTVLHQDWLNGKEY |
| | region (CH2- | KCKVSNKALPAPIEKTISKAKGQPREPQVYTLPPCREEMTKNQVSLWCLVKG |
| | CH3) Knob | FYPSDIAVEWESNGQPENNYKTTPPVLDSDGSFFLYSKLTVDKSRWQQGNVF |
| | | SCSVMHEALHNHYTQKSLSLSPGK |
| SEQ ID | Heavy chain | EPKSCDKTHTCPPCPAPELLGGPSVFLFPPKPKDTLMISRTPEVTCVVVDVSHE |
| NO: 516 | hinge-Fc | DPEVKFNWYVDGVEVHNAKTKPREEQYASTYRVVSVLTVLHQDWLNGKEY |
| | region (CH2- | KCKVSNKALPAPIEKTISKAKGQPREPQVYTLPPCREEMTKNQVSLWCLVKG |
| | CH3) Knob | FYPSDIAVEWESNGQPENNYKTTPPVLDSDGSFFLYSKLTVDKSRWQQGNVF |
| | | SCSVMHEALHNHYTQKSLSLSP |

TABLE 42A
Exemplary anti-TCRβV antibody molecules

| TRBV | TRBV | Reagents monoclonal antibodies | | |
|---|---|---|---|---|
| gene name | allele name | Clone name and Specificity | Company product | Isotype |
| TRBV2 | TRBV2*01 | IsMMU 546 (TRBV2) | Serotec V BETA 22 | Mouse IgG1 |
| | TRBV2*02 | | Coulter Vbeta22 | |
| | TRBV2*03 | | | |
| TRBV3-1 | TRBV3-1*01 | FIN9 (TRBV3-1) | Serotec Vbeta9 | Mouse IgG2a |
| | | | Coulter Vbeta9 | |
| | TRBV3-1*02 | AMKB1-2 (TRBV3-1) | BD Biosciences Vbeta9 | Mouse IgG1 |
| TRBV4-1 | TRBV4-1*01 | ZOE (TRBV4-1, TRBV4-2, | Serotec V BETA 7 | Mouse IgG2a |
| | | TRBV4-3) | Coulter Vbeta7 | |
| | TRBV4-1*02 | 3G5 (TRBV4-1) | Pierce Endogen V beta 7.1 | Mouse IgG2b |
| TRBV4-2 | TRBV4-2*01 | ZOE (TRBV4-1, TRBV4-2, | Serotec V BETA 7 | Mouse IgG2a |
| | TRBV4-2*02 | TRBV4-3) | Coulter Vbeta7 | |
| TRBV4-3 | TRBV4-3*01 | ZOE (TRBV4-1, TRBV4-2, | Serotec V BETA 7 | Mouse IgG2a |
| | TRBV4-3*02 | TRBV4-3) | Coulter Vbeta7 | |
| | TRBV4-3*03 | | | |
| | TRBV4-3*04 | ZIZOU4 (TRBV4-3) | Coulter Vbeta7.2 | Mouse IgG2a |
| TRBV5-1 | TRBV5-1*01 | IMMU157 (TRBV5-1) | Serotec Vbeta5.1 | Mouse IgG2a |
| | | | Coulter Vbeta5.1 | |
| | TRBV5-1*02 | LC4(TRBV5-1) | Pierce Endogen V beta 5(c) | Mouse IgG1 |
| | | | BD Biosciences Vbeta5(c) | |
| TRBV5-4 | TRBV5-4*01 | | | |
| | TRBV5-4*02 | | | |
| | TRBV5-4*03 | | | |
| | TRBV5-4*04 | | | |
| TRBV5-5 | TRBV5-5*01 | 3D11 (TRBV5-5) | Serotec VBETA5.3 | Mouse IgG1 |
| | | | Coulter Vbeta5.3 | |
| | TRBV5-5*02 | 1C1 (TRBV5-5, TRBV5-6) | Pierce Endogen V beta 5(a) | Mouse IgG1 |
| | | | BD Biosciences Vbeta5(a) | |
| | TRBV5-5*03 | W112(TRBV5-5) | Pierce Endogen V beta 5(b) | Mouse IgG1 |
| | | | Serotec V beta 5.2/5.3 | |
| | | | BD Biosciences Vbeta5(b) | |
| | | MH3-2 (TRBV5-5, | BD Biosciences Vbeta5 | |
| | | TRBV5-6) | | Mouse IgG2a |
| | | 4H11 (TM27) as disclosed | | |
| | | in U.S. Pat. No. 5,861,155 | | |
| TRBV5-6 | TRBV5-6*01 | 36213 (TRBV5-6) | Serotec Vbeta5.2 | Mouse IgG1 |
| | | 1C1 (TRBV5-5, TRBV5-6) | BD Biosciences Vbeta5(a) | Mouse IgG1 |
| | | MH3-2 (TRBV5-5, | BD Biosciences Vbeta5 | Mouse IgG2a |
| | | TRBV5-6) | | |
| TRBV5-8 | TRBV5-8*01 | | | |
| | TRBV5-8*02 | | | |
| TRBV6-1 | TRBV6-1*01 | BAM13 (TRBV6-1, | Pierce Endogen V beta 13 | Mouse IgG1 |
| | | TRBV6-5) | BD Biosciences Vbeta13.1, | |
| | | | 13.3 | |
| TRBV6-2 | TRBV6-2*01 | H132 | Coulter Vbeta13.2 | Mouse IgG1 |
| TRBV6-3 | TRBV6-3*01 | | | |
| TRBV6-4 | TRBV6-4*01 | | | |
| | TRBV6-4*02 | | | |
| TRBV6-5 | TRBV6-5*01 | IMMU 222 (TRBV6-5, | Serotec V BETA 13.1 | Mouse IgG2b |
| | | TRBV6-6 and TRBV6-9) | Coulter Vbeta13.1 | |
| | | BAM13 (TRBV6-1, | Pierce Endogen V beta 13 | Mouse IgG1 |
| | | TRBV6-5) | BD Biosciences Vbeta13.1, | |
| | | | 13.3 | |
| TRBV6-6 | TRBV6-6*01 | JU-74 (TRBV6-6) | Serotec Vbeta13.6 | Mouse IgG1 |
| | TRBV6-6*02 | JU74.3 (TRBV6-6) | Coulter Vbeta13.6 | |
| | TRBV6-6*03 | | | |
| | TRBV6-6*04 | IMMU 222 (TRBV6-5, | Serotec V BETA 13.1 | Mouse IgG2b |
| | TRBV6-6*05 | TRBV6-6 and TRBV6-9) | Coulter Vbeta13.1 | |
| TRBV6-8 | TRBV6-8*01 | | | |
| TRBV6-9 | TRBV6-9*01 | IMMU 222 (TRBV6-5, | Serotec V BETA 13.1 | Mouse IgG2b |
| | | TRBV6-6 and TRBV6-9) | Coulter Vbeta13.1 | |
| TRBV7-2 | TRBV7-2*01 | OT145 (TRBV7-2) | Pierce Endogen V beta 6.7 | Mouse IgG1 |
| | TRBV7-2*02 | | BD Biosciences Vbeta6.7 | |
| | TRBV7-2*03 | | | |
| | TRBV7-2*04 | | | |
| TRBV7-3 | TRBV7-3*01 | | | |
| | TRBV7-3*04 | | | |
| | TRBV7-3*05 | | | |
| TRBV7-4 | TRBV7-4*01 | | | |
| TRBV7-6 | TRBV7-6*01 | | | |
| | TRBV7-6*02 | | | |
| TRBV7-7 | TRBV7-7*01 | | | |
| | TRBV7-7*02 | | | |
| TRBV7-8 | TRBV7-8*01 | | | |
| | TRBV7-8*02 | | | |
| | TRBV7-8*03 | | | |
| TRBV7-9 | TRBV7-9*01 | | | |
| | TRBV7-9*02 | | | |
| | TRBV7-9*03 | | | |
| | TRVB7-9*04 | | | |
| | TRBV7-9*05 | | | |
| | TRBV7-9*06 | | | |
| | TRBV7-9*07 | | | |
| TRBV9 | TRBV9*01 | BL37.2 (TRBV9) | Serotec Vbetal | Rat IgG1 |
| | TRBV9*02 | | Coulter Vbetal | |
| | TRBV9*03 | | | |
| TRBV10-1 | TRBV10-1*01 | S511 (TRBV10-1, | Pierce Endogen V beta 12 | Mouse IgG2b |
| | TRBV10-1*02 | TRBV10-2, TRBV10-3) | BD Biosciences Vbeta12 | |
| TRBV10-2 | TRBV10-2*01 | | | |
| | TRBV10-2*02 | | | |
| TRBV10-3 | TRBV10-3*01 | VER2.32.1 (TRBV10-3) | Serotec Vbeta12 | Mouse IgG2a |
| | TRBV10-3*02 | S511 (TRBV10-1, | Coulter Vbeta12 | |
| | TRBV10-3*03 | TRBV10-2, TRBV10-3) | Pierce Endogen V beta 12 | Mouse IgG2b |
| | TRBV10-3*04 | | BD Biosciences Vbeta12 | |
| TRBV11-1 | TRBV11-1*01 | | | |
| TRBV11-2 | TRBV11-2*01 | IG125 (TRBV11-2) | Serotec Vbeta21.3 | Mouse IgG2a |
| | TRBV11-2*02 | | Coulter Vbeta21.3 | |
| | TRBV11-2*03 | | | |
| TRBV11-3 | TRBV11-3*01 | | | |
| | TRBV11-3*02 | | | |
| | TRBV11-3*03 | | | |
| | TRBV11-3*04 | | | |
| TRBV12-3 | TRBV12-3*01 | 56C5 (TRBV12-3, | Serotec Vbeta8.1/8.2 | Mouse IgG2a |
| | | TRBV12-4) | Coulter Vbeta8 | |
| TRBV12-4 | TRBV12-4*01 | | Pierce Endogen V beta 8(a) | Mouse IgG2b |
| | | | BD Biosciences Vbeta8 | |
| | TRBV12-4*02 | 56C5.2 (TRBV12-3, | Pierce Endogen V beta 8(b) | Mouse IgG2a |
| | | TRBV12-4) | | |
| | | 16G8 (TRBV12-3, | BD Biosciences | Mouse IgG2b |
| | | TRBV12-4) | Vbeta8 | |
| | | MX-6 (TRBV12-3, | | |
| | | TRBV12-4) | | |
| | | JR2 (TRBV12-3, TRBV12- | | |
| | | 4, TRBV12-5) | | |
| TRBV12-5 | TRBV12-5*01 | JR2 (TRBV12-3, TRBV12- | BD Biosciences Vbeta8 | Mouse IgG2b |
| | | 4, TRBV12-5) | | |
| TRBV13 | TRBV13*01 | AF-23 (TRBV13) | Serotec Vbeta23 | Mouse IgG1 |
| | TRBV13*02 | AF23 (TRBV13) | Coulter Vbeta23 | |
| | | AHUT7 (Vbeta23) | BD Biosciences Vbeta23 | |
| TRBV14 | TRBV14*01 | TAMAYA1.2 (TRBV14) | Serotec Vbeta16 | Mouse IgG1 |
| | TRBV14*02 | | Coulter Vbeta16 | |
| TRBV15 | TRBV15*01 | | | |
| | TRBV15*02 | | | |
| | TRBV15*03 | | | |
| TRBV16 | TRBV16*01 | | | |
| | TRBV16*03 | | | |
| TRBV18 | TRBV18*01 | BA62 (TRBV18) | Serotec V BETA 18 | Mouse IgG1 |
| | | BA62.6 (TRBV18) | Coulter Vbeta18 | |
| TRBV19 | TRBV19*01 | C1 (TRBV19) | Pierce Endogen V beta 17 | Mouse IgG1 |
| | | E17.5F3 (TRBV19) | BD Biosciences Vbeta17 | |
| | TRBV19*02 | E17.5F3.15.13 (TRBV19) | Serotec Vbeta17 | Mouse IgG1 |
| | TRBV19*03 | | Coulter Vbeta17 | |
| TRBV20-1 | TRBV20-1*01 | MPB2D5 (TRBV20-1) | Serotec VBETA2 | Mouse IgG1 |
| | TRBV20-1*02 | | Coulter Vbeta2 | |
| | TRBV20-1*03 | | | |
| | TRBV20-1*04 | | | |
| | TRBV20-1*05 | | | |
| | TRBV20-1*06 | | | |
| | TRBV20-1*07 | | | |
| TRBV24-1 | TRBV24-1*01 | | | |
| TRBV25-1 | TRBV25-1*01 | C21 (TRBV25-1) | Serotec V BETA 11 | Mouse IgG2a |
| | | | Coulter Vbeta11 | |
| TRBV27 | TRBV27*01 | CAS1.1.3 (TRBV27) | Serotec Vbeta14 | Mouse IgG1 |
| | | | Coulter Vbeta14 | |
| TRBV28 | TRBV28*01 | CH92 (TRBV28) | Serotec Vbeta3 | Mouse IgM |
| | | | Coulter Vbeta3 | |
| | | 8F10 (TRBV28) | Pierce Endogen V beta 3.1 | Mouse IgG1 |
| | | JOVI-3 (TRBV28) | BD Biosciences Vbeta3 | Mouse IgG2a |
| TRBV29-1 | TRBV29-1*01 | WJF24 | Coulter Vbeta4 | Rat IgM |
| | TRBV29-1*02 | | | |
| | TRBV29-1*03 | | | |
| TRBV30 | TRBV30*01 | ELL1.4 (TRBV30) | Serotec Vbeta20 | Mouse IgG1 |
| | TRBV30*02 | | Coulter Vbeta20 | |
| | TRBV30*04 | | | |
| | TRBV30*05 | | | |

TABLE 42B
Amino acid sequences for anti TCRβ V5 antibodies. Amino acid and nucleotide sequences for
murine and humanized antibody molecules which bind to TCRVB 5 (e.g., TCRVB 5-5 or TCRVB 5-6).
The amino acid of the heavy and light chain CDRs, and the amino acid and nucleotide sequences
of the heavy and light chain variable regions, and the heavy and light chains are shown.
Murine antibody C, also referred to as 4H11

| | | |
|---|---|---|
| SEQ ID NO: 1315 | HC CDR1 (Kabat) | AYGVN |
| SEQ ID NO: 1316 | HC CDR2 (Kabat) | MIWGDGNTDYNSALKS |
| SEQ ID NO: 1317 | HC CDR3 (Kabat) | DRVTATLYAMDY |
| SEQ ID NO: 1318 | HC CDR1 (Chothia) | GFSLTAY |
| SEQ ID NO: 1319 | HC CDR2 (Chothia) | WGDGN |
| SEQ ID NO: 1317 | HC CDR3 (Chothia) | DRVTATL YAMDY |
| SEQ ID NO: 1320 | HC CDR1 (Combined) | GFSLTAYGVN |
| SEQ ID NO: 1316 | HC CDR2 (Combined) | MIWGDGNTDYNSALKS |
| SEQ ID NO: 1317 | HC CDR3(Combined) | DRVTATLYAMDY |
| SEQ ID NO: 1321 | LC CDR1 (Kabat) | SASQGISNYLN |
| SEQ ID NO: 1322 | LC CDR2 (Kabat) | YTSSLHS |
| SEQ ID NO: 1323 | LC CDR3 (Kabat) | QQYSKLPRT |
| SEQ ID NO: 1321 | LC CDR1 (Chothia) | SASQGISNYLN |
| SEQ ID NO: 1322 | LC CDR2 (Chothia) | YTSSLHS |
| SEQ ID NO: 1323 | LC CDR3 (Chothia) | QQYSKLPRT |
| SEQ ID NO: 1321 | LC CDR1 (Combined) | SASQGISNYLN |
| SEQ ID NO: 1322 | LC CDR2 (Combined) | YTSSLHS |
| SEQ ID NO: 1323 | LC CDR3(Combined) | QQYSKLPRT |
| SEQ ID NO: 232 | VH | DIQMTQTTSSLSASLGDRVTISCSASQGISNYLNWYQQK |
| | | PDGTVKLLIYYTSSLHSGVPSRFSGSGSGTDYSLTISNLE |
| | | PEDIATYYCQQYSKLPRTFGGGTKVEIK |
| SEQ ID NO: 233 | VL | QVQLKESGPGLVAPSQSLSITCTVSGFSLTAYGVNWVR |
| | | QPPGKGLEWLGMIWGDGNTDYNSALKSRLSISKDNSKS |
| | | QVFLKMNSLQTDDTARYYCARDRVTATLYAMDYWGQ |
| | | GTSVTVSS |

Humanized antibody C C-H-1 antibody

| | | |
|---|---|---|
| SEQ ID NO: 1315 | HC CDR1 (Kabat) | AYGVN |
| SEQ ID NO: 1316 | HC CDR2 (Kabat) | MIWGDGNTDYNSALKS |
| SEQ ID NO: 1317 | HC CDR3 (Kabat) | DRVTATLYAMDY |
| SEQ ID NO: 1318 | HC CDR1 (Chothia) | GFSLTAY |
| SEQ ID NO: 1319 | HC CDR2 (Chothia) | WGDGN |
| SEQ ID NO: 1317 | HC CDR3 (Chothia) | DRVTATLYAMDY |
| SEQ ID NO: 1320 | HC CDR1 (Combined) | GFSLTAYGVN |
| SEQ ID NO: 1316 | HC CDR2 (Combined) | MIWGDGNTDYNSALKS |
| SEQ ID NO: 1317 | HC CDR3 (Combined) | DRVTATLYAMDY |
| SEQ ID NO: 1321 | LC CDR1 (Kabat) | SASQGISNYLN |
| SEQ ID NO: 1322 | LC CDR2 (Kabat) | YTSSLHS |
| SEQ ID NO: 1323 | LC CDR3 (Kabat) | QQYSKLPRT |
| SEQ ID NO: 1321 | LC CDR1 (Chothia) | SASQGISNYLN |
| SEQ ID NO: 1322 | LC CDR2 (Chothia) | YTSSLHS |
| SEQ ID NO: 1323 | LC CDR3 (Chothia) | QQYSKLPRT |
| SEQ ID NO: 1321 | LC CDR1 (Combined) | SASQGISNYLN |
| SEQ ID NO: 1322 | LC CDR2 (Combined) | YTSSLHS |
| SEQ ID NO: 1323 | LC CDR3(Combined) | QQYSKLPRT |
| SEQ ID NO: 1324 | VL | DIQMTQSPSSLSASVGDRVTITCSASQGISNYLNWYQQTP |
| | | GKAPKLLIYYTSSLHSGVPSRFSGSGSGTDYTFTISSLQPE |
| | | DIATYYCQQYSKLPRTFGQGTKLQIT |
| SEQ ID NO: 1325 | VH | QVQLQESGPGLVRPSQTLSLTCTVSGFSLTAYGVNWVR |
| | | QPPGRGLEWLGMIWGDGNTDYNSALKSRVTMLKDTSK |
| | | NQFSLRLSSVTAADTAVYYCARDRVTATLYAMDYW |
| | | GQGSLVTVSS |

Humanized antibody C Variable light chain (VL)

| | | | |
|---|---|---|---|
| SEQ ID NO: 3000 | VL | C-H-VL.1 | DIQMTQSPSFLSASVGDRVTITCSASQGISNYLNWYQQK |
| | | | PGKAVKLLIYYTSSLHSGVPSRFSGSGSGTEYTLTISSLQ |
| | | | PEDFATYYCQQYSKLPRTFGGGTKVEIK |
| SEQ ID NO: 3001 | VL | C-H-VL.2 | DIQMTQSPSSLSASVGDRVTITCSASQGISNYLNWYQQK |
| | | | PGKAVKLLIYYTSSLHSGVPSRFSGSGSGTDYTLTISSLQ |
| | | | PEDFATYYCQQYSKLPRTFGGGTKVEIK |
| SEQ ID NO: 3002 | VL | C-H-VL.3 | DIQMTQSPSSLSASVGDRVTITCSASQGISNYLNWYQQK |
| | | | PGKVVKLLIYYTSSLHSGVPSRFSGSGSGTDYTLTISSLQ |
| | | | PEDVATYYCQQYSKLPRTFGGGTKVEIK |
| SEQ ID NO: 3003 | VL | C-H-VL.4 | DIQMTQSPSSLSASVGDRVTITCSASQGISNYLNWYQQK |
| | | | PGQAVKLLIYYTSSLHSGVPSRFSGSGSGTDYTLTISSLQ |
| | | | PEDVATYYCQQYSKLPRTFGGGTKVEIK |
| SEQ ID NO: 3004 | VL | C-H-VL.5 | DIQMTQSPSSLSASVGDRVTITCSASQGISNYLNWYQQK |
| | | | PGKAVKLLIYYTSSLHSGVPSRFSGSGSGTDYTFTISSLQ |
| | | | PEDIATYYCQQYSKLPRTFGGGTKVEIK |
| SEQ ID NO: 3005 | VL | C-H-VL.6 | DIQMTQSPSSLSASVGDRVTITCSASQGISNYLNWYQQK |
| | | | PGKTVKLLIYYTSSLHSGIPSRFSGSGSGTDYTLTIRSLQP |
| | | | EDFATYYCQQYSKLPRTFGGGTKVEIK |
| SEQ ID NO: 3006 | VL | C-H-VL.7 | AIQMTQSPSSLSASVGDRVTITCSASQGISNYLNWYQQK |
| | | | PGKAVKLLIYYTSSLHSGVPSRFSGSGSGTDYTLTISSLQ |
| | | | PEDFATYYCQQYSKLPRTFGGGTKVEIK |
| SEQ ID NO: 3007 | VL | C-H-VL.8 | DIQMTQSPSSVSASVGDRVTITCSASQGISNYLNWYQQK |
| | | | PGKAVKLLIYYTSSLHSGVPSRFSGSGSGTDYTLTISSLQ |
| | | | PEDFATYYCQQYSKLPRTFGGGTKVEIK |
| SEQ ID NO: 3008 | VL | C-H-VL.9 | DIQMTQSPSSLSASVGDRVTITCSASQGISNYLNWYQQK |
| | | | PGKAVKRLIYYTSSLHSGVPSRFSGSGSGTEYTLTISNLQ |
| | | | PEDFATYYCQQYSKLPRTFGGGTKVEIK |
| SEQ ID NO: 3009 | VL | C-H-VL.10 | AIRMTQSPFSLSASVGDRVTITCSASQGISNYLNWYQQK |
| | | | PAKAVKLFIYYTSSLHSGVPSRFSGSGSGTDYTLTISSLQ |
| | | | PEDFATYYCQQYSKLPRTFGGGTKVEIK |
| SEQ ID NO: 3010 | VL | C-H-VL.11 | DIQMTQSPSSLSASVGDRVTITCSASQGISNYLNWYQQK |
| | | | PGKAVKRLIYYTSSLHSGVPSRFSGSGSGTEYTLTISSLQ |
| | | | PEDFATYYCQQYSKLPRTFGGGTKVEIK |
| SEQ ID NO: 3011 | VL | C-H-VL.12 | DIQMTQSPSTLSASVGDRVTITCSASQGISNYLNWYQQK |
| | | | PGKAVKLLIYYTSSLHSGVPSRFSGSGSGTEYTLTISSLQ |
| | | | PDDFATYYCQQYSKLPRTFGGGTKVEIK |
| SEQ ID NO: 3012 | VL | C-H-VL.13 | DIQMTQSPSSLSASVGDRVTITCSASQGISNYLNWYQQK |
| | | | PGKAVKSLIYYTSSLHSGVPSRFSGSGSGTDYTLTISSLQ |
| | | | PEDFATYYCQQYSKLPRTFGGGTKVEIK |
| SEQ ID NO: 3013 | VL | C-H-VL.14 | DIQMTQSPSSLSASVGDRVTITCSASQGISNYLNWYQQK |
| | | | PGKAVKSLIYYTSSLHSGVPSKFSGSGSGTDYTLTISSLQ |
| | | | PEDFATYYCQQYSKLPRTFGGGTKVEIK |
| SEQ ID NO: 3014 | VL | C-H-VL.15 | DIQMTQSPSSLSASVGDRVTITCSASQGISNYLNWYQQK |
| | | | PEKAVKSLIYYTSSLHSGVPSRFSGSGSGTDYTLTISSLQ |
| | | | PEDFATYYCQQYSKLPRTFGGGTKVEIK |
| SEQ ID NO: 3015 | VL | C-H-VL.16 | DIQMTQSPSAMSASVGDRVTITCSASQGISNYLNWYQQ |
| | | | KPGKVVKRLIYYTSSLHSGVPSRFSGSGSGTEYTLTISSL |
| | | | QPEDFATYYCQQYSKLPRTFGGGTKVEIK |
| SEQ ID NO: 3016 | VL | C-H-VL.17 | DIVMTQSPDSLAVSLGERATINCSASQGISNYLNWYQQ |
| | | | KPGQPVKLLIYYTSSLHSGVPDRFSGSGSGTDYTLTISSL |
| | | | QAEDVAVYYCQQYSKLPRTFGGGTKVEIK |
| SEQ ID NO: 3017 | VL | C-H-VL.18 | EIVMTQSPGTLSLSPGERATLSCSASQGISNYLNWYQQK |
| | | | PGQAVKLLIYYTSSLHSGIPDRFSGSGSGTDYTLTISRLEP |
| | | | EDFAVYYCQQYSKLPRTFGGGTKVEIK |
| SEQ ID NO: 3018 | VL | C-H-VL.19 | EIVMTQSPPTLSLSPGERVTLSCSASQGISNYLNWYQQK |
| | | | PGQAVKLLIYYTSSLHSGIPARFSGSGSGTDYTLTISSLQP |
| | | | EDFAVYYCQQYSKLPRTFGGGTKVEIK |
| SEQ ID NO: 3019 | VL | C-H-VL.20 | EIVMTQSPPTLSLSPGERVTLSCSASQGISNYLNWYQQK |
| | | | PGQAVKLLIYYTSSLHSSIPARFSGSGSGTDYTLTISSLQP |
| | | | EDFAVYYCQQYSKLPRTFGGGTKVEIK |
| SEQ ID NO: 3020 | VL | C-H-VL.21 | EIVMTQSPATLSLSPGERATLSCSASQGISNYLNWYQQK |
| | | | PGQAVKLLIYYTSSLHSGIPARFSGSGSGTDYTLTISSLEP |
| | | | EDFAVYYCQQYSKLPRTFGGGTKVEIK |
| SEQ ID NO: 3021 | VL | C-H-VL.22 | EIVMTQSPATLSLSPGERATLSCSASQGISNYLNWYQQK |
| | | | PGQAVKLLIYYTSSLHSGIPARFSGSGSGTDYTLTISRLEP |
| | | | EDFAVYYCQQYSKLPRTFGGGTKVEIK |
| SEQ ID NO: 3022 | VL | C-H-VL.23 | EIVMTQSPATLSLSPGERATLSCSASQGISNYLNWYQQK |
| | | | PGQAVKLLIYYTSSLHSGIPDRFSGSGSGTDYTLTISRLEP |
| | | | EDFAVYYCQQYSKLPRTFGGGTKVEIK |
| SEQ ID NO: 3023 | VL | C-H-VL.24 | EIVMTQSPATLSLSPGERATLSCSASQGISNYLNWYQQK |
| | | | PGLAVKLLIYYTSSLHSGIPDRFSGSGSGTDYTLTISRLEP |
| | | | EDFAVYYCQQYSKLPRTFGGGTKVEIK |
| SEQ ID NO: 3024 | VL | C-H-VL.25 | DIQMIQSPSFLSASVGDRVSIICSASQGISNYLNWYLQKP |
| | | | GKSVKLFIYYTSSLHSGVSSRFSGRGSGTDYTLTIISLKPE |
| | | | DFAAYYCQQYSKLPRTFGGGTKVEIK |
| SEQ ID NO: 3025 | VL | C-H-VL.26 | EIVMTQSPATLSLSPGERATLSCSASQGISNYLNWYQQK |
| | | | PGQAVKLLIYYTSSLHSGIPARFSGSGSGTDYTLTISSLQP |
| | | | EDFAVYYCQQYSKLPRTFGGGTKVEIK |
| SEQ ID NO: 3026 | VL | C-H-VL.27 | EIVMTQSPATLSLSPGERATLSCSASQGISNYLNWYQQK |
| | | | PGQAVKLLIYYTSSLHSGIPARFSGSGPGTDYTLTISSLEP |
| | | | EDFAVYYCQQYSKLPRTFGGGTKVEIK |
| SEQ ID NO: 3027 | VL | C-H-VL.28 | DIVMTQTPLSLSVTPGQPASISCSASQGISNYLNWYLQK |
| | | | PGQSVKLLIYYTSSLHSGVPDRFSGSGSGTDYTLKISRVE |
| | | | AEDVGVYYCQQYSKLPRTFGGGTKVEIK |
| SEQ ID NO: 3028 | VL | C-H-VL.29 | DIVMTQTPLSLSVTPGQPASISCSASQGISNYLNWYLQK |
| | | | PGQPVKLLIYYTSSLHSGVPDRFSGSGSGTDYTLKISRVE |
| | | | AEDVGVYYCQQYSKLPRTFGGGTKVEIK |
| SEQ ID NO: 3029 | VL | C-H-VL.30 | DIVMTQSPAFLSVTPGEKVTITCSASQGISNYLNWYQQK |
| | | | PDQAVKLLIYYTSSLHSGVPSRFSGSGSGTDYTFTISSLE |
| | | | AEDAATYYCQQYSKLPRTFGGGTKVEIK |
| SEQ ID NO: 3030 | VL | C-H-VL.31 | DIVMTQSPLSLPVTPGEPASISCSASQGISNYLNWYLQKP |
| | | | GQSVKLLIYYTSSLHSGVPDRFSGSGSGTDYTLKISRVE |
| | | | AEDVGVYYCQQYSKLPRTFGGGTKVEIK |
| SEQ ID NO: 3031 | VL | C-H-VL.32 | DIVMTQTPLSLPVTPGEPASISCSASQGISNYLNWYLQKP |
| | | | GQSVKLLIYYTSSLHSGVPDRFSGSGSGTDYTLKISRVE |
| | | | AEDVGVYYCQQYSKLPRTFGGGTKVEIK |
| SEQ ID NO: 3032 | VL | C-H-VL.33 | EIVMTQSPATLSVSPGERATLSCSASQGISNYLNWYQQK |
| | | | PGQAVKLLIYYTSSLHSGIPARFSGSGSGTEYTLTISILQS |
| | | | EDFAVYYCQQYSKLPRTFGGGTKVEIK |
| SEQ ID NO: 3033 | VL | C-H-VL.34 | EIVMTQSPATLSVSPGERATLSCSASQGISNYLNWYQQK |
| | | | PGQAVKLLIYYTSSLHSGIPARFSGSGSGTEYTLTISSLQS |
| | | | EDFAVYYCQQYSKLPRTFGGGTKVEIK |
| SEQ ID NO: 3034 | VL | C-H-VL.35 | DIVMTQSPLSLPVTLGQPASISCSASQGISNYLNWYQQR |
| | | | PGQSVKRLIYYTSSLHSGVPDRFSGSGSGTDYTLKISRVE |
| | | | AEDVGVYYCQQYSKLPRTFGGGTKVEIK |
| SEQ ID NO: 3035 | VL | C-H-VL.36 | EITMTQSPAFMSATPGDKVNISCSASQGISNYLNWYQQ |
| | | | KPGEAVKFIIYYTSSLHSGIPPRFSGSGYGTDYTLTINNIE |
| | | | SEDAAYYYCQQYSKLPRTFGGGTKVEIK |
| SEQ ID NO: 3036 | VL | C-H-VL.37 | DIVMTQTPLSSPVTLGQPASISCSASQGISNYLNWYQQR |
| | | | PGQPVKLLIYYTSSLHSGVPDRFSGSGAGTDYTLKISRV |
| | | | EAEDVGVYYCQQYSKLPRTFGGGTKVEIK |
| SEQ ID NO: 3037 | VL | C-H-VL.38 | EIVMTQSPDFQSVTPKEKVTITCSASQGISNYLNWYQQK |
| | | | PDQSVKLLIYYTSSLHSGVPSRFSGSGSGTDYTLTINSLE |
| | | | AEDAATYYCQQYSKLPRTFGGGTKVEIK |
| SEQ ID NO: 3038 | VL | C-H-VL.39 | EIVMTQTPLSLSITPGEQASISCSASQGISNYLNWYLQKA |
| | | | RPVVKLLIYYTSSLHSGVPDRFSGSGSGTDYTLKISRVEA |
| | | | EDFGVYYCQQYSKLPRTFGGGTKVEIK |
| SEQ ID NO: 3039 | VL | C-H-VL.40 | EIVMTQTPLSLSITPGEQASMSCSASQGISNYLNWYLQK |
| | | | ARPVVKLLIYYTSSLHSGVPDRFSGSGSGTDYTLKISRVE |
| | | | AEDFGVYYCQQYSKLPRTFGGGTKVEIK |

Humanized antibody C Variable HEAVY chain (VH)

| | | | |
|---|---|---|---|
| SEQ ID NO: 3040 | VH | C-H-VH.1 | QVTLKESGPVLVKPTETLTLTCTVSGFSLTAYGVNWVR |
| | | | QPPGKALEWLGMIWGDGNTDYNSALKSRLTISKDNSKS |
| | | | QVVLTMTNMDPVDTATYYCARDRVTATLYAMDYWG |
| | | | QGTLVTVSS |
| SEQ ID NO: 3041 | VH | C-H-VH.2 | QVTLKESGPALVKPTETLTLTCTVSGFSLTAYGVNWVR |
| | | | QPPGKALEWLGMIWGDGNTDYNSALKSRLIISKDNSKS |
| | | | QVVLTMTNMDPVDTATYYCARDRVTATLYAMDYWG |
| | | | QGTLVTVSS |
| SEQ ID NO: 3042 | VH | C-H-VH.3 | QVTLKESGPALVKPTQTLTLTCTVSGFSLTAYGVNWVR |
| | | | QPPGKALEWLGMIWGDGNTDYNSALKSRLTISKDNSKS |
| | | | QVVLTMTNMDPVDTATYYCARDRVTATLYAMDYWG |
| | | | QGTLVTVSS |
| SEQ ID NO: 3043 | VH | C-H-VH.4 | QVQLQESGPGLVKPSGTLSLTCAVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3044 | VH | C-H-VH.5 | QVTLKESGPTLVKPTQTLTLTCTVSGFSLTAYGVNWVR |
| | | | QPPGKALEWLGMIWGDGNTDYNSALKSRLTITKDNSKS |
| | | | QVVLTMTNMDPVDTATYYCARDRVTATLYAMDYWG |
| | | | QGTLVTVSS |
| SEQ ID NO: 3045 | VH | C-H-VH.6 | QVTLKESGPALVKPTQTLTLTCTVSGFSLTAYGVNWVR |
| | | | QPPGKALEWLGMIWGDGNTDYNSALKSRLTITKDNSKS |
| | | | QVVLTMTNMDPVDTATYYCARDRVTATLYAMDYWG |
| | | | QGTLVTVSS |
| SEQ ID NO: 3046 | VH | C-H-VH.7 | QVQLQESGPGLVKPSQTLSLTCTVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3047 | VH | C-H-VH.8 | QVQLQESGPGLVKPSETLSLTCTVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3048 | VH | C-H-VH.9 | QVQLQESGPGLVKPSQTLSLTCAVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3049 | VH | C-H-VH.10 | QVQLQESGPGLVKPSDTLSLTCTVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3050 | VH | C-H-VH.11 | QVQLQESGPGLVKPSQTLSLTCTVSGFSLTAYGVNWVR |
| | | | QHPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3051 | VH | C-H-VH.12 | QVQLQESGPGLVKPSQTLSLTCTVSGFSLTAYGVNWVR |
| | | | QPAGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3052 | VH | C-H-VH.13 | QVQLQESGPGLVKPSQTLSLTCAVSGFSLTAYGVNWVR |
| | | | QPPGKGLEWLGMIWGDGNTDYNSALKSRLTISKDNSKS |
| | | | QVSLKLSSVTAVDTAVYYCARDRVTATLYAMDYWGQ |
| | | | GTLVTVSS |
| SEQ ID NO: 3053 | VH | C-H-VH.14 | QVQLQESGPGLVKPSETLSLTCTVSGFSLTAYGVNWVR |
| | | | QPPGKGLEWLGMIWGDGNTDYNSALKSRLTISKDNSKS |
| | | | HVSLKLSSVTAADTAVYYCARDRVTATLYAMDYWGQ |
| | | | GTLVTVSS |
| SEQ ID NO: 3054 | VH | C-H-VH.15 | QVQLQESGPGLVKPSETLSLTCAVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3055 | VH | C-H-VH.16 | QVQLQESGPGLVKPSQTLSLTCAVYGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3056 | VH | C-H-VH.17 | RVQLQESGPGLVKPSETLSLTCTVSGFSLTAYGVNWVR |
| | | | QPPGKGLEWLGMIWGDGNTDYNSALKSRLTISKDNSKS |
| | | | QVPLKLSSVTAADTAVYYCARDRVTATLYAMDYWGQ |
| | | | GTLVTVSS |
| SEQ ID NO: 3057 | VH | C-H-VH.18 | QVQLQESGPGLVKPSQTLSLTCTVSGFSLTAYGVNWVR |
| | | | QHPGKGLEWLGMIWGDGNTDYNSALKSLLTISKDNSKS |
| | | | QVSLKLSSVTAADTAVYYCARDRVTATLYAMDYWGQ |
| | | | GTLVTVSS |
| SEQ ID NO: 3058 | VH | C-H-VH.19 | QVQLQESGPGLVKPSDTLSLTCAVSGFSLTAYGVNWVR |
| | | | QPPGKGLEWLGMIWGDGNTDYNSALKSRLTISKDNSKS |
| | | | QVSLKLSSVTALDTAVYYCARDRVTATLYAMDYWGQ |
| | | | GTLVTVSS |
| SEQ ID NO: 3059 | VH | C-H-VH.20 | QVQLQESGPGLVKPSDTLSLTCAVSGFSLTAYGVNWVR |
| | | | QPPGKGLEWLGMIWGDGNTDYNSALKSRLTISKDNSKS |
| | | | QVSLKLSSVTAVDTAVYYCARDRVTATLYAMDYWGQ |
| | | | GTLVTVSS |
| SEQ ID NO: 3060 | VH | C-H-VH.21 | QVQLQESGSGLVKPSQTLSLTCAVSGFSLTAYGVNWVR |
| | | | QPPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3061 | VH | C-H-VH.22 | EVQLVESGGGLVQPGRSLRLSCTVSGFSLTAYGVNWVR |
| | | | QAPGKGLEWLGMIWGDGNTDYNSALKSRLTISKDNSKS |
| | | | IVYLQMNSLKTEDTAVYYCARDRVTATLYAMDYWGQ |
| | | | GTLVTVSS |
| SEQ ID NO: 3062 | VH | C-H-VH.23 | EVQLVESGGGLVQPGPSLRLSCTVSGFSLTAYGVNWVR |
| | | | QAPGKGLEWLGMIWGDGNTDYNSALKSRLTISKDNSKS |
| | | | IVYLQMNSLKTEDTAVYYCARDRVTATLYAMDYWGQ |
| | | | GTLVTVSS |
| SEQ ID NO: 3063 | VH | C-H-VH.24 | QVQLQESGSGLVKPSQTLSLTCAVSGFSLTAYGVNWVR |
| | | | QSPGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3064 | VH | C-H-VH.25 | QVQLQESGPGLVKPSETLSLTCTVSGFSLTAYGVNWVR |
| | | | QPAGKGLEWLGMIWGDGNTDYNSALKSRLTISKDNSKS |
| | | | QVSLKLSSVTAADTAVYYCARDRVTATLYAMDYWGQ |
| | | | GTLVTVSS |
| SEQ ID NO: 3065 | VH | C-H-VH.26 | EVQLVESGGGLVKPGRSLRLSCTVSGFSLTAYGVNWVR |
| | | | QAPGKGLEWLGMIWGDGNTDYNSALKSRLTISKDNSKS |
| | | | IVYLQMNSLKTEDTAVYYCARDRVTATLYAMDYWGQ |
| | | | GTLVTVSS |
| SEQ ID NO: 3066 | VH | C-H-VH.27 | QVQLQESGPGLVKPSETLSLTCAVYGFSLTAYGVNWVR |
| | | | QPPGKGLEWLGMIWGDGNTDYNSALKSRLTISKDNSKS |
| | | | QVYLKLSSVTAADTAVYYCARDRVTATLYAMDYWGQ |
| | | | GTLVTVSS |
| SEQ ID NO: 3067 | VH | C-H-VH.28 | QVQLQESGPGLVKPSDTLSLTCAVSGFSLTAYGVNWVR |
| | | | QPPGKGLEWLGMIWGDGNTDYNSALKSRLTISKDNSKS |
| | | | QVSLKLSSVTAVDTGVYYCARDRVTATLYAMDYWGQ |
| | | | GTLVTVSS |
| SEQ ID NO: 3068 | VH | C-H-VH.29 | EVQLVESGGGLVQPGGSLRLSCAVSGFSLTAYGVNWV |
| | | | RQAPGKGLEWLGMIWGDGNTDYNSALKSRLTISKDNS |
| | | | KSSVYLQMNSLKTEDTAVYYCARDRVTATLYAMDYW |
| | | | GQGTLVTVSS |
| SEQ ID NO: 3069 | VH | C-H-VH.30 | EVQLVESGGGLVKPGGSLRLSCAVSGFSLTAYGVNWV |
| | | | RQAPGKGLEWLGMIWGDGNTDYNSALKSRLTISKDNS |
| | | | KSTVYLQMNSLKTEDTAVYYCARDRVTATLYAMDYW |
| | | | GQGTLVTVSS |
| SEQ ID NO: 3070 | VH | C-H-VH.31 | QVQLQQSGPGLVKPSQTLSLTCAVSGFSLTAYGVNWVR |
| | | | QSPSRGLEWLGMIWGDGNTDYNSALKSRLTINKDNSKS |
| | | | QVSLQLNSVTPEDTAVYYCARDRVTATLYAMDYWGQ |
| | | | GTLVTVSS |
| SEQ ID NO: 3071 | VH | C-H-VH.32 | QVQLVESGGGLVQPGGSLRLSCSVSGFSLTAYGVNWVR |
| | | | QAPGKGLEWLGMIWGDGNTDYNSALKSRLTISKDNSKS |
| | | | TVYLQMNSLRAEDTAVYYCARDRVTATLYAMDYWGQ |
| | | | GTLVTVSS |
| SEQ ID NO: 3072 | VH | C-H-VH.33 | QVQLQQWGAGLLKPSETLSLTCAVYGFSLTAYGVNWV |
| | | | RQPPGKGLEWLGMIWGDGNTDYNSALKSRLTISKDNSK |
| | | | SQVSLKLSSVTAADTAVYYCARDRVTATLYAMDYWG |
| | | | QGTLVTVSS |
| SEQ ID NO: 3073 | VH | C-H-VH.34 | QVQLVESGGGVVQPGRSLRLSCAVSGFSLTAYGVNWV |
| | | | RQAPGKGLEWLGMIWGDGNTDYNSALKSRLTISKDNST |
| | | | STVFLQMNSLRAEDTAVYYCARDRVTATLYAMDYWG |
| | | | QGTLVTVSS |
| SEQ ID NO: 3074 | VH | C-H-VH.35 | EVQLVESGGGLVQPGGSLRLSCAVSGFSLTAYGVNWV |
| | | | RQAPGKGLEWLGMIWGDGNTDYNSALKSRLTISKDNS |
| | | | KSTVYLQMNSLRAEDTAVYYCARDRVTATLYAMDYW |
| | | | GQGTLVTVSS |
| SEQ ID NO: 3075 | VH | C-H-VH.36 | EVQLVESGGGLVQPGGSLRLSCAVSGFSLTAYGVNWV |
| | | | RQAPGKGLEWLGMIWGDGNTDYNSALKSRLTISKDNA |
| | | | KSSVYLQMNSLRDEDTAVYYCARDRVTATLYAMDYW |
| | | | GQGTLVTVSS |
| SEQ ID NO: 3076 | VH | C-H-VH.37 | EVQLLESGGGLVQPGGSLRLSCAVSGFSLTAYGVNWVR |
| | | | QAPGKGLEWLGMIWGDGNTDYNSALKSRLTISKDNSKS |
| | | | TVYLQMNSLRAEDTAVYYCARDRVTATLYAMDYWGQ |
| | | | GTLVTVSS |
| SEQ ID NO: 3077 | VH | C-H-VH.38 | QVQLVESGGGLVKPGGSLRLSCAVSGFSLTAYGVNWV |
| | | | RQAPGKGLEWLGMIWGDGNTDYNSALKSRLTISKDNA |
| | | | KSSVYLQMNSLRAEDTAVYYCARDRVTATLYAMDYW |
| | | | GQGTLVTVSS |
| SEQ ID NO: 3078 | VH | C-H-VH.39 | EVQLVESGGGLVQPGGSLKLSCAVSGFSLTAYGVNWV |
| | | | RQASGKGLEWLGMIWGDGNTDYNSALKSRLTISKDNS |
| | | | KSTVYLQMNSLKTEDTAVYYCARDRVTATLYAMDYW |
| | | | GQGTLVTVSS |
| SEQ ID NO: 3079 | VH | C-H-VH.40 | QVQLLESGGGLVKPGGSLRLSCAVSGFSLTAYGVNWV |
| | | | RQAPGKGLEWLGMIWGDGNTDYNSALKSRLTISKDNA |
| | | | KSSVYLQMNSLRAEDTAVYYCARDRVTATLYAMDYW |
| | | | GQGTLVTVSS |
| SEQ ID NO: 3080 | VH | C-H-VH.41 | QVQLVESGGGVVQPGRSLRLSCAVSGFSLTAYGVNWV |
| | | | RQAPGKGLEWLGMIWGDGNTDYNSALKSRLTISKDNS |
| | | | KSTVYLQMNSLRAEDTAVYYCARDRVTATLYAMDYW |
| | | | GQGTLVTVSS |
| SEQ ID NO: 3081 | VH | C-H-VH.42 | QVQLVESGGGVVQPGRSLRLSCAVSGFSLTAYGVNWV |
| | | | RQAPGKGLEWLGMIWGDGNTDYNSALKSRLTISKDNS |
| | | | KSRVYLQMNSLRAEDTAVYYCARDRVTATLYAMDYW |
| | | | GQGTLVTVSS |
| SEQ ID NO: 3082 | VH | C-H-VH.43 | QVQLVESGGGVVQPGRSLRLSCAVSGFSLTAYGVNWV |
| | | | RQAPGKGLEWLGMIWGDGNTDYNSALKSRLAISKDNS |
| | | | KSTVYLQMNSLRAEDTAVYYCARDRVTATLYAMDYW |
| | | | GQGTLVTVSS |
| SEQ ID NO: 3083 | VH | C-H-VH.44 | QVQLVESGGGVVQPGGSLRLSCAVSGFSLTAYGVNWV |
| | | | RQAPGKGLEWLGMIWGDGNTDYNSALKSRLTISKDNS |
| | | | KSTVYLQMNSLRAEDTAVYYCARDRVTATLYAMDYW |
| | | | GQGTLVTVSS |
| SEQ ID NO: 3084 | VH | C-H-VH.45 | EVQLVESGGGLVQPGGSLRLSCAVSGFSLTAYGVNWV |
| | | | RQAPGKGLEWLGMIWGDGNTDYNSALKSRLTISKDNA |
| | | | KSTVYLQMNSLRAEDTAVYYCARDRVTATLYAMDYW |
| | | | GQGTLVTVSS |
| SEQ ID NO: 3085 | VH | C-H-VH.46 | EVQLVESGGGLVQPGGSLRLSCAVSGFSLTAYGVNWV |
| | | | RQAPGKGLEWLGMIWGDGNTDYNSALKSRLTISKDNA |
| | | | KSSVYLQMNSLRAEDTAVYYCARDRVTATLYAMDYW |
| | | | GQGTLVTVSS |
| SEQ ID NO: 3086 | VH | C-H-VH.47 | EVQLVESGGVVVQPGGSLRLSCAVSGFSLTAYGVNWV |
| | | | RQAPGKGLEWLGMIWGDGNTDYNSALKSRLTISKDNS |
| | | | KSSVYLQMNSLRTEDTALYYCARDRVTATLYAMDYW |
| | | | GQGTLVTVSS |
| SEQ ID NO: 3087 | VH | C-H-VH.48 | EVQLVESGGGLVQPGGSLRLSCAVSGFSLTAYGVNWV |
| | | | RQAPGKGLEWLGMIWGDGNTDYNSALKSRLTISKHNS |
| | | | KSTVYLQMNSLRAEDTAVYYCARDRVTATLYAMDYW |
| | | | GQGTLVTVSS |
| SEQ ID NO: 3088 | VH | C-H-VH.49 | EVQLVESGGGLVKPGGSLRLSCAVSGFSLTAYGVNWV |
| | | | RQAPGKGLEWLGMIWGDGNTDYNSALKSRLTISKDNA |
| | | | KSSVYLQMNSLRAEDTAVYYCARDRVTATLYAMDYW |
| | | | GQGTLVTVSS |
| SEQ ID NO: 3089 | VH | C-H-VH.50 | EVQLVESGGGLIQPGGSLRLSCAVSGFSLTAYGVNWVR |
| | | | QPPGKGLEWLGMIWGDGNTDYNSALKSRLTISKDNSKS |
| | | | TVYLQMNSLRAEDTAVYYCARDRVTATLYAMDYWGQ |
| | | | GTLVTVSS |

TABLE 42C
Exemplary molecules or agents that comprise a first binder that binds to a first target
molecule, wherein the exemplary first binder comprises anti-TCRβ V5 antibodies. Exemplary first
target molecule comprises TCRVB 5 (e.g., TCRVB 5-1). Exemplary anti-TCRβ V5 antibodies
include, but are not limited to, BLM0272, which binds to human TRBV5-1. CDRs are underlined.
BLM0272, also referred to as BJ1307 or BJ1309
Binds to human TCRVβ 5-1

| | | |
|---|---|---|
| SEQ ID NO: 3338 | Heavy chain | QVQLVQSGAEVKKPGASVKVSCKASGYTFTDYNIHWVRQAPGQG |
| | (VH-CH) | LEWMGYINPYNGRTGYNQKFKARVTMTVDKSTSTAYMELSSLRS |
| | | EDTAVYYCARWDGSSYFDYWGQGTLVTVSSASTKGPSVFPLAPSS |
| | | KSTSGGTAALGCLVKDYFPEPVTVSWNSGALTSGVHTFPAVLQSS |
| | | GLYSLSSVVTVPSSSLGTQTYICNVNHKPSNTKVDKRVEPKSCDKT |
| | | HTCPPCPAPELLGGPSVFLFPPKPKDTLMISRTPEVTCVVVDVSHED |
| | | PEVKFNWYVDGVEVHNAKTKPREEQYASTYRVVSVLTVLHQDW |
| | | LNGKEYKCKVSNKALPAPIEKTISKAKGQPREPQVCTLPPSREEMT |
| | | KNQVSLSCAVKGFYPSDIAVEWESNGQPENNYKTTPPVLDSDGSFF |
| | | LVSKLTVDKSRWQQGNVFSCSVMHEALHNHYTQKSLSLSPGK |
| SEQ ID NO: 4705 | Heavy chain | QVQLVQSGAEVKKPGASVKVSCKASGYTFTDYNIHWVRQAPGQG |
| | (VH-CH) | LEWMGYINPYNGRTGYNQKFKARVTMTVDKSTSTAYMELSSLRS |
| | | EDTAVYYCARWDGSSYFDYWGQGTLVTVSSASTKGPSVFPLAPSS |
| | | KSTSGGTAALGCLVKDYFPEPVTVSWNSGALTSGVHTFPAVLQSS |
| | | GLYSLSSVVTVPSSSLGTQTYICNVNHKPSNTKVDKRVEPKSCDKT |
| | | HTCPPCPAPELLGGPSVFLFPPKPKDTLMISRTPEVTCVVVDVSHED |
| | | PEVKFNWYVDGVEVHNAKTKPREEQYASTYRVVSVLTVLHQDW |
| | | LNGKEYKCKVSNKALPAPIEKTISKAKGQPREPQVCTLPPSREEMT |
| | | KNQVSLSCAVKGFYPSDIAVEWESNGQPENNYKTTPPVLDSDGSFF |
| | | LVSKLTVDKSRWQQGNVFSCSVMHEALHNHYTQKSLSLSP |
| SEQ ID NO: 3339 | Light chain (VL- | EIVLTQSPATLSVSPGERATLSCSASSSVSYMHWYQQKPGQAPRPLI |
| | CL) | YEISKLASGIPARFSGSGSGTEYTLTISSLQSEDFAVYYCQQWNYPL |
| | | LTFGQGTKLEIKRTVAAPSVFIFPPSDEQLKSGTASVVCLLNNFYPR |
| | | EAKVQWKVDNALQSGNSQESVTEQDSKDSTYSLSSTLTLSKADYE |
| | | KHKVYACEVTHQGLSSPVTKSFNRGEC |
| SEQ ID NO: 3340 | IL-2 C125A- | APTSSSTKKTQLQLEHLLLDLQMILNGINNYKNPKLTRMLTFKFYM |
| | Heavy Chain | PKKATELKHLQCLEEELKPLEEVLNLAQSKNFHLRPRDLISNINVIV |
| | Constant | LELKGSETTFMCEYADETATIVEFLNRWITFAQSIISTLTGGGGSGG |
| | Domain (Fc | GGSDKTHTCPPCPAPELLGGPSVFLFPPKPKDTLMISRTPEVTCVVV |
| | Region) | DVSHEDPEVKFNWYVDGVEVHNAKTKPREEQYASTYRVVSVLTV |
| | | LHQDWLNGKEYKCKVSNKALPAPIEKTISKAKGQPREPQVYTLPP |
| | | CREEMTKNQVSLWCLVKGFYPSDIAVEWESNGQPENNYKTTPPVL |
| | | DSDGSFFLYSKLTVDKSRWQQGNVFSCSVMHEALHNHYTQKSLSL |
| | | SPGK |
| SEQ ID NO: 4706 | IL-2 C125A- | APTSSSTKKTQLQLEHLLLDLQMILNGINNYKNPKLTRMLTFKFYM |
| | Heavy Chain | PKKATELKHLQCLEEELKPLEEVLNLAQSKNFHLRPRDLISNINVIV |
| | Constant | LELKGSETTFMCEYADETATIVEFLNRWITFAQSIISTLTGGGGSGG |
| | Domain (Fc | GGSDKTHTCPPCPAPELLGGPSVFLFPPKPKDTLMISRTPEVTCVVV |
| | Region) | DVSHEDPEVKFNWYVDGVEVHNAKTKPREEQYASTYRVVSVLTV |
| | | LHQDWLNGKEYKCKVSNKALPAPIEKTISKAKGQPREPQVYTLPP |
| | | CREEMTKNQVSLWCLVKGFYPSDIAVEWESNGQPENNYKTTPPVL |
| | | DSDGSFFLYSKLTVDKSRWQQGNVFSCSVMHEALHNHYTQKSLSL |
| | | SP |
| SEQ ID NO: 3341 | HC CDR1 | GYTFTDYNIH |
| SEQ ID NO: 3342 | HC CDR2 | YINPYNGRTGYNQKFKA |
| SEQ ID NO: 3343 | HC CDR3 | WDGSSYFDY |
| SEQ ID NO: 3344 | LC CDR1 | SASSSVSYMH |
| SEQ ID NO: 3345 | LC CDR2 | EISKLAS |
| SEQ ID NO: 3346 | LC CDR3 | QQWNYPLLT |
| SEQ ID NO: 3445 | VH | QVQLVQSGAEVKKPGASVKVSCKASGYTFTDYNIHWVRQAPGQG |
| | | LEWMGYINPYNGRTGYNQKFKARVTMTVDKSTSTAYMELSSLRS |
| | | EDTAVYYCARWDGSSYFDYWGQGTLVTVSS |
| SEQ ID NO: 3446 | VL | EIVLTQSPATLSVSPGERATLSCSASSSVSYMHWYQQKPGQAPRPLI |
| | | YEISKLASGIPARFSGSGSGTEYTLTISSLQSEDFAVYYCQQWNYPL |
| | | LTFGQGTKLEIK |
| SEQ ID NO: 3649 | Heavy chain | ASTKGPSVFPLAPSSKSTSGGTAALGCLVKDYFPEPVTVSWNSGAL |
| | constant region | TSGVHTFPAVLQSSGLYSLSSVVTVPSSSLGTQTYICNVNHKPSNTK |
| | (CH1-CH2-CH3) | VDKRVEPKSCDKTHTCPPCPAPELLGGPSVFLFPPKPKDTLMISRTP |
| | Hole | EVTCVVVDVSHEDPEVKFNWYVDGVEVHNAKTKPREEQYASTYR |
| | | VVSVLTVLHQDWLNGKEYKCKVSNKALPAPIEKTISKAKGQPREP |
| | | QVCTLPPSREEMTKNQVSLSCAVKGFYPSDIAVEWESNGQPENNY |
| | | KTTPPVLDSDGSFFLVSKLTVDKSRWQQGNVFSCSVMHEALHNHY |
| | | TQKSLSLSPGK |
| SEQ ID NO: 3451 | Heavy chain | ASTKGPSVFPLAPSSKSTSGGTAALGCLVKDYFPEPVTVSWNSGAL |
| | constant region | TSGVHTFPAVLQSSGLYSLSSVVTVPSSSLGTQTYICNVNHKPSNTK |
| | (CH1-CH2-CH3) | VDKRVEPKSCDKTHTCPPCPAPELLGGPSVFLFPPKPKDTLMISRTP |
| | Hole | EVTCVVVDVSHEDPEVKFNWYVDGVEVHNAKTKPREEQYASTYR |
| | | VVSVLTVLHQDWLNGKEYKCKVSNKALPAPIEKTISKAKGQPREP |
| | | QVCTLPPSREEMTKNQVSLSCAVKGFYPSDIAVEWESNGQPENNY |
| | | KTTPPVLDSDGSFFLVSKLTVDKSRWQQGNVFSCSVMHEALHNHY |
| | | TQKSLSLSP |
| SEQ ID NO: 3447 | Heavy chain | DKTHTCPPCPAPELLGGPSVFLFPPKPKDTLMISRTPEVTCVVVDVS |
| | hinge-Fc region | HEDPEVKFNWYVDGVEVHNAKTKPREEQYASTYRVVSVLTVLHQ |
| | (CH2-CH3) Hole | DWLNGKEYKCKVSNKALPAPIEKTISKAKGQPREPQVCTLPPSREE |
| | | MTKNQVSLSCAVKGFYPSDIAVEWESNGQPENNYKTTPPVLDSDG |
| | | SFFLVSKLTVDKSRWQQGNVFSCSVMHEALHNHYTQKSLSLSPGK |
| SEQ ID NO: 511 | Heavy chain | DKTHTCPPCPAPELLGGPSVFLFPPKPKDTLMISRTPEVTCVVVDVS |
| | hinge-Fc region | HEDPEVKFNWYVDGVEVHNAKTKPREEQYASTYRVVSVLTVLHQ |
| | (CH2-CH3) Hole | DWLNGKEYKCKVSNKALPAPIEKTISKAKGQPREPQVCTLPPSREE |
| | | MTKNQVSLSCAVKGFYPSDIAVEWESNGQPENNYKTTPPVLDSDG |
| | | SFFLVSKLTVDKSRWQQGNVFSCSVMHEALHNHYTQKSLSLSP |
| SEQ ID NO: 3644 | Light chain | RTVAAPSVFIFPPSDEQLKSGTASVVCLLNNFYPREAKVQWKVDN |
| | constant region | ALQSGNSQESVTEQDSKDSTYSLSSTLTLSKADYEKHKVYACEVT |
| | | HQGLSSPVTKSFNRGEC |
| SEQ ID NO: 2270 | IL-2 C125A | APTSSSTKKTQLQLEHLLLDLQMILNGINNYKNPKLTRMLTFKFYM |
| | | PKKATELKHLQCLEEELKPLEEVLNLAQSKNFHLRPRDLISNINVIV |
| | | LELKGSETTFMCEYADETATIVEFLNRWITFAQSIISTLT |
| SEQ ID NO: 3308 | Linker | GGGGSGGGGS |
| SEQ ID NO: 3648 | Heavy chain | DKTHTCPPCPAPELLGGPSVFLFPPKPKDTLMISRTPEVTCVVVDVS |
| | hinge-Fc region | HEDPEVKFNWYVDGVEVHNAKTKPREEQYASTYRVVSVLTVLHQ |
| | (CH2-CH3) | DWLNGKEYKCKVSNKALPAPIEKTISKAKGQPREPQVYTLPPCREE |
| | knob | MTKNQVSLWCLVKGFYPSDIAVEWESNGQPENNYKTTPPVLDSD |
| | | GSFFLYSKLTVDKSRWQQGNVFSCSVMHEALHNHYTQKSLSLSPGK |
| SEQ ID NO: 508 | Heavy chain | DKTHTCPPCPAPELLGGPSVFLFPPKPKDTLMISRTPEVTCVVVDVS |
| | hinge-Fc region | HEDPEVKFNWYVDGVEVHNAKTKPREEQYASTYRVVSVLTVLHQ |
| | (CH2-CH3) | DWLNGKEYKCKVSNKALPAPIEKTISKAKGQPREPQVYTLPPCREE |
| | knob | MTKNQVSLWCLVKGFYPSDIAVEWESNGQPENNYKTTPPVLDSD |
| | | GSFFLYSKLTVDKSRWQQGNVFSCSVMHEALHNHYTQKSLSLSP |

TABLE 43A
Exemplary molecule comprising an antigen binding domain that binds to a T cell
receptor beta variable region (TCRβV), wherein the exemplary antigen binding domain comprises
anti-TCRβ V6 antibodies. Exemplary antigen binding domain comprises TCRVB 6 (e.g., TCRVB
6-5). Exemplary anti-TCRβ V6 antibodies include, but are not limited to, BKM0186, which binds
to human TCRβV 6-5. CDRs are underlined.
BKM0186, also referred to as BK141 or BK104
Binds to human TCRVβ 6-5

| | | |
|---|---|---|
| SEQ ID NO: 4707 | Heavy chain (VH- | QVQLVQSGAEVKKPGSSVKVSCKASGHDFRLTYIHWVRQAP |
| | CH) | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKRVEPKSCDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVCTLPPSREEMTKNQVSLSCAVKGFYP |
| | | SDIAVEWESNGQPENNYKTTPPVLDSDGSFFLVSKLTVDKSR |
| | | WQQGNVFSCSVMHEALHNHYTQKSLSLSPGK |
| SEQ ID NO: 3348 | Heavy chain (VH- | QVQLVQSGAEVKKPGSSVKVSCKASGHDFRLTYIHWVRQAP |
| | CH) | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKRVEPKSCDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVCTLPPSREEMTKNQVSLSCAVKGFYP |
| | | SDIAVEWESNGQPENNYKTTPPVLDSDGSFFLVSKLTVDKSR |
| | | WQQGNVFSCSVMHEALHNHYTQKSLSLSP |
| SEQ ID NO: 3349 | Light chain (VL-CL) | DIQMTQSPSFLSASVGDRVTITCKASQNVADRVVWHQQKPG |
| | | KAPKALIYSSSHRYKGVPSRFSGSGSGTEFTLTISSLQPEDFAT |
| | | YFCQQFKSYPLTFGQGTKLEIKRTVAAPSVFIFPPSDEQLKSGT |
| | | ASVVCLLNNFYPREAKVQWKVDNALQSGNSQESVTEQDSKD |
| | | STYSLSSTLTLSKADYEKHK VYACEVTHQGLSSPVTKSFNRG |
| | | EC |
| SEQ ID NO: 3340 | IL-2 C125A-Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGSGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVYTLPPCREEMTKNQVSLWCL VKGFY |
| | | PSDIAVEWESNGQPENNYKTTPPVLDSDGSFFLYSKLTVDKS |
| | | RWQQGNVFSCSVMHEALHNHYTQKSLSLSPGK |
| SEQ ID NO: 4706 | IL-2 C125A-Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGSGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVYTLPPCREEMTKNQVSLWCLVKGFY |
| | | PSDIAVEWESNGQPENNYKTTPPVLDSDGSFFLYSKLTVDKS |
| | | RWQQGNVFSCSVMHEALHNHYTQKSLSLSP |
| SEQ ID NO: 3351 | HC CDR1 | GHDFRLTYIH |
| SEQ ID NO: 3352 | HC CDR2 | RVSAGSGNVKYNEKFKG |
| SEQ ID NO: 3353 | HC CDR3 | SYYSYDVLDY |
| SEQ ID NO: 3354 | LC CDR1 | KASQNVADRVV |
| SEQ ID NO: 3355 | LC CDR2 | SSSHRYK |
| SEQ ID NO: 3356 | LC CDR3 | QQFKSYPLT |
| SEQ ID NO: 1346 | VH | QVQLVQSGAEVKKPGSSVKVSCKASGHDFRLTYIHWVRQAPG |
| | | QGLEWMGRVSAGSGNVKYNEKFKGRVTITADTSTSTAYMELS |
| | | SLRSEDTAVYYCAVSYYSYDVLDYWGQGTTVTVSS |
| SEQ ID NO: 1349 | VL | DIQMTQSPSFLSASVGDRVTITCKASQNVADRVVWHQQKPGK |
| | | APKALIYSSSHRYKGVPSRFSGSGSGTEFTLTISSLQPEDFATYF |
| | | CQQFKSYPLTFGQGTKLEIK |
| SEQ ID NO: 3649 | Heavy chain constant | ASTKGPSVFPLAPSSKSTSGGTAALGCLVKDYFPEPVTVSWNS |
| | region (CH1-CH2- | GALTSGVHTFPAVLQSSGLYSLSSVVTVPSSSLGTQTYICNVNH |
| | CH3) Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSPGK |
| SEQ ID NO: 3452 | Heavy chain constant | ASTKGPSVFPLAPSSKSTSGGTAALGCLVKDYFPEPVTVSWNS |
| | region (CH1-CH2- | GALTSGVHTFPAVLQSSGLYSLSSVVTVPSSSLGTQTYICNVNH |
| | CH3) Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSP |
| SEQ ID NO: 3447 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSPGK |
| SEQ ID NO: 511 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSP |
| SEQ ID NO: 3644 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 2270 | IL-2 C125A | APTSSSTKKTQLQLEHLLLDLQMILNGINNYKNPKLTRMLTFK |
| | | FYMPKKATELKHLQCLEEELKPLEEVLNLAQSKNFHLRPRDLI |
| | | SNINVIVLELKGSETTFMCEYADETATIVEFLNRWITFAQSIIST |
| | | LT |
| SEQ ID NO: 3308 | Linker | GGGGSGGGGS |
| SEQ ID NO: 3648 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCL VKGFYPSDIAVEWESNGQPEN |
| | | NYKTTPPVLDSDGSFFLYSKLTVDKSRWQQGNVFSCSVMHEA |
| | | LHNHYTQKSLSLSPGK |
| SEQ ID NO: 3454 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSP |

TABLE 43B
Exemplary molecule comprising an antigen binding domain that
binds to a T cell receptor beta variable region (TCRβV), wherein
the exemplary antigen binding domain comprises anti-TCRVβ 6
antibodies. Exemplary TCRβV comprises TCRVβ 6 (e.g., TCRVβ 6-5).
Exemplary anti-TCRVβ 6 antibodies include, but are not limited to,
BMM0306, BMM0354, BMM0356, BMM0307, BMM0359, BMM0355, BMM0308,
BMM0360, and BMM0357 which bind to human TCRVβ 6 (e.g., TCRVβ 6-5).
CDRs are underlined.
BMM0306

| | | |
|---|---|---|
| SEQ ID NO: 4707 | Heavy chain (VH- | QVQLVQSGAEVKKPGSSVKVSCKASGHDFRLTYIHWVRQAP |
| | CH) - Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVCTLPPSREEMTKNQVSLSC |
| | | AVKGFYPSDIAVEWESNGQPENNYKTTPPVLDSDGSFFLVS |
| | | KLTVDKSRWQQGNVFSCSVMHEALHNHYTQKSLSLSPGK |
| SEQ ID NO: 3348 | Heavy chain (VH- | QVQLVQSGAEVKKPGSSVKVSCKASGHDFRLTYIHWVRQAP |
| | CH) - Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVCTLPPSREEMTKNQVSLSC |
| | | AVKGFYPSDIAVEWESNGQPENNYKTTPPVLDSDGSFFLVS |
| | | KLTVDKSRWQQGNVFSCSVMHEALHNHYTQKSLSLSP |
| SEQ ID NO: 3349 | Light chain (VL-CL) | DIQMTQSPSFLSASVGDRVTITCKASQNVADRVVWHQQKPG |
| | | KAPKALIYSSSHRYKGVPSRFSGSGSGTEFTLTISSLQPED |
| | | FATYFCQQFKSYPLTFGQGTKLEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| SEQ ID NO: 3420 | IL-15Rαsushi-IL-15 | ITCPPPMSVEHADIWVKSYSLYSRERYICNSGFKRKAGTSS |
| | Heavy Chain Constant | LTECVLNKATNVAHWTTPSLKCIRDPALVHQRPAPPSGGSG |
| | Domain (Fc Region) - | GGGSGGGSGGGGSGGGNWVNVISDLKKIEDLIQSMHIDATL |
| | Knob | YTESDVHPSCKVTAMKCFLLELQVISLESGDASIHDTVENL |
| | | IILANNSLSSNGNVTESGCKECEELEEKNIKEFLQSFVHIV |
| | | QMFINTSGGGGSGGGGSDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVYTLPPCREEMTKNQVSLWC |
| | | LVKGFYPSDIAVEWESNGQPENNYKTTPPVLDSDGSFFLYS |
| | | KLTVDKSRWQQGNVFSCSVMHEALHNHYTQKSLSLSPGK |
| SEQ ID NO: 4703 | IL-15Rαsushi-IL-15 | ITCPPPMSVEHADIWVKSYSLYSRERYICNSGFKRKAGTSS |
| | Heavy Chain Constant | LTECVLNKATNVAHWTTPSLKCIRDPALVHQRPAPPSGGSG |
| | Domain (Fc Region) - | GGGSGGGSGGGGSGGGNWVNVISDLKKIEDLIQSMHIDATL |
| | Knob | YTESDVHPSCKVTAMKCFLLELQVISLESGDASIHDTVENL |
| | | IILANNSLSSNGNVTESGCKECEELEEKNIKEFLQSFVHIV |
| | | QMFINTSGGGGSGGGGSDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVYTLPPCREEMTKNQVSLWC |
| | | LVKGFYPSDIAVEWESNGQPENNYKTTPPVLDSDGSFFLYS |
| | | KLTVDKSRWQQGNVFSCSVMHEALHNHYTQKSLSLSP |
| SEQ ID NO: 3351 | HC CDR1 | GHDFRLTYIH |
| SEQ ID NO: 3421 | HC CDR1 | LTYIH |
| SEQ ID NO: 3702 | HC CDR1 | FRLTYIH |
| SEQ ID NO: 3352 | HC CDR2 | RVSAGSGNVKYNEKFKG |
| SEQ ID NO: 3353 | HC CDR3 | SYYSYDVLDY |
| SEQ ID NO: 3354 | LC CDR1 | KASQNVADRVV |
| SEQ ID NO: 3355 | LC CDR2 | SSSHRYK |
| SEQ ID NO: 3356 | LC CDR3 | QQFKSYPLT |
| SEQ ID NO: 1346 | VH | QVQLVQSGAEVKKPGSSVKVSCKASGHDFRLTYIHWVRQAP |
| | | GQGLEWMGRVSAGSGNVKYNEKFKGRVTITADTSTSTAYME |
| | | LSSLRSEDTAVYYCAVSYYSYDVLDYWGQGTTVTVSS |
| SEQ ID NO: 1349 | VL | DIQMTQSPSFLSASVGDRVTITCKASQNVADRVVWHQQKPG |
| | | KAPKALIYSSSHRYKGVPSRFSGSGSGTEFTLTISSLQPED |
| | | FATYFCQQFKSYPLTFGQGTKLEIK |
| SEQ ID NO: 3649 | Heavy chain constant | ASTKGPSVFPLAPSSKSTSGGTAALGCLVKDYFPEPVTVSW |
| | region (CH1-CH2- | NSGALTSGVHTFPAVLQSSGLYSLSSVVTVPSSSLGTQTYI |
| | CH3) Hole | CNVNHKPSNTKVDKRVEPKSCDKTHTCPPCPAPELLGGPSV |
| | | FLFPPKPKDTLMISRTPEVTCVVVDVSHEDPEVKFNWYVDG |
| | | VEVHNAKTKPREEQYASTYRVVSVLTVLHQDWLNGKEYKCK |
| | | VSNKALPAPIEKTISKAKGQPREPQVCTLPPSREEMTKNQV |
| | | SLSCAVKGFYPSDIAVEWESNGQPENNYKTTPPVLDSDGSF |
| | | FLVSKLTVDKSRWQQGNVFSCSVMHEALHNHYTQKSLSLSP |
| | | GK |
| SEQ ID NO: 3452 | Heavy chain constant | ASTKGPSVFPLAPSSKSTSGGTAALGCLVKDYFPEPVTVSW |
| | region (CH1-CH2- | NSGALTSGVHTFPAVLQSSGLYSLSSVVTVPSSSLGTQTYI |
| | CH3) Hole | CNVNHKPSNTKVDKRVEPKSCDKTHTCPPCPAPELLGGPSV |
| | | FLFPPKPKDTLMISRTPEVTCVVVDVSHEDPEVKFNWYVDG |
| | | VEVHNAKTKPREEQYASTYRVVSVLTVLHQDWLNGKEYKCK |
| | | VSNKALPAPIEKTISKAKGQPREPQVCTLPPSREEMTKNQV |
| | | SLSCAVKGFYPSDIAVEWESNGQPENNYKTTPPVLDSDGSF |
| | | FLVSKLTVDKSRWQQGNVFSCSVMHEALHNHYTQKSLSLSP |
| SEQ ID NO: 3447 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | Hole | VSVLTVLHQDWLNGKEYKCKVSNKALPAPIEKTISKAKGQP |
| | | REPQVCTLPPSREEMTKNQVSLSCAVKGFYPSDIAVEWESN |
| | | GQPENNYKTTPPVLDSDGSFFLVSKLTVDKSRWQQGNVFSC |
| | | SVMHEALHNHYTQKSLSLSPGK |
| SEQ ID NO: 511 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | Hole | VSVLTVLHQDWLNGKEYKCKVSNKALPAPIEKTISKAKGQP |
| | | REPQVCTLPPSREEMTKNQVSLSCAVKGFYPSDIAVEWESN |
| | | GQPENNYKTTPPVLDSDGSFFLVSKLTVDKSRWQQGNVFSC |
| | | SVMHEALHNHYTQKSLSLSP |
| SEQ ID NO: 3644 | Light chain constant | RTVAAPSVFIFPPSDEQLKSGTASVVCLLNNFYPREAKVQW |
| | region | KVDNALQSGNSQESVTEQDSKDSTYSLSSTLTLSKADYEKH |
| | | KVYACEVTHQGLSSPVTKSFNRGEC |
| SEQ ID NO: 3472 | IL-15Rαsushi | ITCPPPMSVEHADIWVKSYSLYSRERYICNSGFKRKAGTSS |
| | (AA 31-108 of | LTECVLNKATNVAHWTTPSLKCIRDPALVHQRPAPPS |
| | IL-15Ralpha) | |
| SEQ ID NO: 3674 | Linker | GGSGGGGSGGGSGGGGSGGG |
| SEQ ID NO: 2170 | AA 49-162 of IL-15 | NWVNVISDLKKIEDLIQSMHIDATLYTESDVHPSCKVTAMK |
| | | CFLLELQVISLESGDASIHDTVENLIILANNSLSSNGNVTE |
| | | SGCKECEELEEKNIKEFLQSFVHIVQMFINTS |
| SEQ ID NO: 3474 | IL-15Rαsushi-IL-15 | ITCPPPMSVEHADIWVKSYSLYSRERYICNSGFKRKAGTSS |
| | (AA 31-108 of IL- | LTECVLNKATNVAHWTTPSLKCIRDPALVHQRPAPPSGGSG |
| | 15Ralpha-Linker-AA | GGGSGGGSGGGGSGGGNWVNVISDLKKIEDLIQSMHIDATL |
| | 31-108 of IL- | YTESDVHPSCKVTAMKCFLLELQVISLESGDASIHDTVENL |
| | 15Ralpha) | IILANNSLSSNGNVTESGCKECEELEEKNIKEFLQSFVHIV |
| | | QMFINTS |
| SEQ ID NO: 3308 | Linker | GGGGSGGGGS |
| SEQ ID NO: 3648 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | knob | VSVLTVLHQDWLNGKEYKCKVSNKALPAPIEKTISKAKGQP |
| | | REPQVYTLPPCREEMTKNQVSLWCLVKGFYPSDIAVEWESN |
| | | GQPENNYKTTPPVLDSDGSFFLYSKLTVDKSRWQQGNVFSC |
| | | SVMHEALHNHYTQKSLSLSPGK |
| SEQ ID NO: 508 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | knob | VSVLTVLHQDWLNGKEYKCKVSNKALPAPIEKTISKAKGQP |
| | | REPQVYTLPPCREEMTKNQVSLWCLVKGFYPSDIAVEWESN |
| | | GQPENNYKTTPPVLDSDGSFFLYSKLTVDKSRWQQGNVFSC |
| | | SVMHEALHNHYTQKSLSLSP |

BMM0354

| | | |
|---|---|---|
| SEQ ID NO: 4707 | Heavy chain (VH- | QVQLVQSGAEVKKPGSSVKVSCKASGHDFRLTYIHWVRQAP |
| | CH) - Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVCTLPPSREEMTKNQVSLSC |
| | | AVKGFYPSDIAVEWESNGQPENNYKTTPPVLDSDGSFFLVS |
| | | KLTVDKSRWQQGNVFSCSVMHEALHNHYTQKSLSLSPGK |
| SEQ ID NO: 3348 | Heavy chain (VH- | QVQLVQSGAEVKKPGSSVKVSCKASGHDFRLTYIHWVRQAP |
| | CH) - Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVCTLPPSREEMTKNQVSLSC |
| | | AVKGFYPSDIAVEWESNGQPENNYKTTPPVLDSDGSFFLVS |
| | | KLTVDKSRWQQGNVFSCSVMHEALHNHYTQKSLSLSP |
| SEQ ID NO: 3428 | Light chain (VL-CL) | DIQMTQSPSFLSASVGDRVTITCKASQNVADRVVWHQQKPG |
| | | KAPKALIYAASYLQSGVPSRFSGSGSGTEFTLTISSLQPED |
| | | FATYFCQQFKSYPLTFGQGTKLEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| SEQ ID NO: 3420 | IL-15Rαsushi-IL-15 | ITCPPPMSVEHADIWVKSYSLYSRERYICNSGFKRKAGTSS |
| | Heavy Chain Constant | LTECVLNKATNVAHWTTPSLKCIRDPALVHQRPAPPSGGSG |
| | Domain (Fc Region) - | GGGSGGGSGGGGSGGGNWVNVISDLKKIEDLIQSMHIDATL |
| | Knob | YTESDVHPSCKVTAMKCFLLELQVISLESGDASIHDTVENL |
| | | IILANNSLSSNGNVTESGCKECEELEEKNIKEFLQSFVHIV |
| | | QMFINTSGGGGSGGGGSDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVYTLPPCREEMTKNQVSLWC |
| | | LVKGFYPSDIAVEWESNGQPENNYKTTPPVLDSDGSFFLYS |
| | | KLTVDKSRWQQGNVFSCSVMHEALHNHYTQKSLSLSPGK |
| SEQ ID NO: 4703 | IL-15Rαsushi-IL-15 | ITCPPPMSVEHADIWVKSYSLYSRERYICNSGFKRKAGTSS |
| | Heavy Chain Constant | LTECVLNKATNVAHWTTPSLKCIRDPALVHQRPAPPSGGSG |
| | Domain (Fc Region) - | GGGSGGGSGGGGSGGGNWVNVISDLKKIEDLIQSMHIDATL |
| | Knob | YTESDVHPSCKVTAMKCFLLELQVISLESGDASIHDTVENL |
| | | IILANNSLSSNGNVTESGCKECEELEEKNIKEFLQSFVHIV |
| | | QMFINTSGGGGSGGGGSDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVYTLPPCREEMTKNQVSLWC |
| | | LVKGFYPSDIAVEWESNGQPENNYKTTPPVLDSDGSFFLYS |
| | | KLTVDKSRWQQGNVFSCSVMHEALHNHYTQKSLSLSP |
| SEQ ID NO: 3351 | HC CDR1 | GHDFRLTYIH |
| SEQ ID NO: 3421 | HC CDR1 | LTYIH |
| SEQ ID NO: 3702 | HC CDR1 | FRLTYIH |
| SEQ ID NO: 3352 | HC CDR2 | RVSAGSGNVKYNEKFKG |
| SEQ ID NO: 3353 | HC CDR3 | SYYSYDVLDY |
| SEQ ID NO: 3354 | LC CDR1 | KASQNVADRVV |
| SEQ ID NO: 3443 | LC CDR2 | AASYLQS |
| SEQ ID NO: 3356 | LC CDR3 | QQFKSYPLT |
| SEQ ID NO: 1346 | VH | QVQLVQSGAEVKKPGSSVKVSCKASGHDFRLTYIHWVRQAP |
| | | GQGLEWMGRVSAGSGNVKYNEKFKGRVTITADTSTSTAYME |
| | | LSSLRSEDTAVYYCAVSYYSYDVLDYWGQGTTVTVSS |
| SEQ ID NO: 3475 | VL | DIQMTQSPSFLSASVGDRVTITCKASQNVADRVVWHQQKPG |
| | | KAPKALIYAASYLQSGVPSRFSGSGSGTEFTLTISSLQPED |
| | | FATYFCQQFKSYPLTFGQGTKLEIK |
| SEQ ID NO: 3649 | Heavy chain constant | ASTKGPSVFPLAPSSKSTSGGTAALGCLVKDYFPEPVTVSW |
| | region (CH1-CH2- | NSGALTSGVHTFPAVLQSSGLYSLSSVVTVPSSSLGTQTYI |
| | CH3) Hole | CNVNHKPSNTKVDKRVEPKSCDKTHTCPPCPAPELLGGPSV |
| | | FLFPPKPKDTLMISRTPEVTCVVVDVSHEDPEVKFNWYVDG |
| | | VEVHNAKTKPREEQYASTYRVVSVLTVLHQDWLNGKEYKCK |
| | | VSNKALPAPIEKTISKAKGQPREPQVCTLPPSREEMTKNQV |
| | | SLSCAVKGFYPSDIAVEWESNGQPENNYKTTPPVLDSDGSF |
| | | FLVSKLTVDKSRWQQGNVFSCSVMHEALHNHYTQKSLSLSP |
| | | GK |
| SEQ ID NO: 3452 | Heavy chain constant | ASTKGPSVFPLAPSSKSTSGGTAALGCLVKDYFPEPVTVSW |
| | region (CH1-CH2- | NSGALTSGVHTFPAVLQSSGLYSLSSVVTVPSSSLGTQTYI |
| | CH3) Hole | CNVNHKPSNTKVDKRVEPKSCDKTHTCPPCPAPELLGGPSV |
| | | FLFPPKPKDTLMISRTPEVTCVVVDVSHEDPEVKFNWYVDG |
| | | VEVHNAKTKPREEQYASTYRVVSVLTVLHQDWLNGKEYKCK |
| | | VSNKALPAPIEKTISKAKGQPREPQVCTLPPSREEMTKNQV |
| | | SLSCAVKGFYPSDIAVEWESNGQPENNYKTTPPVLDSDGSF |
| | | FLVSKLTVDKSRWQQGNVFSCSVMHEALHNHYTQKSLSLSP |
| SEQ ID NO: 3447 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | Hole | VSVLTVLHQDWLNGKEYKCKVSNKALPAPIEKTISKAKGQP |
| | | REPQVCTLPPSREEMTKNQVSLSCAVKGFYPSDIAVEWESN |
| | | GQPENNYKTTPPVLDSDGSFFLVSKLTVDKSRWQQGNVFSC |
| | | SVMHEALHNHYTQKSLSLSPGK |
| SEQ ID NO: 511 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | Hole | VSVLTVLHQDWLNGKEYKCKVSNKALPAPIEKTISKAKGQP |
| | | REPQVCTLPPSREEMTKNQVSLSCAVKGFYPSDIAVEWESN |
| | | GQPENNYKTTPPVLDSDGSFFLVSKLTVDKSRWQQGNVFSC |
| | | SVMHEALHNHYTQKSLSLSP |
| SEQ ID NO: 3644 | Light chain constant | RTVAAPSVFIFPPSDEQLKSGTASVVCLLNNFYPREAKVQW |
| | region | KVDNALQSGNSQESVTEQDSKDSTYSLSSTLTLSKADYEKH |
| | | KVYACEVTHQGLSSPVTKSFNRGEC |
| SEQ ID NO: 3472 | IL-15Rαsushi | ITCPPPMSVEHADIWVKSYSLYSRERYICNSGFKRKAGTSS |
| | (AA 31-108 of | LTECVLNKATNVAHWTTPSLKCIRDPALVHQRPAPPS |
| | IL-15Ralpha) | |
| SEQ ID NO: 3473 | Linker | GGSGGGGSGGGSGGGGSGGG |
| SEQ ID NO: 2170 | AA 49-162 of IL-15 | NWVNVISDLKKIEDLIQSMHIDATLYTESDVHPSCKVTAMK |
| | | CFLLELQVISLESGDASIHDTVENLIILANNSLSSNGNVTE |
| | | SGCKECEELEEKNIKEFLQSFVHIVQMFINTS |
| SEQ ID NO: 3474 | IL-15Rαsushi-IL-15 | ITCPPPMSVEHADIWVKSYSLYSRERYICNSGFKRKAGTSS |
| | (AA 31-108 of IL- | LTECVLNKATNVAHWTTPSLKCIRDPALVHQRPAPPSGGSG |
| | 15Ralpha-Linker-AA | GGGSGGGSGGGGSGGGNWVNVISDLKKIEDLIQSMHIDATL |
| | 31-108 of IL- | YTESDVHPSCKVTAMKCFLLELQVISLESGDASIHDTVENL |
| | 15Ralpha) | IILANNSLSSNGNVTESGCKECEELEEKNIKEFLQSFVHIV |
| | | QMFINTS |
| SEQ ID NO: 3308 | Linker | GGGGSGGGGS |
| SEQ ID NO: 3648 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | knob | VSVLTVLHQDWLNGKEYKCKVSNKALPAPIEKTISKAKGQP |
| | | REPQVYTLPPCREEMTKNQVSLWCLVKGFYPSDIAVEWESN |
| | | GQPENNYKTTPPVLDSDGSFFLYSKLTVDKSRWQQGNVFSC |
| | | SVMHEALHNHYTQKSLSLSPGK |
| SEQ ID NO: 508 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | knob | VSVLTVLHQDWLNGKEYKCKVSNKALPAPIEKTISKAKGQP |
| | | REPQVYTLPPCREEMTKNQVSLWCLVKGFYPSDIAVEWESN |
| | | GQPENNYKTTPPVLDSDGSFFLYSKLTVDKSRWQQGNVFSC |
| | | SVMHEALHNHYTQKSLSLSP |

BMM0356

| | | |
|---|---|---|
| SEQ ID NO: 4707 | Heavy chain (VH- | QVQLVQSGAEVKKPGSSVKVSCKASGHDFRLTYIHWVRQAP |
| | CH) - Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVCTLPPSREEMTKNQVSLSC |
| | | AVKGFYPSDIAVEWESNGQPENNYKTTPPVLDSDGSFFLVS |
| | | KLTVDKSRWQQGNVFSCSVMHEALHNHYTQKSLSLSPGK |
| SEQ ID NO: 3348 | Heavy chain (VH- | QVQLVQSGAEVKKPGSSVKVSCKASGHDFRLTYIHWVRQAP |
| | CH) - Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVCTLPPSREEMTKNQVSLSC |
| | | AVKGFYPSDIAVEWESNGQPENNYKTTPPVLDSDGSFFLVS |
| | | KLTVDKSRWQQGNVFSCSVMHEALHNHYTQKSLSLSP |
| SEQ ID NO: 3437 | Light chain (VL-CL) | DIQMTQSPSFLSASVGDRVTITCKASQNVADRVVWHQQKPG |
| | | KAPKALIFAASYLQSGVPSRFSGSGSGTEFTLTISSLQPED |
| | | FATYFCQQFKSYPLTFGQGTKLEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| SEQ ID NO: 3420 | IL-15Rαsushi-IL-15 | ITCPPPMSVEHADIWVKSYSLYSRERYICNSGFKRKAGTSS |
| | Heavy Chain Constant | LTECVLNKATNVAHWTTPSLKCIRDPALVHQRPAPPSGGSG |
| | Domain (Fc Region) - | GGGSGGGSGGGGSGGGNWVNVISDLKKIEDLIQSMHIDATL |
| | Knob | YTESDVHPSCKVTAMKCFLLELQVISLESGDASIHDTVENL |
| | | IILANNSLSSNGNVTESGCKECEELEEKNIKEFLQSFVHIV |
| | | QMFINTSGGGGSGGGGSDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVYTLPPCREEMTKNQVSLWC |
| | | LVKGFYPSDIAVEWESNGQPENNYKTTPPVLDSDGSFFLYS |
| | | KLTVDKSRWQQGNVFSCSVMHEALHNHYTQKSLSLSPGK |
| SEQ ID NO: 4703 | IL-15Rαsushi-IL-15 | ITCPPPMSVEHADIWVKSYSLYSRERYICNSGFKRKAGTSS |
| | Heavy Chain Constant | LTECVLNKATNVAHWTTPSLKCIRDPALVHQRPAPPSGGSG |
| | Domain (Fc Region) - | GGGSGGGSGGGGSGGGNWVNVISDLKKIEDLIQSMHIDATL |
| | Knob | YTESDVHPSCKVTAMKCFLLELQVISLESGDASIHDTVENL |
| | | IILANNSLSSNGNVTESGCKECEELEEKNIKEFLQSFVHIV |
| | | QMFINTSGGGGSGGGGSDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVYTLPPCREEMTKNQVSLWC |
| | | LVKGFYPSDIAVEWESNGQPENNYKTTPPVLDSDGSFFLYS |
| | | KLTVDKSRWQQGNVFSCSVMHEALHNHYTQKSLSLSP |
| SEQ ID NO: 3351 | HC CDR1 | GHDFRLTYIH |
| SEQ ID NO: 3421 | HC CDR1 | LTYIH |
| SEQ ID NO: 3702 | HC CDR1 | FRLTYIH |
| SEQ ID NO: 3352 | HC CDR2 | RVSAGSGNVKYNEKFKG |
| SEQ ID NO: 3353 | HC CDR3 | SYYSYDVLDY |
| SEQ ID NO: 3354 | LC CDR1 | KASQNVADRVV |
| SEQ ID NO: 3443 | LC CDR2 | AASYLQS |
| SEQ ID NO: 3356 | LC CDR3 | QQFKSYPLT |
| SEQ ID NO: 1346 | VH | QVQLVQSGAEVKKPGSSVKVSCKASGHDFRLTYIHWVRQAP |
| | | GQGLEWMGRVSAGSGNVKYNEKFKGRVTITADTSTSTAYME |
| | | LSSLRSEDTAVYYCAVSYYSYDVLDYWGQGTTVTVSS |
| SEQ ID NO: 3476 | VL | DIQMTQSPSFLSASVGDRVTITCKASQNVADRVVWHQQKPG |
| | | KAPKALIFAASYLQSGVPSRFSGSGSGTEFTLTISSLQPED |
| | | FATYFCQQFKSYPLTFGQGTKLEIK |
| SEQ ID NO: 3649 | Heavy chain constant | ASTKGPSVFPLAPSSKSTSGGTAALGCLVKDYFPEPVTVSW |
| | region (CH1-CH2- | NSGALTSGVHTFPAVLQSSGLYSLSSVVTVPSSSLGTQTYI |
| | CH3) Hole | CNVNHKPSNTKVDKRVEPKSCDKTHTCPPCPAPELLGGPSV |
| | | FLFPPKPKDTLMISRTPEVTCVVVDVSHEDPEVKFNWYVDG |
| | | VEVHNAKTKPREEQYASTYRVVSVLTVLHQDWLNGKEYKCK |
| | | VSNKALPAPIEKTISKAKGQPREPQVCTLPPSREEMTKNQV |
| | | SLSCAVKGFYPSDIAVEWESNGQPENNYKTTPPVLDSDGSF |
| | | FLVSKLTVDKSRWQQGNVFSCSVMHEALHNHYTQKSLSLSP |
| | | GK |
| SEQ ID NO: 3452 | Heavy chain constant | ASTKGPSVFPLAPSSKSTSGGTAALGCLVKDYFPEPVTVSW |
| | region (CH1-CH2- | NSGALTSGVHTFPAVLQSSGLYSLSSVVTVPSSSLGTQTYI |
| | CH3) Hole | CNVNHKPSNTKVDKRVEPKSCDKTHTCPPCPAPELLGGPSV |
| | | FLFPPKPKDTLMISRTPEVTCVVVDVSHEDPEVKFNWYVDG |
| | | VEVHNAKTKPREEQYASTYRVVSVLTVLHQDWLNGKEYKCK |
| | | VSNKALPAPIEKTISKAKGQPREPQVCTLPPSREEMTKNQV |
| | | SLSCAVKGFYPSDIAVEWESNGQPENNYKTTPPVLDSDGSF |
| | | FLVSKLTVDKSRWQQGNVFSCSVMHEALHNHYTQKSLSLSP |
| SEQ ID NO: 3447 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | Hole | VSVLTVLHQDWLNGKEYKCKVSNKALPAPIEKTISKAKGQP |
| | | REPQVCTLPPSREEMTKNQVSLSCAVKGFYPSDIAVEWESN |
| | | GQPENNYKTTPPVLDSDGSFFLVSKLTVDKSRWQQGNVFSC |
| | | SVMHEALHNHYTQKSLSLSPGK |
| SEQ ID NO: 511 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | Hole | VSVLTVLHQDWLNGKEYKCKVSNKALPAPIEKTISKAKGQP |
| | | REPQVCTLPPSREEMTKNQVSLSCAVKGFYPSDIAVEWESN |
| | | GQPENNYKTTPPVLDSDGSFFLVSKLTVDKSRWQQGNVFSC |
| | | SVMHEALHNHYTQKSLSLSP |
| SEQ ID NO: 3644 | Light chain constant | RTVAAPSVFIFPPSDEQLKSGTASVVCLLNNFYPREAKVQW |
| | region | KVDNALQSGNSQESVTEQDSKDSTYSLSSTLTLSKADYEKH |
| | | KVYACEVTHQGLSSPVTKSFNRGEC |
| SEQ ID NO: 3472 | IL-15Rαsushi | ITCPPPMSVEHADIWVKSYSLYSRERYICNSGFKRKAGTSS |
| | (AA 31-108 of | LTECVLNKATNVAHWTTPSLKCIRDPALVHQRPAPPS |
| | IL-15Ralpha) | |
| SEQ ID NO: 3473 | Linker | GGSGGGGSGGGSGGGGSGGG |
| SEQ ID NO: 2170 | AA 49-162 of IL-15 | NWVNVISDLKKIEDLIQSMHIDATLYTESDVHPSCKVTAMK |
| | | CFLLELQVISLESGDASIHDTVENLIILANNSLSSNGNVTE |
| | | SGCKECEELEEKNIKEFLQSFVHIVQMFINTS |
| SEQ ID NO: 3474 | IL-15Rαsushi-IL-15 | ITCPPPMSVEHADIWVKSYSLYSRERYICNSGFKRKAGTSS |
| | (AA 31-108 of IL- | LTECVLNKATNVAHWTTPSLKCIRDPALVHQRPAPPSGGSG |
| | 15Ralpha-Linker-AA | GGGSGGGSGGGGSGGGNWVNVISDLKKIEDLIQSMHIDATL |
| | 31-108 of IL- | YTESDVHPSCKVTAMKCFLLELQVISLESGDASIHDTVENL |
| | 15Ralpha) | IILANNSLSSNGNVTESGCKECEELEEKNIKEFLQSFVHIV |
| | | QMFINTS |
| SEQ ID NO: 3308 | Linker | GGGGSGGGGS |
| SEQ ID NO: 3648 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | knob | VSVLTVLHQDWLNGKEYKCKVSNKALPAPIEKTISKAKGQP |
| | | REPQVYTLPPCREEMTKNQVSLWCLVKGFYPSDIAVEWESN |
| | | GQPENNYKTTPPVLDSDGSFFLYSKLTVDKSRWQQGNVFSC |
| | | SVMHEALHNHYTQKSLSLSPGK |
| SEQ ID NO: 508 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | knob | VSVLTVLHQDWLNGKEYKCKVSNKALPAPIEKTISKAKGQP |
| | | REPQVYTLPPCREEMTKNQVSLWCLVKGFYPSDIAVEWESN |
| | | GQPENNYKTTPPVLDSDGSFFLYSKLTVDKSRWQQGNVFSC |
| | | SVMHEALHNHYTQKSLSLSP |

BMM0307

| | | |
|---|---|---|
| SEQ ID NO: 4707 | Heavy chain (VH- | QVQLVQSGAEVKKPGSSVKVSCKASGHDFRLTYIHWVRQAP |
| | CH) - Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVCTLPPSREEMTKNQVSLSC |
| | | AVKGFYPSDIAVEWESNGQPENNYKTTPPVLDSDGSFFLVS |
| | | KLTVDKSRWQQGNVFSCSVMHEALHNHYTQKSLSLSPGK |
| SEQ ID NO: 3348 | Heavy chain (VH- | QVQLVQSGAEVKKPGSSVKVSCKASGHDFRLTYIHWVRQAP |
| | CH) - Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVCTLPPSREEMTKNQVSLSC |
| | | AVKGFYPSDIAVEWESNGQPENNYKTTPPVLDSDGSFFLVS |
| | | KLTVDKSRWQQGNVFSCSVMHEALHNHYTQKSLSLSP |
| SEQ ID NO: 3349 | Light chain (VL-CL) | DIQMTQSPSFLSASVGDRVTITCKASQNVADRVVWHQQKPG |
| | | KAPKALIYSSSHRYKGVPSRFSGSGSGTEFTLTISSLQPED |
| | | FATYFCQQFKSYPLTFGQGTKLEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| SEQ ID NO: 3669 | IL-15Rαsushi-IL-15 | ITCPPPMSVEHADIWVKSYSLYSRERYICNSGFKRKAGTSS |
| | Heavy Chain Constant | LTECVLNKATNVAHWTTPSLKCIRDPALVHQRPAPPSGGSG |
| | Domain (Fc Region) - | GGGSGGGSGGGGSGGGNWVNVISDLKKIEDLIQSMHIDATL |
| | Knob | YTESDVHPSCKVTAMKCFLLELQVISLESGDASIHDTVENL |
| | | IILANNSLSSNGNVTESGCKECEELEEKNIKEFLQSFVHIV |
| | | AMFINTSGGGGSGGGGSDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVYTLPPCREEMTKNQVSLWC |
| | | LVKGFYPSDIAVEWESNGQPENNYKTTPPVLDSDGSFFLYS |
| | | KLTVDKSRWQQGNVFSCSVMHEALHNHYTQKSLSLSPGK |
| SEQ ID NO: 4708 | IL-15Rαsushi-IL-15 | ITCPPPMSVEHADIWVKSYSLYSRERYICNSGFKRKAGTSS |
| | Heavy Chain Constant | LTECVLNKATNVAHWTTPSLKCIRDPALVHQRPAPPSGGSG |
| | Domain (Fc Region) - | GGGSGGGSGGGGSGGGNWVNVISDLKKIEDLIQSMHIDATL |
| | Knob | YTESDVHPSCKVTAMKCFLLELQVISLESGDASIHDTVENL |
| | | IILANNSLSSNGNVTESGCKECEELEEKNIKEFLQSFVHIV |
| | | AMFINTSGGGGSGGGGSDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVYTLPPCREEMTKNQVSLWC |
| | | LVKGFYPSDIAVEWESNGQPENNYKTTPPVLDSDGSFFLYS |
| | | KLTVDKSRWQQGNVFSCSVMHEALHNHYTQKSLSLSP |
| SEQ ID NO: 3351 | HC CDR1 | GHDFRLTYIH |
| SEQ ID NO: 3421 | HC CDR1 | LTYIH |
| SEQ ID NO: 3702 | HC CDR1 | FRLTYIH |
| SEQ ID NO: 3352 | HC CDR2 | RVSAGSGNVKYNEKFKG |
| SEQ ID NO: 3353 | HC CDR3 | SYYSYDVLDY |
| SEQ ID NO: 3354 | LC CDR1 | KASQNVADRVV |
| SEQ ID NO: 3355 | LC CDR2 | SSSHRYK |
| SEQ ID NO: 3356 | LC CDR3 | QQFKSYPLT |
| SEQ ID NO: 1346 | VH | QVQLVQSGAEVKKPGSSVKVSCKASGHDFRLTYIHWVRQAP |
| | | GQGLEWMGRVSAGSGNVKYNEKFKGRVTITADTSTSTAYME |
| | | LSSLRSEDTAVYYCAVSYYSYDVLDYWGQGTTVTVSS |
| SEQ ID NO: 1349 | VL | DIQMTQSPSFLSASVGDRVTITCKASQNVADRVVWHQQKPG |
| | | KAPKALIYSSSHRYKGVPSRFSGSGSGTEFTLTISSLQPED |
| | | FATYFCQQFKSYPLTFGQGTKLEIK |
| SEQ ID NO: 3649 | Heavy chain constant | ASTKGPSVFPLAPSSKSTSGGTAALGCLVKDYFPEPVTVSW |
| | region (CH1-CH2- | NSGALTSGVHTFPAVLQSSGLYSLSSVVTVPSSSLGTQTYI |
| | CH3) Hole | CNVNHKPSNTKVDKRVEPKSCDKTHTCPPCPAPELLGGPSV |
| | | FLFPPKPKDTLMISRTPEVTCVVVDVSHEDPEVKFNWYVDG |
| | | VEVHNAKTKPREEQYASTYRVVSVLTVLHQDWLNGKEYKCK |
| | | VSNKALPAPIEKTISKAKGQPREPQVCTLPPSREEMTKNQV |
| | | SLSCAVKGFYPSDIAVEWESNGQPENNYKTTPPVLDSDGSF |
| | | FLVSKLTVDKSRWQQGNVFSCSVMHEALHNHYTQKSLSLSP |
| | | GK |
| SEQ ID NO: 3452 | Heavy chain constant | ASTKGPSVFPLAPSSKSTSGGTAALGCLVKDYFPEPVTVSW |
| | region (CH1-CH2- | NSGALTSGVHTFPAVLQSSGLYSLSSVVTVPSSSLGTQTYI |
| | CH3) Hole | CNVNHKPSNTKVDKRVEPKSCDKTHTCPPCPAPELLGGPSV |
| | | FLFPPKPKDTLMISRTPEVTCVVVDVSHEDPEVKFNWYVDG |
| | | VEVHNAKTKPREEQYASTYRVVSVLTVLHQDWLNGKEYKCK |
| | | VSNKALPAPIEKTISKAKGQPREPQVCTLPPSREEMTKNQV |
| | | SLSCAVKGFYPSDIAVEWESNGQPENNYKTTPPVLDSDGSF |
| | | FLVSKLTVDKSRWQQGNVFSCSVMHEALHNHYTQKSLSLSP |
| SEQ ID NO: 3447 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | Hole | VSVLTVLHQDWLNGKEYKCKVSNKALPAPIEKTISKAKGQP |
| | | REPQVCTLPPSREEMTKNQVSLSCAVKGFYPSDIAVEWESN |
| | | GQPENNYKTTPPVLDSDGSFFLVSKLTVDKSRWQQGNVFSC |
| | | SVMHEALHNHYTQKSLSLSPGK |
| SEQ ID NO: 511 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | Hole | VSVLTVLHQDWLNGKEYKCKVSNKALPAPIEKTISKAKGQP |
| | | REPQVCTLPPSREEMTKNQVSLSCAVKGFYPSDIAVEWESN |
| | | GQPENNYKTTPPVLDSDGSFFLVSKLTVDKSRWQQGNVFSC |
| | | SVMHEALHNHYTQKSLSLSP |
| SEQ ID NO: 3644 | Light chain constant | RTVAAPSVFIFPPSDEQLKSGTASVVCLLNNFYPREAKVQW |
| | region | KVDNALQSGNSQESVTEQDSKDSTYSLSSTLTLSKADYEKH |
| | | KVYACEVTHQGLSSPVTKSFNRGEC |
| SEQ ID NO: 3472 | IL-15Rαsushi | ITCPPPMSVEHADIWVKSYSLYSRERYICNSGFKRKAGTSS |
| | (AA 31-108 of | LTECVLNKATNVAHWTTPSLKCIRDPALVHQRPAPPS |
| | IL-15Ralpha) | |
| SEQ ID NO: 3473 | Linker | GGSGGGGSGGGSGGGGSGGG |
| SEQ ID NO: 3670 | AA 49-162 of IL-15 | NWVNVISDLKKIEDLIQSMHIDATLYTESDVHPSCKVTAMK |
| | | CFLLELQVISLESGDASIHDTVENLIILANNSLSSNGNVTE |
| | | SGCKECEELEEKNIKEFLQSFVHIVAMFINTS |
| SEQ ID NO: 3671 | IL-15Rαsushi-IL-15 | ITCPPPMSVEHADIWVKSYSLYSRERYICNSGFKRKAGTSS |
| | (AA 31-108 of IL- | LTECVLNKATNVAHWTTPSLKCIRDPALVHQRPAPPSGGSG |
| | 15Ralpha-Linker-AA | GGGSGGGSGGGGSGGGNWVNVISDLKKIEDLIQSMHIDATL |
| | 31-108 of IL- | YTESDVHPSCKVTAMKCFLLELQVISLESGDASIHDTVENL |
| | 15Ralpha) | IILANNSLSSNGNVTESGCKECEELEEKNIKEFLQSFVHIV |
| | | AMFINTS |
| SEQ ID NO: 3308 | Linker | GGGGSGGGGS |
| SEQ ID NO: 3648 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | knob | VSVLTVLHQDWLNGKEYKCKVSNKALPAPIEKTISKAKGQP |
| | | REPQVYTLPPCREEMTKNQVSLWCLVKGFYPSDIAVEWESN |
| | | GQPENNYKTTPPVLDSDGSFFLYSKLTVDKSRWQQGNVFSC |
| | | SVMHEALHNHYTQKSLSLSPGK |
| SEQ ID NO: 508 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | knob | VSVLTVLHQDWLNGKEYKCKVSNKALPAPIEKTISKAKGQP |
| | | REPQVYTLPPCREEMTKNQVSLWCLVKGFYPSDIAVEWESN |
| | | GQPENNYKTTPPVLDSDGSFFLYSKLTVDKSRWQQGNVFSC |
| | | SVMHEALHNHYTQKSLSLSP |

BMM0359

| | | |
|---|---|---|
| SEQ ID NO: 4707 | Heavy chain (VH- | QVQLVQSGAEVKKPGSSVKVSCKASGHDFRLTYIHWVRQAP |
| | CH) - Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVCTLPPSREEMTKNQVSLSC |
| | | AVKGFYPSDIAVEWESNGQPENNYKTTPPVLDSDGSFFLVS |
| | | KLTVDKSRWQQGNVFSCSVMHEALHNHYTQKSLSLSPGK |
| SEQ ID NO: 3348 | Heavy chain (VH- | QVQLVQSGAEVKKPGSSVKVSCKASGHDFRLTYIHWVRQAP |
| | CH) - Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVCTLPPSREEMTKNQVSLSC |
| | | AVKGFYPSDIAVEWESNGQPENNYKTTPPVLDSDGSFFLVS |
| | | KLTVDKSRWQQGNVFSCSVMHEALHNHYTQKSLSLSP |
| SEQ ID NO: 3428 | Light chain (VL-CL) | DIQMTQSPSFLSASVGDRVTITCKASQNVADRVVWHQQKPG |
| | | KAPKALIYAASYLQSGVPSRFSGSGSGTEFTLTISSLQPED |
| | | FATYFCQQFKSYPLTFGQGTKLEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| SEQ ID NO: 3669 | IL-15Rαsushi-IL-15 | ITCPPPMSVEHADIWVKSYSLYSRERYICNSGFKRKAGTSS |
| | Heavy Chain Constant | LTECVLNKATNVAHWTTPSLKCIRDPALVHQRPAPPSGGSG |
| | Domain (Fc Region) - | GGGSGGGSGGGGSGGGNWVNVISDLKKIEDLIQSMHIDATL |
| | Knob | YTESDVHPSCKVTAMKCFLLELQVISLESGDASIHDTVENL |
| | | IILANNSLSSNGNVTESGCKECEELEEKNIKEFLQSFVHIV |
| | | AMFINTSGGGGSGGGGSDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVYTLPPCREEMTKNQVSLWC |
| | | LVKGFYPSDIAVEWESNGQPENNYKTTPPVLDSDGSFFLYS |
| | | KLTVDKSRWQQGNVFSCSVMHEALHNHYTQKSLSLSPGK |
| SEQ ID NO: 4708 | IL-15Rαsushi-IL-15 | ITCPPPMSVEHADIWVKSYSLYSRERYICNSGFKRKAGTSS |
| | Heavy Chain Constant | LTECVLNKATNVAHWTTPSLKCIRDPALVHQRPAPPSGGSG |
| | Domain (Fc Region) - | GGGSGGGSGGGGSGGGNWVNVISDLKKIEDLIQSMHIDATL |
| | Knob | YTESDVHPSCKVTAMKCFLLELQVISLESGDASIHDTVENL |
| | | IILANNSLSSNGNVTESGCKECEELEEKNIKEFLQSFVHIV |
| | | AMFINTSGGGGSGGGGSDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVYTLPPCREEMTKNQVSLWC |
| | | LVKGFYPSDIAVEWESNGQPENNYKTTPPVLDSDGSFFLYS |
| | | KLTVDKSRWQQGNVFSCSVMHEALHNHYTQKSLSLSP |
| SEQ ID NO: 3351 | HC CDR1 | GHDFRLTYIH |
| SEQ ID NO: 3421 | HC CDR1 | LTYIH |
| SEQ ID NO: 3702 | HC CDR1 | FRLTYIH |
| SEQ ID NO: 3352 | HC CDR2 | RVSAGSGNVKYNEKFKG |
| SEQ ID NO: 3353 | HC CDR3 | SYYSYDVLDY |
| SEQ ID NO: 3354 | LC CDR1 | KASQNVADRVV |
| SEQ ID NO: 3443 | LC CDR2 | AASYLQS |
| SEQ ID NO: 3356 | LC CDR3 | QQFKSYPLT |
| SEQ ID NO: 1346 | VH | QVQLVQSGAEVKKPGSSVKVSCKASGHDFRLTYIHWVRQAP |
| | | GQGLEWMGRVSAGSGNVKYNEKFKGRVTITADTSTSTAYME |
| | | LSSLRSEDTAVYYCAVSYYSYDVLDYWGQGTTVTVSS |
| SEQ ID NO: 3475 | VL | DIQMTQSPSFLSASVGDRVTITCKASQNVADRVVWHQQKPG |
| | | KAPKALIYAASYLQSGVPSRFSGSGSGTEFTLTISSLQPED |
| | | FATYFCQQFKSYPLTFGQGTKLEIK |
| SEQ ID NO: 3649 | Heavy chain constant | ASTKGPSVFPLAPSSKSTSGGTAALGCLVKDYFPEPVTVSW |
| | region (CH1-CH2- | NSGALTSGVHTFPAVLQSSGLYSLSSVVTVPSSSLGTQTYI |
| | CH3) Hole | CNVNHKPSNTKVDKRVEPKSCDKTHTCPPCPAPELLGGPSV |
| | | FLFPPKPKDTLMISRTPEVTCVVVDVSHEDPEVKFNWYVDG |
| | | VEVHNAKTKPREEQYASTYRVVSVLTVLHQDWLNGKEYKCK |
| | | VSNKALPAPIEKTISKAKGQPREPQVCTLPPSREEMTKNQV |
| | | SLSCAVKGFYPSDIAVEWESNGQPENNYKTTPPVLDSDGSF |
| | | FLVSKLTVDKSRWQQGNVFSCSVMHEALHNHYTQKSLSLSP |
| | | GK |
| SEQ ID NO: 3452 | Heavy chain constant | ASTKGPSVFPLAPSSKSTSGGTAALGCLVKDYFPEPVTVSW |
| | region (CH1-CH2- | NSGALTSGVHTFPAVLQSSGLYSLSSVVTVPSSSLGTQTYI |
| | CH3) Hole | CNVNHKPSNTKVDKRVEPKSCDKTHTCPPCPAPELLGGPSV |
| | | FLFPPKPKDTLMISRTPEVTCVVVDVSHEDPEVKFNWYVDG |
| | | VEVHNAKTKPREEQYASTYRVVSVLTVLHQDWLNGKEYKCK |
| | | VSNKALPAPIEKTISKAKGQPREPQVCTLPPSREEMTKNQV |
| | | SLSCAVKGFYPSDIAVEWESNGQPENNYKTTPPVLDSDGSF |
| | | FLVSKLTVDKSRWQQGNVFSCSVMHEALHNHYTQKSLSLSP |
| SEQ ID NO: 3447 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | Hole | VSVLTVLHQDWLNGKEYKCKVSNKALPAPIEKTISKAKGQP |
| | | REPQVCTLPPSREEMTKNQVSLSCAVKGFYPSDIAVEWESN |
| | | GQPENNYKTTPPVLDSDGSFFLVSKLTVDKSRWQQGNVFSC |
| | | SVMHEALHNHYTQKSLSLSPGK |
| SEQ ID NO: 511 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | Hole | VSVLTVLHQDWLNGKEYKCKVSNKALPAPIEKTISKAKGQP |
| | | REPQVCTLPPSREEMTKNQVSLSCAVKGFYPSDIAVEWESN |
| | | GQPENNYKTTPPVLDSDGSFFLVSKLTVDKSRWQQGNVFSC |
| | | SVMHEALHNHYTQKSLSLSP |
| SEQ ID NO: 3644 | Light chain constant | RTVAAPSVFIFPPSDEQLKSGTASVVCLLNNFYPREAKVQW |
| | region | KVDNALQSGNSQESVTEQDSKDSTYSLSSTLTLSKADYEKH |
| | | KVYACEVTHQGLSSPVTKSFNRGEC |
| SEQ ID NO: 3472 | IL-15Rαsushi | ITCPPPMSVEHADIWVKSYSLYSRERYICNSGFKRKAGTSS |
| | (AA 31-108 of | LTECVLNKATNVAHWTTPSLKCIRDPALVHQRPAPPS |
| | IL-15Ralpha) | |
| SEQ ID NO: 3473 | Linker | GGSGGGGSGGGSGGGGSGGG |
| SEQ ID NO: 3670 | AA 49-162 of IL-15 | NWVNVISDLKKIEDLIQSMHIDATLYTESDVHPSCKVTAMK |
| | | CFLLELQVISLESGDASIHDTVENLIILANNSLSSNGNVTE |
| | | SGCKECEELEEKNIKEFLQSFVHIVAMFINTS |
| SEQ ID NO: 3671 | IL-15Rαsushi-IL-15 | ITCPPPMSVEHADIWVKSYSLYSRERYICNSGFKRKAGTSS |
| | (AA 31-108 of IL- | LTECVLNKATNVAHWTTPSLKCIRDPALVHQRPAPPSGGSG |
| | 15Ralpha-Linker-AA | GGGSGGGSGGGGSGGGNWVNVISDLKKIEDLIQSMHIDATL |
| | 31-108 of IL- | YTESDVHPSCKVTAMKCFLLELQVISLESGDASIHDTVENL |
| | 15Ralpha) | IILANNSLSSNGNVTESGCKECEELEEKNIKEFLQSFVHIV |
| | | AMFINTS |
| SEQ ID NO: 3308 | Linker | GGGGSGGGGS |
| SEQ ID NO: 3648 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | knob | VSVLTVLHQDWLNGKEYKCKVSNKALPAPIEKTISKAKGQP |
| | | REPQVYTLPPCREEMTKNQVSLWCLVKGFYPSDIAVEWESN |
| | | GQPENNYKTTPPVLDSDGSFFLYSKLTVDKSRWQQGNVFSC |
| | | SVMHEALHNHYTQKSLSLSPGK |
| SEQ ID NO: 508 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | knob | VSVLTVLHQDWLNGKEYKCKVSNKALPAPIEKTISKAKGQP |
| | | REPQVYTLPPCREEMTKNQVSLWCLVKGFYPSDIAVEWESN |
| | | GQPENNYKTTPPVLDSDGSFFLYSKLTVDKSRWQQGNVFSC |
| | | SVMHEALHNHYTQKSLSLSP |

BMM0355

| | | |
|---|---|---|
| SEQ ID NO: 4707 | Heavy chain (VH- | QVQLVQSGAEVKKPGSSVKVSCKASGHDFRLTYIHWVRQAP |
| | CH) - Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVCTLPPSREEMTKNQVSLSC |
| | | AVKGFYPSDIAVEWESNGQPENNYKTTPPVLDSDGSFFLVS |
| | | KLTVDKSRWQQGNVFSCSVMHEALHNHYTQKSLSLSPGK |
| SEQ ID NO: 3348 | Heavy chain (VH- | QVQLVQSGAEVKKPGSSVKVSCKASGHDFRLTYIHWVRQAP |
| | CH) - Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVCTLPPSREEMTKNQVSLSC |
| | | AVKGFYPSDIAVEWESNGQPENNYKTTPPVLDSDGSFFLVS |
| | | KLTVDKSRWQQGNVFSCSVMHEALHNHYTQKSLSLSP |
| SEQ ID NO: 3437 | Light chain (VL-CL) | DIQMTQSPSFLSASVGDRVTITCKASQNVADRVVWHQQKPG |
| | | KAPKALIFAASYLQSGVPSRFSGSGSGTEFTLTISSLQPED |
| | | FATYFCQQFKSYPLTFGQGTKLEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| SEQ ID NO: 3669 | IL-15Rαsushi-IL-15 | ITCPPPMSVEHADIWVKSYSLYSRERYICNSGFKRKAGTSS |
| | Heavy Chain Constant | LTECVLNKATNVAHWTTPSLKCIRDPALVHQRPAPPSGGSG |
| | Domain (Fc Region) - | GGGSGGGSGGGGSGGGNWVNVISDLKKIEDLIQSMHIDATL |
| | Knob | YTESDVHPSCKVTAMKCFLLELQVISLESGDASIHDTVENL |
| | | IILANNSLSSNGNVTESGCKECEELEEKNIKEFLQSFVHIV |
| | | AMFINTSGGGGSGGGGSDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVYTLPPCREEMTKNQVSLWC |
| | | LVKGFYPSDIAVEWESNGQPENNYKTTPPVLDSDGSFFLYS |
| | | KLTVDKSRWQQGNVFSCSVMHEALHNHYTQKSLSLSPGK |
| SEQ ID NO: 4708 | IL-15Rαsushi-IL-15 | ITCPPPMSVEHADIWVKSYSLYSRERYICNSGFKRKAGTSS |
| | Heavy Chain Constant | LTECVLNKATNVAHWTTPSLKCIRDPALVHQRPAPPSGGSG |
| | Domain (Fc Region) - | GGGSGGGSGGGGSGGGNWVNVISDLKKIEDLIQSMHIDATL |
| | Knob | YTESDVHPSCKVTAMKCFLLELQVISLESGDASIHDTVENL |
| | | IILANNSLSSNGNVTESGCKECEELEEKNIKEFLQSFVHIV |
| | | AMFINTSGGGGSGGGGSDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVYTLPPCREEMTKNQVSLWC |
| | | LVKGFYPSDIAVEWESNGQPENNYKTTPPVLDSDGSFFLYS |
| | | KLTVDKSRWQQGNVFSCSVMHEALHNHYTQKSLSLSP |
| SEQ ID NO: 3351 | HC CDR1 | GHDFRLTYIH |
| SEQ ID NO: 3421 | HC CDR1 | LTYIH |
| SEQ ID NO: 3702 | HC CDR1 | FRLTYIH |
| SEQ ID NO: 3352 | HC CDR2 | RVSAGSGNVKYNEKFKG |
| SEQ ID NO: 3353 | HC CDR3 | SYYSYDVLDY |
| SEQ ID NO: 3354 | LC CDR1 | KASQNVADRVV |
| SEQ ID NO: 3443 | LC CDR2 | AASYLQS |
| SEQ ID NO: 3356 | LC CDR3 | QQFKSYPLT |
| SEQ ID NO: 1346 | VH | QVQLVQSGAEVKKPGSSVKVSCKASGHDFRLTYIHWVRQAP |
| | | GQGLEWMGRVSAGSGNVKYNEKFKGRVTITADTSTSTAYME |
| | | LSSLRSEDTAVYYCAVSYYSYDVLDYWGQGTTVTVSS |
| SEQ ID NO: 3476 | VL | DIQMTQSPSFLSASVGDRVTITCKASQNVADRVVWHQQKPG |
| | | KAPKALIFAASYLQSGVPSRFSGSGSGTEFTLTISSLQPED |
| | | FATYFCQQFKSYPLTFGQGTKLEIK |
| SEQ ID NO: 3649 | Heavy chain constant | ASTKGPSVFPLAPSSKSTSGGTAALGCLVKDYFPEPVTVSW |
| | region (CH1-CH2- | NSGALTSGVHTFPAVLQSSGLYSLSSVVTVPSSSLGTQTYI |
| | CH3) Hole | CNVNHKPSNTKVDKRVEPKSCDKTHTCPPCPAPELLGGPSV |
| | | FLFPPKPKDTLMISRTPEVTCVVVDVSHEDPEVKFNWYVDG |
| | | VEVHNAKTKPREEQYASTYRVVSVLTVLHQDWLNGKEYKCK |
| | | VSNKALPAPIEKTISKAKGQPREPQVCTLPPSREEMTKNQV |
| | | SLSCAVKGFYPSDIAVEWESNGQPENNYKTTPPVLDSDGSF |
| | | FLVSKLTVDKSRWQQGNVFSCSVMHEALHNHYTQKSLSLSP |
| | | GK |
| SEQ ID NO: 3452 | Heavy chain constant | ASTKGPSVFPLAPSSKSTSGGTAALGCLVKDYFPEPVTVSW |
| | region (CH1-CH2- | NSGALTSGVHTFPAVLQSSGLYSLSSVVTVPSSSLGTQTYI |
| | CH3) Hole | CNVNHKPSNTKVDKRVEPKSCDKTHTCPPCPAPELLGGPSV |
| | | FLFPPKPKDTLMISRTPEVTCVVVDVSHEDPEVKFNWYVDG |
| | | VEVHNAKTKPREEQYASTYRVVSVLTVLHQDWLNGKEYKCK |
| | | VSNKALPAPIEKTISKAKGQPREPQVCTLPPSREEMTKNQV |
| | | SLSCAVKGFYPSDIAVEWESNGQPENNYKTTPPVLDSDGSF |
| | | FLVSKLTVDKSRWQQGNVFSCSVMHEALHNHYTQKSLSLSP |
| SEQ ID NO: 3447 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | Hole | VSVLTVLHQDWLNGKEYKCKVSNKALPAPIEKTISKAKGQP |
| | | REPQVCTLPPSREEMTKNQVSLSCAVKGFYPSDIAVEWESN |
| | | GQPENNYKTTPPVLDSDGSFFLVSKLTVDKSRWQQGNVFSC |
| | | SVMHEALHNHYTQKSLSLSPGK |
| SEQ ID NO: 511 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | Hole | VSVLTVLHQDWLNGKEYKCKVSNKALPAPIEKTISKAKGQP |
| | | REPQVCTLPPSREEMTKNQVSLSCAVKGFYPSDIAVEWESN |
| | | GQPENNYKTTPPVLDSDGSFFLVSKLTVDKSRWQQGNVFSC |
| | | SVMHEALHNHYTQKSLSLSP |
| SEQ ID NO: 3644 | Light chain constant | RTVAAPSVFIFPPSDEQLKSGTASVVCLLNNFYPREAKVQW |
| | region | KVDNALQSGNSQESVTEQDSKDSTYSLSSTLTLSKADYEKH |
| | | KVYACEVTHQGLSSPVTKSFNRGEC |
| SEQ ID NO: 3472 | IL-15Rαsushi | ITCPPPMSVEHADIWVKSYSLYSRERYICNSGFKRKAGTSS |
| | (AA 31-108 of | LTECVLNKATNVAHWTTPSLKCIRDPALVHQRPAPPS |
| | IL-15Ralpha) | |
| SEQ ID NO: 3473 | Linker | GGSGGGGSGGGSGGGGSGGG |
| SEQ ID NO: 3670 | AA 49-162 of IL-15 | NWVNVISDLKKIEDLIQSMHIDATLYTESDVHPSCKVTAMK |
| | | CFLLELQVISLESGDASIHDTVENLIILANNSLSSNGNVTE |
| | | SGCKECEELEEKNIKEFLQSFVHIVAMFINTS |
| SEQ ID NO: 3671 | IL-15Rαsushi-IL-15 | ITCPPPMSVEHADIWVKSYSLYSRERYICNSGFKRKAGTSS |
| | (AA 31-108 of IL- | LTECVLNKATNVAHWTTPSLKCIRDPALVHQRPAPPSGGSG |
| | 15Ralpha-Linker-AA | GGGSGGGSGGGGSGGGNWVNVISDLKKIEDLIQSMHIDATL |
| | 31-108 of IL- | YTESDVHPSCKVTAMKCFLLELQVISLESGDASIHDTVENL |
| | 15Ralpha) | IILANNSLSSNGNVTESGCKECEELEEKNIKEFLQSFVHIV |
| | | AMFINTS |
| SEQ ID NO: 3308 | Linker | GGGGSGGGGS |
| SEQ ID NO: 3648 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | knob | VSVLTVLHQDWLNGKEYKCKVSNKALPAPIEKTISKAKGQP |
| | | REPQVYTLPPCREEMTKNQVSLWCLVKGFYPSDIAVEWESN |
| | | GQPENNYKTTPPVLDSDGSFFLYSKLTVDKSRWQQGNVFSC |
| | | SVMHEALHNHYTQKSLSLSPGK |
| SEQ ID NO: 508 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | knob | VSVLTVLHQDWLNGKEYKCKVSNKALPAPIEKTISKAKGQP |
| | | REPQVYTLPPCREEMTKNQVSLWCLVKGFYPSDIAVEWESN |
| | | GQPENNYKTTPPVLDSDGSFFLYSKLTVDKSRWQQGNVFSC |
| | | SVMHEALHNHYTQKSLSLSP |

BMM0308

| | | |
|---|---|---|
| SEQ ID NO: 4707 | Heavy chain (VH- | QVQLVQSGAEVKKPGSSVKVSCKASGHDFRLTYIHWVRQAP |
| | CH) - Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVCTLPPSREEMTKNQVSLSC |
| | | AVKGFYPSDIAVEWESNGQPENNYKTTPPVLDSDGSFFLVS |
| | | KLTVDKSRWQQGNVFSCSVMHEALHNHYTQKSLSLSPGK |
| SEQ ID NO: 3348 | Heavy chain (VH- | QVQLVQSGAEVKKPGSSVKVSCKASGHDFRLTYIHWVRQAP |
| | CH) - Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVCTLPPSREEMTKNQVSLSC |
| | | AVKGFYPSDIAVEWESNGQPENNYKTTPPVLDSDGSFFLVS |
| | | KLTVDKSRWQQGNVFSCSVMHEALHNHYTQKSLSLSP |
| SEQ ID NO: 3349 | Light chain (VL-CL) | DIQMTQSPSFLSASVGDRVTITCKASQNVADRVVWHQQKPG |
| | | KAPKALIYSSSHRYKGVPSRFSGSGSGTEFTLTISSLQPED |
| | | FATYFCQQFKSYPLTFGQGTKLEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| SEQ ID NO: 3672 | IL-15Rαsushi-IL-15 | ITCPPPMSVEHADIWVKSYSLYSRERYICNSGFKRKAGTSS |
| | Heavy Chain Constant | LTECVLNKATNVAHWTTPSLKCIRDPALVHQRPAPPSGGSG |
| | Domain (Fc Region) - | GGGSGGGSGGGGSGGGNWVNVISDLKKIEDLIQSMHIDATL |
| | Knob | YTESDVHPSCKVTAMKCFLLELQVISLESGDASIHDTVEQL |
| | | IILANNSLSSNGNVTESGCKECEELEEKNIKEFLQSFVHIV |
| | | QMFINTSGGGGSGGGGSDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVYTLPPCREEMTKNQVSLWC |
| | | LVKGFYPSDIAVEWESNGQPENNYKTTPPVLDSDGSFFLYS |
| | | KLTVDKSRWQQGNVFSCSVMHEALHNHYTQKSLSLSPGK |
| SEQ ID NO: 4709 | IL-15Rαsushi-IL-15 | ITCPPPMSVEHADIWVKSYSLYSRERYICNSGFKRKAGTSS |
| | Heavy Chain Constant | LTECVLNKATNVAHWTTPSLKCIRDPALVHQRPAPPSGGSG |
| | Domain (Fc Region) - | GGGSGGGSGGGGSGGGNWVNVISDLKKIEDLIQSMHIDATL |
| | Knob | YTESDVHPSCKVTAMKCFLLELQVISLESGDASIHDTVEQL |
| | | IILANNSLSSNGNVTESGCKECEELEEKNIKEFLQSFVHIV |
| | | QMFINTSGGGGSGGGGSDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVYTLPPCREEMTKNQVSLWC |
| | | LVKGFYPSDIAVEWESNGQPENNYKTTPPVLDSDGSFFLYS |
| | | KLTVDKSRWQQGNVFSCSVMHEALHNHYTQKSLSLSP |
| SEQ ID NO: 3351 | HC CDR1 | GHDFRLTYIH |
| SEQ ID NO: 3421 | HC CDR1 | LTYIH |
| SEQ ID NO: 3702 | HC CDR1 | FRLTYIH |
| SEQ ID NO: 3352 | HC CDR2 | RVSAGSGNVKYNEKFKG |
| SEQ ID NO: 3353 | HC CDR3 | SYYSYDVLDY |
| SEQ ID NO: 3354 | LC CDR1 | KASQNVADRVV |
| SEQ ID NO: 3355 | LC CDR2 | SSSHRYK |
| SEQ ID NO: 3356 | LC CDR3 | QQFKSYPLT |
| SEQ ID NO: 1346 | VH | QVQLVQSGAEVKKPGSSVKVSCKASGHDFRLTYIHWVRQAP |
| | | GQGLEWMGRVSAGSGNVKYNEKFKGRVTITADTSTSTAYME |
| | | LSSLRSEDTAVYYCAVSYYSYDVLDYWGQGTTVTVSS |
| SEQ ID NO: 1349 | VL | DIQMTQSPSFLSASVGDRVTITCKASQNVADRVVWHQQKPG |
| | | KAPKALIYSSSHRYKGVPSRFSGSGSGTEFTLTISSLQPED |
| | | FATYFCQQFKSYPLTFGQGTKLEIK |
| SEQ ID NO: 3649 | Heavy chain constant | ASTKGPSVFPLAPSSKSTSGGTAALGCLVKDYFPEPVTVSW |
| | region (CH1-CH2- | NSGALTSGVHTFPAVLQSSGLYSLSSVVTVPSSSLGTQTYI |
| | CH3) Hole | CNVNHKPSNTKVDKRVEPKSCDKTHTCPPCPAPELLGGPSV |
| | | FLFPPKPKDTLMISRTPEVTCVVVDVSHEDPEVKFNWYVDG |
| | | VEVHNAKTKPREEQYASTYRVVSVLTVLHQDWLNGKEYKCK |
| | | VSNKALPAPIEKTISKAKGQPREPQVCTLPPSREEMTKNQV |
| | | SLSCAVKGFYPSDIAVEWESNGQPENNYKTTPPVLDSDGSF |
| | | FLVSKLTVDKSRWQQGNVFSCSVMHEALHNHYTQKSLSLSP |
| | | GK |
| SEQ ID NO: 3452 | Heavy chain constant | ASTKGPSVFPLAPSSKSTSGGTAALGCLVKDYFPEPVTVSW |
| | region (CH1-CH2- | NSGALTSGVHTFPAVLQSSGLYSLSSVVTVPSSSLGTQTYI |
| | CH3) Hole | CNVNHKPSNTKVDKRVEPKSCDKTHTCPPCPAPELLGGPSV |
| | | FLFPPKPKDTLMISRTPEVTCVVVDVSHEDPEVKFNWYVDG |
| | | VEVHNAKTKPREEQYASTYRVVSVLTVLHQDWLNGKEYKCK |
| | | VSNKALPAPIEKTISKAKGQPREPQVCTLPPSREEMTKNQV |
| | | SLSCAVKGFYPSDIAVEWESNGQPENNYKTTPPVLDSDGSF |
| | | FLVSKLTVDKSRWQQGNVFSCSVMHEALHNHYTQKSLSLSP |
| SEQ ID NO: 3447 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | Hole | VSVLTVLHQDWLNGKEYKCKVSNKALPAPIEKTISKAKGQP |
| | | REPQVCTLPPSREEMTKNQVSLSCAVKGFYPSDIAVEWESN |
| | | GQPENNYKTTPPVLDSDGSFFLVSKLTVDKSRWQQGNVFSC |
| | | SVMHEALHNHYTQKSLSLSPGK |
| SEQ ID NO: 511 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | Hole | VSVLTVLHQDWLNGKEYKCKVSNKALPAPIEKTISKAKGQP |
| | | REPQVCTLPPSREEMTKNQVSLSCAVKGFYPSDIAVEWESN |
| | | GQPENNYKTTPPVLDSDGSFFLVSKLTVDKSRWQQGNVFSC |
| | | SVMHEALHNHYTQKSLSLSP |
| SEQ ID NO: 3644 | Light chain constant | RTVAAPSVFIFPPSDEQLKSGTASVVCLLNNFYPREAKVQW |
| | region | KVDNALQSGNSQESVTEQDSKDSTYSLSSTLTLSKADYEKH |
| | | KVYACEVTHQGLSSPVTKSFNRGEC |
| SEQ ID NO: 3472 | IL-15Rαsushi | ITCPPPMSVEHADIWVKSYSLYSRERYICNSGFKRKAGTSS |
| | (AA 31-108 of | LTECVLNKATNVAHWTTPSLKCIRDPALVHQRPAPPS |
| | IL-15Ralpha) | |
| SEQ ID NO: 3473 | Linker | GGSGGGGSGGGSGGGGSGGG |
| SEQ ID NO: 3673 | AA 49-162 of IL-15 | NWVNVISDLKKIEDLIQSMHIDATLYTESDVHPSCKVTAMK |
| | | CFLLELQVISLESGDASIHDTVEQLIILANNSLSSNGNVTE |
| | | SGCKECEELEEKNIKEFLQSFVHIVQMFINTS |
| SEQ ID NO: 8689 | IL-15Rαsushi-IL-15 | ITCPPPMSVEHADIWVKSYSLYSRERYICNSGFKRKAGTSS |
| | (AA 31-108 of IL- | LTECVLNKATNVAHWTTPSLKCIRDPALVHQRPAPPSGGSG |
| | 15Ralpha-Linker-AA | GGGSGGGSGGGGSGGGNWVNVISDLKKIEDLIQSMHIDATL |
| | 31-108 of IL- | YTESDVHPSCKVTAMKCFLLELQVISLESGDASIHDTVEQL |
| | 15Ralpha) | IILANNSLSSNGNVTESGCKECEELEEKNIKEFLQSFVHIV |
| | | QMFINTS |
| SEQ ID NO: 3308 | Linker | GGGGSGGGGS |
| SEQ ID NO: 3648 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | knob | VSVLTVLHQDWLNGKEYKCKVSNKALPAPIEKTISKAKGQP |
| | | REPQVYTLPPCREEMTKNQVSLWCLVKGFYPSDIAVEWESN |
| | | GQPENNYKTTPPVLDSDGSFFLYSKLTVDKSRWQQGNVFSC |
| | | SVMHEALHNHYTQKSLSLSPGK |
| SEQ ID NO: 508 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | knob | VSVLTVLHQDWLNGKEYKCKVSNKALPAPIEKTISKAKGQP |
| | | REPQVYTLPPCREEMTKNQVSLWCLVKGFYPSDIAVEWESN |
| | | GQPENNYKTTPPVLDSDGSFFLYSKLTVDKSRWQQGNVFSC |
| | | SVMHEALHNHYTQKSLSLSP |

BMM0360

| | | |
|---|---|---|
| SEQ ID NO: 4707 | Heavy chain (VH- | QVQLVQSGAEVKKPGSSVKVSCKASGHDFRLTYIHWVRQAP |
| | CH) - Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVCTLPPSREEMTKNQVSLSC |
| | | AVKGFYPSDIAVEWESNGQPENNYKTTPPVLDSDGSFFLVS |
| | | KLTVDKSRWQQGNVFSCSVMHEALHNHYTQKSLSLSPGK |
| SEQ ID NO: 3348 | Heavy chain (VH- | QVQLVQSGAEVKKPGSSVKVSCKASGHDFRLTYIHWVRQAP |
| | CH) - Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVCTLPPSREEMTKNQVSLSC |
| | | AVKGFYPSDIAVEWESNGQPENNYKTTPPVLDSDGSFFLVS |
| | | KLTVDKSRWQQGNVFSCSVMHEALHNHYTQKSLSLSP |
| SEQ ID NO: 3428 | Light chain (VL-CL) | DIQMTQSPSFLSASVGDRVTITCKASQNVADRVVWHQQKPG |
| | | KAPKALIYAASYLQSGVPSRFSGSGSGTEFTLTISSLQPED |
| | | FATYFCQQFKSYPLTFGQGTKLEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| SEQ ID NO: 3672 | IL-15Rαsushi-IL-15 | ITCPPPMSVEHADIWVKSYSLYSRERYICNSGFKRKAGTSS |
| | Heavy Chain Constant | LTECVLNKATNVAHWTTPSLKCIRDPALVHQRPAPPSGGSG |
| | Domain (Fc Region) - | GGGSGGGSGGGGSGGGNWVNVISDLKKIEDLIQSMHIDATL |
| | Knob | YTESDVHPSCKVTAMKCFLLELQVISLESGDASIHDTVEQL |
| | | IILANNSLSSNGNVTESGCKECEELEEKNIKEFLQSFVHIV |
| | | QMFINTSGGGGSGGGGSDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVYTLPPCREEMTKNQVSLWC |
| | | LVKGFYPSDIAVEWESNGQPENNYKTTPPVLDSDGSFFLYS |
| | | KLTVDKSRWQQGNVFSCSVMHEALHNHYTQKSLSLSPGK |
| SEQ ID NO: 4709 | IL-15Rαsushi-IL-15 | ITCPPPMSVEHADIWVKSYSLYSRERYICNSGFKRKAGTSS |
| | Heavy Chain Constant | LTECVLNKATNVAHWTTPSLKCIRDPALVHQRPAPPSGGSG |
| | Domain (Fc Region) - | GGGSGGGSGGGGSGGGNWVNVISDLKKIEDLIQSMHIDATL |
| | Knob | YTESDVHPSCKVTAMKCFLLELQVISLESGDASIHDTVEQL |
| | | IILANNSLSSNGNVTESGCKECEELEEKNIKEFLQSFVHIV |
| | | QMFINTSGGGGSGGGGSDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVYTLPPCREEMTKNQVSLWC |
| | | LVKGFYPSDIAVEWESNGQPENNYKTTPPVLDSDGSFFLYS |
| | | KLTVDKSRWQQGNVFSCSVMHEALHNHYTQKSLSLSP |
| SEQ ID NO: 3351 | HC CDR1 | GHDFRLTYIH |
| SEQ ID NO: 3421 | HC CDR1 | LTYIH |
| SEQ ID NO: 3702 | HC CDR1 | FRLTYIH |
| SEQ ID NO: 3352 | HC CDR2 | RVSAGSGNVKYNEKFKG |
| SEQ ID NO: 3353 | HC CDR3 | SYYSYDVLDY |
| SEQ ID NO: 3354 | LC CDR1 | KASQNVADRVV |
| SEQ ID NO: 3443 | LC CDR2 | AASYLQS |
| SEQ ID NO: 3356 | LC CDR3 | QQFKSYPLT |
| SEQ ID NO: 1346 | VH | QVQLVQSGAEVKKPGSSVKVSCKASGHDFRLTYIHWVRQAP |
| | | GQGLEWMGRVSAGSGNVKYNEKFKGRVTITADTSTSTAYME |
| | | LSSLRSEDTAVYYCAVSYYSYDVLDYWGQGTTVTVSS |
| SEQ ID NO: 3475 | VL | DIQMTQSPSFLSASVGDRVTITCKASQNVADRVVWHQQKPG |
| | | KAPKALIYAASYLQSGVPSRFSGSGSGTEFTLTISSLQPED |
| | | FATYFCQQFKSYPLTFGQGTKLEIK |
| SEQ ID NO: 3649 | Heavy chain constant | ASTKGPSVFPLAPSSKSTSGGTAALGCLVKDYFPEPVTVSW |
| | region (CH1-CH2- | NSGALTSGVHTFPAVLQSSGLYSLSSVVTVPSSSLGTQTYI |
| | CH3) Hole | CNVNHKPSNTKVDKRVEPKSCDKTHTCPPCPAPELLGGPSV |
| | | FLFPPKPKDTLMISRTPEVTCVVVDVSHEDPEVKFNWYVDG |
| | | VEVHNAKTKPREEQYASTYRVVSVLTVLHQDWLNGKEYKCK |
| | | VSNKALPAPIEKTISKAKGQPREPQVCTLPPSREEMTKNQV |
| | | SLSCAVKGFYPSDIAVEWESNGQPENNYKTTPPVLDSDGSF |
| | | FLVSKLTVDKSRWQQGNVFSCSVMHEALHNHYTQKSLSLSP |
| | | GK |
| SEQ ID NO: 3452 | Heavy chain constant | ASTKGPSVFPLAPSSKSTSGGTAALGCLVKDYFPEPVTVSW |
| | region (CH1-CH2- | NSGALTSGVHTFPAVLQSSGLYSLSSVVTVPSSSLGTQTYI |
| | CH3) Hole | CNVNHKPSNTKVDKRVEPKSCDKTHTCPPCPAPELLGGPSV |
| | | FLFPPKPKDTLMISRTPEVTCVVVDVSHEDPEVKFNWYVDG |
| | | VEVHNAKTKPREEQYASTYRVVSVLTVLHQDWLNGKEYKCK |
| | | VSNKALPAPIEKTISKAKGQPREPQVCTLPPSREEMTKNQV |
| | | SLSCAVKGFYPSDIAVEWESNGQPENNYKTTPPVLDSDGSF |
| | | FLVSKLTVDKSRWQQGNVFSCSVMHEALHNHYTQKSLSLSP |
| SEQ ID NO: 3447 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | Hole | VSVLTVLHQDWLNGKEYKCKVSNKALPAPIEKTISKAKGQP |
| | | REPQVCTLPPSREEMTKNQVSLSCAVKGFYPSDIAVEWESN |
| | | GQPENNYKTTPPVLDSDGSFFLVSKLTVDKSRWQQGNVFSC |
| | | SVMHEALHNHYTQKSLSLSPGK |
| SEQ ID NO: 511 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | Hole | VSVLTVLHQDWLNGKEYKCKVSNKALPAPIEKTISKAKGQP |
| | | REPQVCTLPPSREEMTKNQVSLSCAVKGFYPSDIAVEWESN |
| | | GQPENNYKTTPPVLDSDGSFFLVSKLTVDKSRWQQGNVFSC |
| | | SVMHEALHNHYTQKSLSLSP |
| SEQ ID NO: 3644 | Light chain constant | RTVAAPSVFIFPPSDEQLKSGTASVVCLLNNFYPREAKVQW |
| | region | KVDNALQSGNSQESVTEQDSKDSTYSLSSTLTLSKADYEKH |
| | | KVYACEVTHQGLSSPVTKSFNRGEC |
| SEQ ID NO: 3472 | IL-15Rαsushi | ITCPPPMSVEHADIWVKSYSLYSRERYICNSGFKRKAGTSS |
| | (AA 31-108 of | LTECVLNKATNVAHWTTPSLKCIRDPALVHQRPAPPS |
| | IL-15Ralpha) | |
| SEQ ID NO: 3473 | Linker | GGSGGGGSGGGSGGGGSGGG |
| SEQ ID NO: 3673 | AA 49-162 of IL-15 | NWVNVISDLKKIEDLIQSMHIDATLYTESDVHPSCKVTAMK |
| | | CFLLELQVISLESGDASIHDTVEQLIILANNSLSSNGNVTE |
| | | SGCKECEELEEKNIKEFLQSFVHIVQMFINTS |
| SEQ ID NO: 8689 | IL-15Rαsushi-IL-15 | ITCPPPMSVEHADIWVKSYSLYSRERYICNSGFKRKAGTSS |
| | (AA 31-108 of IL- | LTECVLNKATNVAHWTTPSLKCIRDPALVHQRPAPPSGGSG |
| | 15Ralpha-Linker-AA | GGGSGGGSGGGGSGGGNWVNVISDLKKIEDLIQSMHIDATL |
| | 31-108 of IL- | YTESDVHPSCKVTAMKCFLLELQVISLESGDASIHDTVEQL |
| | 15Ralpha) | IILANNSLSSNGNVTESGCKECEELEEKNIKEFLQSFVHIV |
| | | QMFINTS |
| SEQ ID NO: 3308 | Linker | GGGGSGGGGS |
| SEQ ID NO: 3648 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | knob | VSVLTVLHQDWLNGKEYKCKVSNKALPAPIEKTISKAKGQP |
| | | REPQVYTLPPCREEMTKNQVSLWCLVKGFYPSDIAVEWESN |
| | | GQPENNYKTTPPVLDSDGSFFLYSKLTVDKSRWQQGNVFSC |
| | | SVMHEALHNHYTQKSLSLSPGK |
| SEQ ID NO: 508 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | knob | VSVLTVLHQDWLNGKEYKCKVSNKALPAPIEKTISKAKGQP |
| | | REPQVYTLPPCREEMTKNQVSLWCLVKGFYPSDIAVEWESN |
| | | GQPENNYKTTPPVLDSDGSFFLYSKLTVDKSRWQQGNVFSC |
| | | SVMHEALHNHYTQKSLSLSP |

BMM0357

| | | |
|---|---|---|
| SEQ ID NO: 4707 | Heavy chain (VH- | QVQLVQSGAEVKKPGSSVKVSCKASGHDFRLTYIHWVRQAP |
| | CH) - Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVCTLPPSREEMTKNQVSLSC |
| | | AVKGFYPSDIAVEWESNGQPENNYKTTPPVLDSDGSFFLVS |
| | | KLTVDKSRWQQGNVFSCSVMHEALHNHYTQKSLSLSPGK |
| SEQ ID NO: 3348 | Heavy chain (VH- | QVQLVQSGAEVKKPGSSVKVSCKASGHDFRLTYIHWVRQAP |
| | CH) - Hole | GQGLEWMGRVSAGSGNVKYNEKFKGRVTITADTSTSTAYME |
| | | LSSLRSEDTAVYYCAVSYYSYDVLDYWGQGTTVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKRVEPKSCDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVCTLPPSREEMTKNQVSLSC |
| | | AVKGFYPSDIAVEWESNGQPENNYKTTPPVLDSDGSFFLVS |
| | | KLTVDKSRWQQGNVFSCSVMHEALHNHYTQKSLSLSP |
| SEQ ID NO: 3437 | Light chain (VL-CL) | DIQMTQSPSFLSASVGDRVTITCKASQNVADRVVWHQQKPG |
| | | KAPKALIFAASYLQSGVPSRFSGSGSGTEFTLTISSLQPED |
| | | FATYFCQQFKSYPLTFGQGTKLEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| SEQ ID NO: 3672 | IL-15Rαsushi-IL-15 | ITCPPPMSVEHADIWVKSYSLYSRERYICNSGFKRKAGTSS |
| | Heavy Chain Constant | LTECVLNKATNVAHWTTPSLKCIRDPALVHQRPAPPSGGSG |
| | Domain (Fc Region) - | GGGSGGGSGGGGSGGGNWVNVISDLKKIEDLIQSMHIDATL |
| | Knob | YTESDVHPSCKVTAMKCFLLELQVISLESGDASIHDTVEQL |
| | | IILANNSLSSNGNVTESGCKECEELEEKNIKEFLQSFVHIV |
| | | QMFINTSGGGGSGGGGSDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVYTLPPCREEMTKNQVSLWC |
| | | LVKGFYPSDIAVEWESNGQPENNYKTTPPVLDSDGSFFLYS |
| | | KLTVDKSRWQQGNVFSCSVMHEALHNHYTQKSLSLSPGK |
| SEQ ID NO: 4709 | IL-15Rαsushi-IL-15 | ITCPPPMSVEHADIWVKSYSLYSRERYICNSGFKRKAGTSS |
| | Heavy Chain Constant | LTECVLNKATNVAHWTTPSLKCIRDPALVHQRPAPPSGGSG |
| | Domain (Fc Region) - | GGGSGGGSGGGGSGGGNWVNVISDLKKIEDLIQSMHIDATL |
| | Knob | YTESDVHPSCKVTAMKCFLLELQVISLESGDASIHDTVEQL |
| | | IILANNSLSSNGNVTESGCKECEELEEKNIKEFLQSFVHIV |
| | | QMFINTSGGGGSGGGGSDKTHTCPPCPAPELLGGPSVFLFP |
| | | PKPKDTLMISRTPEVTCVVVDVSHEDPEVKFNWYVDGVEVH |
| | | NAKTKPREEQYASTYRVVSVLTVLHQDWLNGKEYKCKVSNK |
| | | ALPAPIEKTISKAKGQPREPQVYTLPPCREEMTKNQVSLWC |
| | | LVKGFYPSDIAVEWESNGQPENNYKTTPPVLDSDGSFFLYS |
| | | KLTVDKSRWQQGNVFSCSVMHEALHNHYTQKSLSLSP |
| SEQ ID NO: 3351 | HC CDR1 | GHDFRLTYIH |
| SEQ ID NO: 3421 | HC CDR1 | LTYIH |
| SEQ ID NO: 3702 | HC CDR1 | FRLTYIH |
| SEQ ID NO: 3352 | HC CDR2 | RVSAGSGNVKYNEKFKG |
| SEQ ID NO: 3353 | HC CDR3 | SYYSYDVLDY |
| SEQ ID NO: 3354 | LC CDR1 | KASQNVADRVV |
| SEQ ID NO: 3443 | LC CDR2 | AASYLQS |
| SEQ ID NO: 3356 | LC CDR3 | QQFKSYPLT |
| SEQ ID NO: 1346 | VH | QVQLVQSGAEVKKPGSSVKVSCKASGHDFRLTYIHWVRQAP |
| | | GQGLEWMGRVSAGSGNVKYNEKFKGRVTITADTSTSTAYME |
| | | LSSLRSEDTAVYYCAVSYYSYDVLDYWGQGTTVTVSS |
| SEQ ID NO: 3476 | VL | DIQMTQSPSFLSASVGDRVTITCKASQNVADRVVWHQQKPG |
| | | KAPKALIFAASYLQSGVPSRFSGSGSGTEFTLTISSLQPED |
| | | FATYFCQQFKSYPLTFGQGTKLEIK |
| SEQ ID NO: 3649 | Heavy chain constant | ASTKGPSVFPLAPSSKSTSGGTAALGCLVKDYFPEPVTVSW |
| | region (CH1-CH2- | NSGALTSGVHTFPAVLQSSGLYSLSSVVTVPSSSLGTQTYI |
| | CH3) Hole | CNVNHKPSNTKVDKRVEPKSCDKTHTCPPCPAPELLGGPSV |
| | | FLFPPKPKDTLMISRTPEVTCVVVDVSHEDPEVKFNWYVDG |
| | | VEVHNAKTKPREEQYASTYRVVSVLTVLHQDWLNGKEYKCK |
| | | VSNKALPAPIEKTISKAKGQPREPQVCTLPPSREEMTKNQV |
| | | SLSCAVKGFYPSDIAVEWESNGQPENNYKTTPPVLDSDGSF |
| | | FLVSKLTVDKSRWQQGNVFSCSVMHEALHNHYTQKSLSLSP |
| | | GK |
| SEQ ID NO: 3452 | Heavy chain constant | ASTKGPSVFPLAPSSKSTSGGTAALGCLVKDYFPEPVTVSW |
| | region (CH1-CH2- | NSGALTSGVHTFPAVLQSSGLYSLSSVVTVPSSSLGTQTYI |
| | CH3) Hole | CNVNHKPSNTKVDKRVEPKSCDKTHTCPPCPAPELLGGPSV |
| | | FLFPPKPKDTLMISRTPEVTCVVVDVSHEDPEVKFNWYVDG |
| | | VEVHNAKTKPREEQYASTYRVVSVLTVLHQDWLNGKEYKCK |
| | | VSNKALPAPIEKTISKAKGQPREPQVCTLPPSREEMTKNQV |
| | | SLSCAVKGFYPSDIAVEWESNGQPENNYKTTPPVLDSDGSF |
| | | FLVSKLTVDKSRWQQGNVFSCSVMHEALHNHYTQKSLSLSP |
| SEQ ID NO: 3447 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | Hole | VSVLTVLHQDWLNGKEYKCKVSNKALPAPIEKTISKAKGQP |
| | | REPQVCTLPPSREEMTKNQVSLSCAVKGFYPSDIAVEWESN |
| | | GQPENNYKTTPPVLDSDGSFFLVSKLTVDKSRWQQGNVFSC |
| | | SVMHEALHNHYTQKSLSLSPGK |
| SEQ ID NO: 511 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | Hole | VSVLTVLHQDWLNGKEYKCKVSNKALPAPIEKTISKAKGQP |
| | | REPQVCTLPPSREEMTKNQVSLSCAVKGFYPSDIAVEWESN |
| | | GQPENNYKTTPPVLDSDGSFFLVSKLTVDKSRWQQGNVFSC |
| | | SVMHEALHNHYTQKSLSLSP |
| SEQ ID NO: 3644 | Light chain constant | RTVAAPSVFIFPPSDEQLKSGTASVVCLLNNFYPREAKVQW |
| | region | KVDNALQSGNSQESVTEQDSKDSTYSLSSTLTLSKADYEKH |
| | | KVYACEVTHQGLSSPVTKSFNRGEC |
| SEQ ID NO: 3472 | IL-15Rαsushi | ITCPPPMSVEHADIWVKSYSLYSRERYICNSGFKRKAGTSS |
| | (AA 31-108 of | LTECVLNKATNVAHWTTPSLKCIRDPALVHQRPAPPS |
| | IL-15Ralpha) | |
| SEQ ID NO: 3473 | Linker | GGSGGGGSGGGSGGGGSGGG |
| SEQ ID NO: 3673 | AA 49-162 of IL-15 | NWVNVISDLKKIEDLIQSMHIDATLYTESDVHPSCKVTAMK |
| | | CFLLELQVISLESGDASIHDTVEQLIILANNSLSSNGNVTE |
| | | SGCKECEELEEKNIKEFLQSFVHIVQMFINTS |
| SEQ ID NO: 8689 | IL-15Rαsushi-IL-15 | ITCPPPMSVEHADIWVKSYSLYSRERYICNSGFKRKAGTSS |
| | (AA 31-108 of IL- | LTECVLNKATNVAHWTTPSLKCIRDPALVHQRPAPPSGGSG |
| | 15Ralpha-Linker-AA | GGGSGGGSGGGGSGGGNWVNVISDLKKIEDLIQSMHIDATL |
| | 31-108 of IL- | YTESDVHPSCKVTAMKCFLLELQVISLESGDASIHDTVEQL |
| | 15Ralpha) | IILANNSLSSNGNVTESGCKECEELEEKNIKEFLQSFVHIV |
| | | QMFINTS |
| SEQ ID NO: 3308 | Linker | GGGGGGGGS |
| SEQ ID NO: 3648 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | knob | VSVLTVLHQDWLNGKEYKCKVSNKALPAPIEKTISKAKGQP |
| | | REPQVYTLPPCREEMTKNQVSLWCLVKGFYPSDIAVEWESN |
| | | GQPENNYKTTPPVLDSDGSFFLYSKLTVDKSRWQQGNVFSC |
| | | SVMHEALHNHYTQKSLSLSPGK |
| SEQ ID NO: 508 | Heavy chain hinge - | DKTHTCPPCPAPELLGGPSVFLFPPKPKDTLMISRTPEVTC |
| | Fc region (CH2-CH3) | VVVDVSHEDPEVKFNWYVDGVEVHNAKTKPREEQYASTYRV |
| | knob | VSVLTVLHQDWLNGKEYKCKVSNKALPAPIEKTISKAKGQP |
| | | REPQVYTLPPCREEMTKNQVSLWCLVKGFYPSDIAVEWESN |
| | | GQPENNYKTTPPVLDSDGSFFLYSKLTVDKSRWQQGNVFSC |
| | | SVMHEALHNHYTQKSLSLSP |

TABLE 43
Amino acid sequences for anti TCRβ V5 antibodies. Amino acid and nucleotide
sequences for murine and humanized antibody molecules which bind to TCRVB 5
(e.g., TCRVB 5-5 or TCRVB 5-6). The amino acid of the heavy and light chain
CDRs, and the amino acid and nucleotide sequences of the heavy and light
chain variable regions, and the heavy and light chains are shown.
Murine antibody E, also referred to as MH3-2

| | | |
|---|---|---|
| SEQ ID NO: 1298 | HC CDR1 (Kabat) | SSWMN |
| SEQ ID NO: 1299 | HC CDR2 (Kabat) | RIYPGDGDTKYNGKFKG |
| SEQ ID NO: 1300 | HC CDR3 (Kabat) | RGTGGWYFDV |
| SEQ ID NO: 1302 | HC CDR1 (Chothia) | GYAFSSS |
| SEQ ID NO: 1303 | HC CDR2 (Chothia) | YPGDGD |
| SEQ ID NO: 1301 | HC CDR3 (Chothia) | RGTGGWYFDV |
| SEQ ID NO: 1304 | HC CDR1 (Combined) | GYAFSSSWMN |
| SEQ ID NO: 1299 | HC CDR2 (Combined)) | RIYPGDGDTKYNGKFKG |
| SEQ ID NO: 1301 | HC CDR3 (Combined) | RGTGGWYFDV |
| SEQ ID NO: 1305 | LC CDR1 (Kabat) | RASESVDSSGNSFMH |
| SEQ ID NO: 1306 | LC CDR2 (Kabat) | RASNLES |
| SEQ ID NO: 1307 | LC CDR3 (Kabat) | QQSFDDPFT |
| SEQ ID NO: 1308 | LC CDR1 (Chothia) | SESVDSSGNSF |
| SEQ ID NO: 1306 | LC CDR2 (Chothia) | RASNLES |
| SEQ ID NO: 1307 | LC CDR3 (Chothia) | QQSFDDPFT |
| SEQ ID NO: 1305 | LC CDR1 (Combined) | RASESVDSSGNSFMH |
| SEQ ID NO: 1306 | LC CDR2 (Combined) | RASNLES |
| SEQ ID NO: 1307 | LC CDR3 (Combined) | QQSFDDPFT |
| SEQ ID NO: 3091 | VH | QVQLQQSGPELVKPGASVKISCKASGYAFSSSWMNWV |
| | | KQRPGQGLEWIGRIYPGDGDTKYNGKFKGKATLTADK |
| | | SSSTAYMHLSSLTSVDSAVYFCARRGTGGWYFDVWGA |
| | | GTTVTVSS |
| SEQ ID NO: 3284 | Heavy chain | METDTLLLWVLLLWVPGSTGQVQLQQSGPELVKPGAS |
| | | VKISCKASGYAFSSSWMNWVKQRPGQGLEWIGRIYPG |
| | | DGDTKYNGKFKGKATLTADKSSSTAYMHLSSLTSVDS |
| | | AVYFCARRGTGGWYFDVWGAGTTVTVSSAKTTAPSVY |
| | | PLAPVCGDTTGSSVTLGCLVKGYFPEPVTLTWNSGSL |
| | | SSGVHTFPAVLQSDLYTLSSSVTVTSSTWPSQSITCN |
| | | VAHPASSTKVDKKIEPRGPTIKPCPPCKCPAPNLLGG |
| | | PSVFIFPPKIKDVLMISLSPIVTCVVVDVSEDDPDVQ |
| | | ISWFVNNVEVHTAQTQTHREDYNSTLRVVSALPIQHQ |
| | | DWMSGKEFKCKVNNKDLPAPIERTISKPKGSVRAPQV |
| | | YVLPPPEEEMTKKQVTLTCMVTDFMPEDIYVEWTNNG |
| | | KTELNYKNTEPVLDSDGSYFMYSKLRVEKKNWVERNS |
| | | YSCSVVHEGLHNHHTTKSFSRTPGK |
| SEQ ID NO: 3092 | VL | DIVLTQSPASLAVSLGQRATISCRASESVDSSGNSFM |
| | | HWYQQKPGQPPQLLIYRASNLESGIPARFSGSGSRTD |
| | | FTLTINPVEADDVATFYCQQSFDDPFTFGSGTKLEIK |
| SEQ ID NO: 3285 | Light chain | METDTLLLWVLLLWVPGSTGDIVLTQSPASLAVSLGQ |
| | | RATISCRASESVDSSGNSFMHWYQQKPGQPPQLLIYR |
| | | ASNLESGIPARFSGSGSRTDFTLTINPVEADDVATFY |
| | | CQQSFDDPFTFGSGTKLEIKRADAAPTVSIFPPSSEQ |
| | | LTSGGASVVCFLNNFYPKDINVKWKIDGSERQNGVLN |
| | | SWTDQDSKDSTYSMSSTLTLTKDEYERHNSYTCEATH |
| | | KTSTSPIVKSFNRNEC |

Humanized antibody E (E-H antibody)
Variable light chain (VL)

| | | | |
|---|---|---|---|
| SEQ ID NO: 3093 | VL | E-H.1 | DIVLTQSPDSLAVSLGERATINCRASESVDSSGNSFMHWYQQ |
| | | | KPGQPPQLLIYRASNLESGVPDRFSGSGSRTDFTLTISSLQA |
| | | | EDVAVYYCQQSFDDPFTFGQGTKLEIK |
| SEQ ID NO: 3094 | VL | E-H.2 | EIVLTQSPATLSLSPGERATLSCRASESVDSSGNSFMHWYQQ |
| | | | KPGQAPQLLIYRASNLESGIPARFSGSGSRTDFTLTISSLEP |
| | | | EDFAVYYCQQSFDDPFTFGQGTKLEIK |
| SEQ ID NO: 3095 | VL | E-H.3 | EIVLTQSPATLSLSPGERATLSCRASESVDSSGNSFMHWYQQ |
| | | | KPGQAPQLLIYRASNLESGIPARFSGSGSRTDFTLTISRLEP |
| | | | EDFAVYYCQQSFDDPFTFGQGTKLEIK |
| SEQ ID NO: 3096 | VL | E-H.4 | EIVLTQSPATLSLSPGERATLSCRASESVDSSGNSFMHWYQQ |
| | | | KPGQAPQLLIYRASNLESGIPARFSGSGSRTDFTLTISSLQP |
| | | | EDFAVYYCQQSFDDPFTFGQGTKLEIK |
| SEQ ID NO: 3097 | VL | E-H.5 | DIQLTQSPSSLSASVGDRVTITCRASESVDSSGNSFMHWYQQ |
| | | | KPGQAPQLLIYRASNLESGVPSRFSGSGSRTDFTLTISSLQP |
| | | | EDVATYYCQQSFDDPFTFGQGTKLEIK |
| SEQ ID NO: 3098 | VL | E-H.6 | EIVLTQSPATLSLSPGERATLSCRASESVDSSGNSFMHWYQQ |
| | | | KPGQAPQLLIYRASNLESGIPARFSGSGPRTDFTLTISSLEP |
| | | | EDFAVYYCQQSFDDPFTFGQGTKLEIK |
| SEQ ID NO: 3099 | VL | E-H.7 | EIVLTQSPATLSLSPGERATLSCRASESVDSSGNSFMHWYQQ |
| | | | KPGQAPQLLIYRASNLESGIPDRFSGSGSRTDFTLTISRLEP |
| | | | EDFAVYYCQQSFDDPFTFGQGTKLEIK |
| SEQ ID NO: 3100 | VL | E-H.8 | DIQLTQSPSSLSASVGDRVTITCRASESVDSSGNSFMHWYQQ |
| | | | KPGKVPQLLIYRASNLESGVPSRFSGSGSRTDFTLTISSLQP |
| | | | EDVATYYCQQSFDDPFTFGQGTKLEIK |
| SEQ ID NO: 3101 | VL | E-H.9 | DIQLTQSPSSLSASVGDRVTITCRASESVDSSGNSFMHWYQQ |
| | | | KPGKTPQLLIYRASNLESGIPSRFSGSGSRTDFTLTIRSLQP |
| | | | EDFATYYCQQSFDDPFTFGQGTKLEIK |
| SEQ ID NO: 3102 | VL | E-H.10 | EIVLTQSPGTLSLSPGERATLSCRASESVDSSGNSFMHWYQQ |
| | | | KPGQAPQLLIYRASNLESGIPDRFSGSGSRTDFTLTISRLEP |
| | | | EDFAVYYCQQSFDDPFTFGQGTKLEIK |
| SEQ ID NO: 3103 | VL | E-H.11 | EIVLTQSPATLSLSPGERATLSCRASESVDSSGNSFMHWYQQ |
| | | | KPGLAPQLLIYRASNLESGIPDRFSGSGSRTDFTLTISRLEP |
| | | | EDFAVYYCQQSFDDPFTFGQGTKLEIK |
| SEQ ID NO: 3104 | VL | E-H.12 | DIQLTQSPSSLSASVGDRVTITCRASESVDSSGNSFMHWYQQ |
| | | | KPGKAPQLLIYRASNLESGVPSRFSGSGSRTDFTLTISSLQP |
| | | | EDFATYYCQQSFDDPFTFGQGTKLEIK |
| SEQ ID NO: 3105 | VL | E-H.13 | DIQLTQSPSSVSASVGDRVTITCRASESVDSSGNSFMHWYQQ |
| | | | KPGKAPQLLIYRASNLESGVPSRFSGSGSRTDFTLTISSLQP |
| | | | EDFATYYCQQSFDDPFTFGQGTKLEIK |
| SEQ ID NO: 3106 | VL | E-H.14 | AIQLTQSPSSLSASVGDRVTITCRASESVDSSGNSFMHWYQQ |
| | | | KPGKAPQLLIYRASNLESGVPSRFSGSGSRTDFTLTISSLQP |
| | | | EDFATYYCQQSFDDPFTFGQGTKLEIK |
| SEQ ID NO: 3107 | VL | E-H.15 | DIQLTQSPSFLSASVGDRVTITCRASESVDSSGNSFMHWYQQ |
| | | | KPGKAPQLLIYRASNLESGVPSRFSGSGSRTEFTLTISSLQP |
| | | | EDFATYYCQQSFDDPFTFGQGTKLEIK |
| SEQ ID NO: 3108 | VL | E-H.16 | DIQLTQSPSSLSASVGDRVTITCRASESVDSSGNSFMHWYQQ |
| | | | KPGKAPQLLIYRASNLESGVPSRFSGSGSRTDFTFTISSLQP |
| | | | EDIATYYCQQSFDDPFTFGQGTKLEIK |
| SEQ ID NO: 3109 | VL | E-H.17 | EIVLTQSPATLSVSPGERATLSCRASESVDSSGNSFMHWYQQ |
| | | | KPGQAPQLLIYRASNLESGIPARFSGSGSRTEFTLTISILQS |
| | | | EDFAVYYCQQSFDDPFTFGQGTKLEIK |
| SEQ ID NO: 3110 | VL | E-H.18 | EIVLTQSPATLSVSPGERATLSCRASESVDSSGNSFMHWYQQ |
| | | | KPGQAPQLLIYRASNLESGIPARFSGSGSRTEFTLTISSLQS |
| | | | EDFAVYYCQQSFDDPFTFGQGTKLEIK |
| SEQ ID NO: 3111 | VL | E-H.19 | AIRLTQSPFSLSASVGDRVTITCRASESVDSSGNSFMHWYQQ |
| | | | KPAKAPQLFIYRASNLESGVPSRFSGSGSRTDFTLTISSLQP |
| | | | EDFATYYCQQSFDDPFTFGQGTKLEIK |
| SEQ ID NO: 3112 | VL | E-H.20 | DIQLTQSPSSLSASVGDRVTITCRASESVDSSGNSFMHWYQQ |
| | | | KPGKAPQSLIYRASNLESGVPSRFSGSGSRTDFTLTISSLQP |
| | | | EDFATYYCQQSFDDPFTFGQGTKLEIK |
| SEQ ID NO: 3113 | VL | E-H.21 | DIQLTQSPSSLSASVGDRVTITCRASESVDSSGNSFMHWYQQ |
| | | | KPGKAPQRLIYRASNLESGVPSRFSGSGSRTEFTLTISNLQP |
| | | | EDFATYYCQQSFDDPFTFGQGTKLEIK |
| SEQ ID NO: 3114 | VL | E-H.22 | DIQLTQSPSTLSASVGDRVTITCRASESVDSSGNSFMHWYQQ |
| | | | KPGKAPQLLIYRASNLESGVPSRFSGSGSRTEFTLTISSLQP |
| | | | DDFATYYCQQSFDDPFTFGQGTKLEIK |
| SEQ ID NO: 3115 | VL | E-H.23 | EIVLTQSPDFQSVTPKEKVTITCRASESVDSSGNSFMHWYQQ |
| | | | KPDQSPQLLIYRASNLESGVPSRFSGSGSRTDFTLTINSLEA |
| | | | EDAATYYCQQSFDDPFTFGQGTKLEIK |
| SEQ ID NO: 3116 | VL | E-H.24 | DIQLTQSPSSLSASVGDRVTITCRASESVDSSGNSFMHWYQQ |
| | | | KPGKAPQSLIYRASNLESGVPSKFSGSGSRTDFTLTISSLQP |
| | | | EDFATYYCQQSFDDPFTFGQGTKLEIK |
| SEQ ID NO: 3117 | VL | E-H.25 | DIQLTQSPSSLSASVGDRVTITCRASESVDSSGNSFMHWYQQ |
| | | | KPGKAPQRLIYRASNLESGVPSRFSGSGSRTEFTLTISSLQP |
| | | | EDFATYYCQQSFDDPFTFGQGTKLEIK |
| SEQ ID NO: 3118 | VL | E-H.26 | DIVLTQTPLSLSVTPGQPASISCRASESVDSSGNSFMHWYLQ |
| | | | KPGQPPQLLIYRASNLESGVPDRFSGSGSRTDFTLKISRVEA |
| | | | EDVGVYYCQQSFDDPFTFGQGTKLEIK |
| SEQ ID NO: 3119 | VL | E-H.27 | DIQLTQSPSSLSASVGDRVTITCRASESVDSSGNSFMHWYQQ |
| | | | KPEKAPQSLIYRASNLESGVPSRFSGSGSRTDFTLTISSLQP |
| | | | EDFATYYCQQSFDDPFTFGQGTKLEIK |
| SEQ ID NO: 3120 | VL | E-H.28 | EIVLTQSPPTLSLSPGERVTLSCRASESVDSSGNSFMHWYQQ |
| | | | KPGQAPQLLIYRASNLESGIPARFSGSGSRTDFTLTISSLQP |
| | | | EDFAVYYCQQSFDDPFTFGQGTKLEIK |
| SEQ ID NO: 3121 | VL | E-H.29 | DIQLTQSPSAMSASVGDRVTITCRASESVDSSGNSFMHWYQQ |
| | | | KPGKVPQRLIYRASNLESGVPSRFSGSGSRTEFTLTISSLQP |
| | | | EDFATYYCQQSFDDPFTFGQGTKLEIK |
| SEQ ID NO:3122 | VL | E-H.30 | DIVLTQSPLSLPVTPGEPASISCRASESVDSSGNSFMHWYLQ |
| | | | KPGQSPQLLIYRASNLESGVPDRFSGSGSRTDFTLKISRVEA |
| | | | EDVGVYYCQQSFDDPFTFGQGTKLEIK |
| SEQ ID NO: 3123 | VL | E-H.31 | DIVLTQTPLSLPVTPGEPASISCRASESVDSSGNSFMHWYLQ |
| | | | KPGQSPQLLIYRASNLESGVPDRFSGSGSRTDFTLKISRVEA |
| | | | EDVGVYYCQQSFDDPFTFGQGTKLEIK |
| SEQ ID NO: 3124 | VL | E-H.32 | DIVLTQTPLSLSVTPGQPASISCRASESVDSSGNSFMHWYLQ |
| | | | KPGQSPQLLIYRASNLESGVPDRFSGSGSRTDFTLKISRVEA |
| | | | EDVGVYYCQQSFDDPFTFGQGTKLEIK |
| SEQ ID NO: 3125 | VL | E-H.33 | EIVLTQSPPTLSLSPGERVTLSCRASESVDSSGNSFMHWYQQ |
| | | | KPGQAPQLLIYRASNLESSIPARFSGSGSRTDFTLTISSLQP |
| | | | EDFAVYYCQQSFDDPFTFGQGTKLEIK |
| SEQ ID NO: 3126 | VL | E-H.34 | DIVLTQSPLSLPVTLGQPASISCRASESVDSSGNSFMHWYQQ |
| | | | RPGQSPQRLIYRASNLESGVPDRFSGSGSRTDFTLKISRVEA |
| | | | EDVGVYYCQQSFDDPFTFGQGTKLEIK |
| SEQ ID NO: 3127 | VL | E-H.35 | DIVLTQTPLSSPVTLGQPASISCRASESVDSSGNSFMHWYQQ |
| | | | RPGQPPQLLIYRASNLESGVPDRFSGSGARTDFTLKISRVEA |
| | | | EDVGVYYCQQSFDDPFTFGQGTKLEIK |
| SEQ ID NO: 3128 | VL | E-H.36 | DIVLTQSPAFLSVTPGEKVTITCRASESVDSSGNSFMHWYQQ |
| | | | KPDQAPQLLIYRASNLESGVPSRFSGSGSRTDFTFTISSLEA |
| | | | EDAATYYCQQSFDDPFTFGQGTKLEIK |
| SEQ ID NO: 3129 | VL | E-H.37 | DIQLIQSPSFLSASVGDRVSIICRASESVDSSGNSFMHWYLQ |
| | | | KPGKSPQLFIYRASNLESGVSSRFSGRGSRTDFTLTIISLKP |
| | | | EDFAAYYCQQSFDDPFTFGQGTKLEIK |
| SEQ ID NO: 3130 | VL | E-H.38 | EIVLTQTPLSLSITPGEQASISCRASESVDSSGNSFMHWYLQ |
| | | | KARPVPQLLIYRASNLESGVPDRFSGSGSRTDFTLKISRVEA |
| | | | EDFGVYYCQQSFDDPFTFGQGTKLEIK |
| SEQ ID NO: 3131 | VL | E-H.39 | EIVLTQTPLSLSITPGEQASMSCRASESVDSSGNSFMHWYLQ |
| | | | KARPVPQLLIYRASNLESGVPDRFSGSGSRTDFTLKISRVEA |
| | | | EDFGVYYCQQSFDDPFTFGQGTKLEIK |
| SEQ ID NO: 3132 | VL | E-H.40 | EITLTQSPAFMSATPGDKVNISCRASESVDSSGNSFMHWYQQ |
| | | | KPGEAPQFIIYRASNLESGIPPRFSGSGYRTDFTLTINNIES |
| | | | EDAAYYYCQQSFDDPFTFGQGTKLEIK |

Variable HEAVY chain (VH)

| | | | |
|---|---|---|---|
| SEQ ID NO: 3133 | VH | E-H.1 | QVQLVQSGAEVKKPGASVKVSCKASGYAFSSSWMNWVRQAPG |
| | | | QGLEWIGRIYPGDGDTKYNGKFKGRATLTADKSTSTAYMELS |
| | | | SLRSEDTAVYYCARRGTGGWYFDVWGQGTTVTVSS |
| SEQ ID NO: 3134 | VH | E-H.2 | QVQLVQSGAEVKKPGSSVKVSCKASGYAFSSSWMNWVRQAPG |
| | | | QGLEWIGRIYPGDGDTKYNGKFKGRATLTADKSTSTAYMELS |
| | | | SLRSEDTAVYYCARRGTGGWYFDVWGQGTTVTVSS |
| SEQ ID NO: 3135 | VH | E-H.3 | QVQLVQSGAEVKKPGASVKVSCKASGYAFSSSWMNWVRQAPG |
| | | | KGLEWIGRIYPGDGDTKYNGKFKGRATLTADKSTSTAYMELS |
| | | | SLRSEDTAVYYCARRGTGGWYFDVWGQGTTVTVSS |
| SEQ ID NO: 3136 | VH | E-H.4 | QVQLVQSGAEVKKPGASVKVSCKASGYAFSSSWMNWVRQAPG |
| | | | QELEWIGRIYPGDGDTKYNGKFKGRATLTADKSISTAYMELS |
| | | | SLRSEDTATYYCARRGTGGWYFDVWGQGTTVTVSS |
| SEQ ID NO: 3137 | VH | E-H.5 | EVQLVQSGAEVKKPGATVKISCKASGYAFSSSWMNWVQQAPG |
| | | | KGLEWIGRIYPGDGDTKYNGKFKGRATLTADKSTSTAYMELS |
| | | | SLRSEDTAVYYCARRGTGGWYFDVWGQGTTVTVSS |
| SEQ ID NO: 3138 | VH | E-H.6 | QVQLVQSGAEVKKTGSSVKVSCKASGYAFSSSWMNWVRQAPG |
| | | | QALEWIGRIYPGDGDTKYNGKFKGRATLTADKSMSTAYMELS |
| | | | SLRSEDTAMYYCARRGTGGWYFDVWGQGTTVTVSS |
| SEQ ID NO: 3139 | VH | E-H.7 | QVQLVQSGAEVKKPGASVKVSCKASGYAFSSSWMNWVRQAPG |
| | | | QRLEWIGRIYPGDGDTKYNGKFKGRATLTADKSASTAYMELS |
| | | | SLRSEDMAVYYCARRGTGGWYFDVWGQGTTVTVSS |
| SEQ ID NO: 3140 | VH | E-H.8 | QVQLVQSGAEVKKPGASVKVSCKASGYAFSSSWMNWVRQAPG |
| | | | QGLEWIGRIYPGDGDTKYNGKFKGRATLTADKSTSTAYMELR |
| | | | SLRSDDMAVYYCARRGTGGWYFDVWGQGTTVTVSS |
| SEQ ID NO: 3141 | VH | E-H.9 | QVQLVQSGAEVKKPGASVKVSCKASGYAFSSSWMNWVRQAPG |
| | | | QRLEWIGRIYPGDGDTKYNGKFKGRATLTADKSASTAYMELS |
| | | | SLRSEDTAVYYCARRGTGGWYFDVWGQGTTVTVSS |
| SEQ ID NO: 3142 | VH | E-H.10 | QVQLVQSGAEVKKPGASVKVSCKASGYAFSSSWMNWVRQAPG |
| | | | QGLEWIGRIYPGDGDTKYNGKFKGRATLTADKSTSTAYMELR |
| | | | SLRSDDTAVYYCARRGTGGWYFDVWGQGTTVTVSS |
| SEQ ID NO: 3143 | VH | E-H.11 | QVQLVQSGAEVKKPGASVKVSCKASGYAFSSSWMNWVRQAPG |
| | | | QGLEWIGRIYPGDGDTKYNGKFKGRATLTADKSISTAYMELS |
| | | | RLRSDDTAVYYCARRGTGGWYFDVWGQGTTVTVSS |
| SEQ ID NO: 3144 | VH | E-H.12 | QVQLVQSGAEVKKPGASVKVSCKASGYAFSSSWMNWVRQAPG |
| | | | QGLEWIGRIYPGDGDTKYNGKFKGRATLTADKSISTAYMELS |
| | | | RLRSDDTVVYYCARRGTGGWYFDVWGQGTTVTVSS |
| SEQ ID NO: 3145 | VH | E-H.13 | QVQLVQSGAEVKKPGASVKVSCKASGYAFSSSWMNWVRQAPG |
| | | | QGLEWIGRIYPGDGDTKYNGKFKGWATLTADKSISTAYMELS |
| | | | RLRSDDTAVYYCARRGTGGWYFDVWGQGTTVTVSS |
| SEQ ID NO: 3146 | VH | E-H.14 | QVQLVQSGAEVKKPGASVKVSCKASGYAFSSSWMNWVRQATG |
| | | | QGLEWIGRIYPGDGDTKYNGKFKGRATLTANKSISTAYMELS |
| | | | SLRSEDTAVYYCARRGTGGWYFDVWGQGTTVTVSS |
| SEQ ID NO: 3147 | VH | E-H.15 | QVQLVQSGSELKKPGASVKVSCKASGYAFSSSWMNWVRQAPG |
| | | | QGLEWIGRIYPGDGDTKYNGKFKGRAVLSADKSVSTAYLQIS |
| | | | SLKAEDTAVYYCARRGTGGWYFDVWGQGTTVTVSS |
| SEQ ID NO: 3148 | VH | E-H.16 | QVQLVQSGPEVKKPGTSVKVSCKASGYAFSSSWMNWVRQARG |
| | | | QRLEWIGRIYPGDGDTKYNGKFKGRATLTADKSTSTAYMELS |
| | | | SLRSEDTAVYYCARRGTGGWYFDVWGQGTTVTVSS |
| SEQ ID NO: 3149 | VH | E-H.17 | EVQLVQSGAEVKKPGESLKISCKASGYAFSSSWMNWVRQMPG |
| | | | KGLEWIGRIYPGDGDTKYNGKFKGQATLSADKSISTAYLQWS |
| | | | SLKASDTAMYYCARRGTGGWYFDVWGQGTTVTVSS |
| SEQ ID NO: 3150 | VH | E-H.18 | QVQLVQSGSELKKPGASVKVSCKASGYAFSSSWMNWVRQAPG |
| | | | QGLEWIGRIYPGDGDTKYNGKFKGRAVLSADKSVSMAYLQIS |
| | | | SLKAEDTAVYYCARRGTGGWYFDVWGQGTTVTVSS |
| SEQ ID NO: 3151 | VH | E-H.19 | QVQLVQSGHEVKQPGASVKVSCKASGYAFSSSWMNWVPQAPG |
| | | | QGLEWIGRIYPGDGDTKYNGKFKGRAVLSADKSASTAYLQIS |
| | | | SLKAEDMAMYYCARRGTGGWYFDVWGQGTTVTVSS |
| SEQ ID NO: 3152 | VH | E-H.20 | EVQLVQSGAEVKKPGESLKISCKASGYAFSSSWMNWVRQMPG |
| | | | KGLEWIGRIYPGDGDTKYNGKFKGQATLSADKPISTAYLQWS |
| | | | SLKASDTAMYYCARRGTGGWYFDVWGQGTTVTVSS |
| SEQ ID NO: 3153 | VH | E-H.21 | EVQLVQSGAEVKKPGESLRISCKASGYAFSSSWMNWVRQMPG |
| | | | KGLEWIGRIYPGDGDTKYNGKFKGQATLSADKSISTAYLQWS |
| | | | SLKASDTAMYYCARRGTGGWYFDVWGQGTTVTVSS |
| SEQ ID NO: 3154 | VH | E-H.22 | EVQLVQSGAEVKKPGESLRISCKASGYAFSSSWMNWVRQMPG |
| | | | KGLEWIGRIYPGDGDTKYNGKFKGHATLSADKSISTAYLQWS |
| | | | SLKASDTAMYYCARRGTGGWYFDVWGQGTTVTVSS |
| SEQ ID NO: 3155 | VH | E-H.23 | QVQLVQSGAEVKKTGSSVKVSCKASGYAFSSSWMNWVRQAPR |
| | | | QALEWIGRIYPGDGDTKYNGKFKGRATLTADKSMSTAYMELS |
| | | | SLRSEDTAMYYCARRGTGGWYFDVWGQGTTVTVSS |
| SEQ ID NO: 3156 | VH | E-H.24 | EVQLVESGGGLVQPGRSLRLSCTASGYAFSSSWMNWVRQAPG |
| | | | KGLEWIGRIYPGDGDTKYNGKFKGRATLSADKSKSIAYLQMN |
| | | | SLKTEDTAVYYCARRGTGGWYFDVWGQGTTVTVSS |
| SEQ ID NO: 3157 | VH | E-H.25 | EVQLVESGGGLVQPGPSLRLSCTASGYAFSSSWMNWVRQAPG |
| | | | KGLEWIGRIYPGDGDTKYNGKFKGRATLSADKSKSIAYLQMN |
| | | | SLKTEDTAVYYCARRGTGGWYFDVWGQGTTVTVSS |
| SEQ ID NO: 3158 | VH | E-H.26 | QVQLQESGPGLVKPSQTLSLTCTASGYAFSSSWMNWVRQPPG |
| | | | KGLEWIGRIYPGDGDTKYNGKFKGRATLSADKSKSQASLKLS |
| | | | SVTAADTAVYYCARRGTGGWYFDVWGQGTTVTVSS |
| SEQ ID NO: 3159 | VH | E-H.27 | QVQLQESGPGLVKPSGTLSLTCAASGYAFSSSWMNWVRQPPG |
| | | | KGLEWIGRIYPGDGDTKYNGKFKGRATLSADKSKSQASLKLS |
| | | | SVTAADTAVYYCARRGTGGWYFDVWGQGTTVTVSS |
| SEQ ID NO: 3160 | VH | E-H.28 | EVQLVESGGGLVKPGRSLRLSCTASGYAFSSSWMNWVRQAPG |
| | | | KGLEWIGRIYPGDGDTKYNGKFKGRATLSADKSKSIAYLQMN |
| | | | SLKTEDTAVYYCARRGTGGWYFDVWGQGTTVTVSS |
| SEQ ID NO: 3161 | VH | E-H.29 | EVQLVESGGGLVQPGGSLKLSCAASGYAFSSSWMNWVRQASG |
| | | | KGLEWIGRIYPGDGDTKYNGKFKGRATLSADKSKSTAYLQMN |
| | | | SLKTEDTAVYYCARRGTGGWYFDVWGQGTTVTVSS |
| SEQ ID NO: 3162 | VH | E-H.30 | QVQLQESGPGLVKPSQTLSLTCAASGYAFSSSWMNWVRQPPG |
| | | | KGLEWIGRIYPGDGDTKYNGKFKGRATLSADKSKSQASLKLS |
| | | | SVTAADTAVYYCARRGTGGWYFDVWGQGTTVTVSS |
| SEQ ID NO: 3163 | VH | E-H.31 | EVQLVESGGGLVKPGGSLRLSCAASGYAFSSSWMNWVRQAPG |
| | | | KGLEWIGRIYPGDGDTKYNGKFKGRATLSADKSKSTAYLQMN |
| | | | SLKTEDTAVYYCARRGTGGWYFDVWGQGTTVTVSS |
| SEQ ID NO: 3164 | VH | E-H.32 | EVQLVESGGALVKPGGSLRLSCAASGYAFSSSWMNWVRQAPG |
| | | | KGLEWIGRIYPGDGDTKYNGKFKGRATLSADKSKSTAYLQMN |
| | | | SLKTEDTAVYYCARRGTGGWYFDVWGQGTTVTVSS |
| SEQ ID NO: 3165 | VH | E-H.33 | QVQLQESGPGLVKPSQTLSLTCAAYGYAFSSSWMNWVRQPPG |
| | | | KGLEWIGRIYPGDGDTKYNGKFKGRATLSADKSKSQASLKLS |
| | | | SVTAADTAVYYCARRGTGGWYFDVWGQGTTVTVSS |
| SEQ ID NO: 3166 | VH | E-H.34 | QVQLQESGSGLVKPSQTLSLTCAASGYAFSSSWMNWVRQPPG |
| | | | KGLEWIGRIYPGDGDTKYNGKFKGRATLSADKSKSQASLKLS |
| | | | SVTAADTAVYYCARRGTGGWYFDVWGQGTTVTVSS |
| SEQ ID NO: 3167 | VH | E-H.35 | EVQLVESGGGLVQPGGSLRLSCAASGYAFSSSWMNWVRQAPG |
| | | | KGLEWIGRIYPGDGDTKYNGKFKGRATLSADKSKSSAYLQMN |
| | | | SLKTEDTAVYYCARRGTGGWYFDVWGQGTTVTVSS |
| SEQ ID NO: 3168 | VH | E-H.36 | QVQLQESGPGLVKPSDTLSLTCTASGYAFSSSWMNWVRQPPG |
| | | | KGLEWIGRIYPGDGDTKYNGKFKGRATLSADKSKSQASLKLS |
| | | | SVTAADTAVYYCARRGTGGWYFDVWGQGTTVTVSS |
| SEQ ID NO: 3169 | VH | E-H.37 | QVQLQESGPGLVKPSQTLSLTCTASGYAFSSSWMNWVRQHPG |
| | | | KGLEWIGRIYPGDGDTKYNGKFKGRATLSADKSKSQASLKLS |
| | | | SVTAADTAVYYCARRGTGGWYFDVWGQGTTVTVSS |
| SEQ ID NO: 3170 | VH | E-H.38 | QVQLQESGPGLVKPSQTLSLTCTASGYAFSSSWMNWVRQHPG |
| | | | KGLEWIGRIYPGDGDTKYNGKFKGLATLSADKSKSQASLKLS |
| | | | SVTAADTAVYYCARRGTGGWYFDVWGQGTTVTVSS |
| SEQ ID NO: 3171 | VH | E-H.39 | QVQLVESGGGVVQPGRSLRLSCAASGYAFSSSWMNWVRQAPG |
| | | | KGLEWIGRIYPGDGDTKYNGKFKGRATLSADKSKSTAYLQMS |
| | | | SLRAEDTAVYYCARRGTGGWYFDVWGQGTTVTVSS |
| SEQ ID NO: 3172 | VH | E-H.40 | QVQLVESGGGLVKPGGSLRLSCAASGYAFSSSWMNWVRQAPG |
| | | | KGLEWIGRIYPGDGDTKYNGKFKGRATLSADKAKSSAYLQMN |
| | | | SLRAEDTAVYYCARRGTGGWYFDVWGQGTTVTVSS |
| SEQ ID NO: 3173 | VH | E-H.41 | QVQLVESGGGLVQPGGSLRLSCSASGYAFSSSWMNWVRQAPG |
| | | | KGLEWIGRIYPGDGDTKYNGKFKGRATLSADKSKSTAYLQMN |
| | | | SLRAEDTAVYYCARRGTGGWYFDVWGQGTTVTVSS |
| SEQ ID NO: 3174 | VH | E-H.42 | QVQLLESGGGLVKPGGSLRLSCAASGYAFSSSWMNWVRQAPG |
| | | | KGLEWIGRIYPGDGDTKYNGKFKGRATLSADKAKSSAYLQMN |
| | | | SLRAEDTAVYYCARRGTGGWYFDVWGQGTTVTVSS |
| SEQ ID NO: 3175 | VH | E-H.43 | EVQLVESGGGLVQPGGSLRLSCSASGYAFSSSWMNWVRQAPG |
| | | | KGLEWIGRIYPGDGDTKYNGKFKGRATLSADKSKSTAYLQMS |
| | | | SLRAEDTAVYYCARRGTGGWYFDVWGQGTTVTVSS |
| SEQ ID NO: 3176 | VH | E-H.44 | QVQLQESGPGLVKPSDTLSLTCAASGYAFSSSWMNWVRQPPG |
| | | | KGLEWIGRIYPGDGDTKYNGKFKGRATLSADKSKSQASLKLS |
| | | | SVTAVDTAVYYCARRGTGGWYFDVWGQGTTVTVSS |
| SEQ ID NO: 3177 | VH | E-H.45 | QVQLQESGPGLVKPSQTLSLTCAASGYAFSSSWMNWVRQPPG |
| | | | KGLEWIGRIYPGDGDTKYNGKFKGRATLSADKSKSQASLKLS |
| | | | SVTAVDTAVYYCARRGTGGWYFDVWGQGTTVTVSS |
| SEQ ID NO: 3178 | VH | E-H.46 | EVQLVESGGGLVQPGGSLRLSCSASGYAFSSSWMNWVRQAPG |
| | | | KGLEWIGRIYPGDGDTKYNGKFKGRATLSADKSKSTAYVQMS |
| | | | SLRAEDTAVYYCARRGTGGWYFDVWGQGTTVTVSS |
| SEQ ID NO: 3179 | VH | E-H.47 | QVQLVDSGGGVVQPGRSLRLSCAASGYAFSSSWMNWVRQAPG |
| | | | KGLEWIGRIYPGDGDTKYNGKFKGRATLSADKSKSTAYLQMN |
| | | | SLRAEDTAVYYCARRGTGGWYFDVWGQGTTVTVSS |
| SEQ ID NO: 3180 | VH | E-H.48 | QVQLVESGGGVVQPGRSLRLSCAASGYAFSSSWMNWVRQAPG |
| | | | KGLEWIGRIYPGDGDTKYNGKFKGRATLSADKSKSTAYLQMN |
| | | | SLRAEGTAVYYCARRGTGGWYFDVWGQGTTVTVSS |
| SEQ ID NO: 3181 | VH | E-H.49 | QVQLVESGGGVVQPGRSLRLSCAASGYAFSSSWMNWVRQAPG |
| | | | KGLEWIGRIYPGDGDTKYNGKFKGRATLSADKSKSTAYLQMN |
| | | | SLRAEDTAVYYCARRGTGGWYFDVWGQGTTVTVSS |
| SEQ ID NO: 3182 | VH | E-H.50 | EVQLVESGGGLVQPGGSLRLSCAASGYAFSSSWMNWVRQAPG |
| | | | KGLEWIGRIYPGDGDTKYNGKFKGRATLSADKSKSTAYLQMN |
| | | | SLRAEDTAVYYCARRGTGGWYFDVWGQGTTVTVSS |

TABLE 44
Amino acid sequences for anti TCRβ V10 antibodies. Amino acid and nucleotide
sequences for murine and humanized antibody molecules which bind to TCRBV 10 (e.g., TCRBV
10-1, TCRBV 10-2 or TCRBV 10-3). The amino acid of the heavy and light chain CDRs, and the
amino acid and nucleotide sequences of the heavy and light chain variable regions, and
the heavy and light chains are shown.
Murine antibody D, also referred to as S511 antibody

| | | |
|---|---|---|
| SEQ ID NO: 1288 | HC CDR1 (Kabat) | SYGMS |
| SEQ ID NO: 1289 | HC CDR2 (Kabat) | LISSGGSYTYYTDSVKG |
| SEQ ID NO: 1290 | HC CDR3 (Kabat) | HGGNFFDY |
| SEQ ID NO: 1291 | HC CDR1 (Chothia) | GFTFRSY |
| SEQ ID NO: 1292 | HC CDR2 (Chothia) | SSGGSY |
| SEQ ID NO: 1290 | HC CDR3 (Chothia) | HGGNFFDY |
| SEQ ID NO: 1293 | HC CDR1 (Combined) | GFTFRSYGMS |
| SEQ ID NO: 1289 | HC CDR2 (Combined)) | LISSGGSYTYYTDSVKG |
| SEQ ID NO: 1290 | HC CDR3(Combined) | HGGNFFDY |
| SEQ ID NO: 1294 | LC CDR1 (Kabat) | SVSSSVSYMH |
| SEQ ID NO: 1295 | LC CDR2 (Kabat) | DTSKLAS |
| SEQ ID NO: 1296 | LC CDR3 (Kabat) | QQWSSNPQYT |
| SEQ ID NO: 1297 | LC CDR1 (Chothia) | SSSVSY |
| SEQ ID NO: 1295 | LC CDR2 (Chothia) | DTSKLAS |
| SEQ ID NO: 1296 | LC CDR3 (Chothia) | QQWSSNPQYT |
| SEQ ID NO: 1294 | LC CDR1 (Combined) | SVSSSVSYMH |
| SEQ ID NO: 1295 | LC CDR2 (Combined) | DTSKLAS |
| SEQ ID NO: 1296 | LC CDR3 (Combined) | QQWSSNPQYT |
| SEQ ID NO: 3183 | VH | EVQLVESGGDLVKPGGSLKLSCAVSGFTFRSYGMSWVR |
| | | QTPDKRLEWVALISSGGSYTYYTDSVKGRFTISRDNAKN |
| | | TLYLQMSSLKSEDTAIYYCSRHGGNFFDYWGQGTTLTVS |
| | | S |
| SEQ ID NO: 3184 | VL | QIVLTQSPSIMSASPGEKVTMTCSVSSSVSYMHWYQQKS |
| | | GTSPKRWIYDTSKLASGVPARFSGSGSGTSYSLTISSMEA |
| | | EDAATYYCQQWSSNPQYTFGGGTKLEIK |

Humanized antibody D (D-H antibody)
Variable light chain (VL)

| | | | |
|---|---|---|---|
| SEQ ID NO: 3185 | VL | D-H.1 | DIVLTQSPAFLSVTPGEKVTITCSVSSSVSYMHWYQQKPDQAPK |
| | | | LLIYDTSKLASGVPSRFSGSGSGTDYTFTISSLEAEDAATYYCQQ |
| | | | WSSNPQYTFGQGTKLEIK |
| SEQ ID NO: 3186 | VL | D-H.2 | AIQLTQSPSSLSASVGDRVTITCSVSSSVSYMHWYQQKPGKAPK |
| | | | LLIYDTSKLASGVPSRFSGSGSGTDYTLTISSLQPEDFATYYCQQ |
| | | | WSSNPQYTFGQGTKLEIK |
| SEQ ID NO: 3187 | VL | D-H.3 | DIQLTQSPSFLSASVGDRVTITCSVSSSVSYMHWYQQKPGKAPK |
| | | | LLIYDTSKLASGVPSRFSGSGSGTEYTLTISSLQPEDFATYYCQQ |
| | | | WSSNPQYTFGQGTKLEIK |
| SEQ ID NO: 3188 | VL | D-H.4 | DIQLTQSPSSLSASVGDRVTITCSVSSSVSYMHWYQQKPGKAPK |
| | | | LLIYDTSKLASGVPSRFSGSGSGTDYTLTISSLQPEDFATYYCQQ |
| | | | WSSNPQYTFGQGTKLEIK |
| SEQ ID NO: 3189 | VL | D-H.5 | DIQLTQSPSSVSASVGDRVTITCSVSSSVSYMHWYQQKPGKAPK |
| | | | LLIYDTSKLASGVPSRFSGSGSGTDYTLTISSLQPEDFATYYCQQ |
| | | | WSSNPQYTFGQGTKLEIK |
| SEQ ID NO: 3190 | VL | D-H.6 | DIQLTQSPSSLSASVGDRVTITCSVSSSVSYMHWYQQKPGKVPK |
| | | | LLIYDTSKLASGVPSRFSGSGSGTDYTLTISSLQPEDVATYYCQQ |
| | | | WSSNPQYTFGQGTKLEIK |
| SEQ ID NO: 3191 | VL | D-H.7 | DIQLTQSPSSLSASVGDRVTITCSVSSSVSYMHWYQQKPGQAPK |
| | | | LLIYDTSKLASGVPSRFSGSGSGTDYTLTISSLQPEDVATYYCQQ |
| | | | WSSNPQYTFGQGTKLEIK |
| SEQ ID NO: 3192 | VL | D-H.8 | EIVLTQSPDFQSVTPKEKVTITCSVSSSVSYMHWYQQKPDQSPKL |
| | | | LIYDTSKLASGVPSRFSGSGSGTDYTLTINSLEAEDAATYYCQQ |
| | | | WSSNPQYTFGQGTKLEIK |
| SEQ ID NO: 3193 | VL | D-H.9 | AIRLTQSPFSLSASVGDRVTITCSVSSSVSYMHWYQQKPAKAPK |
| | | | LFIYDTSKLASGVPSRFSGSGSGTDYTLTISSLQPEDFATYYCQQ |
| | | | WSSNPQYTFGQGTKLEIK |
| SEQ ID NO: 3194 | VL | D- | DIQLTQSPSSLSASVGDRVTITCSVSSSVSYMHWYQQKPGKAPK |
| | | H.10 | LLIYDTSKLASGVPSRFSGSGSGTDYTFTISSLQPEDIATYYCQQ |
| | | | WSSNPQYTFGQGTKLEIK |
| SEQ ID NO: 3195 | VL | D- | EIVLTQSPATLSLSPGERATLSCSVSSSVSYMHWYQQKPGQAPK |
| | | H.11 | LLIYDTSKLASGIPARFSGSGSGTDYTLTISSLEPEDFAVYYCQQ |
| | | | WSSNPQYTFGQGTKLEIK |
| SEQ ID NO: 3196 | VL | D- | DIQLTQSPSTLSASVGDRVTITCSVSSSVSYMHWYQQKPGKAPK |
| | | H.12 | LLIYDTSKLASGVPSRFSGSGSGTEYTLTISSLQPDDFATYYCQQ |
| | | | WSSNPQYTFGQGTKLEIK |
| SEQ ID NO: 3197 | VL | D- | DIQLTQSPSSLSASVGDRVTITCSVSSSVSYMHWYQQKPGKTPK |
| | | H.13 | LLIYDTSKLASGIPSRFSGSGSGTDYTLTIRSLQPEDFATYYCQQ |
| | | | WSSNPQYTFGQGTKLEIK |
| SEQ ID NO: 3198 | VL | D- | EIVLTQSPPTLSLSPGERVTLSCSVSSSVSYMHWYQQKPGQAPKL |
| | | H.14 | LIYDTSKLASGIPARFSGSGSGTDYTLTISSLQPEDFAVYYCQQW |
| | | | SSNPQYTFGQGTKLEIK |
| SEQ ID NO: 3199 | VL | D- | DIQLTQSPSSLSASVGDRVTITCSVSSSVSYMHWYQQKPGKAPK |
| | | H.15 | RLIYDTSKLASGVPSRFSGSGSGTEYTLTISSLQPEDFATYYCQQ |
| | | | WSSNPQYTFGQGTKLEIK |
| SEQ ID NO: 3200 | VL | D- | EIVLTQSPATLSLSPGERATLSCSVSSSVSYMHWYQQKPGQAPK |
| | | H.16 | LLIYDTSKLASGIPARFSGSGPGTDYTLTISSLEPEDFAVYYCQQ |
| | | | WSSNPQYTFGQGTKLEIK |
| SEQ ID NO: 3201 | VL | D- | EIVLTQSPATLSLSPGERATLSCSVSSSVSYMHWYQQKPGQAPK |
| | | H.17 | LLIYDTSKLASGIPARFSGSGSGTDYTLTISRLEPEDFAVYYCQQ |
| | | | WSSNPQYTFGQGTKLEIK |
| SEQ ID NO: 3202 | VL | D- | EIVLTQSPATLSLSPGERATLSCSVSSSVSYMHWYQQKPGQAPK |
| | | H.18 | LLIYDTSKLASGIPARFSGSGSGTDYTLTISSLQPEDFAVYYCQQ |
| | | | WSSNPQYTFGQGTKLEIK |
| SEQ ID NO: 3203 | VL | D- | EIVLTQSPATLSVSPGERATLSCSVSSSVSYMHWYQQKPGQAPK |
| | | H.19 | LLIYDTSKLASGIPARFSGSGSGTEYTLTISSLQSEDFAVYYCQQ |
| | | | WSSNPQYTFGQGTKLEIK |
| SEQ ID NO: 3204 | VL | D- | EIVLTQSPATLSVSPGERATLSCSVSSSVSYMHWYQQKPGQAPK |
| | | H.20 | LLIYDTSKLASGIPARFSGSGSGTEYTLTISILQSEDFAVYYCQQW |
| | | | SSNPQYTFGQGTKLEIK |
| SEQ ID NO: 3205 | VL | D- | EIVLTQSPPTLSLSPGERVTLSCSVSSSVSYMHWYQQKPGQAPKL |
| | | H.21 | LIYDTSKLASSIPARFSGSGSGTDYTLTISSLQPEDFAVYYCQQWS |
| | | | SNPQYTFGQGTKLEIK |
| SEQ ID NO: 3206 | VL | D- | DIQLTQSPSSLSASVGDRVTITCSVSSSVSYMHWYQQKPGKAPK |
| | | H.22 | SLIYDTSKLASGVPSRFSGSGSGTDYTLTISSLQPEDFATYYCQQ |
| | | | WSSNPQYTFGQGTKLEIK |
| SEQ ID NO: 3207 | VL | D- | DIQLTQSPSSLSASVGDRVTITCSVSSSVSYMHWYQQKPGKAPK |
| | | H.23 | RLIYDTSKLASGVPSRFSGSGSGTEYTLTISNLQPEDFATYYCQQ |
| | | | WSSNPQYTFGQGTKLEIK |
| SEQ ID NO: 3208 | VL | D- | DIQLTQSPSAMSASVGDRVTITCSVSSSVSYMHWYQQKPGK VPK |
| | | H.24 | RLIYDTSKLASGVPSRFSGSGSGTEYTLTISSLQPEDFATYYCQQ |
| | | | WSSNPQYTFGQGTKLEIK |
| SEQ ID NO: 3209 | VL | D- | EIVLTQSPATLSLSPGERATLSCSVSSSVSYMHWYQQKPGQAPK |
| | | H.25 | LLIYDTSKLASGIPDRFSGSGSGTDYTLTISRLEPEDFAVYYCQQ |
| | | | WSSNPQYTFGQGTKLEIK |
| SEQ ID NO: 3210 | VL | D- | EIVLTQSPATLSLSPGERATLSCSVSSSVSYMHWYQQKPGLAPKL |
| | | H.26 | LIYDTSKLASGIPDRFSGSGSGTDYTLTISRLEPEDFAVYYCQQW |
| | | | SSNPQYTFGQGTKLEIK |
| SEQ ID NO: 3211 | VL | D- | EIVLTQSPGTLSLSPGERATLSCSVSSSVSYMHWYQQKPGQAPK |
| | | H.27 | LLIYDTSKLASGIPDRFSGSGSGTDYTLTISRLEPEDFAVYYCQQ |
| | | | WSSNPQYTFGQGTKLEIK |
| SEQ ID NO: 3212 | VL | D- | DIQLTQSPSSLSASVGDRVTITCSVSSSVSYMHWYQQKPGKAPK |
| | | H.28 | SLIYDTSKLASGVPSKFSGSGSGTDYTLTISSLQPEDFATYYCQQ |
| | | | WSSNPQYTFGQGTKLEIK |
| SEQ ID NO: 3213 | VL | D- | DIQLTQSPSSLSASVGDRVTITCSVSSSVSYMHWYQQKPEKAPKS |
| | | H.29 | LIYDTSKLASGVPSRFSGSGSGTDYTLTISSLQPEDFATYYCQQW |
| | | | SSNPQYTFGQGTKLEIK |
| SEQ ID NO: 3214 | VL | D- | DIVLTQSPDSLAVSLGERATINCSVSSSVSYMHWYQQKPGQPPK |
| | | H.30 | LLIYDTSKLASGVPDRFSGSGSGTDYTLTISSLQAEDVAVYYCQQ |
| | | | WSSNPQYTFGQGTKLEIK |
| SEQ ID NO: 3215 | VL | D- | EIVLTQTPLSLSITPGEQASMSCSVSSSVSYMHWYLQKARPVPKL |
| | | H.31 | LIYDTSKLASGVPDRFSGSGSGTDYTLKISRVEAEDFGVYYCQQ |
| | | | WSSNPQYTFGQGTKLEIK |
| SEQ ID NO: 3216 | VL | D- | EIVLTQTPLSLSITPGEQASISCSVSSSVSYMHWYLQKARPVPKLL |
| | | H.32 | IYDTSKLASGVPDRFSGSGSGTDYTLKISRVEAEDFGVYYCQQW |
| | | | SSNPQYTFGQGTKLEIK |
| SEQ ID NO: 3217 | VL | D- | DIVLTQSPLSLPVTPGEPASISCSVSSSVSYMHWYLQKPGQSPKL |
| | | H.33 | LIYDTSKLASGVPDRFSGSGSGTDYTLKISRVEAEDVGVYYCQQ |
| | | | WSSNPQYTFGQGTKLEIK |
| SEQ ID NO: 3218 | VL | D- | DIVLTQSPLSLPVTLGQPASISCSVSSSVSYMHWYQQRPGQSPKR |
| | | H.34 | LIYDTSKLASGVPDRFSGSGSGTDYTLKISRVEAEDVGVYYCQQ |
| | | | WSSNPQYTFGQGTKLEIK |
| SEQ ID NO: 3219 | VL | D- | DIVLTQTPLSLPVTPGEPASISCSVSSSVSYMHWYLQKPGQSPKL |
| | | H.35 | LIYDTSKLASGVPDRFSGSGSGTDYTLKISRVEAEDVGVYYCQQ |
| | | | WSSNPQYTFGQGTKLEIK |
| SEQ ID NO: 3220 | VL | D- | DIVLTQTPLSLSVTPGQPASISCSVSSSVSYMHWYLQKPGQSPKL |
| | | H.36 | LIYDTSKLASGVPDRFSGSGSGTDYTLKISRVEAEDVGVYYCQQ |
| | | | WSSNPQYTFGQGTKLEIK |
| SEQ ID NO: 3221 | VL | D- | DIVLTQTPLSLSVTPGQPASISCSVSSSVSYMHWYLQKPGQPPKL |
| | | H.37 | LIYDTSKLASGVPDRFSGSGSGTDYTLKISRVEAEDVGVYYCQQ |
| | | | WSSNPQYTFGQGTKLEIK |
| SEQ ID NO: 3222 | VL | D- | DIQLIQSPSFLSASVGDRVSIICSVSSSVSYMHWYLQKPGKSPKLF |
| | | H.38 | IYDTSKLASGVSSRFSGRGSGTDYTLTIISLKPEDFAAYYCQQWS |
| | | | SNPQYTFGQGTKLEIK |
| SEQ ID NO: 3223 | VL | D- | DIVLTQTPLSSPVTLGQPASISCSVSSSVSYMHWYQQRPGQPPKL |
| | | H.39 | LIYDTSKLASGVPDRFSGSGAGTDYTLKISRVEAEDVGVYYCQQ |
| | | | WSSNPQYTFGQGTKLEIK |
| SEQ ID NO: 3224 | VL | D- | EITLTQSPAFMSATPGDKVNISCSVSSSVSYMHWYQQKPGEAPK |
| | | H.40 | FIIYDTSKLASGIPPRFSGSGYGTDYTLTINNIESEDAAYYYCQQW |
| | | | SSNPQYTFGQGTKLEIK |

Variable HEAVY chain (VH)

| | | | |
|---|---|---|---|
| SEQ ID NO: 3225 | VH | D-H.1 | EVQLVESGGGLVKPGGSLRLSCAVSGFTFRSYGMSWVRQAPGK |
| | | | GLEWVALISSGGSYTYYTDSVKGRFTISRDNSKNTLYLQMNSLK |
| | | | TEDTAVYYCSRHGGNFFDYWGQGTTVTVSS |
| SEQ ID NO: 3226 | VH | D-H.2 | EVQLVESGGALVKPGGSLRLSCAVSGFTFRSYGMSWVRQAPGK |
| | | | GLEWVALISSGGSYTYYTDSVKGRFTISRDNSKNTLYLQMNSLK |
| | | | TEDTAVYYCSRHGGNFFDYWGQGTTVTVSS |
| SEQ ID NO: 3227 | VH | D-H.3 | EVQLVESGGGLVQPGGSLRLSCAVSGFTFRSYGMSWVRQAPGK |
| | | | GLEWVALISSGGSYTYYTDSVKGRFTISRDNAKNTLYLQMNSLR |
| | | | AEDTAVYYCSRHGGNFFDYWGQGTTVTVSS |
| SEQ ID NO: 3228 | VH | D-H.4 | EVQLVESGGGLVQPGGSLRLSCAVSGFTFRSYGMSWVRQAPGK |
| | | | GLEWVALISSGGSYTYYTDSVKGRFTISRDNAKNSLYLQMNSLR |
| | | | AEDTAVYYCSRHGGNFFDYWGQGTTVTVSS |
| SEQ ID NO: 3229 | VH | D-H.5 | EVQLVESGGGLVQPGGSLRLSCAVSGFTFRSYGMSWVRQAPGK |
| | | | GLEWVALISSGGSYTYYTDSVKGRFTISRDNSKNSLYLQMNSLK |
| | | | TEDTAVYYCSRHGGNFFDYWGQGTTVTVSS |
| SEQ ID NO: 3230 | VH | D-H.6 | EVQLVESGGGLVQPGGSLRLSCAVSGFTFRSYGMSWVRQAPGK |
| | | | GLEWVALISSGGSYTYYTDSVKGRFTISRDNAKNSLYLQMNSLR |
| | | | AEDMAVYYCSRHGGNFFDYWGQGTTVTVSS |
| SEQ ID NO: 3231 | VH | D-H.7 | EVQLVESGGGLVQPGGSLRLSCAVSGFTFRSYGMSWVRQAPGK |
| | | | GLEWVALISSGGSYTYYTDSVKGQFTISRDNAKNTLYLQMNSLR |
| | | | AEDMAVYYCSRHGGNFFDYWGQGTTVTVSS |
| SEQ ID NO: 3232 | VH | D-H.8 | EVQLVESGGGLVKPGRSLRLSCTVSGFTFRSYGMSWVRQAPGK |
| | | | GLEWVALISSGGSYTYYTDSVKGRFTISRDNSKNILYLQMNSLK |
| | | | TEDTAVYYCSRHGGNFFDYWGQGTTVTVSS |
| SEQ ID NO: 3233 | VH | D-H.9 | EVQLVESGGGLVKPGGSLRLSCAVSGFTFRSYGMSWVRQAPGK |
| | | | GLEWVALISSGGSYTYYTDSVKGRFTISRDNAKNSLYLQMNSLR |
| | | | AEDTAVYYCSRHGGNFFDYWGQGTTVTVSS |
| SEQ ID NO: 3234 | VH | D- | EVQLVESGGGLVQPGGSLKLSCAVSGFTFRSYGMSWVRQASGK |
| | | H.10 | GLEWVALISSGGSYTYYTDSVKGRFTISRDNSKNTLYLQMNSLK |
| | | | TEDTAVYYCSRHGGNFFDYWGQGTTVTVSS |
| SEQ ID NO: 3235 | VH | D- | QVQLVESGGGVVQPGGSLRLSCAVSGFTFRSYGMSWVRQAPGK |
| | | H.11 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3236 | VH | D- | QVQLVESGGGVVQPGRSLRLSCAVSGFTFRSYGMSWVRQAPGK |
| | | H.12 | GLEWVALISSGGSYTYYTDSVKGRFTISRDNSKNTLYLQMSSLR |
| | | | AEDTAVYYCSRHGGNFFDYWGQGTTVTVSS |
| SEQ ID NO: 3237 | VH | D- | EVQLVESGGGLVQPGGSLRLSCPVSGFTFRSYGMSWVRQAPGK |
| | | H.13 | GLEWVALISSGGSYTYYTDSVKGRFTISRDNANNSLYLQMNSLR |
| | | | AEDTAVYYCSRHGGNFFDYWGQGTTVTVSS |
| SEQ ID NO: 3238 | VH | D- | EVQLVESGGGLVQPGRSLRLSCTVSGFTFRSYGMSWVRQAPGK |
| | | H.14 | GLEWVALISSGGSYTYYTDSVKGRFTISRDNSKNILYLQMNSLK |
| | | | TEDTAVYYCSRHGGNFFDYWGQGTTVTVSS |
| SEQ ID NO: 3239 | VH | D- | EVQLVESGGGLVQPGPSLRLSCTVSGFTFRSYGMSWVRQAPGK |
| | | H.15 | GLEWVALISSGGSYTYYTDSVKGRFTISRDNSKNILYLQMNSLK |
| | | | TEDTAVYYCSRHGGNFFDYWGQGTTVTVSS |
| SEQ ID NO: 3240 | VH | D- | EVQLVESGGGLVQPGGSLRLSCAVSGFTFRSYGMSWVRQAPGK |
| | | H.16 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3241 | VH | D- | EVQLVESGGGLVQPGGSLRLSCAVSGFTFRSYGMSWVRQAPGK |
| | | H.17 | GLEWVALISSGGSYTYYTDSVKGRFTISRDNAKNSLYLQMNSLR |
| | | | DEDTAVYYCSRHGGNFFDYWGQGTTVTVSS |
| SEQ ID NO: 3242 | VH | D- | QVQLVESGGGLVKPGGSLRLSCAVSGFTFRSYGMSWVRQAPGK |
| | | H.18 | GLEWVALISSGGSYTYYTDSVKGRFTISRDNAKNSLYLQMNSLR |
| | | | AEDTAVYYCSRHGGNFFDYWGQGTTVTVSS |
| SEQ ID NO: 3243 | VH | D- | QVQLVESGGGVVQPGRSLRLSCAVSGFTFRSYGMSWVRQAPGK |
| | | H.19 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3244 | VH | D- | EVQLLESGGGLVQPGGSLRLSCAVSGFTFRSYGMSWVRQAPGK |
| | | H.20 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3245 | VH | D- | EVQLVESGGGLVQPGGSLRLSCAVSGFTFRSYGMSWVRQAPGK |
| | | H.21 | GLEWVALISSGGSYTYYTDSVKGRFTISRHNSKNTLYLQMNSLR |
| | | | AEDTAVYYCSRHGGNFFDYWGQGTTVTVSS |
| SEQ ID NO: 3246 | VH | D- | EVQLVESGGGLIQPGGSLRLSCAVSGFTFRSYGMSWVRQPPGKG |
| | | H.22 | LEWVALISSGGSYTYYTDSVKGRFTISRDNSKNTLYLQMNSLRA |
| | | | EDTAVYYCSRHGGNFFDYWGQGTTVTVSS |
| SEQ ID NO: 3247 | VH | D- | EVQLVESGGGLIQPGGSLRLSCAVSGFTFRSYGMSWVRQAPGK |
| | | H.23 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3248 | VH | D- | EVQLVESGGGLVQPGRSLRLSCAVSGFTFRSYGMSWVRQAPGK |
| | | H.24 | GLEWVALISSGGSYTYYTDSVKGRFTISRDNAKNSLYLQMNSLR |
| | | | AEDTALYYCSRHGGNFFDYWGQGTTVTVSS |
| SEQ ID NO: 3249 | VH | D- | QVQLVESGGGVVQPGRSLRLSCAVSGFTFRSYGMSWVRQAPGK |
| | | H.25 | GLEWVALISSGGSYTYYTDSVKGRFTISRDNSKNRLYLQMNSLR |
| | | | AEDTAVYYCSRHGGNFFDYWGQGTTVTVSS |
| SEQ ID NO: 3250 | VH | D- | QVQLVESGGGVVQPGRSLRLSCAVSGFTFRSYGMSWVRQAPGK |
| | | H.26 | GLEWVALISSGGSYTYYTDSVKGRFTISRDNSKNTLYLQMNSLR |
| | | | AEGTAVYYCSRHGGNFFDYWGQGTTVTVSS |
| SEQ ID NO: 3251 | VH | D- | QVQLVESGGGVVQPGRSLRLSCAVSGFTFRSYGMSWVRQAPGK |
| | | H.27 | GLEWVALISSGGSYTYYTDSVKGRFAISRDNSKNTLYLQMNSLR |
| | | | AEDTAVYYCSRHGGNFFDYWGQGTTVTVSS |
| SEQ ID NO: 3252 | VH | D- | QVQLVDSGGGVVQPGRSLRLSCAVSGFTFRSYGMSWVRQAPG |
| | | H.28 | KGLEWVALISSGGSYTYYTDSVKGRFTISRDNSKNTLYLQMNSL |
| | | | RAEDTAVYYCSRHGGNFFDYWGQGTTVTVSS |
| SEQ ID NO: 3253 | VH | D- | EVQLVESGGGVVRPGGSLRLSCAVSGFTFRSYGMSWVRQAPGK |
| | | H.29 | GLEWVALISSGGSYTYYTDSVKGRFTISRDNAKNSLYLQMNSLR |
| | | | AEDTALYHCSRHGGNFFDYWGQGTTVTVSS |
| SEQ ID NO: 3254 | VH | D- | EVQLVESGGVVVQPGGSLRLSCAVSGFTFRSYGMSWVRQAPGK |
| | | H.30 | GLEWVALISSGGSYTYYTDSVKGRFTISRDNSKNSLYLQMNSLR |
| | | | AEDTALYYCSRHGGNFFDYWGQGTTVTVSS |
| SEQ ID NO: 3255 | VH | D- | EVQLVESGGGVVQPGGSLRLSCAVSGFTFRSYGMSWVRQAPGK |
| | | H.31 | GLEWVALISSGGSYTYYTDSVKGRFTISRDNSKNSLYLQMNSLR |
| | | | TEDTALYYCSRHGGNFFDYWGQGTTVTVSS |
| SEQ ID NO: 3256 | VH | D- | EVQLVESGGVVVQPGGSLRLSCAVSGFTFRSYGMSWVRQAPGK |
| | | H.32 | GLEWVALISSGGSYTYYTDSVKGRFTISRDNSKNSLYLQMNSLR |
| | | | TEDTALYYCSRHGGNFFDYWGQGTTVTVSS |
| SEQ ID NO: 3257 | VH | D- | EVQLVETGGGLIQPGGSLRLSCAVSGFTFRSYGMSWVRQAPGK |
| | | H.33 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3258 | VH | D- | EVQLVESGGGLVQPGGSLRLSCAVSGFTFRSYGMSWVRQATGK |
| | | H.34 | GLEWVALISSGGSYTYYTDSVKGRFTISRENAKNSLYLQMNSLR |
| | | | AGDTAVYYCSRHGGNFFDYWGQGTTVTVSS |
| SEQ ID NO: 3259 | VH | D- | EVQLVESRGVLVQPGGSLRLSCAVSGFTFRSYGMSWVRQAPGK |
| | | H.35 | GLEWVALISSGGSYTYYTDSVKGRFTISRDNSKNTLHLQMNSLR |
| | | | AEDTAVYYCSRHGGNFFDYWGQGTTVTVSS |
| SEQ ID NO: 3260 | VH | D- | EVQLVESGGGLVQPGRSLRLSCAVSGFTFRSYGMSWVRQAPGK |
| | | H.36 | GLEWVALISSGGSYTYYTDSVKGRFTISRDNAKNSLYLQMNSLR |
| | | | AEDMALYYCSRHGGNFFDYWGQGTTVTVSS |
| SEQ ID NO: 3261 | VH | D- | QVQLVESGGGLVQPGGSLRLSCSVSGFTFRSYGMSWVRQAPGK |
| | | H.37 | GLEWVALISSGGSYTYYTDSVKGRFTISRDNSKNTLYLQMNSLR |
| | | | AEDTAVYYCSRHGGNFFDYWGQGTTVTVSS |
| SEQ ID NO: 3262 | VH | D- | EVQLVESGGGLVQPGGSLRLSCSVSGFTFRSYGMSWVRQAPGK |
| | | H.38 | GLEWVALISSGGSYTYYTDSVKGRFTISRDNSKNTLYLQMSSLR |
| | | | AEDTAVYYCSRHGGNFFDYWGQGTTVTVSS |
| SEQ ID NO: 3263 | VH | D- | QVQLVESGGGVVQPGRSLRLSCAVSGFTFRSYGMSWVRQAPGK |
| | | H.39 | GLEWVALISSGGSYTYYTDSVKGRFTISRDNSTNTLFLQMNSLR |
| | | | AEDTAVYYCSRHGGNFFDYWGQGTTVTVSS |
| SEQ ID NO: 3264 | VH | D- | QVQLLESGGGLVKPGGSLRLSCAVSGFTFRSYGMSWVRQAPGK |
| | | H.40 | GLEWVALISSGGSYTYYTDSVKGRFTISRDNAKNSLYLQMNSLR |
| | | | AEDTAVYYCSRHGGNFFDYWGQGTTVTVSS |
| SEQ ID NO: 3265 | VH | D- | EVQLVESGEGLVQPGGSLRLSCAVSGFTFRSYGMSWVRQAPGK |
| | | H.41 | GLEWVALISSGGSYTYYTDSVKGRFTISRDNSKNTLYLQMGSLR |
| | | | AEDMAVYYCSRHGGNFFDYWGQGTTVTVSS |
| SEQ ID NO: 3266 | VH | D- | EVQLVESGGGLVQPGGSLRLSCAVSGFTFRSYGMSWVRQAPGK |
| | | H.42 | GLEWVALISSGGSYTYYTDSVKGRFTISRDNSKNTLYLQMGSLR |
| | | | AEDMAVYYCSRHGGNFFDYWGQGTTVTVSS |
| SEQ ID NO: 3267 | VH | D- | EVQLVESGGGLVQPGGSLRLSCSVSGFTFRSYGMSWVRQAPGK |
| | | H.43 | GLEWVALISSGGSYTYYTDSVKGRFTISRDNSKNTLYVQMSSLR |
| | | | AEDTAVYYCSRHGGNFFDYWGQGTTVTVSS |
| SEQ ID NO: 3268 | VH | D- | EVQLVESGGGLVQPGGSLRLSCAVSGFTFRSYGMSWVRQAPGK |
| | | H.44 | GLEWVALISSGGSYTYYTDSVKGRFIISRDNSRNSLYLQKNRRR |
| | | | AEDMAVYYCSRHGGNFFDYWGQGTTVTVSS |
| SEQ ID NO: 3269 | VH | D- | EVQLVESGGGLVQPGGSLRLSCAVSGFTFRSYGMSWVHQAPGK |
| | | H.45 | GLEWVALISSGGSYTYYTDSVKGRFIISRDNSRNTLYLQTNSLRA |
| | | | EDTAVYYCSRHGGNFFDYWGQGTTVTVSS |
| SEQ ID NO: 3270 | VH | D- | EVHLVESGGGLVQPGGALRLSCAVSGFTFRSYGMSWVRQATGK |
| | | H.46 | GLEWVALISSGGSYTYYTDSVKGRFTISRENAKNSLYLQMNSLR |
| | | | AGDTAVYYCSRHGGNFFDYWGQGTTVTVSS |
| SEQ ID NO: 3271 | VH | D- | EVQLVESGGGLVQPRGSLRLSCAVSGFTFRSYGMSWVRQAPGK |
| | | H.47 | GLEWVALISSGGSYTYYTDSVKGRFTISRDNSKNTLYLQMNNLR |
| | | | AEGTAVYYCSRHGGNFFDYWGQGTTVTVSS |
| SEQ ID NO: 3272 | VH | D- | EVQLVESGGGLVQPRGSLRLSCAVSGFTFRSYGMSWVRQAPGK |
| | | H.48 | GLEWVALISSGGSYTYYTDSVKGRFTISRDNSKNTLYLQMNNLR |
| | | | AEGTAAYYCSRHGGNFFDYWGQGTTVTVSS |
| SEQ ID NO: 3273 | VH | D- | QVQLVQSGAEVKKPGASVKVSCKVSGFTFRSYGMSWVRQAPG |
| | | H.49 | KGLEWVALISSGGSYTYYTDSVKGRFTITRDNSTNTLYMELSSL |
| | | | RSEDTAVYYCSRHGGNFFDYWGQGTTVTVSS |
| SEQ ID NO: 3274 | VH | D- | QVQLVQSGSELKKPGASVKVSCKVSGFTFRSYGMSWVRQAPGQ |
| | | H.50 | GLEWVALISSGGSYTYYTDSVKGRFVISRDNSVNTLYLQISSLKA |
| | | | EDTAVYYCSRHGGNFFDYWGQGTTVTVSS |

TABLE 45
Amino acid sequences for additional TCRβV antibodies. Amino acid and nucleotide
sequences for murine and humanized antibody molecules which bind to various TCRVB families
are disclosed. The amino acid the heavy and light chain CDRs, and the amino acid and
nucleotide sequences of the heavy and light chain variable regions, and the heavy and
light chains are shown. Antibodies disclosed in the table include, IMMU222 and JOVI-3.
IMMU 222 binds human TCRβV 6-5, TCRβV 6-6, or TCRβV 6-9 (TCRβV13.1 per old nomenclature).
JOVI-3 binds human TCRβV 28 (TCRβV3.1 per old nomenclature).
IMMU222 (murine) also referred to as BJ1461; or Antibody I
Binds to human TCRVβ 6-5, 6-6, 6-9

| | | |
|---|---|---|
| SEQ ID NO: 1141 | HC CDR1 (Kabat) | SYAMS |
| SEQ ID NO: 1142 | HC CDR2 (Kabat) | HISNGGDYIYYADTVKG |
| SEQ ID NO: 1143 | HC CDR3 (Kabat) | PSYYSDPWFFDV |
| SEQ ID NO: 1144 | HC CDR1 (Chothia) | GFTFRSY |
| SEQ ID NO: 1145 | HC CDR2 (Chothia) | SNGGDY |
| SEQ ID NO: 1143 | HC CDR3 (Chothia) | PSYYSDPWFFDV |
| SEQ ID NO: 1146 | HC CDR1 (Combined) | GFTFRSYAMS |
| SEQ ID NO: 1142 | HC CDR2 (Combined) | HISNGGDYIYYADTVKG |
| SEQ ID NO: 1143 | HC CDR3(Combined) | PSYYSDPWFFDV |
| SEQ ID NO: 1147 | LC CDR1 (Kabat) | SAGSSVSFMH |
| SEQ ID NO: 1148 | LC CDR2 (Kabat) | DTSKLAS |
| SEQ ID NO: 1149 | LC CDR3 (Kabat) | LQGSGFPLT |
| SEQ ID NO: 1150 | LC CDR1 (Chothia) | GSSVSF |
| SEQ ID NO: 1148 | LC CDR2 (Chothia) | DTSKLAS |
| SEQ ID NO: 1149 | LC CDR3 (Chothia) | LQGSGFPLT |
| SEQ ID NO: 1147 | LC CDR1 (Combined) | SAGSSVSFMH |
| SEQ ID NO: 1148 | LC CDR2 (Combined) | DTSKLAS |
| SEQ ID NO: 1149 | LC CDR3(Combined) | LQGSGFPLT |
| SEQ ID NO: 1151 | VL | ENVLTQSPAIMSASPGEKVTMTCSAGSSVSFMHWYQQKSSTSP |
| | | KLWIYDTSKLASGVPGRFSGSGSGNSFSLTISSMEAEDVAIYYC |
| | | LQGSGFPLTFGSGTKLEIK |
| SEQ ID NO: 1152 | VH | DVKLVESGEGLVKPGGSLKLSCAASGFTFRSYAMSWVRQTPE |
| | | KRLEWVAHISNGGDYIYYADTVKGRFTISRDNARNTLYLQMS |
| | | SLKSEDTAMYYCTRPSYYSDPWFFDVWGTGTTVTVSS |

VH for IMMU222 (humanized) also referred to as Antibody I-H
Binds to human TCRVβ 6-5, 6-6, 6-9

| | | |
|---|---|---|
| SEQ ID NO: 1153 | VH-1 | EVQLVESGGGLVQPGGSLRLSCAASGFTFRSYAMSWVRQAPG |
| | | KGLEWVAHISNGGDYIYYADTVKGRFTISRDNAKNSLYLQMN |
| | | SLRAEDTAVYYCTRPSYYSDPWFFDVWGQGTTVTVSS |
| SEQ ID NO: 1154 | VH-2 | QVQLVESGGGVVQPGRSLRLSCAASGFTFRSYAMSWVRQAPG |
| | | KGLEWVAHISNGGDYIYYADTVKGRFTISRDNSKNTLYLQMS |
| | | SLRAEDTAVYYCTRPSYYSDPWFFDVWGQGTTVTVSS |
| SEQ ID NO: 1155 | VH-3 | EVQLVESGGGLVQPGGSLRLSCAASGFTFRSYAMSWVRQAPG |
| | | KGLEWVAHISNGGDYIYYADTVKGRFTISRDNSKNTLYLQMN |
| | | SLRAEDTAVYYCTRPSYYSDPWFFDVWGQGTTVTVSS |
| SEQ ID NO: 1156 | VH-4 | QVQLVQSGSELKKPGASVKVSCKASGFTFRSYAMSWVRQAPG |
| | | QGLEWVAHISNGGDYIYYADTVKGRFVISRDNSVNTLYLQISS |
| | | LKAEDTAVYYCTRPSYYSDPWFFDVWGQGTTVTVSS |
| SEQ ID NO: 1157 | VH-5 | QVQLVQSGAEVKKPGASVKVSCKASGFTFRSYAMSWVRQAP |
| | | GQRLEWVAHISNGGDYIYYADTVKGRFTITRDNSANTLYMEL |
| | | SSLRSEDTAVYYCTRPSYYSDPWFFDVWGQGTTVTVSS |

VL for IMMU222 (humanized)) also referred to as Antibody I-H
Binds to human TCRVβ 6-5, 6-6, 6-9

| | | |
|---|---|---|
| SEQ ID NO: 1158 | VL-1 | ENVLTQSPATLSLSPGERATLSCSAGSSVSFMHWYQQKPGQAP |
| | | KLLIYDTSKLASGIPARFSGSGSGNDFTLTISSLEPEDFAVYYCL |
| | | QGSGFPLTFGQGTKLEIK |
| SEQ ID NO: 1159 | VL-2 | ENVLTQSPDFQSVTPKEKVTITCSAGSSVSFMHWYQQKPDQSP |
| | | KLLIYDTSKLASGVPSRFSGSGSGNDFTLTINSLEAEDAATYYC |
| | | LQGSGFPLTFGQGTKLEIK |
| SEQ ID NO: 1160 | VL-3 | DNQLTQSPSSLSASVGDRVTITCSAGSSVSFMHWYQQKPGKVP |
| | | KLLIYDTSKLASGVPSRFSGSGSGNDFTLTISSLQPEDVATYYC |
| | | LQGSGFPLTFGQGTKLEIK |
| SEQ ID NO: 1161 | VL-4 | ANQLTQSPSSLSASVGDRVTITCSAGSSVSFMHWYQQKPGKAP |
| | | KLLIYDTSKLASGVPSRFSGSGSGNDFTLTISSLQPEDFATYYCL |
| | | QGSGFPLTFGQGTKLEIK |
| SEQ ID NO: 1162 | VL-5 | DNVLTQSPDSLAVSLGERATINCSAGSSVSFMHWYQQKPGQPP |
| | | KLLIYDTSKLASGVPDRFSGSGSGNDFTLTISSLQAEDVAVYYC |
| | | LQGSGFPLTFGQGTKLEIK |

JOVI-3 (murine), also referred to as BJ1187 or Antibody K
Binds to human TCRVβ 28

| | | |
|---|---|---|
| SEQ ID NO: 1185 | HC CDR1 (Kabat) | GSWMN |
| SEQ ID NO: 1186 | HC CDR2 (Kabat) | RIYPGDGDTDYSGKFKG |
| SEQ ID NO: 1187 | HC CDR3 (Kabat) | SGYFNYVPVFDY |
| SEQ ID NO: 1188 | HC CDR1 (Chothia) | GYTFSGS |
| SEQ ID NO: 1189 | HC CDR2 (Chothia) | YPGDGD |
| SEQ ID NO: 1187 | HC CDR3 (Chothia) | SGYFNYVPVFDY |
| SEQ ID NO: 1190 | HC CDR1 (Combined) | GYTFSGSWMN |
| SEQ ID NO: 1186 | HC CDR2 (Combined) | RIYPGDGDTDYSGKFKG |
| SEQ ID NO: 1187 | HC CDR3(Combined) | SGYFNYVPVFDY |
| SEQ ID NO: 1191 | LC CDR1 (Kabat) | SANSTVGYIH |
| SEQ ID NO: 1192 | LC CDR2 (Kabat) | TTSNLAS |
| SEQ ID NO: 1193 | LC CDR3 (Kabat) | HQWSFYPT |
| SEQ ID NO: 1194 | LC CDR1 (Chothia) | NSTVGY |
| SEQ ID NO: 1192 | LC CDR2 (Chothia) | TTSNLAS |
| SEQ ID NO: 1193 | LC CDR3 (Chothia) | HQWSFYPT |
| SEQ ID NO: 1191 | LC CDR1 (Combined) | SANSTVGYIH |
| SEQ ID NO: 1192 | LC CDR2 (Combined) | TTSNLAS |
| SEQ ID NO: 1193 | LC CDR3(Combined) | HQWSFYPT |
| SEQ ID NO: 1195 | VL | QIVLTQSPAIMSASLGEEIALTCSANSTVGYIHWYQQKSGTSPK |
| | | LLIYTTSNLASGVPSRFSGSGSGTFYSLTISSVEAEDAADYFCH |
| | | QWSFYPTFGGGTKLEIK |
| SEQ ID NO: 1196 | VH | QIQLQQSGPEVVKPGASVQISCKASGYTFSGSWMNWVKQRPG |
| | | KGLEWIGRIYPGDGDTDYSGKFKGRATLTADKSSSTAYMRLSS |
| | | LTSEDSAVYFCARSGYFNYVPVFDYWGQGTTLSVSS |

VH for JOVI-3 (humanized) also referred to as Antibody K-H
Binds to human TCRVβ 28

| | | |
|---|---|---|
| SEQ ID NO: 1197 | VH-1 | QIQLVQSGAEVKKPGASVKVSCKASGYTFSGSWMNWVRQAP |
| | | GQGLEWIGRIYPGDGDTDYSGKFKGRATLTADKSTSTAYMEL |
| | | SSLRSEDTAVYYCARSGYFNYVPVFDYWGQGTTVTVSS |
| SEQ ID NO: 1198 | VH-2 | QIQLVQSGAEVKKPGSSVKVSCKASGYTFSGSWMNWVRQAP |
| | | GQGLEWIGRIYPGDGDTDYSGKFKGRATLTADKSTSTAYMEL |
| | | SSLRSEDTAVYYCARSGYFNYVPVFDYWGQGTTVTVSS |
| SEQ ID NO: 1199 | VH-3 | EIQLVQSGAEVKKPGESLKISCKASGYTFSGSWMNWVRQMPG |
| | | KGLEWIGRIYPGDGDTDYSGKFKGQATLSADKSISTAYLQWSS |
| | | LKASDTAMYYCARSGYFNYVPVFDYWGQGTTVTVSS |
| SEQ ID NO: 1200 | VH-4 | QIQLVQSGSELKKPGASVKVSCKASGYTFSGSWMNWVRQAP |
| | | GQGLEWIGRIYPGDGDTDYSGKFKGRAVLSADKSVSTAYLQIS |
| | | SLKAEDTAVYYCARSGYFNYVPVFDYWGQGTTVTVSS |
| SEQ ID NO: 1201 | VH-5 | QIQLVQSGSELKKPGASVKVSCKASGYTFSGSWMNWVRQAP |
| | | GQGLEWIGRIYPGDGDTDYSGKFKGRAVLSADKSVSMAYLQI |
| | | SSLKAEDTAVYYCARSGYFNYVPVFDYWGQGTTVTVSS |
| SEQ ID NO: 1202 | VH-6 | EIQLVESGGGLVQPGRSLRLSCTASGYTFSGSWMNWVRQAPG |
| | | KGLEWIGRIYPGDGDTDYSGKFKGRATLSADKSKSIAYLQMN |
| | | SLKTEDTAVYYCARSGYFNYVPVFDYWGQGTTVTVSS |

VL for JOVI-3 (humanized) also referred to as Antibody K-H
Binds to human TCRVβ 28

| | | |
|---|---|---|
| SEQ ID NO: 1203 | VL-1 | EIVLTQSPATLSLSPGERATLSCSANSTVGYIHWYQQKPGQAP |
| | | KLLIYTTSNLASGIPARFSGSGSGTDYTLTISSLEPEDFAVYFCH |
| | | QWSFYPTFGQGTKLEIK |
| SEQ ID NO: 1204 | VL-2 | DIQLTQSPSFLSASVGDRVTITCSANSTVGYIHWYQQKPGKAP |
| | | KLLIYTTSNLASGVPSRFSGSGSGTEYTLTISSLQPEDFATYFCH |
| | | QWSFYPTFGQGTKLEIK |
| SEQ ID NO: 1205 | VL-3 | EIVLTQSPATLSLSPGERATLSCSANSTVGYIHWYQQKPGQAP |
| | | KLLIYTTSNLASGIPARFSGSGPGTDYTLTISSLEPEDFAVYFCH |
| | | QWSFYPTFGQGTKLEIK |
| SEQ ID NO: 1206 | VL-4 | DIVLTQSPDSLAVSLGERATINCSANSTVGYIHWYQQKPGQPP |
| | | KLLIYTTSNLASGVPDRFSGSGSGTDYTLTISSLQAEDVAVYFC |
| | | HQWSFYPTFGQGTKLEIK |
| SEQ ID NO: 1207 | VL-5 | EIVLTQSPDFQSVTPKEKVTITCSANSTVGYIHWYQQKPDQSPK |
| | | LLIYTTSNLASGVPSRFSGSGSGTDYTLTINSLEAEDAATYFCH |
| | | QWSFYPTFGQGTKLEIK |

TABLE 46
Exemplary molecules or agents that comprise a first binder that binds to a first target
molecule, wherein the exemplary first binder comprises anti-TCRβ V20 antibodies. Exemplary
first target molecule comprises TCRVB 20 (e.g., TCRVB 20-1). Exemplary anti-TCR V20
antibodies include, but are not limited to, BLM0274 and BLM0276, which binds to human TCRβV
20-1. CDRs are underlined.
BLM0274, also referred to as BJ1325 or BJ1329
Binds to human TCRVβ 20-1

| | | |
|---|---|---|
| SEQ ID NO: 3318 | Heavy chain (VH- | QVQLVQSGAEVKKPGASVKVSCKASGFNIKSAYMHWVRQA |
| | CH) | PGQEPGCMGRIDPATGKTKYAPKFQARVTMTADTSINTAYTE |
| | | LSSLRSEDTATYYCARSLNWDYGLDYWGQGTLVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKRVEPKSCDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVCTLPPSREEMTKNQVSLSCAVKGFYP |
| | | SDIAVEWESNGQPENNYKTTPPVLDSDGSFFLVSKLTVDKSR |
| | | WQQGNVFSCSVMHEALHNHYTQKSLSLSPGK |
| SEQ ID NO: 4710 | Heavy chain (VH- | QVQLVQSGAEVKKPGASVKVSCKASGFNIKSAYMHWVRQA |
| | CH) | PGQEPGCMGRIDPATGKTKYAPKFQARVTMTADTSINTAYTE |
| | | LSSLRSEDTATYYCARSLNWDYGLDYWGQGTLVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKRVEPKSCDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVCTLPPSREEMTKNQVSLSCAVKGFYP |
| | | SDIAVEWESNGQPENNYKTTPPVLDSDGSFFLVSKLTVDKSR |
| | | WQQGNVFSCSVMHEALHNHYTQKSLSLSP |
| SEQ ID NO: 3319 | Light chain (VL-CL) | EIVLTQSPATLSLSPGERATLSCRASKSVSILGTHLIHWYQQKP |
| | | GLAPRLLIYAASNLESGIPDRFSGSGSETDFTLTISRLEPEDFAV |
| | | YFCQQSIEDPWTFGGGTKVEIKRTVAAPSVFIFPPSDEQLKSG |
| | | TASVVCLLNNFYPREAKVQWKVDNALQSGNSQESVTEQDSK |
| | | DSTYSLSSTLTLSKADYEKHKVYACEVTHQGLSSPVTKSFNR |
| | | GEC |
| SEQ ID NO: 3340 | IL-2 C125A-Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGSGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVYTLPPCREEMTKNQVSLWCLVKGFY |
| | | PSDIAVEWESNGQPENNYKTTPPVLDSDGSFFLYSKLTVDKS |
| | | RWQQGNVFSCSVMHEALHNHYTQKSLSLSPGK |
| SEQ ID NO: 4706 | IL-2 C125A-Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGSGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVYTLPPCREEMTKNQVSLWCLVKGFY |
| | | PSDIAVEWESNGQPENNYKTTPPVLDSDGSFFLYSKLTVDKS |
| | | RWQQGNVFSCSVMHEALHNHYTQKSLSLSP |
| SEQ ID NO: 3321 | HC CDR1 | GFNIKSAYMH |
| SEQ ID NO: 3322 | HC CDR2 | RIDPATGKTKYAPKFQA |
| SEQ ID NO: 3323 | HC CDR3 | SLNWDYGLDY |
| SEQ ID NO: 3324 | LC CDR1 | RASKSVSILGTHLIH |
| SEQ ID NO: 3325 | LC CDR2 | AASNLES |
| SEQ ID NO: 3326 | LC CDR3 | QQSIEDPWT |
| SEQ ID NO: 3448 | VH | QVQLVQSGAEVKKPGASVKVSCKASGFNIKSAYMHWVRQAP |
| | | GQEPGCMGRIDPATGKTKYAPKFQARVTMTADTSINTAYTEL |
| | | SSLRSEDTATYYCARSLNWDYGLDYWGQGTLVTVSS |
| SEQ ID NO: 3449 | VL | EIVLTQSPATLSLSPGERATLSCRASKSVSILGTHLIHWYQQKP |
| | | GLAPRLLIYAASNLESGIPDRFSGSGSETDFTLTISRLEPEDFAV |
| | | YFCQQSIEDPWTFGGGTKVEIK |
| SEQ ID NO: 3649 | Heavy chain constant | ASTKGPSVFPLAPSSKSTSGGTAALGCLVKDYFPEPVTVSWNS |
| | region (CH1-CH2- | GALTSGVHTFPAVLQSSGLYSLSSVVTVPSSSLGTQTYICNVNH |
| | CH3) Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSPGK |
| SEQ ID NO: 8687 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSP |
| SEQ ID NO: 3447 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSPGK |
| SEQ ID NO: 511 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSP |
| SEQ ID NO: 3644 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 2270 | IL-2 C125A | APTSSSTKKTQLQLEHLLLDLQMILNGINNYKNPKLTRMLTFK |
| | | FYMPKKATELKHLQCLEEELKPLEEVLNLAQSKNFHLRPRDLI |
| | | SNINVIVLELKGSETTFMCEYADETATIVEFLNRWITFAQSIIST |
| | | LT |
| SEQ ID NO: 3308 | Linker | GGGGSGGGGS |
| SEQ ID NO: 3648 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |
| SEQ ID NO: 508 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSP |

BLM0276, also referred to as BJ1324, BJ1330
Binds to human TCRVβ 20-1

| | | |
|---|---|---|
| SEQ ID NO: 3328 | Heavy chain (VH- CH) | QVQLVQSGAEVKKPGASVKVSCKASGFNIKSAYMHWVRQA |
| | | PGQGLEWMGRIDPATGKTKYAPKFQARVTMTADTSTNTAY |
| | | MELSSLRSEDTAVYYCARSLNWDYGLDYWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVNH |
| | | KPSNTKVDKRVEPKSCDKTHTCPPCPAPELLGGPSVFLFPPKP |
| | | KDTLMISRTPEVTCVVVDVSHEDPEVKFNWYVDGVEVHNAK |
| | | TKPREEQYASTYRVVSVLTVLHQDWLNGKEYKCKVSNKALP |
| | | APIEKTISKAKGQPREPQVCTLPPSREEMTKNQVSLSCAVKGF |
| | | YPSDIAVEWESNGQPENNYKTTPPVLDSDGSFFLVSKLTVDK |
| | | SRWQQGNVFSCSVMHEALHNHYTQKSLSLSPGK |
| SEQ ID NO: 4711 | Heavy chain (VH- CH) | QVQLVQSGAEVKKPGASVKVSCKASGFNIKSAYMHWVRQA |
| | | PGQGLEWMGRIDPATGKTKYAPKFQARVTMTADTSTNTAY |
| | | MELSSLRSEDTAVYYCARSLNWDYGLDYWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVNH |
| | | KPSNTKVDKRVEPKSCDKTHTCPPCPAPELLGGPSVFLFPPKP |
| | | KDTLMISRTPEVTCVVVDVSHEDPEVKFNWYVDGVEVHNAK |
| | | TKPREEQYASTYRVVSVLTVLHQDWLNGKEYKCKVSNKALP |
| | | APIEKTISKAKGQPREPQVCTLPPSREEMTKNQVSLSCAVKGF |
| | | YPSDIAVEWESNGQPENNYKTTPPVLDSDGSFFLVSKLTVDK |
| | | SRWQQGNVFSCSVMHEALHNHYTQKSLSLSP |
| SEQ ID NO: 3329 | Light chain (VL-CL) | EIVLTQSPGTLSLSPGERATLSCRASKSVSILGTHLIHWYQQKP |
| | | GQAPRLLIYAASNLESGIPDRFSGSGSETDFTLTISRLEPEDFA |
| | | VYFCQQSIEDPWTFGGGTKVEIKRTVAAPSVFIFPPSDEQLKS |
| | | GTASVVCLLNNFYPREAKVQWKVDNALQSGNSQESVTEQDS |
| | | KDSTYSLSSTLTLSKADYEKHKVYACEVTHQGLSSPVTKSFN |
| | | RGEC |
| SEQ ID NO: 3340 | IL-2 C125A-Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGSGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVYTLPPCREEMTKNQVSLWCL VKGFY |
| | | PSDIAVEWESNGQPENNYKTTPPVLDSDGSFFLYSKLTVDKS |
| | | RWQQGNVFSCSVMHEALHNHYTQKSLSLSPGK |
| SEQ ID NO: 4706 | IL-2 C125A-Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGSGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVYTLPPCREEMTKNQVSLWCLVKGFY |
| | | PSDIAVEWESNGQPENNYKTTPPVLDSDGSFFLYSKLTVDKS |
| | | RWQQGNVFSCSVMHEALHNHYTQKSLSLSP |
| SEQ ID NO: 3321 | HC CDR1 | GFNIKSAYMH |
| SEQ ID NO: 3322 | HC CDR2 | RIDPATGKTKYAPKFQA |
| SEQ ID NO: 3323 | HC CDR3 | SLNWDYGLDY |
| SEQ ID NO: 3324 | LC CDR1 | RASKSVSILGTHLIH |
| SEQ ID NO: 3325 | LC CDR2 | AASNLES |
| SEQ ID NO: 3326 | LC CDR3 | QQSIEDPWT |
| SEQ ID NO: 3450 | VH | QVQLVQSGAEVKKPGASVKVSCKASGFNIKSAYMHWVRQAP |
| | | GQGLEWMGRIDPATGKTKYAPKFQARVTMTADTSTNTAYME |
| | | LSSLRSEDTAVYYCARSLNWDYGLDYWGQGTLVTVSS |
| SEQ ID NO: 8688 | VL | EIVLTQSPGTLSLSPGERATLSCRASKSVSILGTHLIHWYQQKP |
| | | GQAPRLLIYAASNLESGIPDRFSGSGSETDFTLTISRLEPEDFAV |
| | | YFCQQSIEDPWTFGGGTKVEIK |
| SEQ ID NO: 3649 | Heavy chain constant | ASTKGPSVFPLAPSSKSTSGGTAALGCLVKDYFPEPVTVSWNS |
| | region (CH1-CH2- | GALTSGVHTFPAVLQSSGLYSLSSVVTVPSSSLGTQTYICNVNH |
| | CH3) Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSPGK |
| SEQ ID NO: 3451 | Heavy chain constant | ASTKGPSVFPLAPSSKSTSGGTAALGCLVKDYFPEPVTVSWNS |
| | region (CH1-CH2- | GALTSGVHTFPAVLQSSGLYSLSSVVTVPSSSLGTQTYICNVNH |
| | CH3) Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSP |
| SEQ ID NO: 3447 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSPGK |
| SEQ ID NO: 511 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSP |
| SEQ ID NO: 3644 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 2270 | IL-2 C125A | APTSSSTKKTQLQLEHLLLDLQMILNGINNYKNPKLTRMLTFK |
| | | FYMPKKATELKHLQCLEEELKPLEEVLNLAQSKNFHLRPRDLI |
| | | SNINVIVLELKGSETTFMCEYADETATIVEFLNRWITFAQSIIST |
| | | LT |
| SEQ ID NO: 3308 | Linker | GGGGSGGGGS |
| SEQ ID NO: 3648 | Heavy chain hinge-Fc | DKTHTCPPCPAPELLGGPSVFLFPPKPKDTLMISRTPEVTCVVV |
| | region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |
| SEQ ID NO: 508 | Heavy chain hinge-Fc | DKTHTCPPCPAPELLGGPSVFLFPPKPKDTLMISRTPEVTCVVV |
| | region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSP |

BLM0000

| | | |
|---|---|---|
| SEQ ID NO: 3328 | Heavy chain (VH-CH) | QVQLVQSGAEVKKPGASVKVSCKASGFNIKSAYMHWVRQA |
| | | PGQGLEWMGRIDPATGKTKYAPKFQARVTMTADTSTNTAY |
| | | MELSSLRSEDTAVYYCARSLNWDYGLDYWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVNH |
| | | KPSNTKVDKRVEPKSCDKTHTCPPCPAPELLGGPSVFLFPPKP |
| | | KDTLMISRTPEVTCVVVDVSHEDPEVKFNWYVDGVEVHNAK |
| | | TKPREEQYASTYRVVSVLTVLHQDWLNGKEYKCKVSNKALP |
| | | APIEKTISKAKGQPREPQVCTLPPSREEMTKNQVSLSCAVKGF |
| | | YPSDIAVEWESNGQPENNYKTTPPVLDSDGSFFLVSKLTVDK |
| | | SRWQQGNVFSCSVMHEALHNHYTQKSLSLSPGK |
| SEQ ID NO: 4711 | Heavy chain (VH-CH) | QVQLVQSGAEVKKPGASVKVSCKASGFNIKSAYMHWVRQA |
| | | PGQGLEWMGRIDPATGKTKYAPKFQARVTMTADTSTNTAY |
| | | MELSSLRSEDTAVYYCARSLNWDYGLDYWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVNH |
| | | KPSNTKVDKRVEPKSCDKTHTCPPCPAPELLGGPSVFLFPPKP |
| | | KDTLMISRTPEVTCVVVDVSHEDPEVKFNWYVDGVEVHNAK |
| | | TKPREEQYASTYRVVSVLTVLHQDWLNGKEYKCKVSNKALP |
| | | APIEKTISKAKGQPREPQVCTLPPSREEMTKNQVSLSCAVKGF |
| | | YPSDIAVEWESNGQPENNYKTTPPVLDSDGSFFLVSKLTVDK |
| | | SRWQQGNVFSCSVMHEALHNHYTQKSLSLSP |
| SEQ ID NO: 3329 | Light chain (VL-CL) | EIVLTQSPGTLSLSPGERATLSCRASKSVSILGTHLIHWYQQKP |
| | | GQAPRLLIYAASNLESGIPDRFSGSGSETDFTLTISRLEPEDFA |
| | | VYFCQQSIEDPWTFGGGTKVEIKRTVAAPSVFIFPPSDEQLKS |
| | | GTASVVCLLNNFYPREAKVQWKVDNALQSGNSQESVTEQDS |
| | | KDSTYSLSSTLTLSKADYEKHKVYACEVTHQGLSSPVTKSFN |
| | | RGEC |
| SEQ ID NO: 8703 | IL-2 C125A-Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGSGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVYTLPPCREEMTKNQVSLWCLVKGFY |
| | | PSDIAVEWESNGQPENNYKTTPPVLDSDGSFFLYSKLTVDKS |
| | | RWQQGNVFSCSVMHEALHNHYTQKSLSLSPGK |
| SEQ ID NO: 3350 | IL-2 C125A-Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGSGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVYTLPPCREEMTKNQVSLWCLVKGFY |
| | | PSDIAVEWESNGQPENNYKTTPPVLDSDGSFFLYSKLTVDKS |
| | | RWQQGNVFSCSVMHEALHNHYTQKSLSLSP |
| SEQ ID NO: 3321 | HC CDR1 | GFNIKSAYMH |
| SEQ ID NO: 3322 | HC CDR2 | RIDPATGKTKYAPKFQA |
| SEQ ID NO: 3323 | HC CDR3 | SLNWDYGLDY |
| SEQ ID NO: 3324 | LC CDR1 | RASKSVSILGTHLIH |
| SEQ ID NO: 3325 | LC CDR2 | AASNLES |
| SEQ ID NO: 3326 | LC CDR3 | QQSIEDPWT |
| SEQ ID NO: 3450 | VH | QVQLVQSGAEVKKPGASVKVSCKASGFNIKSAYMHWVRQAP |
| | | GQGLEWMGRIDPATGKTKYAPKFQARVTMTADTSTNTAYME |
| | | LSSLRSEDTAVYYCARSLNWDYGLDYWGQGTLVTVSS |
| SEQ ID NO: 8688 | VL | EIVLTQSPGTLSLSPGERATLSCRASKSVSILGTHLIHWYQQKP |
| | | GQAPRLLIYAASNLESGIPDRFSGSGSETDFTLTISRLEPEDFAV |
| | | YFCQQSIEDPWTFGGGTKVEIK |
| SEQ ID NO: 3649 | Heavy chain constant | ASTKGPSVFPLAPSSKSTSGGTAALGCLVKDYFPEPVTVSWNS |
| | region (CH1-CH2- | GALTSGVHTFPAVLQSSGLYSLSSVVTVPSSSLGTQTYICNVNH |
| | CH3) Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSPGK |
| SEQ ID NO: 3451 | Heavy chain constant | ASTKGPSVFPLAPSSKSTSGGTAALGCLVKDYFPEPVTVSWNS |
| | region (CH1-CH2- | GALTSGVHTFPAVLQSSGLYSLSSVVTVPSSSLGTQTYICNVNH |
| | CH3) Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSP |
| SEQ ID NO: 3447 | Heavy chain hinge-Fc | DKTHTCPPCPAPELLGGPSVFLFPPKPKDTLMISRTPEVTCVVV |
| | region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSPGK |
| SEQ ID NO: 511 | Heavy chain hinge-Fc | DKTHTCPPCPAPELLGGPSVFLFPPKPKDTLMISRTPEVTCVVV |
| | region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSP |
| SEQ ID NO: 3644 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 2270 | IL-2 C125A | APTSSSTKKTQLQLEHLLLDLQMILNGINNYKNPKLTRMLTFK |
| | | FYMPKKATELKHLQCLEEELKPLEEVLNLAQSKNFHLRPRDLI |
| | | SNINVIVLELKGSETTFMCEYADETATIVEFLNRWITFAQSIIST |
| | | LT |
| SEQ ID NO: 3308 | Linker | GGGGSGGGGS |
| SEQ ID NO: 3648 | Heavy chain hinge-Fc | DKTHTCPPCPAPELLGGPSVFLFPPKPKDTLMISRTPEVTCVVV |
| | region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |
| SEQ ID NO: 3454 | Heavy chain hinge-Fc | DKTHTCPPCPAPELLGGPSVFLFPPKPKDTLMISRTPEVTCVVV |
| | region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSP |

TABLE 47
Exemplary molecule comprising an antigen binding domain that binds to a T cell
receptor beta variable region (TCRβV), wherein the exemplary antigen binding domain
comprises anti-TCRβ V28 antibodies. Exemplary antigen binding domain comprises TCRβ V28.
Exemplary anti-TCRβ V28 antibodies include, but are not limited to, BLM0286, which binds to
human TCRβV 28. CDRs are underlined.
BLM0286, also referred to as BL203 or BJ1319
Binds to human TCRVβ 28

| | | |
|---|---|---|
| SEQ ID NO: 3358 | Heavy chain (VH- | QIQLQQSGPEVVKPGASVQISCKASGYTFSGSWMNWVKQRP |
| | CH) | GKGLEWIGRIYPGDGDTDYSGKFKGRATLTADKSSSTAYMR |
| | | LSSLTSEDSAVYFCARSGYFNYVPVFDYWGQGTTLSVSSAST |
| | | KGPSVFPLAPSSKSTSGGTAALGCLVKDYFPEPVTVSWNSGA |
| | | LTSGVHTFPAVLQSSGLYSLSSVVTVPSSSLGTQTYICNVNHK |
| | | PSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFY |
| | | PSDIAVEWESNGQPENNYKTTPPVLDSDGSFFLVSKLTVDKS |
| | | RWQQGNVFSCSVMHEALHNHYTQKSLSLSPGK |
| SEQ ID NO: 4712 | Heavy chain (VH- | QIQLQQSGPEVVKPGASVQISCKASGYTFSGSWMNWVKQRP |
| | CH) | GKGLEWIGRIYPGDGDTDYSGKFKGRATLTADKSSSTAYMR |
| | | LSSLTSEDSAVYFCARSGYFNYVPVFDYWGQGTTLSVSSAST |
| | | KGPSVFPLAPSSKSTSGGTAALGCLVKDYFPEPVTVSWNSGA |
| | | LTSGVHTFPAVLQSSGLYSLSSVVTVPSSSLGTQTYICNVNHK |
| | | PSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFY |
| | | PSDIAVEWESNGQPENNYKTTPPVLDSDGSFFLVSKLTVDKS |
| | | RWQQGNVFSCSVMHEALHNHYTQKSLSLSP |
| SEQ ID NO: 3359 | Light chain (VL-CL) | QIVLTQSPAIMSASLGEEIALTCSANSTVGYIHWYQQKSGTSP |
| | | KLLIYTTSNLASGVPSRFSGSGSGTFYSLTISSVEAEDAADYFC |
| | | HQWSFYPTFGGGTKLEIKRTVAAPSVFIFPPSDEQLKSGTASV |
| | | VCLLNNFYPREAKVQWKVDNALQSGNSQESVTEQDSKDSTY |
| | | SLSSTLTLSKADYEKHKVYACEVTHQGLSSPVTKSFNRGEC |
| SEQ ID NO: 3340 | IL-2 C125A-Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGSGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVYTLPPCREEMTKNQVSLWCLVKGFY |
| | | PSDIAVEWESNGQPENNYKTTPPVLDSDGSFFLYSKLTVDKS |
| | | RWQQGNVFSCSVMHEALHNHYTQKSLSLSPGK |
| SEQ ID NO: 4706 | IL-2 C125A-Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGSGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVYTLPPCREEMTKNQVSLWCLVKGFY |
| | | PSDIAVEWESNGQPENNYKTTPPVLDSDGSFFLYSKLTVDKS |
| | | RWQQGNVFSCSVMHEALHNHYTQKSLSLSP |
| SEQ ID NO: 1190 | HC CDR1 | GYTFSGSWMN |
| SEQ ID NO: 1186 | HC CDR2 | RIYPGDGDTDYSGKFKG |
| SEQ ID NO: 1187 | HC CDR3 | SGYFNYVPVFDY |
| SEQ ID NO: 1191 | LC CDR1 | SANSTVGYIH |
| SEQ ID NO: 1192 | LC CDR2 | TTSNLAS |
| SEQ ID NO: 1193 | LC CDR3 | HQWSFYPT |
| SEQ ID NO: 1196 | VH | QIQLQQSGPEVVKPGASVQISCKASGYTFSGSWMNWVKQRPG |
| | | KGLEWIGRIYPGDGDTDYSGKFKGRATLTADKSSSTAYMRLSS |
| | | LTSEDSAVYFCARSGYFNYVPVFDYWGQGTTLSVSS |
| SEQ ID NO: 1195 | VL | QIVLTQSPAIMSASLGEEIALTCSANSTVGYIHWYQQKSGTSPK |
| | | LLIYTTSNLASGVPSRFSGSGSGTFYSLTISSVEAEDAADYFCH |
| | | QWSFYPTFGGGTKLEIK |
| SEQ ID NO: 3649 | Heavy chain constant | ASTKGPSVFPLAPSSKSTSGGTAALGCLVKDYFPEPVTVSWNS |
| | region (CH1-CH2- | GALTSGVHTFPAVLQSSGLYSLSSVVTVPSSSLGTQTYICNVNH |
| | CH3) Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSPGK |
| SEQ ID NO: 3451 | Heavy chain constant | ASTKGPSVFPLAPSSKSTSGGTAALGCLVKDYFPEPVTVSWNS |
| | region (CH1-CH2- | GALTSGVHTFPAVLQSSGLYSLSSVVTVPSSSLGTQTYICNVNH |
| | CH3) Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSP |
| SEQ ID NO: 3447 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSPGK |
| SEQ ID NO: 511 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSP |
| SEQ ID NO: 3644 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 2270 | IL-2 C125A | APTSSSTKKTQLQLEHLLLDLQMILNGINNYKNPKLTRMLTFK |
| | | FYMPKKATELKHLQCLEEELKPLEEVLNLAQSKNFHLRPRDLI |
| | | SNINVIVLELKGSETTFMCEYADETATIVEFLNRWITFAQSIIST |
| | | LT |
| SEQ ID NO: 3308 | Linker | GGGGSGGGGS |
| SEQ ID NO: 3648 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |
| SEQ ID NO: 508 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSP |

TABLE 48
Exemplary molecule comprising an antigen binding domain that binds to a T cell
receptor beta variable region (TCRβV), wherein the exemplary antigen binding domain comprises
anti-TCRβV19 antibodies. Exemplary antigen binding domain comprises TCRVB 19. Exemplary
anti-TCRβV19 antibodies include, but are not limited to, BLM0281, which binds to human
TCRβV19. CDRs are underlined.
BLM0281, also referred to as BL195 or BJ1597
Binds to human TCRVβ 19

| | | |
|---|---|---|
| SEQ ID NO: 3368 | Heavy chain (VH- | NVQLQESGPGLVKPSQSLSLTCSVAGYSITSGYFWNWIRQFP |
| | CH) | GNKLEWMGYISYDGSNNYNPSLKNRISITRDTSKNQFFLKLN |
| | | SVTTEDTATYYCASPSPGTGYAVDYWGQGTSVTVSSASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKRVEPKSCDKTHTCPPCPAPELLGGPSVFLFPPKPKDTL |
| | | MISRTPEVTCVVVDVSHEDPEVKFNWYVDGVEVHNAKTKPR |
| | | EEQYASTYRVVSVLTVLHQDWLNGKEYKCKVSNKALPAPIE |
| | | KTISKAKGQPREPQVCTLPPSREEMTKNQVSLSCAVKGFYPS |
| | | DIAVEWESNGQPENNYKTTPPVLDSDGSFFLVSKLTVDKSRW |
| | | QQGNVFSCSVMHEALHNHYTQKSLSLSPGK |
| SEQ ID NO: 4713 | Heavy chain (VH- | NVQLQESGPGLVKPSQSLSLTCSVAGYSITSGYFWNWIRQFP |
| | CH) | GNKLEWMGYISYDGSNNYNPSLKNRISITRDTSKNQFFLKLN |
| | | SVTTEDTATYYCASPSPGTGYAVDYWGQGTSVTVSSASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKRVEPKSCDKTHTCPPCPAPELLGGPSVFLFPPKPKDTL |
| | | MISRTPEVTCVVVDVSHEDPEVKFNWYVDGVEVHNAKTKPR |
| | | EEQYASTYRVVSVLTVLHQDWLNGKEYKCKVSNKALPAPIE |
| | | KTISKAKGQPREPQVCTLPPSREEMTKNQVSLSCAVKGFYPS |
| | | DIAVEWESNGQPENNYKTTPPVLDSDGSFFLVSKLTVDKSRW |
| | | QQGNVFSCSVMHEALHNHYTQKSLSLSP |
| SEQ ID NO: 3369 | Light chain (VL-CL) | NVVMTQTPLSLPVSLGDQASISCRSSQSLVHSNGNTYLHWYL |
| | | QKPGQSPKFLIYKVSNRFSGVPDRFSGGGSGTEFTLKISRVEA |
| | | EDLGVYFCSQSTHVPFTFGSGTKLEIKRTVAAPSVFIFPPSDEQ |
| | | LKSGTASVVCLLNNFYPREAKVQWKVDNALQSGNSQESVTE |
| | | QDSKDSTYSLSSTLTLSKADYEKHKVYACEVTHQGLSSPVTK |
| | | SFNRGEC |
| SEQ ID NO: 3340 | IL-2 C125A-Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGSGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVYTLPPCREEMTKNQVSLWCLVKGFY |
| | | PSDIAVEWESNGQPENNYKTTPPVLDSDGSFFLYSKLTVDKS |
| | | RWQQGNVFSCSVMHEALHNHYTQKSLSLSPGK |
| SEQ ID NO: 4706 | IL-2 C125A-Heavy | APTSSSTKKTQLQLEHLLLDLQMILNGINNYKNPKLTRMLTF |
| | Chain Constant | KFYMPKKATELKHLQCLEEELKPLEEVLNLAQSKNFHLRPRD |
| | Domain (Fc Region) | LISNINVIVLELKGSETTFMCEYADETATIVEFLNRWITFAQSII |
| | | STLTGGGGSGGGGSDKTHTCPPCPAPELLGGPSVFLFPPKPKD |
| | | TLMISRTPEVTCVVVDVSHEDPEVKFNWYVDGVEVHNAKTK |
| | | PREEQYASTYRVVSVLTVLHQDWLNGKEYKCKVSNKALPAP |
| | | IEKTISKAKGQPREPQVYTLPPCREEMTKNQVSLWCLVKGFY |
| | | PSDIAVEWESNGQPENNYKTTPPVLDSDGSFFLYSKLTVDKS |
| | | RWQQGNVFSCSVMHEALHNHYTQKSLSLSP |
| SEQ ID NO: 3371 | HC CDR1 | SITSGYFWN |
| SEQ ID NO: 3372 | HC CDR2 | YISYDGSNNYNPSLKN |
| SEQ ID NO: 3373 | HC CDR3 | PSPGTGYAVDY |
| SEQ ID NO: 3374 | LC CDR1 | RSSQSLVHSNGNTYLH |
| SEQ ID NO: 3375 | LC CDR2 | KVSNRFS |
| SEQ ID NO: 3376 | LC CDR3 | SQSTHVPFT |
| SEQ ID NO: 3455 | VH | NVQLQESGPGLVKPSQSLSLTCSVAGYSITSGYFWNWIRQFPG |
| | | NKLEWMGYISYDGSNNYNPSLKNRISITRDTSKNQFFLKLNSV |
| | | TTEDTATYYCASPSPGTGYAVDYWGQGTSVTVSS |
| SEQ ID NO: 3456 | VL | NVVMTQTPLSLPVSLGDQASISCRSSQSLVHSNGNTYLHWYLQ |
| | | KPGQSPKFLIYKVSNRFSGVPDRFSGGGSGTEFTLKISRVEAED |
| | | LGVYFCSQSTHVPFTFGSGTKLEIK |
| SEQ ID NO: 3649 | Heavy chain constant | ASTKGPSVFPLAPSSKSTSGGTAALGCLVKDYFPEPVTVSWNS |
| | region (CH1-CH2- | GALTSGVHTFPAVLQSSGLYSLSSVVTVPSSSLGTQTYICNVNH |
| | CH3) Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSPGK |
| SEQ ID NO: 3451 | Heavy chain constant | ASTKGPSVFPLAPSSKSTSGGTAALGCLVKDYFPEPVTVSWNS |
| | region (CH1-CH2- | GALTSGVHTFPAVLQSSGLYSLSSVVTVPSSSLGTQTYICNVNH |
| | CH3) Hole | KPSNTKVDKRVEPKSCDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVCTLPPSREEMTKNQVSLSCAVKGFYP |
| | | SDIAVEWESNGQPENNYKTTPPVLDSDGSFFLVSKLTVDKSRW |
| | | QQGNVFSCSVMHEALHNHYTQKSLSLSP |
| SEQ ID NO: 3447 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSPGK |
| SEQ ID NO: 511 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | Hole | LTVLHQDWLNGKEYKCKVSNKALPAPIEKTISKAKGQPREPQ |
| | | VCTLPPSREEMTKNQVSLSCAVKGFYPSDIAVEWESNGQPENN |
| | | YKTTPPVLDSDGSFFLVSKLTVDKSRWQQGNVFSCSVMHEAL |
| | | HNHYTQKSLSLSP |
| SEQ ID NO: 3644 | Light chain constant | RTVAAPSVFIFPPSDEQLKSGTASVVCLLNNFYPREAKVQWKV |
| | region | DNALQSGNSQESVTEQDSKDSTYSLSSTLTLSKADYEKHKVY |
| | | ACEVTHQGLSSPVTKSFNRGEC |
| SEQ ID NO: 2270 | IL-2 C125A | APTSSSTKKTQLQLEHLLLDLQMILNGINNYKNPKLTRMLTFK |
| | | FYMPKKATELKHLQCLEEELKPLEEVLNLAQSKNFHLRPRDLI |
| | | SNINVIVLELKGSETTFMCEYADETATIVEFLNRWITFAQSIIST |
| | | LT |
| SEQ ID NO: 3308 | Linker | GGGGSGGGGS |
| SEQ ID NO: 3648 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSPGK |
| SEQ ID NO: 508 | Heavy chain hinge- | DKTHTCPPCPAPELLGGPSVFLFPPKPKDTLMISRTPEVTCVVV |
| | Fc region (CH2-CH3) | DVSHEDPEVKFNWYVDGVEVHNAKTKPREEQYASTYRVVSV |
| | knob | LTVLHQDWLNGKEYKCKVSNKALPAPIEKTISKAKGQPREPQ |
| | | VYTLPPCREEMTKNQVSLWCLVKGFYPSDIAVEWESNGQPEN |
| | | NYKTTPPVLDSDGSFFLYSKLTVDKSRWQQGNVFSCSVMHEA |
| | | LHNHYTQKSLSLSP |

TABLE 49
Exemplary molecule comprising an antigen binding domain that binds to a T cell
receptor beta variable region (TCRβV), wherein the exemplary antigen binding domain
comprises anti-TCRβ V17 antibodies. Exemplary antigen binding domain comprises TCRVB 17.
Exemplary anti-TCRβ V17 antibodies include, but are not limited to, BMM0034, which binds to
murine TCRβV 17. CDRs are underlined.
BMM0034, also referred to as BI432 or BL431
Binds to murine TCRVβ 17

| | | |
|---|---|---|
| SEQ ID NO: 3378 | Heavy chain (VH- | QVQLQQPGAELVRPGASVKLSCKASGYTFTNYWMNWVKQR |
| | CH) | PGQGLEWIGMIHPSDSATRLNQKFKDKATLTVDKSSSTAYMQ |
| | | LSSPTSEDSAVYYCARWAYYGNLYAIDYWGQGTSVTVSSAK |
| | | TTAPSVYPLAPVCGDTTGSSVTLGCLVKGYFPEPVTLTWNSG |
| | | SLSSGVHTFPAVLQSDLYTLSSSVTVTSSTWPSQSITCNVAHP |
| | | ASSTKVDKKIEPRGPTIKPCPPCKCPAPNAAGGPSVFIFPPKIK |
| | | DVLMISLSPIVTCVVVDVSEDDPDVQISWFVNNVEVHTAQTQ |
| | | THREDYNSTLRVVSALPIQHQDWMSGKEFKCKVNNKDLGAP |
| | | IERTISKPKGSVRAPQVCVLPPPEEEMTKKQVTLSCAVTDFMP |
| | | EDIYVEWTNNGKTELNYKNTEPVLDSDGSYFMVSKLRVEKK |
| | | NWVERNSYSCSVVHEGLHNHHTTKSFSRTPGK |
| SEQ ID NO: 4714 | Heavy chain (VH- | QVQLQQPGAELVRPGASVKLSCKASGYTFTNYWMNWVKQR |
| | CH) | PGQGLEWIGMIHPSDSATRLNQKFKDKATLTVDKSSSTAYMQ |
| | | LSSPTSEDSAVYYCARWAYYGNLYAIDYWGQGTSVTVSSAK |
| | | TTAPSVYPLAPVCGDTTGSSVTLGCLVKGYFPEPVTLTWNSG |
| | | SLSSGVHTFPAVLQSDLYTLSSSVTVTSSTWPSQSITCNVAHP |
| | | ASSTKVDKKIEPRGPTIKPCPPCKCPAPNAAGGPSVFIFPPKIK |
| | | DVLMISLSPIVTCVVVDVSEDDPDVQISWFVNNVEVHTAQTQ |
| | | THREDYNSTLRVVSALPIQHQDWMSGKEFKCKVNNKDLGAP |
| | | IERTISKPKGSVRAPQVCVLPPPEEEMTKKQVTLSCAVTDFMP |
| | | EDIYVEWTNNGKTELNYKNTEPVLDSDGSYFMVSKLRVEKK |
| | | NWVERNSYSCSVVHEGLHNHHTTKSFSRTP |
| SEQ ID NO: 3379 | Light chain (VL-CL) | DIVLTQSPASLAVSLGQRATISCKASQSVDYDGYSYMHWFQQ |
| | | KPGQPPKLLIYAASNLESGIPARFIGSGSGTDFTLNIHPVEEED |
| | | AATYYCQQSNEDPWTFGGGTKLEIKRADAAPTVSIFPPSSEQL |
| | | TSGGASVVCFLNNFYPKDINVKWKIDGSERQNGVLNSWTDQ |
| | | DSKDSTYSMSSTLTLTKDEYERHNSYTCEATHKTSTSPIVKSF |
| | | NRNEC |
| SEQ ID NO: 3380 | IL-2-Heavy Chain | APTSSSTKKTQLQLEHLLLDLQMILNGINNYKNPKLTRMLTF |
| | Constant Domain (Fc | KFYMPKKATELKHLQCLEEELKPLEEVLNLAQSKNFHLRPRD |
| | Region) | LISNINVIVLELKGSETTFMCEYADETATIVEFLNRWITFCQSII |
| | | STLTGGGGSGGGGSTIKPCPPCKCPAPNAAGGPSVFIFPPKIKD |
| | | VLMISLSPIVTCVVVDVSEDDPDVQISWFVNNVEVHTAQTQT |
| | | HREDYNSTLRVVSALPIQHQDWMSGKEFKCKVNNKDLGAPI |
| | | ERTISKPKGSVRAPQVYVLPPCEEEMTKKQVTLWCMVTDFM |
| | | PEDIYVEWTNNGKTELNYKNTEPVLDSDGSYFMYSKLRVEK |
| | | KNWVERNSYSCSVVHEGLHNHHTTKSFSRTPGK |
| SEQ ID NO: 4715 | IL-2-Heavy Chain | APTSSSTKKTQLQLEHLLLDLQMILNGINNYKNPKLTRMLTF |
| | Constant Domain (Fc | KFYMPKKATELKHLQCLEEELKPLEEVLNLAQSKNFHLRPRD |
| | Region) | LISNINVIVLELKGSETTFMCEYADETATIVEFLNRWITFCQSII |
| | | STLTGGGGSGGGGSTIKPCPPCKCPAPNAAGGPSVFIFPPKIKD |
| | | VLMISLSPIVTCVVVDVSEDDPDVQISWFVNNVEVHTAQTQT |
| | | HREDYNSTLRVVSALPIQHQDWMSGKEFKCKVNNKDLGAPI |
| | | ERTISKPKGSVRAPQVYVLPPCEEEMTKKQVTLWCMVTDFM |
| | | PEDIYVEWTNNGKTELNYKNTEPVLDSDGSYFMYSKLRVEK |
| | | KNWVERNSYSCSVVHEGLHNHHTTKSFSRTP |
| SEQ ID NO: 3381 | HC CDR1 | GYTFTNYWMN |
| SEQ ID NO: 3382 | HC CDR2 | MIHPSDSATRLNQKFKD |
| SEQ ID NO: 3383 | HC CDR3 | WAYYGNLYAIDY |
| SEQ ID NO: 3384 | LC CDR1 | KASQSVDYDGYSYMH |
| SEQ ID NO: 3325 | LC CDR2 | AASNLES |
| SEQ ID NO: 3386 | LC CDR3 | QQSNEDPWT |
| SEQ ID NO: 3457 | VH | QVQLQQPGAELVRPGASVKLSCKASGYTFTNYWMNWVKQRP |
| | | GQGLEWIGMIHPSDSATRLNQKFKDKATLTVDKSSSTAYMQL |
| | | SSPTSEDSAVYYCARWAYYGNLYAIDYWGQGTSVTVSS |
| SEQ ID NO: 3458 | VL | DIVLTQSPASLAVSLGQRATISCKASQSVDYDGYSYMHWFQQ |
| | | KPGQPPKLLIYAASNLESGIPARFIGSGSGTDFTLNIHPVEEEDA |
| | | ATYYCQQSNEDPWTFGGGTKLEIK |
| SEQ ID NO: 3459 | Heavy chain constant | AKTTAPSVYPLAPVCGDTTGSSVTLGCLVKGYFPEPVTLTWNS |
| | region (CH1-CH2- | GSLSSGVHTFPAVLQSDLYTLSSSVTVTSSTWPSQSITCNVAHP |
| | CH3) Hole | ASSTKVDKKIEPRGPTIKPCPPCKCPAPNAAGGPSVFIFPPKIKD |
| | | VLMISLSPIVTCVVVDVSEDDPDVQISWFVNNVEVHTAQTQTH |
| | | REDYNSTLRVVSALPIQHQDWMSGKEFKCKVNNKDLGAPIER |
| | | TISKPKGSVRAPQVCVLPPPEEEMTKKQVTLSCAVTDFMPEDI |
| | | YVEWTNNGKTELNYKNTEPVLDSDGSYFMVSKLRVEKKNWV |
| | | ERNSYSCSVVHEGLHNHHTTKSFSRTPGK |
| SEQ ID NO: 4716 | Heavy chain constant | AKTTAPSVYPLAPVCGDTTGSSVTLGCLVKGYFPEPVTLTWNS |
| | region (CH1-CH2- | GSLSSGVHTFPAVLQSDLYTLSSSVTVTSSTWPSQSITCNVAHP |
| | CH3) Hole | ASSTKVDKKIEPRGPTIKPCPPCKCPAPNAAGGPSVFIFPPKIKD |
| | | VLMISLSPIVTCVVVDVSEDDPDVQISWFVNNVEVHTAQTQTH |
| | | REDYNSTLRVVSALPIQHQDWMSGKEFKCKVNNKDLGAPIER |
| | | TISKPKGSVRAPQVCVLPPPEEEMTKKQVTLSCAVTDFMPEDI |
| | | YVEWTNNGKTELNYKNTEPVLDSDGSYFMVSKLRVEKKNWV |
| | | ERNSYSCSVVHEGLHNHHTTKSFSRTP |
| SEQ ID NO: 3460 | Heavy chain hinge- | TIKPCPPCKCPAPNAAGGPSVFIFPPKIKDVLMISLSPIVTCVVV |
| | Fc region (CH2-CH3) | DVSEDDPDVQISWFVNNVEVHTAQTQTHREDYNSTLRVVSAL |
| | Hole | PIQHQDWMSGKEFKCKVNNKDLGAPIERTISKPKGSVRAPQVC |
| | | VLPPPEEEMTKKQVTLSCAVTDFMPEDIYVEWTNNGKTELNY |
| | | KNTEPVLDSDGSYFMVSKLRVEKKNWVERNSYSCSVVHEGL |
| | | HNHHTTKSFSRTPGK |
| SEQ ID NO: 4717 | Heavy chain hinge- | TIKPCPPCKCPAPNAAGGPSVFIFPPKIKDVLMISLSPIVTCVVV |
| | Fc region (CH2-CH3) | DVSEDDPDVQISWFVNNVEVHTAQTQTHREDYNSTLRVVSAL |
| | Hole | PIQHQDWMSGKEFKCKVNNKDLGAPIERTISKPKGSVRAPQVC |
| | | VLPPPEEEMTKKQVTLSCAVTDFMPEDIYVEWTNNGKTELNY |
| | | KNTEPVLDSDGSYFMVSKLRVEKKNWVERNSYSCSVVHEGL |
| | | HNHHTTKSFSRTP |
| SEQ ID NO: 3461 | Light chain constant | RADAAPTVSIFPPSSEQLTSGGASVVCFLNNFYPKDINVKWKID |
| | region | GSERQNGVLNSWTDQDSKDSTYSMSSTLTLTKDEYERHNSYT |
| | | CEATHKTSTSPIVKSFNRNEC |
| SEQ ID NO: 2191 | IL-2 | APTSSSTKKTQLQLEHLLLDLQMILNGINNYKNPKLTRMLTFK |
| | | FYMPKKATELKHLQCLEEELKPLEEVLNLAQSKNFHLRPRDLI |
| | | SNINVIVLELKGSETTFMCEYADETATIVEFLNRWITFCQSIISTL |
| | | T |
| SEQ ID NO: 3308 | Linker | GGGGSGGGGS |
| SEQ ID NO: 3462 | Heavy chain hinge- | TIKPCPPCKCPAPNAAGGPSVFIFPPKIKDVLMISLSPIVTCVVV |
| | Fc region (CH2-CH3) | DVSEDDPDVQISWFVNNVEVHTAQTQTHREDYNSTLRVVSAL |
| | knob | PIQHQDWMSGKEFKCKVNNKDLGAPIERTISKPKGSVRAPQV |
| | | YVLPPCEEEMTKKQVTLWCMVTDFMPEDIYVEWTNNGKTEL |
| | | NYKNTEPVLDSDGSYFMYSKLRVEKKNWVERNSYSCSVVHE |
| | | GLHNHHTTKSFSRTPGK |
| SEQ ID NO: 4718 | Heavy chain hinge- | TIKPCPPCKCPAPNAAGGPSVFIFPPKIKDVLMISLSPIVTCVVV |
| | Fc region (CH2-CH3) | DVSEDDPDVQISWFVNNVEVHTAQTQTHREDYNSTLRVVSAL |
| | knob | PIQHQDWMSGKEFKCKVNNKDLGAPIERTISKPKGSVRAPQV |
| | | YVLPPCEEEMTKKQVTLWCMVTDFMPEDIYVEWTNNGKTEL |
| | | NYKNTEPVLDSDGSYFMYSKLRVEKKNWVERNSYSCSVVHE |
| | | GLHNHHTTKSFSRTP |

TABLE 50
Exemplary molecule comprising an antigen binding domain that binds to a T cell
receptor beta variable region (TCRβV), wherein the exemplary antigen binding domain comprises
anti-TCRβ V29 antibodies. Exemplary antigen binding domain comprises TCRVB 29. Exemplary
anti-TCRβ V29 antibodies include, but are not limited to, BMM0019, which binds to murine
TCRβV 29. CDRs are underlined.
BMM0019, also referred to as BL404 or BL405
Binds to murine TCRVβ 29

| | | |
|---|---|---|
| SEQ ID NO: 3388 | Heavy chain (VH- | EVQLVESGGGLVQPGASLKLSCVASGFTFSDYWMSWVRQTP |
| | CH) | GKTMEWIGNIKYDGSHTNYAPSLKNRFTISRDNAKSTLFLQM |
| | | SNVRSEDTATYYCTRESHYGYIYPFDYWGQGVMVTVSSAKT |
| | | TAPSVYPLAPVCGDTTGSSVTLGCLVKGYFPEPVTLTWNSGS |
| | | LSSGVHTFPAVLQSDLYTLSSSVTVTSSTWPSQSITCNVAHPA |
| | | SSTKVDKKIEPRGPTIKPCPPCKCPAPNAAGGPSVFIFPPKIKD |
| | | VLMISLSPIVTCVVVDVSEDDPDVQISWFVNNVEVHTAQTQT |
| | | HREDYNSTLRVVSALPIQHQDWMSGKEFKCKVNNKDLGAPI |
| | | ERTISKPKGSVRAPQVCVLPPPEEEMTKKQVTLSCAVTDFMP |
| | | EDIYVEWTNNGKTELNYKNTEPVLDSDGSYFMVSKLRVEKK |
| | | NWVERNSYSCSVVHEGLHNHHTTKSFSRTPGK |
| SEQ ID NO: 4719 | Heavy chain (VH- | EVQLVESGGGLVQPGASLKLSCVASGFTFSDYWMSWVRQTP |
| | CH) | GKTMEWIGNIKYDGSHTNYAPSLKNRFTISRDNAKSTLFLQM |
| | | SNVRSEDTATYYCTRESHYGYIYPFDYWGQGVMVTVSSAKT |
| | | TAPSVYPLAPVCGDTTGSSVTLGCLVKGYFPEPVTLTWNSGS |
| | | LSSGVHTFPAVLQSDLYTLSSSVTVTSSTWPSQSITCNVAHPA |
| | | SSTKVDKKIEPRGPTIKPCPPCKCPAPNAAGGPSVFIFPPKIKD |
| | | VLMISLSPIVTCVVVDVSEDDPDVQISWFVNNVEVHTAQTQT |
| | | HREDYNSTLRVVSALPIQHQDWMSGKEFKCKVNNKDLGAPI |
| | | ERTISKPKGSVRAPQVCVLPPPEEEMTKKQVTLSCAVTDFMP |
| | | EDIYVEWTNNGKTELNYKNTEPVLDSDGSYFMVSKLRVEKK |
| | | NWVERNSYSCSVVHEGLHNHHTTKSFSRTP |
| SEQ ID NO: 3389 | Light chain (VL-CL) | DIQMTQSPSSMSVSLGDTVTITCRASQDVGIYVNWFQQKPGK |
| | | SPRRMIYRATNLADGVPSRFSGSRSGSDYSLTISSLESEDVAD |
| | | YHCLQYDESPFTFGSGTKLEIKRADAAPTVSIFPPSSEQLTSGG |
| | | ASVVCFLNNFYPKDINVKWKIDGSERQNGVLNSWTDQDSKD |
| | | STYSMSSTLTLTKDEYERHNSYTCEATHKTSTSPIVKSFNRNE |
| | | C |
| SEQ ID NO: 3390 | IL-2-Heavy Chain | APTSSSTKKTQLQLEHLLLDLQMILNGINNYKNPKLTRMLTF |
| | Constant Domain (Fc | KFYMPKKATELKHLQCLEEELKPLEEVLNLAQSKNFHLRPRD |
| | Region) | LISNINVIVLELKGSETTFMCEYADETATIVEFLNRWITFCQSII |
| | | STLTGGGGSGGGGSTIKPCPPCKCPAPNAAGGPSVFIFPPKIKD |
| | | VLMISLSPIVTCVVVDVSEDDPDVQISWFVNNVEVHTAQTQT |
| | | HREDYNSTLRVVSALPIQHQDWMSGKEFKCKVNNKDLGAPI |
| | | ERTISKPKGSVRAPQVYVLPPCEEEMTKKQVTLWCMVTDFM |
| | | PEDIYVEWTNNGKTELNYKNTEPVLDSDGSYFMYSKLRVEK |
| | | KNWVERNSYSCSVVHEGLHNHHTTKSFSRTPGK |
| SEQ ID NO: 4720 | IL-2-Heavy Chain | APTSSSTKKTQLQLEHLLLDLQMILNGINNYKNPKLTRMLTF |
| | Constant Domain (Fc | KFYMPKKATELKHLQCLEEELKPLEEVLNLAQSKNFHLRPRD |
| | Region) | LISNINVIVLELKGSETTFMCEYADETATIVEFLNRWITFCQSII |
| | | STLTGGGGSGGGGSTIKPCPPCKCPAPNAAGGPSVFIFPPKIKD |
| | | VLMISLSPIVTCVVVDVSEDDPDVQISWFVNNVEVHTAQTQT |
| | | HREDYNSTLRVVSALPIQHQDWMSGKEFKCKVNNKDLGAPI |
| | | ERTISKPKGSVRAPQVYVLPPCEEEMTKKQVTLWCMVTDFM |
| | | PEDIYVEWTNNGKTELNYKNTEPVLDSDGSYFMYSKLRVEK |
| | | KNWVERNSYSCSVVHEGLHNHHTTKSFSRTP |
| SEQ ID NO: 3391 | HC CDR1 | GFTFSDYWMS |
| SEQ ID NO: 3392 | HC CDR2 | NIKYDGSHTNYAPSLKN |
| SEQ ID NO: 3393 | HC CDR3 | ESHYGYIYPFDY |
| SEQ ID NO: 3394 | LC CDR1 | RASQDVGIYVN |
| SEQ ID NO: 3395 | LC CDR2 | RATNLAD |
| SEQ ID NO: 3396 | LC CDR3 | LQYDESPFT |
| SEQ ID NO: 3463 | VH | EVQLVESGGGLVQPGASLKLSCVASGFTFSDYWMSWVRQTPG |
| | | KTMEWIGNIKYDGSHTNYAPSLKNRFTISRDNAKSTLFLQMSN |
| | | VRSEDTATYYCTRESHYGYIYPFDYWGQGVMVTVSS |
| SEQ ID NO: 3464 | VL | DIQMTQSPSSMSVSLGDTVTITCRASQDVGIYVNWFQQKPGKS |
| | | PRRMIYRATNLADGVPSRFSGSRSGSDYSLTISSLESEDVADYH |
| | | CLQYDESPFTFGSGTKLEIK |
| SEQ ID NO: 3459 | Heavy chain constant | AKTTAPSVYPLAPVCGDTTGSSVTLGCLVKGYFPEPVTLTWNS |
| | region (CH1-CH2- | GSLSSGVHTFPAVLQSDLYTLSSSVTVTSSTWPSQSITCNVAHP |
| | CH3) Hole | ASSTKVDKKIEPRGPTIKPCPPCKCPAPNAAGGPSVFIFPPKIKD |
| | | VLMISLSPIVTCVVVDVSEDDPDVQISWFVNNVEVHTAQTQTH |
| | | REDYNSTLRVVSALPIQHQDWMSGKEFKCKVNNKDLGAPIER |
| | | TISKPKGSVRAPQVCVLPPPEEEMTKKQVTLSCAVTDFMPEDI |
| | | YVEWTNNGKTELNYKNTEPVLDSDGSYFMVSKLRVEKKNWV |
| | | ERNSYSCSVVHEGLHNHHTTKSFSRTPGK |
| SEQ ID NO: 4716 | Heavy chain constant | AKTTAPSVYPLAPVCGDTTGSSVTLGCLVKGYFPEPVTLTWNS |
| | region (CH1-CH2- | GSLSSGVHTFPAVLQSDLYTLSSSVTVTSSTWPSQSITCNVAHP |
| | CH3) Hole | ASSTKVDKKIEPRGPTIKPCPPCKCPAPNAAGGPSVFIFPPKIKD |
| | | VLMISLSPIVTCVVVDVSEDDPDVQISWFVNNVEVHTAQTQTH |
| | | REDYNSTLRVVSALPIQHQDWMSGKEFKCKVNNKDLGAPIER |
| | | TISKPKGSVRAPQVCVLPPPEEEMTKKQVTLSCAVTDFMPEDI |
| | | YVEWTNNGKTELNYKNTEPVLDSDGSYFMVSKLRVEKKNWV |
| | | ERNSYSCSVVHEGLHNHHTTKSFSRTP |
| SEQ ID NO: 3460 | Heavy chain hinge- | TIKPCPPCKCPAPNAAGGPSVFIFPPKIKDVLMISLSPIVTCVVV |
| | Fc region (CH2-CH3) | DVSEDDPDVQISWFVNNVEVHTAQTQTHREDYNSTLRVVSAL |
| | Hole | PIQHQDWMSGKEFKCKVNNKDLGAPIERTISKPKGSVRAPQVC |
| | | VLPPPEEEMTKKQVTLSCAVTDFMPEDIYVEWTNNGKTELNY |
| | | KNTEPVLDSDGSYFMVSKLRVEKKNWVERNSYSCSVVHEGL |
| | | HNHHTTKSFSRTPGK |
| SEQ ID NO: 3461 | Light chain constant | RADAAPTVSIFPPSSEQLTSGGASVVCFLNNFYPKDINVKWKID |
| | region | GSERQNGVLNSWTDQDSKDSTYSMSSTLTLTKDEYERHNSYT |
| | | CEATHKTSTSPIVKSFNRNEC |
| SEQ ID NO: 2191 | IL-2 | APTSSSTKKTQLQLEHLLLDLQMILNGINNYKNPKLTRMLTFK |
| | | FYMPKKATELKHLQCLEEELKPLEEVLNLAQSKNFHLRPRDLI |
| | | SNINVIVLELKGSETTFMCEYADETATIVEFLNRWITFCQSIISTL |
| | | T |
| SEQ ID NO: 3308 | Linker | GGGGSGGGGS |
| SEQ ID NO: 3462 | Heavy chain hinge- | TIKPCPPCKCPAPNAAGGPSVFIFPPKIKDVLMISLSPIVTCVVV |
| | Fc region (CH2-CH3) | DVSEDDPDVQISWFVNNVEVHTAQTQTHREDYNSTLRVVSAL |
| | knob | PIQHQDWMSGKEFKCKVNNKDLGAPIERTISKPKGSVRAPQV |
| | | YVLPPCEEEMTKKQVTLWCMVTDFMPEDIYVEWTNNGKTEL |
| | | NYKNTEPVLDSDGSYFMYSKLRVEKKNWVERNSYSCSVVHE |
| | | GLHNHHTTKSFSRTPGK |
| SEQ ID NO: 4718 | Heavy chain hinge- | TIKPCPPCKCPAPNAAGGPSVFIFPPKIKDVLMISLSPIVTCVVV |
| | Fc region (CH2-CH3) | DVSEDDPDVQISWFVNNVEVHTAQTQTHREDYNSTLRVVSAL |
| | knob | PIQHQDWMSGKEFKCKVNNKDLGAPIERTISKPKGSVRAPQV |
| | | YVLPPCEEEMTKKQVTLWCMVTDFMPEDIYVEWTNNGKTEL |
| | | NYKNTEPVLDSDGSYFMYSKLRVEKKNWVERNSYSCSVVHE |
| | | GLHNHHTTKSFSRTP |

TABLE 51
Exemplary molecule comprising an antigen binding domain that binds to a T cell
receptor beta variable region (TCRβV), wherein the exemplary antigen binding domain comprises
anti-TCRβ V13 antibodies. Exemplary antigen binding domain comprises TCRVB 13 (e.g.,
TCRVβ 13-3). Exemplary anti-TCRβ V13 antibodies include, but are not limited to, BKM0307,
which binds to murine TCRBV 13-3. CDRs are underlined.
BKM0307, also referred to as BK057 or BJ1039
Binds to murine TCRVβ 13-3

| | | |
|---|---|---|
| SEQ ID NO: 3398 | Heavy chain (VH- | QVQLQQSGTELMKPGASVKISCKASGYTFSNYWIEWIKQRPG |
| | CH) | HGLEWVGEILPGAGPTNYNEKFKGKATFTADSSSNTAYMQL |
| | | SSLTSEDSAVYYCARTDYDYDWFAYWGQGTLVTVSAAKTT |
| | | APSVYPLAPVSGDTTGSSVTLGCLVKGYFPEPVTLTWNSGSL |
| | | SSGVHTFPAVLQSDLYTLSSSVTVTSSTWPSQSITCNVAHPAS |
| | | STKVDKKIEPKSCDKTHTCPPCPAPNAAGGPSVFIFPPKIKDVL |
| | | MISLSPIVTCVVVDVSEDDPDVQISWFVNNVEVHTAQTQTHR |
| | | EDYNSTLRVVSALPIQHQDWMSGKEFKCKVNNKDLGAPIER |
| | | TISKPKGSVRAPQVCVLPPPEEEMTKKQVTLSCAVTDFMPEDI |
| | | YVEWTNNGKTELNYKNTEPVLDSDGSYFMVSKLRVEKKNW |
| | | VERNSYSCSVVHEGLHNHHTTKSFSRTPGKEPEA |
| SEQ ID NO: 3399 | Light chain (VL-CL) | DIVMSQSPSSLAVSVGEKVTMSCKSSQSLLYSGNQKNYLAW |
| | | YQQKPGQSPKLLIYWASTRESGVPDRFTGSGSGTDFTLTINSV |
| | | KAEDLTVYYCQQYYGYPRTFGGGTKVEIKRADAAPTVSIFPP |
| | | SSEQLTSGGASVVCFLNNFYPKDINVKWKIDGSERQNGVLNS |
| | | WTDQDSKDSTYSMSSTLTLTKDEYERHNSYTCEATHKTSTSP |
| | | IVKSFNRNEC |
| SEQ ID NO: 3400 | IL-2-Heavy Chain | APTSSSTKKTQLQLEHLLLDLQMILNGINNYKNPKLTRMLTF |
| | Constant Domain (Fc | KFYMPKKATELKHLQCLEEELKPLEEVLNLAQSKNFHLRPRD |
| | Region) | LISNINVIVLELKGSETTFMCEYADETATIVEFLNRWITFCQSII |
| | | STLTGGGGSGGGGSDKTHTCPPCPAPNAAGGPSVFIFPPKIKD |
| | | VLMISLSPIVTCVVVDVSEDDPDVQISWFVNNVEVHTAQTQT |
| | | HREDYNSTLRVVSALPIQHQDWMSGKEFKCKVNNKDLGAPI |
| | | ERTISKPKGSVRAPQVYVLPPCEEEMTKKQVTLWCMVTDFM |
| | | PEDIYVEWTNNGKTELNYKNTEPVLDSDGSYFMYSKLRVEK |
| | | KNWVERNSYSCSVVHEGLHNHHTTKSFSRTPGKGGGGSHHH |
| | | HHHHH |
| SEQ ID NO: 3401 | HC CDR1 | GYTFSNYWIE |
| SEQ ID NO: 3402 | HC CDR2 | EILPGAGPTNYNEKFKG |
| SEQ ID NO: 3403 | HC CDR3 | TDYDYDWFAY |
| SEQ ID NO: 3404 | LC CDR1 | KSSQSLLYSGNQKNYLA |
| SEQ ID NO: 3405 | LC CDR2 | WASTRES |
| SEQ ID NO: 3406 | LC CDR3 | QQYYGYPRT |
| SEQ ID NO: 3465 | VH | QVQLQQSGTELMKPGASVKISCKASGYTFSNYWIEWIKQRPG |
| | | HGLEWVGEILPGAGPTNYNEKFKGKATFTADSSSNTAYMQLS |
| | | SLTSEDSAVYYCARTDYDYDWFAYWGQGTLVTVSA |
| SEQ ID NO: 3466 | VL | DIVMSQSPSSLAVSVGEKVTMSCKSSQSLLYSGNQKNYLAWY |
| | | QQKPGQSPKLLIYWASTRESGVPDRFTGSGSGTDFTLTINSVKA |
| | | EDLTVYYCQQYYGYPRTFGGGTKVEIK |
| SEQ ID NO: 3467 | Heavy chain constant | AKTTAPSVYPLAPVSGDTTGSSVTLGCLVKGYFPEPVTLTWNS |
| | region (CH1-CH2- | GSLSSGVHTFPAVLQSDLYTLSSSVTVTSSTWPSQSITCNVAHP |
| | CH3) Hole | ASSTKVDKKIEPKSCDKTHTCPPCPAPNAAGGPSVFIFPPKIKD |
| | | VLMISLSPIVTCVVVDVSEDDPDVQISWFVNNVEVHTAQTQTH |
| | | REDYNSTLRVVSALPIQHQDWMSGKEFKCKVNNKDLGAPIER |
| | | TISKPKGSVRAPQVCVLPPPEEEMTKKQVTLSCAVTDFMPEDI |
| | | YVEWTNNGKTELNYKNTEPVLDSDGSYFMVSKLRVEKKNWV |
| | | ERNSYSCSVVHEGLHNHHTTKSFSRTPGKEPEA |
| SEQ ID NO: 3468 | Heavy chain hinge- | DKTHTCPPCPAPNAAGGPSVFIFPPKIKDVLMISLSPIVTCVVVD |
| | Fc region (CH2-CH3) | VSEDDPDVQISWFVNNVEVHTAQTQTHREDYNSTLRVVSALPI |
| | Hole | QHQDWMSGKEFKCKVNNKDLGAPIERTISKPKGSVRAPQVCV |
| | | LPPPEEEMTKKQVTLSCAVTDFMPEDIYVEWTNNGKTELNYK |
| | | NTEPVLDSDGSYFMVSKLRVEKKNWVERNSYSCSVVHEGLH |
| | | NHHTTKSFSRTPGKEPEA |
| SEQ ID NO: 3461 | Light chain constant | RADAAPTVSIFPPSSEQLTSGGASVVCFLNNFYPKDINVKWKID |
| | region | GSERQNGVLNSWTDQDSKDSTYSMSSTLTLTKDEYERHNSYT |
| | | CEATHKTSTSPIVKSFNRNEC |
| SEQ ID NO: 2191 | IL-2 | APTSSSTKKTQLQLEHLLLDLQMILNGINNYKNPKLTRMLTFK |
| | | FYMPKKATELKHLQCLEEELKPLEEVLNLAQSKNFHLRPRDLI |
| | | SNINVIVLELKGSETTFMCEYADETATIVEFLNRWITFCQSIISTL |
| | | T |
| SEQ ID NO: 3308 | Linker | GGGGSGGGGS |
| SEQ ID NO: 3469 | Heavy chain hinge- | DKTHTCPPCPAPNAAGGPSVFIFPPKIKDVLMISLSPIVTCVVVD |
| | Fc region (CH2-CH3) | VSEDDPDVQISWFVNNVEVHTAQTQTHREDYNSTLRVVSALPI |
| | knob | QHQDWMSGKEFKCKVNNKDLGAPIERTISKPKGSVRAPQVYV |
| | | LPPCEEEMTKKQVTLWCMVTDFMPEDIYVEWTNNGKTELNY |
| | | KNTEPVLDSDGSYFMYSKLRVEKKNWVERNSYSCSVVHEGL |
| | | HNHHTTKSFSRTPGKGGGGSHHHHHHHH |

TABLE 52
TCRαV- and TCRβV-and related diseases or conditions

| Subfamily | Related conditions or diseases |
|---|---|
| TCRαV1 | multiple sclerosis |
| (including | |
| TCRαV1-1 | |
| and TCRα1-2) | |
| TCRαV2 | Crohn's disease |
| TCRαV4 | Celiac disease |
| TCRαV12 | Type I diabetes |
| (TCRαV12-3) | |
| TCRαV13 | Sjogren's syndrome |
| (including | |
| TCRαV13-1, | |
| TCRαV13-2) | |
| TCRαV20 | Celiac disease |
| TCRαV21 | ankylosing spondylitis |
| TCRαV22 | Crohn's disease |
| TCRαV26 | Celiac disease |
| (including | |
| TCRαV26-1, and | |
| TCRαV26-2) | |
| TCRαV40 | Crohn's disease |
| TCRαV41 | Joint implant failure |
| TCRβV4 | Celiac disease |
| TCRβV6 | Primary Sjogren's syndrome |
| (including | |
| TCRβV6-1, | |
| TCRβV6-2/3, | |
| and TCRβV6-5) | |
| TCRβV7 | Amyotrophic lateral sclerosis; |
| (including | celiac disease; multiple sclerosis |
| TCRβV7-1 and | |
| TCRβV7-8/9) | |
| TCRβV9 | Ankylosing spondylitis |
| TCRβV10 | Multiple sclerosis |
| (including | |
| TCRβV10-3) | |
| TCRβV11 | Covid-induced multisystem |
| (including | inflammatory syndrome in children |
| TCRβV11-2) | |
| TCRβV12 | Type I diabetes |
| (including | |
| TCRβV12-3/4) | |
| TCRβV20 | Multiple sclerosis |
| (including | |
| TCRβV20-1) | |
| TCRβV23 | Amyotrophic lateral sclerosis |
| TCRβV29 | Multiple sclerosis |
| (including | |
| TCRβV29-1) | |

References:
He et al., (2012) Neoplasma. 59(6): 693-9
Howson et al. (2018) Nat. Commun. 9, 253
Tastan et al. (2018) Mucosal Immunol. 11, 1591-1605
Carnero et al., (2019) Front. Immunol. 10, 2690
Huang et al. (2019) Proc. Natl. Acad. Sci. U.S.A. 116, 8995-9001
Hong et al., (2022) Front Cell Infect Microbiol. 10; 12:932373
Meermeier et al. (2016) Nat. Commun. 7, 12506
Yang et al., J Biol Chem. 2017 Nov. 10; 292(45): 18618-18627.
Surman et al., J Immunol. 2011 Jul. 15; 187(2): 835-41.
Motozono et al., 2014 Apr. 1; 192(7): 3428-34.
Brennan et al., (2007) J Virol. (13): 7269-73.
Huda et al., (2021) Leuk Lymphoma. 62(7): 1711-1720.
Petersen et al., 2016 Oct. 4; 24(10): 1643-1657.
Benati et al., J Clin Invest. 2016 Jun. 1; 126(6): 2093-108.
Frick et al., (2020) Eur J Immunol. 50(1): 142-145.
Valkenburg et al., (2016) Proc Natl Acad Sci U.S.A. 113(16): 4440-5.
Lepore et al., (2017) Elife 6, e24476
Zhou et al., (2020) Cancer Cell Int. 20: 240.
Rowntree et al., (2020) J immunol; 205(6), 1524-1534
Crowther et al., (2020) Nat. Immunol. 21, 178-185
Xu et al., (2022) J Oncol. 3152114
Chen et al., (2021) Int J Mol Sci. 22(5): 2428
Bovay et al., (2018) Eur J Immunol. 48(2): 258-272.
Godfrey et al., (2015) Nat Immunol. 16(11): 1114-23.
Cole et al., (2009) J Biol Chem. 284(40): 27281-9.
Szeto et al., (2021) Cells. 2021 Oct. 3; 10(10): 2646.
Campisi et al., (2022) Nature. 606: 945-952.
Koirala et al., (2016) Mucosal Immunology; 9(3): 587-596.
Yohannes et al., (2017) Sci. Rep.; 7(17977).
Porritt et al., (2021) J Clin Invest.; 131(10): e146614.
Lu et al., (2022) eBioMedicine; 84: 104252.
Helliwell et al., (2005) Ann Rheum Dis; 64: 468-470.
Lossius et al., (2014) Eur. J. Immunol.; 44: 3439-3452.
Bulek et al., (2012) Nature Immunology; 13(3): 283-289.
Tan et al., (2010) Hematology, 15: 2, 81-87
Gedda et al., (2022) J Transl Med. 20(1): 587

What is claimed is:

1.-263. (canceled)

264. A composition comprising an agent that comprises an antigen binding domain that binds to NKp30, wherein the antigen binding domain comprises:
(a) a heavy chain variable region (VH) comprising a heavy chain complementarity determining region 1 (VHCDR1) comprising the sequence of GFSIIRVGYHWN (SEQ ID NO: 502), a heavy chain complementarity determining region 2 (VHCDR2) comprising the sequence of YIHRSGSTSYNPSLKS (SEQ ID NO: 503), and a heavy chain complementarity determining region 3 (VHCDR3) comprising the sequence of GDWHYFDY (SEQ ID NO: 504), and
(b) a light chain variable region (VL) comprising a light chain complementarity determining region 1 (VLCDR1) comprising the sequence of SGEKLSDKYVH (SEQ ID NO: 26), a light chain complementarity determining region 2 (VLCDR2) comprising the sequence of ENDRRPS (SEQ ID NO: 28), a light chain complementarity determining region 3 (VLCDR3) comprising the sequence of QFWDSTNSAV (SEQ ID NO: 86).

265. The composition of claim 264, wherein the antigen binding domain comprises a VH comprising the sequence of SEQ ID NO: 505.

266. The composition of claim 264, wherein the antigen binding domain comprises a VL comprising the sequence of SEQ ID NO: 13.

267. The composition of claim 264, wherein the agent further comprises a cytokine molecule or a functional fragment or variant thereof.

268. The composition of claim 267, wherein the cytokine molecule or a functional fragment or variant thereof comprises IL-2 or a functional fragment or variant thereof, IL-7 or a functional fragment or variant thereof, IL-12 or a functional fragment or variant thereof, IL-15 or a functional fragment or variant thereof, IL-18 or a functional fragment or variant thereof, IL-21 or a functional fragment or variant thereof, interferon gamma or a functional fragment or variant thereof, or any combination thereof.

269. The composition of claim 268, wherein the cytokine molecule or a functional fragment or variant thereof comprises IL-15 or a functional fragment or variant thereof.

270. The composition of claim 264, wherein the agent further comprises an antigen binding domain that binds to TRBC.

271. The composition of claim 270, wherein the antigen binding domain that binds to TRBC binds to TRBC1 or TRBC2.

272. The composition of claim 264, wherein the agent further comprises an anti-T cell receptor beta chain variable region (TCRβV) antibody molecule.

273. The composition of claim 272, wherein the anti-TCRβV antibody molecule is an anti-TRBV9.

274. The composition of claim 272, wherein the anti-TCRβV antibody molecule is an anti-TRBV5.

275. The composition of claim 274, wherein the anti-TRBV5 comprises:
(a) a heavy chain variable region (VH) comprising a heavy chain complementarity determining region 1 (VHCDR1) comprising the sequence of GYTFTDYNIH (SEQ ID NO: 3341), a heavy chain complementarity determining region 2 (VHCDR2) comprising the sequence of YINPYNGRTGYNQKFKA (SEQ ID NO: 3342), and a heavy chain complementarity determining region 3 (VHCDR3) comprising the sequence of WDGSSYFDY (SEQ ID NO: 3343), and
(b) a light chain variable region (VL) comprising a light chain complementarity determining region 1 (VLCDR1) comprising the sequence of SASSSVSYMH (SEQ ID NO: 3344), a light chain complementarity determining region 2 (VLCDR2) comprising the sequence of EISKLAS (SEQ ID NO: 3345), a light chain complementarity determining region 3 (VLCDR3) comprising the sequence of QQWNYPLLT (SEQ ID NO: 3346).

276. The composition of claim 275, wherein the VH of the anti-TRBV5 comprises the sequence of SEQ ID NO: 3445.

277. The composition of claim 275, wherein the VL of the anti-TRBV5 comprises the sequence of SEQ ID NO: 3446.

278. The composition of claim 264, wherein the agent further comprises an anti-T cell receptor alpha chain variable region (TCRαV) antibody molecule.

279. The composition of claim 278, wherein the anti-TCRαV antibody molecule is an anti-TRAV21.

280. A method of treating a disease or condition in a subject in need thereof comprising administering to the subject a therapeutically effective amount of the composition of claim 264.

281. A composition comprising an agent that comprises an antigen binding domain that binds to a T cell receptor variable beta region 5 (TCRβV5), wherein the antigen binding domain comprises:
(a) a heavy chain variable region (VH) comprising a heavy chain complementarity determining region 1 (VHCDR1) comprising the sequence of GYTFTDYNIH (SEQ ID NO: 3341), a heavy chain complementarity determining region 2 (VHCDR2) comprising the sequence of YINPYNGRTGYNQKFKA (SEQ ID NO: 3342), and a heavy chain complementarity determining region 3 (VHCDR3) comprising the sequence of WDGSSYFDY (SEQ ID NO: 3343), and
(b) a light chain variable region (VL) comprising a light chain complementarity determining region 1 (VLCDR1) comprising the sequence of SASSSVSYMH (SEQ ID NO: 3344), a light chain complementarity determining region 2 (VLCDR2) comprising the sequence of EISKLAS (SEQ ID NO: 3345), a light chain complementarity determining region 3 (VLCDR3) comprising the sequence of QQWNYPLLT (SEQ ID NO: 3346),
wherein the VH comprises a sequence having at least 96% sequence identity to SEQ ID NO: 3445, or
wherein the VL comprises a sequence having at least 95% sequence identity to SEQ ID NO: 3446.

282. The composition of claim 281, wherein the VH comprises a sequence having at least 98% sequence identity to SEQ ID NO: 3445, and wherein the VL comprises a sequence having at least 98% sequence identity to SEQ ID NO: 3446.

283. The composition of claim 282, wherein the VH comprises SEQ ID NO: 3445, and wherein the VL comprises SEQ ID NO: 3446.

284. The composition of claim 281, wherein the agent further comprises at least one cytokine molecule or a functional fragment or variant thereof.

285. The composition of claim 284, wherein the at least one cytokine molecule or a functional fragment or variant thereof comprises IL-2 or a functional fragment or variant thereof, IL-7 or a functional fragment or variant thereof, IL-12 or a functional fragment or variant thereof, IL-15 or a functional fragment or variant thereof, IL-18 or a functional fragment or variant thereof, IL-21 or a functional fragment or variant thereof, interferon gamma or a functional fragment or variant thereof, or any combination thereof.

286. The composition of claim 285, wherein the at least one cytokine molecule or a functional fragment or variant thereof comprises IL-2 or a functional fragment or variant thereof.

287. The composition of claim 281, wherein the agent further comprises an antigen binding domain that binds to NKp30.

288. The composition of claim 287, wherein the antigen binding domain that binds to NKp30 comprises:
(a) a heavy chain variable region (VH) comprising a heavy chain complementarity determining region 1 (VHCDR1) comprising the sequence of GFSIIRVGYHWN (SEQ ID NO: 502), a heavy chain complementarity determining region 2 (VHCDR2) comprising the sequence of YIHRSGSTSYNPSLKS (SEQ ID NO: 503), and a heavy chain complementarity determining region 3 (VHCDR3) comprising the sequence of GDWHYFDY (SEQ ID NO: 504), and
(b) a light chain variable region (VL) comprising a light chain complementarity determining region 1 (VLCDR1) comprising the sequence of SGEKLSDKYVH (SEQ ID NO: 26), a light chain complementarity determining region 2 (VLCDR2) comprising the sequence of ENDRRPS (SEQ ID NO: 28), a light chain complementarity determining region 3 (VLCDR3) comprising the sequence of QFWDSTNSAV (SEQ ID NO: 86).

289. A method of treating a disease or condition in a subject in need thereof comprising administering to the subject a therapeutically effective amount of the composition of claim 281.
